(19) United States
(12) Patent Application Publication
(10) Pub. No.: US 2024/0025922 A1
(43) Pub. Date: Jan. 25, 2024

(54) TETRAHYDROPYRIDO[3,4-d]PYRIMIDINES AS HPK1 INHIBITORS

(71) Applicant: Merck Patent GmbH, Darmstadt (DE)

(72) Inventors: Momar TOURE, Billerica, MA (US); Bin LI, Southborough, MA (US); Constantin NEAGU, Belmont, MA (US); Yanping WANG, Billerica, MA (US); Theresa JOHNSON, Salem, MA (US); Andrea UNZUE LOPEZ, Darmstadt (DE); Yufang XIAO, Lexington, MA (US); Emily FRIIS, Somerville, MA (US); Maria DIPOTO, North Billerica, MA (US); Satenig GULER, Watertown, MA (US)

(21) Appl. No.: 18/313,652

(22) Filed: May 8, 2023

Related U.S. Application Data
(60) Provisional application No. 63/383,190, filed on Nov. 10, 2022.
(60) Provisional application No. 63/340,191, filed on May 10, 2022.

(51) Int. Cl. C07D 519/00

(52) U.S. Cl. CPC C07D 519/00

(57) ABSTRACT

Tetrahydropyrido[3,4-d]pyrimidines compounds are provided that are potent HPK1 inhibitors. The compounds are useful to treat or prevent cancer and/or inflammatory and/or autoimmune diseases or symptoms thereof in mammals, particularly humans. The compounds have a chemical structure of the general Formula (I), (II), (III), (IV) or (V) or enantiomers or diastereomers or mixtures thereof or pharmaceutically acceptable salts thereof.

### BACKGROUND OF THE INVENTION

### Cross-Reference to Related Applications

The application claims the benefit of priority of US Provisional Application Nos. 63/383,190, filed Nov. 10, 2022 and 63/340,191, filed May 10, 2022, the entire contents of which are incorporated herein by reference.

### FIELD OF THE INVENTION

### Description of Related Art

Hematopoietic progenitor kinase 1 (HPK1) is a serine/threonine kinase expressed in T cells, B-cells, and dendritic cells (Jr-Wen Shui, Nature Immunology, vol. 8, 2007, pp. 84-91). In T cells, HPK1 acts as a rheostat of T cell activation by regulating the molecular circuits of the T cell receptor (TCR) signaling pathway. HPK1 is recruited to the TCR complex and phosphorylates SLP76 protein leading to its degradation and down-modulation of TCR signal strength. Genetic ablation of HPK1 results in T cell activation, lower TCR threshold, increased proliferation, and elevated levels of pro-inflammatory cytokines such as IL-2, TNF-α, and IFN-γ. Loss of HPK1 expression enhances dendritic cell activation and antigen presentation. (Hemandez S. et al., Cell Reports, vol. 25, 2018, pp. 80-94). HPK1 kinase activity is believed to be critical in conferring suppressive functions of HPK1 in a wide range of immune cells, such as CD8+, CD4+, DC, and NK to regulatory T cells (Tregs). Inactivation of the kinase domain of HPK1 was sufficient to elicit robust anti-tumor immune responses (Liu et al., PLoS One, Mar. 26, 2019). HPK1 knockout (KO) and kinase dead (KD) mice show enhanced T cell function and antitumor efficacy (You D. et. al., J. Immunother. Cancer, 2021). Therefore, pharmacological inhibition of HPK1 has the potential to enhance effector T cell function and antitumor activity.

### SUMMARY OF THE INVENTION

In an embodiment, a compound of Formula I

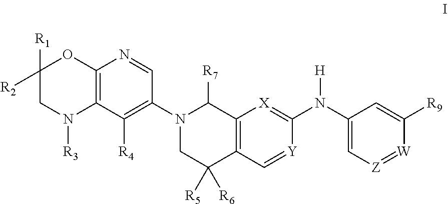

wherein:
R₁ and R₂ are each independently selected from the group consisting of hydrogen and C₁-C₆ alkyl, C₃-C₆ cycloalkyl and C₁-C₆ haloalkyl;
R₁ and R₂ together with the carbon to which they are attached can be taken together to form a C₃-C₆ carbocyclic ring;
R₃ is selected from the group consisting of hydrogen and C₁-C₆ alkyl;
R₄ is selected from the group consisting of hydrogen, halogen, C₁-C₆ alkyl, C₃-C₆ cycloalkyl and C₁-C₆ haloalkyl;
R₅ and R₆ are each independently selected from the group consisting of hydrogen, halogen, C₁-C₆ alkyl, C₃-C₆ cycloalkyl and C₁-C₆ haloalkyl;
R₇ is selected from the group consisting of hydrogen and C₁-C₆ alkyl;
X is selected from the group consisting of N and CH;
Y is selected from the group consisting of N and CH;
W is selected from the group consisting of N and CR₈;
Z is selected from the group consisting of N and CH;
R₈ and R₉ are independently selected from the group consisting of hydrogen, halogen, optionally substituted C₁-C₆ alkyl, optionally substituted C₃-C₆ cycloalkyl, C₁-C₆ haloalkyl, optionally substituted C₂-C₈ heterocyclic, optionally substituted heteroaromatic, optionally substituted C₆-C₁₄ aromatic, —(CH₂)ₘ—NR′R″, —NR′—(CH₂)ₘ—NR′R″, —(CH₂)ₙ—OR₁₁, —O—(CH₂)ₙ—OR₁₁;

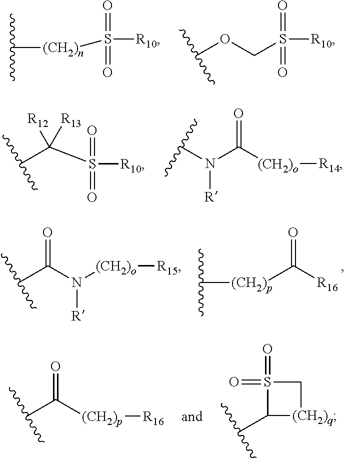

R₁₀ is selected independently for each occurrence from the group consisting C₁-C₆ alkyl, —NR′R″, —(CH₂)ₚ—OR₁₁, —(CH₂)ₙ—O—(CH₂)ₙ—OR₁₁ and —(CH₂)ₘ—NR′R″;
R′ and R″ are selected independently for each occurrence from the group consisting of hydrogen, C₁-C₆ alkyl and C₃-C₆ cycloalkyl;
R₁₂ and R₁₃ are independently selected for each occurrence from the group consisting of hydrogen and C₁-C₆ alkyl; R₁₂ and R₁₃ together with the carbon to which they are attached can be taken together to form a C₃-C₆ carbocyclic ring;
R₁₁, R₁₄, R₁₅ and R₁₆ are independently selected for each occurrence from the group consisting of hydrogen, C₁-C₆ alkyl, optionally substituted C₂-C₈ heterocyclic, optionally substituted heteroaromatic, optionally substituted C₆-C₁₄ aromatic;
m, n, o, p and q are each independently selected from the group consisting of 0, 1, 2, 3, 4, 5 and 6;
or enantiomers or diastereomers or mixtures thereof or pharmaceutically acceptable salts thereof is provided.

In an embodiment a compound of Formula I wherein R₁ and R₂ are hydrogen is provided.

In an embodiment a compound of Formula I wherein R₃ is selected from the group consisting of hydrogen and —CH₃ is provided.

In an embodiment a compound of Formula I wherein R₄ is selected from the group consisting of hydrogen,

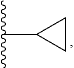 [SMILES: *C1CC1]

Cl, —CH₂CH₃ and —CH₃ is provided.

In an embodiment a compound of Formula I wherein R₅ and R₆ are hydrogen is provided.

In an embodiment a compound of Formula I wherein R₇ is selected from the group consisting of hydrogen and —CH₃ is provided.

In an embodiment a compound of Formula I wherein X and Y are CH is provided.

In an embodiment a compound of Formula I wherein X and Y are N is provided.

In an embodiment a compound of Formula I wherein X is N and Y is CH is provided.

In an embodiment a compound of Formula I wherein W is CR₈ is provided.

In an embodiment a compound of Formula I wherein R₈ is selected from the group consisting of

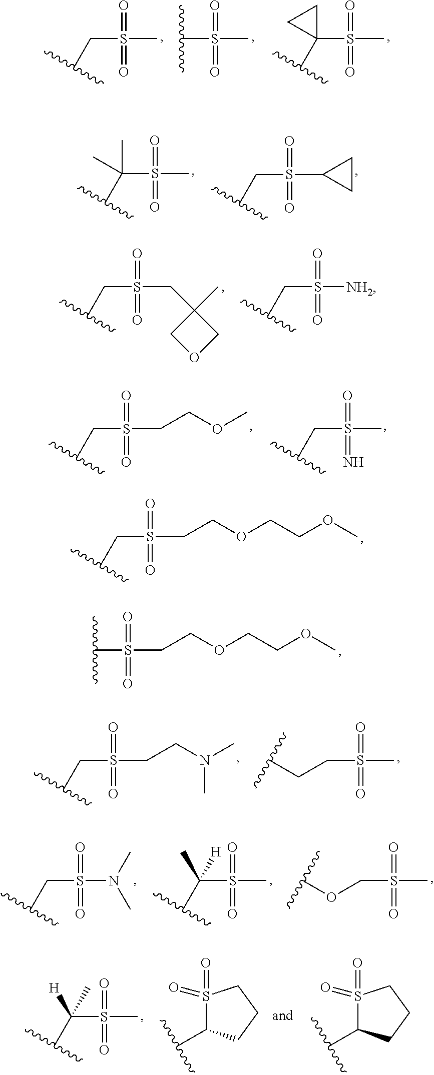

is provided.

In an embodiment a compound of Formula I wherein R₈ is selected from the group consisting of

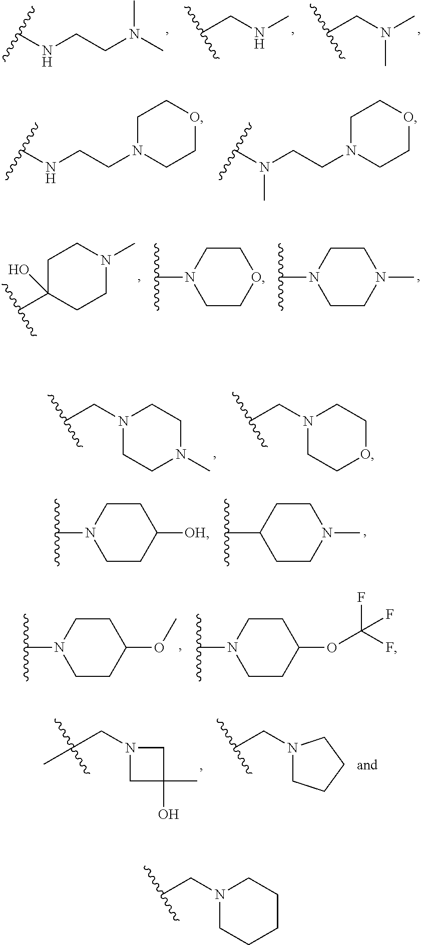

is provided.

In an embodiment a compound of Formula I wherein R₈ is selected from the group consisting of

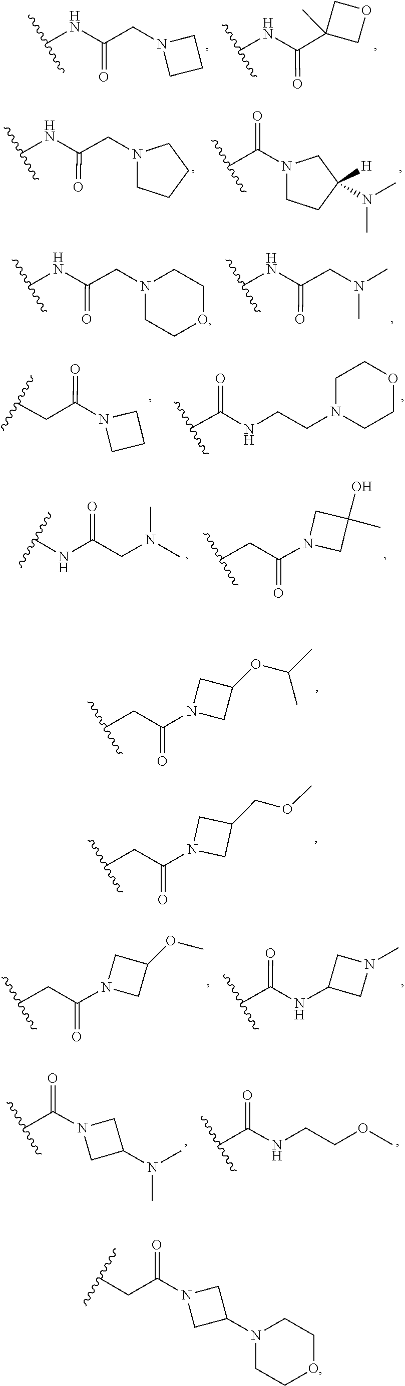

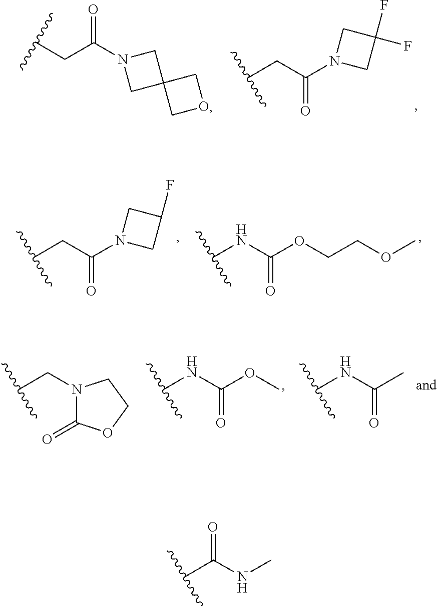

is provided.

In an embodiment a compound of Formula I wherein R₈ is selected from the group consisting of

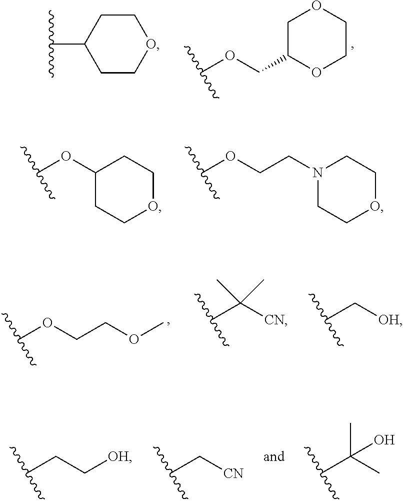

is provided.

In an embodiment a compound of Formula I wherein R₈ is selected from the group consisting of

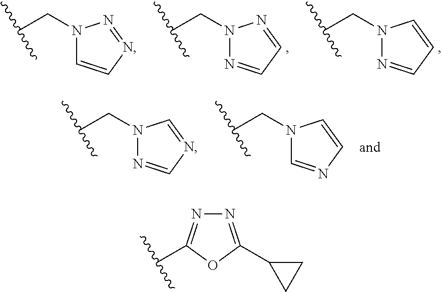

is provided.

In an embodiment a compound of Formula I wherein R₉ is selected from the group consisting of hydrogen, F, Cl, Br, I, —CF₃, —CH₃, —CH₂CH₃,

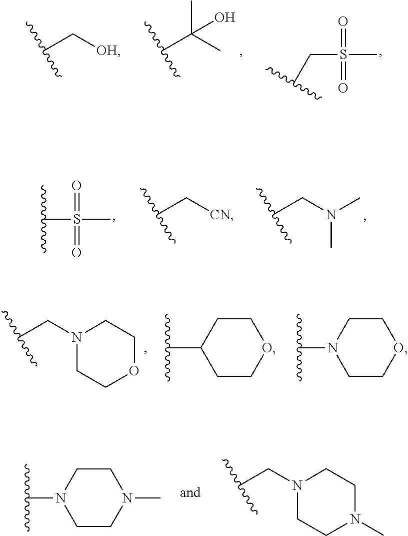

is provided.

In an embodiment a compound of the Formula II

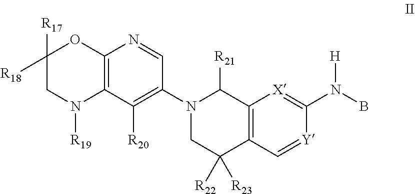

wherein:
B is selected from the group consisting of

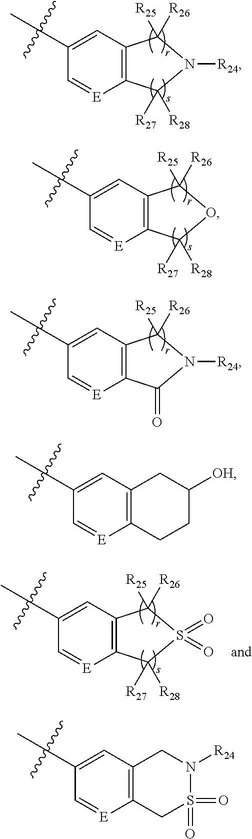

R₁₇ and R₁₈ are each independently selected from the group consisting of hydrogen and C₁-C₆ alkyl, C₃-C₆ cycloalkyl and C₁-C₆ haloalkyl;
R₁₇ and R₁₈ together with the carbon to which they are attached can be taken together to form a C₃-C₆ carbocyclic ring;
R₁₉ is selected from the group consisting of hydrogen and C₁-C₆ alkyl;
R₂₀ is selected from the group consisting of hydrogen, halogen, C₁-C₆ alkyl, C₃-C₆ cycloalkyl and C₁-C₆ haloalkyl;
R₂₁ is selected from the group consisting of hydrogen and C₁-C₆ alkyl;
R₂₂ and R₂₃ are each independently selected from the group consisting of hydrogen, halogen, C₁-C₆ alkyl, C₃-C₆ cycloalkyl and C₁-C₆ haloalkyl;
R₂₄ is selected from the group consisting of hydrogen, optionally substituted C₁-C₆ alkyl, optionally substituted C₃-C₆ cycloalkyl and C₁-C₆ haloalkyl;
R₂₅, R₂₆, R₂₇ and are R₂₈ are independently selected for each occurrence from the group consisting of hydrogen and C₁-C₆ alkyl;
X′ is selected from the group consisting of N and CH;
Y′ is selected from the group consisting of N and CH;
E is selected from the group consisting of N and CH;
s and r are independently selected from the group consisting of 1, 2 and 3
or enantiomers or diastereomers or mixtures thereof or pharmaceutically acceptable salts thereof is provided.

In an embodiment a compound of the Formula II wherein R₁₇ and R₁₈ are hydrogen is provided.

In an embodiment a compound of the Formula II wherein R₁₉ is selected from the group consisting of hydrogen and —CH₃ is provided.

In an embodiment a compound of the Formula II wherein R₂₀ is selected from the group consisting of hydrogen,

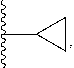 [SMILES: *C1CC1]

Cl, —CH₂CH₃ and —CH₃ is provided.

In an embodiment a compound of the Formula II wherein R₂₂ and R₂₃ are hydrogen is provided.

In an embodiment a compound of the Formula II wherein R₂₁ is selected from the group consisting of hydrogen and —CH₃ is provided.

In an embodiment a compound of the Formula II wherein X′ and Y′ are CH is provided.

In an embodiment a compound of the Formula II wherein X′ and Y′ are N is provided.

In an embodiment a compound of the Formula II wherein X′ is N and Y′ is CH is provided.

In an embodiment a compound of the Formula II wherein r and s are 1 is provided.

In an embodiment a compound of the Formula II wherein R₂₅, R₂₆, R₂₇ and are R₂₈ are hydrogen is provided.

In an embodiment a compound of the Formula II wherein R₂₅ and R₂₆ are —CH₃ and R₂₇ and are R₂₈ are hydrogen is provided.

In an embodiment a compound of the Formula II wherein R₂₄ is selected from hydrogen, —CH₃, —CF₃, CH₂F₂ and

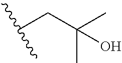 [SMILES: *CC(C)(C)O]

is provided.

In an embodiment a compound of the Formula III

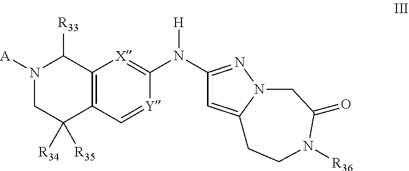

wherein:
A is selected from

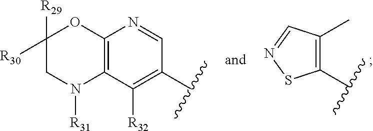

R₂₉ and R₃₀ are each independently selected from the group consisting of hydrogen and C₁-C₆ alkyl, C₃-C₆ cycloalkyl and C₁-C₆ haloalkyl;
R₂₉ and R₃₀ together with the carbon to which they are attached can be taken together to form a C₃-C₆ carbocyclic ring;
R₃₁ is selected from the group consisting of hydrogen and C₁-C₆ alkyl;
R₃₂ is selected from the group consisting of hydrogen, halogen, C₁-C₆ alkyl, C₃-C₆ cycloalkyl and C₁-C₆ haloalkyl;
R₃₃ is selected from the group consisting of hydrogen and C₁-C₆ alkyl;
R₃₄ and R₃₅ are each independently selected from the group consisting of hydrogen, halogen, C₁-C₆ alkyl, C₃-C₆ cycloalkyl and C₁-C₆ haloalkyl;
R₃₆ is selected from the group consisting of hydrogen, optionally substituted C₁-C₆ alkyl, optionally substituted C₃-C₆ cycloalkyl, —(CH₂)ᵤ—OR₃₇ and C₁-C₆ haloalkyl;
R₃₇ is selected from the group consisting of, hydrogen, C₁-C₆ alkyl, C₃-C₆ cycloalkyl and C₁-C₆ haloalkyl;
u is selected from the group consisting of 1, 2, 3, 4, 5 and 6;
X″ is selected from the group consisting of N and CH;
Y″ is selected from the group consisting of N and CH;
or enantiomers or diastereomers or mixtures thereof or pharmaceutically acceptable salts thereof is provided.

In an embodiment a compound of the Formula III wherein R₂₉ and R₃₀ are hydrogen is provided.

In an embodiment a compound of the Formula III wherein R₃₁ is selected from the group consisting of hydrogen and —CH₃ is provided.

In an embodiment a compound of the Formula III wherein R₃₂ is selected from the group consisting of hydrogen,

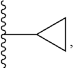 [SMILES: *C1CC1]

Cl, —CH₂CH₃ and —CH₃ is provided.

In an embodiment a compound of the Formula III wherein R₃₄ and R₃₅ are hydrogen is provided.

In an embodiment a compound of the Formula III wherein R₃₃ is selected from the group consisting of hydrogen and —CH₃ is provided.

In an embodiment a compound of the Formula III wherein X″ and Y″ are CH is provided.

In an embodiment a compound of the Formula III wherein X″ and Y″ are N is provided.

In an embodiment a compound of the Formula III wherein X″ is N and Y″ is CH is provided.

In an embodiment a compound of the Formula III wherein R₃₆ is selected from hydrogen, —CH₃, —CH₂CH₂OCH₃, —CH₂CH₂F₂,

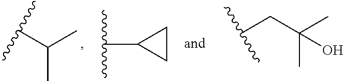

is provided.

In an embodiment a compound of the Formula IV

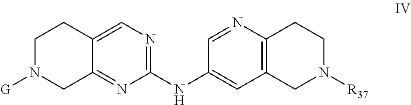

wherein:
G is selected from the group consisting of

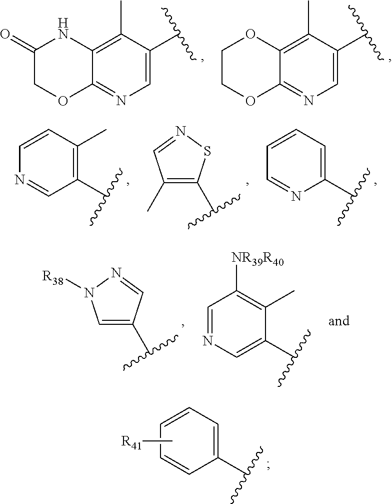

R₃₇, R₃₈, R₃₉ and R₄₀ are independently selected for each occurrence from the group consisting of hydrogen, C₁-C₆ alkyl, and C₃-C₆ cycloalkyl;

R₄₁ is independently selected for each position capable of substitution from the group consisting of hydrogen, halogen, —O—C₁-C₆ alkyl, C₁-C₆ haloalkyl, C₁-C₆ alkyl and C₃-C₆ cycloalkyl; or enantiomers or diastereomers or mixtures thereof or pharmaceutically acceptable salts thereof is provided.

In an embodiment a compound of the Formula IV wherein R₃₇ is —CH₃ is provided.

In an embodiment a compound of the Formula IV wherein R₃₈ is —CH₃ is provided.

In an embodiment a compound of the Formula IV wherein R₃₉ and R₄₀ is hydrogen is provided.

In an embodiment a compound of the Formula IV wherein R₄₁ is selected from the group consisting of hydrogen, —OCH₃ and F is provided.

In an embodiment a compound of the Formula V

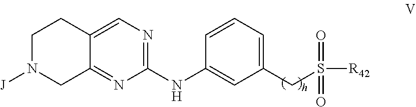

wherein:
J is selected from the group consisting of

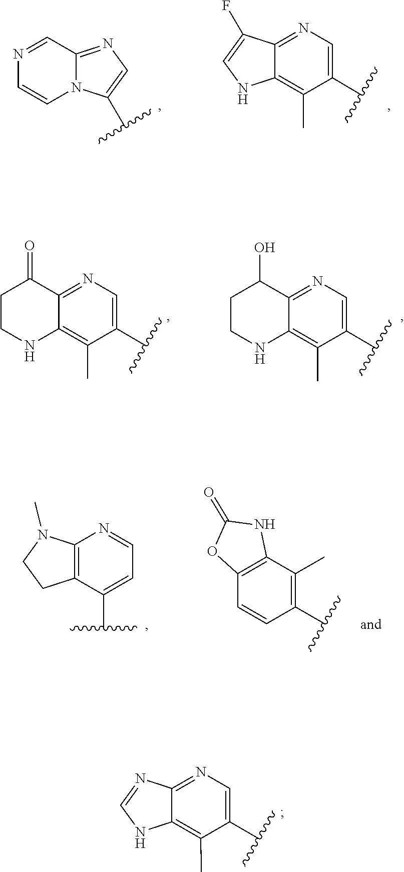

h is 1, 2 or 3
R₄₂ is selected from the group consisting of C₁-C₆ alkyl and C₃-C₆ cycloalkyl; or enantiomers or diastereomers or mixtures thereof or pharmaceutically acceptable salts thereof is provided.

In an embodiment a compound of the Formula V wherein h is 1 is provided.

In an embodiment a compound of the Formula V wherein R₄₂ is —CH₃ is provided.

In an embodiment a compound of the Formula I selected from the group consisting of

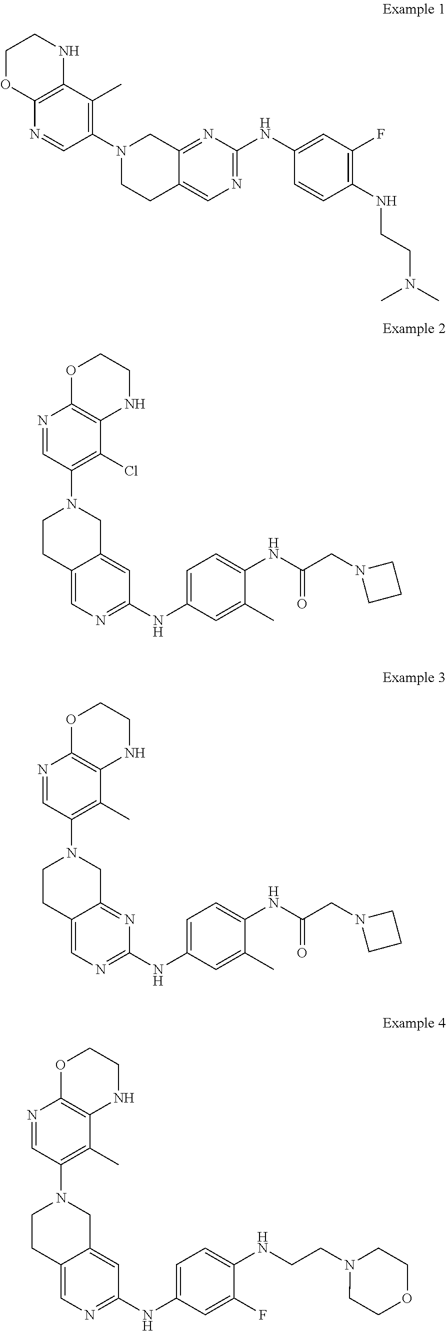 [SMILES: Cc1c(N2CCc3cnc(Nc4ccc(NCCN(C)C)c(F)c4)nc3C2)cnc2c1NCCO2 ; Cc1cc(Nc2cc3c(cn2)CCN(c2cnc4c(c2Cl)NCCO4)C3)ccc1NC(=O)CN1CCC1 ; Cc1cc(Nc2ncc3c(n2)CN(c2cnc4c(c2C)NCCO4)CC3)ccc1NC(=O)CN1CCC1 ; Cc1c(N2CCc3cnc(Nc4ccc(NCCN5CCOCC5)c(F)c4)cc3C2)cnc2c1NCCO2]

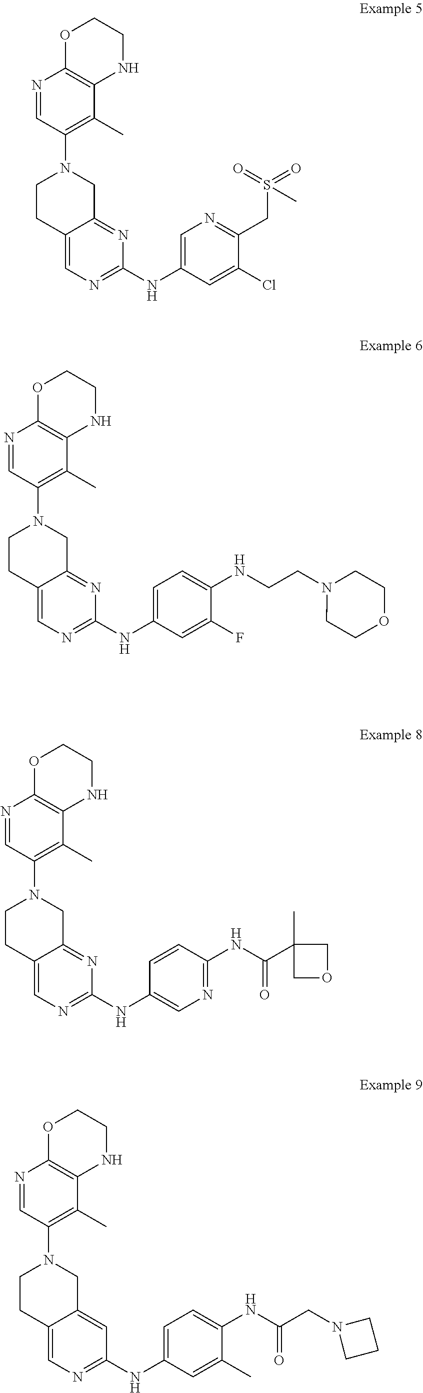 [SMILES: Cc1c(N2CCc3cnc(Nc4cnc(CS(C)(=O)=O)c(Cl)c4)nc3C2)cnc2c1NCCO2 ; Cc1c(N2CCc3cnc(Nc4ccc(NCCN5CCOCC5)c(F)c4)nc3C2)cnc2c1NCCO2 ; Cc1c(N2CCc3cnc(Nc4ccc(NC(=O)C5(C)COC5)nc4)nc3C2)cnc2c1NCCO2 ; Cc1cc(Nc2cc3c(cn2)CCN(c2cnc4c(c2C)NCCO4)C3)ccc1NC(=O)CN1CCC1]

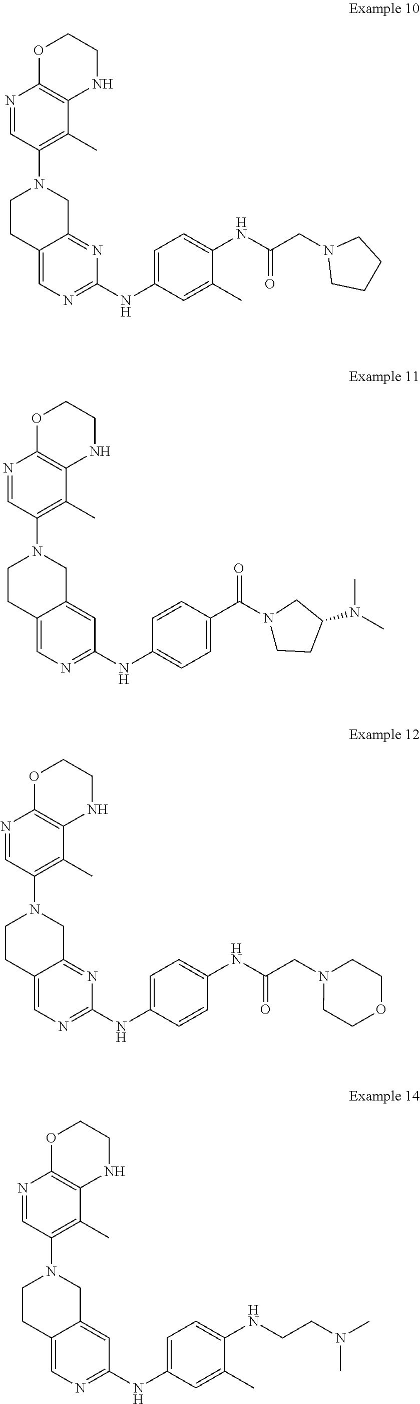 [SMILES: Cc1cc(Nc2ncc3c(n2)CN(c2cnc4c(c2C)NCCO4)CC3)ccc1NC(=O)CN1CCCC1 ; Cc1c(N2CCc3cnc(Nc4ccc(C(=O)N5CC[C@@H](N(C)C)C5)cc4)cc3C2)cnc2c1NCCO2 ; Cc1c(N2CCc3cnc(Nc4ccc(NC(=O)CN5CCOCC5)cc4)nc3C2)cnc2c1NCCO2 ; Cc1cc(Nc2cc3c(cn2)CCN(c2cnc4c(c2C)NCCO4)C3)ccc1NCCN(C)C]

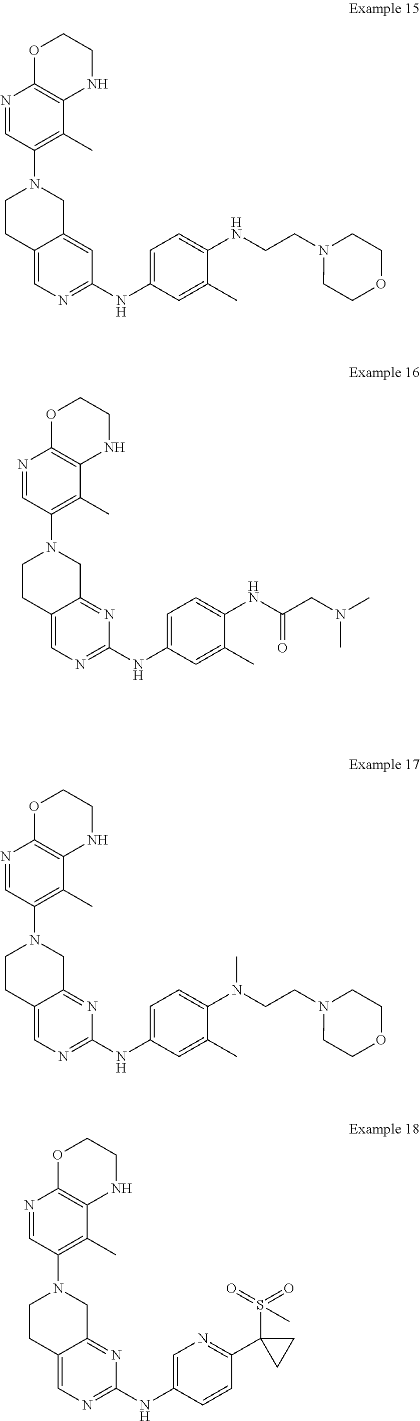 [SMILES: Cc1cc(Nc2cc3c(cn2)CCN(c2cnc4c(c2C)NCCO4)C3)ccc1NCCN1CCOCC1 ; Cc1cc(Nc2ncc3c(n2)CN(c2cnc4c(c2C)NCCO4)CC3)ccc1NC(=O)CN(C)C ; Cc1cc(Nc2ncc3c(n2)CN(c2cnc4c(c2C)NCCO4)CC3)ccc1N(C)CCN1CCOCC1 ; Cc1c(N2CCc3cnc(Nc4ccc(C5(S(C)(=O)=O)CC5)nc4)nc3C2)cnc2c1NCCO2]

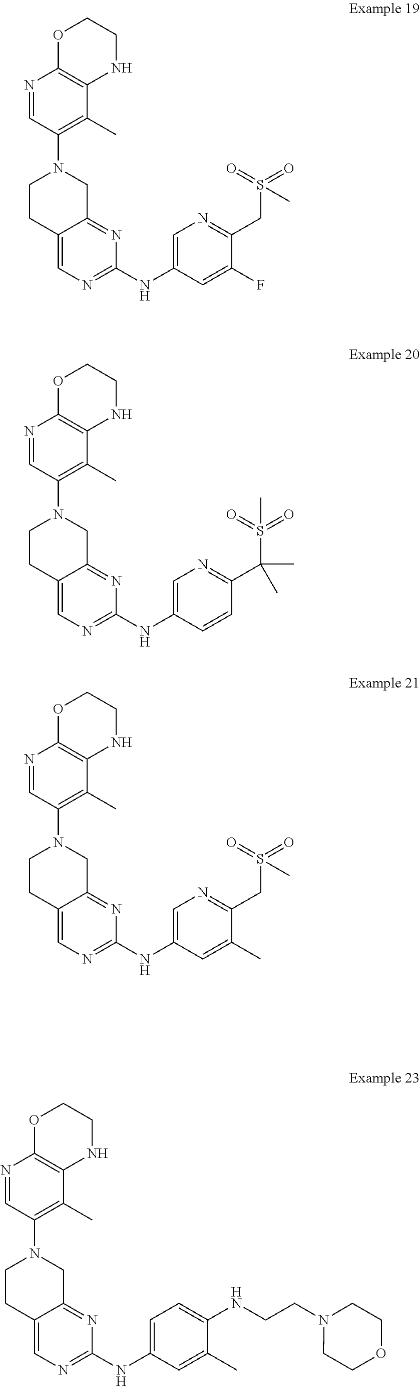 [SMILES: Cc1c(N2CCc3cnc(Nc4cnc(CS(C)(=O)=O)c(F)c4)nc3C2)cnc2c1NCCO2 ; Cc1c(N2CCc3cnc(Nc4ccc(C(C)(C)S(C)(=O)=O)nc4)nc3C2)cnc2c1NCCO2 ; Cc1cc(Nc2ncc3c(n2)CN(c2cnc4c(c2C)NCCO4)CC3)cnc1CS(C)(=O)=O ; Cc1cc(Nc2ncc3c(n2)CN(c2cnc4c(c2C)NCCO4)CC3)ccc1NCCN1CCOCC1]

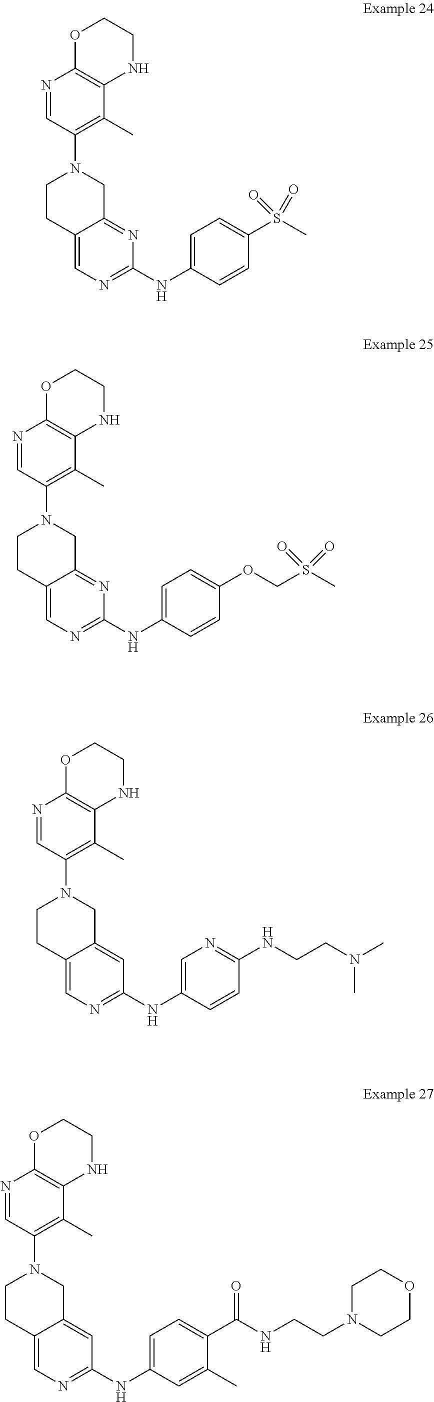 [SMILES: Cc1c(N2CCc3cnc(Nc4ccc(S(C)(=O)=O)cc4)nc3C2)cnc2c1NCCO2 ; Cc1c(N2CCc3cnc(Nc4ccc(OCS(C)(=O)=O)cc4)nc3C2)cnc2c1NCCO2 ; Cc1c(N2CCc3cnc(Nc4ccc(NCCN(C)C)nc4)cc3C2)cnc2c1NCCO2 ; Cc1cc(Nc2cc3c(cn2)CCN(c2cnc4c(c2C)NCCO4)C3)ccc1C(=O)NCCN1CCOCC1]

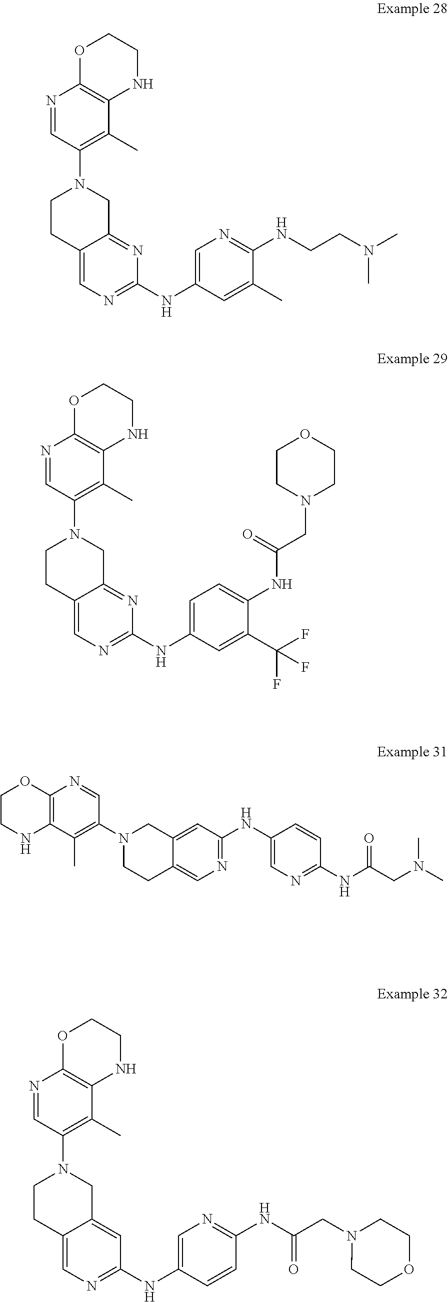 [SMILES: Cc1cc(Nc2ncc3c(n2)CN(c2cnc4c(c2C)NCCO4)CC3)cnc1NCCN(C)C ; Cc1c(N2CCc3cnc(Nc4ccc(NC(=O)CN5CCOCC5)c(C(F)(F)F)c4)nc3C2)cnc2c1NCCO2 ; Cc1c(N2CCc3cnc(Nc4ccc(NC(=O)CN(C)C)nc4)cc3C2)cnc2c1NCCO2 ; Cc1c(N2CCc3cnc(Nc4ccc(NC(=O)CN5CCOCC5)nc4)cc3C2)cnc2c1NCCO2]

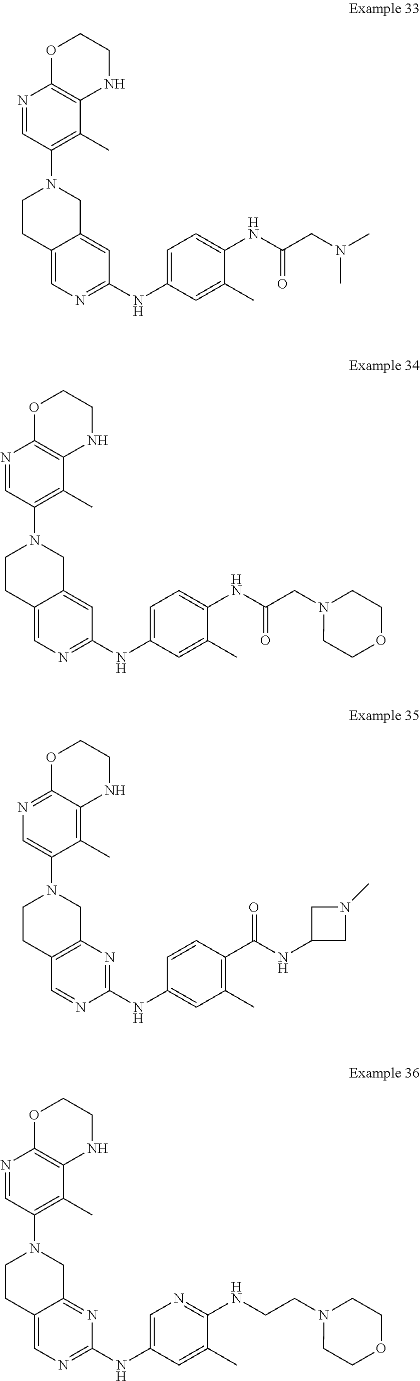 [SMILES: Cc1cc(Nc2cc3c(cn2)CCN(c2cnc4c(c2C)NCCO4)C3)ccc1NC(=O)CN(C)C ; Cc1cc(Nc2cc3c(cn2)CCN(c2cnc4c(c2C)NCCO4)C3)ccc1NC(=O)CN1CCOCC1 ; Cc1cc(Nc2ncc3c(n2)CN(c2cnc4c(c2C)NCCO4)CC3)ccc1C(=O)NC1CN(C)C1 ; Cc1cc(Nc2ncc3c(n2)CN(c2cnc4c(c2C)NCCO4)CC3)cnc1NCCN1CCOCC1]

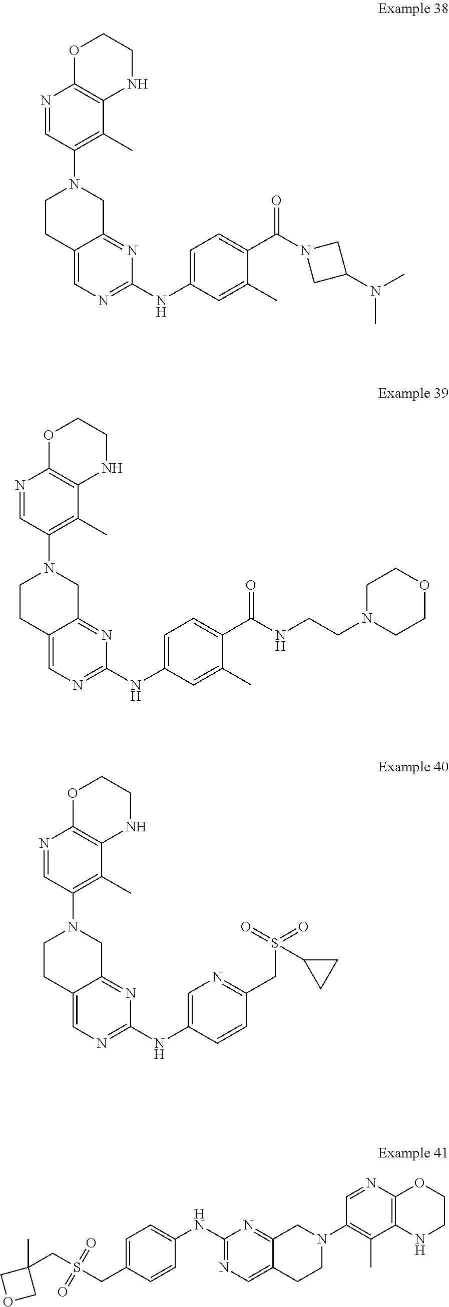 [SMILES: Cc1cc(Nc2ncc3c(n2)CN(c2cnc4c(c2C)NCCO4)CC3)ccc1C(=O)N1CC(N(C)C)C1 ; Cc1cc(Nc2ncc3c(n2)CN(c2cnc4c(c2C)NCCO4)CC3)ccc1C(=O)NCCN1CCOCC1 ; Cc1c(N2CCc3cnc(Nc4ccc(CS(=O)(=O)C5CC5)nc4)nc3C2)cnc2c1NCCO2 ; Cc1c(N2CCc3cnc(Nc4ccc(CS(=O)(=O)CC5(C)COC5)cc4)nc3C2)cnc2c1NCCO2]

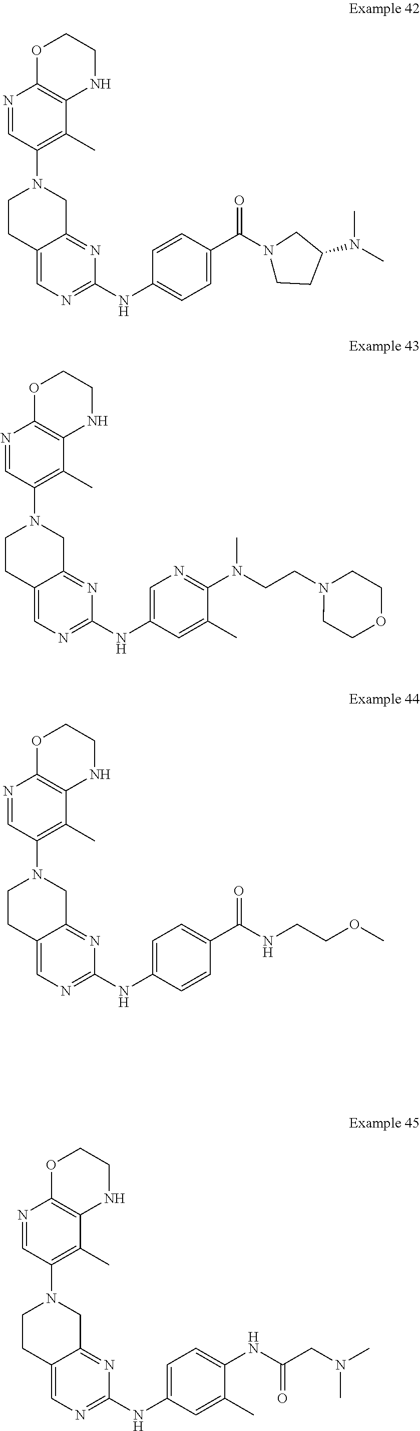 [SMILES: Cc1c(N2CCc3cnc(Nc4ccc(C(=O)N5CC[C@@H](N(C)C)C5)cc4)nc3C2)cnc2c1NCCO2 ; Cc1cc(Nc2ncc3c(n2)CN(c2cnc4c(c2C)NCCO4)CC3)cnc1N(C)CCN1CCOCC1 ; COCCNC(=O)c1ccc(Nc2ncc3c(n2)CN(c2cnc4c(c2C)NCCO4)CC3)cc1 ; Cc1cc(Nc2ncc3c(n2)CN(c2cnc4c(c2C)NCCO4)CC3)ccc1NC(=O)CN(C)C]

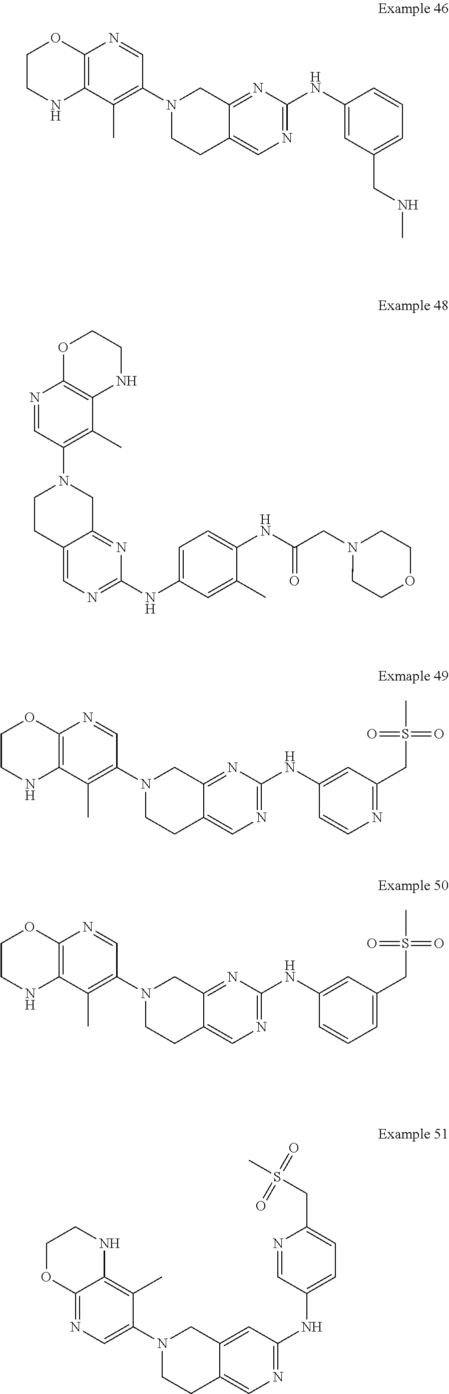 [SMILES: CNCc1cccc(Nc2ncc3c(n2)CN(c2cnc4c(c2C)NCCO4)CC3)c1 ; Cc1cc(Nc2ncc3c(n2)CN(c2cnc4c(c2C)NCCO4)CC3)ccc1NC(=O)CN1CCOCC1 ; Cc1c(N2CCc3cnc(Nc4ccnc(CS(C)(=O)=O)c4)nc3C2)cnc2c1NCCO2 ; Cc1c(N2CCc3cnc(Nc4cccc(CS(C)(=O)=O)c4)nc3C2)cnc2c1NCCO2 ; Cc1c(N2CCc3cnc(Nc4ccc(CS(C)(=O)=O)nc4)cc3C2)cnc2c1NCCO2]

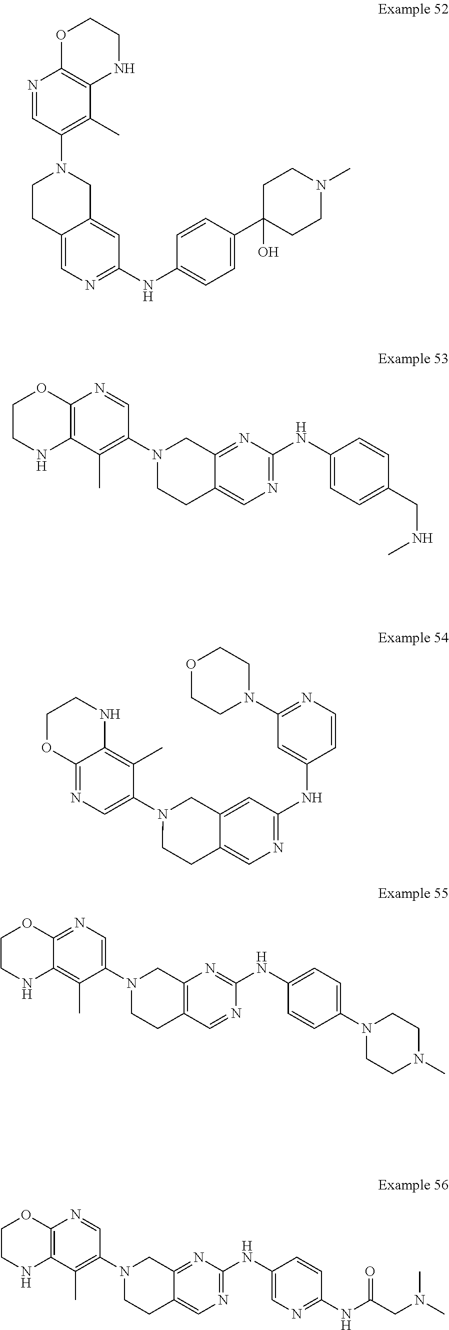 [SMILES: Cc1c(N2CCc3cnc(Nc4ccc(C5(O)CCN(C)CC5)cc4)cc3C2)cnc2c1NCCO2 ; CNCc1ccc(Nc2ncc3c(n2)CN(c2cnc4c(c2C)NCCO4)CC3)cc1 ; Cc1c(N2CCc3cnc(Nc4ccnc(N5CCOCC5)c4)cc3C2)cnc2c1NCCO2 ; Cc1c(N2CCc3cnc(Nc4ccc(N5CCN(C)CC5)cc4)nc3C2)cnc2c1NCCO2 ; Cc1c(N2CCc3cnc(Nc4ccc(NC(=O)CN(C)C)nc4)nc3C2)cnc2c1NCCO2]

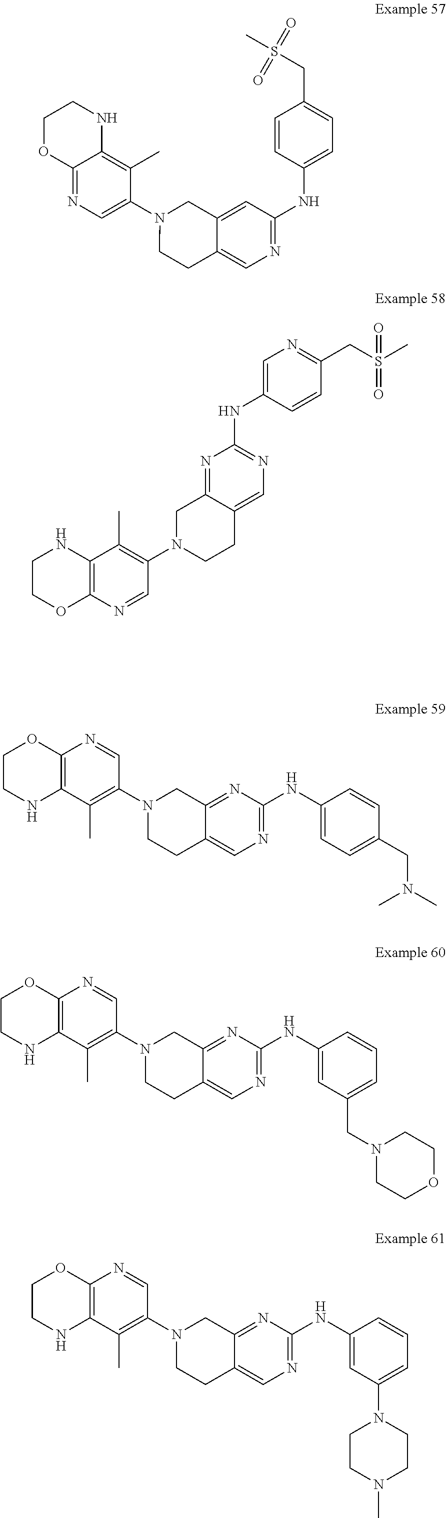 [SMILES: Cc1c(N2CCc3cnc(Nc4ccc(CS(C)(=O)=O)cc4)cc3C2)cnc2c1NCCO2 ; Cc1c(N2CCc3cnc(Nc4ccc(CS(C)(=O)=O)nc4)nc3C2)cnc2c1NCCO2 ; Cc1c(N2CCc3cnc(Nc4ccc(CN(C)C)cc4)nc3C2)cnc2c1NCCO2 ; Cc1c(N2CCc3cnc(Nc4cccc(CN5CCOCC5)c4)nc3C2)cnc2c1NCCO2 ; Cc1c(N2CCc3cnc(Nc4cccc(N5CCN(C)CC5)c4)nc3C2)cnc2c1NCCO2]

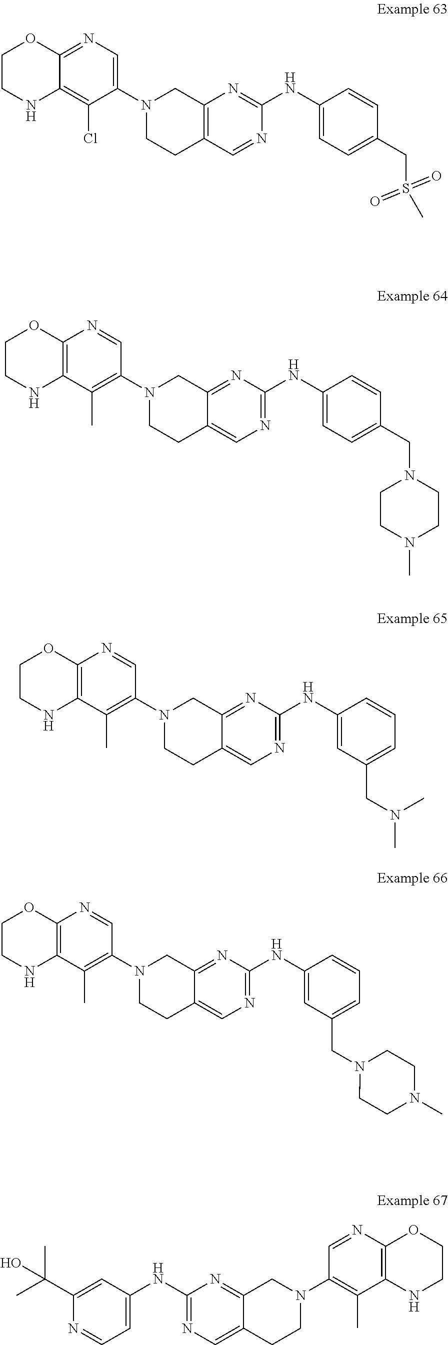

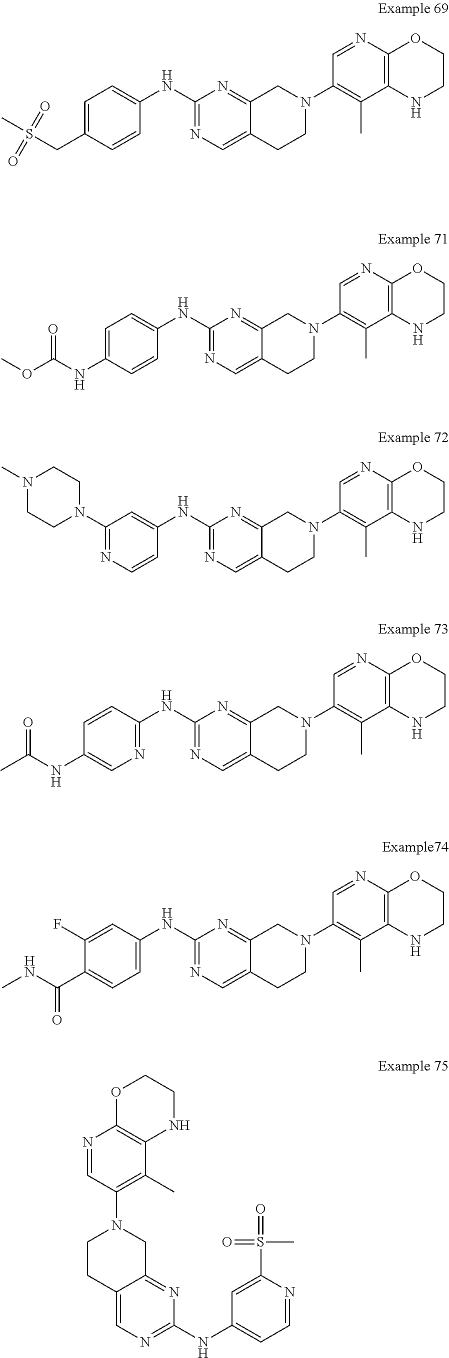 [SMILES: Cc1c(N2CCc3cnc(Nc4ccc(CS(C)(=O)=O)cc4)nc3C2)cnc2c1NCCO2 ; COC(=O)Nc1ccc(Nc2ncc3c(n2)CN(c2cnc4c(c2C)NCCO4)CC3)cc1 ; Cc1c(N2CCc3cnc(Nc4ccnc(N5CCN(C)CC5)c4)nc3C2)cnc2c1NCCO2 ; CC(=O)Nc1ccc(Nc2ncc3c(n2)CN(c2cnc4c(c2C)NCCO4)CC3)nc1 ; CNC(=O)c1ccc(Nc2ncc3c(n2)CN(c2cnc4c(c2C)NCCO4)CC3)cc1F ; Cc1c(N2CCc3cnc(Nc4ccnc(S(C)(=O)=O)c4)nc3C2)cnc2c1NCCO2]

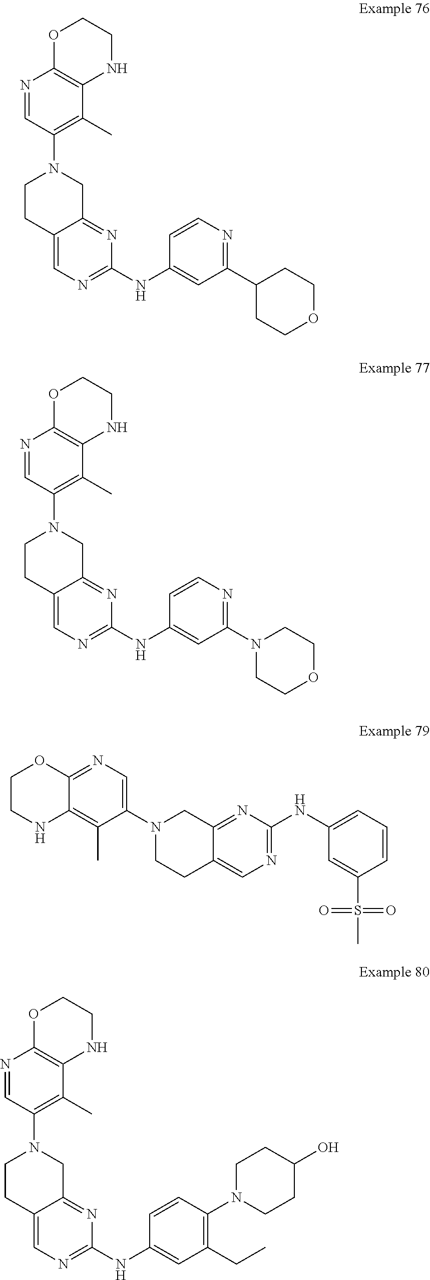 [SMILES: Cc1c(N2CCc3cnc(Nc4ccnc(C5CCOCC5)c4)nc3C2)cnc2c1NCCO2 ; Cc1c(N2CCc3cnc(Nc4ccnc(N5CCOCC5)c4)nc3C2)cnc2c1NCCO2 ; Cc1c(N2CCc3cnc(Nc4cccc(S(C)(=O)=O)c4)nc3C2)cnc2c1NCCO2 ; CCc1cc(Nc2ncc3c(n2)CN(c2cnc4c(c2C)NCCO4)CC3)ccc1N1CCC(O)CC1]

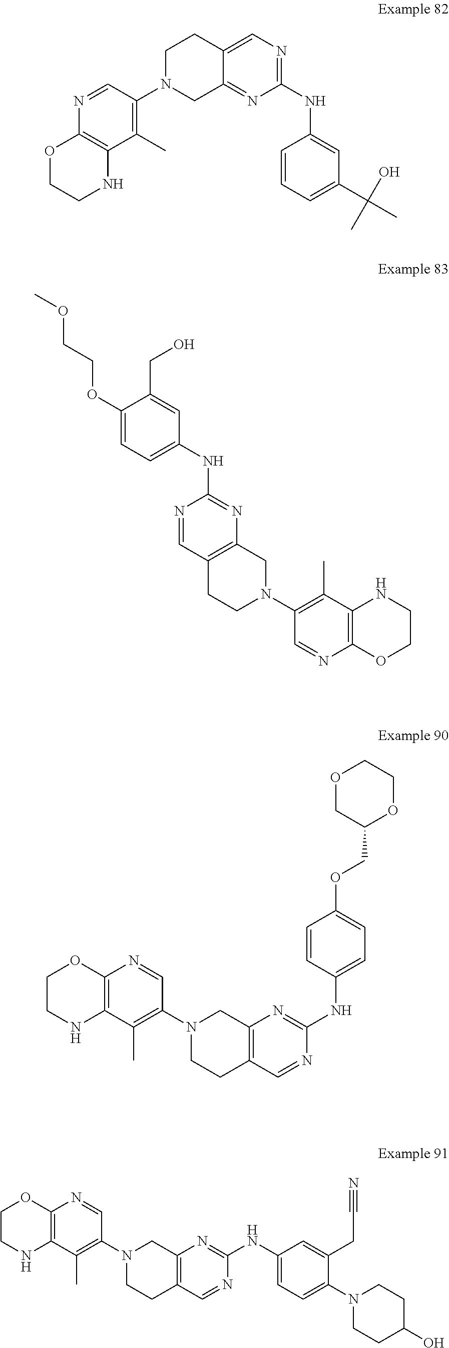

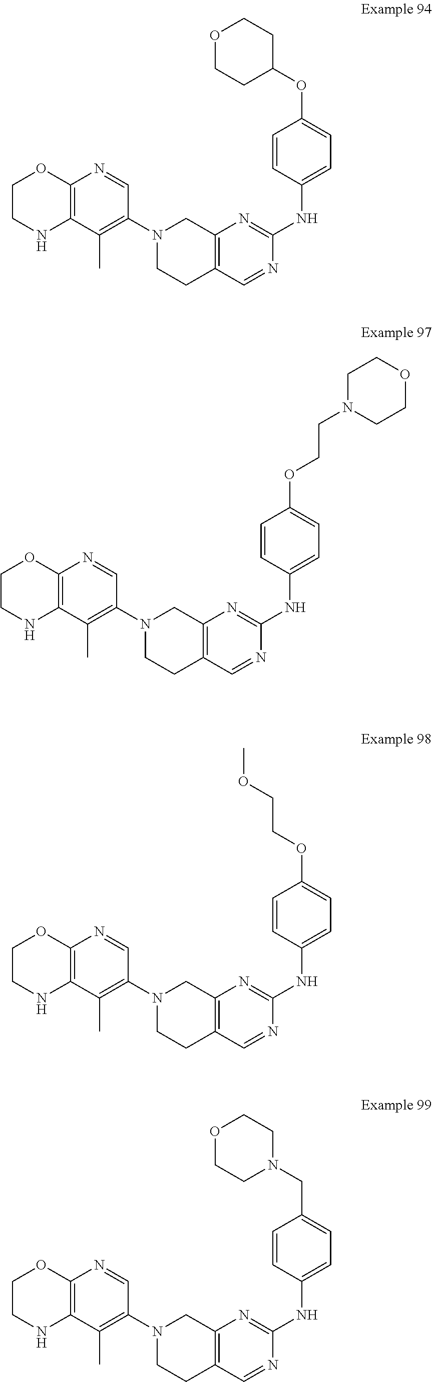 [SMILES: Cc1c(N2CCc3cnc(Nc4ccc(OC5CCOCC5)cc4)nc3C2)cnc2c1NCCO2 ; Cc1c(N2CCc3cnc(Nc4ccc(OCCN5CCOCC5)cc4)nc3C2)cnc2c1NCCO2 ; COCCOc1ccc(Nc2ncc3c(n2)CN(c2cnc4c(c2C)NCCO4)CC3)cc1 ; Cc1c(N2CCc3cnc(Nc4ccc(CN5CCOCC5)cc4)nc3C2)cnc2c1NCCO2]

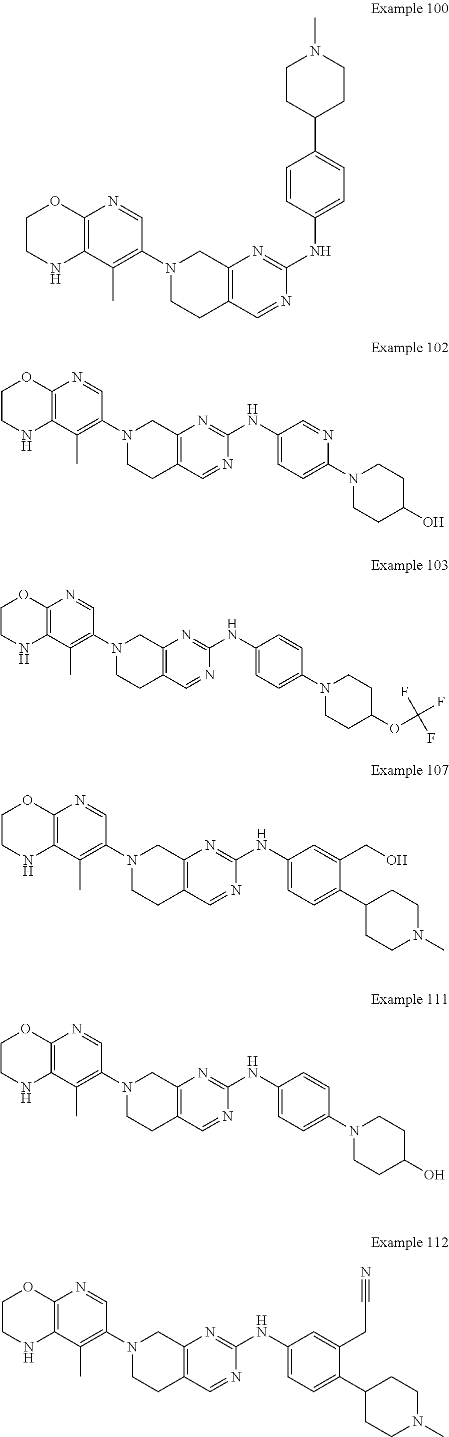 [SMILES: Cc1c(N2CCc3cnc(Nc4ccc(C5CCN(C)CC5)cc4)nc3C2)cnc2c1NCCO2 ; Cc1c(N2CCc3cnc(Nc4ccc(N5CCC(O)CC5)nc4)nc3C2)cnc2c1NCCO2 ; Cc1c(N2CCc3cnc(Nc4ccc(N5CCC(OC(F)(F)F)CC5)cc4)nc3C2)cnc2c1NCCO2 ; Cc1c(N2CCc3cnc(Nc4ccc(C5CCN(C)CC5)c(CO)c4)nc3C2)cnc2c1NCCO2 ; Cc1c(N2CCc3cnc(Nc4ccc(N5CCC(O)CC5)cc4)nc3C2)cnc2c1NCCO2 ; Cc1c(N2CCc3cnc(Nc4ccc(C5CCN(C)CC5)c(CC#N)c4)nc3C2)cnc2c1NCCO2]

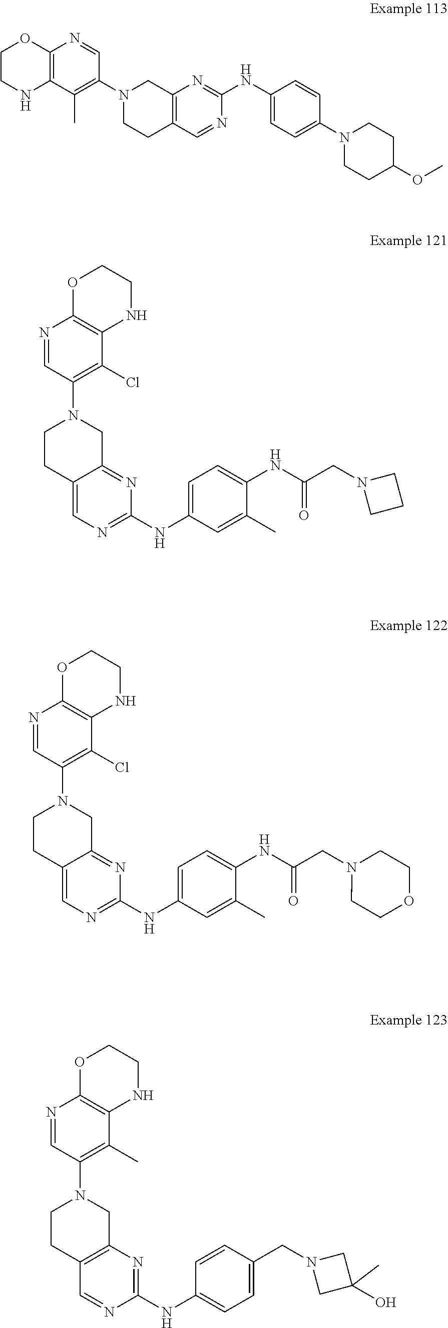 [SMILES: COC1CCN(c2ccc(Nc3ncc4c(n3)CN(c3cnc5c(c3C)NCCO5)CC4)cc2)CC1 ; Cc1cc(Nc2ncc3c(n2)CN(c2cnc4c(c2Cl)NCCO4)CC3)ccc1NC(=O)CN1CCC1 ; Cc1cc(Nc2ncc3c(n2)CN(c2cnc4c(c2Cl)NCCO4)CC3)ccc1NC(=O)CN1CCOCC1 ; Cc1c(N2CCc3cnc(Nc4ccc(CN5CC(C)(O)C5)cc4)nc3C2)cnc2c1NCCO2]

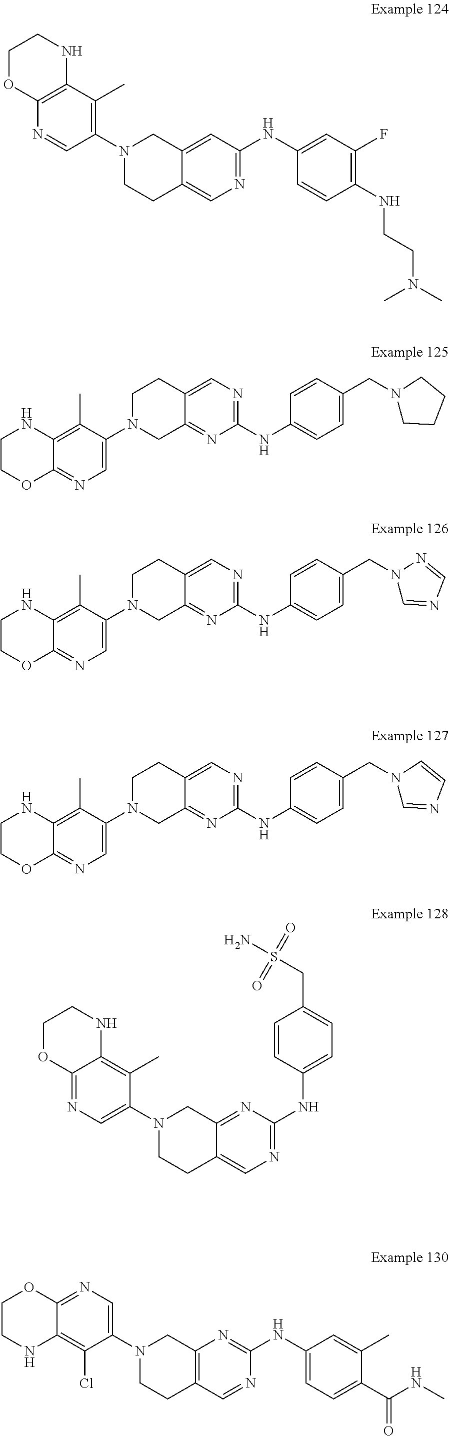 [SMILES: Cc1c(N2CCc3cnc(Nc4ccc(NCCN(C)C)c(F)c4)cc3C2)cnc2c1NCCO2 ; Cc1c(N2CCc3cnc(Nc4ccc(CN5CCCC5)cc4)nc3C2)cnc2c1NCCO2 ; Cc1c(N2CCc3cnc(Nc4ccc(Cn5cncn5)cc4)nc3C2)cnc2c1NCCO2 ; Cc1c(N2CCc3cnc(Nc4ccc(Cn5ccnc5)cc4)nc3C2)cnc2c1NCCO2 ; Cc1c(N2CCc3cnc(Nc4ccc(CS(N)(=O)=O)cc4)nc3C2)cnc2c1NCCO2 ; CNC(=O)c1ccc(Nc2ncc3c(n2)CN(c2cnc4c(c2Cl)NCCO4)CC3)cc1C]

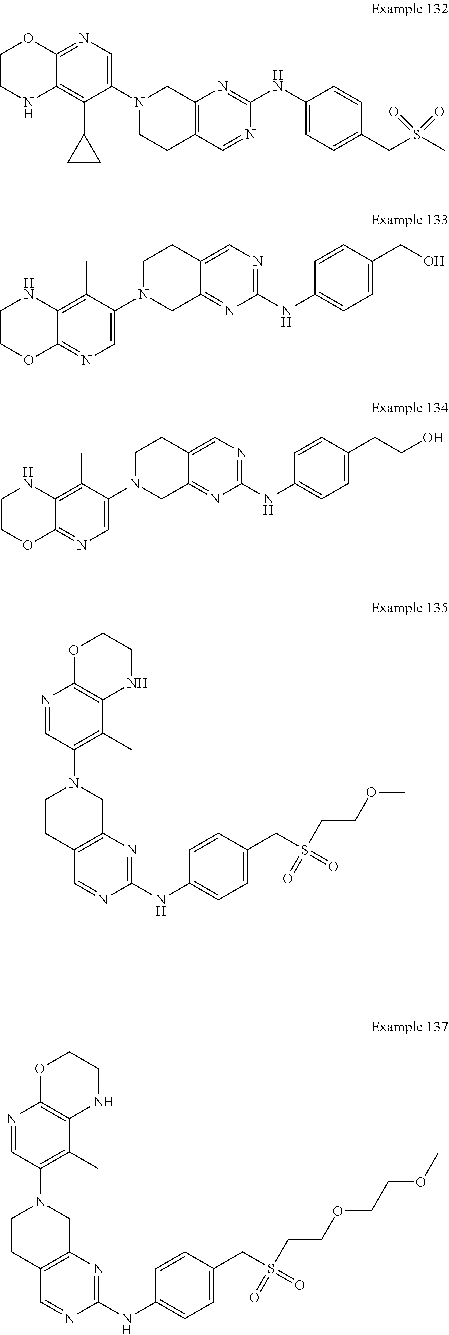 [SMILES: CS(=O)(=O)Cc1ccc(Nc2ncc3c(n2)CN(c2cnc4c(c2C2CC2)NCCO4)CC3)cc1 ; Cc1c(N2CCc3cnc(Nc4ccc(CO)cc4)nc3C2)cnc2c1NCCO2 ; Cc1c(N2CCc3cnc(Nc4ccc(CCO)cc4)nc3C2)cnc2c1NCCO2 ; COCCS(=O)(=O)Cc1ccc(Nc2ncc3c(n2)CN(c2cnc4c(c2C)NCCO4)CC3)cc1 ; COCCOCCS(=O)(=O)Cc1ccc(Nc2ncc3c(n2)CN(c2cnc4c(c2C)NCCO4)CC3)cc1]

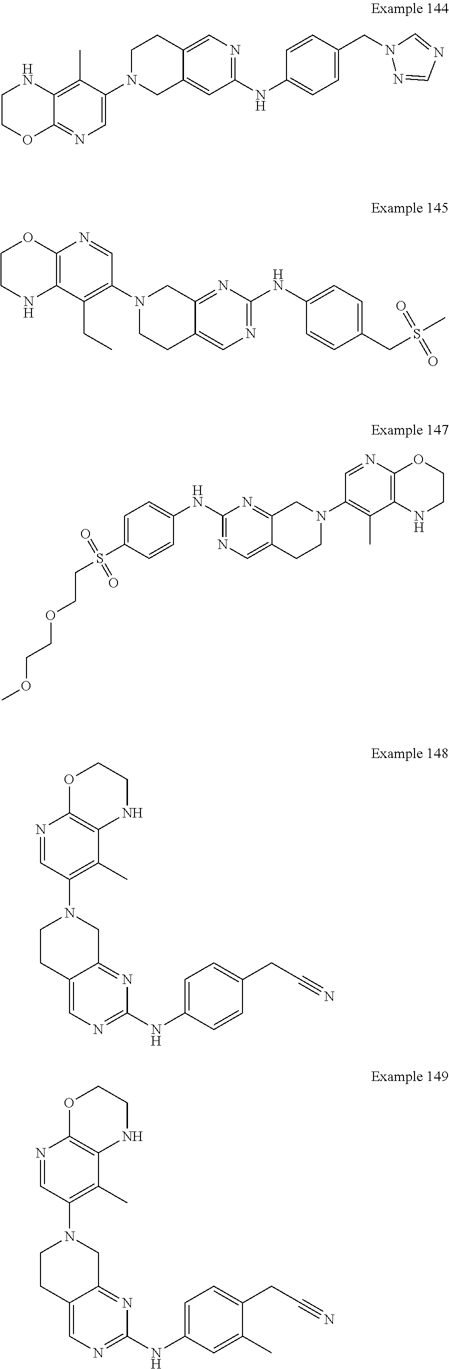

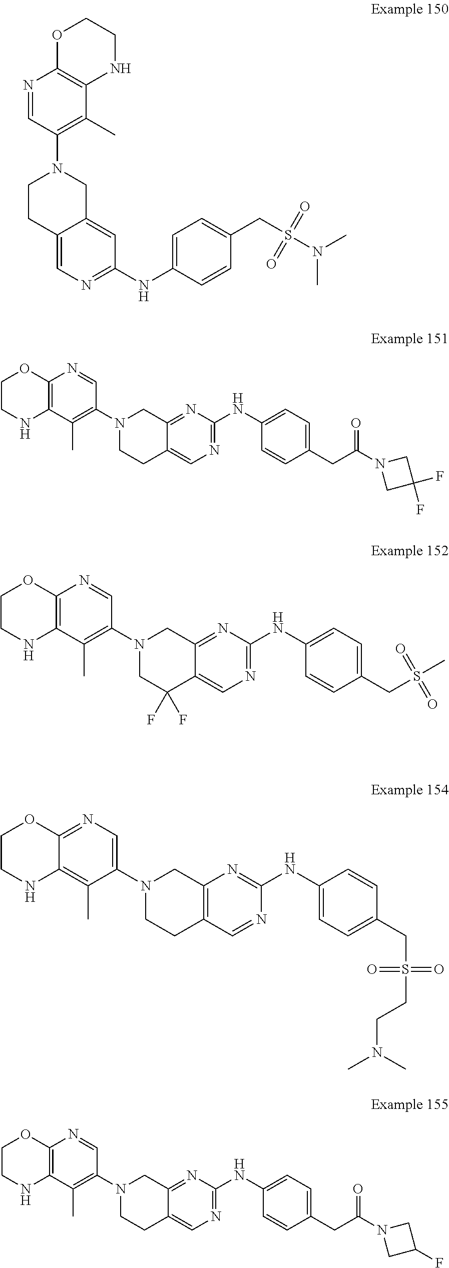 [SMILES: Cc1c(N2CCc3cnc(Nc4ccc(CS(=O)(=O)N(C)C)cc4)cc3C2)cnc2c1NCCO2 ; Cc1c(N2CCc3cnc(Nc4ccc(CC(=O)N5CC(F)(F)C5)cc4)nc3C2)cnc2c1NCCO2 ; Cc1c(N2Cc3nc(Nc4ccc(CS(C)(=O)=O)cc4)ncc3C(F)(F)C2)cnc2c1NCCO2 ; Cc1c(N2CCc3cnc(Nc4ccc(CS(=O)(=O)CCN(C)C)cc4)nc3C2)cnc2c1NCCO2 ; Cc1c(N2CCc3cnc(Nc4ccc(CC(=O)N5CC(F)C5)cc4)nc3C2)cnc2c1NCCO2]

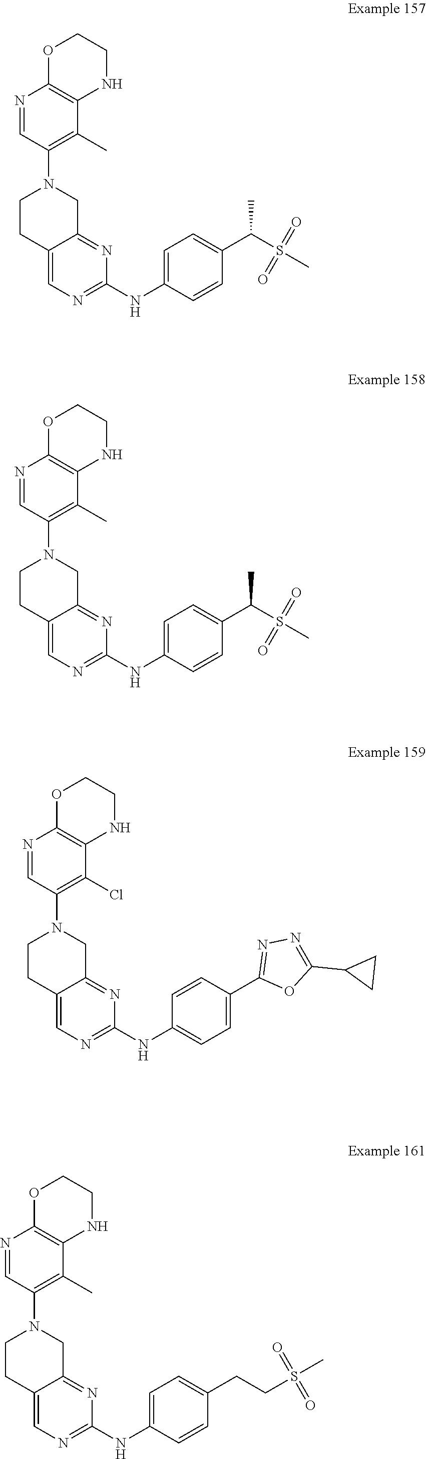 [SMILES: Cc1c(N2CCc3cnc(Nc4ccc([C@H](C)S(C)(=O)=O)cc4)nc3C2)cnc2c1NCCO2 ; Cc1c(N2CCc3cnc(Nc4ccc([C@@H](C)S(C)(=O)=O)cc4)nc3C2)cnc2c1NCCO2 ; Clc1c(N2CCc3cnc(Nc4ccc(-c5nnc(C6CC6)o5)cc4)nc3C2)cnc2c1NCCO2 ; Cc1c(N2CCc3cnc(Nc4ccc(CCS(C)(=O)=O)cc4)nc3C2)cnc2c1NCCO2]

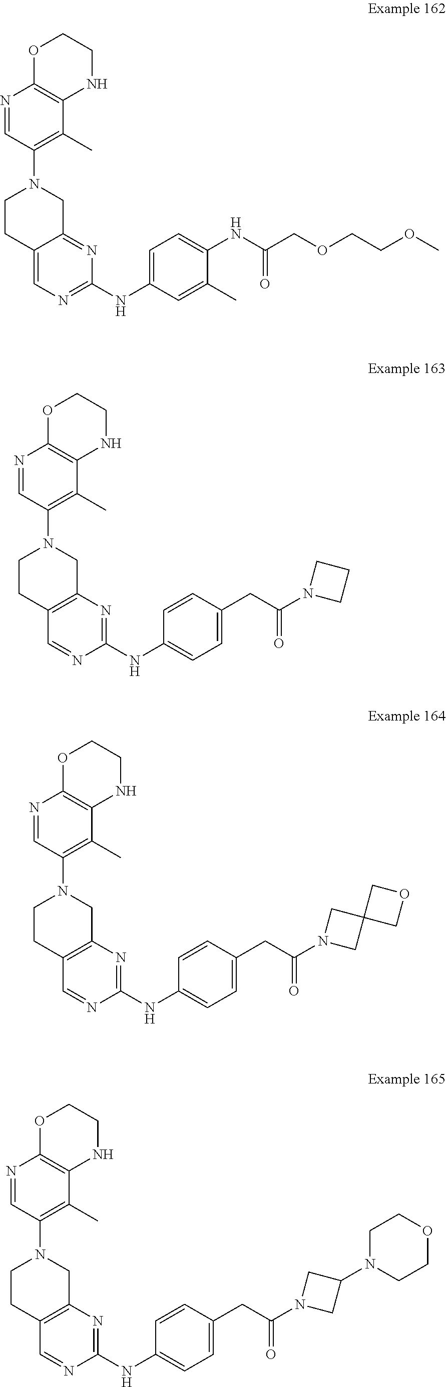 [SMILES: COCCOCC(=O)Nc1ccc(Nc2ncc3c(n2)CN(c2cnc4c(c2C)NCCO4)CC3)cc1C ; Cc1c(N2CCc3cnc(Nc4ccc(CC(=O)N5CCC5)cc4)nc3C2)cnc2c1NCCO2 ; Cc1c(N2CCc3cnc(Nc4ccc(CC(=O)N5CC6(COC6)C5)cc4)nc3C2)cnc2c1NCCO2 ; Cc1c(N2CCc3cnc(Nc4ccc(CC(=O)N5CC(N6CCOCC6)C5)cc4)nc3C2)cnc2c1NCCO2]

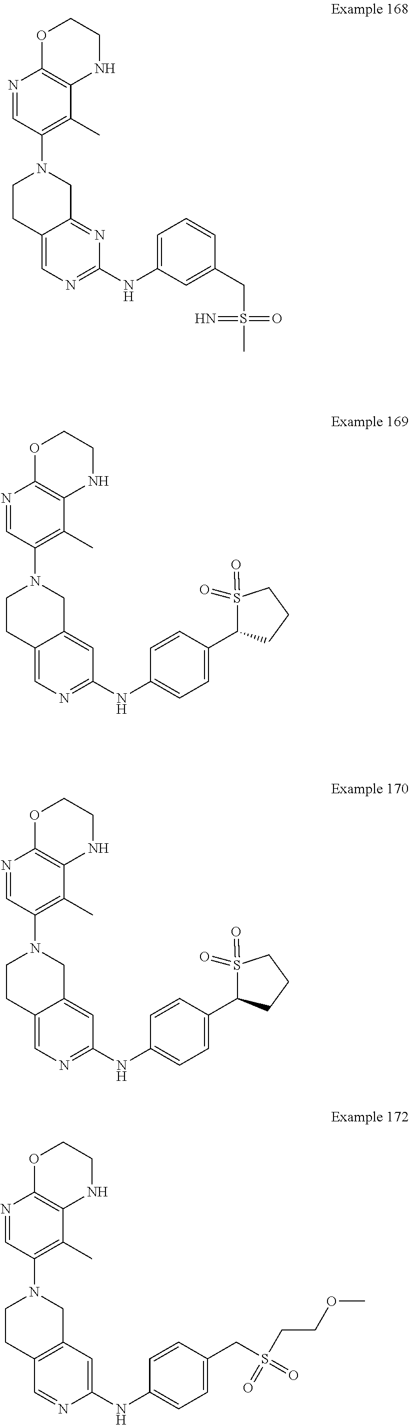 [SMILES: Cc1c(N2CCc3cnc(Nc4cccc(CS(C)(=N)=O)c4)nc3C2)cnc2c1NCCO2 ; Cc1c(N2CCc3cnc(Nc4ccc([C@H]5CCCS5(=O)=O)cc4)cc3C2)cnc2c1NCCO2 ; Cc1c(N2CCc3cnc(Nc4ccc([C@@H]5CCCS5(=O)=O)cc4)cc3C2)cnc2c1NCCO2 ; COCCS(=O)(=O)Cc1ccc(Nc2cc3c(cn2)CCN(c2cnc4c(c2C)NCCO4)C3)cc1]

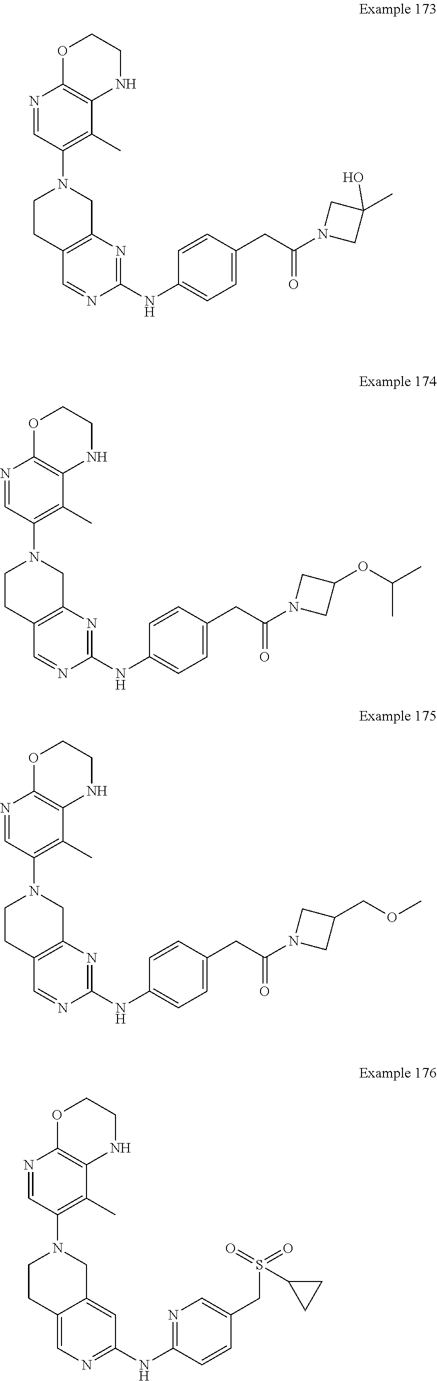 [SMILES: Cc1c(N2CCc3cnc(Nc4ccc(CC(=O)N5CC(C)(O)C5)cc4)nc3C2)cnc2c1NCCO2 ; Cc1c(N2CCc3cnc(Nc4ccc(CC(=O)N5CC(OC(C)C)C5)cc4)nc3C2)cnc2c1NCCO2 ; COCC1CN(C(=O)Cc2ccc(Nc3ncc4c(n3)CN(c3cnc5c(c3C)NCCO5)CC4)cc2)C1 ; Cc1c(N2CCc3cnc(Nc4ccc(CS(=O)(=O)C5CC5)cn4)cc3C2)cnc2c1NCCO2]

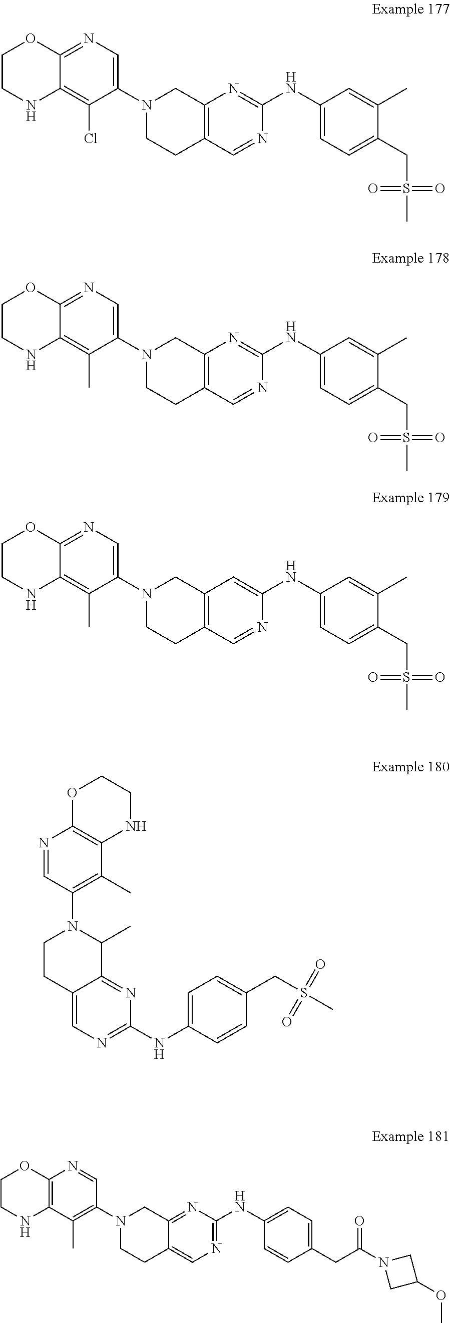 [SMILES: Cc1cc(Nc2ncc3c(n2)CN(c2cnc4c(c2Cl)NCCO4)CC3)ccc1CS(C)(=O)=O ; Cc1cc(Nc2ncc3c(n2)CN(c2cnc4c(c2C)NCCO4)CC3)ccc1CS(C)(=O)=O ; Cc1cc(Nc2cc3c(cn2)CCN(c2cnc4c(c2C)NCCO4)C3)ccc1CS(C)(=O)=O ; Cc1c(N2CCc3cnc(Nc4ccc(CS(C)(=O)=O)cc4)nc3C2C)cnc2c1NCCO2 ; COC1CN(C(=O)Cc2ccc(Nc3ncc4c(n3)CN(c3cnc5c(c3C)NCCO5)CC4)cc2)C1]

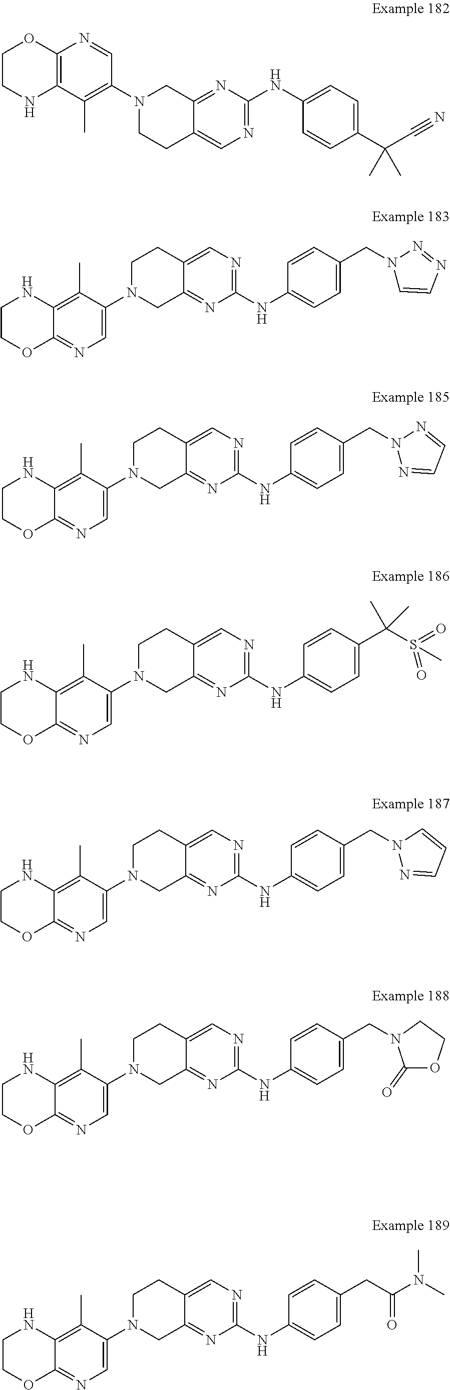

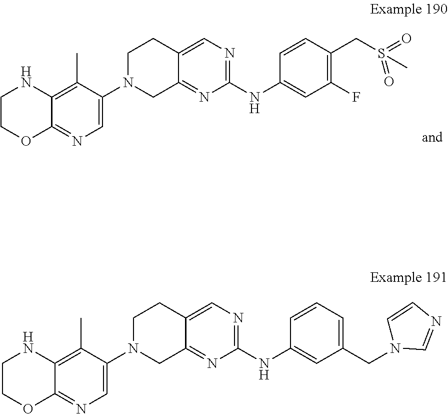 [SMILES: Cc1c(N2CCc3cnc(Nc4ccc(CS(C)(=O)=O)c(F)c4)nc3C2)cnc2c1NCCO2 ; Cc1c(N2CCc3cnc(Nc4cccc(Cn5ccnc5)c4)nc3C2)cnc2c1NCCO2]

and pharmaceutically acceptable salts thereof is provided.

In an embodiment a compound of the Formula II selected from the group consisting of

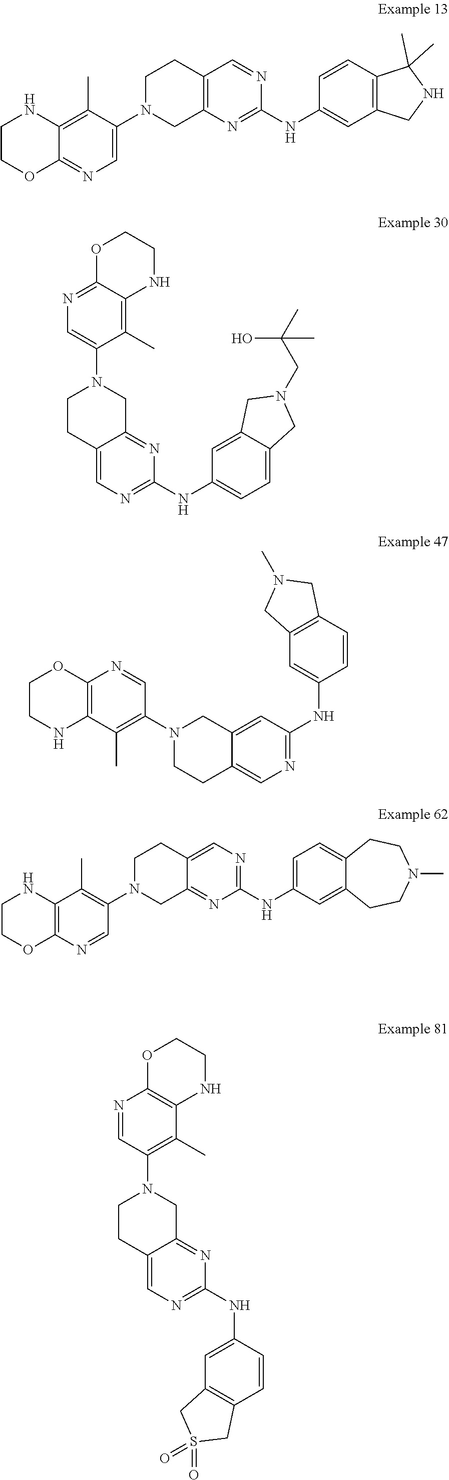 [SMILES: Cc1c(N2CCc3cnc(Nc4ccc5c(c4)CNC5(C)C)nc3C2)cnc2c1NCCO2 ; Cc1c(N2CCc3cnc(Nc4ccc5c(c4)CN(CC(C)(C)O)C5)nc3C2)cnc2c1NCCO2 ; Cc1c(N2CCc3cnc(Nc4ccc5c(c4)CN(C)C5)cc3C2)cnc2c1NCCO2 ; Cc1c(N2CCc3cnc(Nc4ccc5c(c4)CCN(C)CC5)nc3C2)cnc2c1NCCO2 ; Cc1c(N2CCc3cnc(Nc4ccc5c(c4)CS(=O)(=O)C5)nc3C2)cnc2c1NCCO2]

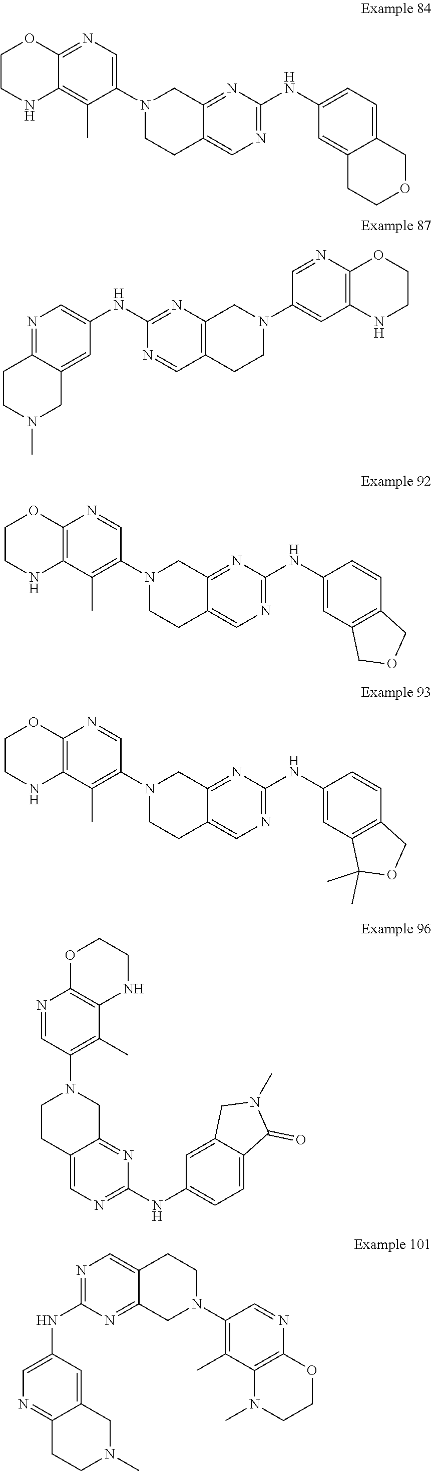 [SMILES: Cc1c(N2CCc3cnc(Nc4ccc5c(c4)CCOC5)nc3C2)cnc2c1NCCO2 ; CN1CCc2ncc(Nc3ncc4c(n3)CN(c3cnc5c(c3)NCCO5)CC4)cc2C1 ; Cc1c(N2CCc3cnc(Nc4ccc5c(c4)COC5)nc3C2)cnc2c1NCCO2 ; Cc1c(N2CCc3cnc(Nc4ccc5c(c4)C(C)(C)OC5)nc3C2)cnc2c1NCCO2 ; Cc1c(N2CCc3cnc(Nc4ccc5c(c4)CN(C)C5=O)nc3C2)cnc2c1NCCO2 ; Cc1c(N2CCc3cnc(Nc4cnc5c(c4)CN(C)CC5)nc3C2)cnc2c1N(C)CCO2]

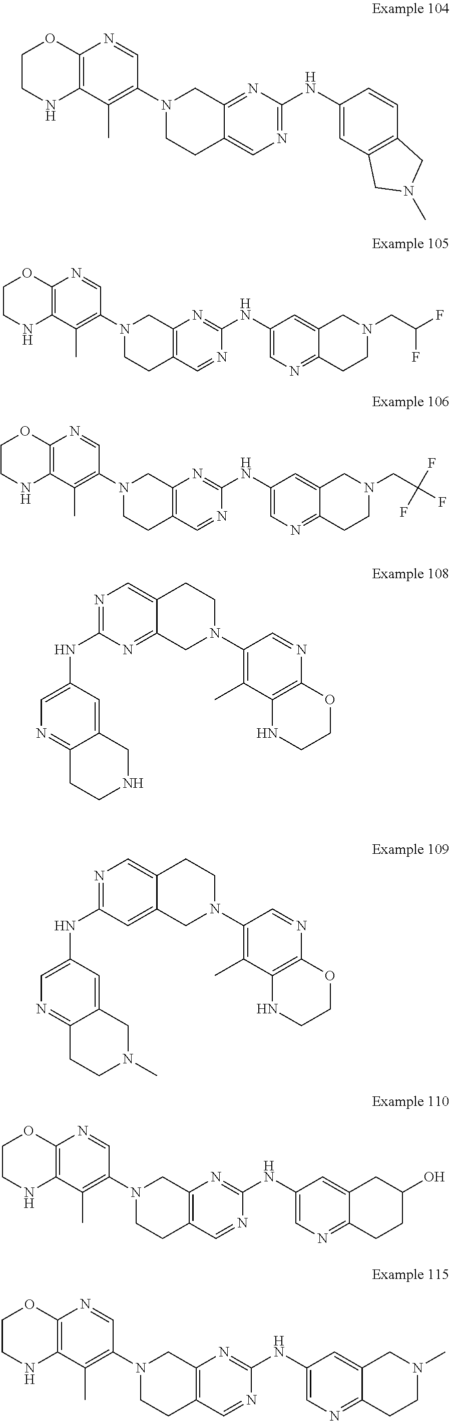

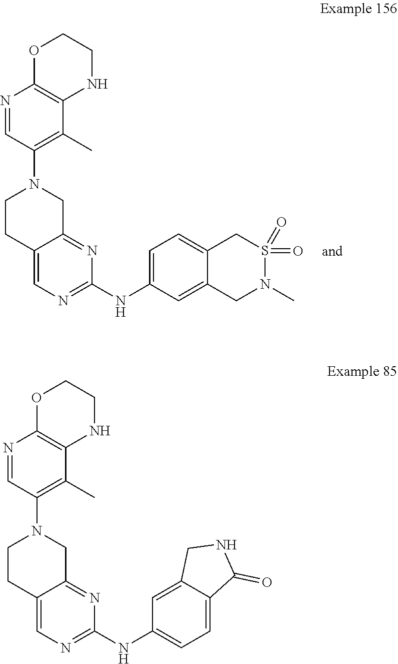

and pharmaceutically acceptable salts thereof is provided.

In an embodiment a compound of the Formula III selected from the group consisting of

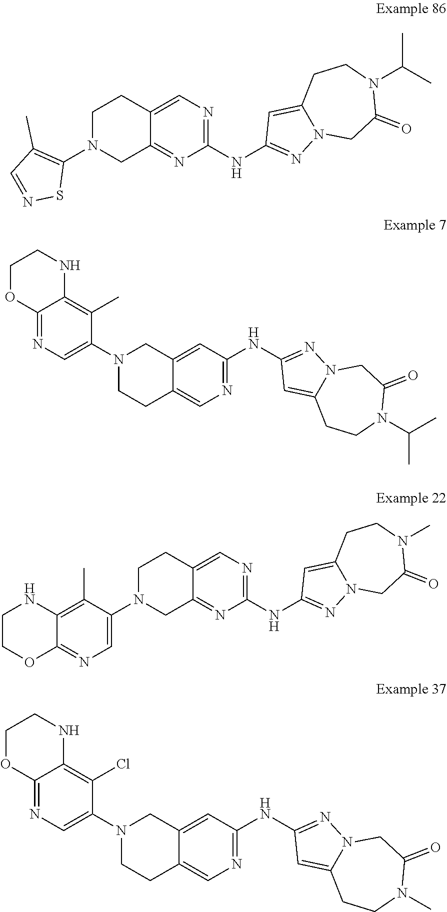 [SMILES: Cc1cnsc1N1CCc2cnc(Nc3cc4n(n3)CC(=O)N(C(C)C)CC4)nc2C1 ; Cc1c(N2CCc3cnc(Nc4cc5n(n4)CC(=O)N(C(C)C)CC5)cc3C2)cnc2c1NCCO2 ; Cc1c(N2CCc3cnc(Nc4cc5n(n4)CC(=O)N(C)CC5)nc3C2)cnc2c1NCCO2 ; CN1CCc2cc(Nc3cc4c(cn3)CCN(c3cnc5c(c3Cl)NCCO5)C4)nn2CC1=O]

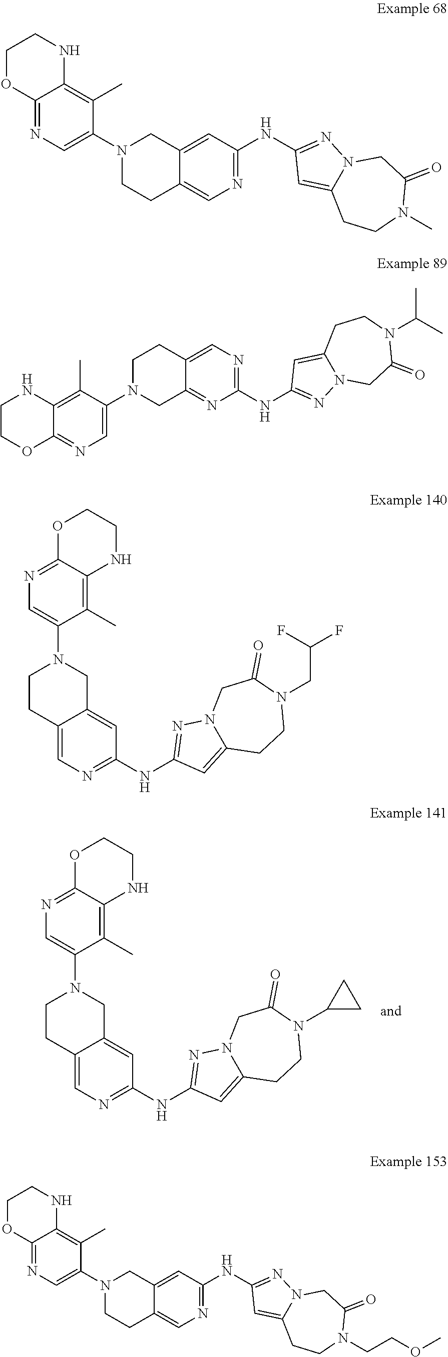 [SMILES: Cc1c(N2CCc3cnc(Nc4cc5n(n4)CC(=O)N(C)CC5)cc3C2)cnc2c1NCCO2 ; Cc1c(N2CCc3cnc(Nc4cc5n(n4)CC(=O)N(C(C)C)CC5)nc3C2)cnc2c1NCCO2 ; Cc1c(N2CCc3cnc(Nc4cc5n(n4)CC(=O)N(CC(F)F)CC5)cc3C2)cnc2c1NCCO2 ; Cc1c(N2CCc3cnc(Nc4cc5n(n4)CC(=O)N(C4CC4)CC5)cc3C2)cnc2c1NCCO2 ; COCCN1CCc2cc(Nc3cc4c(cn3)CCN(c3cnc5c(c3C)NCCO5)C4)nn2CC1=O]

and pharmaceutically acceptable salts thereof is provided.

In an embodiment a compound of the Formula IV selected from the group consisting of

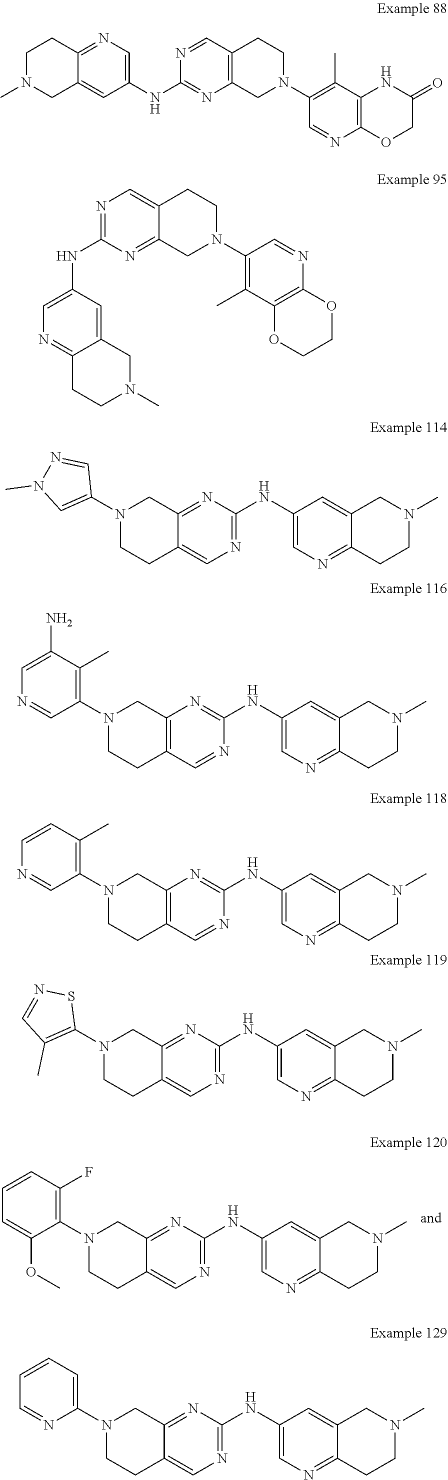

and pharmaceutically acceptable salts thereof is provided.

In an embodiment a compound of the Formula V selected from the group consisting of

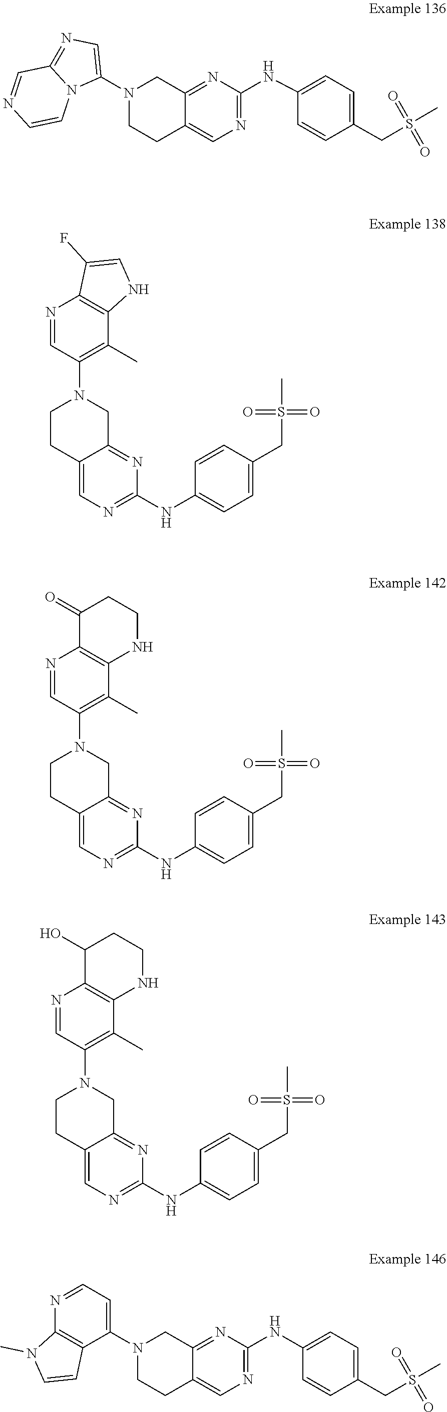

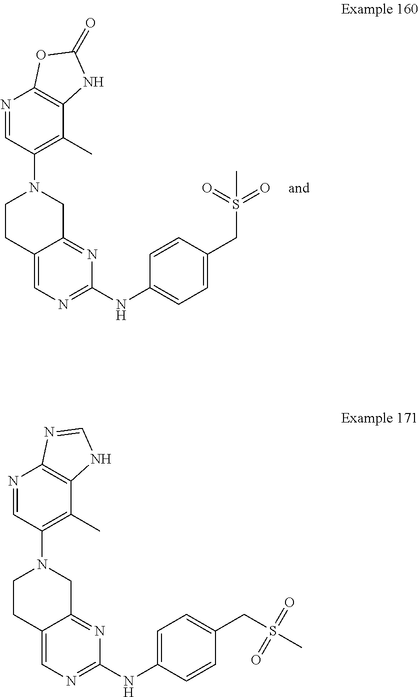

and pharmaceutically acceptable salts thereof is provided.

In an embodiment, a pharmaceutical composition comprises a compound of Formula I, II, III, IV or V as defined above and a pharmaceutically acceptable adjuvant, carrier, or vehicle.

In an embodiment, a method is provided in which a therapeutically effective amount of the compound having the structure of Formula I, II, III, IV or V or a pharmaceutically acceptable salt thereof as defined above is administered to a patient having an HPK1-mediated disorder.

In an embodiment, a method is provided in which a therapeutically effective amount of the pharmaceutical composition that contains a compound of Formula I, II, III, IV or V and a pharmaceutically acceptable adjuvant, carrier, or vehicle, is administered to a patient having an HPK1-mediated disorder. The HPK1-mediated disorder may be cancer or an autoimmune and/or inflammatory disease.

The HPK1-mediated disorder may be a cancer. The cancer may be one or more of cancer of the breast, bladder, bone, brain, central and peripheral nervous system, colon, endocrine glands, esophagus, endometrium, germ cells, head and neck, kidney, liver, lung, larynx and hypopharynx, ovary, pancreas, prostate, rectum, renal, small intestine, soft tissue, testis, stomach, skin, ureter, vagina, and vulva.

The HPK1-mediated disorder may be an autoimmune disease. The autoimmune disease may be one or more of rheumatoid arthritis (RA), autoimmune pancreatitis (AIP), systemic lupus erythematosus (SLE), type I diabetes mellitus, multiple sclerosis (MS), antiphospholipid syndrome (APS), sclerosing cholangitis, systemic onset arthritis, irritable bowel disease (IBD), scleroderma, Sjogren's disease, vitiligo, polymyositis, Pemphigus vulgaris, Pemphigus foliaceus, inflammatory bowel disease including Crohn's disease and ulcerative colitis, autoimmune hepatitis, hypopituitarism, graft-versus-host disease (GvHD), autoimmune skin diseases, uveitis, pernicious anemia, and hypoparathyroidism. Autoimmune diseases may also include, without limitation, polyangiitis overlap syndrome, Kawasaki's disease, sarcoidosis, glomerulonephritis, and cryopathy.

A compound of Formula I, II, III, IV or V or pharmaceutical composition thereof may be used to treat an inflammatory disorder and/or the immune response is associated with an inflammatory disorder. The inflammatory disorder may be one or more of non-rheumatoid arthritis, kidney fibrosis, and liver fibrosis. The inflammatory disorder may be an interface dermatitis. The inflammatory disorder may be a skin disorder such as atopic dermatitis (eczema); a sterile inflammatory condition such as drug-induced liver and/or pancreas inflammation; an inflammatory liver disorder; and/or an inflammatory pancreatic disorder.

A therapeutically effective amount of the compound or pharmaceutical composition may be in a range consisting of 0.1 to 100 mg/kg of body weight of the patient, 0.1 to 50 mg/kg of body weight of the patient, 0.5 to 50 mg/kg of body weight of the patient, 1 to 20 mg/kg of body weight of the patient, 5 to 20 mg/kg of body weight of the patient, 10 to 20 mg/kg of body weight of the patient, 10 to 50 mg/kg of body weight of the patient, and 10 to 100 mg/kg of body weight of the patient. The compound or pharmaceutical composition may be administered to the patient continuously, multiple times daily, once daily, once every other day, weekly, bi-weekly, monthly, or bi-monthly. The compound or pharmaceutical composition may be administered orally, parenterally, by inhalation spray, topically, rectally, nasally, buccally, vaginally, or via an implanted reservoir. The compound may be administered subcutaneously, intravenously, intramuscularly, intra-articularly, intra-synovially, intrasternally, intrathecally, intrahepaticly, intralesionally, and by intracranial injection or infusion technique.

In an embodiment, a kit is provided that includes a therapeutically effective amount of a compound of Formula I, II, III, IV or V as defined above or a physiologically acceptable salt or prodrug thereof; and instructions for use of the compound.

Additional features, advantages, and embodiments of the disclosed subject matter may be set forth or apparent from consideration of the following detailed description and claims. Moreover, it is to be understood that both the foregoing summary and the following detailed description are exemplary and are intended to provide further explanation without limiting the scope of the claims.

### DETAILED DESCRIPTION OF THE INVENTION

Provided herein is the chemical structure of the general Formula I, II, III, IV or V or a prodrug or pharmaceutically acceptable salt of any of the foregoing including mixtures thereof in all ratios, preferably for use in the treatment and/or cancer, inflammatory and/or autoimmune diseases. The compounds of Formula I, II, III, IV or V can inhibit HPK1 activity. The compounds disclosed herein have the general Formula (I):

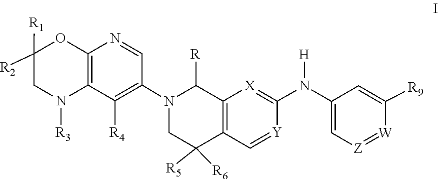

wherein:
R₁ and R₂ are each independently selected from the group consisting of hydrogen and C₁-C₆ alkyl, C₃-C₆ cycloalkyl and C₁-C₆ haloalkyl;
R₁ and R₂ together with the carbon to which they are attached can be taken together to form a C₃-C₆ carbocyclic ring;
R₃ is selected from the group consisting of hydrogen and C₁-C₆ alkyl;
R₄ is selected from the group consisting of hydrogen, halogen, C₁-C₆ alkyl, C₃-C₆ cycloalkyl and C₁-C₆ haloalkyl;
R₅ and R₆ are each independently selected from the group consisting of hydrogen, halogen, C₁-C₆ alkyl, C₃-C₆ cycloalkyl and C₁-C₆ haloalkyl;
R₇ is selected from the group consisting of hydrogen and C₁-C₆ alkyl;
X is selected from the group consisting of N and CH;
Y is selected from the group consisting of N and CH;
W is selected from the group consisting of N and CR₈;
Z is selected from the group consisting of N and CH;
R₈ and R₉ are independently selected from the group consisting of hydrogen, halogen, optionally substituted C₁-C₆ alkyl, optionally substituted C₃-C₆ cycloalkyl, C₁-C₆ haloalkyl, optionally substituted C₂-C₈ heterocyclic, optionally substituted heteroaromatic, optionally substituted C₆-C₁₄ aromatic, —(CH₂)ₘ—NR′R″, —NR′—(CH₂)ₘ—NR′R″, —(CH₂)ₙ—OR₁₁, —O—(CH₂)ₙ—OR₁₁;

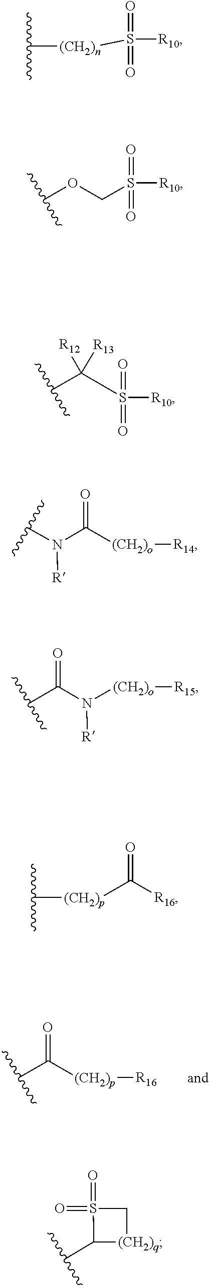

R₁₀ is selected independently for each occurrence from the group consisting C₁-C₆ alkyl, —NR′R″, —(CH₂)ₚ—OR₁₁, —(CH₂)ₙ—O—(CH₂)ₙ—OR₁₁ and —(CH₂)ₘ—NR′R″;
R′ and R″ are selected independently for each occurrence from the group consisting of hydrogen, C₁-C₆ alkyl and C₃-C₆ cycloalkyl;
R₁₂ and R₁₃ are independently selected for each occurrence from the group consisting of hydrogen and C₁-C₆ alkyl; R₁₂ and R₁₃ together with the carbon to which they are attached can be taken together to form a C₃-C₆ carbocyclic ring;
R₁₁, R₁₄, R₁₅ and R₁₆ are independently selected for each occurrence from the group consisting of hydrogen, C₁-C₆ alkyl, optionally substituted C₂-C₈ heterocyclic, optionally substituted heteroaromatic, optionally substituted C₆-C₁₄ aromatic; m, n, o, p and q are each independently selected from the group consisting of 1, 2 3, 4, 5 and 6;
or enantiomers or diastereomers or mixtures thereof or pharmaceutically acceptable salts thereof:
the general Formula II:

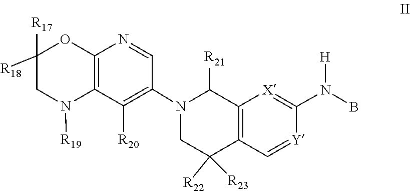

wherein:
B is selected from the group consisting of

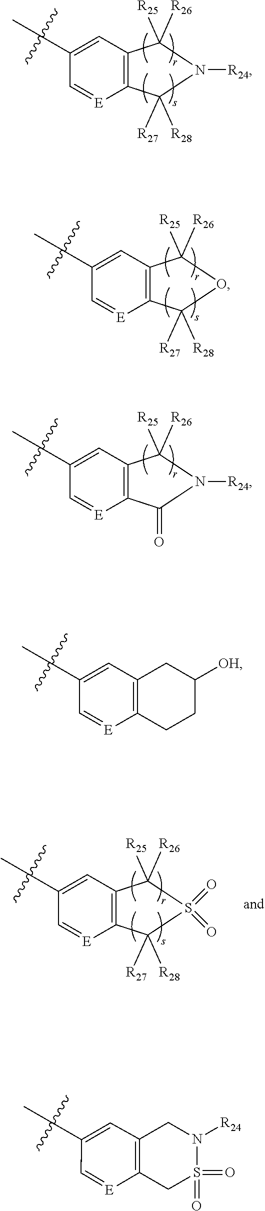

R₁₇ and R₁₈ are each independently selected from the group consisting of hydrogen and C₁-C₆ alkyl, C₃-C₆ cycloalkyl and C₁-C₆ haloalkyl;
R₁₇ and R₁₈ together with the carbon to which they are attached can be taken together to form a C₃-C₆ carbocyclic ring;
R₁₉ is selected from the group consisting of hydrogen and C₁-C₆ alkyl;
R₂₀ is selected from the group consisting of hydrogen, halogen, C₁-C₆ alkyl, C₃-C₆ cycloalkyl and C₁-C₆ haloalkyl;
R₂₁ is selected from the group consisting of hydrogen and C₁-C₆ alkyl;
R₂₂ and R₂₃ are each independently selected from the group consisting of hydrogen, halogen, C₁-C₆ alkyl, C₃-C₆ cycloalkyl and C₁-C₆ haloalkyl;
R₂₄ is selected from the group consisting of hydrogen, optionally substituted C₁-C₆ alkyl, optionally substituted C₃-C₆ cycloalkyl and C₁-C₆ haloalkyl;
R₂₅, R₂₆, R₂₇ and are R₂₈ are independently selected for each occurrence from the group consisting of hydrogen and C₁-C₆ alkyl;
X′ is selected from the group consisting of N and CH;
Y′ is selected from the group consisting of N and CH;
E is selected from the group consisting of N and CH
s and r are independently selected from the group consisting of 1, 2 and 3 or enantiomers or diastereomers or mixtures thereof or pharmaceutically acceptable salts thereof:
the general Formula III

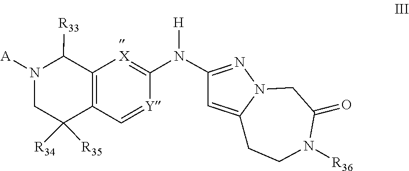

wherein:
A is selected from

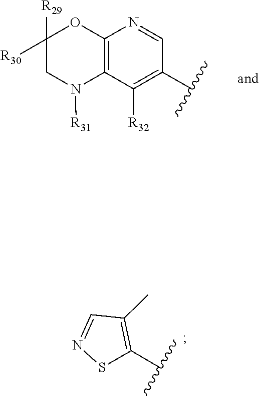

R₂₉ and R₃₀ are each independently selected from the group consisting of hydrogen and C₁-C₆ alkyl, C₃-C₆ cycloalkyl and C₁-C₆ haloalkyl;
R₂₉ and R₃₀ together with the carbon to which they are attached can be taken together to form a C₃-C₆ carbocyclic ring;
R₃₁ is selected from the group consisting of hydrogen and C₁-C₆ alkyl;
R₃₂ is selected from the group consisting of hydrogen, halogen, C₁-C₆ alkyl, C₃-C₆ cycloalkyl and C₁-C₆ haloalkyl;
R₃₃ is selected from the group consisting of hydrogen and C₁-C₆ alkyl;
R₃₄ and R₃₅ are each independently selected from the group consisting of hydrogen, halogen, C₁-C₆ alkyl, C₃-C₆ cycloalkyl and C₁-C₆ haloalkyl;
R₃₆ is selected from the group consisting of hydrogen, optionally substituted C₁-C₆ alkyl, optionally substituted C₃-C₆ cycloalkyl, —(CH₂)ᵤ—OR₃₇ and C₁-C₆ haloalkyl;
R₃₇ is selected from the group consisting of, hydrogen, C₁-C₆ alkyl, C₃-C₆ cycloalkyl and C₁-C₆ haloalkyl;
u is selected from the group consisting of 1, 2, 3, 4, 5 and 6;
X″ is selected from the group consisting of N and CH;
Y″ is selected from the group consisting of N and CH; or enantiomers or diastereomers or mixtures thereof or pharmaceutically acceptable salts thereof:
the general Formula IV

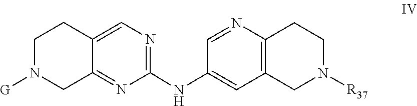

wherein:
G is selected from the group consisting of

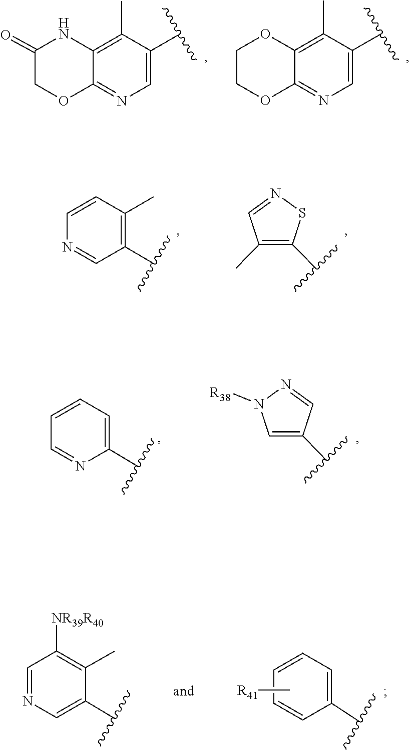

R₃₇, R₃₈, R₃₉ and R₄₀ are independently selected for each occurrence from the group consisting of hydrogen, C₁-C₆ alkyl, and C₃-C₆ cycloalkyl;
R₄₁ is independently selected for each position capable of substitution from the group consisting of hydrogen, halogen, —O—C₁-C₆ alkyl, C₁-C₆ haloalkyl, C₁-C₆ alkyl and C₃-C₆cycloalkyl; or enantiomers or diastereomers or mixtures thereof or pharmaceutically acceptable salts thereof:
and the general Formula V

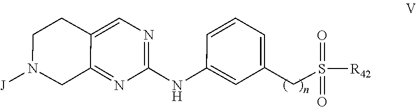

wherein:
J is selected from the group consisting of

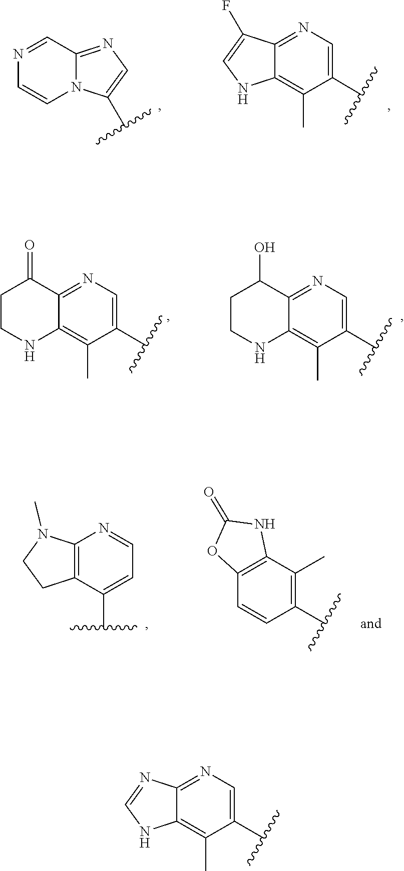

h is 1, 2 or 3
R₄₂ is selected from the group consisting of C₁-C₆ alkyl and C₃-C₆ cycloalkyl; or enantiomers or diastereomers or mixtures thereof or pharmaceutically acceptable salts thereof.

An alkyl can be a straight or branched alkyl group. Exemplary alkyl groups include, without limitation, methyl, ethyl, propyl, isopropyl, butyl, isobutyl, and tert-butyl. The number of carbon atoms in the alkyl is not particularly limited. In some embodiments, an alkyl may have a specified number of carbon atoms such as 1-6.

A halogen may refer to F, Cl, Br, or I. A haloalkyl may refer to one or more halogens bonded to an alkyl. An example of a haloalkyl may include CF₃.

A heterocyclyl is a univalent group formed by removing a hydrogen atom from any ring atom of a heterocyclic compound. It may include one or more heteroatoms. A heteroatom may refer to one or more of oxygen, sulfur, nitrogen, phosphorous, or any oxidized form thereof. A heterocycle, heterocyclyl, and heterocyclic ring may be used interchangeably and refer to a stable 5 to 7-membered monocyclic or 6 to 14-membered bicyclic heterocyclic moiety that is either saturated or partially unsaturated, and having, in addition to carbon atoms, one or more, preferably one to four, heteroatoms, as defined above. A heterocyclic ring can be attached to its pendant group at any heteroatom or carbon atom that results in a stable structure and any of the ring atoms can be optionally substituted. Examples of saturated or partially unsaturated heterocyclic rings include, without limitation, tetrahydrofuranyl, tetrahydrothiophenyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, oxazepinyl, thiazepinyl, and morpholinyl. A heterocyclyl may include groups in a heterocyclyl ring is fused to one or more aryl, heteroaryl, or cycloaliphatic rings, where the radical or point of attachment is on the heterocyclyl ring. A heterocyclyl group may be a mono- or bicyclic.

Partially unsaturated may refer to a ring moiety that includes at least one double or triple bond. It may encompass rings that have multiple sites of unsaturation, but it not intended to include aryl or heteroaryl moieties. The term unsaturated may refer to a moiety that has one or more units of unsaturation.

An aryl (aromatic) may refer to an aryl alone or a larger moiety such as an aralkyl, aralkoxy, and/or aryloxyalkyl. It may refer to monocyclic and bicyclic rings. At least one ring in the system may be aromatic. Each ring in the system may contain 3-7 members. Exemplary aryl groups include, without limitation, phenyl, biphenyl, naphthyl, anthracyl, and the like, which optionally includes one or more substituents. An aryl may also refer to a group in which an aromatic ring is fused to one or more non-aromatic rings such as indanyl, phthalimidyl, naphthimidyl, phenanthridinyl, tetrahydronaphthyl, and the like.

A heteroaryl (heteroaromatic) may refer to a heteroaryl alone or as a part of a larger moiety such as heteroaralkyl or heteroaralkoxy. It may refer to a group having 5-14 ring atoms, preferably 5 or 6 ring atoms. In addition to carbon atoms, the heteroaryl may include 1 to 5 heteroatoms as provided above. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. Heteroaryl may also include groups in which a heteroaromatic ring is fused to one or more aryl, cycloaliphatic, and/or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group is optionally mono- or bicyclic. A heteroaralkyl may refer to an alkyl group substituted by a heteroaryl, wherein the alkyl and heteroaryl portions independently are optionally substituted.

The term optionally substituted may refer to one or more hydrogens of the designated moiety being replaced with a suitable substituent. Unless otherwise indicated, optionally substituted group has a suitable substituent at each substitutable position of the group and when more than one position in any given structure is substituted with more than one substituent selected from the specified group, the substituent is either the same or different at every position. Typical substituents include, but are not limited to:

—F, —Cl, —Br, —I, deuterium, —OH, protected hydroxy, alkoxy, oxo, thiooxo, —NO₂, —CN, CF₃, N₃, —NH₂, protected amino, —NH alkyl, —NH alkenyl, —NH alkynyl, —NH cycloalkyl, —NH-aryl, —NH-heteroaryl, —NH-heterocyclic, -dialkylamino, -diarylamino, -diheteroarylamino, —O— alkyl, —O— alkenyl, —O— alkynyl, —O— cycloalkyl, —O-aryl, —O-heteroaryl, —O-heterocyclic, —C(O)— alkyl, —C(O)— alkenyl, —C(O)— alkynyl, —C(O)— carbocyclyl, —C(O)-aryl, —C(O)-heteroaryl, —C(O)-heterocyclyl, —CONH₂, —CONH— alkyl, —CONH— alkenyl, —CONH— alkynyl, —CONH-carbocyclyl, —CONH-aryl, —CONH-heteroaryl, —CONH-heterocyclyl, —OCO₂— alkyl, —OCO₂— alkenyl, —OCO₂— alkynyl, —OCO₂— carbocyclyl, —OCO₂-aryl, —OCO₂-heteroaryl, —OCO₂-heterocyclyl, —OCONH₂, —OCONH— alkyl, —OCONH— alkenyl, —OCONH— alkynyl, —OCONH— carbocyclyl, —OCONH— aryl, —OCONH— heteroaryl, —OCONH— heterocyclyl, —NHC(O)— alkyl, —NHC(O)— alkenyl, —NHC(O)— alkynyl, —NHC(O)— carbocyclyl, —NHC(O)-aryl, —NHC(O)-heteroaryl, —NHC(O)-heterocyclyl, —NHCO₂— alkyl, —NHCO₂— alkenyl, —NHCO₂— alkynyl, —NHCO₂— carbocyclyl, —NHCO₂— aryl, —NHCO₂— heteroaryl, —NHCO₂— heterocyclyl, —NHC(O)NH₂, —NHC(O)NH— alkyl, —NHC(O)NH— alkenyl, —NHC(O)NH— alkenyl, —NHC(O)NH— carbocyclyl, —NHC(O)NH-aryl, —NHC(O)NH-heteroaryl, —NHC(O)NH-heterocyclyl, NHC(S)NH₂, —NHC(S)NH— alkyl, —NHC(S)NH— alkenyl, —NHC(S)NH— alkynyl, —NHC(S)NH— carbocyclyl, —NHC(S)NH-aryl, —NHC(S)NH-heteroaryl, —NHC(S)NH-heterocyclyl, —NHC(NH)NH₂, —NHC(NH)NH— alkyl, —NHC(NH)NH— -alkenyl, —NHC(NH)NH— alkenyl, —NHC(NH)NH— carbocyclyl, —NHC(NH)NH-aryl, —NHC(NH)NH-heteroaryl, —NHC(NH)NH-heterocyclyl, —NHC(NH)— alkyl, —NHC(NH)— alkenyl, —NHC(NH)— alkenyl, —NHC(NH)— carbocyclyl, —NHC(NH)-aryl, —NHC(NH)-heteroaryl, —NHC(NH)-heterocyclyl, —C(NH)NH— alkyl, —C(NH)NH— alkenyl, —C(NH)NH— alkynyl, —C(NH)NH— carbocyclyl, —C(NH)NH-aryl, —C(NH)NH-heteroaryl, —C(NH)NH-heterocyclyl, —S(O)— alkyl, —S(O)— alkenyl, —S(O)— alkynyl, —S(O)— carbocyclyl, —S(O)-aryl, —S(O)-heteroaryl, —S(O)-heterocyclyl —SO₂NH₂, —SO₂NH-alkyl, —SO₂NH-alkenyl, —SO₂NH-alkynyl, —SO₂NH-carbocyclyl, —SO₂NH-aryl, —SO₂NH-heteroaryl, —SO₂NH-heterocyclyl, —NHSO₂— alkyl, —NHSO₂— alkenyl, —NHSO₂— alkynyl, —NHSO₂— carbocyclyl, —NHSO₂-aryl, —NHSO₂-heteroaryl, —NHSO₂— heterocyclyl, —CH₂NH₂, —CH₂SO₂CH₃, -mono-, di-, or tri-alkyl silyl, -alkyl, -alkenyl, -alkynyl, -aryl, -arylalkyl, -heteroaryl, -heteroarylalkyl, -heterocycloalkyl, -cycloalkyl, - carbocyclic, -heterocyclic, polyalkoxy alkyl, polyalkoxy, -methoxymethoxy, -methoxyethoxy, —SH, —S— alkyl, —S— alkenyl, —S— alkynyl, —S— carbocyclyl, —S-aryl, —S-heteroaryl, —S-heterocyclyl, or methylthiomethyl.

The term stable may refer to compounds that are not substantially altered when subjected to conditions to allow for their production or synthesis, detection, recovery, purification, and/or use as disclosed herein. A pharmaceutically acceptable salt may refer to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds disclosed herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid, or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts may be derived from appropriate bases include alkali metal, alkaline earth metal, and ammonium salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl sulfonate, and aryl sulfonate.

Unless otherwise stated, structures depicted herein are also meant to include all isomeric (e.g., enantiomeric, diastereomeric, and geometric (or conformational)) forms of the structure. For example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. In some instances, the enantiomeric excess is at least 50%, at least 60%, at least 70%, at least 85%, at least 90%, at least 95%, at least 97%, at least 99%, or 100%. Unless otherwise stated, all tautomeric forms of the compounds disclosed herein are within the scope of the invention. Since the pharmaceutical activity of the racemates or stereoisomers of the compounds according to the invention may differ, it may be desirable to use the enantiomer. In these cases, the end product or even the intermediates can be separated into enantiomeric compounds by chemical or physical measures known to the person skilled in the art or even employed as such in the synthesis.

Where tautomerism, e.g., keto-enol tautomerism, of compounds disclosed herein or their prodrugs may occur, the individual forms, e.g., the keto or the enol form, are claimed separately and together as mixtures in any ratio. The same applies for stereoisomers, e.g., enantiomers, cis/trans isomers, conformers, and the like. If desired, isomers can be separated by methods well known in the art, e.g., by liquid chromatography. The same applies for enantiomers, e.g., by using chiral stationary phases. Additionally, an enantiomer may be isolated by converting it into a diastereomer, i.e., coupling with an enantiomerically pure auxiliary compound, subsequent separation of the resulting diastereomer and cleavage of the auxiliary residue. Alternatively, any enantiomer of a compound disclosed herein may be obtained from stereoselective synthesis using optically pure starting materials.

It is also contemplated that compounds disclosed herein may include isotope-labeled forms thereof. An isotope-labeled form of a compound disclosed herein is identical to this compound apart from the fact that one or more atoms of the compound have been replaced by an atom or atoms having an atomic mass or mass number which differs from the atomic mass or mass number of the atom which usually occurs naturally. Examples of isotopes which are readily commercially available and can be incorporated into a compound of the Formula I, II, III, IV or V by well-known methods include, without limitation, isotopes of hydrogen, carbon, nitrogen, oxygen, phosphorus, fluorine, and chlorine, for example ²H, ³H, ¹³C, ¹⁴C, ¹⁵N, ¹⁸O, ¹⁷O, ³¹P, ³²P, ³⁵S, ¹⁸F, and ³⁶Cl, respectively. It is also contemplated that a compound of the Formula I, II, III, IV or V, a prodrug thereof, or a pharmaceutically acceptable salt of either which contains one or more of the above-mentioned isotopes and/or other isotopes of other atoms are embodiments of the present disclosure. An isotope-labeled compound of the Formula I, II, III, IV or V can be used in a number of beneficial ways. For example, an isotope-labeled compound of the Formula I, II, III, IV or V into which, for example, a radioisotope, such as ³H or ¹⁴C, has been incorporated, is suitable for medicament and/or substrate tissue distribution assays. These radioisotopes are particularly preferred due to their ease of preparation and excellent detectability. Incorporation of heavier isotopes, for example, deuterium (²H), into a compound of the Formula I, II, III, IV or V may have therapeutic advantages due to the higher metabolic stability of this isotope-labeled compound. Higher metabolic stability translates directly into an increased in vivo half-life or lower dosages. An isotope-labeled compound of the Formula I, II, III, IV or V can be adapted to the procedures disclosed in the synthesis schemes and the related description, in the example part and in the preparation part, disclosed herein, replacing a non-isotope-labeled reactant by a readily available isotope-labeled reactant.

The compounds disclosed herein can be in the form of a prodrug compound. A prodrug compound may refer to a derivative that is converted into a biologically active compound under physiological conditions in the living body, e.g., by oxidation, reduction, hydrolysis, or the like, each of which is carried out enzymatically, or without enzyme involvement. Examples of prodrugs are compounds, wherein the amino group in a compound is acylated, alkylated, or phosphorylated; wherein the hydroxyl group is acylated, alkylated, phosphorylated, or converted into borate; wherein the carboxyl group is esterified or amidated; or wherein a sulfhydryl group forms a disulfide bridge with a carrier molecule, e.g., a peptide, that delivers the drug selectively to a target and/or to the cytosol of a cell. These compounds can be produced from compounds disclosed herein according to well-known methods. Other examples of prodrugs are compounds, wherein the carboxylate in a compound is for example converted into an alkyl-, aryl-, choline-, amino, acyloxymethylester, or linolenoyl-ester.

According to an embodiment, a composition comprising a compound disclosed herein or a pharmaceutically acceptable derivative thereof and a pharmaceutically acceptable carrier, adjuvant, or vehicle. The amount of compound in compositions disclosed herein may be effective to measurably inhibit HPK1, or a mutant thereof, in a biological sample or in a patient. A therapeutically effective amount of the compound may be administered to a patient in need thereof. A patient or subject may refer to an animal, preferably a mammal, and even more preferably, a human. A biological sample may refer to, without limitation, cell cultures or extracts thereof; biopsied material obtained from a mammal or extracts thereof; and blood, saliva, urine, feces, semen, tears, or other body fluids or extracts thereof.

A pharmaceutically acceptable carrier, adjuvant, or vehicle may refer to a nontoxic carrier, an adjuvant, or a vehicle that does not destroy the pharmacological activity of the compound with which it is formulated. Pharmaceutically acceptable carriers, adjuvants, or vehicles that are used in the compositions disclosed herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. A pharmaceutically acceptable derivative means any nontoxic salt, ester, salt of an ester or other derivative of a compound disclosed herein that, upon administration to a recipient, is capable of providing, either directly or indirectly, a compound disclosed herein.

Compositions disclosed herein may be administered orally, parenterally, by inhalation spray, topically, rectally, nasally, buccally, vaginally, or via an implanted reservoir. Parenteral administration may refer to subcutaneous, intravenous, intramuscular, intra-articular, intra-synovial, intrasternal, intrathecal, intrahepatic, intralesional, and intracranial injection or infusion techniques. Preferably, the composition may be administered orally, intraperitoneally, or intravenously. Sterile injectable forms of the compositions disclosed herein include aqueous or oleaginous suspension. These suspensions are formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be used are water, Ringer's solution, and isotonic sodium chloride solution. In addition, sterile, fixed oils may be conventionally employed as a solvent or suspending medium.

Pharmaceutically acceptable compositions disclosed herein may be orally administered in any orally acceptable dosage form. Exemplary oral dosage forms are capsules, tablets, aqueous suspensions, or solutions. In the case of tablets for oral use, carriers commonly used include lactose and corn starch. Lubricating agents, such as magnesium stearate, are also typically added. For oral administration in a capsule form, useful diluents include lactose and dried corn starch. When aqueous suspensions are required for oral use, the active ingredient may be combined with emulsifying and suspending agents. If desired, certain sweetening, flavoring, and/or coloring agents may be optionally added. Most preferably, pharmaceutically acceptable compositions disclosed herein are formulated for oral administration. Such formulations may be administered with or without food.

An amount of the compounds disclosed herein that may be combined with the carrier materials to produce a composition in a single dosage form may vary depending upon the host treated, the particular mode of administration. Preferably, provided compositions may be formulated so that a dosage of between 0.01-100 mg/kg body weight/day of the compound can be administered to a patient receiving these compositions. In some instances, the dosage is between 0.1 to 100 mg/kg, 0.1 to 50 mg/kg, 0.5 to 50 mg/kg, 1 to 50 mg/kg, 1 to 20 mg/kg, 5 to 20 mg/kg, 10 to 20 mg/kg, 10-50 mg/kg of body weight of the patient, or 10-100 mg/kg of body weight of the patient or any other dosing range disclosed herein. In some instances, the compounds or compositions herein are administered continuously, multiple times daily, once daily, once every other day, weekly, bi-weekly, monthly, or bi-monthly. A therapeutically effective amount of a compound or composition herein may vary according to factors known in the art, but a dose of about 0.1 to 100 mg/kg, 0.1 to 50 mg/kg, 0.5-50 mg/kg, 1 to 50 mg/kg, 1 to 20 mg/kg, 5 to 20 mg/kg, 10 to 20 mg/kg, or 10-50 mg/kg of body weight of the patient, or 10-100 mg/kg of body weight of the patient or any other dosing range disclosed herein, may be therapeutically effective. A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound disclosed herein in the composition will also depend upon the particular compound in the composition.

The compounds of Formula I, II, III, IV or V disclosed herein can be administered before or following an onset of disease once or several times acting as therapy. The aforementioned compounds and medical products of the inventive use are particularly used for the therapeutic treatment. A therapeutically relevant effect relieves to some extent one or more symptoms of a disorder, or returns to normality, either partially or completely, one or more physiological or biochemical parameters associated with or causative of a disease or pathological condition. Monitoring is considered as a kind of treatment provided that the compounds are administered in distinct intervals, e.g., in order to boost the response and eradicate symptoms of the disease completely. Either the identical compound or different compounds can be applied. The methods disclosed herein can also be used to reduce the likelihood of developing a disorder or even prevent the initiation of disorders associated with HPK1 activity in advance or to treat the arising and continuing symptoms.

The host or patient can belong to any mammalian species, for example a primate species, particularly humans; rodents, including mice, rats, and hamsters; rabbits; horses, cows, dogs, cats, etc. Animal models are of interest for experimental investigations, providing a model for treatment of human disease.

The compounds disclosed herein may be useful as anticancer agents for cancers that are responsive to HPK1 inhibition. In certain embodiments, the cancer may include, without limitation, cancer of the breast, bladder, bone, brain, central and peripheral nervous system, colon, endocrine glands, esophagus, endometrium, germ cells, head and neck, kidney, liver, lung, larynx and hypopharynx, ovary, pancreas, prostate, rectum, renal, small intestine, soft tissue, testis, stomach, skin, ureter, vagina, and vulva. In some instances, the cancer is a mesothelioma, sarcoma, retinoblastoma, Wilms tumor, leukemia, lymphoma, non-Hodgkin disease, chronic and acute myeloid leukemia, acute lymphoblastic leukemia, Hodgkin disease, multiple myeloma, T-cell lymphoma, myelodysplastic syndrome, plasma cell neoplasia, and paraneoplastic syndromes.

The compounds of Formula I, II, III, IV or V may be used in a method for ameliorating symptoms associated with and/or treating unwanted immune activation, including, but not limited to, symptoms associated with and/or treatment of autoimmunity. The disclosed compounds may be useful to inhibit the immune response and thereby prevent or delay development of the autoimmune disease. The autoimmune disease may be characterized by joint pain, antinuclear antibody positivity, malar rash, or discoid rash. The autoimmune disease may be associated with the skin, muscle tissue, and/or connective tissue. In some embodiments, the autoimmune disease is not evidenced in the individual by skin, muscle tissue, and/or connective tissue symptoms. In some embodiments, the autoimmune disease is systemic. Autoimmune diseases include, without limitation, rheumatoid arthritis (RA), autoimmune pancreatitis (AIP), systemic lupus erythematosus (SLE), type I diabetes mellitus, multiple sclerosis (MS), antiphospholipid syndrome (APS), sclerosing cholangitis, systemic onset arthritis, irritable bowel disease (IBD), scleroderma, Sjogren's disease, vitiligo, polymyositis, Pemphigus vulgaris, Pemphigus foliaceus, inflammatory bowel disease including Crohn's disease and ulcerative colitis, autoimmune hepatitis, hypopituitarism, graft-versus-host disease (GvHD), autoimmune skin diseases, uveitis, pernicious anemia, and hypoparathyroidism. Autoimmune diseases may also include, without limitation, polyangiitis overlap syndrome, Kawasaki's disease, sarcoidosis, glomerulonephritis, and cryopathy.

In some embodiments, the disclosed compounds may be used to treat an inflammatory disorder and/or the immune response is associated with an inflammatory disorder. As used herein, the term “inflammatory disorder” may encompass autoimmune diseases, as well as inflammatory conditions without a known autoimmune component (e.g., atherosclerosis, asthma, etc.). In further aspects, inhibiting the immune response may alleviate or mitigate one or more symptoms of the inflammatory disorder. Inhibiting the immune response may be useful to treat the inflammatory disorder and/or prevent or delay development of the inflammatory disorder. The inflammatory disorder may include, without limitation, non-rheumatoid arthritis, kidney fibrosis, and liver fibrosis. The inflammatory disorder may be an interface dermatitis. The interface dermatitis may be one or more of lichen planus, lichenoid eruption, lichen planus-like keratosis, Lichen striatus, keratosis lichenoides chronica, erythema multiforme, fixed drug eruption, Pityriasis lichenoides, phototoxic dermatitis, radiation dermatitis, viral exanthems, dermatomyositis, secondary syphilis, lichen sclerosus et atrophicus, mycosis fungoides, bullous pemphigoid, Lichen aureus, porokeratosis, acrodermatitis chronicus atrophicans, and regressing melanoma. The inflammatory disorder may be a skin disorder such as atopic dermatitis (eczema); a sterile inflammatory condition such as drug-induced liver and/or pancreas inflammation; an inflammatory liver disorder; and/or an inflammatory pancreatic disorder.

Sensitivity of a given cancer to HPK1 inhibition can be assessed by, without limitation, measurement of a decrease in primary or metastatic tumor load (minor, partial, or complete regression), alterations in the hemogram, altered hormone or cytokine concentrations in the blood, inhibition of further increase of tumor load, stabilization of the disease in the patient, assessment of biomarkers or surrogate markers relevant for the disease, prolonged overall survival of a patient, prolonged time to disease progression of a patient, prolonged progression-free survival of a patient, prolonged disease-free survival of a patient, improved quality of life of a patient, or modulation of the co-morbidity of the disease (for example, but not limited to, pain, cachexia, mobilization, hospitalization, altered hemogram, weight loss, wound healing, fever). Significant improvements in the pharmacokinetic profiles of compounds of the Formula I, II, III, IV or V may thereby be obtained, and can be expressed quantitatively in terms of increases in the in vivo half-life (t/2), concentration at maximum therapeutic effect (Cmax) and area under the dose response curve (AUC).

In various embodiments, compounds of Formula I, II, III, IV or V and related formulae, exhibit an IC₅₀ for the inhibiting HPK1 of less than about 1000 nM, less than about 500 nM, less than 100 nM, less than 500 nM, or less than 1000 nM. In some embodiments, the compounds of formula (I), and related formulae exhibit an IC₅₀ for the inhibiting HPK1 of at least 1 nM, at least 10 nM, at least 100 nM, or at least 500 nM. In some embodiments, the range is a combination of these values.

Compounds of Formula I, II, III, IV or V and/or a pharmaceutically acceptable salt thereof can be employed as an intermediate for the preparation of further medicament active ingredients. The medicament is preferably prepared in a non-chemical manner, e.g., by combining the active ingredient with at least one solid, fluid and/or semi-fluid carrier or excipient, and optionally in conjunction with a single or more other active substances in an appropriate dosage form.

A medicament is provided herein that can include at least one compound disclosed herein or a prodrug or pharmaceutically acceptable salt thereof including mixtures thereof in all ratios. A medicament may refer to any agent in the field of medicine, which comprises one or more compounds of Formula I, II, III, IV or V or preparations thereof (e.g., a pharmaceutical composition or pharmaceutical formulation) and can be used in prophylaxis, therapy, follow-up or aftercare of patients who suffer from diseases, which are associated with HPK1 activity, in such a way that a pathogenic modification of their overall condition or of the condition of particular regions of the organism could establish at least temporarily.

In various embodiments, the active ingredient may be administered alone or in combination with other treatments. A synergistic effect may be achieved by using more than one compound in the pharmaceutical composition, i.e. the compound of Formula I, II, III, IV or V may be combined with at least another agent as active ingredient, which is either another compound of Formula I, II, III, IV or V or a compound of different structural scaffold. The active ingredients can be used either simultaneously or sequentially. In some embodiments, the HPK1 inhibitor compounds disclosed herein is administered simultaneously with one or more additional therapeutic agents. In some embodiments, sequential administration includes administering the HPK1 inhibitor or additional therapeutic agent followed within about any of one minute, five minutes, 30 minutes, one hour, five hours, 24 hours, 48 hours, or a week. In some embodiments, the HPK1 inhibitor is administered by the same route of administration as the additional therapeutic agent. In some embodiments, the HPK1 inhibitor may be administered by a different route of administration than the additional therapeutic agent.

A therapeutic agent may include, without limitation, an anti-inflammatory drug and/or one or more anti-tumor agents conventionally used in chemotherapy or targeted therapy. The compounds or compositions disclosed herein may be used as a monotherapy or may be combined with therapeutic agents. Examples of anti-tumor agents include, without limitation, platinum compounds such as carboplatin, cisplatin, picoplatin, and the like; alkylating agents such as altretamine, carmustine, chlorambucil, mitobronitol, apaziquone, palifosfamide, and the like; DNA altering agents such as bisantrene, decitabine, mitoxantrone, procarbazine, and the like; microtubule modifiers such as docetaxel, eribulin, paclitaxel, vinblastine, and the like; topoisomerase inhibitor such as etoposide, razoxane, topotecan, and the like; anticancer antibodies such as bleomycin, mitomycin C, and the like; antimetabolites such as capecitabine, cladribine, and the like; hormones or antagonists such as tamoxifen, dexamethasone, and the like; cytokines such as interferon and the like; antibodies such as pembrolizumab, nivolumab, ipilimumab, cetuximab, and the like.

A method for inhibiting abnormal cell growth in a mammal, preferably a human, or treating a cancer may include administering to the mammal an amount of a compound of Formula I, II, III, IV or V disclosed herein, or a prodrug or a pharmaceutically acceptable salt thereof, in combination with radiation therapy, wherein the amounts of the compound, salt, or prodrug, is in combination with the radiation therapy effective in inhibiting abnormal cell growth or treating the cancer or symptoms thereof in the mammal. Techniques for administering radiation therapy are known in the art, and these techniques can be used in the combination therapy described herein.

As used herein, the terms “treatment”, “treat”, and “treating” may refer to reversing, alleviating, delaying the onset of, or inhibiting the progress of a disease or disorder, or one or more symptoms thereof, as described herein. In some embodiments, treatment is administered after one or more symptoms have developed. In other embodiments, treatment is administered in the absence of symptoms. For example, treatment is administered to a susceptible individual prior to the onset of symptoms (e.g., in light of a history of symptoms and/or in light of genetic or other susceptibility factors). Treatment is also continued after symptoms have resolved, for example to prevent or delay their recurrence.

Disclosed herein is a kit that includes separate packs of a therapeutically effective amount of a compound disclosed herein or a physiologically acceptable salt, or prodrug thereof, and optionally, a therapeutically effective amount of a therapeutic agent. The kit may include suitable containers, such as boxes, individual bottles, bags, or ampoules as well as instructions for using or applying the kit. The kit may, for example, contain separate ampoules, each containing a therapeutically effective amount of a compound disclosed herein and/or pharmaceutically usable derivatives, solvates and stereoisomers thereof, including mixtures thereof in all ratios, and an effective amount of a further therapeutic agent in dissolved or lyophilized form.

### Experimental Procedures

As depicted in the Examples below, in certain exemplary embodiments, compounds are prepared according to the following general procedures. It will be appreciated that, although the general methods depict the synthesis of certain compounds, the following general methods, and other methods known to one of ordinary skill in the art, can be applied to all compounds and subclasses and species of each of these compounds, as described herein.

The symbols and conventions used in the following descriptions of processes, schemes, and examples are consistent with those used in the contemporary scientific literature, for example, the Journal of the American Chemical Society or the Journal of Biological Chemistry.

Unless otherwise indicated, all temperatures are expressed in ° C. (degrees Centigrade).

All solvents used were commercially available and were used without further purification. Reactions were typically run using anhydrous solvents under an inert atmosphere of nitrogen. Flash column chromatography was generally carried out using Silica gel 60 (0.035-0.070 mm particle size).

All NMR experiments were recorded either on a Bruker Mercury Plus 400 NMR Spectrometer equipped with a Bruker 400 BBFO probe at 400 MHz for proton NMR, or on a Bruker Mercury Plus 300 NMR Spectrometer equipped with a Bruker 300 BBFO probe at 300 MHz for proton NMR, or on a Bruker Avance III 400 NMR Spectrometer equipped with a Bruker PABBO BB-1H/D Z GRD probe at 400 MHz for proton NMR. Most deuterated solvents contained typically 0.03% to 0.05% v/v tetramethylsilane, which was used as the reference signal (set at d 0.00 for both 1H and 13C). In cases where the deuterated solvents did not contain tetramethylsilane, the residual non-deuterated solvent peaks were used as a reference signal, as per published guidelines (J. Org. Chem., vol. 62, No. 21, 1997).

LC-MS analyses were performed one either one of the two following instruments:

1. SHIMADZU LC-MS machine consisting of an UFLC 20-AD system and LCMS 2020 MS detector. The column used was a Shim-pack XR-ODS, 2.2 μm, 3.0×50 mm. A linear gradient was applied, starting at 95% A (A: 0.05% trifluoroacetic acid (TFA) in water) and ending at 100% B (B: 0.05% TFA in acetonitrile (ACN)) over 2.2 min. with a total run time of 3.6 min. The column temperature was at 40° C. with the flow rate at 1.0 mL/min. The Diode Array detector was scanned from 200-400 nm. The mass spectrometer was equipped with an electro spray ion source (ES) operated in a positive or negative mode. The mass spectrometer was scanned between m/z 90-900 with a scan time of 0.6 s.

2. Agilent 1200 Series mass spectrometers from Agilent Technologies, using either Atmospheric Chemical Ionization (APCI) or Electrospray Ionization (ESI). Diode Array detector was scanned from 200-400 nm. The mass spectrometer was scanned between m/z 90-900 with a scan time of 0.6 s. Column: XBridge C8, 3.5 μm, 4.6×50 mm; Solvent A: water+0.1% TFA; Solvent B: ACN+0.1% TFA; Flow: 2 mL/min.; Gradient: 0 min: 5% B, 8 min.: 100% B, 8.1 min.: 100% B, 8.5 min.: 5% B, 10 min. 5% B or a LC/MS waters ZMD (ESI).

HPLC data were either obtained from the SHIMAZU LC-MS machine or using Agilent 1100 series HPLC from Agilent technologies using a column (XBridge C8, 3.5 μm, 4.6×50 mm) and two mobile phases (mobile phase A: water+0.1% TFA; mobile phase B: ACN+0.1% TFA). The flow rate was 2 mL/min. The gradient method was: 0 min.: 5% B; 8 min.: 100% B; 8.1 min.: 100% B; 8.5 min.: 5% B; 10 min. 5% B, unless otherwise indicated.

In general, the compounds according to Formula I, II, III, IV or V and related formulae described herein can be prepared from readily available starting materials. If such starting materials are not commercially available, they may be prepared by standard synthetic techniques. In general, the synthesis pathways for any individual compound of Formula I, II, III, IV or V and related formulae will depend on the specific substituents of each molecule, such factors being appreciated by those of ordinary skilled in the art. The following general methods and procedures described hereinafter in the examples may be employed to prepare compounds of Formula I, II, III, IV or V and related formulae. Reaction conditions depicted in the following schemes, such as temperatures, solvents, or co-reagents, are given as examples only and are not restrictive. It will be appreciated that where typical or preferred experimental conditions (i.e. reaction temperatures, time, moles of reagents, solvents etc.) are given, other experimental conditions can also be used unless otherwise stated. Optimum reaction conditions may vary with the particular reactants or solvents used, but such conditions can be determined by the person skilled in the art, using routine optimization procedures. For all the protection and deprotection methods, see Philip J. Kocienski, “Protecting Groups”, Georg Thieme Verlag Stuttgart, New York, 1994 and, Theodora W. Greene and Peter G. M. Wuts, “Protective Groups in Organic Synthesis”, Wiley Interscience, 3rd Edition 1999.

### Example 1: Synthesis of Compound 1 N1-[2-(dimethylamino)ethyl]-2-fluoro-N4-(7-{8-methyl-1H,2H,3H-pyrido[2,3-b][1,4]oxazin-7-yl}-5H,6H,7H,8H-pyrido[3,4-d]pyrimidin-2-yl)benzene-1,4-diamine

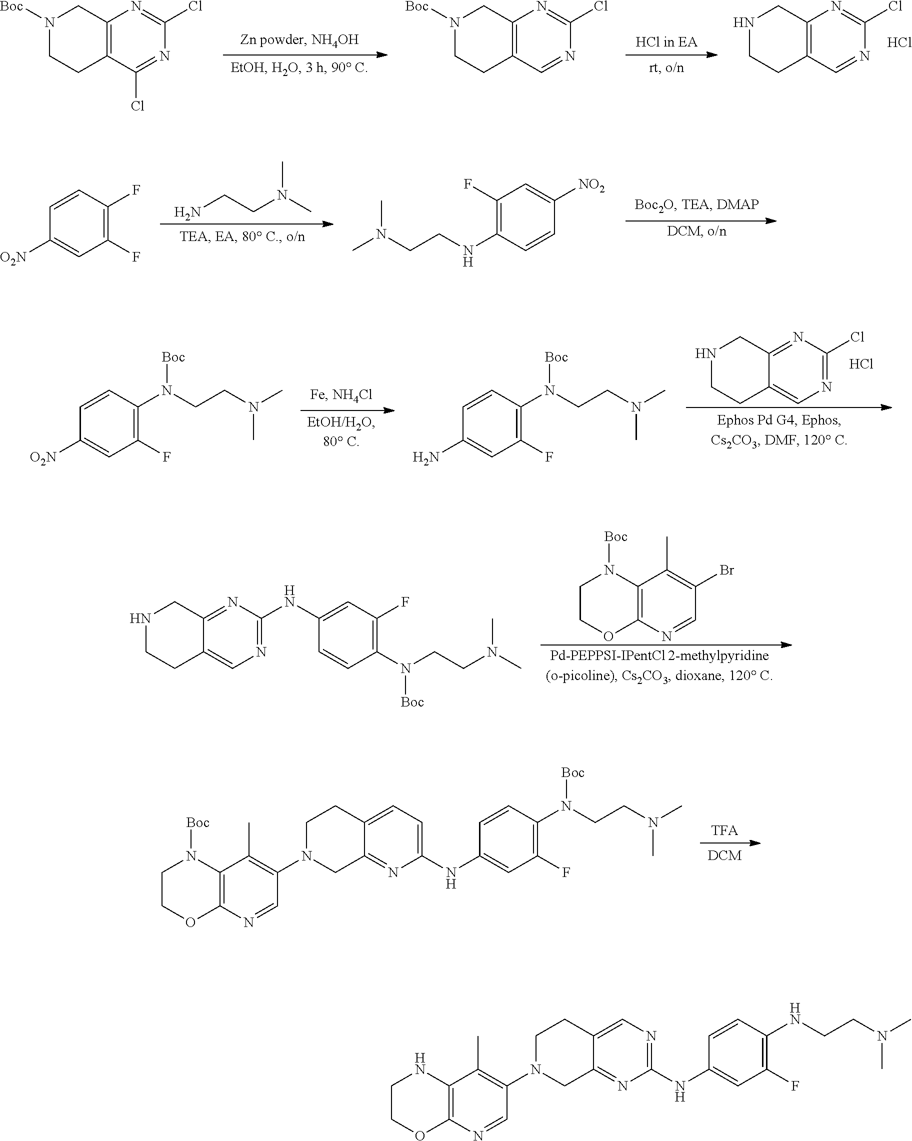

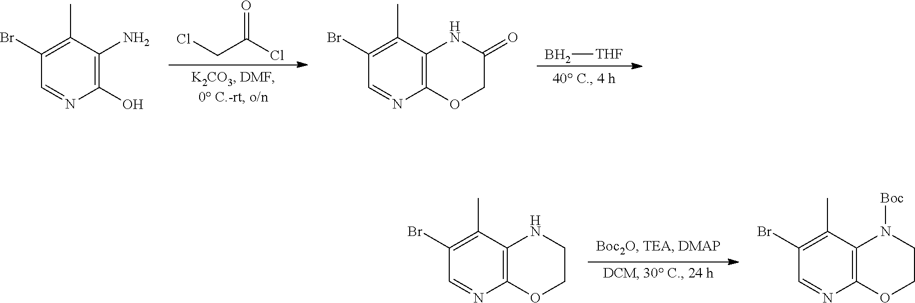

N-[2-(dimethylamino)ethyl]-2-fluoro-4-nitroaniline: A solution of 1,2-difluoro-4-nitrobenzene (3.00 g; 17.91 mmol) and (2-aminoethyl)dimethylamine (1.70 g; 18.32 mmol) and TEA {triethylamine} (5.49 mL; 37.55 mmol) in AcOEt {ethyl acetate} (100.00 mL) was stirred for overnight at 80° C. under nitrogen atmosphere. The resulting mixture was concentrated under vacuum. The residue was purified by silica gel column chromatography and eluted with (PE {petroleum ether}: AcOEt=7:3) to afford N-[2-(dimethylamino)ethyl]-2-fluoro-4-nitroaniline (3.77 g; 90.9%) as yellow oil.

tert-butyl N-[2-(dimethylamino)ethyl]-N-(2-fluoro-4-nitrophenyl)carbamate: A solution of N-[2-(dimethylamino)ethyl]-2-fluoro-4-nitroaniline (3.67 g; 15.85 mmol) and (Boc)₂O (17.25 mL; 76.61 mmol) and DMAP {4-Dimethylaminopyridine} (200.00 mg; 1.47 mmol) and TEA (6.30 mL; 43.06 mmol) in DCM {dichloromethane} (50.00 mL) was stirred for overnight at room temperature under nitrogen atmosphere. The resulting mixture was concentrated under vacuum. The residue was purified by silica gel column chromatography and eluted with (PE:EtOAc=7:3) to afford tert-butyl N-[2-(dimethylamino)ethyl]-N-(2-fluoro-4-nitrophenyl)carbamate (5.00 g; 96.4%) as a yellow oil.

tert-butyl N-(4-amino-2-fluorophenyl)-N-[2-(dimethylamino)ethyl]carbamate: A solution of tert-butyl N-[2-(dimethylamino)ethyl]-N-(2-fluoro-4-nitrophenyl)carbamate (4.90 g; 14.97 mmol) and NH₄Cl (4.00 g; 74.03 mmol) and Fe (4.00 g; 70.19 mmol) in water (32.00 mL) and EtOH (48.00 mL) was stirred for 3 h at 80 degree C. under nitrogen atmosphere. The resulting mixture was concentrated under vacuum. The residue was purified by silica gel column chromatography and eluted with (PE:EtOAc=1:99) to afford tert-butyl N-(4-amino-2-fluorophenyl)-N-[2-(dimethylamino)ethyl]carbamate (2.00 g; 6.50 mmol; 43.4%) as an off-white solid.

tert-butyl 2-chloro-5H,6H,7H,8H-pyrido[3,4-d]pyrimidine-7-carboxylate: To a solution of tert-butyl 2,4-dichloro-5H,6H,7H,8H-pyrido[3,4-d]pyrimidine-7-carboxylate (20.00 g; 62.46 mmol) in EtOH (400.00 mL) was added Zn (22.00 g; 319.67 mmol) and NH₄OH (24.00 mL) at room temperature. The resulting mixture was stirred for 15 h at 90° C. under N₂ atmosphere. The reaction was cooled, filtered through Celite, and washed with ethyl acetate. The filtrate was concentrated under reduced pressure. The residue was purified by silica gel column and eluted with ethyl acetate (EA):PE=1:3 to afford tert-butyl 2-chloro-5H,6H,7H,8H-pyrido[3,4-d]pyrimidine-7-carboxylate (13.00 g; 77.2%) as a light-yellow oil.

2-chloro-5H,6H,7H,8H-pyrido[3,4-d]pyrimidine hydrochloride: To a stirred mixture of tert-butyl 2-chloro-5H,6H,7H,8H-pyrido[3,4-d]pyrimidine-7-carboxylate (1.00 g; 3.71 mmol) in HCl (g) in EA (10.00 mL; 40.00 mmol) and EtOAc (10.00 mL). The resulting mixture was stirred for 2 h at 25° C. under nitrogen atmosphere. The resulting mixture was extracted with EA (3×50 mL). the filtrate was concentrated under reduced pressure to afford 2-chloro-5H,6H,7H,8H-pyrido[3,4-d]pyrimidine hydrochloride (700 mg; 98%) as a red solid.

tert-butyl N-[2-(dimethylamino)ethyl]-N-[2-fluoro-4-({5H,6H,7H,8H-pyrido[3,4-d]pyrimidin-2-yl}amino)phenyl]carbamate: A solution of tert-butyl N-(4-amino-2-fluorophenyl)-N-[2-(dimethylamino)ethyl]carbamate (300.00 mg; 0.98 mmol) and 2-chloro-5H,6H,7H,8H-pyrido[3,4-d]pyrimidine hydrochloride (252.00 mg; 1.20 mmol) and Ephos (46.00 mg; 0.08 mmol) and EPhos Pd G₄ (54.00 mg; 0.06 mmol) and Cs₂CO₃ (980.00 mg; 2.86 mmol) in DMF {N, N-Dimethylformamide} (6.00 mL) was stirred for 2 h at 120° C. under nitrogen atmosphere. The resulting mixture was concentrated under vacuum. The residue was purified by silica gel column chromatography and eluted with (PE:EtOAc=7:3) to afford tert-butyl N-[2-(dimethylamino)ethyl]-N-[2-fluoro-4-({5H,6H,7H,8H-pyrido[3,4-d]pyrimidin-2-yl}amino)phenyl]carbamate (270.00 mg; 50.9%) as a yellow solid.

7-bromo-8-methyl-1H,3H-pyrido[2,3-b][1,4]oxazin-2-one: To a stirred mixture of 3-amino-5-bromo-4-methylpyridin-2-ol (100.00 g, 467.90, 1.0 equiv.) and K₂CO₃ (205.00 g, 1409.19 mmol) in N,N-dimethylmethanamide (DMF) (600.00 mL) was added chloroacetyl chloride (1.22 g, 10.294 mmol) dropwise at 0° C. The resulting mixture was stirred for overnight at room temperature under argon atmosphere. The resulting mixture was poured into ice-cold water (3.00 L) and stirred for 15 min. The solid wad filtered and dried under vacuum to afford 7-bromo-8-methyl-1H,3H-pyrido[2,3-b][1,4]oxazin-2-one (110 g, crude product) as a gray solid.

7-bromo-8-methyl-2,3-dihydro-1H-pyrido[2,3-b][1,4]oxazine: A mixture of 7-bromo-8-methyl-1H,3H-pyrido[2,3-b][1,4]oxazin-2-one (110.00 g, 424.19 mmol) and BH₃-tetrahydrofuran (THF) (550.00 mL, 550.00 mmol, 1 Mol/L) was stirred for 4 h at 40° C. under nitrogen atmosphere. The resulting mixture was quenched with MeOH (˜550 mL) carefully at 0° C. To the mixture was added HCl (2M, 550 mL) with stirring. The solution was stirred for about 30 min at room temperature. The resulting mixture was concentrated to half of the volume and adjusted pH=7-8 by Na₂CO₃ (aq.). The final solution was extracted with EtOAc. The combined organic layers were washed with brine and dried over anhydrous Na₂SO₄. After filtration, the filtrate was concentrated under reduced pressure. This resulted in 7-bromo-8-methyl-1H,2H,3H-pyrido[2,3-b][1,4]oxazine (65.00 g, crude product) as a brown solid.

tert-butyl 7-bromo-8-methyl-2H,3H-pyrido[2,3-b][1,4]oxazine-1-carboxylate: To a stirred solution of 7-bromo-8-methyl-1H,2H,3H-pyrido[2,3-b][1,4]oxazine (59.00 g, 239.53 mmol), TEA (107.83 mL, 736.94 mmol) and DMAP (6.40 g, 49.77 mmol) in DCM (350 mL) was added Boc₂O (169.30 g, 736.94 mmol) in DCM (150.00 mL) dropwise at 0° C. The resulting mixture was stirred for overnight at 30° C. under nitrogen atmosphere (˜70% conversion in the LCMS), then TEA (53.915 mL, 368.47 mmol) and Boc₂O (84.65 g, 368.47 mmol) was added at room temperature. The resulting mixture was stirred for additional 7 h at 30° C. under nitrogen atmosphere. The resulting mixture was added water. The resulting mixture was extracted with DCM. The combined organic layers were dried over anhydrous Na₂SO₄. After filtration, the filtrate was concentrated under reduced pressure. The residue was purified by silica gel column chromatography and eluted with 15% EtOAc in PE to afford tert-butyl 7-bromo-8-methyl-2H,3H-pyrido[2,3-b][1,4]oxazine-1-carboxylate (67.00 g, 79.7%) as a yellow solid.

tert-butyl 7-{2-[(4-{[(tert-butoxy)carbonyl][2-(dimethylamino)ethyl]amino}-3-fluorophenyl)amino]-5H,6H,7H,8H-pyrido[3,4-d]pyrimidin-7-yl}-8-methyl-1H,2H,3H-pyrido[2,3-b][1,4]oxazine-1-carboxylate: A solution of tert-butyl N-[2-(dimethylamino)ethyl]-N-[2-fluoro-4-({5H,6H,7H,8H-pyrido[3,4-d]pyrimidin-2-yl}amino)phenyl]carbamate (200.00 mg; 0.37 mmol) and tert-butyl 7-bromo-8-methyl-1H,2H,3H-pyrido[2,3-b][1,4]oxazine-1-carboxylate (305.00 mg; 0.88 mmol) and Pd-PEPPSI-IPentCl 2-methylpyridine (o-picoline) (40.00 mg; 0.05 mmol) and Cs₂CO₃ (450.00 mg; 1.31 mmol) in 1,4-dioxane (5.00 mL) was stirred for 4 h at 120° C. under nitrogen atmosphere. The resulting mixture was concentrated under vacuum. The residue was purified by silica gel column chromatography and eluted with (PE:EtOAc=7:3) to afford tert-butyl 7-{2-[(4-{[(tert-butoxy)carbonyl][2-(dimethylamino)ethyl]amino}-3-fluorophenyl)amino]-5H,6H,7H,8H-pyrido[3,4-d]pyrimidin-7-yl}-8-methyl-1H,2H,3H-pyrido[2,3-b][1,4]oxazine-1-carboxylate (130.00 mg; 44.3%) as a yellow solid.

N1-[2-(dimethylamino)ethyl]-2-fluoro-N4-(7-{8-methyl-1H,2H,3H-pyrido[2,3-b][1,4]oxazin-7-yl}-5H,6H,7H,8H-pyrido[3,4-d]pyrimidin-2-yl)benzene-1,4-diamine: A solution of tert-butyl 7-{2-[(4-{[(tert-butoxy) carbonyl][2-(dimethylamino)ethyl]amino}-3-fluorophenyl)amino]-5H,6H,7H,8H-pyrido[3,4-d]pyrimidin-7-yl}-8-methyl-1H,2H,3H-pyrido[2,3-b][1,4]oxazine-1-carboxylate (130.00 mg; 0.16 mmol) and TFA (2.00 mL) in DCM (6.0 mL) was stirred for 1 h at room temperature under nitrogen atmosphere. The resulting mixture was concentrated under vacuum. The residue was purified by Column: X Bridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 25% B to 55% B in 9 min., 55% B; Wave Length: 254 nm; RT1 (min.): 7; Number Of Runs: 4 to afford N1-[2-(dimethylamino)ethyl]-2-fluoro-N4-(7-{8-methyl-1H,2H,3H-pyrido[2,3-b][1,4]oxazin-7-yl}-5H,6H,7H,8H-pyrido[3,4-d]pyrimidin-2-yl)benzene-1,4-diamine (18.10 mg; 22.1%) as an off-white solid.

HPLC: 95.03% purity, RT=2.21 min. MS: m/z=479.25 [M+H]⁺. ¹H NMR (400 MHz, DMSO-d6) δ 9.30 (s, 1H), 8.28 (s, 1H), 7.60 (dd, J=14.5, 2.4 Hz, 1H), 7.27 (s, 1H), 7.26 (s, 1H), 6.69 (t, J=9.4 Hz, 1H), 5.54 (s, 1H), 4.78 (s, 1H), 4.19 (s, 2H), 3.92 (s, 2H), 3.17-3.08 (m, 5H), 2.76 (s, 1H), 2.55 (s, 4H), 2.29 (s, 6H), 2.03 (s, 3H).

### Example 2: Synthesis of Compound 2 2-(azetidin-1-yl)-N-{4-[(6-{8-chloro-1H,2H,3H-pyrido[2,3-b][1,4]oxazin-7-yl}-5,6,7,8-tetrahydro-2,6-naphthyridin-3-yl)amino]-2-methylphenyl}acetamide

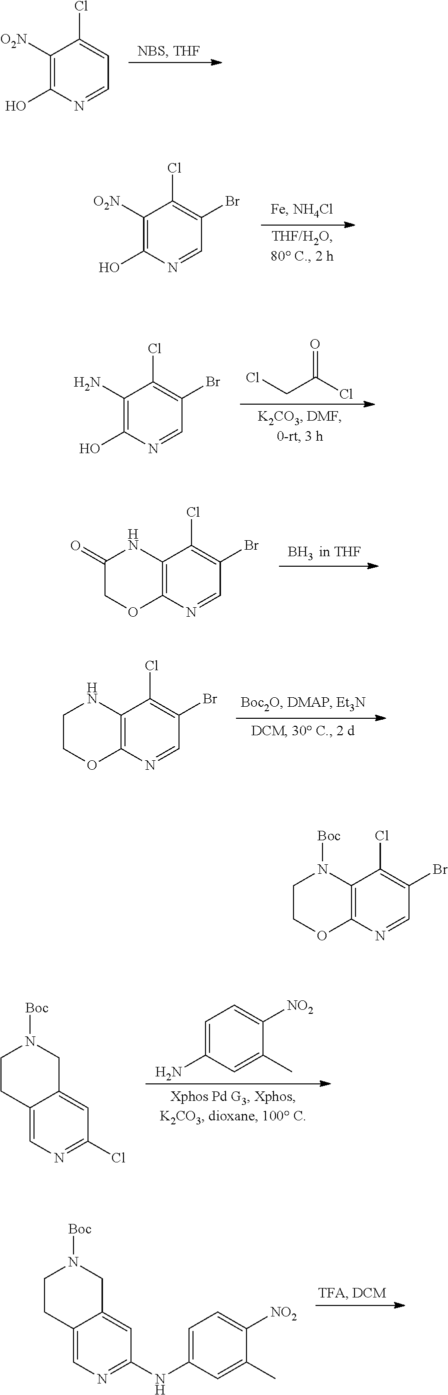

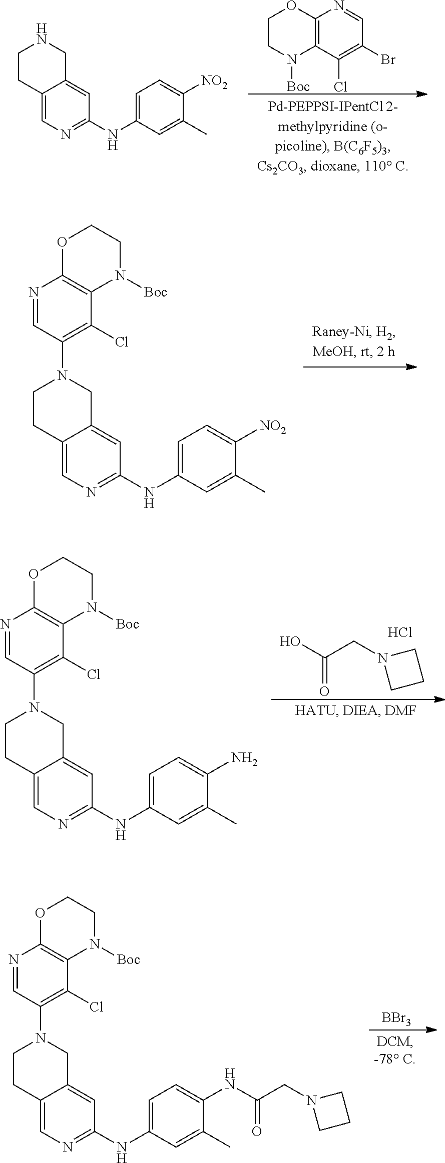

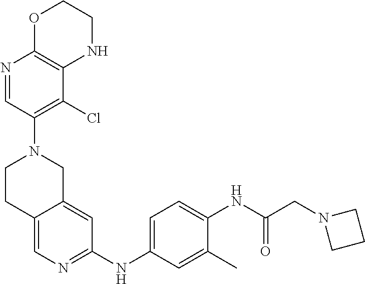 [SMILES: Cc1cc(Nc2cc3c(cn2)CCN(c2cnc4c(c2Cl)NCCO4)C3)ccc1NC(=O)CN1CCC1]

tert-butyl 7-[(3-methyl-4-nitrophenyl)amino]-1,2,3,4-tetrahydro-2,6-naphthyridine-2-carboxylate: To a stirred solution of tert-butyl 7-chloro-1,2,3,4-tetrahydro-2,6-naphthyridine-2-carboxylate (400.00 mg; 1.46 mmol) and 3-methyl-4-nitroaniline (271.84 mg; 1.75 mmol) in 1,4-dioxane (5.00 mL) was added Xphos (146.44 mg; 0.29 mmol), Xphos Pd G₃ (130.01 mg; 0.15 mmol) and K₂CO₃ (423.63 mg; 2.92 mmol) in portion at room temperature. The resulting mixture was stirred for 2 h at 100° C. under nitrogen atmosphere. The resulting mixture was concentrated under reduced pressure. The residue was purified by silica gel column chromatography and eluted with PE/EA (6:1) to afford tert-butyl 7-[(3-methyl-4-nitrophenyl)amino]-1,2,3,4-tetrahydro-2,6-naphthyridine-2-carboxylate (330.00 mg; 58.74%) as a yellow solid.

N-(3-methyl-4-nitrophenyl)-5,6,7,8-tetrahydro-2,6-naphthyridin-3-amine: To a stirred solution of tert-butyl 7-[(3-methyl-4-nitrophenyl)amino]-1,2,3,4-tetrahydro-2,6-naphthyridine-2-carboxylate (330.00 mg; 0.86 mmol; 1.00 eq.) in DCM (15.00 mL) was added TFA (3.00 mL) in portion at room temperature. After stirring for 1 h at room temperature, the mixture was basified to pH [7] with saturated NaHCO₃ (aq.). The resulting mixture was extracted with EtOAc. The combined organic layers were washed with brine and dried over anhydrous Na₂SO₄. After filtration, the filtrate was concentrated under reduced pressure to afford N-(3-methyl-4-nitrophenyl)-5,6,7,8-tetrahydro-2,6-naphthyridin-3-amine (220.00 mg; crude product) as a yellow solid.

5-bromo-4-chloro-3-nitropyridin-2-ol: To a stirred solution of 4-chloro-3-nitropyridin-2-ol (75.00 g; 408.21 mmol) in THF (700.00 mL) was added N-bromosuccinimide (NBS) (89.88 g; 489.85 mmol) in portions at room temperature. The resulting mixture was stirred for overnight at room temperature. The resulting mixture was diluted with water (200 mL), extracted with EtOAc (3×100 mL). The combined organic layers were washed with brine (150 mL) and dried over anhydrous Na₂SO₄. The resulting mixture was concentrated under reduced pressure. The residue was purified by slurry with PE:EA=3:1. The solid was collected to afford 5-bromo-4-chloro-3-nitropyridin-2-ol (110.00 g; crude product) as a yellow solid.

3-amino-5-bromo-4-chloropyridin-2-ol: To a stirred solution of 5-bromo-4-chloro-3-nitropyridin-2-ol (75.00 g; 270.15 mmol) and NH₄Cl (60.84 g; 1080.62 mmol) in water (500.00 mL) and THF (900.00 mL) was added Fe (79.62 g; 1350.77 mmol) and in portions at room temperature. The resulting mixture was stirred for 1 h at 70° C. The resulting mixture was filtered and the filter cake was washed with THF (1000 mL). The resulting mixture was extracted with EtOAc (100 mL*3). The combined organic layers were washed with brine (300 mL) and dried over anhydrous Na₂SO₄. After filtration, the filtrate was concentrated under reduced pressure to afford 3-amino-5-bromo-4-chloropyridin-2-ol (70.00 g; Crude Product) as brown solid.

7-bromo-8-chloro-1H,2H,3H-pyrido[2,3-b][1,4]oxazin-2-one: To a stirred solution of 3-amino-5-bromo-4-chloropyridin-2-ol (60.00 g; 228.34 mmol) and K₂CO₃ (99.44 g; 685.02 mmol) in DMF (720.00 mL) was added 2-chloroacetyl chloride (47.79 mL; 570.85 mmol) in portions at 0° C. under argon atmosphere. The resulting mixture was stirred for 6 h at room temperature and for 1 h at 40° C. The resulting mixture was diluted with water (3000 mL). The resulting mixture was filtered, and the filter cake was washed with water (1000 mL). The filter cake was dried under reduced pressure to afford 7-bromo-8-chloro-1H,2H,3H-pyrido[2,3-b][1,4]oxazin-2-one (40.00 g; crude product) as a brown solid.

7-bromo-8-chloro-1H,2H,3H-pyrido[2,3-b][1,4]oxazine: A solution of 7-bromo-8-chloro-1H,2H,3H-pyrido[2,3-b][1,4]oxazin-2-one (35.00 g; 130.59 mmol) in BH₃ in THF (350.00 mL; 350.00 mmol) was stirred for 2 h at 40° C. under N₂ atmosphere. The resulting mixture was quenched with water (200 mL) and extracted with EtOAc (100 mL*3). The combined organic layers were washed with brine (150 mL). The residue was purified by silica gel column chromatography and eluted with PE/EtOAc (3:1) to afford 7-bromo-8-chloro-1H,2H,3H-pyrido[2,3-b][1,4]oxazine (26.00 g; 71.34%) as a white solid.

tert-butyl 7-bromo-8-chloro-1H,2H,3H-pyrido[2,3-b][1,4]oxazine-1-carboxylate: To a stirred solution of 7-bromo-8-chloro-1H,2H,3H-pyrido[2,3-b][1,4]oxazine (340.00 mg; 1.18 mmol) and Di-tert-butyl dicarbonate (897.00 mg; 3.90 mmol) in Dichloromethane (5.00 mL) were added Triethylamine (0.57 mL; 3.91 mmol) and N,N-dimethylpyridin-4-amine (34.00 mg; 0.26 mmol) in portions at room temperature under N₂ atmosphere. The resulting mixture was stirred for 4 h at 30° C. under N₂ atmosphere. The resulting mixture was diluted with water (50 mL) and extracted with DCM (3×50 mL). The combined organic layers were washed with brine (50 mL) and dried over anhydrous Na₂SO₄. The resulting mixture was concentrated under reduced pressure. The residue was purified by silica gel column and eluted with 15% EtOAc in PE to afford tert-butyl 7-bromo-8-chloro-1H,2H,3H-pyrido[2,3-b][1,4]oxazine-1-carboxylate (340.00 mg; 68.6%) as a white solid.

tert-butyl 8-chloro-7-{7-[(3-methyl-4-nitrophenyl)amino]-1,2,3,4-tetrahydro-2,6-naphthyridin-2-yl}-1H,2H,3H-pyrido[2,3-b][1,4]oxazine-1-carboxylate: To a stirred solution of N-(3-methyl-4-nitrophenyl)-5,6,7,8-tetrahydro-2,6-naphthyridin-3-amine (520.00 mg; 1.74 mmol) and tert-butyl 7-bromo-8-chloro-1H,2H,3H-pyrido[2,3-b][1,4]oxazine-1-carboxylate (765.73 mg; 2.08 mmol) in 1,4-dioxane (18.00 mL) was added Pd-PEPPSI-IPentCl 2-methylpyridine (o-picoline) (153.49 mg; 0.17 mmol) and Cs₂CO₃ (1190.50 mg; 3.47 mmol) in portion at room temperature. The resulting mixture was stirred for 16 h at 110° C. under nitrogen atmosphere. The resulting mixture was concentrated under reduced pressure. The residue was purified by silica gel column chromatography and eluted with PE/EA (1:1) to afford tert-butyl 8-chloro-7-{7-[(3-methyl-4-nitrophenyl)amino]-1,2,3,4-tetrahydro-2,6-naphthyridin-2-yl}-1H,2H,3H-pyrido[2,3-b][1,4]oxazine-1-carboxylate (120.00 mg; 12.14%) as a yellow solid.

tert-butyl 7-{7-[(4-amino-3-methylphenyl)amino]-1,2,3,4-tetrahydro-2,6-naphthyridin-2-yl}-8-chloro-1H,2H,3H-pyrido[2,3-b][1,4]oxazine-1-carboxylate: To a solution of tert-butyl 8-chloro-7-{7-[(3-methyl-4-nitrophenyl)amino]-1,2,3,4-tetrahydro-2,6-naphthyridin-2-yl}-1H,2H,3H-pyrido[2,3-b][1,4]oxazine-1-carboxylate (110.00 mg; 0.19 mmol) in MeOH (10.00 mL) was added Raney-Ni (20.04 mg; 0.22 mmol) at room temperature. After stirring for 1 h at room temperature under hydrogen atmosphere, the resulting mixture was filtered. The filter cake was washed with MeOH and then filtrate was concentrated under reduced pressure to afford tert-butyl 7-{7-[(4-amino-3-methylphenyl)amino]-1,2,3,4-tetrahydro-2,6-naphthyridin-2-yl}-8-chloro-1H,2H,3H-pyrido[2,3-b][1,4]oxazine-1-carboxylate (40.00 mg; crude product) as a white solid.

tert-butyl 7-[7-({4-[2-(azetidin-1-yl)acetamido]-3-methylphenyl}amino)-1,2,3,4-tetrahydro-2,6-naphthyridin-2-yl]-8-chloro-1H,2H,3H-pyrido[2,3-b][1,4]oxazine-1-carboxylate: To a stirred solution of tert-butyl 7-{7-[(4-amino-3-methylphenyl)amino]-1,2,3,4-tetrahydro-2,6-naphthyridin-2-yl}-8-chloro-1H,2H,3H-pyrido[2,3-b][1,4]oxazine-1-carboxylate (65.00 mg; 0.12 mmol) and 2-(azetidin-1-yl)acetic acid hydrochloride (36.73 mg; 0.23 mmol) in DMF (5.00 mL) was added HATU (93.11 mg; 0.23 mmol) and DIEA (47.46 mg; 0.35 mmol) in portion at room temperature. The resulting mixture was stirred for 6 h at 40° C. under nitrogen atmosphere. The resulting mixture was concentrated under reduced pressure. The residue was purified by silica gel column chromatography and eluted with CH₂Cl₂/MeOH (10:1) to afford tert-butyl 7-[7-({4-[2-(azetidin-1-yl)acetamido]-3-methylphenyl}amino)-1,2,3,4-tetrahydro-2,6-naphthyridin-2-yl]-8-chloro-1H,2H,3H-pyrido[2,3-b][1,4]oxazine-1-carboxylate (50.00 mg; 59.40%) as a yellow solid.

2-(azetidin-1-yl)-N-{4-[(6-{8-chloro-1H,2H,3H-pyrido[2,3-b][1,4]oxazin-7-yl}-5,6,7,8-tetrahydro-2,6-naphthyridin-3-yl)amino]-2-methylphenyl}acetamide: To a stirred mixture of tert-butyl 7-[7-({4-[2-(azetidin-1-yl)acetamido]-3-methylphenyl}amino)-1,2,3,4-tetrahydro-2,6-naphthyridin-2-yl]-8-chloro-1H,2H,3H-pyrido[2,3-b][1,4]oxazine-1-carboxylate (50.00 mg; 0.07 mmol) in DCM (4.00 mL) was added BBr₃ in DCM (0.40 mL; 0.40 mmol) in portion at −78° C. The resulting mixture was stirred for 1 h at −78° C. under nitrogen atmosphere. The resulting mixture was concentrated under reduced pressure. The crude product was purified by Prep-HPLC with the following conditions Column: X Bridge Shield RP18 OBD Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 55% B to 85% B in 9 min., 85% B; Wave Length: 254 nm; RTT (min.): 7; Number Of Runs: 2 to afford 2-(azetidin-1-yl)-N-{4-[(6-{8-chloro-1H,2H,3H-pyrido[2,3-b][1,4]oxazin-7-yl}-5,6,7,8-tetrahydro-2,6-naphthyridin-3-yl)amino]-2-methylphenyl}acetamide (7.30 mg; 020.23%) as a white solid.

HPLC: 99.59% purity, RT=2.14 min. MS: m/z=520.15 [M+H]⁺. ¹H NMR (300 MHz, DMSO-d6): δ 9.03 (s, 1H), 8.74 (s, 1H), 7.99 (s, 1H), 7.40 (d, J=9.5 Hz, 2H), 7.31 (d, J=17.1 Hz, 2H), 6.58 (s, 1H), 6.10 (s, 1H), 4.24 (s, 2H), 4.10 (s, 2H), 3.30 (s, 4H), 3.19 (d, J=16.2 Hz, 4H), 3.10 (s, 2H), 2.83 (s, 2H), 2.16 (s, 3H), 2.06 (q, J=6.9 Hz, 2H).

### Example 3: Synthesis of Compound 3 2-(azetidin-1-yl)-N-{2-methyl-4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}acetamide

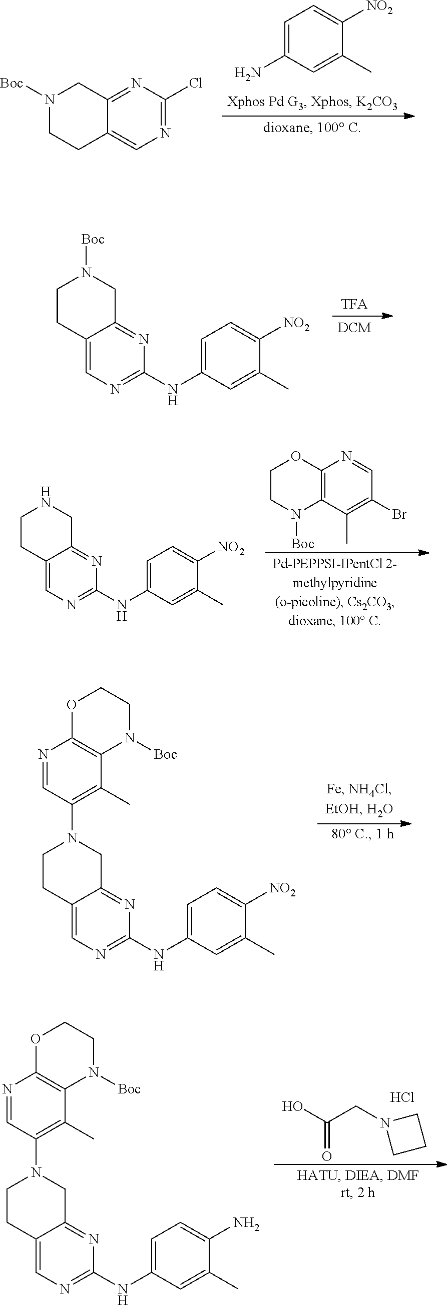

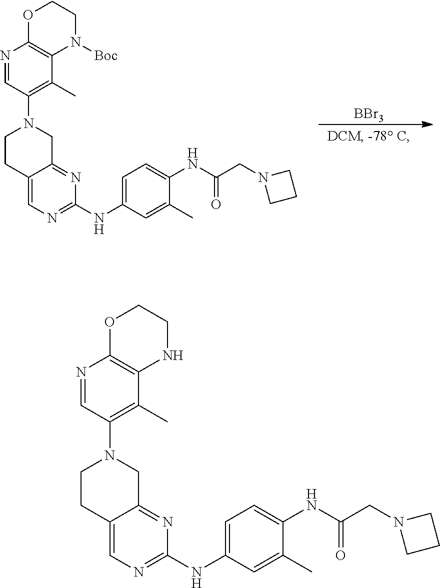 [SMILES: Cc1cc(Nc2ncc3c(n2)CN(c2cnc4c(c2C)N(C(=O)OC(C)(C)C)CCO4)CC3)ccc1NC(=O)CN1CCC1 ; "BBr3" ; "DCM, -78° C," ; Cc1cc(Nc2ncc3c(n2)CN(c2cnc4c(c2C)NCCO4)CC3)ccc1NC(=O)CN1CCC1]

tert-butyl 2-[(3-methyl-4-nitrophenyl)amino]-5H,6H,7H,8H-pyrido[3,4-d]pyrimidine-7-carboxylate: To a stirred mixture of tert-butyl 2-chloro-5H,6H,7H,8H-pyrido[3,4-d]pyrimidine-7-carboxylate (800.00 mg; 2.89 mmol) and 3-methyl-4-nitroaniline (590.00 mg; 3.76 mmol) in 1,4-dioxane (15.00 mL) were added X-PHOS (290.00 mg; 0.58 mmol), K₂CO₃ (1261.00 mg; 8.67 mmol) and XPhos Pd G₃ (257.00 mg; 0.29 mmol) in portions at 25° C. The resulting mixture was stirred for 2 h at 100° C. under nitrogen atmosphere. The residue was purified by silica gel column chromatography and eluted with PE:EA=70:30 to afford tert-butyl 2-[(3-methyl-4-nitrophenyl)amino]-5H,6H,7H,8H-pyrido[3,4-d]pyrimidine-7-carboxylate (1.00 g; 89.8%) as a yellow solid.

N-(3-methyl-4-nitrophenyl)-5H,6H,7H,8H-pyrido[3,4-d]pyrimidin-2-amine: To a stirred mixture of tert-butyl 2-[(3-methyl-4-nitrophenyl)amino]-5H,6H,7H,8H-pyrido[3,4-d]pyrimidine-7-carboxylate (980.00 mg; 2.54 mmol) in DCM (20.00 mL) was added TFA (4.00 mL) in portions at 25° C. The resulting mixture was stirred for 2 h at 25° C. under nitrogen atmosphere. The resulting mixture was concentrated under vacuum. The reaction was diluted with NaHCO₃ (aq.) at 25° C. The resulting mixture was extracted with EA (3×80 mL). The combined organic layers were washed with brine and dried over anhydrous MgSO₄. After filtration, the filtrate was concentrated under reduced pressure to afford N-(3-methyl-4-nitrophenyl)-5H,6H,7H,8H-pyrido[3,4-d]pyrimidin-2-amine (700.00 mg; crude product) as yellow solid.

tert-butyl 8-methyl-7-{2-[(3-methyl-4-nitrophenyl)amino]-5H,6H,7H,8H-pyrido[3,4-d]pyrimidin-7-yl}-1H,2H,3H-pyrido[2,3-b][1,4]oxazine-1-carboxylate: To a stirred mixture of N-(3-methyl-4-nitrophenyl)-5H,6H,7H,8H-pyrido[3,4-d]pyrimidin-2-amine (700.00 mg; 2.45 mmol) and tert-butyl 7-bromo-8-methyl-1H,2H,3H-pyrido[2,3-b][1,4]oxazine-1-carboxylate (600.00 mg; 1.64 mmol) in 1,4-dioxane (15.00 mL) were added Cs₂CO₃ (1.90 g; 5.54 mmol) and Pd-PEPPSI-IPentCl 2-methylpyridine (o-picoline) (130.00 mg; 0.15 mmol) in portions at 25° C. The resulting mixture was stirred for 12 h at 100° C. under nitrogen atmosphere. The resulting mixture was concentrated under vacuum. The residue was purified by silica gel column chromatography and eluted with PE:EA=1:1 to afford tert-butyl 8-methyl-7-{2-[(3-methyl-4-nitrophenyl)amino]-5H,6H,7H,8H-pyrido[3,4-d]pyrimidin-7-yl}-1H,2H,3H-pyrido[2,3-b][1,4]oxazine-1-carboxylate (700.00 mg; 80.0%) as a yellow solid.

7-{2-[(4-amino-3-methylphenyl)amino]-5H,6H,7H,8H-pyrido[3,4-d]pyrimidin-7-yl}-8-methyl-1H,2H,3H-pyrido[2,3-b][1,4]oxazine-1-carboxylate: To a stirred mixture of tert-butyl 8-methyl-7-{2-[(3-methyl-4-nitrophenyl)amino]-5H,6H,7H,8H-pyrido[3,4-d]pyrimidin-7-yl}-1H,2H,3H-pyrido[2,3-b][1,4]oxazine-1-carboxylate (680.00 mg; 1.27 mmol) and Fe (225.00 mg; 3.83 mmol) in EtOH (9.00 mL) were added NH₄Cl (276.00 mg; 5.11 mmol) in H₂O (6.00 mL) in portions at 25° C. The resulting mixture was stirred for 2 h at 80° C. under nitrogen atmosphere. The residue was purified by silica gel column chromatography and eluted with EA to afford tert-butyl 7-{2-[(4-amino-3-methylphenyl)amino]-5H,6H,7H,8H-pyrido[3,4-d]pyrimidin-7-yl}-8-methyl-1H,2H,3H-pyrido[2,3-b][1,4]oxazine-1-carboxylate (600.00 mg, 93.5%) as a yellow solid.

tert-butyl 7-[2-({4-[2-(azetidin-1-yl)acetamido]-3-methylphenyl}amino)-5H,6H,7H,8H-pyrido[3,4-d]pyrimidin-7-yl]-8-methyl-1H,2H,3H-pyrido[2,3-b][1,4]oxazine-1-carboxylate: To a stirred mixture of tert-butyl 7-{2-[(4-amino-3-methylphenyl)amino]-5H,6H,7H,8H-pyrido[3,4-d]pyrimidin-7-yl}-8-methyl-1H,2H,3H-pyrido[2,3-b][1,4]oxazine-1-carboxylate (180.00 mg; 0.29 mmol) and 2-(azetidin-1-yl)acetic acid hydrochloride (82.21 mg; 0.58 mmol) in DMF (5.00 mL) were added HATU (349.35 mg; 0.87 mmol) and DIEA (79.14 mg; 0.58 mmol) at room temperature. The mixture was stirred for 1 h at room temperature. The resulting mixture was quenched by ice/water and extracted with EtOAc. The combined organic layers were washed with brine and dried over anhydrous Na₂SO₄. After filtration, the filtrate was concentrated under reduced pressure. The resulting mixture was concentrated under reduced pressure and purified by silica gel column chromatography and eluted with CH₂Cl₂/MeOH (10:1) to afford tert-butyl 7-[2-({4-[2-(azetidin-1-yl)acetamido]-3-methylphenyl}amino)-5H,6H,7H,8H-pyrido[3,4-d]pyrimidin-7-yl]-8-methyl-1H,2H,3H-pyrido[2,3-b][1,4]oxazine-1-carboxylate (130.00 mg; 67.8%) as a yellow solid.

2-(azetidin-1-yl)-N-{2-methyl-4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}acetamide: To a stirred mixture of tert-butyl 7-[2-({4-[2-(azetidin-1-yl)acetamido]-3-methylphenyl}amino)-5H,6H,7H,8H-pyrido[3,4-d]pyrimidin-7-yl]-8-methyl-1H,2H,3H-pyrido[2,3-b][1,4]oxazine-1-carboxylate (190.00 mg; 0.28 mmol) in DCM (5.00 mL) was added BBr₃ in DCM (1.00 mL; 1.00 mmol) dropwise at −78° C. The resulting mixture was quenched by NaHCO₃ (aq.) at −20° C. The mixture was extracted with DCM and concentrated under reduced pressure. The residue was purified by Prep-HPLC (Column: Xselect CSH C18 OBD Column 30*150 mm, 5 μm, n; Mobile Phase A: water (0.1% FA), Mobile Phase B: ACN; Flow rate: 60 mL/min.; Gradient: 5% B to 27% B in 8 min., 27% B; Wave Length: 254/220 nm; RT1 (min.): 6.7) to give 2-(azetidin-1-yl)-N-{2-methyl-4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}acetamide (17.10 mg; 11.9%) as a white solid.

HPLC: 97.34% purity, RT=2.29 min. MS: m/z=501.30 [M+H]⁺. ¹H NMR (300 MHz, DMSO-d6): δ 9.40 (s, 1H), 9.05 (s, 1H), 8.30 (s, 1H), 7.58-7.49 (m, 2H), 7.34 (d, J=9.3 Hz, 1H), 7.27 (s, 1H), 5.51 (s, 1H), 4.17 (t, J=4.3 Hz, 2H), 3.93 (s, 2H), 3.30 (s, 6H), 3.13 (d, J=14.3 Hz, 4H), 2.77 (t, J=5.3 Hz, 2H), 2.14 (s, 3H), 2.11-1.95 (m, 1H), 2.02 (s, 4H).

### Example 4: Synthesis of Compound 4 2-fluoro-N4-(6-{8-methyl-1H,2H,3H-pyrido[2,3-b][1,4]oxazin-7-yl}-5,6,7,8-tetrahydro-2,6-naphthyridin-3-yl)-N1-[2-(morpholin-4-yl)ethyl]benzene-1,4-diamine

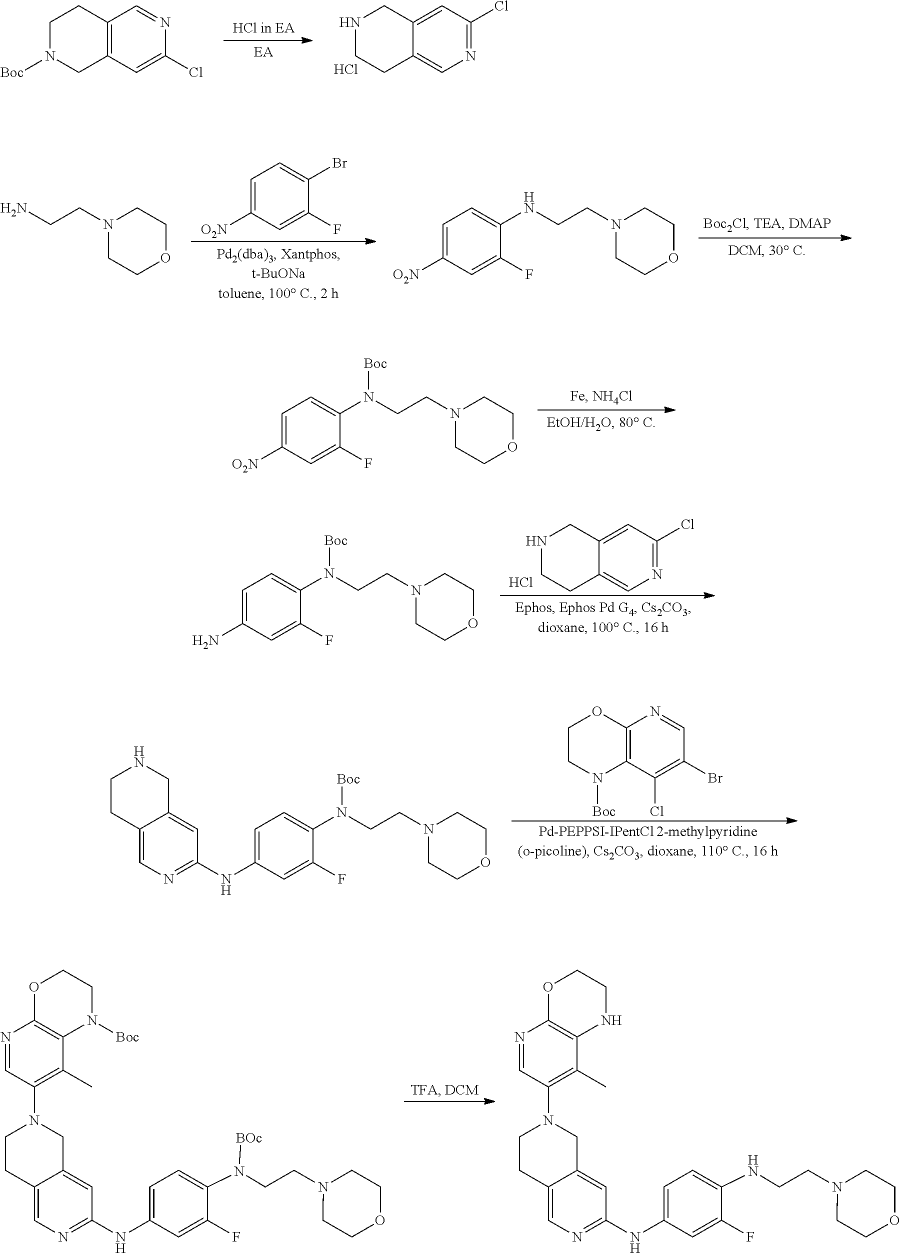

2-fluoro-N-[2-(morpholin-4-yl)ethyl]-4-nitroaniline: To a stirred solution of 1-bromo-2-fluoro-4-nitrobenzene (6.40 g; 29.09 mmol; 1.00 eq.) and 2-(morpholin-4-yl)ethan-1-amine (3.80 g; 29.19 mmol) in Toluene (100.00 mL) were added Pd₂(dba)₃ (2.70 g; 2.65 mmol), Xantphos (3.40 g; 5.82 mmol) and t-BuONa (8.40 g; 78.67 mmol) in portions at room temperature under N₂ atmosphere. The resulting mixture was stirred for 2 h at 100° C. under N₂ atmosphere. The resulting mixture was diluted with water (500 mL), extracted with EtOAc (3×500 mL). The combined organic layers were washed with brine (500 mL) and dried over anhydrous Na₂SO₄. The resulting mixture was concentrated under reduced pressure. The residue was purified by silica gel column and eluted with 80% EtOAc in PE to afford 2-fluoro-N-[2-(morpholin-4-yl)ethyl]-4-nitroaniline (6.40 g; 78.3%) as a yellow solid.

tert-butyl N-(2-fluoro-4-nitrophenyl)-N-[2-(morpholin-4-yl)ethyl]carbamate: To a stirred solution of 2-fluoro-N-[2-(morpholin-4-yl)ethyl]-4-nitroaniline (6.20 g; 22.23 mmol) and Di-tert-butyl dicarbonate (15.00 g; 65.29 mmol) in dichloromethane (100.00 mL) were added Triethylamine (9.62 mL; 65.72 mmol) and N,N-dimethylpyridin-4-amine (563.00 mg; 4.38 mmol) in portions at room temperature under N₂ atmosphere. The resulting mixture was stirred for 4 h at 30° C. under N₂ atmosphere. The resulting mixture was diluted with water (100 mL), extracted with EtOAc (3×100 mL). The combined organic layers were washed with brine (100 mL) and dried over anhydrous Na₂SO₄. The resulting mixture was concentrated under reduced pressure. The residue was purified by silica gel column and eluted with 30% EtOAc in PE to afford tert-butyl N-(2-fluoro-4-nitrophenyl)-N-[2-(morpholin-4-yl)ethyl]carbamate (7.20 g; 87.7%) as a yellow solid.

tert-butyl N-(4-amino-2-fluorophenyl)-N-[2-(morpholin-4-yl)ethyl]carbamate: To a stirred solution of tert-butyl N-(2-fluoro-4-nitrophenyl)-N-[2-(morpholin-4-yl)ethyl]carbamate (7.00 g; 18.95 mmol) in EtOH (3.00 mL) and water (2.00 mL) were added NH₄Cl (4.00 g; 74.03 mmol) and Fe (5.30 g; 89.91 mmol) in portions at room temperature under N₂ atmosphere. The solid was filtered out. The filtrate was diluted with water (500 mL), extracted with EtOAc (3×200 mL). The combined organic layers were washed with brine (200 mL) and dried over anhydrous Na₂SO₄. The resulting mixture was concentrated under reduced pressure to afford tert-butyl N-(4-amino-2-fluorophenyl)-N-[2-(morpholin-4-yl)ethyl]carbamate (6.50 g; crude product) as a yellow solid.

7-chloro-1,2,3,4-tetrahydro-2,6-naphthyridine hydrochloride: To a stirred solution of tert-butyl 7-chloro-1,2,3,4-tetrahydro-2,6-naphthyridine-2-carboxylate (800.00 mg; 2.83 mmol) in 4N HCl in EA (16.00 mL; 64.00 mmol) at room temperature. The resulting mixture was stirred for 1 h at room temperature. The resulting mixture was concentrated under reduced pressure to afford 7-chloro-1,2,3,4-tetrahydro-2,6-naphthyridine hydrochloride (560.00 mg; crude product) as a white solid.

tert-butyl N-{2-fluoro-4-[(5,6,7,8-tetrahydro-2,6-naphthyridin-3-yl)amino]phenyl}-N-[2-(morpholin-4-yl)ethyl]carbamate: To a stirred solution of tert-butyl N-(4-amino-2-fluorophenyl)-N-[2-(morpholin-4-yl)ethyl]carbamate (913.00 mg; 2.69 mmol) and 7-chloro-1,2,3,4-tetrahydro-2,6-naphthyridine hydrochloride (460.00 mg; 2.24 mmol) in 1,4-dioxane (20.00 mL) were added Ephos Pd G₄ (210.00 mg; 0.22 mmol), Ephos (126.00 mg; 0.22 mmol) and Cs₂CO₃ (1477.00 mg; 4.49 mmol) at room temperature under N₂ atmosphere. The resulting mixture was stirred for 16 h at 100° C. under N₂ atmosphere. The reaction was quenched with water at room temperature. The resulting mixture was extracted with EtOAc (3×50 mL). The combined organic layers were washed with brine (1×50 mL) and dried over anhydrous Na₂SO₄. After filtration, the filtrate was concentrated under reduced pressure. The residue was purified by silica gel column chromatography and eluted with DCM/MeOH (4:1) to afford tert-butyl N-{2-fluoro-4-[(5,6,7,8-tetrahydro-2,6-naphthyridin-3-yl)amino]phenyl}-N-[2-(morpholin-4-yl)ethyl]carbamate (650.00 mg; 60.1%) as a yellow solid.

tert-butyl 7-{7-[(4-{[(tert-butoxy)carbonyl][2-(morpholin-4-yl)ethyl]amino}-3-fluorophenyl)amino]-1,2,3,4-tetrahydro-2,6-naphthyridin-2-yl}-8-methyl-1H,2H,3H-pyrido[2,3-b][1,4]oxazine-1-carboxylate: A solution of tert-butyl N-{2-fluoro-4-[(5,6,7,8-tetrahydro-2,6-naphthyridin-3-yl)amino]phenyl}-N-[2-(morpholin-4-yl)ethyl]carbamate (250.00 mg; 0.52 mmol) and tert-butyl 7-bromo-8-methyl-1H,2H,3H-pyrido[2,3-b][1,4]oxazine-1-carboxylate (285.00 mg; 0.78 mmol) and Pd-PEPPSI-IPentCl 2-methylpyridine (o-picoline) (46.00 mg; 0.05 mmol) and Cs₂CO₃ (356.00 mg; 1.04 mmol) in 1,4-dioxane (10.00 mL) was stirred for 4 h at 100° C. under nitrogen atmosphere. The resulting mixture was concentrated under vacuum. The residue was purified by silica gel column chromatography and eluted with (DCM:MeOH=6:1) to afford tert-butyl 7-{7-[(4-{[(tert-butoxy)carbonyl][2-(morpholin-4-yl)ethyl]amino}-3-fluorophenyl)amino]-1,2,3,4-tetrahydro-2,6-naphthyridin-2-yl}-8-methyl-1H,2H,3H-pyrido[2,3-b][1,4]oxazine-1-carboxylate (100.00 mg; 0.13 mmol; 25.7%) as a yellow solid.

2-fluoro-N4-(6-{8-methyl-1H,2H,3H-pyrido[2,3-b][1,4]oxazin-7-yl}-5,6,7,8-tetrahydro-2,6-naphthyridin-3-yl)-N1-[2-(morpholin-4-yl)ethyl]benzene-1,4-diamine: To a stirred solution of tert-butyl 7-{7-[(4-{[(tert-butoxy)carbonyl][2-(morpholin-4-yl)ethyl]amino}-3-fluorophenyl)amino]-1,2,3,4-tetrahydro-2,6-naphthyridin-2-yl}-8-methyl-1H,2H,3H-pyrido[2,3-b][1,4]oxazine-1-carboxylate (90.00 mg; 0.12 mmol) in DCM (3.00 mL) was added TFA (1.00 mL) dropwise at room temperature. The resulting mixture was stirred for 2 h at room temperature. The mixture was concentrated under reduced pressure. The residue was purified by Prep-HPLC with the follow condition (Column: XBridge Shield RP18 OBD Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 26% B to 56% B in 9 min., 56% B; Wave Length: 254 nm; RT1 (min.): 7). This resulted in 2-fluoro-N4-(6-{8-methyl-1H,2H,3H-pyrido[2,3-b][1,4]oxazin-7-yl}-5,6,7,8-tetrahydro-2,6-naphthyridin-3-yl)-N1-[2-(morpholin-4-yl)ethyl]benzene-1,4-diamine (30.40 mg; 48.5%) as a yellow solid.

HPLC: 99.51% purity, RT=3.26 min. MS: m/z=520.20 [M+H]⁺. ¹H NMR (300 MHz, Methanol-d4) δ 7.86 (s, 1H), 7.24 (s, 1H), 7.17 (dd, J=13.5, 2.4 Hz, 1H), 6.97 (m, J=8.6, 2.4, 1.2 Hz, 1H), 6.75 (dd, J=9.7, 8.6 Hz, 1H), 6.46 (s, 1H), 4.33-4.25 (m, 2H), 3.98 (s, 2H), 3.76-3.67 (m, 4H), 3.45-3.36 (m, 2H), 3.26 (t, J=6.4 Hz, 2H), 3.16 (t, J=5.8 Hz, 2H), 2.86 (t, J=5.8 Hz, 2H), 2.65 (t, J=6.4 Hz, 2H), 2.53 (dd, J=5.5, 3.6 Hz, 4H), 2.12 (s, 3H).

### Example 5: Synthesis of Compound 5 5-chloro-6-(methanesulfonylmethyl)-N-(7-{8-methyl-1H,2H,3H-pyrido[2,3-b][1,4]oxazin-7-yl}-5H,6H,7H,8H-pyrido[3,4-d]pyrimidin-2-yl)pyridin-3-amine

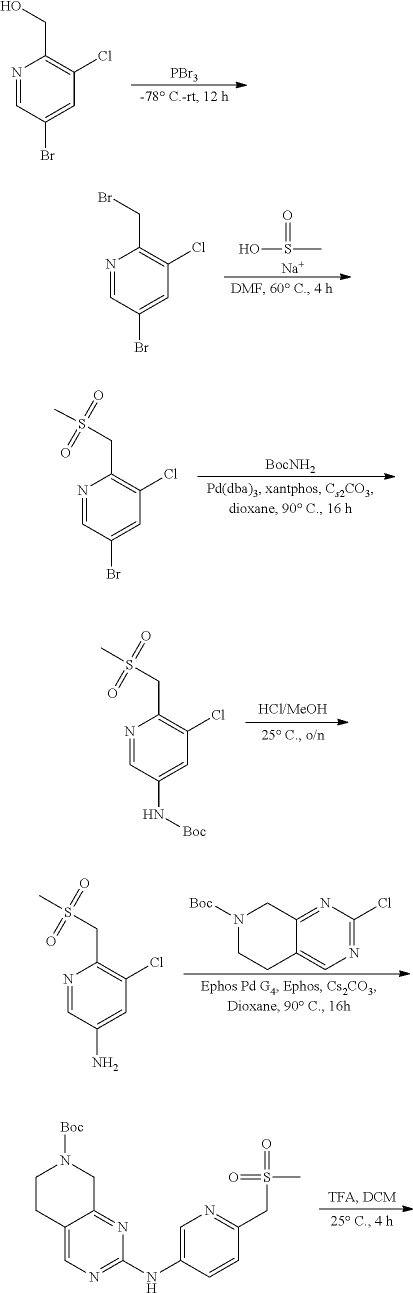

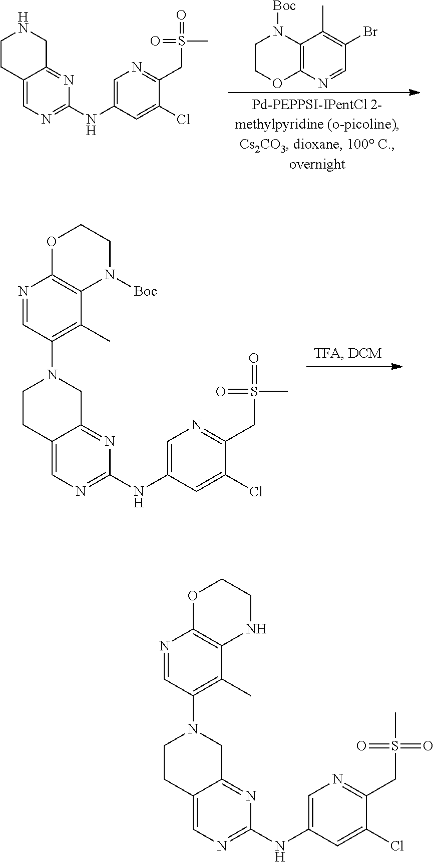 [SMILES: CS(=O)(=O)Cc1ncc(Nc2ncc3c(n2)CNCC3)cc1Cl ; Cc1c(Br)cnc2c1N(C(=O)OC(C)(C)C)CCO2 ; Cc1c(N2CCc3cnc(Nc4cnc(CS(C)(=O)=O)c(Cl)c4)nc3C2)cnc2c1N(C(=O)OC(C)(C)C)CCO2 ; Cc1c(N2CCc3cnc(Nc4cnc(CS(C)(=O)=O)c(Cl)c4)nc3C2)cnc2c1NCCO2]

5-bromo-2-(bromomethyl)-3-chloropyridine: To a stirred mixture of (5-bromo-3-chloropyridin-2-yl) methanol (1.00 g; 3.96 mmol) in DCM (30 mL) was added PBr₃ (0.51 mL; 5.15 mmol) in dropwise at −78° C. Then the mixture was warmed to room temperature and stirred for 12 h at room temperature. The resulting mixture was quenched by ice/water and extracted with EtOAc. The combined organic layers were washed with brine and dried over anhydrous Na₂SO₄. After filtration, the filtrate was concentrated under reduced pressure. The resulting mixture was concentrated under reduced pressure and purified by silica gel column chromatography and eluted with PE/EtOAc (1:1) to afford 5-bromo-2-(bromomethyl)-3-chloropyridine (446.00 mg; 38.1%) as a yellow oil.

5-bromo-3-chloro-2-(methanesulfonylmethyl)pyridine: To a stirred solution of 5-bromo-2-(bromomethyl)-3-chloropyridine (400.00 mg; 1.36 mmol) in DMF (15.00 mL) was added sodium methanesulfinate (281.26 mg; 2.73 mmol). The resulting mixture was stirred for 4 h at 60° C. under nitrogen atmosphere. The resulting mixture was quenched by ice/water and extracted with EtOAc. The combined organic layers were washed with brine and dried over anhydrous Na₂SO₄. After filtration, the filtrate was concentrated under reduced pressure. This resulted in 5-bromo-3-chloro-2-(methanesulfonylmethyl)pyridine (380.00 mg; 94.9%) as a yellow solid.

tert-butyl N-[5-chloro-6-(methanesulfonylmethyl) pyridin-3-yl]carbamate: To a stirred mixture of 5-bromo-3-chloro-2-(methanesulfonylmethyl) pyridine (370.00 mg; 1.14 mmol) and tert-butyl carbamate (210.75 mg; 1.71 mmol; 1.50 eq.) in 1,4-dioxane (15.00 mL) were added Pd₂(dba)₃ (109.83 mg; 0.11 mmol), Xantphos (133.19 mg; 0.23 mmol) and Cs₂CO₃ (781.56 mg; 2.28 mmol) at room temperature. After stirring for 16 h at 90° C. under argon atmosphere, the mixture was concentrated under reduced pressure. The residue was purified by silica gel column chromatography and eluted with DCM/MeOH (19:1) to afford tert-butyl N-[5-chloro-6-(methanesulfonylmethyl) pyridin-3-yl] carbamate (420.00 mg; 90.3%) as a yellow solid.

5-chloro-6-(methanesulfonylmethyl) pyridin-3-amine: A mixture of tert-butyl N-[5-chloro-6-(methanesulfonylmethyl) mpyridin-3-yl] carbamate (410.00 mg; 1.28 mmol) in HCl in MeOH (20.00 mL; 4 M) at 25° C. The resulting mixture was stirred for 12 h at 25° C. under nitrogen atmosphere. The resulting mixture was quenched by ice/water and extracted with EtOAc. The combined organic layers were washed with brine and dried over anhydrous Na₂SO₄. After filtration, the filtrate was concentrated under reduced pressure. This resulted in 5-chloro-6-(methanesulfonylmethyl) pyridin-3-amine (360.00 mg; crude product) as a brown solid.

tert-butyl 2-{[5-chloro-6-(methanesulfonylmethyl) pyridin-3-yl]amino}-5H,6H,7H,8H-pyrido[3,4-d]pyrimidine-7-carboxylate: To a solution of 5-chloro-6-(methanesulfonylmethyl)pyridin-3-amine (300.00 mg; 1.35 mmol) and tert-butyl 2-chloro-5H,6H,7H,8H-pyrido[3,4-d]pyrimidine-7-carboxylate (364.00 mg; 1.35 mmol) in 1,4-dioxane (20.00 mL) were added Ephos (152.00 mg; 0.27 mmol), Cs₂CO₃ (925.00 mg; 2.70 mmol) and EPhos Pd G₄ (131.00 mg; 0.14 mmol). The resulting mixture was stirred for overnight at 90° C. under nitrogen atmosphere. The resulting mixture was concentrated under reduced pressure. The residue was purified by silica gel column chromatography and eluted with DCM/MeOH (24:1) to afford tert-butyl 2-{[5-chloro-6-(methanesulfonylmethyl) pyridin-3-yl]amino}-5H,6H,7H,8H-pyrido[3,4-d]pyrimidine-7-carboxylate (180.00 mg; 25.5%) as a yellow solid.

5-chloro-6-(methanesulfonylmethyl)-N-{5H,6H,7H,8H-pyrido[3,4-d]pyrimidin-2-yl}pyridin-3-amine: To a stirred solution of tert-butyl 2-{[5-chloro-6-(methanesulfonylmethyl) pyridin-3-yl]amino}-5H,6H,7H,8H-pyrido[3,4-d]pyrimidine-7-carboxylate (170.00 mg; 0.33 mmol) in DCM (10.00 mL) was added TFA (2.00 mL) in portions at room temperature. The resulting mixture was stirred for 4 h at 25° C. under nitrogen atmosphere. The mixture was basified to pH [7] with saturated NaHCO₃ (aq.). The resulting mixture was extracted with DCM. The combined organic layers were washed with brine and dried over anhydrous Na₂SO₄. After filtration, the filtrate was concentrated under reduced pressure to afford 5-chloro-6-(methanesulfonylmethyl)-N-{5H,6H,7H,8H-pyrido[3,4-d]pyrimidin-2-yl}pyridin-3-amine (100.00 mg; 81.5%) as a yellow solid.

tert-butyl 7-(2-{[5-chloro-6-(methanesulfonylmethyl) pyridin-3-yl]amino}-5H,6H,7H,8H-pyrido[3,4-d]pyrimidin-7-yl)-8-methyl-1H,2H,3H-pyrido[2,3-b][1,4]oxazine-1-carboxylate: To a solution of 5-chloro-6-(methanesulfonylmethyl)-N-{5H,6H,7H,8H-pyrido[3,4-d]pyrimidin-2-yl}pyridin-3-amine (100.00 mg; 0.26 mmol) and tert-butyl 7-bromo-8-methyl-1H,2H,3H-pyrido[2,3-b][1,4]oxazine-1-carboxylate (86.00 mg; 0.26 mmol) in 1,4-dioxane (2.00 mL) were added Pd-PEPPSI-IPentCl 2-methylpyridine (o-picoline) (23.00 mg; 0.03 mmol) and Cs₂CO₃ (169.00 mg; 0.51 mmol). The resulting mixture was stirred for overnight at 100° C. under nitrogen atmosphere. The resulting mixture was concentrated under reduced pressure. The residue was purified by silica gel column chromatography and eluted with DCM/MeOH (6:1) to afford tert-butyl 7-(2-{[5-chloro-6-(methanesulfonylmethyl) pyridin-3-yl]amino}-5H,6H,7H,8H-pyrido[3,4-d]pyrimidin-7-yl)-8-methyl-1H,2H,3H-pyrido[2,3-b][1,4]oxazine-1-carboxylate (45.00 mg; 21.8%) as a yellow solid.

5-chloro-6-(methanesulfonylmethyl)-N-(7-{8-methyl-1H,2H,3H-pyrido[2,3-b][1,4]oxazin-7-yl}-5H,6H,7H,8H-pyrido[3,4-d]pyrimidin-2-yl)pyridin-3-amine: To a stirred solution of tert-butyl 7-(2-{[5-chloro-6-(methanesulfonylmethyl) pyridin-3-yl]amino}-5H,6H,7H,8H-pyrido[3,4-d]pyrimidin-7-yl)-8-methyl-1H,2H,3H-pyrido[2,3-b][1,4]oxazine-1-carboxylate (40.00 mg; 0.05 mmol) in DCM (5.00 mL) was added TFA (1.00 mL) in portions at room temperature. The resulting mixture was stirred for 4 h at 25° C. under nitrogen atmosphere. The mixture was basified to pH=7 with saturated NaHCO₃ (aq.). The resulting mixture was extracted with DCM. The combined organic layers were washed with brine and dried over anhydrous Na₂SO₄. After filtration, the filtrate was concentrated under reduced pressure. The residue was purified by Prep-HPLC with the following conditions (Column: XBridge Shield RP18 OBD Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 18% B to 48% B in 9 min., 48% B; Wave Length: 254 nm; RT1 (min.): 7) to afford 5-chloro-6-(methanesulfonylmethyl)-N-(7-{8-methyl-1H,2H,3H-pyrido[2,3-b][1,4]oxazin-7-yl}-5H,6H,7H,8H-pyrido[3,4-d]pyrimidin-2-yl)pyridin-3-amine (4.60 mg; 18.1%) as a white solid.

HPLC: 97.69% purity, RT=2.63 min. MS: m/z=502.00 [M+H]⁺. ¹H NMR (300 MHz, DMSO-d6) δ 10.15 (s, 1H), 8.87 (d, J=2.3 Hz, 1H), 8.50 (d, J=2.3 Hz, 1H), 8.46 (s, 1H), 7.30 (s, 1H), 5.53 (s, 1H), 4.67 (s, 2H), 4.30-4.11 (m, 2H), 4.02 (s, 2H), 3.32 (s, 2H), 3.13 (d, J=5.5 Hz, 2H), 3.06 (s, 3H), 2.85 (d, J=5.8 Hz, 2H), 2.05 (s, 3H).

### Example 6: Synthesis of Compound 6 2-fluoro-N4-(7-{8-methyl-1H,2H,3H-pyrido[2,3-b][1,4]oxazin-7-yl}-5H,6H,7H,8H-pyrido[3,4-d]pyrimidin-2-yl)-N1-[2-(morpholin-4-yl)ethyl]benzene-1,4-diamine

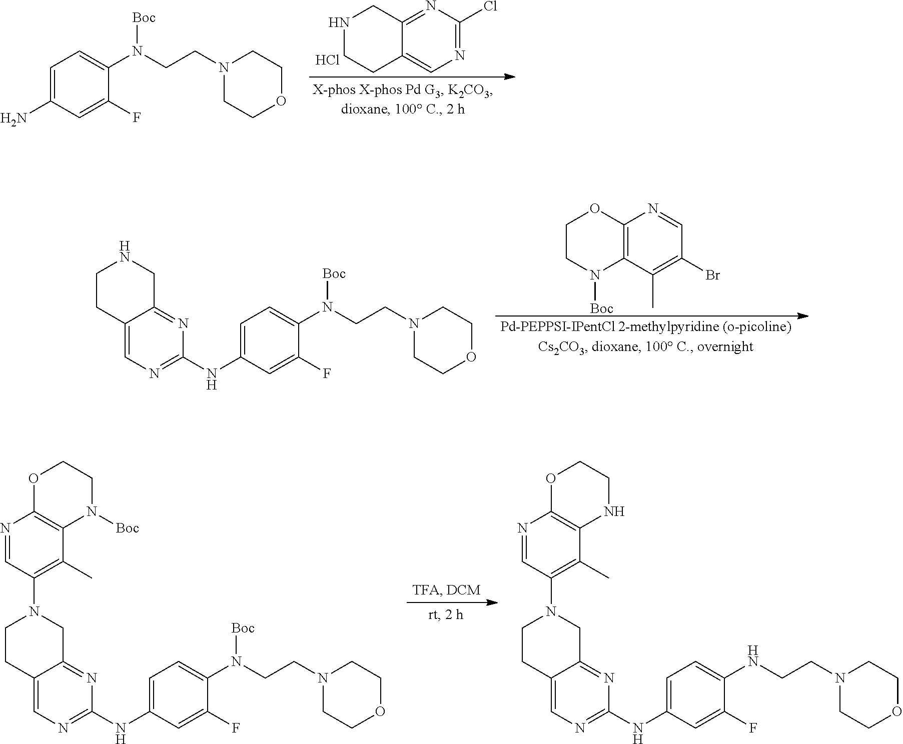

tert-butyl N-[2-fluoro-4-({5H,6H,7H,8H-pyrido[3,4-d]pyrimidin-2-yl}amino)phenyl]-N-[2-(morpholin-4-yl)ethyl]carbamate: To a stirred solution of tert-butyl N-(4-amino-2-fluorophenyl)-N-[2-(morpholin-4-yl)ethyl]carbamate (400.00 mg; 1.18 mmol) and 2-chloro-5H,6H,7H,8H-pyrido[3,4-d]pyrimidine (267.00 mg; 1.53 mmol) in 1,4-dioxane (5.00 mL) were added EPhos Pd G₄ (76.00 mg; 0.08 mmol), Ephos (63.00 mg; 0.11 mmol) and Cs₂CO₃ (1.20 g; 3.50 mmol) in portions at room temperature under N₂ atmosphere. The resulting mixture was stirred for 2 h at 100° C. under N₂ atmosphere. The resulting mixture was diluted with water (50 mL), extracted with EtOAc (3×50 mL). The combined organic layers were washed with brine 50 mL) and dried over anhydrous Na₂SO₄. The resulting mixture was concentrated under reduced pressure. The residue was purified by silica gel column and eluted with 15% MeOH in DCM to afford tert-butyl N-[2-fluoro-4-({5H,6H,7H,8H-pyrido[3,4-d]pyrimidin-2-yl}amino)phenyl]-N-[2-(morpholin-4-yl)ethyl]carbamate (340.00 mg; 53.6%) as an off-white solid.

tert-butyl 7-{2-[(4-{[(tert-butoxy)carbonyl][2-(morpholin-4-yl)ethyl]amino}-3-fluorophenyl)amino]-5H,6H,7H,8H-pyrido[3,4-d]pyrimidin-7-yl}-8-methyl-1H,2H,3H-pyrido[2,3-b][1,4]oxazine-1-carboxylate: To a stirred solution of tert-butyl N-[2-fluoro-4-({5H,6H,7H,8H-pyrido[3,4-d]pyrimidin-2-yl}amino)phenyl]-N-[2-(morpholin-4-yl)ethyl]carbamate (320.00 mg; 0.59 mmol) and tert-butyl 7-bromo-8-methyl-1H,2H,3H-pyrido[2,3-b][1,4]oxazine-1-carboxylate (267.00 mg; 0.81 mmol) in 1,4-dioxane (5.00 mL) were added Pd-PEPPSI-IPentCl 2-methylpyridine (o-picoline) (57.00 mg; 0.06 mmol) and Cs₂CO₃ (664.00 mg; 1.94 mmol; 3.25 eq.) in portions at room temperature under N₂ atmosphere. The resulting mixture was stirred for overnight at 100° C. under N₂ atmosphere. The resulting mixture was diluted with water (50 mL), extracted with EtOAc (3×50 mL). The combined organic layers were washed with brine (50 mL) and dried over anhydrous Na₂SO₄. The resulting mixture was concentrated under reduced pressure. The residue was purified by silica gel column and eluted with 100% EtOAc to afford tert-butyl 7-{2-[(4-{[(tert-butoxy) carbonyl][2-(morpholin-4-yl)ethyl]amino}-3-fluorophenyl) amino]-5H,6H,7H,8H-pyrido[3,4-d]pyrimidin-7-yl}-8-methyl-1H,2H,3H-pyrido[2,3-b][1,4]oxazine-1-carboxylate (280.00 mg; 60.4%) as a yellow solid.

2-fluoro-N4-(7-{8-methyl-1H,2H,3H-pyrido[2,3-b][1,4]oxazin-7-yl}-5H,6H,7H,8H-pyrido[3,4-d]pyrimidin-2-yl)-N1-[2-(morpholin-4-yl)ethyl]benzene-1,4-diamine: To a stirred solution of tert-butyl 7-{2-[(4-{[(tert-butoxy)carbonyl][2-(morpholin-4-yl)ethyl]amino}-3-fluorophenyl)amino]-5H,6H,7H,8H-pyrido[3,4-d]pyrimidin-7-yl}-8-methyl-1H,2H,3H-pyrido[2,3-b][1,4]oxazine-1-carboxylate (260.00 mg; 0.33 mmol) in DCM (3.00 mL) was added TFA (1.00 mL) dropwise at 0° C. under N₂ atmosphere. The resulting mixture was stirred for 2 h at room temperature under N₂ atmosphere. The resulting mixture was concentrated under reduced pressure. The residue was purified by Prep-HPLC with the following condition: (Column: XBridge Shield RP18 OBD Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 26% B to 56% B in 9 min., 56% B; Wave Length: 254 nm; RT1 (min.): 7) to afford 2-fluoro-N4-(7-{8-methyl-1H,2H,3H-pyrido[2,3-b][1,4]oxazin-7-yl}-5H,6H,7H,8H-pyrido[3,4-d]pyrimidin-2-yl)-N1-[2-(morpholin-4-yl)ethyl]benzene-1,4-diamine (37.60 mg; 19.5%) as a yellow solid.

HPLC: 90.02% purity, RT=2.17 min. MS: m/z=521.15 [M+H]⁺. ¹H NMR (300 MHz, DMSO-d6) δ 9.28 (s, 1H), 8.27 (s, 1H), 7.59 (dd, J=14.5, 2.4 Hz, 1H), 7.26 (d, J=9.0 Hz, 2H), 6.74-6.62 (m, 1H), 5.53 (s, 1H), 4.77 (s, 1H), 4.19 (s, 2H), 3.92 (s, 2H), 3.58 (t, J=4.6 Hz, 4H), 3.30 (s, 2H), 3.14 (dd, J=12.6, 6.3 Hz, 4H), 2.76 (s, 2H), 2.54 (s, 2H), 2.40 (t, J=4.6 Hz, 4H), 2.03 (s, 3H).

### Example 7: Synthesis of Compound 7 2-[(6-{8-methyl-1H,2H,3H-pyrido[2,3-b]oxazin-7-yl}-5,6,7,8-tetrahydro-2,6-naphthyridin-3-yl) amino]-6-(propan-2-yl)-4H,5H,6H,7H,8H-pyrazolo[1,5-d][1,4]diazepin-7-one

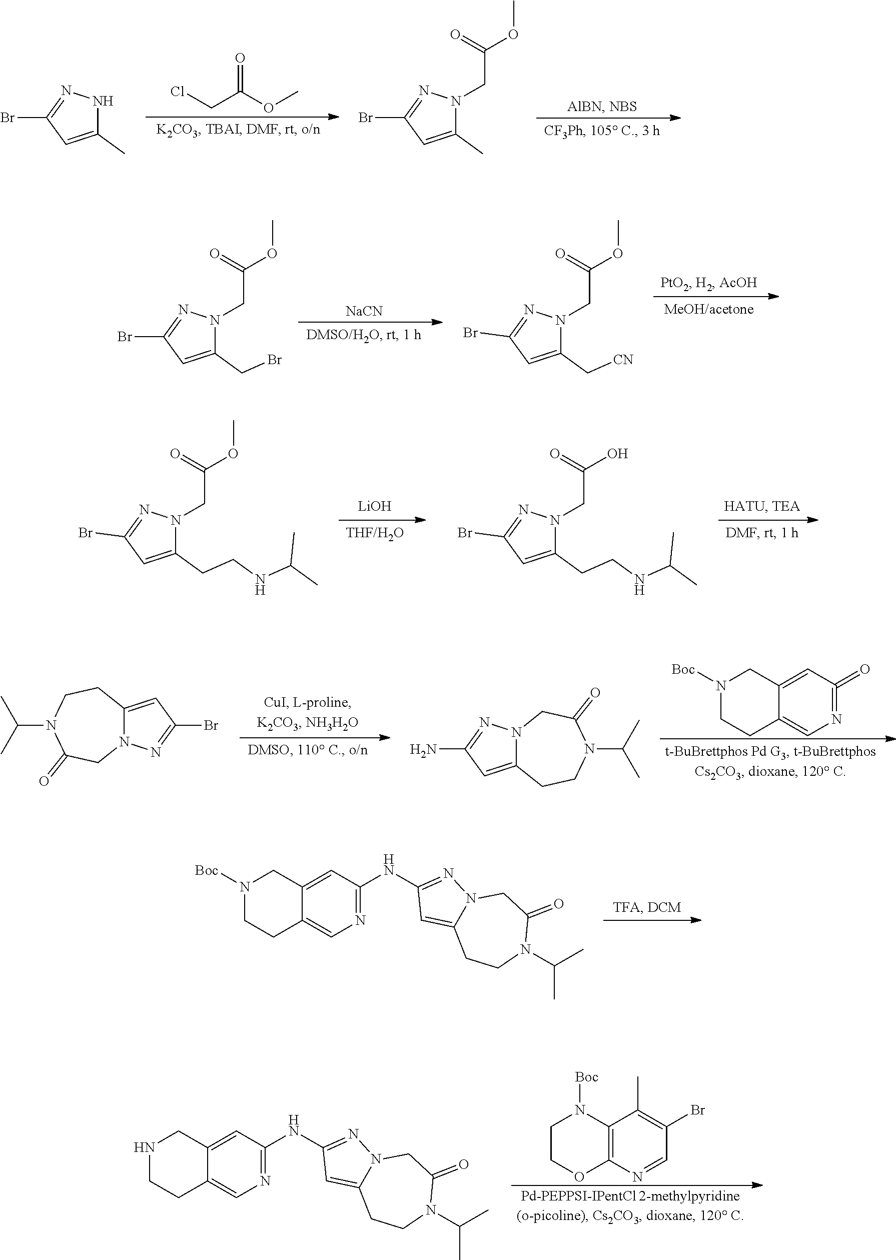

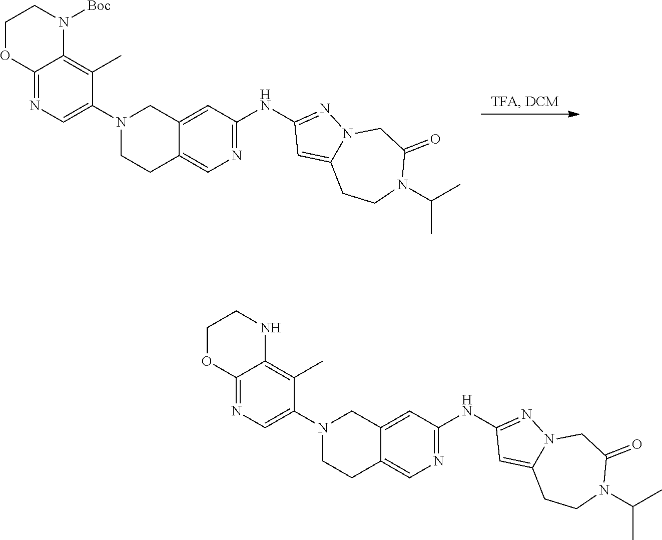 [SMILES: Cc1c(N2CCc3cnc(Nc4cc5n(n4)CC(=O)N(C(C)C)CC5)cc3C2)cnc2c1N(C(=O)OC(C)(C)C)CCO2 ; Cc1c(N2CCc3cnc(Nc4cc5n(n4)CC(=O)N(C(C)C)CC5)cc3C2)cnc2c1NCCO2]

methyl 2-(3-bromo-5-methyl-1H-pyrazol-1-yl)acetate: To a stirred solution of 3-bromo-5-methyl-1H-pyrazole (20.00 g; 118.01 mmol), tetra-n-butylammonium iodide (TBAI) (2.40 g; 6.17 mmol) and K₂CO₃ (31.10 g; 213.78 mmol) in DMF (200.00 mL) was added methyl 2-chloroacetate (20.30 g; 177.70 mmol) dropwise at room temperature under N₂ atmosphere. The resulting mixture was stirred for overnight at room temperature under N₂ atmosphere. The resulting mixture was diluted with water (1.5 L), extracted with EtOAc (500 mL×3). The combined organic layers were washed with brine (1 L) and dried over anhydrous Na₂SO₄. The resulting mixture was concentrated under reduced pressure. The residue was purified by silica gel column and eluted with 30% EA in PE to afford methyl 2-(3-bromo-5-methyl-1H-pyrazol-1-yl)acetate (29.00 g; 98.5%).

methyl 2-[3-bromo-5-(bromomethyl)-1H-pyrazol-1-yl]acetate: To a stirred solution of methyl 2-(3-bromo-5-methyl-1H-pyrazol-1-yl)acetate (25.00 g; 107.27 mmol) and NBS (20.10 g; 107.27 mmol) in PhCF₃ (500.00 mL) was added 2-[(1Z)-2-(1-cyano-1-methylethyl)diazen-1-yl]-2-methylpropanenitrile (18.54 g; 107.27 mmol) in portions at room temperature under N₂ atmosphere. The resulting mixture was stirred for 3 h at 105° C. under N₂ atmosphere. The reaction mixture was diluted with water (1 L) and extracted with DCM (3×400 mL). The combined organic layers were washed with brine (500 mL) and dried over anhydrous Na₂SO₄ and concentrated under reduced pressure. The residue was purified by silica gel column and eluted with EtOAc:PE=8:1 to afford methyl 2-[3-bromo-5-(bromomethyl)-1H-pyrazol-1-yl]acetate (22.00 g; 46.85%).

methyl 2-[3-bromo-5-(cyanomethyl)-1H-pyrazol-1-yl]acetate: To a stirred solution of methyl 2-[3-bromo-5-(bromomethyl)-1H-pyrazol-1-yl]acetate (22.00 g; 50.25 mmol) in DMSO (200.00 mL) were added sodium cyanide (3.77 g; 75.38 mmol) and water (4.00 mL; 219.76 mmol) in portions at room temperature under N₂ atmosphere. The resulting mixture was stirred for 1 h at 25° C. under N₂ atmosphere. The resulting mixture was quenched with FeSO₄ (aq.), extracted with EtOAc (3×100 mL). The combined organic layers were washed with brine (15 mL) and dried over anhydrous Na₂SO₄. The resulting mixture was concentrated under reduced pressure. The residue was purified by silica gel column and eluted with PE:EA=7:1 and concentrated to afford methyl 2-[3-bromo-5-(cyanomethyl)-1H-pyrazol-1-yl]acetate (8.50 g; 57.01%) as a yellow solid.

methyl 2-(3-bromo-5-{2-[(propan-2-yl) amino]ethyl}-1H-pyrazol-1-yl)acetate: To a stirred mixture of methyl 2-[3-bromo-5-(cyanomethyl)-1H-pyrazol-1-yl]acetate (20.00 g; 58.08 mmol) in MeOH (50.00 mL) and acetone (12.50 mL) was added dioxoplatinum (6.00 g; 25.10 mmol) and AcOH (60.00 mg; 0.99 mmol) dropwise at room temperature under nitrogen. The resulting mixture was stirred for 1.5 h at room temperature under hydrogen (11.71 g; 5807.68 mmol) atmosphere. The resulting mixture was filtered and concentrated under reduced pressure. The residue was purified by silica gel column and eluted with DCM:MeOH=20:1 to afford methyl 2-(3-bromo-5-{2-[(propan-2-yl) amino]ethyl}-1H-pyrazol-1-yl)acetate (13.00 g; 64.99%) as a yellow solid.

2-(3-bromo-5-{2-[(propan-2-yl) amino]ethyl}-1H-pyrazol-1-yl) acetic acid: To a stirred mixture of methyl 2-(3-bromo-5-{2-[(propan-2-yl) amino]ethyl}-1H-pyrazol-1-yl)acetate (7.00 g; 58.07 mmol) in THF (100.00 mL) was added LiOH (5.13 g; 116.14 mmol) in water (200.00 mL) dropwise at room temperature. The resulting mixture was stirred for 1 h at room temperature under nitrogen atmosphere. The resulting mixture was adjusted PH=5-6 with HCl (6N) and concentrated under reduced pressure to afford 2-(3-bromo-5-{2-[(propan-2-yl) amino]ethyl}-1H-pyrazol-1-yl) acetic acid (30.00 g; crude product) as a white solid, which was used for next step without further purification.

2-bromo-6-(propan-2-yl)-4H,5H,6H,7H,8H-pyrazolo[1,5-d][1,4]diazepin-7-one: To a stirred solution of 2-(3-bromo-5-{2-[(propan-2-yl)amino]ethyl}-1H-pyrazol-1-yl)acetic acid (25.00 g; 74.24 mmol) and HATU (29.71 g; 74.24 mmol) in DMF (250.00 mL) was added TEA (16.29 mL; 111.35 mmol) in portions at room temperature under N₂ atmosphere. The resulting mixture was stirred for 1 h at ° C. under N₂ atmosphere. The resulting mixture was diluted with water (1 L), extracted with EtOAc (3×300 mL). The combined organic layers were washed with brine (500 mL) and dried over anhydrous Na₂SO₄. The resulting mixture was concentrated under reduced pressure. The residue was slurry with PE:EtOAc=2:1 (30 mL) and the solid was filtered to afford 2-bromo-6-(propan-2-yl)-4H,5H,6H,7H,8H-pyrazolo[1,5-d][1,4]diazepin-7-one (11.70 g; 54.55%) as a white solid.

2-amino-6-(propan-2-yl)-4H,5H,6H,7H,8H-pyrazolo[1,5-d][1,4]diazepin-7-one: To a stirred solution of 2-bromo-6-(propan-2-yl)-4H,5H,6H,7H,8H-pyrazolo[1,5-d][1,4]diazepin-7-one (1.00 g; 3.46 mmol) and NH₃—H₂O (2.00 mL; 759.13 mmol) in DMSO (10.00 mL) was added CuI (139.00 mg; 0.69 mmol), L-proline (168.00 mg; 1.39 mmol) and K₂CO₃ (1008.00 mg; 6.93 mmol). The resulting mixture was stirred for overnight at 110° C. under nitrogen atmosphere using a sealed tube. The residue was purified by reverse flash chromatography with the following conditions: column, C18 silica gel; mobile phase, MeCN in water, 10% to 50% gradient in 10 min.; detector, UV 254 nm. This resulted in 2-amino-6-(propan-2-yl)-4H,5H,6H,7H,8H-pyrazolo[1,5-d][1,4]diazepin-7-one (380.00 mg; 52.7%) as a yellow solid.

tert-butyl 7-{[7-oxo-6-(propan-2-yl)-4H,5H,6H,7H,8H-pyrazolo[1,5-d][1,4]diazepin-2-yl]amino}-1,2,3,4-tetrahydro-2,6-naphthyridine-2-carboxylate: To a stirred solution of 2-amino-6-(propan-2-yl)-4H,5H,6H,7H,8H-pyrazolo[1,5-d][1,4]diazepin-7-one (330.00 mg; 1.51 mmol) in 1,4-dioxane (6.00 mL) were added tert-butyl 7-chloro-1,2,3,4-tetrahydro-2,6-naphthyridine-2-carboxylate (468.42 mg; 1.66 mmol), EPhos Pd G₄ (145.55 mg; 0.15 mmol), Ephos (84.74 mg; 0.15 mmol) and Cs₂CO₃ (1548.84 mg; 4.52 mmol) in portions at room temperature under N₂ atmosphere. The resulting mixture was stirred for 3 h at 120° C. under N₂ atmosphere. The residue was purified by silica gel column and eluted with 91% EtOAc in PE to afford tert-butyl 7-{[7-oxo-6-(propan-2-yl)-4H,5H,6H,7H,8H-pyrazolo[1,5-d][1,4]diazepin-2-yl]amino}-1,2,3,4-tetrahydro-2,6-naphthyridine-2-carboxylate (200.00 mg; 29.9%) as a white solid.

6-(propan-2-yl)-2-[(5,6,7,8-tetrahydro-2,6-naphthyridin-3-yl) amino]-4H,5H,6H,7H,8H-pyrazolo[1,5-d][1,4]diazepin-7-one: To a stirred solution of tert-butyl 7-{[7-oxo-6-(propan-2-yl)-4H,5H,6H,7H,8H-pyrazolo[1,5-d][1,4]diazepin-2-yl]amino}-1,2,3,4-tetrahydro-2,6-naphthyridine-2-carboxylate (190.00 mg; 1.00 eq.) in DCM (2.40 mL) was added TFA (0.80 mL) in portions at 0° C. under N₂ atmosphere. The resulting mixture was stirred for 1 h at room temperature under N₂ atmosphere. The resulting mixture was adjusted pH to 10 with NaHCO₃ (50 mL), extracted with DCM (3×50 mL). The combined organic layers were washed with brine (15 mL) and dried over anhydrous Na₂SO₄. The resulting mixture was concentrated under reduced pressure to afford 6-(propan-2-yl)-2-[(5,6,7,8-tetrahydro-2,6-naphthyridin-3-yl)amino]-4H,5H,6H,7H,8H-pyrazolo[1,5-d][1,4]diazepin-7-one (140.00 mg; crude product) as a brown solid.

tert-butyl 8-methyl-7-(7-{[7-oxo-6-(propan-2-yl)-4H,5H,6H,7H,8H-pyrazolo[1,5-d][1,4]diazepin-2-yl]amino}-1,2,3,4-tetrahydro-2,6-naphthyridin-2-yl)-1H,2H,3H-pyrido[2,3-b][1,4]oxazine-1-carboxylate: To a stirred solution of 6-(propan-2-yl)-2-[(5,6,7,8-tetrahydro-2,6-naphthyridin-3-yl)amino]-4H,5H,6H,7H,8H-pyrazolo[1,5-d][1,4]diazepin-7-one (110.00 mg; 0.32 mmol) in 1,4-dioxane (3.00 mL) were added tert-butyl 7-bromo-8-methyl-1H,2H,3H-pyrido[2,3-b][1,4]oxazine-1-carboxylate (221.70 mg; 0.64 mmol), Pd-PEPPSI-IPentCl 2-methylpyridine (o-picoline) (28.29 mg; 0.03 mmol) and Cs₂CO₃ (329.14 mg; 0.96 mmol) in portions at room temperature under N₂ atmosphere. The residue was purified by silica gel column and eluted with 75% EtOAc in PE to afford crude product. The crude product was repurified by C18 flash column and eluted with 55% ACN in water to afford tert-butyl 8-methyl-7-(7-{[7-oxo-6-(propan-2-yl)-4H,5H,6H,7H,8H-pyrazolo[1,5-d][1,4]diazepin-2-yl]amino}-1,2,3,4-tetrahydro-2,6-naphthyridin-2-yl)-1H,2H,3H-pyrido[2,3-b][1,4]oxazine-1-carboxylate (120.00 mg; 57.4%) as a brown oil.

2-[(6-{8-methyl-1H,2H,3H-pyrido[2,3-b][1,4]oxazin-7-yl}-5,6,7,8-tetrahydro-2,6-naphthyridin-3-yl) amino]-6-(propan-2-yl)-4H,5H,6H,7H,8H-pyrazolo[1,5-d][1,4]diazepin-7-one: To a stirred solution of tert-butyl 8-methyl-7-(7-{[7-oxo-6-(propan-2-yl)-4H,5H,6H,7H,8H-pyrazolo[1,5-d][1,4]diazepin-2-yl]amino}-1,2,3,4-tetrahydro-2,6-naphthyridin-2-yl)-1H,2H,3H-pyrido[2,3-b][1,4]oxazine-1-carboxylate (115.00 mg; 0.195 mmol) in DCM (1.50 mL) was added TFA (0.50 mL) in portions at 0° C. under N₂ atmosphere. The resulting mixture was stirred for 1 h at room temperature under N₂ atmosphere. The residue was purified by Pre-HPLC (Column: X Bridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 25% B to 55% B in 9 min., 55% B; Wave Length: 254 nm; RTT (min.): 7) to afford 2-[(6-{8-methyl-1H,2H,3H-pyrido[2,3-b][1,4]oxazin-7-yl}-5,6,7,8-tetrahydro-2,6-naphthyridin-3-yl) amino]-6-(propan-2-yl)-4H,5H,6H,7H,8H-pyrazolo[1,5-d][1,4]diazepin-7-one (24.30 mg; 24.70%) as a white solid.

HPLC: 96.17% purity, RT=2.445 min. MS: m/z=489.30 [M+H]⁺. ¹H NMR (400 MHz, DMSO-d6) δ 8.99 (s, 1H), 7.93 (s, 1H), 7.26 (s, 1H), 7.00 (s, 1H), 6.17 (s, 1H), 5.49 (s, 1H), 4.90 (s, 2H), 4.58 (m, J=6.8 Hz, 1H), 4.21-4.15 (m, 2H), 3.97 (s, 2H), 3.76 (d, J=11.7 Hz, 2H), 3.33 (s, 2H), 3.07 (s, 2H), 2.96 (t, J=5.9 Hz, 2H), 2.79 (s, 2H), 2.02 (s, 3H), 1.11 (d, J=6.8 Hz, 6H).

### Example 8: Synthesis of Compound 8 3-methyl-N-{5-[(7-{8-methyl-1H,2H,3H-pyrido[2,3-b][1,4]oxazin-7-yl}-5H,6H,7H,8H-pyrido[3,4-d]pyrimidin-2-yl) amino]pyridin-2-yl}oxetane-3-carboxamide

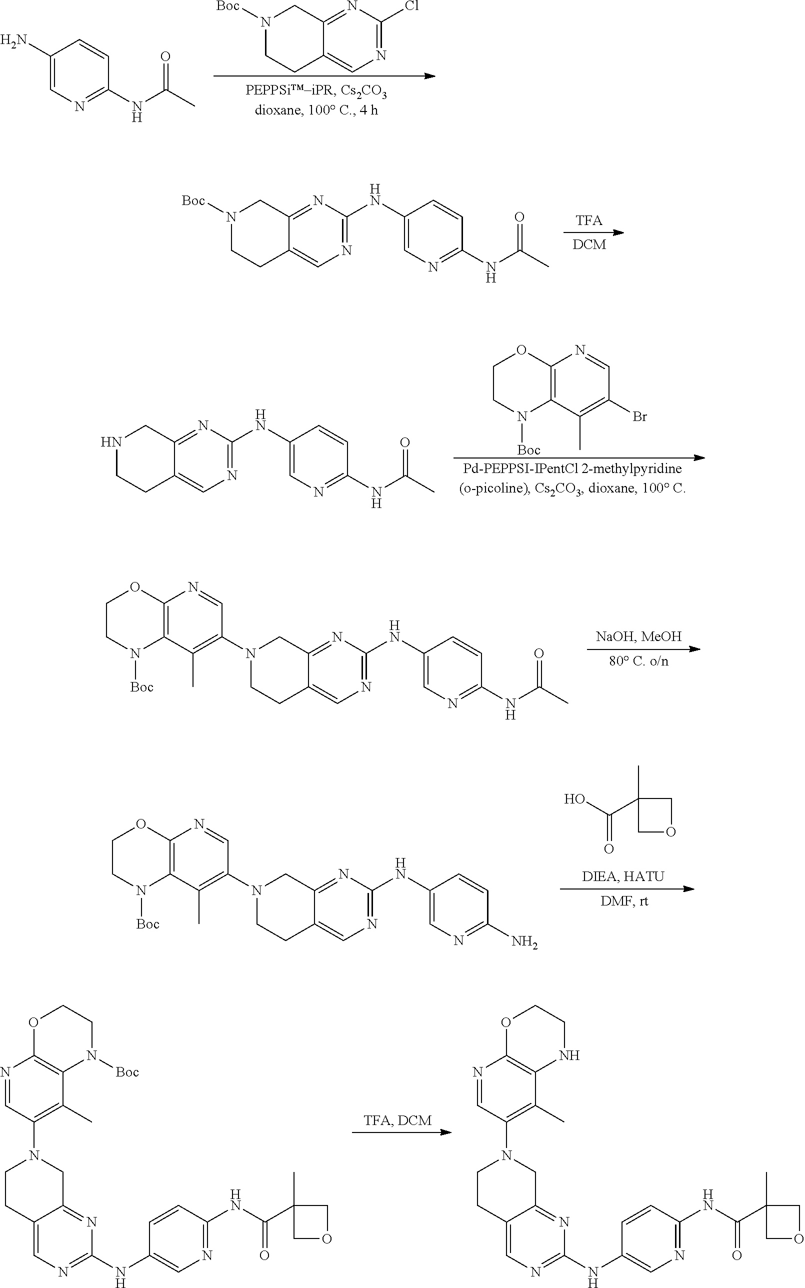

To a stirred solution of tert-butyl 2-chloro-5H,6H,7H,8H-pyrido[3,4-d]pyrimidine-7-carboxylate (1037.00 mg; 3.77 mmol) and N-(5-aminopyridin-2-yl) acetamide (500.00 mg; 3.14 mmol) in 1,4-dioxane (50.00 mL) were added PEPPSI™-IPr (269.00 mg; 0.31 mmol) and Cs₂CO₃ (2150.00 mg; 6.27 mmol) in portions at room temperature under N₂ atmosphere. The resulting mixture was stirred for 4 h at 100° C. under N₂ atmosphere. The resulting mixture was concentrated under vacuum. The residue was purified by silica gel column chromatography and eluted with (MeOH:DCM=1:9) to afford tert-butyl 2-[(6-acetamidopyridin-3-yl) amino]-5H,6H,7H,8H-pyrido[3,4-d]pyrimidine-7-carboxylate (740.00 mg; 61.3%) as a yellow solid.

N-[5-({5H,6H,7H,8H-pyrido[3,4-d]pyrimidin-2-yl}amino) pyridin-2-yl]acetamide: A solution of tert-butyl 2-[(6-acetamidopyridin-3-yl) amino]-5H,6H,7H,8H-pyrido[3,4-d]pyrimidine-7-carboxylate (720.00 mg; 1.87 mmol) and TFA (3.00 mL) in DCM (9.00 mL) was stirred for 1 h at room temperature under nitrogen atmosphere. The mixture was adjusted to pH 12 with NaOH (aq.) and extracted with EtOAc. The combined organic layers were washed with brine and dried over anhydrous Na₂SO₄. After filtration, the filtrate was concentrated under reduced pressure. This resulted in N-[5-({5H,6H,7H,8H-pyrido[3,4-d]pyrimidin-2-yl}amino) pyridin-2-yl]acetamide (280.00 mg; 52.6%) as a yellow solid.

tert-butyl 7-{2-[(6-acetamidopyridin-3-yl) amino]-5H,6H,7H,8H-pyrido[3,4-d]pyrimidin-7-yl}-8-methyl-1H,2H,3H-pyrido[2,3-b][1,4]oxazine-1-carboxylate: A solution of N-[5-({5H,6H,7H,8H-pyrido[3,4-d]pyrimidin-2-yl}amino) pyridin-2-yl]acetamide (260.00 mg; 0.91 mmol) and tert-butyl 7-bromo-8-methyl-1H,2H,3H-pyrido[2,3-b][1,4]oxazine-1-carboxylate (402.00 mg; 1.10 mmol) and Pd-PEPPSI-IPentCl 2-methylpyridine (o-picoline) (81.00 mg; 0.09 mmol) and Cs₂CO₃ (627.00 mg; 1.83 mmol) in 1,4-dioxane (13.00 mL) was stirred for 4 h at 100° C. under nitrogen atmosphere. The resulting mixture was concentrated under vacuum. The residue was purified by silica gel column chromatography and eluted with (DCM:MeOH=8:2) to afford tert-butyl 7-{2-[(6-acetamidopyridin-3-yl) amino]-5H,6H,7H,8H-pyrido[3,4-d]pyrimidin-7-yl}-8-methyl-1H,2H,3H-pyrido[2,3-b][1,4]oxazine-1-carboxylate (460.00 mg; 94.4%) as a yellow solid.

tert-butyl 7-{2-[(6-aminopyridin-3-yl) amino]-5H,6H,7H,8H-pyrido[3,4-d]pyrimidin-7-yl}-8-methyl-1H,2H,3H-pyrido[2,3-b][1,4]oxazine-1-carboxylate: A solution of tert-butyl 7-{2-[(6-acetamidopyridin-3-yl) amino]-5H,6H,7H,8H-pyrido[3,4-d]pyrimidin-7-yl}-8-methyl-1H,2H,3H-pyrido[2,3-b][1,4]oxazine-1-carboxylate (420.00 mg; 0.79 mmol) and NaOH (330.00 mg; 7.84 mmol) in MeOH (8.00 mL) was stirred for 10 h at 80° C. under nitrogen atmosphere. The resulting mixture was concentrated under vacuum. The residue was purified by reverse flash chromatography with the following conditions: column, C18 silica gel; mobile phase, H₂O/MeCN in water, 10% to 50% gradient in 10 min.; detector, UV 254 nm. This resulted in tert-butyl 7-{2-[(6-aminopyridin-3-yl) amino]-5H,6H,7H,8H-pyrido[3,4-d]pyrimidin-7-yl}-8-methyl-1H,2H,3H-pyrido[2,3-b][1,4]oxazine-1-carboxylate (280.00 mg; 72.4%) as an off-white solid.

tert-butyl 8-methyl-7-(2-{[6-(3-methyloxetane-3-amido) pyridin-3-yl]amino}-5H,6H,7H,8H-pyrido[3,4-d]pyrimidin-7-yl)-1H,2H,3H-pyrido[2,3-b]oxazine-1-carboxylate: To a stirred solution of tert-butyl 7-{2-[(6-aminopyridin-3-yl) amino]-5H,6H,7H,8H-pyrido[3,4-d]pyrimidin-7-yl}-8-methyl-1H,2H,3H-pyrido[2,3-b][1,4]oxazine-1-carboxylate (260.00 mg; 0.53 mmol), N,N-diisopropylethylamine (DIEA) (0.29 mL; 1.56 mmol), 3-methyloxetane-3-carboxylic acid (33.00 mg; 0.27 mmol) in DMF (7.00 mL) was added hexafluorophosphate azabenzotriazole tetramethyl uronium (HATU) (355.00 mg; 0.89 mmol) at room temperature under N₂ atmosphere. The resulting mixture was stirred for overnight at room temperature. The reaction was quenched with water at room temperature. The resulting mixture was extracted with EtOAc (3×100 mL). The combined organic layers were washed with brine (1×50 mL) and dried over anhydrous Na₂SO₄. After filtration, the filtrate was concentrated under reduced pressure. The residue was purified by silica gel column chromatography and eluted with DCM/MeOH (3:1) to afford tert-butyl 8-methyl-7-(2-{[6-(3-methyloxetane-3-amido) pyridin-3-yl]amino}-5H,6H,7H,8H-pyrido[3,4-d]pyrimidin-7-yl)-1H,2H,3H-pyrido[2,3-b][1,4]oxazine-1-carboxylate (200.00 mg; 61.5%) as a yellow solid.

3-methyl-N-{5-[(7-{8-methyl-1H,2H,3H-pyrido[2,3-b][1,4]oxazin-7-yl}-5H,6H,7H,8H-pyrido[3,4-d]pyrimidin-2-yl) amino]pyridin-2-yl}oxetane-3-carboxamide: To a stirred solution of tert-butyl 8-methyl-7-(2-{[6-(3-methyloxetane-3-amido) pyridin-3-yl]amino}-5H,6H,7H,8H-pyrido[3,4-d]pyrimidin-7-yl)-1H,2H,3H-pyrido[2,3-b][1,4]oxazine-1-carboxylate (180.00 mg; 0.29 mmol) in DCM (9.00 mL) was added TFA (3.00 mL) dropwise at room temperature. The resulting mixture was stirred for 3 h at room temperature. The resulting mixture was purified by Prep-HPLC with the following condition (Column: Xselect CSH C18 OBD Column 30*150 mm, 5 μm, n; Mobile Phase A: water (0.1% FA), Mobile Phase B: ACN; Flow rate: 60 mL/min.; Gradient: 3% B to 25% B in 8 min, 25% B; Wavelength: 254/220 nm; RT1 (min.): 7.4). This resulted in 3-methyl-N-{5-[(7-{8-methyl-1H,2H,3H-pyrido[2,3-b][1,4]oxazin-7-yl}-5H,6H,7H,8H-pyrido[3,4-d]pyrimidin-2-yl) amino]pyridin-2-yl}oxetane-3-carboxamide (13.40 mg; 9.2%) as a yellow solid.

HPLC: 98.38% purity, RT=2.47 min. MS: m/z=489.10 [M+H]⁺. ¹H NMR (300 MHz, DMSO-d6) δ 9.84 (s, 1H), 8.66 (d, J=2.4 Hz, 1H), 8.39 (s, 1H), 8.23 (s, 1H), 7.91 (dd, J=9.5, 2.4 Hz, 1H), 7.30 (s, 1H), 6.92 (d, J=9.5 Hz, 1H), 5.55 (s, 1H), 4.36 (d, J=14.0 Hz, 1H), 4.23-4.11 (m, 3H), 4.01 (s, 2H), 3.46 (d, J=10.8 Hz, 2H), 3.33 (d, J=7.9 Hz, 2H), 3.13 (s, 2H), 2.82 (s, 2H), 2.04 (s, 3H), 1.00 (s, 3H).

### Example 9: Synthesis of Compound 9 2-(azetidin-1-yl)-N-{2-methyl-4-[(6-{8-methyl-1H,2H,3H-pyrido[2,3-b][1,4]oxazin-7-yl}-5,6,7,8-tetrahydro-2,6-naphthyridin-3-yl) amino]phenyl}acetamide

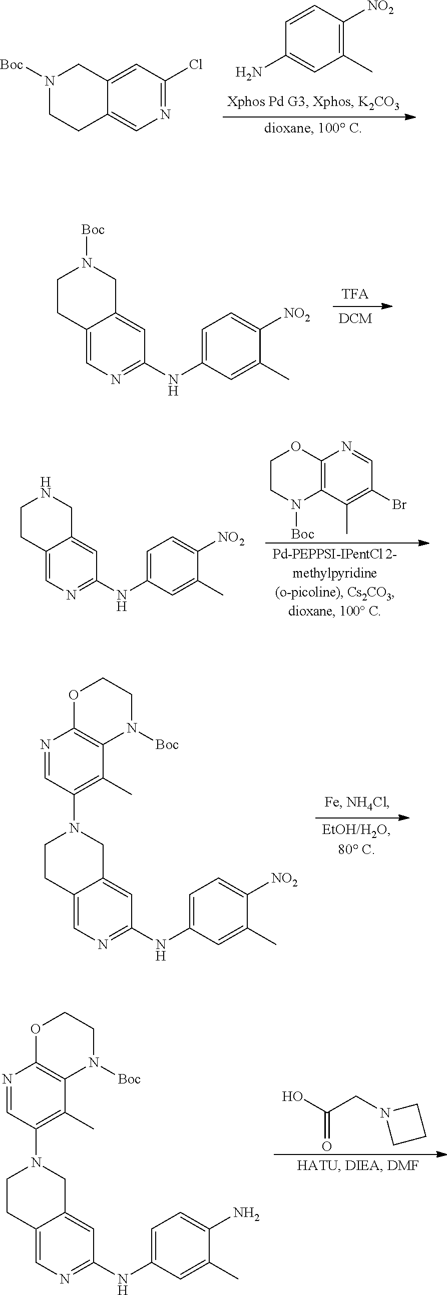

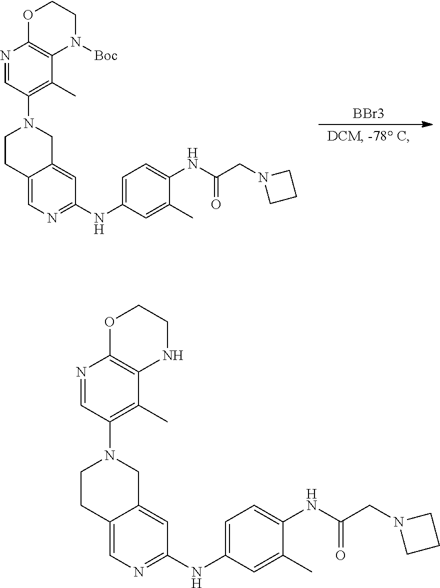 [SMILES: Cc1cc(Nc2cc3c(cn2)CCN(c2cnc4c(c2C)N(C(=O)OC(C)(C)C)CCO4)C3)ccc1NC(=O)CN1CCC1 ; "BBr3" ; "DCM, -78° C," ; Cc1cc(Nc2cc3c(cn2)CCN(c2cnc4c(c2C)NCCO4)C3)ccc1NC(=O)CN1CCC1]

tert-butyl 7-[(3-methyl-4-nitrophenyl) amino]-1,2,3,4-tetrahydro-2,6-naphthyridine-2-carboxylate: To a stirred solution of tert-butyl 7-chloro-1,2,3,4-tetrahydro-2,6-naphthyridine-2-carboxylate (800.00 mg; 2.92 mmol) and 3-methyl-4-nitroaniline (543.69 mg; 3.50 mmol) in 1,4-dioxane (8.00 mL) was added X-PHOS (292.88 mg; 0.58 mmol), Xphos Pd G₃ (260.01 mg; 0.29 mmol) and K₂CO₃ (847.27 mg; 5.84 mmol) in portion at room temperature. The resulting mixture was stirred for 2 h at 100° C. under nitrogen atmosphere. The resulting mixture was concentrated under reduced pressure. The residue was purified by silica gel column chromatography and eluted with PE/EA (1:1) to afford tert-butyl 7-[(3-methyl-4-nitrophenyl) amino]-1,2,3,4-tetrahydro-2,6-naphthyridine-2-carboxylate (580.00 mg; 51.11%) as a yellow oil.

N-(3-methyl-4-nitrophenyl)-5,6,7,8-tetrahydro-2,6-naphthyridin-3-amine: To a stirred solution of tert-butyl 7-[(3-methyl-4-nitrophenyl) amino]-1,2,3,4-tetrahydro-2,6-naphthyridine-2-carboxylate (580.00 mg; 1.49 mmol) in DCM (15.00 mL) was added TFA (3.00 mL) in portion at room temperature. After stirring for 1 h at room temperature, the mixture was basified to pH=7 with saturated NaHCO₃ (aq.). The resulting mixture was extracted with EtOAc. The combined organic layers were washed with NaCl (aq.) and dried over anhydrous Na₂SO₄. After filtration, the filtrate was concentrated under reduced pressure to afford N-(3-methyl-4-nitrophenyl)-5,6,7,8-tetrahydro-2,6-naphthyridin-3-amine (400.00 mg; Crude Product) as a yellow oil.

tert-butyl 8-methyl-7-{7-[(3-methyl-4-nitrophenyl)amino]-1,2,3,4-tetrahydro-2,6-naphthyridin-2-yl}-1H,2H,3H-pyrido[2,3-b][1,4]oxazine-1-carboxylate: To a stirred solution of N-(3-methyl-4-nitrophenyl)-5,6,7,8-tetrahydro-2,6-naphthyridin-3-amine (180.00 mg; 0.62 mmol) and tert-butyl 7-bromo-8-methyl-1H,2H,3H-pyrido[2,3-b][1,4]oxazine-1-carboxylate (213.26 mg; 0.62 mmol) in 1,4-dioxane (10.00 mL) was added Pd-PEPPSI-IPentCl 2-methylpyridine (o-picoline) (54.48 mg; 0.06 mmol) and Cs₂CO₃ (422.54 mg; 1.23 mmol) in portion at room temperature. The resulting mixture was stirred for 3 h at 100° C. under nitrogen atmosphere. The resulting mixture was concentrated under reduced pressure. The residue was purified by silica gel column chromatography and eluted with PE/EA (1:100) to afford tert-butyl 8-methyl-7-{7-[(3-methyl-4-nitrophenyl) amino]-1,2,3,4-tetrahydro-2,6-naphthyridin-2-yl}-1H,2H,3H-pyrido[2,3-b][1,4]oxazine-1-carboxylate (130.00 mg; 31.25%) as a yellow solid.

tert-butyl 7-{7-[(4-amino-3-methylphenyl) amino]-1,2,3,4-tetrahydro-2,6-naphthyridin-2-yl}-8-methyl-1H,2H,3H-pyrido[2,3-b][1,4]oxazine-1-carboxylate: To a stirred solution of tert-butyl 8-methyl-7-{7-[(3-methyl-4-nitrophenyl)amino]-1,2,3,4-tetrahydro-2,6-naphthyridin-2-yl}-1H,2H,3H-pyrido[2,3-b][1,4]oxazine-1-carboxylate (130.00 mg; 0.19 mmol) and Fe (56.74 mg; 0.96 mmol) in EtOH (6.00 mL) and H₂O (4.00 mL) was added NH₄Cl (43.36 mg; 0.77 mmol) in portion at room temperature. After stirring for 2 h at 80° C., the resulting mixture was extracted with EtOAc. The combined organic layers were washed with NaCl (aq.) and dried over anhydrous Na₂SO₄. After filtration, the filtrate was concentrated under reduced pressure to afford tert-butyl 7-{7-[(4-amino-3-methylphenyl) amino]-1,2,3,4-tetrahydro-2,6-naphthyridin-2-yl}-8-methyl-1H,2H,3H-pyrido[2,3-b][1,4]oxazine-1-carboxylate (90.00 mg; Crude Product) as a black oil.

tert-butyl 7-[7-({4-[2-(azetidin-1-yl)acetamido]-3-methylphenyl}amino)-1,2,3,4-tetrahydro-2,6-naphthyridin-2-yl]-8-methyl-1H,2H,3H-pyrido[2,3-b][1,4]oxazine-1-carboxylate: To a stirred solution of tert-butyl 7-{7-[(4-amino-3-methylphenyl)amino]-1,2,3,4-tetrahydro-2,6-naphthyridin-2-yl}-8-methyl-1H,2H,3H-pyrido[2,3-b][1,4]oxazine-1-carboxylate (90.00 mg; 0.16 mmol) and 2-(azetidin-1-yl)acetic acid hydrochloride (49.51 mg; 0.31 mmol) in DMF (10.00 mL) was added HATU (125.50 mg; 0.31 mmol) and DIEA (63.97 mg; 0.47 mmol) in portion at room temperature. The resulting mixture was stirred for 6 h at 40° C. under nitrogen atmosphere. The resulting mixture was concentrated under reduced pressure. The residue was purified by silica gel column chromatography and eluted with CH₂Cl₂/MeOH (10:1) to afford tert-butyl 7-[7-({4-[2-(azetidin-1-yl) acetamido]-3-methylphenyl}amino)-1,2,3,4-tetrahydro-2,6-naphthyridin-2-yl]-8-methyl-1H,2H,3H-pyrido[2,3-b][1,4]oxazine-1-carboxylate (80.00 mg; 81.80%) as a yellow solid.

2-(azetidin-1-yl)-N-{2-methyl-4-[(6-{8-methyl-1H,2H,3H-pyrido[2,3-b][1,4]oxazin-7-yl}-5,6,7,8-tetrahydro-2,6-naphthyridin-3-yl) amino]phenyl}acetamide: To a stirred mixture of tert-butyl 7-[7-({4-[2-(azetidin-1-yl) acetamido]-3-methylphenyl}amino)-1,2,3,4-tetrahydro-2,6-naphthyridin-2-yl]-8-methyl-1H,2H,3H-pyrido[2,3-b][1,4]oxazine-1-carboxylate (80.00 mg; 0.13 mmol) in DCM (4.00 mL) was added BBr₃ in DCM (0.40 mL; 0.40 mmol) in portion at −78° C. The resulting mixture was stirred for 1 h at −78° C. under nitrogen atmosphere. The resulting mixture was concentrated under reduced pressure. The crude product was purified by Prep-HPLC with the following conditions Column: Column: XBridge Shield RP18 OBD Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 20% B to 50% B in 9 min., 50% B; Wave Length: 254 nm; RTT (min.): 7 to afford 2-(azetidin-1-yl)-N-{2-methyl-4-[(6-{8-methyl-1H,2H,3H-pyrido[2,3-b][1,4]oxazin-7-yl}-5,6,7,8-tetrahydro-2,6-naphthyridin-3-yl) amino]phenyl}acetamide (11.40 mg; 17.70%) as a yellow solid.

HPLC: 99.4% purity, RT=2.91 min, MS: m/z=500.2[M+H]⁺. ¹H NMR (300 MHz, DMSO-d6): δ 9.03 (s, 1H), 8.73 (s, 1H), 7.99 (s, 1H), 7.40 (d, J=7.9 Hz, 2H), 7.32 (d, J=8.5 Hz, 1H), 7.27 (s, 1H), 6.57 (s, 1H), 5.50 (s, 1H), 4.19 (s, 2H), 3.98 (s, 2H), 3.45 (s, 2H), 3.34 (d, J=7.0 Hz, 4H), 3.17 (s, 2H), 3.08 (d, J=6.0 Hz, 2H), 2.82 (s, 2H), 2.16 (s, 3H), 2.13-1.98 (m, 5H).

### Example 10: Synthesis of Compound 10 N-{2-methyl-4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}-2-(pyrrolidin-1-yl)acetamide

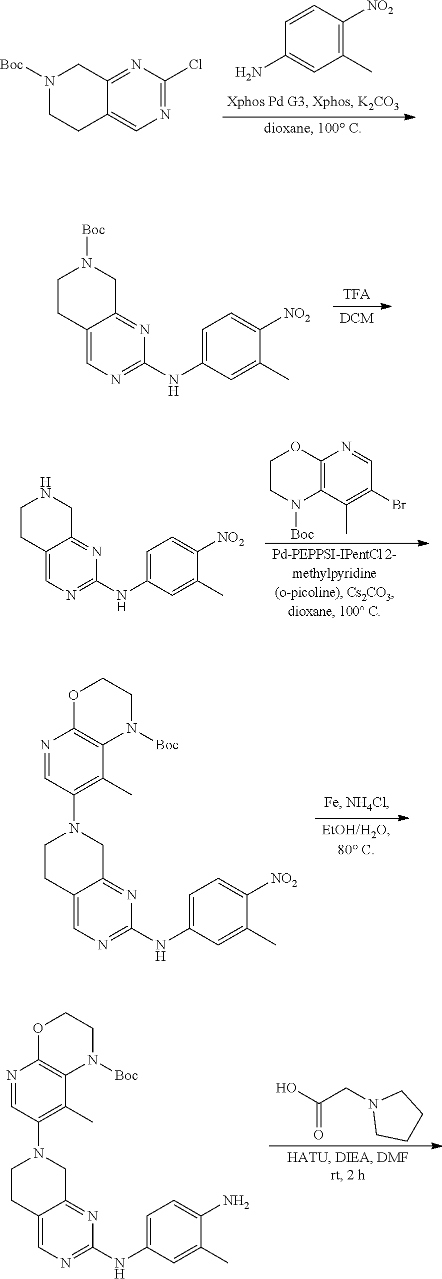

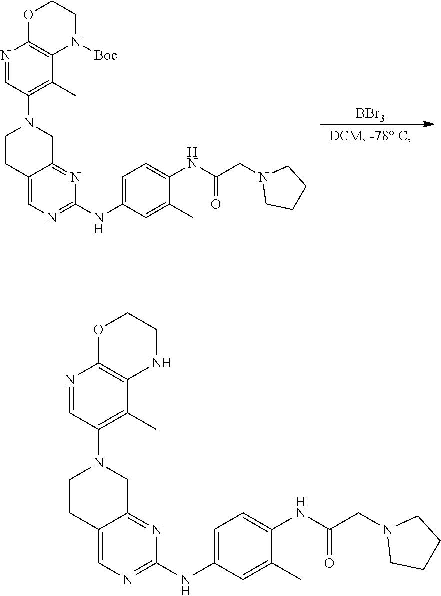 [SMILES: Cc1cc(Nc2ncc3c(n2)CN(c2cnc4c(c2C)N(C(=O)OC(C)(C)C)CCO4)CC3)ccc1NC(=O)CN1CCCC1 ; "BBr3" ; "DCM, -78° C," ; Cc1cc(Nc2ncc3c(n2)CN(c2cnc4c(c2C)NCCO4)CC3)ccc1NC(=O)CN1CCCC1]

tert-butyl 2-[(3-methyl-4-nitrophenyl) amino]-5H,6H,7H,8H-pyrido[3,4-d]pyrimidine-7-carboxylate: To a stirred mixture of tert-butyl 2-chloro-5H,6H,7H,8H-pyrido[3,4-d]pyrimidine-7-carboxylate (800.00 mg; 2.89 mmol) and 3-methyl-4-nitroaniline (590.00 mg; 3.76 mmol) in 1,4-dioxane (15.00 mL) were added X-PHOS (290.00 mg; 0.58 mmol), K₂CO₃ (1261.00 mg; 8.67 mmol) and XPhos Pd G₃ (257.00 mg; 0.29 mmol) in portions at room temperature. The resulting mixture was stirred for 2 h at 100° C. under nitrogen atmosphere. The resulting mixture was concentrated under vacuum. The residue was purified by silica gel column chromatography and eluted with PE:EA=70:30 to afford tert-butyl 2-[(3-methyl-4-nitrophenyl)amino]-5H,6H,7H,8H-pyrido[3,4-d]pyrimidine-7-carboxylate (1.00 g; 89.8%) as a yellow solid.

N-(3-methyl-4-nitrophenyl)-5H,6H,7H,8H-pyrido[3,4-d]pyrimidin-2-amine: To a stirred mixture of tert-butyl 2-[(3-methyl-4-nitrophenyl) amino]-5H,6H,7H,8H-pyrido[3,4-d]pyrimidine-7-carboxylate (980.00 mg; 2.54 mmol) in DCM (20.00 mL) was added TFA (4.00 mL) in portions at 25° C. The resulting mixture was stirred for 2 h at 25° C. under nitrogen atmosphere. The reaction was diluted with NaHCO₃ (aq.) at 25° C. The resulting mixture was extracted with EA (3×80 mL). The combined organic layers were washed with brine and dried over anhydrous MgSO₄. After filtration, the filtrate was concentrated under reduced pressure to afford N-(3-methyl-4-nitrophenyl)-5H,6H,7H,8H-pyrido[3,4-d]pyrimidin-2-amine (700.00 mg; Crude Product) as a yellow solid.

tert-butyl 8-methyl-7-{2-[(3-methyl-4-nitrophenyl)amino]-5H,6H,7H,8H-pyrido[3,4-d]pyrimidin-7-yl}-1H,2H,3H-pyrido[2,3-b][1,4]oxazine-1-carboxylate: To a stirred mixture of N-(3-methyl-4-nitrophenyl)-5H,6H,7H,8H-pyrido[3,4-d]pyrimidin-2-amine (700.00 mg; 2.45 mmol) and tert-butyl 7-bromo-8-methyl-1H,2H,3H-pyrido[2,3-b][1,4]oxazine-1-carboxylate (600.00 mg; 1.64 mmol) in 1,4-dioxane (15.00 mL) were added Cs₂CO₃ (1.90 g; 5.54 mmol) and Pd-PEPPSI-IPentCl 2-methylpyridine (o-picoline) (130.00 mg; 0.15 mmol) in portions at 25° C. The resulting mixture was stirred for 12 h at 100° C. under nitrogen atmosphere. The resulting mixture was concentrated under vacuum. The residue was purified by silica gel column chromatography and eluted with PE:EA=1:1 to afford tert-butyl 8-methyl-7-{2-[(3-methyl-4-nitrophenyl)amino]-5H,6H,7H,8H-pyrido[3,4-d]pyrimidin-7-yl}-1H,2H,3H-pyrido[2,3-b][1,4]oxazine-1-carboxylate (700.00 mg; 80.0%) as a yellow solid.

tert-butyl 7-{2-[(4-amino-3-methylphenyl) amino]-5H,6H,7H,8H-pyrido[3,4-d]pyrimidin-7-yl}-8-methyl-1H,2H,3H-pyrido[2,3-b][1,4]oxazine-1-carboxylate: To a stirred mixture of tert-butyl 8-methyl-7-{2-[(3-methyl-4-nitrophenyl)amino]-5H,6H,7H,8H-pyrido[3,4-d]pyrimidin-7-yl}-1H,2H,3H-pyrido[2,3-b][1,4]oxazine-1-carboxylate (680.00 mg; 1.27 mmol) and Fe (225.00 mg; 3.83 mmol) in EtOH (9.00 mL) were added NH₄Cl (276.00 mg; 5.11 mmol) and H₂O (6.00 mL) in portions at 25° C. The resulting mixture was stirred for 2 h at 80° C. under nitrogen atmosphere. The resulting mixture was concentrated under vacuum. The residue was purified by silica gel column chromatography and eluted with EA to afford tert-butyl 7-{2-[(4-amino-3-methylphenyl) amino]-5H,6H,7H,8H-pyrido[3,4-d]pyrimidin-7-yl}-8-methyl-1H,2H,3H-pyrido[2,3-b][1,4]oxazine-1-carboxylate (600.00 mg; 93.5%) as a yellow oil.

tert-butyl 8-methyl-7-[2-({3-methyl-4-[2-(pyrrolidin-1-yl) acetamido]phenyl}amino)-5H,6H,7H,8H-pyrido[3,4-d]pyrimidin-7-yl]-1H,2H,3H-pyrido[2,3-b][1,4]oxazine-1-carboxylate: To a stirred mixture of tert-butyl 7-{2-[(4-amino-3-methylphenyl)amino]-5H,6H,7H,8H-pyrido[3,4-d]pyrimidin-7-yl}-8-methyl-1H,2H,3H-pyrido[2,3-b][1,4]oxazine-1-carboxylate (200.00 mg; 0.32 mmol) and in DMF (8.00 mL) were added HATU (388.16 mg; 0.97 mmol) and DIEA (87.93 mg; 0.65 mmol) at room temperature. The mixture was stirred for 1 h at room temperature. The resulting mixture was quenched by ice/water and extracted with EtOAc. The combined organic layers were washed with brine and dried over anhydrous Na₂SO₄. After filtration, the filtrate was concentrated under reduced pressure. The resulting mixture was concentrated under reduced pressure and purified by silica gel column chromatography and eluted with CH₂Cl₂/MeOH (10:1) to afford tert-butyl 8-methyl-7-[2-({3-methyl-4-[2-(pyrrolidin-1-yl) acetamido]phenyl}amino)-5H,6H,7H,8H-pyrido[3,4-d]pyrimidin-7-yl]-1H,2H,3H-pyrido[2,3-b][1,4]oxazine-1-carboxylate (100.00 mg; 43.1%) as a yellow oil.

N-{2-methyl-4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}-2-(pyrrolidin-1-yl)acetamide: To a stirred mixture of tert-butyl 8-methyl-7-[2-({3-methyl-4-[2-(pyrrolidin-1-yl)acetamido]phenyl}amino)-5H,6H,7H,8H-pyrido[3,4-d]pyrimidin-7-yl]-1H,2H,3H-pyrido[2,3-b][1,4]oxazine-1-carboxylate (80.00 mg; 0.11 mmol) in DCM (4.00 mL) was added BBr₃ in DCM (0.10 mL; 0.10 mmol) dropwise at −78° C. The mixture was stirred for 1 h at −78° C. The resulting mixture was concentrated under reduced pressure. The residue was purified by Prep-HPLC (Column: Xselect CSH C18 OBD Column 30*150 mm, 5 μm, n; Mobile Phase A: water (0.1% FA), Mobile Phase B: ACN; Flow rate: 60 mL/min.; Gradient: 5% B to 27% B in 8 min, 27% B; Wave Length: 254/220 nm; RTT (min.): 6.7) to afford N-{2-methyl-4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}-2-(pyrrolidin-1-yl)acetamide (12.20 mg; 19.7%) as a yellow solid.

HPLC: 95.2% purity, RT=2.39 min. MS: m/z=515.25[M+H]⁺. ¹H NMR (300 MHz, DMSO-d6): δ 9.43 (s, 1H), 9.21 (s, 1H), 8.32 (s, 1H), 7.59 (d, J=2.4 Hz, 2H), 7.42 (d, J=8.4 Hz, 1H), 7.29 (s, 1H), 5.53 (s, 1H), 4.19 (t, J=4.2 Hz, 2H), 3.95 (s, 2H), 3.28 (d, J=17.6 Hz, 4H), 3.12 (t, J=5.6 Hz, 2H), 2.79 (d, J=5.7 Hz, 2H), 2.65 (p, J=3.6 Hz, 4H), 2.15 (s, 3H), 2.04 (s, 3H), 1.77 (p, J=3.1 Hz, 4H).

### Example 11: Synthesis of Compound 11 N-{4-[(3R)-3-(dimethylamino) pyrrolidine-1-carbonyl]phenyl}-6-{8-methyl-1H,2H,3H-pyrido[2,3-b][1,4]oxazin-7-yl}-5,6,7,8-tetrahydro-2,6-naphthyridin-3-amine

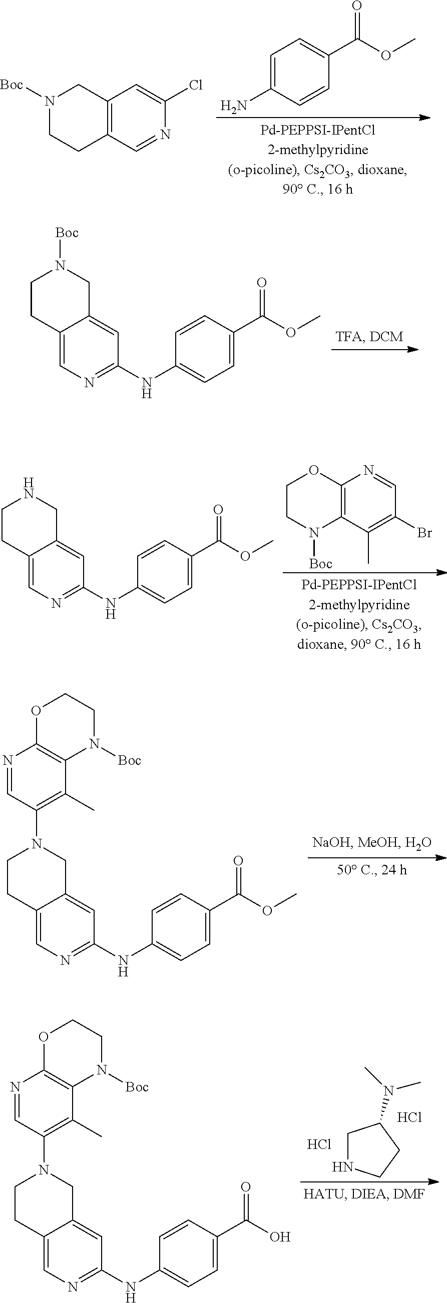

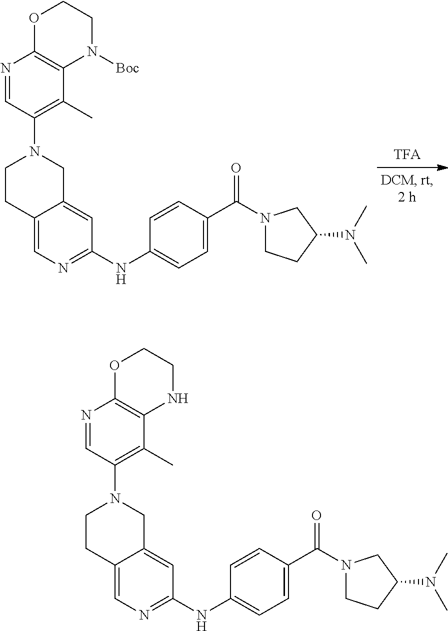 [SMILES: Cc1c(N2CCc3cnc(Nc4ccc(C(=O)N5CC[C@@H](N(C)C)C5)cc4)cc3C2)cnc2c1N(C(=O)OC(C)(C)C)CCO2 ; Cc1c(N2CCc3cnc(Nc4ccc(C(=O)N5CC[C@@H](N(C)C)C5)cc4)cc3C2)cnc2c1NCCO2]

tert-butyl 7-{[4-(methoxycarbonyl)phenyl]amino}-1,2,3,4-tetrahydro-2,6-naphthyridine-2-carboxylate: To a stirred solution of methyl 4-aminobenzoate (235.00 mg; 1.55 mmol) and tert-butyl 7-chloro-1,2,3,4-tetrahydro-2,6-naphthyridine-2-carboxylate (400.00 mg; 1.41 mmol) in 1,4-dioxane (5.00 mL) were added Cs₂CO₃ (970.00 mg; 2.83 mmol) and Pd-PEPPSI-IPentCl 2-methylpyridine (o-picoline) (125.00 mg; 0.14 mmol) in portions at room temperature under N₂ atmosphere. The resulting mixture was stirred for 1 h at 90° C. under N₂ atmosphere. The resulting mixture was diluted with water, extracted with EtOAc. The combined organic layers were washed with brine and dried over anhydrous Na₂SO₄. The resulting mixture was concentrated under reduced pressure. The residue was purified by silica gel column and eluted with 90% EtOAc in PE to afford tert-butyl 7-{[4-(methoxycarbonyl)phenyl]amino}-1,2,3,4-tetrahydro-2,6-naphthyridine-2-carboxylate (300.00 mg; 53.23%) as a yellow solid.

methyl 4-[(5,6,7,8-tetrahydro-2,6-naphthyridin-3-yl) amino]benzoate: To a stirred solution of tert-butyl 7-{[4-(methoxycarbonyl)phenyl]amino}-1,2,3,4-tetrahydro-2,6-naphthyridine-2-carboxylate (290.00 mg; 0.73 mmol) and DCM (5.00 mL) was added TFA (1.00 mL) dropwise at room temperature. The resulting mixture was stirred for 1 h at room temperature. The mixture basified with saturated NaHCO₃ (aq.) and extracted by EA. The combined organic layers were washed with brine and dried over anhydrous Na₂SO₄. After filtration, the filtrate was concentrated under reduced pressure. The residue was purified by silica gel column chromatography and eluted with EA to afford methyl 4-[(5,6,7,8-tetrahydro-2,6-naphthyridin-3-yl) amino]benzoate (90.00 mg; 96.68%) as a yellow oil.

tert-butyl 7-(7-{[4-(methoxycarbonyl)phenyl]amino}-1,2,3,4-tetrahydro-2,6-naphthyridin-2-yl)-8-methyl-1H,2H,3H-pyrido[2,3-b][1,4]oxazine-1-carboxylate: To a solution of methyl 4-[(5,6,7,8-tetrahydro-2,6-naphthyridin-3-yl)amino]benzoate (60.00 mg; 0.21 mmol) and tert-butyl 7-bromo-8-methyl-1H,2H,3H-pyrido[2,3-b][1,4]oxazine-1-carboxylate (82.00 mg; 0.23 mmol) in 1,4-dioxane (15.00 mL) were added Cs₂CO₃ (145.00 mg; 0.42 mmol) and Pd-PEPPSI-IPentCl 2-methylpyridine (o-picoline) (9.00 mg; 0.01 mmol). The resulting mixture was stirred for 3 h at 100° C. under nitrogen atmosphere. The resulting mixture was concentrated under reduced pressure. The residue was purified by silica gel column chromatography and eluted with DCM:MeOH (10:1) to afford tert-butyl 7-(7-{[4-(methoxycarbonyl)phenyl]amino}-1,2,3,4-tetrahydro-2,6-naphthyridin-2-yl)-8-methyl-1H,2H,3H-pyrido[2,3-b][1,4]oxazine-1-carboxylate (40.00 mg; 25.08%) as a yellow solid.

4-[(6-{1-[(tert-butoxy) carbonyl]-8-methyl-1H,2H,3H-pyrido[2,3-b][1,4]oxazin-7-yl}-5,6,7,8-tetrahydro-2,6-naphthyridin-3-yl)amino]benzoic acid: To a solution of tert-butyl 7-(7-{[4-(methoxycarbonyl)phenyl]amino}-1,2,3,4-tetrahydro-2,6-naphthyridin-2-yl)-8-methyl-1H,2H,3H-pyrido[2,3-b][1,4]oxazine-1-carboxylate (110.00 mg; 0.20 mmol) in MeOH (2.00 mL) and water (1.00 mL) was added sodium hydroxide (26.00 mg; 0.62 mmol). The resulting mixture was stirred for 2 h at 25° C. The mixture was basified to pH [7] with saturated NaHCO₃ (aq.). The resulting mixture was extracted with EtOAc. The combined organic layers were washed with NaCl (aq.) and dried over anhydrous Na₂SO₄. After filtration, the filtrate was concentrated under reduced pressure to afford 4-[(6-{1-[(tert-butoxy) carbonyl]-8-methyl-1H,2H,3H-pyrido[2,3-b][1,4]oxazin-7-yl}-5,6,7,8-tetrahydro-2,6-naphthyridin-3-yl)amino]benzoic acid (85.00 mg; 73.22%) as a yellow solid.

tert-butyl 7-[7-({4-[(3R)-3-(dimethylamino)pyrrolidine-1-carbonyl]phenyl}amino)-1,2,3,4-tetrahydro-2,6-naphthyridin-2-yl]-8-methyl-1H,2H,3H-pyrido[2,3-b][1,4]oxazine-1-carboxylate: To a stirred mixture of 4-[(6-{1-[(tert-butoxy)carbonyl]-8-methyl-1H,2H,3H-pyrido[2,3-b][1,4]oxazin-7-yl}-5,6,7,8-tetrahydro-2,6-naphthyridin-3-yl)amino]benzoic acid (80.00 mg; 0.14 mmol) and (3R)—N,N-dimethylpyrrolidin-3-amine dihydrochloride (43.00 mg; 0.22 mmol) in DMF (10.00 mL) was added HATU (112.00 mg; 0.28 mmol) and DIEA (0.08 mL; 0.42 mmol) at room temperature. The resulting mixture was stirred for 16 h at room temperature. The mixture was extracted with EtOAc. The combined organic layers were washed with NaCl (aq.) and dried over anhydrous Na₂SO₄. After filtration, the filtrate was concentrated under reduced pressure. The residue was purified by silica gel column chromatography and eluted with C18 to afford tert-butyl 7-[7-({4-[(3R)-3-(dimethylamino) pyrrolidine-1-carbonyl]phenyl}amino)-1,2,3,4-tetrahydro-2,6-naphthyridin-2-yl]-8-methyl-1H,2H,3H-pyrido[2,3-b][1,4]oxazine-1-carboxylate (60.00 mg; 59.01%) as a yellow solid.

N-{4-[(3R)-3-(dimethylamino) pyrrolidine-1-carbonyl]phenyl}-6-{8-methyl-1H,2H,3H-pyrido[2,3-b][1,4]oxazin-7-yl}-5,6,7,8-tetrahydro-2,6-naphthyridin-3-amine: To a stirred solution of tert-butyl 7-[7-({4-[(3R)-3-(dimethylamino)pyrrolidine-1-carbonyl]phenyl}amino)-1,2,3,4-tetrahydro-2,6-naphthyridin-2-yl]-8-methyl-1H,2H,3H-pyrido[2,3-b][1,4]oxazine-1-carboxylate (60.00 mg; 0.08 mmol) and DCM (5.00 mL) was added TFA (1.00 mL) dropwise at room temperature. The resulting mixture was stirred for 1 h at room temperature. The mixture to pH [7] with conc saturated NaHCO₃ (aq.) and extracted by EA. The combined organic layers were washed with brine and dried over anhydrous Na₂SO₄. After filtration, the filtrate was concentrated under reduced pressure. Column: XBridge Prep OBD C18 Column, 30*150 mm, 5 μm; Mobile Phase A: water (10 mmol/L NH₄HCO₃+0.1% NH₄OH), Mobile Phase B: ACN; Flow rate: 60 mL/min.; Gradient: 25% B to 55% B in 8 min., 55% B; Wave Length: 254 nm; RT1 (min.): 7) to afford N-{4-[(3R)-3-(dimethylamino)pyrrolidine-1-carbonyl]phenyl}-6-{8-methyl-1H,2H,3H-pyrido[2,3-b][1,4]oxazin-7-yl}-5,6,7,8-tetrahydro-2,6-naphthyridin-3-amine (13.10 mg; 30.71%) as a yellow solid.

HPLC: 99.7% purity, RT=4.54 min. MS: m/z=514.4 [M+H]⁺. ¹H NMR (300 MHz, DMSO-d6) δ 9.15 (s, 1H), 8.05 (s, 1H), 7.68 (d, J=8.4 Hz, 2H), 7.46 (d, J=8.1 Hz, 2H), 7.28 (s, 1H), 6.65 (s, 1H), 5.52 (s, 1H), 4.19 (s, 2H), 4.01 (s, 2H), 3.57 (m, 6H), 3.10 (s, 2H), 2.85 (s, 2H), 2.66 (s, 1H), 2.16 (d, J=18.4 Hz, 7H), 2.04 (s, 3H), 1.71 (s, 1H).

### Example 12: Synthesis of Compound 12 N-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}-2-(morpholin-4-yl)acetamide

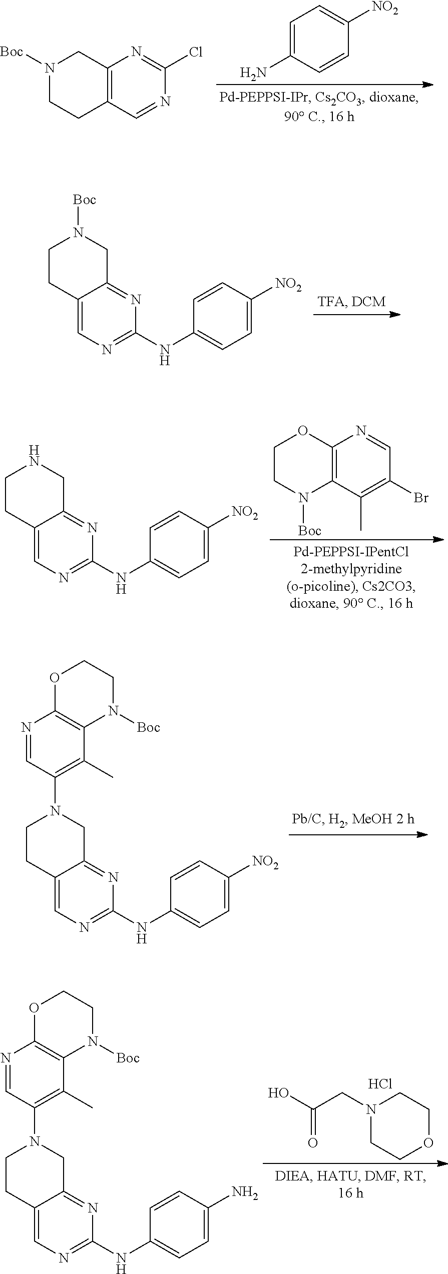

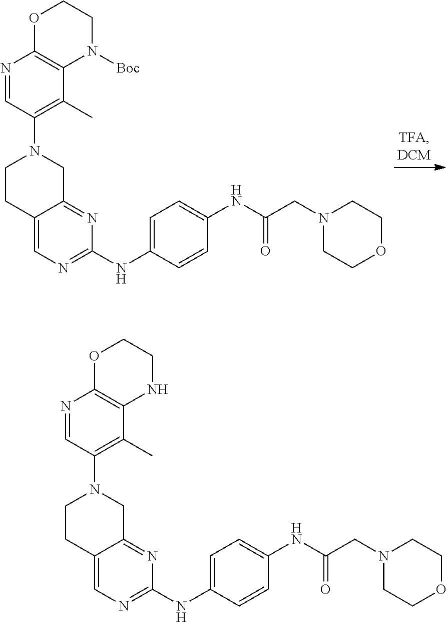 [SMILES: Cc1c(N2CCc3cnc(Nc4ccc(NC(=O)CN5CCOCC5)cc4)nc3C2)cnc2c1N(C(=O)OC(C)(C)C)CCO2 ; Cc1c(N2CCc3cnc(Nc4ccc(NC(=O)CN5CCOCC5)cc4)nc3C2)cnc2c1NCCO2]

tert-butyl 2-[(4-nitrophenyl) amino]-5H,6H,7H,8H-pyrido[3,4-d]pyrimidine-7-carboxylate: To a solution of tert-butyl 2-chloro-5H,6H,7H,8H-pyrido[3,4-d]pyrimidine-7-carboxylate (600.00 mg; 2.22 mmol) and 4-nitroaniline (383.00 mg; 2.67 mmol) in 1,4-dioxane (15.00 mL) were added Cs₂CO₃ (1526.00 mg; 4.45 mmol) and Pd-PEPPSI-IPentCl 2-methylpyridine (o-picoline) (98.00 mg, 0.11 mmol). The resulting mixture was stirred for 3 h at 100° C. under nitrogen atmosphere. The resulting mixture was concentrated under reduced pressure. The residue was purified by silica gel column chromatography and eluted with PE/EA (6:1) to afford tert-butyl 2-[(4-nitrophenyl) amino]-5H,6H,7H,8H-pyrido[3,4-d]pyrimidine-7-carboxylate (650.00 mg; 77.57%) as a yellow solid.

N-(4-nitrophenyl)-5H,6H,7H,8H-pyrido[3,4-d]pyrimidin-2-amine: To a stirred solution of tert-butyl 2-[(4-nitrophenyl) amino]-5H,6H,7H,8H-pyrido[3,4-d]pyrimidine-7-carboxylate (650.00 mg; 1.73 mmol) and DCM (5.00 mL) was added TFA (1.00 mL) dropwise at room temperature. The resulting mixture was stirred for 1 h at room temperature. The mixture was basified to pH=7 with saturated NaHCO₃ (aq.) and extracted by EA. The combined organic layers were washed with brine and dried over anhydrous Na₂SO₄. After filtration, the filtrate was concentrated under reduced pressure. The residue was purified by silica gel column chromatography and eluted with EA to afford N-(4-nitrophenyl)-5H,6H,7H,8H-pyrido[3,4-d]pyrimidin-2-amine (470.00 mg; 91.85%) as a yellow solid.

tert-butyl 8-methyl-7-{2-[(4-nitrophenyl) amino]-5H,6H,7H,8H-pyrido[3,4-d]pyrimidin-7-yl}-1H,2H,3H-pyrido[2,3-b][1,4]oxazine-1-carboxylate: To a solution of N-(4-nitrophenyl)-5H,6H,7H,8H-pyrido[3,4-d]pyrimidin-2-amine (470.00 mg; 1.69 mmol) and tert-butyl 7-bromo-8-methyl-1H,2H,3H-pyrido[2,3-b][1,4]oxazine-1-carboxylate (596.00 mg; 1.69 mmol) in 1,4-dioxane (15.00 mL) were added Cs₂CO₃ (1162.00 mg; 3.39 mmol) and Pd-PEPPSI-IPentCl 2-methylpyridine (o-picoline) (75.00 mg; 0.08 mmol). The resulting mixture was stirred for 3 h at 100° C. under nitrogen atmosphere. The resulting mixture was concentrated under reduced pressure. The residue was purified by silica gel column chromatography and eluted with DCM:MEOH (10:1) to afford tert-butyl 8-methyl-7-{2-[(4-nitrophenyl)amino]-5H,6H,7H,8H-pyrido[3,4-d]pyrimidin-7-yl}-1H,2H,3H-pyrido[2,3-b][1,4]oxazine-1-carboxylate (140.00 mg; 12.71%) as a yellow oil.

tert-butyl 7-{2-[(4-aminophenyl) amino]-5H,6H,7H,8H-pyrido[3,4-d]pyrimidin-7-yl}-8-methyl-1H,2H,3H-pyrido[2,3-b][1,4]oxazine-1-carboxylate: To a solution of tert-butyl 8-methyl-7-{2-[(4-nitrophenyl)amino]-5H,6H,7H,8H-pyrido[3,4-d]pyrimidin-7-yl}-1H,2H,3H-pyrido[2,3-b][1,4]oxazine-1-carboxylate (130.00 mg; 0.20 mmol) in MeOH (5.00 mL) was added Palladium on activated carbon (17.00 mg; 0.02 mmol) in a pressure tank. The mixture was hydrogenated at room temperature for 2 h under hydrogen atmosphere using a hydrogen balloon. The resulting mixture was filtered, and the filter cake was washed with 30 mL of EA. The filtrate was concentrated under reduced pressure and purified by C18 to afford tert-butyl 7-{2-[(4-aminophenyl) amino]-5H,6H,7H,8H-pyrido[3,4-d]pyrimidin-7-yl}-8-methyl-1H,2H,3H-pyrido[2,3-b][1,4]oxazine-1-carboxylate (60.00 mg; 53.63%) as a yellow oil.

tert-butyl 8-methyl-7-[2-({4-[2-(morpholin-4-yl)acetamido]phenyl}amino)-5H,6H,7H,8H-pyrido[3,4-d]pyrimidin-7-yl]-1H,2H,3H-pyrido[2,3-b][1,4]oxazine-1-carboxylate: To a stirred mixture of tert-butyl 7-{2-[(4-aminophenyl)amino]-5H,6H,7H,8H-pyrido[3,4-d]pyrimidin-7-yl}-8-methyl-1H,2H,3H-pyrido[2,3-b][1,4]oxazine-1-carboxylate (80.00 mg; 0.16 mmol) and 2-(morpholin-4-yl)acetic acid (49.00 mg; 0.32 mmol) in DMF (10.00 mL) was added HATU (126.44 mg; 0.32 mmol) and DIEA (0.09 mL; 0.47 mmol) at room temperature. The resulting mixture was stirred for 16 h at room temperature. The mixture was extracted with EtOAc. The combined organic layers were washed with NaCl (aq.) and dried over anhydrous Na₂SO₄. After filtration, the filtrate was concentrated under reduced pressure. The residue was purified by silica gel column chromatography and eluted with C18 to afford tert-butyl 8-methyl-7-[2-({4-[2-(morpholin-4-yl)acetamido]phenyl}amino)-5H,6H,7H,8H-pyrido[3,4-d]pyrimidin-7-yl]-1H,2H,3H-pyrido[2,3-b][1,4]oxazine-1-carboxylate (80.00 mg; 78.03%) as a yellow oil.

N-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}-2-(morpholin-4-yl)acetamide: To a stirred solution of tert-butyl 8-methyl-7-[2-({4-[2-(morpholin-4-yl)acetamido]phenyl}amino)-5H,6H,7H,8H-pyrido[3,4-d]pyrimidin-7-yl]-1H,2H,3H-pyrido[2,3-b][1,4]oxazine-1-carboxylate (75.00 mg; 0.12 mmol) and DCM (5.00 mL) was added TFA (1.00 mL) dropwise at room temperature. The resulting mixture was stirred for 1 h at room temperature. The mixture basified to pH=7 with saturated NaHCO₃ (aq.) and extracted by EA. The combined organic layers were washed with brine and dried over anhydrous Na₂SO₄. After filtration, the filtrate was concentrated under reduced pressure. The residue was purified by Prep-HPLC Column: XBridge Prep OBD C18 Column, 30*150 mm, 5 μm; Mobile Phase A: water (10 mmol/L NH₄HCO₃+0.1% NH₄OH), Mobile Phase B: ACN; Flow rate: 60 mL/min.; Gradient: 25% B to 55% B in 8 min, 55% B; Wave Length: 254 nm; RT1 (min.): 7; Number Of Runs: 2) to afford N-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}-2-(morpholin-4-yl)acetamide (9.40 mg; 15.12%) as a yellow solid.

HPLC: 96.0% purity, RT=1.97 min. MS: m/z=517.15 [M+H]⁺. ¹H NMR (300 MHz, DMSO-d6) δ 9.60 (s, 1H), 9.45 (s, 1H), 8.31 (s, 1H), 7.67 (d, J=8.0 Hz, 2H), 7.49 (d, J=8.0 Hz, 2H), 7.28 (s, 1H), 5.53 (s, 1H), 4.19 (s, 2H), 3.95 (s, 2H), 3.61 (d, J=19.3 Hz, 8H), 3.12 (d, J=9.7 Hz, 4H), 2.79 (s, 2H), 2.04 (s, 3H).

### Example 13: Synthesis of Compound 14 1,1-dimethyl-N-(7-{8-methyl-1H,2H,3H-pyrido[2,3-b][1,4]oxazin-7-yl}-5H,6H,7H,8H-pyrido[3,4-d]pyrimidin-2-yl)-2,3-dihydro-1H-isoindol-5-amine

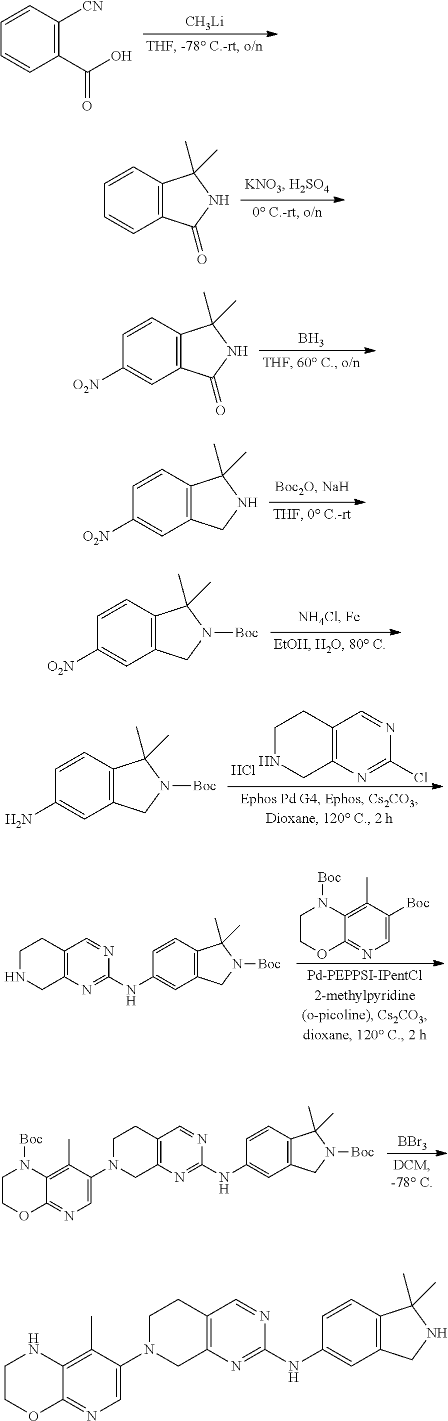

3,3-dimethyl-2,3-dihydro-1H-isoindol-1-one: To a stirred solution of 2-cyanobenzoic acid (6.00 g; 38.74 mmol) in THF (200.00 mL) was added CH₃Li (250.00 mL; 400.00 mmol) in portions at −78° C. under N₂ atmosphere. The resulting mixture was stirred for overnight at room temperature under N₂ atmosphere. The reaction was quenched with brine then extracted twice with ethyl acetate. The combined organics were washed with 1 N HCl, saturated sodium bicarbonate and water, dried over magnesium sulfate and concentrated. The residue was purified by column chromatography to give the desired product. 3,3-dimethyl-2,3-dihydro-1H-isoindol-1-one (2.80 g; 44.8%) as a yellow solid.

3,3-dimethyl-6-nitro-2,3-dihydro-1H-isoindol-1-one: To a stirred solution of 3,3-dimethyl-2,3-dihydro-1H-isoindol-1-one (2.80 g; 17.37 mmol) in sulfuric acid (42.00 mL) was added KNO₃ (2.80 mL) in portions at 0° C. The mixture was stirred overnight while gradually raising the temperature to room temperature. The reaction solution was poured into ice water, and then extracted with ethyl acetate. The extract was washed with water, saturated aqueous sodium bicarbonate solution, and brine. The organic layer was dried and evaporated to give 3,3-dimethyl-6-nitro-2,3-dihydro-1H-isoindol-1-one (3.30 g; 92.1%) as a yellow oil.

1,1-dimethyl-5-nitro-2,3-dihydro-1H-isoindole: A solution of 3,3-dimethyl-6-nitro-2,3-dihydro-1H-isoindol-1-one (3.15 g; 15.28 mmol) in BH₃ in THF (20.00 mL; 20.00 mmol) was stirred for overnight at 60° C. under nitrogen atmosphere. NaOH (aq.) and MeOH was added to the mixture and refluxed for 2 h. The resulting mixture was concentrated under vacuum. The residue was purified by silica gel column chromatography and eluted with (PE:EtOAc=7:3) to afford 1,1-dimethyl-5-nitro-2,3-dihydro-1H-isoindole (2.60 g; 83.1%) as a yellow solid.

tert-butyl 1,1-dimethyl-5-nitro-2,3-dihydro-1H-isoindole-2-carboxylate: To a solution of 1,1-dimethyl-5-nitro-2,3-dihydro-1H-isoindole (1.30 g; 6.76 mmol) in THF (40.00 mL) was added NaH (1.30 g; 32.50 mmol) at 0° C. The mixture was stirred for 1 h. (Boc)₂O (4.41 mL; 19.59 mmol) was added and the mixture was allowed to warm to room temperature and stirred for 1 h. The reaction was then quenched by adding 20 mL of water and extracted with ethyl acetate. The organic phase was concentrated under vacuum. The residue was purified by flash chromatography on silica gel eluting with petroleum ether/ethyl acetate (1/1) to afford tert-butyl 1,1-dimethyl-5-nitro-2,3-dihydro-1H-isoindole-2-carboxylate (1.80 g; 91.0%) as a yellow solid.

tert-butyl 5-amino-1,1-dimethyl-2,3-dihydro-1H-isoindole-2-carboxylate: A solution of tert-butyl 1,1-dimethyl-5-nitro-2,3-dihydro-1H-isoindole-2-carboxylate (2.00 g; 6.84 mmol) and NH₄Cl (2.40 g; 43.97 mmol) and Fe (2.40 g; 42.11 mmol) in EtOH (12.00 mL) and water (8.00 mL) was stirred for 2 h at 80° C. under nitrogen atmosphere. The resulting mixture was concentrated under vacuum. The residue was purified by silica gel column chromatography and eluted with (PE:EtOAc=7:3) to afford tert-butyl 5-amino-1,1-dimethyl-2,3-dihydro-1H-isoindole-2-carboxylate (1.00 g; 55.7%) as a dark brown oil.

tert-butyl 1,1-dimethyl-5-({5H,6H,7H,8H-pyrido[3,4-d]pyrimidin-2-yl}amino)-2,3-dihydro-1H-isoindole-2-carboxylate: A solution of tert-butyl 5-amino-1,1-dimethyl-2,3-dihydro-1H-isoindole-2-carboxylate (500.00 mg; 1.91 mmol) and 2-chloro-5H,6H,7H,8H-pyrido[3,4-d]pyrimidine hydrochloride (511.00 mg; 2.41 mmol), Cs₂CO₃ (1.85 g; 5.39 mmol), Ephos (200.00 mg; 0.36 mmol), EPhos Pd G₄ (200.00 mg; 0.21 mmol) in 1,4-dioxane (10.00 mL) was stirred for 2 h at 100° C. under nitrogen atmosphere. The resulting mixture was concentrated under vacuum. The residue was purified by silica gel column chromatography and eluted with (PE:EtOAc=7:3) to afford tert-butyl 1,1-dimethyl-5-({5H,6H,7H,8H-pyrido[3,4-d]pyrimidin-2-yl}amino)-2,3-dihydro-1H-isoindole-2-carboxylate (380.00 mg; 50.4%) as a yellow solid.

tert-butyl 5-[(7-{1-[(tert-butoxy)carbonyl]-8-methyl-1H,2H,3H-pyrido[2,3-b][1,4]oxazin-7-yl}-5H,6H,7H,8H-pyrido[3,4-d]pyrimidin-2-yl)amino]-1,1-dimethyl-2,3-dihydro-1H-isoindole-2-carboxylate: A solution of tert-butyl 1,1-dimethyl-5-({5H,6H,7H,8H-pyrido[3,4-d]pyrimidin-2-yl}amino)-2,3-dihydro-1H-isoindole-2-carboxylate (340.00 mg; 0.86 mmol) and tert-butyl 7-bromo-8-methyl-1H,2H,3H-pyrido[2,3-b][1,4]oxazine-1-carboxylate (560.00 mg; 1.58 mmol) Pd-PEPPSI-IPentCl 2-methylpyridine (o-picoline) (75.00 mg; 0.08 mmol) Cs₂CO₃ (837.00 mg; 2.44 mmol) in 1,4-dioxane (5.00 mL) was stirred for 2 h at 100° C. under nitrogen atmosphere. The resulting mixture was concentrated under vacuum. The residue was purified by silica gel column chromatography and eluted with (PE:EtOAc=7:3) to afford tert-butyl 5-[(7-{1-[(tert-butoxy) carbonyl]-8-methyl-1H,2H,3H-pyrido[2,3-b][1,4]oxazin-7-yl}-5H,6H,7H,8H-pyrido[3,4-d]pyrimidin-2-yl) amino]-1,1-dimethyl-2,3-dihydro-1H-isoindole-2-carboxylate (300.00 mg; 54.2%) as a yellow solid.

1,1-dimethyl-N-(7-{8-methyl-1H,2H,3H-pyrido[2,3-b][1,4]oxazin-7-yl}-5H,6H,7H,8H-pyrido[3,4-d]pyrimidin-2-yl)-2,3-dihydro-1H-isoindol-5-amine: To a stirred solution of tert-butyl 5-[(7-{1-[(tert-butoxy)carbonyl]-8-methyl-1H,2H,3H-pyrido[2,3-b][1,4]oxazin-7-yl}-5H,6H,7H,8H-pyrido[3,4-d]pyrimidin-2-yl)amino]-1,1-dimethyl-2,3-dihydro-1H-isoindole-2-carboxylate (270.00 mg; 0.42 mmol) in DCM (6.00 mL) was added BBr₃ in DCM (2.00 mL; 2.00 mmol) dropwise at −78° C. under nitrogen atmosphere. The resulting mixture was stirred for 2 h at −78° C. under nitrogen atmosphere. The reaction was quenched with ice water at −20° C. The resulting mixture was extracted with EtOAc. The combined organic layers were washed with brine and dried over anhydrous Na₂SO₄. After filtration, the filtrate was concentrated under reduced pressure. The crude product was purified by Prep-HPLC with the following conditions Column: XBridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 20% B to 50% B in 8 min., 50% B; Wave Length: 254 nm; RT1 (min.): 7; This resulted in 1,1-dimethyl-N-(7-{8-methyl-1H,2H,3H-pyrido[2,3-b][1,4]oxazin-7-yl}-5H,6H,7H,8H-pyrido[3,4-d]pyrimidin-2-yl)-2,3-dihydro-1H-isoindol-5-amine (37.30 mg; 19.4%) as a yellow solid.

HPLC: 97.0% purity, RT=2.44 min. MS: m/z=444.25 [M+H]⁺. ¹H NMR (400 MHz, DMSO-d6) 9.42 (s, 1H), 8.31 (s, 1H), 7.60 (s, 1H), 7.49 (d, J=8.4 Hz, 1H), 7.28 (s, 1H), 7.05 (d, J=8.2 Hz, 1H), 5.53 (s, 1H), 4.19 (t, J=4.4 Hz, 2H), 3.99 (s, 2H), 3.93 (s, 2H), 3.12 (s, 3H), 2.78 (s, 2H), 2.03 (s, 3H), 1.28 (s, 6H).

### Example 14: Synthesis of Compound 15 N1-[2-(dimethylamino)ethyl]-2-methyl-N4-(6-{8-methyl-1H,2H,3H-pyrido[2,3-b][1,4]oxazin-7-yl}-5,6,7,8-tetrahydro-2,6naphthyridin-3-yl)benzene-1,4-diamine

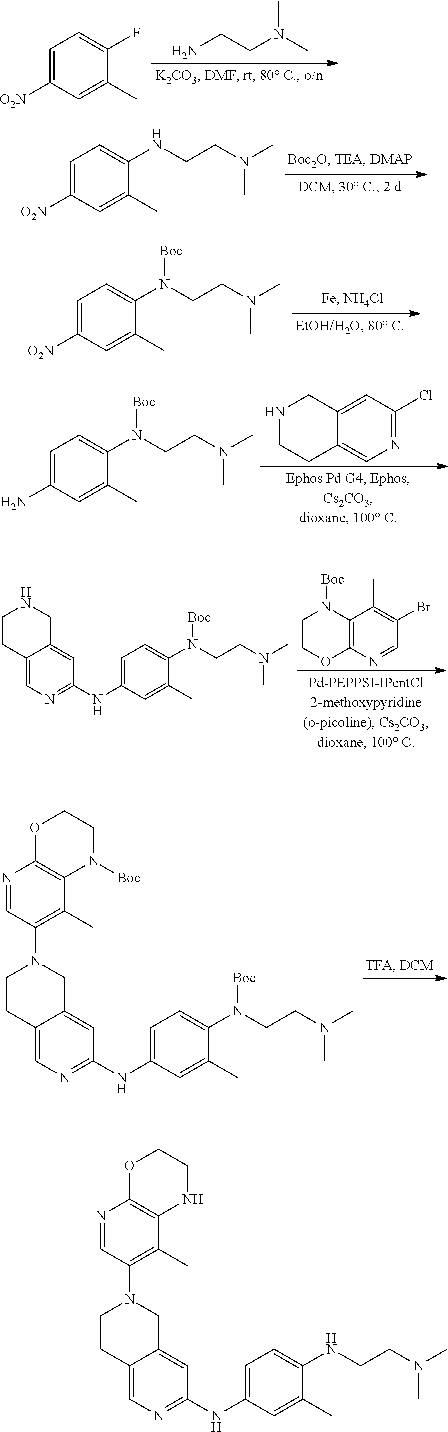

N-[2-(dimethylamino)ethyl]-2-methyl-4-nitroaniline: To a stirred mixture of 1-fluoro-2-methyl-4-nitrobenzene (1.00 g; 6.12 mmol) and (2-aminoethyl) dimethylamine (1.14 g; 12.25 mmol) in DMF (10.00 mL) was added K₂CO₃ (2.67 g; 18.37 mmol) in portions at room temperature. The resulting mixture was stirred for 5 h at 80° C. under N₂ atmosphere. The mixture was allowed to cool down to room temperature. The resulting mixture was extracted with EtOAc. The combined organic layers were washed with brine and dried over anhydrous Na₂SO₄. After filtration, the filtrate was concentrated under reduced pressure to afford N-[2-(dimethylamino) ethyl]-2-methyl-4-nitroaniline (1.30 g; Crude Product) as a yellow solid.

tert-butyl N-[2-(dimethylamino)ethyl]-N-(2-methyl-4-nitrophenyl) carbamate: To a stirred solution of N-[2-(dimethylamino)ethyl]-2-methyl-4-nitroaniline (1.26 g; 5.57 mmol) in DCM (13.00 mL) were added 4-Dimethylaminopyridine (DMAP) (0.13 g; 1.11 mmol), TEA (1.78 g; 16.71 mmol) and Boc₂O (3.68 g; 16.71 mmol) at 0° C. The resulting mixture was stirred for 2 d at 30° C. under N₂ atmosphere. The resulting mixture was extracted with EtOAc (3×20 mL). The combined organic layers were washed with brine and dried over anhydrous Na₂SO₄. After filtration, the filtrate was concentrated under reduced pressure. The residue was purified by silica gel column chromatography and eluted with DCM:MeOH (10:1) to afford tert-butyl N-[2-(dimethylamino)ethyl]-N-(2-methyl-4-nitrophenyl)carbamate (1.10 g; 48.2%) as a yellow solid.

tert-butyl N-(4-amino-2-methylphenyl)-N-[2-(dimethylamino) ethyl] carbamate: To a stirred solution of tert-butyl N-[2-(dimethylamino)ethyl]-N-(2-methyl-4-nitrophenyl)carbamate (1.00 g; 2.44 mmol) in EtOH (10.00 mL) and water (3.00 mL) were added Fe (0.72 g; 12.21 mmol) and NH₄Cl (0.82 g; 14.65 mmol) at room temperature. The resulting mixture was stirred for 1 h at 80° C. The resulting mixture was filtered, and the filter cake was washed with MeOH. The filtrate was concentrated under reduced pressure. The residue was purified by silica gel column chromatography and eluted with DCM:MeOH (10:1) to afford tert-butyl N-(4-amino-2-methylphenyl)-N-[2-(dimethylamino)ethyl]carbamate (900.00 mg; 119.7%) as a dark brown solid.

tert-butyl N-[2-(dimethylamino)ethyl]-N-{2-methyl-4-[(5,6,7,8-tetrahydro-2,6-naphthyridin-3-yl) amino]phenyl}carbamate: To a stirred mixture of tert-butyl N-(4-amino-2-methylphenyl)-N-[2-(dimethylamino)ethyl]carbamate (200.00 mg; 0.65 mmol) and 7-chloro-1,2,3,4-tetrahydro-2,6-naphthyridine (135.00 mg; 0.78 mmol) in 1,4-dioxane (2.00 mL) were added Ephos (37.00 mg; 0.07 mmol), EPhos Pd G₄ (63.00 mg; 0.07 mmol) and Cs₂CO₃ (668.00 mg; 1.95 mmol) in portions at room temperature. The resulting mixture was stirred for 1 h at 120° C. under N₂ atmosphere using a microwave reaction tube. The resulting mixture was extracted with EA. The combined organic layers were washed with brine and dried over anhydrous Na₂SO₄. After filtration, the filtrate was concentrated under reduced pressure. The residue was purified by silica gel column chromatography and eluted with DCM:MeOH (10:1) to afford tert-butyl N-[2-(dimethylamino)ethyl]-N-{2-methyl-4-[(5,6,7,8-tetrahydro-2,6-naphthyridin-3-yl)amino]phenyl}carbamate (260.00 mg; 91.3%) as a yellow solid.

tert-butyl 7-{7-[(4-{[(tert-butoxy)carbonyl][2-(dimethylamino)ethyl]amino}-3-methylphenyl)amino]-1,2,3,4-tetrahydro-2,6-naphthyridin-2-yl}-8-methyl-1H,2H,3H-pyrido[2,3-b][1,4]oxazine-1-carboxylate: To a stirred mixture of tert-butyl N-[2-(dimethylamino)ethyl]-N-{2-methyl-4-[(5,6,7,8-tetrahydro-2,6-naphthyridin-3-yl)amino]phenyl}carbamate (240.00 mg; 0.55 mmol) and tert-butyl 7-bromo-8-methyl-1H,2H,3H-pyrido[2,3-b][1,4]oxazine-1-carboxylate (219.00 mg; 0.66 mmol) in 1,4-dioxane (3.00 mL) were added Pd-PEPPSI-IPentCl 2-methylpyridine (o-picoline) (48.00 mg; 0.05 mmol) and Cs₂CO₃ (563.00 mg; 1.64 mmol) in portions at room temperature. The resulting mixture was stirred for overnight at 100° C. under N₂ atmosphere. The resulting mixture was extracted with EA. The combined organic layers were washed with brine and dried over anhydrous Na₂SO₄. The residue was purified by Prep-TLC (DCM:MeOH 30:1) to afford tert-butyl 7-{7-[(4-{[(tert-butoxy)carbonyl][2-(dimethylamino)ethyl]amino}-3-methylphenyl)amino]-1,2,3,4-tetrahydro-2,6-naphthyridin-2-yl}-8-methyl-1H,2H,3H-pyrido[2,3-b][1,4]oxazine-1-carboxylate (200.00 mg; 51.8%) as a yellow solid.

N1-[2-(dimethylamino)ethyl]-2-methyl-N4-(6-{8-methyl-1H,2H,3H-pyrido[2,3-b][1,4]oxazin-7-yl}-5,6,7,8-tetrahydro-2,6-naphthyridin-3-yl)benzene-1,4-diamine: To a stirred mixture of tert-butyl 7-{7-[(4-{[(tert-butoxy)carbonyl][2-(dimethylamino)ethyl]amino}-3-methylphenyl)amino]-1,2,3,4-tetrahydro-2,6-naphthyridin-2-yl}-8-methyl-1H,2H,3H-pyrido[2,3-b][1,4]oxazine-1-carboxylate (180.00 mg; 0.26 mmol) in DCM (3.00 mL) was added TFA (1.00 mL) dropwise at 0° C. The resulting mixture was stirred for 1 h at room temperature under N₂ atmosphere. The resulting mixture was concentrated under vacuum. The crude product was purified by Prep-HPLC with the following conditions (Column: Atlantis Prep T3 OBD Column, 19*250 mm 10 μm; Mobile Phase A: water (0.1% FA), Mobile Phase B: ACN; Flow rate: 20 mL/min.; Gradient: 20% to 50% in 8 min.; Wave Length: 254 nm; RT1 (min.): 7.53; Number Of Runs: 4) to afford N1-[2-(dimethylamino)ethyl]-2-methyl-N4-(6-{8-methyl-1H,2H,3H-pyrido[2,3-b][1,4]oxazin-7-yl}-5,6,7,8-tetrahydro-2,6-naphthyridin-3-yl) benzene-1,4-diamine (28.60 mg; 23.6%) as a yellow solid.

HPLC: 99.8% purity, RT=2.13 min. MS: m/z=474.3[M+H]⁺. ¹H NMR (400 MHz, DMSO-d6) δ 8.18 (d, J=6.9 Hz, 2H), 7.88 (s, 1H), 7.26-7.16 (m, 2H), 7.08 (d, J=2.5 Hz, 1H), 6.50 (d, J=8.6 Hz, 1H), 6.40 (s, 1H), 5.49 (d, J=3.5 Hz, 1H), 4.21-4.14 (m, 2H), 3.92 (s, 2H), 3.30 (d, J=4.7 Hz, 2H), 3.15 (t, J=6.5 Hz, 2H), 3.06 (t, J=5.7 Hz, 2H), 2.77 (t, J=5.7 Hz, 2H), 2.62 (t, J=6.4 Hz, 2H), 2.30 (s, 6H), 2.04 (d, J=15.2 Hz, 6H).

### Example 15: Synthesis of Compound 16 2-methyl-N4-(6-{8-methyl-1H,2H,3H-pyrido[2,3-b][1,4]oxazin-7-yl}-5,6,7,8-tetrahydro-2,6-naphthyridin-3-yl)-N1-[2-(morpholin-4-yl)ethyl]benzene-1,4-diamine

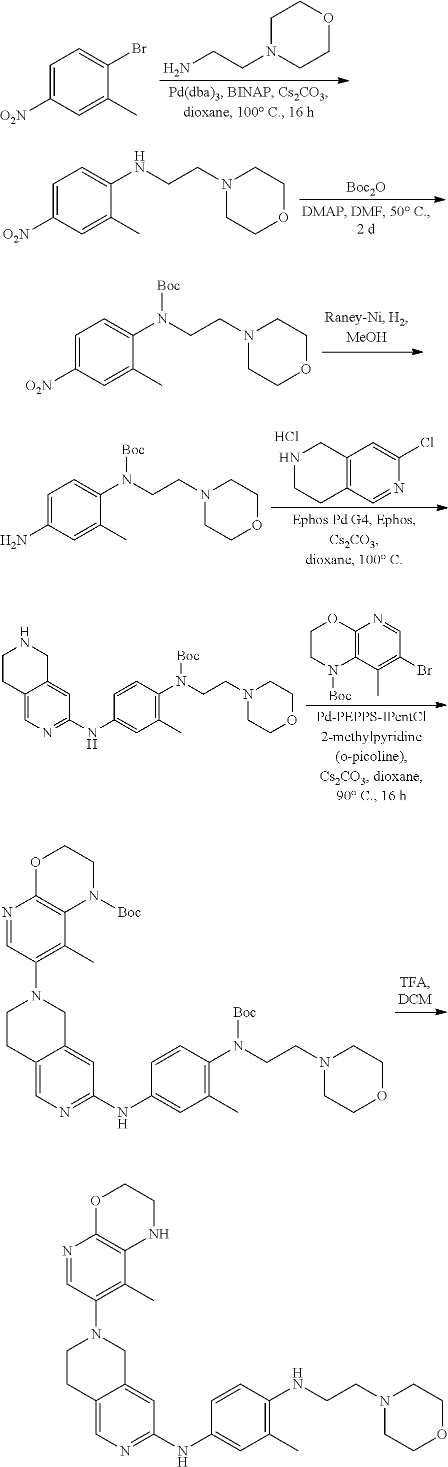

2-methyl-N-[2-(morpholin-4-yl)ethyl]-4-nitroaniline: To a stirred mixture of 2-(morpholin-4-yl)ethan-1-amine (1.86 g; 13.61 mmol) and 1-bromo-2-methyl-4-nitrobenzene (2.00 g; 9.07 mmol) in 1,4-dioxane (30.00 mL) were added Pd₂(dba)₃ (0.87 g; 0.91 mmol), BINAP (1.19 g; 1.81 mmol) and Cs₂CO₃ (6.22 g; 18.15 mmol) in portions at 25° C. The resulting mixture was stirred for 3 h at 100° C. under argon atmosphere. The resulting mixture was concentrated under reduced pressure. The residue was purified by silica gel column chromatography and eluted with PE/EA (3:1) to afford 2-methyl-N-[2-(morpholin-4-yl)ethyl]-4-nitroaniline (1.90 g; 75.46%) as a yellow solid.

tert-butyl N-(2-methyl-4-nitrophenyl)-N-[2-(morpholin-4-yl)ethyl]carbamate: To a stirred solution of 2-methyl-N-[2-(morpholin-4-yl)ethyl]-4-nitroaniline (1.30 g; 4.68 mmol) and di-tert-butyl dicarbonate (3.23 g; 14.05 mmol) in DMF (20.00 mL) were added N,N-dimethylpyridin-4-amine (0.12 g; 0.94 mmol) and TEA (2.06 mL; 14.05 mmol) in portions at room temperature under argon atmosphere. The resulting mixture was stirred for 1 d at 50° C. under argon atmosphere. The resulting mixture was concentrated under reduced pressure. The residue was purified by silica gel column and eluted with 15% EtOAc in PE to afford tert-butyl N-(2-methyl-4-nitrophenyl)-N-[2-(morpholin-4-yl)ethyl] carbamate (0.30 g; 15.80%) as a yellow solid.

tert-butyl N-(4-amino-2-methylphenyl)-N-[2-(morpholin-4-yl) ethyl] carbamate: To a solution of tert-butyl N-(2-methyl-4-nitrophenyl)-N-[2-(morpholin-4-yl)ethyl]carbamate (300.00 mg; 0.74 mmol) in MeOH (10.00 mL) was added Raney-Ni (190.30 mg; 0.22 mmol) at room temperature. The mixture stirring at room temperature for 2 h under H₂ atmosphere. The resulting mixture was filtered, and the filter cake was washed with MeOH (50 mL), the filtrate was concentrated under reduced pressure to afford tert-butyl N-(4-amino-2-methylphenyl)-N-[2-(morpholin-4-yl)ethyl] carbamate (240.00 mg; Crude Product) as a yellow oil.

tert-butyl N-{2-methyl-4-[(5,6,7,8-tetrahydro-2,6-naphthyridin-3-yl) amino]phenyl}-N-[2-(morpholin-4-yl)ethyl]carbamate: To a solution of tert-butyl N-(4-amino-2-methylphenyl)-N-[2-(morpholin-4-yl)ethyl]carbamate (230.00 mg; 0.50 mmol) and 7-chloro-1,2,3,4-tetrahydro-2,6-naphthyridine hydrochloride (157.61 mg; 0.75 mmol) in 1,4-dioxane (15.00 mL) were added Cs₂CO₃ (512.60 mg; 1.49 mmol), Ephos (56.09 mg; 0.10 mmol) and EPhos Pd G₄ (48.17 mg; 0.05 mmol). The resulting mixture was stirred for 3 h at 100° C. under nitrogen atmosphere. The mixture was concentrated under reduced pressure. The residue was purified by silica gel column chromatography and eluted with CH₂Cl₂/MeOH (11:1) to afford tert-butyl N-{2-methyl-4-[(5,6,7,8-tetrahydro-2,6-naphthyridin-3-yl) amino]phenyl}-N-[2-(morpholin-4-yl)ethyl] carbamate (180.00 mg; 76.01%) as a yellow solid.

tert-butyl 7-{7-[(4-{[(tert-butoxy)carbonyl][2-(morpholin-4-yl)ethyl]amino}-3-methylphenyl)amino]-1,2,3,4-tetrahydro-2,6-naphthyridin-2-yl}-8-methyl-1H,2H,3H-pyrido[2,3-b][1,4]oxazine-1-carboxylate: To a solution of tert-butyl N-{2-methyl-4-[(5,6,7,8-tetrahydro-2,6-naphthyridin-3-yl)amino]phenyl}-N-[2-(morpholin-4-yl)ethyl]carbamate (170.00 mg; 0.36 mmol) and tert-butyl 7-bromo-8-methyl-1H,2H,3H-pyrido[2,3-b][1,4]oxazine-1-carboxylate (178.63 mg; 0.54 mmol) in 1,4-dioxane (15.00 mL) were added Cs₂CO₃ (368.00 mg; 1.07 mmol) and Pd-PEPPSI-IPentCl 2-methylpyridine (o-picoline) (31.63 mg; 0.04 mmol). The resulting mixture was stirred for 3 h at 100° C. under nitrogen atmosphere. The mixture was concentrated under reduced pressure. The residue was purified by silica gel column chromatography and eluted with DCM/MeOH (12:1) to afford tert-butyl 7-{7-[(4-{[(tert-butoxy) carbonyl][2-(morpholin-4-yl)ethyl]amino}-3-methylphenyl)amino]-1,2,3,4-tetrahydro-2,6-naphthyridin-2-yl}-8-methyl-1H,2H,3H-pyrido[2,3-b][1,4]oxazine-1-carboxylate (160.00 mg; 56.46%) as a yellow oil.

2-methyl-N4-(6-{8-methyl-1H,2H,3H-pyrido[2,3-b][1,4]oxazin-7-yl}-5,6,7,8-tetrahydro-2,6-naphthyridin-3-yl)-N1-[2-(morpholin-4-yl) ethyl]benzene-1,4-diamine: To a stirred mixture of tert-butyl 7-{7-[(4-{[(tert-butoxy)carbonyl][2-(morpholin-4-yl)ethyl]amino}-3-methylphenyl)amino]-1,2,3,4-tetrahydro-2,6-naphthyridin-2-yl}-8-methyl-1H,2H,3H-pyrido[2,3-b][1,4]oxazine-1-carboxylate (150.00 mg; 0.19 mmol) in DCM (5.00 mL) and was added TFA (1.00 mL) at room temperature. After stirring for 3 h at room temperature, the mixture was concentrated under reduced pressure. The crude product was purified by Prep-HPLC (Column: XBridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 25% B to 55% B in 8 min., 55% B; Wave Length: 254 nm; RT1 (min.): 7) to afford 2-methyl-N4-(6-{8-methyl-1H,2H,3H-pyrido[2,3-b][1,4]oxazin-7-yl}-5,6,7,8-tetrahydro-2,6-naphthyridin-3-yl)-N1-[2-(morpholin-4-yl)ethyl]benzene-1,4-diamine (34.20 mg; 34.67%) as a yellow solid.

HPLC: 98.9% purity, RT=3.58 min. MS: m/z=516.35[M+H]⁺. ¹H NMR (400 MHz, DMSO-d6): δ 8.19 (s, 1H), 7.88 (s, 1H), 7.24 (s, 1H), 7.18 (dd, J=8.5, 2.6 Hz, 1H), 7.08 (d, J=2.5 Hz, 1H), 6.50 (d, J=8.6 Hz, 1H), 6.40 (s, 1H), 5.50 (t, J=2.7 Hz, 1H), 4.43 (s, 1H), 4.18 (dd, J=5.1, 3.5 Hz, 2H), 3.92 (s, 2H), 3.59 (q, J=4.8 Hz, 4H), 3.29 (dt, J=6.8, 3.3 Hz, 2H), 3.13 (s, 2H), 3.05 (t, J=5.4 Hz, 2H), 2.77 (t, J=5.6 Hz, 2H), 2.56 (t, J=6.5 Hz, 2H), 2.41 (s, 4H), 2.10-1.99 (m, 6H).

### Example 16: Synthesis of Compound 13 2-(dimethylamino)-N-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}acetamide

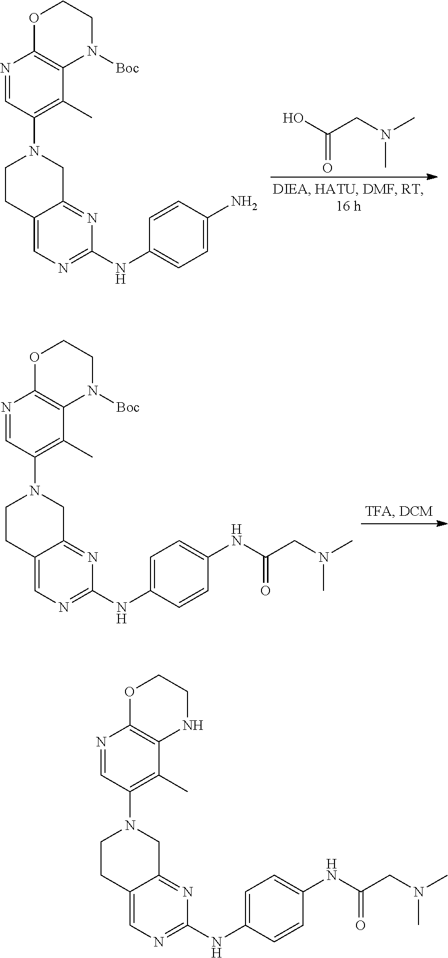

tert-butyl 7-[2-({4-[2-(dimethylamino)acetamido]phenyl}amino)-5H,6H,7H,8H-pyrido[3,4-d]pyrimidin-7-yl]-8-methyl-1H,2H,3H-pyrido[2,3-b][1,4]oxazine-1-carboxylate: To a stirred mixture of tert-butyl 7-{2-[(4-aminophenyl)amino]-5H,6H,7H,8H-pyrido[3,4-d]pyrimidin-7-yl}-8-methyl-1H,2H,3H-pyrido[2,3-b][1,4]oxazine-1-carboxylate (60.00 mg; 0.11 mmol) and 2-(dimethylamino)acetic acid (23.00 mg; 0.21 mmol) in DMF (10.00 mL) were added HATU (85.84 mg; 0.21 mmol) and DIEA (0.06 mL; 0.32 mmol) at room temperature. The resulting mixture was stirred for 16 h at room temperature. The resulting mixture was extracted with EtOAc. The combined organic layers were washed with NaCl (aq.) and dried over anhydrous Na₂SO₄. After filtration, the filtrate was concentrated under reduced pressure. The residue was purified by silica gel column chromatography and eluted with DCM/MeOH (6:1) to afford tert-butyl 7-[2-({4-[2-(dimethylamino) acetamido]phenyl}amino)-5H,6H,7H,8H-pyrido[3,4-d]pyrimidin-7-yl]-8-methyl-1H,2H,3H-pyrido[2,3-b][1,4]oxazine-1-carboxylate (70.00 mg; 98.04%) as a yellow solid.

2-(dimethylamino)-N-{4-[(7-{8-methyl-1H,2H,3H-pyrido[2,3-b]oxazin-7-yl}-5H,6H,7H,8H-pyrido[3,4-d]pyrimidin-2-yl) amino]phenyl}acetamide: To a stirred solution of tert-butyl 7-[2-({4-[2-(dimethylamino) acetamido]phenyl}amino)-5H,6H,7H,8H-pyrido[3,4-d]pyrimidin-7-yl]-8-methyl-1H,2H,3H-pyrido[2,3-b][1,4]oxazine-1-carboxylate (80.00 mg; 0.12 mmol) and DCM (5.00 mL) was added TFA (1.00 mL) dropwise at room temperature. The resulting mixture was stirred for 1 h at room temperature. The mixture was basified pH=7 with saturated NaHCO₃ (aq.) and extracted by EA. The combined organic layers were washed with brine and dried over anhydrous Na₂SO₄. After filtration, the filtrate was concentrated under reduced pressure (Column: XBridge Prep OBD C18 Column, 30*150 mm, 5 μm; Mobile Phase A: water (10 mmol/L NH₄HCO₃+0.1% NH₄OH), Mobile Phase B: ACN; Flow rate: 60 mL/min.; Gradient: 25% B to 55% B in 8 min, 55% B; Wave Length: 254 nm; RTT (min.): 7) to afford 2-(dimethylamino)-N-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}acetamide (7.80 mg; 13.14%) as a yellow solid.

HPLC: 96.0% purity, RT=2.23 min. MS: m/z=475.15[M+H]⁺. ¹H NMR (300 MHz, DMSO-d6) δ 9.60 (s, 1H), 9.44 (s, 1H), 8.23 (d, J=49.4 Hz, 1H), 7.66 (d, J=9.0 Hz, 2H), 7.52 (d, J=9.0 Hz, 2H), 7.29 (s, 1H), 5.52 (d, J=3.3 Hz, 1H), 4.34-4.11 (m, 2H), 3.95 (s, 2H), 3.11 (d, J=6.9 Hz, 6H), 2.79 (t, J=5.5 Hz, 2H), 2.31 (s, 6H), 2.04 (s, 3H).

### Example 17: Synthesis of Compound 17 N1,2-dimethyl-N4-(7-{8-methyl-1H,2H,3H-pyrido[2,3-b][1,4]oxazin-7-yl}-5H,6H,7H,8H-pyrido[3,4-d]pyrimidin-2-yl)-N1-[2-(morpholin-4-yl)ethyl]benzene-1,4-diamine; formic acid

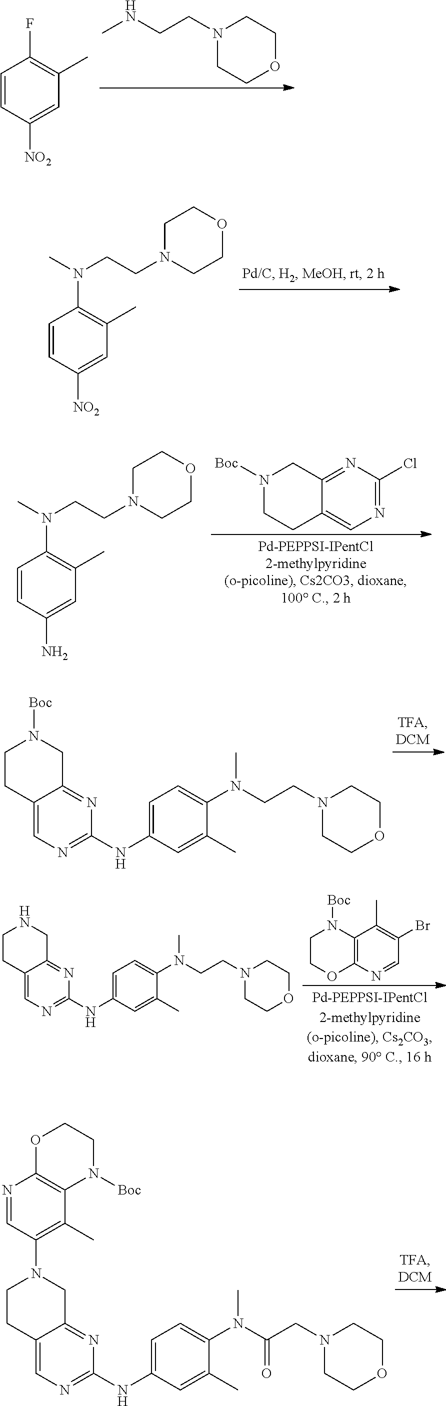

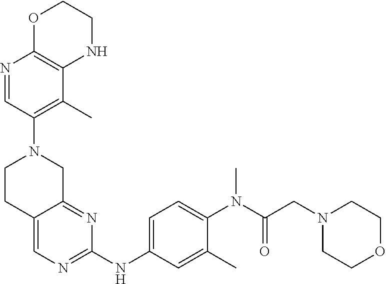 [SMILES: Cc1cc(Nc2ncc3c(n2)CN(c2cnc4c(c2C)NCCO4)CC3)ccc1N(C)C(=O)CN1CCOCC1]

N,2-dimethyl-N-[2-(morpholin-4-yl)ethyl]-4-nitroaniline: To a stirred mixture of 1-fluoro-2-methyl-4-nitrobenzene (384.00 mg; 2.01 mmol) and methyl[2-(morpholin-4-yl)ethyl]amine (319.65 mg; 2.22 mmol) in ACN (6.00 mL) was added K₂CO₃ (586.28 mg; 4.03 mmol) at room temperature. The mixture was stirred for 16 h at 80 degrees C. The resulting mixture was concentrated under reduced pressure and purified by silica gel column chromatography and eluted with PE/EtOAc (1:1) to afford N,2-dimethyl-N-[2-(morpholin-4-yl)ethyl]-4-nitroaniline (240.00 mg; 41.2%) as a yellow oil.

N1,2-dimethyl-N1-[2-(morpholin-4-yl)ethyl]benzene-1,4-diamine: To a solution of N,2-dimethyl-N-[2-(morpholin-4-yl)ethyl]-4-nitroaniline (230.00 mg; 0.81 mmol) in MeOH (4.00 mL) mL was added Raney-Ni (40.00 mg; 0.44 mmol) at room temperature. After stirring for 2 h at room temperature under hydrogen atmosphere, the resulting mixture was filtered. The filter cake was washed with MeOH. The crude product was used in the next step directly without further purification. This resulted in N1,2-dimethyl-N1-[2-(morpholin-4-yl)ethyl]benzene-1,4-diamine (190.00 mg; 69.8%) as a brown oil.

tert-butyl 2-[(3-methyl-4-{methyl[2-(morpholin-4-yl)ethyl]amino}phenyl)amino]-5H,6H,7H,8H-pyrido[3,4-d]pyrimidine-7-carboxylate: To a stirred mixture of tert-butyl 2-chloro-5H,6H,7H,8H-pyrido[3,4-d]pyrimidine-7-carboxylate (582.05 mg; 1.76 mmol) and N1,2-dimethyl-N1-[2-(morpholin-4-yl)ethyl]benzene-1,4-diamine (292.00 mg; 1.17 mmol; 1.00 eq.) in 1,4-dioxane (13.00 mL) were added Pd-PEPPSI-IPentCl 2-methylpyridine (o-picoline) (103.56 mg; 0.12 mmol) and Cs₂CO₃ (803.26 mg; 2.34 mmol) at room temperature. The mixture was stirred for 2 h at 100° C. The resulting mixture was concentrated under reduced pressure, purified by silica gel column chromatography and eluted with PE/EtOAc (1:1) to afford tert-butyl 2-[(3-methyl-4-{methyl[2-(morpholin-4-yl)ethyl]amino}phenyl)amino]-5H,6H,7H,8H-pyrido[3,4-d]pyrimidine-7-carboxylate (320.00 mg; 50.5%) as a yellow oil.

N1,2-dimethyl-N1-[2-(morpholin-4-yl)ethyl]-N4-{5H,6H,7H,8H-pyrido[3,4-d]pyrimidin-2-yl}benzene-1,4-diamine: To a stirred mixture of tert-butyl 2-[(3-methyl-4-{methyl[2-(morpholin-4-yl)ethyl]amino}phenyl)amino]-5H,6H,7H,8H-pyrido[3,4-d]pyrimidine-7-carboxylate (530.00 mg; 0.97 mmol) and TFA (3.00 mL) in DCM (15.00 mL) at room temperature. The mixture was stirred for 1 h at room temperature. The mixture was basified to pH=9 with sat. NaHCO₃ (aq.) and extracted with EtOAc. The combined organic layers were washed with brine and dried over anhydrous Na₂SO₄. After filtration, the filtrate was concentrated under reduced pressure. This resulted in N1,2-dimethyl-N1-[2-(morpholin-4-yl)ethyl]-N4-{5H,6H,7H,8H-pyrido[3,4-d]pyrimidin-2-yl}benzene-1,4-diamine (230.00 mg; 59.7%) as a yellow oil.

tert-butyl 8-methyl-7-{2-[(3-methyl-4-{methyl[2-(morpholin-4-yl)ethyl]amino}phenyl)amino]-5H,6H,7H,8H-pyrido[3,4-d]pyrimidin-7-yl}-1H,2H,3H-pyrido[2,3-b][1,4]oxazine-1-carboxylate: To a stirred mixture of N1,2-dimethyl-N1-[2-(morpholin-4-yl)ethyl]-N4-{5H,6H,7H,8H-pyrido[3,4-d]pyrimidin-2-yl}benzene-1,4-diamine (410.00 mg; 0.87 mmol) and tert-butyl 7-bromo-8-methyl-1H,2H,3H-pyrido[2,3-b][1,4]oxazine-1-carboxylate (574.45 mg; 1.75 mmol) in 1,4-dioxane (10.00 mL) were added Pd-PEPPSI-IPentCl 2-methylpyridine (o-picoline) (77.16 mg; 0.09 mmol) and Cs₂CO₃ (598.49 mg; 1.75 mmol) at room temperature. The mixture was stirred for 2 h at 100° C. The resulting mixture was concentrated under reduced pressure and purified by silica gel column chromatography and eluted with PE/EtOAc (1:1) to afford tert-butyl 8-methyl-7-{2-[(3-methyl-4-{methyl[2-(morpholin-4-yl)ethyl]amino}phenyl)amino]-5H,6H,7H,8H-pyrido[3,4-d]pyrimidin-7-yl}-1H,2H,3H-pyrido[2,3-b][1,4]oxazine-1-carboxylate (160.00 mg; 23.8%) as a yellow oil.

N1,2-dimethyl-N4-(7-{8-methyl-1H,2H,3H-pyrido[2,3-b][1,4]oxazin-7-yl}-5H,6H,7H,8H-pyrido[3,4-d]pyrimidin-2-yl)-N1-[2-(morpholin-4-yl)ethyl]benzene-1,4-diamine; formic acid: To a stirred mixture of tert-butyl 8-methyl-7-{2-[(3-methyl-4-{methyl[2-(morpholin-4-yl)ethyl]amino}phenyl)amino]-5H,6H,7H,8H-pyrido[3,4-d]pyrimidin-7-yl}-1H,2H,3H-pyrido[2,3-b][1,4]oxazine-1-carboxylate (150.00 mg) and TFA (1.00 mL; 12.79 mmol) in DCM (5.00 mL) at room temperature. The mixture was stirred for 1 h at room temperature. The resulting mixture was concentrated under reduced pressure. The residue was purified by Prep-HPLC (Column: X select CSH C18 OBD Column 30*150 mm, 5 μm, n; Mobile Phase A: water (0.1% FA), Mobile Phase B: ACN; Flow rate: 60 mL/min.; Gradient: 5% B to 27% B in 8 min., 27% B; Wavelength: 254/220 nm; RT1 (min.): 6.7; Number Of Runs: 3) to yield N1,2-dimethyl-N4-(7-{8-methyl-1H,2H,3H-pyrido[2,3-b][1,4]oxazin-7-yl}-5H,6H,7H,8H-pyrido[3,4-d]pyrimidin-2-yl)-N1-[2-(morpholin-4-yl)ethyl]benzene-1,4-diamine; formic acid (47.20 mg; 52.1%) as a yellow solid.

HPLC: 97.9% purity, RT=2.83 min. MS: m/z=531.6 [M+H]⁺. ¹H NMR (300 MHz, DMSO-d6): δ 9.46 (s, 1H), 9.35 (s, 1H), 8.29 (s, 1H), 7.62-7.53 (m, 1H), 7.49 (d, J=2.5 Hz, 1H), 7.27 (s, 1H), 7.05 (d, J=8.7 Hz, 1H), 4.19 (d, J=1.3 Hz, 2H), 3.93 (s, 2H), 3.26 (s, 4H), 3.30 (s, 10H), 3.10 (s, 2H), 2.77 (s, 2H), 2.54 (d, J=9.9 Hz, 3H), 2.22 (s, 3H), 2.03 (s, 3H).

### Example 18: Synthesis of Compound 18 6-(1-methanesulfonylcyclopropyl)-N-(7-{8-methyl-1H,2H,3H-pyrido[2,3-b][1,4]oxazin-7-yl}-5H,6H,7H,8H-pyrido[3,4-d]pyrimidin-2-yl)pyridin-3-amine

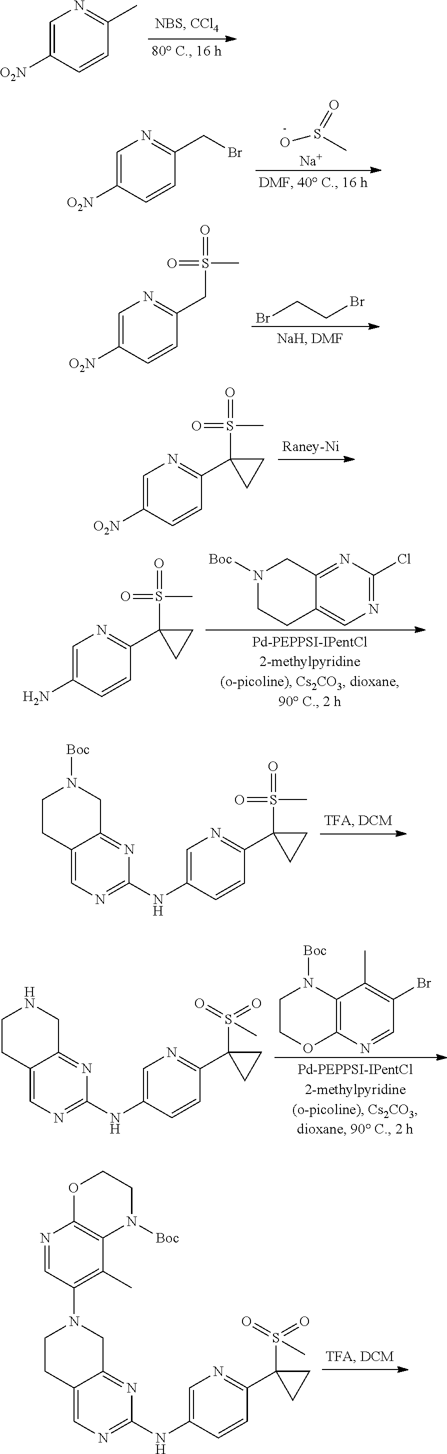

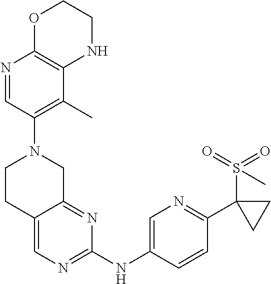 [SMILES: Cc1c(N2CCc3cnc(Nc4ccc(C5(S(C)(=O)=O)CC5)nc4)nc3C2)cnc2c1NCCO2]

2-(bromomethyl)-5-nitropyridine: To a stirred solution of 2-methyl-5-nitropyridine (10.00 g; 70.95 mmol) in CCl₄ (100.00 mL) was added NBS (38.66 g; 212.85 mmol) and AIBN (4.76 g; 28.38 mmol) in portions at room temperature. After stirring for 16 h at 80° C., the resulting mixture was concentrated under reduced pressure. The residue was purified by silica gel column chromatography and eluted with PE/EA (6:1) to afford 2-(bromomethyl)-5-nitropyridine (6.20 g; 38.58%) as a yellow solid.

2-(methanesulfonylmethyl)-5-nitropyridine: To a stirred solution of 2-(bromomethyl)-5-nitropyridine (3.00 g; 13.24 mmol) in DMF (7.00 mL) was added sodium methanesulfinate (2.73 g; 26.49 mmol) at room temperature. The resulting mixture was stirred for 2 h at 65° C. under nitrogen atmosphere. The resulting mixture was concentrated under reduced pressure. The residue was purified by silica gel column chromatography and eluted with PE/EA (1:1) to afford 2-(methanesulfonylmethyl)-5-nitropyridine (1.20 g; 41.13%) as a yellow solid.

2-(1-methanesulfonylcyclopropyl)-5-nitropyridine: To a solution of 2-(methanesulfonylmethyl)-5-nitropyridine (696.00 mg; 3.16 mmol) in DMF (10.00 mL) was added NaH (400 mg; 15.80 mmol; 60% purity) and 1,2-dibromoethane (2499.11 mg; 12.64 mmol) at 0 degrees C. After stirring for 4 h at 30° C., the resulting mixture was concentrated under reduced pressure. The residue was purified by silica gel column chromatography and eluted with PE/EtOAc (3:1) to afford 2-(1-methanesulfonylcyclopropyl)-5-nitropyridine (155.00 mg; 19.77%) as a yellow oil.

6-(1-methanesulfonylcyclopropyl)pyridin-3-amine: To a solution of 2-(1-methanesulfonylcyclopropyl)-5-nitropyridine (323.00 mg; 1.30 mmol) in MeOH (20.00 mL) was added Raney-Ni (134.99 mg; 1.50 mmol) at room temperature. After stirring for 1 h at room temperature under hydrogen atmosphere, the resulting mixture was filtered. The filter cake was washed with MeOH and then filtrate was concentrated under reduced pressure to afford 6-(1-methanesulfonylcyclopropyl)pyridin-3-amine (273.00 mg; 97.10%) as a brown oil.

tert-butyl 2-{[6-(1-methanesulfonylcyclopropyl)pyridin-3-yl]amino}-5H,6H,7H,8H-pyrido[3,4-d]pyrimidine-7-carboxylate: To a stirred solution of 6-(1-methanesulfonylcyclopropyl)pyridin-3-amine (263.00 mg; 1.22 mmol) and tert-butyl 2-chloro-5H,6H,7H,8H-pyrido[3,4-d]pyrimidine-7-carboxylate (332.13 mg; 1.22 mmol) in 1,4-dioxane (10.00 mL) was added Pd-PEPPSI-IPentCl 2-methylpyridine (o-picoline) (107.67 mg; 0.12 mmol) and Cs₂CO₃ (835.08 mg; 2.43 mmol). The resulting mixture was stirred for 3 h at 100° C. under nitrogen atmosphere. The resulting mixture was concentrated under reduced pressure. The residue was purified by silica gel column chromatography and eluted with PE/EtOAc (1:100) to afford tert-butyl 2-{[6-(1-methanesulfonylcyclopropyl)pyridin-3-yl]amino}-5H,6H,7H,8H-pyrido[3,4-d]pyrimidine-7-carboxylate (263.00 mg; 47.62%) as a yellow oil.

6-(1-methanesulfonylcyclopropyl)-N-{5H,6H,7H,8H-pyrido[3,4-d]pyrimidin-2-yl}pyridin-3-amine: To a stirred solution of tert-butyl 2-{[6-(1-methanesulfonylcyclopropyl)pyridin-3-yl]amino}-5H,6H,7H,8H-pyrido[3,4-d]pyrimidine-7-carboxylate (253.00 mg; 0.56 mmol) in DCM (10.00 mL) was added TFA (2.00 mL) in portions at room temperature. After stirring for 1 h at room temperature, the resulting mixture was concentrated under reduced pressure to afford 6-(1-methanesulfonylcyclopropyl)-N-{5H,6H,7H,8H-pyrido[3,4-d]pyrimidin-2-yl}pyridin-3-amine (130.00 mg; 66.16%) as a brown oil.

tert-butyl 7-(2-{[6-(1-methanesulfonylcyclopropyl)pyridin-3-yl]amino}-5H,6H,7H,8H-pyrido[3,4-d]pyrimidin-7-yl)-8-methyl-1H,2H,3H-pyrido[2,3-b][1,4]oxazine-1-carboxylate: To a stirred solution of 6-(1-methanesulfonylcyclopropyl)-N-{5H,6H,7H,8H-pyrido[3,4-d]pyrimidin-2-yl}pyridin-3-amine (130.00 mg; 0.37 mmol) and tert-butyl 7-bromo-8-methyl-1H,2H,3H-pyrido[2,3-b][1,4]oxazine-1-carboxylate (127.72 mg; 0.37 mmol) in 1,4-dioxane (10.00 mL) was added Pd-PEPPSI-IPentCl 2-methylpyridine (o-picoline) (32.63 mg; 0.04 mmol) and Cs₂CO₃ (253.07 mg; 0.74 mmol). The resulting mixture was stirred for 3 h at 100° C. under nitrogen atmosphere. The resulting mixture was concentrated under reduced pressure. The residue was purified by silica gel column chromatography and eluted with CH₂Cl₂/MeOH (10:1) to afford tert-butyl 7-(2-{[6-(1-methanesulfonylcyclopropyl)pyridin-3-yl]amino}-5H,6H,7H,8H-pyrido[3,4-d]pyrimidin-7-yl)-8-methyl-1H,2H,3H-pyrido[2,3-b][1,4]oxazine-1-carboxylate (196.00 mg; 75.05%) as a yellow oil.

6-(1-methanesulfonylcyclopropyl)-N-(7-{8-methyl-1H,2H,3H-pyrido[2,3-b][1,4]oxazin-7-yl}-5H,6H,7H,8H-pyrido[3,4-d]pyrimidin-2-yl)pyridin-3-amine: To a stirred solution of tert-butyl 7-(2-{[6-(1-methanesulfonylcyclopropyl)pyridin-3-yl]amino}-5H,6H,7H,8H-pyrido[3,4-d]pyrimidin-7-yl)-8-methyl-1H,2H,3H-pyrido[2,3-b][1,4]oxazine-1-carboxylate (196.00 mg; 0.28 mmol) in DCM (10.00 mL) was added TFA (2.00 mL) in portions at room temperature. After stirring for 1 h at room temperature, the resulting mixture was concentrated under reduced pressure. The crude product was purified by Prep-HPLC with the following conditions: Column: XBridge Shield RP18 OBD Column, 30*150 mm, 5 μm; Mobile Phase A: water (10 mmol/L NH₄HCO₃+0.05% NH4OH), Mobile Phase B: ACN; Flow rate: 60 mL/min.; Gradient: 18% B to 48% B in 9 min., 48% B; Wave Length: 254 nm; RT1 (min.): 7; Number Of Runs: 2, to afford 6-(1-methanesulfonylcyclopropyl)-N-(7-{8-methyl-1H,2H,3H-pyrido[2,3-b][1,4]oxazin-7-yl}-5H,6H,7H,8H-pyrido[3,4-d]pyrimidin-2-yl)pyridin-3-amine (32.90 mg; 23.90%) as a yellow solid.

HPLC: 99.2% purity, RT=2.79 min. MS: m/z=494.2.6 [M+H]⁺. ¹H NMR (300 MHz, DMSO-d6): δ 9.90 (s, 1H), 8.91 (d, J=2.6 Hz, 1H), 8.40 (s, 1H), 8.25 (dd, J=8.6, 2.7 Hz, 1H), 7.51 (d, J=8.6 Hz, 1H), 7.29 (s, 1H), 5.54 (s, 1H), 4.19 (d, J=4.9 Hz, 2H), 4.00 (s, 2H), 3.33 (s, 2H), 3.13 (s, 2H), 3.01 (s, 3H), 2.82 (s, 2H), 2.04 (s, 3H), 1.63 (q, J=4.8, 4.3 Hz, 2H), 1.38 (t, J=3.7 Hz, 2H).

### Example 19: Synthesis of Compound 19 5-fluoro-6-(methanesulfonylmethyl)-N-(7-{8-methyl-1H,2H,3H-pyrido[2,3-b][1,4]oxazin-7-yl}-5H,6H,7H,8H-pyrido[3,4-d]pyrimidin-2-yl)pyridin-3-amine

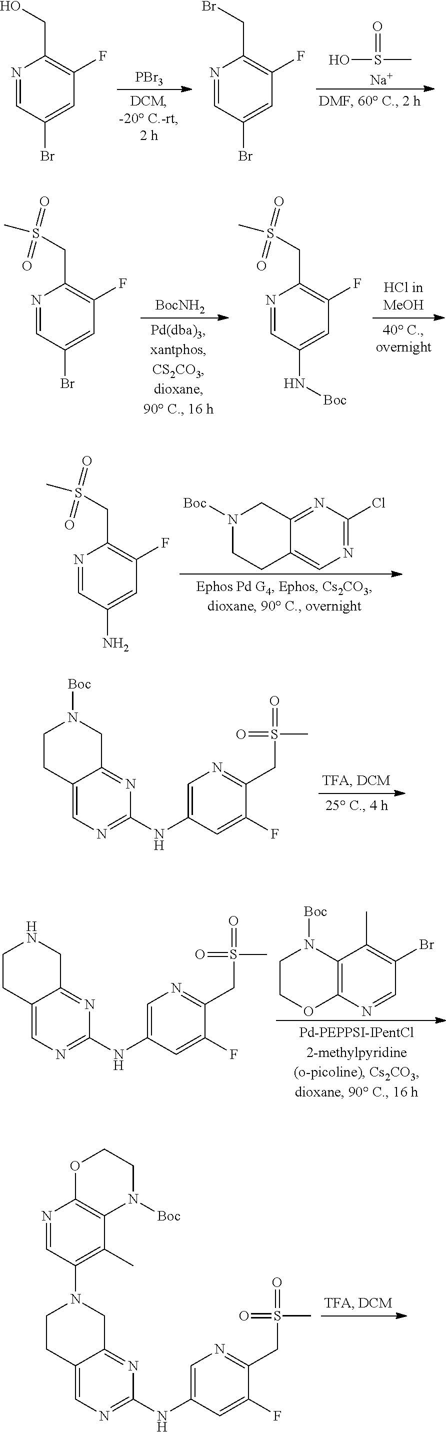

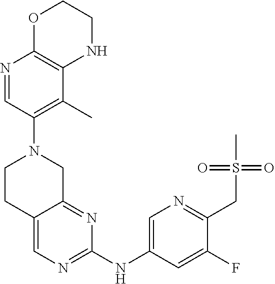 [SMILES: Cc1c(N2CCc3cnc(Nc4cnc(CS(C)(=O)=O)c(F)c4)nc3C2)cnc2c1NCCO2]

5-bromo-2-(bromomethyl)-3-fluoropyridine: To a solution of (5-bromo-3-fluoropyridin-2-yl)methanol (0.99 g; 4.71 mmol) in DCM (30.00 mL) was added PBr₃ (0.61 mL; 6.12 mmol) at −20° C. The resulting mixture was stirred for 2 h at room temperature. The reaction was quenched with sat. NaHCO₃ (aq.) (20 mL) at 0° C. The resulting mixture was extracted with DCM (3×30 mL). The combined organic layers were dried over anhydrous Na₂SO₄. After filtration, the filtrate was concentrated under reduced pressure to afford 5-bromo-2-(bromomethyl)-3-fluoropyridine (670.00 mg; 51.5%)

5-bromo-3-fluoro-2-(methanesulfonylmethyl)pyridine: To a stirred solution of 5-bromo-2-(bromomethyl)-3-fluoropyridine (660.00 mg; 2.39 mmol) in DMF (20.00 mL) was added sodium methanesulfinate (1232.00 mg; 11.95 mmol). The resulting mixture was stirred for 4 h at 60° C. under nitrogen atmosphere. The mixture was allowed to cool down to room temperature and was concentrated under reduced pressure. The residue was purified by silica gel column chromatography and eluted with PE/EtOAc (1:1) to afford 5-bromo-3-fluoro-2-(methanesulfonylmethyl)pyridine (430.00 mg; 64.4%) as a yellow solid.

tert-butyl N-[5-fluoro-6-(methanesulfonylmethyl)pyridin-3-yl]carbamate: To a stirred mixture of 5-bromo-3-fluoro-2-(methanesulfonylmethyl) pyridine (775.00 mg; 2.53 mmol) and tert-butyl carbamate (469.00 mg; 3.80 mmol) in 1,4-dioxane (20.00 mL) were added Pd₂(dba)₃ (245.00 mg; 0.25 mmol), Xantphos (297.00 mg; 0.51 mmol) and Cs₂CO₃ (1738.00 mg; 5.07 mmol) at room temperature. After stirring for 16 h at 90° C. under argon atmosphere, the mixture was concentrated under reduced pressure. The residue was purified by silica gel column chromatography and eluted with DCM/MeOH (19:1) to afford tert-butyl N-[5-fluoro-6-(methanesulfonylmethyl) pyridin-3-yl]carbamate (770.00 mg; 87.5%) as a yellow solid.

5-fluoro-6-(methanesulfonylmethyl) pyridin-3-amine To a stirred solution of tert-butyl N-[5-fluoro-6-(methanesulfonylmethyl) pyridin-3-yl]carbamate: (770.00 mg; 2.22 mmol) was added HCl (g) in MeOH (10.00 mL) in portions at room temperature. The resulting mixture was stirred for overnight at 40° C. under nitrogen atmosphere. The filtrate was concentrated under reduced pressure. The mixture was basified to pH [14] with saturated NaOH (aq.). The resulting mixture was extracted with DCM. The combined organic layers were washed with brine and dried over anhydrous Na₂SO₄. The resulting mixture was concentrated under reduced pressure. The residue was purified by silica gel column chromatography and eluted with DCM/MeOH (6:1) to afford 5-fluoro-6-(methanesulfonylmethyl)pyridin-3-amine (310.00 mg; 67.8%) as a yellow solid.

tert-butyl 2-{[5-fluoro-6-(methanesulfonylmethyl)pyridin-3-yl]amino}-5H,6H,7H,8H-pyrido[3,4-d]pyrimidine-7-carboxylate: To a solution of 5-fluoro-6-(methanesulfonylmethyl)pyridin-3-amine (300.00 mg; 1.46 mmol) and tert-butyl 7-bromo-8-methyl-1H,2H,3H-pyrido[2,3-b][1,4]oxazine-1-carboxylate (513.00 mg; 1.46 mmol) in 1,4-dioxane (20.00 mL) were added Ephos (164.00 mg; 0.29 mmol), Cs₂CO₃ (999.00 mg; 2.91 mmol) and EPhos Pd G₄ (141.00 mg; 0.15 mmol). The resulting mixture was stirred for overnight at 90° C. under nitrogen atmosphere. The resulting mixture was concentrated under reduced pressure. The residue was purified by silica gel column chromatography and eluted with DCM/MeOH (24:1) to afford tert-butyl 2-{[5-fluoro-6-(methanesulfonylmethyl)pyridin-3-yl]amino}-5H,6H,7H,8H-pyrido[3,4-d]pyrimidine-7-carboxylate (610.00 mg; 84.8%) as a yellow solid.

5-fluoro-6-(methanesulfonylmethyl)-N-{5H,6H,7H,8H-pyrido[3,4-d]pyrimidin-2-yl}pyridin-3-amine: To a stirred solution of tert-butyl 2-{[5-fluoro-6-(methanesulfonylmethyl)pyridin-3-yl]amino}-5H,6H,7H,8H-pyrido[3,4-d]pyrimidine-7-carboxylate (400.00 mg; 0.82 mmol) in DCM (10.00 mL) was added TFA (2.00 mL) in portions at room temperature. The resulting mixture was stirred for 4 h at 25° C. under nitrogen atmosphere. The mixture was basified to pH [14] the filter cake was washed with H₂O (2×50 mL) and dried under reduced pressure to afford 5-fluoro-6-(methanesulfonylmethyl)-N-{5H,6H,7H,8H-pyrido[3,4-d]pyrimidin-2-yl}pyridin-3-amine (240.00 mg; 84.7%) as a yellow solid.

tert-butyl 7-(2-{[5-fluoro-6-(methanesulfonylmethyl)pyridin-3-yl]amino}-5H,6H,7H,8H-pyrido[3,4-d]pyrimidin-7-yl)-8-methyl-1H,2H,3H-pyrido[2,3-b][1,4]oxazine-1-carboxylate: To a solution of 5-fluoro-6-(methanesulfonylmethyl)-N-{5H,6H,7H,8H-pyrido[3,4-d]pyrimidin-2-yl}pyridin-3-amine (320.00 mg; 0.93 mmol) and tert-butyl 7-bromo-8-methyl-1H,2H,3H-pyrido[2,3-b][1,4]oxazine-1-carboxylate (328.00 mg; 0.93 mmol) in 1,4-dioxane (20.00 mL) were added Cs₂CO₃ (638.00 mg; 1.86 mmol) and Pd-PEPPSI-IPentCl 2-methylpyridine (o-picoline) (93.00 mg; 0.11 mmol). The resulting mixture was stirred for overnight at 90° C. under nitrogen atmosphere. The resulting mixture was concentrated under reduced pressure. The residue was purified by silica gel column chromatography and eluted with DCM/MeOH (10:1) to afford tert-butyl 7-(2-{[5-fluoro-6-(methanesulfonylmethyl)pyridin-3-yl]amino}-5H,6H,7H,8H-pyrido[3,4-d]pyrimidin-7-yl)-8-methyl-1H,2H,3H-pyrido[2,3-b][1,4]oxazine-1-carboxylate (60.00 mg; 9.9%) as a yellow oil.

5-fluoro-6-(methanesulfonylmethyl)-N-(7-{8-methyl-1H,2H,3H-pyrido[2,3-b][1,4]oxazin-7-yl}-5H,6H,7H,8H-pyrido[3,4-d]pyrimidin-2-yl)pyridin-3-amine: To a stirred solution of tert-butyl 7-(2-{[5-fluoro-6-(methanesulfonylmethyl)pyridin-3-yl]amino}-5H,6H,7H,8H-pyrido[3,4-d]pyrimidin-7-yl)-8-methyl-1H,2H,3H-pyrido[2,3-b][1,4]oxazine-1-carboxylate (55.00 mg; 0.08 mmol) in DCM (5.00 mL) was added TFA (1.00 mL) in portions at room temperature. The mixture was basified to pH [7] with saturated NaHCO₃ (aq.). The resulting mixture was extracted with DCM. The combined organic layers were washed with brine and dried over anhydrous Na₂SO₄. After filtration, the filtrate was concentrated under reduced pressure. The residue was purified by Prep-HPLC with the following conditions (Column: X Bridge Prep OBD C18 Column, 30*150 mm, 5 μm; Mobile Phase A: water (10 mmol/L NH₄HCO₃+0.05% NH₄OH Mobile Phase B: ACN; Flow rate: 60 mL/min.; Gradient: 18% B to 48% B in 9 min., 48% B; Wave Length: 254 nm; RT1 (min.): 7; Number Of Runs: 2) to afford 5-fluoro-6-(methanesulfonylmethyl)-N-(7-{8-methyl-1H,2H,3H-pyrido[2,3-b][1,4]oxazin-7-yl}-5H,6H,7H,8H-pyrido[3,4-d]pyrimidin-2-yl)pyridin-3-amine (20.90 mg; 50.7%) as a yellow solid.

HPLC: 99.5% purity, RT=3.17 min. MS: m/z=486.0 [M+H]⁺. ¹HNMR (300 MHz, DMSO-d6) δ 10.19 (d, J=1.4 Hz, 1H), 8.68 (dd, J=2.2, 1.2 Hz, 1H), 8.43 (s, 1H), 8.35 (dd, J=12.6, 2.1 Hz, 1H), 7.29 (s, 1H), 5.52 (s, 1H), 4.61-4.52 (m, 2H), 4.18 (t, J=4.4 Hz, 2H), 4.01 (s, 2H), 3.30-3.23 (m, 2H), 3.11 (d, J=5.8 Hz, 2H), 3.02 (s, 3H), 2.82 (t, J=5.6 Hz, 2H), 2.03 (s, 3H).

### Example 20: Synthesis of Compound 20 6-(2-methanesulfonylpropan-2-yl)-N-(7-{8-methyl-1H,2H,3H-pyrido[2,3-b][1,4]oxazin-7-yl}-5H,6H,7H,8H-pyrido[3,4-d]pyrimidin-2-yl)pyridin-3-amine

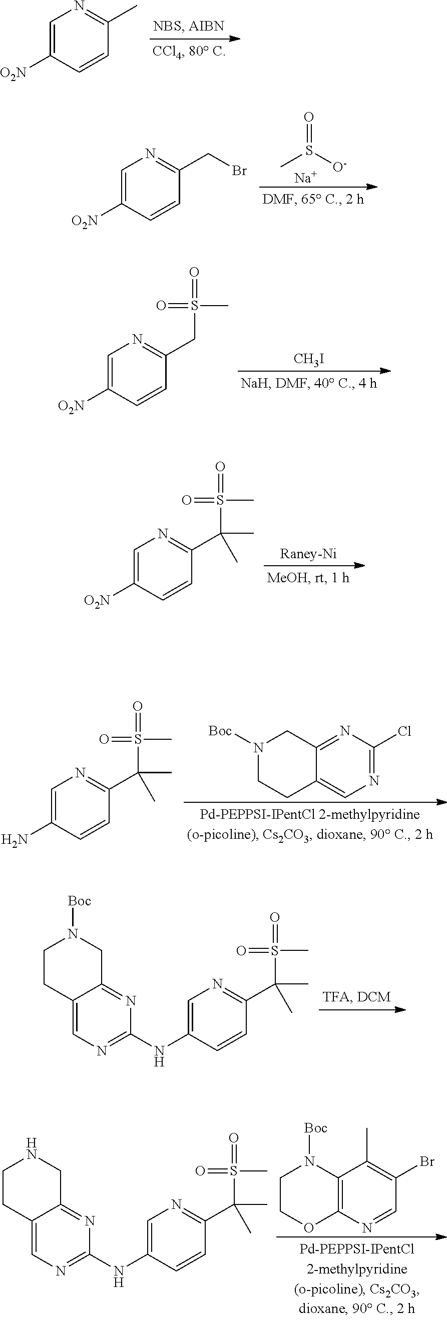

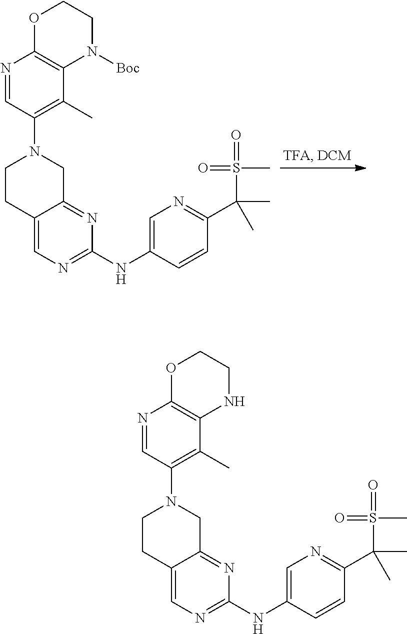 [SMILES: Cc1c(N2CCc3cnc(Nc4ccc(C(C)(C)S(C)(=O)=O)nc4)nc3C2)cnc2c1N(C(=O)OC(C)(C)C)CCO2 ; Cc1c(N2CCc3cnc(Nc4ccc(C(C)(C)S(C)(=O)=O)nc4)nc3C2)cnc2c1NCCO2]

2-(bromomethyl)-5-nitropyridine: To a stirred solution of 2-methyl-5-nitropyridine (10.00 g; 70.95 mmol) in CCl₄ (100.00 mL) was added NBS (38.66 g; 212.85 mmol) and AIBN (4.76 g; 28.38 mmol) in portions at room temperature. After stirring for 16 h at 80° C., the resulting mixture was concentrated under reduced pressure. The residue was purified by silica gel column chromatography and eluted with PE/EA (6:1) to afford 2-(bromomethyl)-5-nitropyridine (6.20 g; 31.65%) as a yellow solid.

2-(methanesulfonylmethyl)-5-nitropyridine: To a stirred solution of 2-(bromomethyl)-5-nitropyridine (5.00 g; 18.11 mmol) in DMF (10.00 mL) was added sodium methanesulfinate (3.73 g; 36.21 mmol). The resulting mixture was stirred for 2 h at 65° C. under nitrogen atmosphere. The resulting mixture was concentrated under reduced pressure. The residue was purified by silica gel column chromatography and eluted with PE/EA (1:1) to afford 2-(methanesulfonylmethyl)-5-nitropyridine (3.25 g; 81.55%) as a yellow solid.

2-(2-methanesulfonylpropan-2-yl)-5-nitropyridine: To a solution of 2-(methanesulfonylmethyl)-5-nitropyridine (1.25 g; 5.68 mmol) in DMF (12.00 mL) was added NaH (0.27 g; 6.81 mmol) and CH₃I (2.12 g; 14.20 mmol) at ° C. After stirring for 4 h at 40° C., the resulting mixture was concentrated under reduced pressure. The residue was purified by silica gel column chromatography and eluted with PE/EtOAc (1:1) to afford 2-(2-methanesulfonylpropan-2-yl)-5-nitropyridine (270.00 mg; 19.44%) as a yellow solid.

6-(2-methanesulfonylpropan-2-yl)pyridin-3-amine: To a solution of 2-(2-methanesulfonylpropan-2-yl)-5-nitropyridine (270.00 mg; 1.10 mmol) in MeOH (15.00 mL) was added Raney-Ni (114.49 mg; 1.27 mmol) at room temperature. After stirring for 1 h at room temperature under hydrogen atmosphere, the resulting mixture was filtered. The filter cake was washed with MeOH and then filtrate was concentrated under reduced pressure to afford 6-(2-methanesulfonylpropan-2-yl)pyridin-3-amine (217.00 mg; 88.26%) as brown oil.

tert-butyl 2-{[6-(2-methanesulfonylpropan-2-yl)pyridin-3-yl]amino}-5H,6H,7H,8H-pyrido[3,4-d]pyrimidine-7-carboxylate: To a stirred solution of 6-(2-methanesulfonylpropan-2-yl)pyridin-3-amine (210.00 mg; 0.94 mmol) and tert-butyl 2-chloro-5H,6H,7H,8H-pyrido[3,4-d]pyrimidine-7-carboxylate (257.25 mg; 0.94 mmol) in 1,4-dioxane (10.00 mL) was added Pd-PEPPSI-IPentCl 2-methylpyridine (o-picoline) (83.39 mg; 0.09 mmol) and Cs₂CO₃ (646.81 mg; 1.89 mmol). The resulting mixture was stirred for 3 h at 100° C. under nitrogen atmosphere. The resulting mixture was concentrated under reduced pressure. The residue was purified by silica gel column chromatography and eluted with PE/EtOAc (1:1) to afford tert-butyl 2-{[6-(2-methanesulfonylpropan-2-yl)pyridin-3-yl]amino}-5H,6H,7H,8H-pyrido[3,4-d]pyrimidine-7-carboxylate (120.00 mg; 27.11%) as a yellow oil.

6-(2-methanesulfonylpropan-2-yl)-N-{5H,6H,7H,8H-pyrido[3,4-d]pyrimidin-2-yl}pyridin-3-amine: To a stirred solution of tert-butyl 2-{[6-(2-methanesulfonylpropan-2-yl)pyridin-3-yl]amino}-5H,6H,7H,8H-pyrido[3,4-d]pyrimidine-7-carboxylate (120.00 mg; 0.26 mmol) in DCM (10.00 mL) was added TFA (2.00 mL) in portions at room temperature. After stirring for 1 h at room temperature, the resulting mixture was concentrated under reduced pressure to afford 6-(2-methanesulfonylpropan-2-yl)-N-{5H,6H,7H,8H-pyrido[3,4-d]pyrimidin-2-yl}pyridin-3-amine (80.00 mg; 86.46%) as a yellow oil.

tert-butyl 7-(2-{[6-(2-methanesulfonylpropan-2-yl)pyridin-3-yl]amino}-5H,6H,7H,8H-pyrido[3,4-d]pyrimidin-7-yl)-8-methyl-1H,2H,3H-pyrido[2,3-b][1,4]oxazine-1-carboxylate: To a stirred solution of 6-(2-methanesulfonylpropan-2-yl)-N-{5H,6H,7H,8H-pyrido[3,4-d]pyrimidin-2-yl}pyridin-3-amine (120.00 mg; 0.33 mmol) and tert-butyl 7-bromo-8-methyl-1H,2H,3H-pyrido[2,3-b][1,4]oxazine-1-carboxylate (114.78 mg; 0.33 mmol) in 1,4-dioxane (10.00 mL) was added Pd-PEPPSI-IPentCl 2-methylpyridine (o-picoline) (29.32 mg; 0.03 mmol) and Cs₂CO₃ (227.42 mg; 0.66 mmol). The resulting mixture was stirred for 3 h at 100° C. under nitrogen atmosphere. The resulting mixture was concentrated under reduced pressure. The residue was purified by silica gel column chromatography and eluted with CH₂Cl₂/MeOH (10:1) to afford tert-butyl 7-(2-{[6-(2-methanesulfonylpropan-2-yl)pyridin-3-yl]amino}-5H,6H,7H,8H-pyrido[3,4-d]pyrimidin-7-yl)-8-methyl-1H,2H,3H-pyrido[2,3-b][1,4]oxazine-1-carboxylate (47.00 mg; 21.78%) as a yellow oil.

6-(2-methanesulfonylpropan-2-yl)-N-(7-{8-methyl-1H,2H,3H-pyrido[2,3-b][1,4]oxazin-7-yl}-5H,6H,7H,8H-pyrido[3,4-d]pyrimidin-2-yl)pyridin-3-amine: To a stirred solution of tert-butyl 7-(2-{[6-(2-methanesulfonylpropan-2-yl)pyridin-3-yl]amino}-5H,6H,7H,8H-pyrido[3,4-d]pyrimidin-7-yl)-8-methyl-1H,2H,3H-pyrido[2,3-b][1,4]oxazine-1-carboxylate (47.00 mg; 0.07 mmol) in DCM (5.00 mL) was added TFA (1.00 mL) in portions at room temperature. After stirring for 1 h at room temperature, the resulting mixture was concentrated under reduced pressure. The crude product was purified by Prep-HPLC with the following conditions: Column: X Bridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 20% B to 50% B in 9 min., 50% B; Wave Length: 254 nm; RT1 (min.): 7; Number Of Runs: 2, to afford 6-(2-methanesulfonylpropan-2-yl)-N-(7-{8-methyl-1H,2H,3H-pyrido[2,3-b][1,4]oxazin-7-yl}-5H,6H,7H,8H-pyrido[3,4-d]pyrimidin-2-yl)pyridin-3-amine (7.10 mg; 19.51%) as a yellow solid.

HPLC: 98.3% purity, RT=3.19 min. MS: m/z=496.2 [M+H]⁺. ¹H NMR (300 MHz, DMSO-d6): 9.89 (s, 1H), 8.95 (d, J=2.7 Hz, 1H), 8.40 (s, 1H), 8.25 (dd, J=8.8, 2.7 Hz, 1H), 7.58 (d, J=8.8 Hz, 1H), 7.29 (s, 1H), 5.54 (s, 1H), 4.20 (d, J=4.8 Hz, 2H), 4.01 (s, 2H), 3.43 (s, 2H), 3.13 (s, 2H), 2.80 (d, J=14.5 Hz, 5H), 2.05 (s, 3H), 1.75 (s, 6H).

### Example 21: Synthesis of Compound 21 6-(methanesulfonylmethyl)-5-methyl-N-(7-{8-methyl-1H,2H,3H-pyrido[2,3-b][1,4]oxazin-7-yl}-5H,6H,7H,8H-pyrido[3,4-d]pyrimidin-2-yl)pyridin-3-amine

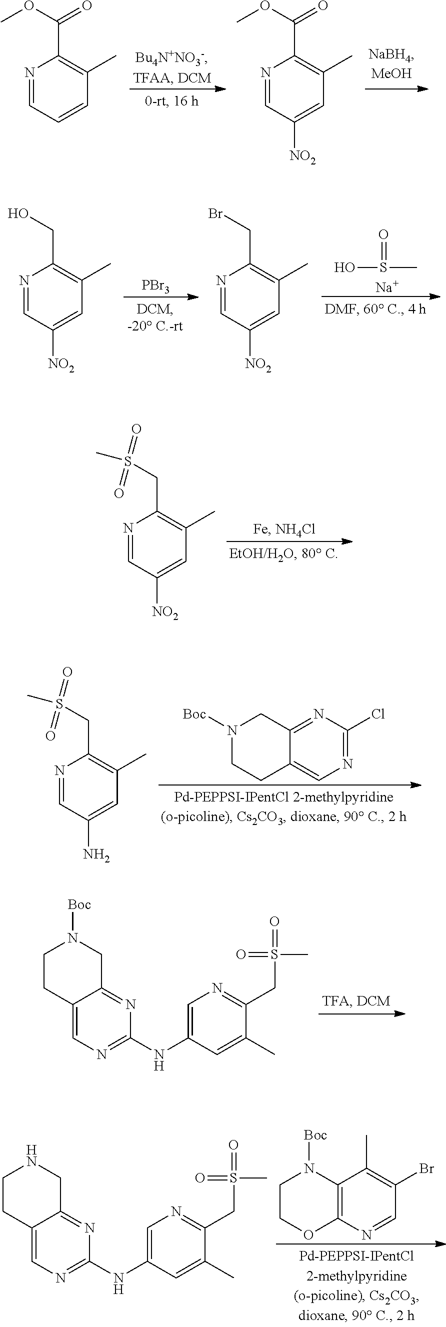

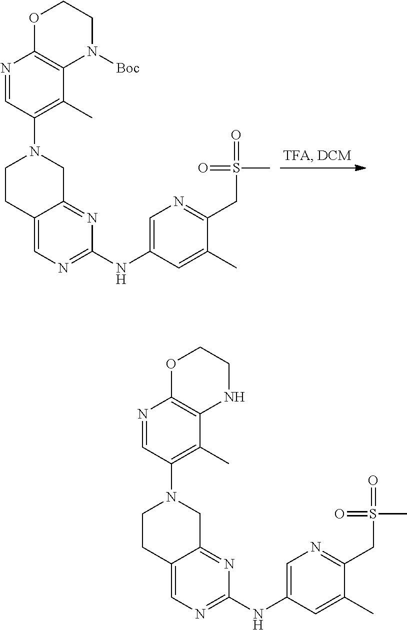 [SMILES: Cc1cc(Nc2ncc3c(n2)CN(c2cnc4c(c2C)N(C(=O)OC(C)(C)C)CCO4)CC3)cnc1CS(C)(=O)=O ; Cc1cc(Nc2ncc3c(n2)CN(c2cnc4c(c2C)NCCO4)CC3)cnc1CS(C)(=O)=O]

methyl 3-methyl-5-nitropyridine-2-carboxylate: To a stirred solution of methyl 3-methylpyridine-2-carboxylate (5.00 g; 31.42 mmol) in DCM (10.00 mL) was added TFAA (13.20 g; 59.71 mmol) and nitro-3-oxidanide; tetrabutylazanium (15.10 g; 47.11 mmol) at 0° C. under nitrogen atmosphere. Then the resulting mixture was stirred for overnight at room temperature. The resulting mixture was concentrated under vacuum. The residue was purified by silica gel column chromatography and eluted with PE/EtOAc (1:7) to afford methyl 3-methyl-5-nitropyridine-2-carboxylate (3.80 g; 55.8%) as a yellow solid.

(3-methyl-5-nitropyridin-2-yl)methanol: To a stirred solution of methyl 3-methyl-5-nitropyridine-2-carboxylate (3.40 g; 15.70 mmol) in DCM (60.00 mL) was added diisobutylaluminum hydride (1.0M in dichloromethane; 32.00 mL; 32.00 mmol) at 0° C. under nitrogen atmosphere. The resulting mixture was stirred for 3 h at room temperature. The reaction was quenched by the addition of H₂O (3 mL) at 0° C. The resulting mixture was filtered, and the filter cake was washed with EA (3×20 mL). The filtrate was concentrated under reduced pressure. The residue was purified by silica gel column chromatography and eluted with PE/EA (2:1) to afford (3-methyl-5-nitropyridin-2-yl)methanol (350.00 mg; 11.7%) as a yellow solid.

2-(bromomethyl)-3-methyl-5-nitropyridine: To a solution of (3-methyl-5-nitropyridin-2-yl)methanol (320.00 mg; 1.67 mmol) in DCM (3.00 mL) was added PBr₃ (0.20 mL; 2.00 mmol) at −20° C. The resulting mixture was stirred for 2 h at room temperature. The reaction was quenched with sat. NaHCO₃ (aq.) (20 mL) at 0° C. The resulting mixture was extracted with DCM (3×30 mL). The combined organic layers were dried over anhydrous Na₂SO₄. After filtration, the filtrate was concentrated under reduced pressure to afford 2-(bromomethyl)-3-methyl-5-nitropyridine (440.00 mg; 1.61 mmol; 96.5%) as a red solid.

2-(methanesulfonylmethyl)-3-methyl-5-nitropyridine: To a stirred solution of 2-(bromomethyl)-3-methyl-5-nitropyridine (430.00 mg; 1.57 mmol) in DMF (10.00 mL) was added sodium methanesulfinate (325.00 mg; 3.15 mmol). The resulting mixture was stirred for 4 h at 60° C. under nitrogen atmosphere. The mixture was allowed to cool down to room temperature and was concentrated under reduced pressure. The residue was purified by silica gel column chromatography and eluted with PE/EtOAc (1:1) to afford 2-(methanesulfonylmethyl)-3-methyl-5-nitropyridine (210.00 mg; 52.1%) as a yellow oil.

6-(methanesulfonylmethyl)-5-methylpyridin-3-amine: To a stirred solution of 2-(methanesulfonylmethyl)-3-methyl-5-nitropyridine (300.00 mg; 1.17 mmol) and NH₄Cl (264.00 mg; 4.69 mmol) in water (10.00 mL) and EtOH (15.00 mL) were added Fe (345.00 mg; 5.85 mmol) in portions at room temperature. The resulting mixture was stirred for 2 h at 80° C. The resulting mixture was filtered, and the filter cake was washed with EtOAc (100 mL). The filtrate was extracted with EtOAc (100 mL*3). The combined organic layers were washed with brine (100 mL) and dried over anhydrous Na₂SO₄. After filtration, the filtrate was concentrated under reduced pressure to afford 6-(methanesulfonylmethyl)-5-methylpyridin-3-amine (240.00 mg; 96.0%) as a black oil.

tert-butyl 2-{[6-(methanesulfonylmethyl)-5-methylpyridin-3-yl]amino}-5H,6H,7H,8H-pyrido[3,4-d]pyrimidine-7-carboxylate: To a solution of tert-butyl 2-chloro-5H,6H,7H,8H-pyrido[3,4-d]pyrimidine-7-carboxylate (278.00 mg; 1.03 mmol) and 6-(methanesulfonylmethyl)-5-methylpyridin-3-amine (220.00 mg; 1.03 mmol) in 1,4-dioxane (20.00 mL) were added Cs₂CO₃ (707.00 mg; 2.06 mmol), and Pd-PEPPSI-IPentCl 2-methylpyridine (o-picoline) (92.00 mg; 0.10 mmol). The resulting mixture was stirred for overnight at 90° C. under nitrogen atmosphere. The resulting mixture was concentrated under reduced pressure. The residue was purified by silica gel column chromatography and eluted with PE/EA (1:9) to afford tert-butyl 2-{[6-(methanesulfonylmethyl)-5-methylpyridin-3-yl]amino}-5H,6H,7H,8H-pyrido [3,4-d]pyrimidine-7-carboxylate (250.00 mg; 50.7%) as a yellow oil.

6-(methanesulfonylmethyl)-5-methyl-N-{5H,6H,7H,8H-pyrido[3,4-d]pyrimidin-2-yl}pyridin-3-amine: To a stirred solution of tert-butyl 2-{[6-(methanesulfonylmethyl)-5-methylpyridin-3-yl]amino}-5H,6H,7H,8H-pyrido[3,4-d]pyrimidine-7-carboxylate (240.00 mg; 0.50 mmol) in DCM (10.00 mL) was added TFA (2.00 mL) in portions at room temperature. The mixture was basified to pH [7] with saturated NaHCO₃ (aq.). The residue was purified by silica gel column chromatography and eluted with DCM/MeOH (4:1) to afford 6-(methanesulfonylmethyl)-5-methyl-N-{5H,6H,7H,8H-pyrido[3,4-d]pyrimidin-2-yl}pyridin-3-amine (170.00 mg; 95.1%) as a yellow oil.

tert-butyl 7-(2-{[6-(methanesulfonylmethyl)-5-methylpyridin-3-yl]amino}-5H,6H,7H,8H-pyrido[3,4-d]pyrimidin-7-yl)-8-methyl-1H,2H,3H-pyrido[2,3-b][1,4]oxazine-1-carboxylate: To a solution of 6-(methanesulfonylmethyl)-5-methyl-N-{5H,6H,7H,8H-pyrido[3,4-d]pyrimidin-2-yl}pyridin-3-amine (160.00 mg; 0.45 mmol) and tert-butyl 7-bromo-8-methyl-1H,2H,3H-pyrido[2,3-b][1,4]oxazine-1-carboxylate (159.00 mg; 0.45 mmol) in 1,4-dioxane (10.00 mL) were added Cs₂CO₃ (309.00 mg; 0.90 mmol) and Pd-PEPPSI-IPentCl 2-methylpyridine (o-picoline) (41.00 mg; 0.05 mmol). The resulting mixture was stirred for overnight at 90° C. under nitrogen atmosphere. The resulting mixture was concentrated under reduced pressure. The residue was purified by silica gel column chromatography and eluted with DCM/MeOH (10:1) to afford tert-butyl 7-(2-{[6-(methanesulfonylmethyl)-5-methylpyridin-3-yl]amino}-5H,6H,7H,8H-pyrido[3,4-d]pyrimidin-7-yl)-8-methyl-1H,2H,3H-pyrido[2,3-b][1,4]oxazine-1-carboxylate (120.00 mg; 36.4%) as a yellow oil.

6-(methanesulfonylmethyl)-5-methyl-N-(7-{8-methyl-1H,2H,3H-pyrido[2,3-b][1,4]oxazin-7-yl}-5H,6H,7H,8H-pyrido[3,4-d]pyrimidin-2-yl)pyridin-3-amine: To a stirred solution of tert-butyl 7-(2-{[6-(methanesulfonylmethyl)-5-methylpyridin-3-yl]amino}-5H,6H,7H,8H-pyrido[3,4-d]pyrimidin-7-yl)-8-methyl-1H,2H,3H-pyrido[2,3-b][1,4]oxazine-1-carboxylate (110.00 mg; 0.15 mmol) in DCM (10.00 mL) was added TFA (2.00 mL) in portions at room temperature. The resulting mixture was concentrated under reduced pressure. The crude product was purified by Prep-HPLC with the following conditions (Column: XBridge Prep OBD C18 Column, 30*150 mm, 5 μm; Mobile Phase A: water (10 mmol/L NH₄HCO₃+0.1% NH₄OH), Mobile Phase B: ACN; Flow rate: 60 mL/min.; Gradient: 18% B to 48% B in 9 min., 48% B; Wave Length: 254 nm; RTT (min.): 7) to afford 6-(methanesulfonylmethyl)-5-methyl-N-(7-{8-methyl-1H,2H,3H-pyrido[2,3-b][1,4]oxazin-7-yl}-5H,6H,7H,8H-pyrido[3,4-d]pyrimidin-2-yl)pyridin-3-amine (20.10 mg; 27.7%) as a yellow solid.

HPLC: 99.8% purity, RT=2.67 min. MS: m/z=482.0[M+H]⁺. ¹H NMR (300 MHz, DMSO-d6) δ 9.80 (s, 1H), 8.79 (d, J=2.5 Hz, 1H), 8.40 (s, 1H), 8.07 (d, J=2.5 Hz, 1H), 7.30 (s, 1H), 5.53 (t, J=2.5 Hz, 1H), 4.55 (s, 2H), 4.20 (t, J=4.2 Hz, 2H), 4.00 (s, 2H), 3.33 (s, 2H), 3.13 (t, J=5.6 Hz, 2H), 2.98 (s, 3H), 2.82 (t, J=5.6 Hz, 2H), 2.37 (s, 3H), 2.05 (s, 3H).

### Example 22: Synthesis of Compound 22 6-methyl-2-[(7-{8-methyl-1H,2H,3H-pyrido[2,3-b][1,4]oxazin-7-yl}-5H,6H,7H,8H-pyrido[3,4-d]pyrimidin-2-yl)amino]-4H,5H,6H,7H,8H-pyrazolo[1,5-d][1,4]diazepin-7-on

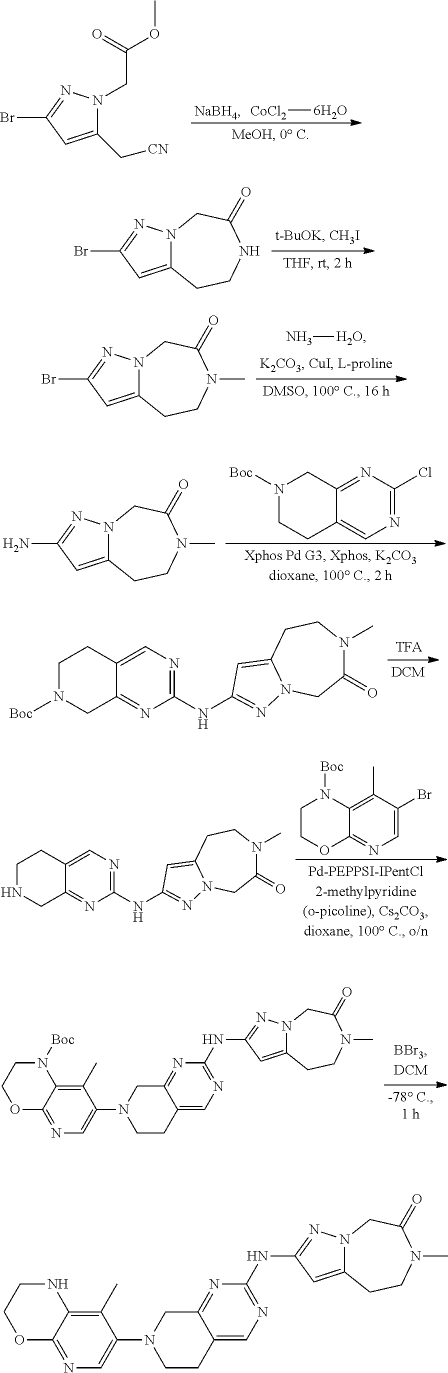

2-bromo-4H,5H,6H,7H,8H-pyrazolo[1,5-d][1,4]diazepin-7-one: To a stirred solution of methyl 2-[3-bromo-5-(cyanomethyl)-1H-pyrazol-1-yl]acetate (1.50 g; 5.06 mmol) and Cobalt chloride hexahydrate (2.46 g; 10.11 mmol) in MeOH (50.00 mL) was added NaBH4 (1.06 g; 25.28 mmol) in portions at 0° C. under N2 atmosphere. The resulting mixture was stirred for 4 h at 25° C. under N2 atmosphere. The reaction was quenched with water (50 mL) and the resulting mixture was concentrated under reduced pressure. The mixture was extracted with EtOAc (3×50 mL), the combined organic layers was dried over anhydrous Na₂SO₄ and concentrated under reduced pressure to afford 2-bromo-4H,5H,6H,7H,8H-pyrazolo[1,5-d][1,4]diazepin-7-one (1.10 g; 64.27%) as a white solid.

2-bromo-6-methyl-4H,5H,6H,7H,8H-pyrazolo[1,5-d][1,4]diazepin-7-one: To a stirred solution of 2-bromo-4H,5H,6H,7H,8H-pyrazolo[1,5-d][1,4]diazepin-7-one (1.10 g; 3.07 mmol) and MeI (0.40 mL; 6.15 mmol) in THF (15.00 mL) was added potassium tert-butoxide solution 1.0 M in THF (5.99 mL; 6.15 mmol) at room temperature under N2 atmosphere. The resulting mixture was stirred for 3 h at 25° C. under N2 atmosphere. The resulting mixture was diluted with water (50 mL). The solution was extracted with EtOAc (3×30 mL). The combined organic layers were washed with brine dried over anhydrous Na₂SO₄ and concentrated under reduced pressure. The residue was purified by C18 flash eluted with 67% ACN in water to afford 2-bromo-6-methyl-4H,5H,6H,7H,8H-pyrazolo[1,5-d][1,4]diazepin-7-one (700.00 mg; 91.38%) as a white solid.

2-amino-6-methyl-4H,5H,6H,7H,8H-pyrazolo[1,5-d][1,4]diazepin-7-one: To a stirred solution of 2-bromo-6-methyl-4H,5H,6H,7H,8H-pyrazolo[1,5-d][1,4]diazepin-7-one (240.00 mg; 0.96 mmol) and potassium carboperoxoate (280.11 mg; 1.93 mmol) in DMSO (3.00 mL) and NH₄OH (0.6 mL) were added CuI (38.60 mg; 0.19 mmol) and L-Proline (46.67 mg; 0.39 mmol) in portions at room temperature under N₂ atmosphere. The resulting mixture was stirred for overnight at 110° C. under N₂ atmosphere using a sealed tube. The residue was purified by C18 flash column and eluted with 30% ACN in water to afford crude product. The crude product was repurified by silica gel column and eluted with 5% MeOH in DCM to afford 2-amino-6-methyl-4H,5H,6H,7H,8H-pyrazolo[1,5-d][1,4]diazepin-7-one (50.00 mg; 27.67%) as a white solid.

tert-butyl 2-({6-methyl-7-oxo-4H,5H,6H,7H,8H-pyrazolo[1,5-d][1,4]diazepin-2-yl}amino)-5H,6H,7H,8H-pyrido[3,4-d]pyrimidine-7-carboxylate: To a stirred solution of 2-amino-6-methyl-4H,5H,6H,7H,8H-pyrazolo[1,5-d][1,4]diazepin-7-one (500.00 mg; 2.66 mmol) and tert-butyl 2-chloro-5H,6H,7H,8H-pyrido[3,4-d]pyrimidine-7-carboxylate (756.00 mg; 2.66 mmol) in N,N-dimethylformamide (5.00 mL) were added XPhos Pd G₃ (237.00 mg; 0.27 mmol) and X-PHOS (267.00 mg; 0.53 mmol) and potassium carbonate (1.16 g; 7.99 mmol) in portions at room temperature under N₂ atmosphere. The resulting mixture was stirred for 1 h at 100° C. under N₂ atmosphere. The resulting mixture was diluted with water (15 mL) extracted with EtOAc (3×20 mL). The combined organic layers were washed with brine and dried over anhydrous Na₂SO₄. The resulting mixture was concentrated under reduced pressure. The residue was purified by silica gel column chromatography and eluted with (DCM:MeOH=10:1) to afford tert-butyl 2-({6-methyl-7-oxo-4H,5H,6H,7H,8H-pyrazolo[1,5-d][1,4]diazepin-2-yl}amino)-5H,6H,7H,8H-pyrido[3,4-d]pyrimidine-7-carboxylate (600.00 mg; 49.6%).

6-methyl-2-({5H,6H,7H,8H-pyrido[3,4-d]pyrimidin-2-yl}amino)-4H,5H,6H,7H,8H-pyrazolo[1,5-d][1,4]diazepin-7-one: To a stirred solution of tert-butyl 2-({6-methyl-7-oxo-4H,5H,6H,7H,8H-pyrazolo[1,5-d][1,4]diazepin-2-yl}amino)-5H,6H,7H,8H-pyrido[3,4-d]pyrimidine-7-carboxylate (580.00 mg; 1.28 mmol) in dichloromethane (6.00 mL) was added TFA (2.00 mL) dropwise at 0° C. under N₂ atmosphere. The resulting mixture was stirred for 30 min. at room temperature under N₂ atmosphere. The resulting mixture was concentrated under reduced pressure to afford 6-methyl-2-({5H,6H,7H,8H-pyrido[3,4-d]pyrimidin-2-yl}a.o)-4H,5H,6H,7H,8H-pyrazolo[1,5-d][1,4]diazepin-7-one (400.00 mg; 97.1%) as a yellow oil.

tert-butyl 8-methyl-7-[2-({6-methyl-7-oxo-4H,5H,6H,7H,8H-pyrazolo[1,5-d][1,4]diazepin-2-yl}amino)-5H,6H,7H,8H-pyrido[3,4-d]pyrimidin-7-yl]-1H,2H,3H-pyrido[2,3-b][1,4]oxazine-1-carboxylate: To a stirred solution of 6-methyl-2-({5H,6H,7H,8H-pyrido[3,4-d]pyrimidin-2-yl}amino)-4H,5H,6H,7H,8H-pyrazolo[1,5-d][1,4]diazepin-7-one (150.00 mg; 0.45 mmol) in 1,4-dioxane (3.00 mL) were added tert-butyl 7-bromo-8-methyl-1H,2H,3H-pyrido[2,3-b][1,4]oxazine-1-carboxylate (311.87 mg; 0.91 mmol), Pd-PEPPSI-IPentCl 2-methylpyridine (o-picoline) (40.22 mg; 0.05 mmol) and Cs₂CO₃ (467.89 mg; 1.36 mmol) in portions at room temperature under N₂ atmosphere. The resulting mixture was stirred for overnight at 100° C. under N₂ atmosphere. The residue was purified by silica gel column and eluted with 8% MeOH in DCM to afford tert-butyl 8-methyl-7-[2-({6-methyl-7-oxo-4H,5H,6H,7H,8H-pyrazolo[1,5-d][1,4]diazepin-2-yl}amino)-5H,6H,7H,8H-pyrido[3,4-d]pyrimidin-7-yl]-1H,2H,3H-pyrido[2,3-b][1,4]oxazine-1-carboxylate (90.00 mg; 30.7%) as a yellow oil.

6-methyl-2-[(7-{8-methyl-1H,2H,3H-pyrido[2,3-b][1,4]oxazin-7-yl}-5H,6H,7H,8H-pyrido[3,4-d]pyrimidin-2-yl) amino]-4H,5H,6H,7H,8H-pyrazolo[1,5-d][1,4]diazepin-7-one: To a stirred solution of tert-butyl 8-methyl-7-[2-({6-methyl-7-oxo-4H,5H,6H,7H,8H-pyrazolo[1,5-d][1,4]diazepin-2-yl}amino)-5H,6H,7H,8H-pyrido[3,4-d]pyrimidin-7-yl]-1H,2H,3H-pyrido[2,3-b][1,4]oxazine-1-carboxylate (70.00 mg; 0.11 mmol) in DCM (3.00 mL) was added BBr₃ in DCM (0.22 mL; 0.22 mmol) in portions at −78° C. under N₂ atmosphere. The resulting mixture was stirred for 0.5 h at −78° C. under N₂ atmosphere. The resulting mixture was quenched with NaHCO₃, then diluted with water (15 mL), extracted with DCM (3×10 mL). The combined organic layers were washed with brine (15 mL) and dried over anhydrous Na₂SO₄. The resulting mixture was concentrated under reduced pressure. The residue was purified by Prep-HPLC (Column: XBridge Prep OBD C18 Column, 30*150 mm, 5 μm; Mobile Phase A: water (10 mmol/L NH₄HCO₃+0.1% NH₄OH), Mobile Phase B: ACN; Flow rate: 60 mL/min.; Gradient: 25% B to 55% B in 8 min., 55% B; Wave Length: 254 nm; RTT (min.): 7; Number Of Runs:1) to afford 6-methyl-2-[(7-{8-methyl-1H,2H,3H-pyrido[2,3-b][1,4]oxazin-7-yl}-5H,6H,7H,8H-pyrido[3,4-d]pyrimidin-2-yl)amino] 4H,5H,6H,7H,8H-pyrazolo[1,5-d][1,4]diazepin-7-one (15.50 mg; 29.9%) as a yellow solid.

HPLC: 99.9% purity, RT=2.54 min. MS: m/z=462.3[M+H]⁺. ¹H NMR (400 MHz, DMSO-d6) δ 9.50 (s, 1H), 8.26 (s, 1H), 7.26 (s, 1H), 6.53 (s, 1H), 5.52 (d, J=3.1 Hz, 1H), 4.90 (s, 2H), 4.19 (t, J=4.4 Hz, 2H), 3.91 (s, 2H), 3.86-3.78 (m, 2H), 3.10 (t, J=5.5 Hz, 2H), 3.04 (t, J=5.7 Hz, 4H), 2.94 (s, 3H), 2.76 (s, 2H), 2.03 (s, 3H).

### Example 23: Synthesis of Compound 23 2-methyl-N4-(7-{8-methyl-1H,2H,3H-pyrido[2,3-b][1,4]oxazin-7-yl}-5H,6H,7H,8H-pyrido[3,4-d]pyrimidin-2-yl)-N1-[2-(morpholin-4-yl)ethyl]benzene-1,4-diamine

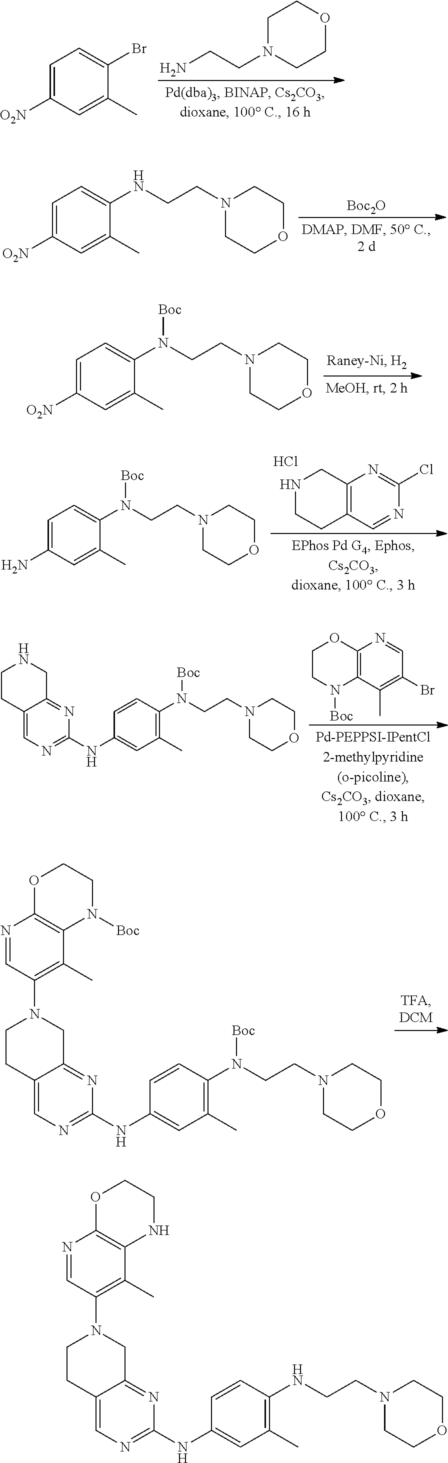

2-methyl-N-[2-(morpholin-4-yl)ethyl]-4-nitroaniline: To a stirred mixture of 2-(morpholin-4-yl)ethan-1-amine (1.86 g; 13.61 mmol) and 1-bromo-2-methyl-4-nitrobenzene (2.00 g; 9.07 mmol) in 1,4-dioxane (30.00 mL) and were added Pd₂(dba)₃ (0.87 g; 0.91 mmol), BINAP (1.19 g; 1.81 mmol) and Cs₂CO₃ (6.22 g; 18.15 mmol) in portions at 25° C. The resulting mixture was stirred for 3 h at 100° C. under argon atmosphere. The resulting mixture was concentrated under reduced pressure. The residue was purified by silica gel column chromatography and eluted with PE/EA (3:1) to afford 2-methyl-N-[2-(morpholin-4-yl)ethyl]-4-nitroaniline (1.90 g; 75.46%) as a yellow solid.

tert-butyl N-(2-methyl-4-nitrophenyl)-N-[2-(morpholin-4-yl)ethyl]carbamate: To a stirred solution of 2-methyl-N-[2-(morpholin-4-yl)ethyl]-4-nitroaniline (1.80 g; 6.49 mmol) and Di-tert-butyl dicarbonate (4.47 g; 19.46 mmol) in DMF (25.00 mL) was added N,N-dimethylpyridin-4-amine (0.17 g; 1.30 mmol) in portions at room temperature under argon atmosphere. The resulting mixture was stirred for 2 days at 50° C. under argon atmosphere. The resulting mixture was concentrated under reduced pressure. The residue was purified by silica gel column and eluted with 15% EtOAc in PE to afford tert-butyl N-(2-methyl-4-nitrophenyl)-N-[2-(morpholin-4-yl)ethyl]carbamate (400.00 mg; 15.73%) as a yellow solid.

tert-butyl N-(4-amino-2-methylphenyl)-N-[2-(morpholin-4-yl)ethyl]carbamate: To a solution of tert-butyl N-(2-methyl-4-nitrophenyl)-N-[2-(morpholin-4-yl)ethyl]carbamate (320.00 mg; 0.82 mmol) in MeOH (10.00 mL) was added Raney-Ni (209.76 mg; 0.24 mmol) at room temperature. The mixture was stirred at room temperature for 2 h under H₂ atmosphere. The resulting mixture was filtered and the filter cake was washed with MeOH (50 mL), the filtrate was concentrated under reduced pressure to afford tert-butyl N-(4-amino-2-methylphenyl)-N-[2-(morpholin-4-yl)ethyl]carbamate (340.00 mg; 94.76%) as a brown oil.

tert-butyl N-[2-methyl-4-({5H,6H,7H,8H-pyrido[3,4-d]pyrimidin-2-yl}amino)phenyl]-N-[2-(morpholin-4-yl)ethyl]carbamate: To a solution of tert-butyl N-(4-amino-2-methylphenyl)-N-[2-(morpholin-4-yl)ethyl]carbamate (150.00 mg; 0.34 mmol) and 2-chloro-5H,6H,7H,8H-pyrido[3,4-d]pyrimidine hydrochloride (112.04 mg; 0.51 mmol) in 1,4-dioxane (10.00 mL) were added Cs₂CO₃ (351.01 mg; 1.02 mmol), Ephos (38.41 mg; 0.07 mmol) and EPhos Pd G₄ (32.99 mg; 0.03 mmol). The resulting mixture was stirred for 3 h at 100° C. under nitrogen atmosphere. The mixture was concentrated under reduced pressure. The residue was purified by silica gel column chromatography and eluted with CH₂Cl₂/MeOH (12:1) to afford tert-butyl N-[2-methyl-4-({5H,6H,7H,8H-pyrido[3,4-d]pyrimidin-2-yl}amino)phenyl]-N-[2-(morpholin-4-yl)ethyl]carbamate (140.00 mg; 75.33%) as a yellow oil.

tert-butyl 7-{2-[(4-{[(tert-butoxy)carbonyl][2-(morpholin-4-yl)ethyl]amino}-3-methylphenyl)amino]-5H,6H,7H,8H-pyrido[3,4-d]pyrimidin-7-yl}-8-methyl-1H,2H,3H-pyrido[2,3-b][1,4]oxazine-1-carboxylate: To a solution of tert-butyl N-[2-methyl-4-({5H,6H,7H,8H-pyrido[3,4-d]pyrimidin-2-yl}amino)phenyl]-N-[2-(morpholin-4-yl)ethyl]carbamate (130.00 mg; 0.24 mmol) and tert-butyl 7-bromo-8-methyl-1H,2H,3H-pyrido[2,3-b][1,4]oxazine-1-carboxylate (119.17 mg; 0.36 mmol) in 1,4-dioxane (10.00 mL) were added Cs₂CO₃ (245.51 mg; 0.72 mmol) and Pd-PEPPSI-IPentCl 2-methylpyridine (o-picoline) (21.10 mg; 0.02 mmol). The resulting mixture was stirred for 3 h at 100° C. under nitrogen atmosphere. The mixture was concentrated under reduced pressure. The residue was purified by silica gel column chromatography and eluted with DCM/MeOH (12:1) to afford tert-butyl 7-{2-[(4-{[(tert-butoxy)carbonyl][2-(morpholin-4-yl)ethyl]amino}-3-methylphenyl)amino]-5H,6H,7H,8H-pyrido[3,4-d]pyrimidin-7-yl}-8-methyl-1H,2H,3H-pyrido[2,3-b][1,4]oxazine-1-carboxylate (120.00 mg; 54.32%) as a yellow oil.

2-methyl-N4-(7-{8-methyl-1H,2H,3H-pyrido[2,3-b][1,4]oxazin-7-yl}-5H,6H,7H,8H-pyrido[3,4-d]pyrimidin-2-yl)-N1-[2-(morpholin-4-yl)ethyl]benzene-1,4-diamine: To a stirred mixture of tert-butyl 7-{2-[(4-{[(tert-butoxy)carbonyl][2-(morpholin-4-yl)ethyl]amino}-3-methylphenyl) amino]-5H,6H,7H,8H-pyrido[3,4-d]pyrimidin-7-yl}-8-methyl-1H,2H,3H-pyrido[2,3-b][1,4]oxazine-1-carboxylate (110.00 mg; 0.12 mmol) in DCM (5.00 mL) and was added TFA (1.00 mL) at room temperature. After stirring for 3 h at room temperature, the mixture was concentrated under reduced pressure. The crude product was purified by Prep-HPLC (Column: X Bridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 25% B to 52% B in 8 min., 52% B; Wave Length: 254 nm; RTT (min.): 7) to afford 2-methyl-N4-(7-{8-methyl-1H,2H,3H-pyrido[2,3-b][1,4]oxazin-7-yl}-5H,6H,7H,8H-pyrido[3,4-d]pyrimidin-2-yl)-N1-[2-(morpholin-4-yl)ethyl]benzene-1,4-diamine (9.50 mg; 14.11%) as a yellow solid.

HPLC: 91.1% purity, RT=2.41 min. MS: m/z=517.3 [M+H]⁺. ¹H NMR (300 MHz, DMSO-d6): 9.02 (s, 1H), 8.23 (s, 1H), 7.43 (d, J=8.4 Hz, 1H), 7.28 (d, J=6.1 Hz, 2H), 6.58 (d, J=8.7 Hz, 1H), 5.57 (s, 1H), 4.20 (t, J=4.4 Hz, 2H), 3.89 (d, J=8 Hz, 8H), 3.49 (s, 2H), 3.34 (s, 6H), 3.11 (s, 2H), 2.75 (s, 2H), 2.11 (s, 3H), 2.04 (s, 3H).

### Example 24: Synthesis of Compound 24 N-(4-methanesulfonylphenyl)-7-{8-methyl-1H,2H,3H-pyrido[2,3-b][1,4]oxazin-7-yl}-5H,6H,7H,8H-pyrido[3,4-d]pyrimidin-2-amine

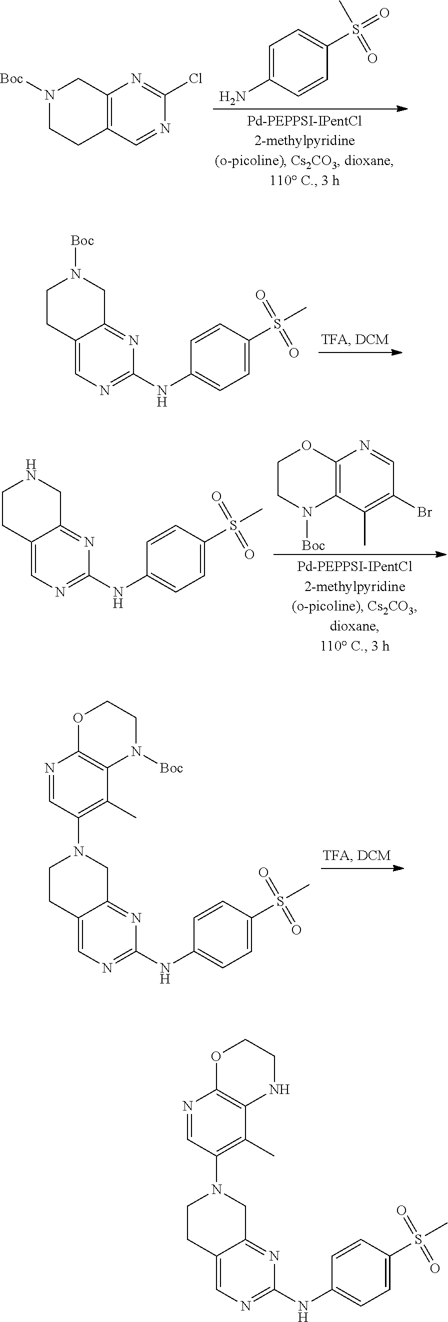

tert-butyl 2-[(4-methanesulfonylphenyl)amino]-5H,6H,7H,8H-pyrido[3,4-d]pyrimidine-7-carboxylate: To a stirred solution of tert-butyl 2-chloro-5H,6H,7H,8H-pyrido[3,4-d]pyrimidine-7-carboxylate (300.00 mg; 1.10 mmol) and in 1,4-dioxane (10.00 mL) was added Pd-PEPPSI-IPentCl 2-methylpyridine (o-picoline) (97.25 mg; 0.11 mmol) and Cs₂CO₃ (754.30 mg; 2.20 mmol). The resulting mixture was stirred for 3 h at 100° C. under nitrogen atmosphere. The resulting mixture was concentrated under reduced pressure. The residue was purified by silica gel column chromatography and eluted with PE/EtOAc (1:1) to afford tert-butyl 2-[(4-methanesulfonylphenyl)amino]-5H,6H,7H,8H-pyrido[3,4-d]pyrimidine-7-carboxylate (340.00 mg; 71.52%) as a yellow solid.

N-(4-methanesulfonylphenyl)-5H,6H,7H,8H-pyrido[3,4-d]pyrimidin-2-amine: To a stirred solution of tert-butyl 2-[(4-methanesulfonylphenyl)amino]-5H,6H,7H,8H-pyrido[3,4-d]pyrimidine-7-carboxylate (330.00 mg; 0.76 mmol) in DCM (10.00 mL) was added TFA (2.00 mL) in portions at room temperature. After stirring for 1 h at room temperature, the resulting mixture was concentrated under reduced pressure to afford N-(4-methanesulfonylphenyl)-5H,6H,7H,8H-pyrido[3,4-d]pyrimidin-2-amine (130.00 mg; 51.82%) as a yellow oil.

tert-butyl 7-{2-[(4-methanesulfonylphenyl)amino]-5H,6H,7H,8H-pyrido[3,4-d]pyrimidin-7-yl}-8-methyl-1H,2H,3H-pyrido[2,3-b][1,4]oxazine-1-carboxylate: To a stirred solution of N-(4-methanesulfonylphenyl)-5H,6H,7H,8H-pyrido[3,4-d]pyrimidin-2-amine (130.00 mg; 0.40 mmol) and tert-butyl 7-bromo-8-methyl-1H,2H,3H-pyrido[2,3-b][1,4]oxazine-1-carboxylate (136.96 mg; 0.40 mmol) in 1,4-dioxane (10.00 mL) was added Pd-PEPPSI-IPentCl 2-methylpyridine (o-picoline) (34.99 mg; 0.04 mmol) and Cs₂CO₃ (271.37 mg; 0.79 mmol). The resulting mixture was stirred for 3 h at 100° C. under nitrogen atmosphere. The resulting mixture was concentrated under reduced pressure. The residue was purified by silica gel column chromatography and eluted with PE/EtOAc (1:1) to afford tert-butyl 7-{2-[(4-methanesulfonylphenyl)amino]-5H,6H,7H,8H-pyrido[3,4-d]pyrimidin-7-yl}-8-methyl-1H,2H,3H-pyrido[2,3-b][1,4]oxazine-1-carboxylate (57.00 mg; 25.32%) as a yellow oil.

N-(4-methanesulfonylphenyl)-7-{8-methyl-1H,2H,3H-pyrido[2,3-b][1,4]oxazin-7-yl}-5H,6H,7H,8H-pyrido[3,4-d]pyrimidin-2-amine: To a stirred solution of tert-butyl 7-{2-[(4-methanesulfonylphenyl)amino]-5H,6H,7H,8H-pyrido[3,4-d]pyrimidin-7-yl}-8-methyl-1H,2H,3H-pyrido[2,3-b][1,4]oxazine-1-carboxylate (57.00 mg; 0.10 mmol) in DCM (5.00 mL) was added TFA (1.00 mL) in portions at room temperature. After stirring for 1 h at room temperature, the resulting mixture was concentrated under reduced pressure. The crude product was purified by Prep-HPLC with the following conditions: Column: XBridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 22% B to 52% B in 8 min., 52% B; Wave Length: 254 nm; RT1 (min.): 7; Number of Runs: 2, to afford N-(4-methanesulfonylphenyl)-7-{8-methyl-1H,2H,3H-pyrido[2,3-b][1,4]oxazin-7-yl}-5H,6H,7H,8H-pyrido[3,4-d]pyrimidin-2-amine (18.00 mg; 38.91%) as a yellow solid.

HPLC: 97.9% purity, RT=3.25 min. MS: m/z=453.1[M+H]⁺. ¹H NMR (300 MHz, DMSO-d6): 10.14 (s, 1H), 8.44 (s, 1H), 8.06-7.96 (m, 2H), 7.84-7.75 (m, 2H), 7.30 (s, 1H), 5.54 (s, 1H), 4.20 (d, J=5.0 Hz, 2H), 4.02 (s, 2H), 3.17 (d, J=5.4 Hz, 2H), 3.14 (s, 5H), 2.83 (d, J=6.0 Hz, 2H), 2.05 (s, 3H).

### Example 25: Synthesis of Compound 25 N-[4-(methanesulfonylmethoxy)phenyl]-7-{8-methyl-1H,2H,3H-pyrido[2,3-b][1,4]oxazin-7-yl}-5H,6H,7H,8H-pyrido[3,4-d]pyrimidin-2-amine

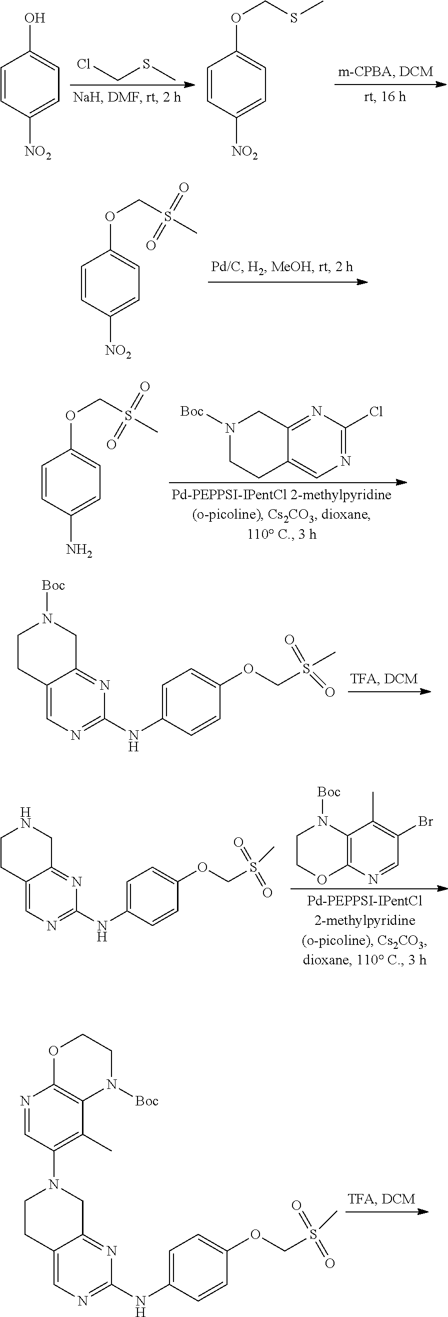

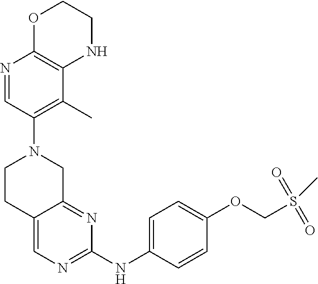 [SMILES: Cc1c(N2CCc3cnc(Nc4ccc(OCS(C)(=O)=O)cc4)nc3C2)cnc2c1NCCO2]

1-[(methylsulfanyl) methoxy]-4-nitrobenzene: To a stirred solution of 4-nitrophenol (1.00 g; 6.83 mmol) in DMF (15.00 mL) was added sodium hydride (683.00 mg; 17.08 mmol) at 0° C. under nitrogen atmosphere. The resulting mixture was stirred for 10 min. at 0° C. under nitrogen atmosphere. After 10 min., chloro (methylsulfanyl) methane (1041.00 mg; 10.24 mmol) was added to the above solution 0° C. under nitrogen atmosphere. Then the resulting mixture was stirred for additional 2 h at room temperature. The reaction was quenched by the addition of H₂O at room temperature and extracted by EA. The combined organic layers were washed with brine and dried over anhydrous Na₂SO₄. After filtration, the filtrate was concentrated under reduced pressure. The residue was purified by silica gel column chromatography and eluted with PE/EA (5:1) to afford 1-[(methylsulfanyl) methoxy]-4-nitrobenzene (500.00 mg; 35.93%) as a yellow solid.

1-(methanesulfonylmethoxy)-4-nitrobenzene: To a stirred solution of 1-[(methylsulfanyl)methoxy]-4-nitrobenzene (570.00 mg; 2.80 mmol) in DCM (15.00 mL) was added m-CPBA (2541.00 mg; 13.99 mmol) in portions at room temperature. Then the resulting mixture was stirred for additional 16 h at room temperature. The resulting mixture was concentrated under reduced pressure. The residue was purified by silica gel column chromatography and eluted with PE/EA (1:1) to afford 1-(methanesulfonylmethoxy)-4-nitrobenzene (480.00 mg; 73.71%) as a white solid.

4-(methanesulfonylmethoxy)aniline: To a solution of 1-(methanesulfonylmethoxy)-4-nitrobenzene (60.00 mg; 0.25 mmol) in MeOH (3.00 mL) was added Palladium on activated carbon (27.00 mg; 0.03 mmol) in a pressure tank. The mixture was hydrogenated at room temperature for 2 h under hydrogen atmosphere using a hydrogen balloon. The resulting mixture was filtered and the filter cake was washed with 30 mL of EA. The filtrate was concentrated under reduced pressure to afford 4-(methanesulfonylmethoxy) aniline (50.00 mg; 88.19%) as a yellow oil.

tert-butyl 2-{[4-(methanesulfonylmethoxy)phenyl]amino}-5H,6H,7H,8H-pyrido[3,4-d]pyrimidine-7-carboxylate: To a solution of tert-butyl 2-chloro-5H,6H,7H,8H-pyrido[3,4-d]pyrimidine-7-carboxylate (350.00 mg; 1.30 mmol) and 4-(methanesulfonylmethoxy)aniline (299.00 mg; 1.30 mmol) in 1,4-dioxane (15.00 mL) were added Cs₂CO₃ (890.00 mg; 2.59 mmol) and Pd-PEPPSI-IPentCl 2-methylpyridine (o-picoline) (57.00 mg; 0.06 mmol). The resulting mixture was stirred for 3 h at 100° C. under nitrogen atmosphere. The resulting mixture was concentrated under reduced pressure. The residue was purified by silica gel column chromatography and eluted with PE/EA (6:1) to afford tert-butyl 2-{[4-(methanesulfonylmethoxy)phenyl]amino}-5H,6H,7H,8H-pyrido[3,4-d]pyrimidine-7-carboxylate (300.00 mg; 52.94%) as a yellow solid.

N-[4-(methanesulfonylmethoxy)phenyl]-5H,6H,7H,8H-pyrido[3,4-d]pyrimidin-2-amine: To a stirred solution of tert-butyl 2-{[4-(methanesulfonylmethoxy)phenyl]amino}-5H,6H,7H,8H-pyrido[3,4-d]pyrimidine-7-carboxylate (290.00 mg; 0.66 mmol) and DCM (5.00 mL) was added TFA (1.00 mL) dropwise at room temperature. The resulting mixture was stirred for 1 h at room temperature. The mixture was basified to pH=7 with saturated NaHCO₃ (aq.) and extracted by EA. The combined organic layers were washed with brine and dried over anhydrous Na₂SO₄. After filtration, the filtrate was concentrated under reduced pressure to afford N-[4-(methanesulfonylmethoxy)phenyl]-5H,6H,7H,8H-pyrido[3,4-d]pyrimidin-2-amine (300.00 mg; 133.66%) as a yellow solid.

tert-butyl 7-(2-{[4-(methanesulfonylmethoxy)phenyl]amino}-5H,6H,7H,8H-pyrido[3,4-d]pyrimidin-7-yl)-8-methyl-1H,2H,3H-pyrido[2,3-b][1,4]oxazine-1-carboxylate: To a solution of N-[4-(methanesulfonylmethoxy)phenyl]-5H,6H,7H,8H-pyrido[3,4-d]pyrimidin-2-amine (163.00 mg; 0.48 mmol) and tert-butyl 7-bromo-8-methyl-1H,2H,3H-pyrido[2,3-b][1,4]oxazine-1-carboxylate (170.00 mg; 0.48 mmol) in 1,4-dioxane (15.00 mL) were added Cs₂CO₃ (331.00 mg; 0.97 mmol) and Pd-PEPPSI-IPentCl 2-methylpyridine (o-picoline) (21.00 mg; 0.02 mmol). The resulting mixture was stirred for 3 h at 100° C. under nitrogen atmosphere. The resulting mixture was concentrated under reduced pressure. The residue was purified by silica gel column chromatography and eluted with EA:PE (10:1) to afford tert-butyl 7-(2-{[4-(methanesulfonylmethoxy)phenyl]amino}-5H,6H,7H,8H-pyrido[3,4-d]pyrimidin-7-yl)-8-methyl-1H,2H,3H-pyrido[2,3-b][1,4]oxazine-1-carboxylate (140.00 mg; 49.07%) as a yellow solid.

N-[4-(methanesulfonylmethoxy)phenyl]-7-{8-methyl-1H,2H,3H-pyrido[2,3-b][1,4]oxazin-7-yl}-5H,6H,7H,8H-pyrido[3,4-d]pyrimidin-2-amine: To a stirred solution of tert-butyl 7-(2-{[4-(methanesulfonylmethoxy)phenyl]amino}-5H,6H,7H,8H-pyrido[3,4-d]pyrimidin-7-yl)-8-methyl-1H,2H,3H-pyrido[2,3-b][1,4]oxazine-1-carboxylate (120.00 mg; 0.20 mmol) and DCM (5.00 mL) was added TFA (1.00 mL) dropwise at room temperature. The resulting mixture was stirred for 1 h at room temperature. The mixture was basified to pH=7 with saturated NaHCO₃ (aq.) and extracted by EA. The combined organic layers were washed with brine and dried over anhydrous Na₂SO₄. After filtration, the filtrate was concentrated under reduced pressure. The residue was purified by Prep-HPLC with the following condition: Column: XBridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 25% B to 55% B in 8 min., 55% B; Wave Length: 254 nm; RT1 (min.): 7, to afford N-[4-(methanesulfonylmethoxy)phenyl]-7-{8-methyl-1H,2H,3H-pyrido[2,3-b][1,4]oxazin-7-yl}-5H,6H,7H,8H-pyrido[3,4-d]pyrimidin-2-amine (21.80 mg; 22.15%) as a yellow solid.

HPLC: 99.47% purity, RT=3.24 min. MS: m/z=483.20[M+H]⁺. ¹H NMR (300 MHz, DMSO-d6) 9.36 (s, 1H), 8.23 (s, 1H), 7.73-7.49 (m, 2H), 7.21 (s, 1H), 7.06-6.88 (m, 2H), 5.45 (t, J=2.7 Hz, 1H), 5.17 (s, 2H), 4.25-4.00 (m, 2H), 3.87 (s, 2H), 3.23 (d, J=4.2 Hz, 2H), 3.04 (t, J=5.6 Hz, 2H), 2.97 (s, 3H), 2.71 (t, J=5.6 Hz, 2H), 1.97 (s, 3H).

### Example 26: Synthesis of Compound 26 N2-[2-(dimethylamino)ethyl]-N5-(6-{8-methyl-1H,2H,3H-pyrido[2,3-b][1,4]oxazin-7-yl}-5,6,7,8-tetrahydro-2,6-naphthyridin-3-yl)pyridine-2,5-diamine

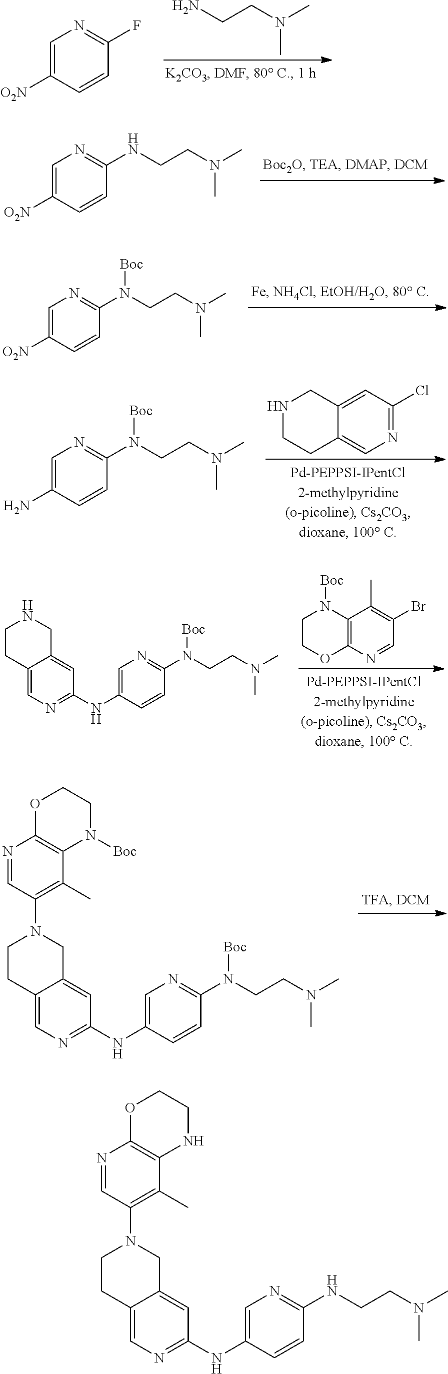

N-[2-(dimethylamino)ethyl]-5-nitropyridin-2-amine: To a stirred solution of 2-fluoro-5-nitropyridine (1.00 g; 6.69 mmol) and K₂CO₃ (2.91 g; 20.06 mmol) in DMF (10.00 mL) was added (2-aminoethyl)dimethylamine (0.93 g; 10.03 mmol) dropwise at room temperature. The resulting mixture was stirred for 2 h at 80° C. under N₂ atmosphere. The resulting mixture was filtered and the filter cake was washed with ACN. The filtrate was concentrated under reduced pressure. The residue was purified by reverse flash chromatography with the following conditions: column, C18 silica gel; mobile phase, MeCN in water (0.05% NH₄HCO₃), 100% to 100% gradient in 30 min.; detector, UV 254 nm, to afford N-[2-(dimethylamino)ethyl]-5-nitropyridin-2-amine (1.30 g; 92.1%) as a green solid.

tert-butyl N-[2-(dimethylamino)ethyl]-N-(5-nitropyridin-2-yl)carbamate: To a stirred solution of N-[2-(dimethylamino)ethyl]-5-nitropyridin-2-amine (1.28 g; 6.07 mmol) in DCM (3.00 mL) were added DMAP (0.14 g; 1.21 mmol), TEA (1.94 g; 18.20 mmol) and Boc₂O (4.01 g; 18.20 mmol) at 0° C. The resulting mixture was stirred for 1 h at room temperature under N₂ atmosphere. The residue was purified by silica gel column chromatography and eluted with DCM:MeOH (10:1) to afford tert-butyl N-[2-(dimethylamino)ethyl]-N-(5-nitropyridin-2-yl)carbamate (1.70 g; 89.4%.) as a yellow oil.

tert-butyl N-(5-aminopyridin-2-yl)-N-[2-(dimethylamino) ethyl]carbamate: To a stirred solution of tert-butyl N-[2-(dimethylamino)ethyl]-N-(5-nitropyridin-2-yl)carbamate (1.48 g; 4.72 mmol) in EtOH (15.00 mL) and water (5.00 mL) were added Fe (1.39 g; 23.61 mmol) and NH₄Cl (1.59 g; 28.33 mmol) at room temperature. The resulting mixture was stirred for 3 h at 80° C. under H₂ atmosphere. The resulting mixture was filtered and the filter cake was washed with MeOH (3×3 mL). The filtrate was concentrated under reduced pressure. The residue was purified by silica gel column chromatography and eluted with DCM:MeOH (10:1) to afford tert-butyl N-(5-aminopyridin-2-yl)-N-[2-(dimethylamino)ethyl]carbamate (1.30 g; 94.8%.) as a yellow solid.

tert-butyl N-[2-(dimethylamino)ethyl]-N-{5-[(5,6,7,8-tetrahydro-2,6-naphthyridin-3-yl)amino]pyridin-2-yl}carbamate: To a stirred mixture of tert-butyl N-(5-aminopyridin-2-yl)-N-[2-(dimethylamino)ethyl]carbamate (500.00 mg; 1.72 mmol) and 7-chloro-1,2,3,4-tetrahydro-2,6-naphthyridine (357.00 mg; 2.07 mmol) in 1,4-dioxane (5.00 mL) were added EPhos Pd G₄ (166.00 mg; 0.17 mmol), Ephos (97.00 mg; 0.17 mmol) and Cs₂CO₃ (1771.00 mg; 5.16 mmol) in portions at room temperature. The resulting mixture was stirred for 1 h at 120° C. under N₂ atmosphere. The resulting mixture was filtered and the filter cake was washed with MeOH (3×2 mL). The filtrate was concentrated under reduced pressure. The residue was purified by reverse flash chromatography with the following conditions: column, C18 silica gel; mobile phase, ACN in water (0.05% NH₃HCO₃), 10% to 100% gradient in 30 min.; detector, UV 254 nm, to afford tert-butyl N-[2-(dimethylamino)ethyl]-N-{5-[(5,6,7,8-tetrahydro-2,6-naphthyridin-3-yl)amino]pyridin-2-yl}carbamate (380.00 mg; 51.8%) as a brown solid.

tert-butyl 7-{7-[(6-{[(tert-butoxy)carbonyl][2-(dimethylamino)ethyl]amino}pyridin-3-yl)amino]-1,2,3,4-tetrahydro-2,6-naphthyridin-2-yl}-8-methyl-1H,2H,3H-pyrido[2,3-b][1,4]oxazine-1-carboxylate: To a stirred mixture of tert-butyl N-[2-(dimethylamino)ethyl]-N-{5-[(5,6,7,8-tetrahydro-2,6-naphthyridin-3-yl)amino]pyridin-2-yl}carbamate (360.00 mg; 0.85 mmol) and tert-butyl 7-bromo-8-methyl-1H,2H,3H-pyrido[2,3-b][1,4]oxazine-1-carboxylate (339.00 mg; 1.02 mmol) in 1,4-dioxane (4.00 mL) were added Pd-PEPPSI-IPentCl 2-methylpyridine (o-picoline) (75.00 mg; 0.08 mmol) and Cs₂CO₃ (580.00 mg; 1.69 mmol) in portions at room temperature. The resulting mixture was stirred for overnight at 100° C. under N₂ atmosphere. The mixture was allowed to cool down to room temperature. The resulting mixture was extracted with EtOAc (3×50 mL). The combined organic layers were washed with brine and dried over anhydrous NaSO₄. After filtration, the filtrate was concentrated under reduced pressure. The residue was purified by silica gel column chromatography and eluted with DCM:MeOH (10:1) to afford tert-butyl 7-{7-[(6-{[(tert-butoxy)carbonyl][2-(dimethylamino)ethyl]amino}pyridin-3-yl)amino]-1,2,3,4-tetrahydro-2,6-naphthyridin-2-yl}-8-methyl-1H,2H,3H-pyrido[2,3-b][1,4]oxazine-1-carboxylate (250.00 mg; 36.1%) as a yellow solid.

N2-[2-(dimethylamino)ethyl]-N5-(6-{8-methyl-1H,2H,3H-pyrido[2,3-b][1,4]oxazin-7-yl}-5,6,7,8-tetrahydro-2,6-naphthyridin-3-yl) pyridine-2,5-diamine: To a stirred mixture of tert-butyl 7-{7-[(6-{[(tert-butoxy)carbonyl][2-(dimethylamino)ethyl]amino}pyridin-3-yl)amino]-1,2,3,4-tetrahydro-2,6-naphthyridin-2-yl}-8-methyl-1H,2H,3H-pyrido[2,3-b][1,4]oxazine-1-carboxylate (230.00 mg; 0.28 mmol) in DCM (3.00 mL) was added TFA (1.00 mL) dropwise at 0° C. The resulting mixture was stirred for 1 h at room temperature under N₂ atmosphere. The resulting mixture was concentrated under vacuum. The crude product was purified by Prep-HPLC with the following conditions (Column: XBridge Shield RP18 OBD Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 22% to 50% in 9 min.; Wave Length: 254 nm; RT1 (min.): 7) to afford N2-[2-(dimethylamino)ethyl]-N5-(6-{8-methyl-1H,2H,3H-pyrido[2,3-b][1,4]oxazin-7-yl}-5,6,7,8-tetrahydro-2,6-naphthyridin-3-yl)pyridine-2,5-diamine (49.00 mg; 37.4%) as a yellow solid.

HPLC: 98.89% purity, RT=2.51 min. MS: m/z=461.30[M+H]⁺. ¹H NMR (400 MHz, DMSO-d6) 8.24 (s, 1H), 8.08 (d, J=2.7 Hz, 1H), 7.87 (s, 1H), 7.53 (dd, J=8.8, 2.7 Hz, 1H), 7.24 (s, 1H), 6.47 (d, J=8.9 Hz, 1H), 6.37 (s, 1H), 5.96 (t, J=5.6 Hz, 1H), 5.49 (s, 1H), 4.18 (t, J=4.4 Hz, 2H), 3.93 (s, 2H), 3.29 (d, J=5.9 Hz, 4H), 3.06 (s, 2H), 2.77 (s, 2H), 2.39 (t, J=6.6 Hz, 2H), 2.17 (s, 6H), 2.02 (s, 3H).

### Example 27: Synthesis of Compound 27 2-methyl-4-[(6-{8-methyl-1H,2H,3H-pyrido[2,3-b][1,4]oxazin-7-yl}-5,6,7,8-tetrahydro-2,6-naphthyridin-3-yl)amino]-N-[2-(morpholin-4-yl)ethyl]benzamide

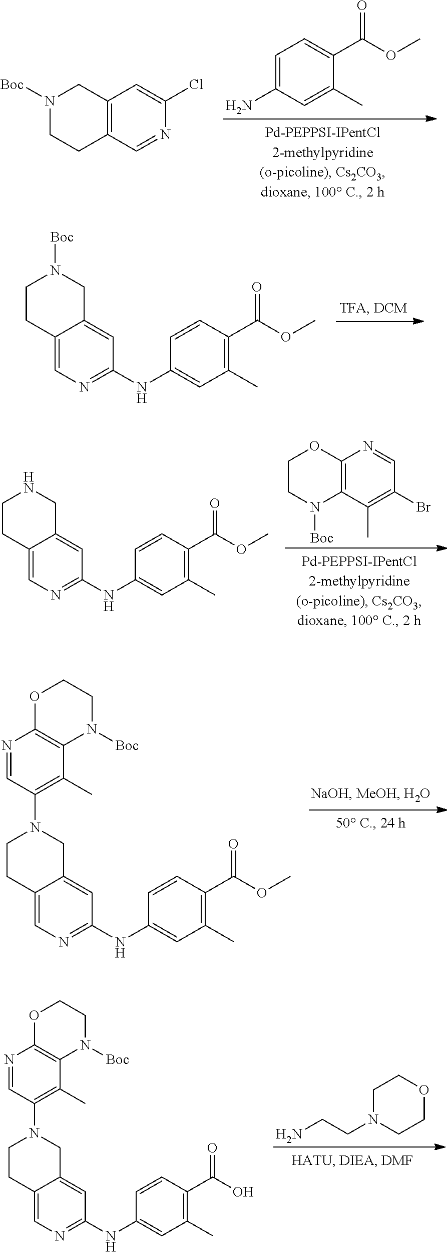

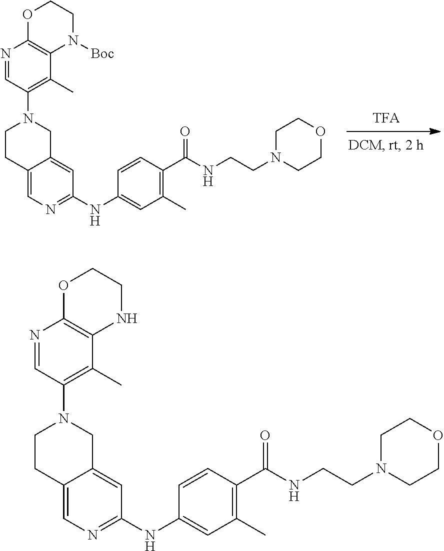 [SMILES: Cc1cc(Nc2cc3c(cn2)CCN(c2cnc4c(c2C)N(C(=O)OC(C)(C)C)CCO4)C3)ccc1C(=O)NCCN1CCOCC1 ; Cc1cc(Nc2cc3c(cn2)CCN(c2cnc4c(c2C)NCCO4)C3)ccc1C(=O)NCCN1CCOCC1]

tert-butyl 7-{[4-(methoxycarbonyl)-3-methylphenyl]amino}-1,2,3,4-tetrahydro-2,6-naphthyridine-2-carboxylate: To a stirred mixture of tert-butyl 7-chloro-1,2,3,4-tetrahydro-2,6-naphthyridine-2-carboxylate (400.00 mg; 1.21 mmol) and methyl 4-amino-2-methylbenzoate (400.28 mg; 2.42 mmol) in 1,4-dioxane (12.00 mL) were added Pd-PEPPSI-IPentCl 2-methylpyridine (o-picoline) (107.15 mg; 0.12 mmol) and Cs₂CO₃ (831.07 mg; 2.42 mmol) at room temperature. The mixture was stirred for 2 h at 100° C. The resulting mixture was concentrated under reduced pressure and purified by silica gel column chromatography and eluted with PE/EtOAc (1:1) to afford tert-butyl 7-{[4-(methoxycarbonyl)-3-methylphenyl]amino}-1,2,3,4-tetrahydro-2,6-naphthyridine-2-carboxylate (450.00 mg; 91.9%) as a yellow solid.

methyl 2-methyl-4-[(5,6,7,8-tetrahydro-2,6-naphthyridin-3-yl)amino]benzoate: To a stirred mixture of tert-butyl 7-{[4-(methoxycarbonyl)-3-methylphenyl]amino}-1,2,3,4-tetrahydro-2,6-naphthyridine-2-carboxylate (530.00 mg; 1.18 mmol) and TFA (3.00 mL) in DCM (15.00 mL) at room temperature. The mixture was stirred for 1 h at room temperature. The mixture was basified to pH=9 with sat. NaHCO₃ (aq.). The resulting mixture was quenched by ice/water and extracted with EtOAc. The combined organic layers were washed with brine and dried over anhydrous Na₂SO₄. After filtration, the filtrate was concentrated under reduced pressure. This resulted in methyl 2-methyl-4-[(5,6,7,8-tetrahydro-2,6-naphthyridin-3-yl)amino]benzoate (420.00 mg; crude product) as a yellow solid.

tert-butyl 7-(7-{[4-(methoxycarbonyl)-3-methylphenyl]amino}-1,2,3,4-tetrahydro-2,6-naphthyridin-2-yl)-8-methyl-1H,2H,3H-pyrido[2,3-b][1,4]oxazine-1-carboxylate: To a stirred mixture of methyl 2-methyl-4-[(5,6,7,8-tetrahydro-2,6-naphthyridin-3-yl)amino]benzoate (410.00 mg; 1.12 mmol) and tert-butyl 7-bromo-8-methyl-1H,2H,3H-pyrido[2,3-b][1,4]oxazine-1-carboxylate (738.95 mg; 2.24 mmol) in 1,4-dioxane (10.00 mL) were added Pd-PEPPSI-IPentCl 2-methylpyridine (o-picoline) (99.26 mg; 0.11 mmol) and Cs₂CO₃ (769.88 mg; 2.24 mmol) at room temperature. The mixture was stirred for 2 h at 100° C. The resulting mixture was concentrated under reduced pressure and purified by silica gel column chromatography and eluted with PE/EtOAc (1:1) to afford tert-butyl 7-(7-{[4-(methoxycarbonyl)-3-methylphenyl]amino}-1,2,3,4-tetrahydro-2,6-naphthyridin-2-yl)-8-methyl-1H,2H,3H-pyrido[2,3-b][1,4]oxazine-1-carboxylate (270.00 mg; 43.1%) as a yellow solid.

4-[(6-{1-[(tert-butoxy)carbonyl]-8-methyl-1H,2H,3H-pyrido[2,3-b][1,4]oxazin-7-yl}-5,6,7,8-tetrahydro-2,6-naphthyridin-3-yl)amino]-2-methylbenzoic acid: To a stirred mixture of tert-butyl 7-(7-{[4-(methoxycarbonyl)-3-methylphenyl]amino}-1,2,3,4-tetrahydro-2,6-naphthyridin-2-yl)-8-methyl-1H,2H,3H-pyrido[2,3-b][1,4]oxazine-1-carboxylate (260.00 mg; 0.34 mmol) in MeOH (6.00 mL) and H₂O (3.00 mL) was added NaOH (42.91 mg; 1.02 mmol) at room temperature. The mixture was stirred for 3 h at 50° C. The mixture was acidified to pH=7 with HCl (4N). The resulting mixture was quenched by ice/water and extracted with EtOAc. The combined organic layers were washed with brine and dried over anhydrous Na₂SO₄. After filtration, the filtrate was concentrated under reduced pressure. This resulted in 4-[(6-{1-[(tert-butoxy) carbonyl]-8-methyl-1H,2H,3H-pyrido[2,3-b][1,4]oxazin-7-yl}-5,6,7,8-tetrahydro-2,6-naphthyridin-3-yl)amino]-2-methylbenzoic acid (150.00 mg; crude product) as a yellow solid.

tert-butyl 8-methyl-7-{7-[(3-methyl-4-{[2-(morpholin-4-yl)ethyl]carbamoyl}phenyl)amino]-1,2,3,4-tetrahydro-2,6-naphthyridin-2-yl}-1H,2H,3H-pyrido[2,3-b][1,4]oxazine-1-carboxylate: To a stirred mixture of 4-[(6-{1-[(tert-butoxy)carbonyl]-8-methyl-1H,2H,3H-pyrido[2,3-b][1,4]oxazin-7-yl}-5,6,7,8-tetrahydro-2,6-naphthyridin-3-yl)amino]-2-methylbenzoic acid (180.00 mg; 0.28 mmol) and 2-(morpholin-4-yl)ethan-1-amine (71.76 mg; 0.55 mmol) in DMF (12.00 mL) were added HATU (330.94 mg; 0.83 mmol) and DIEA (74.97 mg; 0.55 mmol) at room temperature. The mixture was stirred for 1 h at room temperature. The resulting mixture was quenched by ice/water and extracted with EtOAc. The combined organic layers were washed with brine and dried over anhydrous Na₂SO₄. After filtration, the filtrate was concentrated under reduced pressure The resulting mixture was concentrated under reduced pressure and purified by silica gel column chromatography and eluted with CH₂Cl₂/MeOH (10:1) to afford tert-butyl 8-methyl-7-{7-[(3-methyl-4-{[2-(morpholin-4-yl)ethyl]carbamoyl}phenyl)amino]-1,2,3,4-tetrahydro-2,6-naphthyridin-2-yl}-1H,2H,3H-pyrido[2,3-b][1,4]oxazine-1-carboxylate (120.00 mg; 62.9%) as a brown solid.

2-methyl-4-[(6-{8-methyl-1H,2H,3H-pyrido[2,3-b][1,4]oxazin-7-yl}-5,6,7,8-tetrahydro-2,6-naphthyridin-3-yl)amino]-N-[2-(morpholin-4-yl)ethyl]benzamide: A mixture of tert-butyl 8-methyl-7-{7-[(3-methyl-4-{[2-(morpholin-4-yl)ethyl]carbamoyl}phenyl)amino]-1,2,3,4-tetrahydro-2,6-naphthyridin-2-yl}-1H,2H,3H-pyrido[2,3-b][1,4]oxazine-1-carboxylate (110.00 mg; 0.15 mmol) and TFA (1.00 mL) in DCM (5.00 mL) was stirred for 1 h at room temperature. The resulting mixture was concentrated under reduced pressure. The residue was purified by Prep-HPLC (Column: XBridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 22% B to 50% B in 9 min., 50% B; Wave Length: 254 nm; RT1 (min.): 7) to afford 2-methyl-4-[(6-{8-methyl-1H,2H,3H-pyrido[2,3-b][1,4]oxazin-7-yl}-5,6,7,8-tetrahydro-2,6-naphthyridin-3-yl)amino]-N-[2-(morpholin-4-yl)ethyl] benzamide (53.80 mg; 65.4%) as a yellow solid.

HPLC: 99.57% purity, RT=2.93 min. MS: m/z=544.25[M+H]⁺. ¹H NMR (300 MHz, DMSO-d6): 8.97 (s, 1H), 8.04 (s, 1H), 7.91 (t, J=5.7 Hz, 1H), 7.54 (dd, J=8.4, 2.2 Hz, 1H), 7.41 (d, J=2.2 Hz, 1H), 7.30-7.21 (m, 2H), 6.62 (s, 1H), 5.52 (s, 1H), 4.19 (t, J=4.4 Hz, 2H), 4.00 (s, 2H), 3.58 (t, J=4.6 Hz, 4H), 3.26 (s, 2H), 3.32 (s, 2H), 3.10 (s, 2H), 2.83 (s, 2H), 2.50-2.31 (m, 9H), 2.04 (s, 3H).

### Example 28: Synthesis of Compound 28 N2-[2-(dimethylamino)ethyl]-3-methyl-N5-(7-{8-methyl-1H,2H,3H-pyrido[2,3-b][1,4]oxazin-7-yl}-5H,6H,7H,8H-pyrido[3,4-d]pyrimidin-2-yl)pyridine-2,5-diamine

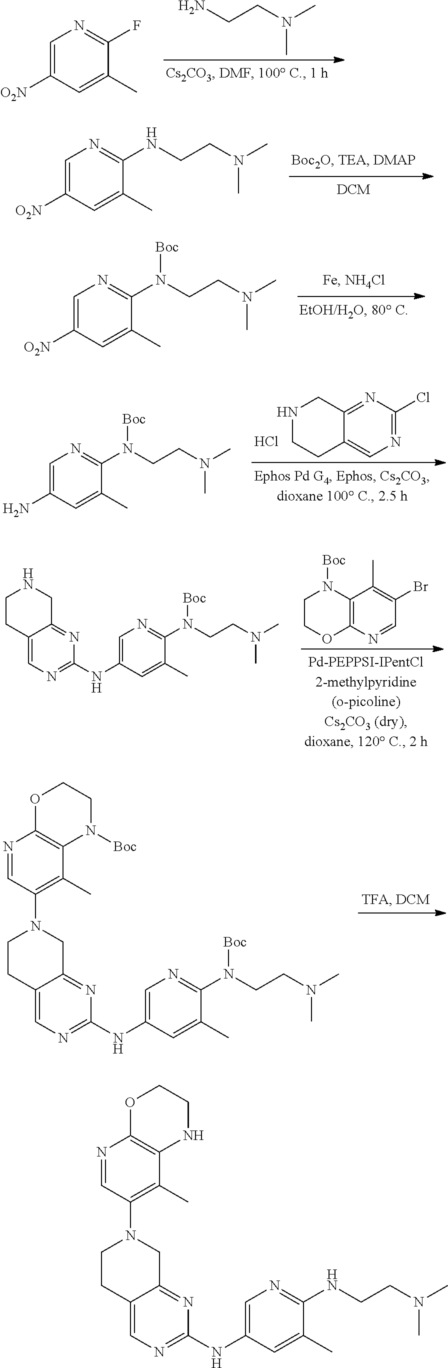

N-[2-(dimethylamino)ethyl]-3-methyl-5-nitropyridin-2-amine: To a stirred mixture of 2-fluoro-3-methyl-5-nitropyridine (1.00 g; 6.28 mmol) and (2-aminoethyl)dimethylamine (864.64 mg; 9.42 mmol) in DMF (10.00 mL) was added Cs₂CO₃ (6197.91 mg; 18.83 mmol) in portions at 25° C. under nitrogen atmosphere. The resulting mixture was stirred for 2 h at 100° C. under nitrogen atmosphere. The resulting mixture was extracted with EA (3×50 mL). The combined organic layers were washed with brine and dried over anhydrous MgSO₄. After filtration, the filtrate was concentrated under reduced pressure to afford N-[2-(dimethylamino)ethyl]-3-methyl-5-nitropyridin-2-amine (3.0 g; crude product) as an orange yellow solid.

tert-butyl N-[2-(dimethylamino)ethyl]-N-(3-methyl-5-nitropyridin-2-yl)carbamate: To a stirred mixture of N-[2-(dimethylamino)ethyl]-3-methyl-5-nitropyridin-2-amine (1.00 g; 4.46 mmol) and (Boc)₂O (3.14 mL; 13.38 mmol) in DCM (20.00 mL) was added TEA (1424.90 mg; 13.38 mmol) and DMAP (114.69 mg; 0.89 mmol) in portions at 25° C. under nitrogen atmosphere. The resulting mixture was stirred for 2 h at 25° C. under nitrogen atmosphere. The resulting mixture was extracted with EA (3×50 mL). The combined organic layers were washed with brine and dried over anhydrous MgSO₄. After filtration, the filtrate was concentrated under reduced pressure. The residue was purified by silica gel column chromatography and eluted with DCM:MEOH=95:5 to afford tert-butyl N-[2-(dimethylamino)ethyl]-N-(3-methyl-5-nitropyridin-2-yl)carbamate (1.10 g; 72.95%) as an orange yellow solid.

tert-butyl N-(5-amino-3-methylpyridin-2-yl)-N-[2-(dimethylamino)ethyl]carbamate: To a stirred mixture of tert-butyl N-[2-(dimethylamino)ethyl]-N-(3-methyl-5-nitropyridin-2-yl)carbamate (1.00 g; 2.96 mmol) and NH₄Cl (806.94 mg; 14.78 mmol) in EtOH (12.00 mL) and water (8.00 mL) was added Fe (733.93 mg; 11.83 mmol) in portions at 25° C. under nitrogen atmosphere. The resulting mixture was stirred for 2 h at 80° C. under nitrogen atmosphere. The resulting mixture was extracted with EA (3×50 mL). The combined organic layers were washed with brine and dried over anhydrous MgSO₄. After filtration, the filtrate was concentrated under reduced pressure. The residue was purified by silica gel column chromatography and eluted with DCM:MEOH=83:17 to afford tert-butyl N-(5-amino-3-methylpyridin-2-yl)-N-[2-(dimethylamino)ethyl]carbamate (915 mg; 97.75%) as an orange yellow solid.

tert-butyl N-[2-(dimethylamino)ethyl]-N-[3-methyl-5-({5H,6H,7H,8H-pyrido[3,4-d]pyrimidin-2-yl}amino)pyridin-2-yl]carbamate: To a stirred solution of 2-chloro-5H,6H,7H,8H-pyrido[3,4-d]pyrimidine hydrochloride (400.00 mg; 1.94 mmol) in 1,4-dioxane (8.00 mL) was added cesium carbonate (2662.90 mg; 7.76 mmol) in portions at room temperature under N₂ atmosphere. The mixture was stirred for 20 min. at room temperature. To the above mixture was added tert-butyl N-(5-amino-3-methylpyridin-2-yl)-N-[2-(dimethylamino)ethyl]carbamate (601.51 mg; 1.94 mmol), Ephos (109.27 mg; 0.19 mmol) and EPhos Pd G₄ (187.68 mg; 0.19 mmol) at room temperature. The resulting mixture was stirred for 2 h at 100° C. under N₂ atmosphere. The resulting mixture was concentrated under reduced pressure. The residue was purified by silica gel column and eluted with EA to afford tert-butyl N-[2-(dimethylamino)ethyl]-N-[3-methyl-5-({5H,6H,7H,8H-pyrido[3,4-d]pyrimidin-2-yl}amino)pyridin-2-yl]carbamate (280.00 mg; 25.68%) as a yellow solid.

tert-butyl 7-{2-[(6-{[(tert-butoxy)carbonyl][2-(dimethylamino)ethyl]amino}-5-methylpyridin-3-yl)amino]-5H,6H,7H,8H-pyrido[3,4-d]pyrimidin-7-yl}-8-methyl-1H,2H,3H-pyrido[2,3-b][1,4]oxazine-1-carboxylate: To a stirred solution of tert-butyl N-[2-(dimethylamino)ethyl]-N-[3-methyl-5-({5H,6H,7H,8H-pyrido[3,4-d]pyrimidin-2-yl}amino)pyridin-2-yl]carbamate (250.00 mg; 0.53 mmol) in 1,4-dioxane (5.00 mL) were added tert-butyl 7-bromo-8-methyl-1H,2H,3H-pyrido[2,3-b][1,4]oxazine-1-carboxylate (200.59 mg; 0.58 mmol), Pd-PEPPSI-IPentCl 2-methylpyridine (o-picoline) (46.54 mg; 0.05 mmol) and Cs₂CO₃ (541.47 mg; 1.58 mmol) in portions at room temperature under N₂ atmosphere. The resulting mixture was stirred for overnight at 120° C. under N₂ atmosphere. The resulting mixture was diluted with water (15 mL), extracted with EtOAc (3×10 mL). The combined organic layers were washed with brine (15 mL), dried over anhydrous Na₂SO₄. The resulting mixture was concentrated under reduced pressure. The residue was purified by silica gel column and eluted with 16% MeOH in DCM to afford tert-butyl 7-{2-[(6-{[(tert-butoxy)carbonyl][2-(dimethylamino)ethyl]amino}-5-methylpyridin-3-yl)amino]-5H,6H,7H,8H-pyrido[3,4-d]pyrimidin-7-yl}-8-methyl-1H,2H,3H-pyrido[2,3-b][1,4]oxazine-1-carboxylate (230.00 mg; 58.7%) as a brown orange solid.

N2-[2-(dimethylamino)ethyl]-3-methyl-N5-(7-{8-methyl-1H,2H,3H-pyrido[2,3-b][1,4]oxazin-7-yl}-5H,6H,7H,8H-pyrido[3,4-d]pyrimidin-2-yl)pyridine-2,5-diamine: To a stirred solution of tert-butyl 7-{2-[(6-{1[(tert-butoxy)carbonyl][2-(dimethylamino)ethyl]amino}-5-methylpyridin-3-yl)amino]-5H,6H,7H,8H-pyrido[3,4-d]pyrimidin-7-yl}-8-methyl-1H,2H,3H-pyrido[2,3-b][1,4]oxazine-1-carboxylate (210.00 mg; 0.30 mmol) in DCM (3.00 mL) was added TFA (0.60 mL) in portions at 0° C. under N₂ atmosphere. The resulting mixture was stirred for 1 h at room temperature under N₂ atmosphere. The resulting mixture was concentrated under reduced pressure. The residue was purified by Prep-HPLC with the following condition (Column: X Bridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 25% B to 55% B in 9 min., 55% B; Wave Length: 254 nm; RT1 (min.): 7) to afford N2-[2-(dimethylamino)ethyl]-3-methyl-N5-(7-{8-methyl-1H,2H,3H-pyrido[2,3-b][1,4]oxazin-7-yl}-5H,6H,7H,8H-pyrido[3,4-d]pyrimidin-2-yl)pyridine-2,5-diamine (23.80 mg; 16.6%) as a yellow solid.

HPLC: 97.93% purity, RT=2.05 min. MS: m/z=476.25 [M+H]⁺. ¹H NMR (400 MHz, DMSO-d6) 8.95 (s, 1H), 8.19 (d, J=17.8 Hz, 2H), 7.47 (s, 2H), 7.27 (s, 2H), 5.51 (s, 2H), 5.39 (s, 2H), 4.19 (s, 2H), 3.90 (s, 2H), 3.09 (s, 2H), 2.74 (s, 2H), 2.43 (s, 2H), 2.18 (s, 6H), 2.03 (s, 6H).

### Example 29: Synthesis of Compound 29 N-{4-[(7-{8-methyl-1H,2H,3H-pyrido[2,3-b][1,4]oxazin-7-yl}-5H,6H,7H,8H-pyrido[3,4-d]pyrimidin-2-yl)amino]-2-(trifluoromethyl)phenyl}-2-(morpholin-4-yl)acetamide

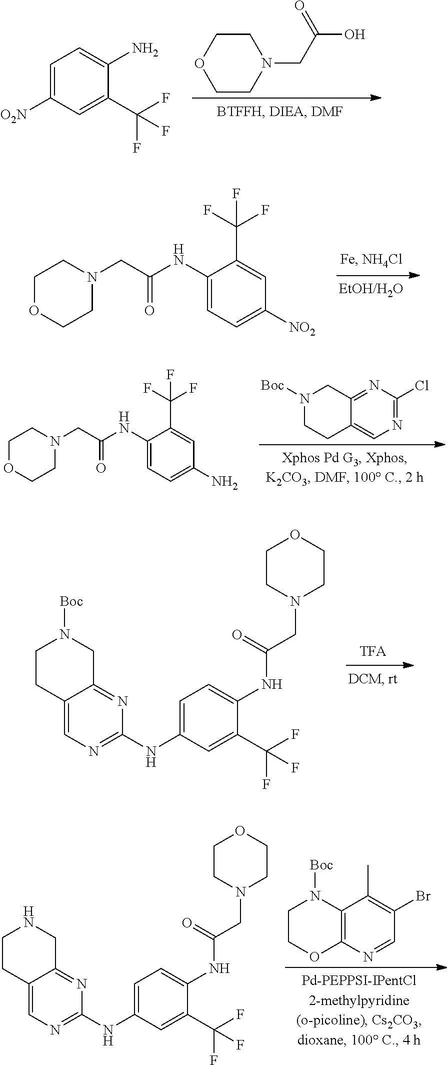

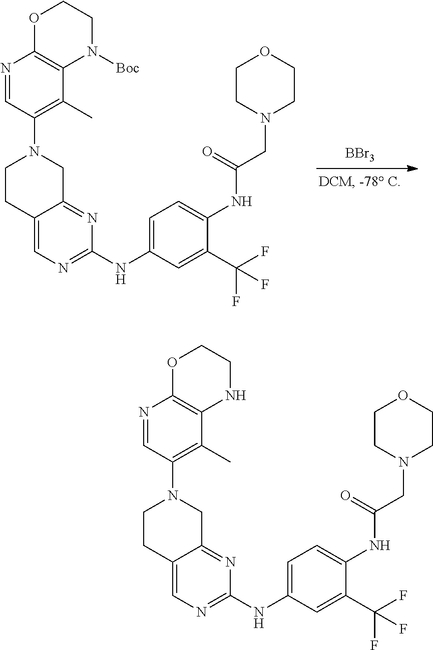

2-(morpholin-4-yl)-N-[4-nitro-2-(trifluoromethyl)phenyl]acetamide: A mixture of 2-(morpholin-4-yl)acetic acid (4358.77 mg; 28.53 mmol) and BTFFH (5932.26 mg; 17.83 mmol) in DMF (10.00 mL) was stirred for 30 min., then 4-nitro-2-(trifluoromethyl)aniline (1.50 g; 7.13 mmol) and DIEA (7.85 mL; 42.79 mmol) was added in portions at 25° C. under nitrogen atmosphere. The resulting mixture was stirred for 5 h at 80° C. under nitrogen atmosphere. The resulting mixture was extracted with EA (3×50 mL). The combined organic layers were washed with brine and dried over anhydrous MgSO₄. After filtration, the filtrate was concentrated under reduced pressure. The residue was purified by silica gel column chromatography and eluted with EA:PE=21:79 to afford 2-(morpholin-4-yl)-N-[4-nitro-2-(trifluoromethyl)phenyl]acetamide (4.20 g; 176.7%) as an off white solid.

N-[4-amino-2-(trifluoromethyl)phenyl]-2-(morpholin-4-yl)acetamide: To a stirred mixture of 2-(morpholin-4-yl)-N-[4-nitro-2-(trifluoromethyl)phenyl]acetamide (1.00 g; 3.00 mmol) and NH₄Cl (844.76 mg; 15.00 mmol) in EtOH (6.00 mL) and water (4.00 mL) was added Fe (707.52 mg; 12.00 mmol) in portions at 25° C. under nitrogen atmosphere. The resulting mixture was stirred for 4 h at 80° C. under nitrogen atmosphere. The resulting mixture was extracted with EA (3×50 mL). The combined organic layers were washed with brine and dried over anhydrous MgSO₄. After filtration, the filtrate was concentrated under reduced pressure. The residue was purified by silica gel column chromatography and eluted with DCM:MEOH=92:8 to afford N-[4-amino-2-(trifluoromethyl)phenyl]-2-(morpholin-4-yl)acetamide (1.60 g; 175.8%) as a yellow solid.

tert-butyl 2-({4-[2-(morpholin-4-yl) acetamido]-3-(trifluoromethyl) phenyl}amino)-5H,6H,7H,8H-pyrido[3,4-d]pyrimidine-7-carboxylate: To a stirred mixture of N-[4-amino-2-(trifluoromethyl)phenyl]-2-(morpholin-4-yl)acetamide (400.00 mg; 1.32 mmol) and tert-butyl 2-chloro-5H,6H,7H,8H-pyrido[3,4-d]pyrimidine-7-carboxylate (540.00 mg; 2.00 mmol) in DMF (10.00 mL) was added Xphos Pd G₃ (120.00 mg; 0.13 mmol), X-PHOS (60.00 mg; 0.12 mmol) and K₂CO₃ (560.00 mg; 3.85 mmol) in portions at 25° C. under nitrogen atmosphere. The resulting mixture was stirred for 2 h at 100° C. under nitrogen atmosphere. The resulting mixture was concentrated under vacuum. The residue was purified by silica gel column chromatography and eluted with (PE:EtOAc=7:3) to afford tert-butyl 2-({4-[2-(morpholin-4-yl)acetamido]-3-(trifluoromethyl)phenyl}amino)-5H,6H,7H,8H-pyrido[3,4-d]pyrimidine-7-carboxylate (650.00 mg; 90.7%) as a yellow solid.

2-(morpholin-4-yl)-N-[4-({5H,6H,7H,8H-pyrido[3,4-d]pyrimidin-2-yl}amino)-2-(trifluoromethyl)phenyl]acetamide: A solution of tert-butyl 2-({4-[2-(morpholin-4-yl)acetamido]-3-(trifluoromethyl)phenyl}amino)-5H,6H,7H,8H-pyrido[3,4-d]pyrimidine-7-carboxylate (600.00 mg; 1.10 mmol) and TFA (2.00 mL) in DCM (6.00 mL) was stirred for 1 h at 30° C. under nitrogen atmosphere. The mixture was basified to pH 12 with NaOH (4N). The resulting mixture was extracted with EtOAc. The combined organic layers were washed with brine and dried over anhydrous Na₂SO₄. After filtration, the filtrate was concentrated under reduced pressure. This resulted in 2-(morpholin-4-yl)-N-[4-({5H,6H,7H,8H-pyrido[3,4-d]pyrimidin-2-yl}amino)-2-(trifluoromethyl)phenyl]acetamide (540.00 mg; 96.0%) as a yellow solid.

tert-butyl 8-methyl-7-[2-({4-[2-(morpholin-4-yl)acetamido]-3-(trifluoromethyl)phenyl}amino)-5H,6H,7H,8H-pyrido[3,4-d]pyrimidin-7-yl]-1H,2H,3H-pyrido[2,3-b][1,4]oxazine-1-carboxylate: A solution of 2-(morpholin-4-yl)-N-[4-({5H,6H,7H,8H-pyrido[3,4-d]pyrimidin-2-yl}amino)-2-(trifluoromethyl)phenyl]acetamide (300.00 mg; 0.59 mmol) and tert-butyl 7-bromo-8-methyl-1H,2H,3H-pyrido[2,3-b][1,4]oxazine-1-carboxylate (340.00 mg; 0.98 mmol) and Pd-PEPPSI-IPentCl 2-methylpyridine (o-picoline) (60.00 mg; 0.07 mmol) and Cs₂CO₃ (674.00 mg; 1.97 mmol) in 1,4-dioxane (4.00 mL) was stirred for 2 h at 100° C. under nitrogen atmosphere. The resulting mixture was concentrated under vacuum. The residue was purified by silica gel column chromatography and eluted with (PE:EtOAc=7:3) to afford tert-butyl 8-methyl-7-[2-({4-[2-(morpholin-4-yl)acetamido]-3-(trifluoromethyl)phenyl}amino)-5H,6H,7H,8H-pyrido[3,4-d]pyrimidin-7-yl]-1H,2H,3H-pyrido[2,3-b][1,4]oxazine-1-carboxylate (188.00 mg; 46.6%) as a yellow solid.

N-{4-[(7-{8-methyl-1H,2H,3H-pyrido[2,3-b][1,4]oxazin-7-yl}-5H,6H,7H,8H-pyrido[3,4-d]pyrimidin-2-yl)amino]-2-(trifluoromethyl)phenyl}-2-(morpholin-4-yl)acetamide: A solution of tert-butyl 8-methyl-7-[2-({4-[2-(morpholin-4-yl)acetamido]-3-(trifluoromethyl)phenyl}amino)-5H,6H,7H,8H-pyrido[3,4-d]pyrimidin-7-yl]-1H,2H,3H-pyrido[2,3-b][1,4]oxazine-1-carboxylate (178.00 mg; 0.24 mmol) and TFA (2.00 mL) in DCM (6.00 mL) was stirred for 2 h at room temperature under nitrogen atmosphere. The crude product was purified by Prep-HPLC with the following conditions: Column: XBridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 29% B to 59% B in 9 min., 59% B; Wave Length: 254 nm; RT1 (min.): 7. This resulted in N-{4-[(7-{8-methyl-1H,2H,3H-pyrido[2,3-b][1,4]oxazin-7-yl}-5H,6H,7H,8H-pyrido[3,4-d]pyrimidin-2-yl)amino]-2-(trifluoromethyl)phenyl}-2-(morpholin-4-yl)acetamide (40.00 mg; 27.2%) as an off-white solid.

HPLC: 95.03% purity, RT=2.85 min. MS: m/z=585.20 [M+H]⁺. ¹H NMR (400 MHz, DMSO-d6) 9.88 (s, 1H), 9.61 (s, 1H), 8.39 (s, 1H), 8.23 (d, J=2.4 Hz, 1H), 8.01 (d, J=9.2 Hz, 1H), 7.90 (s, 1H), 7.29 (s, 1H), 5.54 (s, 1H), 4.19 (t, J=4.3 Hz, 2H), 3.97 (s, 2H), 3.65 (s, 6H), 3.16-3.11 (m, 4H), 2.81 (s, 2H), 2.26 (s, 4H), 2.04 (s, 3H).

### Example 30: Synthesis of Compound 2-methyl-1-{5-[(7-{8-methyl-1H,2H,3H-pyrido[2,3-b][1,4]oxazin-7-yl}-5H,6H,7H,8H-pyrido[3,4-d]pyrimidin-2-yl)amino]-2,3-dihydro-1H-isoindol-2-yl}propan-2-ol

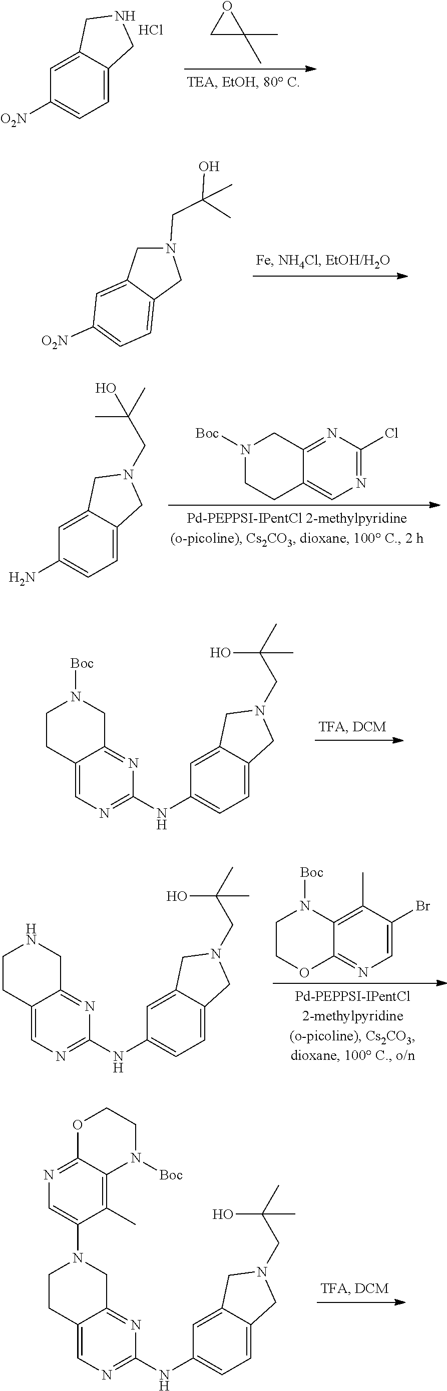

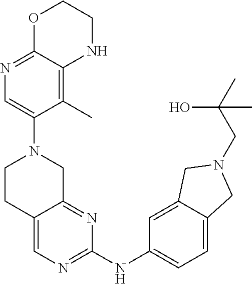 [SMILES: Cc1c(N2CCc3cnc(Nc4ccc5c(c4)CN(CC(C)(C)O)C5)nc3C2)cnc2c1NCCO2]

2-methyl-1-(5-nitro-2,3-dihydro-1H-isoindol-2-yl)propan-2-ol: To a stirred solution of 5-nitro-2,3-dihydro-1H-isoindole hydrochloride (320.00 mg; 1.52 mmol), TEA (0.89 mL; 6.06 mmol) in EtOH (15.00 mL) was added 2,2-dimethyloxirane (345.00 mg; 4.55 mmol) at room temperature. The resulting mixture was stirred for 16 h at 80° C. The reaction was cooled to room temperature. After filtration, the filtrate was concentrated under reduced pressure. This resulted in 2-methyl-1-(5-nitro-2,3-dihydro-1H-isoindol-2-yl)propan-2-ol (350.00 mg; 97.8%) as a yellow solid.

1-(5-amino-2,3-dihydro-1H-isoindol-2-yl)-2-methylpropan-2-ol: To a stirred mixture of 2-methyl-1-(5-nitro-2,3-dihydro-1H-isoindol-2-yl)propan-2-ol (350.00 mg; 1.48 mmol), NH₄Cl (404.00 mg; 7.40 mmol) in EtOH (10.00 mL) and water (5.00 mL) was added Fe (422.00 mg; 7.40 mmol) at room temperature. The resulting mixture was stirred for 3 h at 80° C. The reaction was cooled to room temperature. After filtration, the filtrate was concentrated under reduced pressure. The residue was purified by silica gel column chromatography and eluted with DCM/MeOH (3:1) to afford 1-(5-amino-2,3-dihydro-1H-isoindol-2-yl)-2-methylpropan-2-ol (300.00 mg; 90.5%) as a yellow brown solid.

tert-butyl 2-{[2-(2-hydroxy-2-methylpropyl)-2,3-dihydro-1H-isoindol-5-yl]amino}-5H,6H,7H,8H-pyrido[3,4-d]pyrimidine-7-carboxylate: To a stirred solution of 1-(5-amino-2,3-dihydro-1H-isoindol-2-yl)-2-methylpropan-2-ol (300.00 mg; 1.26 mmol) and tert-butyl 2-chloro-5H,6H,7H,8H-pyrido[3,4-d]pyrimidine-7-carboxylate (428.00 mg; 1.51 mmol) in 1,4-dioxane (15.00 mL) was added Cs₂CO₃ (862.00 mg; 2.51 mmol) and Pd-PEPPSI-IPentCl 2-methylpyridine (o-picoline) (111.00 mg; 0.13 mmol) in portions at room temperature under N₂ atmosphere. The resulting mixture was stirred for 16 h at 100° C. under N₂ atmosphere. The reaction was quenched with water at room temperature. The resulting mixture was extracted with EtOAc (3×50 mL). The combined organic layers were washed with brine (1×50 mL) and dried over anhydrous Na₂SO₄. After filtration, the filtrate was concentrated under reduced pressure. The residue was purified by silica gel column chromatography and eluted with DCM/MeOH (4:1) to afford tert-butyl 2-{[2-(2-hydroxy-2-methylpropyl)-2,3-dihydro-1H-isoindol-5-yl]amino}-5H,6H,7H,8H-pyrido[3,4-d]pyrimidine-7-carboxylate (380.00 mg; 68.8%) as a brown yellow solid.

2-methyl-1-[5-({5H,6H,7H,8H-pyrido[3,4-d]pyrimidin-2-yl}amino)-2,3-dihydro-1H-isoindol-2-yl]propan-2-ol: To a stirred solution of tert-butyl 2-{[2-(2-hydroxy-2-methylpropyl)-2,3-dihydro-1H-isoindol-5-yl]amino}-5H,6H,7H,8H-pyrido[3,4-d]pyrimidine-7-carboxylate (380.00 mg; 0.86 mmol) in DCM (9.00 mL) was added TFA (3.00 mL) dropwise at room temperature. The resulting mixture was stirred for 3 h at room temperature. The filtrate was concentrated under reduced pressure. The residue was purified by C18 silica gel column chromatography and eluted with H₂O/MeCN (1:4) to afford 2-methyl-1-[5-({5H,6H,7H,8H-pyrido[3,4-d]pyrimidin-2-yl}amino)-2,3-dihydro-1H-isoindol-2-yl]propan-2-ol (320.00 mg; 90.9%) as a brown solid.

tert-butyl 7-(2-{[2-(2-hydroxy-2-methylpropyl)-2,3-dihydro-1H-isoindol-5-yl]amino}-5H,6H,7H,8H-pyrido[3,4-d]pyrimidin-7-yl)-8-methyl-1H,2H,3H-pyrido[2,3-b][1,4]oxazine-1-carboxylate: To a stirred solution of 2-methyl-1-[5-({5H,6H,7H,8H-pyrido[3,4-d]pyrimidin-2-yl}amino)-2,3-dihydro-1H-isoindol-2-yl]propan-2-ol (300.00 mg; 0.74 mmol) and tert-butyl 7-bromo-8-methyl-1H,2H,3H-pyrido[2,3-b][1,4]oxazine-1-carboxylate (306.00 mg; 0.88 mmol) in 1,4-dioxane (15.00 mL) was added Cs₂CO₃ (505.00 mg; 1.47 mmol) and Pd-PEPPSI-IPentCl 2-methylpyridine (o-picoline) (65.00 mg; 0.07 mmol) in portions at room temperature under N₂ atmosphere. The resulting mixture was stirred for 16 h at 100° C. under N₂ atmosphere. The reaction was quenched with water at room temperature. The resulting mixture was extracted with EtOAc (3×50 mL). The combined organic layers were washed with brine (1×50 mL) and dried over anhydrous Na₂SO₄. After filtration, the filtrate was concentrated under reduced pressure. The residue was purified by silica gel column chromatography and eluted with DCM/MeOH (4:1) to afford tert-butyl 7-(2-{[2-(2-hydroxy-2-methylpropyl)-2,3-dihydro-1H-isoindol-5-yl]amino}-5H,6H,7H,8H-pyrido[3,4-d]pyrimidin-7-yl)-8-methyl-1H,2H,3H-pyrido[2,3-b][1,4]oxazine-1-carboxylate (260.00 mg; 60.0%) as a yellow solid.

2-methyl-1-{5-[(7-{8-methyl-1H,2H,3H-pyrido[2,3-b][1,4]oxazin-7-yl}-5H,6H,7H,8H-pyrido[3,4-d]pyrimidin-2-yl)amino]-2,3-dihydro-1H-isoindol-2-yl}propan-2-ol: To a stirred solution of tert-butyl 7-(2-{[2-(2-hydroxy-2-methylpropyl)-2,3-dihydro-1H-isoindol-5-yl]amino}-5H,6H,7H,8H-pyrido[3,4-d]pyrimidin-7-yl)-8-methyl-1H,2H,3H-pyrido[2,3-b][1,4]oxazine-1-carboxylate (240.00 mg; 0.41 mmol) in DCM (9.00 mL) was added TFA (3.00 mL) dropwise at room temperature. The resulting mixture was stirred for 3 h at room temperature. The resulting mixture was concentrated under reduced pressure. The residue was purified by Prep-HPLC with the following condition: Column: Xselect CSH C18 OBD Column 30*150 mm, 5 μm, n; Mobile Phase A: water (0.1% FA), Mobile Phase B: ACN; Flow rate: 60 mL/min.; Gradient: 5% B to 23% B in 8 min., 23% B; Wavelength: 254/220 nm; RT1 (min.): 6.3, to afford 2-methyl-1-{5-[(7-{8-methyl-1H,2H,3H-pyrido[2,3-b][1,4]oxazin-7-yl}-5H,6H,7H,8H-pyrido[3,4-d]pyrimidin-2-yl)amino]-2,3-dihydro-1H-isoindol-2-yl}propan-2-ol (21.20 mg; 10.4%) as a yellow solid.

HPLC: 97.59% purity, RT=2.44 min. MS: m/z=488.30 [M+H]⁺. ¹H NMR (300 MHz, Methanol-d4) 8.22 (s, 1H), 7.60 (s, 1H), 7.42 (dd, J=8.2, 2.0 Hz, 1H), 7.27 (s, 1H), 7.11 (d, J=8.2 Hz, 1H), 4.35-4.26 (m, 2H), 4.07-3.95 (m, 6H), 3.46-3.37 (m, 2H), 3.19 (t, J=5.7 Hz, 2H), 2.84 (t, J=5.7 Hz, 2H), 2.75 (s, 2H), 2.14 (s, 3H), 1.26 (s, 6H).

### Example 31: Synthesis of Compound 31 2-(dimethylamino)-N-{5-[(6-{8-methyl-1H,2H,3H-pyrido[2,3-b][1,4]oxazin-7-yl}-5,6,7,8-tetrahydro-2,6-naphthyridin-3-yl)amino]pyridin-2-yl}acetamide

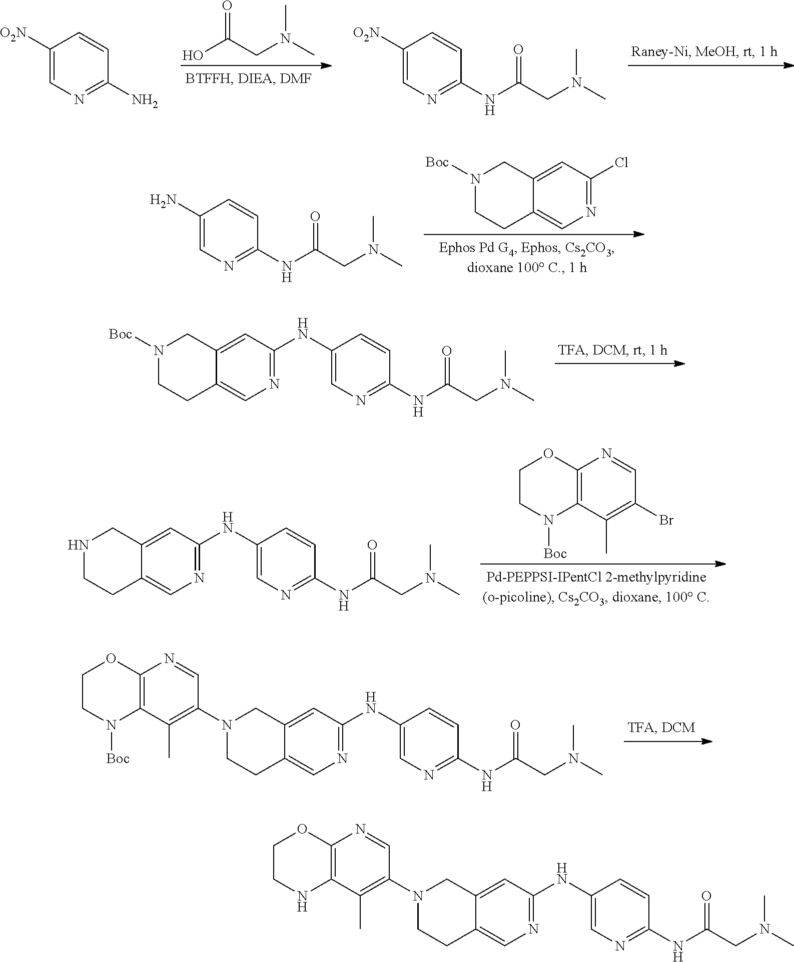

2-(dimethylamino)-N-(5-nitropyridin-2-yl)acetamide: To a stirred solution of 5-nitropyridin-2-amine (1.00 g; 6.97 mmol) in DMF (10.00 mL) were added BTFFH (3.48 g; 10.46 mmol), DIEA (4.74 g; 34.86 mmol) and 2-(dimethylamino) acetic acid (2.42 g; 20.92 mmol) at 0° C. under N₂ atmosphere. The resulting mixture was allowed to cool down to room temperature. The residue was purified by reverse flash chromatography with the following conditions: column, C18 silica gel; mobile phase, MeCN in water (0.05% NH₄HCO₃), 70% to 80% gradient in 10 min.; detector, UV 254 nm, to afford 2-(dimethylamino)-N-(5-nitropyridin-2-yl)acetamide (800.00 mg; 48.5%) as a yellow solid.

N-(5-aminopyridin-2-yl)-2-(dimethylamino)acetamide: To a solution of 2-(dimethylamino)-N-(5-nitropyridin-2-yl)acetamide (780.00 mg; 3.30 mmol) in MeOH (10.00 mL) mL was added Raney-Ni (500.00 mg; 5.54 mmol) at room temperature. The resulting mixture was stirred for 1 h at room temperature under hydrogen atmosphere. The resulting mixture was filtered and the filter cake was washed with MeOH (3×20 mL). The filtrate was concentrated under reduced pressure. This resulted in N-(5-aminopyridin-2-yl)-2-(dimethylamino) acetamide (600.00 mg; 92.6%) as a red oil.

tert-butyl 7-({6-[2-(dimethylamino)acetamido]pyridin-3-yl}amino)-1,2,3,4-tetrahydro-2,6-naphthyridine-2-carboxylate: To a stirred solution of N-(5-aminopyridin-2-yl)-2-(dimethylamino) acetamide (258.00 mg; 1.31 mmol) and tert-butyl 7-chloro-1,2,3,4-tetrahydro-2,6-naphthyridine-2-carboxylate (300.00 mg; 1.09 mmol) in 1,4-dioxane (5.00 mL) were added EPhos Pd G₄ (106.00 mg; 0.11 mmol), Cs₂CO₃ (1.08 g; 3.28 mmol) and Ephos (62.00 mg; 0.11 mmol) at room temperature under N₂ atmosphere. The resulting mixture was stirred for 1 h at 100° C. under N₂ atmosphere. The resulting mixture was allowed to cool down to room temperature. The resulting mixture was diluted with water. The resulting mixture was extracted with EtOAc (3×20 mL). The combined organic layers were washed with brine and dried over anhydrous Na₂SO₄. After filtration, the filtrate was concentrated under reduced pressure. The residue was purified by reverse flash chromatography with the following conditions: column, C18 silica gel; mobile phase, MeCN in water (0.05% NH₄HCO₃), 70% to 80% gradient in 10 min.; detector, UV 254 nm, to afford tert-butyl 7-({6-[2-(dimethylamino)acetamido]pyridin-3-yl}amino)-1,2,3,4-tetrahydro-2,6-naphthyridine-2-carboxylate (400.00 mg; 85.5%) as a brown solid.

2-(dimethylamino)-N-{5-[(5,6,7,8-tetrahydro-2,6-naphthyridin-3-yl)amino]pyridin-2-yl}acetamide: To a stirred solution of tert-butyl 7-({6-[2-(dimethylamino)acetamido]pyridin-3-yl}amino)-1,2,3,4-tetrahydro-2,6-naphthyridine-2-carboxylate (380.00 mg; 0.89 mmol) in DCM (3.00 mL) was added TFA (1.00 mL) dropwise at room temperature under N₂ atmosphere. The resulting mixture was stirred for 1 h at room temperature under N₂ atmosphere. The resulting mixture was concentrated under vacuum. The residue was purified by reverse flash chromatography with the following conditions: column, C18 silica gel; mobile phase, MeCN in water (0.05% NH₄HCO₃), 10% to 20% gradient in 30 min.; detector, UV 254 nm. This resulted in 2-(dimethylamino)-N-{5-[(5,6,7,8-tetrahydro-2,6-naphthyridin-3-yl)amino]pyridin-2-yl}acetamide (280.00 mg; 96.1%) as a light brown oil.

tert-butyl 7-[7-({6-[2-(dimethylamino)acetamido]pyridin-3-yl}amino)-1,2,3,4-tetrahydro-2,6-naphthyridin-2-yl]-8-methyl-1H,2H,3H-pyrido[2,3-b][1,4]oxazine-1-carboxylate: To a stirred solution of 2-(dimethylamino)-N-{5-[(5,6,7,8-tetrahydro-2,6-naphthyridin-3-yl)amino]pyridin-2-yl}acetamide (130.00 mg; 0.40 mmol) and tert-butyl 7-bromo-8-methyl-1H,2H,3H-pyrido[2,3-b][1,4]oxazine-1-carboxylate (157.00 mg; 0.48 mmol) in 1,4-dioxane (2.00 mL) were added Pd-PEPPSI-IPentCl 2-methylpyridine (o-picoline) (35.00 mg; 0.04 mmol) and Cs₂CO₃ (408.00 mg; 1.19 mmol) in portions at room temperature under N₂ atmosphere. The resulting mixture was stirred for 24 h at 120° C. under N₂ atmosphere. The resulting mixture was allowed to cool down to room temperature. The resulting mixture was filtered and the filter cake was washed with MeOH (3×5 mL). The filtrate was concentrated under reduced pressure. The crude product was purified by Prep-TLC (DCM:MeOH 20:1) to afford tert-butyl 7-[7-({6-[2-(dimethylamino)acetamido]pyridin-3-yl}amino)-1,2,3,4-tetrahydro-2,6-naphthyridin-2-yl]-8-methyl-1H,2H,3H-pyrido[2,3-b][1,4]oxazine-1-carboxylate (50.00 mg; 20.6%) as a yellow solid.

2-(dimethylamino)-N-{5-[(6-{8-methyl-1H,2H,3H-pyrido[2,3-b][1,4]oxazin-7-yl}-5,6,7,8-tetrahydro-2,6-naphthyridin-3-yl)amino]pyridin-2-yl}acetamide: To a stirred solution of tert-butyl 7-[7-({6-[2-(dimethylamino)acetamido]pyridin-3-yl}amino)-1,2,3,4-tetrahydro-2,6-naphthyridin-2-yl]-8-methyl-1H,2H,3H-pyrido[2,3-b][1,4]oxazine-1-carboxylate (50.00 mg; 0.08 mmol) in DCM (1.50 mL) was added TFA (0.50 mL) at 0° C. under N₂ atmosphere. The resulting mixture was stirred for 1 h at room temperature under N₂ atmosphere. The resulting mixture was concentrated under vacuum. The crude product was purified by Prep-HPLC with the following conditions (Column: XBridge Prep OBD C18 Column, 30*150 mm, 5 μm; Mobile Phase A: water (10 mmol/L NH₄HCO₃+0.05% NH₄OH), Mobile Phase B: MeOH-HPLC; Flow rate: 60 mL/min.; Gradient: 48% to 78% in 8 min.; Wave Length: 254 nm; RT1 (min.): 7) to afford 2-(dimethylamino)-N-{5-[(6-{8-methyl-1H,2H,3H-pyrido[2,3-b][1,4]oxazin-7-yl}-5,6,7,8-tetrahydro-2,6-naphthyridin-3-yl)amino]pyridin-2-yl}acetamide (13.00 mg; 33.3%) as a white solid.

HPLC: 99.35% purity, RT=4.63 min. MS: m/z=475.25 [M+H]⁺. ¹H NMR (300 MHz, Methanol-d4) 8.49 (dd, J=2.3, 1.2 Hz, 1H), 8.09-7.96 (m, 2H), 7.97 (s, 1H), 7.26 (s, 1H), 6.56 (s, 1H), 4.30 (t, J=4.4 Hz, 2H), 4.03 (s, 2H), 3.41 (t, J=4.4 Hz, 2H), 3.17 (d, J=5.9 Hz, 4H), 2.90 (t, J=5.7 Hz, 2H), 2.40 (s, 6H), 2.13 (s, 3H).

### Example 32: Synthesis of Compound 32 N2-[2-(dimethylamino)ethyl]-3-methyl-N5-(7-{8-methyl-1H,2H,3H-pyrido[2,3-b][1,4]oxazin-7-yl}-5H,6H,7H,8H-pyrido[3,4-d]pyrimidin-2-yl)pyridine-2,5-diamine

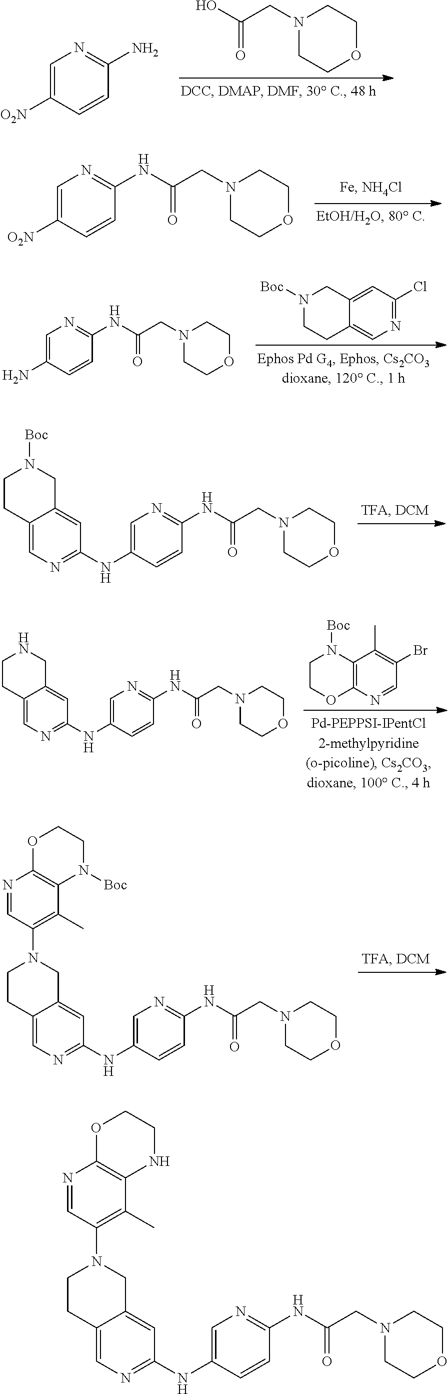

2-(morpholin-4-yl)-N-(5-nitropyridin-2-yl)acetamide: To a stirred solution of 2-(morpholin-4-yl)acetic acid (3.20 g; 20.92 mmol) in DMF (20.00 mL) were added DMAP (0.36 g; 2.79 mmol), 5-nitropyridin-2-amine (2.00 g; 13.95 mmol) and DCC (4.54 g; 20.92 mmol) in portions at 0° C. under N₂ atmosphere. The resulting mixture was stirred for 3 days at 30° C. under N₂ atmosphere. The resulting mixture was diluted with water (15 mL), extracted with EtOAc (3×10 mL). The combined organic layers were washed with brine (15 mL) and dried over anhydrous Na₂SO₄. The resulting mixture was concentrated under reduced pressure. The residue was purified by silica gel column and eluted with 90% EtOAc in PE to afford 2-(morpholin-4-yl)-N-(5-nitropyridin-2-yl) acetamide (2.55 g; 68.7%) as a light yellow solid.

N-(5-aminopyridin-2-yl)-2-(morpholin-4-yl) acetamide: To a stirred solution of 2-(morpholin-4-yl)-N-(5-nitropyridin-2-yl) acetamide (2.48 g; 8.38 mmol) in EtOH (15.00 mL) were added Fe (2.47 g; 41.92 mmol) and NH₄Cl (2.36 g; 41.92 mmol (dissolved in H₂O (10.00 mL)) in portions at room temperature under N₂ atmosphere. The resulting mixture was stirred for 1.5 h at 80° C. under N₂ atmosphere. The residue was purified by silica gel column and eluted with 35% DCM in MeOH to afford N-(5-aminopyridin-2-yl)-2-(morpholin-4-yl) acetamide (2.00 g; 99.0%) as a yellow oil.

tert-butyl 7-({6-[2-(morpholin-4-yl)acetamido]pyridin-3-yl}amino)-1,2,3,4-tetrahydro-2,6-naphthyridine-2-carboxylate: To a stirred solution of N-(5-aminopyridin-2-yl)-2-(morpholin-4-yl) acetamide (280.00 mg; 1.07 mmol) in 1,4-dioxane (5.00 mL) were added tert-butyl 7-chloro-1,2,3,4-tetrahydro-2,6-naphthyridine-2-carboxylate (331.89 mg; 1.17 mmol), EPhos Pd G₄ (103.13 mg; 0.11 mmol), Ephos (120.08 mg; 0.21 mmol) and Cesium Carbonate (1097.40 mg; 3.20 mmol) in portions at room temperature under N₂ atmosphere. The resulting mixture was stirred for 1 h at 120° C. under N₂ atmosphere. The resulting mixture was diluted with water (15 mL), extracted with EtOAc (3×10 mL). The combined organic layers were washed with brine (15 mL) and dried over anhydrous Na₂SO₄. The resulting mixture was concentrated under reduced pressure. The residue was purified by silica gel column and eluted with 7% MeOH in DCM to afford tert-butyl 7-({6-[2-(morpholin-4-yl)acetamido]pyridin-3-yl}amino)-1,2,3,4-tetrahydro-2,6-naphthyridine-2-carboxylate (600.00 mg; 100%) as a yellow solid.

2-(morpholin-4-yl)-N-{5-[(5,6,7,8-tetrahydro-2,6-naphthyridin-3-yl)amino]pyridin-2-yl}acetamide: To a stirred solution of tert-butyl 7-({6-[2-(morpholin-4-yl)acetamido]pyridin-3-yl}amino)-1,2,3,4-tetrahydro-2,6-naphthyridine-2-carboxylate (630.00 mg; 1.34 mmol) in DCM (5.00 mL) was added TFA (1.00 mL) in portions at 0° C. under N₂ atmosphere. The resulting mixture was stirred for 1 h at room temperature under N₂ atmosphere. The resulting mixture was adjusted pH=12 with NaOH (4N), then extracted with DCM (3×10 mL). The combined organic layers were washed with brine (15 mL) and dried over anhydrous Na₂SO₄. The resulting mixture was concentrated under reduced pressure to afford 2-(morpholin-4-yl)-N-{5-[(5,6,7,8-tetrahydro-2,6-naphthyridin-3-yl)amino]pyridin-2-yl}acetamide (550.00 mg; crude product) as a light yellow solid.

tert-butyl 8-methyl-7-[7-({6-[2-(morpholin-4-yl)acetamido]pyridin-3-yl}amino)-1,2,3,4-tetrahydro-2,6-naphthyridin-2-yl]-1H,2H,3H-pyrido[2,3-b][1,4]oxazine-1-carboxylate: To a stirred solution of 2-(morpholin-4-yl)-N-{5-[(5,6,7,8-tetrahydro-2,6-naphthyridin-3-yl)amino]pyridin-2-yl}acetamide (280.00 mg; 0.66 mmol) in 1,4-dioxane (5.00 mL) were added tert-butyl 7-bromo-8-methyl-1H,2H,3H-pyrido[2,3-b][1,4]oxazine-1-carboxylate (252.02 mg; 0.73 mmol), Pd-PEPPSI-IPentCl 2-methylpyridine (o-picoline) (58.47 mg; 0.07 mmol) and Cs₂CO₃ (680.29 mg; 1.98 mmol) in portions at room temperature under N₂ atmosphere. The resulting mixture was stirred for 1 h at 100° C. under N₂ atmosphere. The resulting mixture was diluted with water (15 mL), extracted with EtOAc (3×10 mL). The combined organic layers were washed with brine (15 mL) and dried over anhydrous Na₂SO₄. The resulting mixture was concentrated under reduced pressure. The residue was purified by silica gel column and eluted with 4% MeOH in DCM to afford tert-butyl 8-methyl-7-[7-({6-[2-(morpholin-4-yl)acetamido]pyridin-3-yl}amino)-1,2,3,4-tetrahydro-2,6-naphthyridin-2-yl]-1H,2H,3H-pyrido[2,3-b][1,4]oxazine-1-carboxylate (120.00 mg; 27.5%) as a yellow solid.

N-{5-[(6-{8-methyl-1H,2H,3H-pyrido[2,3-b][1,4]oxazin-7-yl}-5,6,7,8-tetrahydro-2,6-naphthyridin-3-yl)amino]pyridin-2-yl}-2-(morpholin-4-yl)acetamide: To a stirred solution of tert-butyl 8-methyl-7-[7-({6-[2-(morpholin-4-yl)acetamido]pyridin-3-yl}amino)-1,2,3,4-tetrahydro-2,6-naphthyridin-2-yl]-1H,2H,3H-pyrido[2,3-b][1,4]oxazine-1-carboxylate (100.00 mg; 1.62 mmol) in DCM (4.00 mL) was added TFA (0.80 mL) in portions at 100° C. under N₂ atmosphere. The resulting mixture was stirred for 1 h at room temperature under N₂ atmosphere. The resulting mixture was concentrated under reduced pressure. The residue was purified by Prep-HPLC with the following condition (Column: X Bridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 25% B to 55% B in 9 min., 55% B; Wave Length: 254 nm; RT1 (min.): 7) to afford N-{5-[(6-{8-methyl-1H,2H,3H-pyrido[2,3-b][1,4]oxazin-7-yl}-5,6,7,8-tetrahydro-2,6-naphthyridin-3-yl)amino]pyridin-2-yl}-2-(morpholin-4-yl) acetamide (35.50 mg; 42.3%) as a white solid.

HPLC: 95.26% purity, RT=1.94 min. MS: m/z=517.30 [M+H]⁺. ¹H NMR (400 MHz, DMSO-d6) 9.74 (s, 1H), 8.97 (s, 1H), 8.56 (d, J=2.7 Hz, 1H), 8.07 (dd, J=8.9, 2.7 Hz, 1H), 8.02-7.94 (m, 2H), 7.26 (s, 1H), 6.57 (s, 1H), 5.50 (t, J=2.7 Hz, 1H), 4.18 (t, J=4.4 Hz, 2H), 3.99 (s, 2H), 3.63 (t, J=4.6 Hz, 4H), 3.33-3.26 (m, 2H), 3.16 (s, 2H), 3.09 (t, J=5.7 Hz, 2H), 2.82 (t, J=5.7 Hz, 2H), 2.54 (d, J=4.6 Hz, 4H), 2.05 (d, J=18.0 Hz, 3H).

### Example 33: Synthesis of Compound 33 2-(dimethylamino)-N-{2-methyl-4-[(6-{8-methyl-1H,2H,3H-pyrido[2,3-b][1,4]oxazin-7-yl}-5,6,7,8-tetrahydro-2,6-naphthyridin-3-yl) amino]phenyl}acetamide

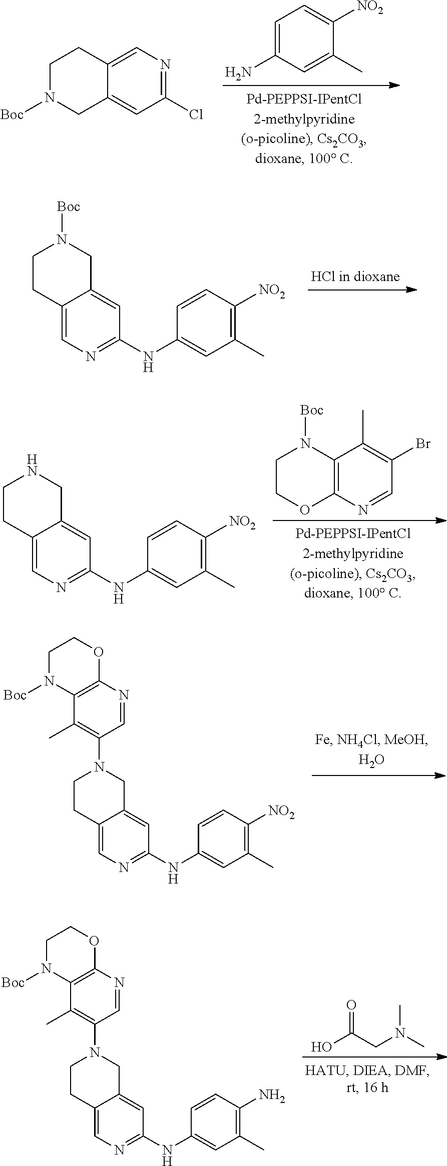

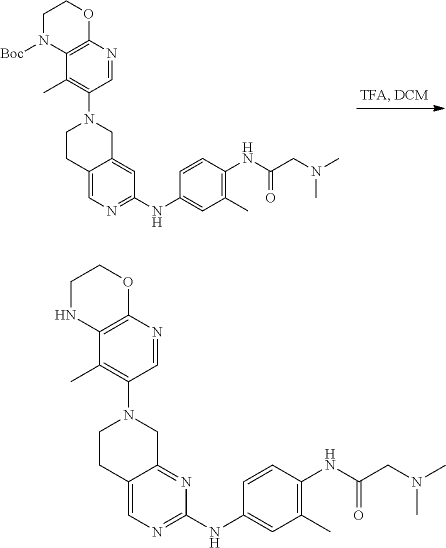 [SMILES: Cc1cc(Nc2cc3c(cn2)CCN(c2cnc4c(c2C)N(C(=O)OC(C)(C)C)CCO4)C3)ccc1NC(=O)CN(C)C ; Cc1cc(Nc2ncc3c(n2)CN(c2cnc4c(c2C)NCCO4)CC3)ccc1NC(=O)CN(C)C]

tert-butyl 7-[(3-methyl-4-nitrophenyl)amino]-1,2,3,4-tetrahydro-2,6-naphthyridine-2-carboxylate: To a stirred mixture of 3-methyl-4-nitroaniline (499.81 mg; 3.28 mmol; 1.50 eq.) and tert-butyl 7-chloro-1,2,3,4-tetrahydro-2,6-naphthyridine-2-carboxylate (600.00 mg; 2.19 mmol) in 1,4-dioxane (18.00 mL) and were added PEPPSI-IPr (142.44 mg; 0.22 mmol) and Cs₂CO₃ (1500.83 mg; 4.38 mmol) in portions at 25° C. The resulting mixture was stirred for 2 h at 100° C. under argon atmosphere. The resulting mixture was concentrated under reduced pressure. The residue was purified by silica gel column chromatography and eluted with CH₂Cl₂/MeOH (10:1) to afford tert-butyl 7-[(3-methyl-4-nitrophenyl)amino]-1,2,3,4-tetrahydro-2,6-naphthyridine-2-carboxylate (530.00 mg; 62.92%) as a light yellow solid.

N-(3-methyl-4-nitrophenyl)-5,6,7,8-tetrahydro-2,6-naphthyridin-3-amine: To a stirred mixture of tert-butyl 7-[(3-methyl-4-nitrophenyl)amino]-1,2,3,4-tetrahydro-2,6-naphthyridine-2-carboxylate (520.00 mg; 1.35 mmol) in DCM (10.00 mL) and was added TFA (2.00 mL) at room temperature. After stirring for 1 h at room temperature, the mixture was concentrated under reduced pressure. The resulting mixture was quenched with water. The mixture was acidified/basified to pH=8 with Na₂CO₃ (aq.). The resulting mixture was filtered and the filter cake was washed with water. The filter cake was concentrated under reduced pressure to afford N-(3-methyl-4-nitrophenyl)-5,6,7,8-tetrahydro-2,6-naphthyridin-3-amine (290.00 mg; 75.37%) as a yellow solid.

tert-butyl 8-methyl-7-{7-[(3-methyl-4-nitrophenyl)amino]-1,2,3,4-tetrahydro-2,6-naphthyridin-2-yl}-1H,2H,3H-pyrido[2,3-b][1,4]oxazine-1-carboxylate: To a solution of N-(3-methyl-4-nitrophenyl)-5,6,7,8-tetrahydro-2,6-naphthyridin-3-amine (280.00 mg; 0.98 mmol) and tert-butyl 7-bromo-8-methyl-1H,2H,3H-pyrido[2,3-b][1,4]oxazine-1-carboxylate (490.92 mg; 1.47 mmol) in 1,4-dioxane (15.00 mL) were added Cs₂CO₃ (1011.37 mg; 2.95 mmol) and Pd-PEPPSI-IPentCl 2-methylpyridine (o-picoline) (86.93 mg; 0.10 mmol). The resulting mixture was stirred for 24 h at 100° C. under nitrogen atmosphere. The mixture was concentrated under reduced pressure. The residue was purified by silica gel column chromatography and eluted with PE/EtOAc (1:2) to afford tert-butyl 8-methyl-7-{7-[(3-methyl-4-nitrophenyl)amino]-1,2,3,4-tetrahydro-2,6-naphthyridin-2-yl}-1H,2H,3H-pyrido[2,3-b][1,4]oxazine-1-carboxylate (430.00 mg; 75.69%) as a yellow solid.

tert-butyl 7-{7-[(4-amino-3-methylphenyl)amino]-1,2,3,4-tetrahydro-2,6-naphthyridin-2-yl}-8-methyl-1H,2H,3H-pyrido[2,3-b][1,4]oxazine-1-carboxylate: To a solution of tert-butyl 8-methyl-7-{7-[(3-methyl-4-nitrophenyl)amino]-1,2,3,4-tetrahydro-2,6-naphthyridin-2-yl}-1H,2H,3H-pyrido[2,3-b][1,4]oxazine-1-carboxylate (420.00 mg; 0.73 mmol) in MeOH (10.00 mL) was added Raney-Ni (186.79 mg; 0.22 mmol) at room temperature. The mixture stirring at room temperature for 2 h under H₂ atmosphere. The resulting mixture was filtered and the filter cake was washed with MeOH (50 mL), the filtrate was concentrated under reduced pressure to afford tert-butyl 7-{7-[(4-amino-3-methylphenyl)amino]-1,2,3,4-tetrahydro-2,6-naphthyridin-2-yl}-8-methyl-1H,2H,3H-pyrido[2,3-b][1,4]oxazine-1-carboxylate (330.00 mg; 86.84%) as a light yellow solid).

tert-butyl 7-[7-({4-[2-(dimethylamino)acetamido]-3-methylphenyl}amino)-1,2,3,4-tetrahydro-2,6-naphthyridin-2-yl]-8-methyl-1H,2H,3H-pyrido[2,3-b][1,4]oxazine-1-carboxylate: To a stirred mixture of tert-butyl 7-{7-[(4-amino-3-methylphenyl)amino]-1,2,3,4-tetrahydro-2,6-naphthyridin-2-yl}-8-methyl-1H,2H,3H-pyrido[2,3-b][1,4]oxazine-1-carboxylate (150.00 mg; 0.29 mmol) and 2-(dimethylamino) acetic acid (37.36 mg; 0.34 mmol) in DMF (15.00 mL) was added DIEA (0.16 mL; 0.86 mmol) at room temperature. Then the mixture cooled to 0° C. and HATU (229.61 mg; 0.57 mmol) was added slowly at 0° C. After stirring for 4 h at room temperature, the resulting mixture was quenched with water (20 mL) and extracted with EtOAc (20 mL*3). The combined organic layers were washed with brine (20 mL) and dried over anhydrous Na₂SO₄. After filtration, the filtrate was concentrated under reduced pressure. The residue was purified by silica gel column chromatography and eluted with CH₂Cl₂/MeOH (10:1) to afford tert-butyl 7-[7-({4-[2-(dimethylamino)acetamido]-3-methylphenyl}amino)-1,2,3,4-tetrahydro-2,6-naphthyridin-2-yl]-8-methyl-1H,2H,3H-pyrido[2,3-b][1,4]oxazine-1-carboxylate (120.00 mg; 70.34%) as a yellow solid.

2-(dimethylamino)-N-{2-methyl-4-[(6-{8-methyl-1H,2H,3H-pyrido[2,3-b][1,4]oxazin-7-yl}-5,6,7,8-tetrahydro-2,6-naphthyridin-3-yl)amino]phenyl}acetamide: To a stirred mixture of tert-butyl 7-[7-({4-[2-(dimethylamino)acetamido]-3-methylphenyl}amino)-1,2,3,4-tetrahydro-2,6-naphthyridin-2-yl]-8-methyl-1H,2H,3H-pyrido[2,3-b][1,4]oxazine-1-carboxylate (120.00 mg; 0.20 mmol) in DCM (5.00 mL) and was added TFA (1.00 mL) at room temperature. After stirring for 1 h at room temperature, the mixture was concentrated under reduced pressure. The crude product was purified by Prep-HPLC (Column: YMC-Actus Triart C18 ExRS, 30*150 mm, 5 μm; Mobile Phase A: water (10 mmol/L NH₄HCO₃+0.05% NH₄OH), Mobile Phase B: ACN; Flow rate: 60 mL/min.; Gradient: 25% B to 55% B in 8 min., 55% B; Wave Length: 254 nm; RT1 (min.): 7; Number Of Runs: 2) to afford 2-(dimethylamino)-N-{2-methyl-4-[(6-{8-methyl-1H,2H,3H-pyrido[2,3-b][1,4]oxazin-7-yl}-5,6,7,8-tetrahydro-2,6-naphthyridin-3-yl)amino]phenyl}acetamide (36.40 mg; 36.84%) as an off-white solid.

HPLC: 99.5% purity, RT=2.17 min. MS: m/z=488.30 [M+H]⁺ ¹H NMR (300 MHz, Methanol-d4): 7.86 (s, 1H), 7.63 (d, J=8.5 Hz, 1H), 7.36-7.28 (m, 2H), 7.27 (dd, J=8.5, 2.5 Hz, 1H), 6.95 (s, 1H), 4.43 (t, J=4.4 Hz, 2H), 4.20 (s, 2H), 4.01 (t, J=3.3 Hz, 2H), 3.98-3.90 (m, 4H), 3.51 (t, J=4.4 Hz, 2H), 3.25 (dd, J=11.4, 5.5 Hz, 6H), 2.98 (t, J=5.8 Hz, 2H), 2.35 (s, 3H), 2.22 (s, 3H).

### Example 34: Synthesis of Compound 34 N-{2-methyl-4-[(6-{8-methyl-1H,2H,3H-pyrido[2,3-b][1,4]oxazin-7-yl}-5,6,7,8-tetrahydro-2,6-naphthyridin-3-yl) amino]phenyl}-2-(morpholin-4-yl)acetamide

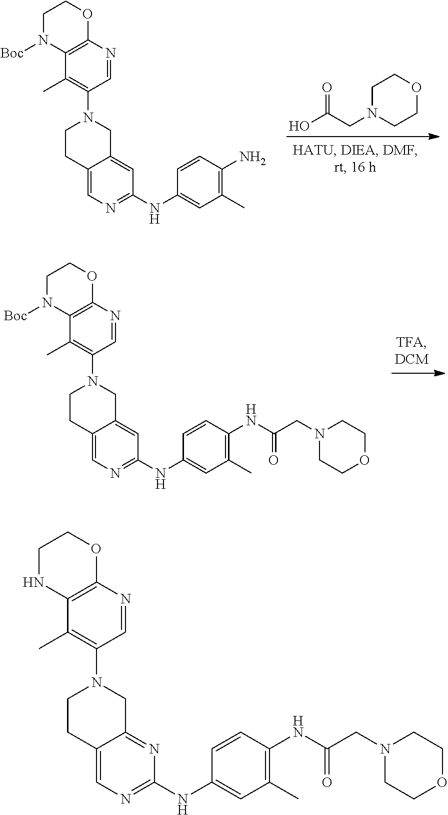 [SMILES: Cc1cc(Nc2cc3c(cn2)CCN(c2cnc4c(c2C)N(C(=O)OC(C)(C)C)CCO4)C3)ccc1N ; O=C(O)CN1CCOCC1 ; Cc1cc(Nc2cc3c(cn2)CCN(c2cnc4c(c2C)N(C(=O)OC(C)(C)C)CCO4)C3)ccc1NC(=O)CN1CCOCC1 ; Cc1cc(Nc2ncc3c(n2)CN(c2cnc4c(c2C)NCCO4)CC3)ccc1NC(=O)CN1CCOCC1]

tert-butyl 8-methyl-7-[7-({3-methyl-4-[2-(morpholin-4-yl)acetamido]phenyl}amino)-1,2,3,4-tetrahydro-2,6-naphthyridin-2-yl]-1H,2H,3H-pyrido[2,3-b][1,4]oxazine-1-carboxylate: To a stirred mixture of tert-butyl 7-{7-[(4-amino-3-methylphenyl)amino]-1,2,3,4-tetrahydro-2,6-naphthyridin-2-yl}-8-methyl-1H,2H,3H-pyrido[2,3-b][1,4]oxazine-1-carboxylate (120.00 mg; 0.23 mmol) and 2-(morpholin-4-yl)acetic acid (42.07 mg; 0.28 mmol) in DMF (12.00 mL) was added DIEA (0.13 mL; 0.69 mmol) at room temperature. Then the mixture cooled to 0° C. and HATU (183.68 mg; 0.46 mmol) was added slowly at 0° C. After stirring for 4 h at room temperature, the resulting mixture was quenched with water (20 mL) and extracted with EtOAc (20 mL*3). The combined organic layers were washed with brine (20 mL) and dried over anhydrous Na₂SO₄. After filtration, the filtrate was concentrated under reduced pressure. The residue was purified by silica gel column chromatography and eluted with CH₂Cl₂/MeOH (10:1) to afford tert-butyl 8-methyl-7-[7-({3-methyl-4-[2-(morpholin-4-yl)acetamido]phenyl}amino)-1,2,3,4-tetrahydro-2,6-naphthyridin-2-yl]-1H,2H,3H-pyrido[2,3-b][1,4]oxazine-1-carboxylate (140.00 mg; 94.85%) as a yellow solid.

N-{2-methyl-4-[(6-{8-methyl-1H,2H,3H-pyrido[2,3-b][1,4]oxazin-7-yl}-5,6,7,8-tetrahydro-2,6-naphthyridin-3-yl)amino]phenyl}-2-(morpholin-4-yl)acetamide: To a stirred mixture of tert-butyl 8-methyl-7-[7-({3-methyl-4-[2-(morpholin-4-yl)acetamido]phenyl}amino)-1,2,3,4-tetrahydro-2,6-naphthyridin-2-yl]-1H,2H,3H-pyrido[2,3-b][1,4]oxazine-1-carboxylate (130.00 mg; 0.20 mmol) in DCM (5.00 mL) and was added TFA (1.00 mL) at room temperature. After stirring for 1 h at room temperature, the mixture was concentrated under reduced pressure. The crude product was purified by Prep-HPLC (Column: XBridge Shield RP18 OBD Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 22% B to 52% B in 9 min., 52% B; Wave Length: 254 nm; RT1 (min.): 7) to afford N-{2-methyl-4-[(6-{8-methyl-1H,2H,3H-pyrido[2,3-b][1,4]oxazin-7-yl}-5,6,7,8-tetrahydro-2,6-naphthyridin-3-yl)amino]phenyl}-2-(morpholin-4-yl)acetamide (24.30 mg; 22.53%) as an off-white solid.

HPLC: 99.2% purity, RT=2.29 min. MS: m/z=530.25 [M+H]⁺ ¹H NMR (300 MHz, Methanol-d4): 7.86 (s, 1H), 7.63 (d, J=8.5 Hz, 1H), 7.36-7.28 (m, 2H), 7.27 (dd, J=8.5, 2.5 Hz, 1H), 6.95 (s, 1H), 4.43 (t, J=4.4 Hz, 2H), 4.20 (s, 2H), 4.01 (t, J=3.3 Hz, 2H), 3.98-3.90 (m, 4H), 3.51 (t, J=4.4 Hz, 2H), 3.25 (dd, J=11.4, 5.5 Hz, 6H), 2.98 (t, J=5.8 Hz, 2H), 2.35 (s, 3H), 2.22 (s, 3H).

### Example 35: Synthesis of Compound 35 2-methyl-4-[(7-{8-methyl-1H,2H,3H-pyrido[2,3-b][1,4]oxazin-7-yl}-5H,6H,7H,8H-pyrido[3,4-d]pyrimidin-2-yl)amino]-N-(1-methylazetidin-3-yl)benzamide

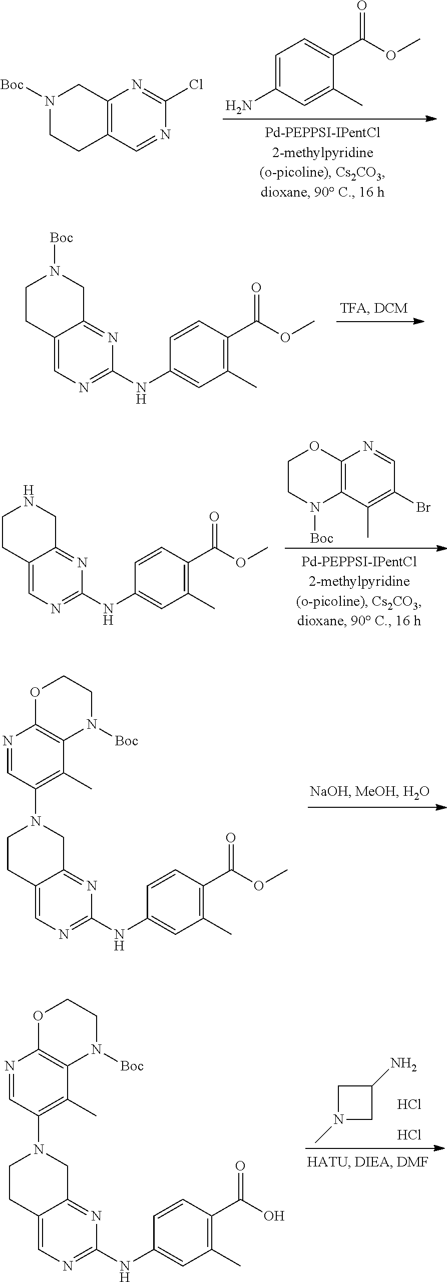

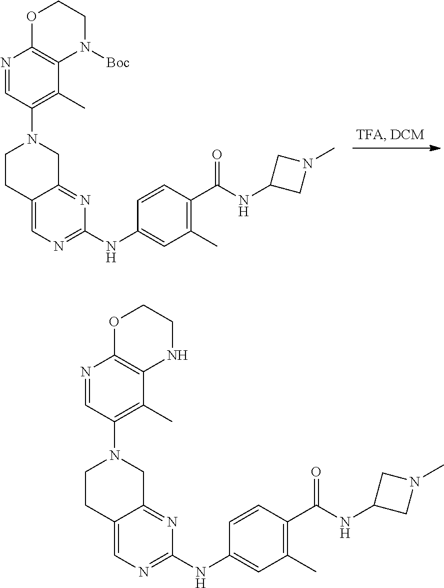 [SMILES: Cc1cc(Nc2ncc3c(n2)CN(c2cnc4c(c2C)N(C(=O)OC(C)(C)C)CCO4)CC3)ccc1C(=O)NC1CN(C)C1 ; Cc1cc(Nc2ncc3c(n2)CN(c2cnc4c(c2C)NCCO4)CC3)ccc1C(=O)NC1CN(C)C1]

tert-butyl 2{[-4-(methoxycarbonyl)-3-methylphenyl]amino}-5H,6H,7H,8H-pyrido[3,4-d]pyrimidine-7-carboxylate: To a stirred mixture of tert-butyl 2-chloro-5H,6H,7H,8H-pyrido[3,4-d]pyrimidine-7-carboxylate (1.00 g; 3.02 mmol) and methyl 4-amino-2-methylbenzoate (1.00 g; 6.04 mmol) in 1,4-dioxane (25.00 mL) were added Pd-PEPPSI-IPentCl 2-methylpyridine (o-picoline) (0.27 g; 0.30 mmol) and Cs₂CO₃ (2.07 g; 6.04 mmol) at room temperature. The mixture was stirred for 1 h at room temperature. The mixture was stirred for 2 h at 100° C. The resulting mixture was concentrated under reduced pressure and purified by silica gel column chromatography and eluted with PE/EtOAc (1:1) to afford tert-butyl 2-{[4-(methoxy carbonyl)-3-methylphenyl]amino}-5H,6H,7H,8H-pyrido[3,4-d]pyrimidine-7-carboxylate (1.20 g; 98.6%) as a yellow solid.

methyl 2-methyl-4-({5H,6H,7H,8H-pyrido[3,4-d]pyrimidin-2-yl}amino)benzoate: To a stirred mixture of tert-butyl 2-{[4-(methoxycarbonyl)-3-methylphenyl]amino}-5H,6H,7H,8H-pyrido[3,4-d]pyrimidine-7-carboxylate (1.26 g; 2.79 mmol; 1.00 eq.) and TFA (5.00 mL) in DCM (20.00 mL) at room temperature. The mixture was stirred for 1 h at room temperature. The mixture was basified to pH=9 with sat. NaHCO₃ (aq.). The resulting mixture was quenched by ice/water and extracted with EtOAc. The combined organic layers were washed with brine and dried over anhydrous Na₂SO₄. After filtration, the filtrate was concentrated under reduced pressure. This resulted in methyl 2-methyl-4-({5H,6H,7H,8H-pyrido[3,4-d]pyrimidin-2-yl}amino)benzoate (800.00 mg; 85.4%) as a yellow solid.

tert-butyl 7-(2-{[4-(methoxycarbonyl)-3-methylphenyl]amino}-5H,6H,7H,8H-pyrido[3,4-d]pyrimidin-7-yl)-8-methyl-1H,2H,3H-pyrido[2,3-b][1,4]oxazine-1-carboxylate: To a stirred mixture of methyl 2-methyl-4-({5H,6H,7H,8H-pyrido[3,4-d]pyrimidin-2-yl}amino)benzoate (1.00 g; 2.73 mmol) and tert-butyl 7-bromo-8-methyl-1H,2H,3H-pyrido[2,3-b][1,4]oxazine-1-carboxylate (1.80 g; 5.46 mmol) in 1,4-dioxane (20.00 mL) were added Pd-PEPPSI-IPentCl 2-methylpyridine (o-picoline) (0.24 g; 0.27 mmol) and Cs₂CO₃ (1.87 g; 5.46 mmol) at room temperature. The mixture was stirred for 2 h at 100° C. and concentrated under reduced pressure. The residue was purified by silica gel column chromatography and eluted with PE/EtOAc (1:1) to afford tert-butyl 7-(2-{[4-(methoxycarbonyl)-3-methylphenyl]amino}-5H,6H,7H,8H-pyrido[3,4-d]pyrimidin-7-yl)-8-methyl-1H,2H,3H-pyrido[2,3-b][1,4]oxazine-1-carboxylate (1.33 g; 86.8%) as a yellow solid.

4-[(7-{1-[(tert-butoxy)carbonyl]-8-methyl-1H,2H,3H-pyrido[2,3-b][1,4]oxazin-7-yl}-5H,6H,7H,8H-pyrido[3,4-d]pyrimidin-2-yl)amino]-2-methylbenzoic acid: To a stirred mixture of tert-butyl 7-(2-{[4-(methoxycarbonyl)-3-methylphenyl]amino}-5H,6H,7H,8H-pyrido[3,4-d]pyrimidin-7-yl)-8-methyl-1H,2H,3H-pyrido[2,3-b][1,4]oxazine-1-carboxylate (370.00 mg; 0.48 mmol; 1.00 eq.) and NaOH (60.96 mg; 1.45 mmol; 3.00 eq.) in MeOH (10.00 mL) and H₂O (3.00 mL) at room temperature. The mixture was stirred for 3 h at 50° C. The mixture was acidified to pH=7 with HCl (4 N) and extracted with EtOAc. The combined organic layers were washed with brine and dried over anhydrous Na₂SO₄. After filtration, the filtrate was concentrated under reduced pressure. This resulted in 4-[(7-{1-[(tert-butoxy)carbonyl]-8-methyl-1H,2H,3H-pyrido[2,3-b][1,4]oxazin-7-yl}-5H,6H,7H,8H-pyrido[3,4-d]pyrimidin-2-yl)amino]-2-methylbenzoic acid (980.00 mg, crude product) as a gray solid.

8-methyl-7-[2-({3-methyl-4-[(1-methylazetidin-3-yl)carbamoyl]phenyl}amino)-5H,6H,7H,8H-pyrido[3,4-d]pyrimidin-7-yl]-1H,2H,3H-pyrido[2,3-b][1,4]oxazine-1-carboxylate: To a stirred mixture of 4-[(7-{1-[(tert-butoxy)carbonyl]-8-methyl-1H,2H,3H-pyrido[2,3-b][1,4]oxazin-7-yl}-5H,6H,7H,8H-pyrido[3,4-d]pyrimidin-2-yl)amino]-2-methylbenzoic acid (150.00 mg; 0.23 mmol) and 1-methylazetidin-3-amine (39.49 mg; 0.46 mmol) in DMF (10.00 mL) were added HATU (275.27 mg; 0.69 mmol) and DIEA (62.36 mg; 0.46 mmol) at room temperature. The mixture was stirred for 1 h at room temperature. The resulting mixture was quenched by ice/water and extracted with EtOAc. The combined organic layers were washed with brine and dried over anhydrous Na₂SO₄. After filtration, the filtrate was concentrated under reduced pressure. The resulting mixture was concentrated under reduced pressure and purified by silica gel column chromatography and eluted with CH₂Cl₂/MeOH (10:1) to afford tert-butyl 8-methyl-7-[2-({3-methyl-4-[(1-methylazetidin-3-yl)carbamoyl]phenyl}amino)-5H,6H,7H,8H-pyrido[3,4-d]pyrimidin-7-yl]-1H,2H,3H-pyrido[2,3-b][1,4]oxazine-1-carboxylate (80.00 mg; 52.1%) as a yellow solid.

2-methyl-4-[(7-{8-methyl-1H,2H,3H-pyrido[2,3-b][1,4]oxazin-7-yl}-5H,6H,7H,8H-pyrido[3,4-d]pyrimidin-2-yl)amino]-N-(1-methylazetidin-3-yl)benzamide: A mixture of tert-butyl 8-methyl-7-[2-({3-methyl-4-[(1-methylazetidin-3-yl)carbamoyl]phenyl}amino)-5H,6H,7H,8H-pyrido[3,4-d]pyrimidin-7-yl]-1H,2H,3H-pyrido[2,3-b][1,4]oxazine-1-carboxylate (80.00 mg; 0.12 mmol) and TFA (1.00 mL) in DCM (5.00 mL) was stirred for 1 h at room temperature. The resulting mixture was concentrated under reduced pressure. The residue was purified by Prep-HPLC (Column: XBridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 22% B to 50% B in 9 min., 50% B; Wave Length: 254 nm; RTT (min.): 7) to afford 2-methyl-4-[(7-{8-methyl-1H,2H,3H-pyrido[2,3-b][1,4]oxazin-7-yl}-5H,6H,7H,8H-pyrido[3,4-d]pyrimidin-2-yl)amino]-N-(1-methylazetidin-3-yl)benzamide (22.70 mg; 23.4%) as a white solid.

HPLC: 98.2% purity, RT=2.45 min. MS: m/z=501.25 [M+H]⁺ ¹H NMR (300 MHz, DMSO-d6): 9.63 (s, 1H), 8.46 (d, J=7.2 Hz, 1H), 8.37 (s, 1H), 7.68 (dd, J=8.4, 2.2 Hz, 1H), 7.56 (d, J=2.2 Hz, 1H), 7.29 (t, J=4.2 Hz, 2H), 5.54 (d, J=2.7 Hz, 1H), 4.38 (h, J=7.1 Hz, 1H), 4.20 (t, J=4.2 Hz, 2H), 3.97 (s, 2H), 3.56 (t, J=7.3 Hz, 2H), 3.31 (d, J=3.6 Hz, 2H), 3.12 (d, J=5.8 Hz, 2H), 2.97-2.86 (m, 2H), 2.81 (d, J=5.6 Hz, 2H), 2.32 (s, 3H), 2.23 (s, 3H), 2.04 (s, 3H).

### Example 36: Synthesis of Compound 36 3-methyl-N5-(7-{8-methyl-1H,2H,3H-pyrido[2,3-b][1,4]oxazin-7-yl}-5H,6H,7H,8H-pyrido[3,4-d]pyrimidin-2-yl)-N2-[2-(morpholin-4-yl)ethyl]pyridine-2,5-diamine

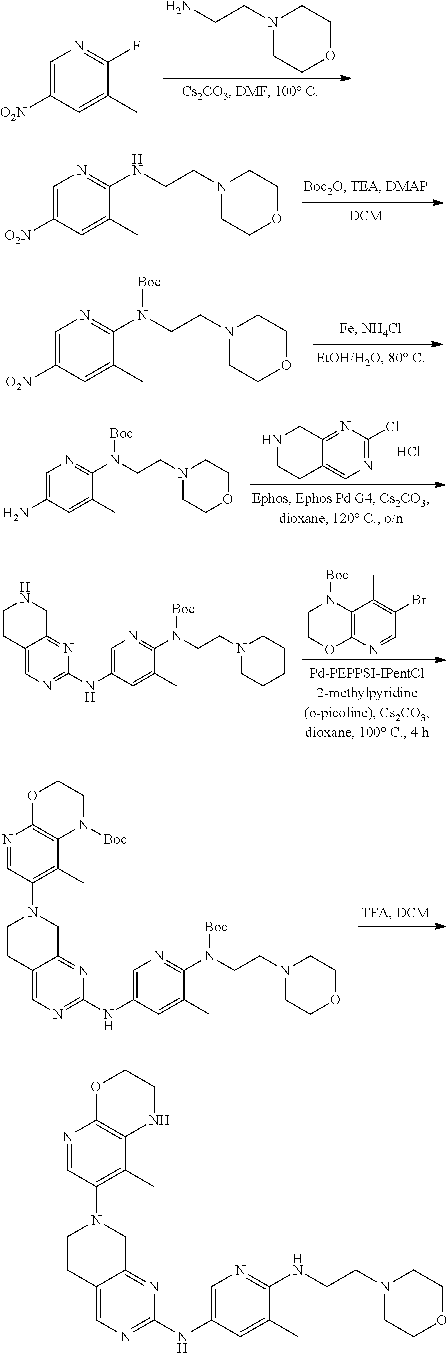

3-methyl-N-[2-(morpholin-4-yl)ethyl]-5-nitropyridin-2-amine: To a stirred solution of 2-fluoro-3-methyl-5-nitropyridine (2.50 g; 15.21 mmol), Cs₂CO₃ (10.01 g; 30.43 mmol) in DMF (25.00 mL) was added 2-(morpholin-4-yl)ethan-1-amine (2.50 g; 18.26 mmol) at room temperature. The resulting mixture was stirred for 2 h at 100° C. The reaction was quenched with water at room temperature. The resulting mixture was extracted with EtOAc (3×50 mL). The combined organic layers were washed with brine (1×30 mL) and dried over anhydrous Na₂SO₄. After filtration, the filtrate was concentrated under reduced pressure. This resulted in 3-methyl-N-[2-(morpholin-4-yl)ethyl]-5-nitropyridin-2-amine (3.00 g; crude) as a brown yellow solid.

tert-butyl N-(3-methyl-5-nitropyridin-2-yl)-N-[2-(morpholin-4-yl)ethyl]carbamate: A solution of 3-methyl-N-[2-(morpholin-4-yl)ethyl]-5-nitropyridin-2-amine (2.10 g; 7.89 mmol), Boc₂O (5.00 g; 22.68 mmol), DMAP (100.00 mg; 0.85 mmol) and TEA (3.43 mL; 23.47 mmol) in DCM (10.00 mL) was stirred for overnight at 30° C. under nitrogen atmosphere. The resulting mixture was concentrated under vacuum. The residue was purified by silica gel column chromatography and eluted with (PE:EtOAc=7:3) to afford tert-butyl N-(3-methyl-5-nitropyridin-2-yl)-N-[2-(morpholin-4-yl)ethyl]carbamate (1.10 g; 34.6%) as a yellow solid.

tert-butyl N-(5-amino-3-methylpyridin-2-yl)-N-[2-(morpholin-4-yl)ethyl]carbamate: A solution of tert-butyl N-(3-methyl-5-nitropyridin-2-yl)-N-[2-(morpholin-4-yl)ethyl]carbamate (1.10 g; 3.00 mmol), NH₄Cl (810.00 mg; 14.99 mmol) and Fe (810.00 mg; 14.21 mmol) in EtOH (12.00 mL) and water (6.00 mL) was stirred for 2 h at 80° C. under nitrogen atmosphere. The resulting mixture was filtered and the filter cake was washed with water. The filtrate was concentrated under reduced pressure. The residue was purified by silica gel column and eluted with DCM/MeOH=7:1 to afford tert-butyl N-(5-amino-3-methylpyridin-2-yl)-N-[2-(morpholin-4-yl)ethyl]carbamate (1.00 g, 99.0%) as a yellow solid.

tert-butyl N-[3-methyl-5-({5H,6H,7H,8H-pyrido[3,4-d]pyrimidin-2-yl}amino)pyridin-2-yl]-N-[2-(morpholin-4-yl)ethyl]carbamate: To a stirred solution of tert-butyl N-(5-amino-3-methylpyridin-2-yl)-N-[2-(morpholin-4-yl)ethyl]carbamate (300.00 mg; 0.89 mmol) and 2-chloro-5H,6H,7H,8H-pyrido[3,4-d]pyrimidine hydrochloride (320.00 mg; 1.43 mmol) in 1,4-dioxane (5.00 mL) was added EPhos Pd G₄ (80.00 mg; 0.08 mmol), Ephos (80.00 mg; 0.14 mmol; 0.16) and Cs₂CO₃ (900.00 mg; 2.62 mmol) at room temperature. The resulting mixture was stirred for 3 h at 120° C. under nitrogen atmosphere. The resulting mixture was concentrated under vacuum. The residue was purified by silica gel column chromatography and eluted with (PE:EtOAc=7:3) to afford tert-butyl N-[3-methyl-5-({5H,6H,7H,8H-pyrido[3,4-d]pyrimidin-2-yl}amino)pyridin-2-yl]-N-[2-(morpholin-4-yl)ethyl]carbamate (370.00 mg; 68.4%) as a yellow solid.

tert-butyl 7-{2-[(6-{[(tert-butoxy)carbonyl][2-(morpholin-4-yl)ethyl]amino}-5-methylpyridin-3-yl)amino]-5H,6H,7H,8H-pyrido[3,4-d]pyrimidin-7-yl}-8-methyl-1H,2H,3H-pyrido[2,3-b][1,4]oxazine-1-carboxylate: A solution of tert-butyl N-[3-methyl-5-({5H,6H,7H,8H-pyrido[3,4-d]pyrimidin-2-yl}amino)pyridin-2-yl]-N-[2-(morpholin-4-yl)ethyl]carbamate (350.00 mg; 0.58 mmol), tert-butyl 7-bromo-8-methyl-1H,2H,3H-pyrido[2,3-b][1,4]oxazine-1-carboxylate (220.00 mg; 0.63 mmol), Pd-PEPPSI-IPentCl 2-methylpyridine (o-picoline) (63.00 mg; 0.07 mmol) and Cs₂CO₃ (732.00 mg; 2.13 mmol) in 1,4-dioxane (5.00 mL) was stirred for 4 h at 100° C. under nitrogen atmosphere. The resulting mixture was concentrated under vacuum. The residue was purified by silica gel column chromatography and eluted with (PE:EtOAc=7:3) to afford tert-butyl 7-{2-[(6-{[(tert-butoxy)carbonyl][2-(morpholin-4-yl)ethyl]amino}-5-methylpyridin-3-yl)amino]-5H,6H,7H,8H-pyrido[3,4-d]pyrimidin-7-yl}-8-methyl-1H,2H,3H-pyrido[2,3-b][1,4]oxazine-1-carboxylate (285.00 mg; 49.3%) as a yellow solid.

3-methyl-N5-(7-{8-methyl-1H,2H,3H-pyrido[2,3-b][1,4]oxazin-7-yl}-5H,6H,7H,8H-pyrido[3,4-d]pyrimidin-2-yl)-N2-[2-(morpholin-4-yl)ethyl]pyridine-2,5-diamine: A solution of tert-butyl 7-{2-[(6-{[(tert-butoxy)carbonyl][2-(morpholin-4-yl)ethyl]amino}-5-methylpyridin-3-yl)amino]-5H,6H,7H,8H-pyrido[3,4-d]pyrimidin-7-yl}-8-methyl-1H,2H,3H-pyrido[2,3-b][1,4]oxazine-1-carboxylate (275.00 mg; 0.34 mmol) and TFA (2.00 mL) in DCM (6.00 mL) was stirred for 6 h at room temperature under nitrogen atmosphere. The crude product was purified by Prep-HPLC with the following conditions: Column: YMC-Actus Triart C18 ExRS, 30*150 mm, 5 μm; Mobile Phase A: water (10 mmol/L NH₄HCO₃+0.1% NH₄OH), Mobile Phase B: ACN; Flow rate: 60 m/min.; Gradient: 18% B to 48% B in 8 min., 48% B; Wave Length: 254 nm; RT1 (min.): 7). This resulted in 3-methyl-N5-(7-{8-methyl-1H,2H,3H-pyrido[2,3-b][1,4]oxazin-7-yl}-5H,6H,7H,8H-pyrido[3,4-d]pyrimidin-2-yl)-N2-[2-(morpholin-4-yl)ethyl]pyridine-2,5-diamine (42.50 mg, 21.9%) as an orange solid.

HPLC: 90.98% purity, RT=2.11 min. MS: m/z=518.40 [M+H]⁺ ¹H NMR (400 MHz, DMSO-d6) 8.95 (s, 1H), 8.22 (s, 1H), 8.16 (d, J=2.5 Hz, 1H), 7.47 (d, J=2.5 Hz, 1H), 7.26 (s, 1H), 5.53-5.42 (m, 2H), 4.18 (t, J=4.4 Hz, 2H), 3.89 (s, 2H), 3.58 (t, J=4.7 Hz, 5H), 3.42 (q, J=6.4 Hz, 2H), 3.09 (d, J=6.1 Hz, 2H), 2.74 (s, 2H), 2.41 (s, 5H), 2.03 (s, 6H).

### Example 37: Synthesis of Compound 37 2-[(6-{8-chloro-1H,2H,3H-pyrido[2,3-b][1,4]oxazin-7-yl}-5,6,7,8-tetrahydro-2,6-naphthyridin-3-yl)amino]-6-methyl-4H,5H,6H,7H,8H-pyrazolo[1,5-d][1,4]diazepin-7-one

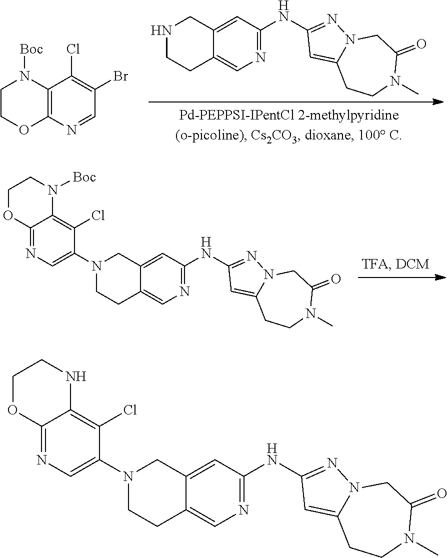 [SMILES: CC(C)(C)OC(=O)N1CCOc2ncc(Br)c(Cl)c21 ; CN1CCc2cc(Nc3cc4c(cn3)CCNC4)nn2CC1=O ; CN1CCc2cc(Nc3cc4c(cn3)CCN(c3cnc5c(c3Cl)N(C(=O)OC(C)(C)C)CCO5)C4)nn2CC1=O ; CN1CCc2cc(Nc3cc4c(cn3)CCN(c3cnc5c(c3Cl)NCCO5)C4)nn2CC1=O]

tert-butyl 8-chloro-7-[7-({6-methyl-7-oxo-4H,5H,6H,7H,8H-pyrazolo[1,5-d][1,4]diazepin-2-yl}amino)-1,2,3,4-tetrahydro-2,6-naphthyridin-2-yl]-1H,2H,3H-pyrido[2,3-b][1,4]oxazine-1-carboxylate: To a stirred solution of tert-butyl 7-bromo-8-chloro-1H,2H,3H-pyrido[2,3-b][1,4]oxazine-1-carboxylate (200.00 mg; 0.53 mmol) and 6-methyl-2-[(5,6,7,8-tetrahydro-2,6-naphthyridin-3-yl)amino]-4H,5H,6H,7H,8H-pyrazolo[1,5-d][1,4]diazepin-7-one (182.67 mg; 0.53 mmol) in 1,4-dioxane (5.00 mL) was added Cs₂CO₃ (541.52 mg; 1.58 mmol) and Pd-PEPPSI-IPentCl 2-methylpyridine (o-picoline) (46.54 mg; 0.05 mmol) in portions at room temperature under N₂ atmosphere. The resulting mixture was stirred for overnight at 100° C. under N₂ atmosphere. The resulting mixture was concentrated under reduced pressure. The residue was purified with silica gel column and eluted with 8% MeOH in DCM to afford crude product. The crude product was re-purified by C18 flash column and eluted with 45% ACN in water to afford tert-butyl 8-chloro-7-[7-({6-methyl-7-oxo-4H,5H,6H,7H,8H-pyrazolo[1,5-d][1,4]diazepin-2-yl}amino)-1,2,3,4-tetrahydro-2,6-naphthyridin-2-yl]-1H,2H,3H-pyrido[2,3-b][1,4]oxazine-1-carboxylate (160.00 mg; 36.17%) as a yellow solid.

2-[(6-{8-chloro-1H,2H,3H-pyrido[2,3-b][1,4]oxazin-7-yl}-5,6,7,8-tetrahydro-2,6-naphthyridin-3-yl)amino]-6-methyl-4H,5H,6H,7H,8H-pyrazolo[1,5-d][1,4]diazepin-7-one: To a stirred solution of tert-butyl 8-chloro-7-[7-({6-methyl-7-oxo-4H,5H,6H,7H,8H-pyrazolo[1,5-d][1,4]diazepin-2-yl}amino)-1,2,3,4-tetrahydro-2,6-naphthyridin-2-yl]-1H,2H,3H-pyrido[2,3-b][1,4]oxazine-1-carboxylate (150.00 mg; 0.26 mmol) in DCM (3.00 mL) was added TFA (1.00 mL) dropwise at room temperature under N₂ atmosphere. The resulting mixture was stirred for 2 h at 25° C. under N₂ atmosphere. The residue was purified by Prep-HPLC with the following condition (Column: XBridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 18% B to 48% B in 8 min., 48% B; Wave Length: 254 nm; RTT (min.): 7), to afford 2-[(6-{8-chloro-1H,2H,3H-pyrido[2,3-b][1,4]oxazin-7-yl}-5,6,7,8-tetrahydro-2,6-naphthyridin-3-yl)amino]-6-methyl-4H,5H,6H,7H,8H-pyrazolo[1,5-d][1,4]diazepin-7-one (33.80 mg; 26.73%) as an off-white solid.

HPLC: 98.23% purity, RT=2.76 min. MS: m/z=481.10 [M+H]⁺ ¹H NMR (300 MHz, DMSO-d6) δ 9.00 (s, 1H), 7.94 (s, 1H), 7.28 (s, 1H), 7.01 (s, 1H), 6.18 (s, 1H), 6.08 (s, 1H), 4.91 (s, 2H), 4.23 (s, 2H), 4.09 (s, 2H), 3.81 (s, 2H), 3.20 (s, 3H), 3.02 (s, 2H), 2.92 (m, 2H), 2.77 (s, 3H), 2.51 (s, 1H).

### Example 38: Synthesis of Compound 38 N,N-dimethyl-1-{2-methyl-4-[(7-{8-methyl-1H,2H,3H-pyrido[2,3-b][1,4]oxazin-7-yl}-5H,6H,7H,8H-pyrido[3,4-d]pyrimidin-2yl) amino]benzoyl}azetidin-3-amin

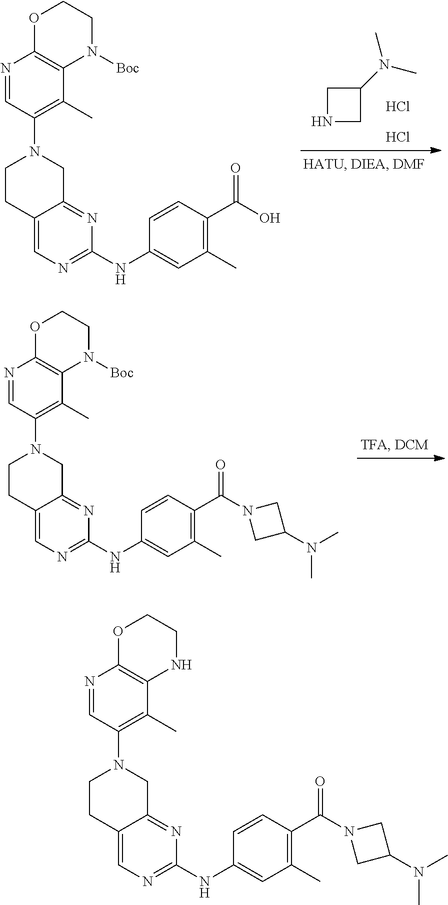

tert-butyl 7-[2-({4-[3-(dimethylamino) azetidine-1-carbonyl]-3-methyl phenyl}amino)-5H,6H,7H,8H-pyrido[3,4-d]pyrimidin-7-yl]-8-methyl-1H,2H,3H-pyrido[2,3-b][1,4]oxazine-1-carboxylate: To a stirred mixture of 4-[(7-{1-[(tert-butoxy) carbonyl]-8-methyl-1H,2H,3H-pyrido[2,3-b][1,4]oxazin-7-yl}-5H,6H,7H,8H-pyrido[3,4-d]pyrimidin-2-yl)amino]-2-methylbenzoic acid (150.00 mg; 0.23 mmol) and N,N-dimethylazetidin-3-amine (45.93 mg; 0.46 mmol) in DMF (10.00 mL) were added HATU (275.27 mg; 0.69 mmol) and DIEA (62.36 mg; 0.46 mmol) at room temperature. The mixture was stirred for 1 h at room temperature. The resulting mixture was quenched by ice/water and extracted with EtOAc. The combined organic layers were washed with brine and dried over anhydrous Na₂SO₄. After filtration, the filtrate was concentrated under reduced pressure. The resulting mixture was concentrated under reduced pressure and purified by silica gel column chromatography and eluted with CH₂Cl₂/MeOH (10:1) to afford tert-butyl 7-[2-({4-[3-(dimethylamino) azetidine-1-carbonyl]-3-methyl phenyl}amino)-5H,6H,7H,8H-pyrido[3,4-d]pyrimidin-7-yl]-8-methyl-1H,2H,3H-pyrido[2,3-b][1,4]oxazine-1-carboxylate (100.00 mg; 67.9%) as a blue solid.

N, N-dimethyl-1-{2-methyl-4-[(7-{8-methyl-1H,2H,3H-pyrido[2,3-b][,4]oxazin-7-yl}-5H,6H,7H,8H-pyrido[3,4-d]pyrimidin-2yl) amino]benzoyl}azetidin-3-amine: To a stirred mixture of tert-butyl 7-[2-({4-[3-(dimethylamino) azetidine-1-carbonyl]-3-methylphenyl}amino)-5H,6H,7H,8H-pyrido[3,4-d]pyrimidin-7-yl]-8-methyl-1H,2H,3H-pyrido[2,3-b][1,4]oxazine-1-carboxylate (160.00 mg; 0.23 mmol) and TFA (1.00 mL) in DCM (5.00 mL) at room temperature. The mixture was stirred for 1 h at room temperature. The resulting mixture was concentrated under reduced pressure. The residue was purified by Prep-HPLC (Column: XBridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 22% B to 50% B in 9 min., 50% B; Wave Length: 254 nm; RTT (min.): 7) to afford N,N-dimethyl-1-{2-methyl-4-[(7-{8-methyl-1H,2H,3H-pyrido[2,3-b][1,4]oxazin-7-yl}-5H,6H,7H,8H-pyrido[3,4-d]pyrimidin-2yl) amino]benzoyl}azetidin-3-amine (26.70 mg; 22.4%) as a green solid.

HPLC: 98.83% purity, RT=2.33 min. MS: m/z=515.25 [M+H]⁺ ¹H NMR (300 MHz, Methanol-d4): 8.31 (s, 1H), 7.72 (dd, J=8.3, 2.2 Hz, 1H), 7.58 (d, J=2.2 Hz, 1H), 7.30 (s, 1H), 7.24 (d, J=8.4 Hz, 1H), 4.37-4.28 (m, 2H), 4.28-4.16 (m, 1H), 4.14-4.03 (m, 1H), 4.03 (s, 2H), 3.98 (dd, J=10.6, 5.1 Hz, 1H), 3.86 (dd, J=9.6, 5.1 Hz, 1H), 3.44 (t, J=4.4 Hz, 2H), 3.21 (dt, J=13.0, 6.4 Hz, 3H), 2.89 (t, J=5.4 Hz, 2H), 2.38 (s, 3H), 2.19 (d, J=13.1 Hz, 9H).

### Example 39: Synthesis of 2-methyl-4-[(7-{8-methyl-1H,2H,3H-pyrido[2,3-b]oxazin-7-yl}-5H,6H,7H,8H-pyrido[3,4-d]pyrimidin-2-yl) amino]-N-[2-(morpholin-4-yl)ethyl] benzamide

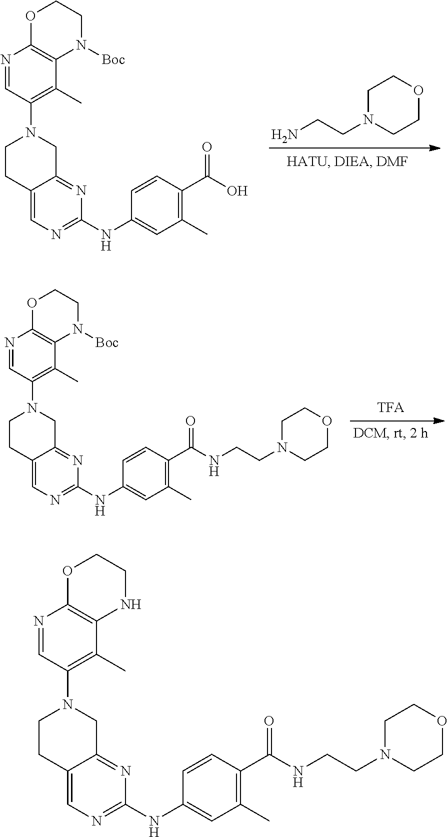 [SMILES: Cc1cc(Nc2ncc3c(n2)CN(c2cnc4c(c2C)N(C(=O)OC(C)(C)C)CCO4)CC3)ccc1C(=O)O ; NCCN1CCOCC1 ; Cc1cc(Nc2ncc3c(n2)CN(c2cnc4c(c2C)N(C(=O)OC(C)(C)C)CCO4)CC3)ccc1C(=O)NCCN1CCOCC1 ; Cc1cc(Nc2ncc3c(n2)CN(c2cnc4c(c2C)NCCO4)CC3)ccc1C(=O)NCCN1CCOCC1]

tert-butyl 8-methyl-7-{2-[(3-methyl-4-{[2-(morpholin-4-yl)ethyl]carbamoyl}phenyl) amino]-5H,6H,7H,8H-pyrido[3,4-d]pyrimidin-7-yl}-1H,2H,3H-pyrido[2,3-b][1,4]oxazine-1-carboxylate: To a stirred mixture of 4-[(7-{1-[(tert-butoxy) carbonyl]-8-methyl-1H,2H,3H-pyrido[2,3-b][1,4]oxazin-7-yl}-5H,6H,7H,8H-pyrido[3,4-d]pyrimidin-2-yl)amino]-2-methylbenzoic acid (150.00 mg; 0.23 mmol) and 2-(morpholin-4-yl)ethan-1-amine (59.69 mg; 0.46 mmol) in DMF (10.00 mL) were added HATU (275.27 mg; 0.69 mmol) and DIEA (62.36 mg; 0.46 mmol) at room temperature. The mixture was stirred for 1 h at room temperature. The resulting mixture was quenched by ice/water and extracted with EtOAc. The combined organic layers were washed with brine and dried over anhydrous Na₂SO₄. After filtration, the filtrate was concentrated under reduced pressure. The resulting mixture was concentrated under reduced pressure and purified by silica gel column chromatography and eluted with CH₂Cl₂/MeOH (10:1) to afford tert-butyl 8-methyl-7-{2-[(3-methyl-4-{[2-(morpholin-4-yl)ethyl] carbamoyl}phenyl) amino]-5H,6H,7H,8H-pyrido[3,4-d]pyrimidin-7-yl}-1H,2H,3H-pyrido[2,3-b][1,4]oxazine-1-carboxylate (150.00 mg; 98.4%) as a yellow solid.

2-methyl-4-[(7-{8-methyl-1H,2H,3H-pyrido[2,3-b][1,4]oxazin-7-yl}-5H,6H,7H,8H-pyrido[3,4-d]pyrimidin-2-yl) amino]-N-[2-(morpholin-4-yl) ethyl] benzamide: To a stirred mixture of tert-butyl 8-methyl-7-{2-[(3-methyl-4-{[2-(morpholin-4-yl)ethyl] carbamoyl}phenyl) amino]-5H,6H,7H,8H-pyrido[3,4-d]pyrimidin-7-yl}-1H,2H,3H-pyrido[2,3-b][1,4]oxazine-1-carboxylate (190.00 mg; 0.26 mmol) in DCM (5.00 mL) was added TFA (1.00 mL) at room temperature. The mixture was stirred for 1 h at room temperature. The resulting mixture was concentrated under reduced pressure. The residue was purified by Prep-HPLC (Column: XBridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 22% B to 50% B in 9 min., 50% B; Wave Length: 254 nm; RT1 (min.): 7) to afford 2-methyl-4-[(7-{8-methyl-1H,2H,3H-pyrido[2,3-b][1,4]oxazin-7-yl}-5H,6H,7H,8H-pyrido[3,4-d]pyrimidin-2-yl)amino]-N-[2-(morpholin-4-yl) ethyl]benzamide (25.50 mg; 17.7%) as a yellow solid.

HPLC: 98.02% purity, RT=2.46 min. MS: m/z=545.25 [M+H]⁺ ¹H NMR (300 MHz, DMSO-d6): 9.62 (s, 1H), 8.36 (s, 1H), 7.95 (t, J=5.7 Hz, 1H), 7.67 (dd, J=8.5, 2.2 Hz, 1H), 7.56 (d, J=2.2 Hz, 1H), 7.32-7.21 (m, 2H), 5.52 (d, J=3.2 Hz, 1H), 4.19 (t, J=4.3 Hz, 2H), 3.97 (s, 2H), 3.57 (t, J=4.6 Hz, 4H), 3.13 (t, J=5.6 Hz, 4H), 2.80 (t, J=5.5 Hz, 2H), 2.49-2.36 (m, 6H), 2.34 (s, 3H), 2.04 (s, 3H).

### Example 40: Synthesis of Compound 40 6-[(cyclopropanesulfonyl)methyl]-N-(7-{8-methyl-1H,2H,3H-pyrido[2,3-b][1,4]oxazin-7-yl}-5H,6H,7H,8H-pyrido[3,4-d]pyrimidin-2-yl) pyridin-3-amine

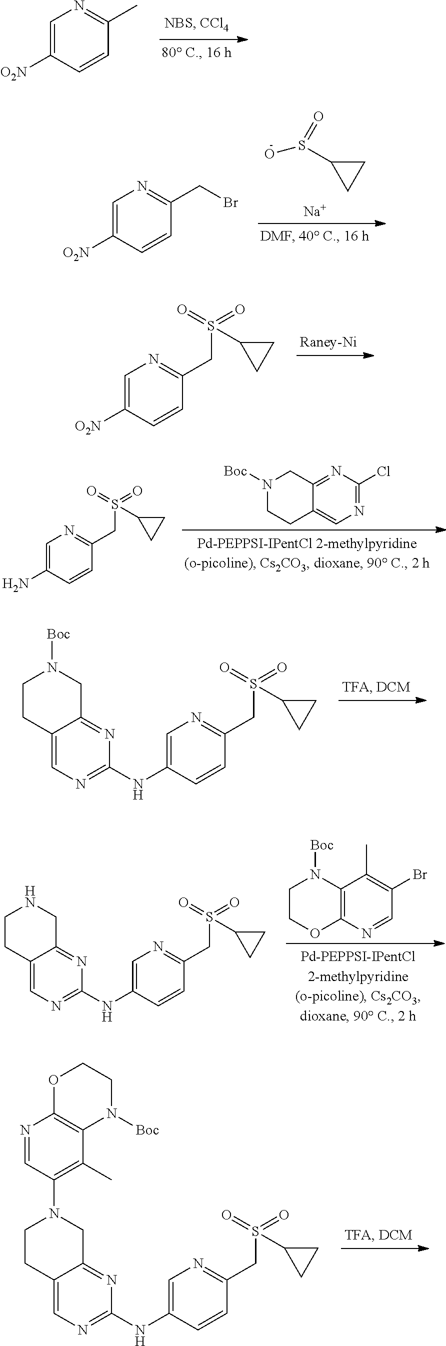

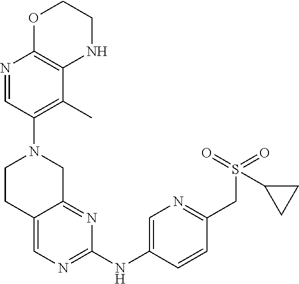 [SMILES: Cc1c(N2CCc3cnc(Nc4ccc(CS(=O)(=O)C5CC5)nc4)nc3C2)cnc2c1NCCO2]

2-(bromomethyl)-5-nitropyridine: To a stirred solution of 2-methyl-5-nitropyridine (10.00 g; 70.95 mmol) in CCl₄ (100.00 mL) was added NBS (38.66 g; 212.85 mmol) and AIBN (4.76 g; 28.38 mmol) in portions at room temperature. After stirring for 16 h at 80° C., the resulting mixture was concentrated under reduced pressure. The residue was purified by silica gel column chromatography and eluted with PE/EA (6:1) to afford 2-(bromomethyl)-5-nitropyridine (6.20 g; 31.65%) as a black solid.

2-[(cyclopropanesulfonyl)methyl]-5-nitropyridin: To a stirred solution of 2-(bromomethyl)-5-nitropyridine (1.00 g; 3.62 mmol) and sodium cyclopropanesulfinate (0.98 g; 7.24 mmol) in DMF (8.00 mL) was added sodium cyclopropanesulfinate (0.98 g; 7.24 mmol). The resulting mixture was stirred for 2 h at 65° C. under nitrogen atmosphere. The resulting mixture was concentrated under reduced pressure. The residue was purified by silica gel column chromatography and eluted with PE/EA (3:1) to afford 2-[(cyclopropanesulfonyl)methyl]-5-nitropyridine (806.00 mg; 90.80%) as a yellow solid.

6-[(cyclopropanesulfonyl)methyl]pyridin-3-amine: To a solution of 2-[(cyclopropanesulfonyl)methyl]-5-nitropyridine (1.00 g; 4.08 mmol) in MeOH (20.00 mL) was added Raney-Ni (423.14 mg; 4.69 mmol) at room temperature. After stirring for 1 h at room temperature under hydrogen atmosphere, the resulting mixture was filtered and the filter cake was washed with MeOH and then filtrate was concentrated under reduced pressure to afford 6-[(cyclopropanesulfonyl)methyl]pyridin-3-amine (743.00 mg; 84.82%) as a white solid.

tert-butyl 2-({6-[(cyclopropanesulfonyl) methyl]pyridin-3-yl}amino)-5H,6H,7H,8H-pyrido[3,4-d]pyrimidine-7-carboxylate: To a stirred solution of tert-butyl 2-chloro-5H,6H,7H,8H-pyrido[3,4-d]pyrimidine-7-carboxylate (200.00 mg; 0.64 mmol) and 6-[(cyclopropanesulfonyl) methyl]pyridin-3-amine (137.87 mg; 0.64 mmol) in 1,4-dioxane (10.00 mL) was added Pd-PEPPSI-IPentCl 2-methylpyridine (o-picoline) (56.79 mg; 0.06 mmol) and Cs₂CO₃ (440.46 mg; 1.28 mmol). The resulting mixture was stirred for 3 h at 100° C. under nitrogen atmosphere. The resulting mixture was concentrated under reduced pressure. The residue was purified by silica gel column chromatography and eluted with PE/EtOAc (1:1) to afford tert-butyl 2-({6-[(cyclopropanesulfonyl) methyl]pyridin-3-yl}amino)-5H,6H,7H,8H-pyrido[3,4-d]pyrimidine-7-carboxylate (250.00 mg; 72.94%) as a yellow solid.

6-[(cyclopropanesulfonyl)methyl]-N-{5H,6H,7H,8H-pyrido[3,4-d]pyrimidin-2-yl}pyridin-3-amine: To a stirred solution of tert-butyl 2-({6-[(cyclopropanesulfonyl) methyl]pyridin-3-yl}amino)-5H,6H,7H,8H-pyrido[3,4-d]pyrimidine-7-carboxylate (250.00 mg; 0.47 mmol) in DCM (10.00 mL) was added TFA (2.00 mL) in portions at room temperature. After stirring for 1 h at room temperature, the resulting mixture was concentrated under reduced pressure to afford 6-[(cyclopropanesulfonyl)methyl]-N-{5H,6H,7H,8H-pyrido[3,4-d]pyrimidin-2-yl}pyridin-3-amine (110.00 mg; 57.94%) as a yellow solid.

tert-butyl 7-[2-({6-[(cyclopropanesulfonyl)methyl]pyridin-3-yl}amino)-5H,6H,7H,8H-pyrido[3,4-d]pyrimidin-7-yl]-8-methyl-1H,2H,3H-pyrido[2,3-b][1,4]oxazine-1-carboxylate: To a stirred solution of 6-[(cyclopropanesulfonyl)methyl]-N-{5H,6H,7H,8H-pyrido[3,4-d]pyrimidin-2-yl}pyridin-3-amine (90.00 mg; 0.22 mmol) and tert-butyl 7-bromo-8-methyl-1H,2H,3H-pyrido[2,3-b][1,4]oxazine-1-carboxylate (76.87 mg; 0.22 mmol) in 1,4-dioxane (10.00 mL) was added Pd-PEPPSI-IPentCl 2-methylpyridine (o-picoline) (19.64 mg; 0.02 mmol) and Cs₂CO₃ (152.32 mg; 0.44 mmol). The resulting mixture was stirred for 3 h at 100° C. under nitrogen atmosphere. The resulting mixture was concentrated under reduced pressure. The residue was purified by silica gel column chromatography and eluted with CH₂Cl₂/MeOH (10:1) to afford tert-butyl 7-[2-({6-[(cyclopropanesulfonyl)methyl]pyridin-3-yl}amino)-5H,6H,7H,8H-pyrido[3,4-d]pyrimidin-7-yl]-8-methyl-1H,2H,3H-pyrido[2,3-b][1,4]oxazine-1-carboxylate (80.00 mg; 47.32%) as a yellow solid.

6-[(cyclopropanesulfonyl)methyl]-N-(7-{8-methyl-1H,2H,3H-pyrido [2,3-b][1,4]oxazin-7-yl}-5H,6H,7H,8H-pyrido[3,4-d]pyrimidin-2-yl) pyridin-3-amine: To a stirred solution of tert-butyl 7-[2-({6-[(cyclopropanesulfonyl) methyl]pyridin-3-yl}amino)-5H,6H,7H,8H-pyrido[3,4-d]pyrimidin-7-yl]-8-methyl-1H,2H,3H-pyrido[2,3-b][1,4]oxazine-1-carboxylate (80.00 mg; 0.11 mmol) in DCM (5.00 mL) was added TFA (1.00 mL) in portions at room temperature. After stirring for 1 h at room temperature, the resulting mixture was concentrated under reduced pressure. The crude product was purified by Prep-HPLC with the following conditions: Column: YMC-Actus Triart C18 ExRS, 30*150 mm, 5 μm; Mobile Phase A: water (10 mmol/L NH₄HCO₃+0.05% NH₄OH), Mobile Phase B: ACN; Flow rate: 60 mL/min.; Gradient: 30% B to 60% B in 8 min., 60% B; Wave Length: 254 nm; RT1 (min.): 7, to afford 6-[(cyclopropanesulfonyl)methyl]-N-(7-{8-methyl-1H,2H,3H-pyrido[2,3-b][1,4]oxazin-7-yl}-5H,6H,7H,8H-pyrido[3,4-d]pyrimidin-2-yl) pyridin-3-amine (16.60 mg; 31.82%) as a white solid.

HPLC: 99.41% purity, RT=2.79 min. MS: m/z=494.20 [M+H]⁺ ¹H NMR (300 MHz, Methanol-d4): 8.91 (d, J=2.7 Hz, 1H), 8.42-8.33 (m, 1H), 8.35 (s, 1H), 7.52 (d, J=8.6 Hz, 1H), 7.30 (s, 1H), 4.55 (s, 2H), 4.33 (t, J=4.5 Hz, 2H), 4.06 (s, 2H), 3.44 (t, J=4.4 Hz, 2H), 3.24 (t, J=5.6 Hz, 2H), 2.92 (d, J=5.8 Hz, 2H), 2.63-2.48 (m, 1H), 2.17 (s, 3H), 1.31 (s, 2H), 1.03 (dd, J=6.5, 3.4 Hz, 4H).

### Example 41: Synthesis of Compound 41 7-{8-methyl-1H,2H,3H-pyrido[2,3-b][1,4]oxazin-7-yl}-N-(4-{[(3-methyloxetan-3-yl) methanesulfonyl] methyl}phenyl)-5H,6H,7H,8H-pyrido[3,4-d]pyrimidin-2-amine

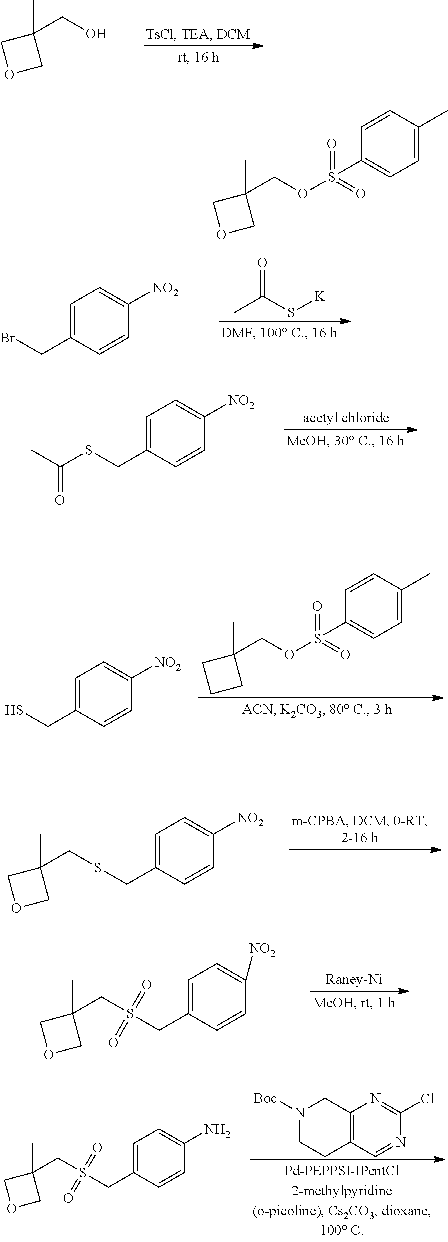

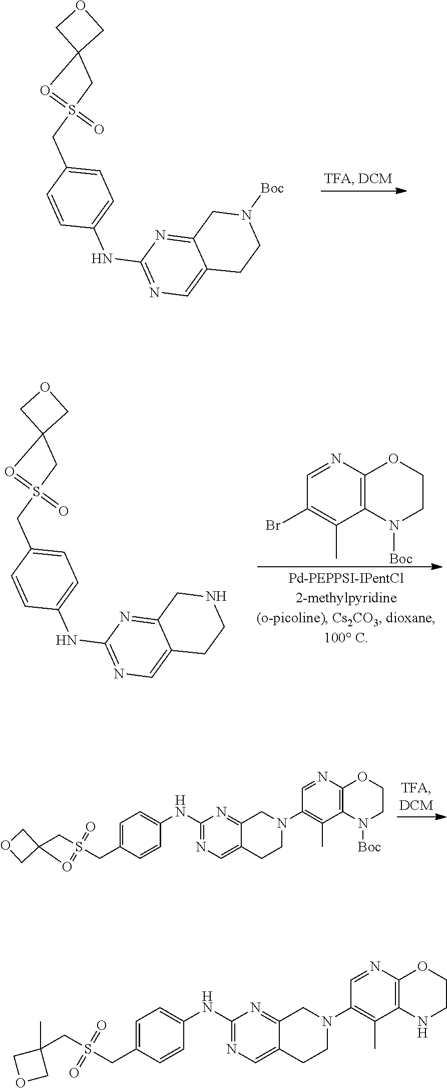

(3-methyloxetan-3-yl)methyl 4-methylbenzene-1-sulfonate: To a stirred solution of (3-methyloxetan-3-yl)methanol (5.00 g; 46.51 mmol) in DCM (60.00 mL) was added Et₃N (7.84 g; 69.76 mmol) and 4-methylbenzene-1-sulfonyl chloride (10.27 g; 51.16 mmol) in portions at room temperature. After stirring for 3 h at room temperature, the resulting mixture was concentrated under reduced pressure. The residue was purified by silica gel column chromatography and eluted with PE/EtOAc (1:1) to afford (3-methyloxetan-3-yl)methyl 4-methylbenzene-1-sulfonate (2.60 g; 21.49%) as a white solid.

1-{[(4-nitrophenyl)methyl]sulfanyl}ethan-1-one: To a stirred solution of 1-(bromomethyl)-4-nitrobenzene (4.00 g; 17.59 mmol) in DMF (40.00 mL) was added 1-(potassiosulfanyl)ethan-1-one (2.54 g; 21.11 mmol) in portions at room temperature. After stirring for 16 h at 100° C., the resulting mixture was extracted with EtOAc. The combined organic layers were washed with NaCl (aq.) and dried over anhydrous Na₂SO₄. After filtration, the filtrate was concentrated under reduced pressure to afford 1-{[(4-nitrophenyl)methyl]sulfanyl}ethan-1-one (3.20 g, 84.01%, Crude Product) as a black solid.

(4-nitrophenyl)methanethiol: To a stirred solution of 1-{[(4-nitrophenyl)methyl]sulfanyl}ethan-1-one (3.10 g; 14.32 mmol) in MeOH (10.00 mL) was added acetyl chloride (0.20 mL) in portions at room temperature. After stirring for 16 h at 30° C., the resulting mixture was concentrated under reduced pressure. The residue was purified by silica gel column chromatography and eluted with PE/EA (8:1) to afford (4-nitrophenyl)methanethiol (2.34 g; 93.08%) as a white solid.

3-methyl-3-({[(4-nitrophenyl)methyl]sulfanyl}methyl)oxetane: To a stirred solution of (4-nitrophenyl)methanethiol (1.50 g; 8.54 mmol) and (3-methyloxetan-3-yl)methyl 4-methylbenzene-1-sulfonate (2.67 g; 10.25 mmol) in ACN (30.00 mL) was added K₂CO₃ (2.48 g; 17.08 mmol) in portions at room temperature. After stirring for 3 h at 80° C., the resulting mixture was concentrated under reduced pressure. The residue was purified by silica gel column chromatography and eluted with PE/EA (6:1) to afford 3-methyl-3-({[(4-nitrophenyl)methyl]sulfanyl}methyl)oxetane (1.30 g; 55.57%) as a yellow solid.

3-methyl-3-{[(4-nitrophenyl)methanesulfonyl]methyl}oxetane: To a stirred solution of 3-methyl-3-({[(4-nitrophenyl)methyl]sulfanyl}methyl)oxetane (800.00 mg; 2.92 mmol) in DCM (10.00 mL) was added m-CPBA (2122.37 mg; 11.68 mmol). The resulting mixture was stirred for 2 h at 25° C. under nitrogen atmosphere. The resulting mixture was concentrated under reduced pressure. The residue was purified by silica gel column chromatography and eluted with PE/EA (1:1) to afford 3-methyl-3-{[(4-nitrophenyl)methanesulfonyl] methyl}oxetane (644.00 mg; 76.48%) as a white solid.

4-{[(3-methyloxetan-3-yl)methanesulfonyl]methyl}aniline: To a solution of 3-methyl-3-{[(4-nitrophenyl)methanesulfonyl]methyl}oxetane (900.00 mg; 3.12 mmol) in MeOH (20.00 mL) was added Raney-Ni (323.79 mg; 3.59 mmol) at room temperature. After stirring for 1 h at room temperature under hydrogen atmosphere, the resulting mixture was filtered. The filter cake was washed with MeOH and then filtrate was concentrated under reduced pressure to afford 4-{[(3-methyloxetan-3-yl)methanesulfonyl]methyl}aniline (660.00 mg; 56.15%, crude Product) as a yellow solid.

tert-butyl 2-[(4-{[(3-methyloxetan-3-yl)methanesulfonyl]methyl}phenyl)amino]-5H,6H,7H,8H-pyrido[3,4-d]pyrimidine-7-carboxylate: To a stirred solution of tert-butyl 2-chloro-5H,6H,7H,8H-pyrido[3,4-d]pyrimidine-7-carboxylate (220.00 mg; 0.71 mmol) and 4-{[(3-methyloxetan-3-yl)methanesulfonyl]methyl}aniline (265.97 mg; 0.71 mmol) in 1,4-dioxane (10.00 mL) was added Pd-PEPPSI-IPentCl 2-methylpyridine (o-picoline) (62.47 mg; 0.07 mmol) and Cs₂CO₃ (484.51 mg; 1.41 mmol). The resulting mixture was stirred for 3 h at 100° C. under nitrogen atmosphere. The resulting mixture was concentrated under reduced pressure. The residue was purified by silica gel column chromatography and eluted with PE/EtOAc (1:1) to afford tert-butyl 2-[(4-{[(3-methyloxetan-3-yl)methanesulfonyl]methyl}phenyl)amino]-5H,6H,7H,8H-pyrido[3,4-d]pyrimidine-7-carboxylate (300.00 mg; 86.26%) as a yellow solid.

N-(4-{[(3-methyloxetan-3-yl)methanesulfonyl]methyl}phenyl)-5H,6H,7H,8H-pyrido[3,4-d]pyrimidin-2-amine: To a stirred solution of tert-butyl 2-[(4-{[(3-methyloxetan-3-yl)methanesulfonyl]methyl}phenyl)amino]-5H,6H,7H,8H-pyrido[3,4-d]pyrimidine-7-carboxylate (300.00 mg; 0.61 mmol) in DCM (10.00 mL) was added TFA (2.00 mL) in portions at room temperature. After stirring for 1 h at room temperature, the resulting mixture was concentrated under reduced pressure to afford N-(4-{[(3-methyloxetan-3-yl)methanesulfonyl]methyl}phenyl)-5H,6H,7H,8H-pyrido[3,4-d]pyrimidin-2-amine (198.00 mg; crude product) as a yellow solid.

tert-butyl 8-methyl-7-{2-[(4-{[(3-methyloxetan-3-yl)methanesulfonyl]methyl}phenyl)amino]-5H,6H,7H,8H-pyrido[3,4-d]pyrimidin-7-yl}-1H,2H,3H-pyrido[2,3-b][1,4]oxazine-1-carboxylate: To a stirred solution of N-(4-{[(3-methyloxetan-3-yl)methanesulfonyl]methyl}phenyl)-5H,6H,7H,8H-pyrido[3,4-d]pyrimidin-2-amine (188.00 mg; 0.39 mmol) and tert-butyl 7-bromo-8-methyl-1H,2H,3H-pyrido[2,3-b][1,4]oxazine-1-carboxylate (135.00 mg; 0.39 mmol) in 1,4-dioxane (10.00 mL) was added Pd-PEPPSI-IPentCl 2-methylpyridine (o-picoline) (34.49 mg; 0.04 mmol) and Cs₂CO₃ (267.50 mg; 0.78 mmol). The resulting mixture was stirred for 3 h at 100° C. under nitrogen atmosphere. The resulting mixture was concentrated under reduced pressure. The residue was purified by silica gel column chromatography and eluted with CH₂Cl₂/MeOH (5:1) to afford tert-butyl 8-methyl-7-{2-[(4-{[(3-methyloxetan-3-yl)methanesulfonyl]methyl}phenyl)amino]-5H,6H,7H,8H-pyrido[3,4-d]pyrimidin-7-yl}-1H,2H,3H-pyrido[2,3-b][1,4]oxazine-1-carboxylate (170.00 mg, 59.06%) as a yellow solid.

7-{8-methyl-1H,2H,3H-pyrido[2,3-b][1,4]oxazin-7-yl}-N-(4-{[(3-methyloxetan-3-yl)methanesulfonyl]methyl}phenyl)-5H,6H,7H,8H-pyrido[3,4-d]pyrimidin-2-amine: To a stirred solution of tert-butyl 8-methyl-7-{2-[(4-{[(3-methyloxetan-3-yl)methanesulfonyl]methyl}phenyl)amino]-5H,6H,7H,8H-pyrido[3,4-d]pyrimidin-7-yl}-1H,2H,3H-pyrido[2,3-b][1,4]oxazine-1-carboxylate (170.00 mg; 0.23 mmol) in DCM (5.00 mL) was added TFA (1.00 mL) in portions at room temperature. After stirring for 1 h at room temperature, the resulting mixture was concentrated under reduced pressure. The crude product was purified by Prep-HPLC with the following conditions: Column: Xselect CSH C18 OBD Column 30*150 mm, 5 μm, n; Mobile Phase A: water (0.1% FA), Mobile Phase B: ACN; Flow rate: 60 mL/min.; Gradient: 7% B to 37% B in 8 min., 37% B; Wave Length: 254/220 nm; RT1 (min.): 6.8; Number Of Runs: 4, to afford 7-{8-methyl-1H,2H,3H-pyrido[2,3-b][1,4]oxazin-7-yl}-N-(4-{[(3-methyloxetan-3-yl)methanesulfonyl]methyl}phenyl)-5H,6H,7H,8H-pyrido[3,4-d]pyrimidin-2-amine; formic acid (9.90 mg 7.31%) as a white solid.

HPLC: 99.06% purity, RT=3.230 min. MS: m/z=537.20 [M+H]⁺. ¹H NMR (300 MHz, DMSO-d6): 9.65 (s, 1H), 8.36 (s, 1H), 7.79 (d, J=8.3 Hz, 2H), 7.34-7.26 (m, 3H), 5.53 (s, 1H), 4.54 (d, J=6.0 Hz, 2H), 4.39 (s, 2H), 4.20 (d, J=5.6 Hz, 4H), 4.19 (s, 2H), 3.98 (s, 2H), 3.55 (s, 2H), 3.13 (t, J=5.7 Hz, 2H), 2.81 (t, J=5.4 Hz, 2H), 2.05 (s, 3H), 1.51 (s, 3H).

### Example 42: Synthesis of Compound 42 (3R)—N,N-dimethyl-1-{4-[(7-{8-methyl-1H,2H,3H-pyrido[2,3-b][1,4]oxazin-7-yl} 5H,6H,7H,8H-pyrido[3,4-d]pyrimidin-2-yl)amino]benzoyl}pyrrolidin-3-amine

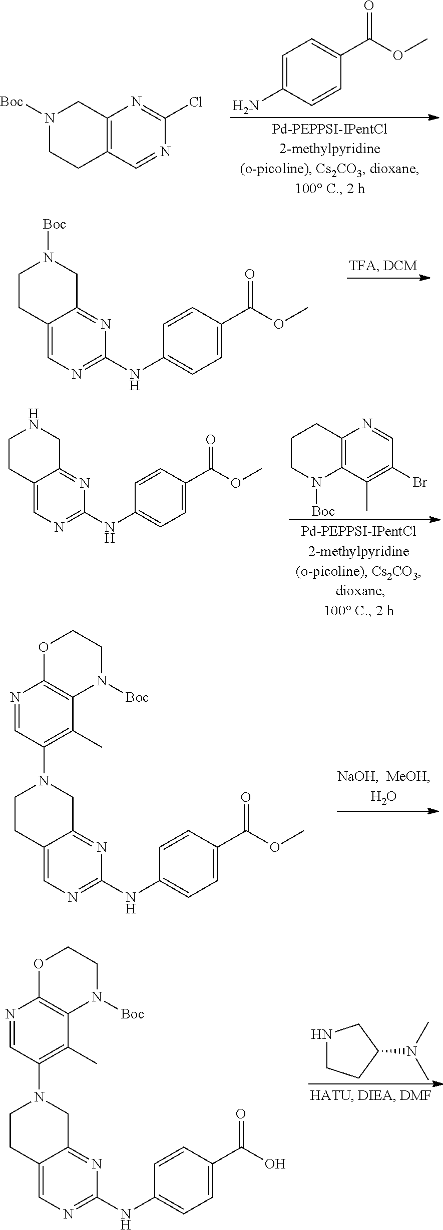

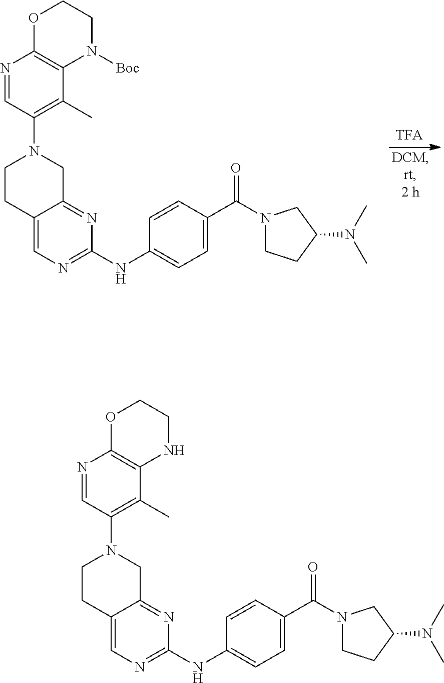 [SMILES: Cc1c(N2CCc3cnc(Nc4ccc(C(=O)N5CC[C@@H](N(C)C)C5)cc4)nc3C2)cnc2c1N(C(=O)OC(C)(C)C)CCO2 ; Cc1c(N2CCc3cnc(Nc4ccc(C(=O)N5CC[C@@H](N(C)C)C5)cc4)nc3C2)cnc2c1NCCO2]

tert-butyl 2-{[4(methoxycarbonyl)phenyl]amino}-5H,6H,7H,8H-pyrido[3,4-d]pyrimidine-7-carboxylate: To a stirred mixture of tert-butyl 2-chloro-5H,6H,7H,8H-pyrido[3,4-d]pyrimidine-7-carboxylate (800.00 mg; 2.41 mmol) and methyl 4-aminobenzoate (729.90 mg; 4.83 mmol) in 1,4-dioxane (20.00 mL) were added Pd-PEPPSI-IPentCl 2-methylpyridine (o-picoline) (213.51 mg; 0.24 mmol) and Cs₂CO₃ (1656.05 mg; 4.83 mmol) at room temperature. The mixture was stirred for 1 h at room temperature. The mixture was stirred for 2 h at 100° C. The resulting mixture was concentrated under reduced pressure and purified by silica gel column chromatography and eluted with PE/EtOAc (1:1) to afford tert-butyl 2-{[4(methoxycarbonyl)phenyl]amino}-5H,6H,7H,8H-pyrido[3,4-d]pyrimidine-7-carboxylate (800.00 mg 83.8%) as a yellow solid.

methyl 4-({5H,6H,7H,8H-pyrido[3,4-d]pyrimidin-2-yl}amino)benzoate: To a stirred mixture of tert-butyl 2-{[4-(methoxycarbonyl)phenyl]amino}-5H,6H,7H,8H-pyrido[3,4-d]pyrimidine-7-carboxylate (800.00 mg; 1.83 mmol) and TFA (4.00 mL) in DCM (20.00 mL) at room temperature. The mixture was stirred for 1 h at room temperature. The mixture was basified to pH=9 with sat. NaHCO₃ (aq.). The resulting mixture was quenched by ice/water and extracted with EtOAc. The combined organic layers were washed with brine and dried over anhydrous Na₂SO₄. After filtration, the filtrate was concentrated under reduced pressure. This resulted in methyl 4-({5H,6H,7H,8H-pyrido[3,4-d]pyrimidin-2-yl}amino)benzoate (350.00 mg, Crude Product) as a yellow solid.

tert-butyl 7-(2-{[4-(methoxycarbonyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate: To a stirred mixture of methyl 4-({5H,6H,7H,8H-pyrido[3,4-d]pyrimidin-2-yl}amino)benzoate (340.00 mg; 0.97 mmol) and tert-butyl 7-bromo-8-methyl-1H,2H,3H-pyrido[2,3-b][1,4]oxazine-1-carboxylate (640.89 mg; 1.95 mmol) in 1,4-dioxane (10.00 mL) were added Pd-PEPPSI-IPentCl 2-methylpyridine (o-picoline) (86.09 mg; 0.10 mmol) and Cs₂CO₃ (667.71 mg; 1.95 mmol) at room temperature. The mixture was stirred for 2 h at 100° C. The resulting mixture was concentrated under reduced pressure and purified by silica gel column chromatography and eluted with PE/EtOAc (1:1) to afford tert-butyl 7-(2-{[4-(methoxycarbonyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (380.00 mg, 67.1%) as a yellow solid.

4-[(7-{1-[(tert-butoxy)carbonyl]-8-methyl-1H,2H,3H-pyrido[2,3-b][1,4]oxazin-7-yl}-5H,6H,7H,8H-pyrido[3,4-d]pyrimidin-2-yl)amino]benzoic acid: A mixture of tert-butyl 7-(2-{[4-(methoxycarbonyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (370.00 mg; 0.50 mmol) and NaOH (62.56 mg; 1.49 mmol) in MeOH (10.00 mL) and H₂O (3.00 mL) was stirred for 3 h at 50° C. The mixture was acidified to pH=7 with HCl (4M.) and extracted with EtOAc. The combined organic layers were washed with brine and dried over anhydrous Na₂SO₄. After filtration, the filtrate was concentrated under reduced pressure. This resulted in 4-[(7-{1-[(tert-butoxy)carbonyl]-8-methyl-1H,2H,3H-pyrido[2,3-b][1,4]oxazin-7-yl}-5H,6H,7H,8H-pyrido[3,4-d]pyrimidin-2-yl)amino] benzoic acid (320.00 mg, crude product) as a yellow solid.

tert-butyl 7-[2-({4-[(3R)-3-(dimethylamino)pyrrolidine-1-carbonyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate: To a stirred mixture of 4-[(7-{1-[(tert-butoxy)carbonyl]-8-methyl-1H,2H,3H-pyrido[2,3-b][1,4]oxazin-7-yl}-5H,6H,7H,8H-pyrido[3,4-d]pyrimidin-2-yl)amino]benzoic acid (150.00 mg; 0.24 mmol) and (R)—N,N-dimethylpyrrolidin-3-amine (54.72 mg, 0.48 mmol) in DMF (10.00 mL) were added HATU (282.72 mg; 0.71 mmol) and DIEA (64.04 mg; 0.47 mmol) at room temperature. The mixture was stirred for 1 h at room temperature. The resulting mixture was quenched by ice/water and extracted with EtOAc. The combined organic layers were washed with brine and dried over anhydrous Na₂SO₄. After filtration, the filtrate was concentrated under reduced pressure. The resulting mixture was concentrated under reduced pressure and purified by silica gel column chromatography and eluted with CH₂Cl₂/MeOH (10:1) to afford tert-butyl 7-[2-({4-[(3R)-3-(dimethylamino)pyrrolidine-1-carbonyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate (120.00 mg; 80.0%) as a yellow solid.

(3R)—N,N-dimethyl-1-{4-[(7-{8-methyl-1H,2H,3H-pyrido[2,3-b][1,4]oxazin-7-yl}-5H,6H,7H,8H-pyrido[3,4-d]pyrimidin-2-yl)amino]benzoyl}pyrrolidin-3-amine: A mixture of tert-butyl 7-[2-({4-[(3R)-3-(dimethylamino)pyrrolidine-1-carbonyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate (110.00 mg; 0.16 mmol) and TFA (1.00 mL) in DCM (5.00 mL) was stirred for 1 h at room temperature. The resulting mixture was concentrated under reduced pressure. The residue was purified by Prep-HPLC (Column: XBridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 22% B to 50% B in 9 min., 50% B; Wave Length: 254 nm; RTT (min.): 7) to afford (3R)—N,N-dimethyl-1-{4-[(7-{8-methyl-1H,2H,3H-pyrido[2,3-b][1,4]oxazin-7-yl}-5H,6H,7H,8H-pyrido[3,4-d]pyrimidin-2-yl)amino]benzoyl}pyrrolidin-3-amine (25.70 mg; 31.3%) as a white solid.

HPLC: 98.73% purity, RT=2.57 min. MS: m/z=515.25 [M+H]⁺. ¹H NMR (300 MHz, DMSO-d6): 9.80 (s, 1H), 8.38 (s, 1H), 7.82 (d, J=8.4 Hz, 2H), 7.46 (d, J=8.2 Hz, 2H), 7.29 (s, 1H), 5.53 (s, 1H), 4.19 (t, J=4.4 Hz, 2H), 3.98 (s, 2H), 3.55 (s, 3H), 3.12 (d, J=5.9 Hz, 5H), 2.81 (s, 2H), 2.64 (s, 1H), 2.17 (s, 6H), 2.07 (d, J=17.6 Hz, 4H), 1.70 (d, J=11.1 Hz, 1H).

### Example 43: Synthesis of Compound 43 N2,3-dimethyl-N5-(7-{8-methyl-1H,2H,3H-pyrido[2,3-b][1,4]oxazin-7-yl}-5H,6H,7H,8H-pyrido[3,4-d]pyrimidin-2-yl)-N2-[2-(morpholin-4-yl)ethyl]pyridine-2,5-diamine

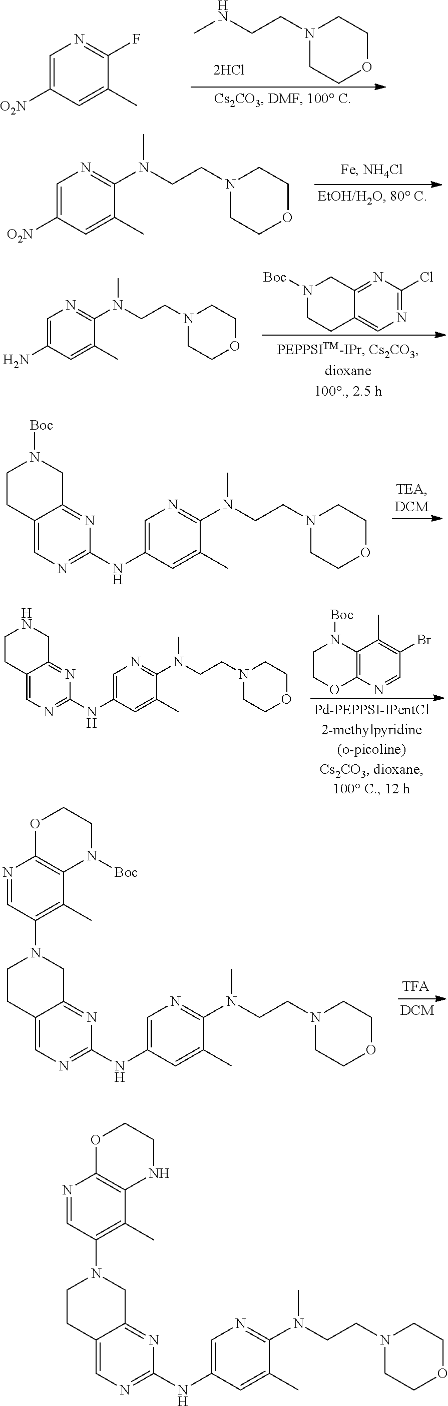

N,3-dimethyl-N-[2-(morpholin-4-yl)ethyl]-5-nitropyridin-2-amine: To a stirred solution of 2-fluoro-3-methyl-5-nitropyridine (635.00 mg; 3.86 mmol) in DMF (6.00 mL) was added methyl[2-(morpholin-4-yl)ethyl]amine (574.50 mg; 3.86 mmol) and Cs₂CO₃ (3975.82 mg; 11.59 mmol) in portions at room temperature under N₂ atmosphere. The resulting mixture was stirred for 1 h at 100° C. under N₂ atmosphere. The resulting mixture was diluted with water (15 mL), extracted with EtOAc (3×10 mL). The combined organic layers were washed with brine (15 mL) and dried over anhydrous Na₂SO₄. The resulting mixture was concentrated under reduced pressure to afford N,3-dimethyl-N-[2-(morpholin-4-yl)ethyl]-5-nitropyridin-2-amine (1.40 g; 80.15%) as a purple oil.

N2,3-dimethyl-N2-[2-(morpholin-4-yl)ethyl]pyridine-2,5-diamine: To a stirred solution of N,3-dimethyl-N-[2-(morpholin-4-yl)ethyl]-5-nitropyridin-2-amine (1.35 g; 4.62 mmol) in EtOH (9.00 mL) were added Fe (1362.64 mg; 23.12 mmol) and NH₄Cl (1301.56 mg; 23.12 mmol, dissolved in H₂O (6.00 mL)) in portions at room temperature under N₂ atmosphere. The resulting mixture was stirred for 1 h at 80° C. under N₂ atmosphere. The resulting mixture was diluted with water (15 mL), extracted with EtOAc (3×10 mL). The combined organic layers were washed with brine (15 mL) and dried over anhydrous Na₂SO₄. The resulting mixture was concentrated under reduced pressure. The residue was purified by silica gel column and eluted with 20% MeOH in DCM to afford N2,3-dimethyl-N2-[2-(morpholin-4-yl)ethyl]pyridine-2,5-diamine (430.00 mg; 35.8%) as a purple oil.

tert-butyl 2-[(5-methyl-6-{methyl[2-(morpholin-4-yl)ethyl]amino}pyridin-3-yl)amino]-5H,6H,7H,8H-pyrido[3,4-d]pyrimidine-7-carboxylate: To a stirred solution of N2,3-dimethyl-N2-[2-(morpholin-4-yl)ethyl]pyridine-2,5-diamine (410.00 mg; 1.57 mmol) in 1,4-dioxane (5.00 mL) were added tert-butyl 2-chloro-5H,6H,7H,8H-pyrido[3,4-d]pyrimidine-7-carboxylate (480.92 mg; 1.73 mmol), PEPPSI™-IPr (138.63 mg; 0.16 mmol) and Cs₂CO₃ (1078.47 mg; 3.14 mmol) in portions at room temperature under N₂ atmosphere. The resulting mixture was stirred for 2.5 h at 100° C. under N₂ atmosphere. The resulting mixture was diluted with water (15 mL), extracted with EtOAc (3×10 mL). The combined organic layers were washed with brine (15 mL) and dried over anhydrous Na₂SO₄. The resulting mixture was concentrated under reduced pressure. The residue was purified by silica gel column and eluted with 10% MeOH in DCM to afford tert-butyl 2-[(5-methyl-6-{methyl[2-(morpholin-4-yl)ethyl]amino}pyridin-3-yl)amino]-5H,6H,7H,8H-pyrido[3,4-d]pyrimidine-7-carboxylate (880.00 mg, 73.9%) as a brown oil.

N2,3-dimethyl-N2-[2-(morpholin-4-yl)ethyl]-N5-{5H,6H,7H,8H-pyrido[3,4-d]pyrimidin-2-yl}pyridine-2,5-diamine: To a stirred solution of tert-butyl 2-[(5-methyl-6-{methyl[2-(morpholin-4-yl)ethyl]amino}pyridin-3-yl)amino]-5H,6H,7H,8H-pyrido[3,4-d]pyrimidine-7-carboxylate (860.00 mg; 1.07 mmol) in DCM (5.00 mL) was added TFA (1.00 mL) in portions at 0° C. under N₂ atmosphere. The resulting mixture was stirred for 1 h at room temperature under N₂ atmosphere. The resulting mixture was diluted with water (15 mL), extracted with DCM (3×10 mL). The combined organic layers were washed with brine (15 mL) and dried over anhydrous Na₂SO₄. The resulting mixture was concentrated under reduced pressure to afford N2,3-dimethyl-N2-[2-(morpholin-4-yl)ethyl]-N5-{5H,6H,7H,8H-pyrido[3,4-d]pyrimidin-2-yl}pyridine-2,5-diamine as a dark brown oil (640.00 mg; 143.1%).

tert-butyl 8-methyl-7-{2-[(5-methyl-6-{methyl[2-(morpholin-4-yl)ethyl]amino}pyridin-3-yl)amino]-5H,6H,7H,8H-pyrido[3,4-d]pyrimidin-7-yl}-1H,2H,3H-pyrido[2,3-b][1,4]oxazine-1-carboxylate: To a stirred solution of N2,3-dimethyl-N2-[2-(morpholin-4-yl)ethyl]-N5-{5H,6H,7H,8H-pyrido[3,4-d]pyrimidin-2-yl}pyridine-2,5-diamine (300.00 mg; 0.70 mmol) in 1,4-dioxane (6.00 mL) were added tert-butyl 7-bromo-8-methyl-1H,2H,3H-pyrido[2,3-b][1,4]oxazine-1-carboxylate (382.04 mg; 1.06 mmol), Pd-PEPPSI-IPentCl 2-methylpyridine (o-picoline) (62.26 mg; 0.07 mmol) and Cs₂CO₃ (482.94 mg; 1.41 mmol) in portions at room temperature under N₂ atmosphere. The resulting mixture was stirred for 2 h at 100° C. under N₂ atmosphere. The resulting mixture was diluted with water (15 mL), extracted with EtOAc (3×10 mL). The combined organic layers were washed with brine (15 mL) and dried over anhydrous Na₂SO₄. The resulting mixture was concentrated under reduced pressure. The residue was purified by silica gel column and eluted with 10% MeOH in DCM to afford tert-butyl 8-methyl-7-{2-[(5-methyl-6-{methyl[2-(morpholin-4-yl)ethyl]amino}pyridin-3-yl)amino]-5H,6H,7H,8H-pyrido[3,4-d]pyrimidin-7-yl}-1H,2H,3H-pyrido[2,3-b][1,4]oxazine-1-carboxylate (290.00 mg, 44.7%) as a dark brown solid.

N2,3-dimethyl-N5-(7-{8-methyl-1H,2H,3H-pyrido[2,3-b][1,4]oxazin-7-yl}-5H,6H,7H,8H-pyrido[3,4-d]pyrimidin-2-yl)-N2-[2-(morpholin-4-yl)ethyl]pyridine-2,5-diamine: To a stirred solution of tert-butyl 8-methyl-7-{2-[(5-methyl-6-{methyl[2-(morpholin-4-yl)ethyl]amino}pyridin-3-yl)amino]-5H,6H,7H,8H-pyrido[3,4-d]pyrimidin-7-yl}-1H,2H,3H-pyrido[2,3-b][1,4]oxazine-1-carboxylate (270.00 mg) in DCM (3.00 mL) was added TFA (0.60 mL) in portions at 0° C. under N₂ atmosphere. The resulting mixture was stirred for 1 h at room temperature under N₂ atmosphere The resulting mixture was added NaHCO₃, then diluted with water (15 mL), extracted with DCM (3×10 mL). The combined organic layers were washed with brine (15 mL) and dried over anhydrous Na₂SO₄. The resulting mixture was concentrated under reduced pressure. The residue was purified by prep-HPLC (Column: XBridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 22% B to 52% B in 9 min., 52% B; Wave Length: 254 nm; RT1 (min.): 7.) to afford N2,3-dimethyl-N5-(7-{8-methyl-1H,2H,3H-pyrido[2,3-b][1,4]oxazin-7-yl}-5H,6H,7H,8H-pyrido[3,4-d]pyrimidin-2-yl)-N₂-[2-(morpholin-4-yl)ethyl]pyridine-2,5-diamine (39.30 mg; Purified Product) as a light yellow solid.

HPLC: 98.05% purity, RT=2.61 min. MS: m/z=532.25 [M+H]⁺. ¹H NMR (400 MHz, DMSO-d6) δ 9.35 (s, 1H), 8.46 (d, J=2.6 Hz, 1H), 8.29 (s, 1H), 7.78 (d, J=2.6 Hz, 1H), 7.28 (s, 1H), 5.52 (t, J=2.7 Hz, 1H), 4.19 (dd, J=5.2, 3.6 Hz, 2H), 3.94 (s, 2H), 3.52 (t, J=4.6 Hz, 4H), 3.34-3.26 (m, 2H), 3.11 (t, J=7.2 Hz, 4H), 2.77 (t, J=5.7 Hz, 2H), 2.54-2.40 (m, 4H), 2.36 (t, J=4.7 Hz, 4H), 2.22 (s, 3H), 2.06 (d, J=15.7 Hz, 3H).

### Example 44: Synthesis of Compound 44 N-(2-methoxyethyl)-4-[(7-{8-methyl-1H,2H,3H-pyrido[2,3-b][1,4]oxazin-7-yl}-5H,6H,7H,8H-pyrido[3,4-d]pyrimidin-2-yl)amino]benzamide

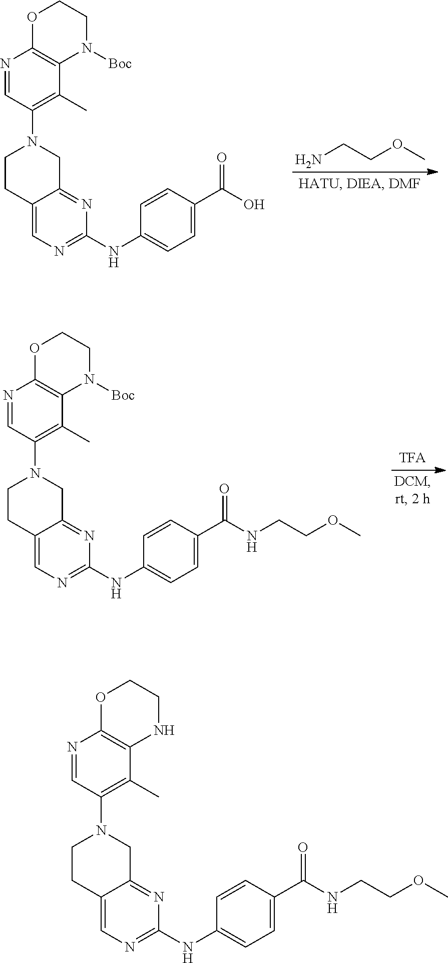

tert-butyl 7-[2-({4-[(2-methoxyethyl)carbamoyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate: To a stirred mixture of 4-[(7-{1-[(tert-butoxy)carbonyl]-8-methyl-1H,2H,3H-pyrido[2,3-b][1,4]oxazin-7-yl}-5H,6H,7H,8H-pyrido[3,4-d]pyrimidin-2-yl)amino]benzoic acid (150.00 mg; 0.24 mmol) and 2-methoxyethan-1-amine (35.37 mg; 0.47 mmol) in DMF (10.00 mL) were added HATU (282.72 mg; 0.71 mmol) and DIEA (64.04 mg; 0.47 mmol) at room temperature. The mixture was stirred for 1 h at room temperature. The resulting mixture was quenched by ice/water and extracted with EtOAc. The combined organic layers were washed with brine and dried over anhydrous Na₂SO₄. After filtration, the filtrate was concentrated under reduced pressure. The resulting mixture was concentrated under reduced pressure and purified by silica gel column chromatography and eluted with CH₂Cl₂/MeOH (10:1) to afford tert-butyl 7-[2-({4-[(2-methoxyethyl)carbamoyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate (190.00 mg; 138.0%) as a yellow oil.

N-(2-methoxyethyl)-4-[(7-{8-methyl-1H,2H,3H-pyrido[2,3-b][1,4]oxazin-7-yl}-5H,6H,7H,8H-pyrido[3,4-d]pyrimidin-2-yl)amino]benzamide: To a stirred mixture of tert-butyl 7-[2-({4-[(2-methoxyethyl)carbamoyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate (300.00 mg; 0.46 mmol) and TFA (3.00 mL) in DCM (15.00 mL) at room temperature. The mixture was stirred for 1 h at room temperature. The resulting mixture was concentrated under reduced pressure. The residue was purified by Prep-HPLC (Column: XBridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 22% B to 50% B in 9 min., 50% B; Wave Length: 254 nm; RT1 (min.): 7) to afford N-(2-methoxyethyl)-4-[(7-{8-methyl-1H,2H,3H-pyrido[2,3-b][1,4]oxazin-7-yl}-5H,6H,7H,8H-pyrido[3,4-d]pyrimidin-2-yl)amino]benzamide (31.80 mg; 14.5%) as a white solid.

HPLC: 99.84% purity, RT=3.18 min. MS: m/z=476.15 [M+H]⁺. ¹H NMR (300 MHz, DMSO-d6): 9.82 (s, 1H), 8.39 (s, 1H), 8.31 (d, J=5.5 Hz, 1H), 7.80 (q, J=9.0 Hz, 4H), 7.29 (s, 1H), 5.53 (s, 1H), 4.19 (t, J=4.2 Hz, 2H), 3.99 (s, 2H), 3.43 (d, J=9.8 Hz, 1H), 3.30 (d, J=3.0 Hz, 1H), 3.27 (s, 3H), 3.12 (d, J=5.8 Hz, 2H), 2.82 (s, 2H), 2.51 (q, J=1.8 Hz, 4H), 2.04 (s, 3H).

### Example 45: Synthesis of Compound 45 2-(dimethylamino)-N-{2-methyl-4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}acetamide

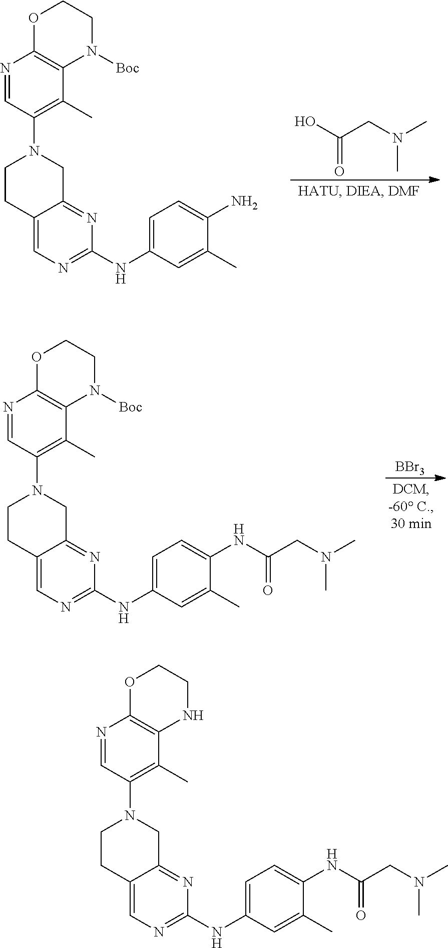

tert-butyl 7-[2-({4-[2-(dimethylamino)acetamido]-3-methylphenyl}amino)-5H,6H,7H,8H-pyrido[3,4-d]pyrimidin-7-yl]-8-methyl-1H,2H,3H-pyrido[2,3-b][1,4]oxazine-1-carboxylate: To a stirred mixture of tert-butyl 7-{2-[(4-amino-3-methylphenyl)amino]-5H,6H,7H,8H-pyrido[3,4-d]pyrimidin-7-yl}-8-methyl-1H,2H,3H-pyrido[2,3-b][1,4]oxazine-1-carboxylate (180.00 mg; 0.36 mmol) and 2-(dimethylamino)acetic acid (62.00 mg; 0.54 mmol) in DMF (10.00 mL) were added HATU (286.00 mg; 0.71 mmol) and DIEA (194.00 mg; 1.43 mmol) in portions at 25° C. The resulting mixture was stirred for 2 h at 25° C. under nitrogen atmosphere. The reaction was diluted with H₂O at 25° C. The resulting mixture was extracted with EA (3×50 mL). The combined organic layers were washed with brine and dried over anhydrous MgSO₄. After filtration, the filtrate was concentrated under reduced pressure. The residue was purified by silica gel column chromatography and eluted with MeOH:DCM=3:97 to afford tert-butyl 7-[2-({4-[2-(dimethylamino)acetamido]-3-methylphenyl}amino)-5H,6H,7H,8H-pyrido[3,4-d]pyrimidin-7-yl]-8-methyl-1H,2H,3H-pyrido[2,3-b][1,4]oxazine-1-carboxylate (150.00 mg, 71.3%) as a yellow solid.

2-(dimethylamino)-N-{2-methyl-4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}acetamide: To a stirred mixture of tert-butyl 7-[2-({4-[2-(dimethylamino)acetamido]-3-methylphenyl}amino)-5H,6H,7H,8H-pyrido[3,4-d]pyrimidin-7-yl]-8-methyl-1H,2H,3H-pyrido[2,3-b][1,4]oxazine-1-carboxylate (120.00 mg; 0.20 mmol) in DCM (4.00 mL) was added BBr₃ in DCM (0.61 mL; 0.61 mmol) dropwise at −60° C. The resulting mixture was stirred for 1 h at −60° C. under nitrogen atmosphere. The reaction was quenched with NaHCO₃ (aq.) at −20° C. The resulting mixture was extracted with EA (3×50 mL). The combined organic layers were washed with brine and dried over anhydrous MgSO₄. After filtration, the filtrate was concentrated under reduced pressure. The crude product was purified by Prep-HPLC with the following conditions (Column: XBridge Prep OBD C18 Column, 30*150 mm, 5 μm; Mobile Phase A: water (10 mmol/L NH₄HCO₃+0.1% NH₄OH), Mobile Phase B: ACN; Flow rate: 60 mL/min.; Gradient: 25% B to 52% B in 8 min., 52% B; Wave Length: 254 nm; RT1 (min.): 7) to afford 2-(dimethylamino)-N-{2-methyl-4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}acetamide (21.80 mg; 21.3%) as a yellow solid.

HPLC: 97.28% purity, RT=2.49 min. MS: m/z=489.15 [M+H]⁺. ¹H NMR (400 MHz, DMSO-d6) δ 9.46 (s, 1H), 9.38 (s, 1H), 8.32 (s, 1H), 7.58 (d, J=7.2 Hz, 2H), 7.36-7.30 (m, 1H), 7.28 (s, 1H), 5.54 (d, J=2.8 Hz, 1H), 4.19 (t, J=4.4 Hz, 2H), 3.95 (s, 2H), 3.36 (s, 2H), 3.30 (p, J=3.0 Hz, 2H), 3.12 (t, J=5.7 Hz, 2H), 2.79 (d, J=5.7 Hz, 2H), 2.47 (s, 6H), 2.16 (s, 3H), 2.04 (s, 3H).

### Example 46: Synthesis of Compound 46 7-{8-methyl-1H,2H,3H-pyrido[2,3-b][1,4]oxazin-7-yl}-N-{3-[(methylamino)methyl]phenyl}-5H,6H,7H,8H-pyrido[3,4-d]pyrimidin-2-amine

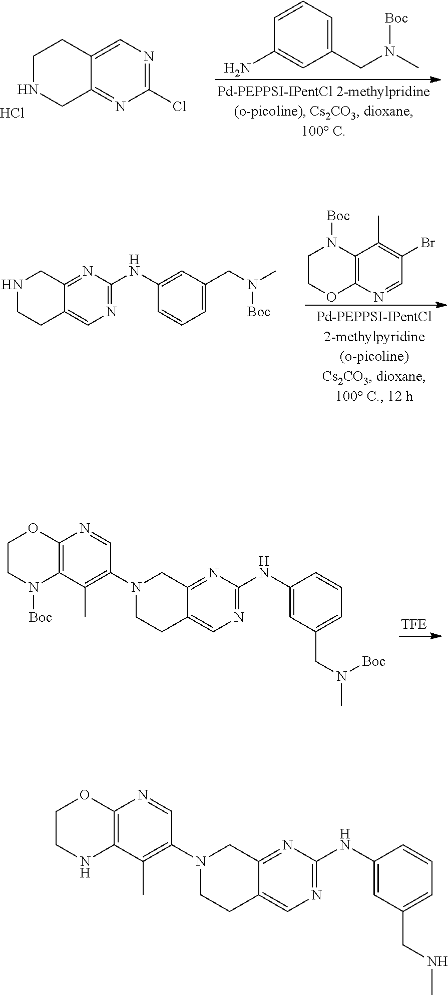

tert-butyl N-methyl-N-{[3-({5H,6H,7H,8H-pyrido[3,4-d]pyrimidin-2-yl}amino)phenyl]methyl}carbamate: To a solution of 2-chloro-5H,6H,7H,8H-pyrido[3,4-d]pyrimidine hydrochloride (400.00 mg; 1.73 mmol) and tert-butyl N-[(3-aminophenyl)methyl]-N-methylcarbamate (431.00 mg; 1.73 mmol) in 1,4-dioxane (20.00 mL) were added Pd-PEPPSI-IPentCl 2-methylpyridine (o-picoline) (153.00 mg; 0.17 mmol) and Cs₂CO₃ (1187.00 mg; 3.46 mmol). The resulting mixture was stirred for 2 h at 100° C. under nitrogen atmosphere. The resulting mixture was concentrated under reduced pressure. The residue was purified by silica gel column chromatography and eluted with DCM/MeOH (5:1) to afford tert-butyl N-methyl-N-{[3-({5H,6H,7H,8H-pyrido[3,4-d]pyrimidin-2-yl}amino)phenyl]methyl}carbamate (240.00 mg 32.9%) as a yellow solid.

tert-butyl 7-(2-{[3-({[(tert-butoxy)carbonyl](methyl)amino}methyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate: To a stirred mixture of tert-butyl N-methyl-N-{[3-({5H,6H,7H,8H-pyrido[3,4-d]pyrimidin-2-yl}amino)phenyl]methyl}carbamate (240.00 mg; 0.57 mmol) and Pd-PEPPSI-IPentCl 2-methylpyridine (o-picoline) (51.00 mg; 0.06 mmol) in 1,4-dioxane (10.00 mL) were added tert-butyl 7-bromo-8-methyl-1H,2H,3H-pyrido[2,3-b][1,4]oxazine-1-carboxylate (192.00 mg; 0.57 mmol) and Cs₂CO₃ (375.00 mg; 1.14 mmol) in portions at 25° C. The resulting mixture was stirred for 2 h at 100° C. under nitrogen atmosphere. The residue was purified by silica gel column chromatography and eluted with PE:EA=1:1 to afford tert-butyl 7-(2-{[3-({[(tert-butoxy)carbonyl](methyl)amino}methyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (140.00 mg; 38.9%) as a yellow solid.

7-{8-methyl-1H,2H,3H-pyrido[2,3-b][1,4]oxazin-7-yl}-N-{3-[(methylamino)methyl]phenyl}-5H,6H,7H,8H-pyrido[3,4-d]pyrimidin-2-amine: A solution of tert-butyl7-(2-{[3-({[(tert-butoxy)carbonyl](methyl)amino}methyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (120.00 mg; 0.19 mmol) in TFE (5.00 mL) was stirred for overnight at 120 degrees C. under nitrogen atmosphere. The mixture was basified to pH [7] with saturated NaHCO₃ (aq.). The resulting mixture was extracted with DCM. The combined organic layers were washed with brine and dried over anhydrous Na₂SO₄. After filtration, the filtrate was concentrated under reduced pressure. The residue was purified by Prep-HPLC with the following conditions (Column: Xselect CSH C18 OBD Column 30*150 mm, 5 μm, n; Mobile Phase A: water (0.1% FA), Mobile Phase B: ACN; Flow rate: 60 mL/min.; Gradient: 5% B to 30% B in 8 min., 30% B; Wave Length: 254/220 nm; RTT (min.): 6.7) to afford 7-{8-methyl-1H,2H,3H-pyrido[2,3-b][1,4]oxazin-7-yl}-N-{3-[(methylamino)methyl]phenyl}-5H,6H,7H,8H-pyrido[3,4-d]pyrimidin-2-amine (1.60 mg; 2.0%) as a yellow solid.

HPLC: 98.79% purity, RT=2.66 min. MS: m/z=418.00 [M+H]⁺. ¹H NMR (300 MHz, DMSO-d6) δ 9.54 (s, 1H), 8.34 (s, 1H), 8.30 (s, 1H), 7.76 (s, 1H), 7.72-7.63 (m, 1H), 7.28 (d, J=5.5 Hz, 1H), 7.23 (d, J=7.9 Hz, 1H), 6.95 (d, J=7.5 Hz, 1H), 5.54 (s, 1H), 4.20 (t, J=4.4 Hz, 2H), 3.97 (s, 2H), 3.80 (s, 2H), 3.31 (d, J=4.0 Hz, 2H), 3.16-3.10 (m, 2H), 2.81 (d, J=5.7 Hz, 2H), 2.40 (s, 3H), 2.05 (s, 3H).

### Example 47: Synthesis of Compound 47 6-{8-methyl-1H,2H,3H-pyrido[2,3-b][1,4]oxazin-7-yl}-N-(2-methyl-2,3-dihydro-1H-isoindol-5-yl)-5,6,7,8-tetrahydro-2,6-naphthyridin-3-amine

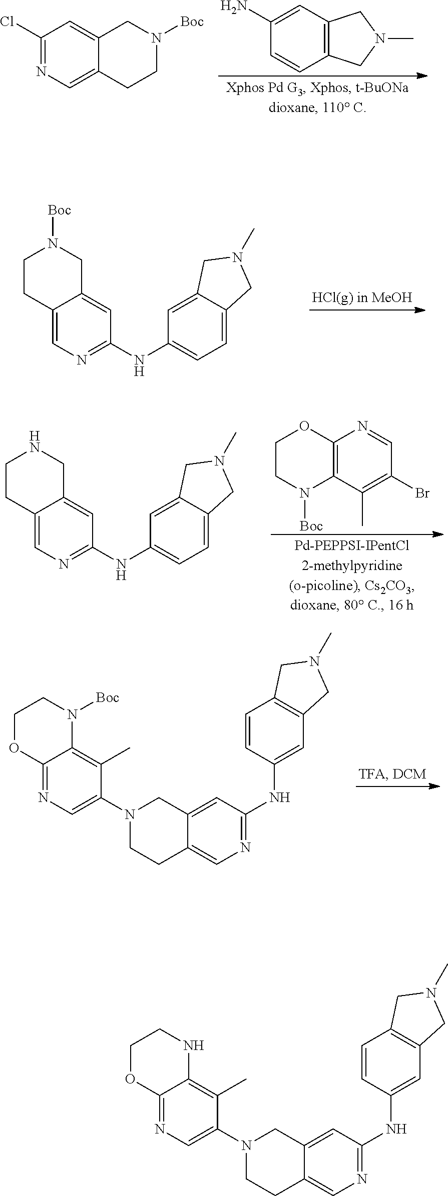

tert-butyl 7-[(2-methyl-2,3-dihydro-1H-isoindol-5-yl)amino]-1,2,3,4-tetrahydro-2,6-naphthyridine-2-carboxylate: To a stirred solution of tert-butyl 7-chloro-1,2,3,4-tetrahydro-2,6-naphthyridine-2-carboxylate (250.00 mg; 0.91 mmol) and 2-methyl-2,3-dihydro-1H-isoindol-5-amine (137.87 mg; 0.91 mmol) in 1,4-dioxane (10.00 mL) was added Pd-PEPPSI-IPentCl 2-methylpyridine (o-picoline) (80.62 mg; 0.09 mmol) and Cs₂CO₃ (625.34 mg; 1.82 mmol). The resulting mixture was stirred for 3 h at 100° C. under nitrogen atmosphere. The resulting mixture was concentrated under reduced pressure. The residue was purified by silica gel column chromatography and eluted with CH₂Cl₂/MeOH (5:1) to afford tert-butyl 7-[(2-methyl-2,3-dihydro-1H-isoindol-5-yl) amino]-1,2,3,4-tetrahydro-2,6-naphthyridine-2-carboxylate (330.00 mg; 91.88%) as a yellow solid.

N-(2-methyl-2,3-dihydro-1H-isoindol-5-yl)-5,6,7,8-tetrahydro-2,6-naphthyridin-3-amine: A solution of tert-butyl 7-[(2-methyl-2,3-dihydro-1H-isoindol-5-yl)amino]-1,2,3,4-tetrahydro-2,6-naphthyridine-2-carboxylate (310.00 mg; 0.79 mmol) in HCl (g) in MeOH (6.00 mL) was stirred for 1 h at room temperature. The resulting mixture was concentrated under reduced pressure to afford N-(2-methyl-2,3-dihydro-1H-isoindol-5-yl)-5,6,7,8-tetrahydro-2,6-naphthyridin-3-amine (274.00 mg, 84.46%, crude product) as a blue solid.

tert-butyl 8-methyl-7-{7-[(2-methyl-2,3-dihydro-1H-isoindol-5-yl)amino]-1,2,3,4-tetrahydro-2,6-naphthyridin-2-yl}-1H,2H,3H-pyrido[2,3-b][1,4]oxazine-1-carboxylate: To a stirred solution of N-(2-methyl-2,3-dihydro-1H-isoindol-5-yl)-5,6,7,8-tetrahydro-2,6-naphthyridin-3-amine (200.00 mg; 0.49 mmol) and tert-butyl 7-bromo-8-methyl-1H,2H,3H-pyrido[2,3-b][1,4]oxazine-1-carboxylate (167.95 mg; 0.49 mmol) in 1,4-dioxane (10.00 mL) was added Pd-PEPPSI-IPentCl 2-methylpyridine (o-picoline) (42.90 mg; 0.05 mmol) and Cs₂CO₃ (332.77 mg; 1.47 mmol). The resulting mixture was stirred for 3 h at 100° C. under nitrogen atmosphere. The resulting mixture was concentrated under reduced pressure. The residue was purified by silica gel column chromatography and eluted with CH₂Cl₂/MeOH (5:1) to afford tert-butyl 8-methyl-7-{7-[(2-methyl-2,3-dihydro-1H-isoindol-5-yl)amino]-1,2,3,4-tetrahydro-2,6-naphthyridin-2-yl}-1H,2H,3H-pyrido[2,3-b][1,4]oxazine-1-carboxylate (90.00 mg; 34.33%) as a yellow solid.

6-{8-methyl-1H,2H,3H-pyrido[2,3-b][1,4]oxazin-7-yl}-N-(2-methyl-2,3-dihydro-1H-isoindol-5-yl)-5,6,7,8-tetrahydro-2,6-naphthyridin-3-amine: To a stirred solution of tert-butyl 8-methyl-7-{7-[(2-methyl-2,3-dihydro-1H-isoindol-5-yl)amino]-1,2,3,4-tetrahydro-2,6-naphthyridin-2-yl}-1H,2H,3H-pyrido[2,3-b][1,4]oxazine-1-carboxylate (90.00 mg; 0.17 mmol) in DCM (5.00 mL) was added TFA (1.00 mL) in portions at room temperature. After stirring for 1 h at room temperature, the resulting mixture was concentrated under reduced pressure. The crude product was purified by Prep-HPLC (Column: XBridge Prep OBD C18 Column, 30*150 mm, 5 μm; Mobile Phase A: water (10 mmol/L NH₄HCO₃+0.1% NH₄OH), Mobile Phase B: ACN; Flow rate: 60 mL/min.; Gradient: 25% B to 52% B in 8 min., 52% B; Wave Length: 254 nm; RT1 (min.): 7) to afford 6-{8-methyl-1H,2H,3H-pyrido[2,3-b][1,4]oxazin-7-yl}-N-(2-methyl-2,3-dihydro-1H-isoindol-5-yl)-5,6,7,8-tetrahydro-2,6-naphthyridin-3-amine (16.40 mg; 21.83%) as a white solid.

HPLC: 95.00% purity, RT=2.60 min. MS: m/z=429.15 [M+H]⁺. ¹H NMR (300 MHz, DMSO-d6): 8.64 (s, 1H), 7.86 (s, 1H), 7.44 (d, J=2.0 Hz, 1H), 7.21 (dd, J=8.1, 2.0 Hz, 1H), 7.14 (s, 1H), 6.95 (d, J=8.2 Hz, 1H), 6.45 (s, 1H), 5.39 (d, J=2.7 Hz, 1H), 4.07 (t, J=4.3 Hz, 2H), 3.86 (s, 2H), 3.65 (d, J=11.1 Hz, 4H), 3.17 (d, J=5.6 Hz, 2H), 2.97 (t, J=5.7 Hz, 2H), 2.69 (t, J=5.6 Hz, 2H), 2.36 (s, 3H), 1.91 (s, 3H).

### Example 48: Synthesis of Compound 48 N-{2-methyl-4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}-2-(morpholin-4-yl)acetamide

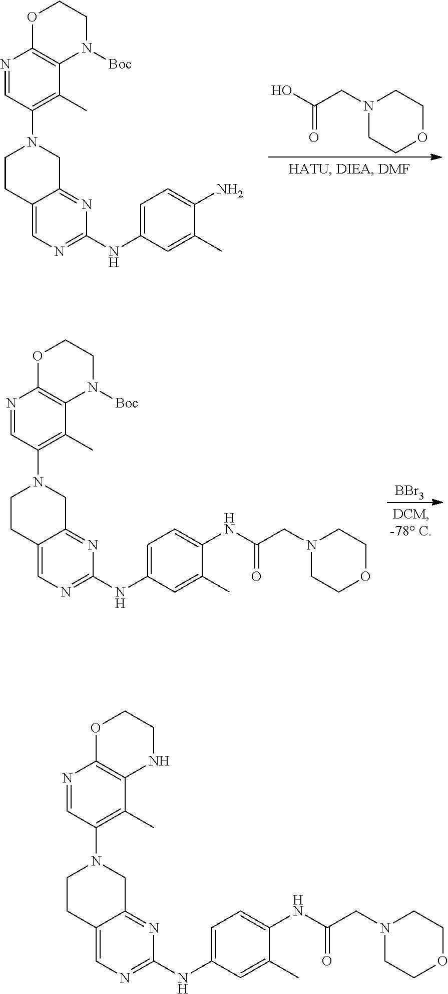 [SMILES: Cc1cc(Nc2ncc3c(n2)CN(c2cnc4c(c2C)N(C(=O)OC(C)(C)C)CCO4)CC3)ccc1N ; O=C(O)CN1CCOCC1 ; Cc1cc(Nc2ncc3c(n2)CN(c2cnc4c(c2C)N(C(=O)OC(C)(C)C)CCO4)CC3)ccc1NC(=O)CN1CCOCC1 ; "BBr3" ; Cc1cc(Nc2ncc3c(n2)CN(c2cnc4c(c2C)NCCO4)CC3)ccc1NC(=O)CN1CCOCC1]

tert-butyl 8-methyl-7-[2-({3-methyl-4-[2-(morpholin-4-yl)acetamido]phenyl}amino)-5H,6H,7H,8H-pyrido[3,4-d]pyrimidin-7-yl]-1H,2H,3H-pyrido[2,3-b][1,4]oxazine-1-carboxylate: To a stirred mixture of tert-butyl 7-{2-[(4-amino-3-methylphenyl)amino]-5H,6H,7H,8H-pyrido[3,4-d]pyrimidin-7-yl}-8-methyl-1H,2H,3H-pyrido[2,3-b][1,4]oxazine-1-carboxylate (180.00 mg; 0.36 mmol) and 2-(morpholin-4-yl)acetic acid (0.01 mL; 0.54 mmol) in DMF (10.00 mL) were added HATU (286.00 mg; 0.71 mmol) and DIEA (197.00 mg; 1.45 mmol) in portions at 25° C. The resulting mixture was stirred for 2 h at 25° C. under nitrogen atmosphere. The reaction was diluted with H₂O at 25° C. The resulting mixture was extracted with EA (3×50 mL). The combined organic layers were washed with brine and dried over anhydrous MgSO₄. After filtration, the filtrate was concentrated under reduced pressure. The residue was purified by silica gel column chromatography and eluted with MeOH:DCM=3:97 to afford tert-butyl 8-methyl-7-[2-({3-methyl-4-[2-(morpholin-4-yl) acetamido]phenyl}amino)-5H,6H,7H,8H-pyrido[3,4-d]pyrimidin-7-yl]-1H,2H,3H-pyrido[2,3-b][1,4]oxazine-1-carboxylate (200.00 mg; 88.7%) as a yellow solid.

N-{2-methyl-4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}-2-(morpholin-4-yl)acetamide: To a stirred mixture of tert-butyl 8-methyl-7-[2-({3-methyl-4-[2-(morpholin-4-yl)acetamido]phenyl}amino)-5H,6H,7H,8H-pyrido[3,4-d]pyrimidin-7-yl]-1H,2H,3H-pyrido[2,3-b][1,4]oxazine-1-carboxylate (30.00 mg; 0.05 mmol) in DCM (2.00 mL) was added BBr₃ in DCM (0.14 mL; 0.14 mmol) in portions at −70° C. The resulting mixture was stirred for 1 h at −70° C. under nitrogen atmosphere. The reaction was quenched with Na₂CO₃ (aq.) at −20° C. The resulting mixture was extracted with EA (3×100 mL). The combined organic layers were washed with brine and dried over anhydrous MgSO₄. After filtration, the filtrate was concentrated under reduced pressure. The crude product was purified by Prep-HPLC with the following conditions (Column: XBridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 20% B to 50% B in 8 min., 50% B; Wave Length: 254 nm; RT1 (min.): 7) to afford N-{2-methyl-4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}-2-(morpholin-4-yl)acetamide (21.80 mg; 85.2%) as a yellow solid.

HPLC: 98.65% purity, RT=2.69 min. MS: m/z=531.35 [M+H]⁺. ¹H NMR (400 MHz, DMSO-d6) δ 9.43 (s, 1H), 9.24 (s, 1H), 8.32 (s, 1H), 7.58 (d, J=9.3 Hz, 2H), 7.47 (d, J=8.4 Hz, 1H), 7.28 (s, 1H), 5.53 (d, J=3.5 Hz, 1H), 4.19 (t, J=4.4 Hz, 2H), 3.95 (s, 2H), 3.66 (t, J=4.5 Hz, 4H), 3.29 (d, J=6.1 Hz, 2H), 3.11 (d, J=4.8 Hz, 4H), 2.78 (t, J=5.7 Hz, 2H), 2.55 (t, J=4.5 Hz, 4H), 2.18 (s, 3H), 2.04 (s, 3H).

### Example 49: Synthesis of Compound 49 7-(8-methyl-2,3-dihydro-1H-pyrido[2,3-b][1,4]oxazin-7-yl)-N-(2-((methylsulfonyl)methyl)pyridin-4-yl)-5,6,7,8-tetrahydropyrido[3,4-d]pyrimidin-2-amine

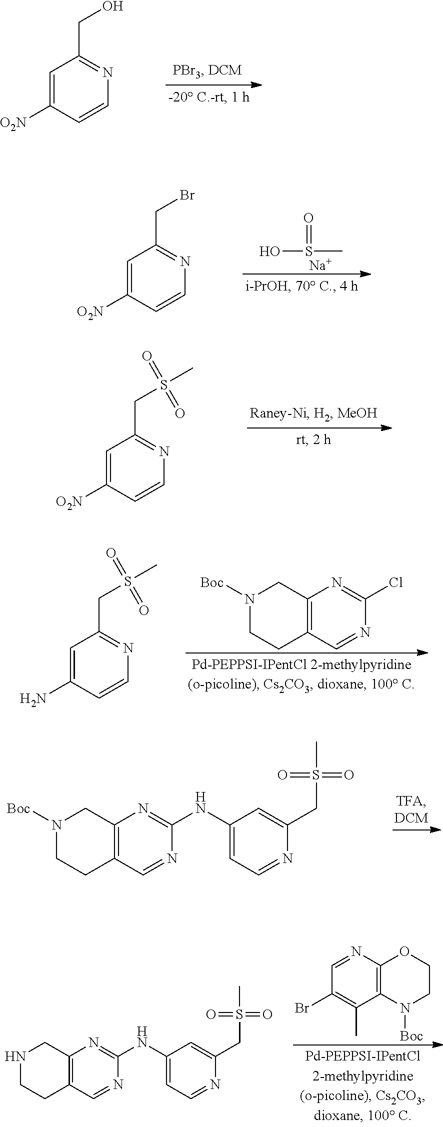

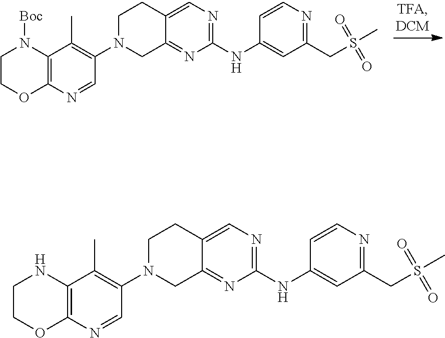 [SMILES: Cc1c(N2CCc3cnc(Nc4ccnc(CS(C)(=O)=O)c4)nc3C2)cnc2c1N(C(=O)OC(C)(C)C)CCO2 ; Cc1c(N2CCc3cnc(Nc4ccnc(CS(C)(=O)=O)c4)nc3C2)cnc2c1NCCO2]

2-(bromomethyl)-4-nitropyridine: To a solution of (4-nitropyridin-2-yl) methanol (2.00 g; 12.72 mmol) in DCM (100.00 mL) was added PBr₃ (1.64 mL; 16.53 mmol) at −20° C. The resulting mixture was stirred for 2 h at room temperature. The reaction was quenched with sat. NaHCO₃ (aq.) (20 mL) at 0° C. The resulting mixture was extracted with DCM (3×30 mL). The combined organic layers were dried over anhydrous Na₂SO₄. After filtration, the filtrate was concentrated under reduced pressure. The residue was purified by silica gel column chromatography and eluted with PE. This resulted in 2-(bromomethyl)-4-nitropyridine (1.80 g; 65.03%) as a brown solid.

2-((methylsulfonyl)methyl)-4-nitropyridine: To a stirred solution of 2-(bromomethyl)-4-nitropyridine (1.80 g; 8.27 mmol) in i-PrOH (20.00 mL) was added sodium methanesulfinate (4.26 g; 41.35 mmol). The resulting mixture was stirred for 4 h at 70° C. The mixture was allowed to cool down to room temperature and was concentrated under reduced pressure. The residue was purified by silica gel column chromatography and eluted with PE/EtOAc (1:2) to afford 2-(methanesulfonylmethyl)-4-nitropyridine (1.00 g; 54.69%) as a white solid.

2-((methylsulfonyl)methyl) pyridin-4-amine: To a solution of 2-(methanesulfonylmethyl)-4-nitropyridine (1.00 g; 4.28 mmol) in MeOH (20.00 mL) was added Raney-Ni (1.10 g; 1.29 mmol) at room temperature. The mixture stirring at room temperature for 2 h under H₂. The resulting mixture was filtered and the filter cake was washed with MeOH (50 mL). The filtrate was concentrated under reduced pressure. This resulted in 2-((methylsulfonyl)methyl) pyridin-4-amine (0.60 g, crude product; 74.94%) as a white solid.

tert-butyl 2-((2-((methylsulfonyl)methyl) pyridin-4-yl) amino)-5,8-dihydropyrido[3,4-d]pyrimidine-7(6H)-carboxylate: To a stirred mixture of 2-(methanesulfonylmethyl)pyridin-4-amine (310.98 mg; 1.67 mmol; 1.50 eq.) and tert-butyl 2-chloro-5H,6H,7H,8H-pyrido[3,4-d]pyrimidine-7-carboxylate (300.00 mg; 1.11 mmol; 1.00 eq.) in t-BuOH (18.00 mL) and H₂O (1.50 mL) were added Pd₂(dba)₃ (107.21 mg; 0.11 mmol; 0.10 eq.), X-PHOS (111.63 mg; 0.22 mmol; 0.20 eq.) and Cs₂CO₃ (762.92 mg; 2.22 mmol; 2.00 eq.) in portions at 25 degrees C. The resulting mixture was stirred for 2 h at 100 degrees C. under argon atmosphere. The resulting mixture was concentrated under vacuum. The residue was purified by silica gel column chromatography and eluted with MeOH:DCM=1:9 to afford tert-butyl 2-((2-((methylsulfonyl)methyl)pyridin-4-yl)amino)-5,8-dihydropyrido[3,4-d]pyrimidine-7(6H)-carboxylate (310.00 mg; 94.53%) as a yellow solid.

N-(2-((methylsulfonyl)methyl) pyridin-4-yl)-5,6,7,8-tetrahydropyrido[3,4-d]pyrimidin-2-amine: To a stirred mixture of tert-butyl 7-{[6-(methanesulfonylmethyl) pyridin-3-yl]amino}-1,2,3,4-tetrahydro-2,6-naphthyridine-2-carboxylate (300.00 mg; 0.68 mmol) in DCM (6.00 mL) and was added TFA (1.00 mL) at room temperature. After stirring for 1 h at room temperature, the resulting mixture was quenched with water (15 mL) and basified to pH=with 10. Then mixture was extracted with CH₂Cl₂ (3*15 mL). The combined organic layers were washed with brine (30 mL) and dried over anhydrous Na₂SO₄. After filtration, the filtrate was concentrated under reduced pressure to afford N-[6-(methanesulfonylmethyl) pyridin-3-yl]-5,6,7,8-tetrahydro-2,6-naphthyridin-3-amine (150 mg, crude product) as a yellow solid.

tert-butyl 8-methyl-7-(2-((2-((methylsulfonyl)methyl)pyridin-4-yl)amino)-5,8-dihydropyrido[3,4-d]pyrimidin-7(6H)-yl)-2,3-dihydro-1H-pyrido[2,3-b][1,4]oxazine-1-carboxylate: To a solution of 2-(methanesulfonylmethyl)-N-{5H,6H,7H,8H-pyrido[3,4-d]pyrimidin-2-yl}pyridin-4-amine (110.00 mg; 0.31 mmol) and tert-butyl 7-bromo-8-methyl-1H,2H,3H-pyrido[2,3-b][1,4]oxazine-1-carboxylate (156.75 mg; 0.47 mmol) in 1,4-dioxane (10.00 mL) were added Cs₂CO₃ (322.94 mg; 0.94 mmol) and Pd-PEPPSI-IPentCl 2-methylpyridine (o-picoline) (27.76 mg; 0.03 mmol). The resulting mixture was stirred for 3 h at 100° C. under nitrogen atmosphere. The mixture was concentrated under reduced pressure. The residue was purified by silica gel column chromatography and eluted with CH₂Cl₂/MeOH (11:1) to afford tert-butyl 7-(2-{[2-(methanesulfonylmethyl)pyridin-4-yl]amino}-5H,6H,7H,8H-pyrido[3,4-d]pyrimidin-7-yl)-8-methyl-1H,2H,3H-pyrido[2,3-b][1,4]oxazine-1-carboxylate (110 mg, 66.00%) as a yellow solid.

7-(8-methyl-2,3-dihydro-1H-pyrido[2,3-b][1,4]oxazin-7-yl)-N-(2-((methylsulfonyl)methyl)pyridin-4-yl)-5,6,7,8-tetrahydropyrido[3,4-d]pyrimidin-2-amine: To a stirred mixture of tert-butyl 7-(2-{[2-(methanesulfonylmethyl)pyridin-4-yl]amino}-5H,6H,7H,8H-pyrido[3,4-d]pyrimidin-7-yl)-8-methyl-1H,2H,3H-pyrido[2,3-b][1,4]oxazine-1-carboxylate (100.00 mg; 0.12 mmol) in DCM (4.00 mL) and was added TFA (1.00 mL) at room temperature. After stirring for 1 h at room temperature, the mixture was concentrated under reduced pressure. The crude product was purified by Prep-HPLC (Column: XBridge Prep OBD C18 Column, 30*150 mm, 5?m; Mobile Phase A: water (10 mmol/L NH₄HCO₃+0.05% NH₄OH), Mobile Phase B: ACN; Flow rate: 60 mL/min.; Gradient: 20% B to 46% B in 8 min., 46% B; Wave Length: 254 nm; RTT (min.): 7) to afford 2-(methanesulfonylmethyl)-N-(7-{8-methyl-1H,2H,3H-pyrido[2,3-b][1,4]oxazin-7-yl}-5H,6H,7H,8H-pyrido[3,4-d]pyrimidin-2-yl)pyridin-4-amine (15.60 mg; 99.10%) as an off-white solid.

HPLC: 99.1% purity, RT=2.210 min. MS: m/z=468.2 [M+H]⁺. ¹H NMR (300 MHz, DMSO-d6): 10.70 (d, J=46.4 Hz, 1H), 8.42 (d, J=5.4 Hz, 1H), 8.34 (t, J=8.0 Hz, 1H), 8.03 (s, 1H), 7.84 (d, J=7.1 Hz, 1H), 7.20 (s, 1H), 5.54 (s, 1H), 4.63 (d, J=18.7 Hz, 2H), 4.10 (q, J=3.8 Hz, 2H), 3.96 (d, J=3.4 Hz, 2H), 3.21 (d, J=4.5 Hz, 2H), 3.11-2.98 (m, 3H), 2.78 (d, J=6.7 Hz, 2H), 2.48-2.39 (m, 2H), 1.94 (d, J=2.0 Hz, 3H).

### Example 50: Synthesis of Compound 50 7-(8-methyl-2,3-dihydro-1H-pyrido[2,3-b][1,4]oxazin-7-yl)-N-(3-((methylsulfonyl) methyl)phenyl)-5,6,7,8-tetrahydropyrido[3,4-d]pyrimidin-2-amine

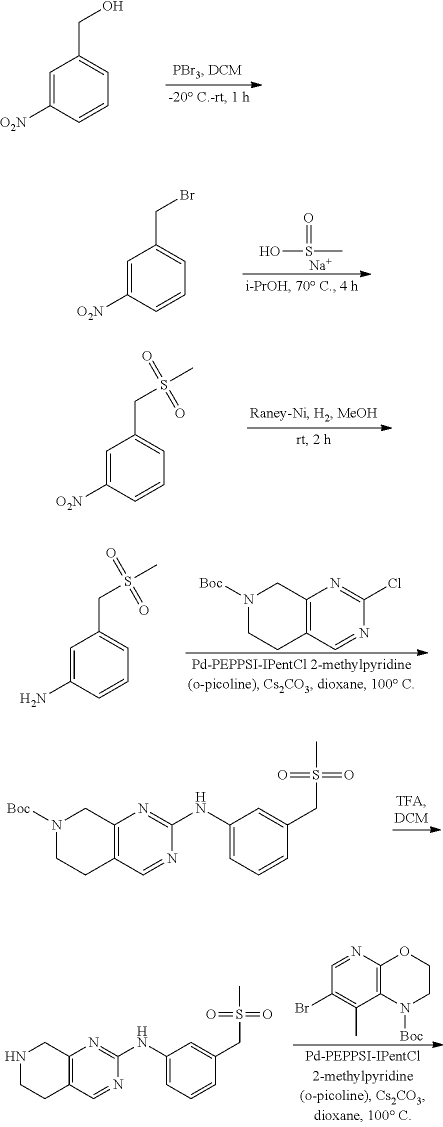

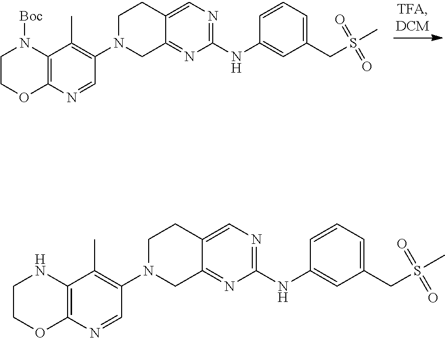 [SMILES: Cc1c(N2CCc3cnc(Nc4cccc(CS(C)(=O)=O)c4)nc3C2)cnc2c1N(C(=O)OC(C)(C)C)CCO2 ; Cc1c(N2CCc3cnc(Nc4cccc(CS(C)(=O)=O)c4)nc3C2)cnc2c1NCCO2]

tert-butyl 2-((3-((methylsulfonyl)methyl)phenyl) amino)-5,8-dihydropyrido[3,4-d]pyrimidine-7(6H)-carboxylate: To a stirred mixture of tert-butyl 2-chloro-5H,6H,7H,8H-pyrido[3,4-d]pyrimidine-7-carboxylate (300.00 mg; 1.11 mmol) and 3-(methanesulfonylmethyl)aniline (231.26 mg; 1.22 mmol) in 1,4-dioxane (10.00 mL) were added PEPPSI-IPr (72.41 mg; 0.11 mmol) and Cs₂CO₃ (762.92 mg; 2.22 mmol) at room temperature. The mixture was stirred for 2 h at 100° C. under argon atmosphere. The resulting mixture was concentrated under reduced pressure and purified by silica gel column chromatography and eluted with PE/EtOAc (10:1) to afford [0008] tert-butyl 2-((3-((methylsulfonyl)methyl)phenyl) amino)-5,8-dihydropyrido[3,4-d]pyrimidine-7(6H)-carboxylate (500.00 mg; 98.2%) as a yellow oil.

N-(3-((methylsulfonyl) methyl)phenyl)-5,6,7,8-tetrahydropyrido[3,4-d]pyrimidin-2-amine: To a stirred mixture of tert-butyl 2-{[3-(methanesulfonylmethyl)phenyl]amino}-5H,6H,7H,8H-pyrido[3,4-d]pyrimidine-7-carboxylate (540.00 mg) in DCM (15.00 mL) was added TFA (3.00 mL) at room temperature. The mixture was stirred for 1 h at room temperature. The mixture was basified to pH=9 with NaHCO₃ (aq.). The resulting mixture was quenched by ice/water and extracted with EtOAc. The combined organic layers were washed with brine and dried over anhydrous Na₂SO₄. After filtration, the filtrate was concentrated under reduced pressure. This resulted in N-(3-((methylsulfonyl) methyl) phenyl)-5,6,7,8-tetrahydropyrido[3,4-d]pyrimidin-2-amine (340.00 mg; 96.57%) as a yellow solid.

tert-butyl 8-methyl-7-(2-((3-((methylsulfonyl)methyl)phenyl)amino)-5,8-dihydropyrido[3,4-d]pyrimidin-7(6H)-yl)-2,3-dihydro-1H-pyrido[2,3-b][1,4]oxazine-1-carboxylate: To a stirred mixture of tert-butyl 7-bromo-8-methyl-1H,2H,3H-pyrido[2,3-b][1,4]oxazine-1-carboxylate (626.17 mg; 1.90 mmol) and N-[3-(methanesulfonylmethyl)phenyl]-5H,6H,7H,8H-pyrido[3,4-d]pyrimidin-2-amine (309.00 mg; 0.95 mmol) in 1,4-dioxane (10.00 mL) were added Pd-PEPPSI-IPentCl 2-methylpyridine (o-picoline) (84.11 mg; 0.10 mmol) and Cs₂CO₃ (652.38 mg; 1.90 mmol) at room temperature. The mixture was stirred for 12 h at 100° C. under argon atmosphere. The resulting mixture was concentrated under reduced pressure and purified by silica gel column chromatography and eluted with PE/EtOAc (1:1) to afford tert-butyl 8-methyl-7-(2-((3-((methylsulfonyl) methyl)phenyl) amino)-5,8-dihydropyrido[3,4-d]pyrimidin-7(6H)-yl)-2,3-dihydro-TH-pyrido[2,3-b][1,4]oxazine-1-carboxylate (100.00 mg; 18.4%) as a yellow oil.

7-(8-methyl-2,3-dihydro-1H-pyrido[2,3-b][1,4]oxazin-7-yl)-N-(3-((methylsulfonyl) methyl)phenyl)-5,6,7,8-tetrahydropyrido[3,4-d]pyrimidin-2-amine: To a stirred mixture of tert-butyl 7-(2-{[3-(methanesulfonylmethyl) phenyl]amino}-5H,6H,7H,8H-pyrido[3,4-d]pyrimidin-7-yl)-8-methyl-1H,2H,3H-pyrido[2,3-b][1,4]oxazine-1-carboxylate (80.00 mg; 0.13 mmol) in 2,2,2-trifluoroethan-1-ol (5.00 mL) at room temperature. The mixture was stirred for 2 h at 120° C. The resulting mixture was concentrated under reduced pressure. The residue was purified by Prep-HPLC (Column: XBridge Prep OBD C18 Column, 30*150 mm, 5 μm; Mobile Phase A: water (10 mmol/L NH₄HCO₃+0.1% NH₄OH), Mobile Phase B: ACN; Flow rate: 60 mL/min.; Gradient: 22% B to 50% B in 9 min., 50% B; Wavelength: 254 nm; RTT (min.): 7). This resulted in N-[3-(methanesulfonylmethyl) phenyl]-7-{8-methyl-1H,2H,3H-pyrido[2,3-b][1,4]oxazin-7-yl}-5H,6H,7H,8H-pyrido[3,4-d]pyrimidin-2-amine (25.60 mg, 40.5%) as a white solid.

HPLC: 99.0% purity, RT=3.10 min. MS: m/z=467.0 [M+H]⁺. ¹H NMR (300 MHz, DMSO-d6): 9.49 (s, 1H), 8.22 (s, 1H), 7.75 (s, 1H), 7.57 (d, J=8.7 Hz, 1H), 7.21-7.10 (m, 2H), 6.84 (d, J=7.6 Hz, 1H), 5.42 (s, 1H), 4.30 (s, 2H), 4.07 (s, 2H), 3.84 (s, 2H), 3.32 (s, 2H), 3.00 (d, J=6.0 Hz, 2H), 2.82 (s, 3H), 2.68 (s, 2H), 1.92 (s, 3H).

### Example 51: Synthesis of Compound 51 6-(8-methyl-2,3-dihydro-1H-pyrido[2,3-b][1,4]oxazin-7-yl)-N-(6-((methylsulfonyl) methyl) pyridin-3-yl)-5,6,7,8-tetrahydro-2,6-naphthyridin-3-amine

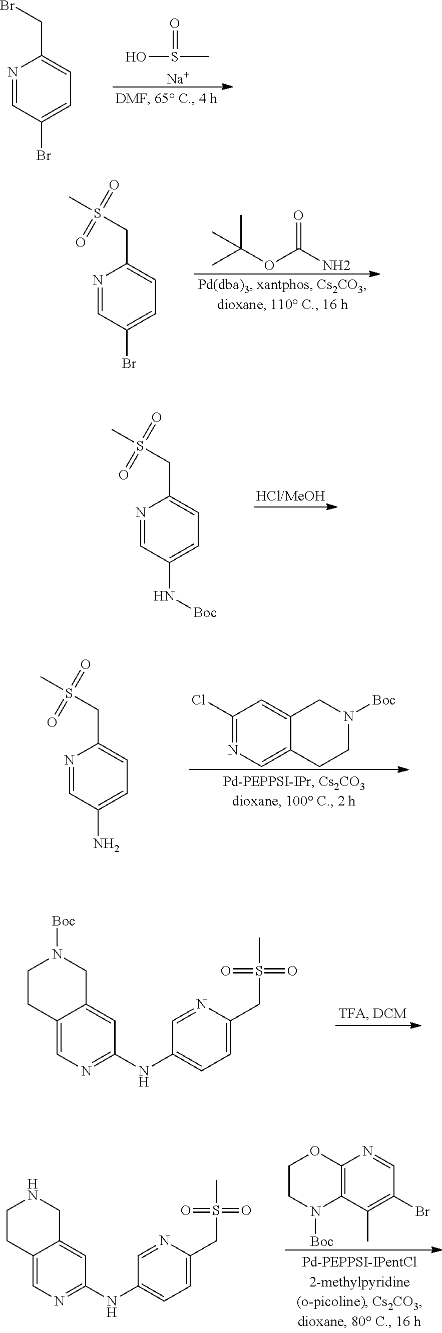

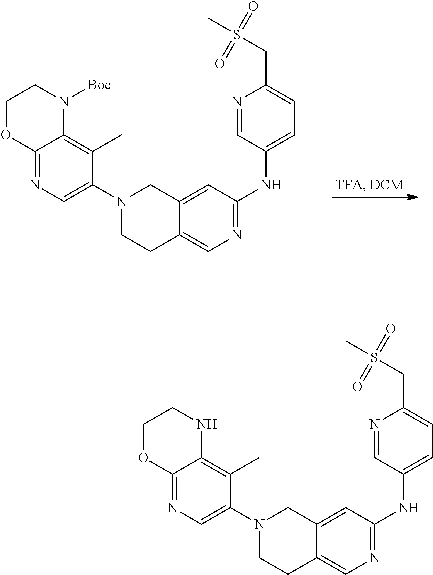 [SMILES: Cc1c(N2CCc3cnc(Nc4ccc(CS(C)(=O)=O)nc4)cc3C2)cnc2c1N(C(=O)OC(C)(C)C)CCO2 ; Cc1c(N2CCc3cnc(Nc4ccc(CS(C)(=O)=O)nc4)cc3C2)cnc2c1NCCO2]

5-bromo-2-((methylsulfonyl) methyl)pyridine: To a stirred solution of 5-bromo-2-(bromomethyl)pyridine (100.00 mg; 0.39 mmol) in DMF (1.50 mL) was added sodium methanesulfinate (201.38 mg; 1.95 mmol). The resulting mixture was stirred for 4 h at 65° C. under nitrogen atmosphere. The mixture was allowed to cool down to room temperature. After filtration, and filter cake washed with DCM (50 mL), the filtrate was concentrated under reduced pressure to afford 5-bromo-2-(methanesulfonylmethyl)pyridine (90.00 mg; 80.45%) as a brown solid.

tert-butyl (6-((methylsulfonyl) methyl) pyridin-3-yl) carbamate: To a stirred mixture of 5-bromo-2-(methanesulfonylmethyl)pyridine (500.00 mg; 1.95 mmol) and tert-butyl carbamate (360.93 mg; 2.93 mmol) in 1,4-dioxane (15.00 mL) was added Pd₂(dba)₃ (188.09 mg; 0.20 mmol), Xantphos (228.09 mg; 0.39 mmol) and Cs₂CO₃ (1338.47 mg; 3.90 mmol) at room temperature. After stirring for 16 h at 90° C. under argon atmosphere. The mixture was concentrated under reduced pressure. The residue was purified by silica gel column chromatography and eluted with PE/EtOAc (4:1) to afford tert-butyl N-[6-(methanesulfonylmethyl) pyridin-3-yl]carbamate (300.00 mg; 50.48%) as a yellow solid.

6-((methylsulfonyl) methylpyridin-3-amine: To a stirred mixture of tert-butyl N-[6-(methanesulfonylmethyl) pyridin-3-yl] carbamate (290.00 mg; 0.95 mmol) in DCM (6.00 mL) and was added TFA (1.00 mL) at room temperature. After stirring for 1 h at room temperature, the resulting mixture was quenched with water (15 mL) and basified to pH=10 with Na₂CO₃. Then mixture was extracted with CH₂Cl₂ (3*15 mL). The combined organic layers were washed with brine (30 mL) and dried over anhydrous Na₂SO₄. After filtration, the filtrate was concentrated under reduced pressure to afford 6-(methanesulfonylmethyl) pyridin-3-amine (175.00 mg; 98.60%) as a yellow solid.

tert-butyl 7-((6-((methylsulfonyl)methyl) pyridin-3-yl) amino)-3,4-dihydro-2,6-naphthyridine-2(1H)-carboxylate: To a stirred mixture of 6-(methanesulfonylmethyl) pyridin-3-amine (168.24 mg; 0.90 mmol) and tert-butyl 7-chloro-1,2,3,4-tetrahydro-2,6-naphthyridine-2-carboxylate (165.00 mg; 0.60 mmol) in 1,4-dioxane (10.00 mL) were added PEPPSI-IPr (39.17 mg; 0.06 mmol; 0.10 eq.) and Cs₂CO₃ (412.73 mg; 1.20 mmol) in portions at 25° C. The resulting mixture was stirred for 2 h at 100° C. under argon atmosphere. The resulting mixture was concentrated under reduced pressure. The residue was purified by silica gel column chromatography and eluted with CH₂Cl₂/MeOH (10:1) to afford tert-butyl 7-{[6-(methanesulfonylmethyl)pyridin-3-yl]amino}-1,2,3,4-tetrahydro-2,6-naphthyridine-2-carboxylate (270.00 mg; 92.52%) as a brown solid.

N-(6-((methylsulfonyl) methyl) pyridin-3-yl)-5,6,7,8-tetrahydro-2,6-naphthyridin-3-amine: To a stirred mixture of tert-butyl 7-{[6-(methanesulfonylmethyl) pyridin-3-yl]amino}-1,2,3,4-tetrahydro-2,6-naphthyridine-2-carboxylate (270.00 mg; 0.56 mmol) in DCM (6.00 mL) and was added TFA (1.00 mL) at room temperature. After stirring for 1 h at room temperature, the resulting mixture was quenched with water (15 mL) and basified to pH=10. Then mixture was extracted with CH₂Cl₂ (3*15 mL). The combined organic layers were washed with brine (30 mL) and dried over anhydrous Na₂SO₄. After filtration, the filtrate was concentrated under reduced pressure to afford N-[6-(methanesulfonylmethyl) pyridin-3-yl]-5,6,7,8-tetrahydro-2,6-naphthyridin-3-amine (110.00 mg; 53.12%) as a brown solid.

tert-butyl 8-methyl-7-(7-((6-((methylsulfonyl)methyl)pyridin-3-yl)amino)-3,4-dihydro-2,6-naphthyridin-2(1H)-yl)-2,3-dihydro-1H-pyrido[2,3-b][1,4]oxazine-1-carboxylate: To a solution of N-[6-(methanesulfonylmethyl)pyridin-3-yl]-5,6,7,8-tetrahydro-2,6-naphthyridin-3-amine (110.00 mg; 0.30 mmol) and tert-butyl 7-bromo-8-methyl-1H,2H,3H-pyrido[2,3-b][1,4]oxazine-1-carboxylate (147.68 mg; 0.44 mmol) in 1,4-dioxane (10.00 mL) were added Cs₂CO₃ (304.25 mg; 0.89 mmol) and Pd-PEPPSI-IPentCl 2-methylpyridine (o-picoline) (26.15 mg; 0.03 mmol). The resulting mixture was stirred for 3 h at 100° C. under nitrogen atmosphere. The mixture was concentrated under reduced pressure. The residue was purified by silica gel column chromatography and eluted with CH₂Cl₂/MeOH (11:1) to afford tert-butyl 7-(7-{[6-(methanesulfonylmethyl)pyridin-3-yl]amino}-1,2,3,4-tetrahydro-2,6-naphthyridin-2-yl)-8-methyl-1H,2H,3H-pyrido[2,3-b][1,4]oxazine-1-carboxylate (105.00 mg; 28.96%) as a yellow solid.

6-(8-methyl-2,3-dihydro-1H-pyrido[2,3-b][1,4]oxazin-7-yl)-N-(6-((methylsulfonyl) methyl) pyridin-3-yl)-5,6,7,8-tetrahydro-2,6-naphthyridin-3-amine: To a stirred mixture of tert-butyl 7-(7-{[6-(methanesulfonylmethyl) pyridin-3-yl]amino}-1,2,3,4-tetrahydro-2,6-naphthyridin-2-yl)-8-methyl-1H,2H,3H-pyrido[2,3-b][1,4]oxazine-1-carboxylate (95.00 mg; 0.08 mmol) in DCM (4.00 mL) and was added TFA (1.00 mL) at room temperature. After stirring for 1 h at room temperature the mixture was concentrated under reduced pressure. The crude product was purified by Prep-HPLC (Column: XBridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 20% B to 48% B in 9 min., 48% B; Wave Length: 254 nm; RT1 (min.): 7; Number Of Runs: 2) to afford N-[6-(methanesulfonylmethyl)pyridin-3-yl]-6-{8-methyl-1H,2H,3H-pyrido[2,3-b][1,4]oxazin-7-yl}-5,6,7,8-tetrahydro-2,6-naphthyridin-3-amine (12.80 mg; 33.67%) as a light yellow solid.

HPLC: 95.0% purity, RT=2.14 min. MS: m/z=467.2 [M+H]⁺. ¹H NMR (400 MHz, DMSO-d6): 9.70 (s, 1H), 8.89 (s, 1H), 8.23 (s, 1H), 8.09 (s, 1H), 7.48 (d, J=8.6 Hz, 1H), 7.33 (s, 1H), 6.76 (s, 1H), 4.60 (s, 2H), 4.42 (s, 2H), 4.11 (s, 2H), 3.43 (s, 2H), 3.14 (s, 2H), 3.01 (s, 3H), 2.89 (s, 2H), 2.16 (s, 3H).

### Example 52: Synthesis of Compound 52 1-methyl-4-(4-((6-(8-methyl-2,3-dihydro-1H-pyrido[2,3-b][1,4]oxazin-7-yl)-5,6,7,8-tetrahydro-2,6-naphthyridin-3-yl) amino)phenyl) piperidin-4-ol

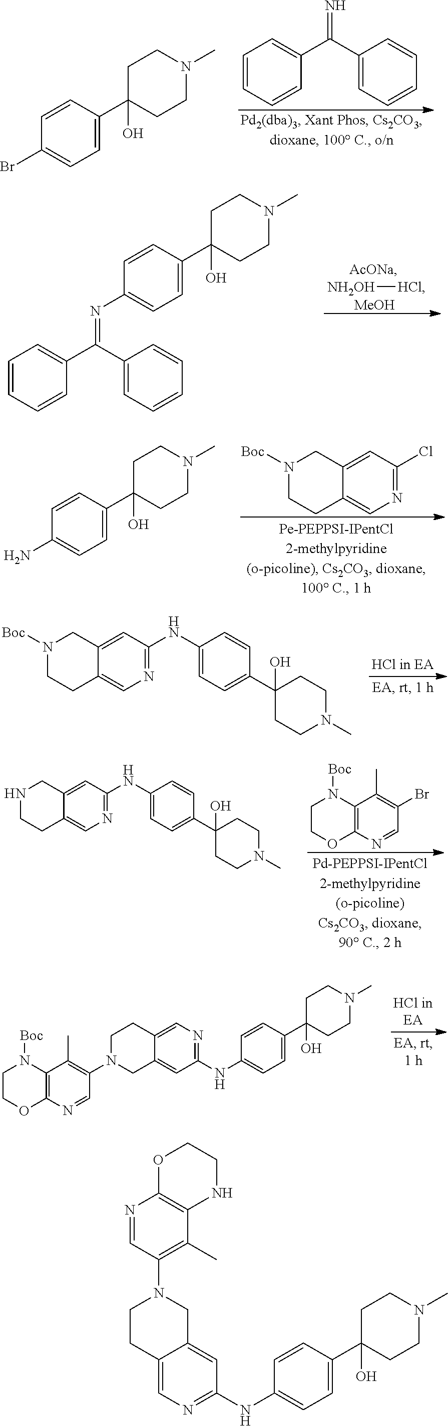

4-(4-((diphenylmethylene)amino)phenyl)-1-methylpiperidin-4-ol: To a stirred solution of 4-(4-bromophenyl)-1-methylpiperidin-4-ol (1.60 g; 5.74 mmol) and diphenylmethanimine (1.32 g; 6.89 mmol) in 1,4-dioxane (20.00 mL) were added Xantphos (42.00 mg; 0.07 mmol), Pd₂(dba)₃ (73.00 mg; 0.07 mmol; 0.01 eq.) and cesium carbonate (5.91 g; 17.23 mmol) at room temperature under N₂ atmosphere. The resulting mixture was stirred for overnight at 100° C. under N₂ atmosphere. The resulting mixture was poured into water, the mixture was extracted with EtOAc (100 mL×3). The combined organic layers were washed with brine dried over anhydrous Na₂SO₄ and concentrated under reduced pressure. The residue was purified by silica gel column and eluted with DCM/MeOH (0.2 M NH₄OH)=5:1 to afford 4-{4-[(diphenylmethylidene)amino]phenyl}-1-methylpiperidin-4-ol (2.00 g; 93.1%) as an orange solid.

4-(4-aminophenyl)-1-methylpiperidin-4-ol: To a stirred solution of 4-{4-[(diphenylmethylidene)amino]phenyl}-1-methylpiperidin-4-ol (1.98 g; 5.30 mmol) in methanol (20.00 mL) were added sodium acetate trihydrate (2.18 g; 15.89 mmol; 3.00 eq.) and hydroxylamine hydrochloride (0.77 g; 10.59 mmol; 2.00 eq.) in portions at room temperature under N₂ atmosphere. The resulting mixture was stirred for 1 h at room temperature under N₂ atmosphere. The resulting mixture was concentrated under reduced pressure. The residue was purified by silica gel column and eluted with DCM:MeOH (0.2 M NH₄OH)=5:1 to afford 4-(4-aminophenyl)-1-methylpiperidin-4-ol (1.00 g; 85.3%) as a yellow solid.

tert-butyl 7-((4-(4-hydroxy-1-methylpiperidin-4-yl)phenyl)amino)-3,4-dihydro-2,6-naphthyridine-2(1H)-carboxylate: To a stirred solution of 4-(4-aminophenyl)-1-methylpiperidin-4-ol (282.00 mg; 1.27 mmol) and tert-butyl 7-chloro-1,2,3,4-tetrahydro-2,6-naphthyridine-2-carboxylate (300.00 mg; 1.06 mmol) in 1,4-dioxane (5.00 mL) were added Pd-PEPPSI-IPentCl 2-methylpyridine (o-picoline) (94.00 mg; 0.11 mmol) and cesium carbonate (1.09 g; 3.18 mmol) in portions at room temperature under N₂ atmosphere. The resulting mixture was stirred for 1 h at 100° C. under N₂ atmosphere. The mixture was allowed to cool down to room temperature. The resulting mixture was poured into water, the mixture was extracted with EtOAc (50 mL×3). The combined organic layers were washed with brine dried over anhydrous Na₂SO₄ and concentrated under reduced pressure. The residue was purified by reverse flash chromatography with the following conditions: column, C18 silica gel; mobile phase, ACN in water (0.05% NH₄HCO₃), 65% to 75% gradient in 30 min.; detector, UV 254 nm, to afford tert-butyl 8-methyl-7-(7-{[2-(morpholin-4-yl)pyridin-4-yl]amino}-1,2,3,4-tetrahydro-2,6-naphthyridin-2-yl)-1H,2H,3H-pyrido[2,3-b][1,4]oxazine-1-carboxylate (170.00 mg; 39.5%) as a light yellow solid.

1-methyl-4-(4-((5,6,7,8-tetrahydro-2,6-naphthyridin-3-yl) amino) phenyl) piperidin-4-ol: To a stirred solution of tert-butyl 7-{[4-(4-hydroxy-1-methylpiperidin-4-yl)phenyl]amino}-1,2,3,4-tetrahydro-2,6-naphthyridine-2-carboxylate (350.00 mg; 0.79 mmol) in ethyl acetate (4.00 mL) was added hydrochloric acid (4.0 M solution in ethyl acetate) (1.00 mL; 4.00 mmol) dropwise at 0° C. The resulting mixture was stirred for 1 h at room temperature under N₂ atmosphere. The precipitated solids were collected by filtration and washed with PE (3×2 mL). This resulted in 1-methyl-4-{4-[(5,6,7,8-tetrahydro-2,6-naphthyridin-3-yl) amino]phenyl}piperidin-4-ol (260.00 mg; 70.2%) as a yellow solid.

tert-butyl 7-(7-((4-(4-hydroxy-1-methylpiperidin-4-yl)phenyl)amino)-3,4-dihydro-2,6-naphthyridin-2(1H)-yl)-8-methyl-2,3-dihydro-1H-pyrido[2,3-b][1,4]oxazine-1-carboxylate: To a stirred solution of 1-methyl-4-{4-[(5,6,7,8-tetrahydro-2,6-naphthyridin-3-yl)amino]phenyl}piperidin-4-ol (260.00 mg; 0.55 mmol) and tert-butyl 7-bromo-8-methyl-1H,2H,3H-pyrido[2,3-b][1,4]oxazine-1-carboxylate (228.00 mg; 0.66 mmol) in 1,4-dioxane (3.00 mL) were added cesium carbonate (569.00 mg; 1.66 mmol) and Pd-PEPPSI-IPentCl 2-methylpyridine (o-picoline) (49.00 mg; 0.06 mmol) at room temperature under N₂ atmosphere. The resulting mixture was stirred for overnight at 100° C. under N₂ atmosphere. The mixture was allowed to cool down to room temperature. The resulting mixture was poured into water, the mixture was extracted with EtOAc (50 mL×3). The combined organic layers were washed with brine dried over anhydrous Na₂SO₄ and concentrated under reduced pressure. The residue was purified by silica gel column chromatography and eluted with DCM:MeOH (10:1) to afford tert-butyl 7-(7-{[4-(4-hydroxy-1-methylpiperidin-4-yl)phenyl]amino}-1,2,3,4-tetrahydro-2,6-naphthyridin-2-yl)-8-methyl-1H,2H,3H-pyrid o[2,3-b][1,4]oxazine-1-carboxylate (180.00 mg; 43.2%) as a light yellow solid.

1-methyl-4-(4-((6-(8-methyl-2,3-dihydro-1H-pyrido[2,3-b][1,4]oxazin-7-yl)-5,6,7,8-tetrahydro-2,6-naphthyridin-3-yl)amino)phenyl)piperidin-4-ol: To a stirred solution of tert-butyl 7-(7-{[4-(4-hydroxy-1-methylpiperidin-4-yl)phenyl]amino}-1,2,3,4-tetrahydro-2,6-naphthyridin-2-yl)-8-methyl-1H,2H,3H-pyrido[2,3-b][1,4]oxazine-1-carboxylate (150.00 mg; 0.20 mmol) in dichloromethane (4.00 mL) was added hydrochloric acid 4.0 M solution in ethyl acetate (1.00 mL; 2.00 mmol) dropwise at 0° C. under N₂ atmosphere. The resulting mixture was stirred for 1 h at room temperature under N₂ The precipitated solids were collected by filtration and washed with PE (3×2 mL). The crude product was purified by Prep-HPLC with the following conditions (Column: X Bridge Prep OBD C18 Column, 30*150 mm; Mobile Phase A: water (10 mmol/L NH₄HCO₃+0.05% NH₄OH), Mobile Phase B: ACN; Flow rate: 60 mL/min.; Gradient: 15% to 45% in 8 min.; Wave Length: 254 nm; RT1 (min.): 7; Number Of Runs: 1) to afford 1-methyl-4-{4-[(6-{8-methyl-1H,2H,3H-pyrido[2,3-b][1,4]oxazin-7-yl}-5,6,7,8-tetrahydro-2,6-naphthyridin-3-yl) amino]phenyl}piperidin-4-ol (8.40 mg; 8.2%) as a light yellow solid.

HPLC: 95.0% purity, RT=3.19 min. MS: m/z=487.3 [M+H]⁺. ¹H NMR (300 MHz, Methanol-d4) 7.92 (s, 1H), 7.45-7.32 (m, 4H), 7.25 (s, 1H), 6.61 (s, 1H), 4.29 (dd, J=5.1, 3.8 Hz, 2H), 4.01 (s, 2H), 3.41 (t, J=4.4 Hz, 2H), 3.17 (t, J=5.8 Hz, 2H), 2.88 (t, J=5.7 Hz, 2H), 2.76 (d, J=11.5 Hz, 2H), 2.59 (t, J=11.6 Hz, 2H), 2.36 (s, 3H), 2.13 (s, 3H), 2.19-2.03 (m, 2H), 1.77 (d, J=13.6 Hz, 2H).

### Example 53: Synthesis of Compound 53 Synthesis of compound 7-(8-methyl-2,3-dihydro-1H-pyrido[2,3-b][1,4]oxazin-7-yl)-N-(4-((methylamino)methyl) phenyl)-5,6,7,8-tetrahydropyrido[3,4-d]pyrimidin-2-amine

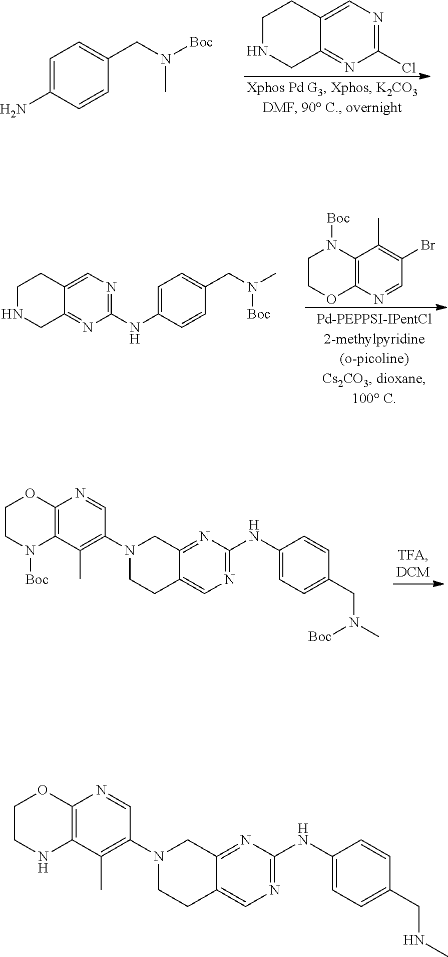

tert-butyl methyl(4-((5,6,7,8-tetrahydropyrido[3,4-d]pyrimidin-2-yl) amino) benzyl) carbamate: To a solution of 2-chloro-5H,6H,7H,8H-pyrido[3,4-d]pyrimidine hydrochloride (400.00 mg; 1.54 mmol) and tert-butyl N-[(4-aminophenyl)methyl]-N-methylcarbamate (460.00 mg; 1.85 mmol) in DMF (5.00 mL) were added Xphos Pd G₃ (138.00 mg; 0.15 mmol), Xphos (155.00 mg; 0.31 mmol; 0.20 eq.) and K₂CO₃ (894.00 mg; 6.16 mmol). The resulting mixture was stirred for overnight at 90° C. under nitrogen atmosphere. The resulting mixture was concentrated under reduced pressure. The residue was purified by silica gel column chromatography and eluted with DCM/MeOH (6:1) to afford tert-butyl N-methyl-N-{[4-({5H,6H,7H,8H-pyrido[3,4-d]pyrimidin-2-yl}amino)phenyl] methyl}carbamate (160.00 mg; 25.7%) as a yellow solid.

tert-butyl 7-(2-((4-(((tert-butoxycarbonyl) (methyl)amino)methyl)phenyl)amino)-5,8-dihydropyrido[3,4-d]pyrimidin-7(6H)-yl)-8-methyl-2,3-dihydro-1H-pyrido[2,3-b][1,4]oxazine-1-carboxylate: To a solution of tert-butyl N-methyl-N-{[4-({5H,6H,7H,8H-pyrido[3,4-d]pyrimidin-2-yl}amino)phenyl]methyl}carbamate (140.00 mg; 0.35 mmol) and tert-butyl 7-bromo-8-methyl-1H,2H,3H-pyrido[2,3-b][1,4]oxazine-1-carboxylate (139.00 mg; 0.42 mmol) in 1,4-dioxane (2.00 mL) were added and cesium carbonate (238.00 mg; 0.69 mmol). The resulting mixture was stirred for 3 h at 100° C. under nitrogen atmosphere. The resulting mixture was concentrated under reduced pressure. The residue was purified by silica gel column chromatography and eluted with PE/EA (1:9) to afford tert-butyl 7-(2-{[4-({[(tert-butoxy) carbonyl] (methyl)amino}methyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (100.00 mg; 45.7%) as a yellow solid.

7-(8-methyl-2,3-dihydro-1H-pyrido[2,3-b][1,4]oxazin-7-yl)-N-(4-((methylamino) methyl)phenyl)-5,6,7,8-tetrahydropyrido[3,4-d]pyrimidin-2-amine: To a stirred solution of tert-butyl 7-(2-{[4-({[(tert-butoxy) carbonyl](methyl)amino}methyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (90.00 mg) in DCM (2.00 mL) was added TFA (0.50 mL) in portions at room temperature. The resulting mixture was stirred for 3 h at room temperature under nitrogen atmosphere. The mixture was basified to pH [7] with saturated NaHCO₃ (aq.). The resulting mixture was extracted with DCM. The combined organic layers were washed with brine and dried over anhydrous Na₂SO₄. After filtration, the filtrate was concentrated under reduced pressure. The residue was purified by Prep-HPLC with the following conditions (Column: Xselect CSH C18 OBD Column 30*150 mm, 5 μm, n; Mobile Phase A: water (0.1% FA), Mobile Phase B: ACN; Flow rate: 60 mL/min.; Gradient: 3% B to 20% B in 8 min., 20% B; Wave Length: 254/220 nm; RT1 (min.): 6.8; Number Of Runs: 2) to afford 7-{8-methyl-1H,2H,3H-pyrido[2,3-b][1,4]oxazin-7-yl}-N-{4-[(methylamino)methyl]phenyl}-5H,6H,7H,8H-pyrido[3,4-d]pyrimidin-2-amine (3.90 mg; 0.05%) as a yellow solid.

HPLC: 97.6% purity, RT=2.29 min. MS: m/z=418.0 [M+H]⁺. ¹H NMR (300 MHz, DMSO-d6) δ 9.42 (s, 1H), 8.21 (s, 2H), 7.59 (d, J=8.3 Hz, 2H), 7.21-7.06 (m, 3H), 5.42 (s, 1H), 4.07 (t, J=4.3 Hz, 2H), 3.84 (s, 2H), 3.59 (s, 2H), 3.18 (s, 2H), 2.99 (d, J=5.9 Hz, 2H), 2.64 (d, J=18.4 Hz, 2H), 2.22 (s, 3H), 1.92 (s, 3H).

### Example 54: Synthesis of Compound 54 6-(8-methyl-2,3-dihydro-1H-pyrido[2,3-b][1,4]oxazin-7-yl)-N-(2-morpholinopyridin-4-yl)-5,6,7,8-tetrahydro-2,6-naphthyridin-3-amine

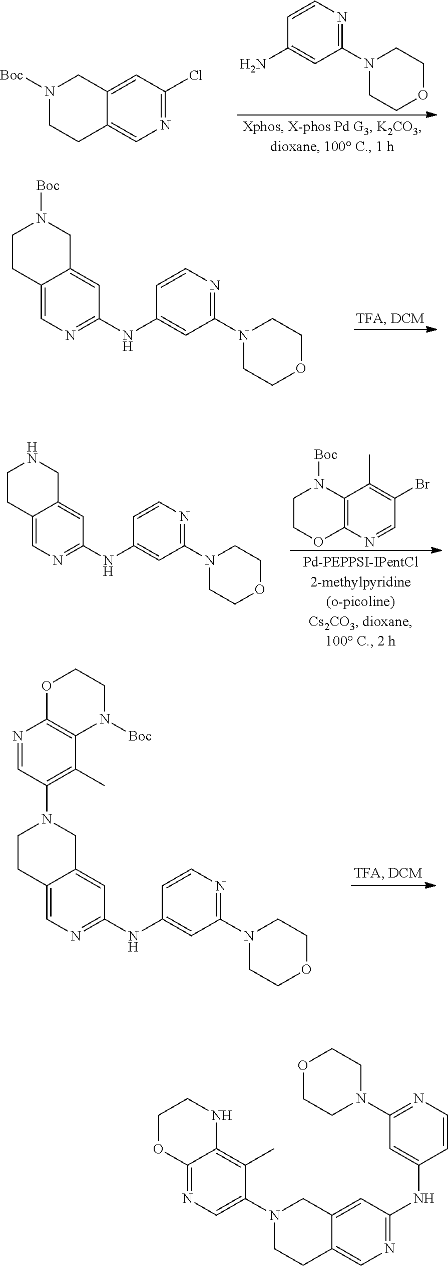

tert-butyl 7-((2-morpholinopyridin-4-yl) amino)-3,4-dihydro-2,6-naphthyridine-2(1H)-carboxylate: To a stirred solution of tert-butyl 7-chloro-1,2,3,4-tetrahydro-2,6-naphthyridine-2-carboxylate (300.00 mg; 1.06 mmol) and 2-(morpholin-4-yl)pyridin-4-amine (220.00 mg; 1.17 mmol) in 1,4-dioxane (3.00 mL) were added K₂CO₃ (463.00 mg; 3.18 mmol), X-PHOS (52.00 mg; 0.10 mmol) and Xphos Pd G3 (94.00 mg; 0.11 mmol) in portions at room temperature under N₂ atmosphere. The resulting mixture was stirred for 1 h at 100° C. under N₂ atmosphere. The resulting mixture was poured into water, the mixture was extracted with EtOAc (50 mL×3). The combined organic layers were washed with brine dried over anhydrous Na₂SO₄ and concentrated under reduced pressure. The residue was purified by silica gel column and eluted with DCM:MeOH=10:1 to afford tert-butyl 7-{[2-(morpholin-4-yl) pyridin-4-yl]amino}-1,2,3,4-tetrahydro-2,6-naphthyridine-2-carboxylate (400.00 mg; 84.9%) as a light yellow oil.

N-(2-morpholinopyridin-4-yl)-5,6,7,8-tetrahydro-2,6-naphthyridin-3-amine: To a stirred solution of tert-butyl 7-{[2-(morpholin-4-yl) pyridin-4-yl]amino}-1,2,3,4-tetrahydro-2,6-naphthyridine-2-carboxylate (380.00 mg; 0.86 mmol) in DCM (3.00 mL) was added TFA (1.00 mL) at room temperature under N₂ atmosphere. The resulting mixture was stirred for 1 h at room temperature under N₂ atmosphere. The mixture was allowed to cool down to room temperature. The resulting mixture was concentrated under reduced pressure. The residue was purified by reverse flash chromatography with the following conditions: column, C18 silica gel; mobile phase, ACN in water (0.05% NH₄HCO₃), 30% to 50% gradient in 30 min.; detector, UV 254 nm, to afford N-[2-(morpholin-4-yl) pyridin-4-yl]-5,6,7,8-tetrahy dro-2,6-naphthyridin-3-amine (250.00 mg; 93.1%) as a light yellow solid.

tert-butyl 8-methyl-7-(7-((2-morpholinopyridin-4-yl)amino)-3,4-dihydro-2,6-naphthyridin-2(1H)-yl)-2,3-dihydro-1H-pyrido[2,3-b][1,4]oxazine-1-carboxylate: To a stirred solution of N-[2-(morpholin-4-yl)pyridin-4-yl]-5,6,7,8-tetrahy dro-2,6-naphthyridin-3-amine (240.00 mg; 0.76 mmol) and tert-butyl 7-bromo-8-methyl-1H,2H,3H-pyrido[2,3-b][1,4]oxazine-1-carboxylate (315.00 mg; 0.92 mmol) in 1,4-dioxane (3.00 mL) were added cesium carbonate (787.00 mg; 2.29 mmol) and Pd-PEPPSI-IPentCl 2-methylpyridine (o-picoline) (68.00 mg; 0.08 mmol) at room temperature under N₂ atmosphere. The resulting mixture was stirred for overnight at 100 degrees C. under N₂ atmosphere. The mixture was allowed to cool down to room temperature. The resulting mixture was poured into water, the mixture was extracted with EtOAc (50 mL×3). The combined organic layers were washed with brine dried over anhydrous Na₂SO₄ and concentrated under reduced pressure. The residue was purified by reverse flash chromatography with the following conditions: column, C18 silica gel; mobile phase, ACN in water (0.05% TFA), 65% to 75% gradient in 30 min.; detector, UV 254 nm, to afford tert-butyl 8-methyl-7-(7-{[2-(morpholin-4-yl) pyridin-4-yl]amino}-1,2,3,4-tetrahydro-2,6-naphthyridin-2-yl)-1H,2H,3H-pyrido[2,3-b][1,4]oxazine-1-carboxylate (170.00 mg; 39.5%) as a light yellow solid.

6-(8-methyl-2,3-dihydro-1H-pyrido[2,3-b][1,4]oxazin-7-yl)-N-(2-morpholinopyridin-4-yl)-5,6,7,8-tetrahydro-2,6-naphthyridin-3-amine: To a stirred solution of tert-butyl 8-methyl-7-(7-{[2-(morpholin-4-yl) pyridin-4-yl]amino}-1,2,3,4-tetrahydro-2,6-naphthyridin-2-yl)-1H,2H,3H-pyrido[2,3-b][1,4]oxazine-1-carboxylate (160.00 mg; 0.28 mmol) in dichloromethane (3.00 mL) was added TFA (1.00 mL) dropwise at room temperature under N₂ atmosphere. The resulting mixture was stirred for 1 h at room temperature under N₂ atmosphere. The resulting mixture was concentrated under reduced pressure. The crude product was purified by Prep-HPLC with the following conditions (Column: XBridge Shield RP18 OBD Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 25% to 55% in 9 min.; Wave Length: 254 nm; RT1 (min.): 7) to afford 6-{8-methyl-1H,2H,3H-pyrido[2,3-b][1,4]oxazin-7-yl}-N-[2-(morpholin-4-yl)pyridin-4-yl]-5,6,7,8-tetrahydro-2,6-naphthyridin-3-amine (45.10 mg; 34.4%) as a white solid.

HPLC: 99.6% purity, RT=3.14 min. MS: m/z=460.2 [M+H]⁺. ¹H NMR (300 MHz, DMSO-d6) 9.18 (s, 1H), 8.09 (s, 1H), 7.88 (d, J=5.7 Hz, 1H), 7.27 (s, 1H), 7.16 (d, J=1.8 Hz, 1H), 6.92 (dd, J=5.7, 1.7 Hz, 1H), 6.67 (s, 1H), 5.52 (d, J=2.8 Hz, 1H), 4.19 (t, J=4.1 Hz, 2H), 4.01 (s, 2H), 3.71 (t, J=4.8 Hz, 4H), 3.30 (d, J=4.0 Hz, 6H), 3.10 (t, J=5.7 Hz, 2H), 2.85 (t, J=5.7 Hz, 2H), 2.03 (s, 3H).

### Example 55: Synthesis of Compound 55 7-(8-methyl-2,3-dihydro-1H-pyrido[2,3-b][1,4]oxazin-7-yl)-N-(4-(4-methylpiperazin-1-yl)phenyl)-5,6,7,8-tetrahydropyrido[3,4-d]pyrimidin-2-amine

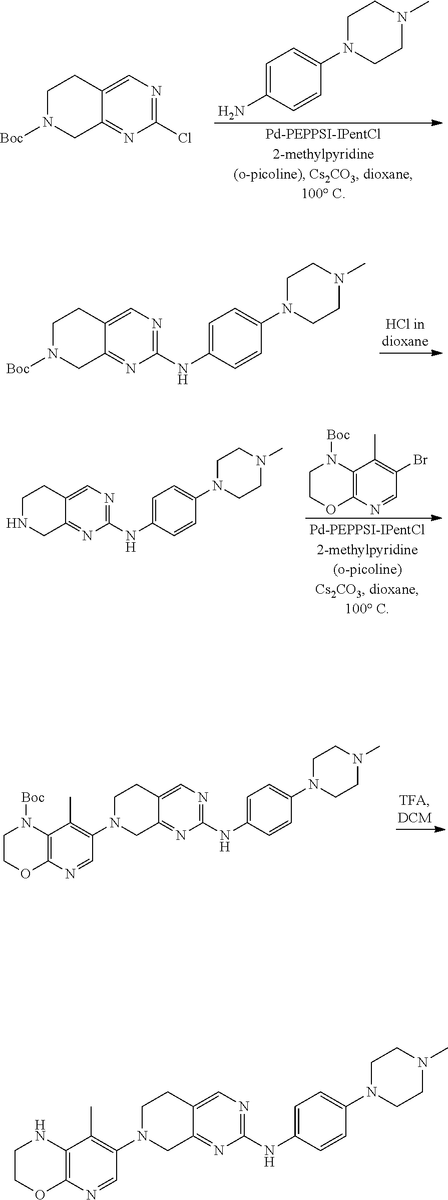

tert-butyl 2-((4-(4-methylpiperazin-1-yl)phenyl)amino)-5,8-dihydropyrido[3,4-d]pyrimidine-7(6H)-carboxylate: To a stirred mixture of tert-butyl 2-chloro-5H,6H,7H,8H-pyrido[3,4-d]pyrimidine-7-carboxylate (300.00 mg; 0.72 mmol) and 4-(4-methylpiperazin-1-yl)aniline (144.42 mg; 0.72 mmol) in 1,4-dioxane (10.00 mL) were added Pd-PEPPSI-IPentCl 2-methylpyridine (o-picoline) (64.03 mg; 0.07 mmol) and Cs₂CO₃ (496.66 mg; 1.45 mmol) at room temperature. The mixture was stirred for 2 h at 100° C. under argon atmosphere. The resulting mixture was concentrated under reduced pressure and purified by silica gel column chromatography and eluted with CH₂Cl₂/MeOH (10:1) to afford tert-butyl 2-{[4-(4-methylpiperazin-1-yl)phenyl]amino}-5H,6H,7H,8H-pyrido[3,4-d]pyrimidine-7-carboxylate (300.00 mg; 94.1%) as a light yellow oil.

N-(4-(4-methylpiperazin-1-yl)phenyl)-5,6,7,8-tetrahydropyrido[3,4-d]pyrimidin-2-amine: To a stirred solution of tert-butyl 2-{[4-(4-methylpiperazin-1-yl)phenyl]amino}-5H,6H,7H,8H-pyrido[3,4-d]pyrimidine-7-carboxylate (300.00 mg; 0.71 mmol) in DCM (10.00 mL) was added TFA (2.00 mL). The resulting mixture was stirred for 2 h at room temperature. The mixture was basified to pH=9 with sat. NaHCO₃ (aq.). The resulting mixture was quenched by ice/water and extracted with EtOAc. The combined organic layers were washed with brine and dried over anhydrous Na₂SO₄. After filtration, the filtrate was concentrated under reduced pressure. This resulted in N-[4-(4-methylpiperazin-1-yl) phenyl]-5H,6H,7H,8H-pyrido[3,4-d]pyrimidin-2-amine (180.00 mg; crude product) as a yellow oil.

tert-butyl 8-methyl-7-(2-((4-(4-methylpiperazin-1-yl)phenyl)amino)-5,8-dihydropyrido[3,4-d]pyrimidin-7(6H)-yl)-2,3-dihydro-1H-pyrido[2,3-b][1,4]oxazine-1-carboxylate: To a stirred mixture of N-[4-(4-methylpiperazin-1-yl)phenyl]-5H,6H,7H,8H-pyrido[3,4-d]pyrimidin-2-amine (180.00 mg; 0.48 mmol) and tert-butyl 7-bromo-8-methyl-1H,2H,3H-pyrido[2,3-b][1,4]oxazine-1-carboxylate (158.17 mg; 0.48 mmol) in 1,4-dioxane (10.00 mL) were added Cs₂CO₃ (329.58 mg; 0.96 mmol) and Pd-PEPPSI-IPentCl 2-methylpyridine (o-picoline) (42.49 mg; 0.05 mmol) at room temperature. The mixture was stirred for 2 h at 100° C. under argon atmosphere. The resulting mixture was concentrated under reduced pressure and purified by silica gel column chromatography and eluted with CH₂Cl₂/MeOH (1:1) to afford tert-butyl 8-methyl-7-(2-{[4-(4-methylpiperazin-1-yl)phenyl]amino}-5H,6H,7H,8H-pyrido[3,4-d]pyrimidin-7-yl)-1H,2H,3H-pyrido[2,3-b][1,4]oxazine-1-carboxylate (90.00 mg; 28.6%) as alight yellow oil.

7-(8-methyl-2,3-dihydro-1H-pyrido[2,3-b][1,4]oxazin-7-yl)-N-(4-(4-methylpiperazin-1-yl)phenyl)-5,6,7,8-tetrahydropyrido[3,4-d]pyrimidin-2-amine: To a stirred solution of tert-butyl 8-methyl-7-(2-{[4-(4-methylpiperazin-1-yl) phenyl]amino}-5H,6H,7H,8H-pyrido[3,4-d]pyrimidin-7-yl)-1H,2H,3H-pyrido[2,3-b][1,4]oxazine-1-carboxylate (80.00 mg; 0.14 mmol; 1.00 eq.) in H₂SO₄ (3.00 mL; 53.47 mmol). The resulting mixture was stirred for 0.5 h at 60° C. The resulting mixture was concentrated under reduced pressure. The residue was purified by Prep-HPLC (Column: YMC-Actus Triart C18 ExRS, 30*150 mm, 5 μm; Mobile Phase A: water (10 mmol/L NH₄HCO₃+0.05% NH₄OH), Mobile Phase B: ACN; Flow rate: 60 mL/min.; Gradient: 30% B to 60% B in 8 min., 60% B; Wave Length: 254 nm; RT1 (min.): 7) to afford 7-{8-methyl-1H,2H,3H-pyrido[2,3-b][1,4]oxazin-7-yl}-N-[4-(4-methylpiperazin-1-yl)phenyl]-5H,6H,7H,8H-pyrido[3,4-d]pyrimidin-2-amine (7.30 mg; 10.4%) as a white solid.

HPLC: 93.6% purity, RT=7.71 min. MS: m/z=473.1 [M+H]⁺. ¹H NMR (300 MHz, Methanol-d4): 8.21 (s, 1H), 7.54 (s, 2H), 7.29 (s, 1H), 6.98 (d, J=8.6 Hz, 2H), 4.64 (s, 2H), 4.33 (d, J=5.8 Hz, 2H), 3.98 (s, 2H), 3.48 (m, 2H), 3.24 (m, 4H), 2.85 (s, 2H), 2.73 (d, J=6.3 Hz, 4H), 2.42 (s, 3H), 2.16 (s, 3H).

### Example 56: Synthesis of Compound 56 2-(dimethylamino)-N-(5-((7-(8-methyl-2,3-dihydro-1H-pyrido[2,3-b][1,4]oxazin-7-yl)-5,6,7,8-tetrahydropyrido[3,4-d]pyrimidin-2-yl)amino)pyridin-2-yl)acetamide

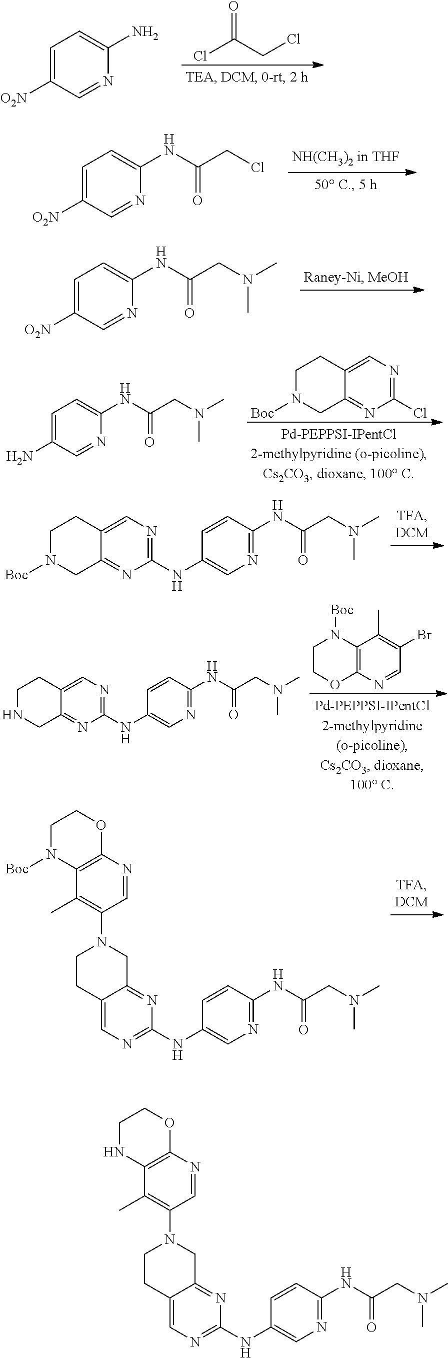

2-chloro-N-(5-nitropyridin-2-yl) acetamide: To a stirred solution of 5-nitropyridin-2-amine (2.00 g; 13.66 mmol) and 2-chloroacetyl chloride (2.44 g; 20.49 mmol) in THF (10.00 mL) was added TEA (2.91 g; 27.32 mmol). The mixture was stirred for 2 h at room temperature. The resulting mixture was quenched by ice/water and extracted with EtOAc. The combined organic layers were washed with brine and dried over anhydrous Na₂SO₄. After filtration, the filtrate was concentrated under reduced pressure. The residue was purified by silica gel column chromatography and eluted with PE/EtOAc (2:1) to afford 2-chloro-N-(5-nitropyridin-2-yl) acetamide (1.45 g; 5.43 mmol; 39.75%) as an orange brown solid.

2-(dimethylamino)-N-(5-nitropyridin-2-yl) acetamide: To a stirred solution of 2-chloro-N-(5-nitropyridin-2-yl) acetamide (1.35 g; 5.06 mmol) in dimethylamine (20.00 mL). The resulting mixture was stirred for 16 h at 50° C. under nitrogen atmosphere. The resulting mixture was concentrated under reduced pressure. The residue was purified by silica gel column chromatography and eluted with CH₂Cl₂/MeOH (5:1) to afford 2-(dimethylamino)-N-(5-nitropyridin-2-yl)acetamide (1.02 g; 75.45%) as a pink solid.

N-(5-aminopyridin-2-yl)-2-(dimethylamino) acetamide: To a solution of 2-(dimethylamino)-N-(5-nitropyridin-2-yl) acetamide (1.38 g; 5.16 mmol) in MeOH (20.00 mL) was added Raney-Ni (0.54 g; 5.93 mmol) at room temperature. After stirring for 1 h at room temperature under hydrogen atmosphere, the resulting mixture was filtered. The filter cake was washed with MeOH and then filtrate was concentrated under reduced pressure to afford N-(5-aminopyridin-2-yl)-2-(dimethylamino) acetamide (852.00 mg; crude Product) as a black solid.

tert-butyl 2-((6-(2-(dimethylamino)acetamido)pyridin-3-yl)amino)-5,8-dihydropyrido[3,4-d]pyrimidine-7(6H)-carboxylate: To a stirred solution of tert-butyl 2-chloro-5H,6H,7H,8H-pyrido[3,4-d]pyrimidine-7-carboxylate (300.00 mg; 0.96 mmol) and N-(5-aminopyridin-2-yl)-2-(dimethylamino)acetamide (187.79 mg; 0.96 mmol) in 1,4-dioxane (15.00 mL) was added Pd-PEPPSI-IPentCl 2-methylpyridine (o-picoline) (85.18 mg; 0.10 mmol) and Cs₂CO₃ (660.69 mg; 1.93 mmol). The resulting mixture was stirred for 3 h at 80° C. under nitrogen atmosphere. The resulting mixture was concentrated under reduced pressure. The residue was purified by silica gel column chromatography and eluted with CH₂Cl₂/MeOH (3:1) to afford tert-butyl 2-({6-[2-(dimethylamino) acetamido]pyridin-3-yl}amino)-5H,6H,7H,8H-pyrido[3,4-d]pyrimidine-7-carboxylate (398.00 mg; 90.95%) as a yellow oil.

2-(dimethylamino)-N-(5-((5,6,7,8-tetrahydropyrido[3,4-d]pyrimidin-2-yl)amino)pyridin-2-yl)acetamide: To a stirred solution of tert-butyl 2-({6-[2-(dimethylamino)acetamido]pyridin-3-yl}amino)-5H,6H,7H,8H-pyrido[3,4-d]pyrimidine-7-carboxylate (398.00 mg; 0.88 mmol) in DCM (10.00 mL) was added TFA (2.00 mL) in portions at room temperature. After stirring for 1 h at room temperature, the resulting mixture was concentrated under reduced pressure to afford 2-(dimethylamino)-N-[5-({5H,6H,7H,8H-pyrido[3,4-d]pyrimidin-2-yl}amino) pyridin-2-yl]acetamide (270.00 mg; crude product) as a black solid.

tert-butyl 7-(2-((6-(2-(dimethylamino)acetamido)pyridin-3-yl)amino)-5,8-dihydropyrido[3,4-d]pyrimidin-7(6H)-yl)-8-methyl-2,3-dihydro-1H-pyrido[2,3-b][1,4]oxazine-1-carboxylate: To a stirred solution of 2-(dimethylamino)-N-[5-({5H,6H,7H,8H-pyrido[3,4-d]pyrimidin-2-yl}amino)pyridin-2-yl]acetamide (179.98 mg; 0.52 mmol) and tert-butyl 7-bromo-8-methyl-1H,2H,3H-pyrido[2,3-b][1,4]oxazine-1-carboxylate (180.00 mg; 0.52 mmol) in 1,4-dioxane (10.00 mL) was added Pd-PEPPSI-IPentCl 2-methylpyridine (o-picoline) (45.98 mg; 0.05 mmol) and Cs₂CO₃ (356.65 mg; 1.04 mmol). The resulting mixture was stirred for 3 h at 100 degrees C. under nitrogen atmosphere. The resulting mixture was concentrated under reduced pressure. The residue was purified by silica gel column chromatography and eluted with CH₂Cl₂/MeOH (10:1) to afford tert-butyl 7-[2-({6-[2-(dimethylamino) acetamido]pyridin-3-yl}amino)-5H,6H,7H,8H-pyrido[3,4-d]pyrimidin-7-yl]-8-methyl-1H,2H,3H-pyrido[2,3-b][1,4]oxazine-1-carboxylate (196.00 mg; 53.73%) as a yellow solid.

2-(dimethylamino)-N-(5-((7-(8-methyl-2,3-dihydro-1H-pyrido[2,3-b][1,4]oxazin-7-yl)-5,6,7,8-tetrahydropyrido[3,4-d]pyrimidin-2-yl)amino)pyridin-2-yl)acetamide: To a stirred solution of tert-butyl 7-[2-({6-[2-(dimethylamino)acetamido]pyridin-3-yl}amino)-5H,6H,7H,8H-pyrido[3,4-d]pyrimidin-7-yl]-8-methyl-1H,2H,3H-pyrido[2,3-b][1,4]oxazine-1-carboxylate (186.00 mg; 0.27 mmol) in DCM (5.00 mL) was added TFA (1.00 mL) in portions at room temperature. After stirring for 1 h at room temperature, the resulting mixture was concentrated under reduced pressure. The crude product was purified by Prep-HPLC with the following conditions: Column: XBridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 20% B to 47% B in 9 min., 47% B; Wave Length: 254 nm; RT1 (min.): 7; Number Of Runs: 4, to afford 2-(dimethylamino)-N-{5-[(7-{8-methyl-1H,2H,3H-pyrido[2,3-b][1,4]oxazin-7-yl}-5H,6H,7H,8H-pyrido[3,4-d]pyrimidin-2-yl)amino]pyridin-2-yl}acetamide (46.20 mg; 35.02%) as a white solid.

HPLC: 95.5% purity, RT=2.74 min. MS: m/z=476.1 [M+H]⁺. ¹H NMR (300 MHz, DMSO-d6): 9.59 (s, 1H), 9.52 (s, 1H), 8.50 (d, J=2.6 Hz, 1H), 8.22 (s, 1H), 8.06 (dd, J=9.0, 2.7 Hz, 1H), 7.88 (d, J=9.0 Hz, 1H), 7.16 (s, 1H), 5.41 (s, 1H), 4.07 (t, J=4.2 Hz, 2H), 3.85 (s, 2H), 3.35 (s, 2H), 3.07 (t, J=4.2 Hz, 4H), 2.99 (d, J=7.3 Hz, 2H), 2.68 (d, J=5.8 Hz, 6H), 2.08 (s, 3H).

### Example 57: Synthesis of Compound 57 N-[4-(methanesulfonylmethyl)phenyl]-6-{8-methyl-1H,2H,3H-pyrido[2,3-b][1,4]oxazin-7-yl}-5,6,7,8-tetrahydro-2,6-naphthyridin-3-amine

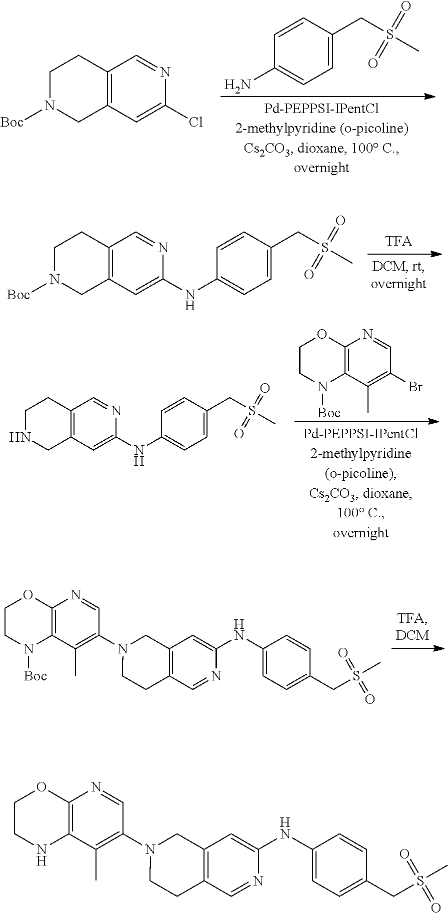

tert-butyl 7-{[4-(methanesulfonylmethyl)phenyl]amino}-1,2,3,4-tetrahydro-2,6-naphthyridine-2-carboxylate: To a solution of tert-butyl 7-chloro-1,2,3,4-tetrahydro-2,6-naphthyridine-2-carboxylate (400.00 mg; 1.49 mmol) and 4-(methanesulfonylmethyl)aniline (291.00 mg; 1.49 mmol) in 1,4-dioxane (20.00 mL) were added Cs₂CO₃ (1021.00 mg; 2.98 mmol) and Pd-PEPPSI-IPentCl 2-methylpyridine (o-picoline) (132.00 mg; 0.15 mmol). The resulting mixture was stirred for overnight at 100° C. under nitrogen atmosphere. The resulting mixture was concentrated under vacuum. The residue was purified by silica gel column chromatography and eluted with DCM/MeOH (19:1) to afford tert-butyl 7-{[4-(methanesulfonylmethyl)phenyl]amino}-1,2,3,4-tetrahydro-2,6-naphthyridine-2-carboxylate (560.00 mg; 85.2%) as a yellow solid.

N-[4-(methanesulfonylmethyl)phenyl]-5,6,7,8-tetrahydro-2,6-naphthyridin-3-amine: To a stirred solution of tert-butyl 7-{[4-(methanesulfonylmethyl)phenyl]amino}-1,2,3,4-tetrahydro-2,6-naphthyridine-2-carboxylate (550.00 mg; 1.25 mmol) in DCM (10.00 mL) was added TFA (2.00 mL) in portions at room temperature. The mixture was basified to pH [7] with saturated NaHCO₃ (aq.). The resulting mixture was extracted with DCM. The combined organic layers were washed with brine and dried over anhydrous Na₂SO₄. After filtration, the filtrate was concentrated under reduced pressure to afford N-[4-(methanesulfonylmethyl)phenyl]-5,6,7,8-tetrahydro-2,6-naphthyridin-3-amine (370.00 mg; 92.1%) as a yellow solid.

7-(7-{[4-(methanesulfonylmethyl)phenyl]amino}-1,2,3,4-tetrahydro-2,6-naphthyridin-2-yl)-8-methyl-1H,2H,3H-pyrido[2,3-b][1,4]oxazine-1-carboxylate: To a solution of N-[4-(methanesulfonylmethyl)phenyl]-5,6,7,8-tetrahydro-2,6-naphthyridin-3-amine (250.00 mg; 0.78 mmol) and tert-butyl 7-bromo-8-methyl-1H,2H,3H-pyrido[2,3-b][1,4]oxazine-1-carboxylate (259.00 mg; 0.78 mmol) in 1,4-dioxane (2.00 mL) were added Pd-PEPPSI-IPentCl 2-methylpyridine (o-picoline) (69.00 mg; 0.08 mmol) and Cs₂CO₃ (511.00 mg; 1.55 mmol). The resulting mixture was stirred for overnight at 100° C. under nitrogen atmosphere. The resulting mixture was concentrated under reduced pressure. The residue was purified by silica gel column chromatography and eluted with DCM/MeOH (6:1) to afford tert-butyl 7-(7-{[4-(methanesulfonylmethyl)phenyl]amino}-1,2,3,4-tetrahydro-2,6-naphthyridin-2-yl)-8-methyl-1H,2H,3H-pyrido[2,3-b][1,4]oxazine-1-carboxylate (290.00 mg; 59.0%) as a yellow solid.

N-[4-(methanesulfonylmethyl)phenyl]-6-{8-methyl-1H,2H,3H-pyrido[2,3-b][1,4]oxazin-7-yl}-5,6,7,8-tetrahydro-2,6-naphthyridin-3-amine: To a stirred solution of tert-butyl 7-(7-{[4-(methanesulfonylmethyl)phenyl]amino}-1,2,3,4-tetrahydro-2,6-naphthyridin-2-yl)-8-methyl-1H,2H,3H-pyrido[2,3-b][1,4]oxazine-1-carboxylate (280.00 mg; 0.44 mmol) in DCM (10.00 mL) was added TFA (2.50 mL) in portions at room temperature. The mixture was basified to pH [7] with saturated NaHCO₃ (aq.). The resulting mixture was extracted with DCM. The combined organic layers were washed with brine and dried over anhydrous Na₂SO₄. After filtration, the filtrate was concentrated under reduced pressure. The residue was purified by Prep-HPLC with the following conditions (Column: XBridge Shield RP18 OBD Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 18% to 48% in 9 min.; Wave Length: 254 nm; RT1 (min.): 7) to afford N-[4-(methanesulfonylmethyl)phenyl]-6-{8-methyl-1H,2H,3H-pyrido[2,3-b][1,4]oxazin-7-yl}-5,6,7,8-tetrahydro-2,6-naphthyridin-3-amine (66.20 mg; 32.1%) as a white solid.

HPLC: 99.7% purity, RT=3.96 min. MS: m/z=466.00[M+H]⁺. ¹H NMR (300 MHz, DMSO-d6) 8.98 (s, 1H), 8.01 (s, 1H), 7.63 (d, J=8.5 Hz, 2H), 7.26 (d, J=7.9 Hz, 3H), 6.62 (s, 1H), 5.51 (s, 1H), 4.35 (s, 2H), 4.18 (t, J=4.3 Hz, 2H), 3.99 (s, 2H), 3.30 (s, 2H), 3.08 (d, J=5.9 Hz, 2H), 2.85 (d, J=11.3 Hz, 5H), 2.03 (s, 3H).

### Example 58: Synthesis of Compound 58 6-(methanesulfonylmethyl)-N-(7-{8-methyl-1H,2H,3H-pyrido[2,3-b][1,4]oxazin-7-yl}-5H,6H,7H,8H-pyrido[3,4-d]pyrimidin-2-yl)pyridin-3-amine

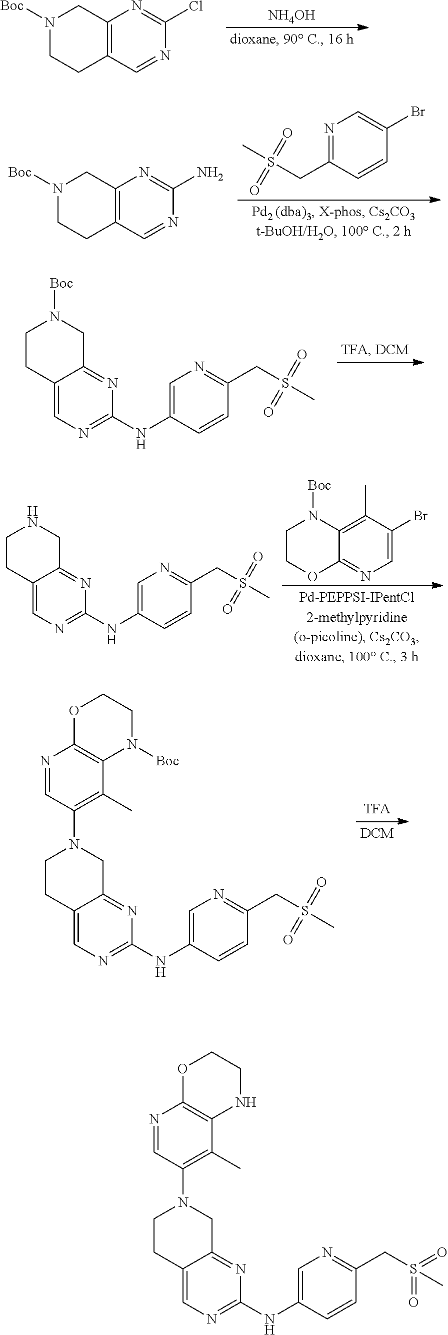

tert-butyl 2-amino-5H,6H,7H,8H-pyrido[3,4-d]pyrimidine-7-carboxylate: To a stirred solution of tert-butyl 2-chloro-5H,6H,7H,8H-pyrido[3,4-d]pyrimidine-7-carboxylate (2 g; 7.41 mmol) in 1,4-dioxane (10.00 mL) was added NH₄OH (10 mL). The resulting mixture was stirred for 16 h at 90° C. using a sealed tube. The resulting mixture was concentrated under vacuum. The residue was purified by silica gel column chromatography and eluted with CH₂Cl₂/MeOH (1:1) to afford tert-butyl 2-amino-5H,6H,7H,8H-pyrido[3,4-d]pyrimidine-7-carboxylate (600 mg; 31.84%) as yellow solid.

2-{[6-(methanesulfonylmethyl)pyridin-3-yl]amino}-5H,6H,7H,8H-pyrido[3,4-d]pyrimidine-7-carboxylate: To a solution of tert-butyl 2-amino-5H,6H,7H,8H-pyrido[3,4-d]pyrimidine-7-carboxylate (300.00 mg; 1.18 mmol) and 5-bromo-2-(methanesulfonylmethyl)pyridine (598.65 mg; 2.35 mmol) in water (1.00 mL) and t-BuOH (14.00 mL) were added Cs₂CO₃ (1210.77 mg; 3.53 mmol), Pd₂(dba)₃ (119.73 mg; 0.12 mmol) and Xphos (118.10 mg; 0.24 mmol). The resulting mixture was stirred for 3 h at 100° C. under nitrogen atmosphere. The resulting mixture was concentrated under reduced pressure. The residue was purified by silica gel column chromatography and eluted with DCM/MEOH (9:1) to afford tert-butyl 2-{[6-(methanesulfonylmethyl)pyridin-3-yl]amino}-5H,6H,7H,8H-pyrido[3,4-d]pyrimidine-7-carboxylate (510.00 mg; 88.01%) as a yellow solid.

6-(methanesulfonylmethyl)-N-{5H,6H,7H,8H-pyrido[3,4-d]pyrimidin-2-yl}pyridin-3-amine: To a stirred mixture of tert-butyl 2-{[6 (methanesulfonylmethyl) pyridin-3-yl]amino}-5H,6H,7H,8H-pyrido[3,4-d]pyrimidine-7-carboxylate (500.00 mg; 1.02 mmol) in DCM (10.00 mL) and was added TFA (2.00 mL) at room temperature. After stirring for 1 h at room temperature, the resulting mixture was extracted with CH₂Cl₂ (20 mL*3) under a basic environment. The combined organic layers were washed with brine (20 mL) and dried over anhydrous Na₂SO₄. After filtration, the filtrate was concentrated under reduced pressure. The resulting mixture was filtered and the filter cake was washed with CH₂Cl₂. The filtrate was concentrated under reduced pressure to afford 6-(methanesulfonylmethyl)-N-{5H,6H,7H,8H-pyrido[3,4-d]pyrimidin-2-yl}pyridin-3-amine (210.00 mg; crude product) as a yellow solid.

tert-butyl 7-(2-{[6-(methanesulfonylmethyl)pyridin-3-yl]amino}-5H,6H,7H,8H-pyrido[3,4-d]pyrimidin-7-yl)-8-methyl-1H,2H,3H-pyrido[2,3-b][1,4]oxazine-1-carboxylate: To a solution of 6-N-{5H,6H,7H,8H-pyrido[3,4-d]pyrimidin-2-yl}pyridin-3-amine (200.00 mg; 0.62 mmol) and tert-butyl 7-bromo-8-methyl-1H,2H,3H-pyrido[2,3-b][1,4]oxazine-1-carboxylate (309.31 mg; 0.93 mmol) in 1,4-dioxane (15.00 mL) were added Cs₂CO₃ (637.22 mg; 1.86 mmol) and Pd-PEPPSI-IPentCl 2-methylpyridine (o-picoline) (54.77 mg; 0.06 mmol). The resulting mixture was stirred for 3 h at 100° C. under nitrogen atmosphere. The mixture was concentrated under reduced pressure. The residue was purified by silica gel column chromatography and eluted with CH₂Cl₂/MeOH (10:1) to afford tert-butyl 7-(2-{[6-(methanesulfonylmethyl)pyridin-3-yl]amino}-5H,6H,7H,8H-pyrido[3,4-d]pyrimidin-7-yl)-8-methyl-1H,2H,3H-pyrido[2,3-b][1,4]oxazine-1-carboxylate (196.00 mg; 49.36%) as a yellow solid.

6-(methanesulfonylmethyl)-N-(7-{8-methyl-1H,2H,3H-pyrido[2,3-b][1,4]oxazin-7-yl}-5H,6H,7H,8H-pyrido[3,4-d]pyrimidin-2-yl)pyridin-3-amine: To a stirred mixture of tert-butyl 7-(2-{[6-(methanesulfonylmethyl) pyridin-3-yl]amino}-5H,6H,7H,8H-pyrido[3,4-d]pyrimidin-7-yl)-8-methyl-1H,2H,3H-pyrido[2,3-b][1,4]oxazine-1-carboxylate (190.00 mg; 0.30 mmol) in DCM (6.00 mL) and was added TFA (1.00 mL) at room temperature. After stirring for 1 h at room temperature, the mixture was concentrated under reduced pressure. The crude product was purified by Prep-HPLC (Column: XBridge Shield RP18 OBD Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 20% B to 50% B in 9 min., 50% B; Wave Length: 254 nm; RT1 (min.): 7) to afford 6-(methanesulfonylmethyl)-N-(7-{8-methyl-1H,2H,3H-pyrido[2,3-b][1,4]oxazin-7-yl}-5H,6H,7H,8H-pyrido[3,4-d]pyrimidin-2-yl)pyridin-3-amine (34.10 mg; 24.06%) as a white solid.

HPLC: 97.6% purity, RT=3.08 min. MS: m/z=468.20[M+H]⁺. ¹H NMR (300 MHz, DMSO-d6): 9.87 (s, 1H), 8.89 (d, J=2.6 Hz, 1H), 8.39 (s, 1H), 8.27 (dd, J=8.5, 2.7 Hz, 1H), 7.40 (d, J=8.5 Hz, 1H), 7.29 (s, 1H), 5.54 (s, 1H), 4.53 (s, 2H), 4.19 (t, J=4.5 Hz, 2H), 4.00 (s, 2H), 3.30 (s, 2H), 3.13 (s, 2H), 2.98 (s, 3H), 2.82 (s, 2H), 2.06 (d, J=10.9 Hz, 3H).

### Example 59: Synthesis of Compound 59 N-{4-[(dimethylamino)methyl]phenyl}-7-{8-methyl-1H,2H,3H-pyrido[2,3-b][1,4]oxazin-7-yl}-5H,6H,7H,8H-pyrido[3,4-d]pyrimidin-2-amine; formic acid

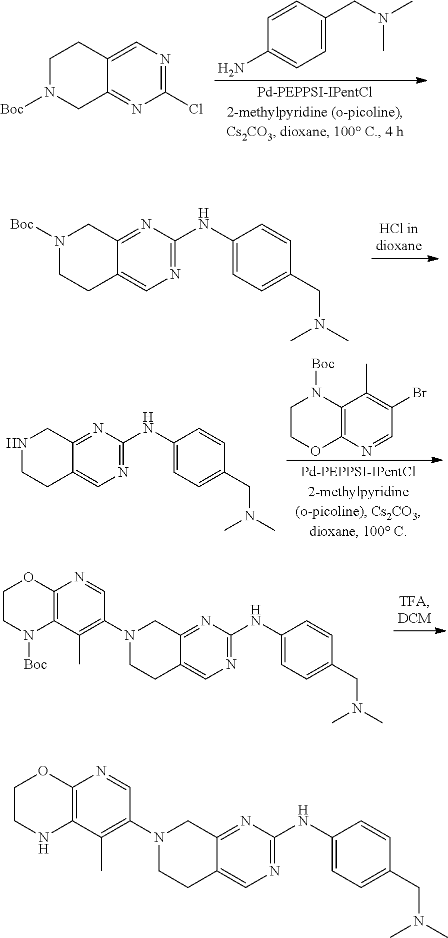

tert-butyl 2-({4-[(dimethylamino)methyl]phenyl}amino)-5H,6H,7H,8H-pyrido[3,4-d]pyrimidine-7-carboxylate: To a stirred solution of tert-butyl 2-chloro-5H,6H,7H,8H-pyrido[3,4-d]pyrimidine-7-carboxylate (300.00 mg; 1.11 mmol) and 4-[(dimethylamino)methyl]aniline (211.05 mg; 1.33 mmol) in 1,4-dioxane (5.00 mL) were added Pd-PEPPSI-IPentCl 2-methylpyridine (o-picoline) (49.18 mg; 0.06 mmol) and cesium carbonate (762.92 mg; 2.22 mmol) at room temperature. The resulting mixture was stirred for 4 h at 100° C. under N₂ atmosphere. The residue was purified by silica gel column and eluted with 30% EtOAc in PE to afford tert-butyl 2-({4-[(dimethylamino)methyl]phenyl}amino)-5H,6H,7H,8H-pyrido[3,4-d]pyrimidine-7-carboxylate (400.00 mg; 93.8%) as a light yellow solid.

N-{4-[(dimethylamino)methyl]phenyl}-5H,6H,7H,8H-pyrido[3,4-d]pyrimidin-2-amine: To a stirred solution of tert-butyl 2-({4-[(dimethylamino)methyl]phenyl}amino)-5H,6H,7H,8H-pyrido[3,4-d]pyrimidine-7-carboxylate (380.00 mg; 0.99 mmol) in MeOH (2.00 mL) was added HCl (4 N in dioxane) (3.00 mL) at room temperature under N₂ atmosphere. The resulting mixture was stirred for 1 h at room temperature under N₂ atmosphere. The resulting mixture was diluted with water (15 mL), extracted with EtOAc (3×10 mL). The combined organic layers were washed with brine (15 mL) and dried over anhydrous Na₂SO₄. The resulting mixture was concentrated under reduced pressure to afford N-{4-[(dimethylamino)methyl]phenyl}-5H,6H,7H,8H-pyrido[3,4-d]pyrimidin-2-amine (350.00 mg; crude product) as a brown yellow solid.

tert-butyl 7-[2-({4-[(dimethylamino)methyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate: To a stirred solution of N-{4-[(dimethylamino)methyl]phenyl}-5H,6H,7H,8H-pyrido[3,4-d]pyrimidin-2-amine (300.00 mg; 1.06 mmol) and tert-butyl 7-bromo-8-methyl-1H,2H,3H-pyrido[2,3-b][1,4]oxazine-1-carboxylate (622.98 mg; 1.27 mmol) in 1,4-dioxane (3.00 mL) were added Pd-PEPPSI-IPentCl 2-methylpyridine (o-picoline) (93.63 mg; 0.11 mmol) and Cs₂CO₃ (726.18 mg; 2.12 mmol) at room temperature. The resulting mixture was stirred for 1 h at 100° C. under N₂ atmosphere. The residue was purified by silica gel column and eluted with 50% EtOAc in PE to afford tert-butyl 7-[2-({4-[(dimethylamino)methyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate (260.00 mg; 45.6%) as a brown yellow solid.

N-{4-[(dimethylamino)methyl]phenyl}-7-{8-methyl-1H,2H,3H-pyrido[2,3-b][1,4]oxazin-7-yl}-5H,6H,7H,8H-pyrido[3,4-d]pyrimidin-2-amine; formic acid: To a stirred solution of tert-butyl 7-[2-({4-[(dimethylamino)methyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate (150.00 mg; 0.28 mmol) in DCM (3.00 mL) was added TFA (1.00 mL) at room temperature under N₂ atmosphere. The resulting mixture was stirred for 1 h at room temperature under N₂ atmosphere. The resulting mixture was concentrated and the residue was purified by Prep-HPLC with the following condition: Column: Xselect CSH C18 OBD Column 30*150 mm, 5 μm, n; Mobile Phase A: water (0.1% FA), Mobile Phase B: ACN; Flow rate: 60 mL/min.; Gradient: 6% to 26% in 8 min.; Wave Length: 254/220 nm; RT1 (min.): 6.3; Number Of Runs: 1, to afford N-{4-[(dimethylamino)methyl]phenyl}-7-{8-methyl-1H,2H,3H-pyrido[2,3-b][1,4]oxazin-7-yl}-5H,6H,7H,8H-pyrido[3,4-d]pyrimidin-2-amine; formic acid (8.00 mg; 5.8%) as a brown solid.

HPLC: 98.0% purity, RT=2.92 min. MS: m/z=432.30 [M+H]⁺. ¹H NMR (400 MHz, Methanol-d4) 8.55 (s, 1H), 8.25 (s, 1H), 7.78 (d, J=8.1 Hz, 2H), 7.34 (d, J=8.2 Hz, 2H), 7.22 (s, 1H), 4.34-4.27 (m, 2H), 4.00 (d, J=11.3 Hz, 4H), 3.41 (t, J=4.4 Hz, 2H), 3.17 (t, J=5.7 Hz, 2H), 2.83 (t, J=5.4 Hz, 2H), 2.67 (s, 6H), 2.13 (s, 3H).

### Example 60: Synthesis of Compound 60 7-{8-methyl-1H,2H,3H-pyrido[2,3-b][1,4]oxazin-7-yl}-N-{3-[(morpholin-4-yl)methyl]phenyl}-5H,6H,7H,8H-pyrido[3,4-d]pyrimidin-2-amine

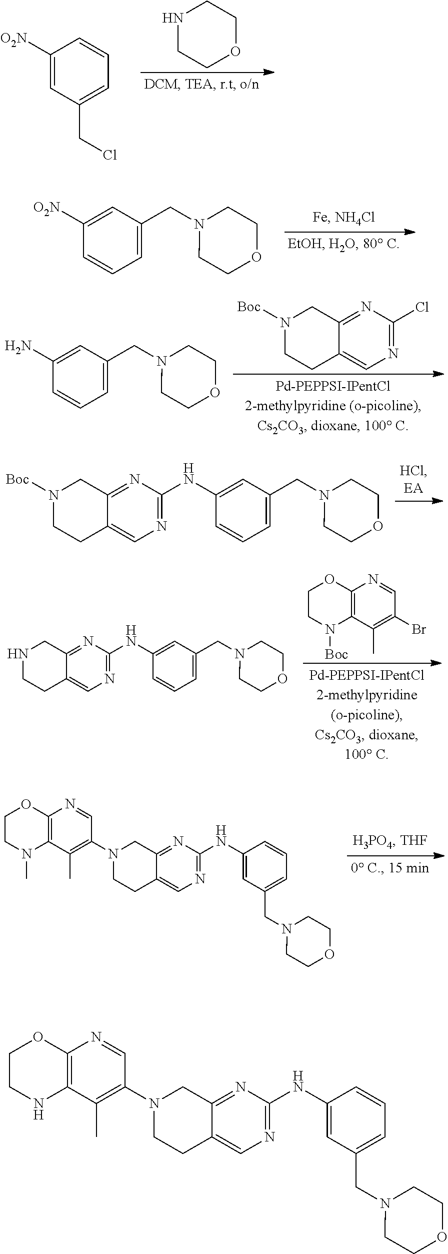

4-[(3-nitrophenyl) methyl] morpholine: To a stirred mixture of 1-(chloromethyl)-3-nitrobenzene (1.00 g; 5.54 mmol) and TEA (2.45 mL; 16.71 mmol) in DCM (15.00 mL) was added morpholine (0.77 mL; 8.40 mmol) in portions at 0° C. The resulting mixture was stirred for overnight at room temperature under nitrogen atmosphere. water was added to the reaction. The resulting mixture was extracted with EA (3×50 mL). The combined organic layers were concentrated under reduced pressure to afford 4-[(3-nitrophenyl) methyl] morpholine (1.20 g; crude product) as a yellow oil.

3-[(morpholin-4-yl) methyl]aniline: A solution of 4-[(3-nitrophenyl) methyl] morpholine (1.18 g; 5.26 mmol), NH₄Cl (1.20 g; 21.31 mmol) and Fe (1.60 g; 27.14 mmol) in EtOH (6.00 mL) and H₂O (4.00 mL) was stirred for 1 h at 80° C. under N₂ atmosphere. The resulting mixture was concentrated under reduced pressure. The residue was purified by silica gel column chromatography and eluted with 5% MeOH in DCM to afford 3-[(morpholin-4-yl) methyl]aniline (1.23 g; 99.9%) as a brown solid.

tert-butyl 2-({3-[(morpholin-4-yl)methyl]phenyl}amino)-5H,6H,7H,8H-pyrido[3,4-d]pyrimidine-7-carboxylate: To a stirred mixture of tert-butyl 2-chloro-5H,6H,7H,8H-pyrido[3,4-d]pyrimidine-7-carboxylate (300.00 mg; 1.08 mmol) and 3-[(morpholin-4-yl)methyl]aniline (330.00 mg; 1.41 mmol) in 1,4-dioxane (5.00 mL) was added Cs₂CO₃ (910.00 mg; 2.65 mmol) and Pd-PEPPSI-IPentCl 2-methylpyridine (o-picoline) (94.00 mg; 0.11 mmol) in portions at room temperature. The resulting mixture was stirred for 1 h at 100° C. under nitrogen atmosphere. The resulting mixture was concentrated under reduced pressure. The residue was purified by silica gel column chromatography and eluted with 5% MeOH in DCM to afford tert-butyl 2-({3-[(morpholin-4-yl)methyl]phenyl}amino)-5H,6H,7H,8H-pyrido[3,4-d]pyrimidine-7-carboxylate (500.00 mg; 98.8%) as a brown solid.

N-{3-[(morpholin-4-yl)methyl]phenyl}-5H,6H,7H,8H-pyrido[3,4-d]pyrimidin-2-amine: To a stirred mixture of tert-butyl 2-({3-[(morpholin-4-yl)methyl]phenyl}amino)-5H,6H,7H,8H-pyrido[3,4-d]pyrimidine-7-carboxylate (490.00 mg; 1.05 mmol) in MeOH (2.00 mL) was added hydrochloric acid 4.0 M solution in ethyl acetate (5.00 mL; 20.00 mmol) dropwise at 0° C. The resulting mixture was stirred for 1 h at room temperature under nitrogen atmosphere. Water was added to the reaction. The resulting mixture was extracted with EA (3×20 mL). The combined organic layers were dried over anhydrous Na₂SO₄. After filtration, the filtrate was concentrated under reduced pressure to afford N-{3-[(morpholin-4-yl) methyl]phenyl}-5H,6H,7H,8H-pyrido[3,4-d]pyrimidin-2-amine (350.00 mg; crude product) as a yellow solid.

tert-butyl 8-methyl-7-[2-({3-[(morpholin-4-yl)methyl]phenyl}amino)-5H,6H,7H,8H-pyrido[3,4-d]pyrimidin-7-yl]-1H,2H,3H-pyrido[2,3-b][1,4]oxazine-1-carboxylate: To a stirred mixture of N-{3-[(morpholin-4-yl)methyl]phenyl}-5H,6H,7H,8H-pyrido[3,4-d]pyrimidin-2-amine (330.00 mg; 0.96 mmol) and tert-butyl 7-bromo-8-methyl-1H,2H,3H-pyrido[2,3-b][1,4]oxazine-1-carboxylate (500.00 mg; 1.50 mmol) in 1,4-dioxane (5.00 mL) was added Cs₂CO₃ (700.00 mg; 2.04 mmol) and Pd-PEPPSI-IPentCl 2-methylpyridine (o-picoline) (100.00 mg; 0.11 mmol) in portions at room temperature. The resulting mixture was stirred for overnight at 100° C. under nitrogen atmosphere. The resulting mixture was concentrated under reduced pressure. The residue was purified by silica gel column chromatography and eluted with 5% MeOH in DCM. The residue was purified by reverse flash chromatography with the following conditions: column, C18 silica gel; mobile phase, ACN in water, 25% to 35% gradient in 10 min.; detector, UV 254 nm, to afford tert-butyl 8-methyl-7-[2-({3-[(morpholin-4-yl)methyl]phenyl}amino)-5H,6H,7H,8H-pyrido[3,4-d]pyrimidin-7-yl]-1H,2H,3H-pyrido[2,3-b][1,4]oxazine-1-carboxylate (400.00 mg; 70.3%) as a yellow solid.

7-{8-methyl-1H,2H,3H-pyrido[2,3-b][1,4]oxazin-7-yl}-N-{3-[(morpholin-4-yl)methyl]phenyl}-5H,6H,7H,8H-pyrido[3,4-d]pyrimidin-2-amine: To a stirred mixture of tert-butyl 8-methyl-7-[2-({3-[(morpholin-4-yl)methyl]phenyl}amino)-5H,6H,7H,8H-pyrido[3,4-d]pyrimidin-7-yl]-1H,2H,3H-pyrido[2,3-b][1,4]oxazine-1-carboxylate (390.00 mg; 0.66 mmol) in THF (2.00 mL) was added H₃PO₄ (6.00 mL; 109.01 mmol) dropwise at 0° C. The resulting mixture was stirred for 15 min. at room temperature under nitrogen atmosphere. The reaction was added to a solution of 250 mL ice water. The mixture was basified to pH 14 with cold NaOH (aq.). The resulting mixture was extracted with DCM (3×50 mL). The combined organic layers were dried over anhydrous Na₂SO₄. After filtration, the filtrate was concentrated under reduced pressure. The crude product was purified by Prep-HPLC with the following conditions (Column: XBridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 24% B to 54% B in 9 min., 54% B; Wave Length: 254 nm; RTT (min.): 7) to afford 7-{8-methyl-1H,2H,3H-pyrido[2,3-b][1,4]oxazin-7-yl}-N-{3-[(morpholin-4-yl)methyl]phenyl}-5H,6H,7H,8H-pyrido[3,4-d]pyrimidin-2-amine (93.00 mg; 29.7%) as a white solid.

HPLC: 99.6% purity, RT=2.89 min. MS: m/z=474.10[M+H]⁺. ¹H NMR (400 MHz, DMSO-d6) 9.46 (s, 1H), 8.32 (s, 1H), 7.75-7.59 (m, 2H), 7.29 (s, 1H), 7.19 (t, J=7.7 Hz, 1H), 6.85 (d, J=7.5 Hz, 1H), 5.52 (d, J=2.9 Hz, 1H), 4.20 (t, J=4.4 Hz, 2H), 3.96 (s, 2H), 3.57 (t, J=4.6 Hz, 4H), 3.41 (s, 2H), 3.31 (dt, J=6.7, 3.6 Hz, 2H), 3.12 (t, J=5.7 Hz, 2H), 2.79 (t, J=5.7 Hz, 2H), 2.36 (t, J=4.6 Hz, 4H), 2.05 (s, 3H).

### Example 61: Synthesis of Compound 61 7-{8-methyl-1H,2H,3H-pyrido[2,3-b][1,4]oxazin-7-yl}-N-[3-(4-methylpiperazin-1-yl)phenyl]-5H,6H,7H,8H-pyrido[3,4-d]pyrimidin-2-amine

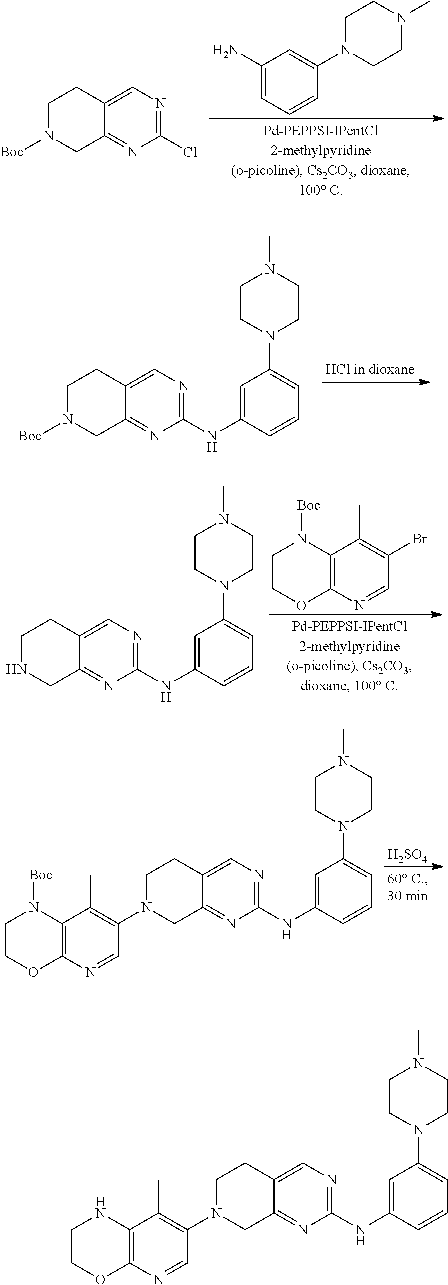

tert-butyl 2-{[3-(4-methylpiperazin-1-yl)phenyl]amino}-5H,6H,7H,8H-pyrido[3,4-d]pyrimidine-7-carboxylate: To a stirred mixture of tert-butyl 2-chloro-5H,6H,7H,8H-pyrido[3,4-d]pyrimidine-7-carboxylate (300.00 mg; 0.96 mmol) and 3-(4-methylpiperazin-1-yl)aniline (184.23 mg; 0.96 mmol) in 1,4-dioxane (15.00 mL) were added Cs₂CO₃ (660.69 mg; 1.93 mmol) and Pd-PEPPSI-IPentCl 2-methylpyridine (o-picoline) (85.18 mg; 0.10 mmol) at room temperature. The mixture was stirred for 2 h at 100° C. under argon atmosphere. The resulting mixture was concentrated under reduced pressure and purified by silica gel column chromatography and eluted with CH₂Cl₂/MeOH (10:1) to afford tert-butyl 2-{[3-(4-methylpiperazin-1-yl)phenyl]amino}-5H,6H,7H,8H-pyrido[3,4-d]pyrimidine-7-carboxylate (450.00 mg; 109.0%) as a yellow oil.

N-[3-(4-methylpiperazin-1-yl)phenyl]-5H,6H,7H,8H-pyrido[3,4-d]pyrimidin-2-amine: To a stirred solution of tert-butyl 2-{[3-(4-methylpiperazin-1-yl)phenyl]amino}-5H,6H,7H,8H-pyrido[3,4-d]pyrimidine-7-carboxylate (450.00 mg; 1.06 mmol) in DCM (10.00 mL) was added TFA (2.00 mL). The resulting mixture was stirred for 2 h at room temperature. The mixture was basified to pH=9 with sat. NaHCO₃ (aq.). The resulting mixture was quenched by ice/water and extracted with EtOAc. The combined organic layers were washed with brine and dried over anhydrous Na₂SO₄. After filtration, the filtrate was concentrated under reduced pressure. This resulted in N-[3-(4-methylpiperazin-1-yl)phenyl]-5H,6H,7H,8H-pyrido[3,4-d]pyrimidin-2-amine (260.00 mg; crude product) as a yellow solid.

tert-butyl 8-methyl-7-(2-{[3-(4-methylpiperazin-1-yl)phenyl]amino}-5H,6H,7H,8H-pyrido[3,4-d]pyrimidin-7-yl)-1H,2H,3H-pyrido[2,3-b][1,4]oxazine-1-carboxylate: To a stirred mixture of N-[3-(4-methylpiperazin-1-yl)phenyl]-5H,6H,7H,8H-pyrido[3,4-d]pyrimidin-2-amine (260.00 mg; 0.69 mmol) and tert-butyl 7-bromo-8-methyl-1H,2H,3H-pyrido[2,3-b][1,4]oxazine-1-carboxylate (228.47 mg; 0.69 mmol) in 1,4-dioxane (15.00 mL) were added Cs₂CO₃ (476.06 mg; 1.39 mmol) and Pd-PEPPSI-IPentCl 2-methylpyridine (o-picoline) (61.38 mg; 0.07 mmol) at room temperature. The mixture was stirred for 2 h at 100° C. under argon atmosphere. The resulting mixture was concentrated under reduced pressure and purified by silica gel column chromatography and eluted with DCM/MeOH (10:1) to afford tert-butyl 8-methyl-7-(2-{[3-(4-methylpiperazin-1-yl)phenyl]amino}-5H,6H,7H,8H-pyrido[3,4-d]pyrimidin-7-yl)-1H,2H,3H-pyrido[2,3-b][1,4]oxazine-1-carboxylate (150.00 mg; 37.3%) as a yellow oil.

7-{8-methyl-1H,2H,3H-pyrido[2,3-b][1,4]oxazin-7-yl}-N-[3-(4-methylpiperazin-1-yl)phenyl]-5H,6H,7H,8H-pyrido[3,4-d]pyrimidin-2-amine: To a stirred solution of tert-butyl 8-methyl-7-(2-{[3-(4-methylpiperazin-1-yl)phenyl]amino}-5H,6H,7H,8H-pyrido[3,4-d]pyrimidin-7-yl)-1H,2H,3H-pyrido[2,3-b][1,4]oxazine-1-carboxylate (140.00 mg) in H₂SO₄ (5.00 mL). The resulting mixture was stirred for 0.5 h at 60° C. The resulting mixture was concentrated under reduced pressure. The residue was purified by Prep-HPLC (Column: XBridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 22% B to 50% B in 9 min., 50% B; Wave Length: 254 nm; RTT (min.): 7) to afford 7-{8-methyl-1H,2H,3H-pyrido[2,3-b][1,4]oxazin-7-yl}-N-[3-(4-methylpiperazin-1-yl)phenyl]-5H,6H,7H,8H-pyrido[3,4-d]pyrimidin-2-amine (4.30 mg; 0.03%) as a brown semi solid.

HPLC: 95.0% purity, RT=3.00 min. MS: m/z=473.15 [M+H]⁺. ¹H NMR (300 MHz, Methanol-d4): 8.27 (s, 1H), 7.55-7.48 (m, 1H), 7.27-7.15 (m, 3H), 6.74-6.63 (m, 1H), 4.34 (dd, J=5.1, 3.7 Hz, 2H), 4.01 (s, 2H), 3.45-3.75 (d, J=8.9 Hz, 10H), 3.21 (t, J=5.7 Hz, 2H), 2.97 (s, 3H), 2.86 (t, J=5.7 Hz, 2H), 2.17 (s, 3H).

### Example 62: Synthesis of Compound 62 3-methyl-N-(7-{8-methyl-1H,2H,3H-pyrido[2,3-b][1,4]oxazin-7-yl}-5H,6H,7H,8H-pyrido[3,4-d]pyrimidin-2-yl)-2,3,4,5-tetrahydro-1H-3-benzazepin-7-amine

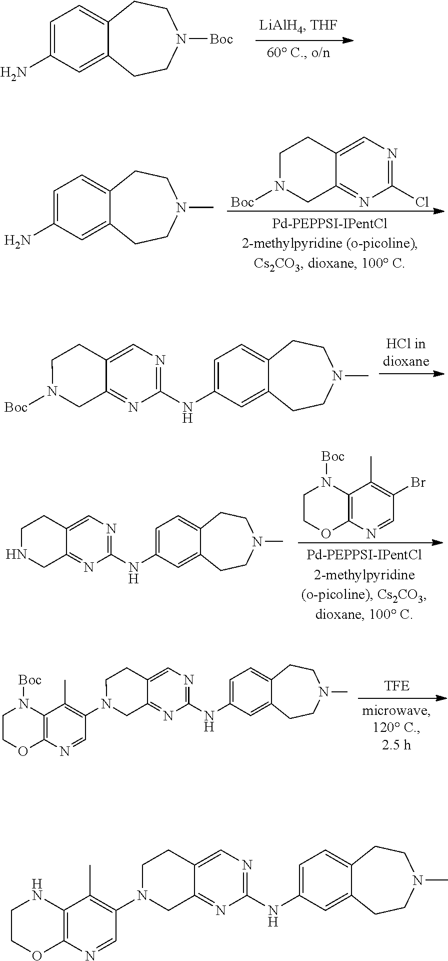

3-methyl-2,3,4,5-tetrahydro-1H-3-benzazepin-7-amine: To a stirred mixture of tert-butyl 7-amino-2,3,4,5-tetrahydro-1H-3-benzazepine-3-carboxylate (500.00 mg; 1.81 mmol) in THF (20.00 mL) was added LiAlH₄(1M in THF) (4.60 mL; 4.60 mmol) dropwise at 0° C. The resulting mixture was stirred for overnight at 60° C. under nitrogen atmosphere. The reaction was quenched with water (0.5 mL, very slow addition), 3N solution of NaOH (0.5 mL) and water (0.5 mL) at 0° C. The mixture was stirred for 5 min. at room temperature. The mixture was added water. The mixture was extracted with EA (3×80 mL). The combined organic layers were dried over anhydrous Na₂SO₄. After filtration, the filtrate was concentrated under reduced pressure to afford 3-methyl-2,3,4,5-tetrahydro-1H-3-benzazepin-7-amine (400.00 mg; crude product) as a brown oil.

tert-butyl 2-[(3-methyl-2,3,4,5-tetrahydro-1H-3-benzazepin-7-yl)amino]-5H,6H,7H,8H-pyrido[3,4-d]pyrimidine-7-carboxylate: To a stirred mixture of tert-butyl 2-chloro-5H,6H,7H,8H-pyrido[3,4-d]pyrimidine-7-carboxylate (300.00 mg; 1.08 mmol) and 3-methyl-2,3,4,5-tetrahydro-1H-3-benzazepin-7-amine (300.00 mg; 1.12 mmol) in 1,4-dioxane (4.00 mL) were added Cs₂CO₃ (725.00 mg; 2.11 mmol) and Pd-PEPPSI-IPentCl 2-methylpyridine (o-picoline) (94.00 mg; 0.11 mmol) in portions at room temperature. The resulting mixture was stirred for overnight at 90° C. under nitrogen atmosphere. Water was added to the reaction. The resulting mixture was extracted with EA (3×30 mL). The combined organic layers were washed with brine and dried over anhydrous Na₂SO₄. After filtration, the filtrate was concentrated under reduced pressure. The residue was purified by reverse flash chromatography with the following conditions: column, C18 silica gel; mobile phase, ACN in water, 55% to 65% gradient in 10 min.; detector, UV 254 nm, to afford tert-butyl 2-[(3-methyl-2,3,4,5-tetrahydro-1H-3-benzazepin-7-yl)amino]-5H,6H,7H,8H-pyrido[3,4-d]pyrimidine-7-carboxylate (470.00 mg; 100.0%) as a yellow oil.

3-methyl-N-{5H,6H,7H,8H-pyrido[3,4-d]pyrimidin-2-yl}-2,3,4,5-tetrahydro-1H-3-benzazepin-7-amine: A solution of tert-butyl 2-[(3-methyl-2,3,4,5-tetrahydro-1H-3-benzazepin-7-yl) amino]-5H,6H,7H,8H-pyrido[3,4-d]pyrimidine-7-carboxylate (450.00 mg; 1.04 mmol) in HCl (gas) in 1,4-dioxane (7.00 mL) was stirred for 1 h at room temperature under N₂ atmosphere. The resulting mixture was added water and basified to PH=10 with Na₂CO₃ (aq.). The resulting mixture was extracted with EA (3×15 mL). The combined organic layers were washed with brine and dried over anhydrous Na₂SO₄. After filtration, the filtrate was concentrated under reduced pressure to afford 3-methyl-N-{5H,6H,7H,8H-pyrido[3,4-d]pyrimidin-2-yl}-2,3,4,5-tetrahydro-1H-3-benzazepin-7-amine (200.00 mg; Crude Product) as a brown oil.

tert-butyl 8-methyl-7-{2-[(3-methyl-2,3,4,5-tetrahydro-1H-3-benzazepin-7-yl)amino]-5H,6H,7H,8H-pyrido[3,4-d]pyrimidin-7-yl}-1H,2H,3H-pyrido[2,3-b][1,4]oxazine-1-carboxylate: To a stirred mixture of 3-methyl-N-{5H,6H,7H,8H-pyrido[3,4-d]pyrimidin-2-yl}-2,3,4,5-tetrahydro-1H-3-benzazepin-7-amine (180.00 mg; 0.56 mmol) and tert-butyl 7-bromo-8-methyl-1H,2H,3H-pyrido[2,3-b][1,4]oxazine-1-carboxylate (300.00 mg; 0.90 mmol) in 1,4-dioxane (3.00 mL) were added Cs₂CO₃ (450.00 mg; 1.31 mmol) and Pd-PEPPSI-IPentCl 2-methylpyridine (o-picoline) (60.00 mg; 0.07 mmol) in portions at room temperature. The resulting mixture was stirred for 2 h at 100° C. under nitrogen atmosphere. The resulting mixture was concentrated under reduced pressure. The residue was purified by silica gel column chromatography and eluted with 90% EA in PE to afford tert-butyl 8-methyl-7-{2-[(3-methyl-2,3,4,5-tetrahydro-1H-3-benzazepin-7-yl)amino]-5H,6H,7H,8H-pyrido[3,4-d]pyrimidin-7-yl}-1H,2H,3H-pyrido[2,3-b][1,4]oxazine-1-carboxylate (110.00 mg; 34.1%) as a yellow solid.

3-methyl-N-(7-{8-methyl-1H,2H,3H-pyrido[2,3-b][1,4]oxazin-7-yl}-5H,6H,7H,8H-pyrido[3,4-d]pyrimidin-2-yl)-2,3,4,5-tetrahydro-1H-3-benzazepin-7-amine: To a stirred mixture of tert-butyl 8-methyl-7-{2-[(3-methyl-2,3,4,5-tetrahydro-1H-3-benzazepin-7-yl)amino]-5H,6H,7H,8H-pyrido[3,4-d]pyrimidin-7-yl}-1H,2H,3H-pyrido[2,3-b][1,4]oxazine-1-carboxylate (190.00 mg; 0.28 mmol) in TFE (4.00 mL) was stirred for 2.5 h at 120° C. under nitrogen atmosphere. The residue was purified by reverse flash chromatography with the following conditions: column, C18 silica gel; mobile phase, ACN in water (5 mM NH₄HCO₃), 10% to 50% gradient in 10 min.; detector, UV 254 nm. The product was purified by Prep-HPLC with the following conditions: (Column: XBridge Prep OBD C18 Column, 30*150 mm, 5 μm; Mobile Phase A: water (10 mmol/L NH₄HCO₃+0.1% NH₄OH), Mobile Phase B: ACN; Flow rate: 60 mL/min.; Gradient: 22% B to 50% B in 9 min., 50% B; Wave Length: 254 nm; RT1 (min.): 7) to afford 3-methyl-N-(7-{8-methyl-1H,2H,3H-pyrido[2,3-b][1,4]oxazin-7-yl}-5H,6H,7H,8H-pyrido[3,4-d]pyrimidin-2-yl)-2,3,4,5-tetrahydro-1H-3-benzazepin-7-amine (13.00 mg; 9.9%) as a yellow solid.

HPLC: 98.76% purity, RT=3.02 min. MS: m/z=458.10 [M+H]⁺. ¹H NMR (300 MHz, DMSO-d6) A 9.31 (s, 1H), 8.30 (s, 1H), 7.55-7.38 (m, 2H), 7.28 (s, 1H), 6.98 (d, J=8.2 Hz, 1H), 5.51 (d, J=2.6 Hz, 1H), 4.19 (t, J=4.4 Hz, 2H), 3.94 (s, 2H), 3.31 (t, J=4.0 Hz, 2H), 3.12 (t, J=5.6 Hz, 2H), 2.79 (d, J=5.7 Hz, 6H), 2.46 (d, J=7.5 Hz, 4H), 2.25 (s, 3H), 2.04 (s, 3H).

### Example 63: Synthesis of Compound 63 7-{8-chloro-1H,2H,3H-pyrido[2,3-b][1,4]oxazin-7-yl}-N-[4-(methanesulfonylmethyl)phenyl]-5H,6H,7H,8H-pyrido[3,4-d]pyrimidin-2-amine

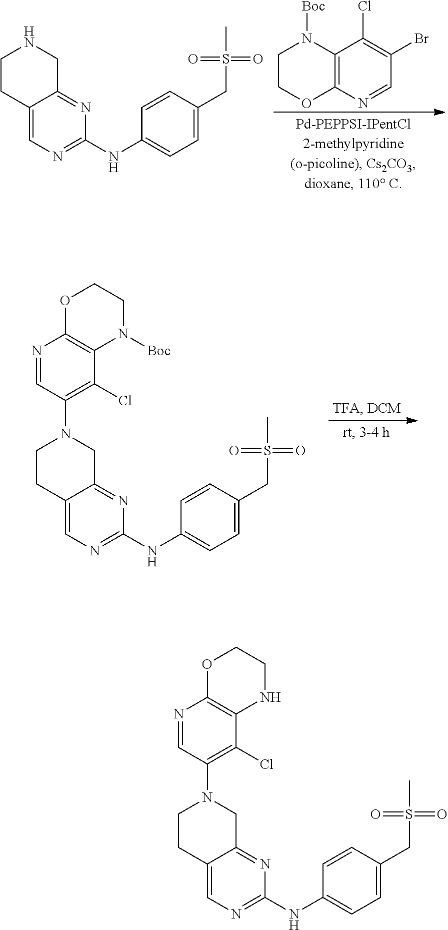

tert-butyl 8-chloro-7-(2-{[4-(methanesulfonylmethyl)phenyl]amino}-5H,6H,7H,8H-pyrido[3,4-d]pyrimidin-7-yl)-1H,2H,3H-pyrido[2,3-b][1,4]oxazine-1-carboxylate: To a stirred solution of tert-butyl 7-bromo-8-chloro-1H,2H,3H-pyrido[2,3-b][1,4]oxazine-1-carboxylate (248 mg; 0.61 mmol) in 1,4-dioxane (20.00 mL) were added N-[4-(methanesulfonylmethyl)phenyl]-5H,6H,7H,8H-pyrido[3,4-d]pyrimidin-2-amine (200 mg; 0.61 mmol), Pd-PEPPSI-IPentCl 2-methylpyridine (o-picoline) (28 mg; 0.031 mmol) and Cs₂CO₃ (630.0 mg; 1.84 mmol) in portions at room temperature under N₂ atmosphere. The resulting mixture was stirred for overnight at 100 degree C. under N₂ atmosphere. The residue was purified by silica gel column and eluted with 24% EtOAc in PE to afford tert-butyl 8-chloro-7-(2-{[4-(methanesulfonylmethyl)phenyl]amino}-5H,6H,7H,8H-pyrido[3,4-d]pyrimidin-7-yl)-1H,2H,3H-pyrido[2,3-b][1,4]oxazine-1-carboxylate (200 mg; 49.2%) as a yellow solid.

7-{8-chloro-1H,2H,3H-pyrido[2,3-b][1,4]oxazin-7-yl}-N-[4-(methanesulfonylmethyl)phenyl]-5H,6H,7H,8H-pyrido[3,4-d]pyrimidin-2-amine: To a stirred solution of tert-butyl 8-chloro-7-(2-{[4-(methanesulfonylmethyl) phenyl]amino}-5H,6H,7H,8H-pyrido[3,4-d]pyrimidin-7-yl)-1H,2H,3H-pyrido[2,3-b][1,4]oxazine-1-carboxylate (200 mg) in DCM (20.00 mL) was added TFA (5.00 mL) in portions at room temperature. The resulting mixture was stirred for 6 h at 25° C. under nitrogen atmosphere. The mixture was basified to pH [7] with saturated NaHCO₃ (aq.). The resulting mixture was extracted with DCM. The combined organic layers were washed with brine and dried over anhydrous Na₂SO₄. After filtration, the filtrate was concentrated under reduced pressure to afford the crude product. The crude product was purified by silica gel column and eluted with DCM/MeOH=19/1 to afford 7-{8-chloro-1H,2H,3H-pyrido[2,3-b][1,4]oxazin-7-yl}-N-[4-(methanesulfonylmethyl)phenyl]-5H,6H,7H,8H-pyrido[3,4-d]pyrimidin-2-amine (118.1 mg; 63.3%) as a yellow solid.

HPLC: 99.41% purity, RT=3.43 min. MS: m/z=487.00 [M+H]⁺. ¹H NMR (300 MHz, DMSO-d6) 9.65 (s, 1H), 8.36 (s, 1H), 7.88-7.70 (m, 2H), 7.32 (s, 1H), 7.29 (d, J=8.6 Hz, 2H), 6.11 (d, J=2.5 Hz, 1H), 4.38 (s, 2H), 4.31-4.19 (m, 2H), 4.10 (s, 2H), 3.34 (d, J=2.9 Hz, 2H), 3.26 (t, J=5.6 Hz, 2H), 2.87 (s, 3H), 2.82 (t, J=5.7 Hz, 2H).

### Example 64: Synthesis of Compound 64 7-{8-methyl-1H,2H,3H-pyrido[2,3-b][1,4]oxazin-7-yl}-N-{4-[(4-methylpiperazin-1-yl)methyl]phenyl}-5H,6H,7H,8H-pyrido[3,4-d]pyrimidin-2-amine

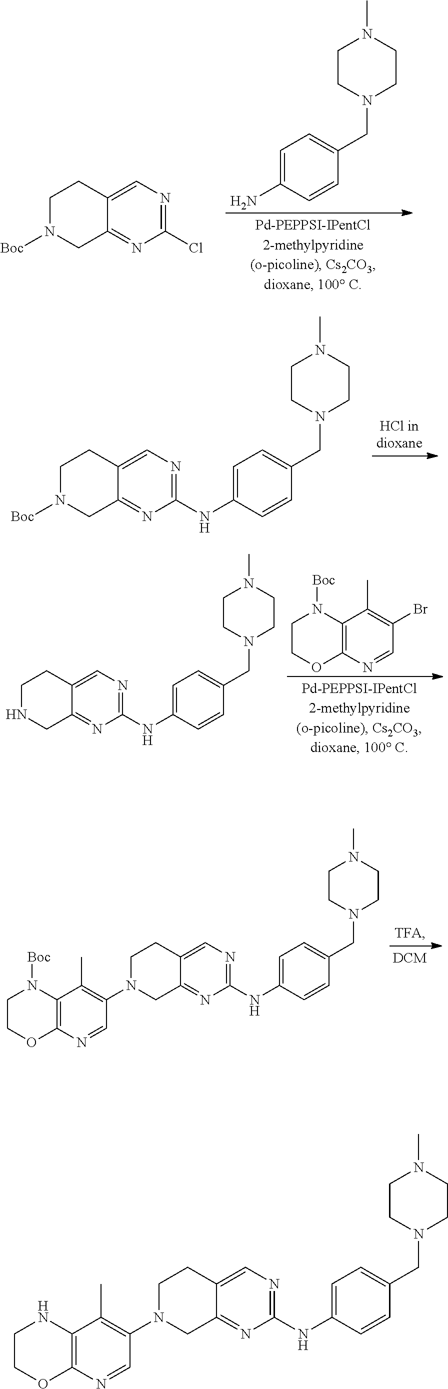

tert-butyl 2-({4-[(4-methylpiperazin-1-yl)methyl]phenyl}amino)-5H,6H,7H,8H-pyrido[3,4-d]pyrimidine-7-carboxylate: To a stirred solution of tert-butyl 2-chloro-5H,6H,7H,8H-pyrido[3,4-d]pyrimidine-7-carboxylate (300.00 mg; 0.96 mmol) and 4-[(4-methylpiperazin-1-yl) methyl]aniline (208.15 mg; 0.96 mmol) in 1,4-dioxane (15.00 mL) was added Pd-PEPPSI-IPentCl 2-methylpyridine (o-picoline) (85.18 mg; 0.10 mmol) and Cs₂CO₃ (660.69 mg; 1.93 mmol). The resulting mixture was stirred for 3 h at 100° C. under nitrogen atmosphere. The resulting mixture was concentrated under reduced pressure. The residue was purified by silica gel column chromatography and eluted with CH₂Cl₂/MeOH (9:1) to afford tert-butyl 2-({4-[(4-methylpiperazin-1-yl)methyl]phenyl}amino)-5H,6H,7H,8H-pyrido[3,4-d]pyrimidine-7-carboxylate (347.00 mg; 81.63%) as a yellow oil.

N-{4-[(4-methylpiperazin-1-yl) methyl]phenyl}-5H,6H,7H,8H-pyrido[3,4-d]pyrimidin-2-amine: To a stirred solution of tert-butyl 2-({4-[(4-methylpiperazin-1-yl)methyl]phenyl}amino)-5H,6H,7H,8H-pyrido[3,4-d]pyrimidine-7-carboxylate (330.00 mg; 0.63 mmol) in DCM (15.00 mL) was added TFA (3.00 mL) dropwise at room temperature. After stirring for 1 h at room temperature, the resulting mixture was concentrated under reduced pressure to afford N-{4-[(4-methylpiperazin-1-yl) methyl]phenyl}-5H,6H,7H,8H-pyrido[3,4-d]pyrimidin-2-amine (191.00 mg; crude product) as a yellow oil.

tert-butyl 8-methyl-7-[2-({4-[(4-methylpiperazin-1-yl)methyl]phenyl}amino)-5H,6H,7H,8H-pyrido[3,4-d]pyrimidin-7-yl]-1H,2H,3H-pyrido[2,3-b][1,4]oxazine-1-carboxylate: To a stirred solution of N-{4-[(4-methylpiperazin-1-yl)methyl]phenyl}-5H,6H,7H,8H-pyrido[3,4-d]pyrimidin-2-amine (90.00 mg; 0.25 mmol) and tert-butyl 7-bromo-8-methyl-1H,2H,3H-pyrido[2,3-b][1,4]oxazine-1-carboxylate (87.21 mg; 0.25 mmol) in 1,4-dioxane (10.00 mL) were added Pd-PEPPSI-IPentCl 2-methylpyridine (o-picoline) (22.28 mg; 0.03 mmol) and Cs₂CO₃ (172.79 mg; 0.50 mmol). The resulting mixture was stirred for 2 h at 100° C. under nitrogen atmosphere. The resulting mixture was concentrated under reduced pressure. The residue was purified by silica gel column chromatography and eluted with CH₂Cl₂/MeOH (5:1) to afford tert-butyl 8-methyl-7-[2-({4-[(4-methylpiperazin-1-yl)methyl]phenyl}amino)-5H,6H,7H,8H-pyrido[3,4-d]pyrimidin-7-yl]-1H,2H,3H-pyrido[2,3-b][1,4]oxazine-1-carboxylate (65.00 mg; 41.72%) as a yellow oil.

7-{8-methyl-1H,2H,3H-pyrido[2,3-b][1,4]oxazin-7-yl}-N-{4-[(4-methylpiperazin-1-yl)methyl]phenyl}-5H,6H,7H,8H-pyrido[3,4-d]pyrimidin-2-amine: To a stirred solution of tert-butyl 8-methyl-7-[2-({4-[(4-methylpiperazin-1-yl)methyl]phenyl}amino)-5H,6H,7H,8H-pyrido[3,4-d]pyrimidin-7-yl]-1H,2H,3H-pyrido[2,3-b][1,4]oxazine-1-carboxylate (101.00 mg; 0.15 mmol) in DCM (5.00 mL) was added TFA (1.00 mL) dropwise at room temperature. After stirring for 1 h at room temperature, the resulting mixture was concentrated under reduced pressure. The crude product was purified by Prep-HPLC with the following conditions: Column: XBridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 20% B to 47% B in 9 min., 47% B; Wave Length: 254 nm; RT1 (min.): 7, to afford 7-{8-methyl-1H,2H,3H-pyrido[2,3-b][1,4]oxazin-7-yl}-N-{4-[(4-methylpiperazin-1-yl)methyl]phenyl}-5H,6H,7H,8H-pyrido[3,4-d]pyrimidin-2-amine (12.10 mg; 15.82%) as a white solid.

HPLC: 98.57% purity, RT=2.71 min. MS: m/z=487.15 [M+H]⁺. ¹H NMR (300 MHz, Methanol-d4): 8.13 (d, J=2.1 Hz, 1H), 7.58-7.47 (m, 2H), 7.20-7.08 (m, 3H), 4.20 (t, J=4.3 Hz, 2H), 4.01 (s, 2H), 3.68 (d, J=1.6 Hz, 2H), 3.31 (t, J=4.4 Hz, 2H), 3.09 (s, 2H), 2.94 (s, 2H), 2.49 (s, 8H), 2.17 (d, J=1.1 Hz, 3H), 2.03 (d, J=1.3 Hz, 3H).

### Example 65: Synthesis of Compound 65 N-{3-[(dimethylamino)methyl]phenyl}-7-{8-methyl-1H,2H,3H-pyrido[2,3-b][1,4]oxazin-7-yl}-5H,6H,7H,8H-pyrido[3,4-d]pyrimidin-2-amine

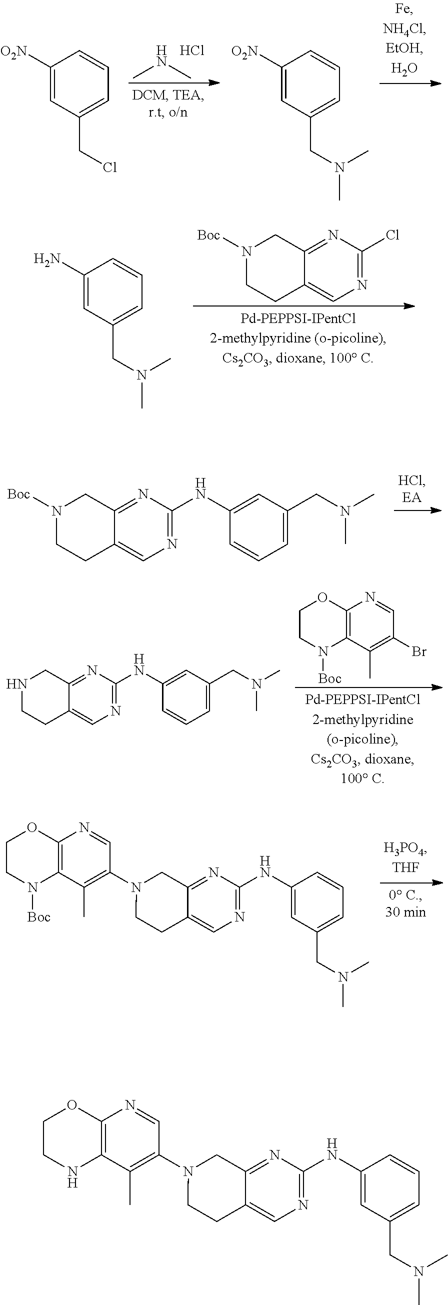

Dimethyl [(3-nitrophenyl) methyl] amine: To a stirred mixture of 1-(chloromethyl)-3-nitrobenzene (1.00 g; 5.54 mmol) and TEA (2.45 mL; 16.71 mmol) in DCM (15.00 mL) was added dimethylamine hydrochloride (0.95 g; 11.07 mmol) in portions at 0° C. The resulting mixture was stirred for overnight at room temperature under nitrogen atmosphere. Water was added to the reaction. The resulting mixture was extracted with EA (3×50 mL). The combined organic layers were concentrated under reduced pressure to afford dimethyl [(3-nitrophenyl) methyl] amine (1.00 g; crude product) as a brown oil.

3-[(dimethylamino) methyl]aniline: A solution of dimethyl [(3-nitrophenyl) methyl] amine (0.98 g; 5.41 mmol) and NH₄Cl (1.18 g; 20.96 mmol) and Fe (1.56 g; 26.46 mmol) in EtOH (6.00 mL) and H₂O (4.00 mL) was stirred for 2 h at 80° C. under N₂ atmosphere. The resulting mixture was concentrated under reduced pressure. The residue was purified by silica gel column chromatography and eluted with 27% MeOH in DCM. The residue was purified by reverse flash chromatography with the following conditions: column, C18 silica gel; mobile phase, MeOH in water, 45% to 5500 gradient in 10 min.; detector, UV 254 nm, to afford 3-[(dimethylamino) methyl]aniline (600.00 mg; 69.5%) as a colorless oil.

tert-butyl 2-({3-[(dimethylamino)methyl]phenyl}amino)-5H,6H,7H,8H-pyrido[3,4-d]pyrimidine-7-carboxylate: To a stirred mixture of tert-butyl 2-chloro-5H,6H,7H,8H-pyrido[3,4-d]pyrimidine-7-carboxylate (300.00 mg; 1.08 mmol) and 3-[(dimethylamino)methyl]aniline (300.00 mg; 1.88 mmol) in 1,4-dioxane (5.00 mL) was added Cs₂CO₃ (907.00 mg; 2.64 mmol) and Pd-PEPPSI-IPentCl 2-methylpyridine (o-picoline) (94.00 mg; 0.11 mmol) in portions at room temperature. The resulting mixture was stirred for 1 h at 100° C. under nitrogen atmosphere. The resulting mixture was concentrated under reduced pressure. The residue was purified by silica gel column chromatography and eluted with 5% MeOH in DCM to afford tert-butyl 2-({3-[(dimethylamino)methyl]phenyl}amino)-5H,6H,7H,8H-pyrido[3,4-d]pyrimidine-7-carboxylate (300.00 mg; 70.0%) as a brown oil.

N-{3-[(dimethylamino)methyl]phenyl}-5H,6H,7H,8H-pyrido[3,4-d]pyrimidin-2-amine: To a stirred mixture of tert-butyl 2-({3-[(dimethylamino)methyl]phenyl}amino)-5H,6H,7H,8H-pyrido[3,4-d]pyrimidine-7-carboxylate (290.00 mg; 0.73 mmol) in MeOH (1.00 mL) was added HCl (gas) in 1,4-dioxane (4.00 mL; 16.00 mmol) dropwise at 0° C. The resulting mixture was stirred for 1 h at room temperature under nitrogen atmosphere. The mixture was basified to pH 8 with Na₂CO₃ (aq.). The mixture was extracted with EA (3×20 mL). The combined organic layers were dried over anhydrous Na₂SO₄. After filtration, the filtrate was concentrated under reduced pressure to afford N-{3-[(dimethylamino) methyl]phenyl}-5H,6H,7H,8H-pyrido[3,4-d]pyrimidin-2-amine (210.00 mg; crude product) as a yellow solid.

tert-butyl 7-[2-({3-[(dimethylamino)methyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate: To a stirred mixture of N-{3-[(dimethylamino)methyl]phenyl}-5H,6H,7H,8H-pyrido[3,4-d]pyrimidin-2-amine (170.00 mg; 0.57 mmol) and tert-butyl 7-bromo-8-methyl-1H,2H,3H-pyrido[2,3-b][1,4]oxazine-1-carboxylate (300.00 mg; 0.90 mmol) in 1,4-dioxane (3.00 mL) was added Cs₂CO₃ (500.00 mg; 1.46 mmol) and Pd-PEPPSI-IPentCl 2-methylpyridine (o-picoline) (65.00 mg; 0.07 mmol) in portions at room temperature. The resulting mixture was stirred for overnight at 100° C. under nitrogen atmosphere. The resulting mixture was concentrated under reduced pressure. The residue was purified by silica gel column chromatography and eluted with 2% MeOH in DCM to afford tert-butyl 7-[2-({3-[(dimethylamino)methyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate (250.00 mg; 59.5%) as a yellow solid.

N-{3-[(dimethylamino)methyl]phenyl}-7-{8-methyl-1H,2H,3H-pyrido[2,3-b][1,4]oxazin-7-yl}-5H,6H,7H,8H-pyrido[3,4-d]pyrimidin-2-amine: To a stirred mixture of tert-butyl 7-[2-({3-[(dimethylamino)methyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate (230.00 mg; 0.31 mmol) in THF (1.00 mL) was added H₃PO₄ (3.00 mL; 54.50 mmol) dropwise at 0° C. The resulting mixture at was stirred for 30 min. at 0° C. under nitrogen atmosphere. The reaction was diluted with ice water at 0° C. The mixture was basified to pH 8 with Na₂CO₃. The resulting mixture was extracted with EA (3×30 mL). The combined organic layers were dried over anhydrous Na₂SO₄. After filtration, the filtrate was concentrated under reduced pressure. The crude product was purified by Prep-HPLC with the following conditions (Column: XBridge Shield RP18 OBD Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 24% to 52% in 9 min.; Wave Length: 254 nm; RT1 (min.): 7) to afford N-{3-[(dimethylamino)methyl]phenyl}-7-{8-methyl-1H,2H,3H-pyrido[2,3-b][1,4]oxazin-7-yl}-5H,6H,7H,8H-pyrido[3,4-d]pyrimidin-2-amine (63.40 mg; 44.4%) as a yellow solid.

HPLC: 93.9% purity, RT=2.99 min. MS: m/z=432.20 [M+H]⁺. ¹H NMR (300 MHz, DMSO-d6) 9.34 (s, 1H), 8.20 (s, 1H), 7.65-7.46 (m, 2H), 7.20-7.02 (m, 2H), 6.71 (d, J=7.6 Hz, 1H), 5.41 (d, J=2.7 Hz, 1H), 4.17-4.00 (m, 2H), 3.82 (s, 2H), 3.20 (d, J=9.2 Hz, 4H), 2.99 (t, J=5.7 Hz, 2H), 2.66 (t, J=5.7 Hz, 2H), 2.02 (s, 6H), 1.92 (s, 3H).

### Example 66: Synthesis of Compound 66 7-{8-methyl-1H,2H,3H-pyrido[2,3-b][1,4]oxazin-7-yl}-N-{3-[(4-methylpiperazin-1-yl)methyl]phenyl}-5H,6H,7H,8H-pyrido[3,4-d]pyrimidin-2-amine

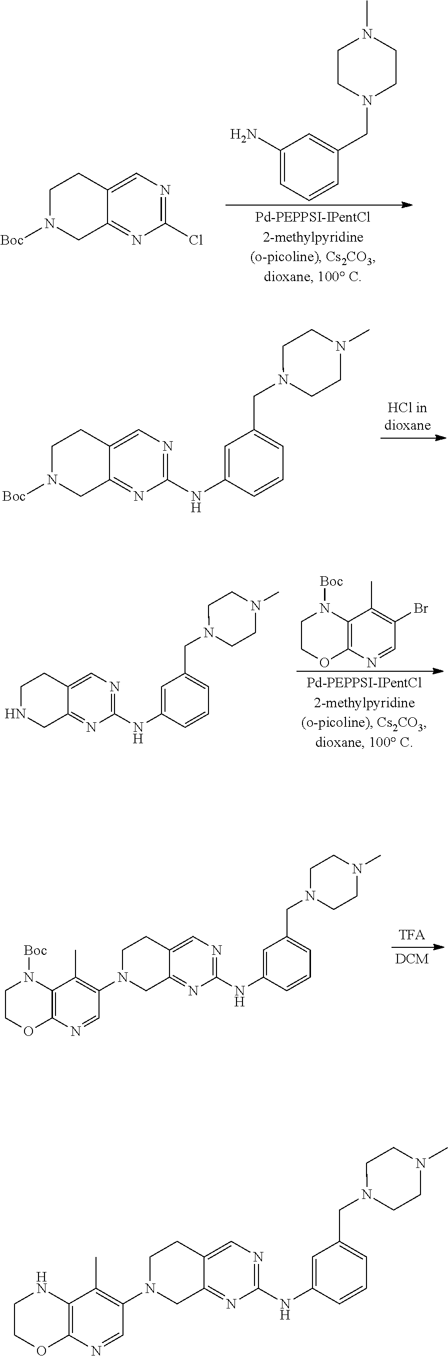

tert-butyl 2-({3-[(4-methylpiperazin-1-yl)methyl]phenyl}amino)-5H,6H,7H,8H-pyrido[3,4-d]pyrimidine-7-carboxylate: To a stirred mixture of 3-[(4-methylpiperazin-1-yl)methyl]aniline (249.78 mg; 1.16 mmol) and tert-butyl 2-chloro-5H,6H,7H,8H-pyrido[3,4-d]pyrimidine-7-carboxylate (300.00 mg; 0.96 mmol) in 1,4-dioxane (12.00 mL) were added Pd-PEPPSI-IPentCl 2-methylpyridine (o-picoline) (85.28 mg; 0.10 mmol) and Cs₂CO₃ (660.69 mg; 1.93 mmol) in portions at 25° C. The resulting mixture was stirred for 2 h at 80° C. under nitrogen atmosphere. The resulting mixture was concentrated under vacuum. The residue was purified by silica gel column chromatography and eluted with MeOH:DCM=1:20 to afford tert-butyl 2-({3-[(4-methylpiperazin-1-yl)methyl]phenyl}amino)-5H,6H,7H,8H-pyrido[3,4-d]pyrimidine-7-carboxylate (420.00 mg; 99.02%) as alight yellow solid.

N-{3-[(4-methylpiperazin-1-yl)methyl]phenyl}-5H,6H,7H,8H-pyrido[3,4-d]pyrimidin-2-amine: To a stirred mixture of tert-butyl 2-({3-[(4-methylpiperazin-1-yl)methyl]phenyl}amino)-5H,6H,7H,8H-pyrido[3,4-d]pyrimidine-7-carboxylate (400.00 mg; 0.91 mmol) in DCM (1.00 mL) and TFA (6.00 mL) at room temperature. After stirring for 1 h at room temperature, the reaction was quenched with H₂O (30 mL). The mixture was basified to pH=10 with Na₂CO₃ and extracted with CH₂Cl₂ (30 mL*5). The combined organic layers were washed with brine (150 mL) and dried over anhydrous Na₂SO₄. After filtration, the filtrate was concentrated under reduced pressure to afford N-{3-[(4-methylpiperazin-1-yl)methyl]phenyl}-5H,6H,7H,8H-pyrido[3,4-d]pyrimidin-2-amine (264.00 mg; crude product) as a yellow solid.

tert-butyl 8-methyl-7-[2-({3-[(4-methylpiperazin-1-yl)methyl]phenyl}amino)-5H,6H,7H,8H-pyrido[3,4-d]pyrimidin-7-yl]-1H,2H,3H-pyrido[2,3-b][1,4]oxazine-1-carboxylate: To a stirred mixture of tert-butyl 7-bromo-8-methyl-1H,2H,3H-pyrido[2,3-b][1,4]oxazine-1-carboxylate (140.35 mg; 0.41 mmol) and N-{3-[(4-methylpiperazin-1-yl)methyl]phenyl}-5H,6H,7H,8H-pyrido[3,4-d]pyrimidin-2-amine (130.00 mg; 0.35 mmol) in 1,4-dioxane (12.00 mL) were added Pd-PEPPSI-IPentCl 2-methylpyridine (o-picoline) (30.57 mg; 0.03 mmol) and Cs₂CO₃ (236.86 mg; 0.69 mmol) in portions at 25° C. The resulting mixture was stirred for 4 h at 100° C. under argon atmosphere. The resulting mixture was concentrated under vacuum. The residue was purified by silica gel column chromatography and eluted with MeOH:DCM=1:9 to afford tert-butyl 8-methyl-7-[2-({3-[(4-methylpiperazin-1-yl)methyl]phenyl}amino)-5H,6H,7H,8H-pyrido[3,4-d]pyrimidin-7-yl]-1H,2H,3H-pyrido[2,3-b][1,4]oxazine-1-carboxylate (191.00 mg; 93.50%) as a brown gum.

7-{8-methyl-1H,2H,3H-pyrido[2,3-b][1,4]oxazin-7-yl}-N-{3-[(4-methylpiperazin-1-yl)methyl]phenyl}-5H,6H,7H,8H-pyrido[3,4-d]pyrimidin-2-amine: To a stirred mixture of tert-butyl 8-methyl-7-[2-({3-[(4-methylpiperazin-1-yl)methyl]phenyl}amino)-5H,6H,7H,8H-pyrido[3,4-d]pyrimidin-7-yl]-1H,2H,3H-pyrido[2,3-b][1,4]oxazine-1-carboxylate (100.00 mg; 0.17 mmol) in TFE (4.00 mL) at room temperature. After stirring for 6 h at 100° C. under the microwave. The mixture was concentrated under reduced pressure. The crude product was purified by Prep-HPLC (Column: XBridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 20% B to 47% B in 9 min., 47% B; Wave Length: 254 nm; RTT (min.): 7; Number Of Runs: 2) to afford 7-{8-methyl-1H,2H,3H-pyrido[2,3-b][1,4]oxazin-7-yl}-N-{3-[(4-methylpiperazin-1-yl)methyl]phenyl}-5H,6H,7H,8H-pyrido[3,4-d]pyrimidin-2-amine (12.50 mg; 15.16%) as a light pink solid.

HPLC: 99.76% purity, RT=2.71 min. MS: m/z=487.30 [M+H]⁺. ¹H NMR (400 MHz, DMSO-d6): 9.47 (s, 1H), 8.33 (s, 1H), 7.72 (t, J=2.0 Hz, 1H), 7.62 (dd, J=7.9, 2.2 Hz, 1H), 7.29 (s, 1H), 7.18 (t, J=7.8 Hz, 1H), 6.83 (d, J=7.5 Hz, 1H), 5.54 (d, J=2.9 Hz, 1H), 4.19 (t, J=4.4 Hz, 2H), 3.95 (s, 2H), 3.41 (s, 2H), 3.30 (d, J=4.0 Hz, 2H), 3.13 (t, J=5.7 Hz, 2H), 2.79 (t, J=5.7 Hz, 2H), 2.34 (s, 8H), 2.13 (s, 3H), 2.04 (s, 3H).

### Example 67: Synthesis of Compound 67 2-{4-[(7-{8-methyl-1H,2H,3H-pyrido[2,3-b][1,4]oxazin-7-yl}-5H,6H,7H,8H-pyrido[3,4-d]pyrimidin-2-yl)amino]pyridin-2-yl}propan-2-ol

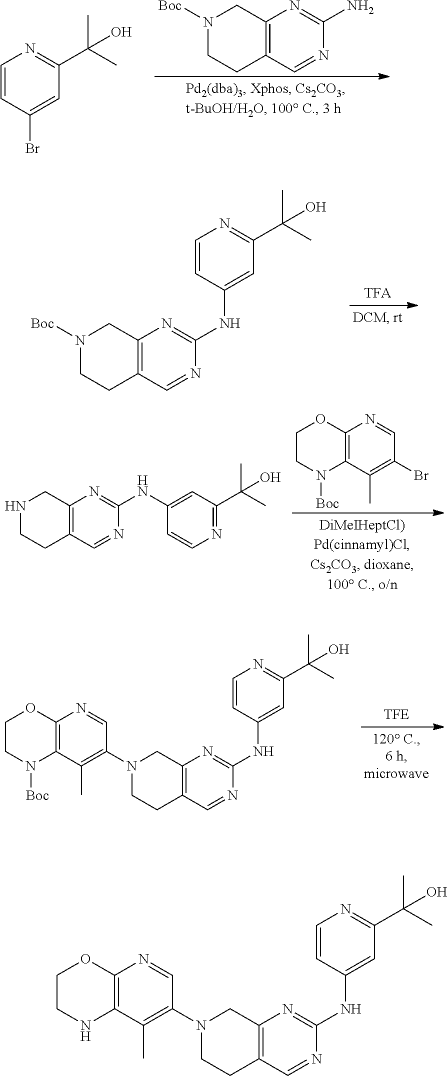

tert-butyl 2-{[2-(2-hydroxypropan-2-yl)pyridin-4-yl]amino}-5H,6H,7H,8H-pyrido[3,4-d]pyrimidine-7-carboxylate: To a solution of tert-butyl 2-amino-5H,6H,7H,8H-pyrido[3,4-d]pyrimidine-7-carboxylate (180.00 mg; 0.52 mmol) and 2-(4-bromopyridin-2-yl)propan-2-ol (118.00 mg; 0.52 mmol) in water (2.00 mL) and t-BuOH (10.00 mL) were added Cs₂CO₃ (356.00 mg; 1.04 mmol), Pd₂(dba)₃ (53.00 mg; 0.05 mmol) and Xphos (52.00 mg; 0.10 mmol). The resulting mixture was stirred for 3 h at 100° C. under nitrogen atmosphere. The resulting mixture was concentrated under reduced pressure. The residue was purified by silica gel column chromatography and eluted with DCM/MEOH (9:1) to afford tert-butyl 2-{[2-(2-hydroxypropan-2-yl)pyridin-4-yl]amino}-5H,6H,7H,8H-pyrido[3,4-d]pyrimidine-7-carboxylate (220.00 mg; 99.1%) as a yellow solid.

2-[4-({5H,6H,7H,8H-pyrido[3,4-d]pyrimidin-2-yl}amino)pyridin-2-yl]propan-2-ol: To a stirred solution of tert-butyl 2-{[2-(2-hydroxypropan-2-yl)pyridin-4-yl]amino}-5H,6H,7H,8H-pyrido[3,4-d]pyrimidine-7-carboxylate (200.00 mg; 0.47 mmol) in DCM (10.00 mL) was added TFA (0.20 mL) in portions at room temperature. Then the resulting mixture was stirred for overnight at room temperature. The resulting mixture was extracted with EtOAc and quenched with water. The combined organic layers were washed with brine and dried over anhydrous Na₂SO₄. After filtration, the filtrate was concentrated under reduced pressure to afford 2-[4-({5H,6H,7H,8H-pyrido[3,4-d]pyrimidin-2-yl}amino)pyridin-2-yl]propan-2-ol (150.00 mg; 92.1%) as a yellow oil.

tert-butyl 7-(2-{[2-(2-hydroxypropan-2-yl)pyridin-4-yl]amino}-5H,6H,7H,8H-pyrido[3,4-d]pyrimidin-7-yl)-8-methyl-1H,2H,3H-pyrido[2,3-b][1,4]oxazine-1-carboxylate: To a solution of 2-[4-({5H,6H,7H,8H-pyrido[3,4-d]pyrimidin-2-yl}amino)pyridin-2-yl]propan-2-ol (130.00 mg; 0.37 mmol) and tert-butyl 7-bromo-8-methyl-1H,2H,3H-pyrido[2,3-b][1,4]oxazine-1-carboxylate (125.00 mg; 0.38 mmol) in 1,4-dioxane (2.00 mL) were added Cs₂CO₃ (256.00 mg; 0.75 mmol) and DiMeIHeptCl Pd(cinnamyl)Cl (42.00 mg; 0.04 mmol). The resulting mixture was stirred for overnight at 100° C. under nitrogen atmosphere. The resulting mixture was concentrated under reduced pressure. The residue was purified by silica gel column chromatography and eluted with DCM/MEOH (6:1) to afford tert-butyl 7-(2-{[2-(2-hydroxypropan-2-yl)pyridin-4-yl]amino}-5H,6H,7H,8H-pyrido[3,4-d]pyrimidin-7-yl)-8-methyl-1H,2H,3H-pyrido[2,3-b][1,4]oxazine-1-carboxylate (60.00 mg; 26.9%) as a yellow solid.

2-{4-[(7-{8-methyl-1H,2H,3H-pyrido[2,3-b][1,4]oxazin-7-yl}-5H,6H,7H,8H-pyrido[3,4-d]pyrimidin-2-yl)amino]pyridin-2-yl}propan-2-ol: To a stirred solution of tert-butyl 7-(2-{[2-(2-hydroxypropan-2-yl)pyridin-4-yl]amino}-5H,6H,7H,8H-pyrido[3,4-d]pyrimidin-7-yl)-8-methyl-1H,2H,3H-pyrido[2,3-b][1,4]oxazine-1-carboxylate (50.00 mg; 0.08 mmol) in TFE (10.00 mL) was stirred for 6 h at 120° C. The resulting mixture was concentrated under reduced pressure. The crude product was purified by Prep-HPLC with the following conditions (Column: XBridge Prep OBD C18 Column, 30*150 mm, 5 μm; Mobile Phase A: water (10 mmol/L NH₄HCO₃+0.05% NH₄OH), Mobile Phase B: ACN; Flow rate: 60 m/min.; Gradient: 20% to 47% in 9 min.; Wave Length: 254 nm; RT1 (min.): 7; Number Of Runs: 2) to afford 2-{4-[(7-{8-methyl-1H,2H,3H-pyrido[2,3-b][1,4]oxazin-7-yl}-5H,6H,7H,8H-pyrido[3,4-d]pyrimidin-2-yl)amino]pyridin-2-yl}propan-2-ol (12.90 mg; 34.9%) as a white solid.

HPLC: 97.9% purity, RT=2.80 min. MS: m/z=434.00 [M+H]⁺. ¹H NMR (300 MHz, DMSO-d6) 10.71 (s, 1H), 8.43 (s, 1H), 8.22 (d, J=6.4 Hz, 1H), 8.03 (d, J=8.7 Hz, 1H), 7.92-7.72 (m, 1H), 7.18 (s, 1H), 5.70 (s, 1H), 5.45 (s, 1H), 4.07 (t, J=4.5 Hz, 2H), 3.94 (s, 2H), 3.17 (s, 2H), 3.02 (d, J=6.3 Hz, 2H), 2.76 (s, 2H), 1.92 (s, 3H), 1.38 (s, 6H).

### Example 68: Synthesis of Compound 68 6-methyl-2-[(6-{8-methyl-1H,2H,3H-pyrido[2,3-b][1,4]oxazin-7-yl}-5,6,7,8-tetrahydro-2,6-naphthyridin-3-yl)amino]-4H,5H,6H,7H,8H-pyrazolo[1,5-d][1,4]diazepin-7-one

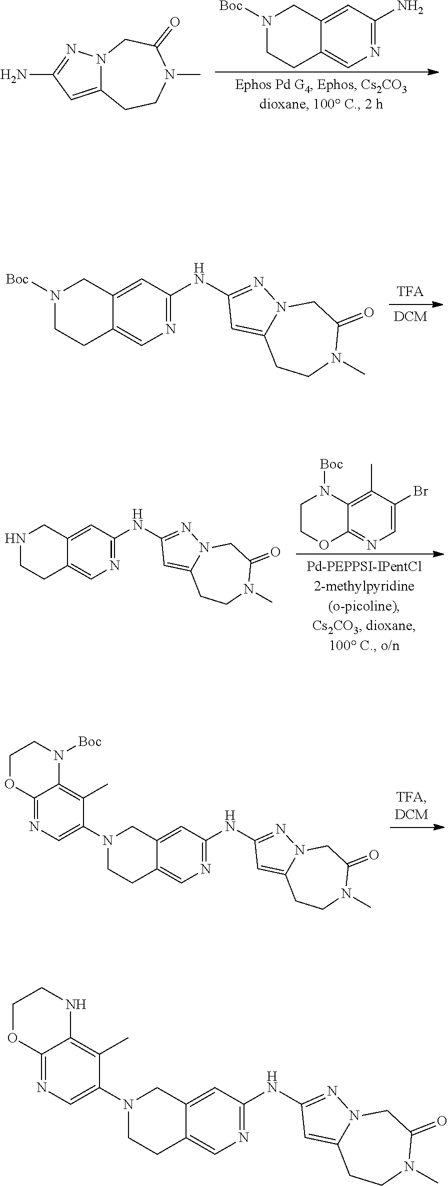

tert-butyl 7-({6-methyl-7-oxo-4H,5H,6H,7H,8H-pyrazolo[1,5-d][1,4]diazepin-2-yl}amino)-1,2,3,4-tetrahydro-2,6-naphthyridine-2-carboxylate: To a stirred solution of 2-amino-6-methyl-4H,5H,6H,7H,8H-pyrazolo[1,5-d][1,4]diazepin-7-one (560.00 mg; 2.91 mmol) and tert-butyl 7-chloro-1,2,3,4-tetrahydro-2,6-naphthyridine-2-carboxylate (918.00 mg; 3.35 mmol) in 1,4-dioxane (10.00 mL) were added EPhos Pd G₄ (286.00 mg; 0.30 mmol) and Ephos (333.00 mg; 0.59 mmol) and Cs₂CO₃ (3.10 g; 9.04 mmol) in portions at room temperature under N₂ atmosphere. The resulting mixture was stirred for overnight at 100° C. under N₂ atmosphere. The resulting mixture was diluted with water (50 mL), extracted with EtOAc (3×50 mL). The combined organic layers were washed with brine (50 mL) and dried over anhydrous Na₂SO₄. The resulting mixture was concentrated under reduced pressure. The residue was purified by silica gel column and eluted with 10% MeOH in DCM to afford tert-butyl 7-({6-methyl-7-oxo-4H,5H,6H,7H,8H-pyrazolo[1,5-d][1,4]diazepin-2-yl}amino)-1,2,3,4-tetrahydro-2,6-naphthyridine-2-carboxylate (1.12 g; 90.5%) as a yellow solid.

6-methyl-2-[(5,6,7,8-tetrahydro-2,6-naphthyridin-3-yl)amino]-4H,5H,6H,7H,8H-pyrazolo[1,5-d][1,4]diazepin-7-one: To a stirred solution of tert-butyl 7-({6-methyl-7-oxo-4H,5H,6H,7H,8H-pyrazolo[1,5-d][1,4]diazepin-2-yl}amino)-1,2,3,4-tetrahydro-2,6-naphthyridine-2-carboxylate (1.10 g; 2.59 mmol) in DCM (6.00 mL) was added TFA (2.00 mL) dropwise at 0° C. under N₂ atmosphere. The resulting mixture was stirred for 1 h at room temperature under N₂ atmosphere. The resulting mixture was concentrated under reduced pressure. The residue was purified by silica gel column and eluted with 37% ACN in water to afford 6-methyl-2-[(5,6,7,8-tetrahydro-2,6-naphthyridin-3-yl)amino]-4H,5H,6H,7H,8H-pyrazolo[1,5-d][1,4]diazepin-7-one (780.00 mg; 95.3%) as a yellow solid.

tert-butyl 8-methyl-7-[7-({6-methyl-7-oxo-4H,5H,6H,7H,8H-pyrazolo[1,5-d][1,4]diazepin-2-yl}amino)-1,2,3,4-tetrahydro-2,6-naphthyridin-2-yl]-1H,2H,3H-pyrido[2,3-b][1,4]oxazine-1-carboxylate: To a stirred solution of 6-methyl-2-[(5,6,7,8-tetrahydro-2,6-naphthyridin-3-yl) amino]-4H,5H,6H,7H,8H-pyrazolo[1,5-d][1,4]diazepin-7-one (250.00 mg; 0.79 mmol) and tert-butyl 7-bromo-8-methyl-1H,2H,3H-pyrido[2,3-b][1,4]oxazine-1-carboxylate (342.00 mg; 1.03 mmol) in 1,4-dioxane (5.00 mL) was added Cs₂CO₃ (784.00 mg; 2.29 mmol) and Pd-PEPPSI-IPentCl 2-methylpyridine (o-picoline) (68.00 mg; 0.08 mmol) in portions at room temperature under N₂ atmosphere. The resulting mixture was stirred for 1 h at 100° C. under N₂ atmosphere. The resulting mixture was diluted with water (50 mL), extracted with EtOAc (3×50 mL). The combined organic layers were washed with brine (50 mL) and dried over anhydrous Na₂SO₄. The resulting mixture was concentrated under reduced pressure. The residue was purified by silica gel column and eluted with 10% MeOH in DCM to afford tert-butyl 8-methyl-7-[7-({6-methyl-7-oxo-4H,5H,6H,7H,8H-pyrazolo[1,5-d][1,4]diazepin-2-yl}amino)-1,2,3,4-tetrahydro-2,6-naphthyridin-2-yl]-1H,2H,3H-pyrido[2,3-b][1,4]oxazine-1-carboxylate (360.00 mg; 69.6%) as a yellow solid.

6-methyl-2-[(6-{8-methyl-1H,2H,3H-pyrido[2,3-b][1,4]oxazin-7-yl}-5,6,7,8-tetrahydro-2,6-naphthyridin-3-yl) amino]-4H,5H,6H,7H,8H-pyrazolo[1,5-d][1,4]diazepin-7-one: To a stirred solution of tert-butyl 8-methyl-7-[7-({6-methyl-7-oxo-4H,5H,6H,7H,8H-pyrazolo[1,5-d][1,4]diazepin-2-yl}amino)-1,2,3,4-tetrahydro-2,6-naphthyridin-2-yl]-1H,2H,3H-pyrido[2,3-b][1,4]oxazine-1-carboxylate (350.00 mg; 0.54 mmol) in DCM (6.00 mL) was added TFA (2.00 mL) dropwise at 0° C. under N₂ atmosphere. The resulting mixture was stirred for 2 h at room temperature under N₂ atmosphere. The resulting mixture was concentrated under reduced pressure. The residue was purified by Prep-HPLC with the following condition: Column: XBridge Shield RP18 OBD Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 17% to 47% in 9 min.; Wave Length: 254 nm; RTT (min.): 7, to afford 6-methyl-2-[(6-{8-methyl-1H,2H,3H-pyrido[2,3-b][1,4]oxazin-7-yl}-5,6,7,8-tetrahydro-2,6-naphthyridin-3-yl)amino]-4H,5H,6H,7H,8H-pyrazolo[1,5-d][1,4]diazepin-7-one (81.10 mg; 32.6%) as a white solid.

HPLC: 99.2% purity, RT=2.44 min. MS: m/z=461.20[M+H]⁺. ¹H NMR (300 MHz, DMSO-d6) 8.97 (s, 1H), 7.93 (s, 1H), 7.26 (s, 1H), 6.99 (s, 1H), 6.18 (s, 1H), 5.49 (s, 1H), 4.91 (s, 2H), 4.18 (s, 2H), 3.97 (s, 2H), 3.86-3.76 (m, 2H), 3.29 (s, 2H), 3.03 (dd, J=13.2, 6.6 Hz, 4H), 2.94 (s, 3H), 2.80 (d, J=5.4 Hz, 2H), 2.02 (s, 3H).

### Example 69: Synthesis of Compound 69 N-[4-(methanesulfonylmethyl)phenyl]-7-{8-methyl-1H,2H,3H-pyrido[2,3-b][1,4]oxazin-7-yl}-5H,6H,7H,8H-pyrido[3,4-d]pyrimidin-2-amine

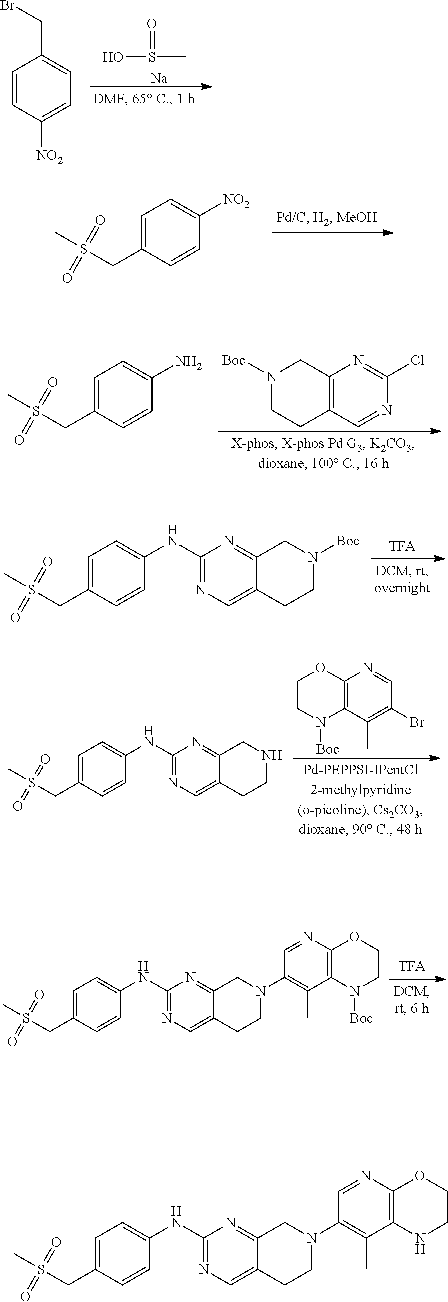

1-(methanesulfonylmethyl)-4-nitrobenzene: To a stirred solution of 1-(bromomethyl)-4-nitrobenzene (500.00 mg; 2.19 mmol) in DMF (15.00 mL) was added sodium methanesulfinate (362.80 mg; 3.52 mmol). Then the resulting mixture was stirred for additional 2 h at 65° C. The reaction was quenched by the addition of H₂O at room temperature and extracted by EA. The combined organic layers were washed with brine and dried over anhydrous Na₂SO₄. After filtration, the filtrate was concentrated under reduced pressure to afford 1-(methanesulfonylmethyl)-4-nitrobenzene (420.00 mg; crude product) as a white solid.

4-(methanesulfonylmethyl) aniline: To a solution of 1-(methanesulfonylmethyl)-4-nitrobenzene (450.00 mg; 1.88 mmol) in MeOH (50.00 mL) was added Palladium on activated carbon (21.1 mg; 0.18 mmol). The mixture was hydrogenated at room temperature for 2 h under hydrogen atmosphere. The resulting mixture was filtered and the filter cake was washed with 30 mL of EA. The filtrate was concentrated under reduced pressure. The resulting filter residue was added water and quenched by the addition of HCl (2 mL) at room temperature. The filtrate was concentrated under reduced pressure to afford 4-(methanesulfonylmethyl) aniline (430.00 mg; crude product) as a brown solid.

tert-butyl 2-{[4-(methanesulfonylmethyl)phenyl]amino}-5H,6H,7H,8H-pyrido[3,4-d]pyrimidine-7-carboxylate: To a solution of tert-butyl 2-chloro-5H,6H,7H,8H-pyrido[3,4-d]pyrimidine-7-carboxylate (200.0 g; 0.73 mmol) and 4-(methanesulfonylmethyl)aniline (146.70 mg; 0.73 mmol) in 1,4-dioxane (5.00 mL) were added Cs₂CO₃ (500.00 mg; 1.46 mmol), XPhos Pd G₃ (32.00 mg; 0.036 mmol) and Xphos (36.00 mg; 0.072 mmol). The resulting mixture was stirred for overnight at 100° C. under nitrogen atmosphere. The resulting mixture was concentrated under vacuum. The residue was purified by silica gel column chromatography and eluted with DCM/MeOH (9:1) to afford tert-butyl 2-{[4-(methanesulfonylmethyl)phenyl]amino}-5H,6H,7H,8H-pyrido[3,4-d]pyrimidine-7-carboxylate (200.00 mg; 62.3%) as a yellow solid.

N-[4-(methanesulfonylmethyl)phenyl]-5H,6H,7H,8H-pyrido[3,4-d]pyrimidin-2-amine: To a stirred solution of tert-butyl 2-{[4-(methanesulfonylmethyl)phenyl]amino}-5H,6H,7H,8H-pyrido[3,4-d]pyrimidine-7-carboxylate (300.00 mg; 0.66 mmol) in DCM (30.00 mL) was added TFA (7.50 mL) in portions at room temperature. The resulting mixture was stirred for 4 h at 25° C. under nitrogen atmosphere. The mixture was basified to pH [14] with saturated NaOH (aq.), the filter cake was washed with H₂O and dried under reduced pressure to afford N-[4-(methanesulfonylmethyl)phenyl]-5H,6H,7H,8H-pyrido[3,4-d]pyrimidin-2-amine (210.00 mg; crude product) as a yellow solid.

tert-butyl 7-(2-{[4-(methanesulfonylmethyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate: To a solution of N-[4-(methanesulfonylmethyl)phenyl]-5H,6H,7H,8H-pyrido[3,4-d]pyrimidin-2-amine (80.00 mg; 0.25 mmol) and tert-butyl 7-bromo-8-methyl-1H,2H,3H-pyrido[2,3-b][1,4]oxazine-1-carboxylate (87.1 mg; 0.25 mmol) in 1,4-dioxane (6.00 mL) were added Cs₂CO₃ (170.00 mg; 0.50 mmol) and Pd-PEPPSI-IPentCl 2-methylpyridine (o-picoline) (10.10 mg; 0.012 mmol). The resulting mixture was stirred for 48 h at 90° C. under nitrogen atmosphere. The resulting mixture was concentrated under reduced pressure. The residue was purified by silica gel column chromatography and eluted with DCM/MeOH (19:1) to afford tert-butyl 7-(2-{[4-(methanesulfonylmethyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (25.00 mg; 50.8%) as a yellow solid.

N-[4-(methanesulfonylmethyl)phenyl]-7-{8-methyl-1H,2H,3H-pyrido[2,3-b][1,4]oxazin-7-yl}-5H,6H,7H,8H-pyrido[3,4-d]pyrimidin-2-amine: To a stirred solution of tert-butyl 7-(2-{[4-(methanesulfonylmethyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (80.00 mg; 0.14 mmol) in DCM (8.00 mL) was added TFA (2.00 mL) in portions at room temperature. The resulting mixture was stirred for 6 h at 25° C. under nitrogen atmosphere. The mixture was basified to pH [7] with saturated NaHCO₃ (aq.). The resulting mixture was extracted with DCM. The combined organic layers were washed with brine and dried over anhydrous Na₂SO₄. After filtration, the filtrate was concentrated under reduced pressure to afford the crude. The crude was purified by silica gel column chromatography and eluted with DCM/MeOH (19:1) to afford N-[4-(methanesulfonylmethyl)phenyl]-7-{8-methyl-1H,2H,3H-pyrido[2,3-b][1,4]oxazin-7-yl}-5H,6H,7H,8H-pyrido[3,4-d]pyrimidin-2-amine (80.60 mg; 66.3%) as a yellow solid.

HPLC: 99.2% purity, RT=2.94 min. MS: m/z=467.00 [M+H]⁺. ¹H NMR (300 MHz, DMSO-d6) 9.62 (s, 1H), 8.35 (s, 1H), 7.77 (d, J=8.6 Hz, 2H), 7.36-7.25 (m, 3H), 5.51 (s, 1H), 4.37 (s, 2H), 4.19 (t, J=4.4 Hz, 2H), 3.97 (s, 2H), 3.33 (s, 2H), 3.12 (t, J=5.6 Hz, 2H), 2.86 (s, 3H), 2.78 (s, 2H), 2.04 (s, 3H).

### Example 70: Synthesis of Compound 70 N-(7-{8-methyl-1H,2H,3H-pyrido[2,3-b][1,4]oxazin-7-yl}-5H,6H,7H,8H-pyrido[3,4-d]pyrimidin-2-yl)-1-[(1-methyl-1H-pyrazol-4-yl)methyl]piperidin-4-amine

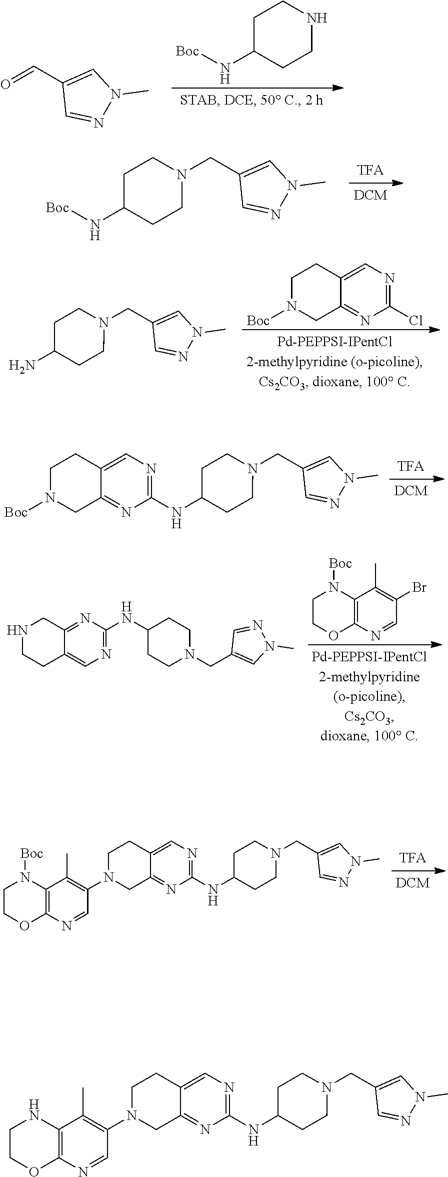

tert-butyl N-{1-[(1-methyl-1H-pyrazol-4-yl)methyl]piperidin-4-yl}carbamate: To a stirred mixture of 1-methyl-TH-pyrazole-4-carbaldehyde (1.00 g; 8.63 mmol) and tert-butyl N-(piperidin-4-yl)carbamate (2.00 g; 9.49 mmol) in DCE (20.00 mL) was added STAB (3.85 g; 17.25 mmol) in portions at 25° C. The resulting mixture was stirred for 2 h at 50° C. under nitrogen atmosphere. The reaction was quenched with NaHCO₃ (aq.) at room temperature. The resulting mixture was extracted with EA (3×30 mL). The combined organic layers were washed with brine and dried over anhydrous MgSO₄. After filtration, the filtrate was concentrated under reduced pressure. The residue was purified by C18 column chromatography and eluted with ACN: H₂O=1:1 to afford tert-butyl N-{1-[(1-methyl-1H-pyrazol-4-yl) methyl] piperidin-4-yl}carbamate (2.00 g; 78.0%) as a yellow oil.

1-[(1-methyl-1H-pyrazol-4-yl) methyl] piperidin-4-amine: To a stirred mixture of tert-butyl N-{1-[(1-methyl-1H-pyrazol-4-yl) methyl] piperidin-4-yl}carbamate (2 g; 6.7 mmol) in DCM (20 mL) was added TFA (5 mL) dropwise at 25° C. The resulting mixture was stirred for 2 h at 25° C. under nitrogen atmosphere. The resulting mixture was concentrated under vacuum. The crude product was purified by Prep-HPLC with the following conditions (Column: Xselect CSH C18 OBD Column 30*150 mm, 5 μm; Mobile Phase A: water (0.1% FA), Mobile Phase B: ACN; Flow rate: 60 mL/min.; Gradient: 5% B to 25% B in 8 min., 25% B; Wave Length: 254/220 nm; RT1 (min.): 6.8) to afford 1-[(1-methyl-1H-pyrazol-4-yl)methyl]piperidin-4-amine (200.00 mg; 7.1%) as a yellow oil.

tert-butyl 2-({1-[(1-methyl-1H-pyrazol-4-yl)methyl]piperidin-4-yl}amino)-5H,6H,7H,8H-pyrido[3,4-d]pyrimidine-7-carboxylate: To a stirred mixture of 1-[(1-methyl-1H-pyrazol-4-yl)methyl] piperidin-4-amine (168.00 mg; 0.80 mmol) and tert-butyl 2-chloro-5H,6H,7H,8H-pyrido[3,4-d]pyrimidine-7-carboxylate (250.00 mg; 0.80 mmol) in 1,4-dioxane (10.00 mL) were added Pd-PEPPSI-IPentCl 2-methylpyridine (o-picoline) (72.00 mg; 0.08 mmol) and Cs₂CO₃ (551.00 mg; 1.61 mmol) in portions at 25° C. The resulting mixture was stirred for 3 h at 100° C. under nitrogen atmosphere. The resulting mixture was concentrated under vacuum. The residue was purified by silica gel column chromatography and eluted with MeOH:DCM=1:20 to afford tert-butyl 2-({1-[(1-methyl-1H-pyrazol-4-yl)methyl]piperidin-4-yl}amino)-5H,6H,7H,8H-pyrido[3,4-d]pyrimidine-7-carboxylate (300.00 mg; 74.5%) as a yellow solid.

1-[(1-methyl-1H-pyrazol-4-yl)methyl]piperidin-4-amine: To a stirred mixture of tert-butyl 2-({1-[(1-methyl-1H-pyrazol-4-yl)methyl]piperidin-4-yl}amino)-5H,6H,7H,8H-pyrido[3,4-d]pyrimidine-7-carboxylate (200.00 mg; 0.67 mmol) in DCM (2.00 mL) was added TFA (0.50 mL) dropwise at 25° C. The resulting mixture was stirred for 2 h at 25° C. under nitrogen atmosphere. The resulting mixture was concentrated under vacuum. The crude product was purified by Prep-HPLC with the following conditions (Column: Xselect CSH C18 OBD Column 30*150 mm, 5 μm, n; Mobile Phase A: water (0.1% FA), Mobile Phase B: ACN; Flow rate: 60 m/min.; Gradient: 5% B to 25% B in 8 min., 25% B; Wave Length: 254/220 nm; RT1 (min.): 6.8) to afford 1-[(1-methyl-1H-pyrazol-4-yl)methyl]piperidin-4-amine (20.00 mg; 7.1%) as a yellow oil.

tert-butyl 2-({1-[(1-methyl-1H-pyrazol-4-yl)methyl]piperidin-4-yl}amino)-5H,6H,7H,8H-pyrido[3,4-d]pyrimidine-7-carboxylate: To a stirred mixture of 1-[(1-methyl-1H-pyrazol-4-yl)methyl] piperidin-4-amine (168.00 mg; 0.80 mmol) and tert-butyl 2-chloro-5H,6H,7H,8H-pyrido[3,4-d]pyrimidine-7-carboxylate (250.00 mg; 0.80 mmol) in 1,4-dioxane (10.00 mL) were added Pd-PEPPSI-IPentCl 2-methylpyridine (o-picoline) (72.00 mg; 0.08 mmol) and Cs₂CO₃ (551.00 mg; 1.61 mmol) in portions at 25° C. The resulting mixture was stirred for 3 h at 100° C. under nitrogen atmosphere. The resulting mixture was concentrated under vacuum. The residue was purified by silica gel column chromatography and eluted with MeOH:DCM=1:20 to afford tert-butyl 2-({1-[(1-methyl-1H-pyrazol-4-yl) methyl]piperidin-4-yl}amino)-5H,6H,7H,8H-pyrido[3,4-d]pyrimidine-7-carboxylate (300.00 mg; 74.5%) as a yellow solid.

N-(7-{8-methyl-1H,2H,3H-pyrido[2,3-b][1,4]oxazin-7-yl}-5H,6H,7H,8H-pyrido[3,4-d]pyrimidin-2-yl)-1-[(1-methyl-1H-pyrazol-4-yl)methyl]piperidin-4-amine: To a stirred mixture of tert-butyl 8-methyl-7-[2-({1-[(1-methyl-1H-pyrazol-4-yl)methyl] piperidin-4-yl}amino)-5H,6H,7H,8H-pyrido[3,4-d]pyrimidin-7-yl]-1H,2H,3H-pyrido[2,3-b][1,4]oxazine-1-carboxylate (80.00 mg; 0.10 mmol) in DCM (4.00 mL) was added TFA (1.00 mL) dropwise at 25° C. The resulting mixture was stirred for 2 h at 25° C. under nitrogen atmosphere. The resulting mixture was concentrated under vacuum. The crude product was purified by Prep-HPLC with the following conditions (Column: XBridge Shield RP18 OBD Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 16% B to 44% B in 9 min., 44% B; Wave Length: 254 nm; RT1 (min.): 7) to afford N-(7-{8-methyl-1H,2H,3H-pyrido[2,3-b][1,4]oxazin-7-yl}-5H,6H,7H,8H-pyrido[3,4-d]pyrimidin-2-yl)-1-[(1-methyl-1H-pyrazol-4-yl)methyl]piperidin-4-amine (19.90 mg; 41.6%) as a yellow solid.

HPLC: 95.1% purity, RT=2.52 min. MS: m/z=476.25 [M+H]⁺. ¹H NMR (400 MHz, DMSO-d6) 8.10 (s, 1H), 7.53 (s, 1H), 7.25 (d, J=15.5 Hz, 2H), 6.78 (d, J=8.0 Hz, 1H), 5.49 (d, J=2.7 Hz, 1H), 4.21-4.15 (m, 2H), 3.79 (d, J=7.8 Hz, 5H), 3.63 (dd, J=8.9, 5.1 Hz, 1H), 3.31 (d, J=7.7 Hz, 4H), 3.05 (t, J=5.7 Hz, 2H), 2.78 (d, J=11.1 Hz, 2H), 2.68 (d, J=5.7 Hz, 2H), 2.03 (s, 1H), 2.01 (s, 2H), 1.94 (t, J=11.2 Hz, 2H), 1.84-1.76 (m, 2H), 1.44 (qd, J=11.9, 3.8 Hz, 2H).

### Example 71: Synthesis of Compound 71 methyl N-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}carbamate

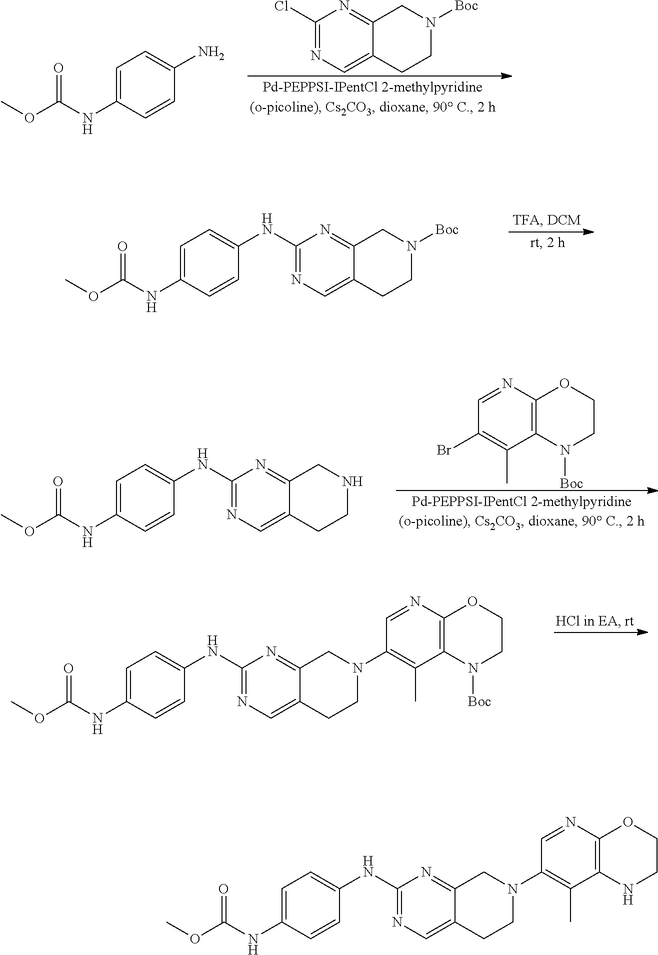

2-({4-[(methoxycarbonyl)amino]phenyl}amino)-5H,6H,7H,8H-pyrido[3,4-d]pyrimidine-7-carboxylate: To a stirred solution of tert-butyl 2-chloro-5H,6H,7H,8H-pyrido[3,4-d]pyrimidine-7-carboxylate (400.00 mg; 1.46 mmol) and methyl N-(4-aminophenyl)carbamate (291.00 mg; 1.75 mmol) in 1,4-dioxane (15.00 mL) were added Cs₂CO₃ (1.50 g; 4.37 mmol) and Pd-PEPPSI-IPentCl 2-methylpyridine (o-picoline) (129.00 mg; 0.15 mmol) in portions at room temperature under N₂ atmosphere. The resulting mixture was stirred for 2 h at 90° C. under N₂ atmosphere. The resulting mixture was concentrated under reduced pressure. The residue was purified by C18 column and eluted with 48% ACN in H₂O to afford tert-butyl 2-({4-[(methoxycarbonyl)amino]phenyl}amino)-5H,6H,7H,8H-pyrido[3,4-d]pyrimidine-7-carboxylate (380.00 mg; 60.6%) as a yellow solid.

methyl N-[4-({5H,6H,7H,8H-pyrido[3,4-d]pyrimidin-2-yl}amino)phenyl]carbamate: To a stirred solution of tert-butyl 2-({4-[(methoxycarbonyl)amino]phenyl}amino)-5H,6H,7H,8H-pyrido[3,4-d]pyrimidine-7-carboxylate (250.00 mg; 0.58 mmol) in DCM (8.00 mL) was added TFA (2.00 mL; 25.58 mmol) in portions at room temperature under N₂ atmosphere. The resulting mixture was stirred for 2 h at 25° C. under N₂ atmosphere. The resulting mixture was concentrated under reduced pressure, diluted with EtOAc (5 mL), basified to pH 9 with NaHCO₃, extracted with EtOAc (4×100 mL). The resulting mixture was concentrated under reduced pressure to afford methyl N-[4-({5H,6H,7H,8H-pyrido[3,4-d]pyrimidin-2-yl}amino)phenyl]carbamate (210.00 mg; 93.8%) as a yellow solid.

tert-butyl 7-[2-({4-[(methoxycarbonyl)amino]phenyl}amino)-5H,6H,7H,8H-pyrido[3,4-d]pyrimidin-7-yl]-8-methyl-1H,2H,3H-pyrido[2,3-b][1,4]oxazine-1-carboxylate: To a stirred solution of tert-butyl 7-bromo-8-methyl-1H,2H,3H-pyrido[2,3-b][1,4]oxazine-1-carboxylate (220.00 mg; 0.65 mmol) and methyl N-[4-({5H,6H,7H,8H-pyrido[3,4-d]pyrimidin-2-yl}amino)phenyl]carbamate (210.00 mg; 0.55 mmol) in 1,4-dioxane (10.00 mL) were added Cs₂CO₃ (646.00 mg; 1.96 mmol) and Pd-PEPPSI-IPentCl 2-methylpyridine (o-picoline) (58.00 mg; 0.07 mmol) in portions at room temperature under N₂ atmosphere. The resulting mixture was stirred for 3 h at 95° C. under N₂ atmosphere. The resulting mixture was concentrated under reduced pressure. The residue was purified by silica gel column and eluted with 18% EtOAc in PE to afford tert-butyl 7-[2-({4-[(methoxycarbonyl)amino]phenyl}amino)-5H,6H,7H,8H-pyrido[3,4-d]pyrimidin-7-yl]-8-methyl-1H,2H,3H-pyrido[2,3-b][1,4]oxazine-1-carboxylate (230.00 mg; 64.2%) as a yellow solid.

methyl N-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}carbamate: To a stirred solution of tert-butyl 7-[2-({4-[(methoxycarbonyl)amino]phenyl}amino)-5H,6H,7H,8H-pyrido[3,4-d]pyrimidin-7-yl]-8-methyl-1H,2H,3H-pyrido[2,3-b][1,4]oxazine-1-carboxylate (200.00 mg; 0.37 mmol) in ethyl acetate (8.00 mL) was added HCl (4M) in EtOAc (8.00 mL; 32.00 mmol) in portions at room temperature under N₂ atmosphere. The resulting mixture was stirred for 1 h at 25° C. under N2 atmosphere. The resulting mixture was basified to pH 9 with Na₂CO₃ (aq.), extracted with EtOAc (4×100 mL). The combined organic layers were concentrated under reduced pressure. The crude product was purified by Prep-HPLC with the following conditions (Column: YMC-Actus Triart C18 ExRS, 30*150 mm, 5 μm; Mobile Phase A: water (10 mmol/L NH₄HCO₃+0.05% NH₄OH), Mobile Phase B: ACN; Flow rate: 60 mL/min.; Gradient: 20% B to 50% B in 8 min., 50% B; Wave Length: 254 nm; RT1 (min.): 7) to afford methyl N-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}carbamate (14.00 mg; 8.3%) as a white solid.

HPLC: 96.7% purity, RT=7.66 min. MS: m/z=448.15 [M+H]⁺. ¹H NMR (400 MHz, DMSO-d6) 9.43-9.38 (m, 1H), 9.35 (s, 1H), 8.29 (s, 1H), 7.63 (d, J=8.5 Hz, 2H), 7.35-7.26 (m, 3H), 5.50 (s, 1H), 4.19 (t, J=4.3 Hz, 2H), 3.94 (s, 2H), 3.64 (s, 3H), 3.31 (s, 2H), 3.12 (d, J=5.9 Hz, 2H), 2.78 (d, J=5.7 Hz, 2H), 2.04 (s, 3H).

### Example 72: Synthesis of Compound 72 N-(7-{8-methyl-1H,2H,3H-pyrido[2,3-b]oxazin-7-yl}-5H,6H,7H,8H-pyrido[3,4-d]pyrimidin-2-yl)-2-(4-methylpiperazin-1-yl)pyridin-4-amine

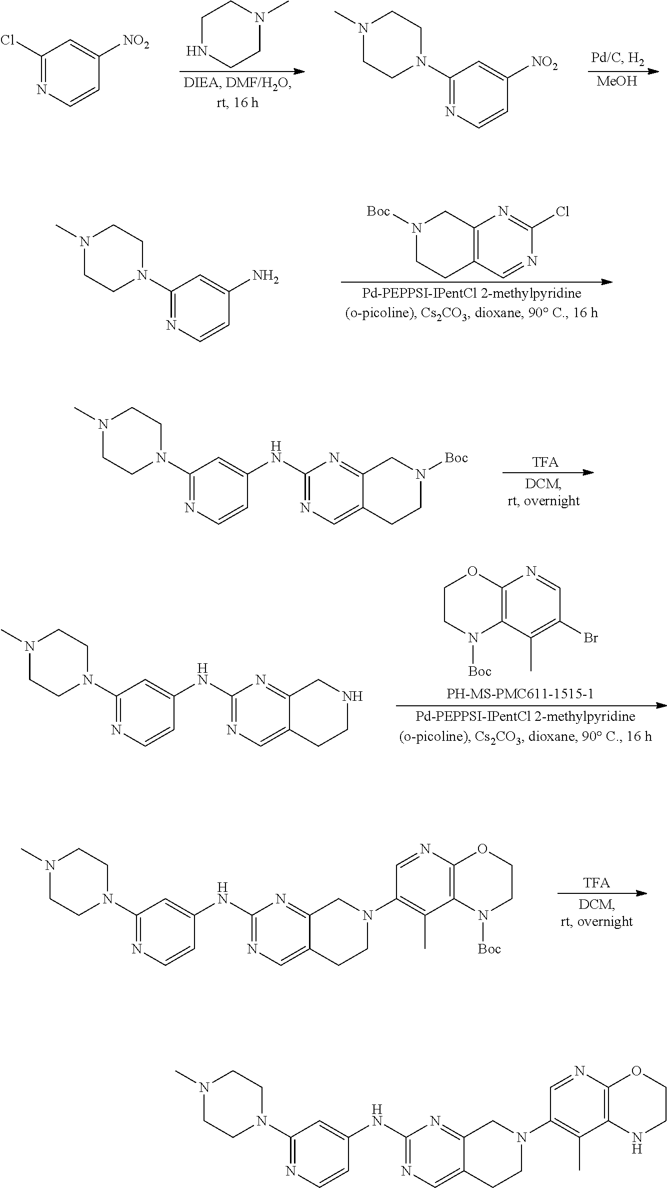

1-methyl-4-(4-nitropyridin-2-yl)piperazine: To a stirred solution of 2-chloro-4-nitropyridine (5.00 g; 29.96 mmol) and 1-methylpiperazine (3.95 g; 37.45 mmol) in DMF (50.00 mL) and water (50.00 mL) was added DIEA (8151.78 mg; 59.92 mmol). The resulting mixture was stirred for overnight at room temperature under nitrogen atmosphere. The residue was purified by silica gel column with the following conditions (DCM/MeOH 19:1) to afford 1-methyl-4-(4-nitropyridin-2-yl) piperazine (1.10 g; 16.24%) as a yellow solid.

2-(4-methylpiperazin-1-yl) pyridin-4-amine: To a solution of 1-methyl-4-(4-nitropyridin-2-yl) piperazine (1.00 g; 4.33 mmol) in MeOH (30.00 mL) was added Palladium on activated carbon (460.31 mg; 0.43 mmol). The mixture was hydrogenated at room temperature for 2 h under hydrogen atmosphere using a hydrogen balloon. The resulting mixture was filtered and the filter cake was washed with 30 mL of EA. The filtrate was concentrated under reduced pressure. The filtrate was concentrated under reduced pressure to afford 2-(4-methylpiperazin-1-yl) pyridin-4-amine (880.00 mg; crude product) as a red solid.

tert-butyl 2-{[2-(4-methylpiperazin-1-yl)pyridin-4-yl]amino}-5H,6H,7H,8H-pyrido[3,4-d]pyrimidine-7-carboxylate: To a solution of tert-butyl 2-chloro-5H,6H,7H,8H-pyrido[3,4-d]pyrimidine-7-carboxylate (400.00 mg; 1.48 mmol) and 2-(4-methylpiperazin-1-yl) pyridin-4-amine (340.00 mg; 1.48 mmol) in 1,4-dioxane (15.00 mL) were added Cs₂CO₃ (1017.00 mg; 2.97 mmol), and Pd-PEPPSI-IPentCl 2-methylpyridine (o-picoline) (131.00 mg; 0.15 mmol). The resulting mixture was stirred for 3 h at 100° C. under nitrogen atmosphere. The resulting mixture was concentrated under reduced pressure. The residue was purified by silica gel column chromatography and eluted with PE:EA (1:2) to afford tert-butyl 2-{[2-(4-methylpiperazin-1-yl)pyridin-4-yl]amino}-5H,6H,7H,8H-pyrido[3,4-d]pyrimidine-7-carboxylate (471.00 mg; 56.43%) as a yellow solid.

2-(4-methylpiperazin-1-yl)-N-{5H,6H,7H,8H-pyrido[3,4-d]pyrimidin-2-yl}pyridin-4-amine: To a stirred solution of tert-butyl 2-{[2-(4-methylpiperazin-1-yl)pyridin-4-yl]amino}-5H,6H,7H,8H-pyrido[3,4-d]pyrimidine-7-carboxylate (400.00 mg; 0.71 mmol) in DCM (15.00 mL) was added TFA (1.00 mL) in portions at room temperature. The mixture was basified to pH [7] with saturated NaHCO₃ (aq.). The resulting mixture was extracted with EtOAc. The combined organic layers were dried over anhydrous Na₂SO₄. After filtration, the filtrate was concentrated under reduced pressure to afford 2-(4-methylpiperazin-1-yl)-N-{5H,6H,7H,8H-pyrido[3,4-d]pyrimidin-2-yl}pyridin-4-amine (248.00 mg; crude product) as a yellow solid.

tert-butyl 8-methyl-7-(2-{[2-(4-methylpiperazin-1-yl)pyridin-4-yl]amino}-5H,6H,7H,8H-pyrido[3,4-d]pyrimidin-7-yl)-1H,2H,3H-pyrido[2,3-b][1,4]oxazine-1-carboxylate: To a solution of 2-(4-methylpiperazin-1-yl)-N-{5H,6H,7H,8H-pyrido[3,4-d]pyrimidin-2-yl}pyridin-4-amine (190.00 mg; 0.57 mmol) and tert-butyl 7-bromo-8-methyl-1H,2H,3H-pyrido[2,3-b][1,4]oxazine-1-carboxylate (199.00 mg; 0.57 mmol) in 1,4-dioxane (15.00 mL) were added Cs₂CO₃ (388.00 mg; 1.13 mmol) and Pd-PEPPSI-IPentCl 2-methylpyridine (o-picoline) (50.00 mg; 0.06 mmol). The resulting mixture was stirred for 3 h at 100° C. under nitrogen atmosphere. The resulting mixture was concentrated under reduced pressure. The residue was purified by silica gel column chromatography and eluted with DCM:MEOH (10:1) to afford tert-butyl 8-methyl-7-(2-{[2-(4-methylpiperazin-1-yl)pyridin-4-yl]amino}-5H,6H,7H,8H-pyrido[3,4-d]pyrimidin-7-yl)-1H,2H,3H-pyrido[2,3-b][1,4]oxazine-1-carboxylate (100.00 mg; 28.74%) as a yellow solid.

N-(7-{8-methyl-1H,2H,3H-pyrido[2,3-b][1,4]oxazin-7-yl}-5H,6H,7H,8H-pyrido[3,4-d]pyrimidin-2-yl)-2-(4-methylpiperazin-1-yl)pyridin-4-amine: To a stirred solution of tert-butyl 8-methyl-7-(2-{[2-(4-methylpiperazin-1-yl)pyridin-4-yl]amino}-5H,6H,7H,8H-pyrido[3,4-d]pyrimidin-7-yl)-1H,2H,3H-pyrido [2,3-b][1,4]oxazine-1-carboxylate (90.00 mg; 0.15 mmol) in DCM (1.50 mL) was added TFA (1.00 mL) in portions at room temperature. The mixture was basified to pH [7] with saturated NaHCO₃ (aq.). The resulting mixture was extracted with EtOAc. The combined organic layers were dried over anhydrous Na₂SO₄. After filtration, the filtrate was concentrated under reduced pressure. The residue was purified by Prep-HPLC with the following conditions (Column: XBridge Shield RP18 OBD Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 30% to 60% in 9 min.; Wave Length: 254 nm; RTT (min.): 7) to afford N-(7-{8-methyl-1H,2H,3H-pyrido[2,3-b][1,4]oxazin-7-yl}-5H,6H,7H,8H-pyrido[3,4-d]pyrimidin-2-yl)-2-(4-methylpiperazin-1-yl)pyridin-4-amine (26.80 mg; 38.60%) as a yellow solid.

HPLC: 99.8% purity, RT=1.93 min. MS: m/z=474.25 [M+H]⁺. ¹H NMR (300 MHz, DMSO-d6) 9.72 (s, 1H), 8.41 (s, 1H), 7.89 (d, J=5.6 Hz, 1H), 7.30 (d, J=6.3 Hz, 2H), 7.07 (dd, J=5.7, 1.6 Hz, 1H), 5.53 (s, 1H), 4.19 (t, J=4.2 Hz, 2H), 3.99 (s, 2H), 3.40 (t, J=5.1 Hz, 4H), 3.30 (s, 2H), 3.13 (t, J=5.6 Hz, 2H), 2.82 (d, J=5.7 Hz, 2H), 2.39 (t, J=5.0 Hz, 4H), 2.20 (s, 3H), 2.04 (s, 3H).

### Example 73: Synthesis of Compound 73 N-(5-((7-(8-methyl-2,3-dihydro-1H-pyrido[2,3-b][1,4]oxazin-7-yl)-5,6,7,8-tetrahydropyrido[3,4-d]pyrimidin-2-yl)amino)pyridin-2-yl)acetamide

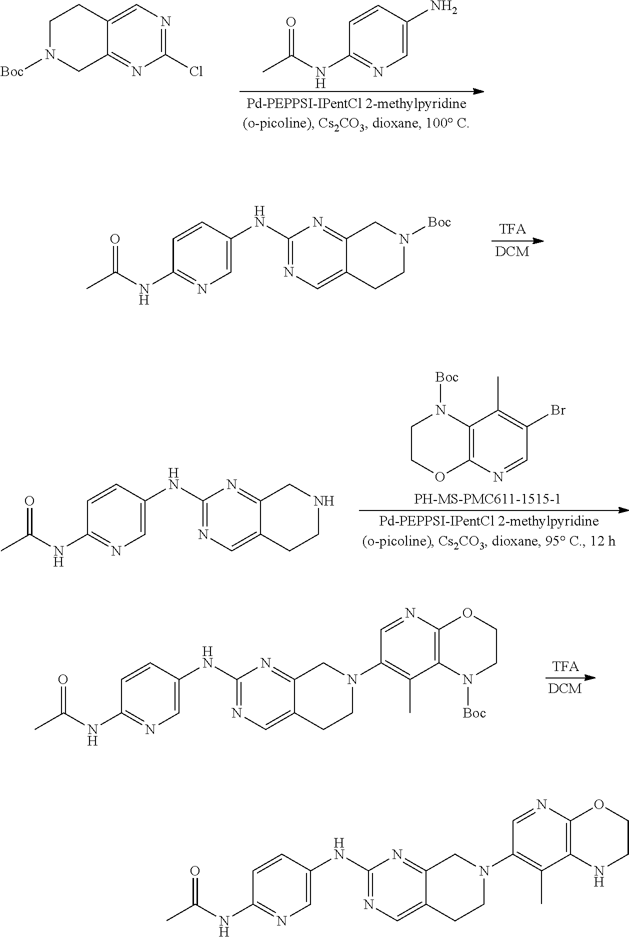

tert-butyl 2-[(6-acetamidopyridin-3-yl)amino]-5H,6H,7H,8H-pyrido[3,4-d]pyrimidine-7-carboxylate: To a stirred solution of tert-butyl 2-chloro-5H,6H,7H,8H-pyrido[3,4-d]pyrimidine-7-carboxylate (450 mg; 1.64 mmol) and N-(5-aminopyridin-2-yl)acetamide (287 mg; 1.80 mmol) in 1,4-dioxane (15 mL) were added Pd-PEPPSI-IPentCl 2-methylpyridine (o-picoline) (145 mg; 0.16 mmol) and Cs₂CO₃ (1122 mg; 3.27 mmol) in portions at room temperature under N₂ atmosphere. The residue was purified by silica gel column chromatography and eluted with PE:EA=3:7 to afford tert-butyl 2-[(6-acetamidopyridin-3-yl)amino]-5H,6H,7H,8H-pyrido[3,4-d]pyrimidine-7-carboxylate as yellow solid (400 mg; 62.3%).

N-[5-({5H,6H,7H,8H-pyrido[3,4-d]pyrimidin-2-yl}amino)pyridin-2-yl]acetamide: To a stirred mixture of tert-butyl 2-[(6-acetamidopyridin-3-yl)amino]-5H,6H,7H,8H-pyrido[3,4-d]pyrimidine-7-carboxylate (380 mg; 0.97 mmol) in DCM (20 mL) was added TFA (4 mL) dropwise at 25 degrees C. The resulting mixture was stirred for 2 h at 25 degrees C. under nitrogen atmosphere. The resulting mixture was concentrated under vacuum. The mixture was basified to pH=8 with NaHCO₃ (aq.). The resulting mixture was extracted with EA (3×30 mL). The combined organic layers were washed with brine and dried over anhydrous MgSO₄. After filtration, the filtrate was concentrated under reduced pressure to afford N-[5-({5H,6H,7H,8H-pyrido[3,4-d]pyrimidin-2-yl}amino)pyridin-2-yl]acetamide as yellow solid (300 mg; 96.7%).

tert-butyl 7-{2-[(6-acetamidopyridin-3-yl)amino]-5H,6H,7H,8H-pyrido[3,4-d]pyrimidin-7-yl}-8-methyl-1H,2H,3H-pyrido[2,3-b][1,4]oxazine-1-carboxylate: To a stirred mixture of N-[5-({5H,6H,7H,8H-pyrido[3,4-d]pyrimidin-2-yl}amino)pyridin-2-yl]acetamide (266 mg; 0.83 mmol) and tert-butyl 7-bromo-8-methyl-1H,2H,3H-pyrido[2,3-b][1,4]oxazine-1-carboxylate (200 mg; 0.59 mmol) in 1,4-dioxane (15 mL) were added Pd-PEPPSI-IPentCl 2-methylpyridine (o-picoline) (50 mg; 0.06 mmol) and Cs₂CO₃ (407 mg; 1.19 mmol) in portions at 25 degrees C. The resulting mixture was stirred for 2 h at 100 degrees C. under nitrogen atmosphere. The resulting mixture was concentrated under vacuum. The residue was purified by silica gel column chromatography and eluted with PE:EA=2:8 to afford tert-butyl 7-{2-[(6-acetamidopyridin-3-yl)amino]-5H,6H,7H,8H-pyrido[3,4-d]pyrimidin-7-yl}-8-methyl-1H,2H,3H-pyrido[2,3-b][1,4]oxazine-1-carboxylate as yellow solid (150 mg; 47.4%).

N-{5-[(7-{8-methyl-1H,2H,3H-pyrido[2,3-b][1,4]oxazin-7-yl}-5H,6H,7H,8H-pyrido[3,4-d]pyrimidin-2-yl)amino]pyridin-2-yl}acetamide: To a stirred mixture of tert-butyl 7-{2-[(6-acetamidopyridin-3-yl)amino]-5H,6H,7H,8H-pyrido[3,4-d]pyrimidin-7-yl}-8-methyl-1H,2H,3H-pyrido[2,3-b][1,4]oxazine-1-carboxylate (130 mg; 0.24 mmol) in DCM (8 mL) was added TFA (2 mL) dropwise at 25 degrees C. The resulting mixture was stirred for 2 h at 25 degrees C. under nitrogen atmosphere. The resulting mixture was concentrated under vacuum. The crude product was purified by Prep-HPLC with the following conditions (Column: Xselect CSH C18 OBD Column 30*150 mm, 5 μm, n; Mobile Phase A: water (0.1% FA), Mobile Phase B: ACN; Flow rate: 60 mL/min.; Gradient: 5% B to 33% B in 8 min., 33% B; Wave Length: 254/220 nm; RT1 (min.): 6.8; Number Of Runs: 1) to afford N-{5-[(7-{8-methyl-1H,2H,3H-pyrido[2,3-b][1,4]oxazin-7-yl}-5H,6H,7H,8H-pyrido[3,4-d]pyrimidin-2-yl)amino]pyridin-2-yl}acetamide as yellow solid (25.1 mg; 23.7%).

HPLC 99.59% purity, 2.38 min. MS: m/z=433.20 [M+H]⁺. ¹H NMR (300 MHz, DMSO-d6): 10.28 (s, 1H), 9.58 (s, 1H), 8.60 (d, J=2.7 Hz, 1H), 8.34 (s, 1H), 8.20-8.08 (m, 1H), 7.97 (d, J=9.0 Hz, 1H), 7.29 (s, 1H), 5.51 (s, 1H), 4.19 (t, J=4.3 Hz, 2H), 3.96 (s, 2H), 3.30 (s, 2H), 3.12 (t, J=5.6 Hz, 2H), 2.79 (s, 2H), 2.05 (d, J=5.9 Hz, 6H).

### Example 74: Synthesis of Compound 74 2-fluoro-N-methyl-4-((7-(8-methyl-2,3-dihydro-1H-pyrido[2,3-b][1,4]oxazin-7-yl)-5,6,7,8-tetrahydropyrido[3,4-d]pyrimidin-2-yl)amino)benzamide

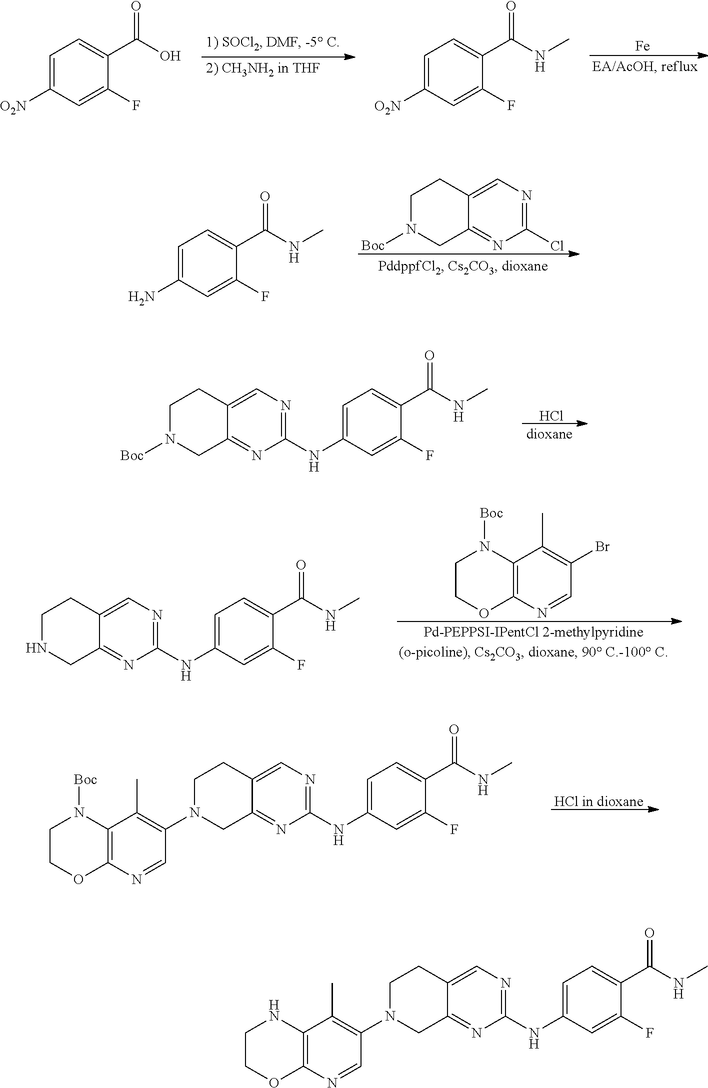

2-fluoro-N-methyl-4-nitrobenzamide: To a stirred solution of 2-fluoro-4-nitrobenzoic acid (2 g; 10.48 mmol) in DMF (10 mL) was added SOCL2 (0.91 mL; 11.98 mmol) dropwise at −5 degree C. under N₂ atmosphere. The resulting mixture was stirred for 1 h at 0 degree C. under N₂ atmosphere. Then was added CH₃NH₂ in THF (40 mL; 80.00 mmol) in portions at 0 degree C. under N₂ atmosphere. The resulting mixture was stirred for overnight at room temperature under N₂ atmosphere. The resulting mixture was diluted with water (50 mL). The pH value of the solution was adjusted to 7-8 with NaHCO₃, extracted with EtOAc (3×50 mL). The combined organic layers were washed with brine (50 mL) and dried over anhydrous Na₂SO₄. The resulting mixture was concentrated under reduced pressure to afford 2-fluoro-N-methyl-4-nitrobenzamide as yellow solid (840 mg; 39.7%).

4-amino-2-fluoro-N-methylbenzamide: To a stirred solution of 2-fluoro-N-methyl-4-nitrobenzamide (0.81 g; 4.01 mmol) and Fe (1.15 g; 19.51 mmol) in EtOH (15 mL) and H₂O (10 mL) was added NH₄Cl (0.88 g; 15.63 mmol) in portions at room temperature under N₂ atmosphere. The resulting mixture was stirred for 1 h at 80 degree C. under N₂ atmosphere. The residue was purified by silica gel column and eluted with 75% EtOAc in PE to afford 4-amino-2-fluoro-N-methylbenzamide as yellow solid (0.61 g; 90.2%).

3 tert-butyl 2-{[3-fluoro-4-(methylcarbamoyl)phenyl]amino}-5H,6H,7H,8H-pyrido[3,4-d]pyrimidine-7-carboxylate: To a stirred solution of tert-butyl 2-chloro-5H,6H,7H,8H-pyrido[3,4-d]pyrimidine-7-carboxylate (800 mg; 2.92 mmol) and 4-amino-2-fluoro-N-methylbenzamide (600 mg; 3.56 mmol) in 1,4-dioxane (15 mL) were added PddppfCl₂ (250 mg; 0.32 mmol) and Cs₂CO₃ (1.94 g; 5.8 mmol) in portions at room temperature under N₂ atmosphere. The resulting mixture was stirred for 2 h at 100 degree C. under N₂ atmosphere. The resulting mixture was concentrated under reduced pressure. The residue was purified by silica gel column and eluted with 75% EtOAc in PE to afford tert-butyl 2-{[3-fluoro-4-(methylcarbamoyl)phenyl]amino}-5H,6H,7H,8H-pyrido[3,4-d]pyrimidine-7-carboxylate as yellow orange solid (460 mg, 44.9%).

2-fluoro-N-methyl-4-({5H,6H,7H,8H-pyrido[3,4-d]pyrimidin-2-yl}amino)benzamide: To a stirred solution of tert-butyl 2-{[3-fluoro-4-(methylcarbamoyl)phenyl]amino}-5H,6H,7H,8H-pyrido[3,4-d]pyrimidine-7-carboxylate (460 mg; 0.51 mmol) in 1,4-dioxane (10 mL) was added 4 M HCl (gas) in 1,4-dioxane (5 mL) in portions at room temperature under N₂ atmosphere. The resulting mixture was stirred for 30 min. at room temperature under N₂ The resulting mixture was concentrated under reduced pressure to afford 2-fluoro-N-methyl-4-({5H,6H,7H,8H-pyrido[3,4-d]pyrimidin-2-yl}amino)benzamide as yellow solid (300 mg; 86.9%).

tert-butyl 7-(2-{[3-fluoro-4-(methylcarbamoyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate: To a stirred solution of 2-fluoro-N-methyl-4-({5H,6H,7H,8H-pyrido[3,4-d]pyrimidin-2-yl}amino)benzamide (300 mg; 0.83 mmol) in 1,4-dioxane (6.00 mL) was added Cs₂CO₃ (1.13 g; 3.29 mmol) in portions at room temperature under N₂ atmosphere. The resulting mixture was stirred for 10 min. at room temperature. Then were added tert-butyl 7-bromo-8-methyl-1H,2H,3H-pyrido[2,3-b][1,4]oxazine-1-carboxylate (425.00 mg; 1.16 mmol) and Pd-PEPPSI-IPentCl 2-methylpyridine (o-picoline) (83.70 mg; 0.09 mmol) in portions at room temperature under N₂ atmosphere. The resulting mixture was stirred for 16 h at 100 degree C. under N₂ atmosphere. The resulting mixture was diluted with water (50 mL), extracted with EtOAc (3×50 mL). The resulting mixture was concentrated under reduced pressure. The residue was purified by silica gel column and eluted with 75% EtOAc in PE to afford tert-butyl 7-(2-{[3-fluoro-4-(methylcarbamoyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate as yellow orange solid (521 mg; 81.1%).

2-fluoro-N-methyl-4-[(7-{8-methyl-1H,2H,3H-pyrido[2,3-b][1,4]oxazin-7-yl}-5H,6H,7H,8H-pyrido[3,4-d]pyrimidin-2-yl)amino]benzamide: To a stirred solution of tert-butyl 7-(2-{[3-fluoro-4-(methylcarbamoyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (500 mg; 0.65 mmol) in 1,4-dioxane was added 4 M HCl (gas) in 1,4-dioxane (5 mL) in portions at room temperature under N₂ atmosphere. The resulting mixture was stirred for 1 h at room temperature under N₂ atmosphere. The resulting mixture was diluted with water (50 mL), adjusted PH=7-8 with NaHCO₃ (aq.), and extracted with EtOAc (3×50 mL). The combined organic layers were washed with brine (50 mL) and dried over anhydrous Na₂SO₄. The resulting mixture was concentrated under reduced pressure. The residue was purified by Perp-HPLC with the following condition: Column: XBridge Prep OBD C18 Column, 30*150 mm, 5 μm; Mobile Phase A: water (10 mmol/L NH4HCO3+0.05% NH₄OH), Mobile Phase B: ACN; Flow rate: 60 mL/min.; Gradient: 20% B to 50% B in 9 min., 50% B; Wave Length: 254 nm; RT1 (min.): 7; Number Of Runs: 3, to afford 2-fluoro-N-methyl-4-[(7-{8-methyl-1H,2H,3H-pyrido[2,3-b][1,4]oxazin-7-yl}-5H,6H,7H,8H-pyrido[3,4-d]pyrimidin-2-yl)amino]benzamide as yellow solid (40.50 mg)

HPLC 96.11% purity, 3.061 min. MS: m/z=450.15 [M+H]⁺. ¹H NMR (300 MHz, DMSO-d6): 10.05 (s, 1H), 8.43 (s, 1H), 7.96-7.82 (m, 2H), 7.60 (t, J=8.6 Hz, 1H), 7.51 (dd, J=8.6, 2.0 Hz, 1H), 7.30 (s, 1H), 5.53 (s, 1H), 4.19 (t, J=4.3 Hz, 2H), 4.01 (s, 2H), 3.34 (s, 2H), 3.13 (t, J=5.5 Hz, 2H), 2.80 (dd, J=19.5, 5.1 Hz, 5H), 2.04 (s, 3).

### Example 75: Synthesis of Compound 75 7-(8-methyl-2,3-dihydro-1H-pyrido[2,3-b][1,4]oxazin-7-yl)-N-(2-(methylsulfonyl)pyridin-4-yl)-5,6,7,8-tetrahydropyrido[3,4-d]pyrimidin-2-amine

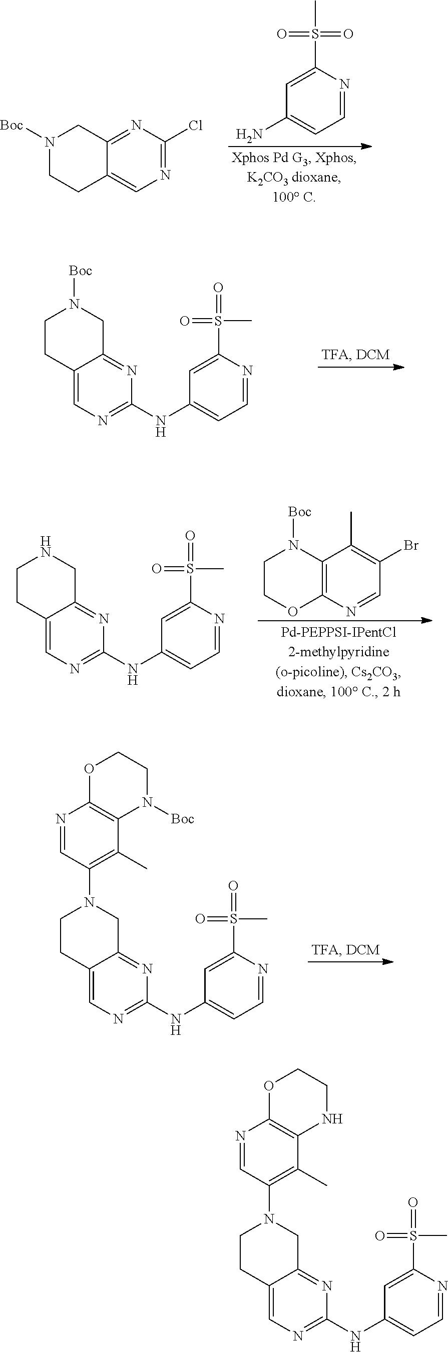

tert-butyl 2-[(2-methanesulfonylpyridin-4-yl)amino]-5H,6H,7H,8H-pyrido[3,4-d]pyrimidine-7-carboxylate: A solution of tert-butyl 2-chloro-5H,6H,7H,8H-pyrido[3,4-d]pyrimidine-7-carboxylate (300 mg; 1.11 mmol), 2-methanesulfonylpyridin-4-amine (201.61 mg; 1.11 mmol), Xphos Pd G3 (99.10 mg; 0.11 mmol), Xphos (55.81 mg; 0.11 mmol) and K₂CO₃ (323.62 mg; 2.22 mmol) in 1,4-dioxane (20 mL) was stirred for 2 h at 100 degrees C. under N₂ atmosphere. The solvent was removed under vacuum and the residue was purified by silica gel column chromatography and eluted with PE/EtOAc (5:1) to afford tert-butyl 2-[(2-methanesulfonylpyridin-4-yl)amino]-5H,6H,7H,8H-pyrido[3,4-d]pyrimidine-7-carboxylate as off-white solid (370 mg; 82.0%).

2-methanesulfonyl-N-{5H,6H,7H,8H-pyrido[3,4-d]pyrimidin-2-yl}pyridin-4-amine: A solution of tert-butyl 2-[(2-methanesulfonylpyridin-4-yl)amino]-5H,6H,7H,8H-pyrido[3,4-d]pyrimidine-7-carboxylate (360 mg; 0.89 mmol) and TFA (0.60 mL) in DCM (3 mL) was stirred for 3 h at room temperature. The solvent was removed under vacuum and the residue was purified by reverse flash chromatography with the following conditions: column, C18 silica gel; mobile phase, H2O/ACN. The resulting mixture was concentrated under vacuum to afford 2-methanesulfonyl-N-{5H,6H,7H,8H-pyrido[3,4-d]pyrimidin-2-yl}pyridin-4-amine as white solid (270 mg; 99.6%).

tert-butyl 7-{2-[(2-methanesulfonylpyridin-4-yl)amino]-5H,6H,7H,8H-pyrido[3,4-d]pyrimidin-7-yl}-8-methyl-1H,2H,3H-pyrido[2,3-b][1,4]oxazine-1-carboxylate: A solution of 2-methanesulfonyl-N-{5H,6H,7H,8H-pyrido[3,4-d]pyrimidin-2-yl}pyridin-4-amine (270 mg; 1.11 mmol), tert-butyl 7-bromo-8-methyl-1H,2H,3H-pyrido[2,3-b][1,4]oxazine-1-carboxylate (470.00 mg; 1.34 mmol), CS₂CO₃ (800.00 mg; 2.21 mmol) and Pd-PEPPSI-IPentCl 2-methylpyridine (o-picoline) (100 mg; 0.11 mmol) in 1,4-dioxane (10 mL) was stirred for 2 h at 100 degrees C. under N₂ atmosphere. The solvent was removed under vacuum and the residue was purified by silica gel column chromatography and eluted with PE/EtOAc (4:1) to afford tert-butyl 7-{2-[(2-methanesulfonylpyridin-4-yl)amino]-5H,6H,7H,8H-pyrido[3,4-d]pyrimidin-7-yl}-8-methyl-1H,2H,3H-pyrido[2,3-b][1,4]oxazine-1-carboxylate as white solid (230 mg; 47%).

2-methanesulfonyl-N-(7-{8-methyl-1H,2H,3H-pyrido[2,3-b][1,4]oxazin-7-yl}-5H,6H,7H,8H-pyrido[3,4-d]pyrimidin-2-yl)pyridin-4-amine: A solution of tert-butyl 7-{2-[(2-methanesulfonylpyridin-4-yl)amino]-5H,6H,7H,8H-pyrido[3,4-d]pyrimidin-7-yl}-8-methyl-1H,2H,3H-pyrido[2,3-b][1,4]oxazine-1-carboxylate (220 mg; 0.40 mmol) and TFA (0.50 mL) in DCM (2 mL) was stirred for 1 h at room temperature. The resulting mixture was concentrated under vacuum. The crude product was purified by Prep-HPLC with the following conditions: Column: XBridge Shield RP18 OBD Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 19% B to 47% B in 9 min., 47% B; Wave Length: 254 nm; RT1 (min.): 7. This resulted in 2-methanesulfonyl-N-(7-{8-methyl-1H,2H,3H-pyrido[2,3-b][1,4]oxazin-7-yl}-5H,6H,7H,8H-pyrido[3,4-d]pyrimidin-2-yl)pyridin-4-amine as white solid (81.20 mg; 44.8%).

HPLC 99.37% purity, 2.84 min. MS: m/z=454.05 [M+H]⁺. ¹H NMR (300 MHz, DMSO-d6): 10.58 (s, 1H), 8.54-8.43 (m, 3H), 8.00 (dd, J=5.6, 2.2 Hz, 1H), 7.28 (s, 1H), 5.54 (s, 1H), 4.17 (t, J=4.3 Hz, 2H), 4.01 (s, 2H), 3.28 (s, 2H), 3.22 (s, 3H), 3.12 (d, J=5.8 Hz, 2H), 2.85 (s, 2H), 2.02 (s, 3H).

### Example 76: Synthesis of Compound 76 7-(8-methyl-2,3-dihydro-1H-pyrido[2,3-b][1,4]oxazin-7-yl)-N-(2-(tetrahydro-2H-pyran-4-yl)pyridin-4-yl)-5,6,7,8-tetrahydropyrido[3,4-d]pyrimidin-2-amine

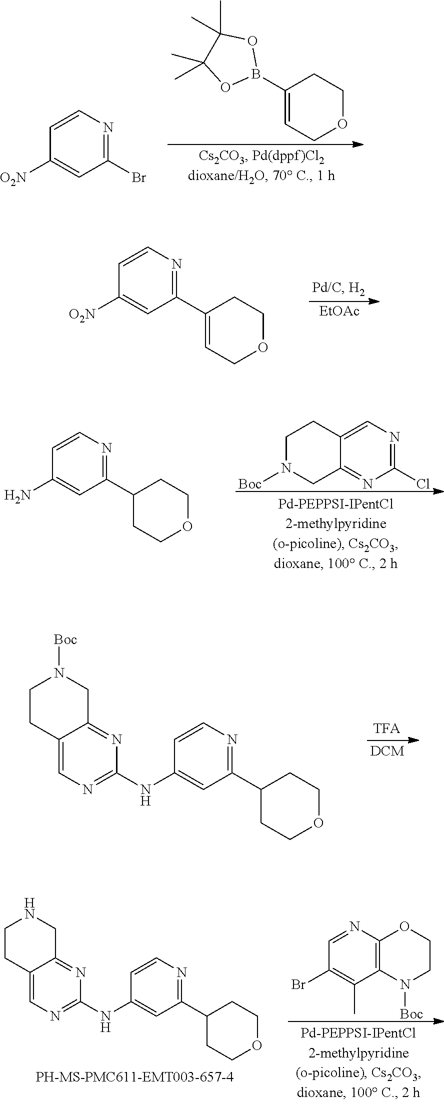

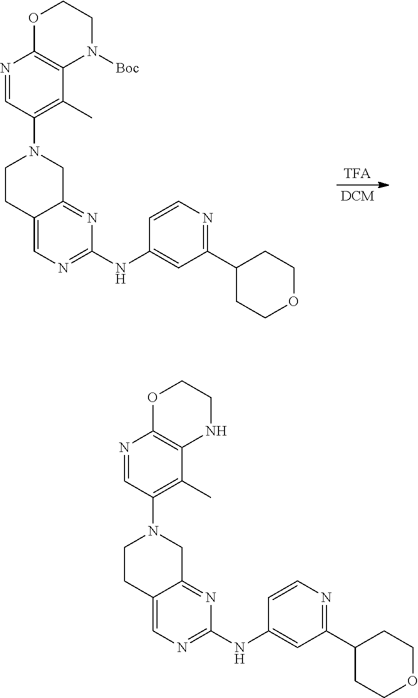 [SMILES: Cc1c(N2CCc3cnc(Nc4ccnc(C5CCOCC5)c4)nc3C2)cnc2c1N(C(=O)OC(C)(C)C)CCO2 ; Cc1c(N2CCc3cnc(Nc4ccnc(C5CCOCC5)c4)nc3C2)cnc2c1NCCO2]

2-(3,6-dihydro-2H-pyran-4-yl)-4-nitropyridine: To a stirred mixture of 2-bromo-4-nitropyridine (2 g; 9.36 mmol) and 2-(3,6-dihydro-2H-pyran-4-yl)-4,4,5,5-tetramethyl-1,3,2-dioxaborolane (2.48 g; 11.23 mmol) in 1,4-dioxane (20 mL) were added Pd(dppf)Cl₂ (0.76 g; 0.94 mmol), H₂O (4 mL) and Cs₂CO₃ (6.42 g; 18.72 mmol) in portions at 25 degrees C. The resulting mixture was stirred for 2 h at 100 degrees C. under nitrogen atmosphere. The resulting mixture was concentrated under vacuum. The residue was purified by silica gel column chromatography and eluted with PE:EA=1:1 to afford 2-(3,6-dihydro-2H-pyran-4-yl)-4-nitropyridine as yellow solid (1.60 g; 82.9%).

2-(oxan-4-yl)pyridin-4-amine: To a stirred mixture of 2-(3,6-dihydro-2H-pyran-4-yl)-4-nitropyridine (800 mg; 3.88 mmol) in ethyl acetate (15 mL) was added Pd/C (300 mg; 0.28 mmol) in portions at 25 degrees C. under nitrogen atmosphere. The resulting mixture was stirred for overnight at 25 degrees C. under hydrogen (gas) atmosphere. After completed reaction, the resulting mixture was filtered and the filter cake was washed with EA (3×30 mL). The filtrate was concentrated under reduced pressure to afford 2-(oxan-4-yl)pyridin-4-amine as yellow solid (600 mg, 82.4%).

tert-butyl 2-{[2-(oxan-4-yl)pyridin-4-yl]amino}-5H,6H,7H,8H-pyrido[3,4-d]pyrimidine-7-carboxylate: To a stirred mixture of 2-(oxan-4-yl)pyridin-4-amine (251.00 mg; 1.34 mmol) and tert-butyl 2-chloro-5H,6H,7H,8H-pyrido[3,4-d]pyrimidine-7-carboxylate (300.00 mg; 1.11 mmol) in 1,4-dioxane (15 mL) were added Pd-PEPPSI-IPentCl 2-methylpyridine (o-picoline) (99 mg; 0.11 mmol) and Cs₂CO₃ (732 mg; 2.22 mmol) in portions at 25 degrees C. The resulting mixture was stirred for 2 h at 100 degrees C. under nitrogen atmosphere. The resulting mixture was concentrated under vacuum. The residue was purified by silica gel column chromatography and eluted with PE:EA=4:6 to afford tert-butyl 2-{[2-(oxan-4-yl)pyridin-4-yl]amino}-5H,6H,7H,8H-pyrido[3,4-d]pyrimidine-7-carboxylate as yellow solid (250 mg; 49.1%).

2-(oxan-4-yl)-N-{5H,6H,7H,8H-pyrido[3,4-d]pyrimidin-2-yl}pyridin-4-amine: To a stirred mixture of tert-butyl 2-{[2-(oxan-4-yl)pyridin-4-yl]amino}-5H,6H,7H,8H-pyrido[3,4-d]pyrimidine-7-carboxylate (230 mg; 0.50 mmol) in DCM (8 mL) was added TFA (2 mL) dropwise at 25 degrees C. The resulting mixture was stirred for 2 h at 25 degrees C. under nitrogen atmosphere. The resulting mixture was concentrated under vacuum. The reaction was diluted with H₂O at 25 degrees C. The mixture was neutralized to pH=8 with NaHCO₃ (aq.). The resulting mixture was extracted with EA (3×50 mL). The combined organic layers were washed with brine and dried over anhydrous MgSO₄. After filtration, the filtrate was concentrated under reduced pressure to afford 2-(oxan-4-yl)-N-{5H,6H,7H,8H-pyrido[3,4-d]pyrimidin-2-yl}pyridin-4-amine as yellow solid (170 mg; 77.2%).

tert-butyl 8-methyl-7-(2-{[2-(oxan-4-yl)pyridin-4-yl]amino}-5H,6H,7H,8H-pyrido[3,4-d]pyrimidin-7-yl)-1H,2H,3H-pyrido[2,3-b][1,4]oxazine-1-carboxylate: To a stirred mixture of 2-(oxan-4-yl)-N-{5H,6H,7H,8H-pyrido[3,4-d]pyrimidin-2-yl}pyridin-4-amine (159.2 mg; 0.36 mmol) and tert-butyl 7-bromo-8-methyl-1H,2H,3H-pyrido[2,3-b][1,4]oxazine-1-carboxylate (120 mg; 0.36 mmol) in 1,4-dioxane (10 mL) were added Pd-PEPPSI-IPentCl 2-methylpyridine (o-picoline) (32 mg; 0.04 mmol) and Cs₂CO₃ (239 mg; 0.73 mmol) in portions at 25 degrees C. The resulting mixture was stirred for 2 h at 100 degrees C. under nitrogen atmosphere. The resulting mixture was concentrated under vacuum. The residue was purified by silica gel column chromatography and eluted with EA to afford tert-butyl 8-methyl-7-(2-{[2-(oxan-4-yl)pyridin-4-yl]amino}-5H,6H,7H,8H-pyrido[3,4-d]pyrimidin-7-yl)-1H,2H,3H-pyrido[2,3-b][1,4]oxazine-1-carboxylate as yellow solid (150 mg; 61.6%).

N-(7-{8-methyl-1H,2H,3H-pyrido[2,3-b][1,4]oxazin-7-yl}-5H,6H,7H,8H-pyrido[3,4-d]pyrimidin-2-yl)-2-(oxan-4-yl)pyridin-4-amine: To a stirred mixture of tert-butyl 8-methyl-7-(2-{[2-(oxan-4-yl)pyridin-4-yl]amino}-5H,6H,7H,8H-pyrido[3,4-d]pyrimidin-7-yl)-1H,2H,3H-pyrido[2,3-b][1,4]oxazine-1-carboxylate (120 mg; 0.18 mmol) in DCM (8 mL) was added TFA (2 mL) dropwise at 25 degrees C. The resulting mixture was stirred for 2 h at 25 degrees C. under nitrogen atmosphere. The resulting mixture was concentrated under vacuum. The crude product was purified by Prep-HPLC with the following conditions (Column: XBridge Prep OBD C18 Column, 30*150 mm, 5 μm; Mobile Phase A: water (10 mmol/L NH4HCO3+0.05% NH₄OH), Mobile Phase B: ACN; Flow rate: 60 m/min.; Gradient: 19% B to 46% B in 9 min., 46% B; Wave Length: 254 nm; RT1 (min.): 7; Number Of Runs: 2) to afford N-(7-{8-methyl-1H,2H,3H-pyrido[2,3-b][1,4]oxazin-7-yl}-5H,6H,7H,8H-pyrido[3,4-d]pyrimidin-2-yl)-2-(oxan-4-yl)pyridin-4-amine as off-white solid (21.7 mg).

HPLC 99.58% purity, 2.44 min. MS: m/z=460.15 [M+H]⁺. ¹H NMR (400 MHz, DMSO-d6): 9.96 (s, 1H), 8.44 (s, 1H), 8.25 (d, J=5.6 Hz, 1H), 7.68-7.58 (m, 2H), 7.30 (s, 1H), 5.54 (t, J=2.8 Hz, 1H), 4.19 (t, J=4.3 Hz, 2H), 4.01 (s, 2H), 3.94 (dt, J=11.1, 3.3 Hz, 2H), 3.49-3.38 (m, 2H), 3.33-3.27 (m, 2H), 3.14 (t, J=5.6 Hz, 2H), 2.81 (dt, J=12.5, 6.4 Hz, 3H), 2.04 (s, 3H), 1.74 (dt, J=9.1, 4.3 Hz, 4H).

### Example 77: Synthesis of Compound 77 7-(8-methyl-2,3-dihydro-1H-pyrido[2,3-b][1,4]oxazin-7-yl)-N-(2-morpholinopyridin-4-yl)-5,6,7,8-tetrahydropyrido[3,4-d]pyrimidin-2-amine

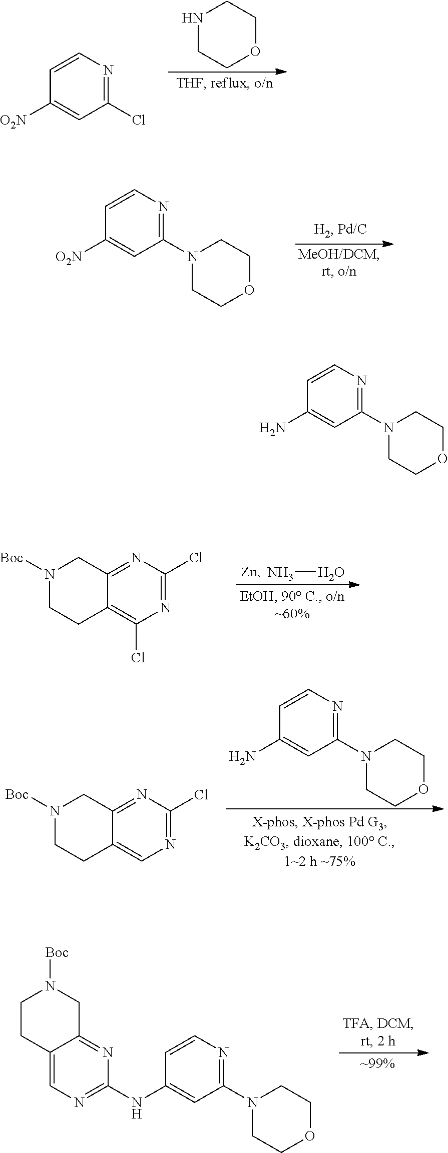

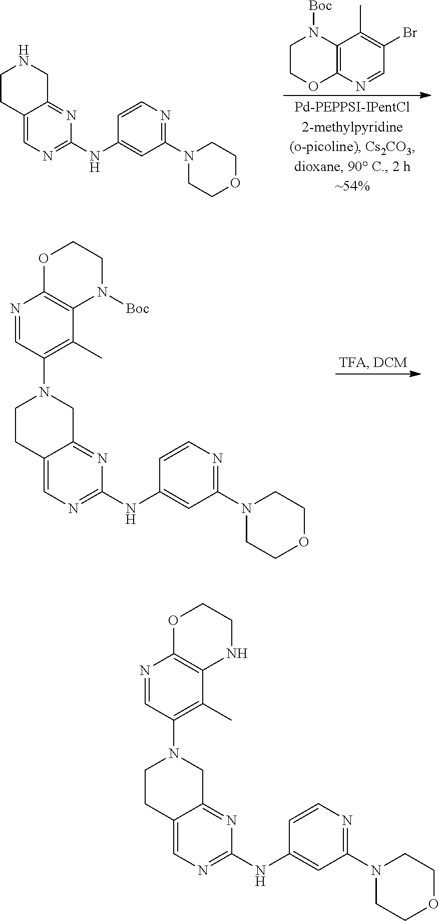 [SMILES: c1cc(Nc2ncc3c(n2)CNCC3)cc(N2CCOCC2)n1 ; Cc1c(Br)cnc2c1N(C(=O)OC(C)(C)C)CCO2 ; Cc1c(N2CCc3cnc(Nc4ccnc(N5CCOCC5)c4)nc3C2)cnc2c1N(C(=O)OC(C)(C)C)CCO2 ; Cc1c(N2CCc3cnc(Nc4ccnc(N5CCOCC5)c4)nc3C2)cnc2c1NCCO2]

4-(4-nitropyridin-2-yl)morpholine: To a stirred solution of 2-chloro-4-nitropyridine (90 g; 539.29 mmol) and morpholine (148.37 mL; 1617.87 mmol) in THF (900 mL) was added morpholine (148.37 mL; 1617.87 mmol) dropwise at room temperature. The resulting mixture was stirred for overnight at 70 degrees C. under nitrogen atmosphere. The resulting mixture was allowed to cool down to room temperature. The residue was purified by silica gel column chromatography and eluted with PE:EtOAc (95:5) to afford 4-(4-nitropyridin-2-yl)morpholine as orange yellow solid (45 g; 39.5%).

2-(morpholin-4-yl)pyridin-4-amine: To a stirred solution of 4-(4-nitropyridin-2-yl)morpholine (44 g; 208.45 mmol) in MeOH (600 mL) and DCM (200 mL) was added Pd/C (20 g; 18.79 mmol) in portions at room temperature. The resulting mixture was stirred for overnight at room temperature under nitrogen atmosphere. The resulting mixture was filtered and the filter cake was washed with MeOH (3×500 mL). The filtrate was concentrated under reduced pressure. The residue was purified by silica gel column chromatography and eluted with DCM:MeOH (20:1) to afford 2-(morpholin-4-yl)pyridin-4-amine as off white solid (40 g; 66.3%).

tert-butyl 2-{[2-(morpholin-4-yl)pyridin-4-yl]amino}-5H,6H,7H,8H-pyrido[3,4-d]pyrimidine-7-carboxylate: To a stirred mixture of tert-butyl 2-chloro-5H,6H,7H,8H-pyrido[3,4-d]pyrimidine-7-carboxylate (34 g; 126.05 mmol) and 2-(morpholin-4-yl)pyridin-4-amine (36.46 g; 126.05 mmol) in 1,4-dioxane (400 mL) was added K₂CO₃ (36.60 g; 252.11 mmol) and Xphos Pd G3 (5.61 g; 6.30 mmol) and Xphos (6.68 g; 12.61 mmol) in portions at room temperature. The resulting mixture was stirred for 1 h at 100 degrees C. under nitrogen atmosphere. The resulting mixture was allowed to cool down to room temperature. The resulting mixture was diluted with water. The resulting mixture was extracted with EtOAc (3×500 mL). The combined organic layers were washed with brine and dried over anhydrous Na₂SO₄. After filtration, the filtrate was concentrated under reduced pressure. The residue was purified by silica gel column chromatography and eluted with 50% EA in PE to afford tert-butyl 2-{[2-(morpholin-4-yl)pyridin-4-yl]amino}-5H,6H,7H,8H-pyrido[3,4-d]pyrimidine-7-carboxylate as off-white solid (40 g; 75.5%).

2-(morpholin-4-yl)-N-{5H,6H,7H,8H-pyrido[3,4-d]pyrimidin-2-yl}pyridin-4-amine: To a stirred mixture of tert-butyl 2-{[2-(morpholin-4-yl)pyridin-4-yl]amino}-5H,6H,7H,8H-pyrido[3,4-d]pyrimidine-7-carboxylate (38.9 g; 92.50 mmol) in DCM (300 mL) was added TFA (100 mL) dropwise at 0 degrees C. The resulting mixture was stirred for 2 h at room temperature under nitrogen atmosphere. The mixture was neutralized to pH 7 with NaHCO₃. The mixture was extracted with DCM (3×500 mL). The combined organic layers were washed with brine and dried over anhydrous Na₂SO₄. After filtration, the filtrate was concentrated under reduced pressure to afford 2-(morpholin-4-yl)-N-{5H,6H,7H,8H-pyrido[3,4-d]pyrimidin-2-yl}pyridin-4-amine as off-white solid (29 g; 99.7%).

tert-butyl 8-methyl-7-(2-{[2-(morpholin-4-yl)pyridin-4-yl]amino}-5H,6H,7H,8H-pyrido[3,4-d]pyrimidin-7-yl)-1H,2H,3H-pyrido[2,3-b][1,4]oxazine-1-carboxylate: To a stirred mixture of 2-(morpholin-4-yl)-N-{5H,6H,7H,8H-pyrido[3,4-d]pyrimidin-2-yl}pyridin-4-amine (19 g; 60.44 mmol) and tert-butyl 7-bromo-8-methyl-1H,2H,3H-pyrido[2,3-b][1,4]oxazine-1-carboxylate (24.46 g; 72.52 mmol) in 1,4-dioxane (200 mL) was added Cs₂CO₃ (41.46 g; 120.87 mmol) and Pd-PEPPSI-IPentCl 2-methylpyridine (o-picoline) (2.67 g; 3.02 mmol) in portions at room temperature. The resulting mixture was stirred for overnight at 110 degrees C. under nitrogen atmosphere. The resulting mixture was allowed to cool down to room temperature. The resulting mixture was diluted with water. The resulting mixture was extracted with EtOAc (3×500 mL). The combined organic layers were washed with brine and dried over anhydrous Na₂SO₄. After filtration, the filtrate was concentrated under reduced pressure. The residue was purified by silica gel column chromatography and eluted with DCM:MeOH (100:3) to afford the crude. The crude was purified by reverse flash chromatography with the following conditions: column, C18 silica gel; mobile phase, ACN in water, 70% to 80% gradient in 20 min.; detector, UV 254 nm, to afford tert-butyl 8-methyl-7-(2-{[2-(morpholin-4-yl)pyridin-4-yl]amino}-5H,6H,7H,8H-pyrido[3,4-d]pyrimidin-7-yl)-1H,2H,3H-pyrido[2,3-b][1,4]oxazine-1-carboxylate as off-white solid (20 g; 58.3%).

N-(7-{8-methyl-1H,2H,3H-pyrido[2,3-b][1,4]oxazin-7-yl}-5H,6H,7H,8H-pyrido[3,4-d]pyrimidin-2-yl)-2-(morpholin-4-yl)pyridin-4-amine: To a stirred mixture of tert-butyl 8-methyl-7-(2-{[2-(morpholin-4-yl)pyridin-4-yl]amino}-5H,6H,7H,8H-pyrido[3,4-d]pyrimidin-7-yl)-1H,2H,3H-pyrido[2,3-b][1,4]oxazine-1-carboxylate (19.8 g; 34.92 mmol) in DCM (300 mL) was added TFA (100 mL) dropwise at 0 degrees C. The resulting mixture was stirred for 1 h at room temperature under nitrogen atmosphere. The resulting mixture was concentrated under vacuum. The mixture was basified to pH 7-8 with saturated NaHCO₃ solution. The precipitated solids were collected by filtration and washed with water (3×50 mL). This resulted in N-(7-{8-methyl-1H,2H,3H-pyrido[2,3-b][1,4]oxazin-7-yl}-5H,6H,7H,8H-pyrido[3,4-d]pyrimidin-2-yl)-2-(morpholin-4-yl)pyridin-4-amine as off-white solid (12.56 g; 76.9%).

HPLC 98.48% purity, 2.42 min. MS: m/z=461.30[M+H]⁺. ¹H NMR (400 MHz, DMSO-d6): 9.91 (s, 1H), 8.43 (s, 1H), 7.92 (d, J=5.9 Hz, 1H), 7.39 (s, 1H), 7.29 (s, 1H), 7.12 (dd, J=5.9, 1.7 Hz, 1H), 5.54 (s, 1H), 4.19 (t, J=4.3 Hz, 2H), 4.01 (s, 2H), 3.74-3.67 (m, 4H), 3.38 (d, J=9.7 Hz, 4H), 3.29 (d, J=5.3 Hz, 2H), 3.13 (t, J=5.7 Hz, 2H), 2.82 (t, J=5.5 Hz, 2H), 2.04 (s, 3H).

### Example 78: Synthesis of Compound 78 (5-((7-(5-(methoxymethyl)pyridin-3-yl)-5,6,7,8-tetrahydropyrido[3,4-d]pyrimidin-2-yl)amino)-2-(tetrahydro-2H-pyran-4-yl)phenyl)methanol

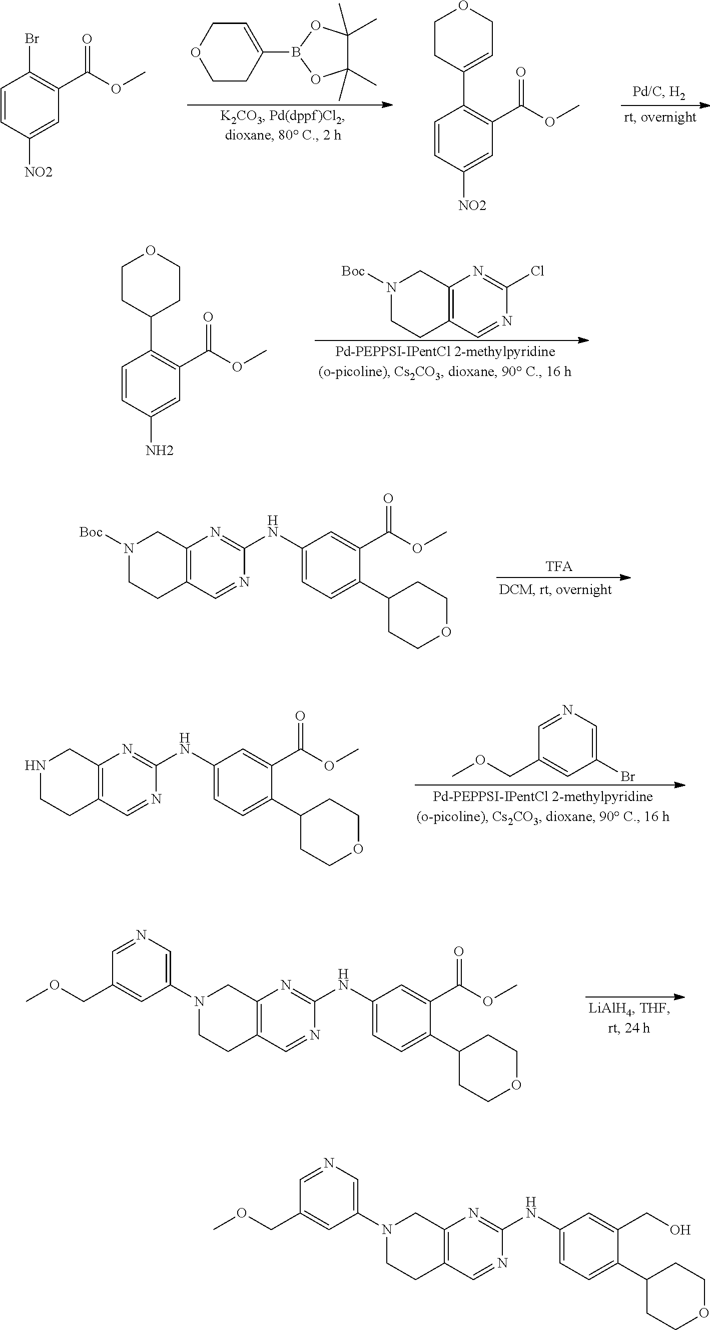

methyl 2-(3,6-dihydro-2H-pyran-4-yl)-5-nitrobenzoate: To a solution of methyl 2-bromo-5-nitrobenzoate (5 g; 19.23 mmol) and 2-(3,6-dihydro-2H-pyran-4-yl)-4,4,5,5-tetramethyl-1,3,2-dioxaborolane (8079 mg; 38.46 mmol) in 1,4-dioxane (20 mL) and H2O (5 mL) were added potassium carbonate (5677 mg; 38.46 mmol) and Pd(dppf)Cl₂·CH₂Cl₂ (1619 mg; 1.92 mmol). The resulting mixture was stirred for 3 h at 90 degrees C. under nitrogen atmosphere. The resulting mixture was concentrated under reduced pressure. The residue was purified by silica gel column chromatography and eluted with PE/EA (4:1) to afford methyl 2-(3,6-dihydro-2H-pyran-4-yl)-5-nitrobenzoate as white solid (4.4 g; 86.8%).

methyl 5-amino-2-(oxan-4-yl)benzoate: To a solution of methyl 2-(3,6-dihydro-2H-pyran-4-yl)-5-nitrobenzoate (1.5 g; 5.70 mmol) in AcOEt (20.00 mL) was added Palladium/Carbon (750 mg; 0.70 mmol) in a pressure tank. The mixture was hydrogenated at room temperature under 30 psi of hydrogen pressure for overnight. The resulting mixture was filtered and the filter cake was washed with 300 mL of EA. The filtrate was concentrated under reduced pressure. The filtrate was concentrated under reduced pressure to afford methyl 5-amino-2-(oxan-4-yl)benzoate as white solid (1.1 g; 80.7%).

tert-butyl 2-{[3-(methoxycarbonyl)-4-(oxan-4-yl)phenyl]amino}-5H,6H,7H,8H-pyrido[3,4-d]pyrimidine-7-carboxylate: To a solution of methyl 5-amino-2-(oxan-4-yl)benzoate (1.05 g; 4.46 mmol) and tert-butyl 2-chloro-5H,6H,7H,8H-pyrido[3,4-d]pyrimidine-7-carboxylate (1204.00 mg) in 1,4-dioxane (20.00 mL) were added Cs₂CO₃ (3062 mg) and Pd-PEPPSI-IPentCl 2-methylpyridine (o-picoline) (395 mg). The resulting mixture was stirred for overnight at 90 degrees C. under nitrogen atmosphere. The resulting mixture was concentrated under vacuum. The residue was purified by silica gel column chromatography and eluted with PE/EtOAc (2:3) to afford tert-butyl 2-{[3-(methoxycarbonyl)-4-(oxan-4-yl)phenyl]amino}-5H,6H,7H,8H-pyrido[3,4-d]pyrimidine-7-carboxylate yellow solid (1 g; 47.4%).

methyl 2-(oxan-4-yl)-5-({5H,6H,7H,8H-pyrido[3,4-d]pyrimidin-2-yl}amino)benzoate: To a stirred solution of tert-butyl 2-{[3-(methoxycarbonyl)-4-(oxan-4-yl)phenyl]amino}-5H,6H,7H,8H-pyrido[3,4-d]pyrimidine-7-carboxylate (900 mg; 1.92 mmol) in DCM (20 mL) was added TFA (1096 mg) in portions at room temperature. Then the resulting mixture was stirred for additional 3 h at room temperature. The mixture was basified to pH [7] with saturated NaHCO₃ (aq.). The resulting mixture was extracted with EtOAc. The combined organic layers were washed with NaCl (aq.) and dried over anhydrous Na₂SO₄. After filtration, the filtrate was concentrated under reduced pressure to afford methyl 2-(oxan-4-yl)-5-({5H,6H,7H,8H-pyrido[3,4-d]pyrimidin-2-yl}amino)benzoate as yellow solid (650 mg; 88.9%).

methyl 5-({7-[5-(methoxymethyl)pyridin-3-yl]-5H,6H,7H,8H-pyrido[3,4-d]pyrimidin-2-yl}amino)-2-(oxan-4-yl)benzoate: To a solution of methyl 2-(oxan-4-yl)-5-({5H,6H,7H,8H-pyrido[3,4-d]pyrimidin-2-yl}amino)benzoate (640 mg; 1.74 mmol) and 3-bromo-5-(methoxymethyl)pyridine (702 mg; 3.47 mmol) in 1,4-dioxane (2 mL) were added Cs₂CO₃ (1788 mg; 5.21 mmol) and Pd-PEPPSI-IPentCl 2-methylpyridine (o-picoline) (154 mg; 0.17 mmol). The resulting mixture was stirred for overnight at 90 degrees C. under nitrogen atmosphere. The resulting mixture was concentrated under reduced pressure. The residue was purified by silica gel column chromatography and eluted with DCM/MEOH (20:1) to afford methyl 5-({7-[5-(methoxymethyl)pyridin-3-yl]-5H,6H,7H,8H-pyrido[3,4-d]pyrimidin-2-yl}amino)-2-(oxan-4-yl)benzoate as yellow solid (600 mg; 51.4%).

[5-({7-[5-(methoxymethyl)pyridin-3-yl]-5H,6H,7H,8H-pyrido[3,4-d]pyrimidin-2-yl}amino)-2-(oxan-4-yl)phenyl]methanol hydrochloride: To a solution of methyl 5-({7-[5-(methoxymethyl)pyridin-3-yl]-5H,6H,7H,8H-pyrido[3,4-d]pyrimidin-2-yl}amino)-2-(oxan-4-yl)benzoate (300 mg; 0.45 mmol) in THF (15 mL) were added LiAlH4 (143 mg; 3.58 mmol). The resulting mixture was stirred for overnight at room temperature under nitrogen atmosphere. The mixture was basified to pH [7] with saturated NaHCO₃ (aq.). The resulting mixture was extracted with EtOAc. The combined organic layers were washed with NaCl (aq.) and dried over anhydrous Na₂SO₄. After filtration, the filtrate was concentrated under reduced pressure. The residue was purified by Prep-HPLC with the following conditions (Column: XBridge Prep OBD C18 Column, 30*150 mm, 5 μm; Mobile Phase A: water (10 mmol/L NH4HCO3+0.05% NH₄OH), Mobile Phase B: ACN; Flow rate: 60 mL/min.; Gradient: 20% B to 50% B in 9 min., 50% B; Wave Length: 254 nm; RT1 (min.): 7; Number Of Runs: 6) to afford[5-({7-[5-(methoxymethyl)pyridin-3-yl]-5H,6H,7H,8H-pyrido[3,4-d]pyrimidin-2-yl}amino)-2-(oxan-4-yl)phenyl]methanol hydrochloride as yellow solid (13.90 mg).

HPLC 92.61% purity, 2.93 min. MS: m/z=462.00 [M+H]⁺. ¹H NMR (300 MHz, Methanol-d4): 8.45 (d, J=2.7 Hz, 1H), 8.30 (s, 1H), 8.13 (d, J=3.9 Hz, 2H), 7.57-7.45 (m, 2H), 7.42 (d, J=8.3 Hz, 1H), 4.75 (s, 2H), 4.70 (s, 2H), 4.64 (s, 2H), 4.06 (dd, J=11.2, 3.6 Hz, 2H), 3.87 (t, J=5.7 Hz, 2H), 3.61 (td, J=11.7, 2.2 Hz, 2H), 3.50 (s, 3H), 3.15 (ddt, J=11.6, 7.5, 3.7 Hz, 1H), 3.02 (t, J=5.8 Hz, 2H), 1.97-1.80 (m, 2H), 1.72 (d, J=12.5 Hz, 2H).

### Example 79: Synthesis of Compound 79 7-(8-methyl-2,3-dihydro-1H-pyrido[2,3-b][1,4]oxazin-7-yl)-N-(3-(methylsulfonyl)phenyl)-5,6,7,8-tetrahydropyrido[3,4-d]pyrimidin-2-amine

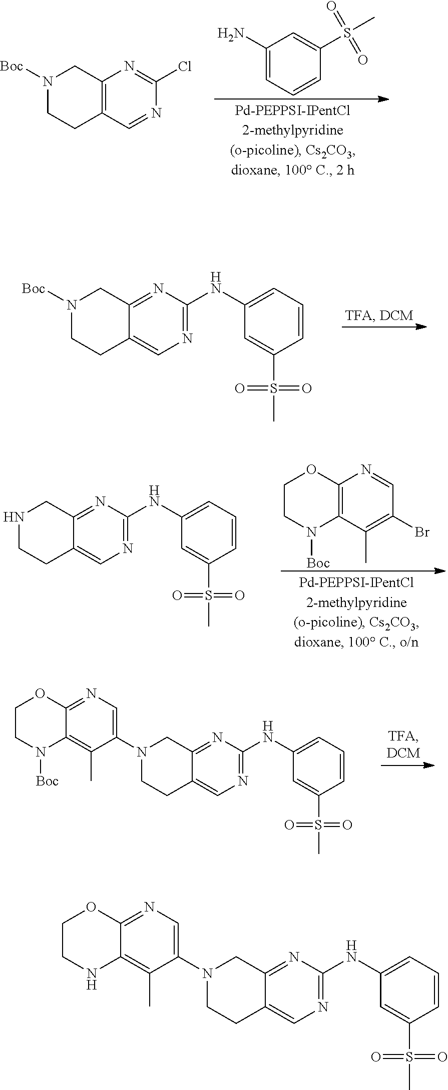

tert-butyl 2-[(3-methanesulfonylphenyl)amino]-5H,6H,7H,8H-pyrido[3,4-d]pyrimidine-7-carboxylate: To a stirred solution of tert-butyl 2-chloro-5H,6H,7H,8H-pyrido[3,4-d]pyrimidine-7-carboxylate (600 mg; 2.22 mmol) and 3-methanesulfonylaniline (458 mg; 2.54 mmol) in 1,4-dioxane (20 mL) were added Pd-PEPPSI-IPentCl 2-methylpyridine (o-picoline) (188 mg; 0.21 mmol) and Cs₂CO₃ (2.19 g; 6.39 mmol) in portions at room temperature under N₂ atmosphere. The resulting mixture was stirred for 2 h at 100 degree C. under N₂ atmosphere. The resulting mixture was diluted with water (50 mL), extracted with EtOAc (3×50 mL). The combined organic layers were washed with brine (50 mL) and dried over anhydrous Na₂SO₄. The resulting mixture was concentrated under reduced pressure. The residue was purified by silica gel column and eluted with 70% EtOAc in PE to afford tert-butyl 2-[(3-methanesulfonylphenyl)amino]-5H,6H,7H,8H-pyrido[3,4-d]pyrimidine-7-carboxylate as yellow solid (920 mg; 89.8%).

N-(3-methanesulfonylphenyl)-5H,6H,7H,8H-pyrido[3,4-d]pyrimidin-2-amine: To a stirred solution of tert-butyl 2-[(3-methanesulfonylphenyl)amino]-5H,6H,7H,8H-pyrido[3,4-d]pyrimidine-7-carboxylate (450 mg; 0.98 mmol) in DCM (6 mL) was added TFA (2 mL) dropwise at 0 degree C. under N₂ atmosphere. The resulting mixture was stirred for 1 h at room temperature under N₂ The residue was diluted with water, then adjusted to pH 6-7 with NaHCO₃. Then extracted with EtOAc (3×50 mL). The combined organic layers were washed with brine (50 mL) and dried over anhydrous Na₂SO₄. The resulting mixture was concentrated under reduced pressure. The crude product was slurry with PE:EA=3:1 to afford N-(3-methanesulfonylphenyl)-5H,6H,7H,8H-pyrido[3,4-d]pyrimidin-2-amine as yellow solid (270 mg; 85.3%).

tert-butyl 7-{2-[(3-methanesulfonylphenyl)amino]-5H,6H,7H,8H-pyrido[3,4-d]pyrimidin-7-yl}-8-methyl-1H,2H,3H-pyrido[2,3-b][1,4]oxazine-1-carboxylate: To a stirred solution of N-(3-methanesulfonylphenyl)-5H,6H,7H,8H-pyrido[3,4-d]pyrimidin-2-amine (250 mg; 0.77 mmol) and tert-butyl 7-bromo-8-methyl-1H,2H,3H-pyrido[2,3-b][1,4]oxazine-1-carboxylate (297 mg; 0.88 mmol) in 1,4-dioxane (5 mL) were added Pd-PEPPSI-IPentCl 2-methylpyridine (o-picoline) (70 mg; 0.08 mmol) and Cs₂CO₃ (805 mg; 2.35 mmol) in portions at room temperature under N₂ atmosphere. The resulting mixture was stirred for overnight at 100 degree C. under N₂ atmosphere. LCMS: Complete conversion. The resulting mixture was diluted with water (50 mL), extracted with EtOAc (3×50 mL). The combined organic layers were washed with brine (50 mL) and dried over anhydrous Na₂SO₄. The resulting mixture was concentrated under reduced pressure. The residue was purified by silica gel column and eluted with 70% EtOAc in PE to afford tert-butyl 7-{2-[(3-methanesulfonylphenyl)amino]-5H,6H,7H,8H-pyrido[3,4-d]pyrimidin-7-yl}-8-methyl-1H,2H,3H-pyrido[2,3-b][1,4]oxazine-1-carboxylate as yellow solid (330 mg; 60.3%).

N-(3-methanesulfonylphenyl)-7-{8-methyl-1H,2H,3H-pyrido[2,3-b][1,4]oxazin-7-yl}-5H,6H,7H,8H-pyrido[3,4-d]pyrimidin-2-amine: To a stirred solution of tert-butyl 7-{2-[(3-methanesulfonylphenyl)amino]-5H,6H,7H,8H-pyrido[3,4-d]pyrimidin-7-yl}-8-methyl-1H,2H,3H-pyrido[2,3-b][1,4]oxazine-1-carboxylate (310 mg; 0.44 mmol) in DCM (3 mL) was added TFA (1 mL) dropwise at 0 degree C. under N₂ atmosphere. The resulting mixture was stirred for 30 min. at room temperature under N₂ atmosphere. The resulting mixture was concentrated under reduced pressure. The residue was purified by Prep-HPLC with the following condition: Column: XBridge Prep OBD C18 Column, 19*250 mm, 5 μm; Mobile Phase A: water (10 mmol/L NH₄HCO₃+0.05% NH₄OH), Mobile Phase B: MeOH—HPLC; Flow rate: 20 mL/min; Gradient: 20% B to 50% B in 8 min., 50% B; Wave Length: 254 nm; RTT (min.): 7.4; Number Of Runs: 4, to afford N-(3-methanesulfonylphenyl)-7-{8-methyl-1H,2H,3H-pyrido[2,3-b][1,4]oxazin-7-yl}-5H,6H,7H,8H-pyrido[3,4-d]pyrimidin-2-amine brown solid (41.20 mg; 19.3%).

HPLC 92.54% purity, 3.15 min. MS: m/z=453.05 [M+H]⁺. ¹H NMR (400 MHz, Methanol-d4): 8.55 (q, J=1.5 Hz, 1H), 8.37 (s, 1H), 7.98-7.91 (m, 1H), 7.59-7.51 (m, 2H), 7.37 (s, 1H), 4.51 (t, J=4.4 Hz, 2H), 4.10 (s, 2H), 3.56 (t, J=4.5 Hz, 2H), 3.27 (t, J=5.6 Hz, 2H), 3.14 (s, 3H), 2.94 (t, J=5.7 Hz, 2H), 2.28 (s, 3H).

### Example 80: Synthesis of Compound 80 1-(2-ethyl-4-((7-(8-methyl-2,3-dihydro-1H-pyrido[2,3-b][1,4]oxazin-7-yl)-5,6,7,8-tetrahydropyrido[3,4-d]pyrimidin-2-yl)amino)phenyl)piperidin-4-ol

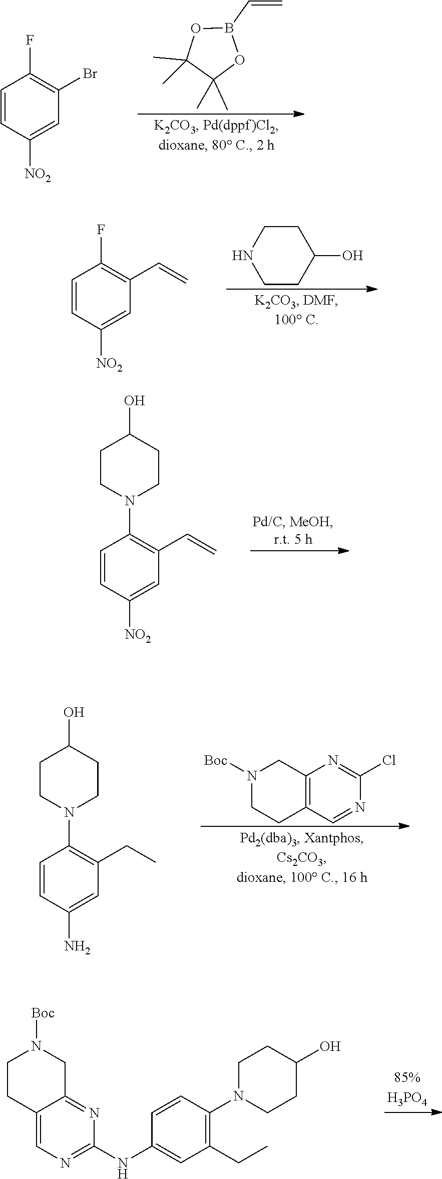

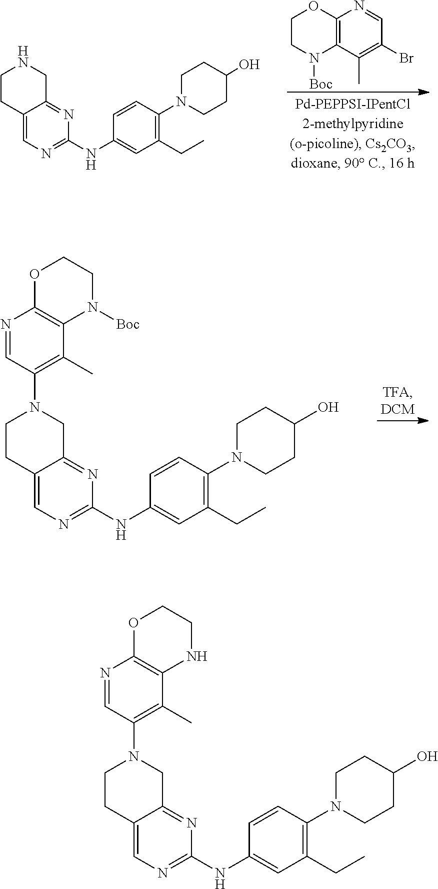 [SMILES: CCc1cc(Nc2ncc3c(n2)CNCC3)ccc1N1CCC(O)CC1 ; Cc1c(Br)cnc2c1N(C(=O)OC(C)(C)C)CCO2 ; CCc1cc(Nc2ncc3c(n2)CN(c2cnc4c(c2C)N(C(=O)OC(C)(C)C)CCO4)CC3)ccc1N1CCC(O)CC1 ; CCc1cc(Nc2ncc3c(n2)CN(c2cnc4c(c2C)NCCO4)CC3)ccc1N1CCC(O)CC1]

2-ethenyl-1-fluoro-4-nitrobenzene: To a solution of 2-bromo-1-fluoro-4-nitrobenzene (7 g; 31.82 mmol) and 2-ethenyl-4,4,5,5-tetramethyl-1,3,2-dioxaborolane (4.9 g; 31.82 mmol) in 1,4-dioxane (70 mL) were added Pd(dppf)Cl₂·CH₂Cl₂ (2736 mg; 3.18 mmol) and Potassium Carbonate (8885 mg; 63.64 mmol). The resulting mixture was stirred for 3 h at 90 degrees C. under nitrogen atmosphere. The resulting mixture was concentrated under reduced pressure. The residue was purified by silica gel column chromatography and eluted with PE/EA (1:1) to afford 2-ethenyl-1-fluoro-4-nitrobenzene as yellow solid (4.4 g; 77.1%).

1-(2-ethenyl-4-nitrophenyl)piperidin-4-ol: A solution of 2-ethenyl-1-fluoro-4-nitrobenzene (4.4 g; 26.33 mmol), piperidin-4-ol (2.66 g; 26.33 mmol) and K₂CO₃ (7.66 g; 52.65 mmol) in DMF (50 mL) was stirred for 3 h at 100 degrees C. under argon atmosphere. The reaction was quenched with H₂O at room temperature. The resulting mixture was extracted with EtOAc. The combined organic layers were washed with NaCl (aq.) and dried over anhydrous Na₂SO₄. After filtration, the filtrate was concentrated under reduced pressure. The residue was purified by silica gel column chromatography and eluted with DCM/MEOH (10:1) to afford 1-(2-ethenyl-4-nitrophenyl)piperidin-4-ol as yellow solid (5.2 g; 77.8%).

1-(4-amino-2-ethylphenyl)piperidin-4-ol: A solution of 1-(2-ethenyl-4-nitrophenyl)piperidin-4-ol (5.1 g; 20.54 mmol) and Palladium/Carbon (2187 mg; 2.06 mmol) in MeOH (60 mL) was stirred for 5 h at 25 degrees under hydrogen atmosphere. The resulting mixture was filtered and the filter cake was washed with 300 mL of MeOH. The filtrate was concentrated under reduced pressure. The residue was purified by silica gel column chromatography and eluted with PE/EtOAc (1:1) to afford 1-(4-amino-2-ethylphenyl)piperidin-4-ol as yellow solid (2.1 g; 44.7%).

tert-butyl 2-{[3-ethyl-4-(4-hydroxypiperidin-1-yl)phenyl]amino}-5H,6H,7H,8H-pyrido[3,4-d]pyrimidine-7-carboxylate: To a solution of tert-butyl 2-chloro-5H,6H,7H,8H-pyrido[3,4-d]pyrimidine-7-carboxylate (1 g; 3.71 mmol) and 1-(4-amino-2-ethylphenyl)piperidin-4-ol (843 mg; 3.71 mmol) in 1,4-dioxane were added Cs₂CO₃ (2544 mg; 7.42 mmol), tris(dibenzylideneacetone)dipalladium (378 mg; 0.37 mmol) and Xantphos (433 mg; 0.74 mmol). The resulting mixture was stirred for 16 h at 100 degrees C. under nitrogen atmosphere. The resulting mixture was concentrated under reduced pressure. The residue was purified by silica gel column chromatography and eluted with DCM/MEOH (9:1) to afford tert-butyl 2-{[3-ethyl-4-(4-hydroxypiperidin-1-yl)phenyl]amino}-5H,6H,7H,8H-pyrido[3,4-d]pyrimidine-7-carboxylate as yellow solid (1.1 g; 58.4%).

1-[2-ethyl-4-({5H,6H,7H,8H-pyrido[3,4-d]pyrimidin-2-yl}amino)phenyl]piperidin-4-ol: A solution of tert-butyl 2-{[3-ethyl-4-(4-hydroxypiperidin-1-yl)phenyl]amino}-5H,6H,7H,8H-pyrido[3,4-d]pyrimidine-7-carboxylate (1100.00 mg; 2.43 mmol) and H₃PO₄ (10.00 mL) was stirred for 3 h at room temperature under argon atmosphere. The mixture was basified to pH [7] with saturated NaHCO₃ (aq.) and extracted by 3×30 mL of EA. The combined organic layers were washed with 3×30 mL brine and dried over anhydrous Na₂SO₄. After filtration, the filtrate was concentrated under reduced pressure. The residue was purified by silica gel column chromatography and eluted with DCM/MEOH (5:1) to afford 1-[2-ethyl-4-({5H,6H,7H,8H-pyrido[3,4-d]pyrimidin-2-yl}amino)phenyl]piperidin-4-ol as yellow solid (570 mg; 66.5%).

tert-butyl 7-(2-{[3-ethyl-4-(4-hydroxypiperidin-1-yl)phenyl]amino}-5H,6H,7H,8H-pyrido[3,4-d]pyrimidin-7-yl)-8-methyl-1H,2H,3H-pyrido[2,3-b][1,4]oxazine-1-carboxylate: To a solution of 1-[2-ethyl-4-({5H,6H,7H,8H-pyrido[3,4-d]pyrimidin-2-yl}amino)phenyl]piperidin-4-ol (565 mg; 1.60 mmol) and tert-butyl 7-bromo-8-methyl-1H,2H,3H-pyrido[2,3-b][1,4]oxazine-1-carboxylate (554.00 mg; 1.60 mmol) in 1,4-dioxane (15.00 mL) were added Cs₂CO₃ (1097 mg; 3.20 mmol) and Pd-PEPPSI-IPentCl 2-methylpyridine (o-picoline) (142 mg; 0.16 mmol). The resulting mixture was stirred for overnight at 90 degrees C. under nitrogen atmosphere. The resulting mixture was concentrated under reduced pressure. The residue was purified by silica gel column chromatography and eluted with DCM/MEOH (6:1) to afford tert-butyl 7-(2-{[3-ethyl-4-(4-hydroxypiperidin-1-yl)phenyl]amino}-5H,6H,7H,8H-pyrido[3,4-d]pyrimidin-7-yl)-8-methyl-1H,2H,3H-pyrido[2,3-b][1,4]oxazine-1-carboxylate as yellow solid (320 mg; 25.6%).

1-{2-ethyl-4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}piperidin-4-ol: A solution of tert-butyl 7-(2-{[3-ethyl-4-(4-hydroxypiperidin-1-yl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (565 mg; 0.94 mmol) and TFA (1 mL) and DCM (4 mL) was stirred for overnight at room temperature under argon atmosphere. The resulting mixture was concentrated under reduced pressure. The crude product was purified by Prep-HPLC with the following conditions (Column: XBridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 26% B to 56% B in 9 min., 56% B; Wave Length: 254 nm; RT1 (min.): 7; Number Of Runs: 2) to afford 1-{2-ethyl-4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}piperidin-4-ol as yellow solid (17.20 mg).

HPLC 99.60% purity, 4.21 min. MS: m/z=502.00 [M+H]+. ¹H NMR (300 MHz, DMSO-d6): 9.26 (s, 1H), 8.28 (s, 1H), 7.63-7.43 (m, 2H), 7.27 (s, 1H), 6.98 (d, J=9.4 Hz, 1H), 5.52 (s, 1H), 4.62 (d, J=4.3 Hz, 1H), 4.19 (s, 2H), 3.92 (s, 2H), 3.56 (s, 1H), 3.10 (d, J=5.7 Hz, 2H), 2.88 (d, J=11.6 Hz, 2H), 2.77 (s, 2H), 2.58 (d, J=7.8 Hz, 6H), 2.03 (s, 3H), 1.83 (d, J=11.9 Hz, 2H), 1.55 (t, J=9.5 Hz, 2H), 1.17 (t, J=7.5 Hz, 3H).

### Example 81: Synthesis of Compound 81 5-((7-(8-methyl-2,3-dihydro-1H-pyrido[2,3-b][1,4]oxazin-7-yl)-5,6,7,8-tetrahydropyrido[3,4-d]pyrimidin-2-yl)amino)-1,3-dihydrobenzo[c]thiophene 2,2-dioxide

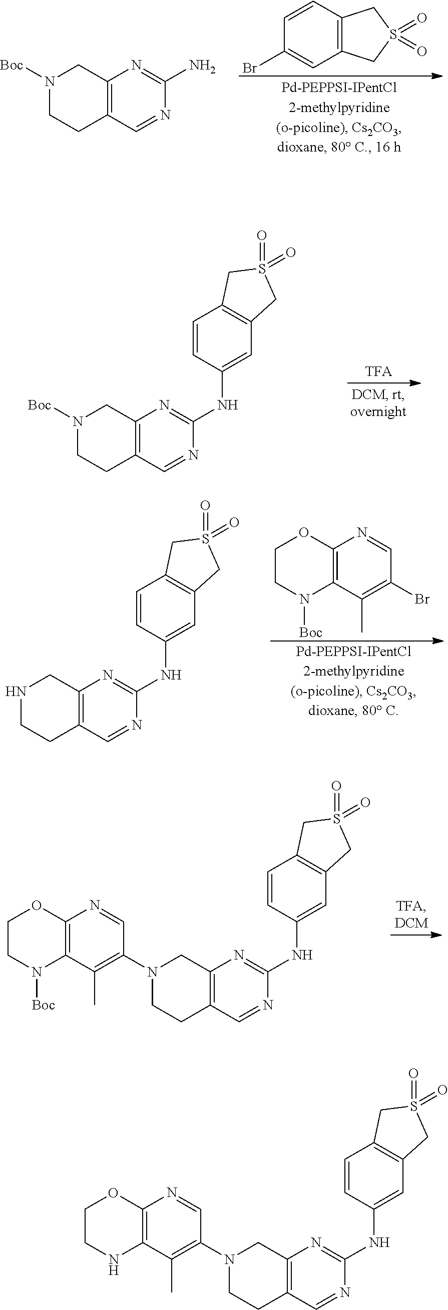

tert-butyl 2-[(2,2-dioxo-1,3-dihydro-2?6-benzothiophen-5-yl)amino]-5H,6H,7H,8H-pyrido[3,4-d]pyrimidine-7-carboxylate: To a solution of tert-butyl 2-amino-5H,6H,7H,8H-pyrido[3,4-d]pyrimidine-7-carboxylate (200 mg; 0.58 mmol) and 5-bromo-1,3-dihydro-2?6-benzothiophene-2,2-dione (150 mg; 0.58 mmol) in 1,4-dioxane (10 mL) were added Cs₂CO₃ (395 mg; 1.15 mmol) and Pd-PEPPSI-IPentCl 2-methylpyridine (o-picoline) (51 mg; 0.06 mmol). The resulting mixture was stirred for 3 h at 90 degrees C. under nitrogen atmosphere. The resulting mixture was concentrated under reduced pressure. The residue was purified by silica gel column chromatography and eluted with PE/EA (1:1) to afford tert-butyl 2-[(2,2-dioxo-1,3-dihydro-2?6-benzothiophen-5-yl)amino]-5H,6H,7H,8H-pyrido[3,4-d]pyrimidine-7-carboxylate as yellow solid (150 mg; 60.3%).

5-({5H,6H,7H,8H-pyrido[3,4-d]pyrimidin-2-yl}amino)-1,3-dihydro-2?6-benzothiophene-2,2-dione: To a stirred solution of tert-butyl 2-[(2,2-dioxo-1,3-dihydro-2?6-benzothiophen-5-yl)amino]-5H,6H,7H,8H-pyrido[3,4-d]pyrimidine-7-carboxylate (140 mg; 0.32 mmol) in DCM (5 mL) was added TFA (2 mL) in portions at room temperature. Then the resulting mixture was stirred for overnight at room temperature. The mixture was basified to pH [7] with saturated NaHCO₃ (aq.). The resulting mixture was extracted with EtOAc. The combined organic layers were washed with NaCl (aq.) and dried over anhydrous Na₂SO₄. After filtration, the filtrate was concentrated under reduced pressure to afford 5-({5H,6H,7H,8H-pyrido[3,4-d]pyrimidin-2-yl}amino)-1,3-dihydro-2?6-benzothiophene-2,2-dione as yellow solid (105 mg; 99.5%).

tert-butyl 7-{2-[(2,2-dioxo-1,3-dihydro-2?6-benzothiophen-5-yl)amino]-5H,6H,7H,8H-pyrido[3,4-d]pyrimidin-7-yl}-8-methyl-1H,2H,3H-pyrido[2,3-b][1,4]oxazine-1-carboxylate: To a solution of 5-({5H,6H,7H,8H-pyrido[3,4-d]pyrimidin-2-yl}amino)-1,3-dihydro-2?6-benzothiophene-2,2-dione (95 mg; 0.29 mmol) and tert-butyl 7-bromo-8-methyl-1H,2H,3H-pyrido[2,3-b][1,4]oxazine-1-carboxylate (99 mg; 0.29 mmol) in 1,4-dioxane (10 mL) were added Pd-PEPPSI-IPentCl 2-methylpyridine (o-picoline) (26 mg; 0.03 mmol) and Cs₂CO₃ (200 mg; 0.58 mmol). The resulting mixture was stirred for 3 h at 80 degrees C. under nitrogen atmosphere. The resulting mixture was concentrated under vacuum. The residue was purified by silica gel column chromatography and eluted with PE/EtOAc (1:9) to afford tert-butyl 7-{2-[(2,2-dioxo-1,3-dihydro-2?6-benzothiophen-5-yl)amino]-5H,6H,7H,8H-pyrido[3,4-d]pyrimidin-7-yl}-8-methyl-1H,2H,3H-pyrido[2,3-b][1,4]oxazine-1-carboxylate as yellow solid (75 mg; 40.0%).

5-[(7-{8-methyl-1H,2H,3H-pyrido[2,3-b][1,4]oxazin-7-yl}-5H,6H,7H,8H-pyrido[3,4-d]pyrimidin-2-yl)amino]-1,3-dihydro-2?6-benzothiophene-2,2-dione: To a stirred solution of tert-butyl 7-{2-[(2,2-dioxo-1,3-dihydro-2?6-benzothiophen-5-yl)amino]-5H,6H,7H,8H-pyrido[3,4-d]pyrimidin-7-yl}-8-methyl-1H,2H,3H-pyrido[2,3-b][1,4]oxazine-1-carboxylate (70.00 mg; 0.11 mmol) in DCM (5.00 mL) was added TFA (0.20 mL) in portions at room temperature. Then the resulting mixture was stirred for 1 h at 25 degrees C. under nitrogen atmosphere. The resulting mixture was concentrated under reduced pressure. The crude product was purified by Prep-HPLC with the following conditions (Column: XBridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 30% B to 60% B in 9 min., 60% B; Wave Length: 254 nm; RT1 (min.): 7; Number Of Runs: 2) to afford 5-[(7-{8-methyl-1H,2H,3H-pyrido[2,3-b][1,4]oxazin-7-yl}-5H,6H,7H,8H-pyrido[3,4-d]pyrimidin-2-yl)amino]-1,3-dihydro-2-6-benzothiophene-2,2-dione as yellow solid (13.40 mg; 26.2%).

HPLC 98.92% purity, 3.05 min. MS: m/z=465.00[M+H]⁺. ¹H NMR (300 MHz, DMSO-d6): 9.70 (s, 1H), 8.36 (s, 1H), 7.87 (d, J=2.0 Hz, 1H), 7.66 (d, J=8.0 Hz, 1H), 7.34-7.19 (m, 2H), 5.52 (s, 1H), 4.43 (d, J=25.0 Hz, 4H), 4.20 (d, J=4.6 Hz, 2H), 3.97 (s, 2H), 3.32 (s, 2H), 3.13 (s, 2H), 2.80 (s, 2H), 2.04 (s, 3H).

### Example 82: Synthesis of Compound 82 2-(3-((7-(8-methyl-2,3-dihydro-1H-pyrido[2,3-b][1,4]oxazin-7-yl)-5,6,7,8-tetrahydropyrido[3,4-d]pyrimidin-2-yl)amino)phenyl)propan-2-ol

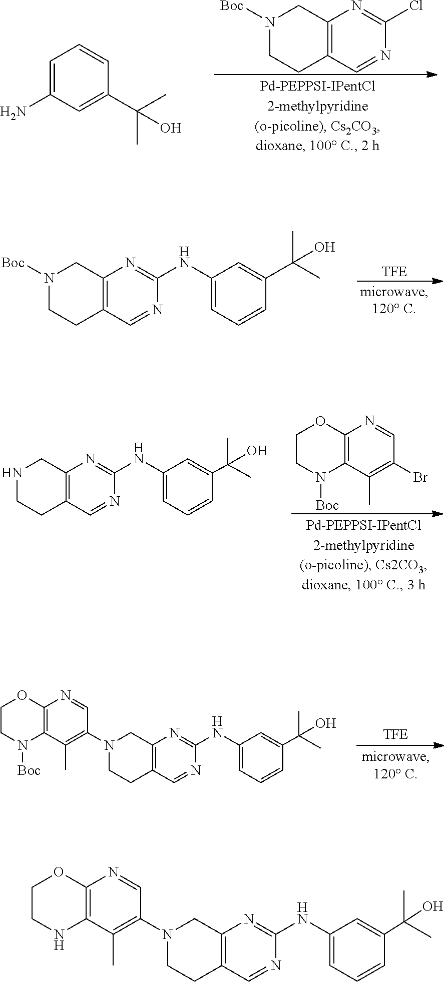

tert-butyl 2-{[3-(2-hydroxypropan-2-yl)phenyl]amino}-5H,6H,7H,8H-pyrido[3,4-d]pyrimidine-7-carboxylate: To a stirred solution of 2-(3-aminophenyl)propan-2-ol (600 mg; 3.77 mmol) and tert-butyl 2-chloro-5H,6H,7H,8H-pyrido[3,4-d]pyrimidine-7-carboxylate (1000 mg; 3.52 mmol) in 1,4-dioxane (20 mL) was added Cs₂CO₃ (2.60 g; 7.58 mmol) and Pd-PEPPSI-IPentCl 2-methylpyridine (o-picoline) (334 mg; 0.38 mmol) in portions at room temperature under N₂ atmosphere. The resulting mixture was stirred for 4 h at 100 degree C. under N₂ atmosphere. The residue was purified by silica gel column and eluted with EtOAc to afford tert-butyl 2-{[3-(2-hydroxypropan-2-yl)phenyl]amino}-5H,6H,7H,8H-pyrido[3,4-d]pyrimidine-7-carboxylate as yellow solid (1.1 g; 75.90%).

2-[3-({5H,6H,7H,8H-pyrido[3,4-d]pyrimidin-2-yl}amino)phenyl]propan-2-ol: A solution of tert-butyl 2-{[3-(2-hydroxypropan-2-yl)phenyl]amino}-5H,6H,7H,8H-pyrido[3,4-d]pyrimidine-7-carboxylate (800 mg; 2.08 mmol) in 2,2,2-trifluoroethan-1-ol (4 mL) was MW for 4 h at 120 degree C. under N₂ atmosphere. The resulting mixture was concentrated under reduced pressure to afford 2-[3-({5H,6H,7H,8H-pyrido[3,4-d]pyrimidin-2-yl}amino)phenyl]propan-2-ol as brown oil (760 mg; 82.41%).

tert-butyl 7-(2-{[3-(2-hydroxypropan-2-yl)phenyl]amino}-5H,6H,7H,8H-pyrido[3,4-d]pyrimidin-7-yl)-8-methyl-1H,2H,3H-pyrido[2,3-b][1,4]oxazine-1-carboxylate: To a stirred solution of 2-[3-({5H,6H,7H,8H-pyrido[3,4-d]pyrimidin-2-yl}amino)phenyl]propan-2-ol (750 mg; 1.69 mmol), tert-butyl 7-bromo-8-methyl-1H,2H,3H-pyrido[2,3-b][1,4]oxazine-1-carboxylate (928 mg; 2.54 mmol) in 1,4-dioxane (10 mL) was added Cs₂CO₃ (1160 mg; 3.38 mmol) and Pd-PEPPSI-IPentCl 2-methylpyridine (o-picoline) (150 mg; 0.17 mmol) in portions at room temperature under N₂ atmosphere. The resulting mixture was stirred for 4 h at 100 degree C. under N₂ atmosphere. LCMS: Complete conversion. The resulting mixture was diluted with water (15 mL), extracted with EtOAc (3×10 mL). The combined organic layers were washed with brine (15 mL) and dried over anhydrous Na₂SO₄. The resulting mixture was concentrated under reduced pressure. The residue was purified by silica gel column and eluted with 75% EtOAc in PE to afford tert-butyl 7-(2-{[3-(2-hydroxypropan-2-yl)phenyl]amino}-5H,6H,7H,8H-pyrido[3,4-d]pyrimidin-7-yl)-8-methyl-1H,2H,3H-pyrido[2,3-b][1,4]oxazine-1-carboxylate as brown solid (250 mg; 13.14%).

2-{3-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}propan-2-ol: A solution of tert-butyl 7-(2-{[3-(2-hydroxypropan-2-yl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (200 mg; 0.35 mmol) in 2,2,2-trifluoroethan-1-ol (3.00 mL) was heated under microwave for 1.5 h at 120 degree C. under N₂ atmosphere. The resulting mixture was concentrated under reduced pressure. The residue was purified by C18 flash column and eluted with 35% ACN in water (0.5% NH₃—H₂O and 0.5% NH₄HCO₃) to afford 2-{3-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}propan-2-ol as off-white solid (15.80 mg; 10.23%).

HPLC 99.35% purity, 3.23 min. MS: m/z=433.25 [M+H]⁺. ¹H NMR (400 MHz, DMSO-d6): 9.39 (s, 1H), 8.32 (s, 1H), 7.81 (t, J=2.0 Hz, 1H), 7.65-7.58 (m, 1H), 7.28 (s, 1H), 7.17 (t, J=7.9 Hz, 1H), 7.02 (d, J=7.8 Hz, 1H), 5.52 (s, 1H), 4.90 (s, 1H), 4.22-4.16 (m, 2H), 3.94 (s, 2H), 3.30 (s, 2H), 3.12 (s, 2H), 2.78 (s, 2H), 2.04 (s, 3H), 1.41 (s, 6H).

### Example 83: Synthesis of Compound 83 (2-(2-methoxyethoxy)-5-((7-(8-methyl-2,3-dihydro-1H-pyrido[2,3-b][1,4]oxazin-7-yl)-5,6,7,8-tetrahydropyrido[3,4-d]pyrimidin-2-yl)amino)phenyl)methanol

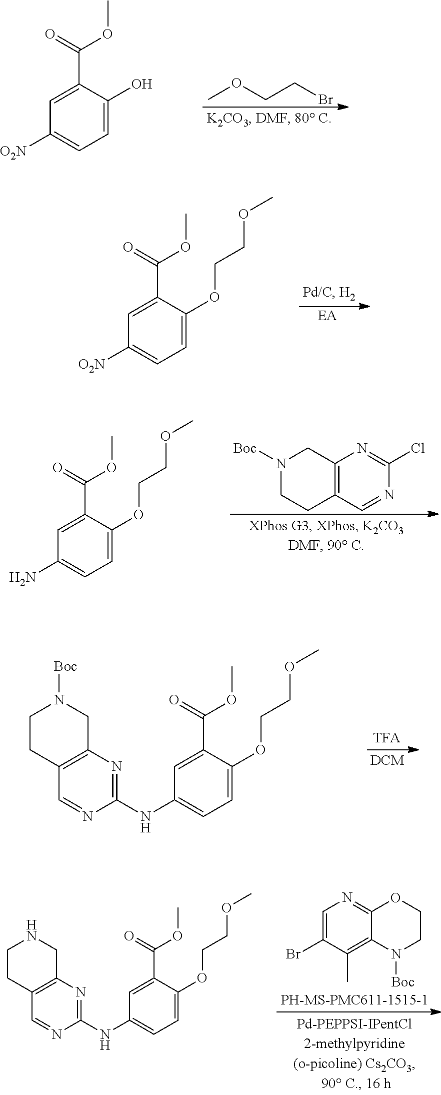

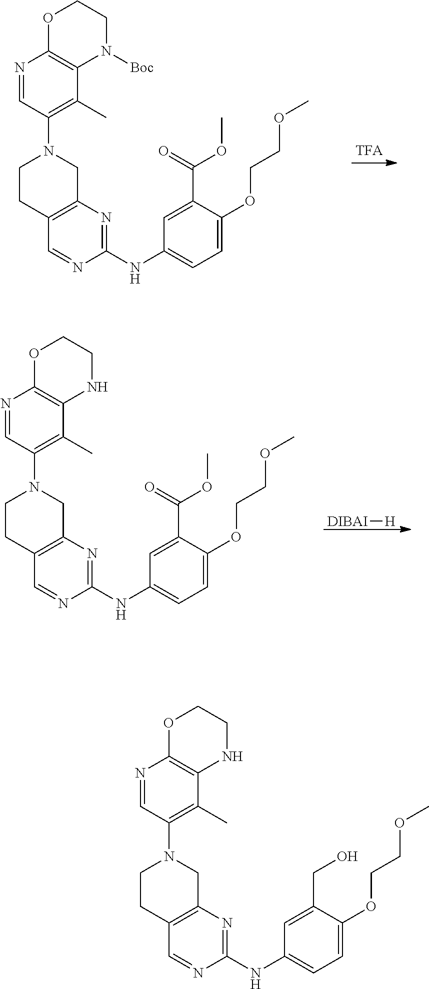

methyl 2-(2-methoxyethoxy)-5-nitrobenzoate: To a stirred solution of methyl 2-hydroxy-5-nitrobenzoate (1 g; 4.82 mmol) and 1-bromo-2-methoxyethane (850 mg; 5.81 mmol) in DMF (20 mL) was added K₂CO₃ (1.4 g; 9.62 mmol). The resulting mixture was stirred for overnight at 80 degrees C. under nitrogen atmosphere. The residue was purified by silica gel column with the following conditions (EA/PE 1:8) to afford methyl 2-(2-methoxyethoxy)-5-nitrobenzoate as white solid (1.2 g; 97.6%).

methyl 5-amino-2-(2-methoxyethoxy)benzoate: To a stirred solution of methyl 2-(2-methoxyethoxy)-5-nitrobenzoate (1.18 g; 4.62 mmol) in AcOEt (20 mL) was added Pd/C (200 mg), the resulting mixture was stirred for 2 h at room temperature under hydrogen atmosphere. The solid was filtered out. The filtrate was concentrated under vacuum to afford methyl 5-amino-2-(2-methoxyethoxy)benzoate as yellow oil (1.01 g; 95.4%).

tert-butyl 2-{[3-(methoxycarbonyl)-4-(2-methoxyethoxy)phenyl]amino}-5H,6H,7H,8H-pyrido[3,4-d]pyrimidine-7-carboxylate: To a stirred solution of methyl 5-amino-2-(2-methoxyethoxy)benzoate (1 g; 4.37 mmol) and tert-butyl 2-chloro-5H,6H,7H,8H-pyrido[3,4-d]pyrimidine-7-carboxylate (1.4 g; 5.19 mmol) in DMF (20 mL) was added X-PHOS (250 mg; 0.50 mmol), Xphos Pd G3 (350 mg; 0.41 mmol) and K₂CO₃ (1.3 g; 8.94 mmol). The resulting mixture was stirred for 2 h at 90 degrees C. under nitrogen atmosphere. The residue was purified by silica gel column with the following conditions (EA/PE 20:1) to afford tert-butyl 2-{[3-(methoxycarbonyl)-4-(2-methoxyethoxy)phenyl]amino}-5H,6H,7H,8H-pyrido[3,4-d]pyrimidine-7-carboxylate as yellow oil (1.5 g; 74.9%).

methyl 2-(2-methoxyethoxy)-5-({5H,6H,7H,8H-pyrido[3,4-d]pyrimidin-2-yl}amino)benzoate: To a stirred solution of tert-butyl 2-{[3-(methoxy carbonyl)-4-(2-methoxyethoxy)phenyl]amino}-5H,6H,7H,8H-pyrido[3,4-d]pyrimidine-7-carboxylate (1.48 g; 3.23 mmol) in DCM (30 mL) was added TFA (10 mL). The resulting mixture was stirred for 2 h at room temperature under nitrogen atmosphere. The residue was diluted with water, then adjusted to pH 6-7 with sodium bicarbonate. The resulting solution was extracted with CH₂Cl₂. The combined organic layer was dried over anhydrous sodium sulfate and concentrated under vacuum to afford methyl 2-(2-methoxyethoxy)-5-({5H,6H,7H,8H-pyrido[3,4-d]pyrimidin-2-yl}amino)benzoate as yellow solid (1.2 g; 98.7%).

tert-butyl 7-(2-{[3-(methoxycarbonyl)-4-(2-methoxyethoxy)phenyl]amino}-5H,6H,7H,8H-pyrido[3,4-d]pyrimidin-7-yl)-8-methyl-1H,2H,3H-pyrido[2,3-b][1,4]oxazine-1-carboxylate: To a stirred solution of methyl 2-(2-methoxyethoxy)-5-({5H,6H,7H,8H-pyrido[3,4-d]pyrimidin-2-yl}amino)benzoate (400 mg; 1.06 mmol) and tert-butyl 7-bromo-8-methyl-1H,2H,3H-pyrido[2,3-b][1,4]oxazine-1-carboxylate (800 mg; 1.26 mmol) in DMF (10 mL) was added Pd-PEPPSI-IPentCl 2-methylpyridine (o-picoline) (100 mg; 0.11 mmol) and Cs₂CO₃ (1 g; 2.92 mmol). The resulting mixture was stirred for overnight at 90 degrees C. under nitrogen atmosphere. The residue was purified by silica gel column with the following conditions (CH₂Cl₂/MeOH 9:1) to afford tert-butyl 7-(2-{[3-(methoxy carbonyl)-4-(2-methoxyethoxy)phenyl]amino}-5H,6H,7H,8H-pyrido[3,4-d]pyrimidin-7-yl)-8-methyl-1H,2H,3H-pyrido[2,3-b][1,4]oxazine-1-carboxylate as yellow oil (190 mg; 29.5%).

methyl 2-(2-methoxyethoxy)-5-[(7-{8-methyl-1H,2H,3H-pyrido[2,3-b][1,4]oxazin-7-yl}-5H,6H,7H,8H-pyrido[3,4-d]pyrimidin-2-yl)amino]benzoate: To a stirred solution of tert-butyl 7-(2-{[3-(methoxycarbonyl)-4-(2-methoxyethoxy)phenyl]amino}-5H,6H,7H,8H-pyrido[3,4-d]pyrimidin-7-yl)-8-methyl-1H,2H,3H-pyrido[2,3-b][1,4]oxazine-1-carboxylate (180 mg; 0.30 mmol) in DCM (4.50 mL) was added TFA (1.50 mL). The resulting mixture was stirred for 2 h at room temperature under nitrogen atmosphere. The mixture was concentrated under reduced pressure. The residue was purified by Prep-TLC (CH₂Cl₂/MeOH 6:1) to afford methyl 2-(2-methoxyethoxy)-5-[(7-{8-methyl-1H,2H,3H-pyrido[2,3-b][1,4]oxazin-7-yl}-5H,6H,7H,8H-pyrido[3,4-d]pyrimidin-2-yl)amino]benzoate as yellow solid (130 mg; 80.3%).

[2-(2-methoxyethoxy)-5-[(7-{8-methyl-1H,2H,3H-pyrido[2,3-b][1,4]oxazin-7-yl}-5H,6H,7H,8H-pyrido[3,4-d]pyrimidin-2-yl)amino]phenyl]methanol as white solid: To a stirred solution of methyl 2-(2-methoxyethoxy)-5-[(7-{8-methyl-1H,2H,3H-pyrido[2,3-b][1,4]oxazin-7-yl}-5H,6H,7H,8H-pyrido[3,4-d]pyrimidin-2-yl)amino]benzoate (120 mg; 0.22 mmol) in THF (4 mL) was added Diisobutylaluminum hydride, 1.0M in hexanes (2 mL; 2.00 mmol). The resulting mixture was stirred for 2 h at 0 degrees C. under nitrogen atmosphere. The residue was purified by Prep-HPLC with the following conditions: Column: XBridge Shield RP18 OBD Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 32% B to 62% B in 9 min., 62% B; Wave Length: 254 nm; RT1 (min.): 7; Number Of Runs: 3, to afford [2-(2-methoxyethoxy)-5-[(7-{8-methyl-1H,2H,3H-pyrido[2,3-b][1,4]oxazin-7-yl}-5H,6H,7H,8H-pyrido[3,4-d]pyrimidin-2-yl)amino]phenyl]methanol as white solid (11.50 mg).

HPLC 99.66% purity, 2.86 min. MS: m/z=479.15 [M+H]⁺. ¹H NMR (400 MHz, DMSO-d6): 9.28 (s, 1H), 8.27 (s, 1H), 7.67 (d, J=2.7 Hz, 1H), 7.58 (dd, J=8.8, 2.7 Hz, 1H), 7.28 (s, 1H), 6.86 (d, J=8.8 Hz, 1H), 5.53 (t, J=2.8 Hz, 1H), 4.92 (t, J=5.6 Hz, 1H), 4.48 (d, J=5.6 Hz, 2H), 4.19 (t, J=4.3 Hz, 2H), 4.09-4.02 (m, 2H), 3.92 (s, 2H), 3.64 (dd, J=5.6, 3.6 Hz, 2H), 3.36-3.27 (m, 5H), 3.12 (t, J=5.7 Hz, 2H), 2.77 (t, J=5.7 Hz, 2H), 2.04 (s, 3H).

### Example 84: Synthesis of Compound 84 N-(isochroman-6-yl)-7-(8-methyl-2,3-dihydro-1H-pyrido[2,3-b][1,4]oxazin-7-yl)-5,6,7,8-tetrahydropyrido[3,4-d]pyrimidin-2-amine

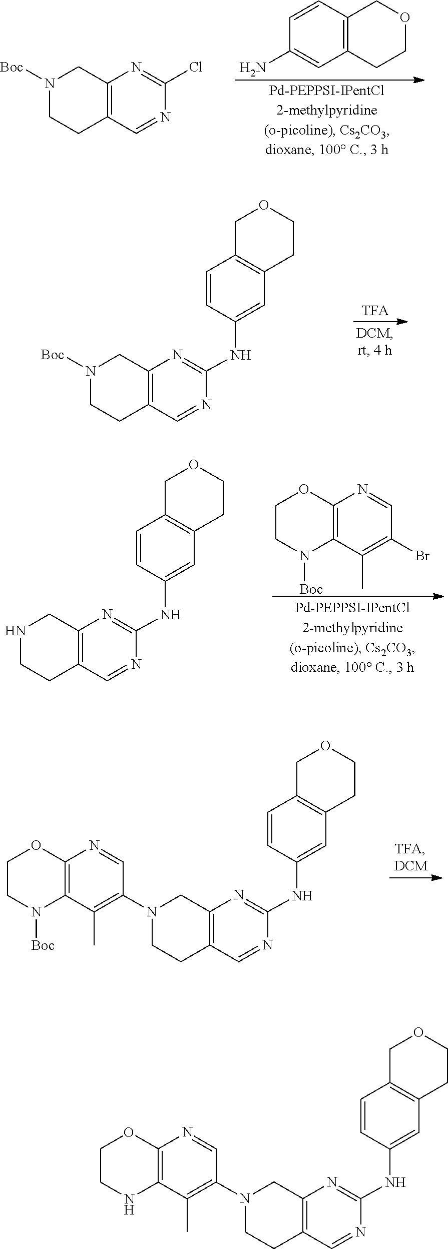

tert-butyl 2-{[2-(4-methylpiperazin-1-yl)pyridin-4-yl]amino}-5H,6H,7H,8H-pyrido[3,4-d]pyrimidine-7-carboxylate: To a solution of tert-butyl 2-chloro-5H,6H,7H,8H-pyrido[3,4-d]pyrimidine-7-carboxylate (400 mg; 1.48 mmol) and 2-(4-methylpiperazin-1-yl)pyridin-4-amine (340.00 mg; 1.48 mmol) in 1,4-dioxane (15 mL) were added Cs₂CO₃ (1017.00 mg; 2.97 mmol), and Pd-PEPPSI-IPentCl 2-methylpyridine (o-picoline) (131 mg; 0.15 mmol). The resulting mixture was stirred for 3 h at 100 degrees C. under nitrogen atmosphere. The resulting mixture was concentrated under reduced pressure. The residue was purified by silica gel column chromatography and eluted with PE:EA (1:2) to afford tert-butyl 2-{[2-(4-methylpiperazin-1-yl)pyridin-4-yl]amino}-5H,6H,7H,8H-pyrido[3,4-d]pyrimidine-7-carboxylate as yellow solid (471 mg; 56.43%).

N-(3,4-dihydro-1H-2-benzopyran-6-yl)-5H,6H,7H,8H-pyrido[3,4-d]pyrimidin-2-amine: To a stirred solution of tert-butyl 2-[(3,4-dihydro-1H-2-benzopyran-6-yl)amino]-5H,6H,7H,8H-pyrido[3,4-d]pyrimidine-7-carboxylate (400 mg; 0.79 mmol) in DCM (10 mL) was added TFA (3 mL) in portions at room temperature. The mixture was stirred for 4 hours at room temperature. The mixture was basified to pH [7] with saturated NaHCO₃ (aq.). The resulting mixture was extracted with EtOAc. The combined organic layers were dried over anhydrous Na₂SO₄. After filtration, the filtrate was concentrated under reduced pressure to afford N-(3,4-dihydro-1H-2-benzopyran-6-yl)-5H,6H,7H,8H-pyrido[3,4-d]pyrimidin-2-amine as yellow solid (200 mg; 80.3%).

tert-butyl 7-{2-[(3,4-dihydro-1H-2-benzopyran-6-yl)amino]-5H,6H,7H,8H-pyrido[3,4-d]pyrimidin-7-yl}-8-methyl-1H,2H,3H-pyrido[2,3-b][1,4]oxazine-1-carboxylate: To a solution of N-(3,4-dihydro-1H-2-benzopyran-6-yl)-5H,6H,7H,8H-pyrido[3,4-d]pyrimidin-2-amine (200 mg; 0.70 mmol) and tert-butyl 7-bromo-8-methyl-1H,2H,3H-pyrido[2,3-b][1,4]oxazine-1-carboxylate (244.93 mg; 0.70 mmol) in 1,4-dioxane (15 mL) were added Cs₂CO₃ (477.24 mg; 1.39 mmol) and Pd-PEPPSI-IPentCl 2-methylpyridine (o-picoline) (61.53 mg; 0.07 mmol). The resulting mixture was stirred for 3 h at 100 degrees C. under nitrogen atmosphere. The resulting mixture was concentrated under reduced pressure. The residue was purified by silica gel column chromatography and eluted with DCM:MEOH (10:1) to afford tert-butyl 7-{2-[(3,4-dihydro-1H-2-benzopyran-6-yl)amino]-5H,6H,7H,8H-pyrido[3,4-d]pyrimidin-7-yl}-8-methyl-1H,2H,3H-pyrido[2,3-b][1,4]oxazine-1-carboxylate as yellow solid (180 mg; 45.6%).

N-(3,4-dihydro-1H-2-benzopyran-6-yl)-7-{8-methyl-1H,2H,3H-pyrido[2,3-b][1,4]oxazin-7-yl}-5H,6H,7H,8H-pyrido[3,4-d]pyrimidin-2-amine: To a stirred solution of tert-butyl 7-{2-[(3,4-dihydro-1H-2-benzopyran-6-yl)amino]-5H,6H,7H,8H-pyrido[3,4-d]pyrimidin-7-yl}-8-methyl-1H,2H,3H-pyrido[2,3-b][1,4]oxazine-1-carboxylate (170 mg; 0.30 mmol) in DCM (10 mL) was added TFA (3 mL) in portions at room temperature. The mixture was basified to pH [7] with saturated NaHCO3 (aq.). The resulting mixture was extracted with EtOAc. The combined organic layers were dried over anhydrous Na₂SO₄. After filtration, the filtrate was concentrated under reduced pressure. The residue was purified by Prep-HPLC with the following conditions (Column: XBridge Shield RP18 OBD Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 30% B to 60% B in 9 min., 60% B; Wave Length: 254 nm; RT1 (min.): 7; Number Of Runs: 2) to afford N-(3,4-dihydro-1H-2-benzopyran-6-yl)-7-{8-methyl-1H,2H,3H-pyrido[2,3-b][1,4]oxazin-7-yl}-5H,6H,7H,8H-pyrido[3,4-d]pyrimidin-2-amine as white solid (21.90 mg).

HPLC 98.14% purity, 4.35 min. MS: m/z=431.00 [M+H]⁺. ¹H NMR (400 MHz, DMSO-d6): 9.37 (d, J=5.6 Hz, 1H), 8.31 (s, 1H), 7.51 (s, 2H), 7.27 (s, 1H), 6.90 (d, J=8.3 Hz, 1H), 5.50 (s, 1H), 4.61 (s, 2H), 4.18 (s, 3H), 3.93 (s, 2H), 3.84 (t, J=5.7 Hz, 2H), 3.10 (d, J=5.7 Hz, 2H), 2.76 (dd, J=18.4, 5.7 Hz, 5H), 2.03 (s, 3H).

### Example 85: Synthesis of Compound 85 5-((7-(8-methyl-2,3-dihydro-1H-pyrido[2,3-b][1,4]oxazin-7-yl)-5,6,7,8-tetrahydropyrido[3,4-d]pyrimidin-2-yl)amino)isoindolin-1-one

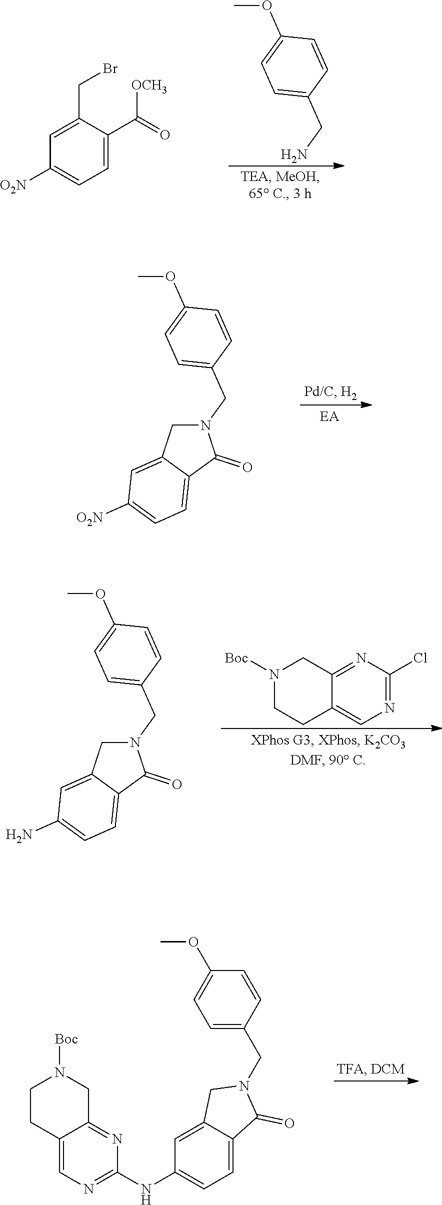

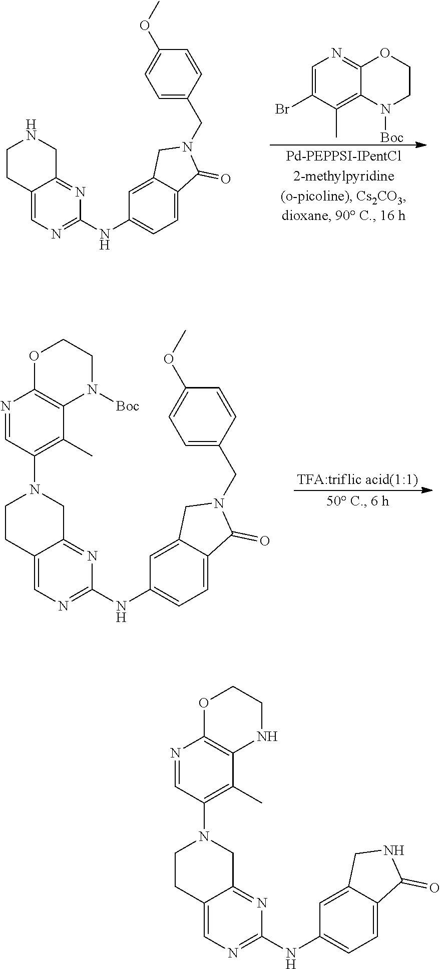 [SMILES: COc1ccc(CN2Cc3cc(Nc4ncc5c(n4)CNCC5)ccc3C2=O)cc1 ; Cc1c(Br)cnc2c1N(C(=O)OC(C)(C)C)CCO2 ; COc1ccc(CN2Cc3cc(Nc4ncc5c(n4)CN(c4cnc6c(c4C)N(C(=O)OC(C)(C)C)CCO6)CC5)ccc3C2=O)cc1 ; Cc1c(N2CCc3cnc(Nc4ccc5c(c4)CNC5=O)nc3C2)cnc2c1NCCO2]

2-[(4-methoxyphenyl)methyl]-5-nitro-2,3-dihydro-1H-isoindol-1-one: To a stirred solution of methyl 2-(bromomethyl)-4-nitrobenzoate (2 g; 6.93 mmol) and 1-(4-methoxyphenyl)methanamine (1 g; 6.93 mmol) in MeOH (20 mL) was added TEA (2.75 mL; 18.78 mmol). The resulting mixture was stirred for overnight at 65 degrees C. The resulting mixture was concentrated under vacuum. The residue was purified by silica gel column chromatography and eluted with PE/EtOAc (4:1) to afford 2-[(4-methoxyphenyl)methyl]-5-nitro-2,3-dihydro-1H-isoindol-1-one as yellow solid (1.40 g, 67.8%).

5-amino-2-[(4-methoxyphenyl)methyl]-2,3-dihydro-1H-isoindol-1-one: To a solution of 2-[(4-methoxyphenyl)methyl]-5-nitro-2,3-dihydro-1H-isoindol-1-one (1 g; 3.35 mmol) in AcOEt (20 mL) was added Pd/C (0.1 g; 0.94 mmol) in a 100 mL round-bottom flask. The mixture was hydrogenated at room temperature for 1 h under hydrogen atmosphere using a hydrogen balloon. The resulting mixture was concentrated under reduced pressure to afford 5-amino-2-[(4-methoxyphenyl)methyl]-2,3-dihydro-1H-isoindol-1-one as yellow solid (800 mg, 84.9%).

tert-butyl 2-({2-[(4-methoxyphenyl)methyl]-1-oxo-2,3-dihydro-1H-isoindol-5-yl}amino)-5H,6H,7H,8H-pyrido[3,4-d]pyrimidine-7-carboxylate: To a stirred solution of 5-amino-2-[(4-methoxyphenyl)methyl]-2,3-dihydro-1H-isoindol-1-one (500 mg; 1.78 mmol) and tert-butyl 2-chloro-5H,6H,7H,8H-pyrido[3,4-d]pyrimidine-7-carboxylate (500 mg; 1.82 mmol) in DMF (10 mL) added XPhos Pd G3 (160 mg; 0.18 mmol) and Xphos (180 mg; 0.36 mmol), K₂CO₃ (1.10 g; 7.58 mmol) under nitrogen atmosphere. The resulting mixture was stirred for 3 h at 90 degrees C. under nitrogen atmosphere. The resulting mixture was concentrated under vacuum. The residue was purified by silica gel column chromatography and eluted with PE/EtOAc (1.1) to afford tert-butyl 2-({2-[(4-methoxyphenyl)methyl]-1-oxo-2,3-dihydro-1H-isoindol-5-yl}amino)-5H,6H,7H,8H-pyrido[3,4-d]pyrimidine-7-carboxylate as yellow solid (700 mg; 46.8%).

2-[(4-methoxyphenyl)methyl]-5-({5H,6H,7H,8H-pyrido[3,4-d]pyrimidin-2-yl}amino)-2,3-dihydro-1H-isoindol-1-one: To a stirred solution of tert-butyl 2-({2-[(4-methoxyphenyl)methyl]-1-oxo-2,3-dihydro-1H-isoindol-5-yl}amino)-5H,6H,7H,8H-pyrido[3,4-d]pyrimidine-7-carboxylate (700 mg; 0.83 mmol) in DCM (10 mL) was added TFA (3 mL). The resulting mixture was stirred for 2 h at room temperature. The resulting mixture was concentrated under reduced pressure to afford 2-[(4-methoxyphenyl)methyl]-5-({5H,6H,7H,8H-pyrido[3,4-d]pyrimidin-2-yl}amino)-2,3-dihydro-TH-isoindol-1-one as brown solid (500 mg, Crude).

tert-butyl 7-[2-({2-[(4-methoxyphenyl)methyl]-1-oxo-2,3-dihydro-1H-isoindol-5-yl}amino)-5H,6H,7H,8H-pyrido[3,4-d]pyrimidin-7-yl]-8-methyl-1H,2H,3H-pyrido[2,3-b][1,4]oxazine-1-carboxylate: To a stirred solution of 2-[(4-methoxyphenyl)methyl]-5-({5H,6H,7H,8H-pyrido[3,4-d]pyrimidin-2-yl}amino)-2,3-dihydro-TH-isoindol-1-one (300 mg; 0.45 mmol) and tert-butyl 7-bromo-8-methyl-1H,2H,3H-pyrido[2,3-b][1,4]oxazine-1-carboxylate (420 mg; 1.26 mmol) in 1,4-dioxane (10 mL) was added Pd-PEPPSI-IPentCl 2-methylpyridine (o-picoline) (60 mg; 0.07 mmol) and Cs₂CO₃ (1 g; 2.92 mmol). The resulting mixture was stirred for overnight at 90 degrees C. under nitrogen atmosphere. The resulting mixture was concentrated under vacuum. The residue was purified by silica gel column chromatography and eluted with CH₂Cl₂/MeOH (15:1) to afford tert-butyl 7-[2-({2-[(4-methoxyphenyl)methyl]-1-oxo-2,3-dihydro-1H-isoindol-5-yl}amino)-5H,6H,7H,8H-pyrido[3,4-d]pyrimidin-7-yl]-8-methyl-1H,2H,3H-pyrido[2,3-b][1,4]oxazine-1-carboxylate as yellow solid (200 mg; 60.1%).

5-[(7-{8-methyl-1H,2H,3H-pyrido[2,3-b][1,4]oxazin-7-yl}-5H,6H,7H,8H-pyrido[3,4-d]pyrimidin-2-yl)amino]-2,3-dihydro-1H-isoindol-1-one: To a stirred solution of tert-butyl 7-[2-({2-[(4-methoxyphenyl)methyl]-1-oxo-2,3-dihydro-1H-isoindol-5-yl}amino)-5H,6H,7H,8H-pyrido[3,4-d]pyrimidin-7-yl]-8-methyl-1H,2H,3H-pyrido[2,3-b][1,4]oxazine-1-carboxylate (50 mg; 0.07 mmol) in TFA (1 mL) was added CF₃SO₃H (1 mL). The resulting mixture was stirred for 6 h at 50 degrees. The reaction was quenched with water/Ice at room temperature. The resulting mixture was extracted with EtOAc (3×30 mL). The combined organic layers were washed with EtOAc (3×10 mL) and dried over anhydrous Na₂SO₄. After filtration, the filtrate was concentrated under reduced pressure. The resulting mixture was concentrated under vacuum. The residue was purified by reverse flash chromatography with the following conditions. This resulted in 5-[(7-{8-methyl-1H,2H,3H-pyrido[2,3-b][1,4]oxazin-7-yl}-5H,6H,7H,8H-pyrido[3,4-d]pyrimidin-2-yl)amino]-2,3-dihydro-1H-isoindol-1-one as white solid (1.20 mg, 4.0%).

HPLC 95.75% purity, 2.99 min. M/S: m/z=430.2 [M+H]⁺. ¹H NMR (300 MHz, DMSO-d₆, ppm): 9.95 (s, 1H), 8.41 (s, 1H), 8.26 (s, 1H), 8.15-8.09 (m, 1H), 7.76 (dd, J=8.4, 1.9 Hz, 1H), 7.55 (d, J=8.3 Hz, 1H), 7.30 (s, 1H), 5.54 (s, 1H), 4.33 (s, 2H), 4.24-4.16 (m, 2H), 4.00 (s, 2H), 3.15 (s, 2H), 2.83 (s, 2H), 2.05 (s, 3H), 1.24 (s, 1H).

### Example 86: Synthesis of Compound 86 2-{[7-(4-methyl-1,2-thiazol-5-yl)-5H,6H,7H,8H-pyrido[3,4-d]pyrimidin-2-yl]amino}-6-(propan-2-yl)-4H,5H,6H,7H,8H-pyrazolo[1,5-d][1,4]diazepin-7-one; trifluoroacetic acid

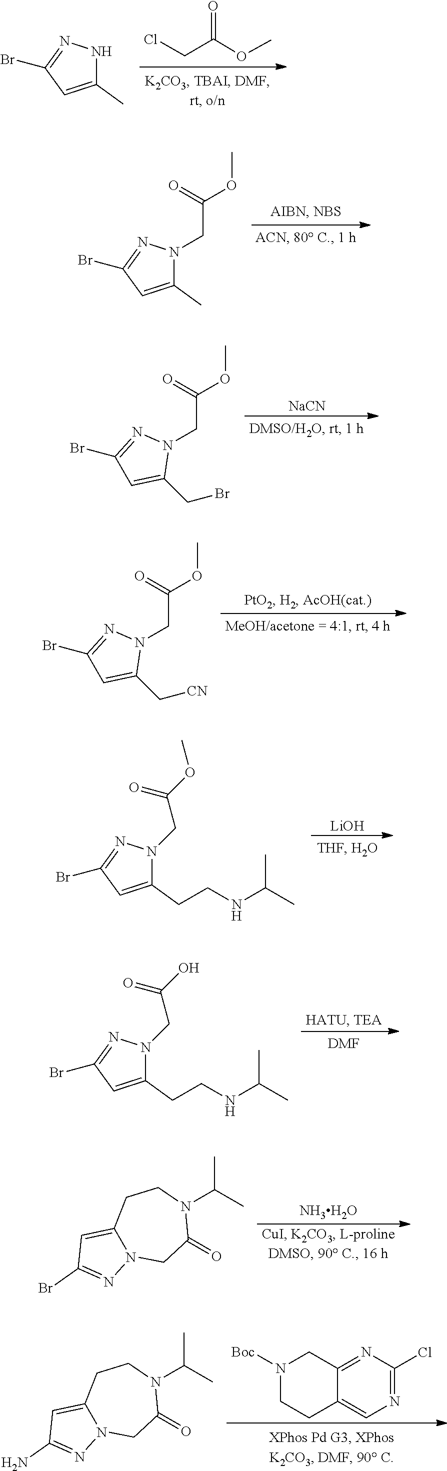

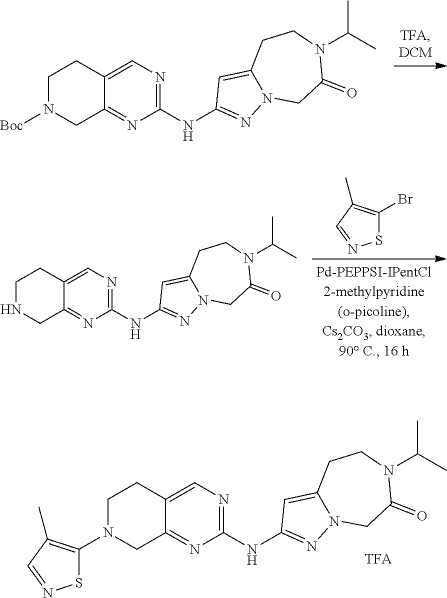

methyl 2-(3-bromo-5-methyl-1H-pyrazol-1-yl)acetate: To a stirred solution of 3-bromo-5-methyl-1H-pyrazole (50 g; 295.03 mmol) and K₂CO₃ (77.26 g; 531.07 mmol) in DMF (500 mL) was added methyl 2-chloroacetate (51 g; 446.45 mmol) dropwise at room temperature under N₂ atmosphere. The resulting mixture was stirred for overnight at 25-degree C. under N₂ atmosphere. The resulting mixture was poured into water (2 L). The mixture was extracted with EtOAc (600 mL×3). The combined organic layers were washed with brine dried over anhydrous Na₂SO₄ and concentrated under reduced pressure. The residue was purified by silica gel column and eluted with PE:EtOAc=7:1 to afford methyl 2-(3-bromo-5-methyl-1H-pyrazol-1-yl)acetate as white solid (65 g, 81.83%).

methyl 2-[3-bromo-5-(bromomethyl)-1H-pyrazol-1-yl]acetate: To a stirred solution of methyl 2-(3-bromo-5-methyl-1H-pyrazol-1-yl)acetate (40 g; 148.58 mmol) and NBS (30.62 g; 163.44 mmol) in CCl4 (400 mL) was added AIBN (25.68 g; 148.58 mmol) in portions at room temperature under N₂ atmosphere. The resulting mixture was stirred for 1 h at 80-degree C. under N₂ atmosphere. The resulting mixture was diluted with water (500 mL), extracted with EtOAc (3×250 mL). The combined organic layers were washed with brine (250 mL) and dried over anhydrous Na₂SO₄. The resulting mixture was concentrated under reduced pressure to afford methyl 2-[3-bromo-5-(bromomethyl)-1H-pyrazol-1-yl]acetate as yellow solid (90 g; Crude Product).

methyl 2-[3-bromo-5-(cyanomethyl)-1H-pyrazol-1-yl]acetate: To a stirred solution of methyl 2-[3-bromo-5-(bromomethyl)-1H-pyrazol-1-yl]acetate (90 g; 129.54 mmol) in DMSO (450 mL) and water (9 mL) was added Sodium cyanide (10.37 g; 207.26 mmol) in portions at room temperature under N₂ atmosphere. The resulting mixture was stirred for 1 h at 25-degree C. under N₂. The resulting mixture was diluted with water (1.5 L) extracted with EtOAc (3×300 mL). The combined organic layers were washed with brine (1.5 L) and dried over anhydrous Na₂SO₄. The resulting mixture was concentrated under reduced pressure. The residue was purified by silica gel column and eluted with EtOAc:PE=1:3 to afford methyl 2-[3-bromo-5-(cyanomethyl)-1H-pyrazol-1-yl]acetate as yellow solid (18 g; 40.35%).

methyl 2-(3-bromo-5-{2-[(propan-2-yl)amino]ethyl}-1H-pyrazol-1-yl)acetate: To a stirred mixture of methyl 2-[3-bromo-5-(cyanomethyl)-1H-pyrazol-1-yl]acetate (15 g; 43.56 mmol) in MeOH (50 mL) and acetone (12.5 mL) was added dioxoplatinum (4.50 g; 18.83 mmol) and AcOH (60 mg; 0.99 mmol) dropwise at room temperature under nitrogen. The resulting mixture was stirred for 1.5 h at room temperature under hydrogen (8.78 g; 4355.76 mmol) atmosphere. The resulting mixture was filtered concentrated under reduced pressure. The residue was purified by silica gel column and eluted with DCM:MeOH=20:1 to afford methyl 2-(3-bromo-5-{2-[(propan-2-yl)amino]ethyl}-1H-pyrazol-1-yl)acetate as yellow solid (8.00 g; 53.33%).

2-(3-bromo-5-{2-[(propan-2-yl)amino]ethyl}-1H-pyrazol-1-yl)acetic acid: To a stirred mixture of methyl 2-(3-bromo-5-{2-[(propan-2-yl)amino]ethyl}-1H-pyrazol-1-yl)acetate (7 g; 20.32 mmol) and methyl 2-(3-bromo-5-{2-[(propan-2-yl)amino]ethyl}-1H-pyrazol-1-yl)acetate (13 g; 37.75 mmol) in THF (100 mL) was added LiOH (5.13 g; 116.14 mmol) in water (200 mL) dropwise at room temperature. The resulting mixture was stirred for 1 h at room temperature under nitrogen atmosphere. The resulting mixture was adjust PH=5-6 with HCl (6N) and concentrated under reduced pressure to afford 2-(3-bromo-5-{2-[(propan-2-yl)amino]ethyl}-1H-pyrazol-1-yl)acetic acid as white solid (30 g; Crude) which was used for next step without further purification.

2-bromo-6-(propan-2-yl)-4H,5H,6H,7H,8H-pyrazolo[1,5-d][1,4]diazepin-7-one: To a stirred solution of 2-(3-bromo-5-{2-[(propan-2-yl)amino]ethyl}-1H-pyrazol-1-yl)acetic acid (25 g; 74.24 mmol) and HATU (29.71 g; 74.24 mmol) in MeCN (250 mL) was added TEA (16.29 mL; 111.35 mmol) in portions at room temperature under N₂ atmosphere. The resulting mixture was stirred for 1 h at 25-degree C. under N₂ atmosphere. The resulting mixture was diluted with water (1 L), extracted with EtOAc (3×300 mL). The combined organic layers were washed with brine (500 mL) and dried over anhydrous Na₂SO₄. The resulting mixture was concentrated under reduced pressure. The residue was slurred with PE:EtOAc=2:1 (30 mL) and the solid was filtered to afford 2-bromo-6-(propan-2-yl)-4H,5H,6H,7H,8H-pyrazolo[1,5-d][1,4]diazepin-7-one as white solid (11.70 g, 54.55%).

2-amino-6-(propan-2-yl)-4H,5H,6H,7H,8H-pyrazolo[1,5-d][1,4]diazepin-7-one: To a stirred solution of 2-bromo-6-(propan-2-yl)-4H,5H,6H,7H,8H-pyrazolo[1,5-d][1,4]diazepin-7-one (600 mg; 2.08 mmol) and Ammonium hydroxide solution (0.05 mL; 37.96 mmol) in DMSO (10 mL) was added CuI (100 mg; 0.50 mmol), L-proline (610 mg; 5.03 mmol) and K₂CO₃ (70 mg; 0.48 mmol). The resulting mixture was stirred for overnight at 90 degrees C. under nitrogen atmosphere. The solvent was removed under vacuum. The residue was purified by reverse flash chromatography with the following conditions: column, C18 silica gel; mobile phase, MeCN in water, 10% to 50% gradient in 10 min.; detector, UV 254 nm. This resulted in 2-amino-6-(propan-2-yl)-4H,5H,6H,7H,8H-pyrazolo[1,5-d][1,4]diazepin-7-one as yellow solid (350 mg; 80.9%).

tert-butyl 2-{[7-oxo-6-(propan-2-yl)-4H,5H,6H,7H,8H-pyrazolo[1,5-d][1,4]diazepin-2-yl]amino}-5H,6H,7H,8H-pyrido[3,4-d]pyrimidine-7-carboxylate: To a stirred solution of 2-amino-6-(propan-2-yl)-4H,5H,6H,7H,8H-pyrazolo[1,5-d][1,4]diazepin-7-one (350 mg; 1.3 mmol) and tert-butyl 2-chloro-5H,6H,7H,8H-pyrido[3,4-d]pyrimidine-7-carboxylate (270 mg; 1.3 mmol) in DMF (10 mL) was added K₂CO₃ (359 mg; 2.6 mmol) Xphos (122 mg; 0.26 mmol) and XPhos Pd G3 (110 mg; 0.13 mmol). The resulting mixture was stirred for 3 h at 90 degrees C. under nitrogen atmosphere. The resulting mixture was concentrated under vacuum. The residue was purified by silica gel column chromatography and eluted with PE/EtOAc (1:10) to afford tert-butyl 2-{[7-oxo-6-(propan-2-yl)-4H,5H,6H,7H,8H-pyrazolo[1,5-d][1,4]diazepin-2-yl]amino}-5H,6H,7H,8H-pyrido[3,4-d]pyrimidine-7-carboxylate as yellow solid (300 mg; 40.4%).

6-(propan-2-yl)-2-({5H,6H,7H,8H-pyrido[3,4-d]pyrimidin-2-yl}amino)-4H,5H,6H,7H,8H-pyrazolo[1,5-d][1,4]diazepin-7-one: To a stirred solution of tert-butyl 2-{[7-oxo-6-(propan-2-yl)-4H,5H,6H,7H,8H-pyrazolo[1,5-d][1,4]diazepin-2-yl]amino}-5H,6H,7H,8H-pyrido[3,4-d]pyrimidine-7-carboxylate (300 mg; 0.68 mmol) in DCM (10 mL) was added TFA (2 mL). The resulting mixture was stirred for 2 h at room temperature. The resulting mixture was concentrated under reduced pressure. This resulted in 6-(propan-2-yl)-2-({5H,6H,7H,8H-pyrido[3,4-d]pyrimidin-2-yl}amino)-4H,5H,6H,7H,8H-pyrazolo[1,5-d][1,4]diazepin-7-one as yellow solid (220 mg; 94.8%).

2-{[7-(4-methyl-1,2-thiazol-5-yl)-5H,6H,7H,8H-pyrido[3,4-d]pyrimidin-2-yl]amino}-6-(propan-2-yl)-4H,5H,6H,7H,8H-pyrazolo[1,5-d][1,4]diazepin-7-one; trifluoroacetic acid: To a stirred solution of 6-(propan-2-yl)-2-({5H,6H,7H,8H-pyrido[3,4-d]pyrimidin-2-yl}amino)-4H,5H,6H,7H,8H-pyrazolo[1,5-d][1,4]diazepin-7-one (100 mg; 0.29 mmol) and 5-bromo-4-methyl-1,2-thiazole (100 mg; 0.53 mmol) in 1,4-dioxane (10 mL) was added Pd2(dba)3 (30 mg; 0.03 mmol), Xantphos (40 mg; 0.07 mmol) and Cs₂CO₃ (400 mg; 1.17 mmol) under nitrogen atmosphere. The resulting mixture was stirred for overnight at 90 degrees C. under nitrogen atmosphere. The resulting mixture was concentrated under vacuum. The residue was purified by Prep-TLC, eluted with CH₂Cl₂/MeOH (10:1) to afford it. The residue was purified by reverse flash chromatography with the following conditions: Column: XBridge Shield RP18 OBD Column, 30*150 mm, 5 μm; Mobile Phase A: water (0.05% TFA), Mobile Phase B: ACN; Flow rate: 60 mL/min.; Gradient: 17% B to 44% B in 9 min., 44% B; Wave Length: 254 nm; RTT (min.): 7; Number Of Runs: 1. This resulted in 2-{[7-(4-methyl-1,2-thiazol-5-yl)-5H,6H,7H,8H-pyrido[3,4-d]pyrimidin-2-yl]amino}-6-(propan-2-yl)-4H,5H,6H,7H,8H-pyrazolo[1,5-d][1,4]diazepin-7-one; trifluoroacetic acid as brown solid (2.60 mg, 1.3%).

HPLC 82.16% purity, 6.52 min. M/S: m/z=439.2. ¹H NMR (300 MHz, DMSO-d₆, ppm): 8.51 (s, 1H), 8.31 (s, 1H), 8.14 (s, 1H), 5.04 (s, 2H), 4.55 (d, J=6.8 Hz, 2H), 3.28 (d, J=6.7 Hz, 4H), 2.77 (s, 4H), 2.04 (s, 3H), 1.10 (d, J=6.8 Hz, 6H).

### Example 87: Synthesis of Compound 87 7-(2,3-dihydro-1H-pyrido[2,3-b][1,4]oxazin-7-yl)-N-(6-methyl-5,6,7,8-tetrahydro-1,6-naphthyridin-3-yl)-5,6,7,8-tetrahydropyrido[3,4-d]pyrimidin-2-amine

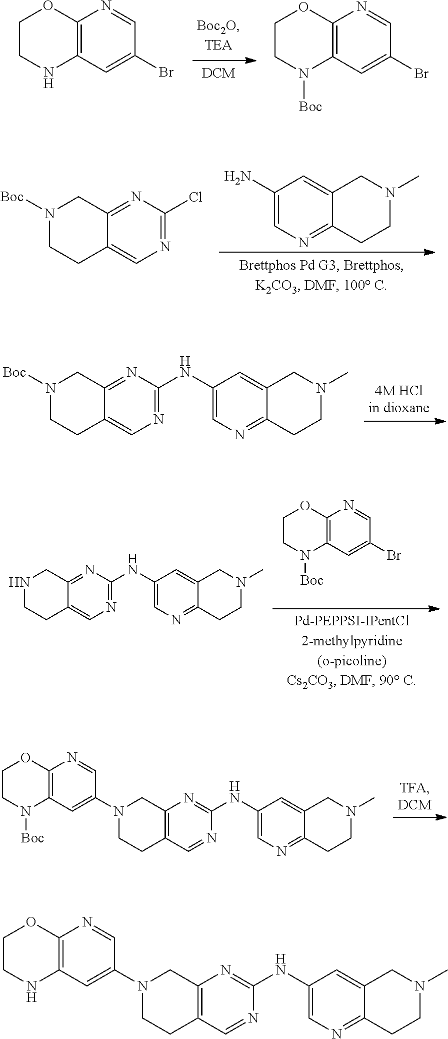

tert-butyl 7-bromo-1H,2H,3H-pyrido[2,3-b][1,4]oxazine-1-carboxylate: To a stirred solution of 7-bromo-1H,2H,3H-pyrido[2,3-b][1,4]oxazine (200 mg; 0.90 mmol) and (Boc)₂O (0.49 mL; 2.18 mmol) in DCM (10 mL) was added DMAP (80 mg; 0.62 mmol) and TEA (0.41 mL; 2.82 mmol). The resulting mixture was stirred for 2 h at room temperature under nitrogen atmosphere. The solvent was removed under vacuum, the residue was purified by silica gel column with the following conditions (EA/PE 2:3) to afford tert-butyl 7-bromo-1H,2H,3H-pyrido[2,3-b][1,4]oxazine-1-carboxylate as white solid (220 mg; 77.4%).

tert-butyl 2-[(6-methyl-5,6,7,8-tetrahydro-1,6-naphthyridin-3-yl)amino]-5H,6H,7H,8H-pyrido[3,4-d]pyrimidine-7-carboxylate: To a stirred solution of tert-butyl 2-chloro-5H,6H,7H,8H-pyrido[3,4-d]pyrimidine-7-carboxylate (1 g; 3.71 mmol) and 6-methyl-5,6,7,8-tetrahydro-1,6-naphthyridin-3-amine (900 mg; 5.24 mmol) in DMF (15 mL) was added XPhos Pd G3 (650 mg; 0.73 mmol), X-PHOS (370 mg; 0.74 mmol) and K₂CO₃ (1 g; 6.87 mmol). The resulting mixture was stirred for 2 h at 90 degrees C. under nitrogen atmosphere. The mixture was concentrated under reduced pressure. The residue was purified by Prep-HPLC (CH₂Cl₂/MeOH 4:1) to afford tert-butyl 2-[(6-methyl-5,6,7,8-tetrahydro-1,6-naphthyridin-3-yl)amino]-5H,6H,7H,8H-pyrido[3,4-d]pyrimidine-7-carboxylate as yellow solid (1.42 g; 96.6%).

6-methyl-N-{5H,6H,7H,8H-pyrido[3,4-d]pyrimidin-2-yl}-5,6,7,8-tetrahydro-1,6-naphthyridin-3-amine: To a stirred solution of tert-butyl 2-[(6-methyl-5,6,7,8-tetrahydro-1,6-naphthyridin-3-yl)amino]-5H,6H,7H,8H-pyrido[3,4-d]pyrimidine-7-carboxylate (970 mg; 2.45 mmol) in 1,4-dioxane (20 mL) was added HCl (gas) in 1,4-dioxane (10 mL; 40.00 mmol). The resulting mixture was stirred for 2 h at room temperature. This results in 6-methyl-N-{5H,6H,7H,8H-pyrido[3,4-d]pyrimidin-2-yl}-5,6,7,8-tetrahydro-1,6-naphthyridin-3-amine as yellow solid (850 mg, 95.3%).

tert-butyl 7-{2-[(6-methyl-5,6,7,8-tetrahydro-1,6-naphthyridin-3-yl)amino]-5H,6H,7H,8H-pyrido[3,4-d]pyrimidin-7-yl}-1H,2H,3H-pyrido[2,3-b][1,4]oxazine-1-carboxylate: To a stirred solution of tert-butyl 7-bromo-1H,2H,3H-pyrido[2,3-b][1,4]oxazine-1-carboxylate (200 mg; 0.63 mmol) and 6-methyl-N-{5H,6H,7H,8H-pyrido[3,4-d]pyrimidin-2-yl}-5,6,7,8-tetrahydro-1,6-naphthyridin-3-amine (240 mg; 0.66 mmol) in DME (10 mL) was added Pd-PEPPSI-IPentCl 2-methylpyridine (o-picoline) (60 mg; 0.07 mmol) and t-BuOK (400 mg; 3.39 mmol). The resulting mixture was stirred for 16 h at 80 degrees C. under nitrogen atmosphere. The residue was purified by silica gel column with the following conditions (CH₂Cl₂/MeOH 4:1) to afford tert-butyl 7-{2-[(6-methyl-5,6,7,8-tetrahydro-1,6-naphthyridin-3-yl)amino]-5H,6H,7H,8H-pyrido[3,4-d]pyrimidin-7-yl}-1H,2H,3H-pyrido[2,3-b][1,4]oxazine-1-carboxylate as yellow solid (40 mg; 11.2%).

6-methyl-N-(7-{1H,2H,3H-pyrido[2,3-b][1,4]oxazin-7-yl}-5H,6H,7H,8H-pyrido[3,4-d]pyrimidin-2-yl)-5,6,7,8-tetrahydro-1,6-naphthyridin-3-amine: To a stirred solution of tert-butyl 7-{2-[(6-methyl-5,6,7,8-tetrahydro-1,6-naphthyridin-3-yl)amino]-5H,6H,7H,8H-pyrido[3,4-d]pyrimidin-7-yl}-1H,2H,3H-pyrido[2,3-b][1,4]oxazine-1-carboxylate (35 mg; 0.06 mmol) in DCM (3 mL) was added TFA (1 mL). The resulting mixture was stirred for 2 h at room temperature under nitrogen atmosphere. The residue was purified by Prep-HPLC with the following conditions: Column: XBridge Prep OBD C18 Column, 30*150 mm, 5 μm; Mobile Phase A: water (10 mmol/L NH₄HCO₃+0.1% NH₄OH), Mobile Phase B: ACN; Flow rate: 60 mL/min.; Gradient: 10% B to 40% B in 8 min., 40% B; Wave Length: 254 nm; RT1 (min.): 7; Number Of Runs: 1, to afford 6-methyl-N-(7-{1H,2H,3H-pyrido[2,3-b][1,4]oxazin-7-yl}-5H,6H,7H,8H-pyrido[3,4-d]pyrimidin-2-yl)-5,6,7,8-tetrahydro-1,6-naphthyridin-3-amine as yellow solid (4.50 mg; 16.7%).

HPLC 99.72%, 2.06 min. M/S: m/z=431.25. ¹H NMR (400 MHz, DMSO-d6) δ 9.59 (s, 1H), 8.65 (d, J=2.5 Hz, 1H), 8.32 (s, 1H), 7.88 (d, J=2.5 Hz, 1H), 7.17 (d, J=2.7 Hz, 1H), 6.66 (d, J=2.7 Hz, 1H), 5.94 (s, 1H), 4.18 (t, J=4.4 Hz, 2H), 4.13 (s, 2H), 3.50 (s, 2H), 3.40 (t, J=5.8 Hz, 2H), 3.24 (p, J=2.3 Hz, 2H), 2.83 (t, J=5.9 Hz, 2H), 2.77 (t, J=5.8 Hz, 2H), 2.68 (t, J=6.0 Hz, 2H), 2.36 (s, 3H).

### Example 88: Synthesis of Compound 88 8-methyl-7-(2-((6-methyl-5,6,7,8-tetrahydro-1,6-naphthyridin-3-yl)amino)-5,8-dihydropyrido[3,4-d]pyrimidin-7(6H)-yl)-1H-pyrido[2,3-b][1,4]oxazin-2(3H)-one

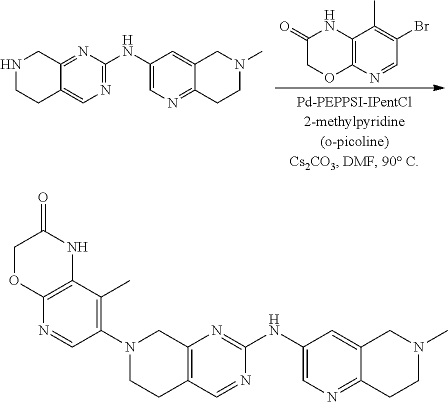

8-methyl-7-{2-[(6-methyl-5,6,7,8-tetrahydro-1,6-naphthyridin-3-yl)amino]-5H,6H,7H,8H-pyrido[3,4-d]pyrimidin-7-yl}-1H,2H,3H-pyrido[2,3-b][1,4]oxazin-2-one: To a stirred solution of 6-methyl-N-{5H,6H,7H,8H-pyrido[3,4-d]pyrimidin-2-yl}-5,6,7,8-tetrahydro-1,6-naphthyridin-3-amine (200 mg; 0.55 mmol) and 7-bromo-8-methyl-1H,2H,3H-pyrido[2,3-b][1,4]oxazin-2-one (200 mg; 0.81 mmol) in DMF (10 mL) was added Pd-PEPPSI-IPentCl 2-methylpyridine (o-picoline) (50 mg; 0.06 mmol) and Cs₂CO₃ (800 mg; 2.43 mmol). The resulting mixture was stirred for 2 h at 90 degrees C. under nitrogen atmosphere. The residue was purified by Prep-HPLC with the following conditions: Column: YMC-Actus Triart C18 ExRS, 30*150 mm, 5 μm; Mobile Phase A: water (10 mmol/L NH4HCO3+0.05% NH₄OH), Mobile Phase B: ACN; Flow rate: 60 mL/min.; Gradient: 30% B to 50% B in 8 min., 50% B; Wave Length: 254 nm; RT1 (min.): 7; Number Of Runs: 4, to afford 8-methyl-7-{2-[(6-methyl-5,6,7,8-tetrahydro-1,6-naphthyridin-3-yl)amino]-5H,6H,7H,8H-pyrido[3,4-d]pyrimidin-7-yl}-1H,2H,3H-pyrido[2,3-b][1,4]oxazin-2-one as white solid (4.30 mg; 1.7%).

HPLC 98.36% purity, 2.52 min. M/S: m/z=459.1. ¹H NMR (300 MHz, DMSO-d6) δ 10.44 (s, 1H), 9.59 (s, 1H), 8.62 (d, J=2.5 Hz, 1H), 8.36 (s, 1H), 7.89 (d, J=2.5 Hz, 1H), 7.68 (s, 1H), 4.69 (s, 2H), 4.03 (s, 2H), 3.49 (s, 2H), 3.16 (t, J=5.5 Hz, 2H), 2.82 (t, J=5.6 Hz, 4H), 2.67 (t, J=5.9 Hz, 2H), 2.35 (s, 3H), 2.23 (s, 3H).

### Example 89: Synthesis of Compound 89 6-isopropyl-2-((7-(8-methyl-2,3-dihydro-1H-pyrido[2,3-b][1,4]oxazin-7-yl)-5,6,7,8-tetrahydropyrido[3,4-d]pyrimidin-2-yl)amino)-5,6-dihydro-4H-pyrazolo[1,5-d][1,4]diazepin-7(8H)-one

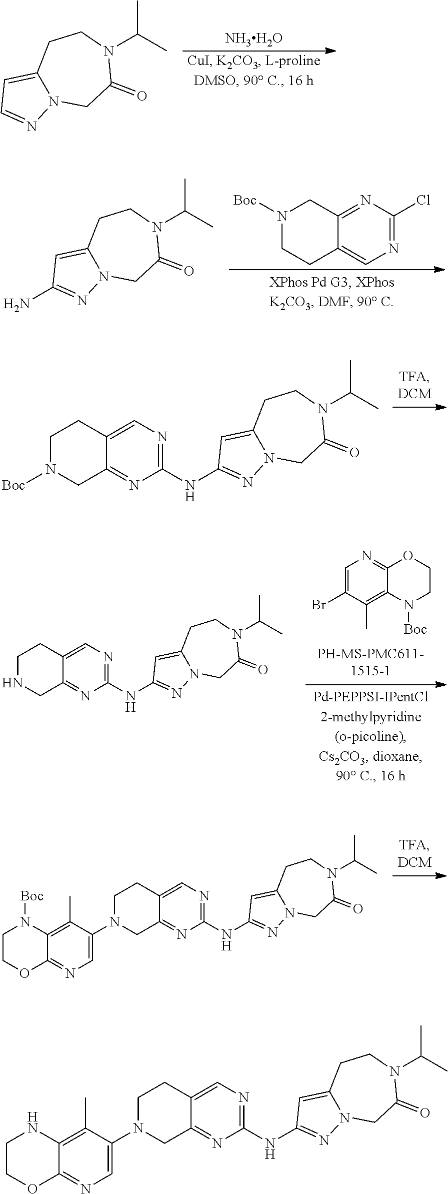

2-amino-6-(propan-2-yl)-4H,5H,6H,7H,8H-pyrazolo[1,5-d][1,4]diazepin-7-one: To a stirred solution of 2-bromo-6-(propan-2-yl)-4H,5H,6H,7H,8H-pyrazolo[1,5-d][1,4]diazepin-7-one (600 mg; 2.08 mmol) and Ammonium hydroxide solution (5 mL) in DMSO (10.00 mL) was added CuI (100 mg; 0.50 mmol) L-proline (610 mg; 5.03 mmol) and K₂CO₃ (70 mg; 0.48 mmol). The resulting mixture was stirred for overnight at 90 degrees C. under nitrogen atmosphere. The solvent was removed under vacuum. The residue was purified by reverse flash chromatography with the following conditions: column, C18 silica gel; mobile phase, MeCN in water, 10% to 50% gradient in 10 min.; detector, UV 254 nm. This resulted in 2-amino-6-(propan-2-yl)-4H,5H,6H,7H,8H-pyrazolo[1,5-d][1,4]diazepin-7-one as yellow solid (350 mg; 80.9%).

tert-butyl 2-{[7-oxo-6-(propan-2-yl)-4H,5H,6H,7H,8H-pyrazolo[1,5-d][1,4]diazepin-2-yl]amino}-5H,6H,7H,8H-pyrido[3,4-d]pyrimidine-7-carboxylate: To a stirred solution of 2-amino-6-(propan-2-yl)-4H,5H,6H,7H,8H-pyrazolo[1,5-d][1,4]diazepin-7-one (350 mg; 1.68 mmol) and tert-butyl 2-chloro-5H,6H,7H,8H-pyrido[3,4-d]pyrimidine-7-carboxylate (407 mg; 1.51 mmol) in DMF (10 mL) was added K₂CO₃ (464 mg; 3.36 mmol), Xphos (160 mg; 0.336 mmol) and XPhos Pd G3 (142 mg; 0.168 mmol). The resulting mixture was stirred for 3 h at 90 degrees C. under nitrogen atmosphere. The resulting mixture was concentrated under vacuum. The residue was purified by silica gel column chromatography and eluted with PE/EtOAc (1:10) to afford tert-butyl 2-{[7-oxo-6-(propan-2-yl)-4H,5H,6H,7H,8H-pyrazolo[1,5-d][1,4]diazepin-2-yl]amino}-5H,6H,7H,8H-pyrido[3,4-d]pyrimidine-7-carboxylate as yellow solid (300 mg; 40.4%).

6-(propan-2-yl)-2-({5H,6H,7H,8H-pyrido[3,4-d]pyrimidin-2-yl}amino)-4H,5H,6H,7H,8H-pyrazolo[1,5-d][1,4]diazepin-7-one: To a stirred solution of tert-butyl 2-{[7-oxo-6-(propan-2-yl)-4H,5H,6H,7H,8H-pyrazolo[1,5-d][1,4]diazepin-2-yl]amino}-5H,6H,7H,8H-pyrido[3,4-d]pyrimidine-7-carboxylate (300 mg; 0.68 mmol) in DCM (10 mL) was added TFA (2 mL). The resulting mixture was stirred for 2 h at room temperature. The resulting mixture was concentrated under reduced pressure. This resulted in 6-(propan-2-yl)-2-({5H,6H,7H,8H-pyrido[3,4-d]pyrimidin-2-yl}amino)-4H,5H,6H,7H,8H-pyrazolo[1,5-d][1,4]diazepin-7-one as yellow solid (220 mg; 94.8%).

tert-butyl 8-methyl-7-(2-{[7-oxo-6-(propan-2-yl)-4H,5H,6H,7H,8H-pyrazolo[1,5-d][1,4]diazepin-2-yl]amino}-5H,6H,7H,8H-pyrido[3,4-d]pyrimidin-7-yl)-1H,2H,3H-pyrido[2,3-b][1,4]oxazine-1-carboxylate: To a stirred solution of 6-(propan-2-yl)-2-({5H,6H,7H,8H-pyrido[3,4-d]pyrimidin-2-yl}amino)-4H,5H,6H,7H,8H-pyrazolo[1,5-d][1,4]diazepin-7-one (140 mg; 0.41 mmol) and tert-butyl 7-bromo-8-methyl-1H,2H,3H-pyrido[2,3-b][1,4]oxazine-1-carboxylate (200 mg; 0.60 mmol) in 1,4-dioxane (10.00 mL) was added Cs₂CO₃ (400 mg; 1.17 mmol) and Pd-PEPPSI-IPentCl 2-methylpyridine (o-picoline) (40 mg; 0.05 mmol) under nitrogen atmosphere. The resulting mixture was stirred for overnight at 80 degrees C. under nitrogen atmosphere. The resulting mixture was concentrated under vacuum. The residue was purified by Prep-TLC, eluted with CH₂Cl₂/MeOH (10:1) to afford tert-butyl 8-methyl-7-(2-{[7-oxo-6-(propan-2-yl)-4H,5H,6H,7H,8H-pyrazolo[1,5-d][1,4]diazepin-2-yl]amino}-5H,6H,7H,8H-pyrido[3,4-d]pyrimidin-7-yl)-1H,2H,3H-pyrido[2,3-b][1,4]oxazine-1-carboxylate as yellow solid (40 mg; 16.5%).

2-[(7-{8-methyl-1H,2H,3H-pyrido[2,3-b][1,4]oxazin-7-yl}-5H,6H,7H,8H-pyrido[3,4-d]pyrimidin-2-yl)amino]-6-(propan-2-yl)-4H,5H,6H,7H,8H-pyrazolo[1,5-d][1,4]diazepin-7-one: To a stirred solution of tert-butyl 8-methyl-7-(2-{[7-oxo-6-(propan-2-yl)-4H,5H,6H,7H,8H-pyrazolo[1,5-d][1,4]diazepin-2-yl]amino}-5H,6H,7H,8H-pyrido[3,4-d]pyrimidin-7-yl)-1H,2H,3H-pyrido[2,3-b][1,4]oxazine-1-carboxylate (30 mg; 0.05 mmol) in DCM (5 mL) was added BBr3 in DCM (0.20 mL; 0.20 mmol). The resulting mixture was stirred for 2 h at −60 degrees C. under nitrogen atmosphere. The resulting mixture was concentrated under vacuum. The residue was purified by reverse flash chromatography with the following conditions: Column: YMC-Actus Triart C18 ExRS, 30*150 mm, 5 μm; Mobile Phase A: water (10 mmol/L NH₄HCO₃+0.05% NH₄OH), Mobile Phase B: ACN; Flow rate: 60 mL/min.; Gradient: 20% B to 50% B in 8 min., 50% B; WaveLength: 254 nm; RT1 (min.): 7; Number Of Runs: 1. This resulted in 2-[(7-{8-methyl-1H,2H,3H-pyrido[2,3-b][1,4]oxazin-7-yl}-5H,6H,7H,8H-pyrido[3,4-d]pyrimidin-2-yl)amino]-6-(propan-2-yl)-4H,5H,6H,7H,8H-pyrazolo[1,5-d][1,4]diazepin-7-one as an off-white solid (7.30 mg; 29.0%).

HPLC 98.88%, 3.16 min. M/S: m/z=489.2. ¹H NMR (400 MHz, DMSO-d6, ppm): 9.51 (s, 1H), 8.26 (s, 1H), 7.27 (s, 1H), 6.52 (s, 1H), 5.54-5.49 (m, 1H), 4.91 (s, 2H), 4.64-4.51 (m, 1H), 4.22-4.16 (m, 2H), 3.92 (s, 2H), 3.81-3.74 (m, 2H), 3.30 (d, J=4.9 Hz, 2H), 3.14-3.06 (m, 2H), 3.02-2.95 (m, 2H), 2.80-2.73 (m, 2H), 2.03 (s, 3H), 1.12 (d, J=6.8 Hz, 6H).

### Example 90: Synthesis of Compound 90 (S)—N-(4-((1,4-dioxan-2-yl)methoxy)phenyl)-7-(8-methyl-2,3-dihydro-1H-pyrido[2,3-b][1,4]oxazin-7-yl)-5,6,7,8-tetrahydropyrido[3,4-d]pyrimidin-2-amine

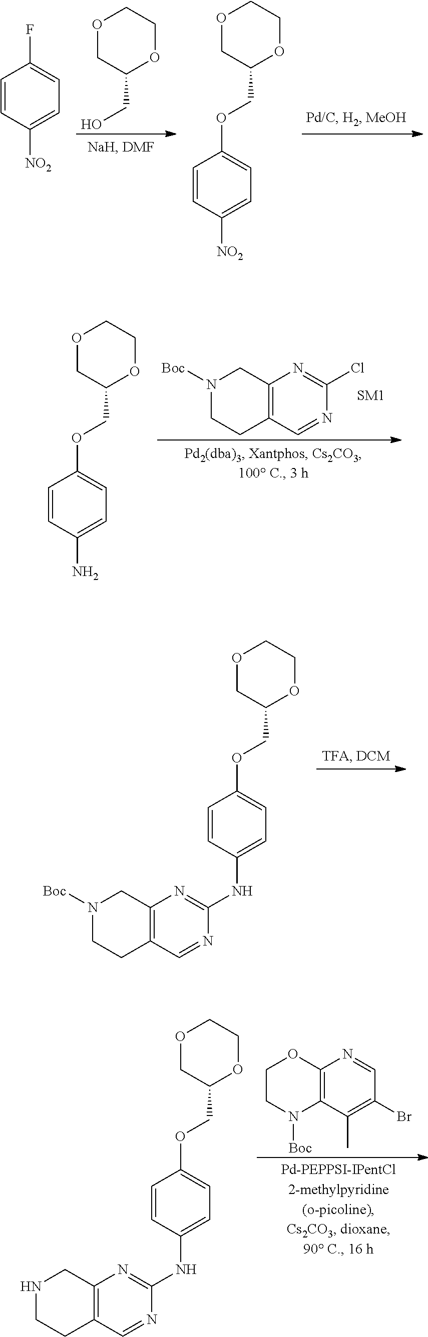

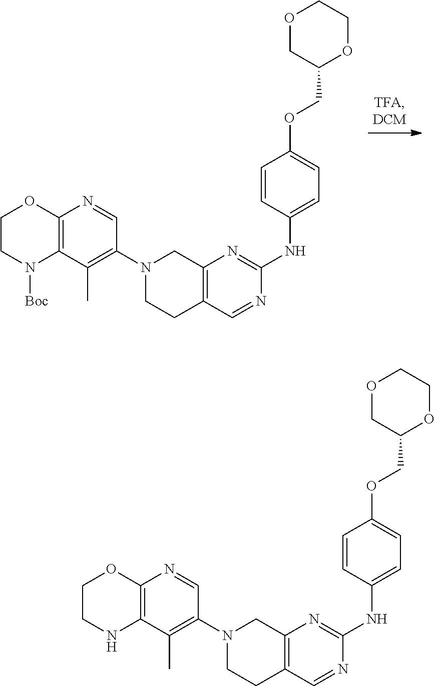 [SMILES: Cc1c(N2CCc3cnc(Nc4ccc(OC[C@@H]5COCCO5)cc4)nc3C2)cnc2c1N(C(=O)OC(C)(C)C)CCO2 ; Cc1c(N2CCc3cnc(Nc4ccc(OC[C@@H]5COCCO5)cc4)nc3C2)cnc2c1NCCO2]

(2S)-2-[(4-nitrophenoxy)methyl]-1,4-dioxane: A solution of [(2R)-1,4-dioxan-2-yl]methanol (438 mg; 3.52 mmol) and 1-fluoro-4-nitrobenzene (500 mg; 3.53 mmol) in DMF (10 mL) were added sodium hydride (282 mg; 7.05 mmol) at 0 degrees C. The resulting mixture was stirred for 3 h at 80 degrees C. The reaction was quenched with cold water, and then the mixture was extracted with EtOAc (3*15 mL). The combined organic extracts were washed with brine (10 mL) and dried over anhydrous Na₂SO₄, and concentrated under vacuum to afford (2S)-2-[(4-nitrophenoxy)methyl]-1,4-dioxane as brown oil (700 mg; 80.11%).

4-{[(2S)-1,4-dioxan-2-yl]methoxy}aniline: To a solution of (2S)-2-[(4-nitrophenoxy)methyl]-1,4-dioxane (450 mg; 1.81 mmol) in MeOH (10 mL) was added Palladium on activated carbon (188 mg; 0.18 mmol) in a round-bottom flask. The mixture was hydrogenated at room temperature for 2 h under hydrogen atmosphere using a hydrogen balloon, filtered through a Celite pad and concentrated under reduced pressure. This afforded 4-{[(2S)-1,4-dioxan-2-yl]methoxy}aniline as brown solid (350 mg, crude).

tert-butyl 2-[(4-{[(2S)-1,4-dioxan-2-yl]methoxy}phenyl)amino]-5H,6H,7H,8H-pyrido[3,4-d]pyrimidine-7-carboxylate: To a solution of tert-butyl 2-chloro-5H,6H,7H,8H-pyrido[3,4-d]pyrimidine-7-carboxylate (500 mg; 1.85 mmol) and 4-{[(2S)-1,4-dioxan-2-yl]methoxy}aniline (350 mg; 1.2 mmol) in 1,4-dioxane (15 mL) were added Cs₂CO₃ (1271.54 mg; 3.71 mmol) and Pd-PEPPSI-IPentCl 2-methylpyridine (o-picoline) (163.94 mg; 0.19 mmol). The resulting mixture was stirred for 3 h at 100 degrees C. under nitrogen atmosphere. The resulting mixture was concentrated under reduced pressure. The residue was purified by silica gel column chromatography and eluted with DCM:MEOH (9:1) to afford tert-butyl 2-[(4-{[(2S)-1,4-dioxan-2-yl]methoxy}phenyl)amino]-5H,6H,7H,8H-pyrido[3,4-d]pyrimidine-7-carboxylate as yellow solid (450 mg; 53.83%).

N-(4-{[(2S)-1,4-dioxan-2-yl]methoxy}phenyl)-5H,6H,7H,8H-pyrido[3,4-d]pyrimidin-2-amine: To a stirred solution of tert-butyl 2-[(4-{[(2S)-1,4-dioxan-2-yl]methoxy}phenyl)amino]-5H,6H,7H,8H-pyrido[3,4-d]pyrimidine-7-carboxylate (440 mg; 0.98 mmol) in DCM (5 mL) was added TFA (2 mL) in portions at room temperature. The mixture was stirred for 2 hours at room temperature. The mixture was basified to pH [7] with saturated NaHCO₃ (aq.). The resulting mixture was extracted with EtOAc. The combined organic layers were dried over anhydrous Na₂SO₄. After filtration, the filtrate was concentrated under reduced pressure. This afforded N-(4-{[(2S)-1,4-dioxan-2-yl]methoxy}phenyl)-5H,6H,7H,8H-pyrido[3,4-d]pyrimidin-2-amine (450 mg, crude).

tert-butyl 7-{2-[(4-{[(2S)-1,4-dioxan-2-yl]methoxy}phenyl)amino]-5H,6H,7H,8H-pyrido[3,4-d]pyrimidin-7-yl}-8-methyl-1H,2H,3H-pyrido[2,3-b][1,4]oxazine-1-carboxylate: To a solution of N-(4-{[(2S)-1,4-dioxan-2-yl]methoxy}phenyl)-5H,6H,7H,8H-pyrido[3,4-d]pyrimidin-2-amine (140 mg; 0.40 mmol) and tert-butyl 7-bromo-8-methyl-1H,2H,3H-pyrido[2,3-b][1,4]oxazine-1-carboxylate (141 mg; 0.40 mmol) in 1,4-dioxane (15 mL) were added Cs₂CO₃ (275 mg; 0.80 mmol) and Pd-PEPPSI-IPentCl 2-methylpyridine (o-picoline) (36 mg; 0.04 mmol). The resulting mixture was stirred for 3 h at 100 degrees C. under nitrogen atmosphere. The resulting mixture was concentrated under reduced pressure. The residue was purified by silica gel column chromatography and eluted with DCM:MEOH (9:1) to afford tert-butyl 7-{2-[(4-{[(2S)-1,4-dioxan-2-yl]methoxy}phenyl)amino]-5H,6H,7H,8H-pyrido[3,4-d]pyrimidin-7-yl}-8-methyl-1H,2H,3H-pyrido[2,3-b][1,4]oxazine-1-carboxylate as yellow solid (100 mg; 40.75%).

N-(4-{[(2S)-1,4-dioxan-2-yl]methoxy}phenyl)-7-{8-methyl-1H,2H,3H-pyrido[2,3-b][1,4]oxazin-7-yl}-5H,6H,7H,8H-pyrido[3,4-d]pyrimidin-2-amine: To a stirred solution of tert-butyl 7-{2-[(4-{[(2S)-1,4-dioxan-2-yl]methoxy}phenyl)amino]-5H,6H,7H,8H-pyrido[3,4-d]pyrimidin-7-yl}-8-methyl-1H,2H,3H-pyrido[2,3-b][1,4]oxazine-1-carboxylate (70 mg; 0.111 mmol) in DCM (5 mL) was added TFA (1 mL) in portions at room temperature. The mixture was stirred for 2 hours at room temperature. The mixture was basified to pH [7] with saturated NaHCO₃ (aq.). The resulting mixture was extracted with EtOAc. The combined organic layers were dried over anhydrous Na₂SO₄. After filtration, the filtrate was concentrated under reduced pressure. The residue was purified by Prep-HPLC with the following conditions (Column: XBridge Shield RP18 OBD Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 30% B to 60% B in 9 min., 60% B; Wave Length: 254 nm; RT1 (min.): 7; Number Of Runs: 2) to afford N-(4-{[(2S)-1,4-dioxan-2-yl]methoxy}phenyl)-7-{8-methyl-1H,2H,3H-pyrido[2,3-b][1,4]oxazin-7-yl}-5H,6H,7H,8H-pyrido[3,4-d]pyrimidin-2-amine as white solid (12.4 mg; 22.4%).

HPLC 98.82%, 3.52 min. M/S: m/z=491.2. ¹H NMR (400 MHz, DMSO-d6) δ 9.31 (s, 1H), 8.28 (s, 1H), 7.66-7.57 (m, 2H), 7.27 (s, 1H), 6.89-6.82 (m, 2H), 5.51 (d, J=2.8 Hz, 1H), 4.19 (t, J=4.4 Hz, 2H), 3.96-3.88 (m, 4H), 3.87-3.76 (m, 3H), 3.76-3.63 (m, 2H), 3.61 (dd, J=11.4, 2.5 Hz, 1H), 3.55-3.49 (m, 1H), 3.49-3.33 (m, 2H), 3.11 (t, J=5.7 Hz, 2H), 2.77 (t, J=5.6 Hz, 2H), 2.04 (s, 3H).

### Example 91: Synthesis of Compound 91 2-(2-(4-hydroxypiperidin-1-yl)-5-((7-(8-methyl-2,3-dihydro-1H-pyrido[2,3-b][1,4]oxazin-7-yl)-5,6,7,8-tetrahydropyrido[3,4-d]pyrimidin-2-yl)amino)phenyl)acetonitrile

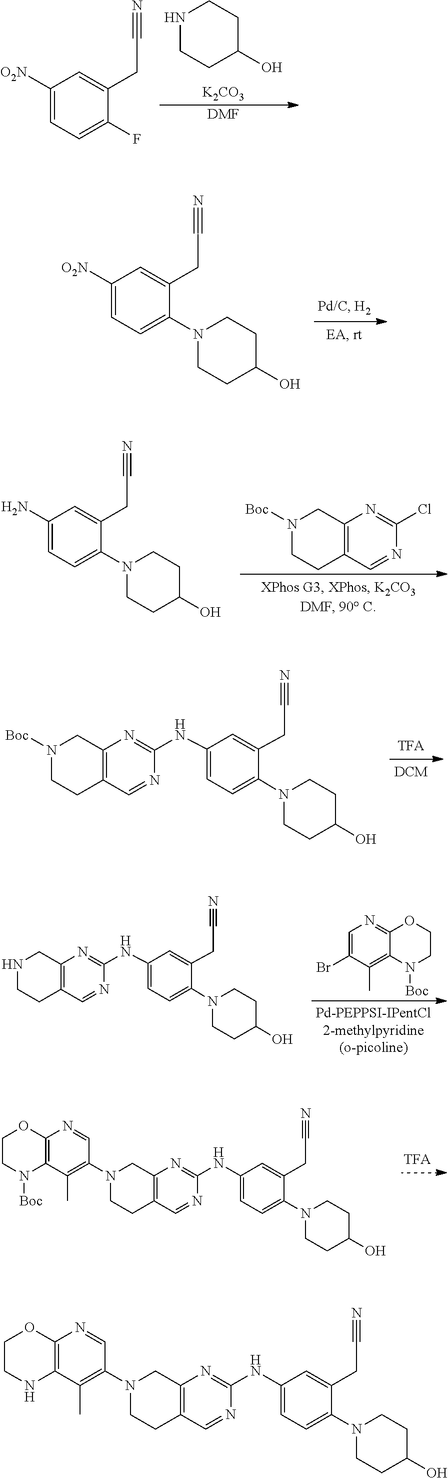

2-[2-(4-hydroxypiperidin-1-yl)-5-nitrophenyl]acetonitrile: To a stirred solution of 2-(2-fluoro-5-nitrophenyl)acetonitrile (500 mg; 2.64 mmol) and piperidin-4-ol (430.00 mg; 4.04 mmol) in DMF (10 mL) was added K₂CO₃ (750 mg; 5.16 mmol). The resulting mixture was stirred for 2 h at room temperature under nitrogen atmosphere. The solvent was removed under vacuum. The residue was purified by silica gel column with the following conditions (EA/PE 4:1) to afford 2-[2-(4-hydroxypiperidin-1-yl)-5-nitrophenyl]acetonitrile as yellow solid (400 mg; 55.8%).

2-[5-amino-2-(4-hydroxypiperidin-1-yl)phenyl]acetonitrile: To a stirred solution of 2-[2-(4-hydroxypiperidin-1-yl)-5-nitrophenyl]acetonitrile (390 mg; 1.43 mmol) in ethyl acetate (10 mL) was added Pd/C (100 mg). The resulting mixture was stirred for 2 h at room temperature under hydrogen atmosphere. The solid was filtered out. The filtrate was concentrated under vacuum to afford 2-[5-amino-2-(4-hydroxypiperidin-1-yl)phenyl]acetonitrile as yellow solid (355 mg; Crude Product).

tert-butyl 2-{[3-(cyanomethyl)-4-(4-hydroxypiperidin-1-yl)phenyl]amino}-5H,6H,7H,8H-pyrido[3,4-d]pyrimidine-7-carboxylate: To a stirred solution of 2-[5-amino-2-(4-hydroxypiperidin-1-yl)phenyl]acetonitrile (345 mg; 1.35 mmol) and tert-butyl 2-chloro-5H,6H,7H,8H-pyrido[3,4-d]pyrimidine-7-carboxylate (440 mg; 1.63 mmol) in DMF (10 mL) was added XPhos Pd G3 (240 mg; 0.27 mmol), Xphos (140.00 mg; 0.28 mmol) and K₂CO₃ (400 mg; 2.75 mmol). The resulting mixture was stirred for 2 h at 90 degrees C. under nitrogen atmosphere. The solvent was removed under vacuum. The residue was purified by silica gel column with the following conditions (EA 100%) to afford tert-butyl 2-{[3-(cyanomethyl)-4-(4-hydroxypiperidin-1-yl)phenyl]amino}-5H,6H,7H,8H-pyrido[3,4-d]pyrimidine-7-carboxylate as yellow solid (540 mg; 85.9%).

2-[2-(4-hydroxypiperidin-1-yl)-5-({5H,6H,7H,8H-pyrido[3,4-d]pyrimidin-2-yl}amino)phenyl]acetonitrile: To a stirred solution of tert-butyl 2-{[3-(cyanomethyl)-4-(4-hydroxypiperidin-1-yl)phenyl]amino}-5H,6H,7H,8H-pyrido[3,4-d]pyrimidine-7-carboxylate (530 mg; 1.14 mmol) in DCM (9 mL) was added TFA (3 mL). The resulting mixture was stirred for 2 h at room temperature under nitrogen atmosphere. The solvent was removed under vacuum. The residue was purified by silica gel column with the following conditions (CH₂Cl₂/MeOH 4:1) to afford 2-[2-(4-hydroxypiperidin-1-yl)-5-({5H,6H,7H,8H-pyrido[3,4-d]pyrimidin-2-yl}amino)phenyl]acetonitrile as yellow solid (200 mg; 48.1%).

tert-butyl 7-(2-{[3-(cyanomethyl)-4-(4-hydroxypiperidin-1-yl)phenyl]amino}-5H,6H,7H,8H-pyrido[3,4-d]pyrimidin-7-yl)-8-methyl-1H,2H,3H-pyrido[2,3-b][1,4]oxazine-1-carboxylate: To a stirred solution of 2-[2-(4-hydroxypiperidin-1-yl)-5-({5H,6H,7H,8H-pyrido[3,4-d]pyrimidin-2-yl}amino)phenyl]acetonitrile (180 mg; 0.49 mmol) and tert-butyl 7-bromo-8-methyl-1H,2H,3H-pyrido[2,3-b][1,4]oxazine-1-carboxylate (260 mg; 0.75 mmol) in DMF (10 mL) was added Pd-PEPPSI-IPentCl 2-methylpyridine (o-picoline) (80 mg; 0.09 mmol) and Cs₂CO₃ (350 mg; 1.02 mmol). The resulting mixture was stirred for overnight at 90 degrees C. under nitrogen atmosphere. The solvent was removed under vacuum. The residue was purified by silica gel column with the following conditions (CH₂Cl₂/MeOH 9:1) to afford tert-butyl 7-(2-{[3-(cyanomethyl)-4-(4-hydroxypiperidin-1-yl)phenyl]amino}-5H,6H,7H,8H-pyrido[3,4-d]pyrimidin-7-yl)-8-methyl-1H,2H,3H-pyrido[2,3-b][1,4]oxazine-1-carboxylate as yellow solid (35 mg; 11.6%).

2-[2-(4-hydroxypiperidin-1-yl)-5-[(7-{8-methyl-1H,2H,3H-pyrido[2,3-b][1,4]oxazin-7-yl}-5H,6H,7H,8H-pyrido[3,4-d]pyrimidin-2-yl)amino]phenyl]acetonitrile: To a stirred solution of tert-butyl 7-(2-{[3-(cyanomethyl)-4-(4-hydroxypiperidin-1-yl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (30 mg; 0.05 mmol) in DCM (4 mL) was added TFA (2 mL). The resulting mixture was stirred for 2 h at room temperature under nitrogen atmosphere. The solvent was removed under vacuum. The residue was purified by Prep-HPLC with the following conditions (Column: XBridge Shield RP18 OBD Column, 30*150 mm, 5 μm; Mobile Phase A: water (10 mmol/L NH4HCO3+0.05% NH₄OH), Mobile Phase B: ACN; Flow rate: 60 mL/min.; Gradient: 22% B to 57% B in 9 min., 57% B; Wave Length: 254 nm; RT1 (min.): 7; Number Of Runs: 1) to afford 2-[2-(4-hydroxypiperidin-1-yl)-5-[(7-{8-methyl-1H,2H,3H-pyrido[2,3-b][1,4]oxazin-7-yl}-5H,6H,7H,8H-pyrido[3,4-d]pyrimidin-2-yl)amino]phenyl]acetonitrile as white solid (3.4 mg; 12.6%).

HPLC 93.08%, 8.42 min. M/S: m/z=513.35. ¹H NMR (300 MHz, DMSO-d6) δ 9.52 (s, 1H), 8.32 (s, 1H), 7.92 (d, J=2.5 Hz, 1H), 7.64-7.55 (m, 1H), 7.28 (s, 1H), 7.13 (d, J=8.7 Hz, 1H), 5.54 (s, 1H), 4.66 (d, J=4.1 Hz, 1H), 4.19 (s, 2H), 3.94 (d, J=5.1 Hz, 4H), 3.59 (s, 1H), 3.13 (s, 2H), 2.87 (d, J=12.1 Hz, 2H), 2.79 (s, 2H), 2.62 (t, J=10.2 Hz, 4H), 2.04 (s, 3H), 1.84 (d, J=10.2 Hz, 2H) 1.57 (d, J=10.2 Hz, 2H).

### Example 92: Synthesis of Compound 92 N-(1,3-dihydroisobenzofuran-5-yl)-7-(8-methyl-2,3-dihydro-1H-pyrido[2,3-b][1,4]oxazin-7-yl)-5,6,7,8-tetrahydropyrido[3,4-d]pyrimidin-2-amine

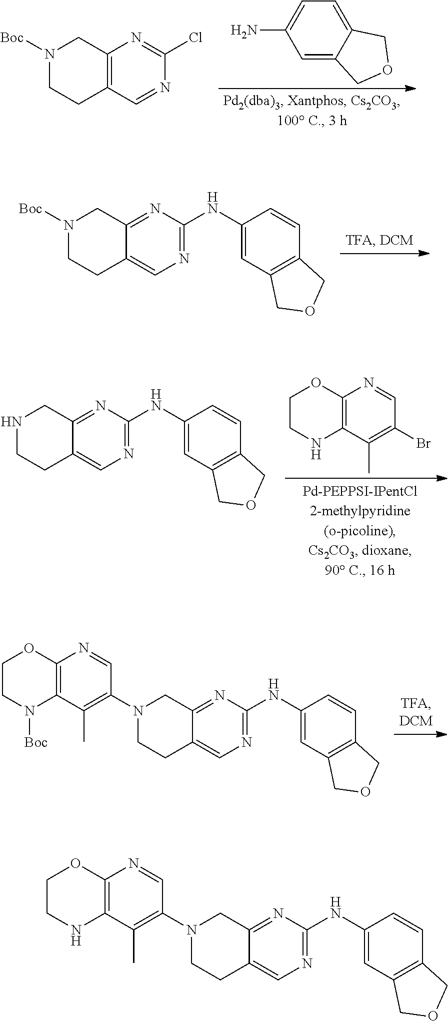

tert-butyl 2-[(1,3-dihydro-2-benzofuran-5-yl)amino]-5H,6H,7H,8H-pyrido[3,4-d]pyrimidine-7-carboxylate: To a solution of tert-butyl 2-chloro-5H,6H,7H,8H-pyrido[3,4-d]pyrimidine-7-carboxylate (500 mg; 1.854 mmol) and 1,3-dihydro-2-benzofuran-5-amine (387 mg; 2.777 mmol) in 1,4-dioxane (15 mL) were added Cs₂CO₃ (1272 mg; 3.709 mmol), tris(dibenzylideneacetone)dipalladium (189 mg; 0.186 mmol) and Xantphos (217 mg; 0.371 mmol). The resulting mixture was stirred for 3 h at 100 degrees C. under nitrogen atmosphere. The resulting mixture was concentrated under reduced pressure. The residue was purified by silica gel column chromatography and eluted with DCM/MEOH (9:1) to afford tert-butyl 2-[(1,3-dihydro-2-benzofuran-5-yl)amino]-5H,6H,7H,8H-pyrido[3,4-d]pyrimidine-7-carboxylate as yellow solid (731 mg; 102.8%).

N-(1,3-dihydro-2-benzofuran-5-yl)-5H,6H,7H,8H-pyrido[3,4-d]pyrimidin-2-amine: To a stirred solution of tert-butyl 2-[(1,3-dihydro-2-benzofuran-5-yl)amino]-5H,6H,7H,8H-pyrido[3,4-d]pyrimidine-7-carboxylate (700 mg; 1.825 mmol) in DCM (15 mL) was added TFA (3 mL) in portions at room temperature. The solvent was removed under vacuum, and the mixture was basified to pH [7] with saturated NaHCO₃ (aq.). The resulting mixture was extracted with EtOAc. The combined organic layers were washed with NaCl (aq.) and dried over anhydrous Na₂SO₄. After filtration, the filtrate was concentrated under reduced pressure to afford N-(1,3-dihydro-2-benzofuran-5-yl)-5H,6H,7H,8H-pyrido[3,4-d]pyrimidin-2-amine as yellow solid (330 mg; 48.6%).

tert-butyl 7-{2-[(1,3-dihydro-2-benzofuran-5-yl)amino]-5H,6H,7H,8H-pyrido[3,4-d]pyrimidin-7-yl}-8-methyl-1H,2H,3H-pyrido[2,3-b][1,4]oxazine-1-carboxylate: To a solution of N-(1,3-dihydro-2-benzofuran-5-yl)-5H,6H,7H,8H-pyrido[3,4-d]pyrimidin-2-amine (100 mg; 0.269 mmol) and tert-butyl 7-bromo-8-methyl-1H,2H,3H-pyrido[2,3-b][1,4]oxazine-1-carboxylate (284 mg; 0.807 mmol) in 1,4-dioxane (15 mL) were added Cs₂CO₃ (185 mg; 0.539 mmol) and Pd-PEPPSI-IPentCl 2-methylpyridine (o-picoline) (24 mg; 0.027 mmol). The resulting mixture was stirred for 3 h at 90 degrees C. under nitrogen atmosphere. The resulting mixture was concentrated under reduced pressure. The residue was purified by silica gel column chromatography and eluted with DCM:MEOH (9:1) to afford tert-butyl 7-{2-[(1,3-dihydro-2-benzofuran-5-yl)amino]-5H,6H,7H,8H-pyrido[3,4-d]pyrimidin-7-yl}-8-methyl-1H,2H,3H-pyrido[2,3-b][1,4]oxazine-1-carboxylate as yellow solid (110 mg; 78.1%).

N-(1,3-dihydro-2-benzofuran-5-yl)-7-{8-methyl-1H,2H,3H-pyrido[2,3-b][1,4]oxazin-7-yl}-5H,6H,7H,8H-pyrido[3,4-d]pyrimidin-2-amine: To a stirred solution of tert-butyl 7-{2-[(1,3-dihydro-2-benzofuran-5-yl)amino]-5H,6H,7H,8H-pyrido[3,4-d]pyrimidin-7-yl}-8-methyl-1H,2H,3H-pyrido[2,3-b][1,4]oxazine-1-carboxylate (74 mg; 0.141 mmol) and DCM (5 mL) was added tribromoborane (0.043 mL; 0.429 mmol) at −80 degrees C. under nitrogen atmosphere. Then the resulting mixture was stirred for additional 2 h at −30 degrees C. The mixture was basified to pH [7] with saturated NaHCO₃ (aq.). The resulting mixture was extracted with EtOAc. The combined organic layers were washed with NaCl (aq.) and dried over anhydrous Na₂SO₄. After filtration, the filtrate was concentrated under reduced pressure. The residue was purified by Prep-HPLC with the following conditions (Column: XSelect CSH Prep C18 OBD Column, 19*250 mm, 5 μm; Mobile Phase A: water (0.05% FA), Mobile Phase B: ACN; Flow rate: 20 m/min; Gradient: 20% B to 50% B in 8 min., 50% B; Wave Length: 254 nm; RT1 (min.): 6.8; Number Of Runs: 1) to afford N-(1,3-dihydro-2-benzofuran-5-yl)-7-{8-methyl-1H,2H,3H-pyrido[2,3-b][1,4]oxazin-7-yl}-5H,6H,7H,8H-pyrido[3,4-d]pyrimidin-2-amine as off-white solid (2.2 mg; 3.7%).

HPLC 98.64%, 3.54 min. M/S: m/z=417.05. ¹H NMR (300 MHz, DMSO-d6) δ 9.54 (s, 1H), 8.31 (s, 1H), 7.75 (s, 1H), 7.57 (d, J=8.4 Hz, 1H), 7.27 (s, 1H), 7.16 (d, J=8.4 Hz, 1H), 5.50 (s, 1H), 4.93 (d, J=8.4 Hz, 4H), 4.17 (s, 2H), 3.93 (s, 2H), 3.25-3.21 (m, 2H), 3.10 (s, 2H), 2.77 (s, 2H), 2.02 (s, 3H).

### Example 93: Synthesis of Compound 93 N-(3,3-dimethyl-1,3-dihydroisobenzofuran-5-yl)-7-(8-methyl-2,3-dihydro-1H-pyrido[2,3-b][1,4]oxazin-7-yl)-5,6,7,8-tetrahydropyrido[3,4-d]pyrimidin-2-amine

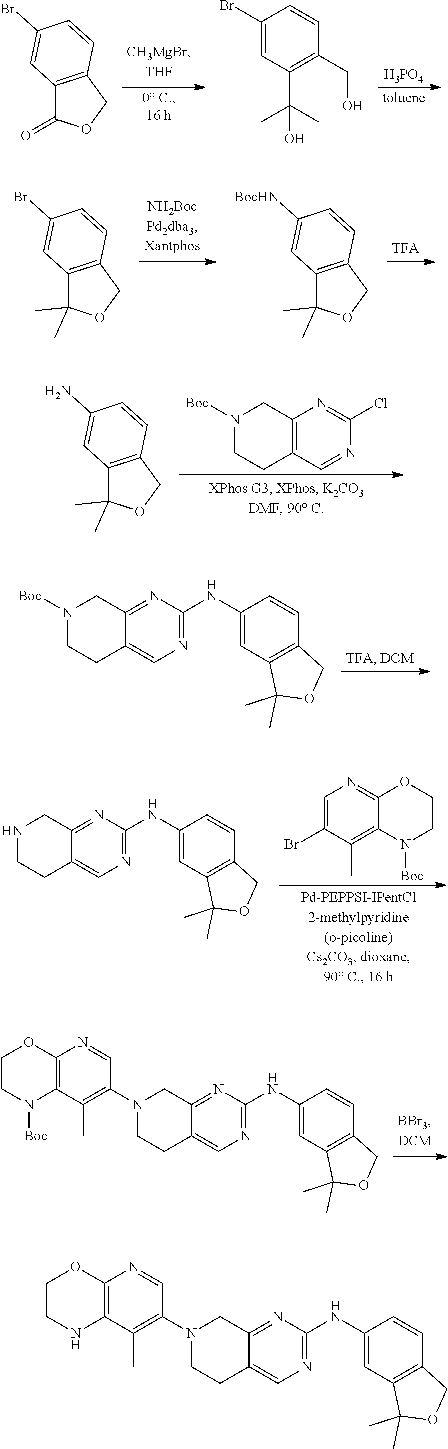

2-[5-bromo-2-(hydroxymethyl)phenyl]propan-2-ol: To a stirred solution of 6-bromo-1,3-dihydro-2-benzofuran-1-one (2.16 g; 9.63 mmol) in THF (100 mL) was added CH₃MgBr (3M in Et₂O) (10 mL; 30.00 mmol). The resulting mixture was stirred for overnight at room temperature. The resulting mixture was concentrated under vacuum. The residue was purified by silica gel column chromatography and eluted with PE/EtOAc (1:1) to afford 2-[5-bromo-2-(hydroxymethyl)phenyl]propan-2-ol as yellow oil (2.0 g; 80.5%).

6-bromo-1,1-dimethyl-1,3-dihydro-2-benzofuran: To a stirred solution of 2-[5-bromo-2-(hydroxymethyl)phenyl]propan-2-ol (1.00 g; 4.08 mmol) in toluene (15 mL) was added H₃PO₄ (3.42 mL; 62.05 mmol). The resulting mixture was stirred for 3 h at 80 degrees C. The mixture was based with NaOH (2M), then the resulting mixture was extracted with EtOAc (3×30 mL). The combined organic layers were washed with EtOAc (3×10 mL) and dried over anhydrous Na₂SO₄. After filtration, the filtrate was concentrated under reduced pressure. The residue was purified by silica gel column chromatography and eluted with PE/EtOAc (1:1) to afford 6-bromo-1,1-dimethyl-1,3-dihydro-2-benzofuran as yellow solid (800 mg; 86.3%).

tert-butyl N-(3,3-dimethyl-1,3-dihydro-2-benzofuran-5-yl)carbamate: To a stirred solution of 6-bromo-1,1-dimethyl-1,3-dihydro-2-benzofuran (400 mg; 1.76 mmol) and tert-butyl carbamate (516.00 mg; 4.18 mmol) in 1,4-dioxane (10 mL) was added Cs₂CO₃ (1.147 g; 3.52 mmol) Xantphos (260 mg; 0.44 mmol) and Pd₂(dba)₃ (200 mg; 0.21 mmol). The resulting mixture was stirred for 2 h at 100 degrees C. under nitrogen atmosphere. The resulting mixture was concentrated under vacuum. The residue was purified by silica gel column chromatography and eluted with PE/EtOAc (1:100) to afford it. This resulted in tert-butyl N-(3,3-dimethyl-1,3-dihydro-2-benzofuran-5-yl)carbamate as off-white solid (300 mg; 64.7%).

3,3-dimethyl-1,3-dihydro-2-benzofuran-5-amine: To a stirred solution of tert-butyl N-(3,3-dimethyl-1,3-dihydro-2-benzofuran-5-yl)carbamate (350.00 mg; 1.33 mmol) in DCM (10 mL) was added TFA (1.0 mL). The resulting mixture was stirred for 2 h at room temperature. The resulting mixture was concentrated under reduced pressure. This resulted in 3,3-dimethyl-1,3-dihydro-2-benzofuran-5-amine as yellow solid (210 mg; 96.8%).

tert-butyl 2-[(3,3-dimethyl-1,3-dihydro-2-benzofuran-5-yl)amino]-5H,6H,7H,8H-pyrido[3,4-d]pyrimidine-7-carboxylate: To a stirred solution of 3,3-dimethyl-1,3-dihydro-2-benzofuran-5-amine (660 mg; 4.04 mmol) and tert-butyl 2-chloro-5H,6H,7H,8H-pyrido[3,4-d]pyrimidine-7-carboxylate (1.40 g; 5.11 mmol) in DMF (10 mL) was added K₂CO₃ (2.20 g; 15.15 mmol) Xphos (400 mg; 0.80 mmol) and XPhos Pd G3 (320.00 mg; 0.36 mmol). The resulting mixture was stirred for 3 h at 90 degrees C. under nitrogen atmosphere. The resulting mixture was concentrated under vacuum. The residue was purified by silica gel column chromatography and eluted with PE/EtOAc (1:10) to afford it. This resulted in tert-butyl 2-[(3,3-dimethyl-1,3-dihydro-2-benzofuran-5-yl)amino]-5H,6H,7H,8H-pyrido[3,4-d]pyrimidine-7-carboxylate (650 mg; 40.5%) as yellow solid.

N-(3,3-dimethyl-1,3-dihydro-2-benzofuran-5-yl)-5H,6H,7H,8H-pyrido[3,4-d]pyrimidin-2-amine: To a stirred solution of tert-butyl 2-[(3,3-dimethyl-1,3-dihydro-2-benzofuran-5-yl)amino]-5H,6H,7H,8H-pyrido[3,4-d]pyrimidine-7-carboxylate (600 mg; 1.51 mmol) in DCM (10 mL) was added TFA (1 mL). The resulting mixture was stirred for 1 h at room temperature. The resulting mixture was concentrated under reduced pressure. This resulted in N-(3,3-dimethyl-1,3-dihydro-2-benzofuran-5-yl)-5H,6H,7H,8H-pyrido[3,4-d]pyrimidin-2-amine (450 mg; Crude Product) as brown solid.

tert-butyl 7-{2-[(3,3-dimethyl-1,3-dihydro-2-benzofuran-5-yl)amino]-5H,6H,7H,8H-pyrido[3,4-d]pyrimidin-7-yl}-8-methyl-1H,2H,3H-pyrido[2,3-b][1,4]oxazine-1-carboxylate: To a stirred solution of N-(3,3-dimethyl-1,3-dihydro-2-benzofuran-5-yl)-5H,6H,7H,8H-pyrido[3,4-d]pyrimidin-2-amine (400 mg; 1.29 mmol) and tert-butyl 7-bromo-8-methyl-1H,2H,3H-pyrido[2,3-b][1,4]oxazine-1-carboxylate (800 mg; 1.87 mmol) in 1,4-dioxane (10 mL) was added Cs₂CO₃ (2.20 g; 6.41 mmol) and Pd-PEPPSI-IPentCl 2-methylpyridine (o-picoline) (140 mg; 0.16 mmol) under nitrogen atmosphere. The resulting mixture was stirred for 2 h at 90 degrees C. under nitrogen atmosphere. The resulting mixture was concentrated under vacuum. The residue was purified by Prep-TLC/silica gel column chromatography and eluted with CH₂Cl₂/MeOH (10:1) to afford it. This resulted in tert-butyl 7-{2-[(3,3-dimethyl-1,3-dihydro-2-benzofuran-5-yl)amino]-5H,6H,7H,8H-pyrido[3,4-d]pyrimidin-7-yl}-8-methyl-1H,2H,3H-pyrido[2,3-b][1,4]oxazine-1-carboxylate (300 mg; 37.6%) as yellow solid.

N-(3,3-dimethyl-1,3-dihydro-2-benzofuran-5-yl)-7-{8-methyl-1H,2H,3H-pyrido[2,3-b][1,4]oxazin-7-yl}-5H,6H,7H,8H-pyrido[3,4-d]pyrimidin-2-amine: To a stirred solution of tert-butyl 7-{2-[(3,3-dimethyl-1,3-dihydro-2-benzofuran-5-yl)amino]-5H,6H,7H,8H-pyrido[3,4-d]pyrimidin-7-yl}-8-methyl-1H,2H,3H-pyrido[2,3-b][1,4]oxazine-1-carboxylate (180 mg; 0.29 mmol) in DCM (10 mL) was added TFA (1.0 mL). The resulting mixture was stirred for 2 h at room temperature. The resulting mixture was concentrated under vacuum. The residue was purified by silica gel column chromatography and eluted with CH₂Cl₂/MeOH (15:1) to afford crude product. The crude product was purified by reverse flash chromatography with the following conditions: Column: Xselect CSH OBD Column 30*150 mm, 5 μm, n; Mobile Phase A: water (10 mmol/L NH₄HCO₃+0.05% NH₄OH), Mobile Phase B: ACN; Flow rate: 60 mL/min.; Gradient: 30% B to 50% B in 10 min., 50% B; Wave Length: 254 nm; RTT (min.): 8.55; Number Of Runs: 4. This resulted in N-(3,3-dimethyl-1,3-dihydro-2-benzofuran-5-yl)-7-{8-methyl-1H,2H,3H-pyrido[2,3-b][1,4]oxazin-7-yl}-5H,6H,7H,8H-pyrido[3,4-d]pyrimidin-2-amine (37 mg; 28.1%) as off-white solid.

HPLC 98.44% purity, 3.87 min. M/S: m/z=445.25. ¹H NMR (300 MHz, DMSO-d6, ppm): 9.52 (s, 1H), 8.34 (s, 1H), 7.65 (d, J=1.9 Hz, 1H), 7.59 (dd, J=8.2, 1.9 Hz, 1H), 7.30 (s, 1H), 7.13 (d, J=8.2 Hz, 1H), 5.58-5.51 (m, 1H), 4.90 (s, 2H), 4.25-4.16 (m, 2H), 3.96 (s, 2H), 3.32 (s, 2H), 3.18-3.08 (m, 2H), 2.81 (d, J=5.8 Hz, 2H), 2.05 (s, 3H), 1.41 (s, 6H).

### Example 94: Synthesis of Compound 94 7-(8-methyl-2,3-dihydro-1H-pyrido[2,3-b][1,4]oxazin-7-yl)-N-(4-((tetrahydro-2H-pyran-4-yl)oxy)phenyl)-5,6,7,8-tetrahydropyrido[3,4-d]pyrimidin-2-amine

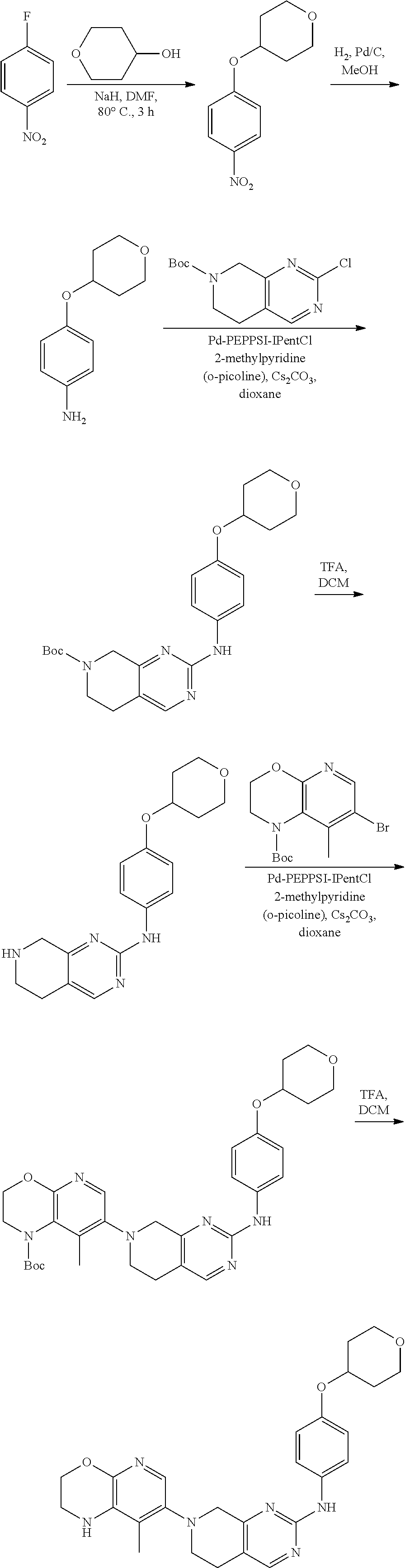

4-(4-nitrophenoxy)oxane: To a stirred solution of oxan-4-ol (869 mg; 8.083 mmol; 1.2 eq.) and DMF (15 mL) was added sodium hydride (539 mg; 13.475 mmol) at 0 degrees C. under nitrogen atmosphere. The resulting mixture was stirred for 10 min. at 0 degrees C. under nitrogen atmosphere. After 10 min., 1-fluoro-4-nitrobenzene (1 g; 6.733 mmol) was added to the above solution at 0 degrees C. under nitrogen atmosphere. Then the resulting mixture was stirred for additional 3 h at 80 degrees C. The reaction was quenched by the addition of H₂O at room temperature and extracted by EA, the combined organic layers were washed with brine and dried over anhydrous Na₂SO₄. After filtration, the filtrate was concentrated under reduced pressure. The residue was purified by silica gel column chromatography and eluted with DCM:MeOH (10:1) to afford 4-(4-nitrophenoxy)oxane as yellow solid (967 mg; 61.1%).

4-(oxan-4-yloxy)aniline: To a solution of 4-(4-nitrophenoxy)oxane (967 mg; 4.256 mmol) in MeOH (10 mL) was added Palladium on activated carbon (100 mg) in a round bottom flask. The mixture was hydrogenated at room temperature for 2 h under hydrogen atmosphere using a hydrogen balloon, filtered through a Celite pad and concentrated under reduced pressure. The resulting mixture was filtered and the filter cake was washed with 300 mL of EA. The filtrate was concentrated under reduced pressure to afford 4-(oxan-4-yloxy)aniline as brown solid (884 mg, crude).

tert-butyl 2-{[4-(oxan-4-yloxy)phenyl]amino}-5H,6H,7H,8H-pyrido[3,4-d]pyrimidine-7-carboxylate: To a solution of tert-butyl 2-chloro-5H,6H,7H,8H-pyrido[3,4-d]pyrimidine-7-carboxylate (500 mg; 1.854 mmol) and 4-(oxan-4-yloxy)aniline (434 mg; 1.855 mmol) in 1,4-dioxane (15 mL) were added Cs₂CO₃ (1272 mg; 3.709 mmol) and Pd-PEPPSI-IPentCl 2-methylpyridine (o-picoline) (164 mg; 0.185 mmol). The resulting mixture was stirred for 3 h at 100 degrees C. under nitrogen atmosphere. The resulting mixture was concentrated under reduced pressure. The residue was purified by silica gel column chromatography and eluted with DCM:MEOH (9:1) to afford tert-butyl 2-{[4-(oxan-4-yloxy)phenyl]amino}-5H,6H,7H,8H-pyrido[3,4-d]pyrimidine-7-carboxylate as yellow solid (420 mg; 47.2%).

N-[4-(oxan-4-yloxy)phenyl]-5H,6H,7H,8H-pyrido[3,4-d]pyrimidin-2-amine: To a stirred solution of tert-butyl 2-{[4-(oxan-4-yloxy)phenyl]amino}-5H,6H,7H,8H-pyrido[3,4-d]pyrimidine-7-carboxylate (370 mg; 0.770 mmol) in DCM (10 mL) was added TFA (1 mL) in portions at room temperature. The mixture was basified to pH [7] with saturated NaHCO₃ (aq.). The resulting mixture was extracted with EtOAc. The combined organic layers were dried over anhydrous Na₂SO₄. After filtration, the filtrate was concentrated under reduced pressure to afford N-[4-(oxan-4-yloxy)phenyl]-5H,6H,7H,8H-pyrido[3,4-d]pyrimidin-2-amine as yellow solid (397 mg, crude).

tert-butyl 8-methyl-7-(2-{[4-(oxan-4-yloxy)phenyl]amino}-5H,6H,7H,8H-pyrido[3,4-d]pyrimidin-7-yl)-1H,2H,3H-pyrido[2,3-b][1,4]oxazine-1-carboxylate: To a solution of N-[4-(oxan-4-yloxy)phenyl]-5H,6H,7H,8H-pyrido[3,4-d]pyrimidin-2-amine (200 mg; 0.59 mmol) and tert-butyl 7-bromo-8-methyl-1H,2H,3H-pyrido[2,3-b][1,4]oxazine-1-carboxylate (208 mg; 0.59 mmol) in 1,4-dioxane (15 mL) were added Cs₂CO₃ (405 mg; 1.18 mmol) and Pd-PEPPSI-IPentCl 2-methylpyridine (o-picoline) (52 mg; 0.06 mmol). The resulting mixture was stirred for 3 h at 90 degrees C. under nitrogen atmosphere. The resulting mixture was concentrated under reduced pressure. The residue was purified by silica gel column chromatography and eluted with DCM:MEOH (9:1) to afford tert-butyl 8-methyl-7-(2-{[4-(oxan-4-yloxy)phenyl]amino}-5H,6H,7H,8H-pyrido[3,4-d]pyrimidin-7-yl)-1H,2H,3H-pyrido[2,3-b][1,4]oxazine-1-carboxylate as yellow solid (87 mg, 21.96%).

7-{8-methyl-1H,2H,3H-pyrido[2,3-b][1,4]oxazin-7-yl}-N-[4-(oxan-4-yloxy)phenyl]-5H,6H,7H,8H-pyrido[3,4-d]pyrimidin-2-amine: To a stirred solution of tert-butyl 8-methyl-7-(2-{[4-(oxan-4-yloxy)phenyl]amino}-5H,6H,7H,8H-pyrido[3,4-d]pyrimidin-7-yl)-1H,2H,3H-pyrido[2,3-b][1,4]oxazine-1-carboxylate (80 mg; 0.120 mmol) and DCM (10 mL) was added tribromoborane (0.036 mL; 0.360 mmol) dropwise at −78 degrees C. under nitrogen atmosphere. The resulting mixture was stirred for 1 h at −30 degrees C. under nitrogen atmosphere 1 h. The mixture to pH [7] with conc saturated NaHCO₃ (aq.) and extracted by EA. The combined organic layers were washed with brine and dried over anhydrous Na₂SO₄. After filtration, the filtrate was concentrated under reduced pressure. The residue was purified by Prep-HPLC with the following conditions (Column: XBridge Shield RP18 OBD Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 26% B to 56% B in 9 min., 56% B; Wave Length: 254 nm; RTT (min.): 7; Number Of Runs: 1) to afford 7-{8-methyl-1H,2H,3H-pyrido[2,3-b][1,4]oxazin-7-yl}-N-[4-(oxan-4-yloxy)phenyl]-5H,6H,7H,8H-pyrido[3,4-d]pyrimidin-2-amine as yellow solid (7 mg; 12.2%).

HPLC 98.92%, 3.81 min. M/S: m/z=475.35. ¹H NMR (400 MHz, DMSO-d6) 9.30 (s, 1H), 8.28 (s, 1H), 7.62 (d, J=9.1 Hz, 2H), 7.27 (s, 1H), 6.89 (d, J=9.1 Hz, 2H), 5.51 (s, 1H), 4.44 (d, J=4.3 Hz, 1H), 4.19 (s, 2H), 3.92 (s, 2H), 3.89-3.79 (m, 3H), 3.53-3.43 (m, 2H), 3.30-3.27 (m, 2H), 3.10 (d, J=5.6 Hz, 2H), 2.77 (s, 2H), 2.03 (s, 3H), 1.94 (d, J=12.7 Hz, 2H), 1.60-1.52 (m, 1H).

### Example 95: Synthesis of Compound 95 7-(8-methyl-2,3-dihydro-[1,4]dioxino[2,3-b]pyridin-7-yl)-N-(6-methyl-5,6,7,8-tetrahydro-1,6-naphthyridin-3-yl)-5,6,7,8-tetrahydropyrido[3,4-d]pyrimidin-2-amine

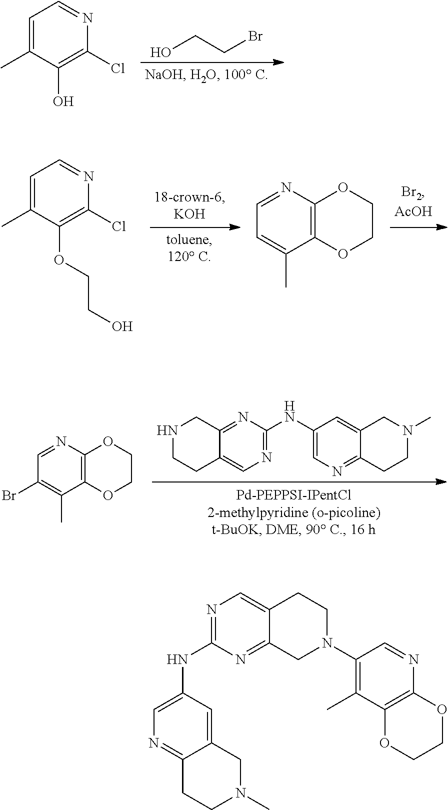

2-[(2-chloro-4-methylpyridin-3-yl)oxy]ethan-1-ol: To a stirred solution of 2-chloro-4-methylpyridin-3-ol (1.00 g; 6.62 mmol) in NaOH in water (1 M) (20.00 mL) was added 2-bromoethan-1-ol (1.30 g; 9.88 mmol) at room temperature. The resulting mixture was stirred for 4 h at 100 degrees C. The resulting solution was extracted with 3×30 mL of EA and the organic layers combined, washing with 3×30 mL brine, drying over Na₂SO₄, and concentrated under vacuum. The resulting mixture was concentrated under vacuum. This resulted in 2-[(2-chloro-4-methylpyridin-3-yl)oxy]ethan-1-ol as colorless oil (430 mg; 34.6%).

8-methyl-2H,3H-[1,4]dioxino[2,3-b]pyridine: To a stirred solution of 2-[(2-chloro-4-methylpyridin-3-yl)oxy]ethan-1-ol (350 mg; 1.87 mmol) in Toluene (10 mL) was added KOH (200 mg; 3.39 mmol) and 18-crown-6 (300 mg; 1.08 mmol) at room temperature. The resulting mixture was stirred for 1 h at 130 degrees C. The resulting mixture was concentrated under vacuum. The residue was purified by silica gel column chromatography eluted with PE/A (1:1) to afford 8-methyl-2H,3H-[1,4]dioxino[2,3-b]pyridine as white solid (220 mg; 78.0%).

7-bromo-8-methyl-2H,3H-[1,4]dioxino[2,3-b]pyridine: To a stirred solution of 8-methyl-2H,3H-[1,4]dioxino[2,3-b]pyridine (220 mg; 2.91 mmol) in AcOH (10.00 mL) was added Br₂ (0.5 mL) at room temperature. The resulting mixture was stirred for 1 h at room temperature. The resulting mixture was concentrated under vacuum. The residue was purified by silica gel column chromatography eluted with PE/EA (3:1) to afford. This resulted in 7-bromo-8-methyl-2H,3H-[1,4]dioxino[2,3-b]pyridine as white solid (200 mg; 62.2%).

6-methyl-N-(7-{8-methyl-2H,3H-[1,4]dioxino[2,3-b]pyridin-7-yl}-5H,6H,7H,8H-pyrido[3,4-d]pyrimidin-2-yl)-5,6,7,8-tetrahydro-1,6-naphthyridin-3-amine: To a stirred solution of 7-bromo-8-methyl-2H,3H-[1,4]dioxino[2,3-b]pyridine (210 mg; 0.87 mmol) and 6-methyl-N-{5H,6H,7H,8H-pyrido[3,4-d]pyrimidin-2-yl}-5,6,7,8-tetrahydro-1,6-naphthyridin-3-amine (150 mg; 0.51 mmol) in DME (20 mL) was added Pd-PEPPSI-IPentCl 2-methylpyridine (o-picoline) (60 mg; 0.07 mmol) and t-BuOK (50.00 mg; 0.42 mmol) at room temperature under nitrogen atmosphere. The resulting mixture was stirred for overnight at 80 degrees C. under nitrogen atmosphere. The resulting mixture was concentrated under vacuum. The residue was purified by silica gel column chromatography eluted with DCM/MeOH (5:1) to afford 6-methyl-N-(7-{8-methyl-2H,3H-[1,4]dioxino[2,3-b]pyridin-7-yl}-5H,6H,7H,8H-pyrido[3,4-d]pyrimidin-2-yl)-5,6,7,8-tetrahydro-1,6-naphthyridin-3-amine as white solid (5.9 mg; 1.5%).

HPLC 97.37%, 2.88 min. M/S: m/z=446.2. ¹H NMR (300 MHz, DMSO-d6) ppm 9.63 (s, 1H), 8.65 (s, 1H), 8.37 (s, 1H), 7.93 (s, 1H), 7.62 (s, 1H), 4.35 (d, J=4.9 Hz, 2H), 4.28 (d, J=4.9 Hz, 2H), 4.03 (s, 2H), 3.62 (s, 2H), 3.18 (t, J=5.6 Hz, 2H), 2.87-2.79 (m, 6H), 2.44 (s, 3H), 2.15 (s, 2H), 1.25 (s, 1H).

### Example 96: Synthesis of Compound 96 2-methyl-5-((7-(8-methyl-2,3-dihydro-1H-pyrido[2,3-b][1,4]oxazin-7-yl)-5,6,7,8-tetrahydropyrido[3,4-d]pyrimidin-2-yl)amino)isoindolin-1-one

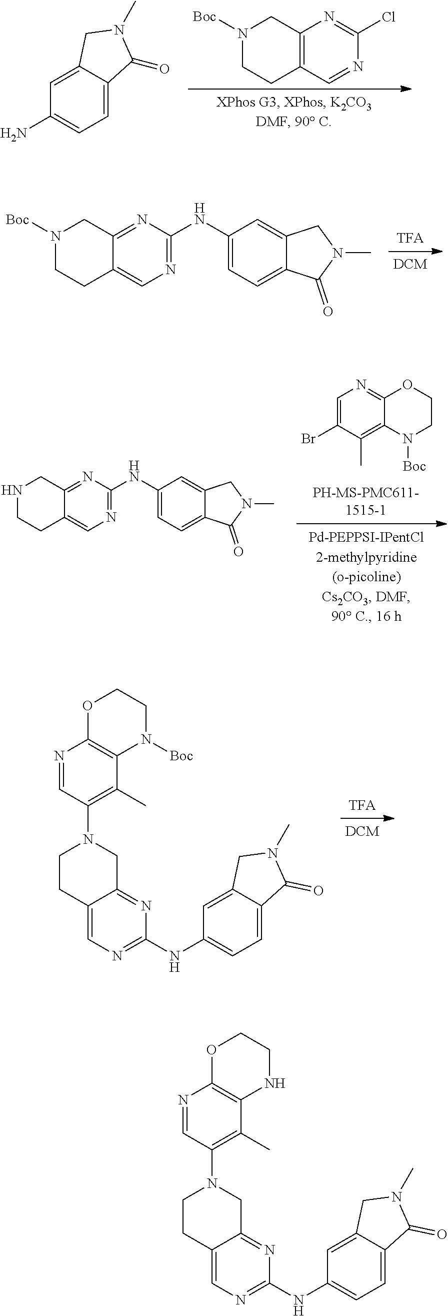

tert-butyl 2-[(2-methyl-1-oxo-2,3-dihydro-1H-isoindol-5-yl)amino]-5H,6H,7H,8H-pyrido[3,4-d]pyrimidine-7-carboxylate: To a stirred solution of 5-amino-2-methyl-2,3-dihydro-1H-isoindol-1-one (300 mg; 1.76 mmol) and tert-butyl 2-chloro-5H,6H,7H,8H-pyrido[3,4-d]pyrimidine-7-carboxylate (570 mg; 2.11 mmol) in DMF (10 mL) was added XPhos Pd G3 (160 mg; 0.18 mmol), X-PHOS (90 mg; 0.18 mmol) and K₂CO₃ (520 mg; 3.57 mmol). The resulting mixture was stirred for 2 h at 90 degrees C. under nitrogen atmosphere. The mixture was concentrated under reduced pressure. The residue was purified by Prep-TLC (CH₂Cl₂/MeOH 9:1) to afford tert-butyl 2-[(2-methyl-1-oxo-2,3-dihydro-1H-isoindol-5-yl)amino]-5H,6H,7H,8H-pyrido[3,4-d]pyrimidine-7-carboxylate as white solid (620 mg; 89.2%).

2-methyl-5-({5H,6H,7H,8H-pyrido[3,4-d]pyrimidin-2-yl}amino)-2,3-dihydro-1H-isoindol-1-one: To a stirred solution of tert-butyl 2-[(2-methyl-1-oxo-2,3-dihydro-1H-isoindol-5-yl)amino]-5H,6H,7H,8H-pyrido[3,4-d]pyrimidine-7-carboxylate (400 mg; 1.01 mmol) in DCM (10 mL) was added TFA (2 mL). The resulting mixture was stirred for 1 h at room temperature. The residue was diluted with water, then adjusted to pH 6-7 with sodium bicarbonate, then extracted with 3×40 mL of ethyl acetate. The combined organic layer was dried over anhydrous sodium sulfate and concentrated under vacuum to afford 2-methyl-5-({5H,6H,7H,8H-pyrido[3,4-d]pyrimidin-2-yl}amino)-2,3-dihydro-1H-isoindol-1-one as yellow solid (260 mg; 87.0%).

tert-butyl 8-methyl-7-{2-[(2-methyl-1-oxo-2,3-dihydro-1H-isoindol-5-yl)amino]-5H,6H,7H,8H-pyrido[3,4-d]pyrimidin-7-yl}-1H,2H,3H-pyrido[2,3-b][1,4]oxazine-1-carboxylate: To a stirred solution of 2-methyl-5-({5H,6H,7H,8H-pyrido[3,4-d]pyrimidin-2-yl}amino)-2,3-dihydro-1H-isoindol-1-one (200 mg; 0.68 mmol) and tert-butyl 7-bromo-8-methyl-1H,2H,3H-pyrido[2,3-b][1,4]oxazine-1-carboxylate (280 mg; 0.81 mmol) in DMF (10 mL) was added Pd-PEPPSI-IPentCl 2-methylpyridine (o-picoline) (60 mg; 0.07 mmol) and Cs₂CO₃ (470 mg; 1.37 mmol). The resulting mixture was stirred for 16 h at 90 degrees C. under nitrogen atmosphere. The mixture was concentrated under reduced pressure. The residue was purified by Prep-TLC (CH₂Cl₂/MeOH 19:1) to afford tert-butyl 8-methyl-7-{2-[(2-methyl-1-oxo-2,3-dihydro-1H-isoindol-5-yl)amino]-5H,6H,7H,8H-pyrido[3,4-d]pyrimidin-7-yl}-1H,2H,3H-pyrido[2,3-b][1,4]oxazine-1-carboxylate as yellow solid (80 mg; 21.7%).

2-methyl-5-[(7-{8-methyl-1H,2H,3H-pyrido[2,3-b][1,4]oxazin-7-yl}-5H,6H,7H,8H-pyrido[3,4-d]pyrimidin-2-yl)amino]-2,3-dihydro-1H-isoindol-1-one: To a stirred solution of tert-butyl 8-methyl-7-{2-[(2-methyl-1-oxo-2,3-dihydro-1H-isoindol-5-yl)amino]-5H,6H,7H,8H-pyrido[3,4-d]pyrimidin-7-yl}-1H,2H,3H-pyrido[2,3-b][1,4]oxazine-1-carboxylate (75 mg; 0.14 mmol) in DCM (10 mL) was added TFA (2 mL). The resulting mixture was stirred for 2 h at room temperature under nitrogen atmosphere. The residue was purified by Prep-HPLC with the following conditions: Column: XBridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 16% B to 46% B in 9 min., 46% B; Wave Length: 254 nm; RT1 (min.): 7; Number Of Runs: 1, to afford 2-methyl-5-[(7-{8-methyl-1H,2H,3H-pyrido[2,3-b][1,4]oxazin-7-yl}-5H,6H,7H,8H-pyrido[3,4-d]pyrimidin-2-yl)amino]-2,3-dihydro-1H-isoindol-1-one as white solid (7.30 mg; 11.9%).

HPLC 98.65%, 3.24 min. M/S: m/z=444.25. ¹H NMR (300 MHz, DMSO-d6) δ 9.95 (s, 1H), 8.42 (s, 1H), 8.11 (s, 1H), 7.77 (d, J=8.4 Hz, 1H), 7.54 (d, J=8.4 Hz, 1H), 7.31 (s, 1H), 5.54 (s, 1H), 4.42 (s, 2H), 4.20 (d, J=4.4 Hz, 2H), 4.01 (s, 2H), 3.14 (d, J=5.8 Hz, 2H), 3.04 (s, 3H), 2.84 (d, J=5.7 Hz, 2H), 2.06 (s, 3H).

### Example 97: Synthesis of Compound 97 7-(8-methyl-2,3-dihydro-1H-pyrido[2,3-b][1,4]oxazin-7-yl)-N-(4-(2-morpholinoethoxy)phenyl)-5,6,7,8-tetrahydropyrido[3,4-d]pyrimidin-2-amine

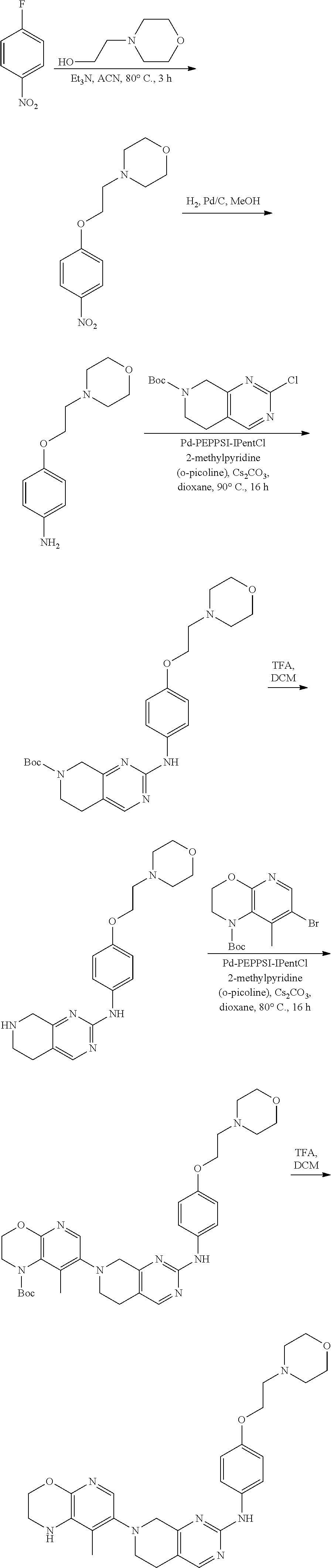

4-[2-(4-nitrophenoxy)ethyl]morpholine: To a stirred solution of 2-(morpholin-4-yl)ethan-1-ol (1 g; 7.250 mmol) and DMF (15 mL) was added sodium hydride (387 mg; 9.675 mmol) at 0 degrees C. under nitrogen atmosphere. The resulting mixture was stirred for 10 min. at 0 degrees C. under nitrogen atmosphere. After 10 min. 1-fluoro-4-nitrobenzene (718 mg; 4.834 mmol) was added to the above solution 0 degrees C. under nitrogen atmosphere. Then the resulting mixture was stirred for additional 2 h at 80 degrees C. The reaction was quenched by the addition of H₂O at room temperature and extracted by EA, the combined organic layers were washed with brine and dried over anhydrous Na₂SO₄. After filtration, the filtrate was concentrated under reduced pressure. The residue was purified by silica gel column chromatography and eluted with DCM:MeOH (10:1) to afford 4-[2-(4-nitrophenoxy)ethyl]morpholine (1 g; 78.9%) as yellow solid.

4-[2-(morpholin-4-yl)ethoxy]aniline: To a solution of 4-[2-(4-nitrophenoxy)ethyl]morpholine (450 mg; 1.716 mmol) in MeOH (50 mL) was added Palladium on activated carbon (100 mg; 0.177 mmol) in a round bottom flask. The mixture was hydrogenated at room temperature for 2 h under hydrogen atmosphere using a hydrogen balloon, filtered through a Celite pad and concentrated under reduced pressure to afford 4-[2-(morpholin-4-yl)ethoxy]aniline (422 mg; crude).

tert-butyl 2-({4-[2-(morpholin-4-yl)ethoxy]phenyl}amino)-5H,6H,7H,8H-pyrido[3,4-d]pyrimidine-7-carboxylate: To a solution of tert-butyl 2-chloro-5H,6H,7H,8H-pyrido[3,4-d]pyrimidine-7-carboxylate (497 mg; 1.843 mmol) and 4-[2-(morpholin-4-yl)ethoxy]aniline (421 mg; 1.841 mmol) in 1,4-dioxane (15 mL) were added Cs₂CO₃ (1264 mg; 3.685 mmol) and Pd-PEPPSI-IPentCl 2-methylpyridine (o-picoline) (163 mg; 0.184 mmol). The resulting mixture was stirred for 3 h at 100 degrees C. under nitrogen atmosphere. The resulting mixture was concentrated under reduced pressure. The residue was purified by silica gel column chromatography and eluted with DCM:MEOH (9:1) to afford tert-butyl 2-({4-[2-(morpholin-4-yl)ethoxy]phenyl}amino)-5H,6H,7H,8H-pyrido[3,4-d]pyrimidine-7-carboxylate (810 mg; 90.5%) as yellow solid.

N-{4-[2-(morpholin-4-yl)ethoxy]phenyl}-5H,6H,7H,8H-pyrido[3,4-d]pyrimidin-2-amine: To a stirred solution of tert-butyl 2-({4-[2-(morpholin-4-yl)ethoxy]phenyl}amino)-5H,6H,7H,8H-pyrido[3,4-d]pyrimidine-7-carboxylate (340 mg; 0.700 mmol) and DCM (15 mL) was added tribromoborane (0.209 mL; 2.101 mmol) at −80 degrees C. under nitrogen atmosphere. Then the resulting mixture was stirred for additional 2 h at −30 degrees C. Analytic after 2 h; test reaction: complete conversion. The mixture was basified to pH [7] with saturated NaHCO₃ (aq.). The resulting mixture was extracted with EtOAc. The combined organic layers were washed with NaCl (aq.) and dried over anhydrous Na₂SO₄. After filtration, the filtrate was concentrated under reduced pressure to afford N-{4-[2-(morpholin-4-yl)ethoxy]phenyl}-5H,6H,7H,8H-pyrido[3,4-d]pyrimidin-2-amine (367.00 mg; crude) as yellow solid.

tert-butyl 8-methyl-7-[2-({4-[2-(morpholin-4-yl)ethoxy]phenyl}amino)-5H,6H,7H,8H-pyrido[3,4-d]pyrimidin-7-yl]-1H,2H,3H-pyrido[2,3-b][1,4]oxazine-1-carboxylate: To a solution of N-{4-[2-(morpholin-4-yl)ethoxy]phenyl}-5H,6H,7H,8H-pyrido[3,4-d]pyrimidin-2-amine (95 mg; 0.263 mmol) and tert-butyl 7-bromo-8-methyl-1H,2H,3H-pyrido[2,3-b][1,4]oxazine-1-carboxylate (93 mg; 0.264 mmol) in 1,4-dioxane (8 mL) were added Cs₂CO₃ (181 mg; 0.528 mmol) and Pd-PEPPSI-IPentCl 2-methylpyridine (o-picoline) (23 mg; 0.026 mmol). The resulting mixture was stirred for 3 h at 100 degrees C. under nitrogen atmosphere. The resulting mixture was concentrated under reduced pressure. The residue was purified by silica gel column chromatography and eluted with DCM:MEOH (9:1) to afford tert-butyl 8-methyl-7-[2-({4-[2-(morpholin-4-yl)ethoxy]phenyl}amino)-5H,6H,7H,8H-pyrido[3,4-d]pyrimidin-7-yl]-1H,2H,3H-pyrido[2,3-b][1,4]oxazine-1-carboxylate (76 mg; 39.1%) as yellow solid.

7-{8-methyl-1H,2H,3H-pyrido[2,3-b][1,4]oxazin-7-yl}-N-{4-[2-(morpholin-4-yl)ethoxy]phenyl}-5H,6H,7H,8H-pyrido[3,4-d]pyrimidin-2-amine; formic acid: To a stirred solution of tert-butyl 8-methyl-7-[2-({4-[2-(morpholin-4-yl)ethoxy]phenyl}amino)-5H,6H,7H,8H-pyrido[3,4-d]pyrimidin-7-yl]-1H,2H,3H-pyrido[2,3-b][1,4]oxazine-1-carboxylate (75 mg; 0.102 mmol) and DCM (10 mL) was added tribromoborane (0.03 mL; 0.303 mmol) dropwise at −78 degrees C. under nitrogen atmosphere. The resulting mixture was stirred for 1 h at −30 degrees C. under nitrogen atmosphere. The mixture to pH [7] with conc saturated NaHCO₃ (aq.) and extracted by EA. The combined organic layers were washed with brine and dried over anhydrous Na₂SO₄. After filtration, the filtrate was concentrated under reduced pressure. The residue was purified by Prep-HPLC with the following conditions (Column: Xselect CSH OBD Column 30*150 mm, 5 μm, n; Mobile Phase A: water (0.1% FA), Mobile Phase B: ACN; Flow rate: 60 mL/min.; Gradient: 2% B to 15% B in 13 min., 15% B; Wave Length: 254/220 nm; RT1 (min.): 12.02; Number Of Runs: 2) to afford 7-{8-methyl-1H,2H,3H-pyrido[2,3-b][1,4]oxazin-7-yl}-N-{4-[2-(morpholin-4-yl)ethoxy]phenyl}-5H,6H,7H,8H-pyrido[3,4-d]pyrimidin-2-amine; formic acid (17.80 mg; 30.3%) as yellow solid.

HPLC 95.27%, 3.72 min. M/S: m/z=504.4. ¹H NMR (400 MHz, DMSO-d6) 9.28 (s, 1H), 8.27 (s, 1H), 7.61 (d, J=9.1 Hz, 2H), 7.27 (s, 1H), 6.85 (d, J=9.2 Hz, 2H), 5.51 (s, 1H), 4.19 (t, J=4.4 Hz, 2H), 4.03 (t, J=5.8 Hz, 2H), 3.92 (s, 2H), 3.63-3.52 (m, 4H), 3.33 (s, 2H), 3.11 (s, 2H), 2.77 (s, 2H), 2.66 (t, J=5.8 Hz, 2H), 2.46 (d, J=4.7 Hz, 4H), 2.03 (s, 3H).

### Example 98: Synthesis of Compound 98 N-(4-(2-methoxyethoxy)phenyl)-7-(8-methyl-2,3-dihydro-1H-pyrido[2,3-b][1,4]oxazin-7-yl)-5,6,7,8-tetrahydropyrido[3,4-d]pyrimidin-2-amine

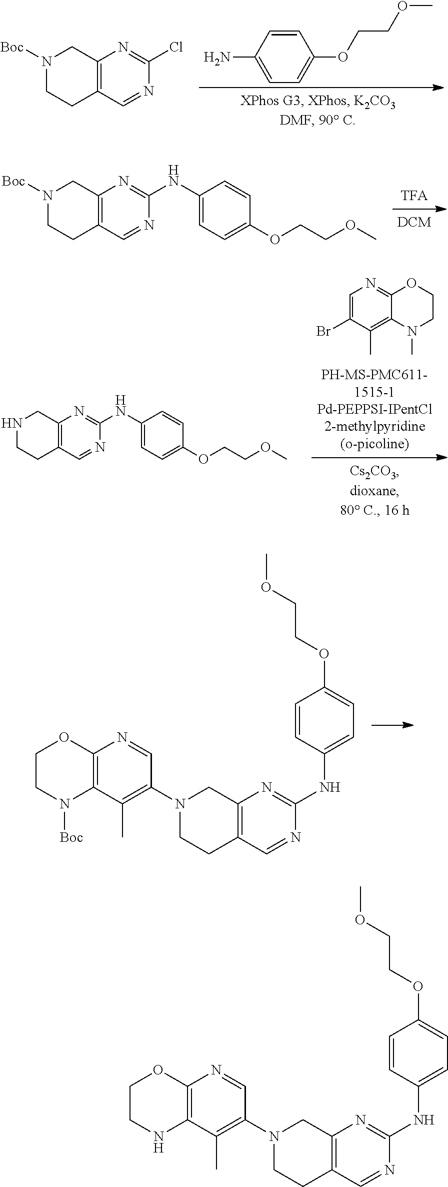

tert-butyl 2-{[4-(2-methoxyethoxy)phenyl]amino}-5H,6H,7H,8H-pyrido[3,4-d]pyrimidine-7-carboxylate: To a stirred solution of 4-(2-methoxyethoxy)aniline (400 mg; 2.27 mmol) and tert-butyl 2-chloro-5H,6H,7H,8H-pyrido[3,4-d]pyrimidine-7-carboxylate (730 mg; 2.71 mmol) in DMF (10.00 mL) was added Xphos Pd G3 (400 mg; 0.45 mmol), 2-dicyclohexyl p-2 minus 2-3 isopropyl biphenyl (220 mg; 0.44 mmol) and K₂CO₃ (660 mg; 4.54 mmol). The resulting mixture was stirred for 2 h at 90 degrees C. under nitrogen atmosphere. The mixture was concentrated under reduced pressure. The residue was purified by Prep-TLC (EA/PE 1:1) to afford tert-butyl 2-{[4-(2-methoxyethoxy)phenyl]amino}-5H,6H,7H,8H-pyrido[3,4-d]pyrimidine-7-carboxylate as yellow oil (800 mg; 87.9%).

N-[4-(2-methoxyethoxy)phenyl]-5H,6H,7H,8H-pyrido[3,4-d]pyrimidin-2-amine: To a stirred solution of tert-butyl 2-{[4-(2-methoxyethoxy)phenyl]amino}-5H,6H,7H,8H-pyrido[3,4-d]pyrimidine-7-carboxylate (400 mg; 1.00 mmol) in DCM (10 mL) was added TFA (2 mL). The resulting mixture was stirred for 2 h at room temperature. The mixture was diluted with water, then adjusted to pH 6-7 with sodium bicarbonate, and extracted with 3×40 mL of ethyl acetate. The combined organic layer was dried over anhydrous sodium sulfate and concentrated under vacuum to afford N-[4-(2-methoxyethoxy)phenyl]-5H,6H,7H,8H-pyrido[3,4-d]pyrimidin-2-amine as yellow oil (290 mg; crude).

tert-butyl 7-(2-{[4-(2-methoxyethoxy)phenyl]amino}-5H,6H,7H,8H-pyrido[3,4-d]pyrimidin-7-yl)-8-methyl-1H,2H,3H-pyrido[2,3-b][1,4]oxazine-1-carboxylate: To a stirred solution of N-[4-(2-methoxyethoxy)phenyl]-5H,6H,7H,8H-pyrido[3,4-d]pyrimidin-2-amine (200 mg; 0.67 mmol) and tert-butyl 7-bromo-8-methyl-1H,2H,3H-pyrido[2,3-b][1,4]oxazine-1-carboxylate (350 mg; 1.01 mmol) in DMF (10 mL) was added Pd-PEPPSI-IPentCl 2-methylpyridine (o-picoline) (60 mg; 0.07 mmol) and Cs₂CO₃ (460 mg; 1.34 mmol). The resulting mixture was stirred for 16 h at 90 degrees C. under nitrogen atmosphere. The mixture was concentrated under reduced pressure. The residue was purified by Prep-TLC (EA/PE 9:1) to afford tert-butyl 7-(2-{[4-(2-methoxyethoxy)phenyl]amino}-5H,6H,7H,8H-pyrido[3,4-d]pyrimidin-7-yl)-8-methyl-1H,2H,3H-pyrido[2,3-b][1,4]oxazine-1-carboxylate as yellow solid (200 mg; 54.7%).

N-[4-(2-methoxyethoxy)phenyl]-7-{8-methyl-1H,2H,3H-pyrido[2,3-b][1,4]oxazin-7-yl}-5H,6H,7H,8H-pyrido[3,4-d]pyrimidin-2-amine: To a stirred solution of tert-butyl 7-(2-{[4-(2-methoxyethoxy)phenyl]amino}-5H,6H,7H,8H-pyrido[3,4-d]pyrimidin-7-yl)-8-methyl-1H,2H,3H-pyrido[2,3-b][1,4]oxazine-1-carboxylate (100 mg; 0.18 mmol) in DCM (8 mL) was added BBr3 (50 mg; 0.19 mmol) at −30 degrees C. The resulting mixture was stirred for 2 h at −30 degrees C. under nitrogen atmosphere. The solvent was removed under vacuum. The residue was purified by Prep-HPLC with the following conditions: Column: XBridge Shield RP18 OBD Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 25% B to 55% B in 9 min., 55% B; Wave Length: 254 nm; RT1 (min.): 7; Number Of Runs: 1, to afford N-[4-(2-methoxyethoxy)phenyl]-7-{8-methyl-1H,2H,3H-pyrido[2,3-b][1,4]oxazin-7-yl}-5H,6H,7H,8H-pyrido[3,4-d]pyrimidin-2-amine as white solid (6.90 mg; 8.4%).

HPLC 99.64%, 3.63 min. M/S: m/z=449.25. ¹H NMR (300 MHz, DMSO-d6) δ 9.32 (s, 1H), 8.29 (s, 1H), 7.63 (d, J=8.5 Hz, 2H), 7.29 (s, 1H), 6.87 (d, J=8.3 Hz, 2H), 5.54 (s, 1H), 4.20 (s, 2H), 4.03 (d, J=5.9 Hz, 2H), 3.94 (s, 2H), 3.65 (s, 2H), 3.34 (s, 2H), 3.32 (s, 3H), 3.12 (s, 2H), 2.78 (s, 2H), 2.05 (s, 3H).

### Example 99: Synthesis of Compound 99 7-(8-methyl-2,3-dihydro-1H-pyrido[2,3-b][1,4]oxazin-7-yl)-N-(4-(morpholinomethyl)phenyl)-5,6,7,8-tetrahydropyrido[3,4-d]pyrimidin-2-amine

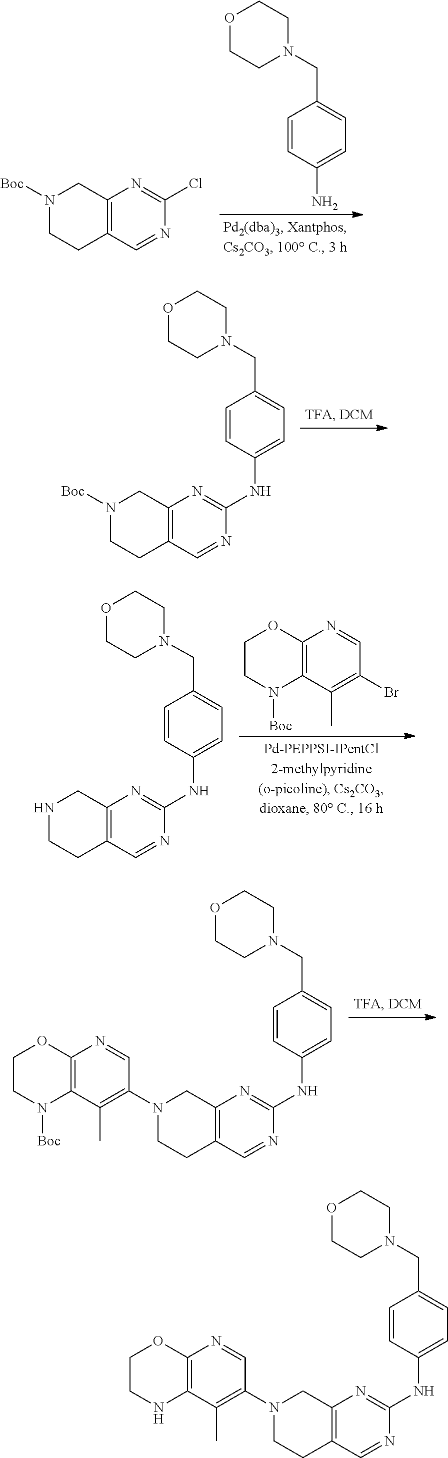

tert-butyl 2-({4-[(morpholin-4-yl)methyl]phenyl}amino)-5H,6H,7H,8H-pyrido[3,4-d]pyrimidine-7-carboxylate: To a solution of tert-butyl 2-chloro-5H,6H,7H,8H-pyrido[3,4-d]pyrimidine-7-carboxylate (500 mg; 1.854 mmol) and 4-[(morpholin-4-yl)methyl]aniline (563 mg; 2.782 mmol) in 1,4-dioxane (15 mL) were added Cs₂CO₃ (1272 mg; 3.709 mmol), tris(dibenzylideneacetone)dipalladium (189 mg; 0.186 mmol) and Xantphos (217 mg; 0.371 mmol). The resulting mixture was stirred for 3 h at 100 degrees C. under nitrogen atmosphere. The resulting mixture was concentrated under reduced pressure. The residue was purified by silica gel column chromatography and eluted with DCM:MEOH (9:1) to afford tert-butyl 2-({4-[(morpholin-4-yl)methyl]phenyl}amino)-5H,6H,7H,8H-pyrido[3,4-d]pyrimidine-7-carboxylate as yellow solid (880 mg; 103.9%).

N-{4-[(morpholin-4-yl)methyl]phenyl}-5H,6H,7H,8H-pyrido[3,4-d]pyrimidin-2-amine: To a stirred solution of tert-butyl 2-({4-[(morpholin-4-yl)methyl]phenyl}amino)-5H,6H,7H,8H-pyrido[3,4-d]pyrimidine-7-carboxylate (450 mg; 0.985 mmol) in DCM (15 mL) was added TFA (2 mL) dropwise at 0 degrees C. for 30 min. The reaction was quenched with H₂O at room temperature. The mixture was acidified to pH [7] with saturated NaHCO₃ (aq.). The resulting mixture was extracted with EtOAc. The combined organic layers were washed with NaCl (aq.) and dried over anhydrous Na₂SO₄. After filtration, the filtrate was concentrated under reduced pressure to purify to afford N-{4-[(morpholin-4-yl)methyl]phenyl}-5H,6H,7H,8H-pyrido[3,4-d]pyrimidin-2-amine as yellow solid (240 mg; 73.8%).

tert-butyl 8-methyl-7-[2-({4-[(morpholin-4-yl)methyl]phenyl}amino)-5H,6H,7H,8H-pyrido[3,4-d]pyrimidin-7-yl]-1H,2H,3H-pyrido[2,3-b][1,4]oxazine-1-carboxylate: To a solution of N-{4-[(morpholin-4-yl)methyl]phenyl}-5H,6H,7H,8H-pyrido[3,4-d]pyrimidin-2-amine (210 mg; 0.636 mmol) and tert-butyl 7-bromo-8-methyl-1H,2H,3H-pyrido[2,3-b][1,4]oxazine-1-carboxylate (448 mg; 1.273 mmol) in 1,4-dioxane (15 mL) were added Cs₂CO₃ (436 mg; 1.271 mmol) and Pd-PEPPSI-IPentCl 2-methylpyridine (o-picoline) (56 mg; 0.063 mmol). The resulting mixture was stirred for 3 h at 100 degrees C. under nitrogen atmosphere. The resulting mixture was concentrated under reduced pressure. The residue was purified by silica gel column chromatography and eluted with DCM:MEOH (9:1) to afford tert-butyl 8-methyl-7-[2-({4-[(morpholin-4-yl)methyl]phenyl}amino)-5H,6H,7H,8H-pyrido[3,4-d]pyrimidin-7-yl]-1H,2H,3H-pyrido[2,3-b][1,4]oxazine-1-carboxylate as yellow solid (350 mg; 89.6%).

7-{8-methyl-1H,2H,3H-pyrido[2,3-b][1,4]oxazin-7-yl}-N-{4-[(morpholin-4-yl)methyl]phenyl}-5H,6H,7H,8H-pyrido[3,4-d]pyrimidin-2-amine; formic acid: To a stirred solution of tert-butyl 8-methyl-7-[2-({4-[(morpholin-4-yl)methyl]phenyl}amino)-5H,6H,7H,8H-pyrido[3,4-d]pyrimidin-7-yl]-1H,2H,3H-pyrido[2,3-b][1,4]oxazine-1-carboxylate (100 mg; 0.163 mmol) in DCM (10 mL) was added TFA (3 mL) in portions at room temperature. The mixture was stirred for 3 hours at room temperature. The mixture was basified to pH [7] with saturated NaHCO₃ (aq.). The resulting mixture was extracted with EtOAc. The combined organic layers were washed with NaCl (aq.) and dried over anhydrous Na₂SO₄. Ater filtration, the filtrate was concentrated under reduced pressure. The residue was purified by Prep-HPLC with the following conditions (Column: XSelect CSH Prep C18 OBD Column, 19*250 mm, 5 μm; Mobile Phase A: water (0.05% FA), Mobile Phase B: ACN; Flow rate: 20 mL/min; Gradient: 20% B to 50% B in 8 min., 50% B; Wave Length: 254 nm; RTT (min.): 6.8; Number Of Runs: 2) to afford 7-{8-methyl-1H,2H,3H-pyrido[2,3-b][1,4]oxazin-7-yl}-N-{4-[(morpholin-4-yl)methyl]phenyl}-5H,6H,7H,8H-pyrido[3,4-d]pyrimidin-2-amine; formic acid as light yellow solid (22.9 mg; 27.0%).

HPLC 99.81%, 2.73 min. M/S: m/z=474.25. ¹H NMR (400 MHz, DMSO-d6) δ 9.49 (s, 1H), 8.32 (s, 1H), 8.14 (s, 1H), 7.80-7.64 (m, 2H), 7.28 (s, 1H), 7.22-7.14 (m, 2H), 5.51 (t, J=2.7 Hz, 1H), 4.19 (t, J=4.4 Hz, 2H), 3.95 (s, 2H), 3.57 (t, J=4.6 Hz, 4H), 3.44 (s, 2H), 3.30 (d, J=6.1 Hz, 2H), 3.12 (t, J=5.6 Hz, 2H), 2.79 (t, J=5.7 Hz, 2H), 2.39 (s, 2H), 2.04 (s, 3H).

### Example 100: Synthesis of Compound 100 7-(8-methyl-2,3-dihydro-1H-pyrido[2,3-b][1,4]oxazin-7-yl)-N-(4-(1-methylpiperidin-4-yl)phenyl)-5,6,7,8-tetrahydropyrido[3,4-d]pyrimidin-2-amine

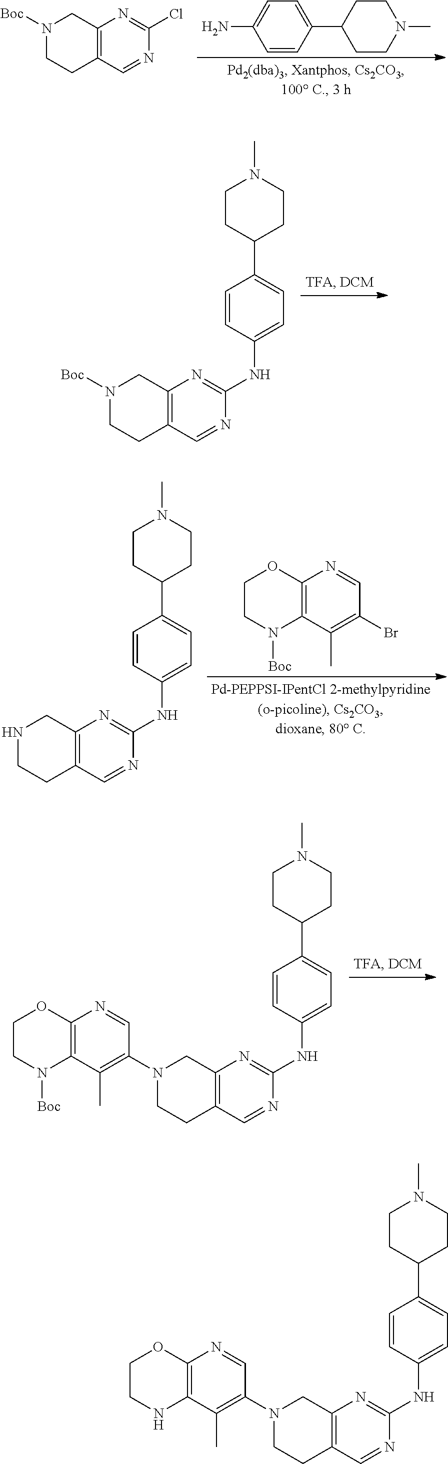

tert-butyl 2-{[4-(1-methylpiperidin-4-yl)phenyl]amino}-5H,6H,7H,8H-pyrido[3,4-d]pyrimidine-7-carboxylate: To a solution of tert-butyl 2-chloro-5H,6H,7H,8H-pyrido[3,4-d]pyrimidine-7-carboxylate (500 mg; 1.854 mmol) and 4-(1-methylpiperidin-4-yl)aniline (557 mg; 2.781 mmol) in 1,4-dioxane (15 mL) were added Cs₂CO₃ (1272 mg; 3.709 mmol), tris(dibenzylideneacetone)dipalladium (189 mg; 0.186 mmol) and Xantphos (217 mg; 0.371 mmol). The resulting mixture was stirred for 3 h at 100 degrees C. under nitrogen atmosphere. The resulting mixture was concentrated under reduced pressure. The residue was purified by silica gel column chromatography and eluted with DCM:MEOH (9:1) to afford tert-butyl 2-{[4-(1-methylpiperidin-4-yl)phenyl]amino}-5H,6H,7H,8H-pyrido[3,4-d]pyrimidine-7-carboxylate as yellow solid (500 mg; 62.6%).

N-[4-(1-methylpiperidin-4-yl)phenyl]-5H,6H,7H,8H-pyrido[3,4-d]pyrimidin-2-amine: To a stirred solution of tert-butyl 2-{[4-(1-methylpiperidin-4-yl)phenyl]amino}-5H,6H,7H,8H-pyrido[3,4-d]pyrimidine-7-carboxylate (500 mg; 1.579 mmol) in DCM (15 mL) was added TFA (2 mL) dropwise at 0 degrees C. for 30 min. The reaction was quenched with H₂O at room temperature, the mixture was acidified to pH [7] with saturated NaHCO₃ (aq.). The resulting mixture was extracted with EtOAc. The combined organic layers were washed with NaCl (aq.) and dried over anhydrous Na₂SO₄. After filtration, the filtrate was concentrated under reduced pressure to purify by C18 to afford N-[4-(1-methylpiperidin-4-yl)phenyl]-5H,6H,7H,8H-pyrido[3,4-d]pyrimidin-2-amine as yellow solid (210 mg; 40.4%).

tert-butyl 8-methyl-7-(2-{[4-(1-methylpiperidin-4-yl)phenyl]amino}-5H,6H,7H,8H-pyrido[3,4-d]pyrimidin-7-yl)-1H,2H,3H-pyrido[2,3-b][1,4]oxazine-1-carboxylate: To a solution of N-[4-(1-methylpiperidin-4-yl)phenyl]-5H,6H,7H,8H-pyrido[3,4-d]pyrimidin-2-amine (200 mg; 0.607 mmol) and tert-butyl 7-bromo-8-methyl-1H,2H,3H-pyrido[2,3-b][1,4]oxazine-1-carboxylate (427 mg; 1.213 mmol) in 1,4-dioxane (15 mL) were added Cs₂CO₃ (416 mg; 1.213 mmol) and Pd-PEPPSI-IPentCl 2-methylpyridine (o-picoline) (54 mg; 0.061 mmol). The resulting mixture was stirred for 3 h at 90 degrees C. under nitrogen atmosphere. The resulting mixture was concentrated under reduced pressure. The residue was purified by silica gel column chromatography and eluted with DCM:MEOH (9:1) to afford tert-butyl 8-methyl-7-(2-{[4-(1-methylpiperidin-4-yl)phenyl]amino}-5H,6H,7H,8H-pyrido[3,4-d]pyrimidin-7-yl)-1H,2H,3H-pyrido[2,3-b][1,4]oxazine-1-carboxylate as yellow solid (300 mg; 78.2%).

7-{8-methyl-1H,2H,3H-pyrido[2,3-b][1,4]oxazin-7-yl}-N-[4-(1-methylpiperidin-4-yl)phenyl]-5H,6H,7H,8H-pyrido[3,4-d]pyrimidin-2-amine; formic acid: To a stirred solution of tert-butyl 8-methyl-7-(2-{[4-(1-methylpiperidin-4-yl)phenyl]amino}-5H,6H,7H,8H-pyrido[3,4-d]pyrimidin-7-yl)-1H,2H,3H-pyrido[2,3-b][1,4]oxazine-1-carboxylate (200.000 mg; 0.316 mmol; 1.0 eq.) in DCM (10.000 mL) was added tribromoborane (0.094 mL; 0.948 mmol; 3.0 eq.) in portions at −80 degrees C. The resulting mixture was stirred for 1 h at −30 degrees C. under argon atmosphere. The mixture was basified to pH [7] with saturated NaHCO₃ (aq.). The resulting mixture was extracted with EtOAc. The combined organic layers were washed with NaCl (aq.) and dried over anhydrous Na₂SO₄. After filtration, the filtrate was concentrated under reduced pressure. The residue was purified by Prep-HPLC with the following conditions (Column: Xselect CSH OBD Column 30*150 mm, 5 μm, n; Mobile Phase A: water (0.1% FA), Mobile Phase B: ACN; Flow rate: 60 mL/min.; Gradient: 2% B to 15% B in 13 min., 15% B; Wave Length: 254/220 nm; RTT (min.): 12.02; Number Of Runs: 2) to afford 7-{8-methyl-1H,2H,3H-pyrido[2,3-b][1,4]oxazin-7-yl}-N-[4-(1-methylpiperidin-4-yl)phenyl]-5H,6H,7H,8H-pyrido[3,4-d]pyrimidin-2-amine; formic acid as light yellow solid (23.20 mg; 14.0%).

HPLC:98.61% purity, 2.75 min. MS: m/z=472.30 [M+H]⁺. ¹H NMR (400 MHz, DMSO-d6) δ 9.40 (s, 1H), 8.30 (s, 1H), 8.21 (s, 1H), 7.71-7.56 (m, 2H), 7.28 (s, 1H), 7.15-7.04 (m, 2H), 5.51 (s, 1H), 4.20-4.18 (m, 2H), 3.94 (s, 2H), 3.35-3.25 (m, 2H), 3.11 (t, J=5.6 Hz, 2H), 2.95 (d, J=11.1 Hz, 2H), 2.78 (t, J=5.6 Hz, 2H), 2.46-2.36 (m, 1H), 2.28 (s, 3H), 2.18-2.07 (m, 2H), 2.04 (s, 3H), 1.75-1.61 (m, 4H).

### Example 101: Synthesis of Compound 101 7-(1,8-dimethyl-2,3-dihydro-1H-pyrido[2,3-b][1,4]oxazin-7-yl)-N-(6-methyl-5,6,7,8-tetrahydro-1,6-naphthyridin-3-yl)-5,6,7,8-tetrahydropyrido[3,4-d]pyrimidin-2-amine

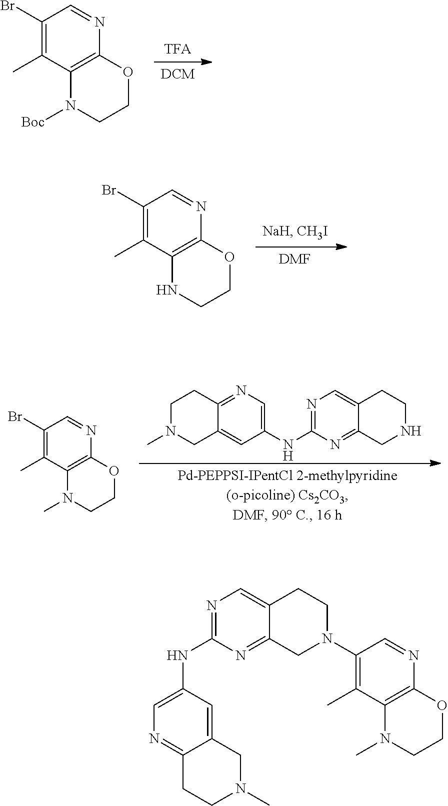

7-bromo-8-methyl-1H,2H,3H-pyrido[2,3-b][1,4]oxazine: To a stirred solution of tert-butyl 7-bromo-8-methyl-1H,2H,3H-pyrido[2,3-b][1,4]oxazine-1-carboxylate (400 mg; 1.22 mmol) in DCM (5 mL) was added TFA (2 mL). The resulting mixture was stirred for 2 h at room temperature under nitrogen atmosphere. The resulting solution was extracted with 3×40 mL of ethyl acetate. The organic layers were combined, dried, and concentrated under vacuum to afford 7-bromo-8-methyl-1H,2H,3H-pyrido[2,3-b][1,4]oxazine as white solid (250 mg; Crude Product).

7-bromo-1,8-dimethyl-1H,2H,3H-pyrido[2,3-b][1,4]oxazine: To a stirred solution of 7-bromo-8-methyl-1H,2H,3H-pyrido[2,3-b][1,4]oxazine (240 mg; 1.05 mmol) in DMF (10 mL) was added NaH (80 mg; 2.00 mmol). The above mixture was stirred for 30 min. at 0 degrees C. MeI (0.22 mL; 3.35 mmol) was added. The resulting mixture was stirred for 2 h at room temperature under nitrogen atmosphere. The reaction was then quenched by the addition of 30 mL of water, then extracted with DCM. The organic layers were combined, dried and concentrated under vacuum to afford 7-bromo-1,8-dimethyl-1H,2H,3H-pyrido[2,3-b][1,4]oxazine as yellow solid (220 mg; Crude Product).

N-(7-{1,8-dimethyl-1H,2H,3H-pyrido[2,3-b][1,4]oxazin-7-yl}-5H,6H,7H,8H-pyrido[3,4-d]pyrimidin-2-yl)-6-methyl-5,6,7,8-tetrahydro-1,6-naphthyridin-3-amine: To a stirred solution of 6-methyl-N-{5H,6H,7H,8H-pyrido[3,4-d]pyrimidin-2-yl}-5,6,7,8-tetrahydro-1,6-naphthyridin-3-amine (250 mg; 0.80 mmol) and 7-bromo-1,8-dimethyl-1H,2H,3H-pyrido[2,3-b][1,4]oxazine (210 mg; 0.86 mmol) in DMF (10 mL) was added Pd-PEPPSI-IPentCl 2-methylpyridine (o-picoline) (100 mg; 0.11 mmol) and Cs₂CO₃ (900 mg; 2.62 mmol). The resulting mixture was stirred for 12 h at 90 degrees C. under nitrogen atmosphere. The residue was purified by Prep-HPLC with the following conditions (Column: XBridge Shield RP18 OBD Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 20% B to 50% B in 9 min., 50% B; Wave Length: 254 nm; RT1 (min.): 7; Number Of Runs: 1) to afford N-(7-{1,8-dimethyl-1H,2H,3H-pyrido[2,3-b][1,4]oxazin-7-yl}-5H,6H,7H,8H-pyrido[3,4-d]pyrimidin-2-yl)-6-methyl-5,6,7,8-tetrahydro-1,6-naphthyridin-3-amine as white solid (4.40 mg; 1.1%).

HPLC 99.60% purity, 2.61 min. MS: m/z=459.20 [M+H]⁺. ¹H NMR (300 MHz, DMSO-d6) | δ 9.57 (s, 1H), 8.64 (d, J=2.3 Hz, 1H), 8.36 (s, 1H), 7.88 (d, J=2.4 Hz, 1H), 7.67 (s, 1H), 4.25 (dd, J=5.1, 3.5 Hz, 2H), 4.04 (s, 2H), 3.50 (s, 2H), 3.19 (t, J=5.7 Hz, 2H), 3.10-3.03 (m, 2H), 2.83 (t, J=5.5 Hz, 4H), 2.70 (d, J=8.5 Hz, 5H), 2.36 (s, 3H), 2.26 (s, 3H).

### Example 102: Synthesis of Compound 102 1-(5-((7-(8-methyl-2,3-dihydro-1H-pyrido[2,3-b][1,4]oxazin-7-yl)-5,6,7,8-tetrahydropyrido[3,4-d]pyrimidin-2-yl)amino)pyridin-2-yl)piperidin-4-ol

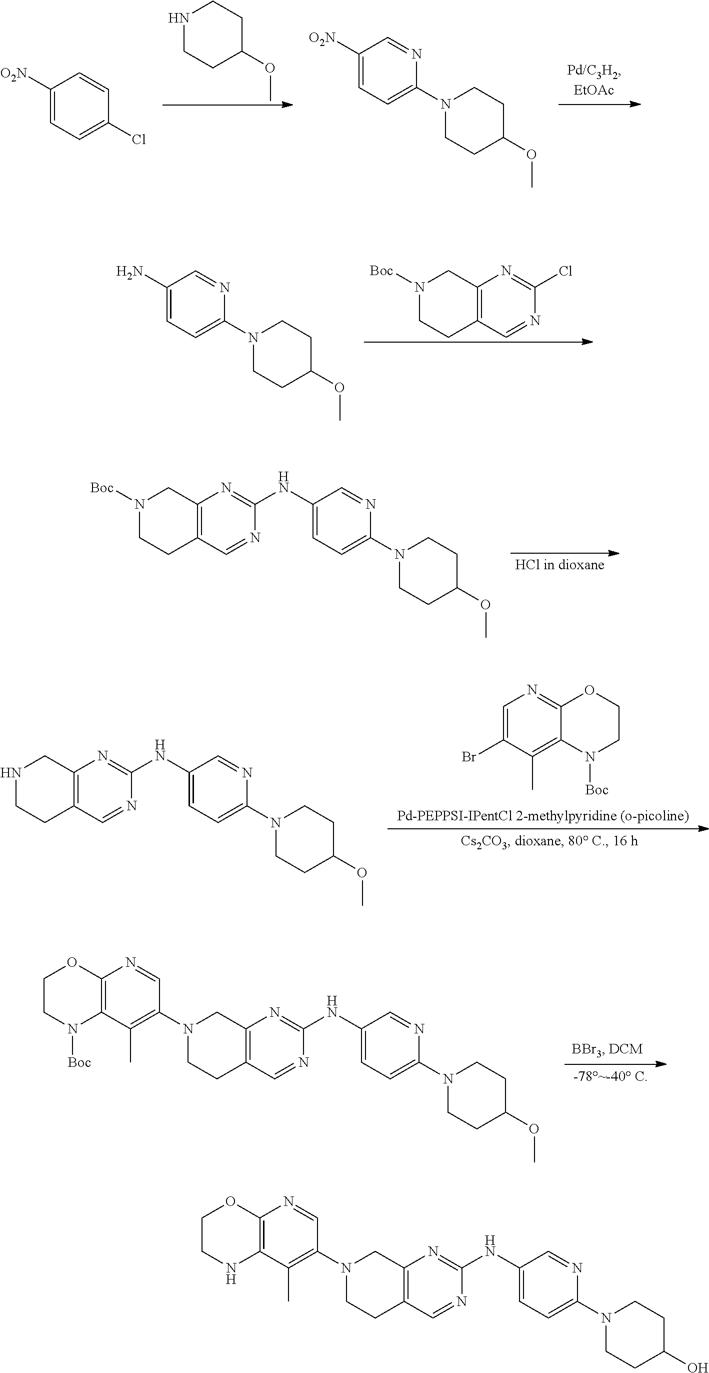

2-(4-methoxypiperidin-1-yl)-5-nitropyridine: To a stirred solution of 2-chloro-5-nitropyridine (1 g; 6.31 mmol) and 4-methoxypiperidine (1.1 g; 9.07 mmol) in DMF (10 mL) was added K₂CO₃ (2.70 g; 18.56 mmol). The resulting mixture was stirred for 2 h at 50 degrees C. The reaction was quenched by water, the resulting mixture was extracted with EtOAc (3×30 mL). The combined organic layers were washed with brine and dried over anhydrous Na₂SO₄. After filtration, the filtrate was concentrated under reduced pressure. The residue was purified by silica gel column chromatography and eluted with PE/EtOAc (5:1) to afford 2-(4-methoxypiperidin-1-yl)-5-nitropyridine as yellow solid (1.2 g; 80.2%).

6-(4-methoxypiperidin-1-yl)pyridin-3-amine: To a solution of 2-(4-methoxypiperidin-1-yl)-5-nitropyridine (1.2 g; 5.06 mmol) in AcOEt (25 mL) was added Pd/C (200 mg; 0.47 mmol) under nitrogen atmosphere in a 50 mL round-bottom flask. The mixture was hydrogenated at room temperature for 1 h under hydrogen atmosphere using a hydrogen balloon. The resulting mixture was filtered and the filter cake was washed with EtOAc (3×10 mL). The filtrate was concentrated under reduced pressure. This resulted in 6-(4-methoxypiperidin-1-yl)pyridin-3-amine as brown solid (1 g; Crude Product).

tert-butyl 2-{[6-(4-methoxypiperidin-1-yl)pyridin-3-yl]amino}-5H,6H,7H,8H-pyrido[3,4-d]pyrimidine-7-carboxylate: To a stirred solution of 6-(4-methoxypiperidin-1-yl)pyridin-3-amine (500.00 mg; 2.41 mmol) and tert-butyl 2-chloro-5H,6H,7H,8H-pyrido[3,4-d]pyrimidine-7-carboxylate (400 mg; 1.48 mmol) in DMF (10 mL) was added K₂CO₃ (800 mg; 5.51 mmol) Xphos (180 mg; 0.36 mmol) and XPhos Pd G3 (160 mg; 0.18 mmol). The resulting mixture was stirred for 3 h at 90 degrees C. The resulting mixture was concentrated under vacuum. The residue was purified by silica gel column chromatography and eluted with PE/EtOAc (1:1) to afford it. This resulted in tert-butyl 2-{[6-(4-methoxypiperidin-1-yl)pyridin-3-yl]amino}-5H,6H,7H,8H-pyrido[3,4-d]pyrimidine-7-carboxylate as brown solid (450 mg; 42.3%).

6-(4-methoxypiperidin-1-yl)-N-{5H,6H,7H,8H-pyrido[3,4-d]pyrimidin-2-yl}pyridin-3-amine: To a stirred solution of tert-butyl 2-{[6-(4-methoxypiperidin-1-yl)pyridin-3-yl]amino}-5H,6H,7H,8H-pyrido[3,4-d]pyrimidine-7-carboxylate (450 mg; 1.02 mmol) in 1,4-dioxane (10 mL) was added HCl (gas) in 1,4-dioxane (3 mL). The resulting mixture was stirred for 2 h at room temperature. The resulting mixture was concentrated under reduced pressure. This resulted in 6-(4-methoxypiperidin-1-yl)-N-{5H,6H,7H,8H-pyrido[3,4-d]pyrimidin-2-yl}pyridin-3-amine as yellow oil (350 mg; Crude Product).

tert-butyl 7-(2-{[6-(4-methoxypiperidin-1-yl)pyridin-3-yl]amino}-5H,6H,7H,8H-pyrido[3,4-d]pyrimidin-7-yl)-8-methyl-1H,2H,3H-pyrido[2,3-b][1,4]oxazine-1-carboxylate: To a stirred solution of 6-(4-methoxypiperidin-1-yl)-N-{5H,6H,7H,8H-pyrido[3,4-d]pyrimidin-2-yl}pyridin-3-amine (350 mg) and tert-butyl 7-bromo-8-methyl-1H,2H,3H-pyrido[2,3-b][1,4]oxazine-1-carboxylate (500 mg; 1.50 mmol) in 1,4-dioxane (15 mL) was added Cs₂CO₃ (1.20 g; 3.50 mmol) and Pd-PEPPSI-IPentCl 2-methylpyridine (o-picoline) (88 mg; 0.10 mmol) under nitrogen atmosphere. The resulting mixture was stirred for overnight at 90 degrees C. under nitrogen atmosphere. The resulting mixture was concentrated under vacuum. The residue was purified by silica gel column chromatography and eluted with CH₂Cl₂/MeOH (1:1). This resulted in tert-butyl 7-(2-{[6-(4-methoxypiperidin-1-yl)pyridin-3-yl]amino}-5H,6H,7H,8H-pyrido[3,4-d]pyrimidin-7-yl)-8-methyl-1H,2H,3H-pyrido[2,3-b][1,4]oxazine-1-carboxylate as yellow solid (300 mg; 49.6%).

1-{5-[(7-{8-methyl-1H,2H,3H-pyrido[2,3-b][1,4]oxazin-7-yl}-5H,6H,7H,8H-pyrido[3,4-d]pyrimidin-2-yl)amino]pyridin-2-yl}piperidin-4-ol; formic acid: To a stirred solution of tert-butyl 7-(2-{[6-(4-methoxypiperidin-1-yl)pyridin-3-yl]amino}-5H,6H,7H,8H-pyrido[3,4-d]pyrimidin-7-yl)-8-methyl-1H,2H,3H-pyrido[2,3-b][1,4]oxazine-1-carboxylate (170 mg; 0.29 mmol) in DCM (10 mL) was added BBr₃ in DCM (1.00 mL; 1.00 mmol) dropwise at −78 degrees C. under nitrogen atmosphere. The resulting mixture was stirred for 2 h at −30 degrees C. under nitrogen atmosphere. The resulting mixture was concentrated under vacuum. The residue was purified by reverse flash chromatography. This resulted in 1-{5-[(7-{8-methyl-1H,2H,3H-pyrido[2,3-b][1,4]oxazin-7-yl}-5H,6H,7H,8H-pyrido[3,4-d]pyrimidin-2-yl)amino]pyridin-2-yl}piperidin-4-ol; formic acid as off-white solid (12.90 mg; 8.29%).

HPLC 96.65% purity, 2.55 min. MS: m/z=475.20 [M+H]⁺. ¹H NMR (300 MHz, DMSO-d6, ppm): 8.37 (d, J=2.7 Hz, 1H), 8.24 (s, 1H), 7.81 (dd, J=9.1, 2.8 Hz, 1H), 7.25 (s, 1H), 6.80 (d, J=9.2 Hz, 1H), 4.19 (dd, J=5.2, 3.4 Hz, 2H), 3.98-3.84 (m, 4H), 3.72-3.64 (m, 1H), 3.34-3.23 (m, 2H), 3.15-3.05 (m, 2H), 3.04-2.89 (m, 2H), 2.77 (d, J=5.6 Hz, 2H), 2.03 (s, 3H), 1.85-1.72 (m, 2H), 1.45-1.27 (m, 2H).

### Example 103: Synthesis of Compound 103 7-(8-methyl-2,3-dihydro-1H-pyrido[2,3-b][1,4]oxazin-7-yl)-N-(4-(4-(trifluoromethoxy)piperidin-1-yl)phenyl)-5,6,7,8-tetrahydropyrido[3,4-d]pyrimidin-2-amine

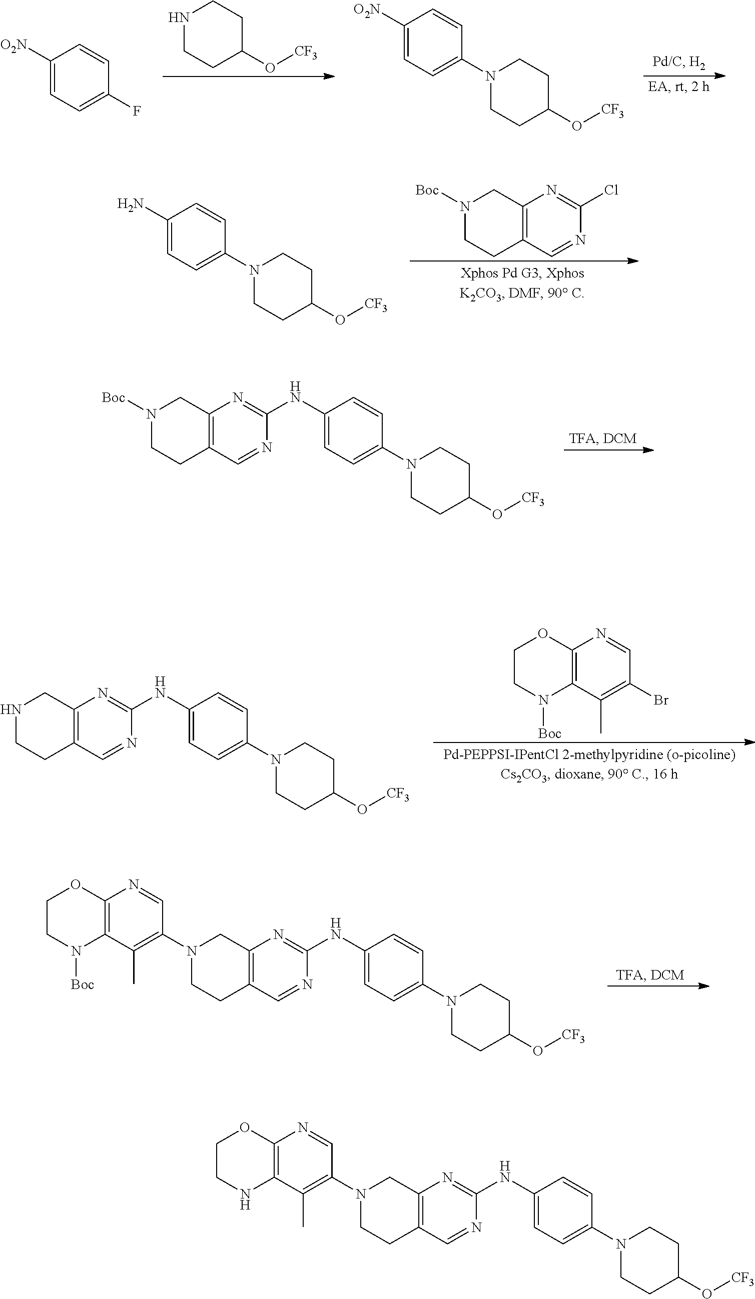

1-(4-nitrophenyl)-4-(trifluoromethoxy)piperidine: To a stirred solution of 1-fluoro-4-nitrobenzene (573 mg; 3.86 mmol) and 4-(trifluoromethoxy)piperidine (1000 mg; 5.62 mmol) in DMF (20 mL) was added K₂CO₃ (1600 mg; 11.02 mmol) at room temperature. The resulting mixture was stirred for overnight at 80 degrees C. The resulting mixture was concentrated under vacuum. The residue was purified by silica gel column chromatography eluted with PE/EA (10:1) to afford. This resulted in 1-(4-nitrophenyl)-4-(trifluoromethoxy)piperidine as yellow solid (1000 mg; 89.3%).

4-[4-(trifluoromethoxy)piperidin-1-yl]aniline: To a solution of 1-(4-nitrophenyl)-4-(trifluoromethoxy)piperidine (700 mg; 2.41 mmol) in AcOEt (15 mL) was added Pd/C (200 mg; 0.47 mmol) under nitrogen atmosphere in a 50 mL round-bottom. The mixture was hydrogenated at room temperature for 3 h under hydrogen atmosphere using a hydrogen balloon, filtered through a Celite pad and concentrated under reduced pressure. The resulting mixture was concentrated under reduced pressure. This resulted in 4-[4-(trifluoromethoxy)piperidin-1-yl]aniline as red oil (600 mg; Crude Product).

tert-butyl 2-({4-[4-(trifluoromethoxy)piperidin-1-yl]phenyl}amino)-5H,6H,7H,8H-pyrido[3,4-d]pyrimidine-7-carboxylate: To a stirred solution of 4-[4-(trifluoromethoxy)piperidin-1-yl]aniline (582 mg; 2.24 mmol) and tert-butyl 2-chloro-5H,6H,7H,8H-pyrido[3,4-d]pyrimidine-7-carboxylate (500 mg; 1.85 mmol) in DMF (10 mL) was added XPhos Pd G3 (314.50 mg; 0.35 mmol), Xphos (174 mg; 0.35 mmol) and K₂CO₃ (513.40 mg; 3.54 mmol) at room temperature under nitrogen atmosphere. The resulting mixture was stirred for 2 h at 90 degrees C. under nitrogen atmosphere. The resulting mixture was concentrated under vacuum. The residue was purified by silica gel column chromatography eluted with PE/EA (3:1) to afford tert-butyl 2-({4-[4-(trifluoromethoxy)piperidin-1-yl]phenyl}amino)-5H,6H,7H,8H-pyrido[3,4-d]pyrimidine-7-carboxylate as brown oil (780 mg; 61.7%).

N-{4-[4-(trifluoromethoxy)piperidin-1-yl]phenyl}-5H,6H,7H,8H-pyrido[3,4-d]pyrimidin-2-amine: To a stirred solution of tert-butyl 2-({4-[4-(trifluoromethoxy)piperidin-1-yl]phenyl}amino)-5H,6H,7H,8H-pyrido[3,4-d]pyrimidine-7-carboxylate (100 mg; 0.18 mmol) in DCM (5 mL) was added TFA (0.50 mL) at room temperature. The resulting mixture was stirred for 2 h at room temperature. The resulting mixture was concentrated under reduced pressure.

This resulted in N-{4-[4-(trifluoromethoxy)piperidin-1-yl]phenyl}-5H,6H,7H,8H-pyrido[3,4-d]pyrimidin-2-amine as brown solid (70 mg; Crude Product).

tert-butyl 8-methyl-7-[2-({4-[4-(trifluoromethoxy)piperidin-1-yl]phenyl}amino)-5H,6H,7H,8H-pyrido[3,4-d]pyrimidin-7-yl]-1H,2H,3H-pyrido[2,3-b][1,4]oxazine-1-carboxylate: To a stirred solution of N-{4-[4-(trifluoromethoxy)piperidin-1-yl]phenyl}-5H,6H,7H,8H-pyrido[3,4-d]pyrimidin-2-amine (70 mg; 0.18 mmol) and tert-butyl 7-bromo-8-methyl-1H,2H,3H-pyrido[2,3-b][1,4]oxazine-1-carboxylate (100 mg; 0.30 mmol) in DMF (5 mL) was added Pd-PEPPSI-IPentCl 2-methylpyridine (o-picoline) (30 mg; 0.03 mmol) and Cs₂CO₃ (300 mg; 0.87 mmol) at room temperature under nitrogen atmosphere. The resulting mixture was stirred for overnight at 90 degrees C. under nitrogen atmosphere. The resulting mixture was concentrated under vacuum. The residue was purified by silica gel column chromatography eluted with DCM/MeOH (15:1) to afford tert-butyl 8-methyl-7-[2-({4-[4-(trifluoromethoxy)piperidin-1-yl]phenyl}amino)-5H,6H,7H,8H-pyrido[3,4-d]pyrimidin-7-yl]-1H,2H,3H-pyrido[2,3-b][1,4]oxazine-1-carboxylate as brown solid (60 mg; 47.2%).

7-{8-methyl-1H,2H,3H-pyrido[2,3-b][1,4]oxazin-7-yl}-N-{4-[4-(trifluoromethoxy)piperidin-1-yl]phenyl}-5H,6H,7H,8H-pyrido[3,4-d]pyrimidin-2-amine hydrochloride: To a stirred solution of tert-butyl 8-methyl-7-[2-({4-[4-(trifluoromethoxy)piperidin-1-yl]phenyl}amino)-5H,6H,7H,8H-pyrido[3,4-d]pyrimidin-7-yl]-1H,2H,3H-pyrido[2,3-b][1,4]oxazine-1-carboxylate (50 mg; 0.07 mmol) in DCM (5 mL) was added TFA (1 mL) at room temperature. The resulting mixture was stirred for 2 h at room temperature. The resulting mixture was concentrated under reduced pressure. The crude product was purified by Prep-HPLC with the following conditions: Column: XBridge Prep OBD C18 Column, 30×150 mm, 5 μm; Mobile Phase A: water (0.05% HCl), Mobile Phase B:ACN; Flow rate:60 m/min.; Gradient:35 B to 55 B in 8 min. This resulted in 7-{8-methyl-1H,2H,3H-pyrido[2,3-b][1,4]oxazin-7-yl}-N-{4-[4-(trifluoromethoxy)piperidin-1-yl]phenyl}-5H,6H,7H,8H-pyrido[3,4-d]pyrimidin-2-amine hydrochloride as orange solid (5.90 mg; 13.8%).

HPLC 96.81% purity, 3.77 min. MS: m/z=542.20 [M+H]⁺. ¹H NMR (300 MHz, DMSO-d6) ppm 8.40 (s, 1H), 7.94-7.85 (m, 2H), 7.70-7.61 (m, 2H), 7.39 (s, 1H), 4.85 (dd, J=8.5, 4.5 Hz, 1H), 4.51 (t, J=4.5 Hz, 2H), 4.07 (s, 2H), 3.64 (s, 4H), 3.48 (d, J=4.6 Hz, 2H), 3.16 (d, J=6.0 Hz, 2H), 2.86 (s, 2H), 2.35 (s, 2H), 2.31-2.21 (m, 2H), 2.21 (s, 3H).

### Example 104: Synthesis of Compound 104 7-(8-methyl-2,3-dihydro-1H-pyrido[2,3-b][1,4]oxazin-7-yl)-N-(2-methylisoindolin-5-yl)-5,6,7,8-tetrahydropyrido[3,4-d]pyrimidin-2-amine

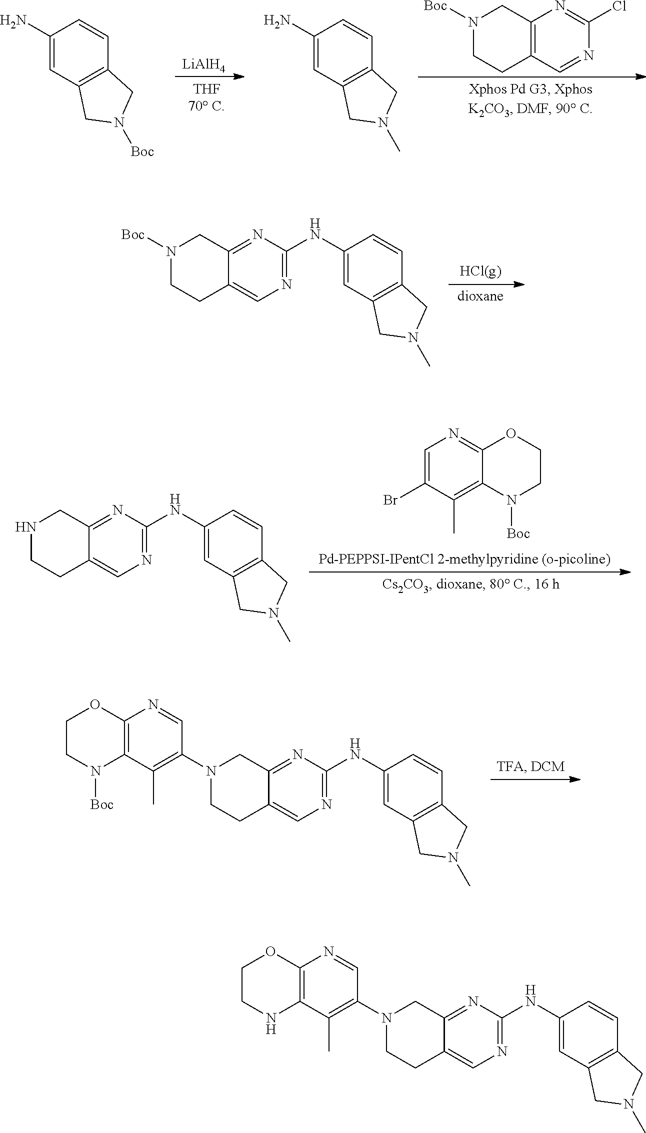

2-methyl-2,3-dihydro-1H-isoindol-5-amine: To a stirred solution of tert-butyl 5-amino-2,3-dihydro-1H-isoindole-2-carboxylate (1 g; 4.14 mmol) in THF (20 mL) was added LiAlH₄ (1.5 g; 37.55 mmol) at 0 degrees C. The resulting mixture was stirred for 5 h at 70 degrees C. under nitrogen atmosphere. The resulting mixture was quenched by the addition of 1 g of Na₂SO₄*10H₂O at 0 degrees C. The resulting solution was stirred for 0.5 h, then collected by filtration and washed with DCM. The resulting mixture was concentrated under reduced pressure. This resulted in 2-methyl-2,3-dihydro-1H-isoindol-5-amine as yellow solid (433 mg; Crude Product).

tert-butyl 2-[(2-methyl-2,3-dihydro-1H-isoindol-5-yl)amino]-5H,6H,7H,8H-pyrido[3,4-d]pyrimidine-7-carboxylate: To a stirred solution of 2-methyl-2,3-dihydro-1H-isoindol-5-amine (300 mg; 1.66 mmol) and tert-butyl 2-chloro-5H,6H,7H,8H-pyrido[3,4-d]pyrimidine-7-carboxylate (453.6 mg; 1.68 mmol) in DMF (10.00 mL) was added XPhos Pd G3 (290.50 mg; 0.33 mmol), Xphos (160.20 mg; 0.32 mmol) and K₂CO₃ (700 mg; 4.81 mmol) at room temperature under nitrogen atmosphere. The resulting mixture was stirred for 2 h at 90 degrees C. under nitrogen atmosphere. The resulting mixture was concentrated under vacuum. The residue was purified by silica gel column chromatography eluted with DCM/MeOH (15:1) to afford tert-butyl 2-[(2-methyl-2,3-dihydro-1H-isoindol-5-yl)amino]-5H,6H,7H,8H-pyrido[3,4-d]pyrimidine-7-carboxylate as brown solid (303 mg; 44%).

2-methyl-N-{5H,6H,7H,8H-pyrido[3,4-d]pyrimidin-2-yl}-2,3-dihydro-1H-isoindol-5-amine: To a stirred solution of tert-butyl 2-[(2-methyl-2,3-dihydro-1H-isoindol-5-yl)amino]-5H,6H,7H,8H-pyrido[3,4-d]pyrimidine-7-carboxylate (303 mg; 0.75 mmol) in 1,4-dioxane (5 mL) was added HCl (gas) in 1,4-dioxane (3 mL; 12.00 mmol) at room temperature. The resulting mixture was stirred for 2 h at room temperature. The crude product was obtained under reduced pressure. This resulted in 2-methyl-N-{5H,6H,7H,8H-pyrido[3,4-d]pyrimidin-2-yl}-2,3-dihydro-1H-isoindol-5-amine (243.00 mg; Crude Product).

tert-butyl 8-methyl-7-{2-[(2-methyl-2,3-dihydro-1H-isoindol-5-yl)amino]-5H,6H,7H,8H-pyrido[3,4-d]pyrimidin-7-yl}-1H,2H,3H-pyrido[2,3-b][1,4]oxazine-1-carboxylate: To a stirred solution of 2-methyl-N-{5H,6H,7H,8H-pyrido[3,4-d]pyrimidin-2-yl}-2,3-dihydro-TH-isoindol-5-amine (243 mg; 0.91 mmol) and tert-butyl 7-bromo-8-methyl-1H,2H,3H-pyrido[2,3-b][1,4]oxazine-1-carboxylate (420 mg; 1.28 mmol) in DMF (20 mL) was added Pd-PEPPSI-IPentCl 2-methylpyridine (o-picoline) (200 mg; 0.23 mmol) and Cs₂CO₃ (1300 mg; 3.79 mmol) at room temperature under nitrogen atmosphere. The resulting mixture was stirred for overnight at 90 degrees C. under nitrogen atmosphere. The resulting mixture was concentrated under vacuum. The residue was purified by silica gel column chromatography eluted with DCM/MeOH (1:1) to afford tert-butyl 8-methyl-7-{2-[(2-methyl-2,3-dihydro-TH-isoindol-5-yl)amino]-5H,6H,7H,8H-pyrido[3,4-d]pyrimidin-7-yl}-1H,2H,3H-pyrido[2,3-b][1,4]oxazine-1-carboxylate as brown solid (185 mg; 38.2%).

2-methyl-N-(7-{8-methyl-1H,2H,3H-pyrido[2,3-b][1,4]oxazin-7-yl}-5H,6H,7H,8H-pyrido[3,4-d]pyrimidin-2-yl)-2,3-dihydro-1H-isoindol-5-amine hydrochloride: To a stirred solution of tert-butyl 2-[(2-methyl-2,3-dihydro-1H-isoindol-5-yl)amino]-5H,6H,7H,8H-pyrido[3,4-d]pyrimidine-7-carboxylate (180 mg; 0.05 mmol) in DCM (1 mL) was added BBr₃ (0.05 mL; 0.99 mmol) at −60 degrees C. under nitrogen atmosphere. The resulting mixture was stirred for 2 h at −30 degrees C. under nitrogen atmosphere. The resulting mixture was quenched by the addition of 10 mL of NH₄Cl. The resulting solution was extracted with 3×10 mL of dichloromethane, and the organic layers combined, washing with 3×10 mL brine, drying over Na₂SO₄ and concentrated under vacuum. The crude product was purified by Prep-HPLC with the following conditions: Column: XBridge Prep OBD C18 Column, 30×150 mm, 5 μm; Mobile Phase A: water (0.05% HCl), Mobile Phase B:ACN; Flow rate:60 mL/min.; Gradient:5 B to 30 B in 8 min; RT1: 6.8 min. This resulted in 2-methyl-N-(7-{8-methyl-1H,2H,3H-pyrido[2,3-b][1,4]oxazin-7-yl}-5H,6H,7H,8H-pyrido[3,4-d]pyrimidin-2-yl)-2,3-dihydro-1H-isoindol-5-amine hydrochloride as brown yellow solid (4.00 mg; 16.9%).

HPLC 96.86% purity, 3.21 min. MS: m/z=430.20 [M+H]⁺. ¹H NMR (300 MHz, DMSO-d6) ppm 8.39 (s, 1H), 7.86 (s, 1H), 7.69 (d, J=8.5 Hz, 1H), 7.41 (s, 1H), 7.30 (d, J=8.4 Hz, 1H), 4.74 (m, J=13.3 Hz, 2H), 4.51 (d, J=5.4 Hz, 2H), 4.48-4.31 (m, 2H), 4.06 (s, 2H), 3.61 (d, J=11.3 Hz, 2H), 3.18 (s, 2H), 3.01 (d, J=2.3 Hz, 3H), 2.86 (s, 2H), 2.21 (s, 3H).

### Example 105: Synthesis of Compound 105 N-(6-(2,2-difluoroethyl)-5,6,7,8-tetrahydro-1,6-naphthyridin-3-yl)-7-(8-methyl-2,3-dihydro-1H-pyrido[2,3-b][1,4]oxazin-7-yl)-5,6,7,8-tetrahydropyrido[3,4-d]pyrimidin-2-amine

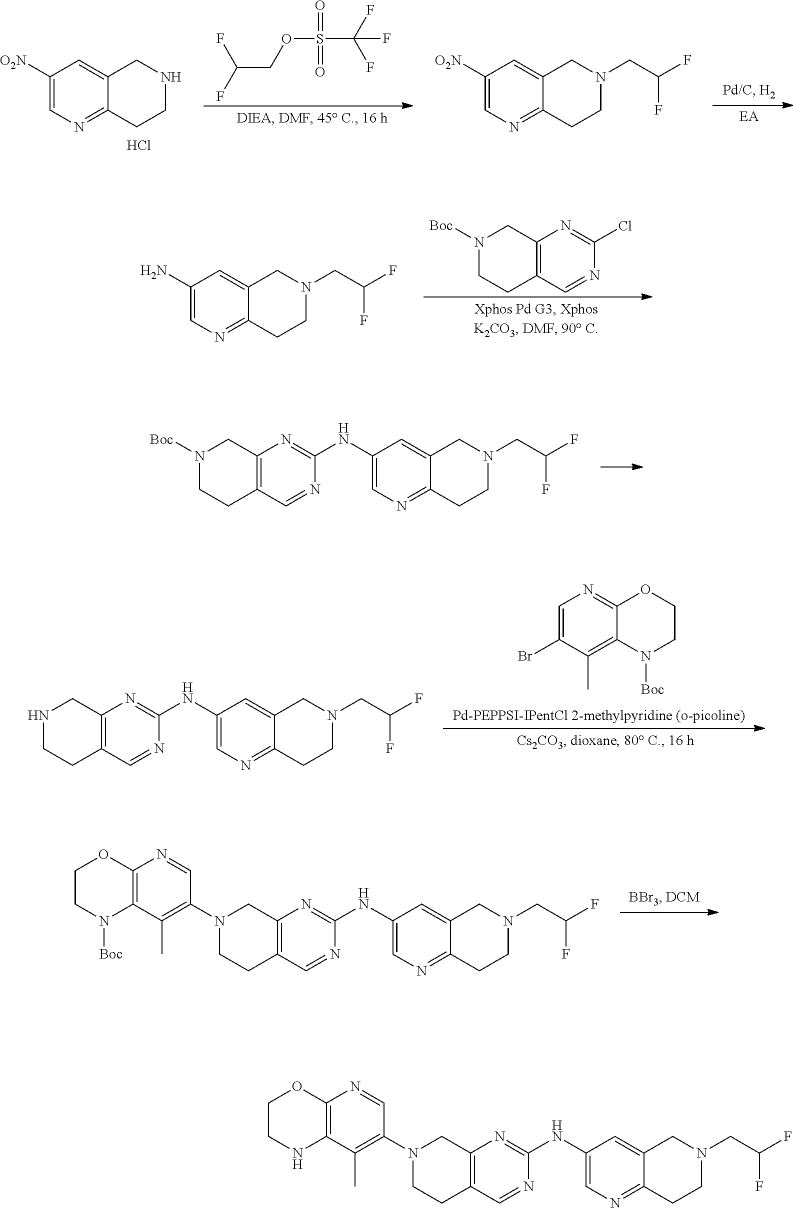

6-(2,2-difluoroethyl)-3-nitro-5,6,7,8-tetrahydro-1,6-naphthyridine: To a stirred solution of 3-nitro-5,6,7,8-tetrahydro-1,6-naphthyridine hydrochloride (1 g; 4.64 mmol) and 2,2-difluoroethyl trifluoromethanesulfonate (1.20 g; 5.60 mmol) in DMF (20 mL) was added DIEA (1.40 g; 10.29 mmol). The resulting mixture was stirred for overnight at 80 degrees C. The resulting mixture was concentrated under vacuum. The residue was purified by silica gel column chromatography and eluted with CH₂Cl₂/MeOH (50:1) to afford 6-(2,2-difluoroethyl)-3-nitro-5,6,7,8-tetrahydro-1,6-naphthyridine as brown solid (450 mg; 39.9%).

6-(2,2-difluoroethyl)-5,6,7,8-tetrahydro-1,6-naphthyridin-3-amine: To a solution of 6-(2,2-difluoroethyl)-3-nitro-5,6,7,8-tetrahydro-1,6-naphthyridine (440 mg; 1.81 mmol) in AcOEt (20 mL) was added Pd/C (100 mg; 0.09 mmol) under nitrogen atmosphere in a 50 mL round-bottom flask. The mixture was hydrogenated at room temperature for 1 h under hydrogen atmosphere using a hydrogen balloon. The resulting mixture was filtered; the filter cake was washed with EtOAc (3×10 mL). The filtrate was concentrated under reduced pressure. This resulted in 6-(2,2-difluoroethyl)-5,6,7,8-tetrahydro-1,6-naphthyridin-3-amine as brown oil (350 mg; Crude Product).

tert-butyl 2-{[6-(2,2-difluoroethyl)-5,6,7,8-tetrahydro-1,6-naphthyridin-3-yl]amino}-5H,6H,7H,8H-pyrido[3,4-d]pyrimidine-7-carboxylate: To a stirred solution of 6-(2,2-difluoroethyl)-5,6,7,8-tetrahydro-1,6-naphthyridin-3-amine (350 mg; 1.88 mmol) and tert-butyl 2-chloro-5H,6H,7H,8H-pyrido[3,4-d]pyrimidine-7-carboxylate (550 mg; 2.04 mmol) in DMF (10 mL) was added K₂CO₃ (800 mg; 5.50 mmol) Xphos (200 mg; 0.40 mmol) and XPhos Pd G3 (180 mg; 0.20 mmol) under nitrogen atmosphere. The resulting mixture was stirred for 2 h at 90 degrees C. under nitrogen atmosphere. The resulting mixture was extracted with EtOAc (3×30 mL). The combined organic layers were washed with EtOAc (3×10 mL) and dried over anhydrous Na₂SO₄. After filtration, the filtrate was concentrated under reduced pressure. The residue was purified by silica gel column chromatography and eluted with PE/EtOAc (1:5) to afford tert-butyl 2-{[6-(2,2-difluoroethyl)-5,6,7,8-tetrahydro-1,6-naphthyridin-3-yl]amino}-5H,6H,7H,8H-pyrido[3,4-d]pyrimidine-7-carboxylate as brown solid (450 mg; 51.1%).

6-(2,2-difluoroethyl)-N-{5H,6H,7H,8H-pyrido[3,4-d]pyrimidin-2-yl}-5,6,7,8-tetrahydro-1,6-naphthyridin-3-amine: To a stirred solution of tert-butyl 2-{[6-(2,2-difluoroethyl)-5,6,7,8-tetrahy dro-1,6-naphthyridin-3-yl]amino}-5H,6H,7H,8H-pyrido[3,4-d]pyrimidine-7-carboxylate (400 mg; 0.85 mmol) in 1,4-dioxane (10 mL) was added HCl (gas) in 1,4-dioxane (2 mL; 8.00 mmol) dropwise at 25 degrees C. The resulting mixture was stirred for 2 h at room temperature under nitrogen atmosphere. The resulting mixture was concentrated under vacuum. This resulted in 6-(2,2-difluoroethyl)-N-{5H,6H,7H,8H-pyrido[3,4-d]pyrimidin-2-yl}-5,6,7,8-tetrahydro-1,6-naphthyridin-3-amine as brown solid (300 mg; Crude Product).

tert-butyl 7-(2-{[6-(2,2-difluoroethyl)-5,6,7,8-tetrahydro-1,6-naphthyridin-3-yl]amino}-5H,6H,7H,8H-pyrido[3,4-d]pyrimidin-7-yl)-8-methyl-1H,2H,3H-pyrido[2,3-b][1,4]oxazine-1-carboxylate: To a stirred solution of 6-(2,2-difluoroethyl)-N-{5H,6H,7H,8H-pyrido[3,4-d]pyrimidin-2-yl}-5,6,7,8-tetrahydro-1,6-naphthyridin-3-amine (150 mg; 0.36 mmol) and tert-butyl 7-bromo-8-methyl-1H,2H,3H-pyrido[2,3-b][1,4]oxazine-1-carboxylate (240 mg; 0.73 mmol) in 1,4-dioxane (10 mL) was added Cs₂CO₃ (450 mg; 1.31 mmol) and Pd-PEPPSI-IPentCl 2-methylpyridine (o-picoline) (50 mg; 0.06 mmol). The resulting mixture was stirred for overnight at 90 degrees C. under nitrogen atmosphere. The resulting mixture was concentrated under vacuum. The residue was purified by silica gel column chromatography and eluted with CH₂Cl₂/MeOH (1:1) to afford it. This resulted in tert-butyl 7-(2-{[6-(2,2-difluoroethyl)-5,6,7,8-tetrahydro-1,6-naphthyridin-3-yl]amino}-5H,6H,7H,8H-pyrido[3,4-d]pyrimidin-7-yl)-8-methyl-1H,2H,3H-pyrido[2,3-b][1,4]oxazine-1-carboxylate as yellow solid (130 mg; 60.5%).

6-(2,2-difluoroethyl)-N-(7-{8-methyl-1H,2H,3H-pyrido[2,3-b][1,4]oxazin-7-yl}-5H,6H,7H,8H-pyrido[3,4-d]pyrimidin-2-yl)-5,6,7,8-tetrahydro-1,6-naphthyridin-3-amine: To a stirred solution of tert-butyl 7-(2-{[6-(2,2-difluoroethyl)-5,6,7,8-tetrahydro-1,6-naphthyridin-3-yl]amino}-5H,6H,7H,8H-pyrido[3,4-d]pyrimidin-7-yl)-8-methyl-1H,2H,3H-pyrido[2,3-b][1,4]oxazine-1-carboxylate (120 mg; 0.20 mmol) in DCM (25 mL) was BBr3 in DCM (0.50 mL; 0.50 mmol) dropwise at −78 degrees C. under nitrogen atmosphere. The resulting mixture was stirred for 2 h at −60 degrees C. under nitrogen atmosphere. The reaction was quenched by the addition of sat. NaHCO₃ (aq.) (50 mL) at room temperature. The resulting mixture was extracted with EtOAc (3×30 mL). The combined organic layers were washed with EtOAc (3×10 mL) and dried over anhydrous Na₂SO₄. After filtration, the filtrate was concentrated under reduced pressure. The residue was purified by reverse flash chromatography with the following conditions: Column: XBridge Prep OBD C18 Column, 30×150 mm, 5 μm; Mobile Phase A: water (10 mmol/L NH₄HCO₃+0.05% NH₄OH), Mobile Phase B: ACN; Flow rate:60 mL/min.; Gradient: 20 B to 50 B in 8 min., RT1:6.6). This resulted in 6-(2,2-difluoroethyl)-N-(7-{8-methyl-1H,2H,3H-pyrido[2,3-b][1,4]oxazin-7-yl}-5H,6H,7H,8H-pyrido[3,4-d]pyrimidin-2-yl)-5,6,7,8-tetrahydro-1,6-naphthyridin-3-amine as white solid (12.60 mg; 11.7%).

HPLC 93.01% purity, 3.73 min. MS: m/z=495.25 [M+H]⁺. ¹H NMR (300 MHz, DMSO-d6, ppm): 9.63 (s, 1H), 8.65 (d, J=2.4 Hz, 1H), 8.36 (s, 1H), 7.92 (d, J=2.5 Hz, 1H), 7.30 (s, 1H), 6.57-5.95 (m, 1H), 5.54 (d, J=3.2 Hz, 1H), 4.25-4.16 (m, 2H), 3.97 (s, 2H), 3.75 (s, 2H), 3.32 (s, 2H), 3.13 (d, J=5.8 Hz, 2H), 2.96-2.76 (m, 8H), 2.06 (s, 3H).

### Example 106: Synthesis of Compound 106 7-(8-methyl-2,3-dihydro-1H-pyrido[2,3-b][1,4]oxazin-7-yl)-N-(6-(2,2,2-trifluoroethyl)-5,6,7,8-tetrahydro-1,6-naphthyridin-3-yl)-5,6,7,8-tetrahydropyrido[3,4-d]pyrimidin-2-amine

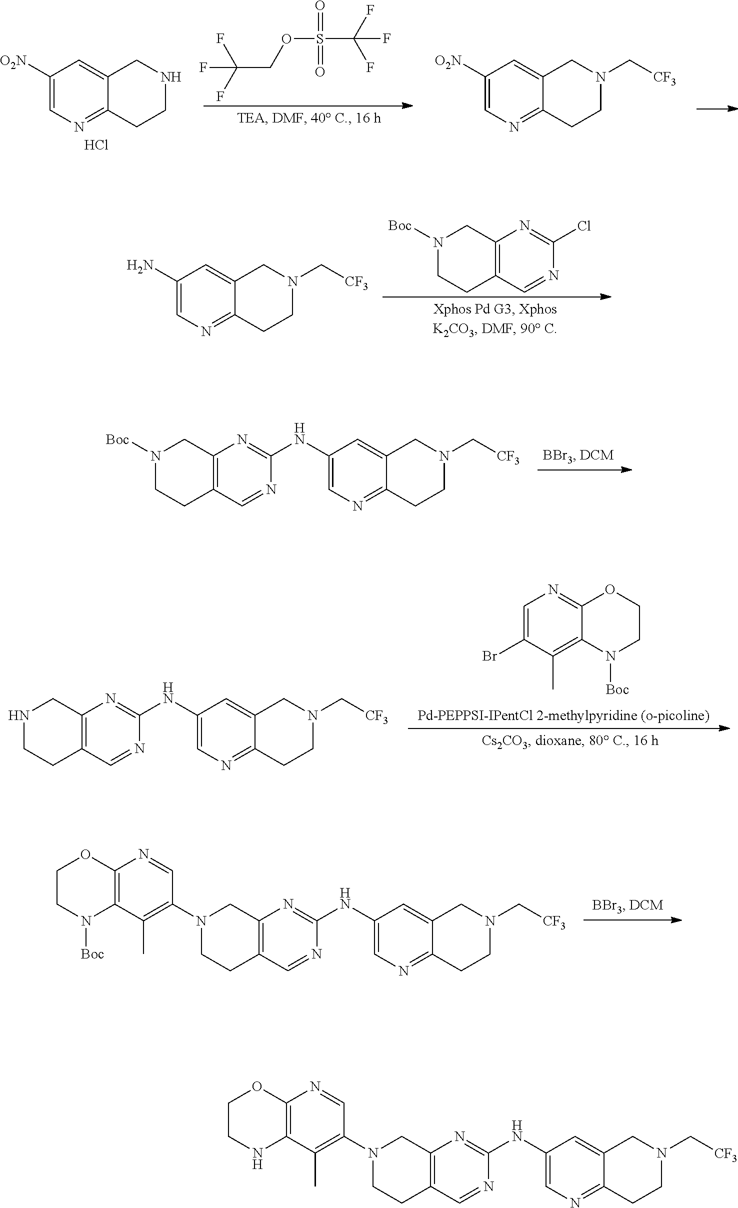

3-nitro-6-(2,2,2-trifluoroethyl)-5,6,7,8-tetrahydro-1,6-naphthyridine: To a stirred solution of 3-nitro-5,6,7,8-tetrahydro-1,6-naphthyridine hydrochloride (1.0 g; 4.64 mmol) and 2,2,2-trifluoroethyl trifluoromethanesulfonate (2.0 g; 8.62 mmol) in DMF (20 mL) was Et3N (1.65 mL; 11.74 mmol). The resulting mixture was stirred for overnight at 40 degrees C. The resulting mixture was extracted with EtOAc (3×30 mL). The combined organic layers were washed with EtOAc (3×10 mL) and dried over anhydrous Na₂SO₄. After filtration, the filtrate was concentrated under reduced pressure. The residue was purified by silica gel column chromatography and eluted with CH₂Cl₂/MeOH (15:1) to afford it. This resulted in 3-nitro-6-(2,2,2-trifluoroethyl)-5,6,7,8-tetrahydro-1,6-naphthyridine as brown solid (900 mg; 74.3%).

6-(2,2,2-trifluoroethyl)-5,6,7,8-tetrahydro-1,6-naphthyridin-3-amine: To a solution of 3-nitro-6-(2,2,2-trifluoroethyl)-5,6,7,8-tetrahydro-1,6-naphthyridine (900 mg; 3.45 mmol) in AcOEt (15 mL) was added Pd/C (200 mg; 0.19 mmol) under nitrogen atmosphere in a 50 mL round-bottom flask. The mixture was hydrogenated at room temperature for 1 h under hydrogen atmosphere using a hydrogen balloon. The resulting mixture was concentrated under vacuum to afford 6-(2,2,2-trifluoroethyl)-5,6,7,8-tetrahydro-1,6-naphthyridin-3-amine as yellow solid (600 mg; Crude Product).

tert-butyl 2-{[6-(2,2,2-trifluoroethyl)-5,6,7,8-tetrahydro-1,6-naphthyridin-3-yl]amino}-5H,6H,7H,8H-pyrido[3,4-d]pyrimidine-7-carboxylate: To a stirred solution of 6-(2,2,2-trifluoroethyl)-5,6,7,8-tetrahydro-1,6-naphthyridin-3-amine (400 mg; 1.73 mmol) and tert-butyl 2-chloro-5H,6H,7H,8H-pyrido[3,4-d]pyrimidine-7-carboxylate (500 mg; 1.85 mmol) in DMF (10.00 mL) was added K₂CO₃ (710 mg; 4.88 mmol) Xphos (170 mg; 0.34 mmol) and XPhos Pd G3 (150 mg; 0.17 mmol) under nitrogen atmosphere. The resulting mixture was stirred for 2 h at 90 degrees C. under nitrogen atmosphere. The reaction was quenched by water, the resulting mixture was extracted with EtOAc (3×30 mL). The combined organic layers were washed with brine and dried over anhydrous Na₂SO₄. After filtration, the filtrate was concentrated under reduced pressure. The residue was purified by silica gel column chromatography and eluted with PE/EtOAc CH2Cl2/MeOH (1:1) to afford tert-butyl 2-{[6-(2,2,2-trifluoroethyl)-5,6,7,8-tetrahydro-1,6-naphthyridin-3-yl]amino}-5H,6H,7H,8H-pyrido[3,4-d]pyrimidine-7-carboxylate as yellow solid (600 mg, 73.8%).

N-{5H,6H,7H,8H-pyrido[3,4-d]pyrimidin-2-yl}-6-(2,2,2-trifluoroethyl)-5,6,7,8-tetrahydro-1,6-naphthyridin-3-amine: To a stirred solution of tert-butyl 2-{[6-(2,2,2-trifluoroethyl)-5,6,7,8-tetrahydro-1,6-naphthyridin-3-yl]amino}-5H,6H,7H,8H-pyrido[3,4-d]pyrimidine-7-carboxylate (580 mg; 1.23 mmol) in DCM (30 mL) was added BBr₃ in DCM (2.50 mL; 2.50 mmol) dropwise at −78 degrees C. The resulting mixture was stirred for 2 h at −78 degrees C. The reaction was quenched by the addition of sat. NaHCO₃ (aq.) (50 mL) at room temperature. The resulting mixture was extracted with EtOAc (3×30 mL). The combined organic layers were washed with brine and dried over anhydrous Na₂SO₄. After filtration, the filtrate was concentrated under reduced pressure. This resulted in N-{5H,6H,7H,8H-pyrido[3,4-d]pyrimidin-2-yl}-6-(2,2,2-trifluoroethyl)-5,6,7,8-tetrahydro-1,6-naphthyridin-3-amine as yellow solid (480 mg; Crude Product).

tert-butyl 8-methyl-7-(2-{[6-(2,2,2-trifluoroethyl)-5,6,7,8-tetrahydro-1,6-naphthyridin-3-yl]amino}-5H,6H,7H,8H-pyrido[3,4-d]pyrimidin-7-yl)-1H,2H,3H-pyrido[2,3-b][1,4]oxazine-1-carboxylate: To a stirred solution of N-{5H,6H,7H,8H-pyrido[3,4-d]pyrimidin-2-yl}-6-(2,2,2-trifluoroethyl)-5,6,7,8-tetrahydro-1,6-naphthyridin-3-amine (350 mg; 0.74 mmol) and tert-butyl 7-bromo-8-methyl-1H,2H,3H-pyrido[2,3-b][1,4]oxazine-1-carboxylate (360 mg; 1.09 mmol) in 1,4-dioxane (10 mL) was added Cs₂CO₃ (1 g; 2.92 mmol) and Pd-PEPPSI-IPentCl 2-methylpyridine (o-picoline) (100 mg; 0.11 mmol) under nitrogen atmosphere. The resulting mixture was stirred for overnight at 90 degrees C. under nitrogen atmosphere. The resulting mixture was concentrated under vacuum. The residue was purified by silica gel column chromatography and eluted with CH₂Cl₂/MeOH (5:1) to afford it. This resulted in tert-butyl 8-methyl-7-(2-{[6-(2,2,2-trifluoroethyl)-5,6,7,8-tetrahydro-1,6-naphthyridin-3-yl]amino}-5H,6H,7H,8H-pyrido[3,4-d]pyrimidin-7-yl)-1H,2H,3H-pyrido[2,3-b][1,4]oxazine-1-carboxylate as yellow solid (330 mg; 58.5%).

N-(7-{8-methyl-1H,2H,3H-pyrido[2,3-b][1,4]oxazin-7-yl}-5H,6H,7H,8H-pyrido[3,4-d]pyrimidin-2-yl)-6-(2,2,2-trifluoroethyl)-5,6,7,8-tetrahydro-1,6-naphthyridin-3-amine: To a stirred solution of tert-butyl 8-methyl-7-(2-{[6-(2,2,2-trifluoroethyl)-5,6,7,8-tetrahydro-1,6-naphthyridin-3-yl]amino}-5H,6H,7H,8H-pyrido[3,4-d]pyrimidin-7-yl)-1H,2H,3H-pyrido[2,3-b][1,4]oxazine-1-carboxylate (300 mg; 0.40 mmol) in DCM (25 mL) was added BBr3 in DCM (1.20 mL; 1.20 mmol) dropwise at −78 degrees C. under nitrogen atmosphere. The resulting mixture was stirred for 2 h at −60 degrees C. under nitrogen atmosphere. The reaction was quenched by the addition of sat. NaHCO₃ (aq.) (50 mL) at room temperature. The resulting mixture was extracted with EtOAc (3×30 mL). The combined organic layers were washed with brine and dried over anhydrous Na₂SO₄. After filtration, the filtrate was concentrated under reduced pressure. The residue was purified by reverse flash chromatography with the following conditions: Column: XBridge Prep OBD C18 Column, 19*250 mm, 5 μm; Mobile Phase A: water (10 mmol/L NH₄HCO₃), Mobile Phase B: ACN; Flow rate: 20 mL/min.; Gradient: 35 B to 46 B in 10 min, RT1: 8). This resulted in N-(7-{8-methyl-1H,2H,3H-pyrido[2,3-b][1,4]oxazin-7-yl}-5H,6H,7H,8H-pyrido[3,4-d]pyrimidin-2-yl)-6-(2,2,2-trifluoroethyl)-5,6,7,8-tetrahydro-1,6-naphthyridin-3-amine as pink solid (73.70 mg; 35.8%).

HPLC 98.36% purity, 3.19 min. MS: m/z=513.25 [M+H]⁺. ¹H NMR (400 MHz, DMSO-d6, ppm): 9.62 (s, 1H), 8.64 (d, J=2.5 Hz, 1H), 8.35 (s, 1H), 7.91 (d, J=2.5 Hz, 1H), 7.29 (s, 1H), 5.53 (d, J=2.9 Hz, 1H), 4.23-4.16 (m, 2H), 3.96 (s, 2H), 3.83 (s, 2H), 3.44-3.36 (m, 2H), 3.30 (d, J=4.5 Hz, 2H), 3.16-3.09 (m, 2H), 3.05-2.97 (m, 2H), 2.81 (dt, J=11.8, 5.9 Hz, 4H), 2.04 (s, 3H).

### Example 107: Synthesis of Compound 107 (5-((7-(8-methyl-2,3-dihydro-1H-pyrido[2,3-b][1,4]oxazin-7-yl)-5,6,7,8-tetrahydropyrido[3,4-d]pyrimidin-2-yl)amino)-2-(1-methylpiperidin-4-yl)phenyl)methanol

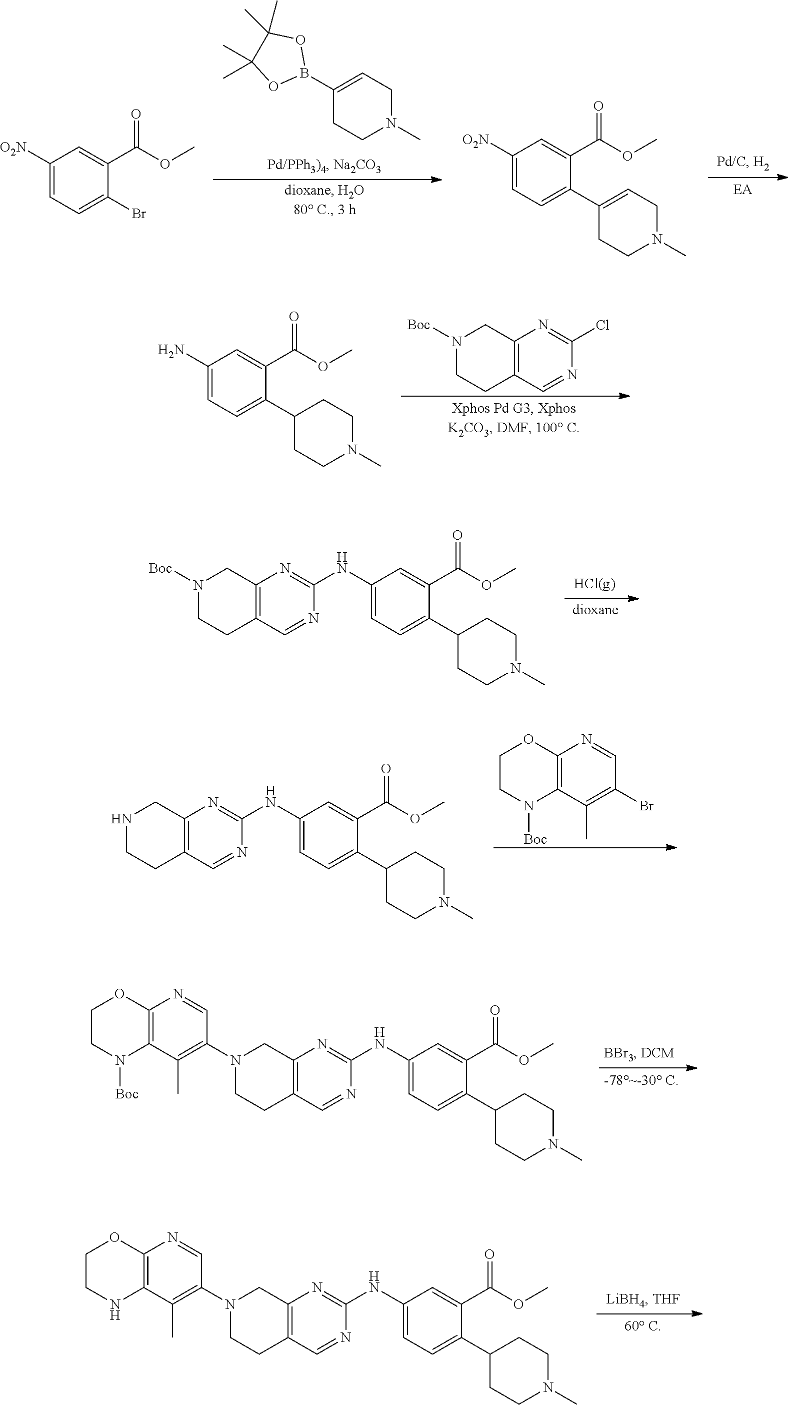

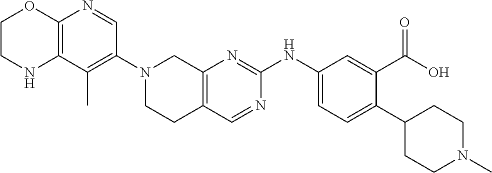 [SMILES: Cc1c(N2CCc3cnc(Nc4ccc(C5CCN(C)CC5)c(C(=O)O)c4)nc3C2)cnc2c1NCCO2]

methyl 2-(1-methyl-1,2,3,6-tetrahydropyridin-4-yl)-5-nitrobenzoate: To a stirred solution of methyl 2-bromo-5-nitrobenzoate (1 g; 3.65 mmol) and 1-methyl-4-(4,4,5,5-tetramethyl-1,3,2-dioxaborolan-2-yl)-1,2,3,6-tetrahydropyridine (1.0 g; 4.26 mmol) in 1,4-dioxane (20 mL) and H₂O (2 mL) was added Pd(PPh3)4 (1.0 g; 0.82 mmol) K₂CO₃ (1.0 g; 6.87 mmol). The resulting mixture was stirred for 2 h at 80 degrees C. under nitrogen atmosphere. The mixture was concentrated under reduced pressure. The residue was purified by Prep-HPLC (CH₂Cl₂/MeOH 4:1) to afford methyl 2-(1-methyl-1,2,3,6-tetrahydropyridin-4-yl)-5-nitrobenzoate as yellow oil (1.00 g; 99.1%).

methyl 5-amino-2-(1-methylpiperidin-4-yl)benzoate: To a stirred solution of methyl 2-(1-methyl-1,2,3,6-tetrahydropyridin-4-yl)-5-nitrobenzoate (500 mg; 1.81 mmol) in ethyl acetate (10 mL) was added Pd/C (100 mg; 3.62 mmol). The mixture was hydrogenated at room temperature for 2 h under hydrogen atmosphere using a hydrogen balloon. The solid was filtered out. The filtrate was concentrated under vacuum to afford methyl 5-amino-2-(1-methylpiperidin-4-yl)benzoate as yellow oil (490 mg; Crude Product).

tert-butyl 2-{[3-(methoxycarbonyl)-4-(1-methylpiperidin-4-yl)phenyl]amino}-5H,6H,7H,8H-pyrido[3,4-d]pyrimidine-7-carboxylate: To a stirred solution of methyl 5-amino-2-(1-methylpiperidin-4-yl)benzoate (480 mg; 1.74 mmol) and tert-butyl 2-chloro-5H,6H,7H,8H-pyrido[3,4-d]pyrimidine-7-carboxylate (560 mg; 2.08 mmol) in DMF (10 mL) was added XPhos Pd G3 (310 mg; 0.35 mmol), X-PHOS (175 mg; 0.35 mmol) and K₂CO₃ (510 mg; 3.51 mmol). The resulting mixture was stirred for 2 h at 90 degrees C. under nitrogen atmosphere. The mixture was concentrated under reduced pressure. The residue was purified by Prep-HPLC (CH₂Cl₂/MeOH 4:1) to afford tert-butyl 2-{[3-(methoxycarbonyl)-4-(1-methylpiperidin-4-yl)phenyl]amino}-5H,6H,7H,8H-pyrido[3,4-d]pyrimidine-7-carboxylate as yellow solid (600 mg; 63.9%).

methyl 2-(1-methylpiperidin-4-yl)-5-({5H,6H,7H,8H-pyrido[3,4-d]pyrimidin-2-yl}amino)benzoate: To a stirred solution of tert-butyl 2-{[3-(methoxycarbonyl)-4-(1-methylpiperidin-4-yl)phenyl]amino}-5H,6H,7H,8H-pyrido[3,4-d]pyrimidine-7-carboxylate (590 mg; 1.09 mmol) in 1,4-dioxane (5 mL) was added HCl (gas) in 1,4-dioxane (5 mL; 20.00 mmol). The resulting mixture was stirred for 2 h at room temperature. The resulting mixture was concentrated under vacuum to afford methyl 2-(1-methylpiperidin-4-yl)-5-({5H,6H,7H,8H-pyrido[3,4-d]pyrimidin-2-yl}amino)benzoate as yellow solid (380 mg; Crude Product).

tert-butyl 7-(2-{[3-(methoxycarbonyl)-4-(1-methylpiperidin-4-yl)phenyl]amino}-5H,6H,7H,8H-pyrido[3,4-d]pyrimidin-7-yl)-8-methyl-1H,2H,3H-pyrido[2,3-b][1,4]oxazine-1-carboxylate: To a stirred solution of methyl 2-(1-methylpiperidin-4-yl)-5-({5H,6H,7H,8H-pyrido[3,4-d]pyrimidin-2-yl}amino)benzoate (370 mg; 0.97 mmol) and tert-butyl 7-bromo-8-methyl-1H,2H,3H-pyrido[2,3-b][1,4]oxazine-1-carboxylate (480 mg; 1.46 mmol) in DMF (10 mL) was added Pd-PEPPSI-IPentCl 2-methylpyridine (o-picoline) (170 mg; 0.19 mmol) and Cs₂CO₃ (950 mg; 2.89 mmol). The resulting mixture was stirred for 14 h at 90 degrees C. under nitrogen atmosphere. The mixture was concentrated under reduced pressure. The residue was purified by Prep-HPLC (CH₂Cl₂/MeOH 4:1) to afford tert-butyl 7-(2-{[3-(methoxycarbonyl)-4-(1-methylpiperidin-4-yl)phenyl]amino}-5H,6H,7H,8H-pyrido[3,4-d]pyrimidin-7-yl)-8-methyl-1H,2H,3H-pyrido[2,3-b][1,4]oxazine-1-carboxylate as yellow solid (300 mg; 43.4%).

methyl 5-[(7-{8-methyl-1H,2H,3H-pyrido[2,3-b][1,4]oxazin-7-yl}-5H,6H,7H,8H-pyrido[3,4-d]pyrimidin-2-yl)amino]-2-(1-methylpiperidin-4-yl)benzoate: To a stirred solution of tert-butyl 7-(2-{[3-(methoxycarbonyl)-4-(1-methylpiperidin-4-yl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (280 mg; 0.39 mmol) in DCM (10 mL) was added BBr₃ in DCM (2 mL; 2.00 mmol) at −30 degrees C. under nitrogen atmosphere. The resulting mixture was stirred for 2 h at −30 degrees C. under nitrogen atmosphere. The residue was diluted with water, then adjusted to pH 6-7 with sodium bicarbonate, then extracted with EtOAc. The combined organic layer was dried over anhydrous sodium sulfate and concentrated under vacuum to afford methyl 5-[(7-{8-methyl-1H,2H,3H-pyrido[2,3-b][1,4]oxazin-7-yl}-5H,6H,7H,8H-pyrido[3,4-d]pyrimidin-2-yl)amino]-2-(1-methylpiperidin-4-yl)benzoate as yellow solid (165 mg; Crude Product).

{5-[(7-{8-methyl-1H,2H,3H-pyrido[2,3-b][1,4]oxazin-7-yl}-5H,6H,7H,8H-pyrido[3,4-d]pyrimidin-2-yl)amino]-2-(1-methylpiperidin-4-yl)phenyl}methanol: To a stirred solution of methyl 5-[(7-{8-methyl-1H,2H,3H-pyrido[2,3-b][1,4]oxazin-7-yl}-5H,6H,7H,8H-pyrido[3,4-d]pyrimidin-2-yl)amino]-2-(1-methylpiperidin-4-yl)benzoate (130 mg; 0.25 mmol) in THF (8 mL) was added diisobutylaluminum hydride, 1.0M in hexanes (4.0 mL; 4.00 mmol) at 0 degrees C. under nitrogen atmosphere. The resulting mixture was stirred for 2 h at 0 degrees C. under nitrogen atmosphere. The residue was purified by Prep-HPLC with the following conditions (Column: XBridge Shield RP18 OBD Column, 30*150 mm, 5 μm; Mobile Phase A: water (10 mmol/L NH₄HCO₃+0.05% NH₄OH), Mobile Phase B: ACN; Flow rate:60 mL/min.; Gradient: 20 B to 50 B in 9 min.; 254 nm; RT1:7) to afford {5-[(7-{8-methyl-1H,2H,3H-pyrido[2,3-b][1,4]oxazin-7-yl}-5H,6H,7H,8H-pyrido[3,4-d]pyrimidin-2-yl)amino]-2-(1-methylpiperidin-4-yl)phenyl}methanol as white solid (18.10 mg; 14.6%).

HPLC 99.45% purity, 3.43 min. MS: m/z=502.35 [M+H]⁺. ¹H NMR (400 MHz, DMSO-d6) δ 9.39 (s, 1H), 8.30 (s, 1H), 7.64 (dq, J=3.7, 2.4 Hz, 2H), 7.28 (s, 1H), 7.16-7.09 (m, 1H), 5.53 (t, J=2.7 Hz, 1H), 5.01 (t, J=5.4 Hz, 1H), 4.48 (d, J=4.9 Hz, 2H), 4.22-4.15 (m, 2H), 3.93 (s, 2H), 3.30 (p, J=3.5 Hz, 2H), 3.11 (t, J=5.7 Hz, 2H), 2.85 (dd, J=8.9, 5.5 Hz, 2H), 2.81-2.74 (m, 2H), 2.65 (tt, J=10.4, 5.3 Hz, 1H), 2.18 (s, 3H), 2.04 (s, 3H), 1.94 (td, J=11.0, 4.3 Hz, 2H), 1.72-1.58 (m, 4H).

### Example 108: Synthesis of Compound 108 7-(8-methyl-2,3-dihydro-1H-pyrido[2,3-b][1,4]oxazin-7-yl)-N-(5,6,7,8-tetrahydro-1,6-naphthyridin-3-yl)-5,6,7,8-tetrahydropyrido[3,4-d]pyrimidin-2-amine

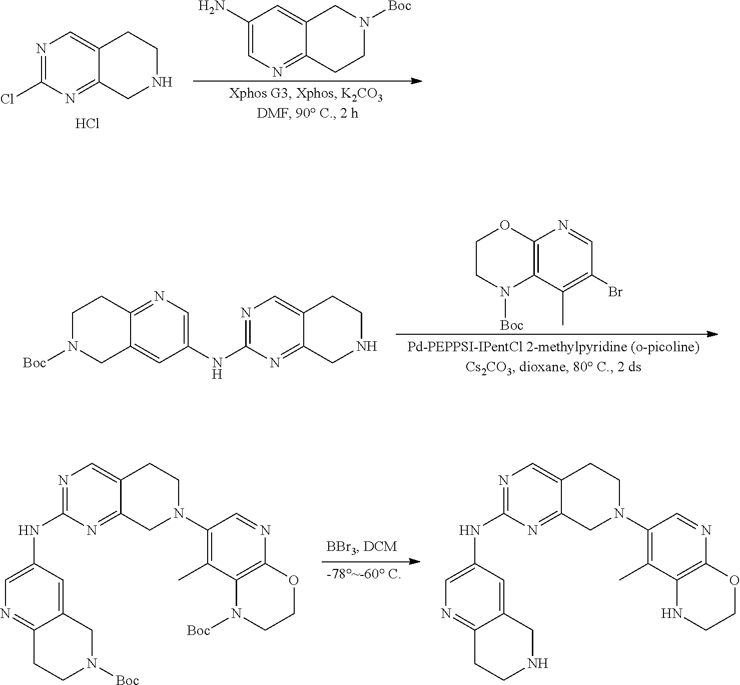

tert-butyl 3-({5H,6H,7H,8H-pyrido[3,4-d]pyrimidin-2-yl}amino)-5,6,7,8-tetrahydro-1,6-naphthyridine-6-carboxylate: To a stirred solution of 2-chloro-5H,6H,7H,8H-pyrido[3,4-d]pyrimidine hydrochloride (400 mg; 1.84 mmol) and tert-butyl 3-amino-5,6,7,8-tetrahydro-1,6-naphthyridine-6-carboxylate (530 mg; 2.02 mmol) in DMF (15 mL) was added X-PHOS (190 mg; 0.38 mmol) and XPhos Pd G3 (180 mg; 0.20 mmol). The resulting mixture was stirred for 3 h at 90 degrees C. under nitrogen atmosphere. The resulting mixture was concentrated under vacuum. The residue was purified by silica gel column chromatography and eluted with PE/EtOAc CH₂Cl₂/MeOH (1:1) to afford tert-butyl 3-({5H,6H,7H,8H-pyrido[3,4-d]pyrimidin-2-yl}amino)-5,6,7,8-tetrahydro-1,6-naphthyridine-6-carboxylate brown solid (450 mg 63.8%).

tert-butyl 3-[(7-{1-[(tert-butoxy)carbonyl]-8-methyl-1H,2H,3H-pyrido[2,3-b][1,4]oxazin-7-yl}-5H,6H,7H,8H-pyrido[3,4-d]pyrimidin-2-yl)amino]-5,6,7,8-tetrahydro-1,6-naphthyridine-6-carboxylate: To a stirred solution of tert-butyl 3-({5H,6H,7H,8H-pyrido[3,4-d]pyrimidin-2-yl}amino)-5,6,7,8-tetrahydro-1,6-naphthyridine-6-carboxylate (300 mg; 0.78 mmol) and tert-butyl 7-bromo-8-methyl-1H,2H,3H-pyrido[2,3-b][1,4]oxazine-1-carboxylate (500 mg; 1.52 mmol) in 1,4-dioxane (10 mL) was added Cs₂CO₃ (900 mg; 2.73 mmol) and Pd-PEPPSI-IPentCl 2-methylpyridine (o-picoline) (150 mg; 0.17 mmol) under nitrogen atmosphere. The resulting mixture was stirred for overnight at 80 degrees C. under nitrogen atmosphere. The resulting mixture was concentrated under vacuum. The residue was purified by silica gel column chromatography and eluted with CH₂Cl₂/MeOH (1:1). This resulted in tert-butyl 3-[(7-{1-[(tert-butoxy)carbonyl]-8-methyl-1H,2H,3H-pyrido[2,3-b][1,4]oxazin-7-yl}-5H,6H,7H,8H-pyrido[3,4-d]pyrimidin-2-yl)amino]-5,6,7,8-tetrahydro-1,6-naphthyridine-6-carboxylate as yellow solid (160 mg; 24.0%).

N-(7-{8-methyl-1H,2H,3H-pyrido[2,3-b][1,4]oxazin-7-yl}-5H,6H,7H,8H-pyrido[3,4-d]pyrimidin-2-yl)-5,6,7,8-tetrahydro-1,6-naphthyridin-3-amine: To a stirred solution of tert-butyl 3-[(7-{1-[(tert-butoxy)carbonyl]-8-methyl-1H,2H,3H-pyrido[2,3-b][1,4]oxazin-7-yl}-5H,6H,7H,8H-pyrido[3,4-d]pyrimidin-2-yl)amino]-5,6,7,8-tetrahydro-1,6-naphthyridine-6-carboxylate (160 mg; 0.01 mmol) in DCM (15 mL) was added BBr₃ in DCM (1 mL; 1.00 mmol) dropwise at −78 degrees C. under nitrogen atmosphere. The resulting mixture was stirred for 2 h at −78 degrees C. under nitrogen atmosphere. The resulting mixture was concentrated under vacuum. The residue was purified by silica gel column chromatography and eluted with CH₂Cl₂/MeOH (4:1) to afford crude product. The residue was purified by reverse flash chromatography with the following conditions: Column: XBridge Prep OBD C18 Column, 30×150 mm, 5 μm; Mobile Phase A: water (10 mmol/L NH₄HCO₃+0.05% NH₄OH), Mobile Phase B: ACN; Flow rate:60 mL/min.; Gradient:20 B to 50 B in 8 min.; 254 nm; RT1:6.6). This resulted in N-(7-{8-methyl-1H,2H,3H-pyrido[2,3-b][1,4]oxazin-7-yl}-5H,6H,7H,8H-pyrido[3,4-d]pyrimidin-2-yl)-5,6,7,8-tetrahydro-1,6-naphthyridin-3-amine as white solid (6.10 mg; 118.9%).

HPLC 98.57% purity, 2.08 min. MS: m/z=431.25 [M+H]⁺. ¹H NMR (300 MHz, DMSO-d6, ppm): 8.59 (t, J=1.9 Hz, 1H), 8.33 (s, 1H), 7.83 (d, J=2.5 Hz, 1H), 7.27 (s, 1H), 4.24-4.15 (m, 2H), 3.95 (s, 2H), 3.81 (s, 2H), 3.34-3.25 (m, 2H), 3.17-3.07 (m, 2H), 3.03-2.94 (m, 2H), 2.84-2.65 (m, 4H), 2.04 (s, 3H).

### Example 109: Synthesis of Compound 109 6-methyl-N-(6-(8-methyl-2,3-dihydro-1H-pyrido[2,3-b][1,4]oxazin-7-yl)-5,6,7,8-tetrahydro-2,6-naphthyridin-3-yl)-5,6,7,8-tetrahydro-1,6-naphthyridin-3-amine

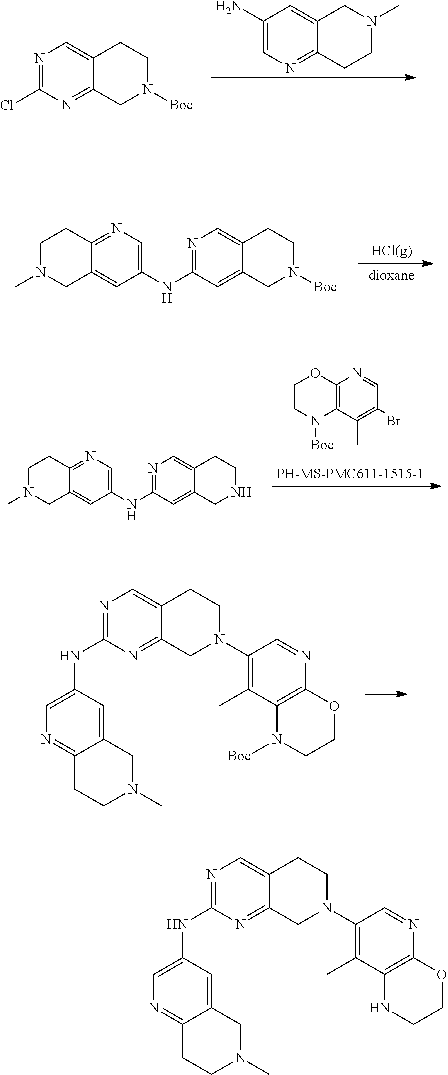

tert-butyl 7-[(6-methyl-5,6,7,8-tetrahydro-1,6-naphthyridin-3-yl)amino]-1,2,3,4-tetrahydro-2,6-naphthyridine-2-carboxylate: To a stirred solution of tert-butyl 7-chloro-1,2,3,4-tetrahydro-2,6-naphthyridine-2-carboxylate (500 mg; 1.77 mmol) and 6-methyl-5,6,7,8-tetrahydro-1,6-naphthyridin-3-amine (400 mg; 2.33 mmol) in 1,4-dioxane (10 mL) was added XPhos Pd G3 (750 mg; 0.84 mmol), 2-dicyclohexyl p-2 minus 2-3 isopropyl biphenyl (450 mg; 0.90 mmol) and sodium 2-methylpropan-2-olate (500 mg; 4.94 mmol). The resulting mixture was stirred for 4 h at 110 degrees C. under nitrogen atmosphere. The mixture was concentrated under reduced pressure. The residue was purified by Prep-HPLC (CH₂Cl₂/MeOH 4:1) to afford tert-butyl 7-[(6-methyl-5,6,7,8-tetrahydro-1,6-naphthyridin-3-yl)amino]-1,2,3,4-tetrahydro-2,6-naphthyridine-2-carboxylate as yellow solid (600 mg; 85.8%).

6-methyl-N-(5,6,7,8-tetrahydro-2,6-naphthyridin-3-yl)-5,6,7,8-tetrahydro-1,6-naphthyridin-3-amine: To a stirred solution of tert-butyl 7-[(6-methyl-5,6,7,8-tetrahydro-1,6-naphthyridin-3-yl)amino]-1,2,3,4-tetrahydro-2,6-naphthyridine-2-carboxylate (590 mg; 1.49 mmol) in 1,4-dioxane (10 mL) was added HCl (gas) in 1,4-dioxane (5 mL; 20.00 mmol). The resulting mixture was stirred for 2 h at 50 room temperature. The mixture was concentrated under reduced pressure to afford 6-methyl-N-(5,6,7,8-tetrahydro-2,6-naphthyridin-3-yl)-5,6,7,8-tetrahydro-1,6-naphthyridin-3-amine as yellow oil (400 mg; Crude Product).

tert-butyl 8-methyl-7-{7-[(6-methyl-5,6,7,8-tetrahydro-1,6-naphthyridin-3-yl)amino]-1,2,3,4-tetrahydro-2,6-naphthyridin-2-yl}-1H,2H,3H-pyrido[2,3-b][1,4]oxazine-1-carboxylate: To a stirred solution of 6-methyl-N-(5,6,7,8-tetrahydro-2,6-naphthyridin-3-yl)-5,6,7,8-tetrahydro-1,6-naphthyridin-3-amine (200 mg; 0.68 mmol) and tert-butyl 7-bromo-8-methyl-1H,2H,3H-pyrido[2,3-b][1,4]oxazine-1-carboxylate (450 mg; 1.37 mmol) in DMF (10 mL) was added Pd-PEPPSI-IPentCl 2-methylpyridine (o-picoline) (30 mg; 0.34 mmol) and Cs₂CO₃ (450 mg; 1.37 mmol). The resulting mixture was stirred for 12 h at 90 degrees C. under nitrogen atmosphere. The mixture was concentrated under reduced pressure. The residue was purified by Prep-HPLC (CH₂Cl₂/MeOH 4:1) to afford tert-butyl 8-methyl-7-{7-[(6-methyl-5,6,7,8-tetrahydro-1,6-naphthyridin-3-yl)amino]-1,2,3,4-tetrahydro-2,6-naphthyridin-2-yl}-1H,2H,3H-pyrido[2,3-b][1,4]oxazine-1-carboxylate as yellow solid (160 mg; 43.5%).

6-{8-methyl-1H,2H,3H-pyrido[2,3-b][1,4]oxazin-7-yl}-N-(6-methyl-5,6,7,8-tetrahydro-1,6-naphthyridin-3-yl)-5,6,7,8-tetrahydro-2,6-naphthyridin-3-amine: To a stirred solution of tert-butyl 8-methyl-7-{7-[(6-methyl-5,6,7,8-tetrahydro-1,6-naphthyridin-3-yl)amino]-1,2,3,4-tetrahydro-2,6-naphthyridin-2-yl}-1H,2H,3H-pyrido[2,3-b][1,4]oxazine-1-carboxylate (140 mg; 0.26 mmol) in DCM (5 mL) was added tribromoborane (0.50 mL; 5.02 mmol) at −70 degrees C. The resulting mixture was stirred for 2 h at −70 degrees C. under nitrogen atmosphere. The residue was purified by Prep-HPLC with the following conditions Column: XBridge Prep OBD C18 Column, 30×150 mm, 5 μm; Mobile Phase A: water (10 mmol/mL NH₄HCO₃+0.05% NH₄OH), Mobile Phase B:ACN; Flow rate:60 mL/min.; Gradient:15 B to 45 B in 10 min., RT1:8.7) to afford 6-{8-methyl-1H,2H,3H-pyrido[2,3-b][1,4]oxazin-7-yl}-N-(6-methyl-5,6,7,8-tetrahydro-1,6-naphthyridin-3-yl)-5,6,7,8-tetrahydro-2,6-naphthyridin-3-amine as white solid (5.40 mg; 4.7%).

HPLC99. 54% purity, 2.39 min. MS: m/z=444.25 [M+H]⁺. ¹H NMR (300 MHz, DMSO-d6) δ 8.46 (d, J=2.5 Hz, 1H), 7.99 (s, 1H), 7.81 (d, J=2.6 Hz, 1H), 7.23 (s, 1H), 6.58 (s, 1H), 4.18 (d, J=4.6 Hz, 2H), 3.97 (s, 2H), 3.47 (s, 2H), 3.27 (d, J=5.5 Hz, 2H), 3.06 (d, J=6.1 Hz, 2H), 2.80 (s, 4H), 2.67 (d, J=5.8 Hz, 2H), 2.33 (s, 3H), 2.01 (s, 3H). 44.25

### Example 110: Synthesis of Compound 110 3-((7-(8-methyl-2,3-dihydro-1H-pyrido[2,3-b][1,4]oxazin-7-yl)-5,6,7,8-tetrahydropyrido[3,4-d]pyrimidin-2-yl)amino)-5,6,7,8-tetrahydroquinolin-6-ol

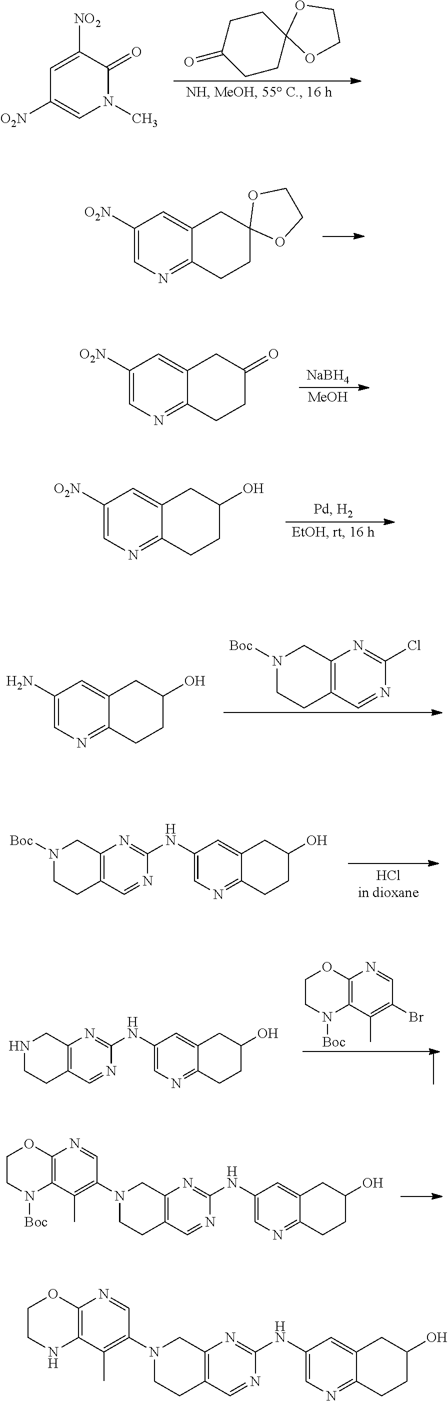

3′-nitro-7′,8′-dihydro-5′H-spiro[1,3-dioxolane-2,6′-quinoline]: To a stirred solution of 1-methyl-3,5-dinitro-1,2-dihydropyridin-2-one (2 g; 9.54 mmol) in NH₃ (g) in MeOH (20 mL; 140.00 mmol) was added 1,4-dioxaspiro[4.5]decan-8-one (2 g; 12.17 mmol). The resulting mixture was stirred for 16 h at 55 degrees C. under nitrogen atmosphere. The resulting mixture was concentrated under vacuum. The residue was purified by silica gel column chromatography and eluted with PE/EtOAc (3:1) to afford 3′-nitro-7′,8′-dihydro-5′H-spiro[1,3-dioxolane-2,6′-quinoline] as brown solid (1.70 g; 66.6%).

3-nitro-5,6,7,8-tetrahydroquinolin-6-one: To a stirred solution of 3′-nitro-7′,8′-dihydro-5′H-spiro[1,3-dioxolane-2,6′-quinoline] (1 g; 3.74 mmol) in DCM (50 mL) was added TFA (10 mL) at room temperature under nitrogen atmosphere. The resulting mixture was stirred for 5 days at 40 degrees C. under nitrogen atmosphere. After cooling, the mixture was poured in to saturated NaHCO₃ solution (100 mL) and extracted with CH₂Cl₂ (3×50 mL). The combined organic layers were washed with brine (100 mL), dried (Na₂SO₄), filtered, and concentrated under vacuum. Purification by column chromatography on silica gel using 0 to 5% MeOH in CH₂Cl₂ as eluent gave the compound 3-nitro-5,6,7,8-tetrahydroquinolin-6-one as yellow solid (450 mg; 62.7%).

3-nitro-5,6,7,8-tetrahydroquinolin-6-ol: To a stirred solution of 3-nitro-5,6,7,8-tetrahydroquinolin-6-one (450 mg; 2.34 mmol) in MeOH (10 mL) was added NaBH₄ (150 mg; 3.77 mmol) at room temperature under nitrogen atmosphere. The resulting mixture was stirred for 2 h at 25 degrees C. under nitrogen atmosphere. The resulting mixture was concentrated under reduced pressure. The residue was purified by silica gel column chromatography and eluted with PE/EtOAc (1:1) to afford 3-nitro-5,6,7,8-tetrahydroquinolin-6-ol as yellow solid (420 mg; 92.4%).

3-amino-5,6,7,8-tetrahydroquinolin-6-ol: To a solution of 3-nitro-5,6,7,8-tetrahydroquinolin-6-ol (420 mg) in EtOH (10 mL) was added Pd/C (50 mg; 0.14 mmol) under nitrogen atmosphere in a 50 mL round-bottom flask. The mixture was hydrogenated at room temperature for 1 h under hydrogen atmosphere using a hydrogen balloon. filtered through a Celite pad and concentrated under reduced pressure. This resulted in 3-amino-5,6,7,8-tetrahydroquinolin-6-ol as brown dark oil (380 mg; Crude Product).

tert-butyl 2-[(6-hydroxy-5,6,7,8-tetrahydroquinolin-3-yl)amino]-5H,6H,7H,8H-pyrido[3,4-d]pyrimidine-7-carboxylate: To a stirred solution of 3-amino-5,6,7,8-tetrahydroquinolin-6-ol (380 mg; 2.09 mmol) and tert-butyl 2-chloro-5H,6H,7H,8H-pyrido[3,4-d]pyrimidine-7-carboxylate (700 mg; 2.60 mmol) in DMF (10 mL) was added K₂CO₃ (750 mg; 5.16 mmol) X-PHOS (220 mg; 0.44 mmol) and XPhos Pd G3 (200 mg; 0.22 mmol). The resulting mixture was stirred for 4 h at 100 degrees C. under nitrogen atmosphere. The resulting mixture was extracted with EtOAc (3×30 mL). The combined organic layers were washed with brine and dried over anhydrous Na₂SO₄. After filtration, the filtrate was concentrated under reduced pressure. The residue was purified by silica gel column chromatography and eluted with PE/EtOAc CH₂Cl₂/MeOH (3:1) to afford tert-butyl 2-[(6-hydroxy-5,6,7,8-tetrahydroquinolin-3-yl)amino]-5H,6H,7H,8H-pyrido[3,4-d]pyrimidine-7-carboxylate as brown dark oil (500 mg; 60.1%).

3-({5H,6H,7H,8H-pyrido[3,4-d]pyrimidin-2-yl}amino)-5,6,7,8-tetrahydroquinolin-6-ol: To a stirred solution of tert-butyl 2-[(6-hydroxy-5,6,7,8-tetrahydroquinolin-3-yl)amino]-5H,6H,7H,8H-pyrido[3,4-d]pyrimidine-7-carboxylate (500 mg; 1.26 mmol) in 1,4-dioxane (10 mL) was added HCl (gas) in 1,4-dioxane (2 mL) dropwise at 0 degrees C. under nitrogen atmosphere. The resulting mixture was stirred for 2 h at room temperature under nitrogen atmosphere. The resulting mixture was concentrated under vacuum. This resulted in 3-({5H,6H,7H,8H-pyrido[3,4-d]pyrimidin-2-yl}amino)-5,6,7,8-tetrahydroquinolin-6-ol as brown solid (390 mg; Crude Product).

tert-butyl 7-{2-[(6-hydroxy-5,6,7,8-tetrahydroquinolin-3-yl)amino]-5H,6H,7H,8H-pyrido[3,4-d]pyrimidin-7-yl}-8-methyl-1H,2H,3H-pyrido[2,3-b][1,4]oxazine-1-carboxylate: To a stirred solution of 3-({5H,6H,7H,8H-pyrido[3,4-d]pyrimidin-2-yl}amino)-5,6,7,8-tetrahydroquinolin-6-ol (200 mg; 0.64 mmol) and tert-butyl 7-bromo-8-methyl-1H,2H,3H-pyrido[2,3-b][1,4]oxazine-1-carboxylate (350 mg; 1.01 mmol) in DMF (15.00 mL) was Cs₂CO₃ (600 mg; 1.82 mmol) and Pd-PEPPSI-IPentCl 2-methylpyridine (o-picoline) (120 mg; 0.14 mmol) under nitrogen atmosphere. The resulting mixture was stirred for 3 days at 90 degrees C. under nitrogen atmosphere. The resulting mixture was concentrated under vacuum. The residue was purified by silica gel column chromatography and eluted with CH₂Cl₂/MeOH (1:2) to afford. This resulted in tert-butyl 7-{2-[(6-hydroxy-5,6,7,8-tetrahydroquinolin-3-yl)amino]-5H,6H,7H,8H-pyrido[3,4-d]pyrimidin-7-yl}-8-methyl-1H,2H,3H-pyrido[2,3-b][1,4]oxazine-1-carboxylate as yellow solid (100 mg; 28.8%).

3-[(7-{8-methyl-1H,2H,3H-pyrido[2,3-b][1,4]oxazin-7-yl}-5H,6H,7H,8H-pyrido[3,4-d]pyrimidin-2-yl)amino]-5,6,7,8-tetrahydroquinolin-6-01: To a stirred solution of tert-butyl 7-{2-[(6-hydroxy-5,6,7,8-tetrahydroquinolin-3-yl)amino]-5H,6H,7H,8H-pyrido[3,4-d]pyrimidin-7-yl}-8-methyl-1H,2H,3H-pyrido[2,3-b][1,4]oxazine-1-carboxylate (100 mg; 0.18 mmol) in DCM (20 mL) was added BBr3 in DCM (1.00 mL; 1.00 mmol) at −78 degrees C. The resulting mixture was stirred for 2 h at −78 degrees C. under nitrogen atmosphere. The resulting mixture was concentrated under vacuum. The residue was purified by silica gel column chromatography and eluted with CH₂Cl₂/MeOH (1:1) to afford product. The product was purified by reverse flash chromatography with the following conditions: Column: XBridge Prep OBD C18 Column, 30×150 mm, 5 μm; Mobile Phase A: water (10 mmol/mL NH₄HCO₃+0.05% NH₄OH), Mobile Phase B: ACN; Flow rate:60 mL/min.; Gradient:20 B to 50 B in 8 min., 254 nm; RT1:6.54. This resulted in 3-[(7-{8-methyl-1H,2H,3H-pyrido[2,3-b][1,4]oxazin-7-yl}-5H,6H,7H,8H-pyrido[3,4-d]pyrimidin-2-yl)amino]-5,6,7,8-tetrahydroquinolin-6-ol as off-white solid (14.4 mg; 17.5%).

HPLC 99. 46% purity, 3.04 min. MS: m/z=446.15 [M+H]⁺. ¹H NMR (400 MHz, DMSO-d6, ppm): 9.51 (s, 1H), 8.60 (d, J=2.5 Hz, 1H), 8.34 (s, 1H), 7.82 (d, J=2.5 Hz, 1H), 7.28 (s, 1H), 5.53 (d, J=2.6 Hz, 1H), 4.81 (d, J=3.6 Hz, 1H), 4.22-4.15 (m, 2H), 3.95 (s, 3H), 3.31-3.26 (m, 2H), 3.15-3.08 (m, 2H), 2.90-2.59 (m, 6H), 2.03 (s, 3H), 1.92 (d, J=12.0 Hz, 1H), 1.82-1.71 (m, 2H).

### Example 111: Synthesis of Compound 111 1-(4-((7-(8-methyl-2,3-dihydro-1H-pyrido[2,3-b][1,4]oxazin-7-yl)-5,6,7,8-tetrahydropyrido[3,4-d]pyrimidin-2-yl)amino)phenyl)piperidin-4-ol

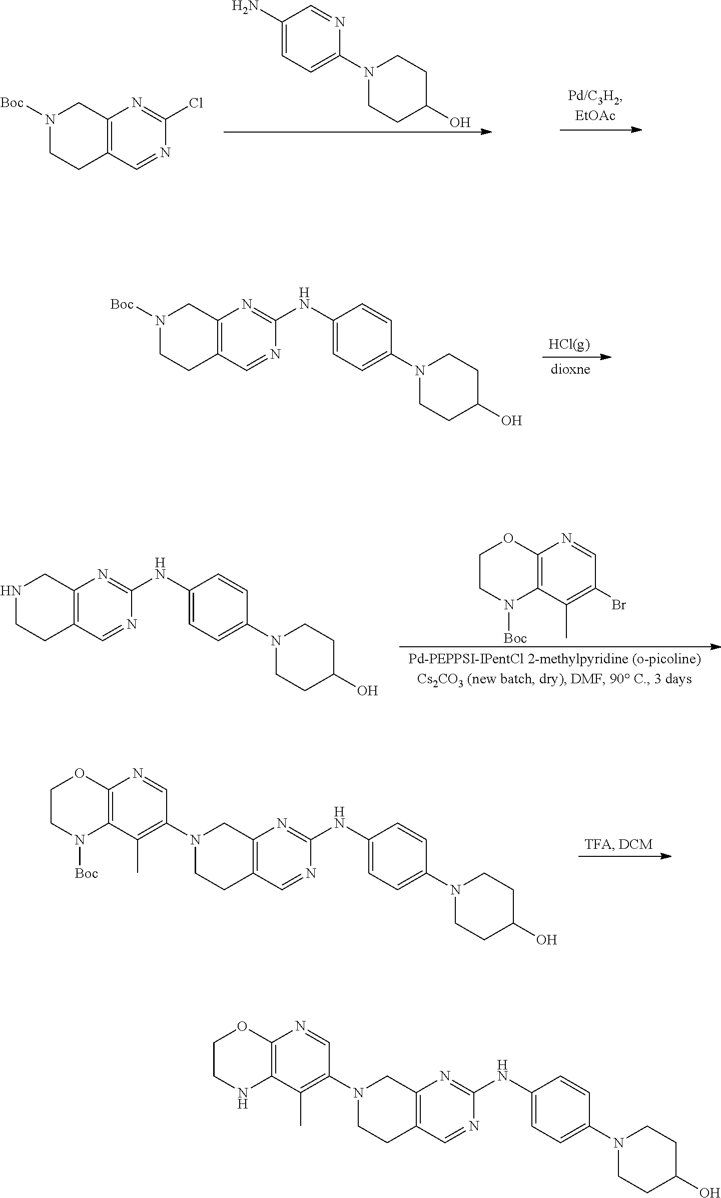

2-{[4-(4-methoxypiperidin-1-yl)phenyl]amino}-5H,6H,7H,8H-pyrido[3,4-d]pyrimidine-7-carboxylate: To a stirred solution of 4-(4-methoxypiperidin-1-yl)aniline (400 mg; 1.94 mmol) and in DMF (10 mL) was added K₂CO₃ (540 mg; 3.71 mmol) X-PHOS (190 mg; 0.38 mmol) and XPhos Pd G3 (170 mg; 0.19 mmol). The resulting mixture was stirred for 2 h at 100 degrees C. under nitrogen atmosphere. The resulting mixture was concentrated under vacuum. The residue was purified by silica gel column chromatography and eluted with PE/EtOAc CH₂Cl₂/MeOH (1:1) to afford tert-butyl 2-{[4-(4-methoxypiperidin-1-yl)phenyl]amino}-5H,6H,7H,8H-pyrido[3,4-d]pyrimidine-7-carboxylate as brown solid (800 mg; 88.1%).

N-[4-(4-methoxypiperidin-1-yl)phenyl]-5H,6H,7H,8H-pyrido[3,4-d]pyrimidin-2-amine: To a stirred solution of tert-butyl 2-{[4-(4-methoxypiperidin-1-yl)phenyl]amino}-5H,6H,7H,8H-pyrido[3,4-d]pyrimidine-7-carboxylate (750 mg; 1.60 mmol) in 1,4-dioxane (10 mL) was added HCl (gas) in 1,4-dioxane (2 mL). The resulting mixture was stirred for 2 h at room temperature under nitrogen atmosphere. The resulting mixture was concentrated under vacuum. This resulted in N-[4-(4-methoxypiperidin-1-yl)phenyl]-5H,6H,7H,8H-pyrido[3,4-d]pyrimidin-2-amine as brown solid (500 mg; Crude).

3-[(7-[8-methyl-1H,2H,3H-pyrido[2,3-b][1,4]oxazin-7-yl]-5H,6H,8H-pyrido[3,4-d]pyrimidin-2-yl)amino]-5,6,7,8-tetrahydroquinolin-6-ol: To a stirred solution of N-[4-(4-methoxypiperidin-1-yl)phenyl]-5H,6H,7H,8H-pyrido[3,4-d]pyrimidin-2-amine (200 mg; 0.59 mmol) and tert-butyl 7-bromo-8-methyl-2H,3H-pyrido[2,3-b][1,4]oxazine-1-carboxylate (340 mg; 1.00 mmol) in 1,4-dioxane (10 mL) was added Cs₂CO₃ (500 mg; 1.52 mmol) and Pd-PEPPSI-IPentCl 2-methylpyridine (o-picoline) (60 mg; 0.07 mmol). The resulting mixture was stirred for 3 days at 90 degrees C. under nitrogen atmosphere. The resulting mixture was concentrated under vacuum. The residue was purified by silica gel column chromatography and eluted with CH₂Cl₂/MeOH (1:1) to afford 3-[(7-[8-methyl-1H,2H,3H-pyrido[2,3-b][1,4]oxazin-7-yl]-5H,6H,8H-pyrido[3,4-d]pyrimidin-2-yl)amino]-5,6,7,8-tetrahydroquinolin-6-ol as yellow solid (200 mg; 57.8%).

1-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}piperidin-4-ol: To a stirred solution of 3-[(7-[8-methyl-1H,2H,3H-pyrido[2,3-b][1,4]oxazin-7-yl]-5H,6H,8H-pyrido[3,4-d]pyrimidin-2-yl)amino]-5,6,7,8-tetrahydroquinolin-6-ol (160 mg; 0.27 mmol) in DCM (10 mL) was added tribromoborane (1.13 mL; 11.38 mmol) at −70 degrees C. The resulting mixture was stirred for 6 h at −70 degrees C. under nitrogen atmosphere. The reaction was quenched by MeOH at −78 degree C. and concentrated under vacuum. The residue was purified by Prep-HPLC with the following conditions (Column: XBridge Prep OBD C18 Column, 30×150 mm, 5 μm; Mobile Phase A: water (10 mmol/L NH₄HCO₃+0.05% NH₄OH), Mobile Phase B:ACN; Flow rate:60 mL/min.; Gradient:30 B to 60 B in 8 min., 254 nm; RT1:6.5) to afford 1-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}piperidin-4-ol as white solid (10.30 mg; 7.8%).

HPLC 97.82% purity, 3.04 min., MS: m/s=474.20 [M+H]⁺. ¹H NMR (300 MHz, DMSO-d6) 9.20 (s, 1H), 8.27 (s, 1H), 7.55 (d, J=8.9 Hz, 2H), 7.28 (s, 1H), 6.87 (d, J=9.0 Hz, 2H), 5.52 (s, 1H), 4.65 (d, J=4.2 Hz, 1H), 4.20 (s, 2H), 3.92 (s, 2H), 2.77 (s, 4H), 2.05 (s, 3H), 1.82 (d, J=12.1 Hz, 2H), 1.49 (q, J=9.4 Hz, 4H).

### Example 112: Synthesis of Compound 112 2-(5-((7-(8-methyl-2,3-dihydro-1H-pyrido[2,3-b][1,4]oxazin-7-yl)-5,6,7,8-tetrahydropyrido[3,4-d]pyrimidin-2-yl)amino)-2-(1-methylpiperidin-4-yl)phenyl)acetonitrile

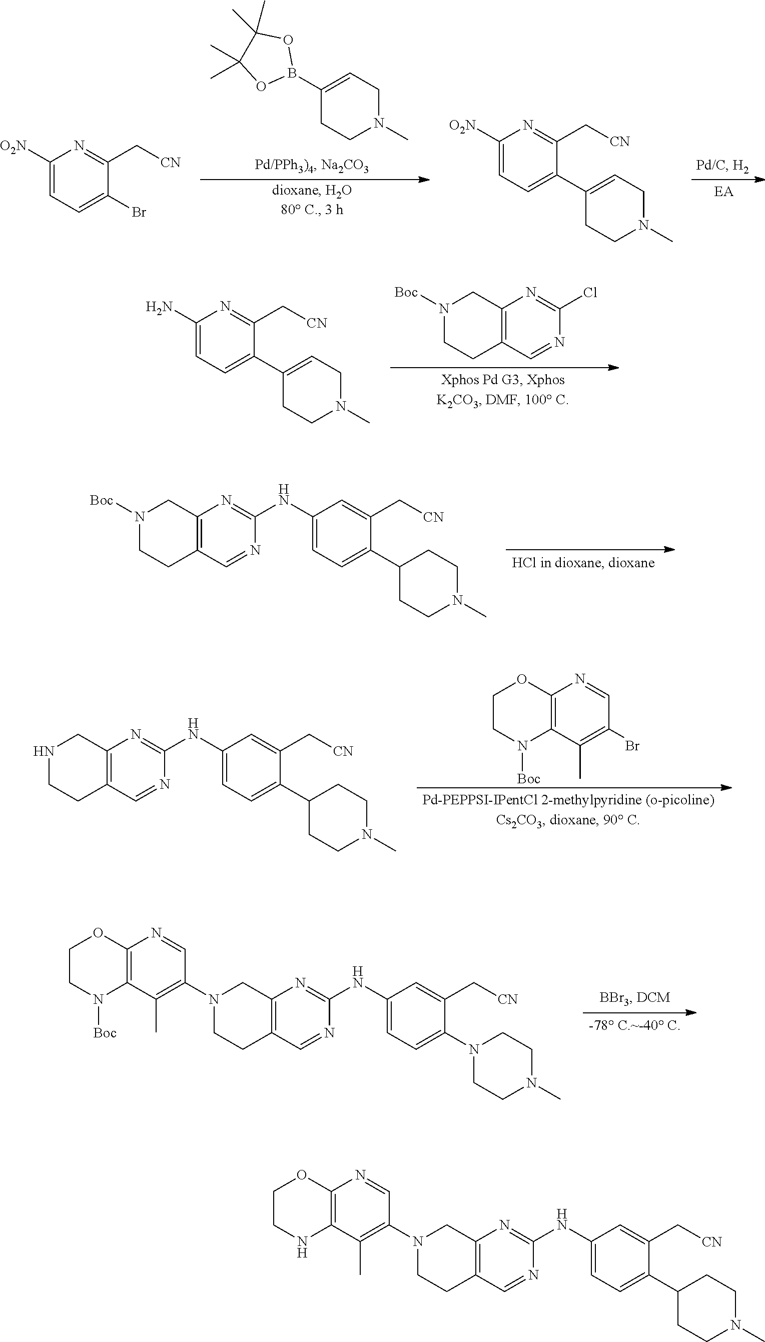

2-[2-(1-methyl-1,2,3,6-tetrahydropyridin-4-yl)-5-nitrophenyl]acetonitrile: To a stirred solution of 2-(2-bromo-5-nitrophenyl)acetonitrile (1 g; 3.94 mmol) 1-methyl-4-(4,4,5,5-tetramethyl-1,3,2-dioxaborolan-2-yl)-1,2,3,6-tetrahydropyridine (1.40 g; 5.96 mmol) and K₂CO₃ (1.20 g; 8.25 mmol) in 1,4-dioxane (10 mL) and water (1 mL) was added Pd(PPh3)₄ (480 mg; 0.39 mmol) at 25 degrees C. The resulting mixture was stirred for 4 h at 80 degrees under N₂ atmosphere. The resulting mixture was concentrated under reduced pressure. The residue was purified by silica gel column chromatography and eluted with MeOH:DCM (1:10) to afford 2-[2-(1-methyl-1,2,3,6-tetrahydropyridin-4-yl)-5-nitrophenyl]acetonitrile as yellow solid (800 mg; 78.9%).

2-[5-amino-2-(1-methylpiperidin-4-yl)phenyl]acetonitrile: To a solution of 2-[2-(1-methyl-1,2,3,6-tetrahydropyridin-4-yl)-5-nitrophenyl]acetonitrile (800 mg; 3.11 mmol) in AcOEt (10 mL) and MeOH (1 mL) was added Pd/C (100 mg; 0.09 mmol) under nitrogen atmosphere in a 50 mL round-bottom flask. The mixture was hydrogenated at room temperature for 16 h under hydrogen atmosphere using a hydrogen balloon. filtered through a Celite pad and concentrated under reduced pressure. This resulted in 2-[5-amino-2-(1-methylpiperidin-4-yl)phenyl]acetonitrile as yellow brown solid (400 mg, Crude Product).

tert-butyl 2-{[3-(cyanomethyl)-4-(1-methylpiperidin-4-yl)phenyl]amino}-5H,6H,7H,8H-pyrido[3,4-d]pyrimidine-7-carboxylate: To a stirred solution of 2-[5-amino-2-(1-methylpiperidin-4-yl)phenyl]acetonitrile (400 mg; 1.31 mmol) and tert-butyl 2-chloro-5H,6H,7H,8H-pyrido[3,4-d]pyrimidine-7-carboxylate (700 mg; 2.60 mmol) in DMF (15 mL) was added K₂CO₃ (400 mg; 2.75 mmol) X-PHOS (150 mg; 0.30 mmol) and XPhos Pd G3 (130 mg; 0.15 mmol). The resulting mixture was stirred for 4 h at 90 degrees C. under nitrogen atmosphere. The solvent was removed under vacuum. The residue was purified by silica gel column chromatography and eluted with CH₂Cl₂/MeOH (5:1) to afford. This resulted in tert-butyl 2-{[3-(cyanomethyl)-4-(1-methylpiperidin-4-yl)phenyl]amino}5H,6H,7H,8H-pyrido[3,4-d]pyrimidine-7-carboxylate as brown solid (250 mg, 41.1%).

2-[2-(1-methylpiperidin-4-yl)-5-({5H,6H,7H,8H-pyrido[3,4-d]pyrimidin-2-yl}amino)phenyl]acetonitrile: To a stirred solution of tert-butyl 2-{[3-(cyanomethyl)-4-(1-methylpiperidin-4-yl)phenyl]amino}-5H,6H,7H,8H-pyrido[3,4-d]pyrimidine-7-carboxylate (250 mg; 0.54 mmol) in 1,4-dioxane (5 mL) was added HCl (gas) in 1,4-dioxane (2 mL; 20 mmol) dropwise at 0 degrees C. The resulting mixture was stirred for 2 h at room temperature. The resulting mixture was concentrated under reduced pressure. This resulted in 2-[2-(1-methylpiperidin-4-yl)-5-({5H,6H,7H,8H-pyrido[3,4-d]pyrimidin-2-yl}amino)phenyl]acetonitrile as brown solid (170 mg; Crude Product).

tert-butyl7-(2-{[3-(cyanomethyl)-4-(1-methylpiperidin-4-yl)phenyl]amino}-5H,6H,7H,8H-pyrido[3,4-d]pyrimidin-7-yl)-8-methyl-1H,2H,3H-pyrido[2,3-b][1,4]oxazine-1-carboxylate: To a stirred solution of 2-[2-(1-methylpiperidin-4-yl)-5-({5H,6H,7H,8H-pyrido[3,4-d]pyrimidin-2-yl}amino)phenyl]acetonitrile (150 mg; 0.41 mmol) and tert-butyl 7-bromo-8-methyl-2,3-dihydro-TH-pyrido[2,3-b][1,4]oxazine-1-carboxylate (149.5 mg, 0.455 mmol) in 1,4-dioxane (15 mL) was added Cs₂CO₃ (400 mg; 1.22 mmol) and Pd-PEPPSI-IPentCl 2-methylpyridine (o-picoline) (50 mg; 0.06 mmol) under nitrogen atmosphere. The resulting mixture was stirred for overnight at 90 degrees C. under nitrogen atmosphere. The resulting mixture was concentrated under vacuum. The residue was purified by silica gel column chromatography and eluted with CH₂Cl₂/MeOH (1:2) to afford tert-butyl 7-(2-{[3-(cyanomethyl)-4-(1-methylpiperidin-4-yl)phenyl]amino}-5H,6H,7H,8H-pyrido[3,4-d]pyrimidin-7-yl)-8-methyl-1H,2H,3H-pyrido[2,3-b][1,4]oxazine-1-carboxylate as yellow solid (170 mg; 67.3%).

2-{5-[(7-{8-methyl-1H,2H,3H-pyrido[2,3-b][1,4]oxazin-7-yl}-5H,6H,7H,8H-pyrido[3,4-d]pyrimidin-2-yl)amino]-2-(1-methylpiperidin-4yl)phenyl}acetonitrile: To a stirred solution of tert-butyl 7-(2-{[3-(cyanomethyl)-4-(1-methylpiperidin-4-yl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (150 mg; 0.25 mmol) in DCM (15 mL) was added BBr₃ in DCM (0.50 mL; 0.50 mmol) dropwise at −78 degrees C. under nitrogen atmosphere. The resulting mixture was stirred for 3 h at −60 degrees C. under nitrogen atmosphere. The resulting mixture was concentrated under vacuum. The residue was purified by silica gel column chromatography and eluted with CH₂Cl₂/MeOH (1:1) to afford the crude product. The crude product was purified by reverse flash chromatography with the following conditions: Column: XBridge Prep OBD C18 Column, 30×150 mm, 5 μm; Mobile Phase A: water (10 mmol/mL NH₄HCO₃+0.05% NH₄OH), Mobile Phase B: ACN; Flow rate:60 mL/min.; Gradient: 20 B to 50 B in 8 min., 254 nm; RT1:6.5. This resulted in 2-{5-[(7-{8-methyl-1H,2H,3H-pyrido[2,3-b][1,4]oxazin-7-yl}-5H,6H,7H,8H-pyrido[3,4-d]pyrimidin-2-yl)amino]-2-(1-methylpiperidin-4-yl)phenyl}acetonitrile as white solid (13.20 mg; 10.4%).

HPLC 98.60% purity, 3.36 min, MS: m/s=511.25 [M+H]⁺. ¹H NMR (300 MHz, DMSO-d6, ppm): 9.53 (s, 1H), 8.31 (s, 1H), 7.88 (d, J=2.3 Hz, 1H), 7.56 (dd, J=8.6, 2.4 Hz, 1H), 7.26 (s, 1H), 7.18 (d, J=8.6 Hz, 1H), 5.52 (s, 1H), 4.22-4.13 (m, 2H), 3.97 (d, J=17.7 Hz, 4H), 3.29 (s, 2H), 3.10 (s, 2H), 2.84 (d, J=11.1 Hz, 3H), 2.77 (s, 2H), 2.54 (s, 1H), 2.18 (s, 3H), 2.02 (s, 5H), 1.62 (s, 4H).

### Example 113: Synthesis of Compound 113 N-(4-(4-methoxypiperidin-1-yl)phenyl)-7-(8-methyl-2,3-dihydro-1H-pyrido[2,3-b][1,4]oxazin-7-yl)-5,6,7,8-tetrahydropyrido[3,4-d]pyrimidin-2-amine

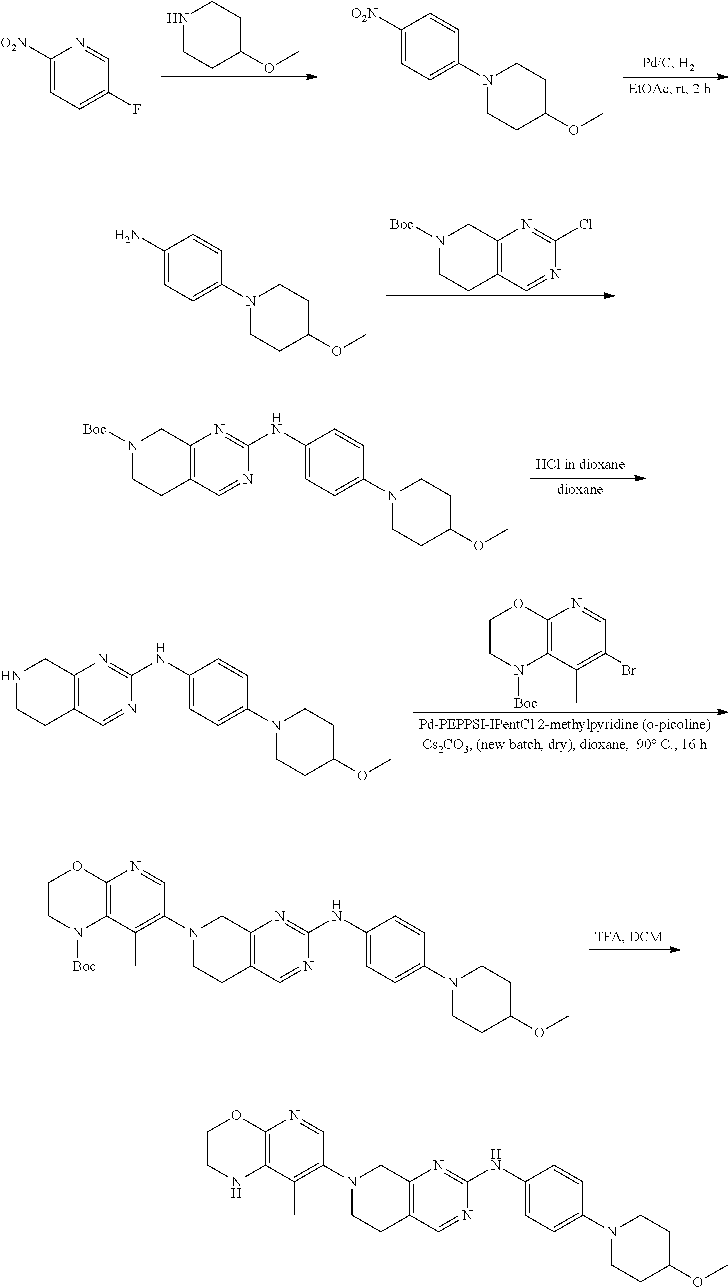

4-methoxy-1-(4-nitrophenyl)piperidine: To a stirred solution of 1-fluoro-4-nitrobenzene (1 g; 6.73 mmol) and 4-methoxypiperidine (1.60 g; 13.20 mmol) in DMF (20 mL) was added K₂CO₃ (2 g; 13.75 mmol). The resulting mixture was stirred for 2 h at room temperature. The resulting mixture was concentrated under vacuum. The residue was purified by silica gel column chromatography and eluted with PE/EtOAc (1:1) to afford 4-methoxy-1-(4-nitrophenyl)piperidine as yellow solid (1.40 g; 88.0%).

4-(4-methoxypiperidin-1-yl)aniline: To a solution of 4-methoxy-1-(4-nitrophenyl)piperidine (700 mg; 2.96 mmol) in 15 mL MeOH was added Pd/C (10%, 100 mg) under nitrogen atmosphere in a 50 mL round-bottom flask. The mixture was hydrogenated at room temperature for 1 h under hydrogen atmosphere using a hydrogen balloon. filtered through a Celite pad and concentrated under reduced pressure. This resulted in 4-(4-methoxypiperidin-1-yl)aniline as brown solid (600 mg; Crude Product).

tert-butyl2-{[4-(4-methoxypiperidin-1-yl)phenyl]amino}-5H,6H,7H,8H-pyrido[3,4-d]pyrimidine-7-carboxylate: To a stirred solution of 4-(4-methoxypiperidin-1-yl)aniline (600 mg; 1.94 mmol) and tert-butyl 2-chloro-5,8-dihydropyrido[3,4-d]pyrimidine-7(6H)-carboxylate (630 mg, 2.34 mmol) in DMF (10 mL) was added K₂CO₃ (540 mg; 3.71 mmol) X-PHOS (190 mg; 0.38 mmol) and XPhos Pd G3 (170 mg; 0.19 mmol). The resulting mixture was stirred for 2 h at 100 degrees C. under nitrogen atmosphere. The resulting mixture was concentrated under vacuum. The residue was purified by silica gel column chromatography and eluted with PE/EtOAc CH₂Cl₂/MeOH (1:1) to afford tert-butyl 2-{[4-(4-methoxypiperidin-1-yl)phenyl]amino}-5H,6H,7H,8H-pyrido[3,4-d]pyrimidine-7-carboxylate as brown solid (750 mg; 88.1%).

N-[4-(4-methoxypiperidin-1-yl)phenyl]-5H,6H,7H,8H-pyrido[3,4-d]pyrimidin-2-amine: To a stirred solution of tert-butyl 2-{[4-(4-methoxypiperidin-1-yl)phenyl]amino}-5H,6H,7H,8H-pyrido[3,4-d]pyrimidine-7-carboxylate (750 mg; 1.60 mmol) in 1,4-dioxane (10 mL) was added HCl (gas) in 1,4-dioxane (2 mL). The resulting mixture was stirred for 2 h at room temperature under nitrogen atmosphere. The resulting mixture was concentrated under vacuum. This resulted in N-[4-(4-methoxypiperidin-1-yl)phenyl]-5H,6H,7H,8H-pyrido[3,4-d]pyrimidin-2-amine as brown solid (500 mg; Crude Product).

3-[(7-[8-methyl-1H,2H,3H-pyrido[2,3-b][1,4]oxazin-7-yl]-5H,6H,8H-pyrido[3,4-d]pyrimidin-2-yl)amino]-5,6,7,8-tetrahydroquinolin-6-ol: To a stirred solution of N-[4-(4-methoxypiperidin-1-yl)phenyl]-5H,6H,7H,8H-pyrido[3,4-d]pyrimidin-2-amine (200 mg; 0.59 mmol) and tert-butyl 7-bromo-8-methyl-2H,3H-pyrido[2,3-b][1,4]oxazine-1-carboxylate (340 mg; 1.00 mmol) in 1,4-dioxane (10 mL) was added Cs₂CO₃ (500 mg; 1.52 mmol) and Pd-PEPPSI-IPentCl 2-methylpyridine (o-picoline) (60 mg; 0.07 mmol). The resulting mixture was stirred for 3 ds at 90 degrees C. under nitrogen atmosphere. The resulting mixture was concentrated under vacuum. The residue was purified by silica gel column chromatography and eluted with CH₂Cl₂/MeOH (1:1) to afford 3-[(7-[8-methyl-1H,2H,3H-pyrido[2,3-b][1,4]oxazin-7-yl]-5H,6H,8H-pyrido[3,4-d]pyrimidin-2-yl)amino]-5,6,7,8-tetrahydroquinolin-6-ol as yellow solid (90 mg; 26.0%).

N-[4-(4-methoxypiperidin-1-yl)phenyl]-7-{8-methyl-1H,2H,3H-pyrido[2,3-b][1,4]oxazin-7-yl}-5H,6H,7H,8H-pyrido[3,4-d]pyrimidin-2-amine: To a stirred solution of 3-[(7-[8-methyl-1H,2H,3H-pyrido[2,3-b][1,4]oxazin-7-yl]-5H,6H,8H-pyrido[3,4-d]pyrimidin-2-yl)amino]-5,6,7,8-tetrahydroquinolin-6-ol (90 mg; 0.15 mmol) in DCM (15 mL) was added TFA (2 mL). The resulting mixture was stirred for 2 h at room temperature. The resulting mixture was concentrated under vacuum. The residue was purified by silica gel column chromatography and eluted with PE/EtOAc CH₂Cl₂/MeOH (1:1) to afford N-[4-(4-methoxypiperidin-1-yl)phenyl]-7-{8-methyl-1H,2H,3H-pyrido[2,3-b][1,4]oxazin-7-yl}-5H,6H,7H,8H-pyrido[3,4-d]pyrimidin-2-amine as off-white solid (6 mg, 7.7%).

HPLC 95.32% purity, 6.59 min. MS: m/s=488.35 [M+H]⁺. ¹H NMR (300 MHz, DMSO-d6): 9.22 (s, 1H), 8.27 (s, 1H), 7.56 (d, J=8.9 Hz, 2H), 7.29 (s, 1H), 6.88 (d, J=9.0 Hz, 2H), 5.53 (s, 1H), 4.20 (t, J=4.2 Hz, 2H), 3.93 (s, 2H), 3.28 (s, 3H), 3.11 (d, J=6.1 Hz, 2H), 2.80 (td, J=9.8, 4.6 Hz, 4H), 2.05 (s, 3H), 1.94 (d, J=11.3 Hz, 2H), 1.63-1.49 (m, 6H).

### Example 114: Synthesis of Compound 114 7-(1-methyl-1H-pyrazol-4-yl)-N-(6-methyl-5,6,7,8-tetrahydro-1,6-naphthyridin-3-yl)-5,6,7,8-tetrahydropyrido[3,4-d]pyrimidin-2-amine

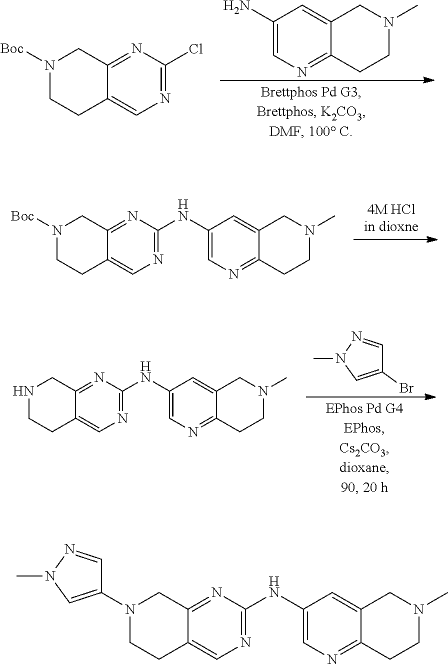

tert-butyl 2-[(6-methyl-7,8-dihydro-5H-1,6-naphthyridin-3-yl)amino]-5H,6H,8H-pyrido[3,4-d]pyrimidine-7-carboxylate: To a stirred solution of tert-butyl 2-chloro-5H,6H,8H-pyrido[3,4-d]pyrimidine-7carboxylate (1.25 g, 4.579 mmol), 6-methyl-7,8-dihydro-5H-1,6-naphthyridin3amine (915 mg, 5.533 mmol) and K₂CO₃ (1.92 g, 13.198 mmol) in DMF (25 mL, 323.044 mmol) were added BrettPhos Pd G3 (412 mg, 0.432 mmol) and BrettPhos (499 mg, 0.883 mmol) at room temperature under air atmosphere. The resulting mixture was stirred for 3 h at 100 degrees C. under argon atmosphere. The mixture was allowed to cool down to room temperature. The mixture was quenched by water. The resulting mixture was extracted with EtOAc (3×50 mL). The combined organic layers were washed with brine (3×50 mL) and dried over anhydrous Na₂SO₄. After filtration, the filtrate was concentrated under reduced pressure. The residue was purified by Prep-TLC (CH₂Cl₂/MeOH 10:1) to afford tert-butyl 2-[(6-methyl-7,8-dihydro-5H-1,6-naphthyridin-3-yl)amino]-5H,6H,8H-pyrido[3,4-d]pyrimidine-7-carboxylate as a brown solid (1.5 g, 82.63%).

6-methyl-N-[5H,6H,7H,8H-pyrido[3,4-d]pyrimidin-2-yl]-7,8-dihydro-5H-1,6-naphthyridin-3-amine: A solution of tert-butyl 2-[(6-methyl-7,8-dihydro-5H-1,6-naphthyridin-3-yl)amino]-5H,6H,8H-pyrido[3,4-d]pyrimidine-7-carboxylate (660 mg, 1.623 mmol,) in HCl (gas) in 1,4-dioxane (6 mL, 21.021 mmol) was stirred for 3 h at room temperature under nitrogen atmosphere. The resulting mixture was diluted with 10 mL of water, then adjusted to pH 6-7 with sodium bicarbonate. The resulting solution was extracted with 3×50 mL of MeOH and CH₂Cl₂ (MeOH: CH₂Cl₂=1:10). The combined organic layer was dried over anhydrous sodium sulfate and concentrated under vacuum. This result in 6-methyl-N-[5H,6H,7H,8H-pyrido[3,4-d]pyrimidin-2-yl]-7,8-dihydro-5H-1,6-naphthyridin-3-amine as a yellow solid (460 mg, 95.63%).

6-methyl-N-[7-(1-methyl-1H-pyrazol-4-yl)-5H,6H,7H,8H-pyrido[3,4-d]pyrimidin-2-yl]-5,6,7,8-tetrahydro-1,6-naphthyridin-3-amine: To a solution of 6-methyl-N-{5H,6H,7H,8H-pyrido[3,4-d]pyrimidin-2-yl}-5,6,7,8-tetrahydro-1,6-naphthyridin-3-amine (150 mg; 0.48 mmol) and 4-bromo-1-methyl-1H-pyrazole (180 mg; 1.06 mmol) in 1,4-dioxane (10 mL) were added Cs₂CO₃ (300 mg; 0.91 mmol) and Ephos (66 mg; 0.12 mmol) EPhos Pd G4 (45 mg; 0.05 mmol). After stirring for 16 h at 90 degrees, it was purified by HPLC with the following conditions: Column: XBridge Prep OBD C18 Column, 30×150 mm, 5 μm; Mobile Phase A: water (10 mmol/mL NH₄HCO₃), Mobile Phase B: ACN; Flow rate:60 mL/min.; Gradient:25 B to 55 B in 9 min.; UV 254 nm; RT1:7. This resulted in 6-methyl-N-[7-(1-methyl-1H-pyrazol-4-yl)-5H,6H,7H,8H-pyrido[3,4-d]pyrimidin-2-yl]-5,6,7,8-tetrahydro-1,6-naphthyridin-3-amine as off-white solid (9.00 mg; 4.8%).

HPLC 95.63% purity, 4.09 min. MS: m/s=377.10 [M+H]⁺. ¹H NMR (400 MHz, DMSO-d6): 9.55 (s, 1H), 8.64 (d, J=2.5 Hz, 1H), 8.31 (s, 1H), 7.85 (d, J=2.5 Hz, 1H), 7.41 (d, J=0.9 Hz, 1H), 7.28 (d, J=0.9 Hz, 1H), 3.95 (s, 2H), 3.74 (s, 3H), 3.49 (s, 2H), 3.25-3.18 (m, 2H), 2.86-2.73 (m, 4H), 2.71-2.63 (m, 2H), 2.36 (s, 3H).

### Example 115: Synthesis of Compound 115 7-(8-methyl-2,3-dihydro-1H-pyrido[2,3-b][1,4]oxazin-7-yl)-N-(6-methyl-5,6,7,8-tetrahydro-1,6-naphthyridin-3-yl)-5,6,7,8-tetrahydropyrido[3,4-d]pyrimidin-2-amine

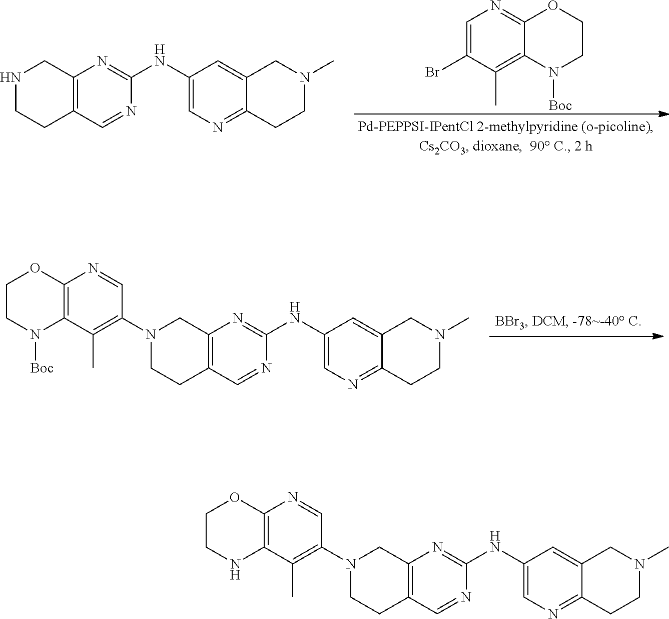 [SMILES: CN1CCc2ncc(Nc3ncc4c(n3)CNCC4)cc2C1 ; Cc1c(Br)cnc2c1N(C(=O)OC(C)(C)C)CCO2 ; Cc1c(N2CCc3cnc(Nc4cnc5c(c4)CN(C)CC5)nc3C2)cnc2c1N(C(=O)OC(C)(C)C)CCO2 ; Cc1c(N2CCc3cnc(Nc4cnc5c(c4)CN(C)CC5)nc3C2)cnc2c1NCCO2]

tert-butyl 8-methyl-7-{2-[(6-methyl-5,6,7,8-tetrahydro-1,6-naphthyridin-3-yl)amino]-5H,6H,7H,8H-pyrido[3,4-d]pyrimidin-7-yl}-1H,2H,3H-pyrido[2,3-b][1,4]oxazine-1-carboxylate: To a stirred solution of 6-methyl-N-{5H,6H,7H,8H-pyrido[3,4-d]pyrimidin-2-yl}-5,6,7,8-tetrahydro-1,6-naphthyridin-3-amine (500 mg; 1.69 mmol) and tert-butyl 7-bromo-8-methyl-1H,2H,3H-pyrido[2,3-b][1,4]oxazine-1-carboxylate (850 mg; 2.58 mmol) in 1,4-dioxane (15 mL) was added Cs₂CO₃ (1650 mg; 5.01 mmol) and Pd-PEPPSI-IPentCl 2-methylpyridine (o-picoline) (150 mg; 0.17 mmol) under nitrogen atmosphere. The resulting mixture was stirred for overnight at 90 degrees C. under nitrogen atmosphere. The resulting mixture was concentrated under vacuum. The residue was purified by silica gel column chromatography and eluted with CH₂Cl₂/MeOH (1:1) to afford tert-butyl 8-methyl-7-{2-[(6-methyl-5,6,7,8-tetrahydro-1,6-naphthyridin-3-yl)amino]-5H,6H,7H,8H-pyrido[3,4-d]pyrimidin-7-yl}-1H,2H,3H-pyrido[2,3-b][1,4]oxazine-1-carboxylate as yellow solid (470 mg; 49.2%).

6-methyl-N-(7-{8-methyl-1H,2H,3H-pyrido[2,3-b][1,4]oxazin-7-yl}-5H,6H,7H,8H-pyrido[3,4-d]pyrimidin-2-yl)-5,6,7,8-tetrahydro-1,6-naphthyridin-3-amine: To a stirred solution of tert-butyl 8-methyl-7-{2-[(6-methyl-5,6,7,8-tetrahydro-1,6-naphthyridin-3-yl)amino]-5H,6H,7H,8H-pyrido[3,4-d]pyrimidin-7-yl}-1H,2H,3H-pyrido[2,3-b][1,4]oxazine-1-carboxylate (250 mg; 0.44 mmol) in DCM (30 mL) was added BBr3 in DCM (2 mL; 2.00 mmol) at −78 degrees C. under nitrogen atmosphere. The resulting mixture was stirred for 2 h at −78 degrees C. under nitrogen atmosphere. The resulting mixture was concentrated under vacuum. The residue was purified by silica gel column chromatography and eluted with CH₂Cl₂/MeOH (1:1) to afford product. The product was purified by reverse flash chromatography with the following conditions: Column: XBridge Prep OBD C18 Column, 19*250 mm, 5 μm; Mobile Phase A: water (10 mmol/L NH₄HCO₃+0.05% NH₄OH), Mobile Phase B:ACN; Flow rate: 20 mL/min.; Gradient: 25 B to 40 B in 8 min., 254 nm; RT1:7. This resulted in 6-methyl-N-(7-{8-methyl-1H,2H,3H-pyrido[2,3-b][1,4]oxazin-7-yl}-5H,6H,7H,8H-pyrido[3,4-d]pyrimidin-2-yl)-5,6,7,8-tetrahydro-1,6-naphthyridin-3-amine as white solid (52.60 mg, 26.4%).

HPLC 98.66% purity, 2.38 min. MS: m/s=445.20 [M+H]⁺. ¹H NMR (400 MHz, DMSO-d6, ppm): 9.58 (s, 1H), 8.61 (d, J=2.5 Hz, 1H), 8.34 (s, 1H), 7.89 (d, J=2.5 Hz, 1H), 7.29 (s, 1H), 5.55-5.49 (m, 1H), 4.22-4.16 (m, 2H), 3.96 (s, 2H), 3.48 (s, 2H), 3.29 (d, J=4.8 Hz, 2H), 3.16-3.08 (m, 2H), 2.85-2.75 (m, 4H), 2.70-2.63 (m, 2H), 2.35 (s, 3H), 2.04 (s, 3H).

### Example 116: Synthesis of Compound 116 7-(5-amino-4-methylpyridin-3-yl)-N-(6-methyl-5,6,7,8-tetrahydro-1,6-naphthyridin-3-yl)-5,6,7,8-tetrahydropyrido[3,4-d]pyrimidin-2-amine

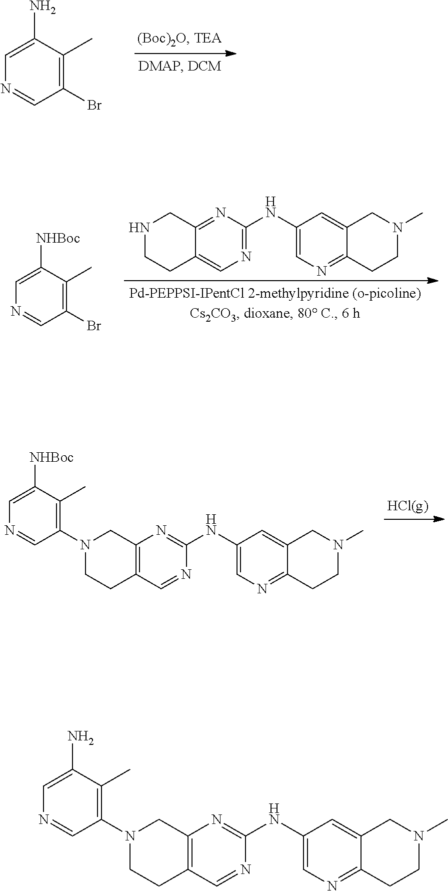

tert-butyl N-(5-bromo-4-methylpyridin-3-yl)carbamate: To a stirred solution of 5-bromo-4-methylpyridin-3-amine (200 mg, 1.016 mmol) and TEA (340 mg, 3.192 mmol) and DMAP (160 mg, 1.244 mmol) in DCM (10.00 mL, 157.300 mmol) was added Boc₂O (320 mg, 1.393 mmol) at 25 degrees C. The resulting mixture was stirred for 2 h at room temperature. This resulted in tert-butyl N-(5-bromo-4-methylpyridin-3-yl)carbamate as a yellow solid (180 mg, 41.53%).

N-(4-methyl-5-[2-[(6-methyl-7,8-dihydro-5H-1,6-naphthyridin-3-yl)amino]-5H,6H,8H-pyrido[3,4-d]pyrimidin-7-yl]pyridin-3-yl)carbamate: To a stirred solution of 6-methyl-N-[5H,6H,7H,8H-pyrido[3,4-d]pyrimidin-2-yl]-7,8-dihydro-5H-1,6-naphthyridin-3-amine (100 mg, 0.337 mmol) and tert-butyl N-(5-bromo-4-methylpyridin-3-yl)carbamate (200 mg, 0.469 mmol) in 1,4-dioxane (8 mL, 94.433 mmol) were added Pd-PEPPSI-IPentCl 2-methylpyridine (o-picoline (15 mg, 0.169 mmol) and Cs₂CO₃ (250 mg, 0.729 mmol). The resulting mixture was stirred for 6 h at 80 degrees C. under nitrogen atmosphere. This result in tert-butyl N-(4-methyl-5-[2-[(6-methyl-7,8-dihydro-5H-1,6-naphthyridin-3-yl)amino]-5H,6H,8H-pyrido[3,4-d]pyrimidin-7-yl]pyridin-3-yl)carbamate as yellow solid (70 mg, 41.28%).

N-[7-(5-amino-4-methylpyridin-3-yl)-5H,6H,8H-pyrido[3,4-d]pyrimidin-2-yl]-6-methyl-7,8-dihydro-5H-1,6-naphthyridin-3-amine: A solution of tert-butyl N-(4-methyl-5-[2-[(6-methyl-7,8-dihydro-5H-1,6-naphthyridin-3-yl)amino]-5H,6H,8H-pyrido[3,4-d]pyrimidin-7-yl]pyridin-3-yl)carbamate (64 mg, 0.127 mmol) in HCl (gas) in 1,4-dioxane (2 mL, 87.587 mmol,) and 1,4-dioxane (5 mL) was stirred for 2 h at room temperature under nitrogen atmosphere. This result in N-[7-(5-amino-4-methylpyridin-3-yl)-5H,6H,8H-pyrido[3,4-d]pyrimidin-2-yl]-6-methyl-7,8-dihydro-5H-1,6-naphthyridin-3-amine as a grey solid (9.3 mg, 16.33%).

HPLC 89.0% purity, 6.760 min., MS: m/s=403.3 [M+H]⁺. ¹H NMR (300 MHz, DMSO-d6): 9.00 (d, J=2.4 Hz, 1H), 8.46 (s, 1H), 8.29 (d, J=2.4 Hz, 1H), 7.85 (d, J=2.4 Hz, 2H), 4.52 (s, 3H), 4.18 (s, 2H), 3.62 (s, 2H), 3.27 (d, J=6.5 Hz, 5H), 2.97 (s, 4H), 2.90 (s, 2H), 2.23 (s, 3H).

### Example 117: Synthesis of Compound 117 7-(2-fluoro-6-methoxyphenyl)-N-(4-(1-methylpiperidin-4-yl)phenyl)-5,6,7,8-tetrahydropyrido[3,4-d]pyrimidin-2-amine

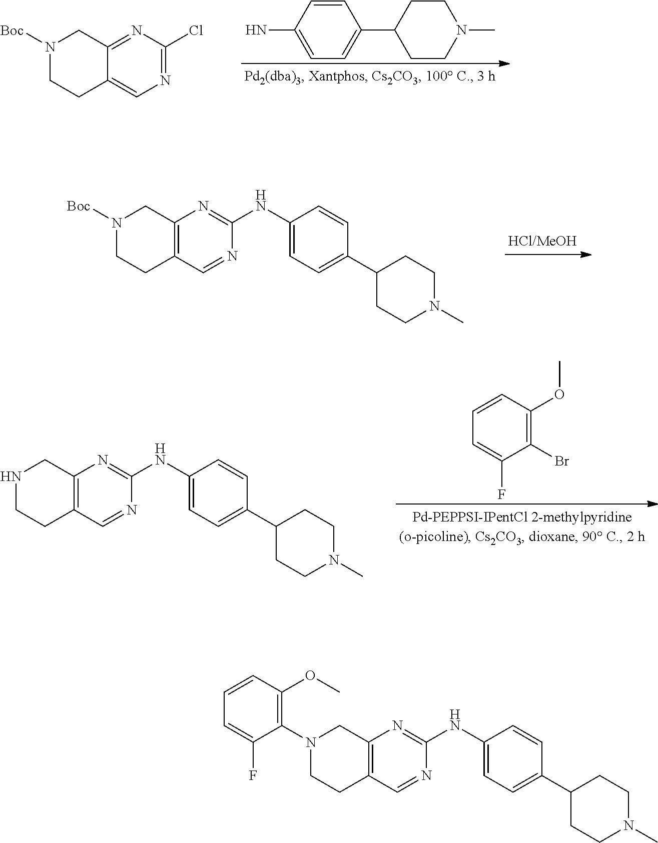

2-[[4-(1-methylpiperidin-4-yl)phenyl]amino]-5H,6H,8H-pyrido[3,4-d]pyrimidine-7-carboxylate: A solution of tert-butyl 2-chloro-5H,6H,8H-pyrido[3,4-d]pyrimidine-7-carboxylate (300 mg, 1.102 mmol),4-(1-methylpiperidin-4-yl)aniline (331 mg, 1.653 mmol), Pd2(dba)₃ (106 mg, 0.110 mmol), XantPhos (134 mg, 0.220 mmol), Cs₂CO₃ (1077 mg, 3.307 mmol) and dioxane (30 mL, 340.499 mmol) was stirred for 3 h at 90° C. under argon atmosphere. The resulting mixture was concentrated under vacuum. The residue was purified by silica gel column chromatography and eluted with CH₂Cl₂/MeOH (10:1) to afford tert-butyl 2-[[4-(1-methylpiperidin-4-yl)phenyl]amino]-5H,6H,8H-pyrido[3,4-d]pyrimidine-7-carboxylate as a yellow solid (434 mg, 91.20%).

N-[4-(1-methylpiperidin-4-yl)phenyl]-5H,6H,7H,8H-pyrido[3,4-d]pyrimidin-2-amine: A solution of tert-butyl 2-[[4-(1-methylpiperidin-4-yl)phenyl]amino]-5H,6H,8H-pyrido[3,4-d]pyrimidine-7-carboxylate (400 mg, 0.926 mmol) and HCl (gas) in 1,4-dioxane (15 mL, 26.280 mmol) was stirred for 2 h at room temperature under air atmosphere. The reaction was quenched by the addition of water. The mixture was neutralized to pH 7 with NaOH. The aqueous layer was extracted with CH₂Cl₂. The resulting mixture was concentrated under vacuum. This resulted in N-[4-(1-methylpiperidin-4-yl)phenyl]-5H,6H,7H,8H-pyrido[3,4-d]pyrimidin-2-amine as a yellow solid (240 mg, 69.84%).

7-(2-fluoro-6-methoxyphenyl)-N-[4-(1-methylpiperidin-4-yl)phenyl]-5H,6H,8H-pyrido[3,4-d]pyrimidin-2-amine: A mixture of 2-bromo-1-fluoro-3-methoxybenzene (201.32 mg, 0.933 mmol),N-[4-(1-methylpiperidin-4-yl)phenyl]-5H,6H,7H,8H-pyrido[3,4-d]pyrimidin-2-amine (173 mg, 0.466 mmol), Pd PEPPSI IPentCl (42.25 mg, 0.047 mmol), Cs₂CO₃ (303.93 mg, 0.932 mmol) and dioxane (12.00 mL, 141.649 mmol) was stirred for overnight at 100° C. under nitrogen atmosphere. The resulting mixture was concentrated under vacuum, and purified by Prep-HPLC with the following conditions (Column: XBridge Prep OBD C18 Column, 19*250 mm, 5 μm; Mobile Phase A: water (10 mmol/L NH₄HCO₃+0.05% NH₄OH), Mobile Phase B: ACN; Flow rate: 20 mL/min; Gradient: 60% B to 90% B in 8 min, Wave Length: 254 nm; RT1 (min.): 7; Number Of Runs: 1) to afford 7-(2-fluoro-6-methoxyphenyl)-N-[4-(1-methylpiperidin-4-yl)phenyl]-5H,6H,8H-pyrido[3,4-d]pyrimidin-2-amine as white solid (7.5 mg, 3.52%).

HPLC 98.1% purity, 4.280 min., MS: m/s=448.1 [M+H]⁺. ¹H NMR (400 MHz, DMSO-d6) 9.37 (s, 1H), 8.27 (s, 1H), 7.63 (d, J=8.5 Hz, 2H), 7.17-7.01 (m, 3H), 6.90-6.75 (m, 2H), 4.11 (s, 2H), 3.79 (s, 3H), 3.34 (s, 2H), 2.85 (d, J=11.0 Hz, 2H), 2.73-2.63 (m, 2H), 2.40-2.31 (m, 1H), 2.18 (s, 3H), 1.94 (t, J=11.4 Hz, 2H), 1.73-1.52 (m, 4H).

### Example 118: Synthesis of Compound 118 N-(6-methyl-5,6,7,8-tetrahydro-1,6-naphthyridin-3-yl)-7-(4-methylpyridin-3-yl)-5,6,7,8-tetrahydropyrido[3,4-d]pyrimidin-2-amine

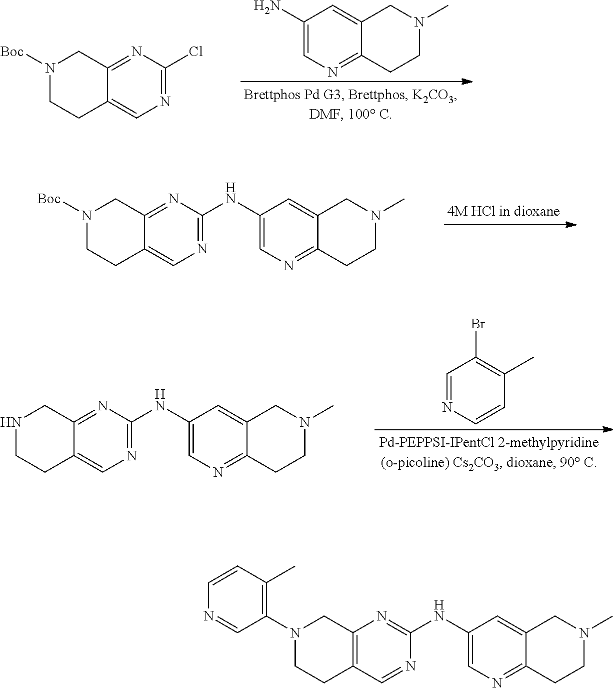

2-[(6-methyl-7,8-dihydro-5H-1,6-naphthyridin-3-yl)amino]-5H,6H,8H-pyrido[3,4-d]pyrimidine-7-carboxylate: To a stirred solution of tert-butyl 2-chloro-5H,6H,8H-pyrido[3,4-d]pyrimidine-7-carboxylate (1.25 g, 4.579 mmol), 6-methyl-7,8-dihydro-5H-1,6-naphthyridin-3-amine (915 mg, 5.533 mmol) and K₂CO₃ (1.92 g, 13.198 mmol) in DMF (25 mL, 323.044 mmol) were added BrettPhos Pd G3 (412 mg, 0.432 mmol) and BrettPhos (499 mg, 0.883 mmol) at room temperature under air atmosphere. The resulting mixture was stirred for 3 h at 100° C. under argon atmosphere. The mixture was allowed to cool down to room temperature. The resulting mixture was extracted with EtOAc (3×50 mL). The combined organic layers were washed with brine (3×50 mL) and dried over anhydrous Na₂SO₄. After filtration, the filtrate was concentrated under reduced pressure. The residue was purified by Prep-TLC (CH₂Cl₂/MeOH 10:1) to afford tert-butyl 2-[(6-methyl-7,8-dihydro-5H-1,6-naphthyridin-3-yl)amino]-5H,6H,8H-pyrido[3,4-d]pyrimidine-7-carboxylate (1.5 g, 82.63%) as a brown solid.

6-methyl-N-[5H,6H,7H,8H-pyrido[3,4-d]pyrimidin-2-yl]-7,8-dihydro-5H-1,6-naphthyridin-3-amine: A solution of tert-butyl 2-[(6-methyl-7,8-dihydro-5H-1,6-naphthyridin-3-yl)amino]-5H,6H,8H-pyrido[3,4-d]pyrimidine-7-carboxylate (596.00 mg, 1.466 mmol) in HCl (gas) in 1,4-dioxane (6.0 mL, 21.021 mmol) was stirred for 3 h at room temperature under nitrogen atmosphere. The resulting mixture was diluted with 10 mL of water, then adjusted to pH 6-7 with sodium bicarbonate. The resulting solution was extracted with 3×50 mL of MeOH and CH₂Cl₂ (MeOH: CH₂Cl₂=1:10). The combined organic layer was dried over anhydrous sodium sulfate and concentrated under vacuum. This result in 6-methyl-N-[5H,6H,7H,8H-pyrido[3,4-d]pyrimidin-2-yl]-7,8-dihydro-5H-1,6-naphthyridin-3-amine (366 mg, 80.97%) as a yellow solid.

6-methyl-N-[7-(4-methylpyridin-3-yl)-5H,6H,8H-pyrido[3,4-d]pyrimidin-2-yl]-7,8-dihydro-5H-1,6-naphthyridin-3-amine: To a stirred solution of 6-methyl-N-[5H,6H,7H,8H-pyrido[3,4-d]pyrimidin-2-yl]-7,8-dihydro-5H-1,6-naphthyridin-3-amine (100 mg, 0.324 mmol), 3-bromo-4-methylpyridine (70 mg, 0.387 mmol) and Cs₂CO₃ (200 mg, 0.583 mmol) in 1,4-dioxane (8 mL, 94.433 mmol) were added Pd-PEPPSI-IPentCl 2-methylpyridine (o-picoline (50 mg, 0.056 mmol) at room temperature under air atmosphere.

The resulting mixture was stirred for 2 h at 90 degree C. under nitrogen atmosphere.

The resulting mixture was concentrated under reduced pressure. The crude product was purified by Prep-HPLC with the following conditions (2 #SHIMADZU (HPLC-01)): Column: Column: (XBridge Prep OBD C18 Column, 30*150 mm, 5 μm; Mobile Phase A: water (10 mmol/L NH₄HCO₃), Mobile Phase B: ACN; Flow rate: 60 mL/min.; Gradient: 20% B to 50% B in 9 min., Wave Length: 254 nm; RT1 (min.): 7.2; Number Of Runs: 2). This result in 6-methyl-N-[7-(4-methylpyridin-3-yl)-5H,6H,8H-pyrido[3,4-d]pyrimidin-2-yl]-7,8-dihydro-5H-1,6-naphthyridin-3-amine (23.1 mg, 18.35%) as a white solid.

HPLC 99.8% purity, 2.402 min., MS: m/s=388.3 [M+H]⁺. ¹H NMR (400 MHz, DMSO-d6): 9.60 (s, 1H), 8.63 (d, J=2.5 Hz, 1H), 8.36 (d, J=6.5 Hz, 2H), 8.19 (d, J=4.8 Hz, 1H), 7.90 (d, J=2.5 Hz, 1H), 7.24 (d, J=4.8 Hz, 1H), 4.14 (s, 2H), 3.50 (s, 2H), 3.26 (t, J=5.7 Hz, 2H), 2.82 (t, J=5.8 Hz, 4H), 2.68 (t, J=5.9 Hz, 2H), 2.36 (s, 3H), 2.31 (s, 3H).

### Example 119: Synthesis of Compound 119 N-(6-methyl-5,6,7,8-tetrahydro-1,6-naphthyridin-3-yl)-7-(4-methylisothiazol-5-yl)-5,6,7,8-tetrahydropyrido[3,4-d]pyrimidin-2-amine

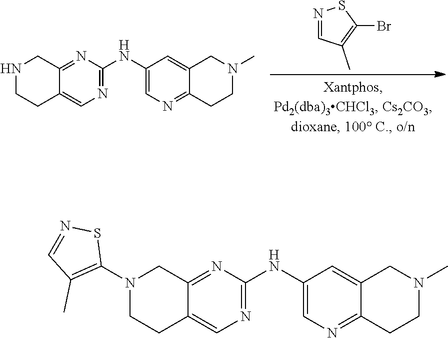

6-methyl-N-[7-(4-methyl-1,2-thiazol-5-yl)-5H,6H,8H-pyrido[3,4-d]pyrimidin-2-yl]-7,8-dihydro-5H-1,6-naphthyridin-3-amine: To a stirred solution of 6-methyl-N-[5H,6H,7H,8H-pyrido[3,4-d]pyrimidin-2-yl]-7,8-dihydro-5H-1,6-naphthyridin-3-amine (200 mg, 0.661 mmol), 5-bromo-4-methyl-1,2-thiazole (144 mg, 0.768 mmol) and Cs₂CO₃ (461 mg, 1.344 mmol) in dioxane (8 mL) were added Pd-PEPPSI-IPentCl 2-methylpyridine (o-picoline (60 mg, 0.068 mmol) at room temperature under air atmosphere. The resulting mixture was stirred for 5 h at 90° C. under argon atmosphere. The mixture was allowed to cool down to room temperature. The resulting mixture was concentrated under reduced pressure. The residue was purified by Prep-TLC (CH₂Cl₂/MeOH 8:1). The crude product (in order to dissolve, add a few drops of formic acid) was purified by Prep-HPLC with the following conditions (2 #SHIMADZU (HPLC-01):(Column: XBridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 20% B to 50% B in 8 min., Wave Length: 254 nm; RTT (min.): 7.46; Number Of Runs: 2); to afford 6-methyl-N-[7-(4-methyl-1,2-thiazol-5-yl)-5H,6H,8H-pyrido[3,4-d]pyrimidin-2-yl]-7,8-dihydro-5H-1,6-naphthyridin-3-amine; formic acid (24.6 mg, 8.18%) as a yellow solid.

HPLC 99.6% purity, 6.201 min., MS: m/s=394.2 [M+H]⁺. ¹H NMR (400 MHz, DMSO-d6): 9.64 (s, 1H), 8.65 (d, J=2.5 Hz, 1H), 8.37 (s, 1H), 8.19 (s, 1H), 8.14 (s, 1H), 7.84 (d, J=2.5 Hz, 1H), 4.27 (s, 2H), 3.50 (q, J=5.7, 4.5 Hz, 4H), 2.84 (q, J=6.2 Hz, 4H), 2.70 (s, 2H), 2.37 (s, 3H), 2.21 (s, 3H).

### Example 120: Synthesis of Compound 120 7-(2-fluoro-6-methoxyphenyl)-N-(6-methyl-5,6,7,8-tetrahydro-1,6-naphthyridin-3-yl)-5,6,7,8-tetrahydropyrido[3,4-d]pyrimidin-2-amine

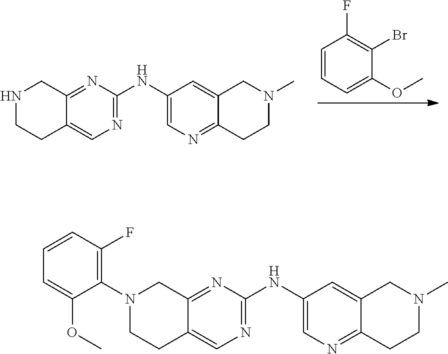

N-[7-(2-fluoro-6-methoxyphenyl)-5H,6H,8H-pyrido[3,4-d]pyrimidin-2-yl]-6-methyl-7,8-dihydro-5H-1,6-naphthyridin-3-amine (22.6 mg, 13.46%): To a stirred solution of 6-methyl-N-[5H,6H,7H,8H-pyrido[3,4-d]pyrimidin-2-yl]-7,8-dihydro-5H-1,6-naphthyridin-3-amine (120 mg, 0.397 mmol), 2-bromo-1-fluoro-3-methoxybenzene (166 mg, 0.769 mmol) and Cs₂CO₃ (264 mg, 0.770 mmol) in dioxane (0.5 mL, 5.902 mmol) were added Pd-PEPPSI-IPentCl 2-methylpyridine (o-picoline (34 mg, 0.038 mmol) and XantPhos (4 mg, 0.007 mmol) at room temperature under air atmosphere. The resulting mixture was stirred for 16 h at 90° C. under argon atmosphere. The mixture was allowed to cool down to room temperature. The resulting mixture was concentrated under reduced pressure. The residue was purified by Prep-TLC (CH₂Cl₂/MeOH 10:1). The resulting mixture was concentrated under reduced pressure. The residue was purified by reverse flash chromatography with the following conditions: column, C18 silica gel; mobile phase A: NH₄HCO₃ (0.05%) in water, mobile phase B: ACN, 51% B; detector, UV 200 nm. to afford N-[7-(2-fluoro-6-methoxyphenyl)-5H,6H,8H-pyrido[3,4-d]pyrimidin-2-yl]-6-methyl-7,8-dihydro-5H-1,6-naphthyridin-3-amine (22.6 mg, 13.46%) as off-white solid.

HPLC 99.2% purity, 3.283 min., MS: m/s=394.2 [M+H]⁺. ¹H NMR (400 MHz, DMSO-d6): 9.56 (s, 1H), 8.61 (d, J=2.5 Hz, 1H), 8.32 (s, 1H), 7.89 (d, J=2.4 Hz, 1H), 7.11 (td, J=8.4, 6.3 Hz, 1H), 6.91-6.76 (m, 2H), 4.13 (s, 2H), 3.79 (s, 3H), 3.48 (s, 2H), 3.31 (s, 2H), 2.82 (t, J=5.9 Hz, 2H), 2.72 (t, J=5.5 Hz, 2H), 2.66 (d, J=6.2 Hz, 2H), 2.35 (s, 3H).

### Example 121: Synthesis of Compound 121 2-(azetidin-1-yl)-N-(4-((7-(8-chloro-2,3-dihydro-1H-pyrido[2,3-b][1,4]oxazin-7-yl)-5,6,7,8-tetrahydropyrido[3,4-d]pyrimidin-2-yl)amino)-2-methylphenyl)acetamide

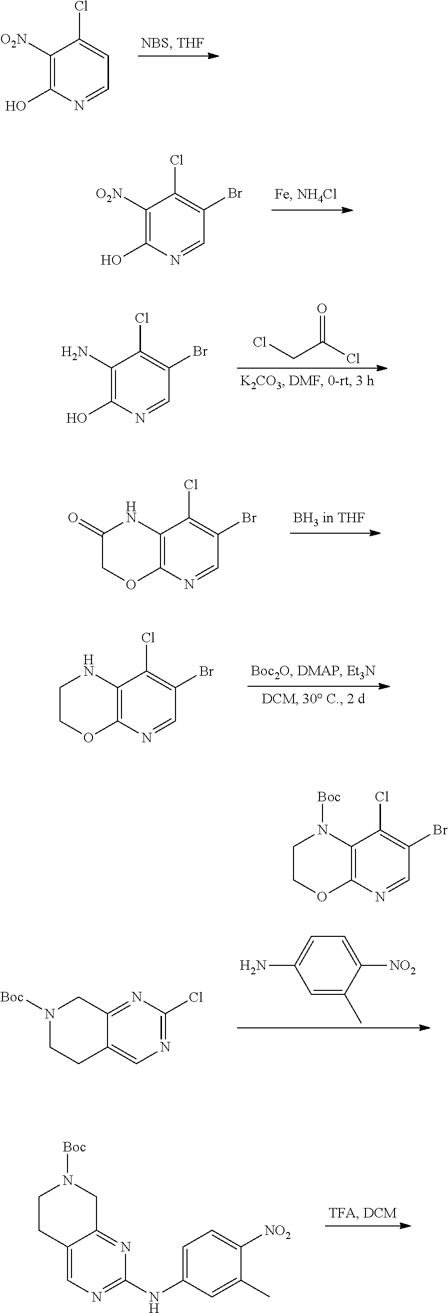

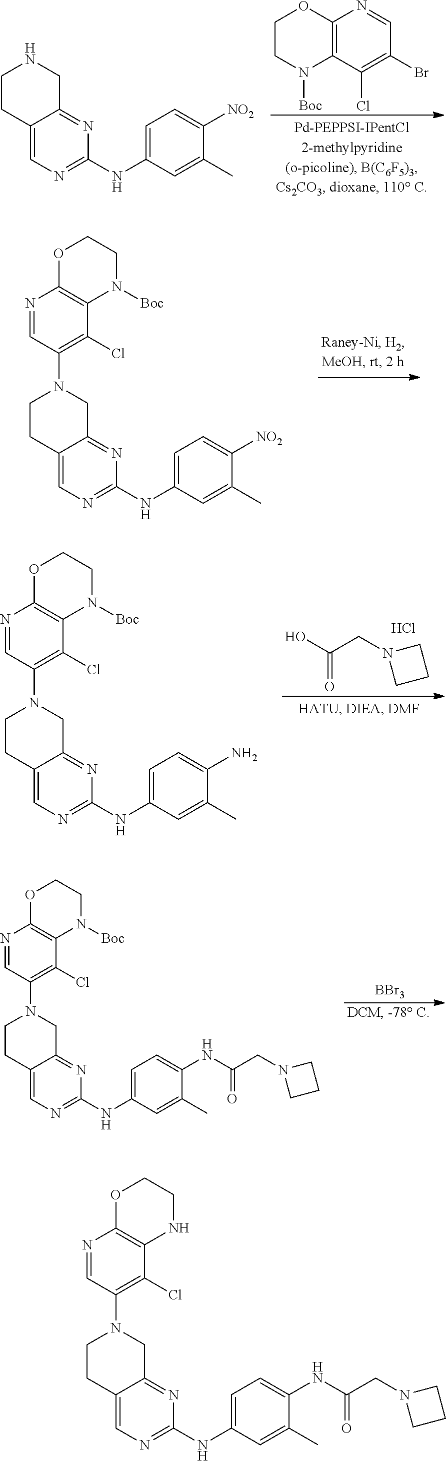

5-bromo-4-chloro-3-nitropyridin-2-ol: To a stirred solution of 4-chloro-3-nitropyridin-2-ol (75 g; 408.21 mmol) in THF (700 mL) was added NBS (89.88 g; 489.85 mmol) in portions at room temperature. The resulting mixture was stirred for overnight at room temperature. The resulting mixture was diluted with water (200 mL), extracted with EtOAc (3×100 mL). The combined organic layers were washed with brine (150 mL) and dried over anhydrous Na₂SO₄. The resulting mixture was concentrated under reduced pressure. The residue was purified by slurry with PE:EA=3:1. The solid was collected to afford 5-bromo-4-chloro-3-nitropyridin-2-ol (110 g; Crude Product).

3-amino-5-bromo-4-chloropyridin-2-ol: To a stirred solution of 5-bromo-4-chloro-3-nitropyridin-2-ol (75 g; 270.15 mmol) and NH₄Cl (60.84 g; 1080.62 mmol) in water (500 mL) and THF (900 mL) was added Fe (79.62 g; 1350.77 mmol) and in portions at room temperature. The resulting mixture was stirred for 1 h at 70 degree C. The resulting mixture was filtered and the filter cake was washed with THF (1000 mL). The resulting mixture was extracted with EtOAc (100 mL*3). The combined organic layers were washed with brine (300 mL) and dried over anhydrous Na₂SO₄. After filtration, the filtrate was concentrated under reduced pressure to afford 3-amino-5-bromo-4-chloropyridin-2-ol (70 g; Crude Product).

7-bromo-8-chloro-1H,2H,3H-pyrido[2,3-b][1,4]oxazin-2-one: To a stirred solution of 3-amino-5-bromo-4-chloropyridin-2-ol (60 g; 228.34 mmol) and K₂CO₃ (99.44 g; 685.02 mmol) in DMF (720 mL) was added 2-chloroacetyl chloride (47.79 mL; 570.85 mmol) in portions at 0 degree C. under argon atmosphere. The resulting mixture was stirred for 6 h at room temperature and for 1 h at 40 degrees. The resulting mixture was diluted with water (3000 mL). The resulting mixture was filtered and the filter cake was washed with water (1000 mL). The filter cake was concentrated under reduced pressure to afford 7-bromo-8-chloro-1H,2H,3H-pyrido[2,3-b][1,4]oxazin-2-one (40.00 g; Crude Product).

7-bromo-8-chloro-1H,2H,3H-pyrido[2,3-b][1,4]oxazine: A solution of 7-bromo-8-chloro-1H,2H,3H-pyrido[2,3-b][1,4]oxazin-2-one (2.70 g; 10.07 mmol) in BH3 in THF (350 mL; 350.00 mmol) was stirred for 2 h at 40 degree C. under N₂ atmosphere. The resulting mixture was extracted with EtOAc (100 mL*3) and quenched with water (200 mL). The combined organic layers were washed with brine (150 mL). The residue was purified by silica gel column chromatography and eluted with PE/EtOAc (3:1) to afford 7-bromo-8-chloro-1H,2H,3H-pyrido[2,3-b][1,4]oxazine (2.50 g; 99.46%).

tert-butyl 7-bromo-8-chloro-1H,2H,3H-pyrido[2,3-b][1,4]oxazine-1-carboxylate: To a stirred solution of 7-bromo-8-chloro-1H,2H,3H-pyrido[2,3-b][1,4]oxazine (2.50 g; 10.02 mmol) and di-tert-butyl dicarbonate (6.91 g; 30.06 mmol) in dichloromethane (30 mL) were added triethylamine (4.40 mL; 30.06 mmol) and N,N-dimethylpyridin-4-amine (0.26 g; 2.00 mmol) in portions at room temperature under argon atmosphere. The resulting mixture was stirred for 4 h at 30 degree C. under argon atmosphere. The resulting mixture was concentrated under reduced pressure. The residue was purified by silica gel column and eluted with 15% EtOAc in PE to afford tert-butyl 7-bromo-8-chloro-1H,2H,3H-pyrido[2,3-b][1,4]oxazine-1-carboxylate (3.50 g; 99.67%).

tert-butyl 2-[(3-methyl-4-nitrophenyl)amino]-5H,6H,7H,8H-pyrido[3,4-d]pyrimidine-7-carboxylate: To a stirred mixture of tert-butyl 2-chloro-5H,6H,7H,8H-pyrido[3,4-d]pyrimidine-7-carboxylate (3 g; 10.84 mmol) and 3-methyl-4-nitroaniline (590 g; 3761.39 mmol) in 1,4-dioxane (20 mL) were added X-PHOS (290 g; 577.91 mmol), K₂CO₃ (1261 g; 8668.23 mmol) and XPhos Pd G3 (2 g; 2.25 mmol) in portions at 25 degrees C. The resulting mixture was stirred for 2 h at 100 degrees C. under nitrogen atmosphere. The resulting mixture was concentrated under vacuum. The residue was purified by silica gel column chromatography and eluted with PE:EA=7:3 to afford tert-butyl 2-[(3-methyl-4-nitrophenyl)amino]-5H,6H,7H,8H-pyrido[3,4-d]pyrimidine-7-carboxylate as brown solid (3.50 g; 79.0%).

N-(3-methyl-4-nitrophenyl)-5H,6H,7H,8H-pyrido[3,4-d]pyrimidin-2-amine: To a stirred mixture of tert-butyl 2-[(3-methyl-4-nitrophenyl)amino]-5H,6H,7H,8H-pyrido[3,4-d]pyrimidine-7-carboxylate (3.50 g; 9.08 mmol) in DCM (30.00 mL) was added TFA (7.00 mL) in portions at 25 degrees C. The resulting mixture was stirred for 2 h at 25 degrees C. The resulting mixture was concentrated under vacuum. Purification: The reaction was diluted with NaHCO₃ (aq.) at 25 degrees C. The resulting mixture was extracted with EA (3×80 mL). The combined organic layers were washed with brine and dried over anhydrous MgSO₄. After filtration, the filtrate was concentrated under reduced pressure to afford N-(3-methyl-4-nitrophenyl)-5H,6H,7H,8H-pyrido[3,4-d]pyrimidin-2-amine as yellow solid (2.60 g; Crude Product).

tert-butyl 8-chloro-7-{2-[(3-methyl-4-nitrophenyl)amino]-5H,6H,7H,8H-pyrido[3,4-d]pyrimidin-7-yl}-1H,2H,3H-pyrido[2,3-b][1,4]oxazine-1-carboxylate: To a stirred mixture of N-(3-methyl-4-nitrophenyl)-5H,6H,7H,8H-pyrido[3,4-d]pyrimidin-2-amine (1.55 g; 4.42 mmol) and tert-butyl 7-bromo-8-chloro-1H,2H,3H-pyrido[2,3-b][1,4]oxazine-1-carboxylate (1.86 g; 5.31 mmol) in 1,4-dioxane (60 mL) were added Pd-PEPPSI-IPentCl 2-methylpyridine (o-picoline) (0.39 g; 0.44 mmol) and Cs₂CO₃ (3.03 g; 8.84 mmol) and tris(2,3,4,5,6-pentafluorophenyl)borane (0.46 g; 0.88 mmol) at room temperature. The mixture was stirred for 16 h at 110 degrees C. The resulting mixture was concentrated under reduced pressure and purified by silica gel column chromatography and eluted with PE/EtOAc (1:1) to afford tert-butyl 8-chloro-7-{2-[(3-methyl-4-nitrophenyl)amino]-5H,6H,7H,8H-pyrido[3,4-d]pyrimidin-7-yl}-1H,2H,3H-pyrido[2,3-b][1,4]oxazine-1-carboxylate as yellow solid (1.75 g; 53.2%).

tert-butyl 7-{2-[(4-amino-3-methylphenyl)amino]-5H,6H,7H,8H-pyrido[3,4-d]pyrimidin-7-yl}-8-chloro-1H,2H,3H-pyrido[2,3-b][1,4]oxazine-1-carboxylate: To a stirred mixture of tert-butyl 8-chloro-7-{2-[(3-methyl-4-nitrophenyl)amino]-5H,6H,7H,8H-pyrido[3,4-d]pyrimidin-7-yl}-1H,2H,3H-pyrido[2,3-b][1,4]oxazine-1-carboxylate (450 mg; 0.81 mmol) and Fe (238.77 mg; 4.06 mmol) in EtOH (10 mL) were added NH₄Cl (219.44 mg; 4.06 mmol) and H₂O (2.00 mL) in portions at 25 degrees C. The resulting mixture was stirred for 1 h at 80 degrees C. under nitrogen atmosphere. The resulting mixture was filtered and the filter cake was washed with MeOH. After filtration, the filtrate was concentrated under reduced pressure. This resulted in tert-butyl 7-{2-[(4-amino-3-methylphenyl)amino]-5H,6H,7H,8H-pyrido[3,4-d]pyrimidin-7-yl}-8-chloro-1H,2H,3H-pyrido[2,3-b][1,4]oxazine-1-carboxylate as yellow solid (400 mg; 87.1%).

tert-butyl 7-[2-({4-[2-(azetidin-1-yl)acetamido]-3-methylphenyl}amino)-5H,6H,7H,8H-pyrido[3,4-d]pyrimidin-7-yl]-8-chloro-1H,2H,3H-pyrido[2,3-b][1,4]oxazine-1-carboxylate: To a stirred mixture of tert-butyl 7-{2-[(4-amino-3-methylphenyl)amino]-5H,6H,7H,8H-pyrido[3,4-d]pyrimidin-7-yl}-8-chloro-1H,2H,3H-pyrido[2,3-b][1,4]oxazine-1-carboxylate (200 mg; 0.31 mmol) and in DMF (6 mL) were added HATU (373.04 mg; 0.93 mmol) and DIEA (84.50 mg; 0.62 mmol) at room temperature. The mixture was stirred for 1 h at room temperature. The resulting mixture was quenched by water and extracted with EtOAc. The combined organic layers were washed with brine and dried over anhydrous Na₂SO₄. After filtration, the filtrate was concentrated under reduced pressure. The resulting mixture was concentrated under reduced pressure and purified by silica gel column chromatography and eluted with PE/EtOAc (1:1) to afford tert-butyl 7-[2-({4-[2-(azetidin-1-yl)acetamido]-3-methylphenyl}amino)-5H,6H,7H,8H-pyrido[3,4-d]pyrimidin-7-yl]-8-chloro-1H,2H,3H-pyrido[2,3-b][1,4]oxazine-1-carboxylate as brown oil (180 mg; 74.3%).

2-(azetidin-1-yl)-N-{4-[(7-{8-chloro-1H,2H,3H-pyrido[2,3-b][1,4]oxazin-7-yl}-5H,6H,7H,8H-pyrido[3,4-d]pyrimidin-2-yl)amino]-2-methylphenyl}acetamide: To a stirred mixture of tert-butyl 7-[2-({4-[2-(azetidin-1-yl)acetamido]-3-methylphenyl}amino)-5H,6H,7H,8H-pyrido[3,4-d]pyrimidin-7-yl]-8-chloro-1H,2H,3H-pyrido[2,3-b][1,4]oxazine-1-carboxylate (170 mg; 0.24 mmol) in DCM (5 mL) was added BBr₃ in DCM (1.00 mL; 1.00 mmol) dropwise at −78 degrees C. The mixture was stirred for 1 h at −78 degrees C. The resulting mixture was concentrated under reduced pressure. The residue was purified by Prep-HPLC (Column: XBridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 24% B to 54% B in 9 min., Length: 254 nm; RT1 (min.): 7; Number Of Runs: 2) to afford 2-(azetidin-1-yl)-N-{4-[(7-{8-chloro-1H,2H,3H-pyrido[2,3-b][1,4]oxazin-7-yl}-5H,6H,7H,8H-pyrido[3,4-d]pyrimidin-2-yl)amino]-2-methylphenyl}acetamide as white solid (15.60 mg; 11.6%).

HPLC 93.31% purity, 2.88 min., MS: m/s=521.25 [M+H]⁺. ¹H NMR (300 MHz, DMSO-d6): 9.43 (s, 1H), 9.07 (s, 1H), 8.32 (s, 1H), 7.59-7.52 (m, 2H), 7.41-7.29 (m, 2H), 6.12 (s, 1H), 4.24 (t, J=4.2 Hz, 2H), 4.07 (s, 2H), 3.33 (d, J=14.0 Hz, 4H), 3.21 (m, 2H), 3.17 (s, 2H), 2.81 (s, 2H), 2.86 (s, 2H), 2.17 (s, 3H), 2.05 (p, J=6.8 Hz, 2H).

### Example 122: Synthesis of Compound 122 N-(4-((6-(8-chloro-2,3-dihydro-1H-pyrido[2,3-b][1,4]oxazin-7-yl)-5,6,7,8-tetrahydro-2,6-naphthyridin-3-yl)amino)-2-methylphenyl)-2-morpholinoacetamide

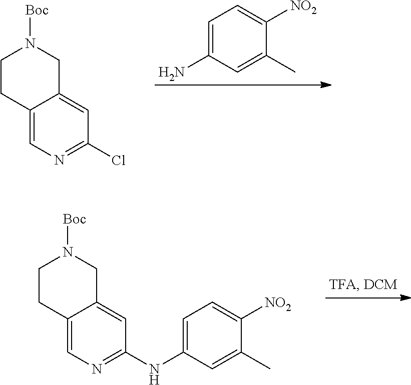 [SMILES: CC(C)(C)OC(=O)N1CCc2cnc(Cl)cc2C1 ; Cc1cc(N)ccc1[N+](=O)[O-] ; Cc1cc(Nc2cc3c(cn2)CCN(C(=O)OC(C)(C)C)C3)ccc1[N+](=O)[O-]]

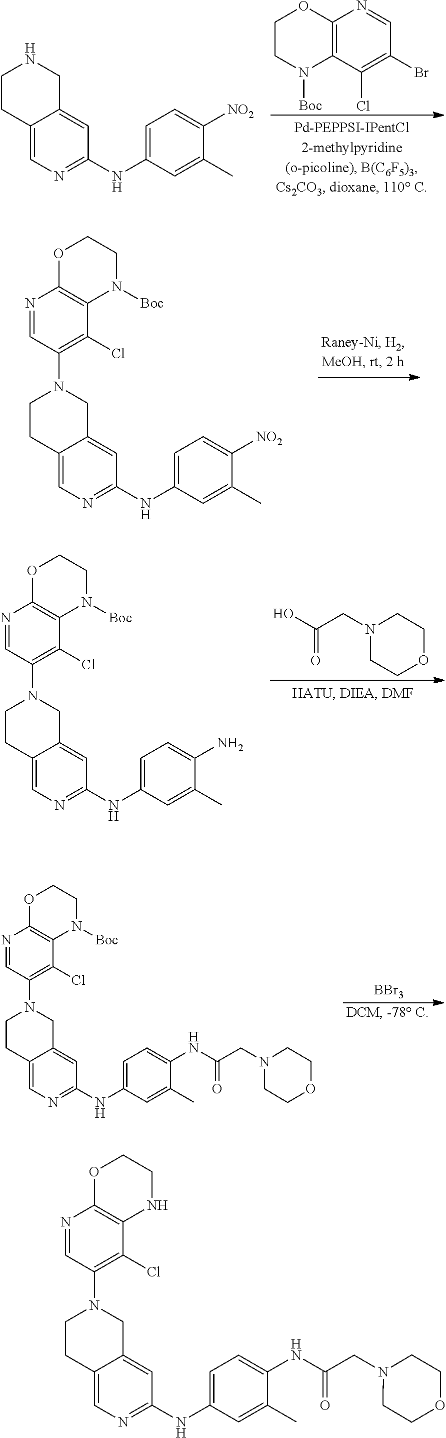

tert-butyl 7-[(3-methyl-4-nitrophenyl)amino]-1,2,3,4-tetrahydro-2,6-naphthyridine-2-carboxylate: To a stirred solution of tert-butyl 7-chloro-1,2,3,4-tetrahy dro-2,6-naphthyridine-2-carboxylate (400 mg; 1.46 mmol) and 3-methyl-4-nitroaniline (271.84 mg; 1.75 mmol) in 1,4-dioxane (5 mL) was added X-PHOS (146.44 mg; 0.29 mmol), Xphos Pd G3 (130.01 mg; 0.15 mmol) and K₂CO₃ (423.63 mg; 2.92 mmol) in portion at room temperature. The resulting mixture was stirred for 3 h at 100 degrees C. under nitrogen atmosphere. The resulting mixture was concentrated under reduced pressure. The residue was purified by silica gel column chromatography and eluted with PE/EA (1:1) to afford tert-butyl 7-[(3-methyl-4-nitrophenyl)amino]-1,2,3,4-tetrahydro-2,6-naphthyridine-2-carboxylate as yellow solid (420 mg; 74.46%).

N-(3-methyl-4-nitrophenyl)-5,6,7,8-tetrahydro-2,6-naphthyridin-3-amine: To a stirred solution of tert-butyl 7-[(3-methyl-4-nitrophenyl)amino]-1,2,3,4-tetrahydro-2,6-naphthyridine-2-carboxylate (420 mg; 1.09 mmol) in DCM (15 mL) was added TFA (3 mL) in portion at room temperature. After stirring for 1 h at room temperature, the mixture was basified to pH=7 with saturated NaHCO₃ (aq.). The resulting mixture was extracted with EtOAc. The combined organic layers were washed with NaCl (aq.) and dried over anhydrous Na₂SO₄. After filtration, the filtrate was concentrated under reduced pressure to afford N-(3-methyl-4-nitrophenyl)-5,6,7,8-tetrahydro-2,6-naphthyridin-3-amine as yellow solid (230 mg; Crude Product).

tert-butyl 8-chloro-7-{7-[(3-methyl-4-nitrophenyl)amino]-1,2,3,4-tetrahydro-2,6-naphthyridin-2-yl}-1H,2H,3H-pyrido[2,3-b][1,4]oxazine-1-carboxylate: To a stirred solution of N-(3-methyl-4-nitrophenyl)-5,6,7,8-tetrahydro-2,6-naphthyridin-3-amine (230 mg; 0.69 mmol) and tert-butyl 7-bromo-8-chloro-1H,2H,3H-pyrido[2,3-b][1,4]oxazine-1-carboxylate (302.47 mg; 0.82 mmol) in 1,4-dioxane (10 mL) was added Pd-PEPPSI-IPentCl 2-methylpyridine (o-picoline) (60.63 mg; 0.07 mmol) and Cs₂CO₃ (470.25 mg; 1.37 mmol) in portion at room temperature. The resulting mixture was stirred for 16 h at 110 degrees C. under nitrogen atmosphere. The resulting mixture was concentrated under reduced pressure. The residue was purified by silica gel column chromatography and eluted with PE/EA (1:1) to afford tert-butyl 8-chloro-7-{7-[(3-methyl-4-nitrophenyl)amino]-1,2,3,4-tetrahydro-2,6-naphthyridin-2-yl}-1H,2H,3H-pyrido[2,3-b][1,4]oxazine-1-carboxylate as yellow solid (140 mg; 30.75%).

tert-butyl 7-{7-[(4-amino-3-methylphenyl)amino]-1,2,3,4-tetrahydro-2,6-naphthyridin-2-yl}-8-chloro-1H,2H,3H-pyrido[2,3-b][1,4]oxazine-1-carboxylate: To a stirred solution of tert-butyl 8-chloro-7-{7-[(3-methyl-4-nitrophenyl)amino]-1,2,3,4-tetrahydro-2,6-naphthyridin-2-yl}-1H,2H,3H-pyrido[2,3-b][1,4]oxazine-1-carboxylate (140 mg; 0.21 mmol) and Fe (62.13 mg; 1.05 mmol) in EtOH (3 mL) and H₂O (2.00 mL) was added NH₄Cl (47.48 mg; 0.84 mmol) in portion at room temperature. After stirring for 2 h at 80 degrees C., the resulting mixture was concentrated under reduced pressure. The residue was purified by silica gel column chromatography and eluted with PE/EA (1:100) to afford tert-butyl 7-{7-[(4-amino-3-methylphenyl)amino]-1,2,3,4-tetrahydro-2,6-naphthyridin-2-yl}-8-chloro-1H,2H,3H-pyrido[2,3-b][1,4]oxazine-1-carboxylate as yellow solid (100 mg; 86.34%).

tert-butyl 8-chloro-7-[7-({3-methyl-4-[2-(morpholin-4-yl)acetamido]phenyl}amino)-1,2,3,4-tetrahydro-2,6-naphthyridin-2-yl]-1H,2H,3H-pyrido[2,3-b][1,4]oxazine-1-carboxylate: To a stirred solution of tert-butyl 7-{7-[(4-amino-3-methylphenyl)amino]-1,2,3,4-tetrahydro-2,6-naphthyridin-2-yl}-8-chloro-1H,2H,3H-pyrido[2,3-b][1,4]oxazine-1-carboxylate (100 mg; 0.18 mmol) and in DMF (7 mL) was added HATU (145.69 mg; 0.36 mmol) and DIEA (74.26 mg; 0.55 mmol) in portion at room temperature. The resulting mixture was stirred for 3 h at 40 degrees C. under nitrogen atmosphere. The resulting mixture was concentrated under reduced pressure. The residue was purified by silica gel column chromatography and eluted with CH₂Cl₂/MeOH (10:1) to afford tert-butyl 8-chloro-7-[7-({3-methyl-4-[2-(morpholin-4-yl)acetamido]phenyl}amino)-1,2,3,4-tetrahydro-2,6-naphthyridin-2-yl]-1H,2H,3H-pyrido[2,3-b][1,4]oxazine-1-carboxylate as yellow solid (70 mg; 56.77%).

N-{4-[(6-{8-chloro-1H,2H,3H-pyrido[2,3-b][1,4]oxazin-7-yl}-5,6,7,8-tetrahydro-2,6-naphthyridin-3-yl)amino]-2-methylphenyl}-2-(morpholin-4-yl)acetamide: To a stirred mixture of tert-butyl 8-chloro-7-[7-({3-methyl-4-[2-(morpholin-4-yl)acetamido]phenyl}amino)-1,2,3,4-tetrahydro-2,6-naphthyridin-2-yl]-1H,2H,3H-pyrido[2,3-b][1,4]oxazine-1-carboxylate (70 mg; 0.10 mmol) in DCM (4 mL) was added BBr₃ in DCM (0.40 mL; 0.40 mmol) in portion at −78 degrees C. The resulting mixture was stirred for 1 h at −78 degrees C. under nitrogen atmosphere. The resulting mixture was concentrated under reduced pressure. The crude product was purified by Prep-HPLC with the following conditions: Column: XBridge Prep OBD C18 Column, 19*250 mm, 5 μm; Mobile Phase A: water (10 mmol/L NH₄HCO₃+0.1% NH₄OH), Mobile Phase B: MeOH. HPLC; Flow rate: 20 mL/min.; Gradient: 75% B to 80% B in 8 min., 80% B; Wave Length: 254 nm; RT1 (min.): 7; Number Of Runs: 2, to afford N-{4-[(6-{8-chloro-1H,2H,3H-pyrido[2,3-b][1,4]oxazin-7-yl}-5,6,7,8-tetrahydro-2,6-naphthyridin-3-yl)amino]-2-methylphenyl}-2-(morpholin-4-yl)acetamide as white solid (12.50 mg; 21.73%).

HPLC 98.78% purity, 4.13 min., MS: m/s=550.15 [M+H]⁺. ¹H NMR (300 MHz, DMSO-d6): 9.21 (s, 1H), 8.75 (s, 1H), 7.99 (s, 1H), 7.44 (d, J=4.8 Hz, 3H), 7.29 (s, 1H), 6.58 (s, 1H), 6.10 (s, 1H), 4.24 (s, 2H), 4.10 (s, 2H), 3.71-3.62 (m, 4H), 3.47 (s, 2H), 3.24 (s, 2H), 3.11 (s, 2H), 2.83 (s, 2H), 2.59-2.52 (m, 4H), 2.19 (s, 3H).

### Example 123: Synthesis of Compound 123 3-methyl-1-(4-((7-(8-methyl-2,3-dihydro-1H-pyrido[2,3-b][1,4]oxazin-7-yl)-5,6,7,8-tetrahydropyrido[3,4-d]pyrimidin-2-yl)amino)benzyl)azetidin-3-ol

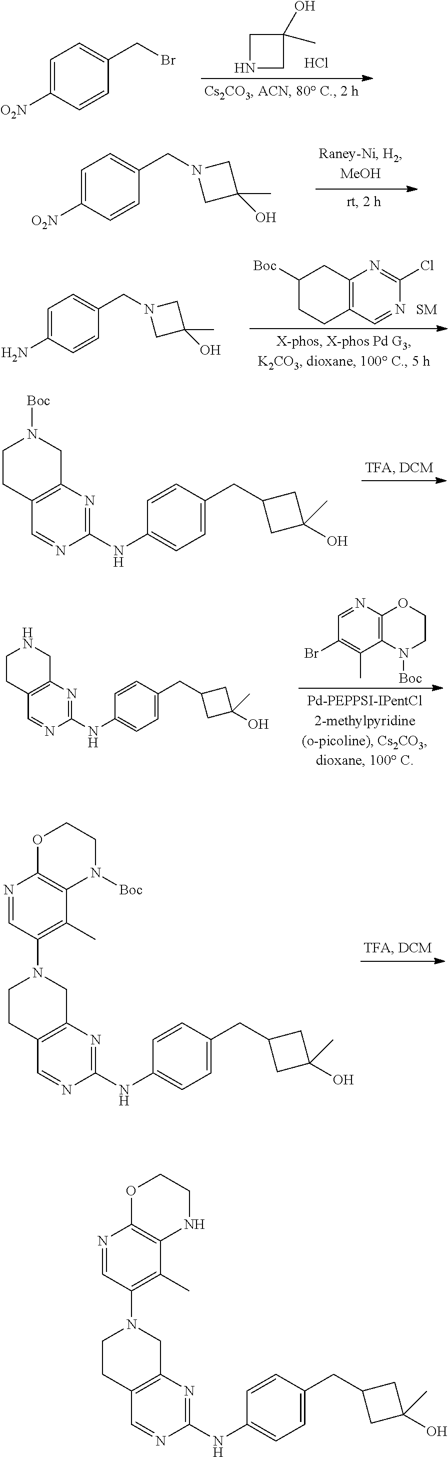

3-methyl-1-[(4-nitrophenyl)methyl]azetidin-3-ol: To a solution of 1-(bromomethyl)-4-nitrobenzene (2 g; 8.79 mmol) and 3-methylazetidin-3-ol hydrochloride (1.14 g; 8.79 mmol) in ACN (80 mL) was added Cs₂CO₃ (9.05 g; 26.38 mmol) at room temperature. The mixture stirred for 2 h at room temperature. The mixture was concentrated under reduced pressure. The residue was purified by silica gel column chromatography and eluted with PE/EtOAc (1:1) to afford 3-methyl-1-[(4-nitrophenyl)methyl]azetidin-3-ol (1.55 g; 78.03%).

1-[(4-aminophenyl)methyl]-3-methylazetidin-3-ol: To a solution of 3-methyl-1-[(4-nitrophenyl)methyl]azetidin-3-ol (1.45 g; 6.42 mmol) in MeOH (20 mL) was added Raney-Ni (1.10 g; 1.28 mmol) at room temperature. The mixture stirring at room temperature for 2 h under H₂ atmosphere. The resulting mixture was filtered and the filter cake was washed with MeOH (50 mL), the filtrate was concentrated under reduced pressure to afford 1-[(4-aminophenyl)methyl]-3-methylazetidin-3-ol (1.20 g; Crude Product).

tert-butyl2-({4-[(3-hydroxy-3-methylazetidin-1-yl)methyl]phenyl}amino)-5H,6H,7H,8H-pyrido[3,4-d]pyrimidine-7-carboxylate: To a stirred mixture of tert-butyl 2-chloro-5H,6H,7H,8H-pyrido[3,4-d]pyrimidine-7-carboxylate (400 mg; 1.48 mmol) and 1-[(4-aminophenyl)methyl]-3-methylazetidin-3-ol (296.81 mg; 1.48 mmol) in 1,4-dioxane (18 mL) were added Xphos Pd G3 (125.53 mg; 0.15 mmol), Xantphos (173.35 mg; 0.30 mmol) and Cs₂CO₃ (1017.23 mg; 2.97 mmol) at room temperature. After stirring for 3 h at 100 degrees C. under argon atmosphere, the mixture was concentrated under reduced pressure. The residue was purified by silica gel column chromatography and eluted with DCM/MeOH (11:1) to afford tert-butyl 2-({4-[(3-hydroxy-3-methylazetidin-1-yl)methyl]phenyl}amino)-5H,6H,7H,8H-pyrido[3,4-d]pyrimidine-7-carboxylate (280 mg; 40.66%).

3-methyl-1-{[4-({5H,6H,7H,8H-pyrido[3,4-d]pyrimidin-2-yl}amino)phenyl]methyl}azetidin-3-ol: To a stirred mixture of tert-butyl 2-({4-[(3-hydroxy-3-methylazetidin-1-yl)methyl]phenyl}amino)-5H,6H,7H,8H-pyrido[3,4-d]pyrimidine-7-carboxylate (270 mg; 0.58 mmol) in 4N HCl in EA (10 mL) at room temperature. After stirring for 1 h at room temperature, the mixture was concentrated under reduced pressure. The reaction was quenched with H₂O/ice water. The mixture was basified to pH=14 with NaOH. The resulting mixture was extracted with CH₂Cl₂ (10 mL*3) and quenched with water. The combined organic layers were washed with brine and dried over anhydrous Na₂SO₄. After filtration, the filtrate was concentrated under reduced pressure to afford 3-methyl-1-{[4-({5H,6H,7H,8H-pyrido[3,4-d]pyrimidin-2-yl}amino)phenyl]methyl}azetidin-3-ol (150 mg; Crude Product).

tert-butyl 7-[2-({4-[(3-hydroxy-3-methylazetidin-1-yl)methyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate: To a stirred mixture of 3-methyl-1-{[4-({5H,6H,7H,8H-pyrido[3,4-d]pyrimidin-2-yl}amino)phenyl]methyl}azetidin-3-ol (140.00 mg; 0.39 mmol) and tert-butyl 7-bromo-8-methyl-1H,2H,3H-pyrido[2,3-b][1,4]oxazine-1-carboxylate (155.98 mg; 0.47 mmol) in 1,4-dioxane (15 mL) were added Pd-PEPPSI-IPentCl 2-methylpyridine (o-picoline) (34.92 mg; 0.04 mmol) and Cs₂CO₃ (406.28 mg; 1.18 mmol) at room temperature. After stirring for 4 h at 100 degrees C. under argon atmosphere, the mixture was concentrated under reduced pressure. The residue was purified by silica gel column chromatography and eluted with CH₂Cl₂/MeOH (9:1) to afford tert-butyl 7-[2-({4-[(3-hydroxy-3-methylazetidin-1-yl)methyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate as yellow solid (140 mg; 53.22%).

3-methyl-1-({4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}methyl)azetidin-3-ol: To a stirred solution of tert-butyl 7-[2-({4-[(3-hydroxy-3-methylazetidin-1-yl)methyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate (120 mg; 0.18 mmol) in DCM (5 mL) was added TFA (1 mL) at room temperature. The resulting mixture was stirred for 1 h at room temperature. The mixture was concentrated under reduced pressure. The crude product was purified by Prep-HPLC (Column: XBridge Prep OBD C18 Column, 30*150 mm, 5 μm; Mobile Phase A: water (10 mmol/L NH₄HCO₃+0.05% NH₄OH), Mobile Phase B: ACN; Flow rate: 60 mL/min.; Gradient: 22% B to 44% B in 8 min., 44% B; Wave Length: 254 nm; RTT (min.): 7; Number Of Runs: 2) to afford 3-methyl-1-({4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}methyl)azetidin-3-ol as yellow solid (16.10 mg; 17.19%).

HPLC 91.09% purity, 7.62 min., MS: m/s=474.20 [M+H]⁺. ¹H NMR (300 MHz, DMSO-d6): 9.46 (s, 1H), 8.32 (s, 1H), 7.72-7.60 (m, 2H), 7.29 (s, 1H), 7.17-7.09 (m, 2H), 5.57-5.49 (m, 1H), 5.11 (s, 1H), 4.24-4.15 (m, 2H), 3.95 (s, 2H), 3.48 (s, 2H), 3.31 (s, 2H), 3.17-3.07 (m, 4H), 2.89-2.74 (m, 4H), 2.04 (s, 3H), 1.34 (s, 3H).

### Example 124: Synthesis of Compound 124 N1-(2-(dimethylamino)ethyl)-2-fluoro-N4-(6-(8-methyl-2,3-dihydro-1H-pyrido[2,3-b][1,4]oxazin-7-yl)-5,6,7,8-tetrahydro-2,6-naphthyridin-3-yl)benzene-1,4-diamine

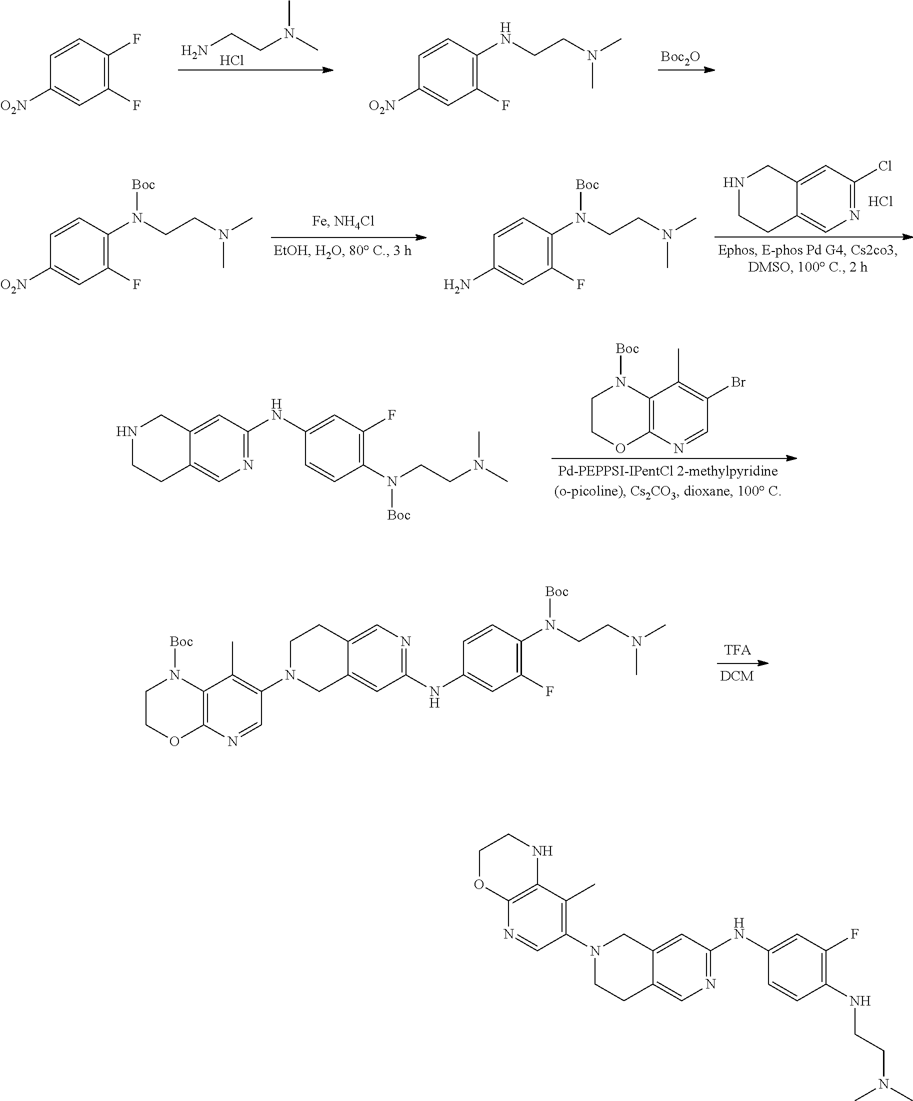

N-[2-(dimethylamino)ethyl]-2-fluoro-4-nitroaniline: A solution of 1,2-difluoro-4-nitrobenzene (3 g; 17.91 mmol) and (2-aminoethyl)dimethylamine (1.70 g; 18.32 mmol) and TEA (5.49 mL; 37.55 mmol) in AcOEt (100 mL) was stirred for overnight at 80 degrees C. under nitrogen atmosphere. The resulting mixture was concentrated under vacuum. The residue was purified by silica gel column chromatography and eluted with (PE:EtOAc=7:3) to afford N-[2-(dimethylamino)ethyl]-2-fluoro-4-nitroaniline (3.40 g; 83.5%).

tert-butyl N-[2-(dimethylamino)ethyl]-N-(2-fluoro-4-nitrophenyl)carbamate: A solution of N-[2-(dimethylamino)ethyl]-2-fluoro-4-nitroaniline (3.50 g; 15.12 mmol) and (Boc)₂O (17.25 mL; 76.61 mmol) and DMAP (200 mg; 1.47 mmol) and TEA (6.30 mL; 43.06 mmol) in DCM (50 mL) was stirred for overnight at room temperature under nitrogen atmosphere. The resulting mixture was concentrated under vacuum. The residue was purified by silica gel column chromatography and eluted with (PE:EtOAc=7:3) to afford tert-butyl N-[2-(dimethylamino)ethyl]-N-(2-fluoro-4-nitrophenyl)carbamate (4.80 g; 97.0%).

tert-butyl N-(4-amino-2-fluorophenyl)-N-[2-(dimethylamino)ethyl]carbamate: A solution of tert-butyl N-[2-(dimethylamino)ethyl]-N-(2-fluoro-4-nitrophenyl)carbamate (4.90 g; 14.97 mmol) and NH₄Cl (4 g; 74.03 mmol) and Fe (4 g; 70.19 mmol) in water (32 mL) and EtOH (48 mL) was stirred for 3 h at 80 degrees C. under nitrogen atmosphere. The resulting mixture was concentrated under vacuum. The residue was purified by silica gel column chromatography and eluted with (PE:EtOAc=1:99) to afford tert-butyl N-(4-amino-2-fluorophenyl)-N-[2-(dimethylamino)ethyl]carbamate as off-white solid (3.70 g; 83.1%).

tert-butyl N-[2-(dimethylamino)ethyl]-N-{2-fluoro-4-[(5,6,7,8-tetrahydro-2,6-naphthyridin-3-yl)amino]phenyl}carbamate: To a stirred mixture of tert-butyl N-(4-amino-2-fluorophenyl)-N-[2-(dimethylamino)ethyl]carbamate (200 mg; 0.65 mmol) and 7-chloro-1,2,3,4-tetrahydro-2,6-naphthyridine (119.89 mg; 0.71 mmol) in DMSO (15 mL) were added, and Cs₂CO₃ (638.15 mg; 1.94 mmol) in portions at 25 degrees C. The resulting mixture was stirred for 2 h at 100 degrees C. under nitrogen atmosphere. The reaction was quenched with H₂O at room temperature. The resulting mixture was extracted with EA (3×50 mL). The combined organic layers were washed with brine and dried over anhydrous MgSO₄. After filtration, the filtrate was concentrated under reduced pressure. The residue was purified by silica gel column chromatography and eluted with MeOH:DCM=1:10 to afford tert-butyl N-[2-(dimethylamino)ethyl]-N-{2-fluoro-4-[(5,6,7,8-tetrahydro-2,6-naphthyridin-3-yl)amino]phenyl}carbamate as yellow solid (250 mg; 59.9%).

tert-butyl 7-{7-[(4-{[(tert-butoxy)carbonyl][2-(dimethylamino)ethyl]amino}-3-fluorophenyl)amino]-1,2,3,4-tetrahydro-2,6-naphthyridin-2-yl}-8-methyl-1H,2H,3H-pyrido[2,3-b][1,4]oxazine-1-carboxylate: To a stirred mixture of tert-butyl N-[2-(dimethylamino)ethyl]-N-{2-fluoro-4-[(5,6,7,8-tetrahydro-2,6-naphthyridin-3-yl)amino]phenyl}carbamate (230 mg; 0.36 mmol) and tert-butyl 7-bromo-8-methyl-1H,2H,3H-pyrido[2,3-b][1,4]oxazine-1-carboxylate (141 mg; 0.43 mmol) in 1,4-dioxane (15 mL) were added Pd-PEPPSI-IPentCl 2-methylpyridine (o-picoline) (32 mg; 0.04 mmol) and Cs₂CO₃ (352 mg; 1.07 mmol) in portions at 25 degrees C. The resulting mixture was stirred for 2 h at 100 degrees C. under nitrogen atmosphere. The resulting mixture was concentrated under vacuum. The residue was purified by silica gel column chromatography and eluted with MeOH:DCM=12:88 to afford tert-butyl 7-{7-[(4-{[(tert-butoxy)carbonyl][2-(dimethylamino)ethyl]amino}-3-fluorophenyl)amino]-1,2,3,4-tetrahydro-2,6-naphthyridin-2-yl}-8-methyl-1H,2H,3H-pyrido[2,3-b][1,4]oxazine-1-carboxylate as yellow solid (150 mg; 36.3%).

N1-[2-(dimethylamino)ethyl]-2-fluoro-N4-(6-{8-methyl-1H,2H,3H-pyrido[2,3-b][1,4]oxazin-7-yl}-5,6,7,8-tetrahydro-2,6-naphthyridin-3-yl)benzene-1,4-diamine: To a stirred mixture of tert-butyl 7-{7-[(4-{[(tert-butoxy)carbonyl][2-(dimethylamino)ethyl]amino}-3-fluorophenyl)amino]-1,2,3,4-tetrahydro-2,6-naphthyridin-2-yl}-8-methyl-1H,2H,3H-pyrido[2,3-b][1,4]oxazine-1-carboxylate (130 mg; 0.11 mmol) in DCM (8 mL) was added TFA (2 mL) in portions at 25 degrees C. The resulting mixture was stirred for 3 h at 25 degrees C. under nitrogen atmosphere. The resulting mixture was concentrated under vacuum. The crude product was purified by Prep-HPLC with the following conditions (Column: Atlantis Prep T3 OBD Column, 19*250 mm 10 μm; Flow rate: 20 mL/min; Gradient: 3% B to 22% B in 10 min, Wave Length: 254 nm; RT1 (min.): 8.5; Number Of Runs: 2) to afford N1-[2-(dimethylamino)ethyl]-2-fluoro-N4-(6-{8-methyl-1H,2H,3H-pyrido[2,3-b][1,4]oxazin-7-yl}-5,6,7,8-tetrahydro-2,6-naphthyridin-3-yl)benzene-1,4-diamine; formic acid as light yellow solid (18.90 mg; 32.2%).

HPLC 99.93% purity, 2.49 min., MS: m/s=478.30[M+H]⁺. ¹H NMR (400 MHz, DMSO-d6): 8.55 (s, 1H), 7.93 (s, 1H), 7.51 (dd, J=14.4, 2.4 Hz, 1H), 7.25 (s, 1H), 7.06 (d, J=8.7 Hz, 1H), 6.67 (t, J=9.4 Hz, 1H), 6.46 (s, 1H), 5.50 (s, 1H), 4.65 (s, 1H), 4.18 (t, J=4.4 Hz, 2H), 3.95 (s, 2H), 3.29 (s, 4H), 3.09 (dd, J=11.6, 5.8 Hz, 4H), 2.79 (t, J=5.8 Hz, 2H), 2.47 (d, J=6.4 Hz, 2H), 2.19 (s, 5H), 2.02 (s, 2H).

### Example 125: Synthesis of 7-{8-methyl-1H,2H,3H-pyrido[2,3-b][1,4]oxazin-7-yl}-N-{4-[(pyrrolidin-1-yl)methyl]phenyl}-5H,6H,7H,8H-pyrido[3,4-d]pyrimidin-2-amine

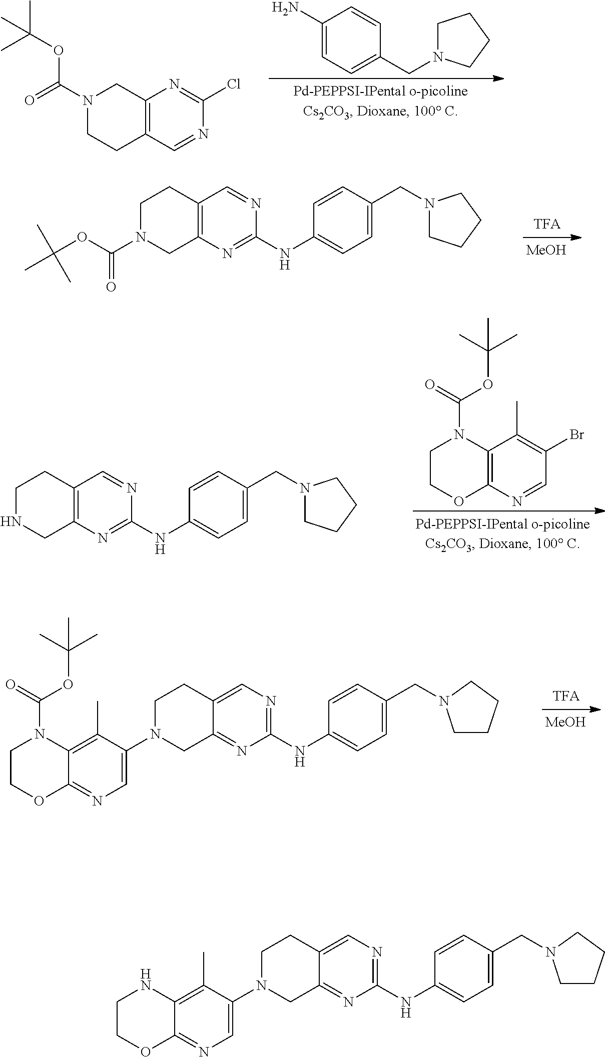

Tert-butyl 2-({4-[(pyrrolidin-1-yl)methyl]phenyl}amino)-5H,6H,7H,8H-pyrido[3,4-d]pyrimidine-7-carboxylate: To a suspension of tert-butyl 2-chloro-5H,6H,7H,8H-pyrido[3,4-d]pyrimidine-7-carboxylate (1.00 g; 3.71 mmol; 1.00 eq.) in 1,4-dioxane (40 mL) was added 4-[(pyrrolidin-1-yl)methyl]aniline (0.98 g; 5.56 mmol; 1.50 eq.), Cs₂CO₃ (3.62 g; 11.12 mmol; 3.00 eq.). The brown mixture was degassed with argon for 5 minutes, then Pd-PEPPSI-iPent-o-picoline (360.65 mg; 0.37 mmol; 0.10 eq.) was added and capped. The mixture was stirred at 100° C. for 16 hours. LCMS showed the reaction was complete and desired product was observed. The insoluble was removed by filtration. The solvent was concentrated to obtain tert-butyl 2-({4-[(pyrrolidin-1-yl)methyl]phenyl}amino)-5H,6H,7H,8H-pyrido[3,4-d]pyrimidine-7-carboxylate (500.00 mg; 39.2% yield) as pale yellow powder, used in the next step without further purification. MS: m/z 410 (M+H).

N-{4-[(pyrrolidin-1-yl)methyl]phenyl}-5H,6H,7H,8H-pyrido[3,4-d]pyrimidin-2-amine: To a vial was added tert-butyl 2-({4-[(pyrrolidin-1-yl)methyl]phenyl}amino)-5H,6H,7H,8H-pyrido[3,4-d]pyrimidine-7-carboxylate (500.00 mg; 1.22 mmol; 1.00 eq.) in MeOH (2 mL) was added trifluoroacetic acid (6960.69 mg; 61.05 mmol; 50.00 eq.). The mixture was stirred at room temperature for 1 hour. The LC/MS showed the reaction was complete and the desired product (m/z: 310) was observed. The solvent was removed and the residue was dissolved in DMSO (10 mL) and was purified directly on InterChim prep-HPLC: 20-95% MeCN in water (0.1% ammonium hydroxide) 10 min. run with a flow rate of 60 mL/min. to provide the desired product N-{4-[(pyrrolidin-1-yl)methyl]phenyl}-5H,6H,7H,8H-pyrido[3,4-d]pyrimidin-2-amine (300.00 mg; 79.4% yield). MS: m/z 310 (M+H).

Tert-butyl 8-methyl-7-[2-({4-[(pyrrolidin-1-yl)methyl]phenyl}amino)-5H,6H,7H,8H-pyrido[3,4-d]pyrimidin-7-yl]-1H,2H,3H-pyrido[2,3-b][1,4]oxazine-1-carboxylate: To a stirred solution of N-{4-[(pyrrolidin-1-yl)methyl]phenyl}-5H,6H,7H,8H-pyrido[3,4-d]pyrimidin-2-amine (240.00 mg; 0.70 mmol; 1.00 eq.) and tert-butyl 7-bromo-8-methyl-1H,2H,3H-pyrido[2,3-b][1,4]oxazine-1-carboxylate (241.68 mg; 0.70 mmol; 1.00 eq.) in was added Pd-PEPPSI-IPent Cl 2-methylpyridine (o-picoline) (185.21 mg; 0.21 mmol; 0.30 eq.) and cesium carbonate (478.86 mg; 1.40 mmol; 2.00 eq.) in 1,4-dioxane (15 mL). The resulting mixture was stirred for 4 h at 100 degrees C. under nitrogen atmosphere. The resulting mixture was concentrated under reduced pressure. The residue was purified by silica gel column chromatography and eluted with CH₂Cl₂/MeOH (5:1) to afford tert-butyl 8-methyl-7-[2-({4-[(pyrrolidin-1-yl)methyl]phenyl}amino)-5H,6H,7H,8H-pyrido[3,4-d]pyrimidin-7-yl]-1H,2H,3H-pyrido[2,3-b][1,4]oxazine-1-carboxylate (170.00 mg; 43% yield). MS: m/z (558, M+H).

7-{8-methyl-1H,2H,3H-pyrido[2,3-b][1,4]oxazin-7-yl}-N-{4-[(pyrrolidin-1-yl)methyl]phenyl}-5H,6H,7H,8H-pyrido[3,4-d]pyrimidin-2-amine: To a suspension of tert-butyl 8-methyl-7-[2-({4-[(pyrrolidin-1-yl)methyl]phenyl}amino)-5H,6H,7H,8H-pyrido[3,4-d]pyrimidin-7-yl]-1H,2H,3H-pyrido[2,3-b][1,4]oxazine-1-carboxylate (100.00 mg; 0.18 mmol; 1.00 eq.) in MeOH (1.5 mL) was added trifluoroacetic acid (2044.57 mg; 17.93 mmol; 100.00 eq.). The mixture was stirred for 20 minutes. After the solvent was removed, the residue was dissolved in DMF/water (10/5 mL) and neutralized with addition of Na₂CO₃. The solid was removed with filtration. The mixture was purified through InterChim reverse phase with acetonitrile in 0.1 NH₄OH from 0% to 100% in 14 minutes with a flow rate of 30 mL/min. to provide the desired product 7-{8-methyl-1H,2H,3H-pyrido[2,3-b][1,4]oxazin-7-yl}-N-{4-[(pyrrolidin-1-yl)methyl]phenyl}-5H,6H,7H,8H-pyrido[3,4-d]pyrimidin-2-amine (49.50 mg; 60.3% yield).

m/z: 458 (M+H). ¹H NMR (400 MHz, DMSO) δ 9.46 (s, 1H), 8.32 (s, 1H), 7.68 (d, J=8.3 Hz, 2H), 7.29 (s, 1H), 7.18 (d, J=8.2 Hz, 2H), 5.51 (s, 1H), 4.19 (t, J=4.4 Hz, 2H), 3.95 (s, 2H), 3.53 (s, 2H), 3.12 (t, J=5.7 Hz, 2H), 2.79 (t, J=5.6 Hz, 2H), 2.45 (s, 6H), 2.04 (s, 3H), 1.70 (s, 4H).

### Example 126: Synthesis of 7-{8-methyl-1H,2H,3H-pyrido[2,3-b][1,4]oxazin-7-yl}-N-{4-[(1H-1,2,4-triazol-1-yl)methyl]phenyl}-5H,6H,7H,8H-pyrido[3,4-d]pyrimidin-2-amine

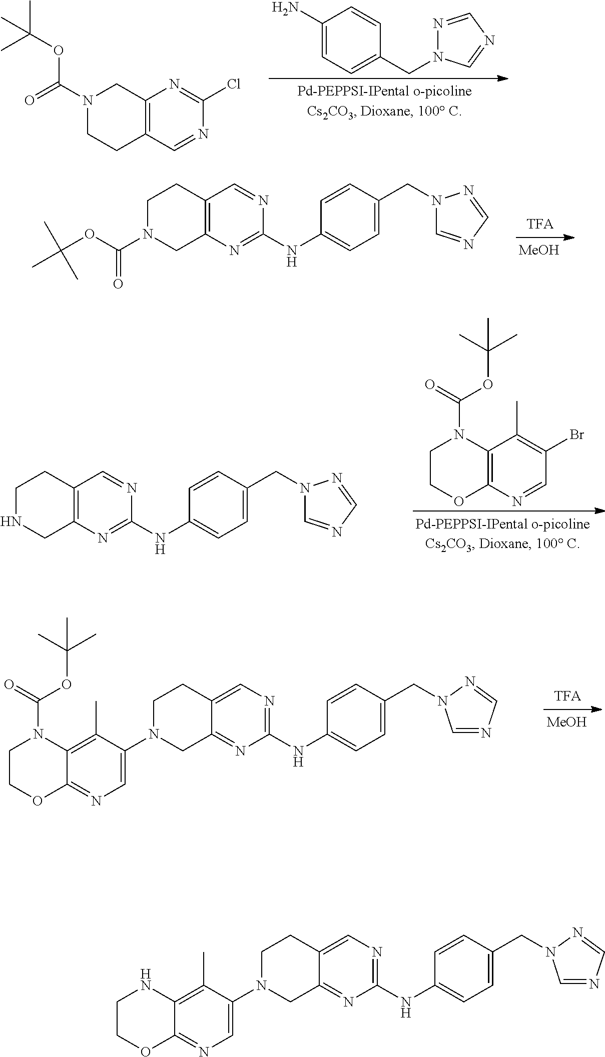

Tert-butyl 2-({4-[(1H-1,2,4-triazol-1-yl)methyl]phenyl}amino)-5H,6H,7H,8H-pyrido[3,4-d]pyrimidine-7-carboxylate: In a 40 mL vial were added tert-butyl 2-chloro-5H,6H,7H,8H-pyrido[3,4-d]pyrimidine-7-carboxylate (0.70 g; 2.60 mmol; 1.00 eq.), 4-[(1H-1,2,4-triazol-1-yl)methyl]aniline (0.54 g; 3.11 mmol; 1.20 eq.), CESIUM CARBONATE (2.54 g; 7.79 mmol; 3.00 eq.) in dioxane (20 mL) to give a suspension. The brown mixture was degassed with argon for 5 minutes, then Pd-PEPPSI-iPent-o-picoline (252.46 mg; 0.26 mmol; 0.10 eq.) was added and capped. The mixture was heated at 100° C. for 16 hours. LCMS showed the reaction was complete and desired product was observed. The insoluble was removed by filtration. The solvent was concentrated to get tert-butyl 2-({4-[(1H-1,2,4-triazol-1-yl)methyl]phenyl}amino)-5H,6H,7H,8H-pyrido[3,4-d]pyrimidine-7-carboxylate (1058.00 mg; 100%) as pale yellow powder, used in the next step. MS: m/z 408 (M+H).

N-{4-[(1H-1,2,4-triazol-1-yl)methyl]phenyl}-5H,6H,7H,8H-pyrido[3,4-d]pyrimidin-2-amine: To a suspension of tert-butyl 2-({4-[(1H-1,2,4-triazol-1-yl)methyl]phenyl}amino)-5H,6H,7H,8H-pyrido[3,4-d]pyrimidine-7-carboxylate (1000.00 mg; 2.45 mmol; 1.00 eq.) in MeOH (3 mL) was added trifluoroacetic acid (27983.23 mg; 245.42 mmol; 100.00 eq.). The mixture was stirred for 20 minutes. After the solvent was removed, the residue was purified through InterChim reverse phase with acetonitrile in 0.1 NH₄OH from 0% to 100% in 14 minutes with a flow rate of 30 mL/min. to provide the desired product N-{4-[(1H-1,2,4-triazol-1-yl)methyl]phenyl}-5H,6H,7H,8H-pyrido[3,4-d]pyrimidin-2-amine (500.00 mg; 66.3% yield). MS: m/z 308 (M+H).

Tert-butyl 8-methyl-7-[2-({4-[(1H-1,2,4-triazol-1-yl)methyl]phenyl}amino)-5H,6H,7H,8H-pyrido[3,4-d]pyrimidin-7-yl]-1H,2H,3H-pyrido[2,3-b][1,4]oxazine-1-carboxylate: To a stirred solution of N-{4-[(1H-1,2,4-triazol-1-yl)methyl]phenyl}-5H,6H,7H,8H-pyrido[3,4-d]pyrimidin-2-amine (270.00 mg; 0.79 mmol; 1.00 eq.), tert-butyl 7-bromo-8-methyl-1H,2H,3H-pyrido[2,3-b][1,4]oxazine-1-carboxylate (273.70 mg; 0.79 mmol; 1.00 eq.) and cesium carbonate (542.32 mg; 1.58 mmol; 2.00 eq.) in 1,4-dioxane (15 mL) was added Pd-PEPPSI-IPent Cl 2-methylpyridine (o-picoline) (209.76 mg; 0.24 mmol; 0.30 eq). The resulting mixture was sealed and stirred for overnight at 100 degrees C. under nitrogen atmosphere. After filtration and removal of the solvent, the residue was dissolved in DMF. The mixture was purified through InterChim reverse phase with acetonitrile in water containing 0.1% formic acid from 0% to 100% in 14 minutes with a flow rate of 26 mL/min. to provide the desired product tert-butyl 8-methyl-7-[2-({4-[(1H-1,2,4-triazol-1-yl)methyl]phenyl}amino)-5H,6H,7H,8H-pyrido[3,4-d]pyrimidin-7-yl]-1H,2H,3H-pyrido[2,3-b][1,4]oxazine-1-carboxylate (230.00 mg; 52.4%). MS: m/z 556 (M+H).

7-{8-methyl-1H,2H,3H-pyrido[2,3-b][1,4]oxazin-7-yl}-N-{4-[(1H-1,2,4-triazol-1-yl)methyl]phenyl}-5H,6H,7H,8H-pyrido[3,4-d]pyrimidin-2-amine: In a flask were added tert-butyl 8-methyl-7-[2-({4-[(1H-1,2,4-triazol-1-yl)methyl]phenyl}amino)-5H,6H,7H,8H-pyrido[3,4-d]pyrimidin-7-yl]-1H,2H,3H-pyrido[2,3-b][1,4]oxazine-1-carboxylate (100.00 mg; 0.18 mmol; 1.00 eq.) in MeOH (1.5 mL) and trifluoroacetic acid (2052.13 mg; 18.00 mmol; 100.00 eq.). The mixture was stirred at for 20 minutes. After the solvent was removed, the residue was dissolved in DMF/water (20/5 mL) and neutralized with addition of Na₂CO₃. The solid was removed with filtration. The mixture was purified through InterChim reverse phase with acetonitrile in 0.1 NH₄OH from 0% to 100% in 14 minutes with a flow rate of 30 mL/min. to provide the desired product 7-{8-methyl-1H,2H,3H-pyrido[2,3-b][1,4]oxazin-7-yl}-N-{4-[(1H-1,2,4-triazol-1-yl)methyl]phenyl}-5H,6H,7H,8H-pyrido[3,4-d]pyrimidin-2-amine (31.50 mg; 33% yield). MS: m/z 456 (M+H). ¹H NMR (500 MHz, DMSO) δ 9.58 (s, 1H), 8.61 (d, J=2.8 Hz, 1H), 8.33 (s, 1H), 7.96 (d, J=2.8 Hz, 1H), 7.72 (d, J=8.1 Hz, 2H), 7.24 (dd, J=36.0, 5.6 Hz, 3H), 5.53 (s, 1H), 5.31 (s, 2H), 4.19 (d, J=5.1 Hz, 2H), 3.95 (s, 2H), 3.20 (d, J=92.9 Hz, 4H), 2.78 (d, J=7.4 Hz, 2H), 2.03 (d, J=2.8 Hz, 3H).

### Example 127: Synthesis of N-{4-[(1H-imidazol-1-yl) methyl]phenyl}-7-{8-methyl-1H,2H,3H-pyrido[2,3-b][1,4]oxazin-7-yl}-5H,6H,7H,8H-pyrido[3,4-d]pyrimidin-2-amine

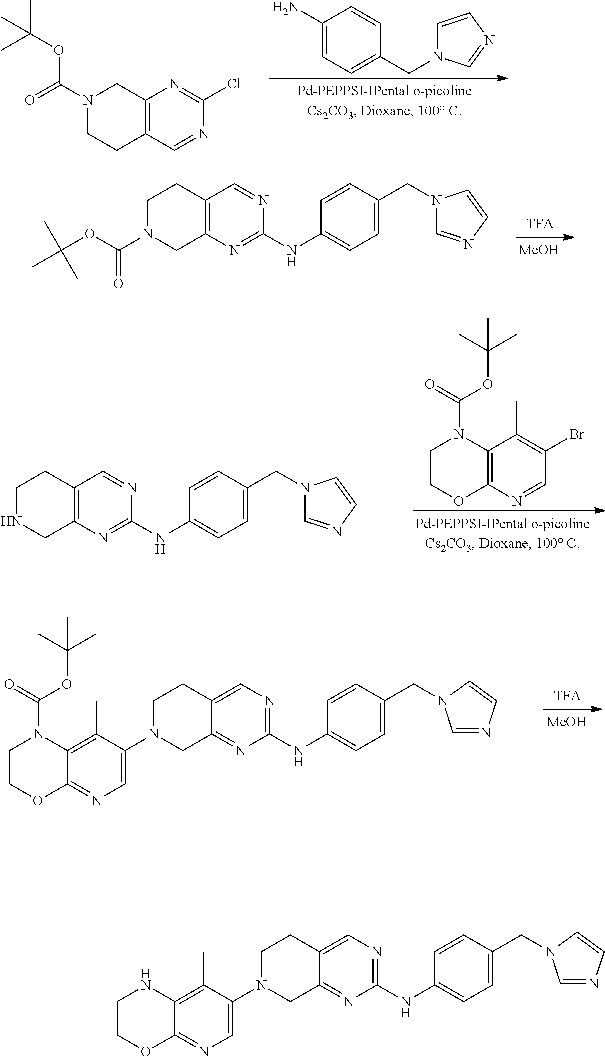

Tert-butyl 2-({4-[(1H-imidazol-1-yl)methyl]phenyl}amino)-5H,6H,7H,8H-pyrido[3,4-d]pyrimidine-7-carboxylate: In a 40 mL vial were added tert-butyl 2-chloro-5H,6H,7H,8H-pyrido[3,4-d]pyrimidine-7-carboxylate (0.70 g; 2.60 mmol; 1.00 eq.), 4-[(1H-imidazol-1-yl)methyl]aniline (0.54 g; 3.11 mmol; 1.20 eq.), CESIUM CARBONATE (2.54 g; 7.79 mmol; 3.00 eq.) in dioxane (20 mL). The brown suspended mixture was degassed with Argon for 5 minutes, then Pd-PEPPSI-IPent Cl 2-methylpyridine (o-picoline) (252.46 mg; 0.26 mmol; 0.10 eq.) was added and capped. The mixture was heated at 100° C. for 16 hours. LCMS showed the reaction was complete and desired product was observed. The insoluble was removed by filtration. The solvent was concentrated and to get tert-butyl 2-({4-[(1H-imidazol-1-yl)methyl]phenyl}amino)-5H,6H,7H,8H-pyrido[3,4-d]pyrimidine-7-carboxylate (1000.00 mg; 94.8% yield), used in the next step. MS: m/z 407 (M+H).

N-{4-[(1H-imidazol-1-yl)methyl]phenyl}-5H,6H,7H,8H-pyrido[3,4-d]pyrimidin-2-amine: In a 40 mL vial were added tert-butyl 2-({4-[(1H-1,2,4-triazol-1-yl)methyl]phenyl}amino)-5H,6H,7H,8H-pyrido[3,4-d]pyrimidine-7-carboxylate (1000.00 mg; 2.45 mmol; 1.00 eq.) in MeOH (3 mL) was added trifluoroacetic acid (27983.23 mg; 245.42 mmol; 100.00 eq.). The mixture was stirred at for 20 minutes. After the solvent was removed, the residue was purified through InterChim reverse phase with acetonitrile in 0.1 NH₄OH from 0% to 100% in 14 minutes with a flow rate of 30 mL/min. to provide the desired product N-{4-[(1H-imidazol-1-yl)methyl]phenyl}-5H,6H,7H,8H-pyrido[3,4-d]pyrimidin-2-amine (650.00 mg; 86.5% yield). MS: m/z 307 (M+H).

Tert-butyl 7-[2-({4-[(1H-imidazol-1-yl)methyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate: To a stirred solution of N-{4-[(1H-1,2,4-triazol-1-yl)methyl]phenyl}-5H,6H,7H,8H-pyrido[3,4-d]pyrimidin-2-amine (400.00 mg; 1.17 mmol; 1.00 eq.) and tert-butyl 7-bromo-8-methyl-1H,2H,3H-pyrido[2,3-b][1,4]oxazine-1-carboxylate (405.49 mg; 1.17 mmol; 1.00 eq.) and cesium carbonate (803.43 mg; 2.34 mmol; 2.00 eq.) in 1,4-dioxane (15 mL) was added Pd-PEPPSI-IPent Cl 2-methylpyridine (o-picoline) (310.76 mg; 0.35 mmol; 0.30 eq.). The resulting mixture was stirred for 4 hours at 100° C. under nitrogen atmosphere. After filtration and removal of the solvent, the residue was dissolved in DMF. The mixture was purified through InterChim reverse phase with acetonitrile in water containing 0.1% FA from 0% to 100% in 14 minutes with a flow rate of 26 mL/min. to provide the desired product tert-butyl 7-[2-({4-[(1H-imidazol-1-yl)methyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate (250.00 mg; 38.5% yield). MS: m/z 555 (M+H).

N-{4-[(1H-imidazol-1-yl)methyl]phenyl}-7-{8-methyl-1H,2H,3H-pyrido[2,3-b][1,4]oxazin-7-yl}-5H,6H,7H,8H-pyrido[3,4-d]pyrimidin-2-amine: In a flask were added tert-butyl 7-[2-({4-[(1H-imidazol-1-yl)methyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate (100.00 mg; 0.18 mmol; 1.00 eq.) in MeOH (1.5 mL) and trifluoroacetic acid (2055.79 mg; 18.03 mmol; 100.00 eq.). The mixture was stirred at for 20 minutes. After the solvent was removed, the residue was dissolved in DMF/water (20/5 mL) and neutralized with addition of Na₂CO₃. The solid was removed with filtration. The mixture was purified through InterChim reverse phase with acetonitrile in 0.1 NH₄OH from 0% to 100% in 14 minutes with a flow rate of 30 mL/min. to provide the desired product N-{4-[(1H-imidazol-1-yl)methyl]phenyl}-7-{8-methyl-1H,2H,3H-pyrido[2,3-b][1,4]oxazin-7-yl}-5H,6H,7H,8H-pyrido[3,4-d]pyrimidin-2-amine (37.30 mg, 39.6% yield).

MS: m/z 455 (M+H). ¹H NMR (500 MHz, DMSO) δ 9.57 (s, 1H), 8.33 (s, 1H), 7.79-7.64 (m, 3H), 7.28 (s, 1H), 7.24-7.13 (m, 2H), 6.88 (s, 1H), 5.54 (q, J=4.6, 2.8 Hz, 1H), 5.09 (s, 2H), 4.26-3.87 (m, 4H), 3.33-3.21 (m, 3H), 3.11 (t, J=5.8 Hz, 2H), 2.78 (t, J=5.6 Hz, 2H), 2.03 (s, 3H).

### Example 128: Synthesis of {4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}methanesulfonamide

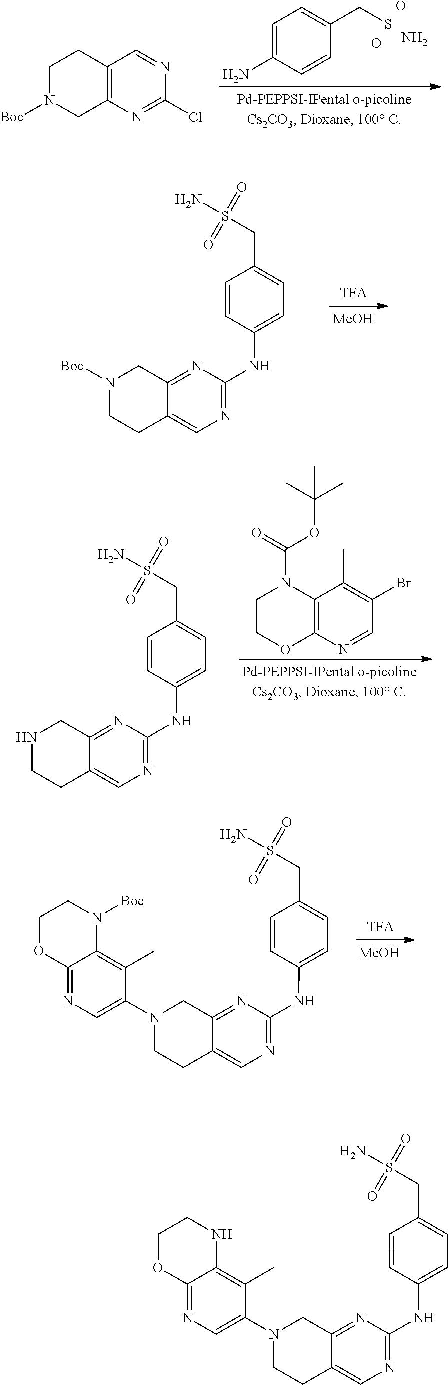

tert-butyl 2-{[4-(sulfamoylmethyl)phenyl]amino}-5H,6H,7H,8H-pyrido[3,4-d]pyrimidine-7-carboxylate: In a 40 mL vial were added tert-butyl 2-chloro-5H,6H,7H,8H-pyrido[3,4-d]pyrimidine-7-carboxylate (0.70 g; 2.60 mmol; 1.00 eq.), (4-aminophenyl)methanesulfonamide (0.58 g; 3.11 mmol; 1.20 eq.), CESIUM CARBONATE (2.54 g; 7.79 mmol; 3.00 eq.) in dioxane (20 mL). The brown mixture was degassed with argon for 5 minutes, then Pd-PEPPSI-IPentCl 2-methylpyridine (o-picoline) (252.46 mg; 0.26 mmol; 0.10 eq.) was added and capped. The mixture was heated at 100° C. for 16 h. LCMS showed the reaction was complete and desired product was observed. The insoluble was removed by filtration. The solvent was concentrated and to get tert-butyl 2-{[4-(sulfamoylmethyl)phenyl]amino}-5H,6H,7H,8H-pyrido[3,4-d]pyrimidine-7-carboxylate (1000.00 mg; 91.9% yield) as pale yellow powder, used in the next step. MS: m/z 420 (M+H).

[4-({5H,6H,7H,8H-pyrido[3,4-d]pyrimidin-2-yl}amino)phenyl]methanesulfonamide: In a flask were added tert-butyl 2-{[4-(sulfamoylmethyl)phenyl]amino}-5H,6H,7H,8H-pyrido[3,4-d]pyrimidine-7-carboxylate (1000.00 mg; 2.38 mmol; 1.00 eq.) in MeOH (3 mL) and trifluoroacetic acid (27180.76 mg; 238.38 mmol; 100.00 eq.). The mixture was stirred at for 20 minutes. After the solvent was removed, the residue was purified through InterChim reverse phase with acetonitrile in 0.1 NH₄OH from 0% to 100% in 14 minutes with a flow rate of 30 mL/min. to provide the desired product [4-({5H,6H,7H,8H-pyrido[3,4-d]pyrimidin-2-yl}amino)phenyl]methanesulfonamide (700.00 mg; 91.9% yield). MS: m/z 320 (M+H).

tert-butyl 8-methyl-7-(2-{[4-(sulfamoylmethyl)phenyl]amino}-5H,6H,7H,8H-pyrido[3,4-d]pyrimidin-7-yl)-1H,2H,3H-pyrido[2,3-b][1,4]oxazine-1-carboxylate: To a stirred solution of [4-({5H,6H,7H,8H-pyrido[3,4-d]pyrimidin-2-yl}amino)phenyl]methanesulfonamide (320.00 mg; 0.90 mmol; 1.00 eq.), tert-butyl 7-bromo-8-methyl-1H,2H,3H-pyrido[2,3-b][1,4]oxazine-1-carboxylate (312.17 mg; 0.90 mmol; 1.00 eq.) and cesium carbonate (618.54 mg; 1.80 mmol; 2.00 eq.) in 1,4-dioxane (15 mL) in was added Pd-PEPPSI-IPentCl 2-methylpyridine (o-picoline) (239.24 mg; 0.27 mmol; 0.30 eq.). The resulting mixture was stirred for 4 h at 100 degrees C. under nitrogen atmosphere. After filtration and removal of the solvent, the residue was dissolved in DMF. The mixture was purified through InterChim reverse phase with acetonitrile in water containing 0.1% FA from 0% to 100% in 14 minutes with a flow rate of 26 mL/min. to provide the desired product tert-butyl 8-methyl-7-(2-{[4-(sulfamoylmethyl)phenyl]amino}-5H,6H,7H,8H-pyrido[3,4-d]pyrimidin-7-yl)-1H,2H,3H-pyrido[2,3-b][1,4]oxazine-1-carboxylate (40.00 mg; 7.8% yield). MS: m/z 568 (M+H).

{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}methanesulfonamide: In a flask were added tert-butyl 8-methyl-7-(2-{[4-(sulfamoylmethyl)phenyl]amino}-5H,6H,7H,8H-pyrido[3,4-d]pyrimidin-7-yl)-1H,2H,3H-pyrido[2,3-b][1,4]oxazine-1-carboxylate (20.00 mg; 0.04 mmol; 1.00 eq.) in MeOH (1.5 mL) and trifluoroacetic acid (401.73 mg; 3.52 mmol; 100.00 eq.). The mixture was stirred at for 20 minutes. After the solvent was removed, the residue was dissolved in DMF/water (20/5 mL) and neutralized with addition of Na₂CO₃. The solid was removed with filtration. The mixture was purified through InterChim reverse phase with acetonitrile in 0.1 NH₄OH from 0% to 100% in 14 minutes with a flow rate of 30 mL/min. to provide the desired product {4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}methanesulfonamide (3.00 mg; 18% yield).

MS: m/z 468 (M+H). ¹H NMR (400 MHz, DMSO) δ 9.57 (s, 1H), 8.35 (s, 1H), 7.75 (d, J=8.1 Hz, 2H), 7.29 (s, 1H), 7.24 (d, J=8.1 Hz, 2H), 6.75 (s, 2H), 5.52 (s, 1H), 4.19 (d, J=7.8 Hz, 4H), 3.97 (s, 2H), 3.13 (s, 2H), 2.80 (s, 2H), 2.05 (d, J=2.3 Hz, 3H), 1.24 (s, 2H).

### Example 129: 6-methyl-N-[7-(pyridin-2-yl)-5H,6H,7H,8H-pyrido[3,4-d]pyrimidin-2-yl]-5,6,7,8-tetrahydro-1,6-naphthyridin-3-amine

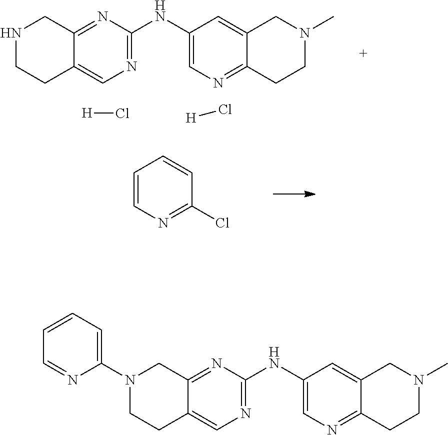

A solution of 6-methyl-N-{5H,6H,7H,8H-pyrido[3,4-d]pyrimidin-2-yl}-5,6,7,8-tetrahydro-1,6-naphthyridin-3-amine dihydrochloride (150.00 mg; 0.37 mmol; 1.00 eq.), 2-chloropyridine (132.60 mg; 1.12 mmol; 3.00 eq.) in IPrOH (0.50 mL) was added DIEA (192.82 mg; 1.49 mmol; 4.00 eq.). Closed contained. purged with nitrogen and closed. Then the mixture was stirred at 135° C. under N₂ atmosphere for 18 hours. The LCMS shown reactant retained, 5.2% desired. reactant 2 used excess. The crude was concentrated and directly purified by C18 biotage column 5-95% ACN-0.1% aqueous ammonium bicarbonate solution to afford desired compound as yellow solid 6-methyl-N-[7-(pyridin-2-yl)-5H,6H,7H,8H-pyrido[3,4-d]pyrimidin-2-yl]-5,6,7,8-tetrahydro-1,6-naphthyridin-3-amine (25.00 mg; 0.06 mmol; 16.8%; yellow solid).

¹H NMR (400 MHz, Methanol-d₄) δ 8.65 (d, J=2.4 Hz, 1H), 8.30 (s, 1H), 8.15 (dd, J=5.0, 1.1 Hz, 1H), 8.10 (d, J=2.2 Hz, 1H), 7.65-7.58 (m, 1H), 6.95 (d, J=8.6 Hz, 1H), 6.71 (dd, J=6.8, 5.3 Hz, 1H), 4.63 (s, 2H), 3.91 (t, J=5.7 Hz, 2H), 3.69 (s, 2H), 3.01 (t, J=6.0 Hz, 2H), 2.90-2.83 (m, 4H), 2.52 (s, 3H).

### Example 130: Synthesis of Compound 4-[(7-{8-chloro-1H,2H,3H-pyrido[2,3-b][1,4]oxazin-7-yl}-5H,6H,7H,8H-pyrido[3,4-d]pyrimidin-2-yl)amino]-N,2-dimethylbenzamide

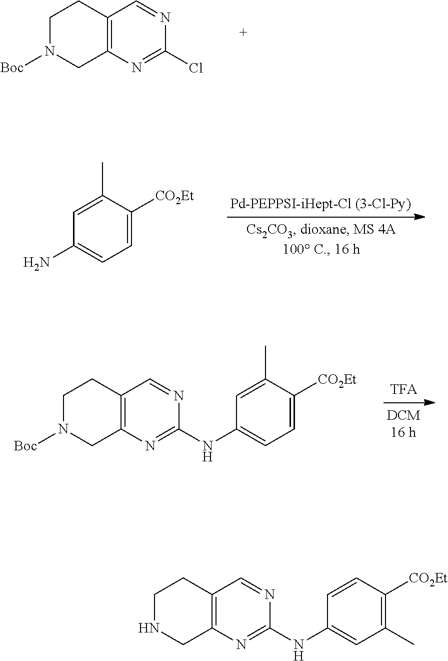 [SMILES: CC(C)(C)OC(=O)N1CCc2cnc(Cl)nc2C1 ; CCOC(=O)c1ccc(N)cc1C ; CCOC(=O)c1ccc(Nc2ncc3c(n2)CN(C(=O)OC(C)(C)C)CC3)cc1C ; CCOC(=O)c1ccc(Nc2ncc3c(n2)CNCC3)cc1C]

tert-butyl 2-{[4-(ethoxycarbonyl)-3-methylphenyl]amino}-5H,6H,7H,8H-pyrido[3,4-d]pyrimidine-7-carboxylate: 7-Boc-2-chloro-5,6,7,8-tetrahydropyrido[3,4-d]pyrimidine (0.390 g; 1.446 mmol; 1.0 eq.), ethyl 4-amino-2-methylbenzoate (0.285 g; 1.590 mmol; 1.1 eq.), cesium carbonate (1.413 g; 4.338 mmol; 3.0 eq.) and some MS 4A (200 mg) were added in dioxane (7 mL) to give a suspension, which was degassed with nitrogen. Pd-PEPPSI-iHept-Cl 3-ClPy (28.131 mg; 0.029 mmol; 0.020 eq.) was added in one portion. The mixture was degassed with nitrogen and heated at 100° C. for 16 h. It was cooled down to rt. DCM (10 mL) was added and stirred. The suspension was filtered through Celite and washed by DCM (3×6 mL). The combined DCM filtrates were concentrated and the residue was purified by silica gel column 0-80% EtOAc/Hexanes to give tert-butyl 2-{[4-(ethoxycarbonyl)-3-methylphenyl]amino}-5H,6H,7H,8H-pyrido[3,4-d]pyrimidine-7-carboxylate (0.596 g; 1.446 mmol; 99%) as pale yellow powder.

ethyl 2-methyl-4-({5H,6H,7H,8H-pyrido[3,4-d]pyrimidin-2-yl}amino)benzoate: DCM (6.0 mL) and TFA (6.0 mL; 80.773 mmol; 56 eq.) were added to tert-butyl 2-[(3-methanesulfonylphenyl)amino]-5H,6H,7H,8H-pyrido[3,4-d]pyrimidine-7-carboxylate (0.520 g; 1.286 mmol; 1.0 eq.) to give a red solution formed, which was stirred at room temperature for 16 h. Toluene (20 mL) was added and the mixture was concentrated. The residue was dried in vacuum oven to give ethyl 2-methyl-4-({5H,6H,7H,8H-pyrido[3,4-d]pyrimidin-2-yl}amino)benzoate (0.450 g; 1.445 mmol; 99%) as yellow powder.

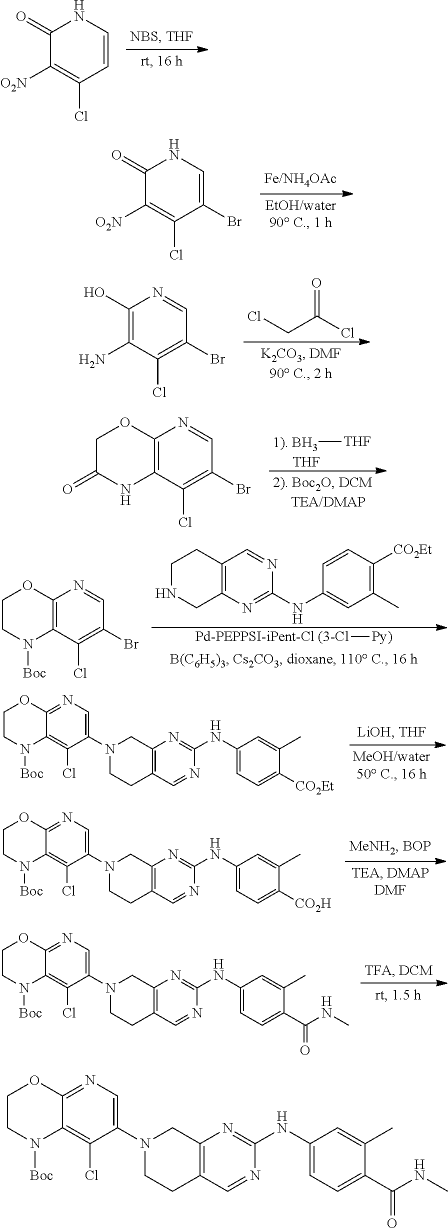

5-bromo-4-chloro-3-nitropyridin-2-ol: 4-Chloro-2-hydroxy-3-nitropyridine (12.0 g; 68.8 mmol; 1.0 eq.) and N-Bromosuccinimide (20.8 g; 116.9 mmol; 1.7 eq.) were mixed with THF (130 mL) to give a yellow suspension, it was stirred at room temperature for 16 h. The solution was concentrated, the residue was added 250 mL water, stirred, and heated at 45° C. for 30 min. The yellow suspension was sonicated, filtered, washed by water (3×50 mL) and pentane (2×25 mL). The collected solid was dried in vacuum oven for 16 h to give 5-bromo-4-chloro-3-nitropyridin-2-ol (16.7 g; 66.1 mmol; 96.1%) as pale yellow powder.

3-amino-5-bromo-4-chloropyridin-2-ol: 5-bromo-4-chloro-3-nitropyridin-2-ol (8.3 g; 32.7 mmol; 1.0 eq.), iron (9.1 g; 163.7 mmol; 5.0 eq.) and ammonium chloride (8.76 g; 163.7 mmol; 5.0 eq.) were mixed in ethanol (100 mL) and water (100 mL) to give a dark yellow suspension. The mixture was heated up to 90° C. for 1 h and cooled down to rt. The mixture was diluted with DCM (200 mL), stirred, filtered through Celite, and washed by DCM (3×100 mL). The combined filtrate was partitioned and the aqueous layer was extracted by DCM (100 mL). The combined DCM layers were dried over sodium sulfate and filtered through Celite again. The filtrate was concentrated down to 3-amino-5-bromo-4-chloropyridin-2-ol (1.5 g; 6.7 mmol; 20.5%) as orange powder.

7-bromo-8-chloro-1H,2H,3H-pyrido[2,3-b][1,4]oxazin-2-one: 3-amino-5-bromo-4-chloropyridin-2-ol (1.14 g; 5.1 mmol; 1.0 eq.) and potassium carbonate (2.672 g; 19.3 mmol; 3.0 eq.) were mixed with DMF (20 mL) to give a black solution. It was cooled down to 0° C. by ice bath. Chloroacetyl chloride (1.335 mL; 16.8 mmol; 2.6 eq.) was added dropwise. The mixture was stirred at room temperature for 1 h, then heated up to 92° C. for 1 h, and cooled down to rt. The mixture was diluted with DCM (40 mL), filtered through celite, and washed by 10% MeOH/DCM (3×20 mL). The brown/black suspension was dried down to a residue and purified by silica gel column 50 g, 0-20% MeOH/DCM to give 7-bromo-8-chloro-1H,2H,3H-pyrido[2,3-b][1,4]oxazin-2-one (2.1 g; 7.970 mmol; 69.02%).

7-bromo-8-chloro-1H,2H,3H-pyrido[2,3-b][1,4]oxazine: 7-bromo-8-chloro-1H,2H,3H-pyrido[2,3-b][1,4]oxazin-2-one (2.1 g; 8.0 mmol; 1.0 eq.) was mixed with borane tetrahydrofuran complex solution (1.0M in THF; 48 mL; 48 mmol; 6.0 eq.) to give a yellow suspension at room temperature. The reaction mixture was heated up to 60° C. for 4 h and then cooled down to rt. The solution was cooled by ice bath, then MeOH (30 mL) was added dropwise. The resulting mixture was stirred for 3 h at room temperature, then the mixture was concentrated to give 7-bromo-8-chloro-1H,2H,3H-pyrido[2,3-b][1,4]oxazine (1.673 g; 6.7 mmol; 84.1%) as yellow solid.

tert-butyl 7-bromo-8-chloro-1H,2H,3H-pyrido[2,3-b][1,4]oxazine-1-carboxylate: 7-bromo-8-chloro-1H,2H,3H-pyrido[2,3-b][1,4]oxazine (1.673 g; 6.7 mmol; 1.0 eq.), 4-dimethylaminopyridine (0.041 g; 0.335 mmol; 0.05 eq.) and TEA (2.804 mL; 20.1 mmol; 3.0 eq.) were added in dichloromethane (25 mL) to give a cloudy orange suspension. Di-tert-butyl dicarbonate (3.1 mL; 13.4 mmol; 2.0 eq.) was added at room temperature. The reaction mixture was heated at 30° C. for 16 h. The reaction crude was filtered and concentrated, the residue was purified by silica gel column 40 g, eluting with 20-80% EtOAc/hexanes to give tert-butyl 7-bromo-8-chloro-1H,2H,3H-pyrido[2,3-b][1,4]oxazine-1-carboxylate (1.23 g; 3.52 mmol; 52.4%).

tert-butyl 8-chloro-7-(2-{[4-(ethoxycarbonyl)-3-methylphenyl]amino}-5H,6H,7H,8H-pyrido[3,4-d]pyrimidin-7-yl)-1H,2H,3H-pyrido[2,3-b][1,4]oxazine-1-carboxylate: Tris(pentafluorophenyl)borane (0.022 g; 0.043 mmol; 0.2 eq.), ethyl 2-methyl-4-({5H,6H,7H,8H-pyrido[3,4-d]pyrimidin-2-yl}amino)benzoate (0.087 g; 0.279 mmol; 1.3 eq.), tert-butyl 7-bromo-8-chloro-1H,2H,3H-pyrido[2,3-b][1,4]oxazine-1-carboxylate (0.075 g; 0.215 mmol; 1.0 eq.) and Cesium carbonate (0.210 g; 0.644 mmol; 3.0 eq.) were added in dioxane (1.5 mL) to give a yellow suspension after stirring. The reaction was degassed with nitrogen, then Pd-PEPPSI-iPent-Cl 3-ClPy (0.009 g; 0.011 mmol; 0.050 eq.) was added in one portion and again degassed with nitrogen. The reaction mixture was heated at 110° C. for 16 h and cooled down to rt. The crude was diluted with DCM (10 mL), filtered, and washed by DCM (3×5 mL). The filtrate was concentrated and the residue was purified by silica gel column 12 g, 50-100% EtOAc/hexanes to give tert-butyl 8-chloro-7-(2-{[4-(ethoxycarbonyl)-3-methylphenyl]amino}-5H,6H,7H,8H-pyrido[3,4-d]pyrimidin-7-yl)-1H,2H,3H-pyrido[2,3-b][1,4]oxazine-1-carboxylate (62 mg; 0.107 mmol; 50%).

4-[(7-{1-[(tert-butoxy)carbonyl]-8-chloro-1H,2H,3H-pyrido[2,3-b][1,4]oxazin-7-yl}-5H,6H,7H,8H-pyrido[3,4-d]pyrimidin-2-yl)amino]-2-methylbenzoic acid: tert-Butyl 8-chloro-7-(2-{[4-(ethoxycarbonyl)-3-methylphenyl]amino}-5H,6H,7H,8H-pyrido[3,4-d]pyrimidin-7-yl)-1H,2H,3H-pyrido[2,3-b][1,4]oxazine-1-carboxylate (187 mg; 0.322 mmol; 1.0 eq.) and lithium hydroxide (116 mg; 4.827 mmol; 15 eq.) were added in methanol (3 mL), THF (1.5 mL) and water (4 mL) to give a yellow suspension, which was heated at 50° C. for 16 h. The crude was concentrated, the residue was partitioned between DCM (4×10 mL) and 10% aqueous citric acid (15 mL). The combined DCM layers were concentrated to give the crude product 4-[(7-{1-[(tert-butoxy)carbonyl]-8-chloro-1H,2H,3H-pyrido[2,3-b][1,4]oxazin-7-yl}-5H,6H,7H,8H-pyrido[3,4-d]pyrimidin-2-yl)amino]-2-methylbenzoic acid (178 mg; 0.322 mmol; 99%).

tert-butyl 8-chloro-7-(2-{[3-methyl-4-(methylcarbamoyl)phenyl]amino}-5H,6H,7H,8H-pyrido[3,4-d]pyrimidin-7-yl)-1H,2H,3H-pyrido[2,3-b][1,4]oxazine-1-carboxylate: 4-[(7-{1-[(tert-butoxy)carbonyl]-8-chloro-1H,2H,3H-pyrido[2,3-b][1,4]oxazin-7-yl}-5H,6H,7H,8H-pyrido[3,4-d]pyrimidin-2-yl)amino]-2-methylbenzoic acid (0.065 g; 0.118 mmol; 1.0 eq.) was added in DMF (1.0 mL) to give a yellow solution. It was cooled by ice bath. TEA (0.049 mL; 0.353 mmol; 3.0 eq.), 4-dimethylaminopyridine (1 mg; 0.006 mmol; 0.05 eq.) and BOP (71 mg; 0.161 mmol; 1.37 eq.) were added. Methylamine (33% wt. in ethanol) (0.073 mL; 0.588 mmol; 5.0 eq.) was added dropwise. The reaction mixture was stirred at room temperature for 15 min. The crude was partitioned between iPAc (10 mL) and sat. aqueous sodium bicarbonate (10 mL), the aqueous layer was extracted by 10 mL iPAc. The combined organic layers were dried over sodium sulfate, filtered, and concentrated. The residue was purified by silica gel column, 25 g, 60-100% EtOAc/Hex first, then 100% EtOAc to 70:18:9:3 (EtOAc/CHCl₃/MeOH/ammonia) to give tert-butyl 8-chloro-7-(2-{[3-methyl-4-(methylcarbamoyl)phenyl]amino}-5H,6H,7H,8H-pyrido[3,4-d]pyrimidin-7-yl)-1H,2H,3H-pyrido[2,3-b][1,4]oxazine-1-carboxylate (83 mg; 0.103 mmol; 54%).

4-[(7-{8-chloro-1H,2H,3H-pyrido[2,3-b][1,4]oxazin-7-yl}-5H,6H,7H,8H-pyrido[3,4-d]pyrimidin-2-yl)amino]-N,2-dimethylbenzamide: tert-Butyl 8-chloro-7-(2-{[3-methyl-4-(methylcarbamoyl)phenyl]amino}-5H,6H,7H,8H-pyrido[3,4-d]pyrimidin-7-yl)-1H,2H,3H-pyrido[2,3-b][1,4]oxazine-1-carboxylate (0.083 g; 0.147 mmol; 1.000 eq.) was added in DCM (2.000 mL; 31.201 mmol; 212.790 eq.) to give a pale yellow cloudy solution. TFA (2.000 mL; 26.924 mmol; 183.621 eq.) was added at room temperature. The mixture was stirred at room temperature for 2 h. The reaction crude was quenched into stirring toluene (20 mL), concentrated, and the residue was co-concentrated with toluene (20 mL) for 2 times. The residue was purified by silica gel column 12 g, 100% EtOAc for 5 min., then 0-5% MeOH/EtOAc to give 4-[(7-{8-chloro-1H,2H,3H-pyrido[2,3-b][1,4]oxazin-7-yl}-5H,6H,7H,8H-pyrido[3,4-d]pyrimidin-2-yl)amino]-N,2-dimethylbenzamide (20 mg; 0.041 mmol; 28%).

HPLC 95% purity, RT=2.45 min. MS: m/z=468.20, 467.15 [M+H]⁺. ¹H NMR (500 MHz, DMSO) δ 9.63 (s, 1H), 8.36 (s, 1H), 7.98 (q, J=4.6 Hz, 1H), 7.66 (dd, J=8.5, 2.2 Hz, 1H), 7.55 (d, J=2.2 Hz, 1H), 7.31 (s, 1H), 7.26 (d, J=8.4 Hz, 1H), 6.12 (d, J=2.8 Hz, 1H), 4.24 (t, J=4.4 Hz, 2H), 4.08 (s, 2H), 3.26 (t, J=5.7 Hz, 2H), 3.17 (d, J=5.2 Hz, 1H), 2.81 (t, J=5.8 Hz, 2H), 2.72 (d, J=4.5 Hz, 3H), 2.32 (s, 3H).

### Example 131: 2-{3-[(6-{8-chloro-1H,2H,3H-pyrido[2,3-b][1,4]oxazin-7-yl}-5,6,7,8-tetrahydro-2,6-naphthyridin-3-yl)amino]-5-methyl-1H-pyrazol-1-yl}-N,N-dimethylacetamide

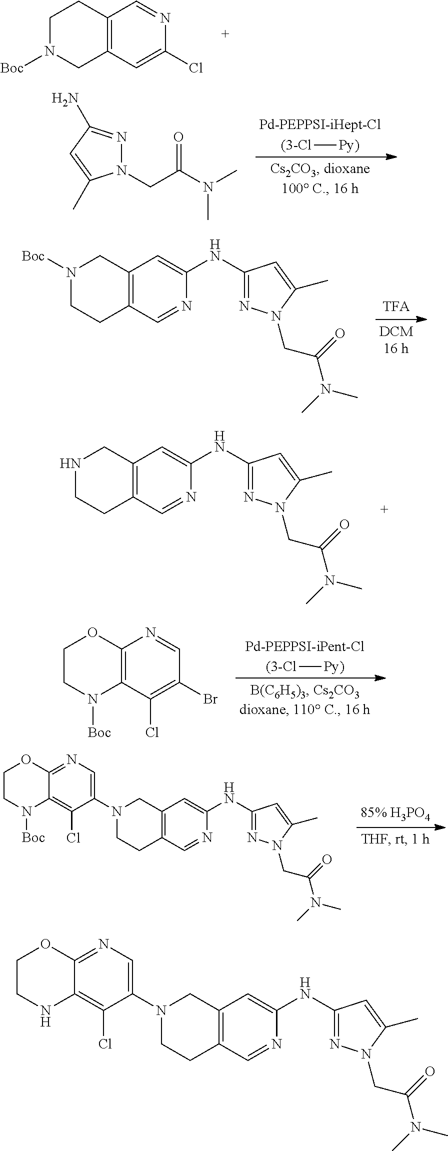

4-[(7-{8-chloro-1H,2H,3H-pyrido[2,3-b][1,4]oxazin-7-yl}-5H,6H,7H,8H-pyrido[3,4-d]pyrimidin-2-yl)amino]-N,2-dimethylbenzamide: 2-(3-Amino-5-methyl-1H-pyrazol-1-yl)-N,N-dimethylacetamide (0.240 g; 1.317 mmol; 1.000 eq.), tert-butyl 7-chloro-3,4-dihydro[2,6]naphthyridine-2(1H)-carboxylate (0.260 g; 0.967 mmol; 0.735 eq.), cesium carbonate (1.287 g; 3.951 mmol; 3.000 eq.) and some MS 4A (200 mg) were added in dioxane (4.3 mL) to give a brown suspension. The mixture was degassed with nitrogen. Pd-PEPPSI-iHept-Cl 3-ClPy (25.624 mg; 0.026 mmol; 0.020 eq.) was added in one portion. The reaction mixture was degassed with nitrogen, heated at 100° C. for 16 h, and cooled down to rt. DCM (10 mL) was added to the crude and stirred. The suspension was filtered through Celite and washed by DCM (3×6 mL). The combined DCM filtrates were concentrated and the residue was purified by silica gel column 0-35% MeOH/DCM to give tert-butyl 7-({1-[(dimethylcarbamoyl)methyl]-5-methyl-1H-pyrazol-3-yl}amino)-1,2,3,4-tetrahydro-2,6-naphthyridine-2-carboxylate (0.380 g; 0.917 mmol; 94.7%).

N,N-dimethyl-2-{5-methyl-3-[(5,6,7,8-tetrahydro-2,6-naphthyridin-3-yl)amino]-1H-pyrazol-1-yl}acetamide: DCM (6.0 mL) and TFA (6.0 mL; 80.8 mmol; 88 eq.) were added to tert-butyl 7-({1-[(dimethylcarbamoyl)methyl]-5-methyl-1H-pyrazol-3-yl}amino)-1,2,3,4-tetrahydro-2,6-naphthyridine-2-carboxylate (0.380 g; 0.917 mmol; 1.0 eq.) to form a red solution and stirred at room temperature for 16 h. Toluene (20 mL) was added and concentrated. The residue was purified by silica gel column 25 g, eluted by 100% EtOAc to 70:18:9:3 (EtOAc/CHCl3/MeOH/ammonia) to give N,N-dimethyl-2-{5-methyl-3-[(5,6,7,8-tetrahydro-2,6-naphthyridin-3-yl)amino]-1H-pyrazol-1-yl}acetamide (0.229 g; 0.728 mmol; 63%).

tert-butyl 8-chloro-7-[7-({1-[(dimethylcarbamoyl)methyl]-5-methyl-1H-pyrazol-3-yl}amino)-1,2,3,4-tetrahydro-2,6-naphthyridin-2-yl]-1H,2H,3H-pyrido[2,3-b][1,4]oxazine-1-carboxylate: N,N-dimethyl-2-{5-methyl-3-[(5,6,7,8-tetrahydro-2,6-naphthyridin-3-yl)amino]-1H-pyrazol-1-yl}acetamide (0.060 g; 0.191 mmol; 1.0 eq.), tert-butyl 7-bromo-8-chloro-1H,2H,3H-pyrido[2,3-b][1,4]oxazine-1-carboxylate (0.080 g; 0.229 mmol; 1.2 eq.), tris(pentafluorophenyl)borane (0.020 g; 0.038 mmol; 0.2 eq.) and cesium carbonate (0.187 g; 0.573 mmol; 3.0 eq.) were added in dioxane (1.2 mL) to give an orange suspension. The mixture was degassed with nitrogen, Pd-PEPPSI-iPent-Cl (3-Cl-Py) (8 mg; 0.010 mmol; 0.05 eq.) was added in one portion. The reaction mixture was degassed with nitrogen, heated at 110° C. for 16 h, and cooled down to rt. The crude was purified by silica gel column 25 g and eluted by 100% EtOAc to 70:18:9:3 (EtOAc/CHCl3/MeOH/ammonia) to give tert-butyl 8-chloro-7-[7-({1-[(dimethylcarbamoyl)methyl]-5-methyl-1H-pyrazol-3-yl}amino)-1,2,3,4-tetrahydro-2,6-naphthyridin-2-yl]-1H,2H,3H-pyrido[2,3-b][1,4]oxazine-1-carboxylate (19 mg; 0.023 mmol; 12%) as yellow powder.

2-{3-[(6-{8-chloro-1H,2H,3H-pyrido[2,3-b][1,4]oxazin-7-yl}-5,6,7,8-tetrahydro-2,6-naphthyridin-3-yl)amino]-5-methyl-1H-pyrazol-1-yl}-N,N-dimethylacetamide: tert-butyl 8-chloro-7-[7-({1-[(dimethylcarbamoyl)methyl]-5-methyl-1H-pyrazol-3-yl}amino)-1,2,3,4-tetrahydro-2,6-naphthyridin-2-yl]-1H,2H,3H-pyrido[2,3-b][1,4]oxazine-1-carboxylate (0.059 g; 0.101 mmol; 1.0 eq.) was added in THF (0.295 mL; 5 V) and cooled by ice bath. Phosphoric acid (0.885 mL; 15 V) (85%) was added. The mixture was removed from the ice bath and stirred at room temperature for 1 h. The reaction mixture was quenched by ice-cold sodium carbonate (30 mL, 10% wt.), then extracted by DCM (2×30 mL). The combined DCM was dried over sodium sulfate, filtered, and concentrated to give 2-{3-[(6-{8-chloro-1H,2H,3H-pyrido[2,3-b][1,4]oxazin-7-yl}-5,6,7,8-tetrahydro-2,6-naphthyridin-3-yl)amino]-5-methyl-1H-pyrazol-1-yl}-N,N-dimethylacetamide (12 mg; 0.026 mmol; 25.4%) as yellow solid.

HPLC 99% purity, RT=1.35 min. MS: m/z=484.20 [M+H]⁺. ¹H NMR (500 MHz, DMSO) δ 7.95 (s, 1H), 7.27 (s, 1H), 7.07 (s, 1H), 7.11 (t, J=51 Hz, 1H), 6.10 (d, J=2.9 Hz, 1H), 6.02 (s, 1H), 4.90 (s, 2H), 4.23 (t, J=4.4 Hz, 2H), 4.09 (s, 2H), 3.21 (t, J=5.7 Hz, 2H), 3.04 (s, 2H), 2.84 (s, 2H), 2.82 (d, J=5.9 Hz, 2H), 2.12 (s, 2H).

### Example 132: 7-{8-cyclopropyl-1H,2H,3H-pyrido[2,3-b][1,4]oxazin-7-yl}-N-[4-(methanesulfonylmethyl)phenyl]-5H,6H,7H,8H-pyrido[3,4-d]pyrimidin-2-amine

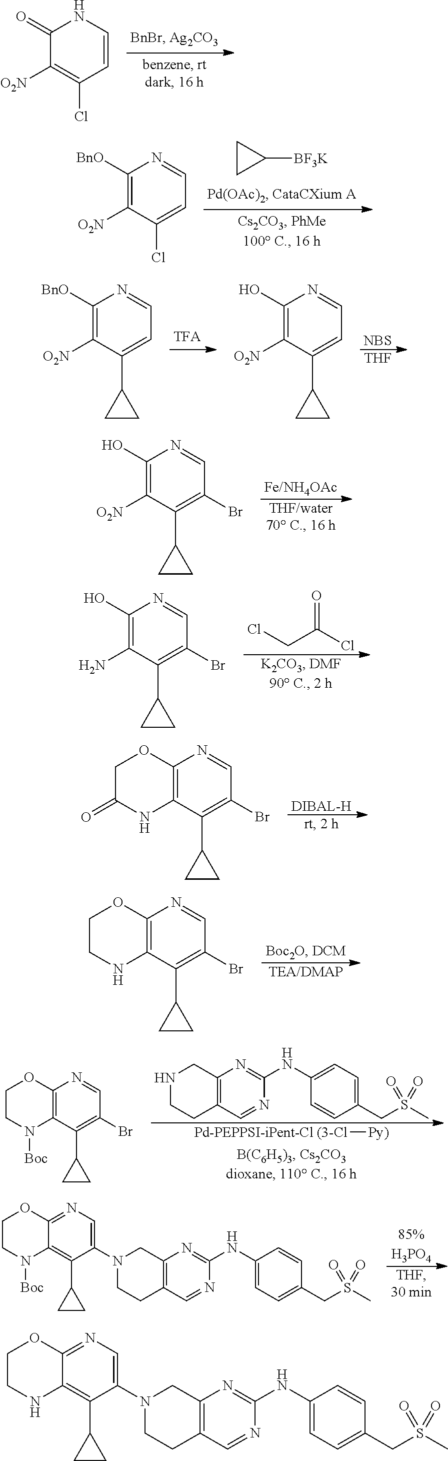

2-(benzyloxy)-4-chloro-3-nitropyridine: Silver carbonate (14.2 g; 51.6 mmol; 1.2 eq.) and 4-Chloro-2-hydroxy-3-nitropyridine (7.50 g; 43.0 mmol; 1.0 eq.) were added in benzene (150 mL) to give a yellow suspension. Benzyl bromide (6.124 mL; 51.6 mmol; 1.2 eq.) was added at room temperature. The reaction mixture was stirred in dark at room temperature for 4 days. The reaction mixture was diluted with EtOAc (200 mL), stirred and filtered through Celite, washed by EtOAc (2×50 mL), the filtrate was concentrated, the residue was purified by silica gel column 300 g, eluted by 0-40% EtOAc/Hexanes to give 2-(benzyloxy)-4-chloro-3-nitropyridine (10.51 g; 39.7 mmol; 92.4%).

2-(benzyloxy)-4-cyclopropyl-3-nitropyridine: Cesium carbonate (22.159 g; 68.011 mmol; 3.0 eq.) was added in water (24 mL), it was stirred at room temperature for 15 min, then toluene (100 mL) was added, followed by addition of di(1-adamantyl)-n-butylphosphine (0.244 g; 0.680 mmol; 0.03 eq.), 2-(benzyloxy)-4-chloro-3-nitropyridine (6.0 g; 22.670 mmol; 1.0 eq.) and potassium cyclopropyltrifluoroborate (4.937 g; 45.341 mmol; 2.0 eq.), the yellow mixture was degassed with Nitrogen. Palladium(ii)acetate (0.102 g; 0.453 mmol; 0.02 eq.) was added. The reaction mixture was degassed with nitrogen, heated up to 110° C. for 16 h, and cooled down to rt. EtOAc (100 mL) and water (50 mL) were added to the reaction mixture and partitioned. The organic layer was washed by 20% brine (40 mL), dried over sodium sulfate, filtered, and concentrated to give 2-(benzyloxy)-4-cyclopropyl-3-nitropyridine (6.12 g; 25.9 mmol; 99%) as brown oil.

4-cyclopropyl-3-nitropyridin-2-ol: Cooled by ice bath, to the solid 2-(benzyloxy)-4-cyclopropyl-3-nitropyridine (6.127 g; 22.670 mmol; 1.0 eq.) was added TFA (13 mL) slowly. The mixture was stirred with cooling for 1 h then at room temperature for 16 h. Toluene (60 mL) was added to the reaction mixture, then it was concentrated down to give 4-cyclopropyl-3-nitropyridin-2-ol.

5-bromo-4-cyclopropyl-3-nitropyridin-2-ol: A solution of 4-cyclopropyl-3-nitropyridin-2-ol (5.570 g; 20.714 mmol; 1.0 eq.) in THF (60 mL) was cooled by ice bath. N-Bromosuccinimide (4.793 g; 26.928 mmol; 1.3 eq.) was added in one portion. The mixture was stirred at room temperature for 16 h. Concentrated, the residue was purified by silica gel column 225 g, and eluted by 40-90% EtOAc/Hexanes to give about 6 g yellow solid, which was slurred in water (60 mL) at 50° C. for 1 h, then filtered. The collected cake was washed by water and dried in vacuum oven to give 5-bromo-4-cyclopropyl-3-nitropyridin-2-ol (2.9 g; 11.19 mmol; 54%).

3-amino-5-bromo-4-cyclopropylpyridin-2-ol: Ammonium chloride (2.684 g; 50.182 mmol; 5.0 eq.), iron (2.802 g; 50.182 mmol; 5.0 eq.) and 5-bromo-4-cyclopropyl-3-nitropyridin-2-ol (2.6 g; 10.036 mmol; 1.0 eq.) were added in THF (62.4 mL) and water (31.2 mL), and the mixture was heated at 70° C. for 16 h. The reaction mixture was concentrated. EtOAc (120 mL) and acetonitrile (12 mL) were added to the residue. The mixture was heated up to 70° C. for 16 h, cooled down to 45° C., filtered, and washed by EtOAc (3×20 mL). The filtrate was concentrated to give 3-amino-5-bromo-4-cyclopropylpyridin-2-ol (2.04 g; 8.905 mmol; 88.7%).

7-bromo-8-cyclopropyl-1H,2H,3H-pyrido[2,3-b][1,4]oxazin-2-one: 3-Amino-5-bromo-4-cyclopropylpyridin-2-ol (2.040 g; 8.905 mmol; 1.0 eq.) and potassium carbonate (3.692 g; 26.716 mmol; 3.0 eq.) were added in DMF (40.8 mL). The mixture was cooled down to 0° C. by ice bath. Chloroacetyl chloride (2.128 mL; 26.716 mmol; 3.0 eq.) was added dropwise, then the mixture was removed from the ice bath. The mixture was heated at 95° C. for about 75 min. and cooled down to rt. The crude was filtered and concentrated. The residue was purified by silica gel column 100 g and eluted by 0-10% MeOH/DCM to give 7-bromo-8-cyclopropyl-1H,2H,3H-pyrido[2,3-b][1,4]oxazin-2-one (2.260 g; 8.399 mmol; 94.3%) as orange solid.

7-bromo-8-cyclopropyl-1H,2H,3H-pyrido[2,3-b][1,4]oxazine: Cooled by ice-bath, diisobutylaluminum hydride (1.0 M in toluene) (41.8 mL; 41.8 mmol; 7.0 eq.) was added to 7-bromo-8-cyclopropyl-1H,2H,3H-pyrido[2,3-b][1,4]oxazin-2-one (1.607 g; 5.972 mmol; 1.0 eq.) to give a deep red solution. The mixture was stirred with the cooling bath for 10 min. then stirred at room temperature for about 2 h. The reaction crude was added into stirring ice-cold 10% Rochella salt (250 mL, 25 g), then Me-THF (20 mL) and EtOAc (100 mL) were added, stirred and partitioned. The organic layer was washed by brine and dried over sodium sulfate, filtered, and concentrated to give 7-bromo-8-cyclopropyl-1H,2H,3H-pyrido[2,3-b][1,4]oxazine (1.3 g; 5.096 mmol; 85.3%).

tert-butyl 7-bromo-8-cyclopropyl-1H,2H,3H-pyrido[2,3-b][1,4]oxazine-1-carboxylate: 7-bromo-8-cyclopropyl-1H,2H,3H-pyrido[2,3-b][1,4]oxazine (1.3 g; 5.096 mmol; 1.0 eq.), 4-dimethylaminopyridine (0.031 g; 0.255 mmol; 0.05 eq.) and TEA (2.131 mL; 15.287 mmol; 3.0 eq.) were added in DCE (22 mL). Di-tert-butyl dicarbonate (2.575 mL; 11.211 mmol; 2.2 eq.) was added at room temperature. The mixture was heated at 75° C. for 16 h. The reaction mixture was filtered through celite and concentrated. The residue was purified by silica gel column 80 g and eluted by 0-60% EA/Hexanes to give tert-butyl 7-bromo-8-cyclopropyl-1H,2H,3H-pyrido[2,3-b][1,4]oxazine-1-carboxylate (530 mg; 1.492 mmol; 29.3%).

tert-butyl 8-cyclopropyl-7-(2-{[4-(methanesulfonylmethyl)phenyl]amino}-5H,6H,7H,8H-pyrido[3,4-d]pyrimidin-7-yl)-1H,2H,3H-pyrido[2,3-b][1,4]oxazine-1-carboxylate: tert-butyl 7-bromo-8-cyclopropyl-1H,2H,3H-pyrido[2,3-b][1,4]oxazine-1-carboxylate (0.070 g; 0.197 mmol; 1.0 eq.), N-[4-(methanesulfonylmethyl)phenyl]-5H,6H,7H,8H-pyrido[3,4-d]pyrimidin-2-amine (0.075 g; 0.236 mmol; 1.2 eq.), tris(pentafluorophenyl)borane, 95% (0.020 g; 0.039 mmol; 0.2 eq.), cesium carbonate (0.193 g; 0.591 mmol; 3.0 eq.) and MS 4A (100 mg) were added in dioxane (1.12 mL) to give a yellow suspension after stirring. The mixture was degassed with nitrogen, Pd-PEPPSI-iPent-Cl (3-Cl-Py) (0.017 g; 0.020 mmol; 0.1 eq.). The reaction mixture was degassed with nitrogen and heated at 110° C. for 22 h. It was cooled down to rt. The crude was purified by silica gel column 25 g and eluted by 50-100% EtOAc/Hexanes to give tert-butyl 8-cyclopropyl-7-(2-{[4-(methanesulfonylmethyl)phenyl]amino}-5H,6H,7H,8H-pyrido[3,4-d]pyrimidin-7-yl)-1H,2H,3H-pyrido[2,3-b][1,4]oxazine-1-carboxylate (0.016 g; 0.027 mmol; 13.7%) as yellow solid.

7-{8-cyclopropyl-1H,2H,3H-pyrido[2,3-b][1,4]oxazin-7-yl}-N-[4-(methanesulfonylmethyl)phenyl]-5H,6H,7H,8H-pyrido[3,4-d]pyrimidin-2-amine: tert-butyl 8-cyclopropyl-7-(2-{[4-(methanesulfonylmethyl)phenyl]amino}-5H,6H,7H,8H-pyrido[3,4-d]pyrimidin-7-yl)-1H,2H,3H-pyrido[2,3-b][1,4]oxazine-1-carboxylate (0.064 g; 0.108 mmol; 1.0 eq.) in THF (0.32 mL) to give a yellow suspension. The mixture was cooled by ice bath and phosphoric acid (0.96 mL) (85%) was added. Then the ice bath was removed and the mixture was stirred at room temperature for 40 min. The reaction mixture was quenched by DCM (10 mL) and ice-cold water (15 g). The DCM layer was discarded, the aqueous layer was basified by aqueous sodium carbonate (10%, 28 mL), and extracted by DCM (3×40 mL). The DCM layers were dried over sodium sulfate, filtered, and concentrated to give an orange solid. The 25 mg solid was dissolved in DMSO, purified by 55 g C18 column and eluted by water with 0.1% ammonia and acetonitrile to give 7-{8-cyclopropyl-1H,2H,3H-pyrido[2,3-b][1,4]oxazin-7-yl}-N-[4-(methanesulfonylmethyl)phenyl]-5H,6H,7H,8H-pyrido[3,4-d]pyrimidin-2-amine (7 mg; 0.013 mmol; 14.6%) as yellow solid.

HPLC 94% purity, RT=2.77 min. MS: m/z=493.15 [M+H]⁺. ¹H NMR (500 MHz, DMSO) δ 9.64 (s, 1H), 8.35 (s, 1H), 7.77 (d, J=8.3 Hz, 2H), 7.28 (d, J=8.3 Hz, 2H), 7.15 (s, 1H), 4.37 (s, 1H), 4.19 (t, J=4.4 Hz, 1H), 4.06 (s, 1H), 1.46 (td, J=8.2, 4.2 Hz, 1H), 1.23 (s, 2H), 0.97 (d, J=7.7 Hz, 2H), 0.59 (d, J=5.3 Hz, 2H).

### Example 133: Synthesis of {4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}methanol

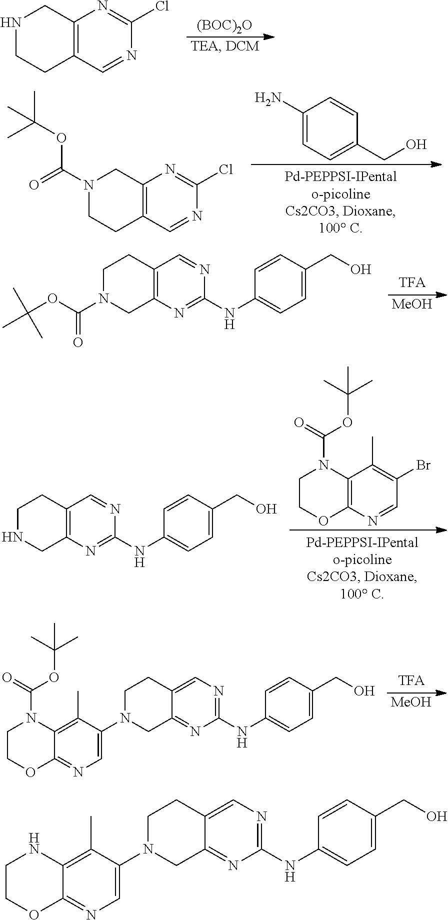

tert-butyl 2-{[4-(hydroxymethyl)phenyl]amino}-5H,6H,7H,8H-pyrido[3,4-d]pyrimidine-7-carboxylate: In a 40 mL vial were added tert-butyl 2-chloro-5H,6H,7H,8H-pyrido[3,4-d]pyrimidine-7-carboxylate (0.70 g; 2.60 mmol; 1.00 eq.), (4-aminophenyl)methanol (0.38 g; 3.11 mmol; 1.20 eq.), cesium carbonate (2.54 g; 7.79 mmol; 3.00 eq.) in dioxane (20 mL) to give a suspension. The brown mixture was degassed with Argon for 5 minutes, then 2-methylpyridine; {1,3-bis[2,6-bis(pentan-3-yl)phenyl]-4,5-dichloro-2,3-dihydro-1H-imidazol-2-ylidene}dichloropalladium (109.02 mg; 0.13 mmol; 0.05 eq.) was added and capped. The mixture was heated at 100° C. for 16 h. LCMS showed the reaction was complete and desired product was observed. The insoluble was removed by filtration. The solvent was concentrated and to get tert-butyl 2-{[4-(hydroxymethyl)phenyl]amino}-5H,6H,7H,8H-pyrido[3,4-d]pyrimidine-7-carboxylate (1300.00 mg; crude product) as pale yellow powder, used as in the next step. MS: m/z 357 (M+H).

[4-({4aH,5H,6H,7H,8H,8aH-pyrido[3,4-d]pyrimidin-2-yl}amino)phenyl]methanol: To a suspension of tert-butyl 2-{[4-(hydroxymethyl)phenyl]amino}-5H,6H,7H,8H-pyrido[3,4-d]pyrimidine-7-carboxylate (1.30 g; 3.65 mmol; 1.00 eq.) in MeOH (2 mL) was added trifluoroacetic acid (13.96 ml; 182.37 mmol; 50.00 eq.). The mixture was stirred for 15 minutes. LCMS showed the reaction was complete and desired product was observed. Product purified via reverse phase column chromatography. The solvent was concentrated to get [4-({4aH,5H,6H,7H,8H,8aH-pyrido[3,4-d]pyrimidin-2-yl}amino)phenyl]methanol (753.00 mg; 2.92 mmol; 79.9%) a dark brown grease used in next step of synthesis. MS: m/z 259 (M+H).

tert-butyl 7-(2-{[4-(hydroxymethyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate: In a 40 mL vial were added [4-({4aH,5H,6H,7H,8H,8aH-pyrido[3,4-d]pyrimidin-2-yl}amino)phenyl]methanol (230.00 mg; 0.89 mmol; 1.00 eq.), dicaesium(1+) carbonate (870.30 mg; 2.67 mmol; 3.00 eq.) in dioxane (20 mL) to give a suspension. The mixture was degassed with Argon for 5 minutes, then tert-butyl 7-bromo-8-methyl-1H,2H,3H-pyrido[2,3-b][1,4]oxazine-1-carboxylate (351.72 mg; 1.07 mmol; 1.20 eq.) and 2-methylpyridine; {1,3-bis[2,6-bis(pentan-3-yl)phenyl]-4,5-dichloro-2,3-dihydro-1H-imidazol-2-ylidene}dichloropalladium (22.44 mg; 0.03 mmol; 0.03 eq.) was added and capped. The mixture was heated at 100° C. for 16 h. LCMS showed the reaction was complete and desired product was observed. The insoluble was removed by filtration. And purified on Interchim. The solvent was concentrated and to tert-butyl 7-(2-{[4-(hydroxymethyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (385.00 mg; 0.76 mmol; 85.7%) as pale yellow powder, used as is in the next step. MS: m/z 505 (M+H).

{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}methanol: To a suspension of tert-butyl 7-(2-{[4-(hydroxymethyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (385.00 mg; 0.76 mmol; 1.00 eq.) in methanol (6.93 ml; 18.00 V) was added trifluoroacetic acid (2.92 ml; 38.15 mmol; 50.00 eq.). The mixture was stirred for 15 minutes. LCMS showed the reaction was complete and desired product was observed. Product purified via reverse phase column chromatography, UPLC showed product purity of 92%. The solvent was frozen and lyophilized to get {4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}methanol (43.40 mg; 0.11 mmol; 14.1%) a light yellow powder. MS: m/z 405 (M+H). ¹H NMR (500 MHz, DMSO) δ 10.11 (s, 1H), 9.74 (s, 1H), 8.38 (s, 1H), 7.93 (d, J=8.3 Hz, 2H), 7.74 (d, J=8.3 Hz, 2H), 7.23 (s, 1H), 5.47 (s, 1H), 4.12 (s, 2H), 3.95 (s, 2H), 3.23 (m, 4H), 3.06 (d, J=6.0 Hz, 2H), 2.76 (d, J=6.2 Hz, 2H), 1.98 (s, 3H).

### Example 134: Synthesis of 2-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}ethan-1-ol

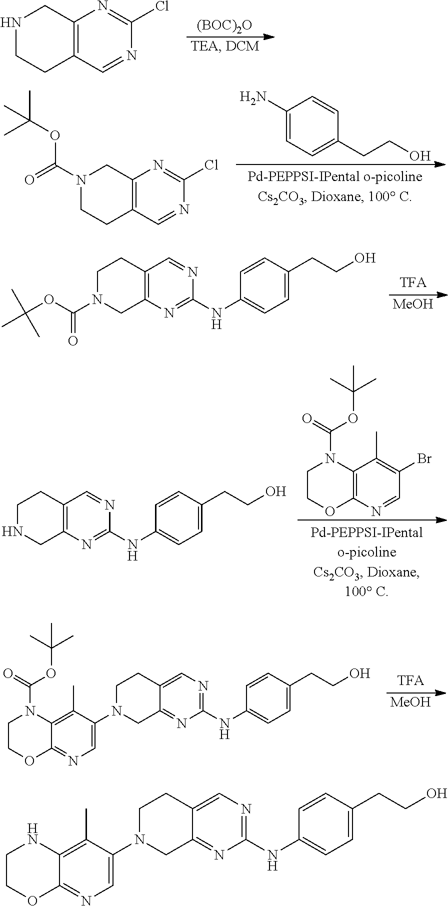

tert-butyl 2-{[4-(2-hydroxyethyl)phenyl]amino}-5H,6H,7H,8H-pyrido[3,4-d]pyrimidine-7-carboxylate: In a 40 mL vial were added tert-butyl 2-chloro-5H,6H,7H,8H-pyrido[3,4-d]pyrimidine-7-carboxylate (0.70 g; 2.60 mmol; 1.00 eq.), 2-(4-aminophenyl)ethan-1-ol (0.43 g; 3.11 mmol; 1.20 eq.), cesium carbonate (2.54 g; 7.79 mmol; 3.00 eq.) in dioxane (20 mL) to give a suspension.

The brown mixture was degassed with Argon for 5 minutes, then 2-methylpyridine; {1,3-bis[2,6-bis(pentan-3-yl)phenyl]-4,5-dichloro-2,3-dihydro-1H-imidazol-2-ylidene}dichloropalladium (109.02 mg; 0.13 mmol; 0.05 eq.) was added and capped. The mixture was heated at 100° C. for 16 h. LCMS showed the reaction was complete and desired product was observed. The insoluble was removed by filtration. The solvent was concentrated and to get tert-butyl 2-{[4-(2-hydroxyethyl)phenyl]amino}-5H,6H,7H,8H-pyrido[3,4-d]pyrimidine-7-carboxylate (1300.00 mg; crude product) as pale yellow powder, used as is in the next step. MS: m/z 371 (M+H).

2-[4-({5H,6H,7H,8H-pyrido[3,4-d]pyrimidin-2-yl}amino)phenyl]ethan-1-ol: In a 100 mL round bottom flask were added tert-butyl 2-{[4-(2-hydroxyethyl)phenyl]amino}-5H,6H,7H,8H-pyrido[3,4-d]pyrimidine-7-carboxylate (0.96 g; 2.60 mmol; 1.00 eq.) and trifluoroacetic acid (1.20 eq.) in methanol (2.00 ml; 2.08 V). The mixture was stirred at room temperature at for 20 minutes. LCMS showed the reaction was complete and desired product was observed. The product was purified via reverse phase column chromatography, and solvent concentrated to produce a dark brown grease (727 mg, 102%). MS: m/z 273 (M+H).

tert-butyl 7-(2-{[4-(2-hydroxyethyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate: In a 40 mL vial were added 2-[4-({5H,6H,7H,8H-pyrido[3,4-d]pyrimidin-2-yl}amino)phenyl]ethan-1-ol (343.00 mg; 1.27 mmol; 1.00 eq.) and tert-butyl 7-bromo-8-methyl-1H,2H,3H-pyrido[2,3-b][1,4]oxazine-1-carboxylate (501.22 mg; 1.52 mmol; 1.20 eq.) in dioxane (20 mL) to give a suspension. The mixture was degassed with Argon for 5 minutes, then 2-methylpyridine; {1,3-bis[2,6-bis(pentan-3-yl)phenyl]-4,5-dichloro-2,3-dihydro-1H-imidazol-2-ylidene}dichloropalladium (31.98 mg; 0.04 mmol; 0.03 eq.) and dicaesium(1+) carbonate (1240.22 mg; 3.81 mmol; 3.00 eq.) was added and capped. The mixture was heated at 100° C. for 16 h. LCMS showed the reaction was complete and desired product was observed. The insoluble was removed by filtration. The solvent was concentrated to get tert-butyl 7-(2-{[4-(2-hydroxyethyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (101.00 mg; 0.19 mmol; 15.3%) and purified under basic conditions via Interchim reverse phase column. Product is a thick red grease used in the next step. MS: m/z 519 (M+H).

2-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}ethan-1-ol: To a suspension of tert-butyl 7-(2-{[4-(2-hydroxyethyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (101.00 mg; 0.19 mmol; 1.00 eq.) in methanol (1.00 ml) was added trifluoroacetic acid (0.75 ml; 9.74 mmol; 50.00 eq.). The mixture was stirred for 15 minutes. LCMS showed the reaction was complete and desired product was observed. Product purified via reverse phase column chromatography, UPLC showed product purity of 92%. The solvent was frozen and lyophilized to get 2-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}ethan-1-ol (14.90 mg; 0.03 mmol; 16.6%) a light yellow powder. MS: m/z 419 (M+H). ¹H NMR (500 MHz, DMSO) δ 9.39 (s, 1H), 8.29 (s, 1H), 7.61 (t, J=9.3 Hz, 2H), 7.32-7.03 (m, 3H), 6.97 (d, J=8.3 Hz, 1H), 5.52 (s, 1H), 4.58 (s, 1H), 4.32-3.65 (m, 4H), 3.53 (d, J=7.9 Hz, 2H), 3.11 (d, J=6.0 Hz, 2H), 2.90 (t, J=8.5 Hz, 2H), 2.84-2.70 (m, 2H), 2.64 (t, J=7.4 Hz, 2H), 2.02 (s, 3H).

### Example 135: Synthesis of Compound N-{4-[(2-methoxyethanesulfonyl) methyl]phenyl}-7-{8-methyl-1H,2H,3H-pyrido[2,3-b][1,4]oxazin-7-yl}-5H,6H,7H,8H-pyrido[3,4-d]pyrimidin-2-amine

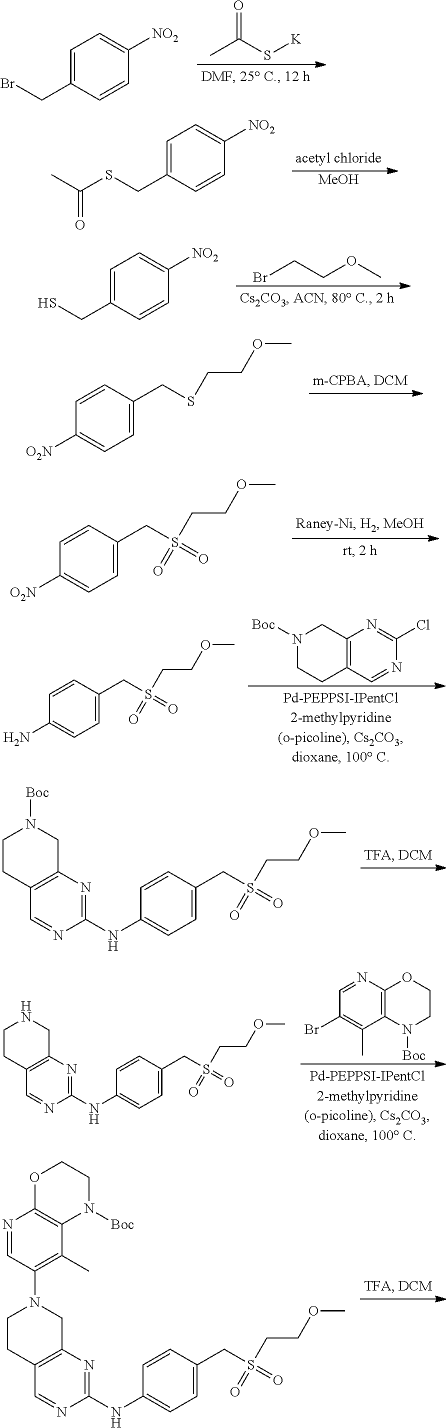

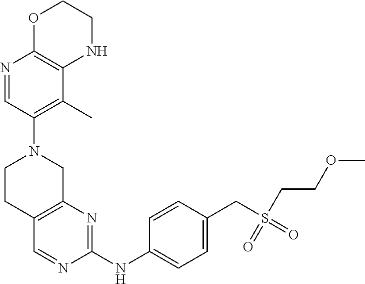 [SMILES: COCCS(=O)(=O)Cc1ccc(Nc2ncc3c(n2)CN(c2cnc4c(c2C)NCCO4)CC3)cc1]

1-{[(4-nitrophenyl)methyl]sulfanyl}ethan-1-one: To a stirred solution of 1-(bromomethyl)-4-nitrobenzene (5.00 g; 21.99 mmol) in DMF (50.00 ml) was added 1-(potassiosulfanyl) ethan-1-one (3.17 g; 26.38 mmol) in portions at room temperature. After stirring for 16 h at 25° C. The resulting mixture was extracted with EtOAc. The combined organic layers were washed with NaCl(aq.), dried over anhydrous Na₂SO₄. After filtration, the filtrate was concentrated under reduced pressure to afford 1-{[(4-nitrophenyl)methyl] sulfanyl}ethan-1-one (4.60 g; 98.50%) as a black solid.

(4-nitrophenyl) methanethiol: To a stirred solution of 1-{[(4-nitrophenyl)methyl]sulfanyl}ethan-1-one (4.60 g; 21.15 mmol) in MeOH (20.00 ml) was added acetyl chloride (2.00 ml) in portion at room temperature. After stirring for 16 h at 30° C. The resulting mixture was concentrated under reduced pressure. The residue was purified by silica gel column chromatography, eluted with PE/EA (10:1) to afford (4-nitrophenyl) methanethiol (3.30 g; 87.71%) as a white solid.

1-{[(2-methoxyethyl) sulfanyl] methyl}-4-nitrobenzene: To a stirred solution of (4-nitrophenyl) methanethiol (1.50 g; 8.80 mmol) and 1-bromo-2-methoxyethane (1.54 g; 10.56 mmol) in ACN (10.00 ml) was added Cs₂CO₃ (6.04 g; 17.60 mmol) in portion at room temperature. After stirring for 3 h at 80° C. The resulting mixture was concentrated under reduced pressure. The residue was purified by silica gel column chromatography, eluted with PE/EA (10:1) to afford 1-{[(2-methoxyethyl) sulfanyl] methyl}-4-nitrobenzene (770.00 mg; 38.15%) as a yellow oil.

1-[(2-methoxyethanesulfonyl)methyl]-4-nitrobenzene: To a stirred solution of 1-{[(2-methoxyethyl) sulfanyl] methyl}-4-nitrobenzene (770.00 mg; 3.36 mmol) in DCM (12.00 ml) was added m-CPBA (2439.51 mg; 13.43 mmol). The resulting mixture was stirred for 2 h at 25° C. under nitrogen atmosphere. The resulting mixture was concentrated under reduced pressure. The residue was purified by silica gel column chromatography, eluted with PE/EA (1:1) to afford 1-[(2-methoxyethanesulfonyl) methyl]-4-nitrobenzene (770.00 mg; 87.40%) as a white solid.

4-[(2-methoxyethanesulfonyl) methyl]aniline: To a solution of 1-[(2-methoxyethanesulfonyl)methyl]-4-nitrobenzene (770.00 mg; 2.94 mmol) in MeOH (20.00 ml) was added Raney-Ni (305.00 mg; 3.38 mmol) at room temperature. After stirring for 1 h at room temperature under hydrogen atmosphere. The resulting mixture was filtered, the filter cake was washed with MeOH and then filtrate was concentrated under reduced pressure to afford 4-[(2-methoxyethanesulfonyl) methyl]aniline (580.00 mg 85.51%) as a white solid.

tert-butyl 2-({4-[(2-methoxyethanesulfonyl)methyl]phenyl}amino)-5H,6H,7H,8H-pyrido[3,4-d]pyrimidine-7-carboxylate: To a stirred solution of 4-[(2-methoxyethanesulfonyl) methyl]aniline (409.05 mg; 1.76 mmol) and tert-butyl 2-chloro-5H,6H,7H,8H-pyrido[3,4-d]pyrimidine-7-carboxylate (500.00 mg; 1.76 mmol) in Dioxane-1,4 (10.00 ml) were added Pd-PEPPSI-IPentCl 2-methylpyridine (o-picoline) (155.89 mg; 0.18 mmol) and Cs₂CO₃ (1209.10 mg; 3.53 mmol) at room temperature. The resulting mixture was stirred for 3 h at 100° C. under nitrogen atmosphere. The resulting mixture was concentrated under reduced pressure. The residue was purified by silica gel column chromatography, eluted with PE/EA (1:1) to afford tert-butyl 2-({4-[(2-methoxyethanesulfonyl) methyl]phenyl}amino)-5H,6H,7H,8H-pyrido[3,4-d]pyrimidine-7-carboxylate (720.00 mg; 87.79%) as a yellow solid.

N-{4-[(2-methoxyethanesulfonyl) methyl]phenyl}-5H,6H,7H,8H-pyrido[3,4-d]pyrimidin-2-amine: To a stirred solution of tert-butyl 2-({4-[(2-methoxyethanesulfonyl) methyl]phenyl}amino)-5H,6H,7H,8H-pyrido[3,4-d]pyrimidine-7-carboxylate (700.00 mg; 1.50 mmol) in DCM (15.00 ml) was added TFA (3.00 ml) in portion at room temperature. After stirring for 1 h at room temperature. The mixture was basified to pH [7] with saturated NaHCO₃ (aq.). The resulting mixture was extracted with EtOAc. The combined organic layers were washed with NaCl(aq.), dried over anhydrous Na₂SO₄. After filtration, the filtrate was concentrated under reduced pressure to afford N-{4-[(2-methoxyethanesulfonyl) methyl]phenyl}-5H,6H,7H,8H-pyrido[3,4-d]pyrimidin-2-amine (480.00 mg; Crude Product) as a yellow solid.

tert-butyl 7-[2-({4-[(2-methoxyethanesulfonyl) methyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate: To a stirred solution of N-{4-[(2-methoxyethanesulfonyl)methyl]phenyl}-5H,6H,7H,8H-pyrido[3,4-d]pyrimidin-2-amine (427.03 mg; 1.16 mmol) and tert-butyl 7-bromo-8-methyl-1H,2H,3H-pyrido[2,3-b][1,4]oxazine-1-carboxylate (400.00 mg; 1.16 mmol) in Dioxane-1,4 (15.00 ml) was added Pd-PEPPSI-IPentCl 2-methylpyridine (o-picoline) (102.18 mg; 0.12 mmol) and Cs₂CO₃ (792.56 mg; 2.31 mmol) at room temperature. The resulting mixture was stirred for 3 h at 100° C. under nitrogen atmosphere. The resulting mixture was concentrated under reduced pressure. The residue was purified by silica gel column chromatography, eluted with PE/EA (1:100) to afford tert-butyl 7-[2-({4-[(2-methoxyethanesulfonyl) methyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate (300.00 mg; 38.00%) as a yellow solid.

N-{4-[(2-methoxyethanesulfonyl) methyl]phenyl}-7-{8-methyl-1H,2H,3H-pyrido[2,3-b][1,4]oxazin-7-yl}-5H,6H,7H,8H-pyrido[3,4-d]pyrimidin-2-amine: To a stirred solution of tert-butyl 7-[2-({4-[(2-methoxyethanesulfonyl) methyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate (150.00 mg; 0.22 mmol) in DCM (10.00 ml) was added TFA (2.00 ml) in portions at room temperature. After stirring for 1 h at room temperature. The resulting mixture was concentrated under reduced pressure. The crude product was purified by Prep-HPLC with following conditions Column: XBridge Prep OBD C18 Column, 30*150 mm, 5 μm; Mobile Phase A: Water (10 mmol NH₄HCO₃+0.05% NH₄OH), Mobile Phase B: ACN; Flow rate: 60 mL/min; Gradient: 25% B to 52% B in 8 min, Wavelength: 254 nm; RT1 (min): 7 to afford N-{4-[(2-methoxyethanesulfonyl) methyl]phenyl}-7-{8-methyl-1H,2H,3H-pyrido[2,3-b][1,4]oxazin-7-yl}-5H,6H,7H,8H-pyrido[3,4-d]pyrimidin-2-amine (70.30 mg 62.47%) as a white solid,

HPLC: 99.6% purity, RT=2.98 min. MS: m/z=511.10 [M+H]⁺. ¹H NMR (300 MHz, DMSO-d6): 9.63 (s, 1H), 8.36 (s, 1H), 7.78 (d, J=8.6 Hz, 2H), 7.33-7.22 (m, 3H), 5.54 (s, 1H), 4.36 (s, 2H), 4.21 (d, J=4.6 Hz, 2H), 3.98 (s, 2H), 3.71 (t, J=5.8 Hz, 2H), 3.58 (s, 2H), 3.26 (d, J=5.8 Hz, 5H), 3.13 (s, 2H), 2.81 (s, 2H), 2.05 (s, 3H).

### Example 136: Synthesis of Compound 7-{imidazo[1,2-a]pyrazin-3-yl}-N-[4-(methanesulfonylmethyl)phenyl]-5H,6H,7H,8H-pyrido[3,4-d]pyrimidin-2-amine

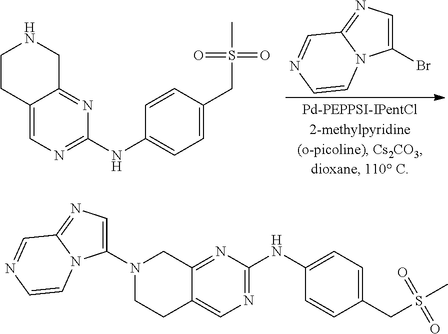

7-{imidazo [1,2-a] pyrazin-3-yl}-N-[4-(methanesulfonylmethyl) phenyl]-5H,6H,7H,8H-pyrido[3,4-d]pyrimidin-2-amine: To a stirred solution of 3-bromoimidazo[1,2-a] pyrazine (367.74 mg; 1.79 mmol) and N-[4-(methanesulfonylmethyl)phenyl]-5H,6H,7H,8H-pyrido[3,4-d]pyrimidin-2-amine (500.00 mg; 1.49 mmol) in Dioxane-1,4 (12.00 ml) was added Pd-PEPPSI-IPentCl 2-methylpyridine (o-picoline) (66.03 mg; 0.07 mmol) and Cs₂CO₃ (1536.43 mg; 4.48 mmol) at room temperature. The resulting mixture was stirred for 3 h at 105° C. under nitrogen atmosphere. The resulting mixture was concentrated under reduced pressure. The crude product was purified by Prep-HPLC with following conditions Column: XBridge Prep OBD C18 Column, 30*150 mm, 5 μm; Mobile Phase A: Water (10 mmol NH₄HCO₃+0.05% NH₄OH), Mobile Phase B: ACN; Flow rate: 60 mL/min; Gradient: 25% B to 55% B in 8 min, Wavelength: 254 nm; RT1 (min): 7 to afford 7-{imidazo[1,2-a]pyrazin-3-yl}-N-[4-(methanesulfonylmethyl)phenyl]-5H,6H,7H,8H-pyrido[3,4-d]pyrimidin-2-amine (10.10 mg; 1.42%) as a white solid.

HPLC: 91.63% purity, RT=2.25 min. MS: m/z=436.05 [M+H]⁺. ¹H NMR (300 MHz, DMSO-d6): 9.68 (s, 1H), 9.00 (d, J=1.4 Hz, 1H), 8.42 (s, 1H), 8.33 (dd, J=4.7, 1.5 Hz, 1H), 7.88 (d, J=4.6 Hz, 1H), 7.79 (d, J=8.5 Hz, 2H), 7.65 (s, 1H), 7.29 (d, J=8.5 Hz, 2H), 4.38 (s, 2H), 4.28 (s, 2H), 3.52 (s, 2H), 2.94 (s, 2H), 2.87 (s, 3H).

### Example 137: Synthesis of Compound N-(4-{[2-(2-methoxyethoxy) ethanesulfonyl] methyl}phenyl)-7-{8-methyl-1H,2H,3H-pyrido[2,3-b][1,4]oxazin-7-yl}-5H,6H,7H,8H-pyrido[3,4-d]pyrimidin-2-amine

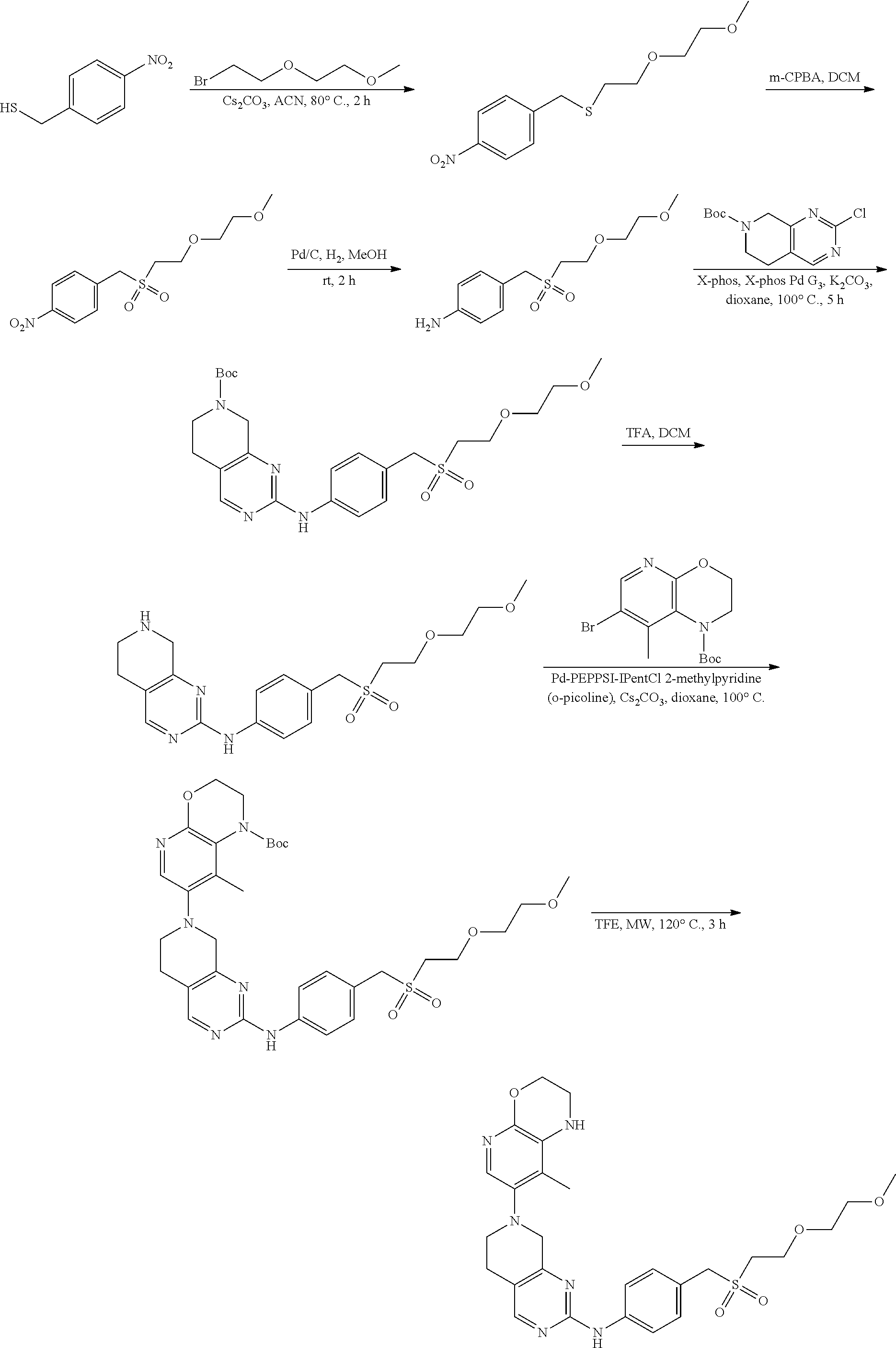

1-({[2-(2-methoxyethoxy)ethyl] sulfanyl}methyl)-4-nitrobenzene: To a stirred solution of (4-nitrophenyl) methanethiol (1.50 g; 8.54 mmol) and 1-bromo-2-(2-methoxyethoxy) ethane (1.90 g; 10.25 mmol) in ACN (30.00 ml) was added Cs₂CO₃ (5.86 g; 17.08 mmol) in portions at room temperature. After stirring for 3 h at 80° C. The resulting mixture was concentrated under reduced pressure. The residue was purified by silica gel column chromatography, eluted with PE/EA (6:1) to afford 1-({[2-(2-methoxyethoxy)ethyl]sulfanyl}methyl)-4-nitrobenzene (640.00 mg; 24.85%) as a yellow oil.

1-{[2-(2-methoxyethoxy) ethanesulfonyl] methyl}-4-nitrobenzene: To a stirred solution of 1-({[2-(2-methoxyethoxy)ethyl] sulfanyl}methyl)-4-nitrobenzene (580.00 mg; 1.92 mmol) in DCM (15.00 ml) was added m-CPBA (1397.88 mg; 7.70 mmol). The resulting mixture was stirred for 5 h at room temperature under nitrogen atmosphere. The resulting mixture was concentrated under reduced pressure. The residue was purified by silica gel column chromatography, eluted with PE/EA (1:1) to afford 1-{[2-(2-methoxyethoxy) ethanesulfonyl] methyl}-4-nitrobenzene (410.00 mg 69.84%) as a white solid.

4-{[2-(2-methoxyethoxy) ethanesulfonyl] methyl}aniline: To a solution of 1-{[2-(2-methoxyethoxy) ethanesulfonyl] methyl}-4-nitrobenzene (380.00 mg; 1.25 mmol) in MeOH (20.00 ml) was added Pd/C (132.52 mg; 0.12 mmol;) at room temperature. The mixture stirring at room temperature for 2 h under H₂ atmosphere. The resulting mixture was filtered, the filter cake was washed with MeOH (20 ml), the filtrate was concentrated under reduced pressure to afford 4-{[2-(2-methoxyethoxy) ethanesulfonyl] methyl}aniline (340.00 mg; Crude Product) as an off white solid.

tert-butyl 2-[(4-{[2-(2-methoxyethoxy) ethanesulfonyl] methyl}phenyl) amino]-5H,6H,7H,8H-pyrido[3,4-d]pyrimidine-7-carboxylate: To a stirred mixture of tert-butyl 2-chloro-5H,6H,7H,8H-pyrido[3,4-d]pyrimidine-7-carboxylate (300.00 mg; 1.11 mmol) and 4-{[2-(2-methoxyethoxy)ethane sulfonyl]methyl}aniline (329.14 mg; 1.11 mmol) in Dioxane-1,4 (18.00 ml) were added Xphos Pd G₃ (99.10 mg; 0.11 mmol), Xantphos (130.01 mg; 0.22 mmol) and K₂CO₃ (313.72 mg; 2.22 mmol) at room temperature. After stirring for 5 h at 100° C. under argon atmosphere. The mixture was concentrated under reduced pressure. The residue was purified by silica gel column chromatography, eluted with DCM/MeOH (10:1) to afford tert-butyl 2-[(4-{[2-(2-methoxyethoxy) ethanesulfonyl] methyl}phenyl) amino]-5H,6H,7H,8H-pyrido[3,4-d]pyrimidine-7-carboxylate (300.00 mg 38.76%) as a yellow solid.

N-(4-{[2-(2-methoxyethoxy) ethanesulfonyl] methyl}phenyl)-5H,6H,7H,8H-pyrido[3,4-d]pyrimidin-2-amine: To a stirred solution of tert-butyl 2-[(4-{[2-(2-methoxyethoxy) ethanesulfonyl] methyl}phenyl) amino]-5H,6H,7H,8H-pyrido[3,4-d]pyrimidine-7-carboxylate (290.00 mg; 0.42 mmol) in DCM (5.00 ml) was added TFA (1.00 ml) at room temperature. The resulting mixture was stirred for 1 h at room temperature. The resulting mixture was concentrated under reduced pressure. The mixture was quenched with water/ice (20 ml); basified to pH=14 with NaOH. The resulting mixture was extracted with CH₂Cl₂ (10 ml*3). The combined organic layers were washed with brine (10 ml), dried over anhydrous Na₂SO₄. After filtration, the filtrate was concentrated under reduced pressure. This resulted in N-(4-{[2-(2-methoxyethoxy) ethanesulfonyl] methyl}phenyl)-5H,6H,7H,8H-pyrido[3,4-d]pyrimidin-2-amine (190.00 mg, Crude Product) as a yellow solid.

tert-butyl 7-{2-[(4-{[2-(2-methoxyethoxy) ethanesulfonyl] methyl}phenyl) amino]-5H,6H,7H,8H-pyrido[3,4-d]pyrimidin-7-yl}-8-methyl-1H,2H,3H-pyrido[2,3-b][1,4]oxazine-1-carboxylate: To a stirred mixture of N-(4-{[2-(2-methoxyethoxy) ethanesulfonyl] methyl}phenyl)-5H,6H,7H,8H-pyrido[3,4-d]pyrimidin-2-amine (180.00 mg; 0.32 mmol) and tert-butyl 7-bromo-8-methyl-1H,2H,3H-pyrido[2,3-b][1,4]oxazine-1-carboxylate (126.68 mg; 0.38 mmol) in Dioxane-1,4 (10.00 ml) were added Pd-PEPPSI-IPentCl 2-methylpyridine (o-picoline) (28.36 mg; 0.03 mmol) and Cs₂CO₃ (329.95 mg; 0.96 mmol) at room temperature. After stirring for 2 h at 100° C. under argon atmosphere. The mixture was concentrated under reduced pressure. The residue was purified by silica gel column chromatography, eluted with CH₂Cl₂/MeOH (9:1) to afford tert-butyl 7-{2-[(4-{[2-(2-methoxyethoxy) ethanesulfonyl] methyl}phenyl) amino]-5H,6H,7H,8H-pyrido[3,4-d]pyrimidin-7-yl}-8-methyl-1H,2H,3H-pyrido[2,3-b][1,4]oxazine-1-carboxylate (80.00 mg; 37.85%) as a light yellow solid.

N-(4-{[2-(2-methoxyethoxy) ethanesulfonyl] methyl}phenyl)-7-{8-methyl-1H,2H,3H-pyrido[2,3-b][1,4]oxazin-7-yl}-5H,6H,7H,8H-pyrido[3,4-d]pyrimidin-2-amine: To a stirred solution of tert-butyl 7-{2-[(4-{[2-(2-methoxyethoxy) ethanesulfonyl] methyl}phenyl) amino]-5H,6H,7H,8H-pyrido[3,4-d]pyrimidin-7-yl}-8-methyl-1H,2H,3H-pyrido[2,3-b][1,4]oxazine-1-carboxylate (70.00 mg; 0.11 mmol) in TFE (5.00 ml) at room temperature. The resulting mixture was stirred for 4 h at 120° C. under microwave atmosphere. The mixture was concentrated under reduced pressure. The crude product was purified by Prep-HPLC(Column: XBridge Shield RP18 OBD Column, 30*150 mm, 5 μm; Mobile Phase A: Water (10 mmol NH₄HCO₃+0.05% NH₄OH), Mobile Phase B: ACN; Flow rate: 60 mL/min; Gradient: 34% B to 52% B in 9 min, Wavelength: 254 nm; RT1 (min): 7) to afford N-(4-{[2-(2-methoxyethoxy) ethanesulfonyl] methyl}phenyl)-7-{8-methyl-1H,2H,3H-pyrido[2,3-b][1,4]oxazin-7-yl}-5H,6H,7H,8H-pyrido[3,4-d]pyrimidin-2-amine (40.90 mg; 68.07%;) as a light green solid.

HPLC: 98.03% purity, RT=3.05 min. MS: m/z=555.15 [M+H]⁺. ¹H NMR (300 MHz, DMSO-d6): 9.63 (s, 1H), 8.36 (s, 1H), 7.77 (d, J=8.5 Hz, 2H), 7.28 (d, J=8.4 Hz, 3H), 5.52 (s, 1H), 4.39 (s, 2H), 4.20 (t, J=4.3 Hz, 2H), 3.98 (s, 2H), 3.79 (t, J=5.7 Hz, 2H), 3.64-3.49 (m, 2H), 3.50 (dd, J=5.8, 3.0 Hz, 2H), 3.29 (s, 2H), 3.27 (s, 3H), 3.24 (d, J=5.6 Hz, 2H), 3.12 (d, J=5.3 Hz, 2H), 2.82 (d, J=5.5 Hz, 2H), 2.05 (s, 3H).

### Example 138: Synthesis of Compound 7-{3-fluoro-7-methyl-1H-pyrrolo[3,2-b]pyridin-6-yl}-N-[4-(methanesulfonylmethyl)phenyl]-5H,6H,7H,8H-pyrido[3,4-d]pyrimidin-2-amine

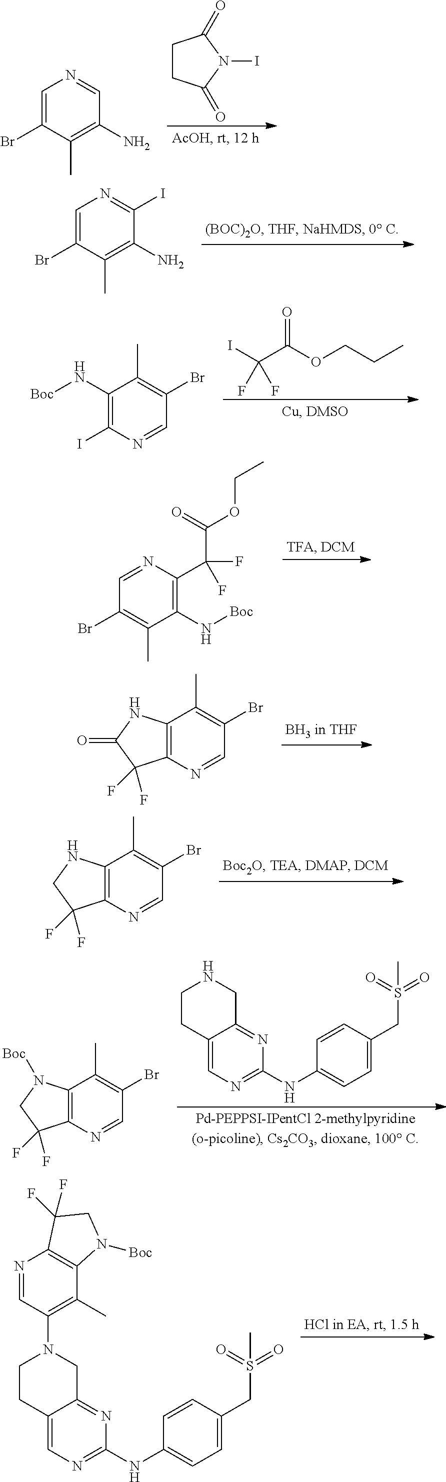

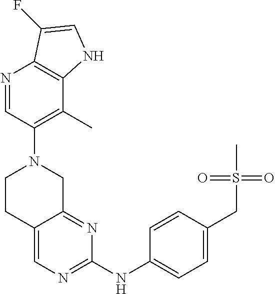 [SMILES: Cc1c(N2CCc3cnc(Nc4ccc(CS(C)(=O)=O)cc4)nc3C2)cnc2c(F)c[nH]c12]

5-bromo-2-iodo-4-methylpyridin-3-amine: To a solution of 5-bromo-4-methylpyridin-3-amine (10.00 g; 50.79 mmol) in AcOH (100.00 ml) was added 1-iodopyrrolidine-2,5-dione (12.03 g; 50.79 mmol). The resulting mixture was stirred for overnight at room temperature under nitrogen atmosphere. The residue was purified by silica gel column chromatography, eluted with PE:EA (3:1) to afford 5-bromo-2-iodo-4-methylpyridin-3-amine (10.00 g; 62.9) as a yellow solid.

tert-butyl N-(5-bromo-2-iodo-4-methylpyridin-3-yl)carbamate: To a stirred solution of 5-bromo-2-iodo-4-methylpyridin-3-amine (10.00 g; 31.92 mmol) in THF (100.00 ml) was added NaHMDS (79.81 ml; 159.62 mmol) at 0° C. under nitrogen atmosphere. The resulting mixture was stirred for 30 mi at 0° C. under nitrogen atmosphere. After 30 min (Boc)₂O (10.79 ml; 47.89 mmol) was added to the above solution at 0° C. under nitrogen atmosphere. Then the resulting mixture was stirred for 2 h at room temperature. The resulting mixture was extracted with EA. The combined organic layers were washed with brine, dried over anhydrous Na₂SO₄. After filtration, the filtrate was concentrated under reduced pressure. The residue was purified by reverse flash chromatography with the following conditions: column, C18 silica gel; mobile phase, ACN in water, 85% to 90% gradient in 30 min; detector, UV 254 nm to afford tert-butyl N-(5-bromo-2-iodo-4-methylpyridin-3-yl)carbamate (9.00 g; 67.7%) as a yellow solid.

ethyl 2-(5-bromo-3-{[(tert-butoxy)carbonyl]amino}-4-methylpyridin-2-yl)-2,2-difluoroacetate: A solution of Cu (0.48 g; 7.21 mmol) and ethyl 2,2-difluoro-2-iodoacetate (3.16 g; 12.01 mmol) in DMSO (50.00 ml) was stirred for 2 h at room temperature under N₂ atmosphere. To the above mixture was added tert-butyl N-(5-bromo-2-iodo-4-methylpyridin-3-yl)carbamate (1.00 g; 2.40 mmol) dropwise at room temperature. The resulting mixture was stirred overnight at 25° C. The resulting mixture was extracted with EA. The combined organic layers were washed with brine, dried over anhydrous Na₂SO₄. After filtration, the filtrate was concentrated under reduced pressure. The residue was purified by reverse flash chromatography with the following conditions: column, C18 silica gel; mobile phase, ACN in water, 50% to 60% gradient in 10 min; detector, UV 254 nm to afford ethyl 2-(5-bromo-3-{[(tert-butoxy)carbonyl]amino}-4-methylpyridin-2-yl)-2,2-difluoroacetate (840.00 mg; 89.4%) as a yellow oil.

6-bromo-3,3-difluoro-7-methyl-1H,2H,3H-pyrrolo[3,2-b]pyridin-2-one: To a stirred solution of ethyl 2-(5-bromo-3-{[(tert-butoxy) carbonyl]amino}-4-methylpyridin-2-yl)-2,2-difluoroacetate (790.00 mg; 1.91 mmol) in DCM (8.00 ml) was added TFA (4.00 ml) at room temperature. The resulting mixture was stirred for 2 days at 40° C. under N₂ atmosphere. The mixture was basified to pH=9 with Saturated sodium bicarbonate solution. The resulting mixture was extracted with EA. The combined organic layers were washed with brine, dried over anhydrous Na₂SO₄. After filtration, the filtrate was concentrated under reduced pressure to afford 6-bromo-3,3-difluoro-7-methyl-1H,2H,3H-pyrrolo[3,2-b]pyridin-2-one (510.00 mg; Crude Product) as a yellow solid.

6-bromo-3,3-difluoro-7-methyl-1H,2H,3H-pyrrolo[3,2-b]pyridine: To a stirred solution of 6-bromo-3,3-difluoro-7-methyl-1H,2H,3H-pyrrolo[3,2-b]pyridin-2-one (500.00 mg; 1.85 mmol) in THF (6.00 ml) was added BH₃ in THF (3.69 ml) dropwise at room temperature. The resulting mixture was stirred for 1 h at room temperature under N₂ atmosphere. The reaction was quenched with HCl in MeOH at 0° C. The resulting mixture was concentrated under reduced pressure. The residue was purified by reverse flash chromatography with the following conditions: column, C18 silica gel; mobile phase, ACN in water, 45% to 50% gradient in 10 min; detector, UV 254 nm to afford 6-bromo-3,3-difluoro-7-methyl-1H,2H,3H-pyrrolo[3,2-b]pyridine (290.00 mg; 60.8%) as a yellow solid.

tert-butyl 6-bromo-3,3-difluoro-7-methyl-1H,2H,3H-pyrrolo[3,2-b]pyridine-1-carboxylate: To a stirred solution of 6-bromo-3,3-difluoro-7-methyl-1H,2H,3H-pyrrolo[3,2-b]pyridine (270.00 mg; 1.05 mmol) in DCM (3.00 ml) were added TEA (0.43 ml), DMAP (27.00 mg; 0.21 mmol) and (Boc)₂O (0.71 ml; 3.14 mmol) at 0° C. The resulting mixture was stirred overnight at room temperature under N₂ atmosphere. The resulting mixture was extracted with EA. The combined organic layers were washed with brine, dried over anhydrous Na₂SO₄. After filtration, the filtrate was concentrated under reduced pressure. The residue was purified by reverse flash chromatography with the following conditions: column, C18 silica gel; mobile phase, ACN in water, 56% to 70% gradient in 20 min; detector, UV 254 nm to afford tert-butyl 6-bromo-3,3-difluoro-7-methyl-1H,2H,3H-pyrrolo[3,2-b]pyridine-1-carboxylate (200.00 mg; 54.6%) as an off-white solid.

tert-butyl 3,3-difluoro-6-(2-{[4-(methanesulfonylmethyl)phenyl]amino}-5H,6H,7H,8H-pyrido[3,4-d]pyrimidin-7-yl)-7-methyl-1H,2H,3H-pyrrolo[3,2-b]pyridine-1-carboxylate: To a stirred mixture of N-[4-(methanesulfonylmethyl)phenyl]-5H,6H,7H,8H-pyrido[3,4-d]pyrimidin-2-amine (160.00 mg; 0.50 mmol) and tert-butyl 6-bromo-3,3-difluoro-7-methyl-1H,2H,3H-pyrrolo[3,2-b]pyridine-1-carboxylate (193.00 mg; 0.55 mmol) in Dioxane-1,4 (2.00 ml) were added Pd-PEPPSI-IPentCl 2-methylpyridine (o-picoline) (44.00 mg; 0.05 mmol) and Cs₂CO₃ (330.00 mg; 1.00 mmol) at room temperature. The resulting mixture was stirred for 1 h at 100° C. under N₂ atmosphere. The resulting mixture was extracted with EA. The combined organic layers were washed with brine, dried over anhydrous Na₂SO₄. After filtration, the filtrate was concentrated under reduced pressure. The residue was purified by silica gel column chromatography, eluted with DCM:MeOH (10:1) to afford tert-butyl 3,3-difluoro-6-(2-{[4-(methanesulfonylmethyl)phenyl]amino}-5H,6H,7H,8H-pyrido[3,4-d]pyrimidin-7-yl)-7-methyl-1H,2H,3H-pyrrolo[3,2-b]pyridine-1-carboxylate (160.00 mg; 47.0%;) as a yellow solid.

7-{3-fluoro-7-methyl-1H-pyrrolo [3,2-b]pyridin-6-yl}-N-[4-(methanesulfonylmethyl)phenyl]-5H,6H,7H,8H-pyrido[3,4-d]pyrimidin-2-amine: To a stirred solution of tert-butyl 3,3-difluoro-6-(2-{[4-(methanesulfonylmethyl)phenyl]amino}-5H,6H,7H,8H-pyrido[3,4-d]pyrimidin-7-yl)-7-methyl-1H,2H,3H-pyrrolo[3,2-b]pyridine-1-carboxylate (50.00 mg; 0.07 mmol) in Ethyl acetate (1.00 ml) was added 4 mol/L HCl (0.30 ml) at 0° C. The resulting mixture was stirred for 40 min at room temperature under nitrogen atmosphere. The resulting mixture was concentrated under vacuum. The crude product was purified by Prep-HPLC with the following conditions (Column: XBridge Shield RP18 OBD Column, 30*150 mm, 5 μm; Mobile Phase A: 10 mmol NH₄HCO₃+0.05% NH4OH, Mobile Phase B: ACN; Flow rate: 60 mL/min; Gradient: 17% to 57% in 9 min; Wavelength: 254 nm; RT1 (min): 7; Number Of Runs: 0) to afford 7-{3-fluoro-7-methyl-1H-pyrrolo[3,2-b]pyridin-6-yl}-N-[4-(methanesulfonylmethyl)phenyl]-5H,6H,7H,8H-pyrido[3,4-d]pyrimidin-2-amine (18.80 mg; 53.0%) as a yellow solid.

HPLC: 96.72% purity, RT=2.70 min. MS: m/z=467.20 [M+H]⁺. ¹H NMR (300 MHz, DMSO-d6) δ 11.01 (d, J=3.0 Hz, 1H), 9.67 (s, 1H), 8.39 (s, 1H), 8.31 (s, 1H), 7.78 (d, J=8.6 Hz, 2H), 7.57 (t, J=2.7 Hz, 1H), 7.28 (d, J=8.5 Hz, 2H), 4.37 (s, 2H), 4.13 (s, 2H), 2.87 (s, 3H), 2.85 (s, 2H), 2.44 (s, 3H)

### Example 139: Synthesis of Compound N-[4-(methanesulfonylmethyl)phenyl]-7-{8′-methyl-1′,2′-dihydrospiro[cyclopropane-1,3′-pyrido[2,3-b][1,4]oxazin]-7′-yl}-5H,6H,7H,8H-pyrido[3,4-d]pyrimidin-2-amine

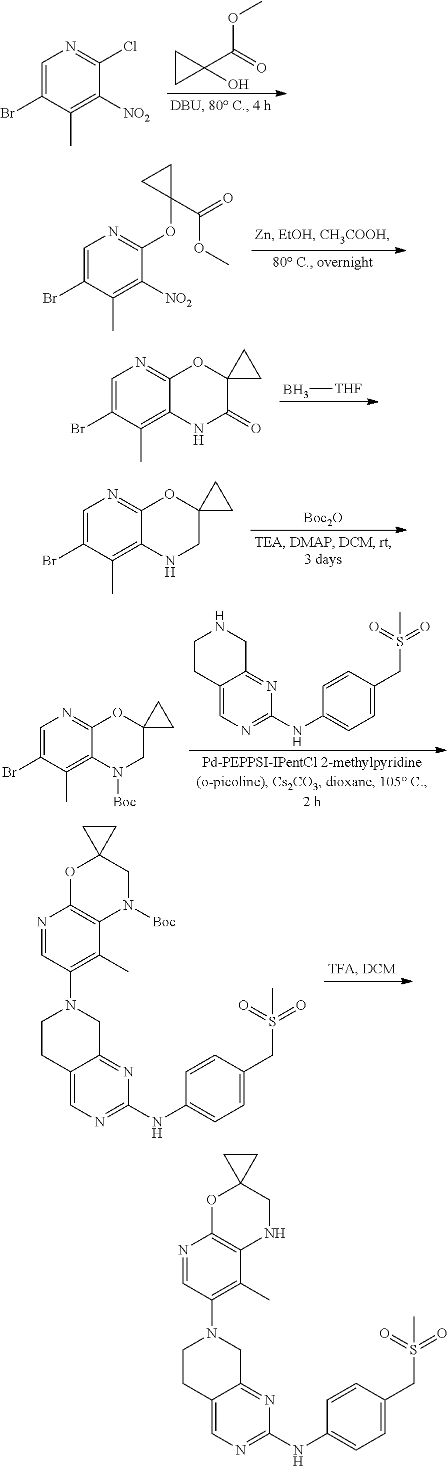

methyl 1-[(5-bromo-4-methyl-3-nitropyridin-2-yl) oxy]cyclopropane-1-carboxylate: To a stirred solution of methyl 1-hydroxycyclopropane-1-carboxylate (7.76 g; 63.47 mmol) and methyl 1-hydroxycyclopropane-1-carboxylate (7.76 g; 63.47 mmol) was added DBU (5.09 g; 31.73 mmol) at 25° C. Then the resulting mixture was stirred for additional 4 h at 80° C. The mixture was concentrated under reduced pressure. The residue was purified by silica gel column chromatography, eluted with PE/EtOAc (6:1) to afford methyl 1-[(5-bromo-4-methyl-3-nitropyridin-2-yl) oxy] cyclopropane-1-carboxylate (900.00 mg; 24.76%) as a yellow solid.

7′-bromo-8′-methyl-1′,2′-dihydrospiro[cyclopropane-1,3′-pyrido[2,3-b][1,4]oxazin]-2′-one: To a stirred mixture of methyl 1-[(5-bromo-4-methyl-3-nitropyridin-2-yl) oxy] cyclopropane-1-carboxylate (900.00 mg; 2.62 mmol) in EtOH (5.00 ml) and AcOH (15.00 ml) was added Zn (991.27 mg; 14.40 mmol). The resulting mixture was stirred for 16 h at 80° C. The resulting mixture was filtered, the filtrate was concentrated under reduced pressure. The residue was purified by silica gel column chromatography, eluted with PE/EtOAc (3:1) to afford 7′-bromo-8′-methyl-1′,2′-dihydrospiro[cyclopropane-1,3′-pyrido[2,3-b][1,4]oxazin]-2′-one (500.00 mg; 69.84%) as an off-white solid.

7′-bromo-8′-methyl-1′,2′-dihydrospiro[cyclopropane-1,3′-pyrido[2,3-b][1,4]oxazine]: To a stirred solution of 7′-bromo-8′-methyl-1′,2′-dihydrospiro[cyclopropane-1,3′-pyrido[2,3-b][1,4]oxazin]-2′-one (500.00 mg; 1.83 mmol) in THF (10.00 ml) was added BH₃ in THF (5.00 ml; 5.00 mmol). The resulting mixture was stirred for 16 h at 40° C. under argon atmosphere. The resulting mixture was extracted with EtOAc and quenched with water/MeOH (10 ml). The combined organic layers were washed with brine (30 ml), dried over anhydrous Na₂SO₄. After filtration, the filtrate was concentrated under reduced pressure. The residue was purified by silica gel column chromatography, eluted with PE/EtOAc (1:1) to afford 7′-bromo-8′-methyl-1′,2′-dihydrospiro[cyclopropane-1,3′-pyrido[2,3-b][1,4]oxazine](240.00 mg; 50.90%) as a glassy solid.

tert-butyl 7′-bromo-8′-methyl-1′,2′-dihydrospiro[cyclopropane-1,3′-pyrido[2,3-b][1,4]oxazine]-1′-carboxylate: To a stirred solution of 7-bromo-8′-methyl-1′,2′-dihydrospiro[cyclopropane-1,3′-pyrido[2,3-b][1,4]oxazine] (240.00 mg; 0.93 mmol) and Di-tert-butyl dicarbonate (641.63 mg; 2.79 mmol) in Dichloromethane (15.00 ml) were added Triethylamine (0.41 ml; 2.79 mmol) and N,N-dimethylpyridin-4-amine (23.94 mg; 0.19 mmol) in portions at room temperature. The resulting mixture was stirred for 16 h at 50° C. The resulting mixture was concentrated under reduced pressure. The residue was purified by silica gel column chromatography, eluted with PE/EtOAc (4:1) to afford tert-butyl 7′-bromo-8′-methyl-1′,2′-dihydrospiro[cyclopropane-1,3′-pyrido[2,3-b][1,4]oxazine]-1′-carboxylate (300.00 mg; 90.44%) as a glassy solid.

tert-butyl 7′-(2-{[4-(methanesulfonylmethyl)phenyl]amino}-5H,6H,7H,8H-pyrido[3,4-d]pyrimidin-7-yl)-8′-methyl-1′,2′-dihydrospiro[cyclopropane-1,3′-pyrido[2,3-b][1,4]oxazine]-1′-carboxylate: To a stirred mixture of N-[4-(methanesulfonylmethyl)phenyl]-5H,6H,7H,8H-pyrido[3,4-d]pyrimidin-2-amine (180.00 mg; 0.54 mmol) and tert-butyl 7′-bromo-8′-methyl-1′,2′-dihydrospiro[cyclopropane-1,3′-pyrido[2,3-b][1,4]oxazine]-1′-carboxylate (230.42 mg; 0.65 mmol) in Dioxane-1,4 (15.00 ml) was added Pd-PEPPSI-IPentCl 2-methylpyridine (o-picoline) (23.83 mg; 0.03 mmol) and Cs₂CO₃ (554.51 mg; 1.62 mmol) at room temperature. After stirring for 16 h at 105° C. under argon atmosphere. The mixture was concentrated under reduced pressure. The residue was purified by silica gel column chromatography, eluted with CH₂Cl₂/MeOH (10:1) to afford tert-butyl 7′-(2-{[4-(methanesulfonylmethyl)phenyl]amino}-5H,6H,7H,8H-pyrido[3,4-d]pyrimidin-7-yl)-8′-methyl-1′,2′-dihydrospiro[cyclopropane-1,3′-pyrido[2,3-b][1,4]oxazine]-1′-carboxylate (240.00 mg; 74.06%;) as a light yellow solid.

N-[4-(methanesulfonylmethyl)phenyl]-7-{8′-methyl-1′,2′-dihydrospiro[cyclopropane-1,3′-pyrido[2,3-b][1,4]oxazin]-7′-yl}-5H,6H,7H,8H-pyrido[3,4-d]pyrimidin-2-amine: To a stirred solution of tert-butyl 7′-(2-{[4-(methanesulfonylmethyl)phenyl]amino}-5H,6H,7H,8H-pyrido[3,4-d]pyrimidin-7-yl)-8′-methyl-1′,2′-dihydrospiro[cyclopropane-1,3′-pyrido[2,3-b][1,4]oxazine]-1′-carboxylate (240.00 mg; 0.40 mmol) in CH₂Cl₂ (8.00 ml) was added TFA (2.00 ml) at room temperature. The resulting mixture was stirred for 4 h at 0° C. temperature. The mixture was concentrated under reduced pressure. The crude product was purified by Prep-HPLC (Column: Xbridge Shield RP18 OBD Column, 30*150 mm, 5 μm; Mobile Phase A: Water (10 mmol/L NH₄HCO₃+0.05% NH₄OH), Mobile Phase B: CAN; Flow rate: 60 mL/min; Gradient: 24% B to 54% B in 9 min, Wavelength: 254 nm; RT1 (min): 7). This resulted in N-[4-(methanesulfonylmethyl)phenyl]-7-{8′-methyl-1′,2′-dihydrospiro[cyclopropane-1,3′-pyrido[2,3-b][1,4]oxazin]-7′-yl}-5H,6H,7H,8H-pyrido[3,4-d]pyrimidin-2-amine (24.5 mg, 12.4%) as a white solid.

HPLC: 99.88% purity, RT=3.14 min. MS: m/z=493.20 [M+H]⁺. ¹H NMR (300 MHz, DMSO-d6): 9.64 (s, 1H), 8.35 (s, 1H), 7.83-7.73 (m, 2H), 7.33-7.24 (m, 3H), 5.65 (t, J=2.8 Hz, 1H), 4.37 (s, 2H), 3.99 (s, 2H), 3.26 (d, J=2.7 Hz, 2H), 3.14 (s, 2H), 2.84 (m, 5H), 2.08 (s, 3H), 0.97-0.81 (m, 2H), 0.78-0.68 (m, 2H).

### Example 140: Synthesis of Compound 6-(2,2-difluoroethyl)-2-[(6-{8-methyl-1H,2H,3H-pyrido[2,3-b][1,4]oxazin-7-yl}-5,6,7,8-tetrahydro-2,6-naphthyridin-3-yl)amino]-4H,5H,6H,7H,8H-pyrazolo[1,5-d][1,4]diazepin-7-one

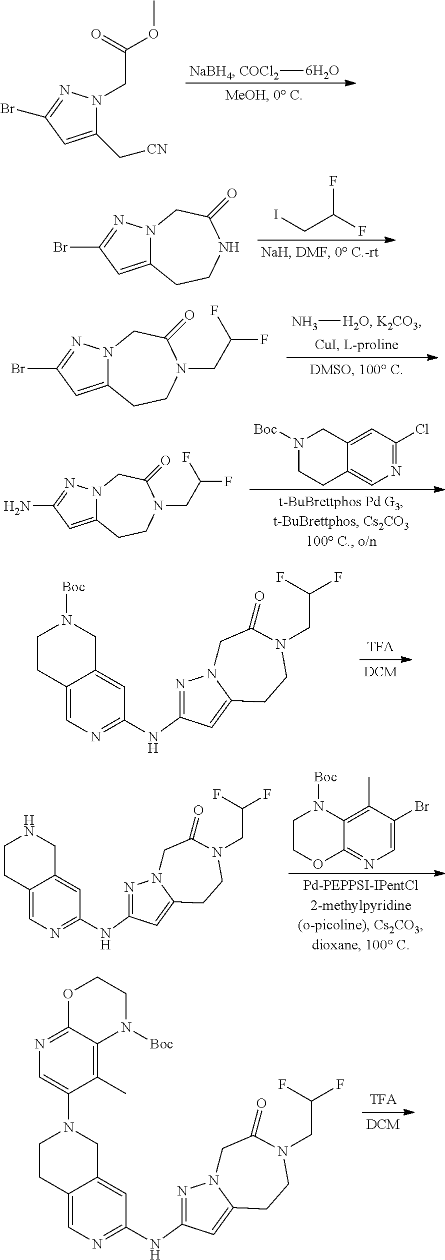

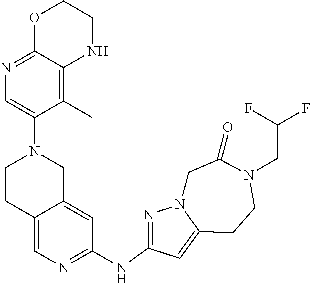 [SMILES: Cc1c(N2CCc3cnc(Nc4cc5n(n4)CC(=O)N(CC(F)F)CC5)cc3C2)cnc2c1NCCO2]

2-bromo-4H,5H,6H,7H,8H-pyrazolo[1,5-d][1,4]diazepin-7-one: To a stirred solution of methyl 2-[3-bromo-5-(cyanomethyl)-1H-pyrazol-1-yl] acetate (4.70 g; 11.04 mmol) and Cobalt chloride hexahydrate (8.70 g; 35.83 mmol) in MeOH (300.00 ml) was added NaBH₄ (3.50 g; 83.27 mmol) in portions at 0° C. under N₂ atmosphere. The resulting mixture was stirred for 2 h at 0° C. under N₂ atmosphere. The resulting mixture was diluted with water, extracted with EtOAc. The combined organic layers were washed with brine, dried over anhydrous Na₂SO₄. The resulting mixture was concentrated under reduced pressure to afford 2-bromo-4H,5H,6H,7H,8H-pyrazolo[1,5-d][1,4]diazepin-7-one (3 g; Crude Product) as a gray solid.

2-bromo-6-(2,2-difluoroethyl)-4H,5H,6H,7H,8H-pyrazolo[1,5-d]diazepin-7-one: To a stirred solution of 2-bromo-4H,5H,6H,7H,8H-pyrazolo[1,5-d][1,4]diazepin-7-one (3.00 g; 11.66 mmol) in DMF (30.00 ml) was added NaH (944.00 mg; 23.60 mmol) in portions at 0° C. under N₂ atmosphere. The resulting mixture was stirred for 30 min at 0° C. under N₂ atmosphere. A solution of 1,1-difluoro-2-iodoethane (12.20 g; 63.56 mmol) was added in portions at 0° C. under N₂ atmosphere. The resulting mixture was stirred for 30 min at room temperature under N₂ atmosphere. The resulting mixture was diluted with water, extracted with EtOAc. The combined organic layers were washed with brine, dried over anhydrous Na₂SO₄. The resulting mixture was concentrated under reduced pressure. The residue was purified by C18 flash column eluted with 30% CAN in water to afford 2-bromo-6-(2,2-difluoroethyl)-4H,5H,6H,7H,8H-pyrazolo[1,5-d][1,4]diazepin-7-one (1.30 g; 33.5%) as an off-white solid.

2-amino-6-(2,2-difluoroethyl)-4H,5H,6H,7H,8H-pyrazolo[1,5-d]diazepin-7-one: To a stirred solution of 2-bromo-6-(2,2-difluoroethyl)-4H,5H,6H,7H,8H-pyrazolo[1,5-d][1,4]diazepin-7-one (1.20 g; 3.60 mmol) and K₂CO₃ (1.20 g; 8.25 mmol) in DMSO (10.00 ml) and NH₄OH (2.00 ml) were added CuI (156.00 mg; 0.78 mmol) and L-Proline (189.00 mg; 1.56 mmol) in portions at room temperature under N₂ atmosphere. The resulting mixture was stirred for overnight at 100° C. under N₂ atmosphere in a sealed tube. The residue was purified by silica gel column, eluted with 20% MeOH in CH₂Cl₂ to afford crude product. The crude product was repurified by C18 flash column eluted with 60% CAN in Water to afford 2-amino-6-(2,2-difluoroethyl)-4H,5H,6H,7H,8H-pyrazolo[1,5-d][1,4]diazepin-7-one (430.00 mg; 48.9%;) as a yellow solid.

Tert-butyl 7-{[6-(2,2-difluoroethyl)-7-oxo-4H,5H,6H,7H,8H-pyrazolo[1,5-d][1,4]diazepin-2-yl]amino}-1,2,3,4-tetrahydro-2,6-naphthyridine-2-carboxylate: To a stirred solution of 2-amino-6-(2,2-difluoroethyl)-4H,5H,6H,7H,8H-pyrazolo[1,5-d][1,4]diazepin-7-one (410.00 mg; 1.68 mmol) and tert-butyl 7-chloro-1,2,3,4-tetrahydro-2,6-naphthyridine-2-carboxylate (526.00 mg; 1.92 mmol) in 1,4-dioxane (10.00 ml) were added t-BuBrettPhos Pd G₃ (153.00 mg; 0.17 mmol) t-BuBrettPhos (173.00 mg; 0.34 mmol) and Cs₂CO₃ (1.80 g; 5.25 mmol) in portions at room temperature under N₂ atmosphere. The resulting mixture was stirred for overnight at 100° C. under N₂ atmosphere. The resulting mixture was diluted with water, extracted with EtOAc. The combined organic layers were washed with brine, dried over anhydrous Na₂SO₄. The resulting mixture was concentrated under reduced pressure. The residue was purified by silica gel column, eluted with 100% EtOAc to afford tert-butyl 7-{[6-(2,2-difluoroethyl)-7-oxo-4H,5H,6H,7H,8H-pyrazolo[1,5-d][1,4]diazepin-2-yl]amino}-1,2,3,4-tetrahydro-2,6-naphthyridine-2-carboxylate (410.00 mg; 50.7%) as an off-white solid.

6-(2,2-difluoroethyl)-2-[(5,6,7,8-tetrahydro-2,6-naphthyridin-3-yl) amino]-4H,5H,6H,7H,8H-pyrazolo[1,5-d][1,4]diazepin-7-one: To a stirred solution of tert-butyl 7-{[6-(2,2-difluoroethyl)-7-oxo-4H,5H,6H,7H,8H-pyrazolo[1,5-d][1,4]diazepin-2-yl]amino}-1,2,3,4-tetrahydro-2,6-naphthyridine-2-carboxylate (400.00 mg; 0.83 mmol) in CH₂Cl₂ (3.00 ml) was added TFA (1.00 ml) at 0° C. under N₂ atmosphere. The resulting mixture was stirred for 30 min at room temperature under N₂ atmosphere. The resulting mixture was concentrated under reduced pressure. The residue was purified by C18 flash column eluted with 10% CAN in water to afford 6-(2,2-difluoroethyl)-2-[(5,6,7,8-tetrahydro-2,6-naphthyridin-3-yl) amino]-4H,5H,6H,7H,8H-pyrazolo[1,5-d][1,4]diazepin-7-one (150.00 mg; 49.4%) as an off-white solid.

Tert-butyl 7-(7-{[6-(2,2-difluoroethyl)-7-oxo-4H,5H,6H,7H,8H-pyrazolo[1,5-d][1,4]diazepin-2-yl]amino}-1,2,3,4-tetrahydro-2,6-naphthyridin-2-yl)-8-methyl-1H,2H,3H-pyrido[2,3-b][1,4]oxazine-1-carboxylate: To a stirred solution of 6-(2,2-difluoroethyl)-2-[(5,6,7,8-tetrahydro-2,6-naphthyridin-3-yl) amino]-4H,5H,6H,7H,8H-pyrazolo[1,5-d][1,4]diazepin-7-one (130.00 mg; 0.36 mmol) and tert-butyl 7-bromo-8-methyl-1H,2H,3H-pyrido[2,3-b][1,4]oxazine-1-carboxylate (236.00 mg; 0.72 mmol) in 1,4-dioxane (5.00 ml) were added Pd-PEPPSI-IpentCl 2-methylpyridine (o-picoline) (31.00 mg; 0.04 mmol) and Cs₂CO₃ (352.00 mg; 1.03 mmol) in portions at room temperature under N₂ atmosphere. The resulting mixture was stirred for overnight at 100° C. under N₂ atmosphere. The resulting mixture was diluted with water, extracted with EtOAc. The combined organic layers were washed with brine, dried over anhydrous Na₂SO₄. The resulting mixture was concentrated under reduced pressure. The residue was purified by silica gel column, eluted with 10% MeOH in CH₂Cl₂ to afford tert-butyl 7-(7-{[6-(2,2-difluoroethyl)-7-oxo-4H,5H,6H,7H,8H-pyrazolo[1,5-d][1,4]diazepin-2-yl]amino}-1,2,3,4-tetrahydro-2,6-naphthyridin-2-yl)-8-methyl-1H,2H,3H-pyrido[2,3-b][1,4]oxazine-1-carboxylate (180.00 mg; 75.5%) yellow solid.

6-(2,2-difluoroethyl)-2-[(6-{8-methyl-1H,2H,3H-pyrido[2,3-b][1,4]oxazin-7-yl}-5,6,7,8-tetrahydro-2,6-naphthyridin-3-yl) amino]-4H,5H,6H,7H,8H-pyrazolo[1,5-d][1,4]diazepin-7-one: To a stirred solution of tert-butyl 7-(7-{[6-(2,2-difluoroethyl)-7-oxo-4H,5H,6H,7H,8H-pyrazolo[1,5-d][1,4]diazepin-2-yl]amino}-1,2,3,4-tetrahydro-2,6-naphthyridin-2-yl)-8-methyl-1H,2H,3H-pyrido[2,3-b][1,4]oxazine-1-carboxylate (170.00 mg; 0.25 mmol) in CH₂Cl₂ (3.00 ml) was added TFA (1.00 ml) at 0° C. under N₂ atmosphere. The resulting mixture was stirred for 30 min at room temperature under N₂ atmosphere. The resulting mixture was concentrated under reduced pressure. The residue was purified by Prep-HPLC with following condition: Column: Xbridge Prep OBD C18 Column, 30*150 mm, 5 μm; Mobile Phase A: Water (10 mmol/L NH₄HCO₃), Mobile Phase B: CAN; Flow rate: 60 mL/min; Gradient: 25% B to 55% B in 8 min, 55% B; Wavelength: 254 nm; RT1 (min): 7 to afford 6-(2,2-difluoroethyl)-2-[(6-{8-methyl-1H,2H,3H-pyrido[2,3-b]oxazin-7-yl}-5,6,7,8-tetrahydro-2,6-naphthyridin-3-yl) amino]-4H,5H,6H,7H,8H-pyrazolo[1,5-d][1,4]diazepin-7-one (72.10 mg; 55.2%) as an off-white solid.

HPLC: 99.21% purity, RT=2.31 min. MS: m/z=511.25 [M+H]⁺. ¹H NMR (300 MHz, DMSO-d6) δ 9.00 (s, 1H), 7.94 (s, 1H), 7.26 (s, 1H), 7.01 (s, 1H), 6.60-5.95 (m, 2H), 5.50 (s, 1H), 4.99 (s, 2H), 4.18 (s, 2H), 4.00-3.75 (m, 6H), 3.29 (s, 2H), 3.06 (s, 4H), 2.79 (s, 2H), 2.03 (s, 3H).

### Example 141: Synthesis of Compound 6-cyclopropyl-2-[(6-{8-methyl-1H,2H,3H-pyrido[2,3-b][1,4]oxazin-7-yl}-5,6,7,8-tetrahydro-2,6-naphthyridin-3-yl)amino]-4H,5H,6H,7H,8H-pyrazolo[1,5-d][1,4]diazepin-7-one

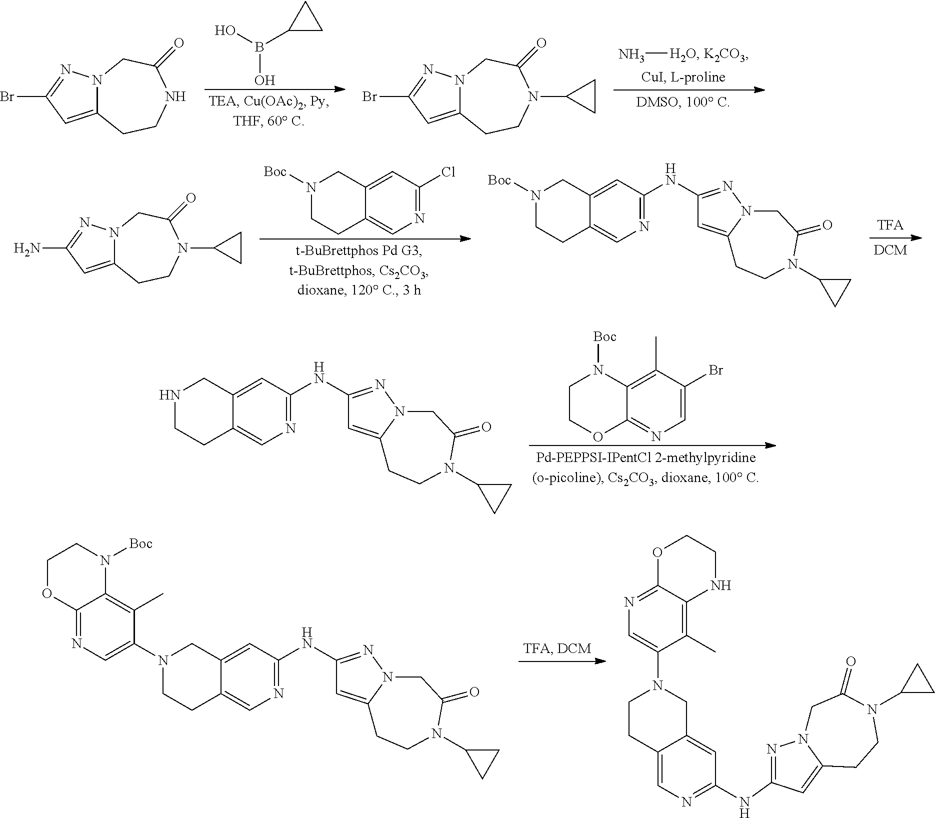

2-bromo-6-cyclopropyl-4H,5H,6H,7H,8H-pyrazolo[1,5-d][1,4]diazepin-7-one: To a stirred solution of 2-bromo-4H,5H,6H,7H,8H-pyrazolo[1,5-d][1,4]diazepin-7-one (1.60 g; 6.86 mmol) and cyclopropylboronic acid (1.80 g; 19.91 mmol) in THF (50.00 ml) were added Et₃N (2.10 g; 18.68 mmol), Cupric acetate monohydrate (1.30 g; 6.19 mmol) and pyridine (828.00 mg; 9.42 mmol) in portions at room temperature under O₂ atmosphere. The resulting mixture was stirred for overnight at 60° C. under O₂ atmosphere. The resulting mixture was diluted with water, extracted with EtOAc. The combined organic layers were washed with brine, dried over anhydrous Na₂SO₄. The resulting mixture was concentrated under reduced pressure. The residue was purified by C18 flash column eluted with 30% CAN in Water to afford 2-bromo-6-cyclopropyl-4H,5H,6H,7H,8H-pyrazolo[1,5-d][1,4]diazepin-7-one (1.00 g; 39.7%) as a brown oil.

2-amino-6-cyclopropyl-4H,5H,6H,7H,8H-pyrazolo[1,5-d][1,4]diazepin-7-one: To a stirred solution of 2-bromo-6-cyclopropyl-4H,5H,6H,7H,8H-pyrazolo[1,5-d][1,4]diazepin-7-one (900.00 mg; 2.45 mmol) and K₂CO₃ (924.00 mg; 6.35 mmol) in DMSO (5.00 ml) and NH₄OH (1.00 ml) were added CuI (128.00 mg; 0.64 mmol) and L-Proline (154.00 mg; 1.27 mmol) in portions at room temperature under N₂ atmosphere. The resulting mixture was stirred for overnight at 100° C. under N₂ atmosphere in a sealed tube. The residue was purified by C18 flash column eluted with 20% ACN in Water to afford 2-amino-6-cyclopropyl-4H,5H,6H,7H,8H-pyrazolo[1,5-d][1,4]diazepin-7-one (290.00 mg; 54.6%) as a yellow solid.

tert-butyl 7-({6-cyclopropyl-7-oxo-4H,5H,6H,7H,8H-pyrazolo[1,5-d][1,4]diazepin-2-yl}amino)-1,2,3,4-tetrahydro-2,6-naphthyridine-2-carboxylate: To a stirred solution of 2-amino-6-cyclopropyl-4H,5H,6H,7H,8H-pyrazolo[1,5-d][1,4]diazepin-7-one (280.00 mg; 1.29 mmol) and tert-butyl 7-chloro-1,2,3,4-tetrahydro-2,6-naphthyridine-2-carboxylate (438.00 mg; 1.60 mmol) in 1,4-dioxane (5.00 ml) were added t-BuBrettPhos Pd G₃ (117.00 mg; 0.13 mmol), t-BuBrettPhos (132.00 mg; 0.26 mmol) and Cs₂CO₃ (1.33 g; 3.88 mmol) in portions at room temperature. The resulting mixture was stirred for overnight at 120° C. under N₂ atmosphere. The resulting mixture was diluted with water, extracted with EtOAc. The combined organic layers were washed with brine, dried over anhydrous Na₂SO₄. After filtration, the resulting mixture was concentrated under reduced pressure. The residue was purified by silica gel column, eluted with 10% MeOH in CH₂Cl₂ to afford tert-butyl 7-({6-cyclopropyl-7-oxo-4H,5H,6H,7H,8H-pyrazolo[1,5-d][1,4]diazepin-2-yl}amino)-1,2,3,4-tetrahydro-2,6-naphthyridine-2-carboxylate (270.00 mg; 38.7%) as a yellow solid.

6-cyclopropyl-2-[(5,6,7,8-tetrahydro-2,6-naphthyridin-3-yl) amino]-4H,5H,6H,7H,8H-pyrazolo[1,5-d][1,4]diazepin-7-one: To a stirred solution of tert-butyl 7-({6-cyclopropyl-7-oxo-4H,5H,6H,7H,8H-pyrazolo[1,5-d][1,4]diazepin-2-yl}amino)-1,2,3,4-tetrahydro-2,6-naphthyridine-2-carboxylate (260.00 mg; 0.48 mmol) in CH₂Cl₂ (3.00 ml) was added TFA (1.00 ml) at 0° C. under N₂ atmosphere. The resulting mixture was stirred for 2 h at room temperature under N₂ atmosphere. The resulting mixture was concentrated under reduced pressure. The residue was purified by C18 flash column eluted with 300% ACN in water to afford 6-cyclopropyl-2-[(5,6,7,8-tetrahydro-2,6-naphthyridin-3-yl) amino]-4H,5H,6H,7H,8H-pyrazolo[1,5-d][1,4]diazepin-7-one (180.00 mg; 97.5%) as a white solid.

tert-butyl 7-[7-({6-cyclopropyl-7-oxo-4H,5H,6H,7H,8H-pyrazolo[1,5-d][1,4]diazepin-2-yl}amino)-1,2,3,4-tetrahydro-2,6-naphthyridin-2-yl]-8-methyl-1H,2H,3H-pyrido[2,3-b][1,4]oxazine-1-carboxylate: To a stirred solution of 6-cyclopropyl-2-[(5,6,7,8-tetrahydro-2,6-naphthyridin-3-yl) amino]-4H,5H,6H,7H,8H-pyrazolo[1,5-d][1,4]diazepin-7-one (170.00 mg; 0.44 mmol) and tert-butyl 7-bromo-8-methyl-1H,2H,3H-pyrido[2,3-b][1,4]oxazine-1-carboxylate (330.00 mg; 1.00 mmol) in 1,4-dioxane (5.00 ml) were added Pd-PEPPSI-IPentCl 2-methylpyridine (o-picoline) (43.00 mg; 0.05 mmol) and Cs₂CO₃ (495.00 mg; 1.44 mmol) in portions at room temperature. The resulting mixture was stirred for overnight at 100° C. under N₂ atmosphere. The resulting mixture was diluted with water, extracted with EtOAc. The combined organic layers were washed with brine, dried over anhydrous Na₂SO₄. After filtration, the resulting mixture was concentrated under reduced pressure. The residue was purified by silica gel column, eluted with 15% MeOH in CH₂Cl₂ to afford tert-butyl 7-[7-({6-cyclopropyl-7-oxo-4H,5H,6H,7H,8H-pyrazolo[1,5-d][1,4]diazepin-2-yl}amino)-1,2,3,4-tetrahydro-2,6-naphthyridin-2-yl]-8-methyl-1H,2H,3H-pyrido[2,3-b][1,4]oxazine-1-carboxylate (150.00 mg; 50.2%) as a yellow solid.

6-cyclopropyl-2-[(6-{8-methyl-1H,2H,3H-pyrido[2,3-b][1,4]oxazin-7-yl}-5,6,7,8-tetrahydro-2,6-naphthyridin-3-yl) amino]-4H,5H,6H,7H,8H-pyrazolo[1,5-d][1,4]diazepin-7-one: To a stirred solution of tert-butyl 7-[7-({6-cyclopropyl-7-oxo-4H,5H,6H,7H,8H-pyrazolo[1,5-d][1,4]diazepin-2-yl}amino)-1,2,3,4-tetrahydro-2,6-naphthyridin-2-yl]-8-methyl-1H,2H,3H-pyrido[2,3-b][1,4]oxazine-1-carboxylate (140.00 mg; 0.21 mmol) in CH₂Cl₂ (3.00 ml) was added TFA (1.00 ml) at 0° C. under N₂ atmosphere. The resulting mixture was stirred for 30 min at room temperature under N₂ atmosphere. The resulting mixture was concentrated under reduced pressure. The residue was purified by Prep-HPLC with following condition: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 25% B to 55% B in 8 min, Wavelength: 254 nm; RT1 (min): 7; to afford 6-cyclopropyl-2-[(6-{8-methyl-1H,2H,3H-pyrido[2,3-b][1,4]oxazin-7-yl}-5,6,7,8-tetrahydro-2,6-naphthyridin-3-yl) amino]-4H,5H,6H,7H,8H-pyrazolo[1,5-d][1,4]diazepin-7-one (34.70 mg; 32.7%) as an off-white solid.

HPLC: 95.09% purity, RT=2.17 min. MS: m/z=487.15 [M+H]⁺. ¹H NMR (300 MHz, DMSO-d6) δ 10.71 (s, 1H), 8.06 (s, 1H), 7.28 (s, 1H), 7.08 (s, 1H), 6.03 (s, 1H), 5.08 (s, 2H), 4.29-4.10 (m, 4H), 3.82 (d, J=6.2 Hz, 2H), 3.32 (t, J=4.3 Hz, 2H), 3.16-2.97 (m, 4H), 2.95-2.75 (m, 3H), 2.05 (s, 3H), 0.70 (m, 4H).

### Example 142: Synthesis of Compound 7-(2-{[4-(methanesulfonylmethyl)phenyl]amino}-5H,6H,7H,8H-pyrido[3,4-d]pyrimidin-7-yl)-8-methyl-1,2,3,4-tetrahydro-1,5-naphthyridin-4-one

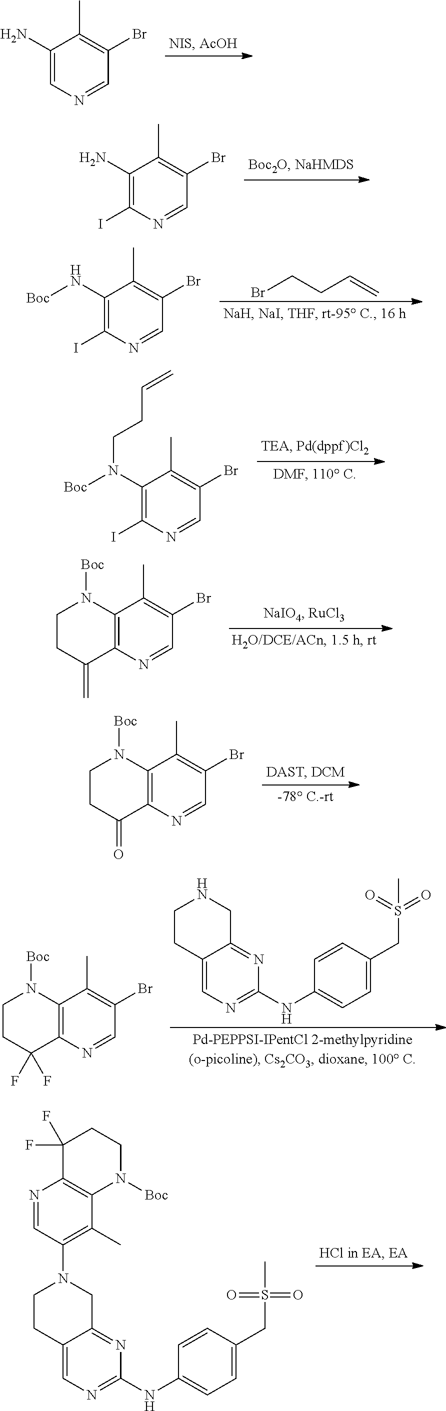

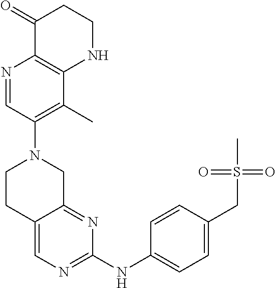 [SMILES: Cc1c(N2CCc3cnc(Nc4ccc(CS(C)(=O)=O)cc4)nc3C2)cnc2c1NCCC2=O]

5-bromo-2-iodo-4-methylpyridin-3-amine: To a solution of 5-bromo-4-methylpyridin-3-amine (18.00 g; 91.43 mmol) in AcOH (200.00 ml) were added 1-iodopyrrolidine-2,5-dione (21.65 g; 91.43 mmol). The resulting mixture was stirred for overnight at room temperature under nitrogen atmosphere. The resulting mixture was concentrated under vacuum. The residue was purified by silica gel column chromatography, eluted with PE/EtOAc (4:1) to afford 5-bromo-2-iodo-4-methylpyridin-3-amine (18.00 g; 46.5%) as a yellow solid.

tert-butyl N-(5-bromo-2-iodo-4-methylpyridin-3-yl)carbamate: To a stirred solution of 5-bromo-2-iodo-4-methylpyridin-3-amine (18.00 g; 42.55 mmol) in THF (200.00 ml) was added NaHMDS (106.37 ml; 212.74 mmol) at 0° C. under nitrogen atmosphere. The resulting mixture was stirred for 30 min at 0° C. under nitrogen atmosphere. After 30 min (Boc)₂O (10.54 ml; 46.80 mmol) was added to the above solution 0° C. under nitrogen atmosphere. Then the resulting mixture was stirred for additional 2 h at room temperature. The resulting mixture was concentrated under reduced pressure. The residue was purified by silica gel column chromatography, eluted with PE/EA (5:1) to afford tert-butyl N-(5-bromo-2-iodo-4-methylpyridin-3-yl)carbamate (16.00 g; 71.8%) as a yellow solid.

tert-butyl N-(5-bromo-2-iodo-4-methylpyridin-3-yl)-N-(but-3-en-1-yl)carbamate: To a stirred solution of tert-butyl N-(5-bromo-2-iodo-4-methylpyridin-3-yl)carbamate (4.00 g; 7.63 mmol) in THF (50.00 ml) was added NaH (698.00 mg; 17.45 mmol) in portions at room temperature under N₂ atmosphere. The resulting mixture was stirred for 30 min at room temperature under N₂ atmosphere. A solution of 4-bromobut-1-ene (5.30 g; 37.30 mmol) was then added and heating at 95° C. for overnight. The resulting mixture was diluted with water, extracted with EtOAc. The combined organic layers were washed with brine, dried over anhydrous Na₂SO₄. The resulting mixture was concentrated under reduced pressure. The residue was purified by silica gel column, eluted with 15% EtOAc in PE to afford tert-butyl N-(5-bromo-2-iodo-4-methylpyridin-3-yl)-N-(but-3-en-1-yl)carbamate (3.00 g; 73.8%) as a yellow oil.

tert-butyl 7-bromo-8-methyl-4-methylidene-1,2,3,4-tetrahydro-1,5-naphthyridine-1-carboxylate: To a stirred solution of tert-butyl N-(5-bromo-2-iodo-4-methylpyridin-3-yl)-N-(but-3-en-1-yl)carbamate (2.90 g; 5.45 mmol) in DMF (20.00 ml) were added Et₃N (1.30 g; 11.56 mmol) and 1,1′-Bis(diphenylphosphino) ferrocene-palladium(II)dichloride dichloromethane complex (255.00 mg; 0.30 mmol) in portions at room temperature under N₂ atmosphere. The resulting mixture was stirred for 2 h at 110° C. under N₂ atmosphere. The resulting mixture was diluted with water, extracted with EtOAc. The combined organic layers were washed with brine, dried over anhydrous Na₂SO₄. The resulting mixture was concentrated under reduced pressure. The residue was purified by silica gel column, eluted with 15% EtOAc in PE to afford tert-butyl 7-bromo-8-methyl-4-methylidene-1,2,3,4-tetrahydro-1,5-naphthyridine-1-carboxylate (1.30 g; 70.1%) as a yellow oil.

tert-butyl 7-bromo-8-methyl-4-oxo-1,2,3,4-tetrahydro-1,5-naphthyridine-1-carboxylate: To a stirred solution of tert-butyl 7-bromo-8-methyl-4-methylidene-1,2,3,4-tetrahydro-1,5-naphthyridine-1-carboxylate (1.20 g; 3.53 mmol) and NaIO₄ (1.90 g; 8.44 mmol) in water (10.00 ml); 1,2-Dichloroethane (10.00 ml) and MeCN (10.00 ml) was added RuCl₃ (240.00 mg; 1.10 mmol) in portions at room temperature under N₂ atmosphere. The resulting mixture was stirred for 3 h at room temperature under N₂ atmosphere. The resulting mixture was diluted with water, extracted with EtOAc. The combined organic layers were washed with brine, dried over anhydrous Na₂SO₄. The resulting mixture was concentrated under reduced pressure. The residue was purified by silica gel column, eluted with 30% EtOAc in PE to afford tert-butyl 7-bromo-8-methyl-4-oxo-1,2,3,4-tetrahydro-1,5-naphthyridine-1-carboxylate (410.00 mg; 33.6%) as a green solid.

tert-butyl 7-bromo-4,4-difluoro-8-methyl-1,2,3,4-tetrahydro-1,5-naphthyridine-1-carboxylate: To a stirred solution of tert-butyl 7-bromo-8-methyl-4-oxo-1,2,3,4-tetrahydro-1,5-naphthyridine-1-carboxylate (500.00 mg; 1.47 mmol) in dichloromethane (5.00 ml) was added DAST (25.00 ml) dropwised at −78° C. under N₂ atmosphere. The resulting mixture was stirred for overnight at room temperature under N₂ atmosphere. The reaction was quenched with NaHCO₃(aq) at 0° C. and extracted with EtOAc. The combined organic layers were washed with brine, dried over anhydrous Na₂SO₄. The resulting mixture was concentrated under reduced pressure. The residue was purified by silica gel column, eluted with 15% EtOAc in PE to afford tert-butyl 7-bromo-4,4-difluoro-8-methyl-1,2,3,4-tetrahydro-1,5-naphthyridine-1-carboxylate (270.00 mg; 50.7%) as an off-white solid.

tert-butyl 4,4-difluoro-7-(2-{[4-(methanesulfonylmethyl)phenyl]amino}-5H,6H,7H,8H-pyrido[3,4-d]pyrimidin-7-yl)-8-methyl-1,2,3,4-tetrahydro-1,5-naphthyridine-1-carboxylate: To a stirred solution of tert-butyl 7-bromo-4,4-difluoro-8-methyl-1,2,3,4-tetrahydro-1,5-naphthyridine-1-carboxylate (250.00 mg; 0.69 mmol) and N-[4-(methanesulfonylmethyl)phenyl]-5H,6H,7H,8H-pyrido[3,4-d]pyrimidin-2-amine (242.00 mg; 0.76 mmol) in 1,4-dioxane (5.00 ml) were added Pd-PEPPSI-IPentCl 2-methylpyridine (o-picoline) (59.00 mg; 0.07 mmol) and Cs₂CO₃ (676.00 mg; 1.97 mmol) in portions at room temperature under N₂ atmosphere. The resulting mixture was stirred for 2 h at 100° C. under N₂ atmosphere. The resulting mixture was diluted with water and extracted with EtOAc. The combined organic layers were washed with brine, dried over anhydrous Na₂SO₄. The resulting mixture was concentrated under reduced pressure. The residue was purified by silica gel column, eluted with 85% EtOAc in PE to afford tert-butyl 4,4-difluoro-7-(2-{[4-(methanesulfonylmethyl) phenyl]amino}-5H,6H,7H,8H-pyrido[3,4-d]pyrimidin-7-yl)-8-methyl-1,2,3,4-tetrahydro-1,5-naphthyridine-1-carboxylate (310.00 mg; 75.0%) as a yellow solid.

7-(2-{[4-(methanesulfonylmethyl)phenyl]amino}-5H,6H,7H,8H-pyrido[3,4-d]pyrimidin-7-yl)-8-methyl-1,2,3,4-tetrahydro-1,5-naphthyridin-4-one: To a stirred solution of tert-butyl 4,4-difluoro-7-(2-{[4-(methanesulfonylmethyl) phenyl]amino}-5H,6H,7H,8H-pyrido[3,4-d]pyrimidin-7-yl)-8-methyl-1,2,3,4-tetrahydro-1,5-naphthyridine-1-carboxylate (190.00 mg; 0.32 mmol) in Ethyl acetate (2.00 ml) was added 4N HCl in EA (2.00 ml; 8.00 mmol) dropwise at 0° C. under N₂ atmosphere. The resulting mixture was concentrated under reduced pressure. The residue was purified by Prep-HPLC with following condition: Column: XBridge Prep OBD C18 Column, 30*150 mm, 5 μm; Mobile Phase A: Water (10 mmol/L NH₄HCO₃+0.05% NH₄OH), Mobile Phase B: ACN; Flow rate: 60 mL/min; Gradient: 19% B to 47% B in 9 min, Wavelength: 254 nm; RT1 (min): 7; Number Of Runs: 2 to afford 7-(2-{[4-(methanesulfonylmethyl)phenyl]amino}-5H,6H,7H,8H-pyrido[3,4-d]pyrimidin-7-yl)-8-methyl-1,2,3,4-tetrahydro-1,5-naphthyridin-4-one (23.00 mg; 54.5%) as a yellow solid.

HPLC: 95.04% purity, RT=2.56 min. MS: m/z=479.10 [M+H]⁺. ¹H NMR (400 MHz, DMSO-d6) δ 9.67 (s, 1H), 8.39 (s, 1H), 7.87 (s, 1H), 7.78 (d, J=8.5 Hz, 2H), 7.29 (d, J=8.6 Hz, 2H), 6.98 (s, 1H), 4.38 (s, 2H), 4.27 (s, 2H), 3.56 (t, J=7.2 Hz, 2H), 3.35 (d, J=6.0 Hz, 2H), 2.87 (s, 5H), 2.67 (t, J=7.1 Hz, 2H), 2.19 (s, 3H).

### Example 143: Synthesis of Compound 7-(2-{[4-(methanesulfonylmethyl)phenyl]amino}-5H,6H,7H,8H-pyrido[3,4-d]pyrimidin-7-yl)-8-methyl-1,2,3,4-tetrahydro-1,5-naphthyridin-4-ol

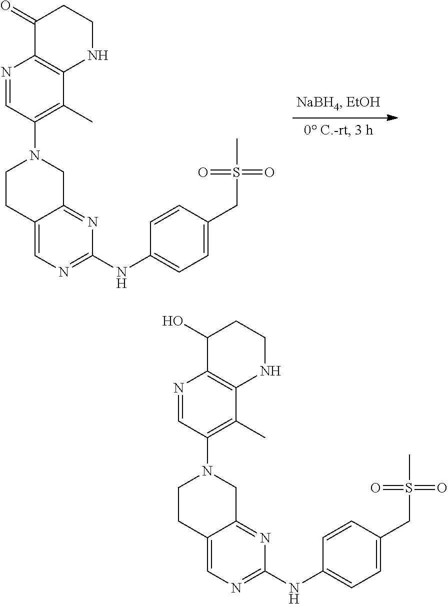 [SMILES: Cc1c(N2CCc3cnc(Nc4ccc(CS(C)(=O)=O)cc4)nc3C2)cnc2c1NCCC2=O ; Cc1c(N2CCc3cnc(Nc4ccc(CS(C)(=O)=O)cc4)nc3C2)cnc2c1NCCC2O]

7-(2-{[4-(methanesulfonylmethyl)phenyl]amino}-5H,6H,7H,8H-pyrido[3,4-d]pyrimidin-7-yl)-8-methyl-1,2,3,4-tetrahydro-1,5-naphthyridin-4-ol: To a stirred solution of 7-(2-{[4-(methanesulfonylmethyl)phenyl]amino}-5H,6H,7H,8H-pyrido[3,4-d]pyrimidin-7-yl)-8-methyl-1,2,3,4-tetrahydro-1,5-naphthyridin-4-one (80.00 mg; 0.17 mmol) in EtOH (3.00 ml) was added NaBH₄ (24.00 mg; 0.57 mmol) in portions at 0° C. under N₂ atmosphere. The resulting mixture was stirred for 1 h at room temperature under N₂ atmosphere. The resulting mixture was diluted with water, extracted with EtOAc. The combined organic layers were washed with brine, dried over anhydrous Na₂SO₄. The resulting mixture was concentrated under reduced pressure. The residue was purified by Prep-HPLC with following condition: Column: XBridge Shield RP18 OBD Column, 30*150 mm, 5 μm; Mobile Phase A: Water (10 mmol/L NH₄HCO₃+0.05% NH₄OH), Mobile Phase B: ACN; Flow rate: 60 mL/min; Gradient: 18% B to 48% B in 9 min, Wavelength: 254 nm; RT1 (min): 7; Number Of Runs: 2 to afford 7-(2-{[4-(methanesulfonylmethyl)phenyl]amino}-5H,6H,7H,8H-pyrido[3,4-d]pyrimidin-7-yl)-8-methyl-1,2,3,4-tetrahydro-1,5-naphthyridin-4-ol (47.40 mg; 58.0%) as a white solid.

HPLC: 98.35% purity, RT=2.66 min. MS: m/z=481.10 [M+H]⁺. ¹H NMR (400 MHz, DMSO-d6) δ 9.66 (s, 1H), 8.36 (s, 1H), 7.78 (d, J=8.3 Hz, 2H), 7.66 (s, 1H), 7.29 (d, J=8.3 Hz, 2H), 5.48 (s, 1H), 5.02 (s, 1H), 4.54 (s, 1H), 4.38 (s, 2H), 4.04 (s, 2H), 3.19 (t, J=6.0 Hz, 4H), 2.87 (s, 3H), 2.82 (d, J=5.7 Hz, 2H), 2.02 (s, 3H), 1.94-1.66 (m, 2H).

### Example 144: Synthesis of Compound 6-{8-methyl-1H,2H,3H-pyrido[2,3-b][1,4]oxazin-7-yl}-N-{4-[(1H-1,2,4-triazol-1-yl) methyl]phenyl}-5,6,7,8-tetrahydro-2,6-naphthyridin-3-amine

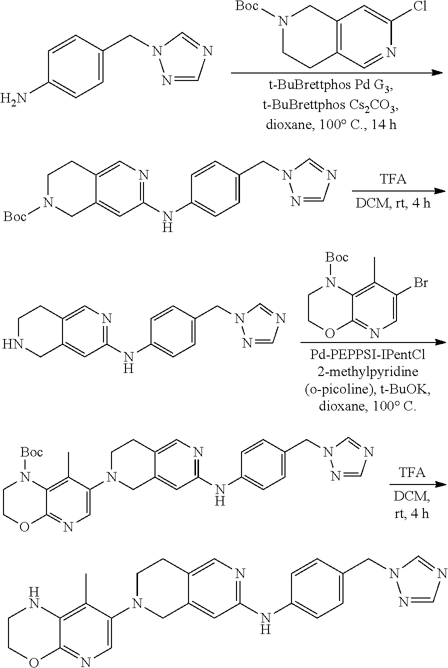

tert-butyl 7-({4-[(1H-1,2,4-triazol-1-yl)methyl]phenyl}amino)-1,2,3,4-tetrahydro-2,6-naphthyridine-2-carboxylate: To a stirred mixture of tert-butyl 7-chloro-1,2,3,4-tetrahydro-2,6-naphthyridine-2-carboxylate (1.50 g; 5.30 mmol) and 4-[(1H-1,2,4-triazol-1-yl)methyl]aniline (1458.50 mg; 7.95 mmol) in Dioxane-1,4 (20.00 ml) was added t-BuBrettPhos Pd G₃ (477.47 mg; 0.53 mmol), t-BuBrettPhos (270.54 mg; 0.53 mmol) and Cs₂CO₃ (5455.80 mg; 15.91 mmol) in portions at room temperature under nitrogen atmosphere. The resulting mixture was stirred for 2 h at 100° C. under nitrogen atmosphere. The resulting mixture was extracted with EA (3×50 mL). The combined organic layers were washed with brine, dried over anhydrous MgSO₄. After filtration, the filtrate was concentrated under reduced pressure. The residue was purified by C18 flash column eluted with 85% ACN in Water to afford tert-butyl 7-({4-[(1H-1,2,4-triazol-1-yl)methyl]phenyl}amino)-1,2,3,4-tetrahydro-2,6-naphthyridine-2-carboxylate (1.60 g; 74.2%) as a yellow solid.

N-{4-[(1H-1,2,4-triazol-1-yl)methyl]phenyl}-5,6,7,8-tetrahydro-2,6-naphthyridin-3-amine: To a stirred mixture of tert-butyl 7-({4-[(TH-1,2,4-triazol-1-yl)methyl]phenyl}amino)-1,2,3,4-tetrahydro-2,6-naphthyridine-2-carboxylate (1.10 g; 2.71 mmol) in DCM (16.00 ml) was added TFA (4.00 ml) in portions at room temperature under nitrogen atmosphere. The resulting mixture was stirred for 2 h at room temperature under nitrogen atmosphere. The pH value of the solution was adjusted to 9 with NaHCO₃. Then was extracted with EA (3×50 mL). The combined organic layers were washed with brine, dried over anhydrous MgSO₄. After filtration, the filtrate was concentrated under reduced pressure. The residue was purified by C18 flash column eluted with 40% ACN in Water to afford N-{4-[(1H-1,2,4-triazol-1-yl)methyl]phenyl}-5,6,7,8-tetrahydro-2,6-naphthyridin-3-amine (600.00 mg; 72.4%) as a yellow solid.

tert-butyl 8-methyl-7-[7-({4-[(1H-1,2,4-triazol-1-yl)methyl]phenyl}amino)-1,2,3,4-tetrahydro-2,6-naphthyridin-2-yl]-1H,2H,3H-pyrido[2,3-b][1,4]oxazine-1-carboxylate: To a stirred solution of N-{4-[(1H-1,2,4-triazol-1-yl)methyl]phenyl}-5,6,7,8-tetrahydro-2,6-naphthyridin-3-amine (400.00 mg; 1.31 mmol) and tert-butyl 7-bromo-8-methyl-1H,2H,3H-pyrido[2,3-b][1,4]oxazine-1-carboxylate (644.00 mg; 1.96 mmol) in 1,4-dioxane (5.00 ml) were added Pd-PEPPSI-IPentCl 2-methylpyridine (o-picoline) (110.00 mg; 0.12 mmol) and t-BuOK (440 mg; 3.92 mmol) in portions at room temperature under N₂ atmosphere. The resulting mixture was stirred for 2 h at 100° C. under N₂ atmosphere. The resulting mixture was diluted with water and extracted with EtOAc. The combined organic layers were washed with brine, dried over anhydrous Na₂SO₄. The resulting mixture was concentrated under reduced pressure. The residue was purified by silica gel column, eluted with 9% MeOH in DCM to afford tert-butyl 8-methyl-7-[7-({4-[(1H-1,2,4-triazol-1-yl)methyl]phenyl}amino)-1,2,3,4-tetrahydro-2,6-naphthyridin-2-yl]-1H,2H,3H-pyrido[2,3-b][1,4]oxazine-1-carboxylate (350.00 mg; 43.6%) as a yellow solid.

6-{8-methyl-1H,2H,3H-pyrido[2,3-b][1,4]oxazin-7-yl}-N-{4-[(1H-1,2,4-triazol-1-yl)methyl]phenyl}-5,6,7,8-tetrahydro-2,6-naphthyridin-3-amine: To a stirred solution of tert-butyl 8-methyl-7-[7-({4-[(1H-1,2,4-triazol-1-yl)methyl]phenyl}amino)-1,2,3,4-tetrahydro-2,6-naphthyridin-2-yl]-1H,2H,3H-pyrido[2,3-b][1,4]oxazine-1-carboxylate (300.00 mg; 0.49 mmol) in DCM (3.00 ml) was added TFA (1.00 ml) dropwise at 0° C. under N₂ atmosphere. The resulting mixture was stirred for 2 h at room temperature under N₂ atmosphere. The resulting mixture was diluted with DCM, the pH value of the solution was adjusted to 8 with NaHCO₃. Then extracted with DCM. The combined organic layers were washed with brine, dried over anhydrous Na₂SO₄. The resulting mixture was concentrated under reduced pressure. The residue was purified by Prep-HPLC with following condition: Column: Xselect CSH C18 OBD Column 30*150 mm 5 μm, n; Mobile Phase A: 10 mmol NH₄HCO₃+0.05% NH₄OH, Mobile Phase B: ACN; Flow rate: 60 mL/min; Gradient: 24% B to 50% B in 12 min, Wavelength: 254/220 nm; RT1 (min): 7.72; Number Of Runs: 4 to afford 6-{8-methyl-1H,2H,3H-pyrido[2,3-b][1,4]oxazin-7-yl}-N-{4-[(1H-1,2,4-triazol-1-yl)methyl]phenyl}-5,6,7,8-tetrahydro-2,6-naphthyridin-3-amine (133.10 mg; 59.8%) as a yellow solid.

HPLC: 99.77% purity, RT=2.87 min. MS: m/z=455.15 [M+H]⁺. ¹H NMR (300 MHz, DMSO-d6) δ 7.75-7.66 (m, 1H), 7.17 (ddd, J=14.0, 8.1, 4.0 Hz, 2H), 6.72-6.58 (m, 2H), 6.46 (dt, J=8.6, 3.7 Hz, 3H), 5.80 (d, J=5.2 Hz, 1H), 4.60-4.51 (m, 2H), 3.60-3.41 (m, 2H), 3.20 (d, J=4.7 Hz, 2H), 2.68-2.56 (m, 2H), 2.37 (d, J=9.7 Hz, 2H), 2.07 (s, 2H), 1.32 (q, J=4.8, 3.7 Hz, 3H).

### Example 145: Synthesis of Compound 7-{8-ethyl-1H,2H,3H-pyrido[2,3-b][1,4]oxazin-7-yl}-N-[4-(methanesulfonylmethyl)phenyl]-5H,6H,7H,8H-pyrido[3,4-d]pyrimidin-2-amine

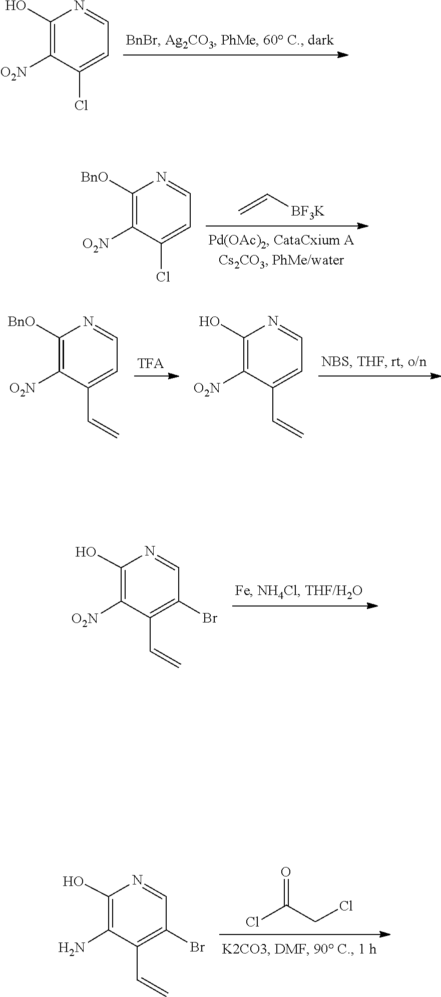

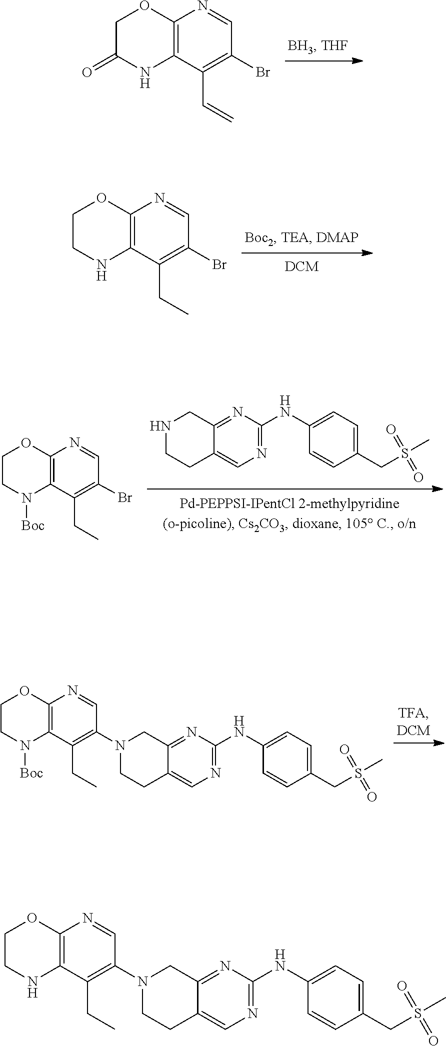 [SMILES: C=Cc1c(Br)cnc2c1NC(=O)CO2 ; CCc1c(Br)cnc2c1NCCO2 ; CCc1c(Br)cnc2c1N(C(=O)OC(C)(C)C)CCO2 ; CS(=O)(=O)Cc1ccc(Nc2ncc3c(n2)CNCC3)cc1 ; CCc1c(N2CCc3cnc(Nc4ccc(CS(C)(=O)=O)cc4)nc3C2)cnc2c1N(C(=O)OC(C)(C)C)CCO2 ; CCc1c(N2CCc3cnc(Nc4ccc(CS(C)(=O)=O)cc4)nc3C2)cnc2c1NCCO2]

2-(benzyloxy)-4-chloro-3-nitropyridine: To a stirred solution of 4-chloro-3-nitropyridin-2-ol (50.00 g; 272.14 mmol) and (bromomethyl)benzene (58.79 g; 326.57 mmol) in Toluene (500.00 ml) was added Ag₂CO₃ (94.79 g; 326.57 mmol) in portions at room temperature under nitrogen atmosphere. The resulting mixture was stirred for overnight at 60° C. The resulting mixture was allowed to cool down to room temperature. The resulting mixture was filtered, the filter cake was washed with MeOH (3×200 mL). The filtrate was concentrated under reduced pressure. The residue was purified by silica gel column chromatography, eluted with PE:EtOAc (9:1) to afford 2-(benzyloxy)-4-chloro-3-nitropyridine (50.00 g; 66.1%) as an off-white solid.

2-(benzyloxy)-4-ethenyl-3-nitropyridine: To a stirred solution of 2-(benzyloxy)-4-chloro-3-nitropyridine (50.00 g; 163.75 mmol) and potassium ethenyltrifluoroboranuide (44.31 g; 327.51 mmol) in Toluene (500.00 ml) and H₂O (150.00 ml) were added Pd(OAc)₂ (774.00 mg; 3.28 mmol), Butyldi-1-adamantylphosphine (1.78 g; 4.91 mmol) and Cs₂CO₃ (161.68 g; 491.26 mmol) at room temperature under nitrogen atmosphere. The resulting mixture was stirred for overnight at 110° C. The resulting mixture was allowed to cool down to room temperature. The resulting mixture was concentrated under reduced pressure. The residue was purified by silica gel column chromatography, eluted with PE:EtOAc (95:5) to afford 2-(benzyloxy)-4-ethenyl-3-nitropyridine (40.00 g; 88.9%) as a light yellow oil.

4-ethenyl-3-nitropyridin-2-ol: A solution of 2-(benzyloxy)-4-ethenyl-3-nitropyridine (40.00 g; 143.93 mmol) in TFA (40.00 ml) was stirred for overnight at room temperature under nitrogen atmosphere. The resulting mixture was concentrated under reduced pressure. The resulting mixture was extracted with EtOAc (3×500 mL). The combined organic layers were washed with brine, dried over anhydrous Na₂SO₄. After filtration, the filtrate was concentrated under reduced pressure to afford 4-ethenyl-3-nitropyridin-2-ol (20.00 g; 82.7%) as a yellow solid.

5-bromo-4-ethenyl-3-nitropyridin-2-ol: To a stirred solution of 4-ethenyl-3-nitropyridin-2-ol (20.00 g; 119.00 mmol) in THF (200.00 ml) was added NBS (21.84 g; 119.00 mmol) in portions at room temperature under nitrogen atmosphere. The resulting mixture was stirred for overnight at room temperature. The resulting mixture was extracted with EtOAc (3×300 bmL). The combined organic layers were washed with brine, dried over anhydrous Na₂SO₄. After filtration, the filtrate was concentrated under reduced pressure. The residue was purified by silica gel column chromatography, eluted with DCM:MeOH (98:2) to afford 5-bromo-4-ethenyl-3-nitropyridin-2-ol (23.00 g; 75.6%) as a yellow solid.

3-amino-5-bromo-4-ethenylpyridin-2-ol: To a stirred solution of 5-bromo-4-ethenyl-3-nitropyridin-2-ol (23.00 g; 89.92 mmol) in THF (200.00 ml) were added Fe (26.50 g; 449.62 mmol) and NH₄Cl (29.15 g; 539.54 mmol) (dissolved in H₂O (40.00 ml)) at room temperature under nitrogen atmosphere. The resulting mixture was stirred for overnight at room temperature. The resulting mixture was filtered, the filtered cake was washed with MeOH (3×200 mL). The filtrate was concentrated under reduced pressure to afford 3-amino-5-bromo-4-ethenylpyridin-2-ol (14.00 g; 61.5%) as a brown yellow solid.

7-bromo-8-ethenyl-1H,2H,3H-pyrido[2,3-b][1,4]oxazin-2-one: To a stirred solution of 3-amino-5-bromo-4-ethenylpyridin-2-ol (14.00 g; 62.24 mmol) in DMF (140.00 ml) were added 2-chloroacetyl chloride (11.10 g; 93.37 mmol) and K₂CO₃ (27.16 g; 186.73 mmol) at 0° C. under nitrogen atmosphere. The resulting mixture was stirred for 1 h at 90° C. The resulting mixture was allowed to cool down to room temperature. The resulting mixture was extracted with EtOAc (3×200 mL). The combined organic layers were washed with brine, dried over anhydrous Na₂SO₄. After filtration, the filtrate was concentrated under reduced pressure. The residue was purified by silica gel column chromatography, eluted with PE:EtOAc (2:1) to afford 7-bromo-8-ethenyl-1H,2H,3H-pyrido[2,3-b][1,4]oxazin-2-one (9.00 g; 55.7%) as a light yellow solid.

7-bromo-8-ethyl-1H,2H,3H-pyrido[2,3-b][1,4]oxazine: To a stirred solution of 7-bromo-8-ethenyl-1H,2H,3H-pyrido[2,3-b][1,4]oxazin-2-one (1.00 g; 3.85 mmol) in THF (3.00 ml) was added BH₃ in THF (19.25 ml; 19.25 mmol) at 0° C. under nitrogen atmosphere. The resulting mixture was stirred for 2 h at room temperature. The reaction was quenched with water at 0° C. The resulting mixture was concentrated under reduced pressure. The residue was purified by reverse flash chromatography with the following conditions: column, C18 silica gel; mobile phase, ACN in water, 50% to 60% gradient in 20 min; detector, UV 254 nm to afford 7-bromo-8-ethyl-1H,2H,3H-pyrido[2,3-b][1,4]oxazine (500.00 mg; 51.8%) as a yellow oil.

tert-butyl 7-bromo-8-ethyl-1H,2H,3H-pyrido[2,3-b][1,4]oxazine-1-carboxylate: To a stirred solution of 7-bromo-8-ethyl-1H,2H,3H-pyrido[2,3-b][1,4]oxazine (480.00 mg; 1.91 mmol) and Boc₂O (1.27 g; 5.74 mmol) in DCM (5.00 ml) were added TEA (0.84 ml; 5.75 mmol) and DMAP (45.00 mg; 0.38 mmol) at 0° C. under nitrogen atmosphere. The resulting mixture was stirred for overnight at room temperature. The resulting mixture was concentrated under reduced pressure. The residue was purified by silica gel column chromatography, eluted with DCM:MeOH (98:2) to afford tert-butyl 7-bromo-8-ethyl-1H,2H,3H-pyrido[2,3-b][1,4]oxazine-1-carboxylate (450.00 mg; 65.7%) as a yellow brown oil.

tert-butyl 8-ethyl-7-(2-{[4-(methanesulfonylmethyl)phenyl]amino}-5H,6H,7H,8H-pyrido[3,4-d]pyrimidin-7-yl)-1H,2H,3H-pyrido[2,3-b][1,4]oxazine-1-carboxylate: To a stirred solution of tert-butyl 7-bromo-8-ethyl-1H,2H,3H-pyrido[2,3-b][1,4]oxazine-1-carboxylate (300.00 mg; 0.84 mmol) and N-[4-(methanesulfonylmethyl)phenyl]-5H,6H,7H,8H-pyrido[3,4-d]pyrimidin-2-amine (230.00 mg; 0.69 mmol) in Dioxane-1,4 (5.00 ml) were added Pd-PEPPSI-IPentCl 2-methylpyridine (o-picoline) (30.00 mg; 0.03 mmol) and Cs₂CO₃ (678.00 mg; 2.06 mmol) in portions at room temperature under nitrogen atmosphere. The resulting mixture was stirred for overnight at 100° C. The resulting mixture was allowed to cool down to room temperature. The resulting mixture was extracted with EtOAc (3×20 mL). The combined organic layers were washed with brine, dried over anhydrous Na₂SO₄. After filtration, the filtrate was concentrated under reduced pressure. The residue was purified by reverse flash chromatography with the following conditions: column, C18 silica gel; mobile phase, ACN in water (0.05% TFA), 40% to 50% gradient in 20 min; detector, UV 254 nm to afford tert-butyl 8-ethyl-7-(2-{[4-(methanesulfonylmethyl)phenyl]amino}-5H,6H,7H,8H-pyrido[3,4-d]pyrimidin-7-yl)-1H,2H,3H-pyrido[2,3-b][1,4]oxazine-1-carboxylate (120.00 mg; 26.3%) as a yellow solid.

7-{8-ethyl-1H,2H,3H-pyrido[2,3-b][1,4]oxazin-7-yl}-N-[4-(methanesulfonylmethyl)phenyl]-5H,6H,7H,8H-pyrido[3,4-d]pyrimidin-2-amine: To a stirred solution of tert-butyl 8-ethyl-7-(2-{[4-(methanesulfonylmethyl) phenyl]amino}-5H,6H,7H,8H-pyrido[3,4-d]pyrimidin-7-yl)-1H,2H,3H-pyrido[2,3-b][1,4]oxazine-1-carboxylate (100.00 mg; 0.15 mmol) in DCM (3.00 ml) was added TFA (1.00 ml) dropwise at 0° C. under nitrogen atmosphere. The resulting mixture was stirred for 2 h at room temperature. The resulting mixture was concentrated under reduced pressure. The crude product was purified by Prep-HPLC with the following conditions (Column: XBridge BEH C18 OBD Prep Column, 19*250 mm, 5 μm; Mobile Phase A: Water (10 mmol/L NH₄HCO₃+0.05% NH₄OH), Mobile Phase B: MeOH; Preparative; Flow rate: 20 mL/min; Gradient: 57% to 70% in 10 min; Wavelength: 254/220 nm; RT1 (min): 8.75/9.5) to afford 7-{8-ethyl-1H,2H,3H-pyrido[2,3-b][1,4]oxazin-7-yl}-N-[4-(methanesulfonylmethyl)phenyl]-5H,6H,7H,8H-pyrido[3,4-d]pyrimidin-2-amine (13.40 mg; 18.4%) as an off-white solid.

HPLC: 99.27% purity, RT=2.90 min. MS: m/z=481.25 [M+H]⁺. ¹H NMR (300 MHz, DMSO-d6) δ 9.63 (s, 1H), 8.35 (s, 1H), 7.77 (d, J=8.0 Hz, 2H), 7.39 (s, 1H), 7.28 (d, J=8.2 Hz, 2H), 5.58 (s, 1H), 4.37 (s, 2H), 4.20 (t, J=4.3 Hz, 2H), 3.96 (s, 2H), 3.29 (s, 2H), 3.12 (t, J=5.6 Hz, 2H), 2.86 (s, 3H), 2.80 (t, J=5.7 Hz, 2H), 2.59 (q, J=7.3 Hz, 2H), 1.06 (t, J=7.3 Hz, 3H).

### Example 146: Synthesis of Compound N-[4-(methanesulfonylmethyl)phenyl]-7-{1-methyl-1H-pyrrolo[2,3-b]pyridin-4-yl}-5H,6H,7H,8H-pyrido[3,4-d]pyrimidin-2-amine

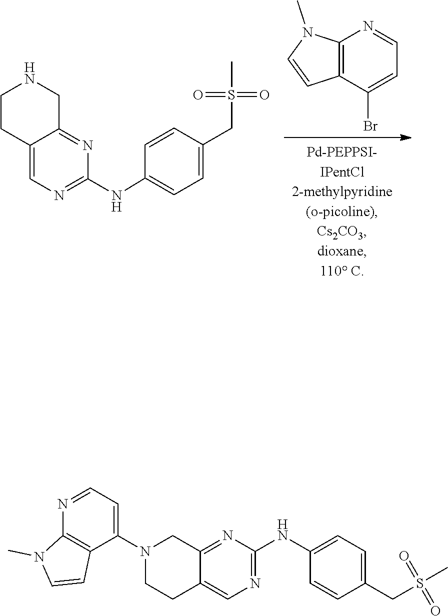

N-[4-(methanesulfonylmethyl)phenyl]-7-{1-methyl-1H-pyrrolo[2,3-b]pyridin-4-yl}-5H,6H,7H,8H-pyrido[3,4-d]pyrimidin-2-amine: To a stirred solution of 4-bromo-1-methyl-1H-pyrrolo[2,3-b]pyridine (327.00 mg; 1.49 mmol) and N-[4-(methanesulfonylmethyl)phenyl]-5H,6H,7H,8H-pyrido[3,4-d]pyrimidin-2-amine (500.00 mg; 1.49 mmol) in Dioxane-1,4 (1.00 ml) was added Pd-PEPPSI-IPentCl 2-methylpyridine (o-picoline) (1321.00 mg; 1.49 mmol) and Cs₂CO₃ (1024.00 mg; 2.99 mmol) at room temperature. The resulting mixture was stirred for 3 h at 100° C. under nitrogen atmosphere. The resulting mixture was concentrated under reduced pressure. The residue was purified by Prep-HPLC with following conditions (Column: X Select CSH Prep C18 OBD Column, 19*250 mm, 5 um; Mobile Phase A: Water (0.05% HCl), Mobile Phase B:ACN; Flow rate:20 mL/min; Gradient:18 B to 35 B in 9 min) to afford N-[4-(methanesulfonylmethyl)phenyl]-7-{1-methyl-1H-pyrrolo[2,3-b]pyridin-4-yl}-5H,6H,7H,8H-pyrido[3,4-d]pyrimidin-2-amine (18.80 mg; 2.72%) as a white solid.

HPLC: 96.9% purity, RT=2.710 min. MS: m/z=449.1 [M+H]⁺. ¹H NMR (400 MHz, DMSO-d6) δ 9.68 (s, 1H), 8.36 (s, 1H), 8.02 (d, J=5.4 Hz, 1H), 7.81 (d, J=8.1 Hz, 2H), 7.32 (t, J=5.5 Hz, 3H), 6.56 (dd, J=10.4, 4.6 Hz, 2H), 4.47 (d, J=63.4 Hz, 4H), 3.84 (t, J=5.7 Hz, 2H), 3.76 (s, 3H), 2.88 (s, 5H).

### Example 147: Synthesis of Compound N-{4-[2-(2-methoxyethoxy) ethanesulfonyl]phenyl}-7-{8-methyl-1H,2H,3H-pyrido[2,3-b][1,4]oxazin-7-yl}-5H,6H,7H,8H-pyrido[3,4-d]pyrimidin-2-amine

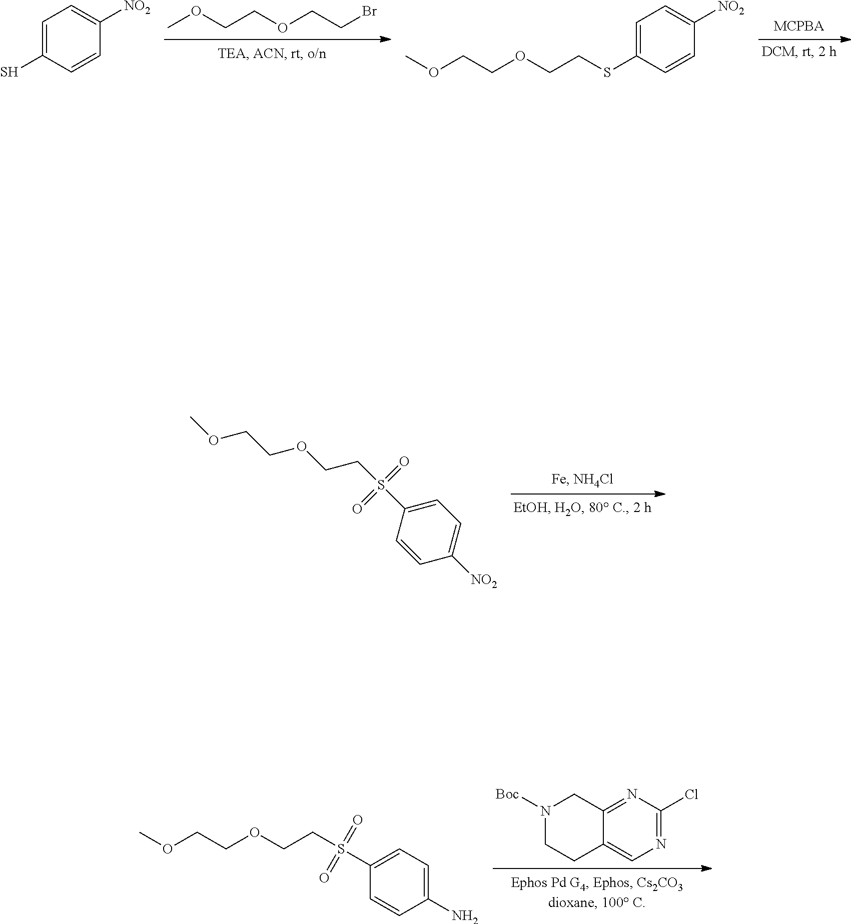

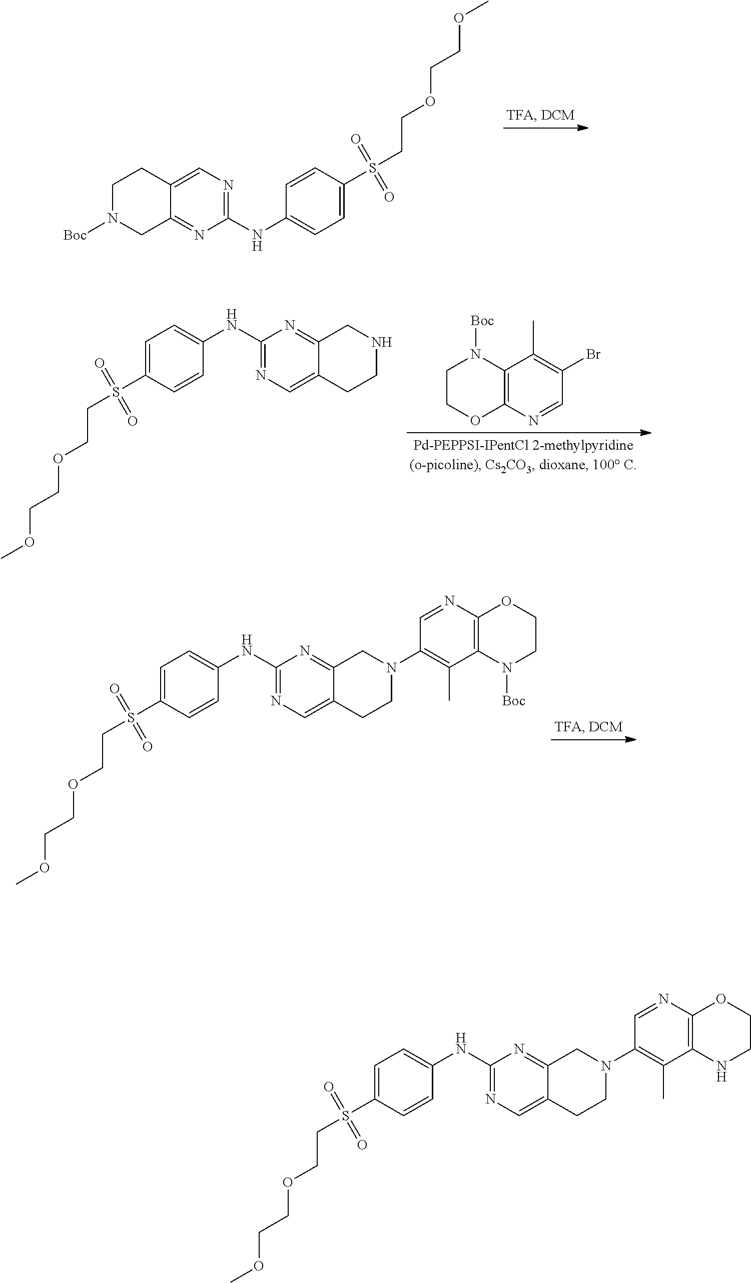 [SMILES: COCCOCCS(=O)(=O)c1ccc(Nc2ncc3c(n2)CN(C(=O)OC(C)(C)C)CC3)cc1 ; COCCOCCS(=O)(=O)c1ccc(Nc2ncc3c(n2)CNCC3)cc1 ; Cc1c(Br)cnc2c1N(C(=O)OC(C)(C)C)CCO2 ; COCCOCCS(=O)(=O)c1ccc(Nc2ncc3c(n2)CN(c2cnc4c(c2C)N(C(=O)OC(C)(C)C)CCO4)CC3)cc1 ; COCCOCCS(=O)(=O)c1ccc(Nc2ncc3c(n2)CN(c2cnc4c(c2C)NCCO4)CC3)cc1]

1-{[2-(2-methoxyethoxy)ethyl] sulfanyl}-4-nitrobenzene: To a stirred mixture of 4-nitrobenzene-1-thiol (2.00 g; 12.63 mmol) and 1-bromo-2-(2-methoxyethoxy) ethane (3.16 g; 16.42 mmol) in CH₃CN (20.00 ml) was added TEA (2.02 g; 18.95 mmol) in portions at 25° C. under nitrogen atmosphere. The resulting mixture was stirred for 2 h at 25° C. under nitrogen atmosphere. The resulting mixture was extracted with EA (3×50 mL). The combined organic layers were washed with brine, dried over anhydrous MgSO₄. After filtration, the filtrate was concentrated under reduced pressure. The residue was purified by silica gel column chromatography, eluted with EA:PE=7:93 to afford 1-{[2-(2-methoxyethoxy)ethyl]sulfanyl}-4-nitrobenzene (2.70 g; 78.4%) as an orange oil.

1-[2-(2-methoxyethoxy) ethanesulfonyl]-4-nitrobenzene: To a stirred mixture of 1-{[2-(2-methoxyethoxy)ethyl] sulfanyl}-4-nitrobenzene (1.40 g; 5.14 mmol) and m-CPBA (2.80 g; 15.41 mmol) in DCM (14.00 ml) at 25° C. under nitrogen atmosphere. The resulting mixture was stirred for 2 h at 25° C. under nitrogen atmosphere. The mixture was basified to pH=10 with Na₂CO₃. The resulting mixture was extracted with EA (3×50 mL). The combined organic layers were washed with brine, dried over anhydrous MgSO₄. After filtration, the filtrate was concentrated under reduced pressure to afford 1-[2-(2-methoxyethoxy) ethanesulfonyl]-4-nitrobenzene (1.80 g; 84.13%) as a white solid.

4-[2-(2-methoxyethoxy) ethanesulfonyl] aniline: To a stirred mixture of 1-[2-(2-methoxyethoxy) ethanesulfonyl]-4-nitrobenzene (900.00 mg; 2.62 mmol) and NH₄Cl (736.81 mg; 13.09 mmol) in ethanol (6.00 ml) and Water (4.00 ml) was added Fe (590.59 mg; 10.47 mmol) in portions at 25° C. under nitrogen atmosphere. The resulting mixture was stirred for 2 h at 80° C. under nitrogen atmosphere. The resulting mixture was extracted with EA (3×50 mL). The combined organic layers were washed with brine, dried over anhydrous MgSO₄. After filtration, the filtrate was concentrated under reduced pressure. The residue was purified by silica gel column chromatography, eluted with PE: EA=50:50 to afford 4-[2-(2-methoxyethoxy) ethanesulfonyl] aniline (577.00 mg; 85.1%) as a colorless oil.

tert-butyl 2-({4-[2-(2-methoxyethoxy) ethanesulfonyl]phenyl}amino)-5H,6H,7H,8H-pyrido[3,4-d]pyrimidine-7-carboxylate: To a stirred mixture of 4-[2-(2-methoxyethoxy) ethanesulfonyl] aniline (577.00 mg; 2.23 mmol) and tert-butyl 2-chloro-5H,6H,7H,8H-pyrido[3,4-d]pyrimidine-7-carboxylate (916.82 mg; 3.34 mmol) in Dioxane-1,4 (8.00 ml) was added Ephos (62.63 mg; 0.11 mmol), EPhos Pd G₄ (107.57 mg; 0.11 mmol) and Cs₂CO₃ (2289.34 mg; 6.68 mmol) in portions at 25° C. under nitrogen atmosphere. The resulting mixture was stirred for 2 h at 80° C. under nitrogen atmosphere. The residue was purified by silica gel column chromatography, eluted with PE:EA=25:75 to afford tert-butyl 2-({4-[2-(2-methoxyethoxy) ethanesulfonyl]phenyl}amino)-5H,6H,7H,8H-pyrido[3,4-d]pyrimidine-7-carboxylate (260.00 mg; 20.7%) as a yellow oil.

N-{4-[2-(2-methoxyethoxy) ethanesulfonyl]phenyl}-5H,6H,7H,8H-pyrido[3,4-d]pyrimidin-2-amine: To a stirred mixture of tert-butyl 2-({4-[2-(2-methoxyethoxy) ethanesulfonyl]phenyl}amino)-5H,6H,7H,8H-pyrido[3,4-d]pyrimidine-7-carboxylate (240.00 mg; 0.42 mmol) in DCM (2.00 ml) was added trifluoroacetic acid (0.50 ml) in portions at 25° C. under nitrogen atmosphere. The resulting mixture was stirred for 2 h at 25° C. under nitrogen atmosphere. The residue was purified by silica gel column chromatography, eluted with PE:EA=30:70 to afford N-{4-[2-(2-methoxyethoxy)ethanesulfonyl]phenyl}-5H,6H,7H,8H-pyrido[3,4-d]pyrimidin-2-amine (110.00 mg; 66.1%) as a yellow solid.

tert-butyl 7-[2-({4-[2-(2-methoxyethoxy)ethanesulfonyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate: To a stirred mixture of N-{4-[2-(2-methoxyethoxy)ethanesulfonyl]phenyl}-5H,6H,7H,8H-pyrido[3,4-d]pyrimidin-2-amine (900.00 mg; 2.29 mmol) and tert-butyl 7-bromo-8-methyl-1H,2H,3H-pyrido[2,3-b][1,4]oxazine-1-carboxylate (932.05 mg; 2.75 mmol) in Dioxane-1,4 (20.00 ml) was added Pd-PEPPSI-IPentCl 2-methylpyridine (o-picoline) (202.80 mg; 0.23 mmol) and Cs₂CO₃ (2359.42 mg; 6.88 mmol) in portions at 25° C. under nitrogen atmosphere. The resulting mixture was stirred for 2 h at 100° C. under nitrogen atmosphere. The resulting mixture was concentrated under reduced pressure. The residue was purified by silica gel column chromatography, eluted with EA:PE=60:40 to afford tert-butyl 7-[2-({4-[2-(2-methoxyethoxy) ethanesulfonyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate (200.00 mg; 13.6%) as a yellow oil.

N-{4-[2-(2-methoxyethoxy) ethanesulfonyl]phenyl}-7-{8-methyl-1H,2H,3H-pyrido[2,3-b][1,4]oxazin-7-yl}-5H,6H,7H,8H-pyrido[3,4-d]pyrimidin-2-amine: To a stirred mixture of tert-butyl 7-[2-({4-[2-(2-methoxyethoxy) ethanesulfonyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate (200.00 mg; 0.31 mmol) in DCM (8.00 ml) was added TFA (2.00 ml) in portions at 25° C. under nitrogen atmosphere. The resulting mixture was stirred for 1 h at 25° C. under nitrogen atmosphere. The resulting mixture was concentrated under vacuum. The crude product was purified by Prep-HPLC with the following conditions (Column: X Bridge Prep OBD C18 Column, 30*150 mm, 5?m; Mobile Phase A: Water (10 mmol/L NH₄HCO₃+0.05% NH₄OH), Mobile Phase B: ACN; Flow rate: 60 mL/min; Gradient: 20% B to 50% B in 9 min, Wavelength: 254 nm; RT1 (min): 7; Number Of Runs: 2) to afford N-{4-[2-(2-methoxyethoxy)ethanesulfonyl]phenyl}-7-{8-methyl-1H,2H,3H-pyrido[2,3-b][1,4]oxazin-7-yl}-5H,6H,7H,8H-pyrido[3,4-d]pyrimidin-2-amine (31.80 mg; 18.2%) as a light yellow solid.

HPLC: 96.8% purity, RT=3.01 min. MS: m/z=541.3 [M+H]⁺. ¹H NMR (400 MHz, DMSO-d6) δ 10.13 (s, 1H), 8.44 (s, 1H), 8.03-7.95 (m, 2H), 7.79-7.71 (m, 2H), 7.29 (s, 1H), 5.52 (t, J=2.7 Hz, 1H), 4.22-4.16 (m, 2H), 4.01 (s, 2H), 3.66 (t, J=6.1 Hz, 2H), 3.49 (t, J=6.1 Hz, 2H), 3.42-3.35 (m, 2H), 3.32-3.24 (m, 4H), 3.16 (s, 5H), 2.83 (t, J=5.8 Hz, 2H), 2.04 (s, 3H).

### Example 148: Synthesis of Compound 2-{4-[(7-{8-methyl-1H,2H,3H-pyrido[2,3-b]oxazin-7-yl}-5H,6H,7H,8H-pyrido[3,4-d]pyrimidin-2-yl) amino]phenyl}acetonitrile; formic acid

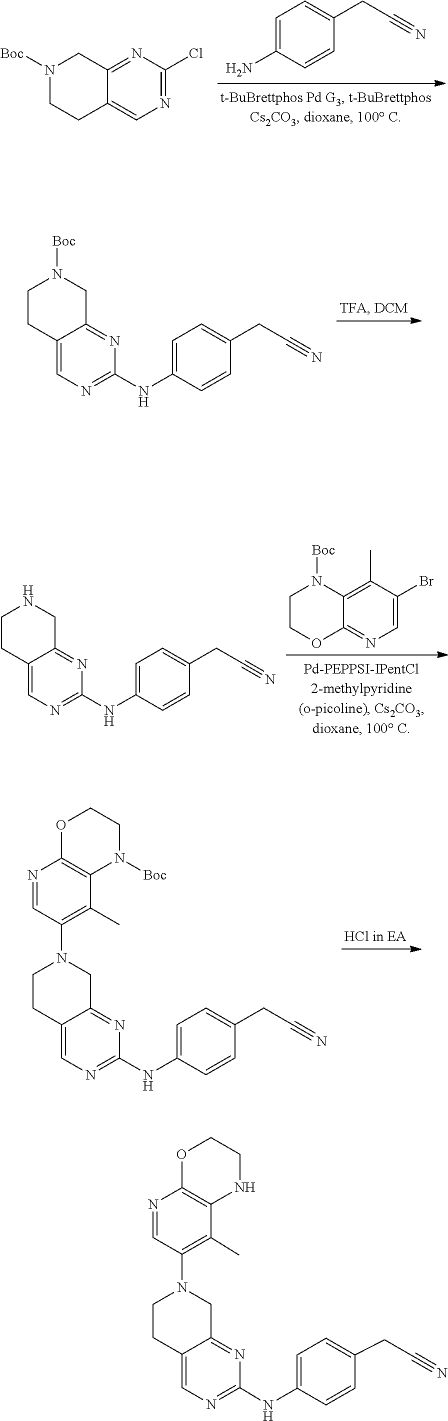

tert-butyl 2-{[4-(cyanomethyl)phenyl]amino}-5H,6H,7H,8H-pyrido[3,4-d]pyrimidine-7-carboxylate: To a stirred mixture of tert-butyl 2-chloro-5H,6H,7H,8H-pyrido[3,4-d]pyrimidine-7-carboxylate (850.00 mg; 3.09 mmol) and 2-(4-aminophenyl) acetonitrile (645.71 mg; 4.64 mmol) in Dioxane-1,4 (12.00 ml) was added t-BuBrettPhos Pd G₃ (278.63 mg; 0.31 mmol), t-BuBrettPhos (315.74 mg; 0.62 mmol) and Cs₂CO₃ (3055.10 mg; 9.28 mmol) in portions at 25° C. under nitrogen atmosphere. The resulting mixture was stirred for 2 h at 100° C. under nitrogen atmosphere. The resulting mixture was extracted with EA (3×50 mL). The combined organic layers were washed with brine, dried over anhydrous MgSO₄. After filtration, the filtrate was concentrated under reduced pressure. The residue was purified by silica gel column chromatography, eluted with EA:PE=25:75 to afford tert-butyl 2-{[4-(cyanomethyl)phenyl]amino}-5H,6H,7H,8H-pyrido[3,4-d]pyrimidine-7-carboxylate (1.00 g; 88.4%) as a yellow solid.

2-[4-({5H,6H,7H,8H-pyrido[3,4-d]pyrimidin-2-yl}amino)phenyl]acetonitrile: To a stirred mixture of tert-butyl 2-{[4-(cyanomethyl)phenyl]amino}-5H,6H,7H,8H-pyrido[3,4-d]pyrimidine-7-carboxylate (1.00 g; 2.74 mmol) in dichloromethane (20.00 ml) was added TFA (5.00 ml) in portions at 25 degrees C. under nitrogen atmosphere. The resulting mixture was stirred for 2 h at 25 degrees C. under nitrogen atmosphere. The resulting mixture was concentrated under reduced pressure. The residue was purified by silica gel column chromatography, eluted with EA:PE=50:50 to afford 2-[4-({5H,6H,7H,8H-pyrido[3,4-d]pyrimidin-2-yl}amino) phenyl] acetonitrile (600.00 mg; 82.1%) as a yellow solid.

tert-butyl 7-(2-{[4-(cyanomethyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate: To a stirred mixture of tert-butyl 7-bromo-8-methyl-1H,2H,3H-pyrido[2,3-b][1,4]oxazine-1-carboxylate (350.00 mg; 1.06 mmol) and 2-[4-({5H,6H,7H,8H-pyrido[3,4-d]pyrimidin-2-yl}amino)phenyl]acetonitrile (369.07 mg; 1.38 mmol) in Dioxane-1,4 (16.00 ml) was added Pd-PEPPSI-IPentCl 2-methylpyridine (o-picoline) (94.03 mg; 0.11 mmol) and Cs₂CO₃ (1.09 g; 3.19 mmol;) in portions at 25° C. under nitrogen atmosphere. The resulting mixture was stirred for 2 h at 100° C. under nitrogen atmosphere. The resulting mixture was concentrated under reduced pressure. The residue was purified by silica gel column chromatography, eluted with EA:PE=52:48 to afford tert-butyl 7-(2-{[4-(cyanomethyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (430.00 mg; 75.6%) as a yellow solid.

2-{4-[(7-{8-methyl-1H,2H,3H-pyrido[2,3-b][1,4]oxazin-7-yl}-5H,6H,7H,8H-pyrido[3,4-d]pyrimidin-2-yl) amino]phenyl}acetonitrile; formic acid: A mixture of tert-butyl 7-(2-{[4-(cyanomethyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (430.00 mg; 0.80 mmol) in hydrochloric acid 4.0 M Solution in ethyl acetate (20.00 ml) was stirred for 2 h at room temperature under nitrogen atmosphere. The resulting mixture was concentrated under vacuum. The crude product was purified by Prep-HPLC with the following conditions (Column: X select CSH C18 OBD Column 30*150 mm 5 um, n; Mobile Phase A: Water (0.1% FA), Mobile Phase B: ACN; Flow rate: 60 mL/min; Gradient: 15% B to 45% B in 8 min, Wavelength: 254 nm; RT1 (min): 7;) to afford 2-{4-[(7-{8-methyl-1H,2H,3H-pyrido[2,3-b][1,4]oxazin-7-yl}-5H,6H,7H,8H-pyrido[3,4-d]pyrimidin-2-yl) amino]phenyl}acetonitrile; formic acid (64.10 mg; 17.0%) as a yellow solid.

HPLC: 97.8% purity, RT=3.20 min. MS: m/z=414.2 [M+H]⁺. ¹H NMR (300 MHz, DMSO-d6) δ 9.60 (s, 1H), 8.14 (s, 1H), 7.81-7.72 (m, 2H), 7.29 (s, 1H), 7.27-7.18 (m, 2H), 5.53 (s, 1H), 4.19 (t, J=4.3 Hz, 2H), 3.95 (d, J=10.7 Hz, 4H), 3.30 (s, 2H), 3.12 (t, J=5.6 Hz, 2H), 2.80 (d, J=5.5 Hz, 2H), 2.04 (s, 3H).

### Example 149: Synthesis of Compound 2-{2-methyl-4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}acetonitrile

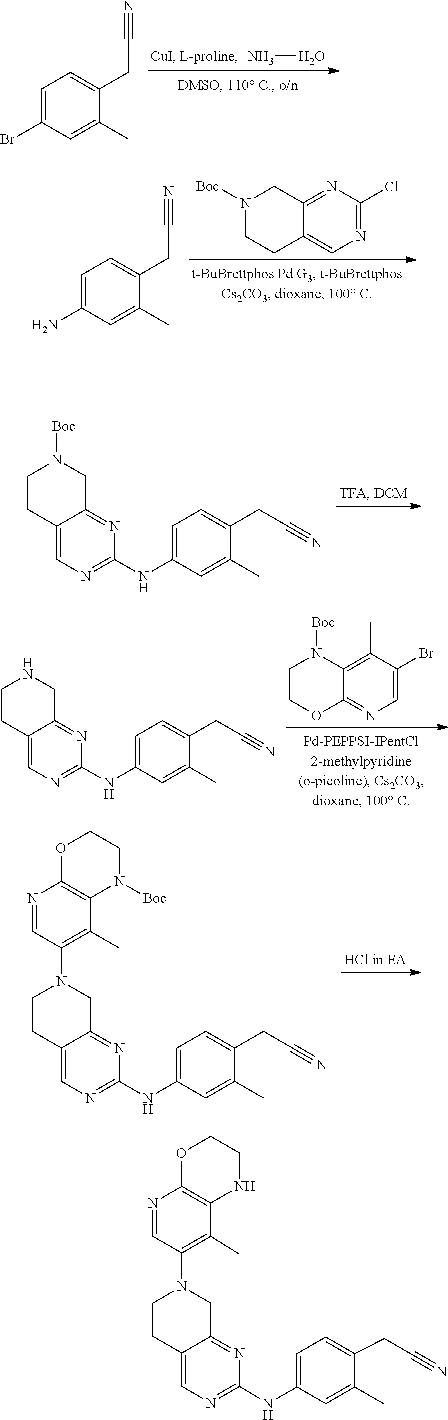

2-(4-amino-2-methylphenyl) acetonitrile: To a stirred solution of 2-(4-amino-2-methylphenyl) acetonitrile (2.00 g; 12.31 mmol) in (DMSO (15.00 ml) and NH₄OH (3.00 ml) were added CuI (4.94 g; 24.63 mmol), L-Proline (0.21 g; 1.85 mmol) and K₂CO₃ (0.54 g; 3.69 mmol) in portions at room temperature under N₂ atmosphere. The resulting mixture was stirred for overnight at 110° C. under N₂ atmosphere. The resulting mixture was diluted with water (50 mL), extracted with EtOAc (3×100 mL). The combined organic layers were washed with brine (150 mL), dried over anhydrous Na₂SO₄. The resulting mixture was concentrated under reduced pressure. The residue was purified by silica gel column, eluted with 60% EtOAc in PE to afford crude product. The crude product was repurified by C18 flash column eluted with 45% ACN in Water to afford 2-(4-amino-2-methylphenyl) acetonitrile (740.00 mg; 32.6%) as a yellow oil.

tert-butyl 2-{[4-(cyanomethyl)-3-methylphenyl]amino}-5H,6H,7H,8H-pyrido[3,4-d]pyrimidine-7-carboxylate: To a stirred mixture of tert-butyl 2-chloro-5H,6H,7H,8H-pyrido[3,4-d]pyrimidine-7-carboxylate (500.00 mg; 1.81 mmol) and 2-(4-amino-2-methylphenyl) acetonitrile (500.06 mg; 2.71 mmol) in Dioxane-1,4 (12.00 ml) was added t-BuBrettPhos Pd G₃ (162.71 mg; 0.18 mmol), t-BuBrettPhos (184.39 mg; 0.36 mmol) and Cs₂CO₃ (1.78 g; 5.42 mmol) in portions at 25° C. under nitrogen atmosphere. The resulting mixture was stirred for 2 h at 100° C. under nitrogen atmosphere. The resulting mixture was extracted with EA (3×50 mL). The combined organic layers were washed with brine, dried over anhydrous MgSO₄. After filtration, the filtrate was concentrated under reduced pressure. The residue was purified by silica gel column chromatography, eluted with EA:PE=25:75 to afford tert-butyl 2-{[4-(cyanomethyl)-3-methylphenyl]amino}-5H,6H,7H,8H-pyrido[3,4-d]pyrimidine-7-carboxylate (320.00 mg; Crude) as a yellow solid.

2-[2-methyl-4-({5H,6H,7H,8H-pyrido[3,4-d]pyrimidin-2-yl}amino)phenyl] acetonitrile: To a stirred mixture of tert-butyl 2-{[4-(cyanomethyl)-3-methylphenyl]amino}-5H,6H,7H,8H-pyrido[3,4-d]pyrimidine-7-carboxylate (300.00 mg; 0.79 mmol) in DCM (10.00 ml) was added TFA (2.50 ml) in portions at 25° C. under nitrogen atmosphere. The resulting mixture was stirred for 2 h at 25° C. under nitrogen atmosphere. The resulting mixture was extracted with EA (3×50 mL). The combined organic layers were washed with brine, dried over anhydrous MgSO₄. After filtration, the filtrate was concentrated under reduced pressure. The residue was purified by silica gel column chromatography, eluted with EA:PE=50:50 to afford 2-[2-methyl-4-({5H,6H,7H,8H-pyrido[3,4-d]pyrimidin-2-yl}amino)phenyl]acetonitrile (320.00 mg; 96.8%) as a yellow solid.

tert-butyl 7-(2-{[4-(cyanomethyl)-3-methylphenyl]amino}-5H,6H,7H,8H-pyrido[3,4-d]pyrimidin-7-yl)-8-methyl-1H,2H,3H-pyrido[2,3-b]oxazine-1-carboxylate: To a stirred mixture of tert-butyl 7-bromo-8-methyl-1H,2H,3H-pyrido[2,3-b][1,4]oxazine-1-carboxylate (314.05 mg; 0.95 mmol) and 2-[2-methyl-4-({5H,6H,7H,8H-pyrido[3,4-d]pyrimidin-2-yl}amino)phenyl]acetonitrile (220.00 mg; 0.73 mmol) in 1,4-dioxane (1.00 ml) was added Pd-PEPPSI-IPentCl 2-methylpyridine (o-picoline) (64.34 mg; 0.07 mmol) and Cs₂CO₃ (718.26 mg; 2.18 mmol) in portions at 25° C. under nitrogen atmosphere. The resulting mixture was stirred for 2 h at 100° C. under nitrogen atmosphere. The resulting mixture was extracted with EA (3×50 mL). The combined organic layers were washed with brine, dried over anhydrous MgSO₄. After filtration, the filtrate was concentrated under reduced pressure. The residue was purified by silica gel column chromatography, eluted with EA:PE=50:50 to afford tert-butyl 7-(2-{[4-(cyanomethyl)-3-methylphenyl]amino}-5H,6H,7H,8H-pyrido[3,4-d]pyrimidin-7-yl)-8-methyl-1H,2H,3H-pyrido[2,3-b][1,4]oxazine-1-carboxylate (320.00 mg; 83.4%) as an orange solid.

2-{2-methyl-4-[(7-{8-methyl-1H,2H,3H-pyrido[2,3-b][1,4]oxazin-7-yl}-5H,6H,7H,8H-pyrido[3,4-d]pyrimidin-2-yl) amino]phenyl}acetonitrile; formic acid: A mixture of tert-butyl 7-(2-{[4-(cyanomethyl)-3-methylphenyl]amino}-5H,6H,7H,8H-pyrido[3,4-d]pyrimidin-7-yl)-8-methyl-1H,2H,3H-pyrido[2,3-b][1,4]oxazine-1-carboxylate (300.00 mg; 0.57 mmol) in hydrochloric acid 4.0 M solution in ethyl acetate (20.00 ml) at 25° C. under nitrogen atmosphere. The resulting mixture was stirred for 2 h at 25° C. under nitrogen atmosphere. The resulting mixture was concentrated under vacuum. The crude product was purified by Prep-HPLC with the following conditions (Column: X select CSH C18 OBD Column 30*150 mm 5 um, n; Mobile Phase A: Water (0.1% FA), Mobile Phase B: ACN; Flow rate: 60 mL/min; Gradient: 15% B to 45% B in 8 min, Wavelength: 254 nm; RT1 (min): 7) to afford 2-{2-methyl-4-[(7-{8-methyl-1H,2H,3H-pyrido[2,3-b][1,4]oxazin-7-yl}-5H,6H,7H,8H-pyrido[3,4-d]pyrimidin-2-yl) amino]phenyl}acetonitrile (22.00 mg; 7.2%) as a yellow solid.

HPLC: 88.4% purity, RT=4.77 min. MS: m/z=428.2 [M+H]⁺. ¹H NMR (300 MHz, DMSO-d6) δ 9.51 (s, 1H), 8.34 (s, 1H), 7.64 (dd, J=8.3, 2.3 Hz, 1H), 7.59 (d, J=2.3 Hz, 1H), 7.29 (s, 1H), 7.20 (d, J=8.4 Hz, 1H), 5.52 (d, J=2.6 Hz, 1H), 4.19 (dd, J=5.2, 3.4 Hz, 2H), 3.96 (s, 2H), 3.88 (s, 2H), 3.36-3.28 (m, 2H), 3.12 (t, J=5.7 Hz, 2H), 2.79 (t, J=5.6 Hz, 2H), 2.26 (s, 3H), 2.04 (s, 3H).

### Example 150: Synthesis of Compound N,N-dimethyl-1-{4-[(6-{8-methyl-1H,2H,3H-pyrido[2,3-b][1,4]oxazin-7-yl}-5,6,7,8-tetrahydro-2,6-naphthyridin-3-yl)amino]phenyl}methanesulfonamide

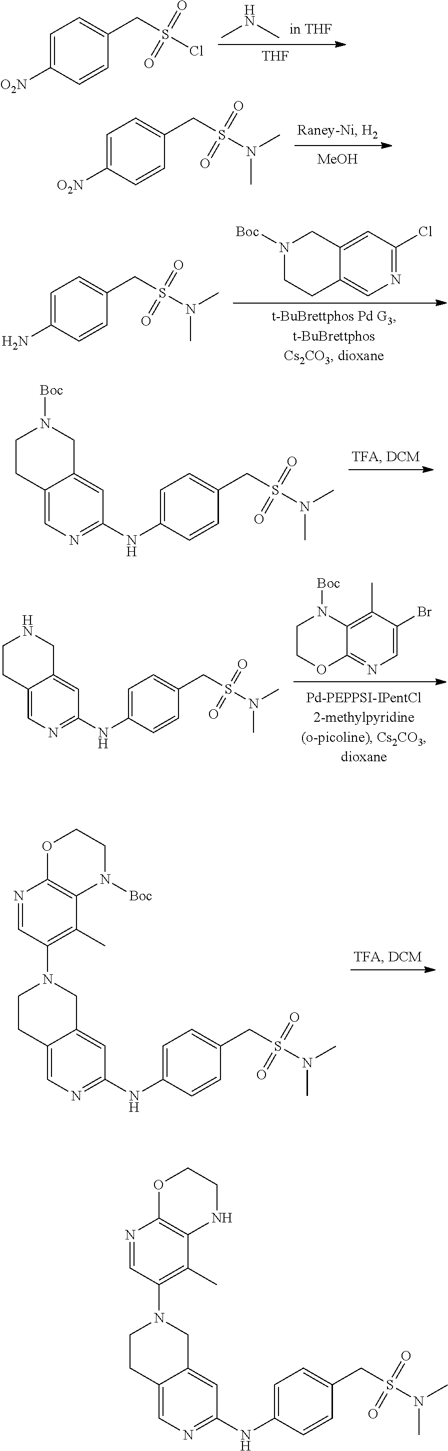

N,N-dimethyl-1-(4-nitrophenyl) methanesulfonamide: To a stirred mixture of (4-nitrophenyl)methanesulfonyl chloride (2.00 g; 8.06 mmol) in DCM (20.00 ml) was added dimethylamine (22.00 ml) and TEA (3.54 ml; 24.19 mmol) in portions at 25° C. under nitrogen atmosphere. The resulting mixture was stirred for 4 h at 25° C. under nitrogen atmosphere. The resulting mixture was extracted with EA. The combined organic layers were washed with brine, dried over anhydrous MgSO₄. After filtration, the filtrate was concentrated under reduced pressure to afford N,N-dimethyl-1-(4-nitrophenyl)methanesulfonamide (2.50 g; 92.00%) as a yellow solid.

1-(4-aminophenyl)-N,N-dimethylmethanesulfonamide: To a solution of N,N-dimethyl-1-(4-nitrophenyl)methanesulfonamide (2.00 g; 8.19 mmol) in MeOH (20.00 ml) was added Raney-Ni (1403.04 mg; 1.64 mmol) at room temperature. The mixture stirring at room temperature for 2 h under H₂ atmosphere. The resulting mixture was filtered, the filtered cake was washed with MeOH, the filtrate was concentrated under reduced pressure to afford 1-(4-aminophenyl)-N, N-dimethylmethanesulfonamide (1.70 g; Crude Product) as a light yellow solid.

tert-butyl 7-({4-[(dimethylsulfamoyl)methyl]phenyl}amino)-1,2,3,4-tetrahydro-2,6-naphthyridine-2-carboxylate: To a stirred mixture of tert-butyl 7-chloro-1,2,3,4-tetrahydro-2,6-naphthyridine-2-carboxylate (400.00 mg; 1.41 mmol) and 1-(4-aminophenyl)-N,N-dimethylmethanesulfonamide (396.73 mg; 1.70 mmol) in DMF (12.00 ml) was added t-BuBrettPhos Pd G₃ (153.00 mg; 0.17 mmol) t-BuBrettPhos (173.00 mg; 0.34 mmol) and Cs₂CO₃ (1396.10 mg; 4.24 mmol) in portions at 25° C. under nitrogen atmosphere. The resulting mixture was stirred for 6 h at 90° C. under nitrogen atmosphere. The resulting mixture was extracted with EA. The combined organic layers were washed with brine, dried over anhydrous MgSO₄. After filtration, the filtrate was concentrated under reduced pressure. The residue was purified by silica gel column chromatography, eluted with EA:PE=60:40 to afford tert-butyl 7-({4-[(dimethylsulfamoyl) methyl]phenyl}amino)-1,2,3,4-tetrahydro-2,6-naphthyridine-2-carboxylate (600.00 mg; 95.0%) as a yellow solid.

N, N-dimethyl-1-{4-[(5,6,7,8-tetrahydro-2,6-naphthyridin-3-yl) amino]phenyl}methanesulfonamide: To a stirred mixture of tert-butyl 7-({4-[(dimethylsulfamoyl) methyl]phenyl}amino)-1,2,3,4-tetrahydro-2,6-naphthyridine-2-carboxylate (240.00 mg; 0.54 mmol) in Dichloromethane (8.00 ml) was added TFA (2.00 ml) in portions at 25° C. under nitrogen atmosphere. The resulting mixture was stirred for 2 h at 25° C. under nitrogen atmosphere. The resulting mixture was concentrated under reduced pressure. The residue was purified by silica gel column chromatography, eluted with ACN: H₂O=45:55 to afford N,N-dimethyl-1-{4-[(5,6,7,8-tetrahydro-2,6-naphthyridin-3-yl)amino]phenyl}methanesulfonamide (150.00 mg; 80.65%) as a yellow solid.

tert-butyl 7-[7-({4-[(dimethylsulfamoyl)methyl]phenyl}amino)-1,2,3,4-tetrahydro-2,6-naphthyridin-2-yl]-8-methyl-1H,2H,3H-pyrido[2,3-b][1,4]oxazine-1-carboxylate: To a stirred mixture of N,N-dimethyl-1-{4-[(5,6,7,8-tetrahydro-2,6-naphthyridin-3-yl)amino]phenyl}methanesulfonamide (200.00 mg; 0.57 mmol) and tert-butyl 7-bromo-8-methyl-1H,2H,3H-pyrido[2,3-b][1,4]oxazine-1-carboxylate (282.78 mg; 0.86 mmol) in 1,4-dioxane (10.00 ml) was added Pd-PEPPSI-IPentCl 2-methylpyridine (o-picoline) (59.00 mg; 0.07 mmol) and Cs₂CO₃ (589.22 mg; 1.72 mmol) in portions at 25° C. under nitrogen atmosphere. The resulting mixture was stirred for 2 h at 90° C. under nitrogen atmosphere. The resulting mixture was extracted with EA. The combined organic layers were washed with brine, dried over anhydrous MgSO₄. After filtration, the filtrate was concentrated under reduced pressure. The residue was purified by silica gel column chromatography, eluted with ACN:H₂O=1:1 to afford tert-butyl 7-[7-({4-[(dimethylsulfamoyl)methyl]phenyl}amino)-1,2,3,4-tetrahydro-2,6-naphthyridin-2-yl]-8-methyl-1H,2H,3H-pyrido[2,3-b][1,4]oxazine-1-carboxylate (60.00 mg; 17.6%) as a yellow solid.

N,N-dimethyl-1-{4-[(6-{8-methyl-1H,2H,3H-pyrido[2,3-b][1,4]oxazin-7-yl}-5,6,7,8-tetrahydro-2,6-naphthyridin-3-yl)amino]phenyl}methanesulfonamide: To a stirred mixture of tert-butyl 7-[7-({4-[(dimethylsulfamoyl)methyl]phenyl}amino)-1,2,3,4-tetrahydro-2,6-naphthyridin-2-yl]-8-methyl-1H,2H,3H-pyrido[2,3-b][1,4]oxazine-1-carboxylate (60.00 mg; 0.10 mmol) in DCM (4.00 ml) was added TFA (1.00 ml) in portions The resulting mixture was stirred for 2 h at 25° C. under nitrogen atmosphere. The resulting mixture was concentrated under vacuum. The crude product was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 5% to 25% in 10 min; Wavelength: 254/220 nm; RT1 (min): 8.47) to afford N,N-dimethyl-1-{4-[(6-{8-methyl-1H,2H,3H-pyrido[2,3-b][1,4]oxazin-7-yl}-5,6,7,8-tetrahydro-2,6-naphthyridin-3-yl)amino]phenyl}methanesulfonamide (13.00 mg; 0.02 mmol; 24.8%).

HPLC: 95.1% purity, RT=3.24 min. MS: m/z=495.15 [M+H]⁺. ¹H NMR (400 MHz, DMSO-d6) 9.02 (s, 1H), 8.02 (s, 1H), 7.62 (dd, J=8.6, 3.1 Hz, 2H), 7.27 (d, J=8.6 Hz, 3H), 6.64 (s, 1H), 5.60 (s, 1H), 4.30 (s, 2H), 4.21 (dd, J=5.2, 3.6 Hz, 2H), 4.01 (s, 2H), 3.31 (d, J=8.9 Hz, 2H), 3.10 (t, J=5.8 Hz, 2H), 2.84 (t, J=5.7 Hz, 2H), 2.71 (s, 6H), 2.05 (s, 3H).

### Example 151: Synthesis of Compound 1-(3,3-difluoroazetidin-1-yl)-2-{4-[(7-{8-methyl-1H,2H,3H-pyrido[2,3-b][1,4]oxazin-7-yl}-5H,6H,7H,8H-pyrido[3,4-d]pyrimidin-2-yl) amino]phenyl}ethan-1-one

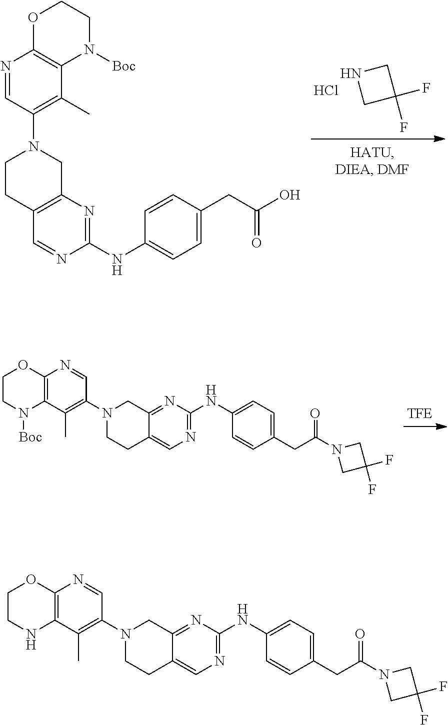 [SMILES: Cc1c(N2CCc3cnc(Nc4ccc(CC(=O)O)cc4)nc3C2)cnc2c1N(C(=O)OC(C)(C)C)CCO2 ; Cl ; FC1(F)CNC1 ; Cc1c(N2CCc3cnc(Nc4ccc(CC(=O)N5CC(F)(F)C5)cc4)nc3C2)cnc2c1N(C(=O)OC(C)(C)C)CCO2 ; Cc1c(N2CCc3cnc(Nc4ccc(CC(=O)N5CC(F)(F)C5)cc4)nc3C2)cnc2c1NCCO2]

tert-butyl 7-[2-({4-[2-(3,3-difluoroazetidin-1-yl)-2-oxoethyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate: To a stirred mixture of 3,3-difluoroazetidine hydrochloride (84.62 mg; 0.62 mmol) and DIEA (0.30 ml; 1.65 mmol) in DMF (8.00 ml) was added 2-{4-[(7-{1-[(tert-butoxy)carbonyl]-8-methyl-1H,2H,3H-pyrido[2,3-b][1,4]oxazin-7-yl}-5H,6H,7H,8H-pyrido[3,4-d]pyrimidin-2-yl)amino]phenyl}acetic acid (230.00 mg; 0.41 mmol) and HATU (331.17 mg; 0.83 mmol) in portions at 25° C. under nitrogen atmosphere. The resulting mixture was stirred for 2 h at 25° C. under nitrogen atmosphere. The resulting mixture was extracted with EA. The combined organic layers were washed with brine, dried over anhydrous MgSO₄. After filtration, the filtrate was concentrated under reduced pressure. The residue was purified by silica gel column chromatography, eluted with EA:PE=30:70 to afford tert-butyl 7-[2-({4-[2-(3,3-difluoroazetidin-1-yl)-2-oxoethyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate (220.00 mg; 80.1%) as a bright yellow oil.

1-(3,3-difluoroazetidin-1-yl)-2-{4-[(7-{8-methyl-1H,2H,3H-pyrido[2,3-b][1,4]oxazin-7-yl}-5H,6H,7H,8H-pyrido[3,4-d]pyrimidin-2-yl) amino]phenyl}ethan-1-one: A mixture of tert-butyl 7-[2-({4-[2-(3,3-difluoroazetidin-1-yl)-2-oxoethyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate (140.00 mg; 0.21 mmol) in TFE (10.00 ml) was stirred for 4 h at 120° C. under nitrogen atmosphere. The resulting mixture was concentrated under vacuum. The crude product was purified by Prep-HPLC with the following conditions (Column: XBridge Prep OBD C18 Column, 30*150 mm, 5 μm; Mobile Phase A: 10 mmol NH₄HCO₃+0.05% NH₄OH, Mobile Phase B: ACN; Flow rate: 60 mL/min; Gradient: 20% to 50% in 9 min; Wavelength: 254 nm; RT1 (min): 7) to afford 1-(3,3-difluoroazetidin-1-yl)-2-{4-[(7-{8-methyl-1H,2H,3H-pyrido[2,3-b][1,4]oxazin-7-yl}-5H,6H,7H,8H-pyrido[3,4-d]pyrimidin-2-yl) amino]phenyl}ethan-1-one (29.10 mg; 26.7%) as a light orange solid.

HPLC: 98.29% purity, RT=4067 min. MS: m/z=508.20[M+H]⁺. ¹H NMR (400 MHz, DMSO-d6) 9.50 (s, 1H), 8.33 (s, 1H), 7.72-7.64 (m, 2H), 7.29 (s, 1H), 7.16-7.08 (m, 2H), 5.55 (d, J=2.6 Hz, 1H), 4.63 (t, J=12.5 Hz, 2H), 4.28 (t, J=12.7 Hz, 2H), 4.19 (t, J=4.4 Hz, 2H), 3.96 (s, 2H), 3.46 (s, 2H), 3.33-3.27 (m, 2H), 3.12 (t, J=5.6 Hz, 2H), 2.79 (t, J=5.6 Hz, 2H), 2.04 (s, 3H).

### Example 152: Synthesis of Compound 5,5-difluoro-N-[4-(methanesulfonylmethyl) phenyl]-7-{8-methyl-1H,2H,3H-pyrido[2,3-b][1,4]oxazin-7-yl}-5H,6H,7H,8H-pyrido[3,4-d]pyrimidin-2-amine

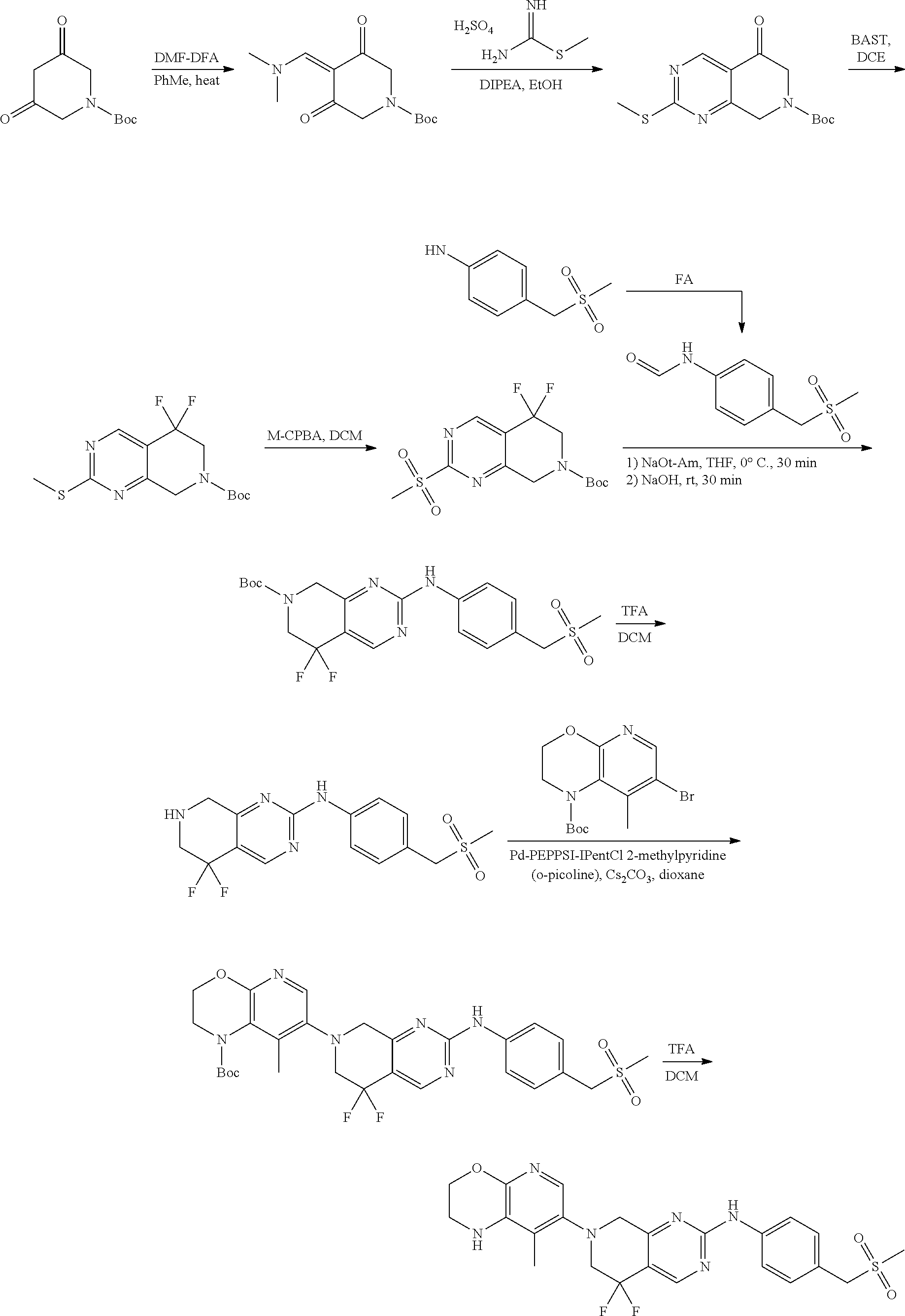

tert-butyl 4-[(dimethylamino)methylidene]-3,5-dioxopiperidine-1-carboxylate: To a stirred solution of tert-butyl 3,5-dioxopiperidine-1-carboxylate (25.00 g; 117.24 mmo) in Toluene (250.00 ml) was added N,N-Dimethylformamide dimethyl acetal (23.13 ml; 164.14 mmol) in portions at room temperature under N₂ atmosphere. The resulting mixture was stirred for 30 min at 80° C., then at 50° C. for 1 h under N₂ atmosphere. The resulting mixture was concentrated under reduced pressure to afford tert-butyl 4-[(dimethylamino)methylidene]-3,5-dioxopiperidine-1-carboxylate (30.00 g; 110.43 mmol; Crude Product) as a yellow solid.

tert-butyl 2-(methylsulfanyl)-5-oxo-5H,6H,7H,8H-pyrido[3,4-d]pyrimidine-7-carboxylate: To a solution of bis((methylsulfanyl) methanimidamide); sulfuric acid (39.32 g; 134.17 mmol) in EtOH (500.00 ml) were added DIEA (30.42 g; 223.62 mmol). The resulting mixture was stirred for 30 min at 80° C. and a solution of tert-butyl 4-[(dimethylamino)methylidene]-3,5-dioxopiperidine-1-carboxylate (30.00 g; 111.81 mmol) was then added and heating at 80° C. for 3 h. The resulting mixture was concentrated under reduced pressure. The resulting mixture was diluted with NaHCO₃(aq) (500 mL) and extracted with EtOAc. The combined organic layers were washed with brine (500 mL), dried over anhydrous Na₂SO₄. The resulting mixture was concentrated under reduced pressure. The residue was purified by silica gel column, eluted with 15% EtOAc in PE to afford tert-butyl 2-(methylsulfanyl)-5-oxo-5H,6H,7H,8H-pyrido[3,4-d]pyrimidine-7-carboxylate (7.10 g; 21.5%) as a yellow solid.

tert-butyl 5,5-difluoro-2-(methylsulfanyl)-5H,6H,7H,8H-pyrido[3,4-d]pyrimidine-7-carboxylate: To a stirred mixture of tert-butyl 2-(methylsulfanyl)-5-oxo-5H,6H,7H,8H-pyrido[3,4-d]pyrimidine-7-carboxylate (3.00 g; 10.16 mmol) in DCE (15.00 ml) was added BAST (11.83 g; 50.79 mmol) dropwise at 25° C. The resulting mixture was stirred for 2 h at 50° C. under nitrogen atmosphere. The reaction was quenched with NaHCO₃ at room temperature. The resulting mixture was extracted with EA. The combined organic layers were washed with brine, dried over anhydrous MgSO₄. After filtration, the filtrate was concentrated under reduced pressure. The residue was purified by silica gel column chromatography, eluted with PE: EA=1:4 to afford tert-butyl 5,5-difluoro-2-(methylsulfanyl)-5H,6H,7H,8H-pyrido[3,4-d]pyrimidine-7-carboxylate (1.80 g; 55.8%) as a yellow solid.

tert-butyl 5,5-difluoro-2-methanesulfonyl-5H,6H,7H,8H-pyrido[3,4-d]pyrimidine-7-carboxylate: To a stirred mixture of tert-butyl 5,5-difluoro-2-(methylsulfanyl)-5H,6H,7H,8H-pyrido[3,4-d]pyrimidine-7-carboxylate (1.80 g; 5.67 mmol) in DCM (40.00 ml) was added m-CPBA (2.58 g; 14.18 mmol) in portions at 25° C. The resulting mixture was stirred for 16 h at 25° C. under nitrogen atmosphere. The reaction was quenched with NaHCO₃ at room temperature. The resulting mixture was extracted with EA. The combined organic layers were washed with brine, dried over anhydrous MgSO₄. After filtration, the filtrate was concentrated under reduced pressure. The residue was purified by silica gel column chromatography, eluted with PE:EA=38:62 to afford tert-butyl 5,5-difluoro-2-methanesulfonyl-5H,6H,7H,8H-pyrido[3,4-d]pyrimidine-7-carboxylate (900.00 mg; 45.4%) as a yellow solid.

N-[4-(methanesulfonylmethyl)phenyl] formamide: To a stirred mixture of 4-(methanesulfonylmethyl) aniline (10.70 g; 56.92 mmol) in formic acid (130.00 ml) at 25° C. The resulting mixture was stirred for 3 h at 80° C. under nitrogen atmosphere. The resulting mixture was concentrated under reduced pressure. The resulting in N-[4-(methanesulfonylmethyl)phenyl] formamide (12.00 g; Crude Product) as a yellow solid.

tert-butyl 5,5-difluoro-2-{[4-(methanesulfonylmethyl)phenylamino}-5H,6H,7H,8H-pyrid [3,4-dpyrimidine-7-carboxylate: To a stirred mixture of N-[4-(methanesulfonylmethyl)phenyl] formamide (134.00 mg; 0.63 mmol) in THF (5.00 ml) was added Sodium tert-pentoxide (73.00 mg; 0.63 mmol) at 0° C. The resulting mixture was stirred for 0.5 h at 0° C. Then tert-butyl 5,5-difluoro-2-methanesulfonyl-5H,6H,7H,8H-pyrido[3,4-d]pyrimidine-7-carboxylate (110.00 mg; 0.31 mmol) was added and the mixture was stirred at for 30 min at 0° C. NaOH (0.50 ml; 0.50 mmol) was added and the mixture was stirred at for 30 min at room temperature. Diluted with EA, the filter cake was washed with EA. The filtrate was concentrated under reduced pressure. The residue was purified by silica gel column, eluted with EA:PE=1:1 to afford tert-butyl 5,5-difluoro-2-{[4-(methanesulfonylmethyl)phenyl]amino}-5H,6H,7H,8H-pyrido[3,4-d]pyrimidine-7-carboxylate (500.00 mg; 1.10 mmol; 42.7%; yellow solid).

5,5-difluoro-N-[4-(methanesulfonylmethyl)phenyl]-5H,6H,7H,8H-pyrido[3,4-d]pyrimidin-2-amine: To a stirred solution of tert-butyl 5,5-difluoro-2-{[4-(methanesulfonylmethyl)phenyl]amino}-5H,6H,7H,8H-pyrido[3,4-d]pyrimidine-7-carboxylate (500.00 mg; 1.10 mmol) in DCM (6.00 ml) was added TFA (2.00 ml) dropwise at room temperature. The resulting mixture was stirred for 2 h at room temperature. Column: XBridge Prep OBD C18 Column, 30*150 mm, 5 μm; Mobile Phase A: Water (10 mmol/L NH₄HCO₃+0.05% NH₄OH), Mobile Phase B: ACN; Flow rate: 60 m/min; Gradient: 16% B to 45% B in 9 min; Wavelength: 254 nm; RT1 (min): 7. This resulted in 5,5-difluoro-N-[4-(methanesulfonylmethyl) phenyl]-5H,6H,7H,8H-pyrido[3,4-d]pyrimidin-2-amine (200.00 mg; 39.3%) as a yellow solid.

tert-butyl 7-(5,5-difluoro-2-{[4-(methanesulfonylmethyl) phenyl]amino}-5H,6H,7H,8H-pyrido[3,4-d]pyrimidin-7-yl)-8-methyl-1H,2H,3H-pyrido[2,3-b][1,4]oxazine-1-carboxylate: A solution of 5,5-difluoro-N-[4-(methanesulfonylmethyl)phenyl]-5H,6H,7H,8H-pyrido[3,4-d]pyrimidin-2-amine (150.00 mg; 0.32 mmol), tert-butyl 7-bromo-8-methyl-1H,2H,3H-pyrido[2,3-b]oxazine-1-carboxylate (238.00 mg; 0.65 mmol), Pd-PEPPSI-IPentCl 2-methylpyridine (o-picoline) (29.00 mg; 0.03 mmol) and Cs₂CO₃ (223.00 mg; 0.65 mmol) in 1,4-dioxane (5.00 ml) was stirred for 2 h at 100° C. under nitrogen atmosphere. The resulting mixture was extracted with EA. The combined organic layers were washed with brine, dried over anhydrous MgSO₄. After filtration, the filtrate was concentrated under reduced pressure. The residue was purified by silica gel column chromatography, eluted with PE:EA=4:1 to afford tert-butyl 7-(5,5-difluoro-2-{[4-(methanesulfonylmethyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (100.00 mg; 51.1%) as a yellow solid.

5,5-difluoro-N-[4-(methanesulfonylmethyl)phenyl]-7-{8-methyl-1H,2H,3H-pyrido[2,3-b][1,4]oxazin-7-yl}-5H,6H,7H,8H-pyrido[3,4-d]pyrimidin-2-amine: A solution of tert-butyl 7-[4(methanesulfonylmethyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (70.00 mg; 0.12 mmol) in 4 N HCl in EA (3.00 ml) was stirred for 2 h at room temperature. The residue was purified by Prep-HPLC with the following condition Column: XBridge Shield RP18 OBD Column, 30*150 mm, 5 μm; Mobile Phase A: Water (10 mmol/L NH₄HCO₃+0.05% NH₄OH), Mobile Phase B: ACN; Flow rate: 60 mL/min; Gradient: 24% to 54% in 9 min; Wavelength: 254 nm; RT1 (min): 7. This resulted in 5,5-difluoro-N-[4-(methanesulfonylmethyl) phenyl]-7-{8-methyl-1H,2H,3H-pyrido[2,3-b][1,4]oxazin-7-yl}-5H,6H,7H,8H-pyrido[3,4-d]pyrimidin-2-amine (13.70 mg; 23.2%) as a yellow solid.

HPLC: 99.02% purity, RT=3.14 min. MS: m/z=503.20 [M+H]⁺. ¹H NMR (400 MHz, DMSO-d6): ¹H NMR (300 MHz, DMSO-d6) 10.21 (s, 1H), 8.85 (s, 1H), 7.78 (d, J=8.3 Hz, 2H), 7.45-7.30 (m, 3H), 5.60 (s, 1H), 4.41 (s, 2H), 4.15 (d, J=32.8 Hz, 4H), 3.59 (t, J=11.4 Hz, 2H), 3.34 (s, 2H), 2.88 (s, 3H), 2.05 (s, 3H).

### Example 153: Synthesis of Compound 6-(2-methoxyethyl)-2-[(6-{8-methyl-1H,2H,3H-pyrido[2,3-b][1,4]oxazin-7-yl}-5,6,7,8-tetrahydro-2,6-naphthyridin-3-yl) amino]-4H,5H,6H,7H,8H-pyrazolo[1,5-d][1,4]diazepin-7-one

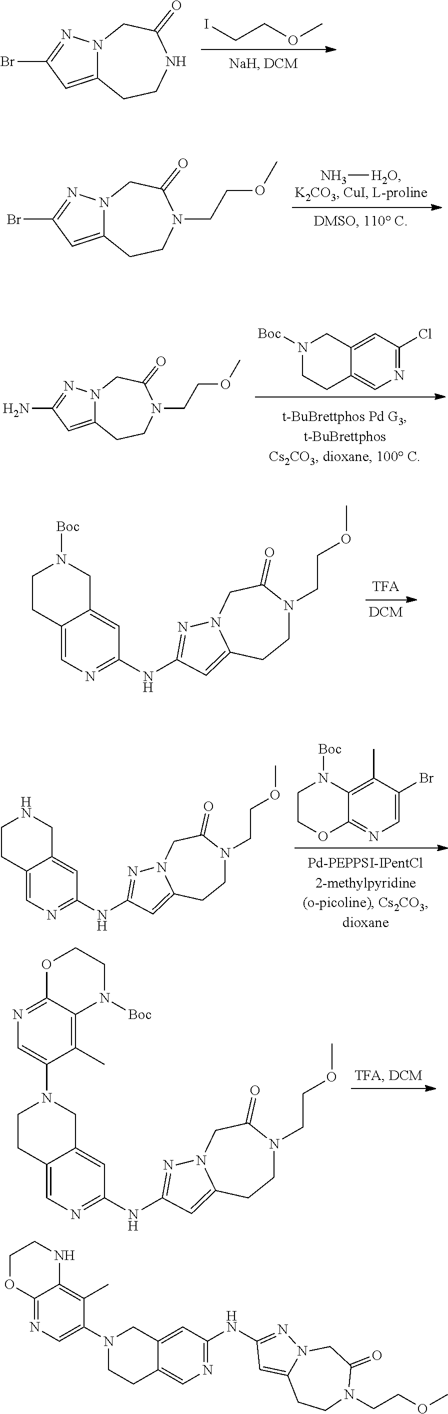

2-bromo-6-(2-methoxyethyl)-4H,5H,6H,7H,8H-pyrazolo[1,5-d][1,4]diazepin-7-one: To a stirred solution of 2-bromo-4H,5H,6H,7H,8H-pyrazolo[1,5-d][1,4]diazepin-7-one (3.00 g; 11.66 mmol) in DMF (30.00 ml) was added NaH (1.86 g; 46.62 mmol) in portions at 0° C. under N₂ atmosphere. The resulting mixture was stirred for 30 min at 0° C. under N₂ atmosphere. A solution of was added 1-iodo-2-methoxyethane (6.85 g; 34.97 mmol) in portions at 0° C. under N₂ atmosphere. The resulting mixture was stirred for 30 min at room temperature under N₂ atmosphere. The resulting mixture was diluted with water, extracted with EtOAc. The combined organic layers were washed with brine, dried over anhydrous Na₂SO₄. After filtration, the resulting mixture was concentrated under reduced pressure. The residue was purified by C18 flash column eluted with 30% ACN in Water to afford 2-bromo-6-(2-methoxyethyl)-4H,5H,6H,7H,8H-pyrazolo[1,5-d][1,4]diazepin-7-one (1.05 g; 31.3%) as an off-white solid.

2-amino-6-(2-methoxyethyl)-4H,5H,6H,7H,8H-pyrazolo[1,5-d]diazepin-7-one: To a stirred solution of 2-bromo-6-(2-methoxyethyl)-4H,5H,6H,7H,8H-pyrazolo[1,5-d][1,4]diazepin-7-one (1.0 g; 3.48 mmol) and ammonium hydroxide solution (2.00 ml; 1518.26 mmol) in DMSO (10.00 ml) was added CuI (139.00 mg; 0.69 mmol), L-proline (168.00 mg; 1.39 mmol) and K₂CO₃ (1.00 g; 6.87 mmol). The resulting mixture was stirred for overnight at 90° C. under nitrogen atmosphere. The residue was purified by C18 flash column eluted with 30% ACN in water to afford 2-amino-6-(2-methoxyethyl)-4H,5H,6H,7H,8H-pyrazolo[1,5-d][1,4]diazepin-7-one (700.00 mg; 89.9%) as a yellow solid.

tert-butyl 7-{[6-(2-methoxyethyl)-7-oxo-4H,5H,6H,7H,8H-pyrazolo[1,5-d][1,4]diazepin-2-yl]amino}-1,2,3,4-tetrahydro-2,6-naphthyridine-2-carboxylate: To a stirred solution of 2-amino-6-(2-methoxyethyl)-4H,5H,6H,7H,8H-pyrazolo[1,5-d][1,4]diazepin-7-one (410.00 mg; 1.72 mmol) and tert-butyl 7-chloro-1,2,3,4-tetrahydro-2,6-naphthyridine-2-carboxylate (526.00 mg; 1.92 mmol) in 1,4-dioxane (10.00 ml) were added t-BuBrettPhos Pd G₃ (153.00 mg; 0.17 mmol) t-BuBrettPhos (173.00 mg; 0.34 mmol) and Cs₂CO₃ (1.80 g; 5.25 mmol) in portions at room temperature under N₂ atmosphere. The resulting mixture was stirred for overnight at 100° C. under N₂ atmosphere. The resulting mixture was diluted with water, extracted with EtOAc. The combined organic layers were washed with brine, dried over anhydrous Na₂SO₄. The resulting mixture was concentrated under reduced pressure. The residue was purified by silica gel column, eluted with 100% EtOAc to afford tert-butyl 7-{[6-(2-methoxyethyl)-7-oxo-4H,5H,6H,7H,8H-pyrazolo[1,5-d][1,4]diazepin-2-yl]amino}-1,2,3,4-tetrahydro-2,6-naphthyridine-2-carboxylate (260.00 mg; 33.0%) as an off-white solid.

6-(2-methoxyethyl)-2-[(5,6,7,8-tetrahydro-2,6-naphthyridin-3-yl) amino]-4H,5H,6H,7H,8H-pyrazolo[1,5-d][1,4]diazepin-7-one: To a stirred solution of tert-butyl 7-{[6-(2-methoxyethyl)-7-oxo-4H,5H,6H,7H,8H-pyrazolo[1,5-d][1,4]diazepin-2-yl]amino}-1,2,3,4-tetrahydro-2,6-naphthyridine-2-carboxylate (240.00 mg; 0.53 mmol) in DCM (6.00 ml) was added TFA (2.00 ml) dropwise at room temperature. The resulting mixture was stirred for 2 h at room temperature. The resulting mixture was concentrated under reduced pressure. The residue was purified by silica gel column chromatography, eluted with PE:EA=1:1 to afford 6-(2-methoxyethyl)-2-[(5,6,7,8-tetrahydro-2,6-naphthyridin-3-yl) amino]-4H,5H,6H,7H,8H-pyrazolo[1,5-d][1,4]diazepin-7-one (160.00 mg; 85.4%) as a yellow solid.

tert-butyl 7-(7-{[6-(2-methoxyethyl)-7-oxo-4H,5H,6H,7H,8H-pyrazolo[1,5-d][1,4]diazepin-2-yl]amino}-1,2,3,4-tetrahydro-2,6-naphthyridin-2-yl)-8-methyl-1H,2H,3H-pyrido[2,3-b][1,4]oxazine-1-carboxylate: A solution of 6-(2-methoxyethyl)-2-[(5,6,7,8-tetrahydro-2,6-naphthyridin-3-yl) amino]-4H,5H,6H,7H,8H-pyrazolo[1,5-d][1,4]diazepin-7-one (140.00 mg; 0.39 mmol) and tert-butyl 7-bromo-8-methyl-1H,2H,3H-pyrido[2,3-b][1,4]oxazine-1-carboxylate (288.00 mg; 0.79 mmol) and Pd-PEPPSI-IPentCl 2-methylpyridine (o-picoline) (35.00 mg; 0.04 mmol) and Cs₂CO₃ (270.00 mg; 0.79 mmol) in 1,4-dioxane (5.00 ml) was stirred for 2 h at 100° C. under nitrogen atmosphere. The resulting mixture was extracted with EA (3×100 mL). The combined organic layers were washed with brine, dried over anhydrous MgSO₄. After filtration, the filtrate was concentrated under reduced pressure. The residue was purified by silica gel column chromatography, eluted with PE: EA:4:1 to afford tert-butyl 7-(7-{[6-(2-methoxyethyl)-7-oxo-4H,5H,6H,7H,8H-pyrazolo[1,5-d][1,4]diazepin-2-yl]amino}-1,2,3,4-tetrahydro-2,6-naphthyridin-2-yl)-8-methyl-1H,2H,3H-pyrido[2,3-b][1,4]oxazine-1-carboxylate (130.00 mg; 51.1%) as a yellow solid.

6-(2-methoxyethyl)-2-[(6-{8-methyl-1H,2H,3H-pyrido[2,3-b][1,4]oxazin-7-yl}-5,6,7,8-tetrahydro-2,6-naphthyridin-3-yl)amino]-4H,5H,6H,7H,8H-pyrazolo[1,5-d][1,4]diazepin-7-one: To a stirred solution of tert-butyl 7-(7-{[6-(2-methoxyethyl)-7-oxo-4H,5H,6H,7H,8H-pyrazolo[1,5-d][1,4]diazepin-2-yl]amino}-1,2,3,4-tetrahydro-2,6-naphthyridin-2-yl)-8-methyl-1H,2H,3H-pyrido[2,3-b][1,4]oxazine-1-carboxylate (120.00 mg; 0.19 mmol) in DCM (3.00 ml) was added TFA (1.00 ml) dropwise at room temperature. The resulting mixture was stirred for 2 h at room temperature. Column: Xselect CSH C18 OBD Column 30*150 mm 5 μm, n; Mobile Phase A: Water (0.1% FA), Mobile Phase B: ACN; Flow rate: 60 mL/min; Gradient: 30% B to 60% B in 8 min, Wavelength: 254 nm; RT1 (min): 7. This resulted in 6-(2-methoxyethyl)-2-[(6-{8-methyl-1H,2H,3H-pyrido[2,3-b][1,4]oxazin-7-yl}-5,6,7,8-tetrahydro-2,6-naphthyridin-3-yl) amino]-4H,5H,6H,7H,8H-pyrazolo[1,5-d][1,4]diazepin-7-one (26.40 mg; 27.0%) as an off-white solid.

HPLC: 95.39% purity, RT=2.14 min. MS: m/z=505.30 [M+H]⁺. ¹H NMR (300 MHz, DMSO-d6) 8.04 (s, 1H), 7.28 (s, 1H), 7.07 (s, 1H), 6.06 (s, 1H), 5.10 (s, 2H), 4.22 (d, J=4.7 Hz, 2H), 4.14 (s, 2H), 3.89 (s, 2H), 3.56 (t, J=5.4 Hz, 2H), 3.45 (t, J=5.3 Hz, 2H), 3.32 (t, J=4.4 Hz, 2H), 3.24 (s, 3H), 3.08 (t, J=7.3 Hz, 4H), 2.89 (s, 2H), 2.05 (s, 3H).

### Example 154: Synthesis of Compound 1N-(4-{[2-(dimethylamino) ethanesulfonyl]methyl}phenyl)-7-{8-methyl-1H,2H,3H-pyrido[2,3-b][1,4]oxazin-7-yl}-5H,6H,7H,8H-pyrido[3,4-d]pyrimidin-2-amine

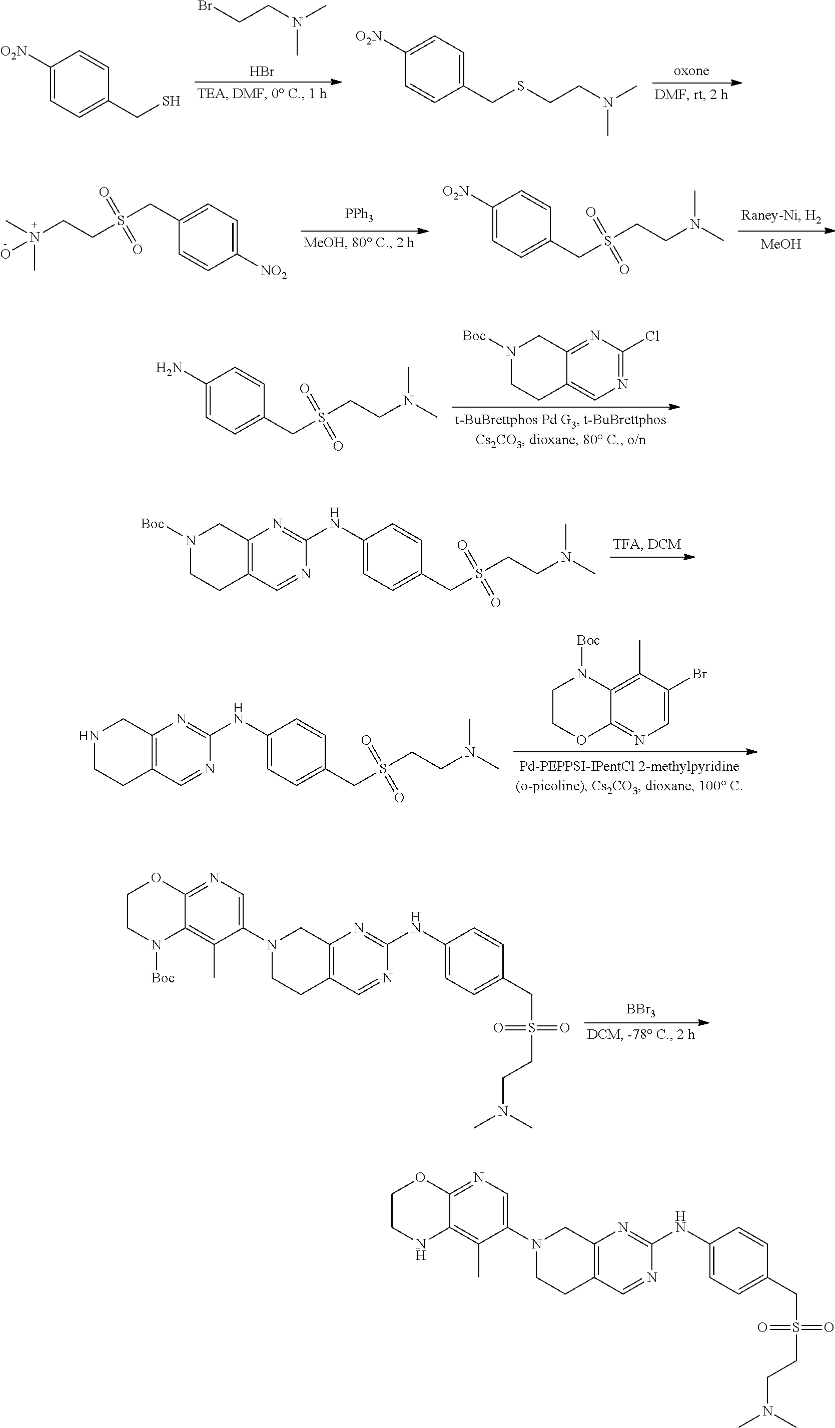

dimethyl(2-{[(4-nitrophenyl)methyl]sulfanyl}ethyl)amine: To a stirred mixture of (4-nitrophenyl)methanethiol (5.00 g; 27.72 mmol),(2-bromoethyl)dimethylamine hydrobromide (8.16 g; 33.26 mmol) in DMF (50.00 ml) was added TEA (12.17 ml) at 25° C. The resulting mixture was stirred for 1 h at 0° C. under nitrogen atmosphere. The resulting mixture was extracted with EtOAc. The combined organic layers were washed with NaCl(aq.), dried over anhydrous Na₂SO₄. After filtration, the filtrate was concentrated under reduced pressure to afford dimethyl(2-{1[(4-nitrophenyl)methyl]sulfanyl}ethyl)amine (4.20 g; 63.1%) as a yellow solid.

1-{[2-(dimethyl-oxo-5-azanyl)ethanesulfonyl]methyl}-4-nitrobenzene: To a stirred mixture of dimethyl(2-{[(4-nitrophenyl) methyl] sulfanyl}ethyl) amine (3.70 g; 15.40 mmol) in DMF (80.00 ml) was added oxone (29.89 g; 46.19 mmol) in portions at 25° C. The resulting mixture was stirred for 2 h at room temperature under nitrogen atmosphere. The resulting mixture was concentrated under reduced pressure. The resulting mixture was extracted with EtOAc (3×1000 mL). The combined organic layers were washed with brine (50 mL), dried over anhydrous Na₂SO₄. The resulting mixture was concentrated under reduced pressure. The residue was purified by silica gel column, eluted with 50% EA in PE to afford 1-{[2-(dimethyl-oxo-5-azanyl)ethanesulfonyl]methyl}-4-nitrobenzene (3.80 g; 85.6%) as a yellow solid.

dimethyl({2-[(4-nitrophenyl) methanesulfonyl]ethyl})amine: To a stirred mixture of 1-{[2-(dimethyl-oxo-5-azanyl)ethanesulfonyl]methyl}-4-nitrobenzene (3.70 g; 12.83 mmol) in MeOH (80.00 ml) was added PPh₃ (7.09 g; 25.67 mmol) at 25° C. The resulting mixture was stirred for 20 min at 80° C. under nitrogen atmosphere. The resulting mixture was extracted with EtOAc (3×100 mL). The combined organic layers were washed with brine (50 mL) and dried over anhydrous Na₂SO₄. The resulting mixture was concentrated under reduced pressure. The residue was purified by silica gel column, eluted with 50% EA in PE to afford dimethyl({2-[(4-nitrophenyl) methanesulfonyl]ethyl})amine (1.60 g; 45.8%) as a yellow solid.

4-{[2-(dimethylamino) ethanesulfonyl] methyl}aniline: To a solution of dimethyl({2-[(4-nitrophenyl) methanesulfonyl]ethyl})amine (1.60 g; 5.88 mmol) in MeOH (40.00 ml) was added Raney-Ni (795.00 mg; 8.81 mmol) in a pressure tank. The mixture was hydrogenated in hydrogen (0.27 ml; 11.90 mmol) atmosphere at room temperature for overnight. The resulting mixture was concentrated under reduced pressure. The residue was purified by silica gel column chromatography, eluted with PE/EA (1:1) to afford 4-{[2-(dimethylamino) ethanesulfonyl] methyl}aniline (1.20 g; 84.3%;) as a yellow solid.

tert-butyl 2-[(4-{[2-(dimethylamino)ethanesulfonyl]methyl}phenyl)amino]-5H,6H,7H,8H-pyrido[3,4-d]pyrimidine-7-carboxylate: To a stirred solution of 4-{[2-(dimethylamino) ethanesulfonyl] methyl}aniline (1.10 g; 4.54 mmol) and tert-butyl 7-chloro-1,2,3,4-tetrahydro-2,6-naphthyridine-2-carboxylate (1494.00 mg; 5.45 mmol) in 1,4-dioxane (25.00 ml) were added t-BuBrettPhos Pd G₃ (409.00 mg; 0.45 mmol) t-BuBrettPhos (232.00 mg; 0.45 mmol) and Cs₂CO₃ (3110.00 mg; 9.07 mmol) in portions at room temperature under N₂ atmosphere. The resulting mixture was stirred for overnight at 80° C. under N₂ atmosphere. The resulting mixture was diluted with water, extracted with EtOAc. The combined organic layers were washed with brine, dried over anhydrous Na₂SO₄. The resulting mixture was concentrated under reduced pressure. The residue was purified by silica gel column, eluted with 100% EtOAc to afford tert-butyl 2-[(4-{[2-(dimethylamino)ethanesulfonyl]methyl}phenyl)amino]-5H,6H,7H,8H-pyrido[3,4-d]pyrimidine-7-carboxylate (400.00 mg; 18.5%) as an off-white solid.

N-(4-{[2-(dimethylamino) ethanesulfonyl] methyl}phenyl)-5H,6H,7H,8H-pyrido[3,4-d]pyrimidin-2-amine: To a stirred solution of tert-butyl 2-[(4-{[2-(dimethylamino)ethanesulfonyl]methyl}phenyl)amino]-5H,6H,7H,8H-pyrido[3,4-d]pyrimidine-7-carboxylate (400.00 mg; 0.84 mmol) in DCM (9.00 ml) was added TFA (3.00 ml) dropwise at room temperature. The resulting mixture was stirred for 2 h at room temperature. The residue was purified by Prep-HPLC with the follow condition Column: YMC-Actus Triart C18 ExRS, 30*150 mm, 5 μm; Mobile Phase A: Water (10 mmol/L NH₄HCO₃+0.05% NH₄OH), Mobile Phase B: ACN; Flow rate: 60 mL/min; Gradient: 20% B to 50% B in 8 min, Wavelength: 254 nm; RT1 (min): 6.5. This resulted in N-(4-{[2-(dimethylamino)ethanesulfonyl]methyl}phenyl)-5H,6H,7H,8H-pyrido[3,4-d]pyrimidin-2-amine (260.00 mg; 82.3%) as a yellow solid.

tert-butyl 7-{2-[(4-{[2-(dimethylamino) ethanesulfonyl] methyl}phenyl) amino]-5H,6H,7H,8H-pyrido[3,4-d]pyrimidin-7-yl}-8-methyl-1H,2H,3H-pyrido[2,3-b][1,4]oxazine-1-carboxylate: A solution of N-(4-{[2-(dimethylamino) ethanesulfonyl] methyl}phenyl)-5H,6H,7H,8H-pyrido[3,4-d]pyrimidin-2-amine (240.00 mg; 0.64 mmol), tert-butyl 7-bromo-8-methyl-1H,2H,3H-pyrido[2,3-b][1,4]oxazine-1-carboxylate (281.00 mg; 0.77 mmol; 1.20 eq.), Pd-PEPPSI-IPentCl 2-methylpyridine (o-picoline) (57.00 mg; 0.06 mmol) and Cs₂CO₃ (439.00 mg; 1.28 mmol) in 1,4-dioxane (8.00 ml) was stirred for 2 h at 100° C. under nitrogen atmosphere. The resulting mixture was diluted with water and extracted with EtOAc. The combined organic layers were washed with brine, dried over anhydrous Na₂SO₄. The resulting mixture was concentrated under reduced pressure. The residue was purified by silica gel column, eluted with 20% MeOH to afford tert-butyl 7-{2-[(4-{[2-(dimethylamino) ethanesulfonyl] methyl}phenyl)amino]-5H,6H,7H,8H-pyrido[3,4-d]pyrimidin-7-yl}-8-methyl-1H,2H,3H-pyrido[2,3-b][1,4]oxazine-1-carboxylate (200.00 mg; 50.2%) as a yellow solid.

N-(4-{[2-(dimethylamino)ethanesulfonyl]methyl}phenyl)-7-{8-methyl-1H,2H,3H-pyrido[2,3-b][1,4]oxazin-7-yl}-5H,6H,7H,8H-pyrido[3,4-d]pyrimidin-2-amine: To a stirred solution of tert-butyl 7-{2-[(4-{[2-(dimethylamino) ethanesulfonyl] methyl}phenyl) amino]-5H,6H,7H,8H-pyrido[3,4-d]pyrimidin-7-yl}-8-methyl-1H,2H,3H-pyrido[2,3-b][1,4]oxazine-1-carboxylate (170.00 mg; 0.27 mmol) in DCM (5.00 ml) was added BBr₃ (1 mol/L) in DCM (0.82 ml) dropwise at −78 degree C. The resulting mixture was stirred for 2 h at −78° C. The resulting mixture was quenched with NaHCO₃(aq.) between −78° C. and −50° C. The resulting mixture was extracted with DCM. The combined organic layers was dried over anhydrous Na₂SO₄ and concentrated under reduced pressure. The residue was purified by Prep-HPLC with the following condition Column: XBridge Prep OBD C18 Column, 30*150 mm, 5 μm; Mobile Phase A: Water (10 mmol/L NH₄HCO₃+0.05% NH₄OH), Mobile Phase B: ACN; Flow rate: 60 mL/min; Gradient: 22% B to 50% B in 9 min, Wavelength: 254 nm; RT1 (min): 7. This resulted in N-(4-{[2-(dimethylamino)ethanesulfonyl]methyl}phenyl)-7-{8-methyl-1H,2H,3H-pyrido[2,3-b][1,4]oxazin-7-yl}-5H,6H,7H,8H-pyrido[3,4-d]pyrimidin-2-amine (45.10 mg; 31.0%) as a light yellow solid.

HPLC: 97.94% purity, RT=3.25 min. MS: m/z=524.2 [M+H]⁺. ¹H NMR (300 MHz, DMSO-d6) 9.64 (s, 1H), 8.35 (s, 1H), 7.77 (d, J=8.6 Hz, 2H), 7.32-7.22 (m, 3H), 5.53 (s, 1H), 4.41 (s, 2H), 4.18 (d, J=4.5 Hz, 2H), 3.97 (s, 2H), 3.30 (s, 2H), 3.22-3.09 (m, 4H), 2.80 (s, 2H), 2.63 (t, J=7.2 Hz, 2H), 2.16 (s, 6H), 2.04 (s, 3H)

### Example 155: Synthesis of Compound 1-(3-fluoroazetidin-1-yl)-2-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}ethan-1-one

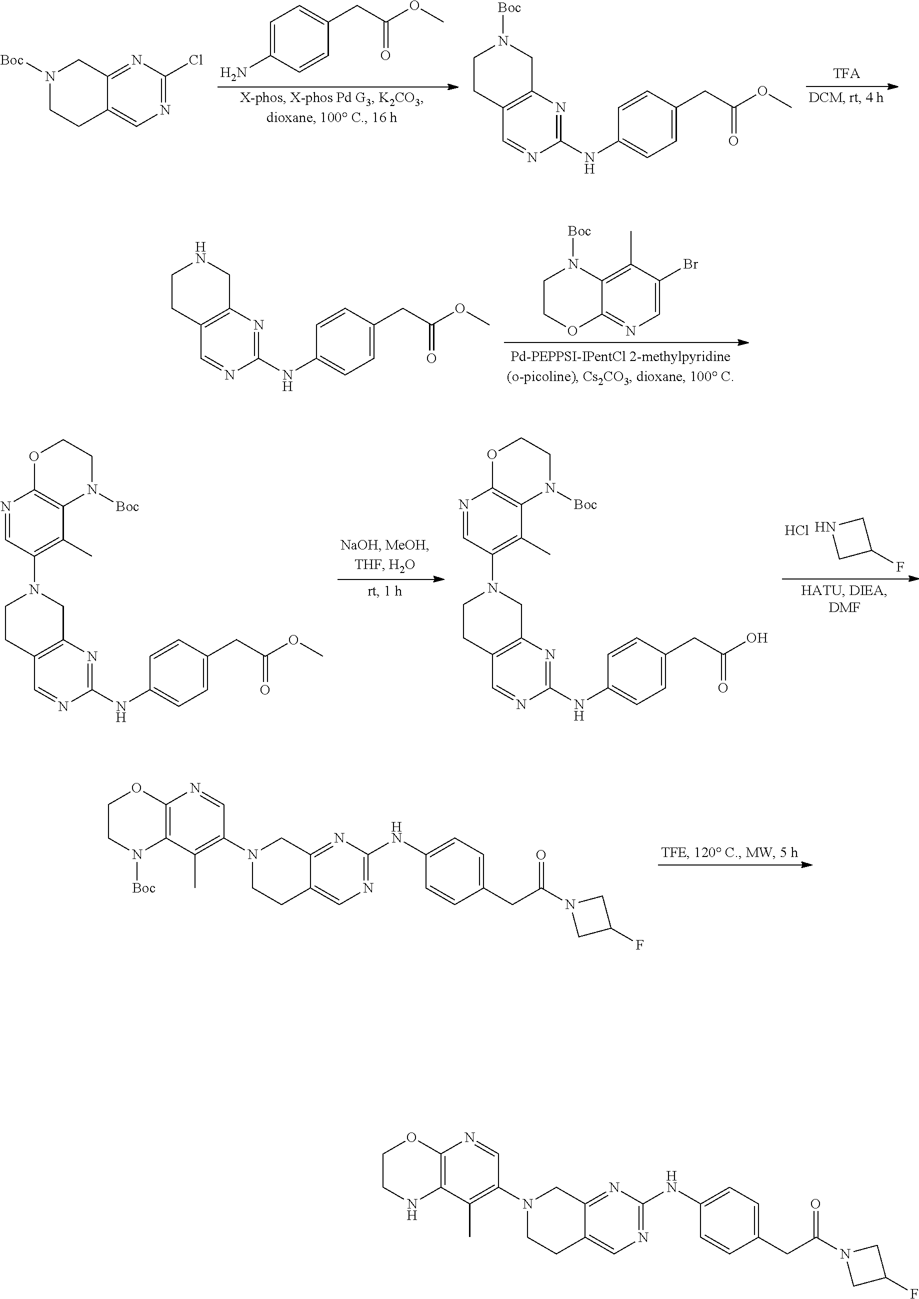

tert-butyl 2-{[4-(2-methoxy-2-oxoethyl)phenyl]amino}-5H,6H,7H,8H-pyrido[3,4-d]pyrimidine-7-carboxylate: To a stirred mixture of tert-butyl 2-chloro-5H,6H,7H,8H-pyrido[3,4-d]pyrimidine-7-carboxylate (5.00 g; 18.06 mmol) and were added X-PHOS (1.81 g; 3.61 mmol), K₂CO₃ (5.25 g; 36.12 mmol) and XPhos Pd G₃ (1.61 g; 1.81 mmol) in portions at 25° C. The resulting mixture was stirred for 2 h at 100° C. under nitrogen atmosphere. The resulting mixture was concentrated under vacuum. The residue was purified by silica gel column chromatography, eluted with DCM:MeOH=10:1 to afford tert-butyl 2-{[4-(2-methoxy-2-oxoethyl)phenyl]amino}-5H,6H,7H,8H-pyrido[3,4-d]pyrimidine-7-carboxylate (5.28 g; 70.4%) as a yellow solid.

methyl 2-[4-({5H,6H,7H,8H-pyrido[3,4-d]pyrimidin-2-yl}amino) phenyl] acetate: To a stirred mixture of tert-butyl 2-{[4-(2-methoxy-2-oxoethyl)phenyl]amino}-5H,6H,7H,8H-pyrido[3,4-d]pyrimidine-7-carboxylate (5.18 g; 13.00 mmol) in DCM (50.00 ml) was added TFA (15.00 ml; 201.94 mmol) in portions at 25° C. The resulting mixture was stirred for 2 h at 25° C. under nitrogen atmosphere. The resulting mixture was concentrated under reduced pressure and purified by silica gel column chromatography, eluted with CH₂Cl₂/MeOH (1:1) to afford methyl 2-[4-({5H,6H,7H,8H-pyrido[3,4-d]pyrimidin-2-yl}amino)phenyl]acetate (3.10 g; 78.6%) as a pink solid.

tert-butyl 7-(2-{[4-(2-methoxy-2-oxoethyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate: To a stirred mixture of tert-butyl 7-bromo-8-methyl-1H,2H,3H-pyrido[2,3-b][1,4]oxazine-1-carboxylate (4.20 g; 12.76 mmol) and methyl 2-[4-({5H,6H,7H,8H-pyrido[3,4-d]pyrimidin-2-yl}amino)phenyl] acetate (3.50 g; 11.50 mmol) in Dioxane-1,4 (80.00 ml) were added Pd-PEPPSI-IPentCl 2-methylpyridine (o-picoline) (0.30 g; 0.33 mmol) and Cs₂CO₃ (7.89 g; 22.99 mmol) at room temperature. The mixture was stirred for 12 h at 100° C. under argon atmosphere. The resulting mixture was concentrated under reduced pressure and purified by silica gel column chromatography, eluted with PE/EtOAc (1:1) to afford tert-butyl 7-(2-{[4-(2-methoxy-2-oxoethyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (4.31 g; 51.9%) as a yellow oil.

2-{4-[(7-{1-[(tert-butoxy)carbonyl]-8-methyl-1H,2H,3H-pyrido[2,3-b][1,4]oxazin-7-yl}-5H,6H,7H,8H-pyrido[3,4-d]pyrimidin-2-yl) amino]phenyl}acetic acid: To a stirred solution of tert-butyl 7-(2-{[4-(2-methoxy-2-oxoethyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (4.31 g; 7.13 mmol) in THF (40.00 ml), H₂O (20.00 ml) and MeOH (40.00 ml) was added NaOH (1.30 g; 30.88 mmol) in portions at room temperature. The mixture was stirred for 3 h at 50° C. The solvent was removed under vacuum, and 20 ml water was added in the residue, and adjust PH=6-7 with Conc. HCl, the solid was collected by filtration and dried under vacuum. This resulted in 2-{4-[(7-{1-[(tert-butoxy)carbonyl]-8-methyl-1H,2H,3H-pyrido[2,3-b][1,4]oxazin-7-yl}-5H,6H,7H,8H-pyrido[3,4-d]pyrimidin-2-yl) amino]phenyl}acetic acid (3.80 g; 95.9%) as a yellow solid.

tert-butyl 7-[2-({4-[2-(3-fluoroazetidin-1-yl)-2-oxoethyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate: To a stirred mixture of 3-fluoroazetidine hydrochloride (73.00 mg; 0.62 mmol) and DIEA (0.30 ml; 1.65 mmol) in DMF (5.00 ml) was added 2-{4-[(7-{1-[(tert-butoxy) carbonyl]-8-methyl-1H,2H,3H-pyrido[2,3-b][1,4]oxazin-7-yl}-5H,6H,7H,8H-pyrido[3,4-d]pyrimidin-2-yl) amino]phenyl}acetic acid (230.00 mg; 0.41 mmol) and HATU (331.00 mg; 0.83 mmol) in portions at 25° C. under nitrogen atmosphere. The resulting mixture was stirred for 2 h at 25° C. under nitrogen atmosphere. The resulting mixture was diluted with water, extracted with EtOAc. The combined organic layers were washed with brine and dried over anhydrous Na₂SO₄. The resulting mixture was concentrated under reduced pressure. The residue was purified by silica gel column, eluted with 20% MeOH in DCM to afford tert-butyl 7-[2-({4-[2-(3-fluoroazetidin-1-yl)-2-oxoethyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate (230.00 mg; 94.3%) as a yellow solid.

1-(3-fluoroazetidin-1-yl)-2-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}ethan-1-one: A solution of tert-butyl 7-[2-({4-[2-(3-fluoroazetidin-1-yl)-2-oxoethyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate (190.00 mg; 0.32 mmol) in TFE (6.00 ml) was stirred for 4 h at 120 degrees Celsius. The crude mixture was purified with reverse phase. Column: XBridge Shield RP18 OBD Column, 30*150 mm, 5 μm; Mobile Phase A: 10 mmol NH₄HCO₃+0.05% NH₄OH, Mobile Phase B: ACN; Flow rate: 60 mL/min; Gradient: 20% B to 50% B in 9 min, Wavelength: 254 nm; RT1 (min): 7. This resulted in 1-(3-fluoroazetidin-1-yl)-2-{4-[(7-{8-methyl-1H,2H,3H-pyrido[2,3-b][1,4]oxazin-7-yl}-5H,6H,7H,8H-pyrido[3,4-d]pyrimidin-2-yl) amino]phenyl}ethan-1-one (54.80 mg; 33.7%) as an orange solid.

HPLC: 97.03% purity, RT=3.91 min. MS: m/z=490.2 [M+H]⁺. ¹H NMR (300 MHz, DMSO-d6) 8.32 (s, 1H), 7.67 (dd, J=8.5, 1.7 Hz, 2H), 7.28 (s, 1H), 7.15-7.06 (m, 2H), 5.38 (ddt, J=57.2, 6.1, 3.0 Hz, 1H), 4.55-4.40 (m, 1H), 4.30-4.08 (m, 4H), 3.99-3.80 (m, 3H), 3.37 (s, 2H), 3.29 (s, 2H), 3.12 (s, 2H), 2.79 (s, 2H), 2.04 (s, 3H).

### Example 156: Synthesis of Compound 3-methyl-6-[(7-{8-methyl-1H,2H,3H-pyrido[2,3-b][1,4]oxazin-7-yl}-5H,6H,7H,8H-pyrido[3,4-d]pyrimidin-2-yl)amino]-3,4-dihydro-1H-2?6,3-benzothiazine-2,2-dione

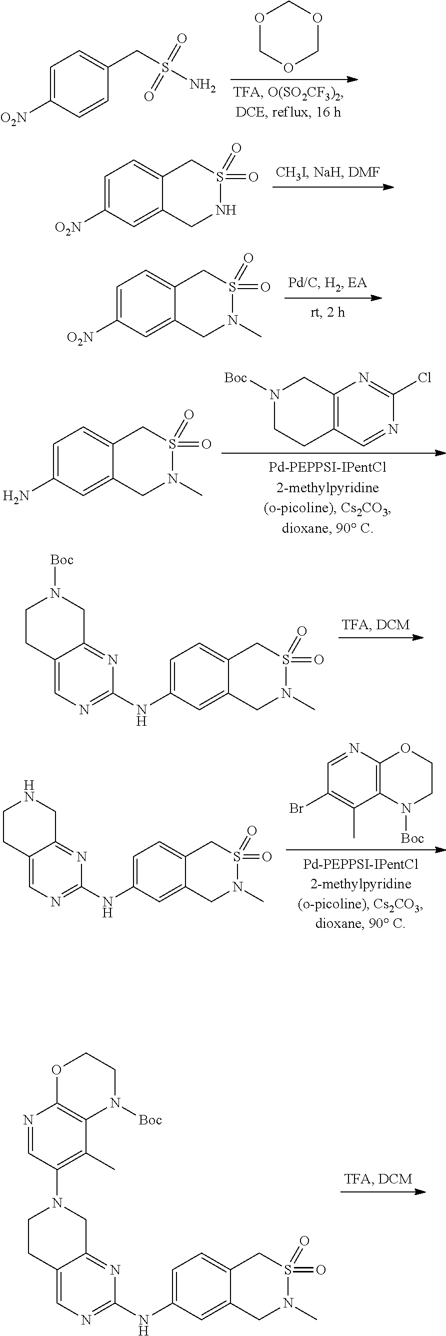

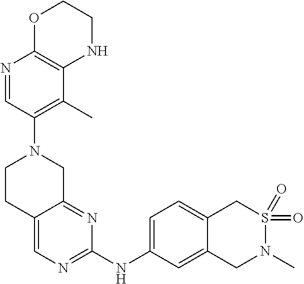 [SMILES: Cc1c(N2CCc3cnc(Nc4ccc5c(c4)CN(C)S(=O)(=O)C5)nc3C2)cnc2c1NCCO2]

6-nitro-3,4-dihydro-1H-2-6,3-benzothiazine-2,2-dione: To a solution of (4-nitrophenyl) methanesulfonamide (4.00 g; 17.58 mmol) in 1,1-dichloroethane (50.00 ml) were added CF₃SO₃H (7.00 ml; 75.15 mmol), Tf₂O (7.00 ml; 39.36 mmol) and (HCHO)n (556.00 mg; 17.59 mmol). The resulting mixture was stirred for 2 h at 35° C. and refluxed overnight under nitrogen atmosphere. The mixture was basified to pH [7] with saturated NaHCO₃ (aq.). The resulting mixture was extracted with DCM. The combined organic layers were washed with NaCl(aq.), dried over anhydrous Na₂SO₄. After filtration, the resulting mixture was concentrated under reduced pressure. The residue was purified by silica gel column chromatography, eluted with DCM/MeOH (15:1) to afford 6-nitro-3,4-dihydro-1H-2?6,3-benzothiazine-2,2-dione (730.00 mg; 14.9%) as a gray solid.

3-methyl-6-nitro-3,4-dihydro-1H-2-6,3-benzothiazine-2,2-dione: To a stirred solution of 6-nitro-3,4-dihydro-1H-2-6,3-benzothiazine-2,2-dione (700.00 mg; 2.52 mmol) in DMF (10.00 ml) was added NaH (152.00 mg; 3.80 mmol) at 0° C. under nitrogen atmosphere. The resulting mixture was stirred for 10 min at 0° C. under nitrogen atmosphere. After 10 min CH₃I (452.00 mg; 3.03 mmol) was added to the above solution at 0° C. under nitrogen atmosphere. Then the resulting mixture was stirred for additional 2 h at room temperature. The reaction was quenched with H₂O at room temperature, The resulting mixture was extracted with EtOAc. The combined organic layers were washed with NaCl(aq.), dried over anhydrous Na₂SO₄. After filtration, the filtrate was concentrated under reduced pressure to afford 3-methyl-6-nitro-3,4-dihydro-1H-2?6,3-benzothiazine-2,2-dione (580.00 mg; crude product) as a gray solid.

6-amino-3-methyl-3,4-dihydro-1H-2-6,3-benzothiazine-2,2-dione: A solution of 3-methyl-6-nitro-3,4-dihydro-1H-2-6,3-benzothiazine-2,2-dione (550.00 mg; 1.47 mmol) in ethyl acetate (10.00 ml) was added Pd/C (10.00 mg; 0.01 mmol). The resulting mixture was stirred for 2 h at 25° C. under hydrogen atmosphere. The resulting mixture was filtered, the filter cake was washed with 300 mL of EA. The filtrate was concentrated under reduced pressure to afford 6-amino-3-methyl-3,4-dihydro-1H-2?6,3-benzothiazine-2,2-dione (360.00 mg; crude product) as a yellow solid.

tert-butyl 2-[(3-methyl-2,2-dioxo-3,4-dihydro-1H-2-6,3-benzothiazin-6-yl) amino]-5H,6H,7H,8H-pyrido[3,4-d]pyrimidine-7-carboxylate: To a solution of tert-butyl 2-chloro-5H,6H,7H,8H-pyrido[3,4-d]pyrimidine-7-carboxylate (304.00 mg; 1.13 mmol) and 6-amino-3-methyl-3,4-dihydro-1H-2-6,3-benzothiazine-2,2-dione (350.00 mg; 1.12 mmol) in Dioxane-1,4 (10.00 ml) were added Cs₂CO₃ (771.00 mg; 2.25 mmol) and Pd-PEPPSI-IPentCl 2-methylpyridine (o-picoline) (100.00 mg; 0.11 mmol). The resulting mixture was stirred for overnight at 90° C. under nitrogen atmosphere. The resulting mixture was concentrated under reduced pressure. The residue was purified by silica gel column chromatography, eluted with PE/EA (1:2) to afford tert-butyl 2-[(3-methyl-2,2-dioxo-3,4-dihydro-1H-2-6,3-benzothiazin-6-yl)amino]-5H,6H,7H,8H-pyrido[3,4-d]pyrimidine-7-carboxylate (560.00 mg; 87.5%) as a white solid.

3-methyl-6-({5H,6H,7H,8H-pyrido[3,4-d]pyrimidin-2-yl}amino)-3,4-dihydro-1H-2-6,3-benzothiazine-2,2-dione: To a stirred solution of tert-butyl 2-[(3-methyl-2,2-dioxo-3,4-dihydro-1H-2-6,3-benzothiazin-6-yl)amino]-5H,6H,7H,8H-pyrido[3,4-d]pyrimidine-7-carboxylate (550.00 mg; 0.97 mmol) in DCM (10.00 ml) was added TFA (2.00 ml) in portions at room temperature. The mixture was basified to pH [7] with saturated NaHCO₃ (aq.). The resulting mixture was extracted with DCM. The combined organic layers were washed with NaCl(aq.), dried over anhydrous Na₂SO₄. After filtration, the filtrate was concentrated under reduced pressure to afford 3-methyl-6-({5H,6H,7H,8H-pyrido[3,4-d]pyrimidin-2-yl}amino)-3,4-dihydro-1H-2-6,3-benzothiazine-2,2-dione (440.00 mg; crude product) as a yellow solid.

tert-butyl 8-methyl-7-{2-[(3-methyl-2,2-dioxo-3,4-dihydro-1H-2-6,3-benzothiazin-6-yl) amino]-5H,6H,7H,8H-pyrido[3,4-d]pyrimidin-7-yl}-1H,2H,3H-pyrido[2,3-b][1,4]oxazine-1-carboxylate: To a solution of 3-methyl-6-({5H,6H,7H,8H-pyrido[3,4-d]pyrimidin-2-yl}amino)-3,4-dihydro-1H-2-6,3-benzothiazine-2,2-dione (430.00 mg; 0.84 mmol) and tert-butyl 7-bromo-8-methyl-1H,2H,3H-pyrido[2,3-b][1,4]oxazine-1-carboxylate (298.00 mg; 0.85 mmol) in Dioxane-1,4 (10.00 ml) were added Cs₂CO₃ (580.00 mg; 1.69 mmol) and Pd-PEPPSI-IPentCl 2-methylpyridine (o-picoline) (75.00 mg; 0.08 mmol). The resulting mixture was stirred for overnight at 90° C. under nitrogen atmosphere. The resulting mixture was concentrated under vacuum. The residue was purified by silica gel column chromatography, eluted with PE/EtOAc (1:3) to afford tert-butyl 8-methyl-7-{2-[(3-methyl-2,2-dioxo-3,4-dihydro-1H-2?6,3-benzothiazin-6-yl) amino]-5H,6H,7H,8H-pyrido[3,4-d]pyrimidin-7-yl}-1H,2H,3H-pyrido[2,3-b][1,4]oxazine-1-carboxylate (400.00 mg; 53.4%) as a yellow solid.

3-methyl-6-[(7-{8-methyl-1H,2H,3H-pyrido[2,3-b][1,4]oxazin-7-yl}-5H,6H,7H,8H-pyrido[3,4-d]pyrimidin-2-yl) amino]-3,4-dihydro-1H-2-6,3-benzothiazine-2,2-dione: To a stirred solution of tert-butyl 8-methyl-7-{2-[(3-methyl-2,2-dioxo-3,4-dihydro-1H-2-6,3-benzothiazin-6-yl) amino]-5H,6H,7H,8H-pyrido[3,4-d]pyrimidin-7-yl}-1H,2H,3H-pyrido[2,3-b][1,4]oxazine-1-carboxylate (380.00 mg; 0.43 mmol) in DCM (8.00 ml) was added TFA (2.00 ml) in portions at room temperature. The resulting mixture was stirred for 6 h at 25° C. under nitrogen atmosphere. The mixture was basified to pH [7] with saturated NaHCO₃ (aq.). The resulting mixture was extracted with DCM. The combined organic layers were washed with NaCl(aq.), dried over anhydrous Na₂SO₄. After filtration, the filtrate was concentrated under reduced pressure. The residue was purified by Prep-HPLC with following conditions (Column: Xselect CSH C18 OBD Column 30*150 mm 5 μm; Mobile Phase A: Water (0.1% FA), Mobile Phase B: ACN; Flow rate: 60 mL/min; Gradient: 25% B to 55% B in 8 min, Wavelength: 254 nm; RT1 (min): 7) to afford 3-methyl-6-[(7-{8-methyl-1H,2H,3H-pyrido[2,3-b][1,4]oxazin-7-yl}-5H,6H,7H,8H-pyrido[3,4-d]pyrimidin-2-yl)amino]-3,4-dihydro-1H-2-6,3-benzothiazine-2,2-dione (15.60 mg; 6.8%) as a white solid.

HPLC: 91.73% purity, RT=6.38 min. MS: m/z=494.0 [M+H]⁺. ¹H NMR (300 MHz, DMSO-d6) δ 9.64 (s, 1H), 8.35 (s, 1H), 7.65 (d, J=13.1 Hz, 2H), 7.29 (s, 1H), 7.03 (d, J=8.4 Hz, 1H), 5.53 (s, 1H), 4.45 (d, J=17.4 Hz, 4H), 4.19 (s, 2H), 3.97 (s, 2H), 3.33 (s, 2H), 3.13 (s, 2H), 2.78 (d, J=9.5 Hz, 5H), 2.04 (s, 3H).

### Example 157 & 158: Synthesis of Compound N-{4-[(1S)-1-methanesulfonylethyl]phenyl}-7-{8-methyl-1H,2H,3H-pyrido[2,3-b][1,4]oxazin-7-yl}-5H,6H,7H,8H-pyrido[3,4-d]pyrimidin-2-amine and N-{4-[(1R)-1-methanesulfonylethyl]phenyl}-7-{8-methyl-1H,2H,3H-pyrido[2,3-b][1,4]oxazin-7-yl}-5H,6H,7H,8H-pyrido[3,4-d]pyrimidin-2-amine

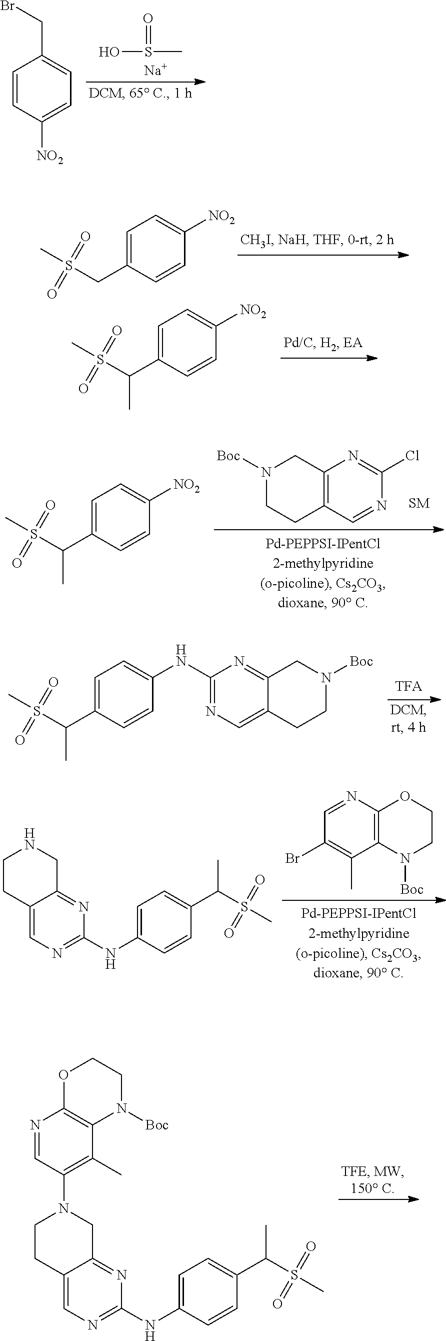

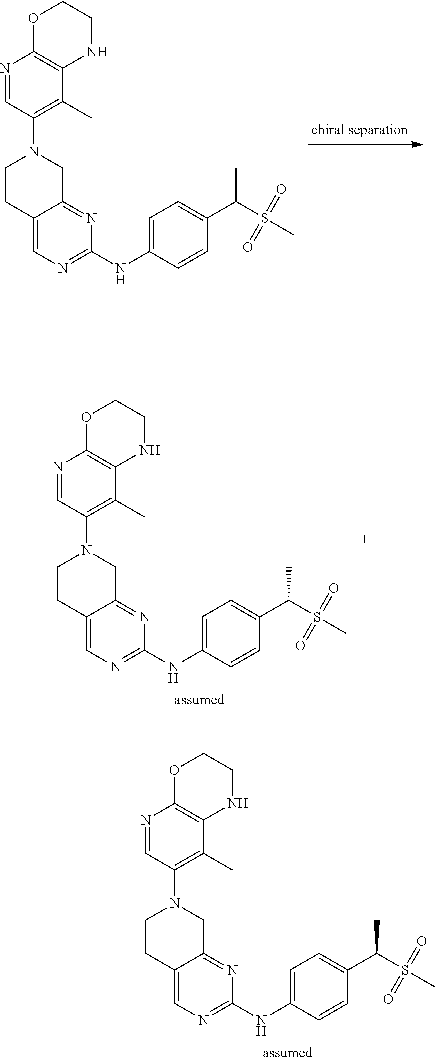 [SMILES: Cc1c(N2CCc3cnc(Nc4ccc(C(C)S(C)(=O)=O)cc4)nc3C2)cnc2c1NCCO2 ; Cc1c(N2CCc3cnc(Nc4ccc([C@H](C)S(C)(=O)=O)cc4)nc3C2)cnc2c1NCCO2 ; Cc1c(N2CCc3cnc(Nc4ccc([C@@H](C)S(C)(=O)=O)cc4)nc3C2)cnc2c1NCCO2]

1-(methanesulfonylmethyl)-4-nitrobenzene: To a stirred solution of 1-(bromomethyl)-4-nitrobenzene (10.00 g; 43.97 mmol;) in DMF (100.00 ml) was added sodium methanesulfonic acid (7.94 g; 66.00 mmol). Then the resulting mixture was stirred for additional 2 h at 65° C. The reaction was quenched with H₂O. The resulting mixture was filtered, the filtered cake was washed with H₂O (2×30 mL). The filtrate was concentrated under reduced pressure to afford 1-(methanesulfonylmethyl)-4-nitrobenzene (8.80 g; crude product) as a white solid.

1-(1-methanesulfonylethyl)-4-nitrobenzene: To a stirred solution of 1-(methanesulfonylmethyl)-4-nitrobenzene (1000.00 mg; 4.18 mmol) in DMF (20.00 ml) was added NaH (251.00 mg; 6.28 mmol) at 0° C. under nitrogen atmosphere. The resulting mixture was stirred for 10 min at 0° C. under nitrogen atmosphere. After 10 min, CH₃I (0.33 ml; 5.02 mmol) was added to the above solution at 0° C. under nitrogen atmosphere. Then the resulting mixture was stirred for additional 2 h at room temperature. The reaction was quenched by the addition of H₂O at room temperature and extracted by 3×30 ml of EA. The combined organic layers were washed with 3×30 ml brine, dried over anhydrous Na₂SO₄. After filtration, the filtrate was concentrated under reduced pressure. The residue was purified by silica gel column chromatography, eluted with PE/EA (5:2) to afford 1-(1-methanesulfonylethyl)-4-nitrobenzene (520.00 mg; 50.9%) as a yellow solid.

4-(1-methanesulfonylethyl)aniline: A solution of 1-(1-methanesulfonylethyl)-4-nitrobenzene (500.00 mg; 2.05 mmol) in ethyl acetate (10.00 ml) was added Pd/C (10.00 mg; 0.01 mmol). The resulting mixture was stirred for 2 h at 25° C. under hydrogen atmosphere. The resulting mixture was filtered, the filter cake was washed with 300 mL of EA. The filtrate was concentrated under reduced pressure to afford 4-(1-methanesulfonylethyl)aniline (410.00 mg; 80.4%) as a yellow solid.

tert-butyl 2-{[4-(1-methanesulfonylethyl)phenyl]amino}-5H,6H,7H,8H-pyrido[3,4-d]pyrimidine-7-carboxylate: To a solution of tert-butyl 2-chloro-5H,6H,7H,8H-pyrido[3,4-d]pyrimidine-7-carboxylate (412.00 mg; 1.53 mmol) and 4-(1-methanesulfonylethyl) aniline (380.00 mg; 1.52 mmol) in Dioxane-1,4 (10.00 ml) were added Cs₂CO₃ (1046.00 mg; 3.05 mmol) and Pd-PEPPSI-IPentCl 2-methylpyridine (o-picoline) (135.00 mg; 0.15 mmol). The resulting mixture was stirred for overnight at 90° C. under nitrogen atmosphere. The resulting mixture was concentrated under reduced pressure. The residue was purified by silica gel column chromatography, eluted with DCM/MeOH (19:1) to afford tert-butyl 2-{[4-(1-methanesulfonylethyl)phenyl]amino}-5H,6H,7H,8H-pyrido[3,4-d]pyrimidine-7-carboxylate (760.00 mg; 99.6%) as a yellow solid.

N-[4-(1-methanesulfonylethyl)phenyl]-5H,6H,7H,8H-pyrido[3,4-d]pyrimidin-2-amine: To a stirred solution of tert-butyl 2-{[4-(1-methanesulfonylethyl)phenyl]amino}-5H,6H,7H,8H-pyrido[3,4-d]pyrimidine-7-carboxylate (750.00 mg; 1.50 mmol) in DCM (10.00 ml) was added TFA (2.00 ml) in portions at room temperature. The mixture was basified to pH [7] with saturated NaHCO₃ (aq.). The resulting mixture was extracted with DCM. The combined organic layers were washed with NaCl(aq.), dried over anhydrous Na₂SO₄. After filtration, the filtrate was concentrated under reduced pressure to afford N-[4-(1-methanesulfonylethyl)phenyl]-5H,6H,7H,8H-pyrido[3,4-d]pyrimidin-2-amine (600.00 mg; 90.6%) as a yellow solid.

tert-butyl 7-(2-{[4-(1-methanesulfonylethyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate: To a solution of N-[4-(1-methanesulfonylethyl) phenyl]-5H,6H,7H,8H-pyrido[3,4-d]pyrimidin-2-amine (580.00 mg; 1.31 mmol) and tert-butyl 7-bromo-8-methyl-1H,2H,3H-pyrido[2,3-b][1,4]oxazine-1-carboxylate (462.00 mg; 1.31 mmol) in Dioxane-1,4 (10.00 ml) were added Cs₂CO₃ (900.00 mg; 2.62 mmol) and Pd-PEPPSI-IPentCl 2-methylpyridine (o-picoline) (117.00 mg; 0.13 mmol). The resulting mixture was stirred for overnight at 90° C. under nitrogen atmosphere. The resulting mixture was concentrated under reduced pressure. The residue was purified by silica gel column chromatography, eluted with DCM/MeOH (19:1) to afford tert-butyl 7-(2-{[4-(1-methanesulfonylethyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (580.00 mg; 71.3%) as a yellow solid.

N-[4-(1-methanesulfonylethyl)phenyl]-7-{8-methyl-1H,2H,3H-pyrido[2,3-b][1,4]oxazin-7-yl}-5H,6H,7H,8H-pyrido[3,4-d]pyrimidin-2-amine: A solution of tert-butyl 7-(2-{[4-(1-methanesulfonylethyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (550.00 mg; 0.89 mmol) in TFE (5.00 ml) was microwave radiation for 2 h at 150° C. The mixture was concentrated under reduced pressure. The residue was purified by Prep-HPLC with following conditions (Column: XBridge Prep OBD C18 Column, 30*150 mm, 5 μm; Mobile Phase A: Water (10 mmol/L NH₄HCO₃+0.05% NH₄OH), Mobile Phase B: ACN; Flow rate: 60 mL/min; Gradient: 30% B to 60% B in 8 min, Wavelength: 254 nm; RT1 (min): 7) to afford N-[4-(1-methanesulfonylethyl)phenyl]-7-{8-methyl-1H,2H,3H-pyrido[2,3-b][1,4]oxazin-7-yl}-5H,6H,7H,8H-pyrido[3,4-d]pyrimidin-2-amine (210.00 mg; 49.2%) as a yellow solid.

N-{4-[(1S)-1-methanesulfonylethyl]phenyl}-7-{8-methyl-1H,2H,3H-pyrido[2,3-b][1,4]oxazin-7-yl}-5H,6H,7H,8H-pyrido[3,4-d]pyrimidin-2-amine and N-{4-[(1R)-1-methanesulfonylethyl]phenyl}-7-{8-methyl-1H,2H,3H-pyrido[2,3-b][1,4]oxazin-7-yl}-5H,6H,7H,8H-pyrido[3,4-d]pyrimidin-2-amine: The N-[4-(1-methanesulfonylethyl)phenyl]-7-{8-methyl-1H,2H,3H-pyrido[2,3-b][1,4]oxazin-7-yl}-5H,6H,7H,8H-pyrido[3,4-d]pyrimidin-2-amine (210.00 mg; 0.44 mmol) was purified by Prep-Chiral-HPLC(Column: DZ-CHIRALPAK IE-3, 4.6*50 mm, 3.0 μm; Mobile Phase A: (Hex: DCM=1: 1)(0.1% DEA): IPA=50: 50; Flow rate: 1 mL/min; Injection Volume: 5 ul mL) to afford N-{4-[(1S)-1-methanesulfonylethyl]phenyl}-7-{8-methyl-1H,2H,3H-pyrido[2,3-b][1,4]oxazin-7-yl}-5H,6H,7H,8H-pyrido[3,4-d]pyrimidin-2-amine (69.90 mg; 33.1%) as an off-white solid and N-{4-[(1R)-1-methanesulfonylethyl]phenyl}-7-{8-methyl-1H,2H,3H-pyrido[2,3-b][1,4]oxazin-7-yl}-5H,6H,7H,8H-pyrido[3,4-d]pyrimidin-2-amine (67.00 mg; 31.4%) as an off-white solid.

Compound 157 (assumed stereochemistry): HPLC: 99.27% purity, RT=2.88 min. MS: m/z=481.0 [M+H]⁺. ¹H NMR (300 MHz, DMSO-d6) δ 9.64 (s, 1H), 8.35 (s, 1H), 7.78 (d, J=8.6 Hz, 2H), 7.47-7.17 (m, 3H), 5.53 (s, 1H), 4.43 (q, J=7.1 Hz, 1H), 4.29-4.05 (m, 2H), 3.98 (s, 2H), 3.31 (s, 2H), 3.13 (t, J=5.6 Hz, 2H), 2.79 (d, J=5.9 Hz, 5H), 2.51 (p, J=1.8 Hz, 3H), 2.05 (s, 3H).

Compound 158 (assumed stereochemistry): HPLC: 98.36% purity, RT=2.86 min. MS: m/z=481.0 [M+H]⁺. ¹H NMR (300 MHz, DMSO-d6) δ 9.64 (s, 1H), 8.35 (s, 1H), 7.78 (d, J=8.7 Hz, 2H), 7.33 (d, J=8.6 Hz, 2H), 7.30 (s, 1H), 5.53 (s, 1H), 4.43 (q, J=7.1 Hz, 1H), 4.20 (t, J=4.4 Hz, 2H), 3.98 (s, 2H), 3.31 (s, 2H), 3.13 (t, J=5.7 Hz, 2H), 2.79 (d, J=6.4 Hz, 5H), 2.05 (s, 3H), 1.61 (d, J=7.1 Hz, 3H).

### Example 159: Synthesis of Compound 7-{8-chloro-1H,2H,3H-pyrido[2,3-b][1,4]oxazin-7-yl}-N-[4-(5-cyclopropyl-1,3,4-oxadiazol-2-yl) henyl]-5H,6H,7H,8H-pyrido[3,4-d]pyrimidin-2-amine

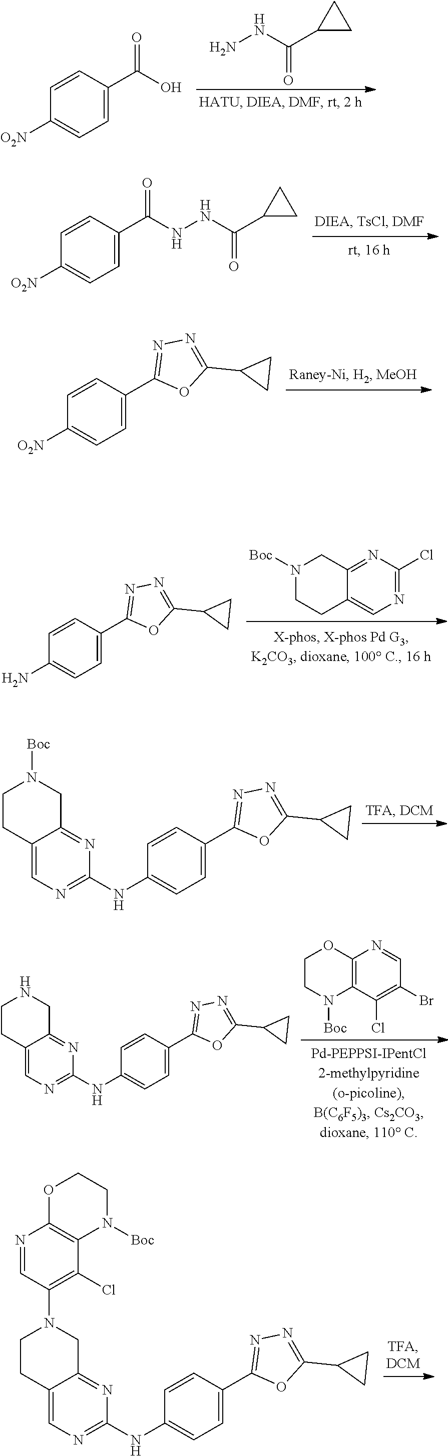

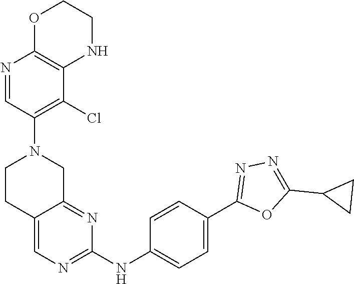 [SMILES: Clc1c(N2CCc3cnc(Nc4ccc(-c5nnc(C6CC6)o5)cc4)nc3C2)cnc2c1NCCO2]

N′-cyclopropanecarbonyl-4-nitrobenzohydrazide: To a stirred mixture of 4-nitrobenzoic acid (5.00 g; 24.35 mmol) and cyclopropanecarbohydrazide (3.04 g; 24.35 mmol) in DMF (50.00 ml) were added HATU (29.24 g; 73.06 mmol) and DIEA (6.62 g; 48.71 mmol) at room temperature. The mixture was stirred for 1 h at room temperature. The resulting mixture was filtered, the filtered cake was washed with water. After filtration, the cake was concentrated under reduced pressure. This resulted in N′-yclopropanecarbonyl-4-nitrobenzohydrazide (6.00 g; crude product) as a yellow solid.

2-cyclopropyl-5-(4-nitrophenyl)-1,3,4-oxadiazole: To a stirred mixture of N′-cyclopropanecarbonyl-4-nitrobenzohydrazide (6.00 g; 19.60 mmol) and DIEA (8.40 ml) in DMF (100.00 ml) was added TsCl (5.00 g; 24.91 mmol) at room temperature. The mixture was stirred for 2 h at room temperature. The resulting mixture was quenched by ice/water and extracted with EtOAc. The combined organic layers were washed with brine, dried over anhydrous Na₂SO₄. After filtration, the filtrate was concentrated under reduced pressure. This resulted in 2-cyclopropyl-5-(4-nitrophenyl)-1,3,4-oxadiazole (1.00 g; 21.4%) as a green solid.

4-(5-cyclopropyl-1,3,4-oxadiazol-2-yl) aniline: To a solution of 2-cyclopropyl-5-(4-nitrophenyl)-1,3,4-oxadiazole (1.00 g; 4.25 mmol) in MeOH (30.00 mL) was added Raney-Ni (30.00 mg; 0.33 mmol) at room temperature. After stirring for 2 h at room temperature under hydrogen atmosphere. The resulting mixture was filtered, the filter cake was washed with MeOH. The crude product was used in the next step directly without further purification. This resulted in 4-(5-cyclopropyl-1,3,4-oxadiazol-2-yl) aniline (800.00 mg; 92.5%) as a yellow solid.

tert-butyl 2-{[4-(5-cyclopropyl-1,3,4-oxadiazol-2-yl)phenyl]amino}-5H,6H,7H,8H-pyrido[3,4-d]pyrimidine-7-carboxylate: To a stirred mixture of tert-butyl 2-chloro-5H,6H,7H,8H-pyrido[3,4-d]pyrimidine-7-carboxylate (400.00 mg; 1.44 mmol) and 4-(5-cyclopropyl-1,3,4-oxadiazol-2-yl) aniline (359.65 mg; 1.73 mmol) in 1,4-dioxane (10.00 ml) were added X-PHOS (145.00 mg; 0.29 mmol), K₂CO₃ (420.34 mg; 2.89 mmol) and XPhos Pd G₃ (128.55 mg; 0.14 mmol) in portions at 25° C. The resulting mixture was stirred for 2 h at 100° C. under nitrogen atmosphere. The resulting mixture was concentrated under vacuum. The residue was purified by silica gel column chromatography, eluted with DCM:MeOH=10:1 to afford tert-butyl 2-{[4-(5-cyclopropyl-1,3,4-oxadiazol-2-yl)phenyl]amino}-5H,6H,7H,8H-pyrido[3,4-d]pyrimidine-7-carboxylate (510.00 mg; 72.0%) as a yellow solid.

N-[4-(5-cyclopropyl-1,3,4-oxadiazol-2-yl)phenyl]-5H,6H,7H,8H-pyrido[3,4-d]pyrimidin-2-amine: To a stirred mixture of tert-butyl 2-{[4-(5-cyclopropyl-1,3,4-oxadiazol-2-yl)phenyl]amino}-5H,6H,7H,8H-pyrido[3,4-d]pyrimidine-7-carboxylate (510.00 mg; 1.17 mmol) in DCM (10.00 ml) was added TFA (2.00 ml; 26.93 mmol) in portions at 25° C. The resulting mixture was stirred for 2 h at 25° C. under nitrogen atmosphere. The mixture was basified to pH=9 with sat. NaHCO₃(aq.) and extracted with EtOAc. The combined organic layers were washed with brine, dried over anhydrous Na₂SO₄. After filtration, the filtrate was concentrated under reduced pressure. This resulted in N-[4-(5-cyclopropyl-1,3,4-oxadiazol-2-yl) phenyl]-5H,6H,7H,8H-pyrido[3,4-d]pyrimidin-2-amine (340.00 mg; 85.3%) as a yellow oil.

tert-butyl 8-chloro-7-(2-{[4-(5-cyclopropyl-1,3,4-oxadiazol-2-yl)phenyl]amino}-5H,6H,7H,8H-pyrido[3,4-d]pyrimidin-7-yl)-1H,2H,3H-pyrido[2,3-b][1,4]oxazine-1-carboxylate: To a stirred mixture of N-[4-(5-cyclopropyl-1,3,4-oxadiazol-2-yl)phenyl]-5H,6H,7H,8H-pyrido[3,4-d]pyrimidin-2-amine (330.00 mg; 0.80 mmol) and tert-butyl 7-bromo-8-chloro-1H,2H,3H-pyrido[2,3-b][1,4]oxazine-1-carboxylate (337.03 mg; 0.96 mmol) in 1,4-dioxane (12.00 ml) were added Pd-PEPPSI-IPentCl 2-methylpyridine (o-picoline) (71.05 mg; 0.08 mmol) and Cs₂CO₃ (551.05 mg; 1.61 mmol) and tris(2,3,4,5,6-pentafluorophenyl)borane (83.94 mg; 0.16 mmol.) at room temperature. The mixture was stirred for 16 h at 110° C. The resulting mixture was concentrated under reduced pressure and purified by silica gel column chromatography, eluted with CH₂Cl₂/MeOH (1:1) to afford tert-butyl 8-chloro-7-(2-{[4-(5-cyclopropyl-1,3,4-oxadiazol-2-yl)phenyl]amino}-5H,6H,7H,8H-pyrido[3,4-d]pyrimidin-7-yl)-1H,2H,3H-pyrido[2,3-b][1,4]oxazine-1-carboxylate (270.00 mg; 40.8%) as a yellow solid.

7-{8-chloro-1H,2H,3H-pyrido[2,3-b][1,4]oxazin-7-yl}-N-[4-(5-cyclopropyl-1,3,4-oxadiazol-2-yl)phenyl]-5H,6H,7H,8H-pyrido[3,4-d]pyrimidin-2-amine: To a stirred mixture of tert-butyl 8-chloro-7-(2-{[4-(5-cyclopropyl-1,3,4-oxadiazol-2-yl)phenyl]amino}-5H,6H,7H,8H-pyrido[3,4-d]pyrimidin-7-yl)-1H,2H,3H-pyrido[2,3-b][1,4]oxazine-1-carboxylate (260.00 mg; 0.38 mmol) and TFA (2.00 ml) in DCM (10.00 ml) at room temperature. The mixture was stirred for 1 h at room temperature. The resulting mixture was concentrated under reduced pressure. The residue was purified by Prep-HPLC(Column: XBridge Shield RP18 OBD Column, 30*150 mm, 5 μm; Mobile Phase A: Water (10 mmol/L NH₄HCO₃+0.05% NH₄OH), Mobile Phase B: ACN; Flow rate: 60 mL/min; Gradient: 33% to 63% in 9 min; Wavelength: 254 nm; RT1 (min): 7) to afford 7-{8-chloro-1H,2H,3H-pyrido[2,3-b][1,4]oxazin-7-yl}-N-[4-(5-cyclopropyl-1,3,4-oxadiazol-2-yl)phenyl]-5H,6H,7H,8H-pyrido[3,4-d]pyrimidin-2-amine (92.50 mg; 48.4%) as a white solid.

HPLC: 99.90% purity, RT=4.24 min. MS: m/z=503.10 [M+H]⁺. ¹H NMR (300 MHz, DMSO-d6): 10.01 (s, 1H), 8.43 (s, 1H), 8.02-7.93 (m, 2H), 7.90-7.80 (m, 2H), 7.33 (s, 1H), 6.13 (s, 1H), 4.24 (d, J=4.8 Hz, 2H), 4.13 (s, 2H), 3.33-3.23 (m, 2H), 3.22 (m, 2H), 2.85 (s, 2H), 2.28 (tt, J=8.3, 5.1 Hz, 1H), 1.27-1.10 (m, 4H).

### Example 160: Synthesis of Compound 6-(2-{[4-(methanesulfonylmethyl)phenyl]amino}-5H,6H,7H,8H-pyrido[3,4-d]pyrimidin-7-yl)-7-methyl-1H,2H-[1,3]oxazolo[5,4-b]pyridin-2-one

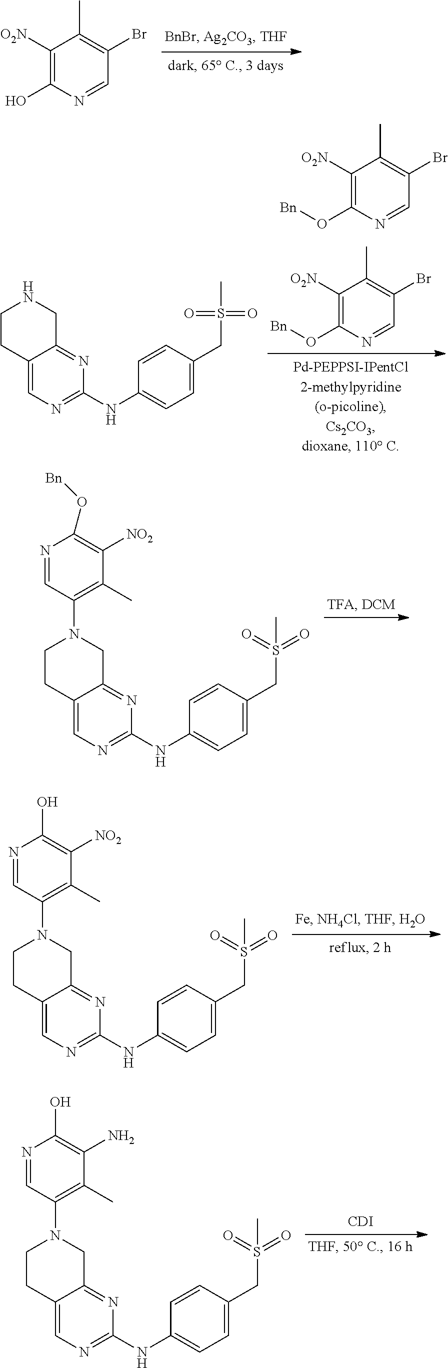

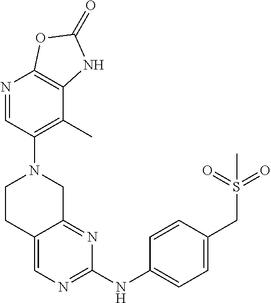 [SMILES: Cc1c(N2CCc3cnc(Nc4ccc(CS(C)(=O)=O)cc4)nc3C2)cnc2oc(=O)[nH]c12]

2-(benzyloxy)-5-bromo-4-methyl-3-nitropyridine: To a stirred solution of 5-bromo-4-methyl-3-nitropyridin-2-ol (2.50 g; 10.19 mmol) in THF (30.00 ml) were added BnBr (2.20 g; 12.23 mmol) and Ag₂CO₃ (3.55 g; 12.23 mmol) in portions at room temperature under N₂ atmosphere. The resulting mixture was stirred for 2 days at 65° C. in dark room. The residue was purified by silica gel column, eluted with 80% EtOAc in PE to afford crude product. The crude product was repurified by C18 flash column eluted with 97% ACN in water to afford 2-(benzyloxy)-5-bromo-4-methyl-3-nitropyridine (1.70 g; 46.8%) as a yellow solid.

7-[6-(benzyloxy)-4-methyl-5-nitropyridin-3-yl]-N-[4-(methanesulfonylmethyl)phenyl]-5H,6H,7H,8H-pyrido[3,4-d]pyrimidin-2-amine: To a stirred solution of 2-(benzyloxy)-5-bromo-4-methyl-3-nitropyridine (1.10 g; 3.21 mmol) and N-[4-(methanesulfonylmethyl)phenyl]-5H,6H,7H,8H-pyrido[3,4-d]pyrimidin-2-amine (1.00 g; 2.99 mmol) in Dioxane-1,4 (15.00 ml) was added Pd-PEPPSI-IPentCl 2-methylpyridine (o-picoline) (265.00 mg; 0.30 mmol.) and Cs₂CO₃ (2.05 g; 5.98 mmol) at room temperature. The resulting mixture was stirred for 3 h at 90° C. under nitrogen atmosphere. The resulting mixture was concentrated under reduced pressure. The residue was purified by silica gel column chromatography, eluted with PE/EA (1:6) to afford 7-[6-(benzyloxy)-4-methyl-5-nitropyridin-3-yl]-N-[4-(methanesulfonylmethyl)phenyl]-5H,6H,7H,8H-pyrido[3,4-d]pyrimidin-2-amine (650.00 mg; 32.6%) as a yellow solid.

5-(2-{[4-(methanesulfonylmethyl)phenyl]amino}-5H,6H,7H,8H-pyrido[3,4-d]pyrimidin-7-yl)-4-methyl-3-nitropyridin-2-ol: To a stirred solution of 7-[6-(benzyloxy)-4-methyl-5-nitropyridin-3-yl]-N-[4-(methanesulfonylmethyl) phenyl]-5H,6H,7H,8H-pyrido[3,4-d]pyrimidin-2-amine (650.00 mg; 0.97 mmol) in DCM (4.00 ml) was added TFA (1.00 ml) at room temperature. The mixture was basified to pH [7] with saturated NaHCO₃ (aq.) The resulting mixture was extracted with EtOAc. The combined organic layers were dried over anhydrous Na₂SO₄. After filtration, the filtrate was concentrated under reduced pressure to afford 5-(2-{[4-(methanesulfonylmethyl)phenyl]amino}-5H,6H,7H,8H-pyrido[3,4-d] yrimidin-7-yl)-4-methyl-3-nitropyridin-2-ol (400.00 mg; 66.3%) as a yellow solid.

3-amino-5-(2-{[4-(methanesulfonylmethyl) Phenyl]amino}-5H,6H,7H,8H-pyrido[3,4-d]pyrimidin-7-yl)-4-methylpyridin-2-ol: To a stirred solution of 5-(2-{[4-(methanesulfonylmethyl)phenyl]amino}-5H,6H,7H,8H-pyrido[3,4-d]pyrimidin-7-yl)-4-methyl-3-nitropyridin-2-ol (400.00 mg; 0.60 mmol) and NH₄Cl (137.00 mg; 2.43 mmol) in Water (4.00 ml) and EtOH (6.00 ml) were added Fe (179.00 mg; 3.04 mmol) in portions at room temperature. The resulting mixture was stirred for 2 h at 80° C. The resulting mixture was filtered, the filter cake was washed with EtOAc (100 ml). The filtrate. The resulting mixture was extracted with EtOAc (100 ml*3). The combined organic layers were washed with brine (100 ml), dried over anhydrous Na₂SO₄. After filtration, the filtrate was concentrated under reduced pressure to afford 3-amino-5-(2-{[4-(methanesulfonylmethyl) henyl] mino}-5H,6H,7H,8H-pyrido[3,4-d] yrimidin-7-yl)-4-methylpyridin-2-ol (300.00 mg; 84.1%) as a yellow solid.

6-(2-{[4-(methanesulfonylmethyl)phenyl]amino}-5H,6H,7H,8H-pyrido[3,4-d]pyrimidin-7-yl)-7-methyl-1H,2H-[1,3] oxazolo[5,4-b]pyridin-2-one: To a solution of 3-amino-5-(2-{[4-(methanesulfonylmethyl) henyl] mino}-5H,6H,7H,8H-pyrido[3,4-d] yrimidin-7-yl)-4-methylpyridin-2-ol (300.00 mg; 0.51 mmol) in THF (4.00 ml) was added CDI (131.00 mg; 0.77 mmol). The resulting mixture was stirred for 2 h at 35° C. and reflux overnight under nitrogen atmosphere. The resulting mixture was concentrated under reduced pressure. The residue was purified by Prep-HPLC with following conditions (Column: XBridge Shield RP18 OBD Column, 30*150 mm, 5 μm; Mobile Phase A: Water 10 mmol/L NH₄HCO₃+0.05 NH₄OH), Mobile Phase B: ACN; Flow rate: 60 mL/min; Gradient: 18% to 40% in 9 min; Wavelength: 254 nm; RT1 (min): 7) to afford 6-(2-{[4-(methanesulfonylmethyl)phenyl]amino}-5H,6H,7H,8H-pyrido[3,4-d]pyrimidin-7-yl)-7-methyl-1H,2H-[1,3] oxazolo[5,4-b]pyridin-2-one (25.40 mg; 10.4%) as alight yellow solid.

HPLC: 97.04% purity, RT=3.69 min. MS: m/z=467.00 [M+H]⁺. ¹H NMR (300 MHz, DMSO-d6) 11.96 (s, 1H), 9.65 (s, 1H), 8.38 (s, 1H), 7.88-7.66 (m, 3H), 7.29 (d, J=8.5 Hz, 2H), 4.38 (s, 2H), 4.09 (s, 2H), 3.23 (t, J=5.6 Hz, 2H), 2.85 (m, 5H), 2.29 (s, 3H).

### Example 161: Synthesis of Compound N-[4-(2-methanesulfonylethyl)phenyl]-7-{8-methyl-1H,2H,3H-pyrido[2,3-b][1,4]oxazin-7-yl}-5H,6H,7H,8H-pyrido[3,4-d]pyrimidin-2-amine

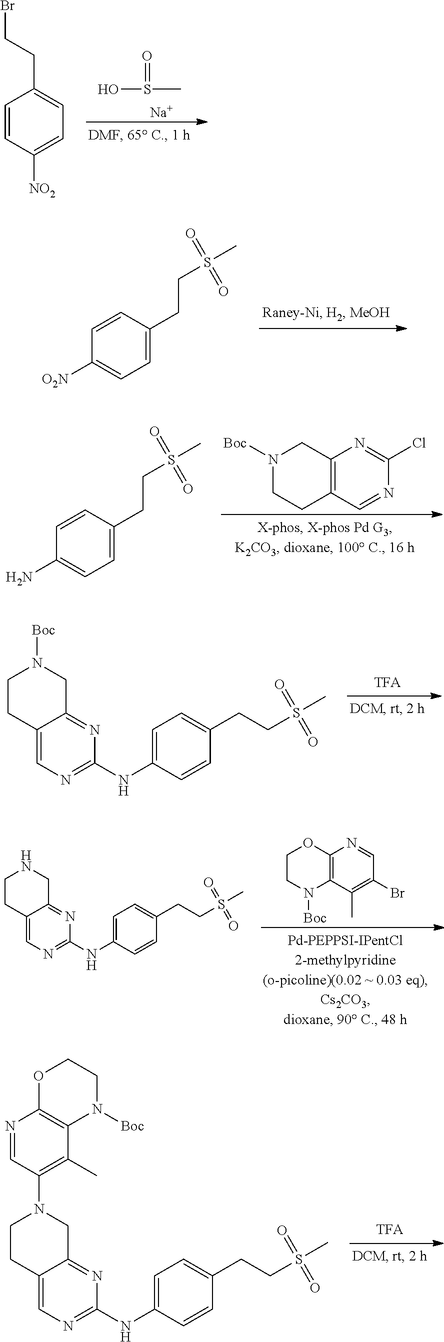

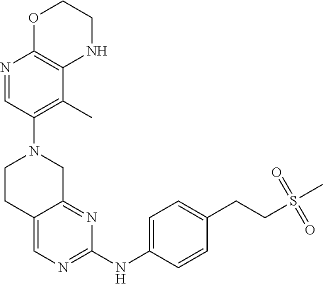 [SMILES: Cc1c(N2CCc3cnc(Nc4ccc(CCS(C)(=O)=O)cc4)nc3C2)cnc2c1NCCO2]

1-(2-methanesulfonylethyl)-4-nitrobenzene: A mixture of 1-(2-bromoethyl)-4-nitrobenzene (5.00 g; 21.17 mmol) and sodium methanesulfinate (3.49 g; 33.88 mmol) in DMF (50.00 ml) was stirred for 2 h at 60° C. The resulting mixture was quenched by ice/water and extracted with EtOAc. The combined organic layers were washed with brine, dried over anhydrous Na₂SO₄. After filtration, the filtrate was concentrated under reduced pressure. This resulted in 1-(2-methanesulfonylethyl)-4-nitrobenzene (4.00 g; 81.5%) as a white solid.

4-(2-methanesulfonylethyl) aniline: To a solution of 1-(2-methanesulfonylethyl)-4-nitrobenzene (2.00 g; 8.57 mmol) in MeOH (30.00 ml) was added Raney-Ni (30.00 mg; 0.33 mmol) at room temperature. After stirring for 2 h at room temperature under hydrogen atmosphere. The resulting mixture was filtered and concentrated under reduced pressure. The crude product was used in the next step directly without further purification. This resulted in 4-(2-methanesulfonylethyl) aniline (1.50 g; 85.3%) as a white solid.

tert-butyl 2-{[4-(2-methanesulfonylethyl)phenyl]amino}-5H,6H,7H,8H-pyrido[3,4-d]pyrimidine-7-carboxylate: To a stirred mixture of tert-butyl 2-chloro-5H,6H,7H,8H-pyrido[3,4-d]pyrimidine-7-carboxylate (400.00 mg; 1.44 mmol) and 4-(2-methanesulfonylethyl) aniline (356.15 mg; 1.73 mmol) in 1,4-dioxane (15.00 ml) were added X-PHOS (145.00 mg; 0.29 mmol), K₂CO₃ (420.34 mg; 2.89 mmol) and XPhos Pd G₃ (128.55 mg; 0.14 mmol) in portions at 25° C. The resulting mixture was stirred for 2 h at 100° C. under nitrogen atmosphere. The resulting mixture was concentrated under vacuum. The residue was purified by silica gel column chromatography, eluted with DCM:MeOH=10:1 to afford tert-butyl 2-{[4-(2-methanesulfonylethyl)phenyl]amino}-5H,6H,7H,8H-pyrido[3,4-d]pyrimidine-7-carboxylate (600.00 mg; 94.1%) as a yellow solid.

N-[4-(2-methanesulfonylethyl)phenyl]-5H,6H,7H,8H-pyrido[3,4-d]pyrimidin-2-amine: To a stirred mixture of tert-butyl 2-{[4-(2-methanesulfonylethyl) phenyl]amino}-5H,6H,7H,8H-pyrido[3,4-d]pyrimidine-7-carboxylate (510.00 mg; 1.18 mmol) in DCM (10.00 ml) was added TFA (2.00 ml) in portions at 25° C. The resulting mixture was stirred for 2 h at 25° C. under nitrogen atmosphere. The mixture was basified to pH=9 with sat. NaHCO₃(aq.) and extracted with EtOAc. The combined organic layers were washed with brine, dried over anhydrous Na₂SO₄. After filtration, the filtrate was concentrated under reduced pressure. This resulted in N-[4-(2-methanesulfonylethyl)phenyl]-5H,6H,7H,8H-pyrido[3,4-d]pyrimidin-2-amine (310.00 mg; Crude Product) as a yellow solid.

tert-butyl 7-(2-{[4-(2-methanesulfonylethyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate: To a stirred mixture of N-[4-(2-methanesulfonylethyl) phenyl]-5H,6H,7H,8H-pyrido[3,4-d]pyrimidin-2-amine (300.00 mg; 0.73 mmol) and tert-butyl 7-bromo-8-methyl-1H,2H,3H-pyrido[2,3-b][1,4]oxazine-1-carboxylate (483.65 mg; 1.47 mmol) in 1,4-dioxane (15.00 ml) were added Pd-PEPPSI-IPentCl 2-methylpyridine (o-picoline) (64.97 mg; 0.07 mmol) and Cs₂CO₃ (503.89 mg; 1.47 mmol) at room temperature. The mixture was stirred for 1 h at room temperature. The mixture was stirred for 2 h at 100° C. The resulting mixture was concentrated under reduced pressure and purified by silica gel column chromatography, eluted with PE/EtOAc (1:1) to afford tert-butyl 7-(2-{[4-(2-methanesulfonylethyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (320.00 mg; 65.7%) as a yellow solid.

N-[4-(2-methanesulfonylethyl)phenyl]-7-{8-methyl-1H,2H,3H-pyrido[2,3-b][1,4]oxazin-7-yl}-5H,6H,7H,8H-pyrido[3,4-d]pyrimidin-2-amine: A mixture of tert-butyl 7-(2-{[4-(2-methanesulfonylethyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (260.00 mg; 0.39 mmol) and TFA (2.00 ml) in DCM (10.00 ml) was stirred for 1 h at room temperature. The resulting mixture was concentrated under reduced pressure. The residue was purified by Prep-HPLC (Column: XBridge Shield RP18 OBD Column, 30*150 mm, 5 um; Mobile Phase A: Water (10 mmol NH₄HCO₃+0.05% NH₄OH), Mobile Phase B: ACN; Flow rate: 60 mL/min; Gradient: 23% B to 53% B in 9 min, Wavelength: 254 nm; RT1 (min): 7; N-[4-(2-methanesulfonylethyl)phenyl]-7-{8-methyl-1H,2H,3H-pyrido[2,3-b][1,4]oxazin-7-yl}-5H,6H,7H,8H-pyrido[3,4-d]pyrimidin-2-amine (56.00 mg; 28.3%) as a yellow solid.

HPLC: 95.76% purity, RT=2.77 min. MS: m/z=481.10 [M+H]⁺. ¹H NMR (300 MHz, DMSO-d6) δ: 9.46 (s, 1H), 8.32 (s, 1H), 7.70 (d, J=8.2 Hz, 2H), 7.29 (s, 1H), 7.18 (d, J=8.2 Hz, 2H), 5.51 (s, 1H), 4.20 (t, J=4.2 Hz, 2H), 3.96 (s, 2H), 3.44-3.35 (m, 2H), 3.30 (d, J=3.9 Hz, 2H), 3.12 (t, J=5.5 Hz, 2H), 2.95-2.85 (m, 5H), 2.79 (t, J=5.7 Hz, 2H), 2.05 (s, 3H).

### Example 162: Synthesis of Compound 2-(2-methoxyethoxy)-N-{2-methyl-4-[(7-{8-methyl-1H,2H,3H-pyrido[2,3-b][1,4]oxazin-7-yl}-5H,6H,7H,8H-pyrido[3,4-d]pyrimidin-2-yl) amino]phenyl}acetamide

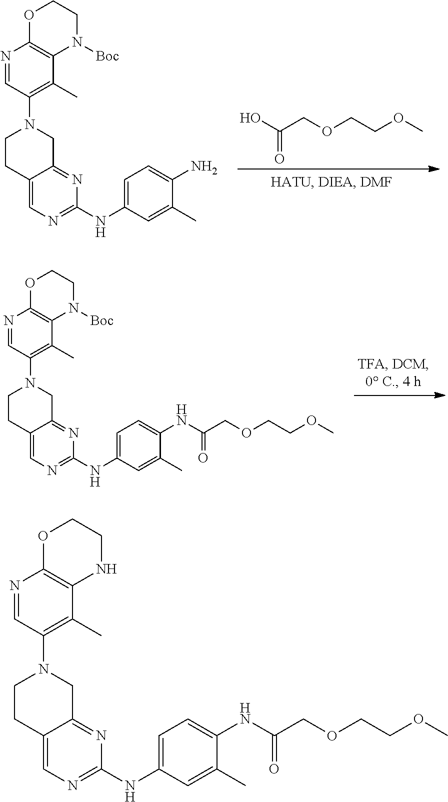

7-[2-({4-[2-(2-methoxyethoxy) acetamido]-3-methylphenyl}amino)-5H,6H,7H,8H-pyrido[3,4-d]pyrimidin-7-yl]-8-methyl-1H,2H,3H-pyrido [2,3-b][1,4]oxazine-1-carboxylate: To a stirred mixture of tert-butyl 7-{2-[(4-amino-3-methylphenyl) amino]-5H,6H,7H,8H-pyrido[3,4-d]pyrimidin-7-yl}-8-methyl-1H,2H,3H-pyrido[2,3-b][1,4]oxazine-1-carboxylate (200.00 mg; 0.32 mmol) and 2-(2-methoxyethoxy) acetic acid (86.72 mg; 0.65 mmol) in DMF (10.00 ml) were added HATU (388.16 mg; 0.97 mmol) and DIEA (87.93 mg; 0.65 mmol) at room temperature. The mixture was stirred for 1 h at room temperature. The resulting mixture was quenched by ice/water and extracted with EtOAc. The combined organic layers were washed with brine, dried over anhydrous Na₂SO₄. After filtration, the filtrate was concentrated under reduced pressure. The resulting mixture was concentrated under reduced pressure and purified by silica gel column chromatography, eluted with PE/EtOAc (1:1) to afford tert-butyl 7-[2-({4-[2-(2-methoxyethoxy) acetamido]-3-methylphenyl}amino)-5H,6H,7H,8H-pyrido[3,4-d]pyrimidin-7-yl]-8-methyl-1H,2H,3H-pyrido[2,3-b][1,4]oxazine-1-carboxylate (200.00 mg; 97.4%) as a brown solid.

2-(2-methoxyethoxy)-N-{2-methyl-4-[(7-{8-methyl-1H,2H,3H-pyrido [2,3-b][1,4]oxazin-7-yl}-5H,6H,7H,8H-pyrido[3,4-d]pyrimidin-2-yl) amino]phenyl}acetamide: A mixture of tert-butyl 7-[2-({4-[2-(2-methoxyethoxy) acetamido]-3-methylphenyl}amino)-5H,6H,7H,8H-pyrido[3,4-d]pyrimidin-7-yl]-8-methyl-1H,2H,3H-pyrido[2,3-b][1,4]oxazine-1-carboxylate (190.00 mg; 0.27 mmol) and TFA (2.00 ml) in DCM (10.00 ml) was stirred for 1 h at room temperature. The resulting mixture was concentrated under reduced pressure. The residue was purified by Prep-HPLC (Column: XBridge Shield RP18 OBD Column, 30*150 mm, 5 μm; Mobile Phase A: Water (10 mmol NH₄HCO₃+0.05% NH₄OH), Mobile Phase B: ACN; Flow rate: 60 mL/min; Gradient: 33% B to 60% B in 9 min; Wavelength: 254 nm; RT1 (min): 7; to afford 2-(2-methoxyethoxy)-N-{2-methyl-4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}acetamide (43.40 mg; 29.9%) as a white solid.

HPLC: 96.85% purity, RT=2.96 min. MS: m/z=520.30 [M+H]⁺. ¹H NMR (300 MHz, DMSO-d6): 9.43 (s, 1H), 8.96 (s, 1H), 8.31 (s, 1H), 7.62-7.53 (m, 2H), 7.36-7.25 (m, 2H), 5.51 (s, 1H), 4.18 (t, J=4.3 Hz, 2H), 4.06 (s, 2H), 3.94 (s, 2H), 3.68 (dd, J=5.8, 3.5 Hz, 2H), 3.53 (dd, J=5.7, 3.4 Hz, 2H), 3.34-3.25 (m, 5H), 3.11 (s, 2H), 2.77 (t, J=5.6 Hz, 2H), 2.14 (s, 3H), 2.03 (s, 3H).

### Example 163: Synthesis of Compound 1-(azetidin-1-yl)-2-(4-((7-(8-methyl-2,3-dihydro-1H-pyrido[2,3-b][1,4]oxazin-7-yl)-5,6,7,8-tetrahydropyrido[3,4-d]pyrimidin-2-yl)amino)phenyl)ethan-1-one

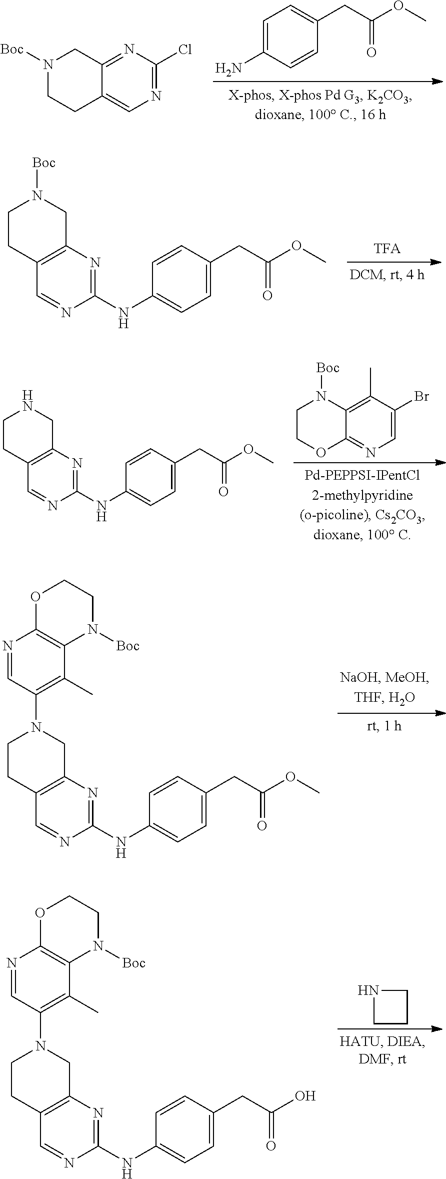

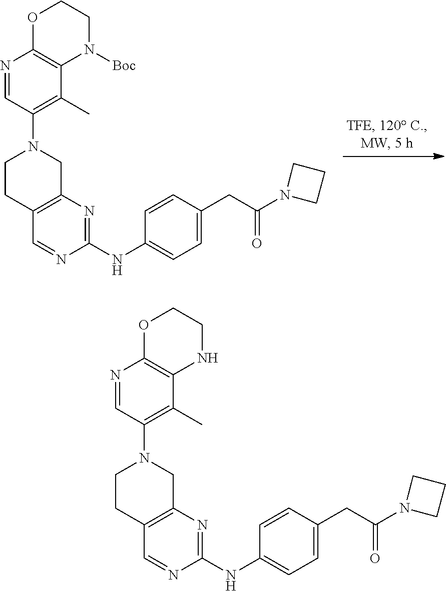 [SMILES: Cc1c(N2CCc3cnc(Nc4ccc(CC(=O)N5CCC5)cc4)nc3C2)cnc2c1N(C(=O)OC(C)(C)C)CCO2 ; Cc1c(N2CCc3cnc(Nc4ccc(CC(=O)N5CCC5)cc4)nc3C2)cnc2c1NCCO2]

2-{[4-(2-methoxy-2-oxoethyl)phenyl]amino}-5H,6H,7H,8H-pyrido[3,4-d]pyrimidine-7-carboxylate: To a stirred mixture of tert-butyl 2-chloro-5H,6H,7H,8H-pyrido[3,4-d]pyrimidine-7-carboxylate (5 g; 18.06 mmol) and methyl 2-(4-aminophenyl)acetate (3.69 g; 21.67 mmol) in 1,4-dioxane (100 ml) were added X-PHOS (1.81 g; 3.61 mmol), K₂CO₃ (5.25 g; 36.12 mmol) and XPhos Pd G3 (1.61 g; 1.81 mmol) in portions at 25 degrees C. The resulting mixture was stirred for 2 h at 100 degrees C. under nitrogen atmosphere. The resulting mixture was concentrated under vacuum. The residue was purified by silica gel column chromatography, eluted with DCM:MeOH=10:1 to afford tert-butyl 2-{[4-(2-methoxy-2-oxoethyl)phenyl]amino}-5H,6H,7H,8H-pyrido[3,4-d]pyrimidine-7-carboxylate (5.28 g; 70.4%) as a yellow solid.

methyl 2-[4-({5H,6H,7H,8H-pyrido[3,4-d]pyrimidin-2-yl}amino)phenyl]acetate: To a stirred mixture of tert-butyl 2-{[4-(2-methoxy-2-oxoethyl)phenyl]amino}-5H,6H,7H,8H-pyrido[3,4-d]pyrimidine-7-carboxylate (5.18 g; 13.00 mmol) in DCM (50 ml) was added TFA (15 ml; 201.94 mmol) in portions at 25 degrees C. The resulting mixture was stirred for 2 h at 25 degrees C. under nitrogen atmosphere. The resulting mixture was concentrated under reduced pressure and purified by silica gel column chromatography, eluted with CH₂Cl₂/MeOH (1:1) to afford methyl 2-[4-({5H,6H,7H,8H-pyrido[3,4-d]pyrimidin-2-yl}amino)phenyl]acetate (3.10 g; 78.6%) as a pink solid.

7-(2-{[4-(2-methoxy-2-oxoethyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate: To a stirred mixture of tert-butyl 7-bromo-8-methyl-1H,2H,3H-pyrido[2,3-b][1,4]oxazine-1-carboxylate (4.20 g; 12.76 mmol) and methyl 2-[4-({5H,6H,7H,8H-pyrido[3,4-d]pyrimidin-2-yl}amino)phenyl]acetate (3.50 g; 11.50 mmol) in Dioxane-1,4 (80 ml) were added Pd-PEPPSI-IPentCl 2-methylpyridine (o-picoline) (0.30 g; 0.33 mmol) and Cs₂CO₃ (7.89 g; 22.99 mmol) at room temperature. The mixture was stirred for 12 h at 100 degrees C. under argon atmosphere. The resulting mixture was concentrated under reduced pressure and purified by silica gel column chromatography, eluted with PE/EtOAc (1:1) to afford tert-butyl 7-(2-{[4-(2-methoxy-2-oxoethyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (4.31 g; 51.9%) as a yellow oil.

2-{4-[(7-{1-[(tert-butoxy)carbonyl]-8-methyl-1H,2H,3H-pyrido[2,3-b][1,4]oxazin-7-yl}-5H,6H,7H,8H-pyrido[3,4-d]pyrimidin-2-yl)amino]phenyl}acetic acid: To a stirred solution of tert-butyl 7-(2-{[4-(2-methoxy-2-oxoethyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (4.31 g; 7.13 mmol) in THF (40 ml) and H₂O (20 ml) and MeOH (40 ml) was added NaOH (1.30 g; 30.88 mmol) in portions at room temperature. The mixture was stirred for 3 h at 50 degrees C. The solvent was removed under vacuum, and 20 ml water was added to the residue, and adjust PH=6-7 with Conc. HCl, the solid was collected by filtration, washed with water, dried under vacuum. This resulted in 2-{4-[(7-{1-[(tert-butoxy)carbonyl]-8-methyl-1H,2H,3H-pyrido[2,3-b][1,4]oxazin-7-yl}-5H,6H,7H,8H-pyrido[3,4-d]pyrimidin-2-yl)amino]phenyl}acetic acid (3.80 g; Crude Product) as a yellow solid.

7-[2-({4-[2-(azetidin-1-yl)-2-oxoethyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate: To a stirred mixture of 2-{4-[(7-{1-[(tert-butoxy)carbonyl]-8-methyl-1H,2H,3H-pyrido[2,3-b][1,4]oxazin-7-yl}-5H,6H,7H,8H-pyrido[3,4-d]pyrimidin-2-yl)amino]phenyl}acetic acid (200 mg; 0.31 mmol) and in DMF (5 ml) were added HATU (367.03 mg; 0.92 mmol) and DIEA (83.14 mg; 0.61 mmol) at room temperature. The mixture was stirred for 1 h at room temperature. The resulting mixture was quenched by ice/water and extracted with EtOAc. The combined organic layers were washed with brine, dried over anhydrous Na₂SO₄. After filtration, the filtrate was concentrated under reduced pressure. The resulting mixture was concentrated under reduced pressure and purified by silica gel column chromatography, eluted with CH₂Cl₂/MeOH (1:1) to afford tert-butyl 7-[2-({4-[2-(azetidin-1-yl)-2-oxoethyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate (120 mg; 67.0%) as a yellow solid.

1-(azetidin-1-yl)-2-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}ethan-1-one: A solution of tert-butyl 7-[2-({4-[2-(azetidin-1-yl)-2-oxoethyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate (190 mg; 0.29 mmol) in 2,2,2-trifluoroethan-1-ol (1 ml) was stirred for 5 h under MW at 120 degrees C. The resulting mixture was concentrated under reduced pressure. The residue was purified by Prep-HPLC(Column: XBridge Prep OBD C18 Column, 30*150 mm, 5 μm; Mobile Phase A: 10 mmol NH₄HCO₃+0.05% NH₄OH, Mobile Phase B: ACN; Flow rate: 60 mL/min; Gradient: 20% B to 50% B in 9 min; Wavelength: 254 nm; RT1 (min): 7; Number Of Runs: 2) 1-(azetidin-1-yl)-2-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}ethan-1-one (48.2 mg; 34.7%) as a pink solid.

HPLC 99.31% purity, 3.80 min, MS: m/s=472.25 [M+H]⁺. ¹H NMR (300 MHz, DMSO-d6): 9.45 (s, 1H), 8.30 (s, 1H), 7.64 (d, J=8.2 Hz, 2H), 7.27 (s, 1H), 7.09 (d, J=8.2 Hz, 2H), 5.51 (s, 1H), 4.14 (dd, J=14.6, 6.6 Hz, 4H), 3.94 (s, 2H), 3.81 (t, J=7.7 Hz, 2H), 3.23 (s, 4H), 3.10 (s, 2H), 2.77 (s, 2H), 2.15 (p, J=7.7 Hz, 2H), 2.03 (s, 3H).

### Example 164: Synthesis of Compound 2-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}-1-{2-oxa-6-azaspiro[3.3]heptan-6-yl}ethan-1-one

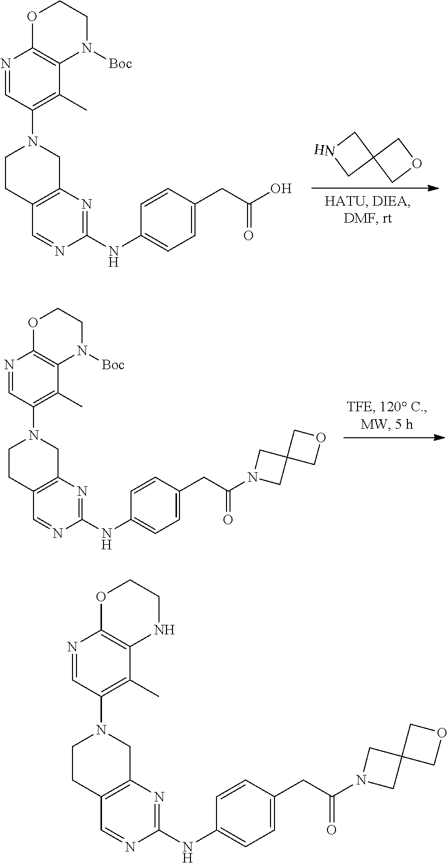 [SMILES: Cc1c(N2CCc3cnc(Nc4ccc(CC(=O)O)cc4)nc3C2)cnc2c1N(C(=O)OC(C)(C)C)CCO2 ; C1NCC12COC2 ; Cc1c(N2CCc3cnc(Nc4ccc(CC(=O)N5CC6(COC6)C5)cc4)nc3C2)cnc2c1N(C(=O)OC(C)(C)C)CCO2 ; Cc1c(N2CCc3cnc(Nc4ccc(CC(=O)N5CC6(COC6)C5)cc4)nc3C2)cnc2c1NCCO2]

8-methyl-7-(2-{[4-(2-{2-oxa-6-azaspiro[3.3]heptan-6-yl}-2-oxoethyl)phenyl]amino}-5H,6H,7H,8H-pyrido[3,4-d]pyrimidin-7-yl)-1H,2H,3H-pyrido[2,3-b][1,4]oxazine-1-carboxylate: To a stirred mixture of 2-{4-[(7-{1-[(tert-butoxy)carbonyl]-8-methyl-1H,2H,3H-pyrido[2,3-b][1,4]oxazin-7-yl}-5H,6H,7H,8H-pyrido[3,4-d]pyrimidin-2-yl)amino]phenyl}acetic acid (200 mg; 0.31 mmol) and in DMF (5 ml) were added HATU (367.03 mg; 0.92 mmol) and DIEA (83.14 mg; 0.61 mmol) at room temperature. The mixture was stirred for 1 h at room temperature. The resulting mixture was quenched by ice/water and extracted with EtOAc. The combined organic layers were washed with brine, dried over anhydrous Na₂SO₄. After filtration, the filtrate was concentrated under reduced pressure. The resulting mixture was concentrated under reduced pressure and purified by silica gel column chromatography, eluted with CH₂Cl₂/MeOH (1:1) to afford tert-butyl 8-methyl-7-(2-{[4-(2-{2-oxa-6-azaspiro[3.3]heptan-6-yl}-2-oxoethyl)phenyl]amino}-5H,6H,7H,8H-pyrido[3,4-d]pyrimidin-7-yl)-1H,2H,3H-pyrido[2,3-b][1,4]oxazine-1-carboxylate (150 mg; 78.0%) as a yellow solid.

2-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}-1-{2-oxa-6-azaspiro[3.3]heptan-6-yl}ethan-1-one: A solution of tert-butyl 8-methyl-7-(2-{[4-(2-{2-oxa-6-azaspiro[3.3]heptan-6-yl}-2-oxoethyl)phenyl]amino}-5H,6H,7H,8H-pyrido[3,4-d]pyrimidin-7-yl)-1H,2H,3H-pyrido[2,3-b][1,4]oxazine-1-carboxylate (190 mg; 0.27 mmol) in 2,2,2-trifluoroethan-1-ol (10 ml) was stirred for 5 h under MW at 120 degrees C. The resulting mixture was concentrated under reduced pressure. The residue was purified by Prep-HPLC(Column: Xselect CSH C18 OBD Column 30*150 mm 5 μm, n; Mobile Phase A: Water (0.1% FA), Mobile Phase B: ACN; Flow rate: 60 mL/min; Gradient: 5% B to 27% B in 8 min, Wavelength: 254/220 nm; RT1 (min): 6.7; Number Of Runs: 2) 2-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}-1-{2-oxa-6-azaspiro[3.3]heptan-6-yl}ethan-1-one (79.70 mg; 56.7%) as a grey solid.

HPLC 99.76% purity, 3.58 min, MS: m/s=514.30 [M+H]⁺. ¹H NMR (300 MHz, DMSO-d6): 9.45 (s, 1H), 8.30 (s, 1H), 7.64 (d, J=8.5 Hz, 2H), 7.27 (s, 1H), 7.07 (d, J=8.4 Hz, 2H), 5.51 (s, 1H), 4.63 (d, J=1.3 Hz, 4H), 4.31 (s, 2H), 4.17 (d, J=4.8 Hz, 2H), 4.01 (s, 2H), 3.98 (s, 2H), 3.96 (d, J=17.5 Hz, 4H), 3.10 (s, 2H), 2.77 (s, 2H), 2.04 (d, J=11.7 Hz, 3H).

### Example 165 synthesis of compound 2-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}-1-[3-(morpholin-4-yl)azetidin-1-yl]ethan-1-one

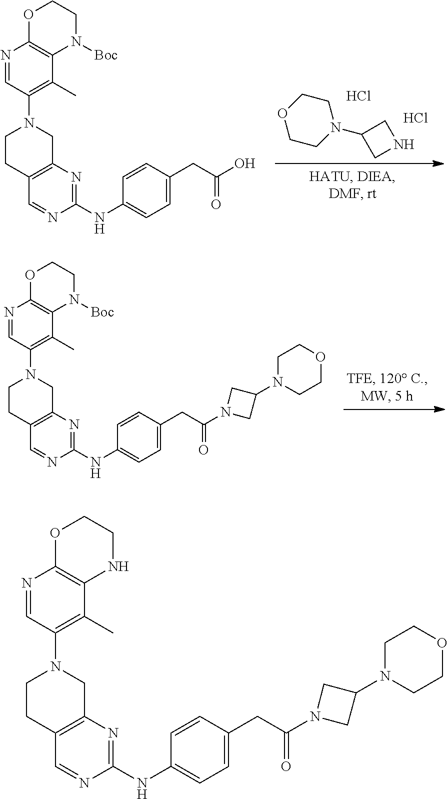 [SMILES: Cc1c(N2CCc3cnc(Nc4ccc(CC(=O)O)cc4)nc3C2)cnc2c1N(C(=O)OC(C)(C)C)CCO2 ; C1CN(C2CNC2)CCO1 ; Cl ; Cl ; Cc1c(N2CCc3cnc(Nc4ccc(CC(=O)N5CC(N6CCOCC6)C5)cc4)nc3C2)cnc2c1N(C(=O)OC(C)(C)C)CCO2 ; Cc1c(N2CCc3cnc(Nc4ccc(CC(=O)N5CC(N6CCOCC6)C5)cc4)nc3C2)cnc2c1NCCO2]

8-methyl-7-{2-[(4-{2-[3-(morpholin-4-yl)azetidin-1-yl]-2-oxoethyl}phenyl)amino]-5H,6H,7H,8H-pyrido[3,4-d]pyrimidin-7-yl}-1H,2H,3H-pyrido[2,3-b][1,4]oxazine-1-carboxylate: To a stirred mixture of 2-{4-[(7-{1-[(tert-butoxy)carbonyl]-8-methyl-1H,2H,3H-pyrido[2,3-b][1,4]oxazin-7-yl}-5H,6H,7H,8H-pyrido[3,4-d]pyrimidin-2-yl)amino]phenyl}acetic acid (200 mg; 0.31 mmol) and in DMF (5 ml) were added HATU (367.03 mg; 0.92 mmol) and DIEA (83.14 mg; 0.61 mmol) at room temperature. The mixture was stirred for 1 h at room temperature. The resulting mixture was quenched by ice/water and extracted with EtOAc. The combined organic layers were washed with brine, dried over anhydrous Na₂SO₄. After filtration, the filtrate was concentrated under reduced pressure. The resulting mixture was concentrated under reduced pressure and purified by silica gel column chromatography, eluted with CH₂Cl₂/MeOH (1:1) to afford tert-butyl 8-methyl-7-{2-[(4-{2-[3-(morpholin-4-yl)azetidin-1-yl]-2-oxoethyl}phenyl)amino]-5H,6H,7H,8H-pyrido[3,4-d]pyrimidin-7-yl}-1H,2H,3H-pyrido[2,3-b][1,4]oxazine-1-carboxylate (190 mg; 90.4%) as a brown solid.

2-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}-1-[3-(morpholin-4-yl)azetidin-1-yl]ethan-1-one: A solution of tert-butyl 8-methyl-7-{2-[(4-{2-[3-(morpholin-4-yl)azetidin-1-yl]-2-oxoethyl}phenyl)amino]-5H,6H,7H,8H-pyrido[3,4-d]pyrimidin-7-yl}-1H,2H,3H-pyrido[2,3-b][1,4]oxazine-1-carboxylate (180.00 mg; 0.24 mmol) in 2,2,2-trifluoroethan-1-ol (10 ml) was stirred for 5 h at 120 degrees C. The resulting mixture was concentrated under reduced pressure. The residue was purified by Prep-HPLC(Column: XBridge Prep OBD C18 Column, 30*150 mm, 5 μm; Mobile Phase A: 10 mmol NH₄HCO₃+0.05% NH₄OH, Mobile Phase B: ACN; Flow rate: 60 mL/min; Gradient: 18% B to 48% B in 9 min, Wavelength: 254 nm; RT1 (min): 7; Number Of Runs: 0) 2-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}-1-[3-(morpholin-4-yl)azetidin-1-yl]ethan-1-one (67.00 mg; 0.12 mmol; 49.5%; pink solid; Purified Product)

HPLC 99.30% purity, 3.07 min, MS: m/s=557.35[M+H]⁺. ¹H NMR (300 MHz, DMSO-d6): 9.47 (s, 1H), 8.33 (s, 1H), 7.67 (d, J=8.5 Hz, 2H), 7.29 (s, 1H), 7.11 (d, J=8.4 Hz, 2H), 5.53 (s, 1H), 4.19 (s, 3H), 4.15 (d, J=7.7 Hz, 3H), 3.86 (s, 1H), 3.66 (dd, J=9.9, 5.0 Hz, 1H), 3.59 (t, J=4.5 Hz, 4H), 3.39 (dd, J=14.1, 6.0 Hz, 2H), 3.23 (s, 2H), 3.12 (s, 3H), 2.79 (s, 2H), 2.29 (s, 4H), 2.05 (s, 3H).

### Example 168 synthesis of compound imino(methyl)(3-((7-(8-methyl-2,3-dihydro-1H-pyrido[2,3-b][1,4]oxazin-7-yl)-5,6,7,8-tetrahydropyrido[3,4-d]pyrimidin-2-yl)amino)benzyl)-16-sulfanone

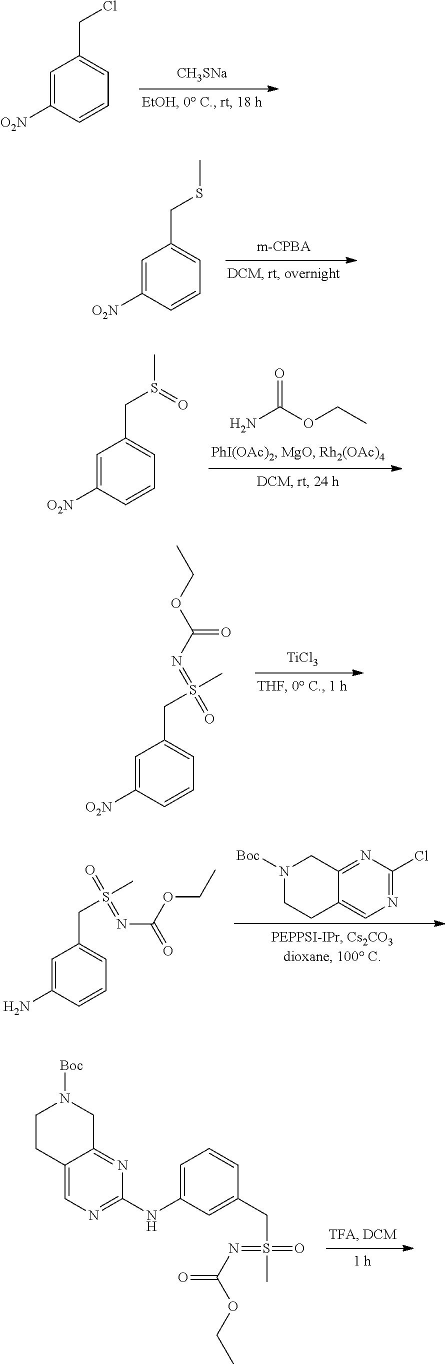

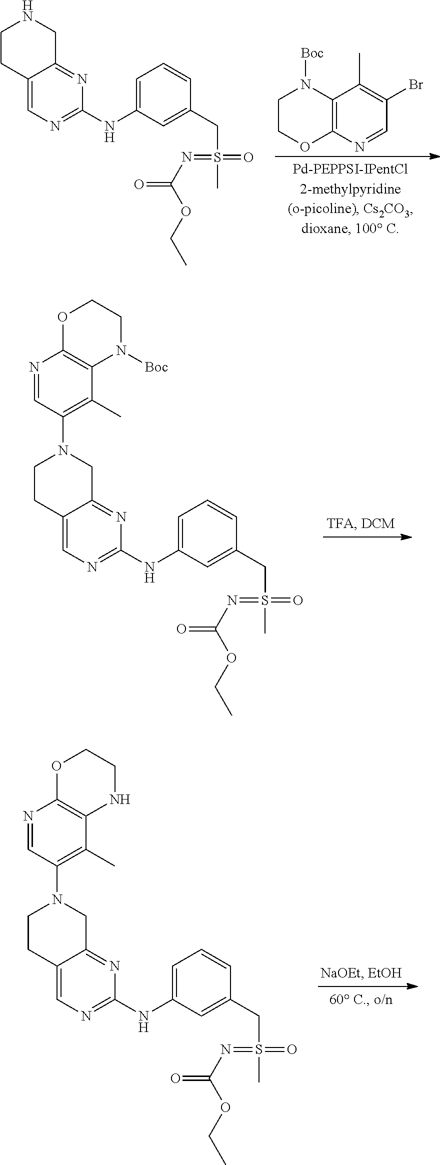

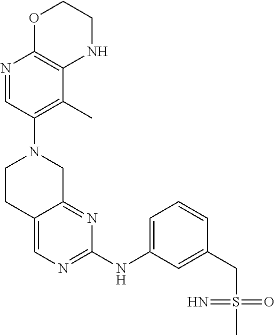 [SMILES: Cc1c(N2CCc3cnc(Nc4cccc(CS(C)(=N)=O)c4)nc3C2)cnc2c1NCCO2]

1-[(methylsulfanyl)methyl]-3-nitrobenzene: A solution of 1-(chloromethyl)-3-nitrobenzene (5 g; 27.68 mmol) and (methyl sulfanyl)sodium (3 g; 40.66 mmol) in EtOH (50 ml) was stirred for overnight at room temperature under nitrogen atmosphere. The resulting mixture was quenched by ice/water and extracted with EtOAc. The combined organic layers were washed with brine, dried over anhydrous Na₂SO₄. After filtration, the filtrate was concentrated under reduced pressure. This resulted in 1-[(methylsulfanyl)methyl]-3-nitrobenzene (4.70 g; Crude Product) as a white solid.

1-(methanesulfinylmethyl)-3-nitrobenzene: To a stirred solution of 1-[(methylsulfanyl)methyl]-3-nitrobenzene (9.50 g; 49.26 mmol) in DCM (100 ml) was added mCPBA (8.95 g; 49.26 mmol) dropwise slowly at 0 degree C. under N₂ atmosphere. The resulting mixture was stirred for two days at room temperature under N₂ atmosphere. The resulting mixture was added water. then added saturated Na₂S₂O₃, extracted with DCM (3×5 0 mL). The combined organic layers were washed with brine (15 mL), dried over anhydrous Na₂SO₄. The resulting mixture was concentrated under reduced pressure. The residue was purified by silica gel column, eluted with 100% EtOAc in PE to afford 1-(methanesulfinylmethyl)-3-nitrobenzene (5.70 g; 55.8%) as a white yellow solid.

ethyl (methyl(3-nitrobenzyl)(oxo)-16-sulfaneylidene)carbamate: To a stirred solution of 1-(methanesulfinylmethyl)-3-nitrobenzene (1 g; 4.52 mmol) in DCM (15 ml) were added ethyl carbamate (0.64 g; 6.78 mmol), Iodobenzene diacetate (6.13 g; 18.07 mmol), Rh₂(OAc)₄ (0.11 g; 0.23 mmol) and MgO (0.38 g; 9.03 mmol) in portions at room temperature under N₂ atmosphere. The resulting mixture was stirred for overnight at room temperature under N₂ atmosphere. The residue was purified by silica gel column eluted with 75% EtOAc in PE to afford ethyl (methyl(3-nitrobenzyl)(oxo)-sulfaneylidene)carbamate (1.20 g; 96.2%) as a green oil.

ethyl ((3-aminobenzyl)(methyl)(oxo)-16-sulfaneylidene)carbamate: To a stirred solution of ethyl (methyl(3-nitrobenzyl)(oxo)-16-sulfaneylidene)carbamate (1.50 g; 4.72 mmol) in THF (50 ml) was dropwise TiCl3 (15-20% w/v solution in 2N HCl) (30 ml) in portions at 0 degree C. under N₂ atmosphere. The resulting mixture was stirred for 1 h at 0 degree C. under N₂ atmosphere. The resulting mixture was diluted with water (15 mL), then added NaOH (1 mol/L) to adjust the pH to 10 and extracted with EtOAc (3×100 mL). The combined organic layers were washed with brine (300 mL). The resulting mixture was concentrated under reduced pressure to afford ethyl ethyl ((3-aminobenzyl)(methyl)(oxo)-sulfaneylidene)carbamate (900 mg; 64.0%) as a white oil.

tert-butyl tert-butyl 2-((3-((N-(ethoxycarbonyl)-S-methylsulfonimidoyl)methyl)phenyl)amino)-5,8-dihydropyrido[3,4-d]pyrimidine-7(6H)-carboxylate: To a stirred solution of ethyl ethyl ((3-aminobenzyl)(methyl)(oxo)-sulfaneylidene)carbamate (800 mg; 2.97 mmol) in 1,4-dioxane (10 ml) were added tert-butyl 2-chloro-5H,6H,7H,8H-pyrido[3,4-d]pyrimidine-7-carboxylate (841.84 mg; 2.97 mmol), PEPPSI-IPr (193.03 mg; 0.30 mmol) and Cs₂CO₃ (3050.72 mg; 8.90 mmol) in portions at room temperature under N₂ atmosphere. The resulting mixture was stirred for 1 h at 100 degree C. under N₂ atmosphere. The residue was purified by silica gel column eluted with 100% EtOAc in PE to afford crude product. The crude product was repurified by C18 flash column eluted with 80% ACN in Water to afford tert-butyl tert-butyl 2-((3-((N-(ethoxycarbonyl)-S-methylsulfonimidoyl)methyl)phenyl)amino)-5,8-dihydropyrido[3,4-d]pyrimidine-7(6H)-carboxylate (410 mg; 27.3%) as a white solid.

ethyl N-[methyl(oxo){[3-({5H,6H,7H,8H-pyrido[3,4-d]pyrimidin-2-yl}amino)phenyl]methyl}-6-sulfanylidene]carbamate: To a stirred solution of tert-butyl 2-((3-((N-(ethoxycarbonyl)-S-methylsulfonimidoyl)methyl)phenyl)amino)-5,8-dihydropyrido[3,4-d]pyrimidine-7(6H)-carboxylate (390 mg; 0.72 mmol) in DCM (3 ml) was added TFA (2 ml) in portions at 0 degree C. under N₂ atmosphere. The resulting mixture was stirred for 1 h at room temperature. The resulting mixture was diluted with water (20 mL), adjust PH=10 by saturated NaHCO₃, extracted with DCM (3×50 mL). The combined organic layers were washed with brine (15 mL) and dried over anhydrous Na₂SO₄. The resulting mixture was concentrated under reduced pressure. The residue was purified by C18 flash column eluted with 70% ACN in water to afford ethyl N-[methyl(oxo){[3-({5H,6H,7H,8H-pyrido[3,4-d]pyrimidin-2-yl}amino)phenyl]methyl}-6-sulfanylidene]carbamate (150 mg; 48.7%) as a yellow solid.

tert-butyl 7-(2-{[3-({[(ethoxycarbonyl)imino](methyl)oxo-6-sulfanyl}methyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate: To a stirred solution of ethyl N-[methyl(oxo){[3-({5H,6H,7H,8H-pyrido[3,4-d]pyrimidin-2-yl}amino)phenyl]methyl}-6-sulfanylidene]carbamate (130 mg; 0.32 mmol) in 1,4-dioxane (3 ml) were added tert-butyl 7-bromo-8-methyl-1H,2H,3H-pyrido[2,3-b][1,4]oxazine-1-carboxylate (109.88 mg; 0.32 mmol), PEPPSI-IPr (20.64 mg; 0.03 mmol) and Cs₂CO₃ (326.26 mg; 0.95 mmol) in portions at room temperature under N₂ atmosphere. The resulting mixture was stirred for overnight at 100 degree C. under N₂. The residue was purified by silica gel column and eluted with 100% EtOAc in PE to afford tert-butyl 7-(2-{[3-({[(ethoxycarbonyl)imino](methyl)oxo-6-sulfanyl}methyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (160 mg; 67.4) as a yellow solid.

ethyl N-[methyl({3-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}methyl)oxo-6-sulfanylidene]carbamate: To a stirred solution of tert-butyl 7-(2-{[3-({[(ethoxycarbonyl)imino](methyl)oxo-6-sulfanyl}methyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (140 mg; 0.20 mmol) in DCM (1.80 ml) was added TFA (0.60 ml) in portions at 0 degree C. under N₂ atmosphere. The resulting mixture was stirred for 1 h at room temperature under N₂. The resulting mixture was diluted with water (15 mL), then added NaHCO₃ and extracted with DCM (3×30 mL). The combined organic layers were washed with brine (50 mL), dried over anhydrous Na₂SO₄. The resulting mixture was concentrated under reduced pressure. The residue was purified by silica gel column, eluted with 30% EtOAc in PE to afford ethyl N-[methyl({3-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}methyl)oxo-6-sulfanylidene]carbamate (35 mg; 19.4%) as a yellow solid.

imino(methyl)(3-((7-(8-methyl-2,3-dihydro-1H-pyrido[2,3-b][1,4]oxazin-7-yl)-5,6,7,8-tetrahydropyrido[3,4-d]pyrimidin-2-yl)amino)benzyl)-16-sulfanone: To a stirred solution of ethyl N-[methyl({3-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}methyl)oxo-6-sulfanylidene]carbamate (100 mg; 0.07 mmol) in EtOH (3 ml) was added EtONa/EtOH (w/w 21%) (0.17 ml; 0.77 mmol) in portions at 0 degree C. under N₂ atmosphere. The resulting mixture was stirred for 48 h at 60 degree C. under N₂. The residue was purified by Pre-HPLC to afford imino(methyl)(3-((7-(8-methyl-2,3-dihydro-1H-pyrido[2,3-b][1,4]oxazin-7-yl)-5,6,7,8-tetrahydropyrido[3,4-d]pyrimidin-2-yl)amino)benzyl)-16-sulfanone (15.70 mg; 49.2%) as a white solid.

HPLC 95.02% purity, 2.280 min, MS: m/s=466.20 [M+H]⁺. ¹H NMR (400 MHz, DMSO-d6): 9.56 (s, 1H), 8.33 (s, 1H), 7.85 (s, 1H), 7.64 (d, J=8.2 Hz, 1H), 7.31-7.21 (m, 2H), 6.97 (d, J=7.5 Hz, 1H), 5.53 (s, 1H), 4.37-4.25 (m, 2H), 4.19 (t, J=4.4 Hz, 2H), 3.96 (s, 2H), 3.53 (s, 2H), 3.12 (t, J=5.7 Hz, 2H), 2.81 (d, J=13.7 Hz, 5H), 2.04 (s, 3H).

### Example 169 & 170 synthesis of compound (R)-2-(4-((6-(8-methyl-2,3-dihydro-1H-pyrido[2,3-b][1,4]oxazin-7-yl)-5,6,7,8-tetrahydro-2,6-naphthyridin-3-yl)amino)phenyl)tetrahydrothiophene 1,1-dioxide and (S)-2-(4-((6-(8-methyl-2,3-dihydro-1H-pyrido[2,3-b][1,4]oxazin-7-yl)-5,6,7,8-tetrahydro-2,6-naphthyridin-3-yl)amino)phenyl)tetrahydrothiophene 1,1-dioxide

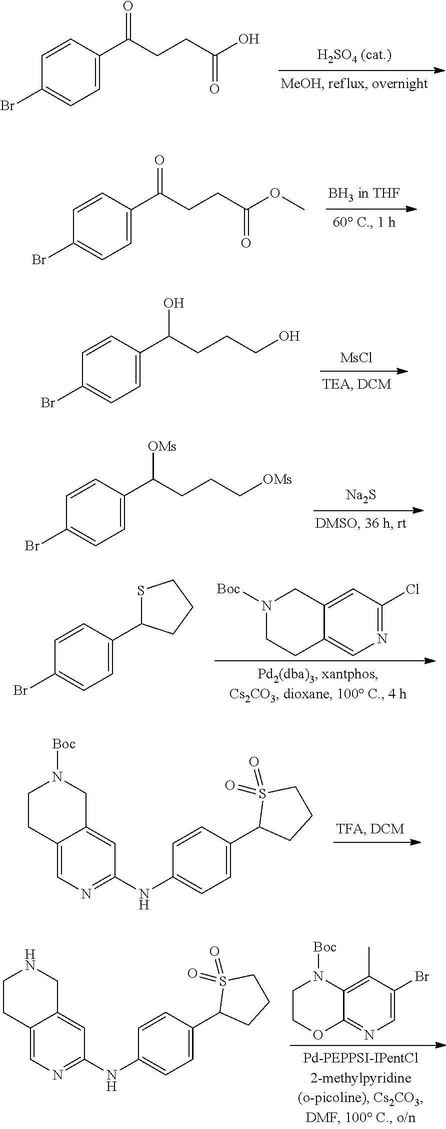

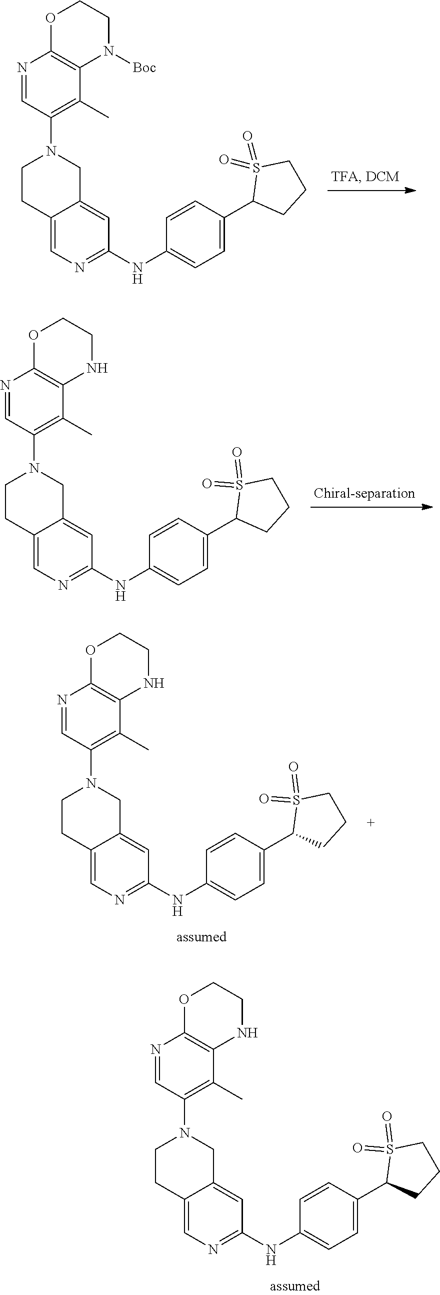 [SMILES: Cc1c(N2CCc3cnc(Nc4ccc(C5CCCS5(=O)=O)cc4)cc3C2)cnc2c1N(C(=O)OC(C)(C)C)CCO2 ; Cc1c(N2CCc3cnc(Nc4ccc(C5CCCS5(=O)=O)cc4)cc3C2)cnc2c1NCCO2 ; Cc1c(N2CCc3cnc(Nc4ccc([C@H]5CCCS5(=O)=O)cc4)cc3C2)cnc2c1NCCO2 ; Cc1c(N2CCc3cnc(Nc4ccc([C@@H]5CCCS5(=O)=O)cc4)cc3C2)cnc2c1NCCO2]

methyl 4-(4-bromophenyl)-4-oxobutanoate: To a stirred solution of 4-(4-bromophenyl)-4-oxobutanoic acid (9 g; 33.26 mmol) in MeOH (180 ml) was added H₂SO₄ (9 ml; 108.00 mmol) drop wised at room temperature under N₂ atmosphere.

The resulting mixture was stirred for overnight at 70 degrees C. The reaction was concentrated under vacuum to afford methyl 4-(4-bromophenyl)-4-oxobutanoate (9 g; Crude Product) as a white solid.

1-(4-bromophenyl)butane-1,4-diol: A solution of methyl 4-(4-bromophenyl)-4-oxobutanoate (4.80 g; 16.64 mmol) in BH₃ in THF (20 ml; 20.00 mmol) was stirred for 4 h at 60 degree C. under N₂ atmosphere. The reaction was quenched by MeOH and concentrated under reduced pressure. The residue (with the previous batch) was purified by silica gel column eluted with 40% EA in PE to afford 1-(4-bromophenyl)butane-1,4-diol (8 g; 96.11%) as a white solid.

1-(4-bromophenyl)-4-(methanesulfonyloxy)butyl methanesulfonate: To a stirred solution of 1-(4-bromophenyl)butane-1,4-diol (6 g; 24.48 mmol) and triethylamine (28.35 ml; 195.83 mmol) in DCM (120 ml) was added MsCl (9.97 ml; 122.39 mmol) dropwise at room temperature under N₂ atmosphere. The resulting mixture was stirred for overnight at 25 degree C. under N₂ atmosphere. The reaction was quenched with H₂O and extracted with DCM to afford 1-(4-bromophenyl)-4-(methanesulfonyloxy)butyl methanesulfonate (7 g; Crude Product) as a colorless oil.

2-(4-bromophenyl)tetrahydrothiophene: To a stirred solution of 1-(4-bromophenyl)-4-(methanesulfonyloxy)butyl methanesulfonate (7 g; 12.21 mmol.) in DMSO (200 ml) was added sodium sulfide (5.02 g; 61.05 mmol) in portions at room temperature under N₂ atmosphere. The resulting mixture was stirred for overnight at 25 degree C. under N₂ atmosphere. The resulting mixture was poured into ice-cold water and extracted with EA. The combined organic layers were concentrated under reduced pressure. The residue with purified by silica gel column with the previous batch eluted with 30% EA in PE to afford 2-(4-bromophenyl)tetrahydrothiophene (3.70 g; 87.60%) as a yellow solid.

tert-butyl 7-(7-((4-(1,1-dioxidotetrahydrothiophen-2-yl)phenyl)amino)-3,4-dihydro-2,6-naphthyridin-2(1H)-yl)-8-methyl-2,3-dihydro-1H-pyrido[2,3-b][1,4]oxazine-1-carboxylate: To a stirred solution of 2-(4-((5,6,7,8-tetrahydro-2,6-naphthyridin-3-yl)amino)phenyl)tetrahydrothiophene 1,1-dioxide (800 mg; 3.79 mmol) and tert-butyl 7-chloro-1,2,3,4-tetrahydro-2,6-naphthyridine-2-carboxylate (800 mg; 2.83 mmol) in Dioxane-1,4 (5 ml) was added Cs₂CO₃ (2597.23 mg; 7.57 mmol), t-BuBrettPhos Pd G3 (340.95 mg; 0.38 mmol) and t-BuBrettPhos (193.18 mg; 0.38 mmol) in portions at room temperature under N₂ atmosphere. The resulting mixture was stirred for 16 h at 100 degree C. under N₂ atmosphere. The resulting mixture was concentrated under reduced pressure. The residue was purified by silica gel column eluted with 90% EA in PE to afford tert-butyl 7-(7-((4-(1,1-dioxidotetrahydrothiophen-2-yl)phenyl)amino)-3,4-dihydro-2,6-naphthyridin-2(1H)-yl)-8-methyl-2,3-dihydro-1H-pyrido[2,3-b][1,4]oxazine-1-carboxylate (700 mg; 38.12%) as a yellow solid.

2-(4-((6-(8-methyl-2,3-dihydro-1H-pyrido[2,3-b][1,4]oxazin-7-yl)-5,6,7,8-tetrahydro-2,6-naphthyridin-3-yl)amino)phenyl)tetrahydrothiophene 1,1-dioxide: To a stirred solution of tert-butyl 7-(7-((4-(1,1-dioxidotetrahydrothiophen-2-yl)phenyl)amino)-3,4-dihydro-2,6-naphthyridin-2(1H)-yl)-8-methyl-2,3-dihydro-1H-pyrido[2,3-b][1,4]oxazine-1-carboxylate (690 mg; 1.42 mmol) in DCM (3 ml) was added TFA (1 ml) in portion at room temperature. The resulting mixture was stirred for 2 hours at room temperature. The resulting mixture was concentrated under reduced pressure. The residue was purified by C18 flash column eluted with 28% ACN in water (0.5 mmol NH₄HCO₃) to afford 2-(4-((6-(8-methyl-2,3-dihydro-1H-pyrido[2,3-b][1,4]oxazin-7-yl)-5,6,7,8-tetrahydro-2,6-naphthyridin-3-yl)amino)phenyl)tetrahydrothiophene 1,1-dioxide (390 mg; 73.29%) as a yellow solid.

tert-butyl 7-(7-((4-(1,1-dioxidotetrahydrothiophen-2-yl)phenyl)amino)-3,4-dihydro-2,6-naphthyridin-2(1H)-yl)-8-methyl-2,3-dihydro-1H-pyrido[2,3-b][1,4]oxazine-1-carboxylate: To a stirred solution of 2-{4-[(5,6,7,8-tetrahydro-2,6-naphthyridin-3-yl)amino]phenyl}-1 lambda6-thiolane-1,1-dione (350 mg; 0.93 mmol) and tert-butyl 7-bromo-8-methyl-1H,2H,3H-pyrido[2,3-b][1,4]oxazine-1-carboxylate (442.75 mg; 1.21 mmol) in DMF (5 ml) was added Cs₂CO₃ (638.70 mg; 1.86 mmol) and Pd-PEPPSI-IPentCl 2-methylpyridine (o-picoline) (82.35 mg; 0.09 mmol) at room temperature. The resulting mixture was stirred for overnight at 100 degree C. under N₂ atmosphere. The resulting mixture was poured into ice-cold water. The solution was extracted with EA. The combined organic layers were combined and concentrated under reduced pressure. The residue was purified by silica gel column eluted with 5% MeOH in DCM to afford tert-butyl 7-(7-((4-(1,1-dioxidotetrahydrothiophen-2-yl)phenyl)amino)-3,4-dihydro-2,6-naphthyridin-2(1H)-yl)-8-methyl-2,3-dihydro-1H-pyrido[2,3-b][1,4]oxazine-1-carboxylate (290 mg; 28.44%) as a yellow solid.

2-(4-((6-(8-methyl-2,3-dihydro-1H-pyrido[2,3-b][1,4]oxazin-7-yl)-5,6,7,8-tetrahydro-2,6-naphthyridin-3-yl)amino)phenyl)tetrahydrothiophene 1,1-dioxide: To a stirred solution of tert-butyl 7-(7-{[4-(1,1-dioxo-1 lambda6-thiolan-2-yl)phenyl]amino}-1,2,3,4-tetrahydro-2,6-naphthyridin-2-yl)-8-methyl-1H,2H,3H-pyrido[2,3-b][1,4]oxazine-1-carboxylate (280 mg; 0.47 mmol) in DCM (3 ml) was added TFA (1 ml) dropwise. The resulting mixture was stirred for 2 hours at room temperature. The resulting mixture was concentrated under reduced pressure. The residue was purified by C18 flash column eluted with 20% ACN in water (0.5 mmol NH₄HCO₃) to afford 2-(4-((6-(8-methyl-2,3-dihydro-1H-pyrido[2,3-b][1,4]oxazin-7-yl)-5,6,7,8-tetrahydro-2,6-naphthyridin-3-yl)amino)phenyl)tetrahydrothiophene 1,1-dioxide (110 mg; 47.29%) as a yellow oil.

(R)-2-(4-((6-(8-methyl-2,3-dihydro-1H-pyrido[2,3-b][1,4]oxazin-7-yl)-5,6,7,8-tetrahydro-2,6-naphthyridin-3-yl)amino)phenyl)tetrahydrothiophene 1,1-dioxide and 2-(4-((6-(8-methyl-2,3-dihydro-1H-pyrido[2,3-b][1,4]oxazin-7-yl)-5,6,7,8-tetrahydro-2,6-naphthyridin-3-yl)amino)phenyl)tetrahydrothiophene 1,1-dioxide: (110 mg; 0.22 mmol) was purified by Prep-Chiral-HPLC with the following condition (Column: (R, R) WHELK-01, 4.6*50 mm, 3.5 μm; Mobile Phase A: MtBE (0.1% DEA): EtOH=50: 50; Flow rate: 1 mL/min; Gradient: 0% B to 0% B; Injection Volume: 5 ul mL) to afford Isomer-1 (27.30 mg; 49.59%) as a white solid, and Isomer-2 (23.80 mg; 43.27%) as a white solid.

### Example 169 (Stereochemistry Assumed)

HPLC 99.93% purity, 2.17 min, MS: m/s=492.10[M+H]⁺. ¹H NMR (300 MHz, DMSO-d6): 8.96 (s, 1H), 8.01 (s, 1H), 7.63 (d, J=8.4 Hz, 2H), 7.30-7.18 (m, 3H), 6.62 (s, 1H), 5.50 (s, 1H), 4.29-4.17 (m, 1H), 4.18 (s, 2H), 3.99 (s, 2H), 3.27 (dd, J=15.0, 7.3 Hz, 3H), 3.09 (s, 3H), 2.82 (s, 2H), 2.46-2.05 (m, 4H), 2.03 (s, 3H).

### Example 170 (Stereochemistry Assumed)

HPLC 99.84% purity, 2.15 min, MS: m/s=492.10[M+H]⁺. ¹H NMR (300 MHz, DMSO-d6): 8.96 (s, 1H), 8.01 (s, 1H), 7.63 (d, J=8.4 Hz, 2H), 7.30-7.18 (m, 3H), 6.62 (s, 1H), 5.50 (s, 1H), 4.29-4.17 (m, 1H), 4.18 (s, 2H), 3.99 (s, 2H), 3.27 (dd, J=15.0, 7.3 Hz, 3H), 3.09 (s, 3H), 2.82 (s, 2H), 2.46-2.05 (m, 4H), 2.03 (s, 3H).

### Example 171: Synthesis of Compound N-[4-(methanesulfonylmethyl)phenyl]-7-{7-methyl-1H-imidazo[4,5-b]pyridin-6-yl}-5H,6H,7H,8H-pyrido[3,4-d]pyrimidin-2-amine

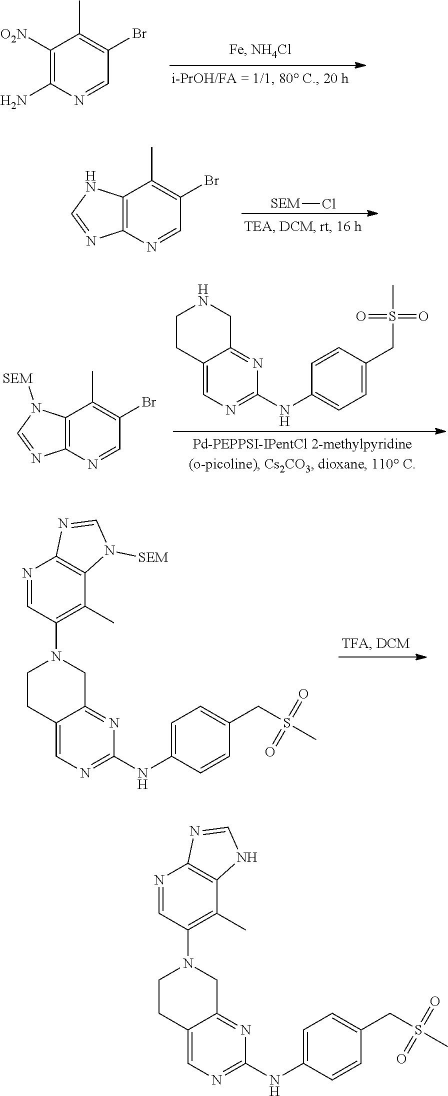

6-bromo-7-methyl-1H-imidazo[4,5-b]pyridine: To a stirred solution of 5-bromo-4-methyl-3-nitropyridin-2-amine (2 g; 8.45 mmol.) in Formic acid (50 ml) and i-PrOH (50 ml) were added NH₄Cl (4.76 g; 84.47 mmol.) and Fe (4.98 g; 84.47 mmol.) in portion at room temperature. After stirring for 2 h at 80 degrees C. The solids were filtered off and the filtrate was concentrated under reduced pressure. The residue was purified by silica gel column chromatography, eluted with CH₂Cl₂/MeOH (10:1) to afford 6-bromo-7-methyl-1H-imidazo[4,5-b]pyridine as a yellow solid (1.10 g; 60.13%).

6-bromo-7-methyl-3-{[2-(trimethylsilyl)ethoxy]methyl}-3H-imidazo[4,5-b]pyridine: To a stirred solution of 6-bromo-7-methyl-1H-imidazo[4,5-b]pyridine (1 g; 4.68 mmol.) and [2-(chloromethoxy)ethyl]trimethylsilane (867.75 mg; 5.15 mmol.) in THF (20.00 ml) was added NaH (18.70 mg; 0.47 mmol.) in portion at 0 degrees C. After stirring for 16 h at room temperature. The reaction was quenched by ice/water and extracted by EtOAc. The organic layers were combined, dried with anhydrous Na₂SO₄ and concentrated under vacuum. The residue was purified by silica gel column chromatography, eluted with PE/EA (1:1) to afford 6-bromo-7-methyl-3-{[2-(trimethylsilyl)ethoxy]methyl}-3H-imidazo[4,5-b]pyridine as a colorless oil (920 mg; 56.04%).

N-[4-(methanesulfonylmethyl)phenyl]-7-(7-methyl-1-{[2-(trimethylsilyl)ethoxy]methyl}-1H-imidazo[4,5-b]pyridin-6-yl)-5H,6H,7H,8H-pyrido[3,4-d]pyrimidin-2-amine: To a stirred solution of 6-bromo-7-methyl-1-{[2-(trimethylsilyl)ethoxy]methyl}-1H-imidazo[4,5-b]pyridine (313.99 mg; 0.90 mmol.) and N-[4-(methanesulfonylmethyl)phenyl]-5H,6H,7H,8H-pyrido[3,4-d]pyrimidin-2-amine (300 mg; 0.90 mmol.) in Dioxane-1,4 (12.00 ml) was added Pd-PEPPSI-IPentCl 2-methylpyridine (o-picoline) (39.62 mg; 0.04 mmol.) and Cs₂CO₃ (614.57 mg; 1.79 mmol.) at room temperature. The resulting mixture was stirred for 3 h at 105 degrees C. under nitrogen atmosphere. The resulting mixture was concentrated under reduced pressure. The residue was purified by silica gel column chromatography, eluted with PE/EA (1:10) to afford N-[4-(methanesulfonylmethyl)phenyl]-7-(7-methyl-1-{[2-(trimethylsilyl)ethoxy]methyl}-1H-imidazo[4,5-b]pyridin-6-yl)-5H,6H,7H,8H-pyrido[3,4-d]pyrimidin-2-amine as a yellow solid (260 mg; 48.84%).

N-[4-(methanesulfonylmethyl)phenyl]-7-{7-methyl-1H-imidazo[4,5-b]pyridin-6-yl}-5H,6H,7H,8H-pyrido[3,4-d]pyrimidin-2-amine: To a stirred solution of N-[4-(methanesulfonylmethyl)phenyl]-7-(7-methyl-1-{[2-(trimethylsilyl)ethoxy]methyl}-1H-imidazo[4,5-b]pyridin-6-yl)-5H,6H,7H,8H-pyrido[3,4-d]pyrimidin-2-amine (260 mg; 0.44 mmol.) in TFA (3 ml) in portion at 0 degrees C. After stirring for 1 h at 0 degrees C. under nitrogen atmosphere. The resulting mixture was concentrated under reduced pressure. The crude product was purified by Prep-HPLC with following conditions Column: XBridge Prep OBD C18 Column, 30*150 mm, 5 μm; Mobile Phase A: 10 mmol NH₄HCO₃+0.05% NH₄OH, Mobile Phase B: ACN; Flow rate: 60 mL/min; Gradient: 18% B to 48% B in 9 min; Wavelength: 254 nm; RT1 (min): 7; Number Of Runs: 1; to afford N-[4-(methanesulfonylmethyl)phenyl]-7-{7-methyl-1H-imidazo[4,5-b]pyridin-6-yl}-5H,6H,7H,8H-pyrido[3,4-d]pyrimidin-2-amine as a white solid (22.90 mg; 11.25%).

HPLC: 96.63%, 2.48 min; M/S: m/z=450.05. ¹H NMR (300 MHz, DMSO-d6): 12.74 (s, 1H), 9.64 (s, 1H), 8.39 (s, 1H), 8.31 (s, 1H), 8.24 (s, 1H), 7.79 (d, J=8.5 Hz, 2H), 7.29 (d, J=8.5 Hz, 2H), 4.37 (s, 2H), 4.13 (s, 2H), 3.34 (bs, 5H), 2.87 (bs, 5H).

### Example 172: Synthesis of Compound N-{4-[(2-methoxyethanesulfonyl)methyl]phenyl}-6-{8-methyl-1H,2H,3H-pyrido[2,3-b][1,4]oxazin-7-yl}-5,6,7,8-tetrahydro-2,6-naphthyridin-3-amine

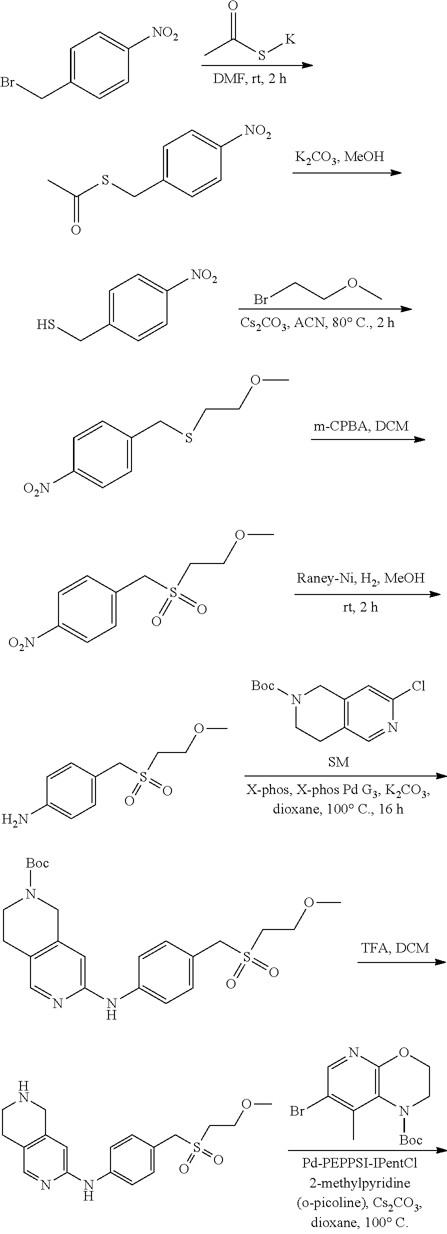

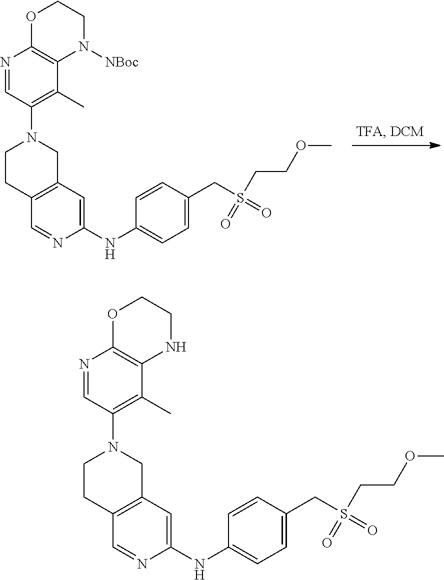

1-{[(4-nitrophenyl)methyl]sulfanyl}ethan-1-one: To a stirred solution of 1-(bromomethyl)-4-nitrobenzene (10 g; 43.97 mmol.) in DMF (100 ml) was added 1-(potassiosulfanyl)ethan-1-one (6.34 g; 52.77 mmol.) in portion at room temperature. After stirring for 16 h at 25 degrees C. The reaction was quenched by ice/water and extracted with EtOAc. The combined organic layers were washed with NaCl(aq.), dried over anhydrous Na₂SO₄. After filtration, the filtrate was concentrated under reduced pressure to afford 1-{[(4-nitrophenyl)methyl]sulfanyl}ethan-1-one as a black solid (9.1 g; 97.65%; Crude Product).

(4-nitrophenyl)methanethiol: To a stirred solution of 1-{[(4-nitrophenyl)methyl]sulfanyl}ethan-1-one (9.1 g; 45.98 mmol.) in MeOH (40 ml) was added acetyl chloride (4 ml) in portion at room temperature. After stirring for 16 h at 30 degrees C. The resulting mixture was concentrated under reduced pressure. The residue was purified by silica gel column chromatography, eluted with PE/EA (10:1) to afford (4-nitrophenyl)methanethiol as a yellow solid (6.50 g; 82.98%)

1-{[(2-methoxyethyl)sulfanyl]methyl}-4-nitrobenzene: To a stirred solution of (4-nitrophenyl)methanethiol (1.1 g; 6.46 mmol.) and 1-bromo-2-methoxyethane (1.13 g; 7.75 mmol.) in ACN (10 ml) was added Cs₂CO₃ (4.43 g; 12.91 mmol.) in portion at room temperature. The resulting mixture was stirred for 3 h at 80 degrees C. under nitrogen atmosphere. The resulting mixture was concentrated under reduced pressure. The residue was purified by silica gel column chromatography, eluted with PE/EA (10:1) to afford 1-{[(2-methoxyethyl)sulfanyl]methyl}-4-nitrobenzene as a yellow oil (780 mg; 52.92%).

1-[(2-methoxyethanesulfonyl)methyl]-4-nitrobenzene: To a stirred solution of 1-{[(2-methoxyethyl)sulfanyl]methyl}-4-nitrobenzene (770 mg; 3.37 mmol) in DCM (12 ml) was added m-CPBA (2451 mg; 13.49 mmol.) at 0 degrees C. The resulting mixture was stirred for 16 h at 25 degrees C. under nitrogen atmosphere. The resulting mixture was concentrated under reduced pressure. The residue was purified by silica gel column chromatography, eluted with PE/EA (1:1) to afford 1-[(2-methoxyethanesulfonyl)methyl]-4-nitrobenzene as a white solid (750 mg; 84.71%).

4-[(2-methoxyethanesulfonyl)methyl]aniline: To a solution of 1-[(2-methoxyethanesulfonyl)methyl]-4-nitrobenzene (700 mg; 2.67 mmol.) in MeOH (20 ml) was added Raney-Ni (276.63 mg; 3.07 mmol.) at room temperature. After stirring for 1 h at room temperature under hydrogen atmosphere. The solids were filtered off and the filtered cake was washed with MeOH and then filtrate was concentrated under reduced pressure to afford 4-[(2-methoxyethanesulfonyl)methyl]aniline as a white solid (550 mg; 87.77%; Crude Product).

tert-butyl 7-({4-[(2-methoxyethanesulfonyl)methyl]phenyl}amino)-1,2,3,4-tetrahydro-2,6-naphthyridine-2-carboxylate: To a stirred solution of tert-butyl 7-chloro-1,2,3,4-tetrahydro-2,6-naphthyridine-2-carboxylate (300 mg; 1.09 mmol) and 4-[(2-methoxyethanesulfonyl)methyl]aniline (307.26 mg; 1.31 mmol) in Dioxane-1,4 (8.00 ml) was added X-PHOS (109.83 mg; 0.22 mmol), Xphos Pd G3 (97.51 mg; 0.11 mmol) and K₂CO₃ (317.73 mg; 2.19 mmol) in portion at room temperature. The resulting mixture was stirred for 3 h at 100 degrees C. under nitrogen atmosphere. The resulting mixture was concentrated under reduced pressure. The residue was purified by silica gel column chromatography, eluted with PE/EA (1:1) to afford tert-butyl 7-({4-[(2-methoxyethanesulfonyl)methyl]phenyl}amino)-1,2,3,4-tetrahydro-2,6-naphthyridine-2-carboxylate as a yellow solid (390 mg; 76.40%).

N-{4-[(2-methoxyethanesulfonyl)methyl]phenyl}-5,6,7,8-tetrahydro-2,6-naphthyridin-3-amine: To a stirred solution of tert-butyl 7-({4-[(2-methoxyethanesulfonyl)methyl]phenyl}amino)-1,2,3,4-tetrahydro-2,6-naphthyridine-2-carboxylate (380 mg; 0.81 mmol.) in DCM (5 ml) was added TFA (2 ml) in portion at room temperature. After stirring for 1 h at room temperature. The mixture was basified to pH [7] with saturated NaHCO₃ (aq.). The resulting mixture was extracted with EtOAc. The combined organic layers were washed with NaCl(aq.), dried over anhydrous Na₂SO₄. After filtration, the filtrate was concentrated under reduced pressure to afford N-{4-[(2-methoxyethanesulfonyl)methyl]phenyl}-5,6,7,8-tetrahy dro-2,6-naphthyridin-3-amine as a yellow solid (310 mg; 91.55%; Crude Product).

tert-butyl 7-[7-({4-[(2-methoxyethanesulfonyl)methyl]phenyl}amino)-1,2,3,4-tetrahydro-2,6-naphthyridin-2-yl]-8-methyl-1H,2H,3H-pyrido[2,3-b][1,4]oxazine-1-carboxylate: To a stirred solution of N-{4-[(2-methoxyethanesulfonyl)methyl]phenyl}-5,6,7,8-tetrahy dro-2,6-naphthyridin-3-amine (300 mg; 0.72 mmol.) and tert-butyl 7-bromo-8-methyl-1H,2H,3H-pyrido[2,3-b][1,4]oxazine-1-carboxylate (250 mg; 0.72 mmol.) in Dioxane-1,4 (8 ml) was added Pd-PEPPSI-IPentCl 2-methylpyridine (o-picoline) (31.93 mg; 0.04 mmol.) and Cs₂CO₃ (495.35 mg; 1.44 mmol.) at room temperature. The resulting mixture was stirred for 3 h at 100 degrees C. under nitrogen atmosphere. The resulting mixture was concentrated under reduced pressure. The residue was purified by silica gel column chromatography, eluted with DCM/MeOH (10:1) to afford tert-butyl 7-[7-({4-[(2-methoxyethanesulfonyl)methyl]phenyl}amino)-1,2,3,4-tetrahydro-2,6-naphthyridin-2-yl]-8-methyl-1H,2H,3H-pyrido[2,3-b][1,4]oxazine-1-carboxylate as a yellow solid (260 mg; 50.20%).

N-{4-[(2-methoxyethanesulfonyl)methyl]phenyl}-6-{8-methyl-1H,2H,3H-pyrido[2,3-b][1,4]oxazin-7-yl}-5,6,7,8-tetrahydro-2,6-naphthyridin-3-amine: To a stirred solution of tert-butyl 7-[7-({4-[(2-methoxyethanesulfonyl)methyl]phenyl}amino)-1,2,3,4-tetrahydro-2,6-naphthyridin-2-yl]-8-methyl-1H,2H,3H-pyrido[2,3-b][1,4]oxazine-1-carboxylate (250 mg; 0.35 mmol.) in DCM (10 ml) was added TFA (2 ml) in portion at room temperature. The resulting mixture was stirred for 1 h at 25 degrees C. under nitrogen atmosphere. The mixture was basified to pH [7] with saturated NaHCO₃ (aq.) The resulting mixture was extracted with EtOAc. The combined organic layers were washed with NaCl(aq.), dried over anhydrous Na₂SO₄. After filtration, the filtrate was concentrated under reduced pressure. The residue was purified by Prep-HPLC with following conditions (Column: XBridge Shield RP18 OBD Column, 30*150 mm, 5 μm; Mobile Phase A: Water (10 mmol/L NH₄HCO₃+0.05% NH₄OH), Mobile Phase B: ACN; Flow rate: 60 mL/min; Gradient: 20% B to 50% B in 9 min; Wavelength: 254 nm; RT1 (min): 7; Number Of Runs: 3) to afford N-{4-[(2-methoxyethanesulfonyl)methyl]phenyl}-6-{8-methyl-1H,2H,3H-pyrido[2,3-b][1,4]oxazin-7-yl}-5,6,7,8-tetrahydro-2,6-naphthyridin-3-amine as a white solid (97.10 mg; 53.63%).

HPLC: 98.11%, 3.84 min; M/S: m/z=510.15. ¹H NMR (300 MHz, DMSO-d6): 8.99 (s, 1H), 8.02 (s, 1H), 7.63 (d, J=8.6 Hz, 2H), 7.30-7.20 (m, 3H), 6.63 (s, 1H), 5.52 (s, 1H), 4.34 (s, 2H), 4.19 (t, J=4.4 Hz, 2H), 4.00 (s, 2H), 3.71 (t, J=5.8 Hz, 2H), 3.32-3.24 (m, 7H), 3.09 (d, J=6.1 Hz, 2H), 2.84 (d, J=5.6 Hz, 2H), 2.06 (s, 3H).

### Example 173 synthesis of compound 1-(3-hydroxy-3-methylazetidin-1-yl)-2-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}ethan-1-one

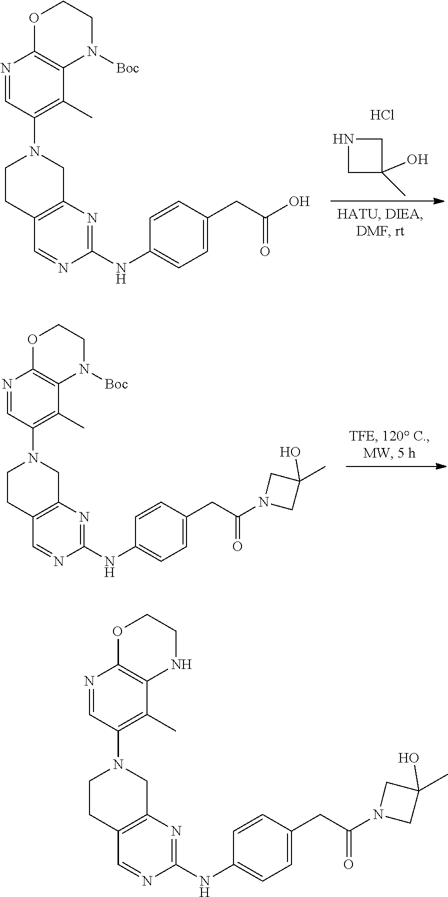

tert-butyl 7-[2-({4[-2-(3-hydroxy-3-methylazetidin-1-yl)-2-oxoethyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate: To a stirred solution of 2-{4-[(7-{1-[(tert-butoxy)carbonyl]-8-methyl-1H,2H,3H-pyrido[2,3-b][1,4]oxazin-7-yl}-5H,6H,7H,8H-pyrido[3,4-d]pyrimidin-2-yl)amino]phenyl}acetic acid (200 mg; 0.36 mmol) and 3-methylazetidin-3-ol hydrochloride (93.60 mg; 0.72 mmol) in DMF (8.00 ml) was added HATU (287.97 mg; 0.72 mmol) and DIEA (146.78 mg; 1.08 mmol) in portion at room temperature. The resulting mixture was stirred for 2 h at room temperature under nitrogen atmosphere. The resulting mixture was concentrated under reduced pressure. The residue was purified by silica gel column chromatography, eluted with CH₂Cl₂/MeOH (10:1) to afford tert-butyl 7-[2-({4-[2-(3-hydroxy-3-methylazetidin-1-yl)-2-oxoethyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate as a yellow solid (190 mg; 86.80%).

1-(3-hydroxy-3-methylazetidin-1-yl)-2-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}ethan-1-one: A solution of tert-butyl 7-[2-({4-[2-(3-hydroxy-3-methylazetidin-1-yl)-2-oxoethyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate (180 mg; 0.30 mmol.) in TFE (10 ml) was stirred for 5 h at 120 degrees C. under microwave atmosphere. The resulting mixture was concentrated under reduced pressure. The residue was purified by Prep-HPLC with following conditions (Column: XBridge Shield RP18 OBD Column, 30*150 mm, 5 μm; Mobile Phase A: Water (10 mmol/L NH₄HCO₃+0.05% NH₄OH), Mobile Phase B: ACN; Flow rate: 60 mL/min; Gradient: 20% B to 50% B in 9 min, 50% B; Wavelength: 254 nm; RT1 (min): 7; Number Of Runs: 1) to afford 1-(3-hydroxy-3-methylazetidin-1-yl)-2-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}ethan-1-one as a yellow solid (17.70 mg; 11.40%).

HPLC: 95.57%, 3.51 min; M/S: m/z=502.30. ¹H NMR (300 MHz, DMSO-d6): 9.47 (s, 1H), 8.33 (s, 1H), 7.67 (d, J=8.4 Hz, 2H), 7.29 (s, 1H), 7.11 (d, J=8.4 Hz, 2H), 5.63 (s, 1H), 5.53 (s, 1H), 4.20 (s, 2H), 4.01 (d, J=8.5 Hz, 4H), 3.96 (s, 2H), 3.74-3.61 (m, 2H), 3.36 (s, 2H), 3.13 (s, 2H), 2.79 (s, 2H), 2.05 (s, 3H), 1.36 (s, 3H).

### Example 174: Synthesis of Compound 2-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}-1-[3-(propan-2-yloxy)azetidin-1-yl]ethan-1-one

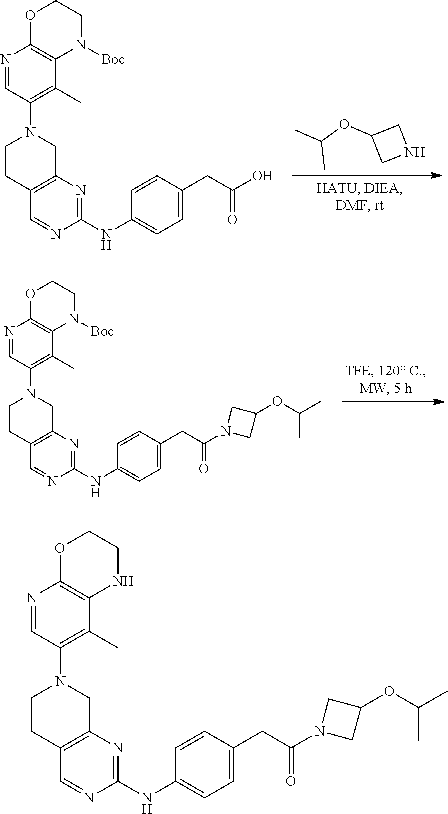

tert-butyl 8-methyl-7-{2-[(4-{2-oxo-2-[3-(propan-2-yloxy)azetidin-1-yl]ethyl}phenyl)amino]-5H,6H,7H,8H-pyrido[3,4-d]pyrimidin-7-yl}-1H,2H,3H-pyrido[2,3-b][1,4]oxazine-1-carboxylate: To a stirred solution of 2-{4-[(7-{1-[(tert-butoxy)carbonyl]-8-methyl-1H,2H,3H-pyrido[2,3-b][1,4]oxazin-7-yl}-5H,6H,7H,8H-pyrido[3,4-d]pyrimidin-2-yl)amino]phenyl}acetic acid (200 mg; 0.36 mmol) and 3-(propan-2-yloxy)azetidine hydrochloride (114.84 mg; 0.72 mmol) in DMF (5.00 ml) was added HATU (287.97 mg; 0.72 mmol) and DIEA (146.78 mg; 1.08 mmol) in portion at room temperature. The resulting mixture was stirred for 2 h at room temperature under nitrogen atmosphere. The resulting mixture was concentrated under reduced pressure. The residue was purified by silica gel column chromatography, eluted with CH₂Cl₂/MeOH (10:1) to afford tert-butyl 8-methyl-7-{2-[(4-{2-oxo-2-[3-(propan-2-yloxy)azetidin-1-yl]ethyl}phenyl)amino]-5H,6H,7H,8H-pyrido[3,4-d]pyrimidin-7-yl}-1H,2H,3H-pyrido[2,3-b][1,4]oxazine-1-carboxylate as a yellow solid (180 mg; 76.97%).

2-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}-1-[3-(propan-2-yloxy)azetidin-1-yl]ethan-1-one: A solution of tert-butyl 8-methyl-7-{2-[(4-{2-oxo-2-[3-(propan-2-yloxy)azetidin-1-yl]ethyl}phenyl)amino]-5H,6H,7H,8H-pyrido[3,4-d]pyrimidin-7-yl}-1H,2H,3H-pyrido[2,3-b][1,4]oxazine-1-carboxylate (170 mg; 0.26 mmol.) in TFE (10 ml) was stirred for 5 h at 120 degrees C. under microwave atmosphere. The resulting mixture was concentrated under reduced pressure. The residue was purified by Prep-HPLC with following conditions Column: XBridge Prep Phenyl OBD Column, 19*100 mm, 5 μm; Mobile Phase A: Water (10 mmol/L NH₄HCO₃+0.05% NH₄OH), Mobile Phase B: ACN; Flow rate: 60 mL/min; Gradient: 30% B to 60% B in 8 min; Wavelength: 254 nm; RT1 (min): 7; Number Of Runs: 2) to afford 2-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}-1-[3-(propan-2-yloxy)azetidin-1-yl]ethan-1-one as a brown solid (59.00 mg; 42.24%).

HPLC: 99.17%, 4.37 min; M/S: m/z=530.35. ¹H NMR (300 MHz, DMSO-d6): 9.47 (s, 1H), 8.33 (s, 1H), 7.71-7.62 (m, 2H), 7.29 (s, 1H), 7.11 (d, J=8.5 Hz, 2H), 5.53 (s, 1H), 4.40-4.31 (m, 2H), 4.24-4.15 (m, 2H), 4.05 (dd, J=11.0, 5.7 Hz, 1H), 3.94 (d, J=9.7 Hz, 3H), 3.66-3.50 (m, 2H), 3.24 (s, 2H), 3.12 (s, 2H), 3.06 (s, 2H), 2.79 (s, 2H), 2.05 (s, 3H), 1.08 (d, J=6.1 Hz, 6H).

### Example 175 synthesis of compound 1-[3-(methoxymethyl)azetidin-1-yl]-2-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}ethan-1-one

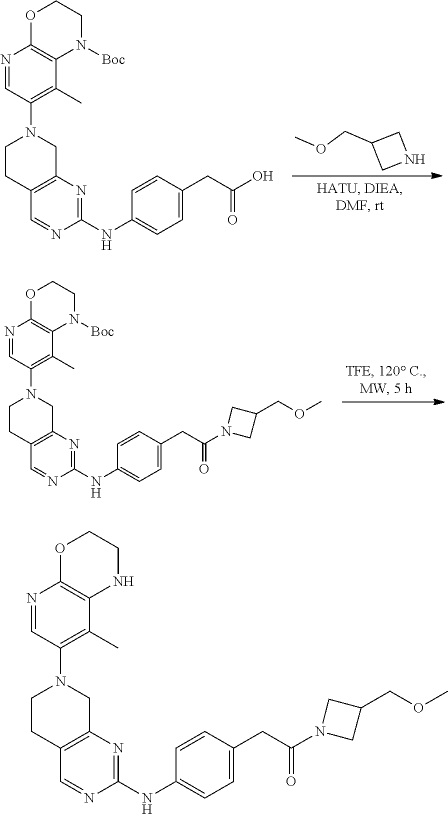

tert-butyl 7-{2-[(4-{2-[3-(methoxymethyl)azetidin-1-yl]-2-oxoethyl}phenyl)amino]-5H,6H,7H,8H-pyrido[3,4-d]pyrimidin-7-yl}-8-methyl-1H,2H,3H-pyrido[2,3-b][1,4]oxazine-1-carboxylate: To a stirred solution of 2-{4-[(7-{1-[(tert-butoxy)carbonyl]-8-methyl-1H,2H,3H-pyrido[2,3-b][1,4]oxazin-7-yl}-5H,6H,7H,8H-pyrido[3,4-d]pyrimidin-2-yl)amino]phenyl}acetic acid (200 mg; 0.36 mmol.) and 3-(methoxymethyl)azetidine hydrochloride (104.22 mg; 0.72 mmol.) in DMF (8 ml) was added HATU (287.97 mg; 0.72 mmol.) and DIEA (146.78 mg; 1.08 mmol.) in portion at room temperature. The resulting mixture was stirred for 16 h at room temperature under nitrogen atmosphere. The resulting mixture was concentrated under reduced pressure. The residue was purified by silica gel column chromatography, eluted with CH₂Cl₂/MeOH (10:1) to afford tert-butyl 7-{2-[(4-{2-[3-(methoxymethyl)azetidin-1-yl]-2-oxoethyl}phenyl)amino]-5H,6H,7H,8H-pyrido[3,4-d]pyrimidin-7-yl}-8-methyl-1H,2H,3H-pyrido[2,3-b][1,4]oxazine-1-carboxylate as a yellow solid (190.00 mg; 82.00%).

1-[3-(methoxymethyl)azetidin-1-yl]-2-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}ethan-1-one: A solution of 7-{2-[(4-{2-[3-(methoxymethyl)azetidin-1-yl]-2-oxoethyl}phenyl)amino]-5H,6H,7H,8H-pyrido[3,4-d]pyrimidin-7-yl}-8-methyl-1H,2H,3H-pyrido[2,3-b][1,4]oxazine-1-carboxylate (190 mg; 0.30 mmol.) in TFE (10 ml) was stirred for 5 h under MW at 120 degrees C. The resulting mixture was concentrated under reduced pressure. The residue was purified by Prep-HPLC with following conditions Column: XBridge Prep Phenyl OBD Column, 19*100 mm, 5 μm; Mobile Phase A: Water (10 mmol/L NH₄HCO₃+0.05% NH₄OH), Mobile Phase B: ACN; Flow rate: 60 mL/min; Gradient: 30% B to 60% B in 8 min, 60% B; Wavelength: 254 nm; RT1 (min): 7; Number Of Runs: 4) to afford 1-[3-(methoxymethyl)azetidin-1-yl]-2-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}ethan-1-one as a brown solid (77.7 mg; 50.59%).

HPLC: 99.03%,3.91 min; M/S: m/z=516.25. ¹H NMR (300 MHz, DMSO-d6): 9.47 (s, 1H), 8.32 (s, 1H), 7.72-7.62 (m, 2H), 7.29 (s, 1H), 7.10 (d, J=8.5 Hz, 2H), 5.53 (s, 1H), 4.24-4.12 (m, 3H), 3.95 (s, 2H), 3.92-3.81 (m, 2H), 3.54 (dd, J=9.6, 5.6 Hz, 1H), 3.45 (d, J=6.5 Hz, 2H), 3.42 (s, 4H), 3.30 (s, 3H), 3.12 (t, J=5.5 Hz, 2H), 2.78 (q, J=7.0, 6.3 Hz, 3H), 2.04 (s, 3H).

### Example 176: Synthesis of Compound N-{5-[(cyclopropanesulfonyl)methyl]pyridin-2-yl}-6-{8-methyl-1H,2H,3H-pyrido[2,3-b][1,4]oxazin-7-yl}-5,6,7,8-tetrahydro-2,6-naphthyridin-3-amine

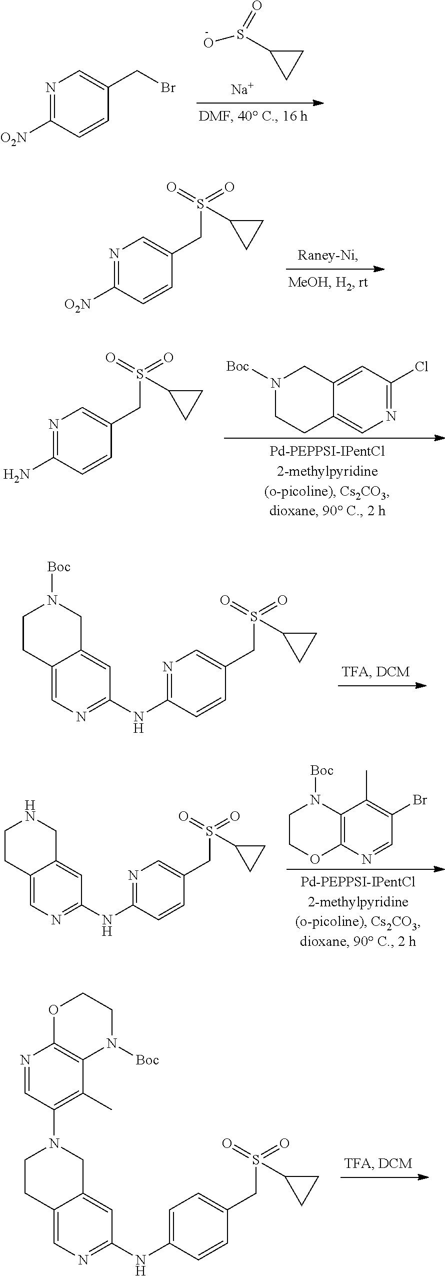

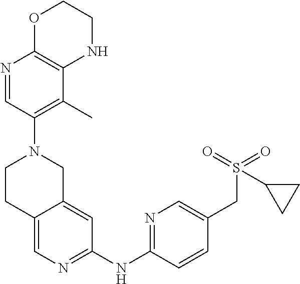 [SMILES: Cc1c(N2CCc3cnc(Nc4ccc(CS(=O)(=O)C5CC5)cn4)cc3C2)cnc2c1NCCO2]

5-[(cyclopropanesulfonyl)methyl]-2-nitropyridine: To a stirred solution of 5-(bromomethyl)-2-nitropyridine (1 g; 4.48 mmol) in DMF (10 ml) was added sodium cyclopropanesulfinate (1.21 g; 8.95 mmol) in portion at room temperature. The resulting mixture was stirred for 2 h at 65 degrees C. under nitrogen atmosphere. The resulting mixture was concentrated under reduced pressure. The residue was purified by silica gel column chromatography, eluted with PE/EA (1:100) to afford 5-[(cyclopropanesulfonyl)methyl]-2-nitropyridine as a white solid (980 mg; 89.40%).

5-[(cyclopropanesulfonyl)methyl]pyridin-2-amine: To a solution of 5-[(cyclopropanesulfonyl)methyl]-2-nitropyridine (980 mg; 4.00 mmol) in MeOH (20 ml) was added Raney-Ni (415.02 mg; 4.60 mmol.) at room temperature. After stirring for 1 h at room temperature under hydrogen atmosphere. The resulting mixture was filtered, the filter cake was washed with MeOH and then filtrate was concentrated under reduced pressure to afford 5-[(cyclopropanesulfonyl)methyl]pyridin-2-amine as a purple solid (710 mg; 82.45%; Crude Product).

tert-butyl 7-({5-[(cyclopropanesulfonyl)methyl]pyridin-2-yl}amino)-1,2,3,4-tetrahydro-2,6-naphthyridine-2-carboxylate: To a stirred solution of tert-butyl 7-chloro-1,2,3,4-tetrahydro-2,6-naphthyridine-2-carboxylate (300 mg; 1.09 mmol.) and 5-[(cyclopropanesulfonyl)methyl]pyridin-2-amine (282.51 mg; 1.31 mmol.) in Dioxane-1,4 (10.00 ml) was added Pd-PEPPSI-IPentCl 2-methylpyridine (o-picoline) (96.75 mg; 0.11 mmol.) and Cs₂CO₃ (750.41 mg; 2.19 mmol.) in portion at room temperature. The resulting mixture was stirred for 3 h at 100 degrees C. under nitrogen atmosphere. The resulting mixture was concentrated under reduced pressure. The residue was purified by silica gel column chromatography, eluted with CH₂Cl₂/MeOH (10:1) to afford tert-butyl 7-({5-[(cyclopropanesulfonyl)methyl]pyridin-2-yl}amino)-1,2,3,4-tetrahydro-2,6-naphthyridine-2-carboxylate as a white solid (400 mg; 80.61%).

N-{5-[(cyclopropanesulfonyl)methyl]pyridin-2-yl}-5,6,7,8-tetrahydro-2,6-naphthyridin-3-amine: To a stirred solution of tert-butyl 7-({5-[(cyclopropanesulfonyl)methyl]pyridin-2-yl}amino)-1,2,3,4-tetrahydro-2,6-naphthyridine-2-carboxylate (400 mg; 0.88 mmol.) in DCM (10 ml) was added TFA (2 ml) in portion at room temperature. After stirring for 1 h at room temperature. The mixture was basified to pH [7] with saturated NaHCO₃ (aq.). The resulting mixture was extracted with EtOAc. The combined organic layers were washed with NaCl(aq.), dried over anhydrous Na₂SO₄. After filtration, the filtrate was concentrated under reduced pressure to afford N-{5-[(cyclopropanesulfonyl)methyl]pyridin-2-yl}-5,6,7,8-tetrahy dro-2,6-naphthyridin-3-amine as a yellow solid (300 mg; 97.84%; Crude Product).

tert-butyl 7-[7-({5-[(cyclopropanesulfonyl)methyl]pyridin-2-yl}amino)-1,2,3,4-tetrahydro-2,6-naphthyridin-2-yl]-8-methyl-1H,2H,3H-pyrido[2,3-b][1,4]oxazine-1-carboxylate: To a stirred solution of N-{5-[(cyclopropanesulfonyl)methyl]pyridin-2-yl}-5,6,7,8-tetrahydro-2,6-naphthyridin-3-amine (297.47 mg; 0.75 mmol) and tert-butyl 7-bromo-8-methyl-1H,2H,3H-pyrido[2,3-b][1,4]oxazine-1-carboxylate (260 mg; 0.75 mmol) in Dioxane-1,4 (8 ml) was added Pd-PEPPSI-IPentCl 2-methylpyridine (o-picoline) (33.21 mg; 0.04 mmol) and Cs₂CO₃ (515.16 mg; 1.50 mmol) at room temperature. The resulting mixture was stirred for 3 h at 100 degrees C. under nitrogen atmosphere. The resulting mixture was concentrated under reduced pressure. The residue was purified by silica gel column chromatography, eluted with DCM/MeOH (10:1) to afford tert-butyl 7-[7-({5-[(cyclopropanesulfonyl)methyl]pyridin-2-yl}amino)-1,2,3,4-tetrahydro-2,6-naphthyridin-2-yl]-8-methyl-1H,2H,3H-pyrido[2,3-b][1,4]oxazine-1-carboxylate as a yellow solid (240 mg; 51.42%).

N-{5-[(cyclopropanesulfonyl)methyl]pyridin-2-yl}-6-{8-methyl-1H,2H,3H-pyrido[2,3-b][1,4]oxazin-7-yl}-5,6,7,8-tetrahydro-2,6-naphthyridin-3-amine: To a stirred solution of tert-butyl 7-[7-({5-[(cyclopropanesulfonyl)methyl]pyridin-2-yl}amino)-1,2,3,4-tetrahydro-2,6-naphthyridin-2-yl]-8-methyl-1H,2H,3H-pyrido[2,3-b][1,4]oxazine-1-carboxylate (240 mg; 0.36 mmol) in DCM (10 ml) was added TFA (2 ml) in portion at room temperature. The resulting mixture was stirred for 1 h at 25 degrees C. under nitrogen atmosphere. The filtrate was concentrated under reduced pressure. The residue was purified by Prep-HPLC with following conditions (Column: XBridge Prep OBD C18 Column, 30*150 mm, 5 μm; Mobile Phase A: Water (10 mmol/L NH₄HCO₃+0.05% NH₄OH), Mobile Phase B: ACN; Flow rate: 60 mL/min; Gradient: 20% B to 50% B in 9 min, Wavelength: 254 nm; RT1 (min): 7; Number Of Runs: 4) to afford N-{5-[(cyclopropanesulfonyl)methyl]pyridin-2-yl}-6-{8-methyl-1H,2H,3H-pyrido[2,3-b][1,4]oxazin-7-yl}-5,6,7,8-tetrahydro-2,6-naphthyridin-3-amine as a white solid (91.6 mg; 51.63%)

HPLC: 99.56%, 3.35 min; M/S: m/z=493.10. ¹H NMR (400 MHz, DMSO-d6): 9.72 (s, 1H), 8.20 (d, J=2.2 Hz, 1H), 8.09 (s, 1H), 7.66 (dd, J=8.7, 2.4 Hz, 1H), 7.60 (d, J=8.6 Hz, 1H), 7.56 (s, 1H), 7.28 (s, 1H), 5.52 (s, 1H), 4.46 (s, 2H), 4.19 (t, J=4.4 Hz, 2H), 4.05 (s, 2H), 3.25 (s, 2H), 3.10 (t, J=5.7 Hz, 2H), 2.86 (t, J=5.8 Hz, 2H), 2.64-2.52 (m, 1H), 2.04 (s, 3H), 0.98 (dt, J=7.1, 3.3 Hz, 2H), 0.95-0.84 (m, 2H)

### Example 177 synthesis of compound 7-{8-chloro-1H,2H,3H-pyrido[2,3-b][1,4]oxazin-7-yl}-N-[4-(methanesulfonylmethyl)-3-methylphenyl]-5H,6H,7H,8H-pyrido[3,4-d]pyrimidin-2-amine

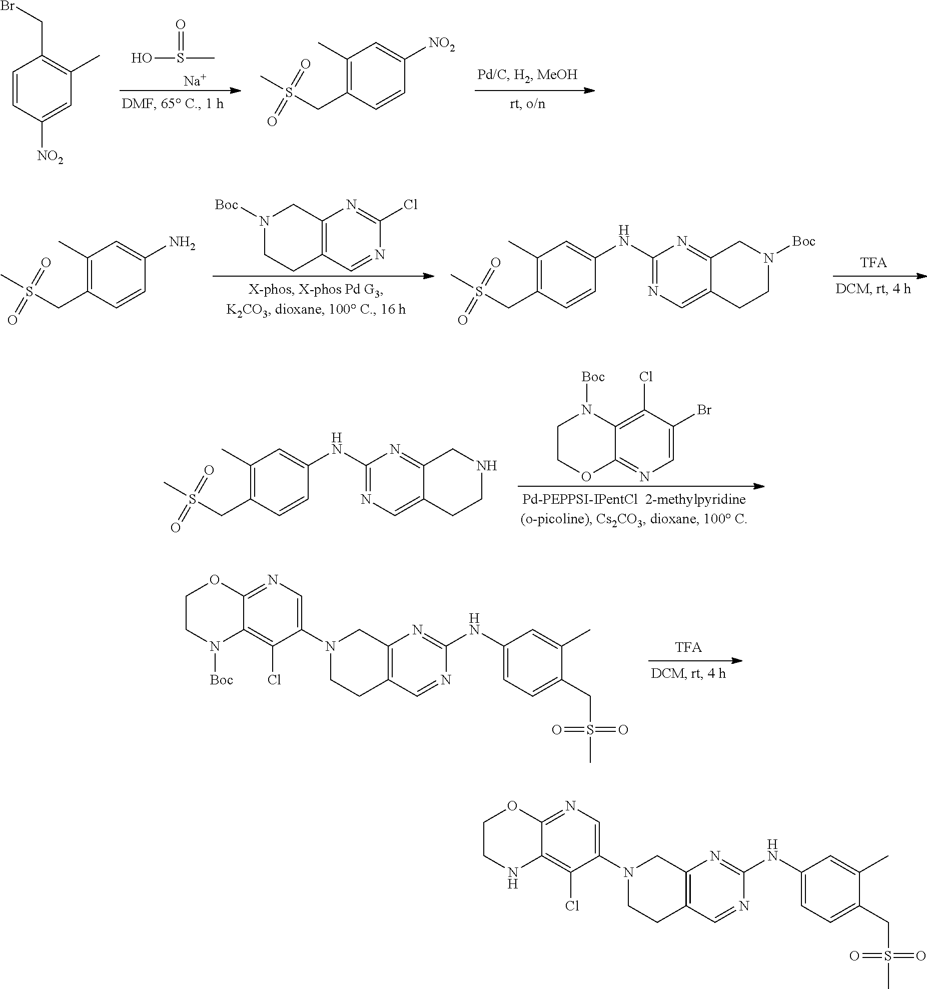

1-(methanesulfonylmethyl)-2-methyl-4-nitrobenzene: To a stirred mixture of 1-(bromomethyl)-2-methyl-4-nitrobenzene (1 g; 4.13 mmol) in DMF (10 ml) was added sodium methanesulfenic acid (0.45 g; 4.13 mmol;) at room temperature. The resulting mixture was stirred for 3 h at 65 degrees C. under nitrogen atmosphere. The mixture was allowed to cool down to room temperature. The resulting mixture was extracted with EA. The combined organic layers were washed with brine, dried over anhydrous Na₂SO₄. After filtration, the filtrate was concentrated under reduced pressure. The residue was purified by silica gel column chromatography, eluted with PE:EA (1:2) to afford 1-(methanesulfonylmethyl)-2-methyl-4-nitrobenzene as a off-white solid (860 mg; 90.7%).

4-(methanesulfonylmethyl)-3-methylaniline: To a stirred mixture of 1-(methanesulfonylmethyl)-2-methyl-4-nitrobenzene (820 mg; 3.57 mmol) in MeOH (10 ml) was added Pd/C (820 mg; 0.77 mmol) at room temperature. The resulting mixture was stirred for overnight at room temperature under hydrogen atmosphere. The resulting mixture was filtered, the filter cake was washed with MeOH and THF. The filtrate was concentrated under reduced pressure to afford 4-(methanesulfonylmethyl)-3-methylaniline as a yellow solid (730 mg; 99.5%).

tert-butyl 2-{[4-(methanesulfonylmethyl)-3-methylphenyl]amino}-5H,6H,7H,8H-pyrido[3,4-d]pyrimidine-7-carboxylate: To a stirred mixture of 4-(methanesulfonylmethyl)-3-methylaniline (640 mg; 3.12 mmol) and tert-butyl 2-chloro-5H,6H,7H,8H-pyrido[3,4-d]pyrimidine-7-carboxylate (1120 mg; 3.74 mmol.) in Dioxane-1,4 (7 ml) were added Xphos Pd G3 (278 mg; 0.31 mmol), 2-dicyclohexyl p-2 minus 2-3 isopropyl biphenyl (156 mg; 0.31 mmol) and K₂CO₃ (906 mg; 6.23 mmol) at room temperature. The resulting mixture was stirred for 1 h at 100 degrees C. under nitrogen atmosphere. The mixture was allowed to cool down to room temperature. The resulting mixture was extracted with EA. The combined organic layers were washed with brine, dried over anhydrous Na₂SO₄. After filtration, the filtrate was concentrated under reduced pressure. The residue was purified by silica gel column chromatography, eluted with PE:EA (1:1) to afford tert-butyl 2-{[4-(methanesulfonylmethyl)-3-methylphenyl]amino}-5H,6H,7H,8H-pyrido[3,4-d]pyrimidine-7-carboxylate as a off-white solid (1.3 g; 94.7%).

N-[4-(methanesulfonylmethyl)-3-methylphenyl]-5H,6H,7H,8H-pyrido[3,4-d]pyrimidin-2-amine: To a stirred mixture of tert-butyl 2-{[4-(methanesulfonylmethyl)-3-methylphenyl]amino}-5H,6H,7H,8H-pyrido[3,4-d]pyrimidine-7-carboxylate (1.2 g; 2.72 mmol) in DCM (12 ml) was added TFA (4 ml) at 0 degrees C. The resulting mixture was stirred for 1 h at room temperature under nitrogen atmosphere. The residue was purified by reverse flash chromatography with the following conditions: column, C18 silica gel; mobile phase, MeOH in water, 40% to 50% gradient in 30 min; detector, UV 254 nm to afford N-[4-(methanesulfonylmethyl)-3-methylphenyl]-5H,6H,7H,8H-pyrido[3,4-d]pyrimidin-2-amine as a yellow solid (900 mg; 97.5%).

tert-butyl 8-chloro-7-(2-{[4-(methanesulfonylmethyl)-3-methylphenyl]amino}-5H,6H,7H,8H-pyrido[3,4-d]pyrimidin-7-yl)-1H,2H,3H-pyrido[2,3-b][1,4]oxazine-1-carboxylate: To a stirred mixture of N-[4-(methanesulfonylmethyl)-3-methylphenyl]-5H,6H,7H,8H-pyrido[3,4-d]pyrimidin-2-amine (200 mg; 0.59 mmol) and tert-butyl 7-bromo-8-chloro-1H,2H,3H-pyrido[2,3-b][1,4]oxazine-1-carboxylate (260 mg; 0.71 mmol) in Dioxane-1,4 (3 ml) were added Pd-PEPPSI-IPentCl 2-methylpyridine (o-picoline) (52 mg; 0.06 mmol) and Cs₂CO₃ (405 mg; 1.18 mmol) at room temperature. The resulting mixture was stirred for 1 h at 100 degrees C. under nitrogen atmosphere. The mixture was allowed to cool down to room temperature. The resulting mixture was extracted with EA. The combined organic layers were washed with brine, dried over anhydrous Na₂SO₄. After filtration, the filtrate was concentrated under reduced pressure. The residue was purified by reverse flash chromatography with the following conditions: column, C18 silica gel; mobile phase, MeOH in water, 45% to 50% gradient in 30 min; detector, UV 254 nm to afford tert-butyl 8-chloro-7-(2-{[4-(methanesulfonylmethyl)-3-methylphenyl]amino}-5H,6H,7H,8H-pyrido[3,4-d]pyrimidin-7-yl)-1H,2H,3H-pyrido[2,3-b][1,4]oxazine-1-carboxylate as a off-white solid (130 mg; 36.5%).

7-{8-chloro-1H,2H,3H-pyrido[2,3-b][1,4]oxazin-7-yl}-N-[4 (methanesulfonylmethyl)-3-methylphenyl]-5H,6H,7H,8H-pyrido[3,4-d]pyrimidin-2-amine: To a stirred mixture of tert-butyl 8-chloro-7-(2-{[4-(methanesulfonylmethyl)-3-methylphenyl]amino}-5H,6H,7H,8H-pyrido[3,4-d]pyrimidin-7-yl)-1H,2H,3H-pyrido[2,3-b][1,4]oxazine-1-carboxylate (100 mg; 0.17 mmol) in DCM (1 ml) was added TFA (0.3 ml) at 0 degrees C. The resulting mixture was stirred for 1.5 h at room temperature under nitrogen atmosphere. The resulting mixture was concentrated under reduced pressure. The crude product was purified by Prep-HPLC with the following conditions (Column: XBridge Prep OBD C18 Column, 30*150 mm; Mobile Phase A: Water (10 mmol/L NH₄HCO₃), Mobile Phase B: ACN; Flow rate: 60 mL/min; Gradient: 23% to 53% in 8 min; Wavelength: 254 nm; RT1 (min): 7; Number Of Runs: 1) to afford 7-{8-chloro-1H,2H,3H-pyrido[2,3-b][1,4]oxazin-7-yl}-N-[4-(methanesulfonylmethyl)-3-methylphenyl]-5H,6H,7H,8H-pyrido[3,4-d]pyrimidin-2-amine as a white solid (12.3 mg; 14.8%).

HPLC: 99.86% purity, 4.60 min. MS: m/z=502 [M+H]⁺. ¹H NMR (300 MHz, DMSO-d6) 9.55 (s, 1H), 8.35 (s, 1H), 7.66 (dd, J=8.4, 2.3 Hz, 1H), 7.58 (d, J=2.3 Hz, 1H), 7.32 (s, 1H), 7.22 (d, J=8.4 Hz, 1H), 6.11 (s, 1H), 4.43 (s, 2H), 4.24 (t, J=4.3 Hz, 2H), 4.08 (s, 2H), 3.35 (s, 2H), 3.26 (t, J=5.7 Hz, 2H), 2.94 (s, 3H), 2.81 (t, J=5.5 Hz, 2H), 2.34 (s, 3H)

### Example 177 and 178: Synthesis of Compound N-[4-(methanesulfonylmethyl)-3-methylphenyl]-7-{8-methyl-1H,2H,3H-pyrido[2,3-b][1,4]oxazin-7-yl}-5H,6H,7H,8H-pyrido[3,4-d]pyrimidin-2-amine

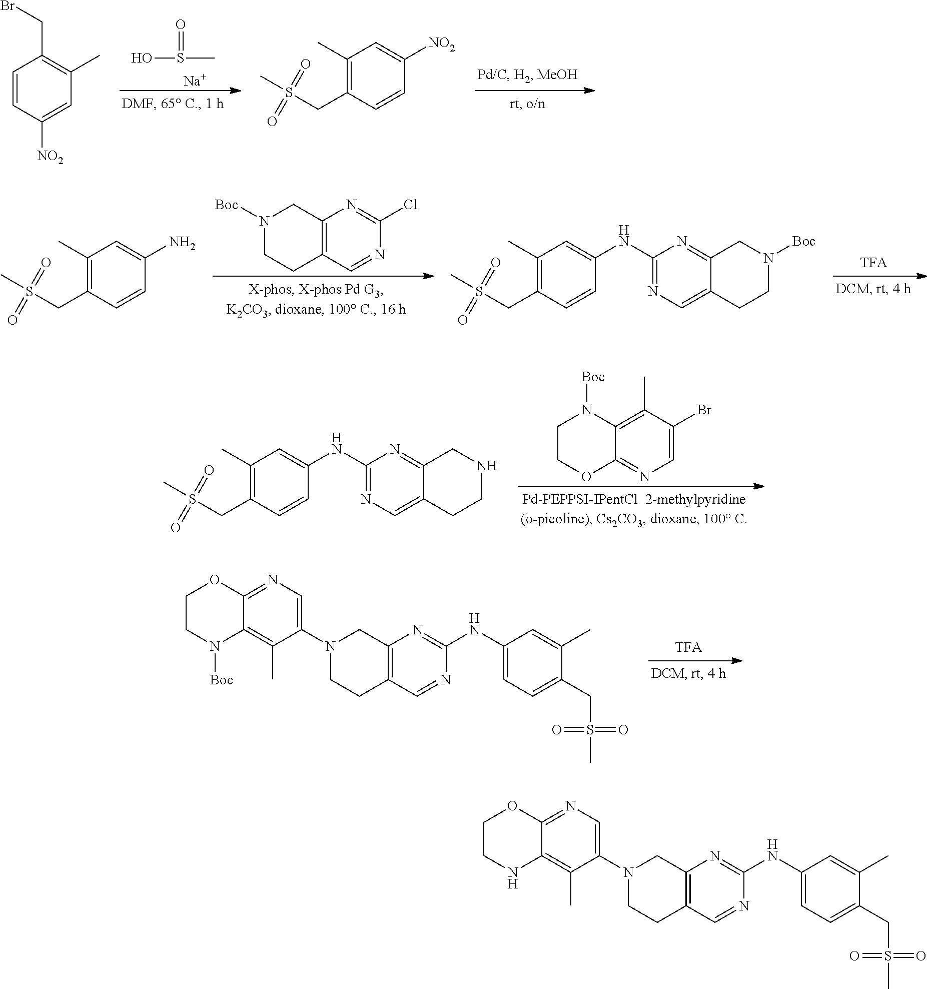

1-(methanesulfonylmethyl)-2-methyl-4-nitrobenzene: To a stirred mixture of 1-(bromomethyl)-2-methyl-4-nitrobenzene (1 g; 4.13 mmol) in DMF (10 ml) was added sodium methanesulfinic acid (0.45 g; 4.13 mmol;) at room temperature. The resulting mixture was stirred for 3 h at 65 degrees C. under nitrogen atmosphere. The mixture was allowed to cool down to room temperature. The resulting mixture was extracted with EA. The combined organic layers were washed with brine, dried over anhydrous Na₂SO₄. After filtration, the filtrate was concentrated under reduced pressure. The residue was purified by silica gel column chromatography, eluted with PE:EA (1:2) to afford 1-(methanesulfonylmethyl)-2-methyl-4-nitrobenzene as a off-white solid (860 mg; 90.7%).

4-(methanesulfonylmethyl)-3-methylaniline: To a stirred mixture of 1-(methanesulfonylmethyl)-2-methyl-4-nitrobenzene (820 mg; 3.57 mmol) in MeOH (10 ml) was added Pd/C (820 mg; 0.77 mmol) at room temperature. The resulting mixture was stirred for overnight at room temperature under hydrogen atmosphere. The resulting mixture was filtered, the filter cake was washed with MeOH and THF. The filtrate was concentrated under reduced pressure to afford 4-(methanesulfonylmethyl)-3-methylaniline as a yellow solid (730 mg; 99.5%).

tert-butyl 2-{[4-(methanesulfonylmethyl)-3-methylphenyl]amino}-5H,6H,7H,8H-pyrido[3,4-d]pyrimidine-7-carboxylate: To a stirred mixture of 4-(methanesulfonylmethyl)-3-methylaniline (640 mg; 3.12 mmol) and tert-butyl 2-chloro-5H,6H,7H,8H-pyrido[3,4-d]pyrimidine-7-carboxylate (1120 mg; 3.74 mmol.) in Dioxane-1,4 (7 ml) were added Xphos Pd G3 (278 mg; 0.31 mmol), 2-dicyclohexyl p−2 minus 2-3 isopropyl biphenyl (156 mg; 0.31 mmol) and K₂CO₃ (906 mg; 6.23 mmol) at room temperature. The resulting mixture was stirred for 1 h at 100 degrees C. under nitrogen atmosphere. The mixture was allowed to cool down to room temperature. The resulting mixture was extracted with EA. The combined organic layers were washed with brine, dried over anhydrous Na₂SO₄. After filtration, the filtrate was concentrated under reduced pressure. The residue was purified by silica gel column chromatography, eluted with PE:EA (1:1) to afford tert-butyl 2-{[4-(methanesulfonylmethyl)-3-methylphenyl]amino}-5H,6H,7H,8H-pyrido[3,4-d]pyrimidine-7-carboxylate as a off-white solid (1.3 g; 94.7%).

N-[4-(methanesulfonylmethyl)-3-methylphenyl]-5H,6H,7H,8H-pyrido[3,4-d]pyrimidin-2-amine: To a stirred mixture of tert-butyl 2-{[4-(methanesulfonylmethyl)-3-methylphenyl]amino}-5H,6H,7H,8H-pyrido[3,4-d]pyrimidine-7-carboxylate (1.2 g; 2.72 mmol) in DCM (12 ml) was added TFA (4 ml) at 0 degrees C. The resulting mixture was stirred for 1 h at room temperature under nitrogen atmosphere. The residue was purified by reverse flash chromatography with the following conditions: column, C18 silica gel; mobile phase, MeOH in water, 40% to 50% gradient in 30 min; detector, UV 254 nm to afford N-[4-(methanesulfonylmethyl)-3-methylphenyl]-5H,6H,7H,8H-pyrido[3,4-d]pyrimidin-2-amine as a yellow solid (900 mg; 97.5%).

tert-butyl 7-(2-{[4-(methanesulfonylmethyl)-3-methylphenyl]amino}-5H,6H,7H,8H-pyrido[3,4-d]pyrimidin-7-yl)-8-methyl-1H,2H,3H-pyrido[2,3-b][1,4]oxazine-1-carboxylate: To a stirred mixture of N-[4-(methanesulfonylmethyl)-3-methylphenyl]-5H,6H,7H,8H-pyrido[3,4-d]pyrimidin-2-amine (200 mg; 0.59 mmol) and tert-butyl 7-bromo-8-methyl-1H,2H,3H-pyrido[2,3-b][1,4]oxazine-1-carboxylate (245 mg; 0.71 mmol) in Dioxane-1,4 (3 ml) were added Pd-PEPPSI-IPentCl 2-methylpyridine (o-picoline) (52 mg; 0.06 mmol) and Cs₂CO₃ (405 mg; 1.18 mmol) at room temperature. The resulting mixture was stirred for 1 h at 100 degrees C. under nitrogen atmosphere. The mixture was allowed to cool down to room temperature. The resulting mixture was extracted with EA. The combined organic layers were washed with brine, dried over anhydrous Na₂SO₄. After filtration, the filtrate was concentrated under reduced pressure. The residue was purified by reverse flash chromatography with the following conditions: column, C18 silica gel; mobile phase, ACN in water, 40% to 50% gradient in 20 min; detector, UV 254 nm to afford tert-butyl 7-(2-{[4-(methanesulfonylmethyl)-3-methylphenyl]amino}-5H,6H,7H,8H-pyrido[3,4-d]pyrimidin-7-yl)-8-methyl-1H,2H,3H-pyrido[2,3-b][1,4]oxazine-1-carboxylate as a yellow solid (130 mg; 37.6%).

N-[4-(methanesulfonylmethyl)-3-methylphenyl]-7-{8-methyl-1H,2H,3H-pyrido[2,3-b][1,4]oxazin-7-yl}-5H,6H,7H,8H-pyrido[3,4-d]pyrimidin-2-amine: To a stirred mixture of tert-butyl 7-(2-{[4-(methanesulfonylmethyl)-3-methylphenyl]amino}-5H,6H,7H,8H-pyrido[3,4-d]pyrimidin-7-yl)-8-methyl-1H,2H,3H-pyrido[2,3-b][1,4]oxazine-1-carboxylate (100 mg; 0.17 mmol) in DCM (1 ml) was added TFA (0.3 ml) at 0 degrees C. The resulting mixture was stirred for 1.5 h at room temperature under nitrogen atmosphere. The resulting mixture was concentrated under reduced pressure. The mixture was basified to pH=9-10 with Saturated sodium bicarbonate aqueous solution. The crude product was purified by Prep-HPLC with the following conditions (Column: XBridge Prep OBD C18 Column, 30*150 mm, 5 μm; Mobile Phase A: 10 mmol NH₄HCO₃+0.05% NH₄OH, Mobile Phase B: ACN; Flow rate: 60 mL/min; Gradient: 23% to 50% in 9 min; Wavelength: 254 nm; RT1 (min): 7; Number Of Runs: 1) to afford N-[4-(methanesulfonylmethyl)-3-methylphenyl]-7-{8-methyl-1H,2H,3H-pyrido[2,3-b][1,4]oxazin-7-yl}-5H,6H,7H,8H-pyrido[3,4-d]pyrimidin-2-amine as a white solid (15.4 mg; 18.2%).

HPLC: 97.02% purity, 3.92 min. MS: m/z=481 [M+H]⁺. ¹H NMR (400 MHz, DMSO-d6) 9.55 (s, 1H), 8.35 (s, 1H), 7.69-7.62 (m, 1H), 7.58 (s, 1H), 7.29 (s, 1H), 7.21 (d, J=8.4 Hz, 1H), 5.52 (s, 1H), 4.42 (s, 2H), 4.19 (s, 2H), 3.97 (s, 2H), 3.12 (s, 4H), 2.94 (s, 3H), 2.80 (s, 2H), 2.33 (s, 3H), 2.04 (s, 3H)

### Example 179: Synthesis of Compound N-[4-(methanesulfonylmethyl)-3-methylphenyl]-6-{8-methyl-1H,2H,3H-pyrido[2,3-b][1,4]oxazin-7-yl}-5,6,7,8-tetrahydro-2,6-naphthyridin-3-amine

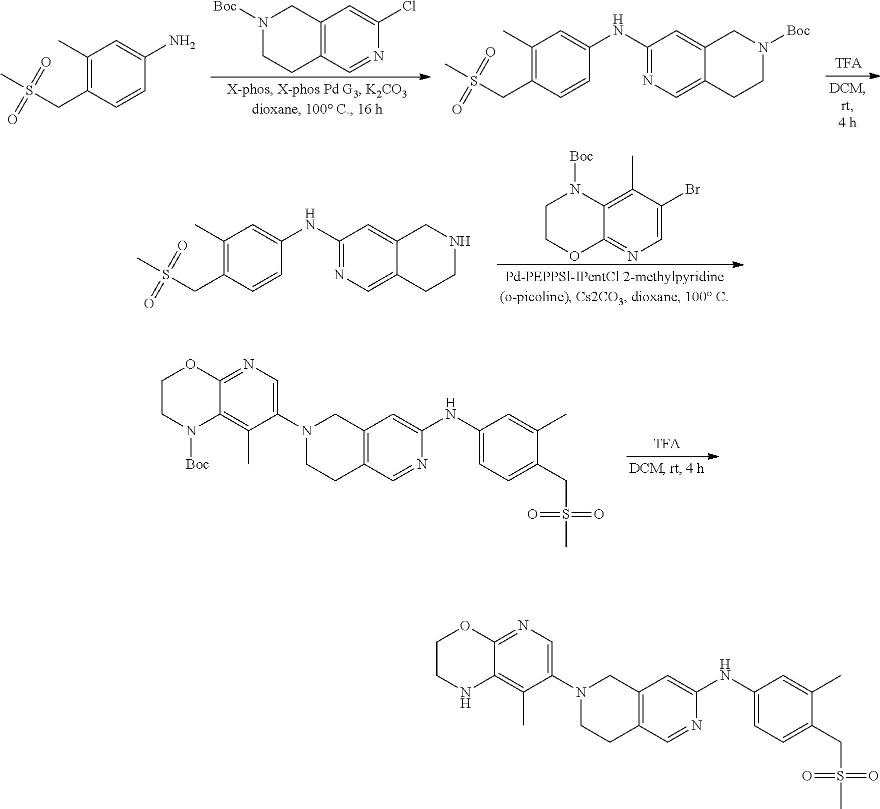

tert-butyl 7-{[4-(methanesulfonylmethyl)-3-methylphenyl]amino}-1,2,3,4-tetrahydro-2,6-naphthyridine-2-carboxylate: To a stirred mixture of 4-(methanesulfonylmethyl)-3-methylaniline (500 mg; 2.41 mmol) and tert-butyl 7-chloro-1,2,3,4-tetrahydro-2,6-naphthyridine-2-carboxylate (793 mg; 2.89 mmol) in Dioxane-1,4 (6 ml) were added Methanesulfonato(2-dicyclohexylphosphino-2′,4′,6′-tri-i-propyl-1,1′-biphenyl)(2′-amino-1,1′-biphenyl-2-yl)palladium(II (215 mg; 0.24 mmol), 2-dicyclohexyl p-2 minus 2-3 isopropyl biphenyl (121 mg; 0.24 mmol) and K₂CO₃ (700 mg; 4.82 mmol) at room temperature. The resulting mixture was stirred for 1 h at 100 degrees C. under nitrogen atmosphere. The mixture was allowed to cool down to room temperature. The resulting mixture was extracted with EA. The combined organic layers were washed with brine, dried over anhydrous Na₂SO₄. After filtration, the filtrate was concentrated under reduced pressure. The residue was purified by silica gel column chromatography, eluted with PE:EA (5:1) to afford tert-butyl 7-{[4-(methanesulfonylmethyl)-3-methylphenyl]amino}-1,2,3,4-tetrahydro-2,6-naphthyridine-2-carboxylate as a yellow solid (850 mg; 81.2%).

N-[4-(methanesulfonylmethyl)-3-methylphenyl]-5,6,7,8-tetrahydro-2,6-naphthyridin-3-amine: To a stirred mixture of tert-butyl 7-{[4-(methanesulfonylmethyl)-3-methylphenyl]amino}-1,2,3,4-tetrahydro-2,6-naphthyridine-2-carboxylate (830 mg; 1.91 mmol.) in DCM (9 ml) was added TFA (3 ml) at 0 degrees C. The resulting mixture was stirred for 1 h at room temperature under nitrogen atmosphere. The resulting mixture was concentrated under reduced pressure. The mixture was basified to pH=9 with Saturated sodium bicarbonate aqueous solution. The resulting mixture was extracted with EA. The combined organic layers were washed with brine, dried over anhydrous Na₂SO₄. After filtration, the filtrate was concentrated under reduced pressure to afford N-[4-(methanesulfonylmethyl)-3-methylphenyl]-5,6,7,8-tetrahydro-2,6-naphthyridin-3-amine as a yellow solid (630 mg; 97.3%).

tert-butyl 7-(7-{[4-(methanesulfonylmethyl)-3-methylphenyl]amino}-1,2,3,4-tetrahydro-2,6-naphthyridin-2-yl)-8-methyl-1H,2H,3H-pyrido[2,3-b][1,4]oxazine-1-carboxylate: To a stirred mixture of N-[4-(methanesulfonylmethyl)-3-methylphenyl]-5,6,7,8-tetrahydro-2,6-naphthyridin-3-amine (300 mg; 0.89 mmol) and tert-butyl 7-bromo-8-methyl-1H,2H,3H-pyrido[2,3-b][1,4]oxazine-1-carboxylate (368 mg; 1.06 mmol) in Dioxane-1,4 (5 ml) were added Pd-PEPPSI-IPentCl 2-methylpyridine (o-picoline) (78 mg; 0.09 mmol) and Cs₂CO₃ (608 mg; 1.77 mmol) at room temperature. The resulting mixture was stirred for 1 h at 100 degrees C. under nitrogen atmosphere. The mixture was allowed to cool down to room temperature. The resulting mixture was extracted with EA. The combined organic layers were washed with brine, dried over anhydrous Na₂SO₄. After filtration, the filtrate was concentrated under reduced pressure. The residue was purified by reverse flash chromatography with the following conditions: column, C18 silica gel; mobile phase, MeOH in water, 45% to 50% gradient in 30 min; detector, UV 254 nm to afford tert-butyl 7-(7-{[4-(methanesulfonylmethyl)-3-methylphenyl]amino}-1,2,3,4-tetrahydro-2,6-naphthyridin-2-yl)-8-methyl-1H,2H,3H-pyrido[2,3-b][1,4]oxazine-1-carboxylate as a yellow solid (260 mg; 46.0%).

N-[4-(methanesulfonylmethyl)-3-methylphenyl]-6-{8-methyl-1H,2H,3H-pyrido[2,3-b][1,4]oxazin-7-yl}-5,6,7,8-tetrahydro-2,6-naphthyridin-3-amine: To a stirred mixture of tert-butyl 7-(7-{[4-(methanesulfonylmethyl)-3-methylphenyl]amino}-1,2,3,4-tetrahydro-2,6-naphthyridin-2-yl)-8-methyl-1H,2H,3H-pyrido[2,3-b][1,4]oxazine-1-carboxylate (190 mg; 0.30 mmol) in DCM (3 ml) was added TFA (1 ml) at 0 degrees C. The resulting mixture was stirred for 1.5 h at room temperature under nitrogen atmosphere. The resulting mixture was concentrated under reduced pressure. The mixture was basified to pH=9 with Saturated sodium bicarbonate aqueous solution. The resulting mixture was extracted with EA. The combined organic layers were washed with brine, dried over anhydrous Na₂SO₄. The crude product was purified by Prep-HPLC with the following conditions (Column: XBridge Prep OBD C18 Column, 30*150 mm, 5 μm; Mobile Phase A: 10 mmol NH₄HCO₃+0.05% NH₄OH, Mobile Phase B: ACN; Flow rate: 60 mL/min; Gradient: 23% to 50% in 9 min; Wavelength: 254 nm; RT1 (min): 7; Number Of Runs: 1) to afford N-[4-(methanesulfonylmethyl)-3-methylphenyl]-6-{8-methyl-1H,2H,3H-pyrido[2,3-b][1,4]oxazin-7-yl}-5,6,7,8-tetrahydro-2,6-naphthyridin-3-amine as a white solid (22.70 mg; 15.8%).

HPLC: 99.33% purity, 4.49 min. MS: m/z=480 [M+H]⁺. ¹H NMR (300 MHz, DMSO) δ 8.88 (s, 1H), 8.01 (s, 1H), 7.51 (dd, J=8.3, 2.3 Hz, 1H), 7.43 (d, J=2.3 Hz, 1H), 7.27 (s, 1H), 7.19 (d, J=8.4 Hz, 1H), 6.61 (s, 1H), 5.50 (d, J=2.7 Hz, 1H), 4.40 (s, 2H), 4.18 (t, J=4.3 Hz, 2H), 3.99 (s, 2H), 3.31-3.25 (bs, 2H), 3.09 (t, J=5.7 Hz, 2H), 2.94 (s, 3H), 2.82 (t, J=5.7 Hz, 2H), 2.33 (s, 3H), 2.03 (s, 3H).

### Example 180: Synthesis of Compound N-[4-(methanesulfonylmethyl)phenyl]-8-methyl-7-{8-methyl-1H,2H,3H-pyrido[2,3-b][1,4]oxazin-7-yl}-5H,6H,7H,8H-pyrido[3,4-d]pyrimidin-2-amine

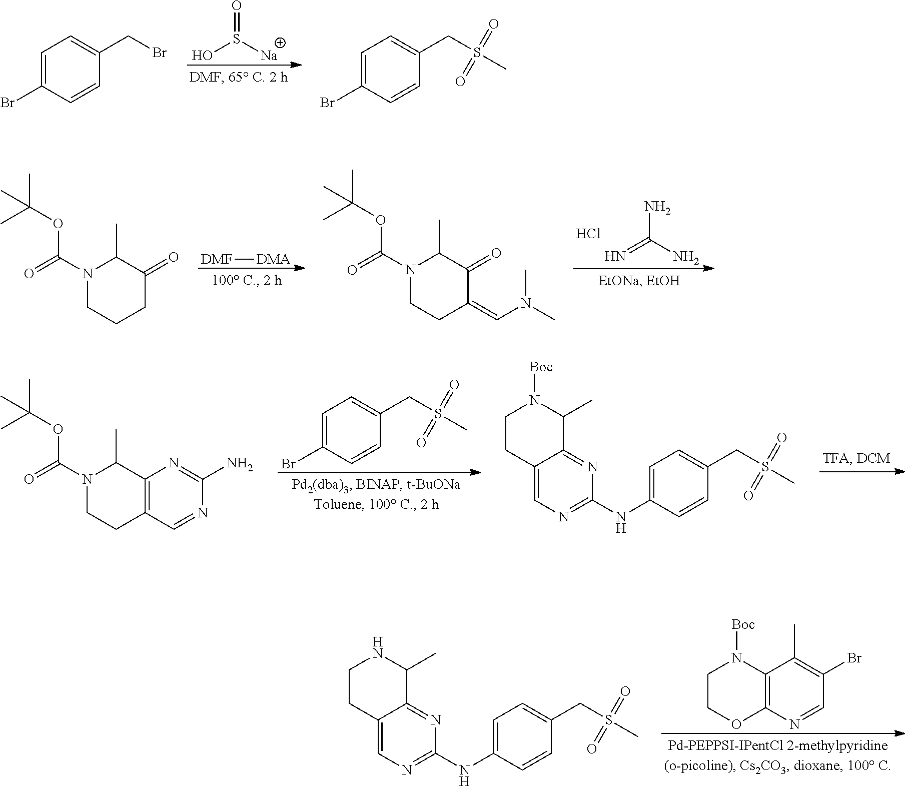

1-bromo-4-(methanesulfonylmethyl)benzene: To a stirred solution of 1-bromo-4-(bromomethyl)benzene (2 g; 7.60 mmol) and DMF (5 ml) was added sodium methanesulfinate (1.57 g; 15.2 mmol). Then the resulting mixture was stirred for additional 2 h at 65 degrees C. The reaction was quenched by the addition of H₂O at room temperature and extracted by EA. The combined organic layers were washed with brine, dried over anhydrous Na₂SO₄. After filtration, the filtrate was concentrated under reduced pressure to afford 1-bromo-4-(methanesulfonylmethyl)benzene as a white solid (1.8 g; 95.0%).

tert-butyl (4Z)-4-[(dimethylamino)methylidene]-2-methyl-3-oxopiperidine-1-carboxylate: To a stirred mixture of tert-butyl 2-methyl-3-oxopiperidine-1-carboxylate (1 g; 4.45 mmol) in DMF-DMA (15 ml) at 25 degrees C. under nitrogen atmosphere. The resulting mixture was stirred for 2 h at 100 degrees C. under nitrogen atmosphere. The resulting mixture was extracted with EA (3×50 mL). The combined organic layers were washed with brine, dried over anhydrous MgSO4. After filtration, the filtrate was concentrated under reduced pressure. The residue was purified by silica gel column chromatography, eluted with EA:PE=60:40 to afford tert-butyl (4Z)-4-[(dimethylamino)methylidene]-2-methyl-3-oxopiperidine-1-carboxylate as a yellow oil (600 mg; 50.2%)

tert-butyl 2-amino-8-methyl-5H,6H,7H,8H-pyrido[3,4-d]pyrimidine-7-carboxylate: To a stirred mixture of tert-butyl (4Z)-4-[(dimethylamino)methylidene]-2-methyl-3-oxopiperidine-1-carboxylate (300 mg; 1.12 mmol) and guanidine hydrochloride (161.81 mg; 1.68 mmol) in EtOH (15 ml) was added EtONa/EtOH(w/w 21%) (1449.06 mg; 4.47 mmol) in portions at 25 degrees C. under nitrogen atmosphere. The resulting mixture was stirred for 20 min at 45 degrees C. and 2 h at 70 degrees C. under nitrogen atmosphere. The resulting mixture was extracted with EA (3×50 mL). The combined organic layers were washed with brine, dried over anhydrous MgSO₄. After filtration, the filtrate was concentrated under reduced pressure. The residue was purified by silica gel column chromatography, eluted with DCM:MEOH=5:95 to afford tert-butyl 2-amino-8-methyl-5H,6H,7H,8H-pyrido[3,4-d]pyrimidine-7-carboxylate as a yellow solid (260 mg; 83.1%)

tert-butyl 2-{[4-(methanesulfonylmethyl)phenyl]amino}-8-methyl-5H,6H,7H,8H-pyrido[3,4-d]pyrimidine-7-carboxylate: To a stirred solution of tert-butyl 2-amino-8-methyl-5H,6H,7H,8H-pyrido[3,4-d]pyrimidine-7-carboxylate (200 mg; 0.71 mmol) and 1-bromo-4-(methanesulfonylmethyl)benzene (282 mg; 1.13 mmol) in Toluene (5 ml) were added Pd₂(dba)₃ (70 mg; 0.07 mmol), [2′-(diphenylphosphanyl)-[1,1′-binaphthalen]-2-yl]diphenylphosphane (96 mg; 0.15 mmol), sodium tert-butoxide (146 mg; 1.44 mmol) in portions at room temperature under N₂ atmosphere. The resulting mixture was stirred for 2 h at 100 degree C. under N₂ atmosphere. The resulting mixture was diluted with water and extracted with EtOAc. The combined organic layers were washed with brine, dried over anhydrous Na₂SO₄. The resulting mixture was concentrated under reduced pressure. The residue was purified by silica gel column, eluted with 70% EtOAc in PE to afford crude product. The crude product was repurified by C18 flash column eluted with 45% ACN in Water to afford tert-butyl 2-{[4-(methanesulfonylmethyl)phenyl]amino}-8-methyl-5H,6H,7H,8H-pyrido[3,4-d]pyrimidine-7-carboxylate as a yellow solid (512 mg; 77.3%)

N-[4-(methanesulfonylmethyl)phenyl]-8-methyl-5H,6H,7H,8H-pyrido[3,4-d]pyrimidin-2-amine: To a stirred solution of tert-butyl 2-{[4-(methanesulfonylmethyl)phenyl]amino}-8-methyl-5H,6H,7H,8H-pyrido[3,4-d]pyrimidine-7-carboxylate (500 mg; 0.98 mmol) in DCM (6 ml) was added TFA (2 ml) dropwise at 0 degree C. under N₂ atmosphere. The resulting mixture was stirred for 2 h at room temperature under N₂ atmosphere. The resulting mixture was diluted with water, adjust PH=10 by saturated NaHCO₃ and extracted with EtOAc. The combined organic layers were washed with brine, dried over anhydrous Na₂SO₄. The resulting mixture was concentrated under reduced pressure. The residue was purified by silica gel column, eluted with 15% EtOAc in PE to afford N-[4-(methanesulfonylmethyl)phenyl]-8-methyl-5H,6H,7H,8H-pyrido[3,4-d]pyrimidin-2-amine as a yellow solid (333 mg; 100%).

tert-butyl 7-(2-{[4-(methanesulfonylmethyl)phenyl]amino}-8-methyl-5H,6H,7H,8H-pyrido[3,4-d]pyrimidin-7-yl)-8-methyl-1H,2H,3H-pyrido[2,3-b][1,4]oxazine-1-carboxylate: To a stirred solution of N-[4-(methanesulfonylmethyl)phenyl]-8-methyl-5H,6H,7H,8H-pyrido[3,4-d]pyrimidin-2-amine (200 mg; 0.59 mmol), Pd-PEPPSI-IPentCl 2-methylpyridine (o-picoline) (51 mg; 0.06 mmol) and CS₂CO₃ (589 mg; 1.72 mmol) in dioxane (3 ml) was added tert-butyl 7-bromo-8-methyl-1H,2H,3H-pyrido[2,3-b][1,4]oxazine-1-carboxylate (237 mg; 0.67 mmol) in portions at room temperature under N₂ atmosphere. The resulting mixture was stirred for 2 h at room 130 degree C. under N₂ atmosphere. The resulting mixture was diluted with water, extracted with EtOAc. The combined organic layers were washed with brine, dried over anhydrous Na₂SO₄. The resulting mixture was concentrated under reduced pressure. The residue was purified by silica gel column, eluted with 30% EtOAc in PE to afford crude product. The crude product was repurified by C18 flash column eluted with 30% ACN in water to afford tert-butyl 7-(2-{[4-(methanesulfonylmethyl)phenyl]amino}-8-methyl-5H,6H,7H,8H-pyrido[3,4-d]pyrimidin-7-yl)-8-methyl-1H,2H,3H-pyrido[2,3-b][1,4]oxazine-1-carboxylate as a yellow solid (48 mg; 14.1%).

N-[4-(methanesulfonylmethyl)phenyl]-8-methyl-7-{8-methyl-1H,2H,3H-pyrido[2,3-b][1,4]oxazin-7-yl}-5H,6H,7H,8H-pyrido[3,4-d]pyrimidin-2-amine: To a stirred solution of tert-butyl 7-(2-{[4-(methanesulfonylmethyl)phenyl]amino}-8-methyl-5H,6H,7H,8H-pyrido[3,4-d]pyrimidin-7-yl)-8-methyl-1H,2H,3H-pyrido[2,3-b][1,4]oxazine-1-carboxylate (38 mg; 0.07 mmol) and DCM (0.5 ml) in was added TFA (0.5 ml) in portions at room temperature under N₂ atmosphere. The resulting mixture was stirred for 2 h at room temperature under N₂ atmosphere. The resulting mixture was diluted with water, adjust PH=10 by saturated NaHCO₃ and extracted with EtOAc. The combined organic layers were washed with brine, dried over anhydrous Na₂SO₄. The resulting mixture was concentrated under reduced pressure. The residue was purified by Prep-HPLC with following condition: Column: XBridge Shield RP18 OBD Column, 30*150 mm, 5 μm; Mobile Phase A: Water (10 mmol/L NH₄HCO₃+0.05% NH₄OH), Mobile Phase B: ACN; Flow rate: 60 mL/min; Gradient: 23% B to 53% B in 9 min; Wavelength: 254 nm; RT1 (min): 7; Number Of Runs: 1) to afford N-[4-(methanesulfonylmethyl)phenyl]-8-methyl-7-{8-methyl-1H,2H,3H-pyrido[2,3-b][1,4]oxazin-7-yl}-5H,6H,7H,8H-pyrido[3,4-d]pyrimidin-2-amine as a white solid (4.60 mg; 14.3%)

HPLC: 97.72% purity, 3.95 min. MS: m/z=481 [M+H]⁺ ¹H NMR (300 MHz, Methanol-d4) δ 8.24 (s, 1H), 7.81-7.72 (m, 2H), 7.40-7.30 (m, 2H), 7.27 (s, 1H), 4.37-4.26 (m, 4H), 4.24 (q, J=6.7 Hz, 1H), 3.42 (t, J=4.5 Hz, 2H), 3.30-3.26 (m, 1H), 3.10 (dt, J=11.2, 5.2 Hz, 1H), 2.87-2.67 (m, 5H), 2.14 (s, 3H), 1.35 (d, J=6.7 Hz, 3H).

### Example 181: Synthesis of Compound 1-(3-methoxyazetidin-1-yl)-2-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}ethan-1-one

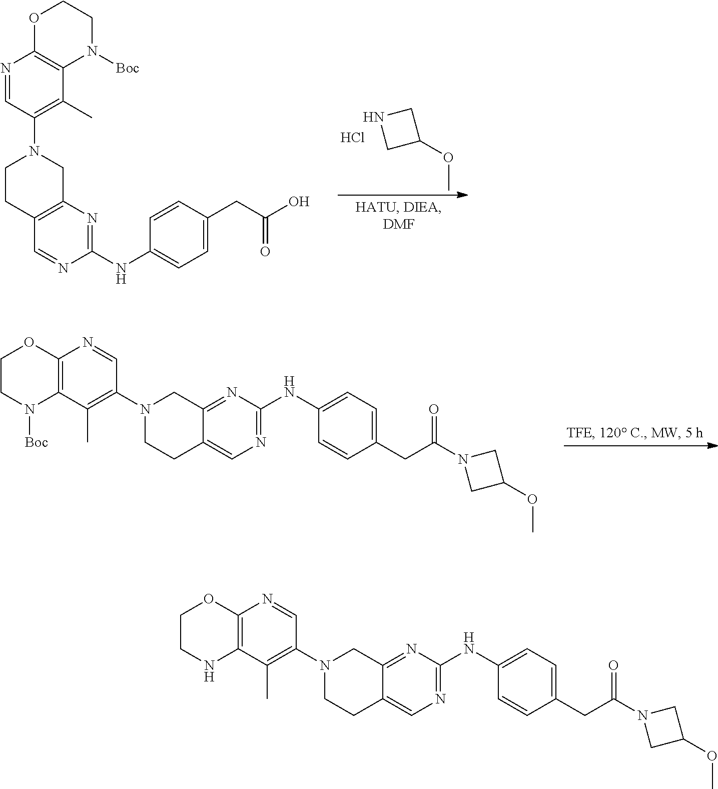

tert-butyl 7-[2-({4-[2-(3-methoxyazetidin-1-yl)-2-oxoethyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate: To a stirred solution of 3-methoxyazetidine hydrochloride (62 mg; 0.48 mmol) and DMF (5 ml) in DIEA (0.29 ml; 1.60 mmol). The resulting mixture was stirred for 10 min at room temperature. Then was added 2-{4-[(7-{1-[(tert-butoxy)carbonyl]-8-methyl-1H,2H,3H-pyrido[2,3-b][1,4]oxazin-7-yl}-5H,6H,7H,8H-pyrido[3,4-d]pyrimidin-2-yl)amino]phenyl}acetic acid (180 mg; 0.32 mmol) and HATU (257 mg; 0.64 mmol) in portions at room temperature under N₂ atmosphere. The resulting mixture was stirred for 2 h at 100 degree C. under N₂ atmosphere. The resulting mixture was diluted with water, extracted with EtOAc. The combined organic layers were washed with brine, dried over anhydrous Na₂SO₄. The resulting mixture was concentrated under reduced pressure. The residue was purified by silica gel column, eluted with 15% EtOH in DCM to afford tert-butyl 7-[2-({4-[2-(3-methoxyazetidin-1-yl)-2-oxoethyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate as a yellow solid (176 mg; 89.3%).

1-(3-methoxyazetidin-1-yl)-2-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}ethan-1-one: To a stirred solution of tert-butyl 7-[2-({4-[2-(3-methoxyazetidin-1-yl)-2-oxoethyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate (150 mg; 0.25 mmol) was added TFE (5 ml) dropwise at room temperature under N₂ atmosphere. The resulting mixture was stirred for 4 h under MW at 120 degree C. under N₂ atmosphere. The resulting mixture was diluted with water, extracted with EtOAc. The combined organic layers were washed with brine, dried over anhydrous Na₂SO₄. The resulting mixture was concentrated under reduced pressure. The residue was purified by Prep-HPLC with following condition: Halo C18 4.6*100 mm, 2.7 um; Column Oven: 40C; Solvent A: Water/0.05% TFA, Solvent B: ACN/0.05% TFA; Flow: 1.5 mL/min; Gradient: 10% B to 95% B in 8 min, hold 2 min; 254 nm to afford 1-(3-methoxyazetidin-1-yl)-2-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}ethan-1-one as a white solid (61.50 mg; 49.0%).

HPLC: 98.36% purity, 3.81 min. MS: m/z=502 [M+H]⁺. ¹H NMR (300 MHz, DMSO-d6) δ 9.47 (s, 1H), 8.32 (s, 1H), 7.67 (dd, J=8.6, 1.7 Hz, 2H), 7.28 (s, 1H), 7.14-7.06 (m, 2H), 5.53 (s, 1H), 4.37-4.26 (m, 1H), 4.17 (ddd, J=10.4, 6.9, 4.0 Hz, 3H), 4.06-3.90 (m, 4H), 3.62 (dd, J=10.5, 3.9 Hz, 1H), 3.32 (s, 4H), 3.19 (s, 3H), 3.12 (t, J=5.7 Hz, 2H), 2.79 (s, 2H), 2.04 (s, 3H)

### Example 182: 2-methyl-2-(4-((7-(8-methyl-2,3-dihydro-1H-pyrido[2,3-b][1,4]oxazin-7-yl)-5,6,7,8-tetrahydropyrido[3,4-d]pyrimidin-2-yl)amino)phenyl)propanenitrile

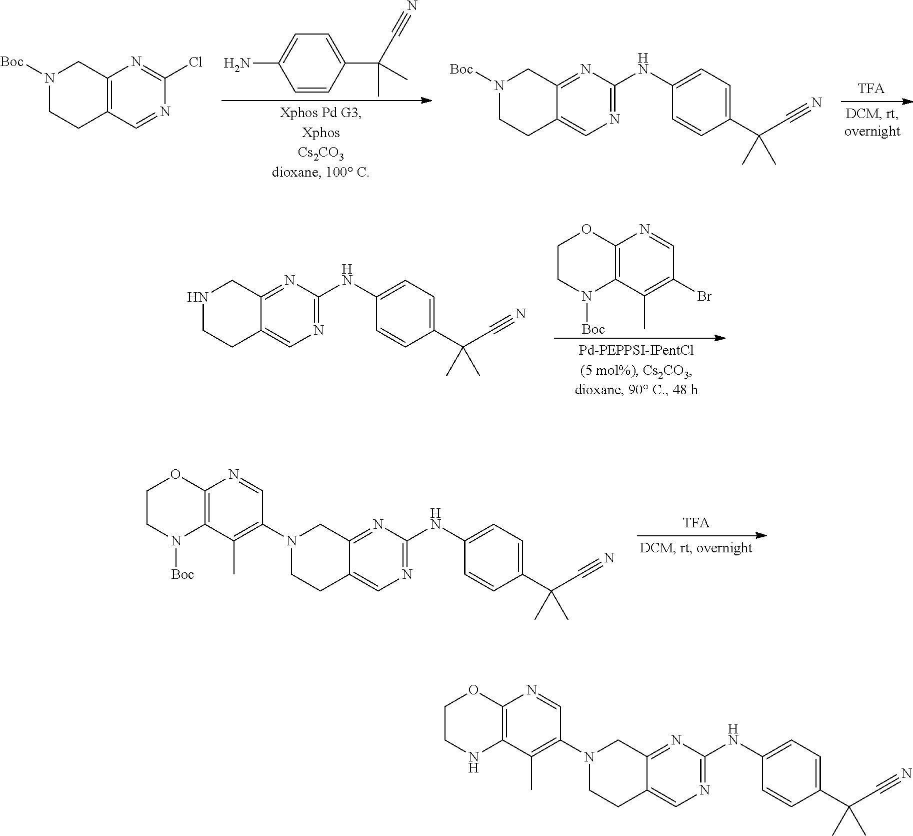

Tert-butyl 2-((4-(2-cyanopropan-2-yl)phenyl)amino)-5,8-dihydropyrido[3,4-d]pyrimidine-7(6H)-carboxylate: To a vial was added tert-butyl 2-chloro-5,8-dihydropyrido[3,4-d]pyrimidine-7(6H)-carboxylate (100 mg; 0.371 mmol; 1.00 eq.), 2-(4-Aminophenyl)-2-methylpropanenitrile (89. mg; 0.556 mmol; 1.50 eq.), Xphos Pd G3 (16.5 mg; 0.019 mmol; 0.05 eq.), Xphos (18.6 mg; 0.037 mmol; 0.10 eq.), potassium carbonate (102 mg; 0.741 mmol; 2.00 eq.) and dioxane (1.00 ml; 10.00 V). The mixture was heated to 110° C. under Ar for 2 h. The mixture was cooled to room temp and purified by flash chromatography to give the desired product tert-butyl 2-((4-(2-cyanopropan-2-yl)phenyl)amino)-5,8-dihydropyrido[3,4-d]pyrimidine-7(6H)-carboxylate (125 mg; 0.318 mmol; 86%).

2-methyl-2-(4-((5,6,7,8-tetrahydropyrido[3,4-d]pyrimidin-2-yl)amino)phenyl) propanenitrile: To a stirred solution of tert-butyl 2-{[4-(1-cyano-1-methylethyl)phenyl]amino}-5H,6H,7H,8H-pyrido[3,4-d]pyrimidine-7-carboxylate (125 mg; 0.318 mmol; 1.00 eq.) in DCM (5.00 ml) was added trifluoroacetic acid (2.00 ml). The mixture was stirred at room temp for 30 min. The solvent was removed. The residue was dissolved in EtOAc. The solution was neutralized with NaHCO₃, separated, dried over Na₂SO₄ and concentrated. The residue was purified by flash chromatography to give desired product 2-methyl-2-(4-((5,6,7,8-tetrahydropyrido[3,4-d]pyrimidin-2-yl)amino)phenyl) propanenitrile (85 mg, 0.243 mmol, 91%).

tert-butyl 7-(2-((4-(2-cyanopropan-2-yl)phenyl)amino)-5,8-dihydropyrido[3,4-d]pyrimidin-7(6H)-yl)-8-methyl-2,3-dihydro-1H-pyrido[2,3-b][1,4]oxazine-1-carboxylate: To a vial was added 2-methyl-2-[4-({5H,6H,7H,8H-pyrido[3,4-d]pyrimidin-2-yl}amino)phenyl]propanenitrile (89 mg; 0.304 mmol; 1.25 eq.), tert-butyl 7-bromo-8-methyl-1H,2H,3H-pyrido[2,3-b][1,4]oxazine-1-carboxylate (80 mg; 0.243 mmol; 1.00 eq.), PEPPSI IHept-Cl (11.8 mg; 0.012 mmol; 0.05 eq.), cesium carbonate (158 mg; 0.486 mmol; 2.00 eq.) and dioxane (1.20 ml; 15.00 V). The mixture was heated to 110° C. under N₂ for 20 h. The mixture was cooled to room temp and purified directly by flash chromatography to give desired product tert-butyl 7-(2-((4-(2-cyanopropan-2-yl)phenyl)amino)-5,8-dihydropyrido[3,4-d]pyrimidin-7(6H)-yl)-8-methyl-2,3-dihydro-1H-pyrido[2,3-b][1,4]oxazine-1-carboxylate (80 mg; 0.15 mmol; 61%).

2-methyl-2-(4-((7-(8-methyl-2,3-dihydro-1H-pyrido[2,3-b][1,4]oxazin-7-yl)-5,6,7,8-tetrahydropyrido[3,4-d]pyrimidin-2-yl)amino)phenyl)propanenitrile: To a stirred solution of tert-butyl 7-(2-{[4-(1-cyano-1-methylethyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (80 mg; 0.148 mmol; 1.00 eq.) in DCM (5.0 mL) was added trifluoroacetic acid (2.00 ml). The mixture was stirred at room temp for 15 min. The mixture was concentrated and diluted with water. The mixture was neutralized with NaHCO₃, extracted with EtOAc, dried over Na₂SO₄ and concentrated. The residue was purified by reverse phase HPLC to give the product 2-methyl-2-(4-((7-(8-methyl-2,3-dihydro-1H-pyrido[2,3-b][1,4]oxazin-7-yl)-5,6,7,8-tetrahydropyrido[3,4-d]pyrimidin-2-yl)amino)phenyl)propanenitrile (17 mg; 0.04 mmol; 26%).

LCMS (LCMS-2): [M+H] for C₂₅H₂₈N₇O calculated: 442.2, found: 442.2. HPLC (HPLC-2): 99% pure (254 nm) at 0.73 minutes. ¹H NMR (500 MHz, DMSO): δ 9.64 (s, 1H), 8.34 (s, 1H), 7.78 (d, J=8.4 Hz, 2H), 7.39 (d, J=8.5 Hz, 2H), 7.28 (s, 1H), 5.56 (s, 1H), 4.19 (t, J=4.4 Hz, 2H), 3.96 (s, 2H), 3.29 (d, J=5.5 Hz, 2H), 3.11 (t, J=5.7 Hz, 2H), 2.79 (t, J=5.7 Hz, 2H), 2.03 (s, 3H), 1.65 (s, 6H).

### Example 183: Synthesis of 7-{8-methyl-1H,2H,3H-pyrido[2,3-b][1,4]oxazin-7-yl}-N-{4-[(1H-1,2,3-triazol-1-yl)methyl]phenyl}-5H,6H,7H,8H-pyrido[3,4-d]pyrimidin-2-amine

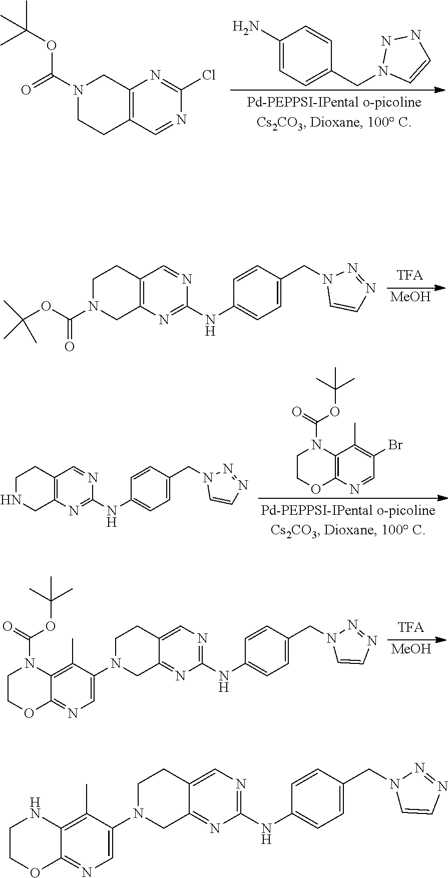

tert-butyl 2-({4-[(1H-1,2,3-triazol-1-yl)methyl]phenyl}amino)-5H,6H,7H,8H-pyrido[3,4-d]pyrimidine-7-carboxylate: A suspension of tert-butyl 2-chloro-5H,6H,7H,8H-pyrido[3,4-d]pyrimidine-7-carboxylate (0.70 g; 2.60 mmol; 1.00 eq.), 4-[(1H-1,2,3-triazol-1-yl)methyl]aniline (0.54 g; 3.11 mmol; 1.20 eq.), cesium carbonate (2.54 g; 7.79 mmol; 3.00 eq.) in dioxane (20 mL) was degassed with Argon for 5 minutes, then Pd-PEPPSI-iPent-Cl O-picoline (126.23 mg; 0.13 mmol; 0.05 eq.) was added and capped. The mixture was heated at 100° C. for 16 h LCMS showed the reaction was complete and desired product was observed. The insoluble was removed by filtration. The solvent was concentrated and to get tert-butyl 2-({4-[(1H-1,2,3-triazol-1-yl)methyl]phenyl}amino)-5H,6H,7H,8H-pyrido[3,4-d]pyrimidine-7-carboxylate (900.00 mg; 2.21 mmol in 85.1% yield) as pale yellow powder, used as is in the next step. MS: m/z 408.20 (M+H).

N-{4-[(1H-1,2,3-triazol-1-yl)methyl]phenyl}-5H,6H,7H,8H-pyrido[3,4-d]pyrimidin-2-amine: To a suspension of tert-butyl 2-({4-[(1H-1,2,3-triazol-1-yl)methyl]phenyl}amino)-5H,6H,7H,8H-pyrido[3,4-d]pyrimidine-7-carboxylate (9000.00 mg; 22.09 mmol; 1.00 eq.) in MeOH (3 mL) was added trifluoroacetic acid (126.77 ml; 1656.57 mmol; 75.00 eq.). The mixture was stirred for 1 hour. LC/MS showed the reaction was complete. After removal of the solvents with Rotavapor, the residue was dissolved in DMF (20 mL) and the mixture was purified through InterChim reverse phase with Acetonitrile in 0.1 NH₄OH from 0% to 100% in 14 minutes with a flow rate of 25 mL/min to provide the desired product N-{4-[(1H-1,2,3-triazol-1-yl)methyl]phenyl}-5H,6H,7H,8H-pyrido[3,4-d]pyrimidin-2-amine (250 mg. 33.2% yield). MS: m/z 308.20 (M+H).

Tert-butyl 8-methyl-7-[2-({4-[(1H-1,2,3-triazol-1-yl)methyl]phenyl}amino)-5H,6H,7H,8H-pyrido[3,4-d]pyrimidin-7-yl]-1H,2H,3H-pyrido[2,3-b][1,4]oxazine-1-carboxylate: To a stirred solution of N-{4-[(1H-1,2,3-triazol-1-yl)methyl]phenyl}-5H,6H,7H,8H-pyrido[3,4-d]pyrimidin-2-amine (200.00 mg; 0.65 mmol; 1.00 eq.) and tert-butyl 7-bromo-8-methyl-1H,2H,3H-pyrido[2,3-b][1,4]oxazine-1-carboxylate (247.80 mg; 0.72 mmol; 1.10 eq.) in was added Pd-PEPPSI-IPentCl 2-methylpyridine (o-picoline) (28.77 mg; 0.03 mmol; 0.05 eq.) and cesium carbonate (446.35 mg; 1.30 mmol; 2.00 eq.) in 1,4-dioxane (100 mL). The resulting mixture was stirred for overnight at 100 degrees C. under nitrogen atmosphere. LCMS showed the reaction was complete and desired product was observed. The insoluble was removed by filtration. The solvent was concentrated. The mixture was loaded at InterChim and purified (0.1% formic acid in water and acetonitrile) to get tert-butyl 8-methyl-7-[2-({4-[(1H-1,2,3-triazol-1-yl)methyl]phenyl}amino)-5H,6H,7H,8H-pyrido[3,4-d]pyrimidin-7-yl]-1H,2H,3H-pyrido[2,3-b][1,4]oxazine-1-carboxylate (170.00 mg; 0.31 mmol, 47% yield). MS: m/z 556.30 (M+H)

7-{8-methyl-1H,2H,3H-pyrido[2,3-b][1,4]oxazin-7-yl}-N-{4-[(1H-1,2,3-triazol-1-yl)methyl]phenyl}-5H,6H,7H,8H-pyrido[3,4-d]pyrimidin-2-amine: To a suspension of tert-butyl 8-methyl-7-[2-({4-[(1H-1,2,3-triazol-1-yl)methyl]phenyl}amino)-5H,6H,7H,8H-pyrido[3,4-d]pyrimidin-7-yl]-1H,2H,3H-pyrido[2,3-b][1,4]oxazine-1-carboxylate (170.00 mg; 0.31 mmol; 1.00 eq.) in MeOH (1 mL) was added trifluoroacetic acid (1.76 ml; 22.95 mmol; 75.00 eq.). The mixture was stirred at for 15 minutes. LC/MS showed the reaction was complete. After removal of the solvents with Rotavapor, the residue was dissolved in water (10 mL) and adjusted pH to 12-14 with addition of aq NaOH (2N). The precipitated solid was filtered and washed with water three time. The solid was dissolved in DMF and loaded at InterChim to purify at basic condition (0.1% NH₄OH in water and acetonitrile) to provide 7-{8-methyl-1H,2H,3H-pyrido[2,3-b][1,4]oxazin-7-yl}-N-{4-[(1H-1,2,3-triazol-1-yl)methyl]phenyl}-5H,6H,7H,8H-pyrido[3,4-d]pyrimidin-2-amine. MS: m/z 456 (M+H). ¹H NMR (500 MHz, DMSO) δ 9.62 (s, 1H), 8.34 (s, 1H), 8.15 (s, 1H), 7.74 (d, J=9.2 Hz, 2H), 7.39-7.20 (m, 2H), 5.71 (s, 1H), 5.52 (s, 2H), 4.23 (t, J=4.4 Hz, 2H), 3.97 (s, 2H), 3.32 (t, J=4.4 Hz, 2H), 3.12 (d, J=11.6 Hz, 2H), 2.79 (t, J=5.7 Hz, 2H), 2.05 (s, 3H).

### Example 185: Synthesis of 7-{8-methyl-1H,2H,3H-pyrido[2,3-b][1,4]oxazin-7-yl}-N-{4-[(2H-1,2,3-triazol-2-yl)methyl]phenyl}-5H,6H,7H,8H-pyrido[3,4-d]pyrimidin-2-amine

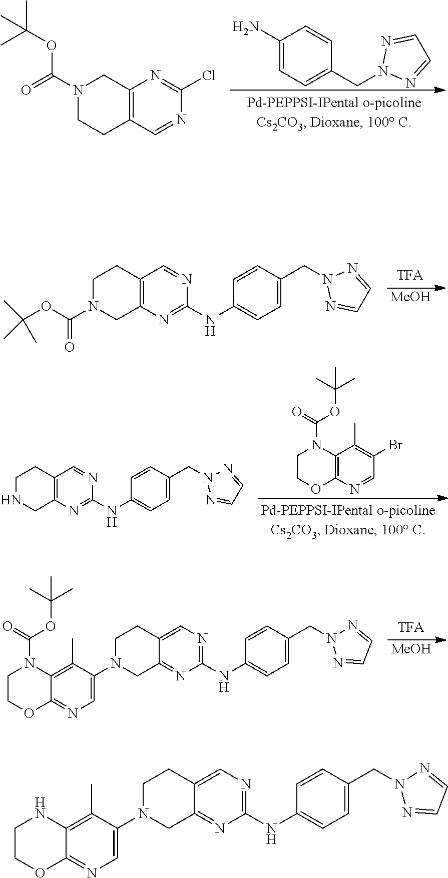

tert-butyl 2-({4-[(2H-1,2,3-triazol-2-yl)methyl]phenyl}amino)-5H,6H,7H,8H-pyrido[3,4-d]pyrimidine-7-carboxylate: In a 40 mL vial were added tert-butyl 2-chloro-5H,6H,7H,8H-pyrido[3,4-d]pyrimidine-7-carboxylate (0.70 g; 2.60 mmol; 1.00 eq.), 4-[(2H-1,2,3-triazol-2-yl)methyl]aniline (0.54 g; 3.11 mmol; 1.20 eq.), CESIUM CARBONATE (2.54 g; 7.79 mmol; 3.00 eq.) in dioxane (20 mL) to give a suspension. The brown mixture was degassed with Argon for 5 minutes, then 2-methylpyridine; {1,3-bis[2,6-bis(pentan-3-yl)phenyl]-4,5-dichloro-2,3-dihydro-1H-imidazol-2-ylidene}dichloropalladium (109.02 mg; 0.13 mmol; 0.05 eq.) was added and capped. The mixture was heated at 100° C. for 16 h. LCMS showed the reaction was complete and desired product was observed. The insoluble was removed by filtration. The solvent was concentrated and to get tert-butyl 2-({4-[(2H-1,2,3-triazol-2-yl)methyl]phenyl}amino)-5H,6H,7H,8H-pyrido[3,4-d]pyrimidine-7-carboxylate (900.00 mg; 2.21 mmol, yield 85%) as pale yellow powder, used as is in the next step. MS: m/z 408 (M+H).

N-{4-[(2H-1,2,3-triazol-2-yl)methyl]phenyl}-5H,6H,7H,8H-pyrido[3,4-d]pyrimidin-2-amine: To a suspension of tert-butyl 2-({4-[(2H-1,2,3-triazol-2-yl)methyl]phenyl}amino)-5H,6H,7H,8H-pyrido[3,4-d]pyrimidine-7-carboxylate (700.00 mg; 1.72 mmol; 1.00 eq.) in MeOH (3 mL) was added trifluoroacetic acid (9.86 ml; 128.84 mmol; 75.00 eq.). The mixture was stirred at for 1 hour. LC/MS showed the reaction was complete. After removal of the solvents with Rotavapor, the residue was dissolved in DMF (20 mL) and the mixture was purified through InterChim reverse phase with Acetonitrile in 0.1 NH₄OH from 0% to 100% in 14 minutes with a flow rate of 25 mL/min to provide the desired product N-{4-[(2H-1,2,3-triazol-2-yl)methyl]phenyl}-5H,6H,7H,8H-pyrido[3,4-d]pyrimidin-2-amine (470.00 mg; 1.53 mmol, yield 44%). MS: m/z 308 (M+H).

tert-butyl 8-methyl-7-[2-({4-[(2H-1,2,3-triazol-2-yl)methyl]phenyl}amino)-5H,6H,7H,8H-pyrido[3,4-d]pyrimidin-7-yl]-1H,2H,3H-pyrido[2,3-b][1,4]oxazine-1-carboxylate: To a stirred solution of N-{4-[(2H-1,2,3-triazol-2-yl)methyl]phenyl}-5H,6H,7H,8H-pyrido[3,4-d]pyrimidin-2-amine (200.00 mg; 0.65 mmol; 1.00 eq.) and tert-butyl 7-bromo-8-methyl-1H,2H,3H-pyrido[2,3-b][1,4]oxazine-1-carboxylate (247.80 mg; 0.72 mmol; 1.10 eq.) in was added Pd-PEPPSI-IPentCl 2-methylpyridine (o-picoline) (28.77 mg; 0.03 mmol; 0.05 eq.) and cesium carbonate (446.35 mg; 1.30 mmol; 2.00 eq.) in 1,4-dioxane (15 mL). The resulting mixture was stirred for overnight at 100 degrees C. under nitrogen atmosphere. LCMS showed the reaction was complete and desired product was observed. The insoluble was removed by filtration. The solvent was concentrated. The mixture was loaded at InterChim to purified at basic condition (0.1% formic acid in water and acetonitrile) get tert-butyl 8-methyl-7-[2-({4-[(2H-1,2,3-triazol-2-yl)methyl]phenyl}amino)-5H,6H,7H,8H-pyrido[3,4-d]pyrimidin-7-yl]-1H,2H,3H-pyrido[2,3-b][1,4]oxazine-1-carboxylate (160.00 mg; 0.29 mmol, 44% yield). MS: m/z 556 (M+H).

7-{8-methyl-1H,2H,3H-pyrido[2,3-b][1,4]oxazin-7-yl}-N-{4-[(2H-1,2,3-triazol-2-yl)methyl]phenyl}-5H,6H,7H,8H-pyrido[3,4-d]pyrimidin-2-amine: To a suspension of tert-butyl 8-methyl-7-[2-({4-[(2H-1,2,3-triazol-2-yl)methyl]phenyl}amino)-5H,6H,7H,8H-pyrido[3,4-d]pyrimidin-7-yl]-1H,2H,3H-pyrido[2,3-b][1,4]oxazine-1-carboxylate (160.00 mg; 0.29 mmol; 1.00 eq.) in MeOH (1 mL) was added trifluoroacetic acid (1.65 ml; 21.60 mmol; 75.00 eq.). The mixture was stirred at for 15 minutes. LC/MS showed the reaction was complete. After removal of the solvents with Rotavapor, the residue was dissolved in water (10 mL) and adjusted pH to 12-14 with addition of aq NaOH (2N). The precipitated solid was filtered and washed with water three time. The solid was dissolved in DMF and loaded at InterChim to purify at basic condition (0.1% NH4OH in water and acetonitrile) to provide 7-{8-methyl-1H,2H,3H-pyrido[2,3-b][1,4]oxazin-7-yl}-N-{4-[(2H-1,2,3-triazol-2-yl)methyl]phenyl}-5H,6H,7H,8H-pyrido[3,4-d]pyrimidin-2-amine (18.30 mg, yield 14%). MS: m/z 456 (M+H). ¹H NMR (500 MHz, DMSO) δ 9.60 (s, 1H), 8.33 (s, 1H), 7.79 (s, 2H), 7.72 (d, J=8.2 Hz, 2H), 7.28 (s, 1H), 7.19 (d, J=8.3 Hz, 2H), 5.55 (s, 3H), 4.19 (t, J=4.4 Hz, 2H), 3.95 (s, 2H), 3.30 (q, J=3.7 Hz, 2H), 3.11 (t, J=5.8 Hz, 2H), 2.79 (t, J=5.8 Hz, 2H), 2.03 (s, 3H).

### Example 186: Synthesis of N-[4-(2-methanesulfonylpropan-2-yl)phenyl]-7-{8-methyl-1H,2H,3H-pyrido[2,3-b][1,4]oxazin-7-yl}-5H,6H,7H,8H-pyrido[3,4-d]pyrimidin-2-amine

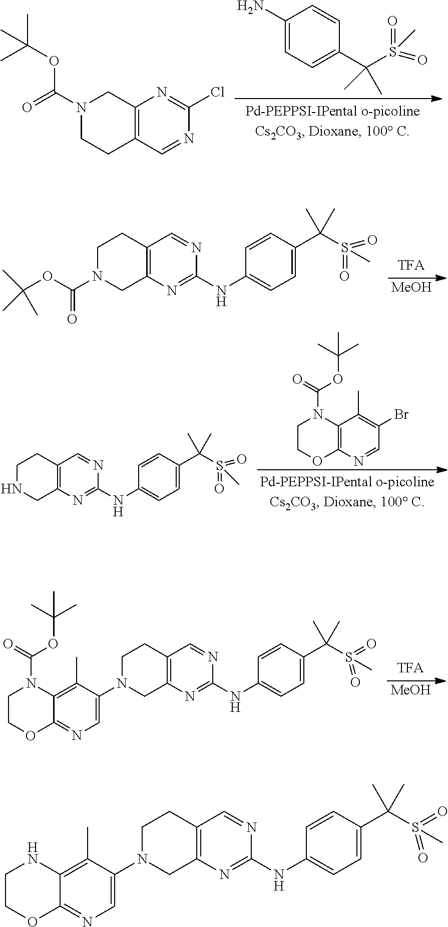

tert-butyl 2-{[4-(2-methanesulfonylpropan-2-yl)phenyl]amino}-5H,6H,7H,8H-pyrido[3,4-d]pyrimidine-7-carboxylate: In a 40 mL vial were added tert-butyl 2-chloro-5H,6H,7H,8H-pyrido[3,4-d]pyrimidine-7-carboxylate (0.70 g; 2.60 mmol; 1.00 eq.), 4-(2-methanesulfonylpropan-2-yl)aniline (0.66 g; 3.11 mmol; 1.20 eq.), cesium carbonate (2.54 g; 7.79 mmol; 3.00 eq.) in dioxane (20 mL) to give a suspension. The brown mixture was degassed with Argon for 5 minutes, then Pd-PEPPSI-iHept-o-picoline (109.02 mg; 0.13 mmol; 0.05 eq.) was added and capped. The mixture was heated at 100° C. for 16 h. LCMS showed the reaction was complete and desired product was observed. The insoluble was removed by filtration. The solvent was concentrated and to get tert-butyl 2-{[4-(2-methanesulfonylpropan-2-yl)phenyl]amino}-5H,6H,7H,8H-pyrido[3,4-d]pyrimidine-7-carboxylate (900.00 mg; 2.02 mmol, 77% yield) as pale yellow powder, used as is in the next step. MS: m/z 447 (M+H).

N-[4-(2-methanesulfonylpropan-2-yl)phenyl]-5H,6H,7H,8H-pyrido[3,4-d]pyrimidin-2-amine: To a suspension of tert-butyl 2-{[4-(2-methanesulfonylpropan-2-yl)phenyl]amino}-5H,6H,7H,8H-pyrido[3,4-d]pyrimidine-7-carboxylate (9000.00 mg; 20.15 mmol; 1.00 eq.) in MeOH (3 mL) was added trifluoroacetic acid (115.67 ml; 1511.54 mmol; 75.00 eq.). The mixture was stirred for 1 hour. LC/MS showed the reaction was complete. After removal of the solvents with Rotavapor, the residue was dissolved in DMF (20 mL) and the mixture was purified through InterChim reverse phase with Acetonitrile in 0.1 NH₄OH from 0% to 100% in 14 minutes with a flow rate of 25 mL/min to provide the desired product N-[4-(2-methanesulfonylpropan-2-yl)phenyl]-5H,6H,7H,8H-pyrido[3,4-d]pyrimidin-2-amine (150 mg, 2.1% yield). MS: m/z 347 (M+H).

tert-butyl 7-(2-{[4-(2-methanesulfonylpropan-2-yl)phenyl]amino}-5H,6H,7H,8H-pyrido[3,4-d]pyrimidin-7-yl)-8-methyl-1H,2H,3H-pyrido[2,3-b][1,4]oxazine-1-carboxylate: To a stirred solution of N-[4-(2-methanesulfonylpropan-2-yl)phenyl]-5H,6H,7H,8H-pyrido[3,4-d]pyrimidin-2-amine (150.00 mg; 0.43 mmol; 1.00 eq.) and tert-butyl 7-bromo-8-methyl-1H,2H,3H-pyrido[2,3-b][1,4]oxazine-1-carboxylate (164.88 mg; 0.48 mmol; 1.10 eq.) in was added Pd-PEPPSI-IPentCl 2-methylpyridine (o-picoline) (19.15 mg; 0.02 mmol; 0.05 eq.) and dicesium(1+) carbonate (296.99 mg; 0.87 mmol; 2.00 eq.) in 1,4-dioxane (15 mL). The resulting mixture was stirred for overnight at 100 degrees C. under nitrogen atmosphere. LCMS showed the reaction was complete and desired product was observed. The insoluble was removed by filtration. The solvent was concentrated. The mixture was loaded at InterChim to purified at acidic condition (0.1% formic acid in water and acetonitrile) get tert-butyl 7-(2-{[4-(2-methanesulfonylpropan-2-yl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (26 mg, 10% yield). MS: m/z 595 (M+H).

N-[4-(2-methanesulfonylpropan-2-yl)phenyl]-7-{8-methyl-1H,2H,3H-pyrido[2,3-b][1,4]oxazin-7-yl}-5H,6H,7H,8H-pyrido[3,4-d]pyrimidin-2-amine: To a suspension of tert-butyl 7-(2-{[4-(2-methanesulfonylpropan-2-yl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (26.00 mg; 0.04 mmol; 1.00 eq.) in MeOH (1 mL) was added trifluoroacetic acid (0.25 ml; 3.28 mmol; 75.00 eq.). The mixture was stirred at for 15 minutes. LC/MS showed the reaction was complete. After removal of the solvents with Rotavapor, the residue was dissolved in water (10 mL) and adjusted pH to 12-14 with addition of aq NaOH (2N). The precipitated solid was filtered and washed with water three time. The solid was dissolved in DMF and loaded at InterChim to purify at basic condition (0.1% NH₄OH in water and acetonitrile) to provide N-[4-(2-methanesulfonylpropan-2-yl)phenyl]-7-{8-methyl-1H,2H,3H-pyrido[2,3-b][1,4]oxazin-7-yl}-5H,6H,7H,8H-pyrido[3,4-d]pyrimidin-2-amine (3.5 mg, 16% yield). MS: m/z 495 (M+H). ¹H NMR (500 MHz, DMSO) δ 9.69 (s, 1H), 8.35 (d, J=3.8 Hz, 1H), 7.78 (d, J=8.4 Hz, 2H), 7.48 (d, J=8.5 Hz, 2H), 7.28 (s, 1H), 5.56 (s, 1H), 4.19 (t, J=4.4 Hz, 2H), 3.98 (s, 2H), 3.12 (t, J=5.7 Hz, 2H), 2.81 (d, J=5.8 Hz, 2H), 2.66 (s, 3H), 2.04 (s, 3H), 1.72 (s, 6H), 1.44 (d, J=9.4 Hz, 2H).

### Example 187: Synthesis of 7-{8-methyl-1H,2H,3H-pyrido[2,3-b][1,4]oxazin-7-yl}-N-{4-[(1H-pyrazol-1-yl)methyl]phenyl}-5H,6H,7H,8H-pyrido[3,4-d]pyrimidin-2-amine

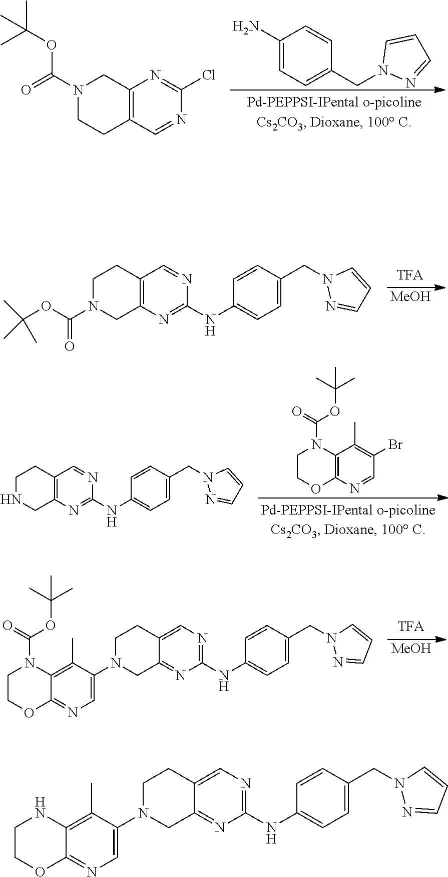

tert-butyl 2-({4-[(1H-pyrazol-1-yl)methyl]phenyl}amino)-5H,6H,7H,8H-pyrido[3,4-d]pyrimidine-7-carboxylate: In a 40 mL vial were added tert-butyl 2-chloro-5H,6H,7H,8H-pyrido[3,4-d]pyrimidine-7-carboxylate (0.70 g; 2.60 mmol; 1.00 eq.), 4-[(1H-pyrazol-1-yl)methyl]aniline (0.54 g; 3.11 mmol; 1.20 eq.), cesium carbonate (2.54 g; 7.79 mmol; 3.00 eq.) in dioxane (20 mL) to give a suspension. The brown mixture was degassed with Argon for 5 minutes, then 2-methylpyridine; {1,3-bis[2,6-bis(pentan-3-yl)phenyl]-4,5-dichloro-2,3-dihydro-1H-imidazol-2-ylidene}dichloropalladium (109.02 mg; 0.13 mmol; 0.05 eq.) was added and capped. The mixture was heated at 100° C. for 16 h. LCMS showed the reaction was complete and desired product was observed. The insoluble was removed by filtration. The solvent was concentrated and to get tert-butyl 2-({4-[(1H-pyrazol-1-yl)methyl]phenyl}amino)-5H,6H,7H,8H-pyrido[3,4-d]pyrimidine-7-carboxylate (900.00 mg; 87% in yield) as pale yellow powder, used as is in the next step. MS: m/z 407 (M+H).

N-{4-[(1H-pyrazol-1-yl)methyl]phenyl}-5H,6H,7H,8H-pyrido[3,4-d]pyrimidin-2-amine: To a suspension of tert-butyl 2-({4-[(1H-pyrazol-1-yl)methyl]phenyl}amino)-5H,6H,7H,8H-pyrido[3,4-d]pyrimidine-7-carboxylate (1100.00 mg; 2.71 mmol; 1.00 eq.) in MeOH (1 mL) was added trifluoroacetic acid (15.53 ml; 202.96 mmol; 75.00 eq.). The mixture was stirred at for 15 minutes. LC/MS showed the reaction was complete. After removal of the solvents with Rotavapor, the residue was dissolved in water (10 mL) and adjusted pH to 12-14 with addition of aq NaOH (2N). The precipitated solid was filtered and washed with water three time. The solid was dissolved in DMF and loaded at InterChim to purify at basic condition (0.1% NH₄OH in water and acetonitrile) to provide N-{4-[(1H-pyrazol-1-yl)methyl]phenyl}-5H,6H,7H,8H-pyrido[3,4-d]pyrimidin-2-amine (740.00 mg; 89% in yield). MS m/z 307 (M+H).

tert-butyl 8-methyl-7-[2-({4-[(1H-pyrazol-1-yl)methyl]phenyl}amino)-5H,6H,7H,8H-pyrido[3,4-d]pyrimidin-7-yl]-1H,2H,3H-pyrido[2,3-b][1,4]oxazine-1-carboxylate: To a stirred solution of N-{4-[(1H-pyrazol-1-yl)methyl]phenyl}-5H,6H,7H,8H-pyrido[3,4-d]pyrimidin-2-amine (200.00 mg; 0.65 mmol; 1.00 eq.) and tert-butyl 7-bromo-8-methyl-1H,2H,3H-pyrido[2,3-b][1,4]oxazine-1-carboxylate (248.60 mg; 0.72 mmol; 1.10 eq.) in was added Pd-PEPPSI-IPentCl 2-methylpyridine (o-picoline) (28.87 mg; 0.03 mmol; 0.05 eq.) and cesium carbonate (447.79 mg; 1.31 mmol; 2.00 eq.) in 1,4-dioxane (15 mL). The resulting mixture was stirred for overnight at 100 degrees C. under nitrogen atmosphere.

LCMS showed the reaction was complete and desired product was observed. The insoluble was removed by filtration. The solvent was concentrated. The mixture was loaded at InterChim to purified at basic condition (0.1% NH₄OH in water and acetonitrile) get tert-butyl 8-methyl-7-[2-({4-[(1H-pyrazol-1-yl)methyl]phenyl}amino)-5H,6H,7H,8H-pyrido[3,4-d]pyrimidin-7-yl]-1H,2H,3H-pyrido[2,3-b][1,4]oxazine-1-carboxylate (110.00 mg; 30% in yield). MS: m/z 555 (M+H).

7-{8-methyl-1H,2H,3H-pyrido[2,3-b][1,4]oxazin-7-yl}-N-{4-[(1H-pyrazol-1-yl)methyl]phenyl}-5H,6H,7H,8H-pyrido[3,4-d]pyrimidin-2-amine: To a suspension of tert-butyl 8-methyl-7-[2-({4-[(1H-pyrazol-1-yl)methyl]phenyl}amino)-5H,6H,7H,8H-pyrido[3,4-d]pyrimidin-7-yl]-1H,2H,3H-pyrido[2,3-b][1,4]oxazine-1-carboxylate (160.00 mg; 0.29 mmol; 1.00 eq.) in MeOH (1 mL) was added trifluoroacetic acid (1.66 ml; 21.64 mmol; 75.00 eq.). The mixture was stirred at for 15 minutes. LC/MS showed the reaction was complete. After removal of the solvents with Rotavapor, the residue was dissolved in water (10 mL) and adjusted pH to 12-14 with addition of aq. NaOH (2N). The precipitated solid was filtered and washed with water three time. The solid was dissolved in DMF and loaded at InterChim to purify at basic condition (0.1% NH₄OH in water and acetonitrile) to provide 7-{8-methyl-1H,2H,3H-pyrido[2,3-b][1,4]oxazin-7-yl}-N-{4-[(1H-pyrazol-1-yl)methyl]phenyl}-5H,6H,7H,8H-pyrido[3,4-d]pyrimidin-2-amine (8.90 mg; yield 6.2%). MS: mz/455 (M+H). ¹H NMR (500 MHz, DMSO) δ 9.60 (s, 1H), 8.33 (s, 1H), 7.79 (s, 2H), 7.72 (d, J=8.3 Hz, 2H), 7.28 (s, 1H), 7.19 (d, J=8.3 Hz, 2H), 5.55 (s, 3H), 4.19 (t, J=4.4 Hz, 2H), 3.95 (s, 2H), 3.30 (q, J=3.8, 3.3 Hz, 2H), 3.11 (t, J=5.8 Hz, 2H), 2.79 (t, J=5.8 Hz, 2H), 2.03 (s, 3H).

### Example 188: Synthesis of 3-({4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}methyl)-1,3-oxazolidin-2-one

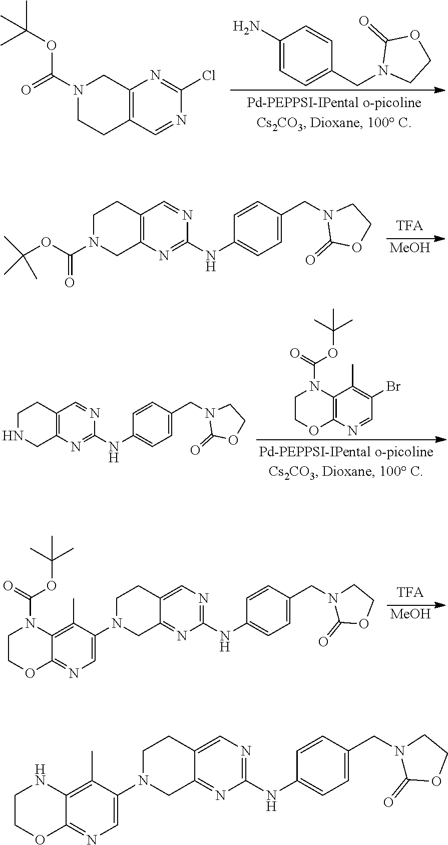

tert-butyl 2-({4-[(2-oxo-1,3-oxazolidin-3-yl)methyl]phenyl}amino)-5H,6H,7H,8H-pyrido[3,4-d]pyrimidine-7-carboxylate: In a 40 mL vial were added tert-butyl 2-chloro-5H,6H,7H,8H-pyrido[3,4-d]pyrimidine-7-carboxylate (0.70 g; 2.60 mmol; 1.00 eq.), 3-[(4-aminophenyl)methyl]-1,3-oxazolidin-2-one (0.60 g; 3.11 mmol; 1.20 eq.), cesium carbonate (2.54 g; 7.79 mmol; 3.00 eq.) in dioxane (20 mL) to give a suspension.

The brown mixture was degassed with Argon for 5 minutes, then 2-methylpyridine; {1,3-bis[2,6-bis(pentan-3-yl)phenyl]-4,5-dichloro-2,3-dihydro-1H-imidazol-2-ylidene}dichloropalladium (109.02 mg; 0.13 mmol; 0.05 eq.) was added and capped. The mixture was heated at 100° C. for 16 h. LCMS showed the reaction was complete and desired product was observed. The insoluble was removed by filtration. The solvent was concentrated and to get tert-butyl 2-({4-[(2-oxo-1,3-oxazolidin-3-yl)methyl]phenyl}amino)-5H,6H,7H,8H-pyrido[3,4-d]pyrimidine-7-carboxylate (1000.00 mg; yield in 90%) as pale yellow powder, used as is in the next step. MS: m/z 426 (M+H).

3-{[4-({5H,6H,7H,8H-pyrido[3,4-d]pyrimidin-2-yl}amino)phenyl]methyl}-1,3-oxazolidin-2-one: To a suspension of tert-butyl 2-({4-[(2-oxo-1,3-oxazolidin-3-yl)methyl]phenyl}amino)-5H,6H,7H,8H-pyrido[3,4-d]pyrimidine-7-carboxylate (1000.00 mg; 2.35 mmol; 1.00 eq.) in MeOH (1 mL) was added trifluoroacetic acid (13.49 ml; 176.27 mmol; 75.00 eq.). The mixture was stirred at for 15 minutes. LC/MS showed the reaction was complete. After removal of the solvents with Rotavapor, the residue was dissolved in water (10 mL) and adjusted pH to 12-14 with addition of aq. NaOH (2N). The precipitated solid was filtered and washed with water three time. The solid was dissolved in DMF and loaded at InterChim to purify at basic condition (0.1% NH₄OH in water and acetonitrile) to provide 3-{[4-({5H,6H,7H,8H-pyrido[3,4-d]pyrimidin-2-yl}amino)phenyl]methyl}-1,3-oxazolidin-2-one (500.00 mg; 65%). MS: m/z 326 (M+H).

tert-butyl 8-methyl-7-[2-({4-[(2-oxo-1,3-oxazolidin-3-yl)methyl]phenyl}amino)-5H,6H,7H,8H-pyrido[3,4-d]pyrimidin-7-yl]-1H,2H,3H-pyrido[2,3-b][1,4]oxazine-1-carboxylate: To a stirred solution of 3-{[4-({5H,6H,7H,8H-pyrido[3,4-d]pyrimidin-2-yl}amino)phenyl]methyl}-1,3-oxazolidin-2-one (200.00 mg; 0.61 mmol; 1.00 eq.) and tert-butyl 7-bromo-8-methyl-1H,2H,3H-pyrido[2,3-b][1,4]oxazine-1-carboxylate (234.08 mg; 0.68 mmol; 1.10 eq.) in was added Pd-PEPPSI-IPentCl 2-methylpyridine (o-picoline) (27.18 mg; 0.03 mmol; 0.05 eq.) and cesium carbonate (421.64 mg; 1.23 mmol; 2.00 eq.) in 1,4-dioxane (15 mL). The resulting mixture was stirred for overnight at 100 degrees C. under nitrogen atmosphere. LCMS showed the reaction was complete and desired product was observed. The insoluble was removed by filtration. The solvent was concentrated. The mixture was loaded at InterChim to purified at basic condition (0.1% NH4OH in water and acetonitrile) get tert-butyl 8-methyl-7-[2-({4-[(2-oxo-1,3-oxazolidin-3-yl)methyl]phenyl}amino)-5H,6H,7H,8H-pyrido[3,4-d]pyrimidin-7-yl]-1H,2H,3H-pyrido[2,3-b][1,4]oxazine-1-carboxylate (190.00 mg; yield in 54%). MS: m/z 574 (M+H).

3-({4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}methyl)-1,3-oxazolidin-2-one: To a suspension of tert-butyl 8-methyl-7-[2-({4-[(2-oxo-1,3-oxazolidin-3-yl)methyl]phenyl}amino)-5H,6H,7H,8H-pyrido[3,4-d]pyrimidin-7-yl]-1H,2H,3H-pyrido[2,3-b][1,4]oxazine-1-carboxylate (190.00 mg; 0.33 mmol; 1.00 eq.) in MeOH (1 mL) was added trifluoroacetic acid (1.90 ml; 24.84 mmol; 75.00 eq.). The mixture was stirred at for 15 minutes. LC/MS showed the reaction was complete. After removal of the solvents with Rotavapor, the residue was dissolved in water (10 mL) and adjusted pH to 12-14 with addition of aq. NaOH (2N). The precipitated solid was filtered and washed with water three time. The solid was dissolved in DMF and loaded at InterChim to purify at basic condition (0.1% NH₄OH in water and acetonitrile) to provide 3-({4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}methyl)-1,3-oxazolidin-2-one (58.10 mg; yield 34%). MS: m/z 473 (M+H). ¹H NMR (500 MHz, DMSO) δ 9.59 (s, 1H), 8.34 (s, 1H), 7.81-7.70 (m, 2H), 7.28 (s, 1H), 7.17 (d, J=8.1 Hz, 2H), 5.55 (d, J=3.8 Hz, 1H), 4.30-4.21 (m, 4H), 4.19 (t, J=4.3 Hz, 2H), 3.96 (s, 2H), 3.39 (t, J=8.0 Hz, 2H), 3.12 (t, J=5.7 Hz, 2H), 2.79 (t, J=5.7 Hz, 2H), 2.04 (s, 3H).

### Example 189: Synthesis of N,N-dimethyl-2-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}acetamide

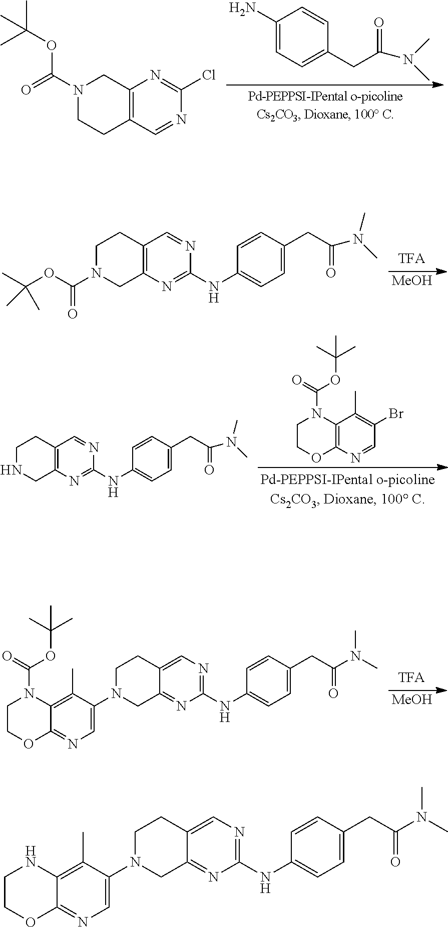

tert-butyl 2-({4-[(dimethylcarbamoyl)methyl]phenyl}amino)-5H,6H,7H,8H-pyrido[3,4-d]pyrimidine-7-carboxylate: In a 40 mL vial were added tert-butyl 2-chloro-5H,6H,7H,8H-pyrido[3,4-d]pyrimidine-7-carboxylate (0.70 g; 2.60 mmol; 1.00 eq.), 2-(4-aminophenyl)-N,N-dimethylacetamide (0.56 g; 3.11 mmol; 1.20 eq.), cesium carbonate (2.54 g; 7.79 mmol; 3.00 eq.) in dioxane (20 mL) to give a suspension. The brown mixture was degassed with Argon for 5 minutes, then 2-methylpyridine; {1,3-bis[2,6-bis(pentan-3-yl)phenyl]-4,5-dichloro-2,3-dihydro-1H-imidazol-2-ylidene}dichloropalladium (109.02 mg; 0.13 mmol; 0.05 eq.) was added and capped. The mixture was heated at 100° C. for 16 h.

LCMS showed the reaction was complete and desired product was observed. The insoluble was removed by filtration. The solvent was concentrated and to get tert-butyl 2-({4-[(dimethylcarbamoyl)methyl]phenyl}amino)-5H,6H,7H,8H-pyrido[3,4-d]pyrimidine-7-carboxylate as pale yellow powder, used in the next step without further purification (900, 84% in yield). MS: m/z 412 (M+H).

N,N-dimethyl-2-[4-({5H,6H,7H,8H-pyrido[3,4-d]pyrimidin-2-yl}amino)phenyl]acetamide: To a suspension of tert-butyl 2-({4-[(dimethylcarbamoyl)methyl]phenyl}amino)-5H,6H,7H,8H-pyrido[3,4-d]pyrimidine-7-carboxylate (900.00 mg; 2.19 mmol; 1.00 eq.) in MeOH (1 mL) was added trifluoroacetic acid (12.55 ml; 164.03 mmol; 75.00 eq.). The mixture was stirred at for 15 minutes. LC/MS showed the reaction was complete. After removal of the solvents with Rotavapor, the residue was dissolved in water (10 mL) and adjusted pH to 12-14 with addition of aq. NaOH (2N). The precipitated solid was filtered and washed with water three time. The solid was dissolved in DMF and loaded at InterChim to purify at basic condition (0.1% NH₄OH in water and acetonitrile) to provide N,N-dimethyl-2-[4-({5H,6H,7H,8H-pyrido[3,4-d]pyrimidin-2-yl}amino)phenyl]acetamide (510.00 mg; 75% in yield). MS: m/z 312.

tert-butyl 7-[2-({4-[(dimethylcarbamoyl)methyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate: To a stirred solution of N,N-dimethyl-2-[4-({5H,6H,7H,8H-pyrido[3,4-d]pyrimidin-2-yl}amino)phenyl]acetamide (200.00 mg; 0.64 mmol; 1.00 eq.) and tert-butyl 7-bromo-8-methyl-1H,2H,3H-pyrido[2,3-b][1,4]oxazine-1-carboxylate (244.59 mg; 0.71 mmol; 1.10 eq.) in was added Pd-PEPPSI-IPentCl 2-methylpyridine (o-picoline) (28.40 mg; 0.03 mmol; 0.05 eq.) and cesium carbonate (440.58 mg; 1.28 mmol; 2.00 eq.) in 1,4-dioxane (15 mL). The resulting mixture was stirred for overnight at 100 degrees C. under nitrogen atmosphere. LCMS showed the reaction was complete and desired product was observed. The insoluble was removed by filtration. The solvent was concentrated. The mixture was loaded at InterChim to purified at basic condition (0.1% NH₄OH in water and acetonitrile) get tert-butyl 7-[2-({4-[(dimethylcarbamoyl)methyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate (160 mg, 44% in yield). MS: 560 (M+H).

N,N-dimethyl-2-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}acetamide: To a suspension of tert-butyl 7-[2-({4-[(dimethylcarbamoyl) methyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate (200.00 mg; 0.36 mmol; 1.00 eq.) in MeOH (1 mL) was added trifluoroacetic acid (2.05 ml; 26.80 mmol; 75.00 eq.). The mixture was stirred for 15 minutes. LC/MS showed the reaction was complete. After removal of the solvents with Rotavapor, the residue was dissolved in DMF (6 mL) and adjusted pH to 12-14 with addition of aq. NaOH (2N). The mixture was loaded at InterChim column to purify at basic condition (0.1% NH4OH in water and acetonitrile) to provide N,N-dimethyl-2-{4-[(7-{8-methyl-1H,2H,3H-pyrido[2,3-b][1,4]oxazin-7-yl}-5H,6H,7H,8H-pyrido[3,4-d]pyrimidin-2-yl)amino]phenyl}acetamide (35.10 mg; 21.4% in yield). MS: m/z 460 (M+H). ¹H NMR (500 MHz, DMSO) δ 9.49 (s, 1H), 8.32 (s, 1H), 7.66 (d, J=8.2 Hz, 2H), 7.28 (s, 1H), 7.09 (d, J=8.1 Hz, 2H), 5.55 (s, 1H), 4.19 (s, 2H), 3.95 (s, 2H), 3.60 (s, 2H), 3.30 (s, 2H), 3.11 (t, J=5.7 Hz, 2H), 2.99 (s, 3H), 2.82 (s, 3H), 2.79 (d, J=6.2 Hz, 2H), 2.04 (s, 3H).

### Example 190: Synthesis of N-[3-fluoro-4-(methanesulfonylmethyl)phenyl]-7-{8-methyl-1H,2H,3H-pyrido[2,3-b][1,4]oxazin-7-yl}-5H,6H,7H,8H-pyrido[3,4-d]pyrimidin-2-amine

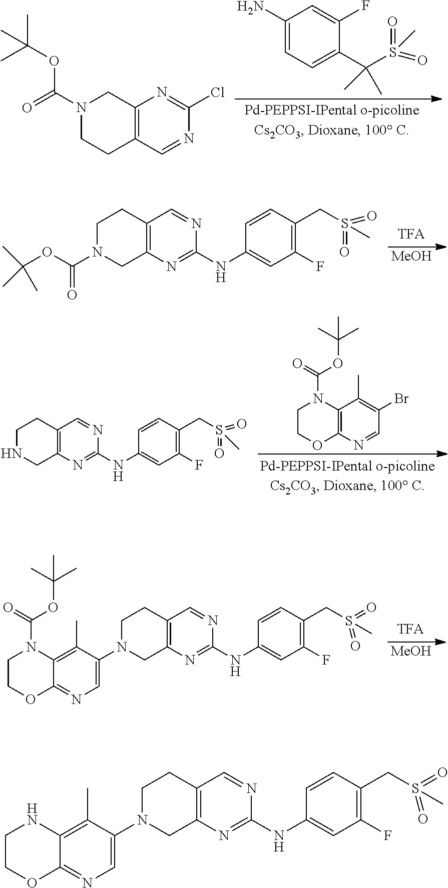

tert-butyl 2-{[3-fluoro-4-(methanesulfonylmethyl)phenyl]amino}-5H,6H,7H,8H-pyrido[3,4-d]pyrimidine-7-carboxylate: In a 40 mL vial were added 3-fluoro-4-(methanesulfonylmethyl)aniline (632.93 mg; 3.11 mmol; 1.20 eq.), tert-butyl 2-chloro-5H,6H,7H,8H-pyrido[3,4-d]pyrimidine-7-carboxylate (700.00 mg; 2.60 mmol; 1.00 eq.), cesium carbonate (1691.15 mg; 5.19 mmol; 2.00 eq.) in dioxane (20 mL) to give a suspension. The brown mixture was degassed with Argon for 5 minutes, then Pd-PEPPSI-IPent Cl 2-methylpyridine (109.15 mg; 0.13 mmol; 0.05 eq.) was added and capped. The mixture was heated at 100° C. for 16 h. LCMS showed the reaction was complete and desired product was observed. The insoluble was removed by filtration. The solvent was concentrated and to get tert-butyl 2-{[3-fluoro-4-(methanesulfonylmethyl)phenyl]amino}-5H,6H,7H,8H-pyrido[3,4-d]pyrimidine-7-carboxylate (850.00 mg; 75% yield) as pale yellow powder, used as is in the next step. MS: m/z 437 (M+H)

N-[3-fluoro-4-(methanesulfonylmethyl)phenyl]-5H,6H,7H,8H-pyrido[3,4-d]pyrimidin-2-amine: To a suspension of tert-butyl 2-{[3-fluoro-4-(methanesulfonylmethyl)phenyl]amino}-5H,6H,7H,8H-pyrido[3,4-d]pyrimidine-7-carboxylate (700.00 mg; 1.60 mmol; 1.00 eq.) in MeOH (1 mL) was added trifluoroacetic acid (9.20 ml; 120.27 mmol; 75.00 eq.). The mixture was stirred at for 15 minutes. LC/MS showed the reaction was complete. After removal of the solvents with Rotavapor, the residue was dissolved in water (10 mL) and adjusted pH to 12-14 with addition of aq NaOH (2N). The precipitated solid was filtered and washed with water three time. The solid was dissolved in DMF and loaded at InterChim column to purify at basic condition (0.1% NH₄OH in water and acetonitrile) to provide N-[3-fluoro-4-(methanesulfonylmethyl)phenyl]-5H,6H,7H,8H-pyrido[3,4-d]pyrimidin-2-amine (450.00 mg; yield in 89%). MS: m/z 337 (M+H).

tert-butyl 7-(2-{[3-fluoro-4-(methanesulfonylmethyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate: To a stirred solution of N-[3-fluoro-4-(methanesulfonylmethyl)phenyl]-5H,6H,7H,8H-pyrido[3,4-d]pyrimidin-2-amine (200.00 mg; 0.59 mmol; 1.00 eq.) and tert-butyl 7-bromo-8-methyl-1H,2H,3H-pyrido[2,3-b][1,4]oxazine-1-carboxylate (226.41 mg; 0.65 mmol; 1.10 eq.) in was added Pd-PEPPSI-IPentCl 2-methylpyridine (o-picoline) (26.29 mg; 0.03 mmol; 0.05 eq.) and dicaesium(1+) carbonate (407.83 mg; 1.19 mmol; 2.00 eq.) in 1,4-dioxane (15 mL). The resulting mixture was stirred for overnight at 100 degrees C. under nitrogen atmosphere. LCMS showed the reaction was complete and desired product was observed. The insoluble was removed by filtration. The solvent was concentrated. The mixture was loaded at InterChim to purified at basic condition (0.1% NH₄OH in water and acetonitrile) get tert-butyl 7-(2-{[3-fluoro-4-(methanesulfonylmethyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (240.00 mg; 69% yield). MS: m/z 585 (M+H)

N-[3-fluoro-4-(methanesulfonylmethyl)phenyl]-7-{8-methyl-1H,2H,3H-pyrido[2,3-b][1,4]oxazin-7-yl}-5H,6H,7H,8H-pyrido[3,4-d]pyrimidin-2-amine: To a suspension of tert-butyl 7-(2-{[3-fluoro-4-(methanesulfonylmethyl)phenyl]amino}-5H,6H,7H,8H-pyrido[3,4-d]pyrimidin-7-yl)-8-methyl-1H,2H,3H-pyrido[2,3-b][1,4]oxazine-1-carboxylate (240.00 mg; 0.41 mmol; 1.00 eq.) in MeOH (1 mL) was added trifluoroacetic acid (2.36 ml; 30.79 mmol; 75.00 eq.). The mixture was stirred at for 15 minutes. LC/MS showed the reaction was complete. After removal of the solvents with Rotavapor, the residue was dissolved in water (10 mL) and adjusted pH to 12-14 with addition of aq NaOH (2N). The precipitated solid was filtered and washed with water three time. The solid was dissolved in DMF and loaded at InterChim column to purify at basic condition (0.1% NH₄OH in water and acetonitrile) to provide N-[3-fluoro-4-(methanesulfonylmethyl) phenyl]-7-{8-methyl-1H,2H,3H-pyrido[2,3-b][1,4]oxazin-7-yl}-5H,6H,7H,8H-pyrido[3,4-d]pyrimidin-2-amine (60.40 mg; yield in 30%). MS: m/z 485 (M+H). ¹H NMR (500 MHz, DMSO) δ 9.93 (s, 1H), 8.41 (s, 1H), 7.87 (d, J=12.9 Hz, 1H), 7.50 (d, J=8.5 Hz, 1H), 7.33 (d, J=8.5 Hz, 1H), 7.30 (d, J=1.9 Hz, 1H), 5.58 (s, 1H), 4.43 (s, 2H), 4.20 (t, J=4.3 Hz, 2H), 4.00 (s, 2H), 3.31 (t, J=4.3 Hz, 2H), 3.13 (t, J=5.9 Hz, 2H), 2.96 (s, 3H), 2.86-2.75 (m, 2H), 2.05 (d, J=1.8 Hz, 3H).

### Example 191: Synthesis of N-{3-[(1H-imidazol-1-yl)methyl]phenyl}-7-{8-methyl-1H,2H,3H-pyrido[2,3-b][1,4]oxazin-7-yl}-5H,6H,7H,8H-pyrido[3,4-d]pyrimidin-2-amine

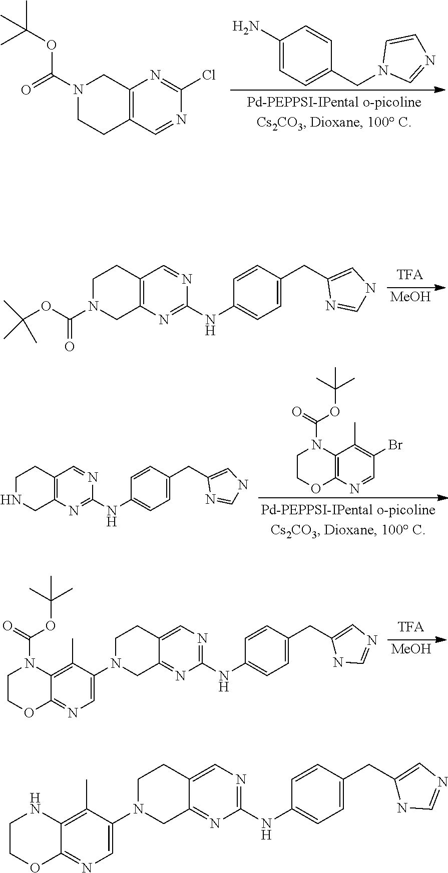

tert-butyl 2-({3-[(1H-imidazol-1-yl)methyl]phenyl}amino)-5H,6H,7H,8H-pyrido[3,4-d]pyrimidine-7-carboxylate: In a 40 mL vial were added 3-[(1H-imidazol-1-yl)methyl]aniline (539.43 mg; 3.11 mmol; 1.20 eq.), tert-butyl 2-chloro-5H,6H,7H,8H-pyrido[3,4-d]pyrimidine-7-carboxylate (700.00 mg; 2.60 mmol; 1.00 eq.), cesium carbonate (1691.15 mg; 5.19 mmol; 2.00 eq.) in dioxane (15 mL) to give a suspension. The brown mixture was degassed with Argon for 5 minutes, then Pd-PEPPSI-IPent Cl 2-methylpyridine (109.15 mg; 0.13 mmol; 0.05 eq.) was added and capped. The mixture was heated at 100° C. for 16 hours. LCMS showed the reaction was complete and desired product was observed. The insoluble was removed by filtration. The solvent was concentrated and to get tert-butyl 2-({3-[(1H-imidazol-1-yl)methyl]phenyl}amino)-5H,6H,7H,8H-pyrido[3,4-d]pyrimidine-7-carboxylate (920.00 mg; 87% yield) as pale yellow powder, used in the next step without further purification. MS: m/z: 407 (M+H).

N-{3-[(1H-imidazol-1-yl)methyl]phenyl}-5H,6H,7H,8H-pyrido[3,4-d]pyrimidin-2-amine: To a suspension of tert-butyl 2-({3-[(1H-imidazol-1-yl)methyl]phenyl}amino)-5H,6H,7H,8H-pyrido[3,4-d]pyrimidine-7-carboxylate (700.00 mg; 1.72 mmol; 1.00 eq.) in MeOH (1 mL) was added trifluoroacetic acid (9.88 ml; 129.16 mmol; 75.00 eq.). The mixture was stirred at RT for 15 minutes. LC/MS showed the reaction was complete. After removal of the solvents with Rotavapor, the residue was dissolved in DMF (6 mL) and adjusted pH to 12-14 with addition of aq NaOH (2N). The mixture was loaded at InterChim column to purify at basic condition (0.1% NH₄OH in water and acetonitrile) to provide N-{3-[(1H-imidazol-1-yl)methyl]phenyl}-5H,6H,7H,8H-pyrido[3,4-d]pyrimidin-2-amine (470.00 mg; 89% yield): MS: m/z 307 (M+H)

tert-butyl 7-[2-({3-[(1H-imidazol-1-yl)methyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate: To a stirred solution of N-{3-[(1H-imidazol-1-yl)methyl]phenyl}-5H,6H,7H,8H-pyrido[3,4-d]pyrimidin-2-amine (200.00 mg; 0.65 mmol; 1.00 eq.) and tert-butyl 7-bromo-8-methyl-1H,2H,3H-pyrido[2,3-b][1,4]oxazine-1-carboxylate (248.60 mg; 0.72 mmol; 1.10 eq.) in was added Pd-PEPPSI-IPentCl 2-methylpyridine (o-picoline) (28.87 mg; 0.03 mmol; 0.05 eq.) and dicaesium(1+) carbonate (447.79 mg; 1.31 mmol; 2.00 eq.) in 1,4-dioxane (15 mL). The resulting mixture was stirred for overnight at 100 degrees C. under nitrogen atmosphere. LCMS showed the reaction was complete and desired product was observed. The insoluble was removed by filtration. The solvent was concentrated. The mixture was loaded at InterChim to purified at basic condition (0.1% NH₄OH in water and acetonitrile) get tert-butyl 7-[2-({3-[(1H-imidazol-1-yl)methyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate (300.00 mg; 83% yield). MS: m/z 555 (M+H).

N-{3-[(1H-imidazol-1-yl)methyl]phenyl}-7-{8-methyl-1H,2H,3H-pyrido[2,3-b][1,4]oxazin-7-yl}-5H,6H,7H,8H-pyrido[3,4-d]pyrimidin-2-amine: To a suspension of tert-butyl 7-[2-({3-[(1H-imidazol-1-yl)methyl]phenyl}amino)-5H,6H,7H,8H-pyrido[3,4-d]pyrimidin-7-yl]-8-methyl-1H,2H,3H-pyrido[2,3-b][1,4]oxazine-1-carboxylate (240.00 mg; 0.43 mmol; 1.00 eq.) in MeOH (1 mL) was added trifluoroacetic acid (2.48 ml; 32.45 mmol; 75.00 eq.). The mixture was stirred at RT for 15 minutes. LC/MS showed the reaction was complete. After removal of the solvents with Rotavapor, the residue was dissolved in water (10 mL) and adjusted pH to 12-14 with addition of aq NaOH (2N). The precipitated solid was filtered and washed with water three time. The solid was dissolved in DMF and loaded at InterChim column to purify at basic condition (0.1% NH4OH in water and acetonitrile) to provide N-{3-[(1H-imidazol-1-yl)methyl]phenyl}-7-{8-methyl-1H,2H,3H-pyrido[2,3-b][1,4]oxazin-7-yl}-5H,6H,7H,8H-pyrido[3,4-d]pyrimidin-2-amine (60.40 mg; 30% yield). MS: m/z 455 (M+H). ¹H NMR (500 MHz, DMSO) δ 9.56 (d, J=2.2 Hz, 1H), 8.32 (d, J=2.2 Hz, 1H), 7.73 (d, J=6.5 Hz, 2H), 7.57 (d, J=8.2 Hz, 1H), 7.30 (d, J=2.1 Hz, 1H), 7.22 (td, J=7.9, 2.2 Hz, 1H), 7.16 (s, 1H), 6.88 (s, 1H), 6.77 (d, J=7.6 Hz, 1H), 5.54 (d, J=2.8 Hz, 1H), 5.16 (d, J=2.2 Hz, 2H), 4.19 (t, J=3.8 Hz, 2H), 3.94 (s, 2H), 3.30 (d, J=5.1 Hz, 2H), 3.11 (d, J=6.0 Hz, 2H), 2.79 (d, J=5.8 Hz, 2H), 2.05 (d, J=2.1 Hz, 3H).

Table 1: Examples with In Vitro and Cellular Activities.

### HPK1 Kinase Assay (Biochemical Assay).

A recombinant fusion protein consisting of full-length human HPK1 (MAP4K1) with an N-terminal Glutatione S-transferase (GST) tag was produced in insect cells Sf21 using the baculovirus expression system. GST-HPK1 protein was purified from cell lysates by glutathione Sepharose affinity chromatography. The assay is run in three continuous steps: 1) the HPK1 enzymatic kinase reaction, 2) an ATP depletion, and 3) the ADP detection, the steps 2 and 3 are performed with ADP-Glo™ Kinase Assay kit from Promega (V9101). Test compounds were prepared by 10-point serial dilution in dimethyl sulfoxide (DMSO) and 100 nL of each dilution was spotted onto a 384-well Optiplate (Perkin Elmer and Cat #6007299) by Labcyte Echo. 5 μL kinase reaction buffer (0.02% Brij-35, 2 mM DTT, 50 mM HEPES pH 7.5, MgCl2 10 mM, BSA 0.01% and O-glycerophosphate 12.5 mM) containing HPK1 (3.2 nM) enzyme was transferred to each well and incubated for 15 minutes at room temperature at 60% humidity. The enzymatic reaction was started by adding 5 μl of Start-Mix (10 μM ATP and 3.235 μM MBP). After 120 minutes, the reaction was stopped by adding 5 μL of ADP-Glo reagent (Promega, V9101) and incubating for 40 min. in the dark at 23° C. To determine the level of ADP, 10 μl ADP-Glo Detection solution was added and incubated for 1 hour at 23° C. in the dark. The plate was transferred to a Perkin Elmer EnVision (2104 Multilabel Reader) for luminescence detection and percent inhibition activity and IC50 value were determined using Genedata Screener.

### Cellular Assay—Phospho-SLP-76 (Ser376).

Inhibition of HPK1 enzymatic activity in cells was determined by detection of SLP76 phosphorylation at Ser376 residue in lysate from Jurkat (human) and EL-4 (mouse) cells. Jurkat and EL-4 cells express both HPK1 and SLP76 naturally. All cells were cultured in RPMI 1640 media containing 10% heat inactivated fetal bovine serum (FBS) and 1×Penicillin-Streptomycin. In either Jurkat or El-4 assay, cells were pre-incubated with test compounds prior to T cell stimulation by respective αCD3/αCD28 Dynabeads™. For detection, plates were read on the Envision and percent inhibition and IC₅₀ were determined by Smart fit in Genedata Analyzer.

For Jurkat cells: 35 μL cell suspension were dispensed at a density of 30,000 cells/well in 384-well plates containing HBSS (Gibco, 14025076)+0.1% BSA). 10-point logarithmic serial dilution of test compounds in DMSO were prepared by a Hamilton automated liquid handling robot and 35 nL was transferred into each well by an Echo liquid handler. After 60 min. pre-incubation, the cells were stimulated by adding 5 μL human T activator αCD3/αCD28 Dynabeads™ (Gibco, 11132D) at 1.2×107 beads/mL for 15 minutes at 37° C. Detection of Ser376 phosphorylation status was accomplished by first adding 10 μL of 5×AlphaLISA lysis buffer (Perkin Elmer, ALSU-PSLP-A500) to each well of the cell plate using a Multidrop Combi (5840300), followed by 20 minutes shaking. 10 μL cell lysate from each treatment were then transferred to a white 384-well plate (Corning 3572) and incubated with AlphaLISA acceptor beads for one hour (AlphaLISA SureFire Ultra p-SLP76(Ser376), Perkin Elmer, ALSU-PSLP-A500), and donor beads for additional 2 hours at room temperature in the dark.

For EL-4.IL2 assay, 50 μL cell suspension at 2×106 cells/mL in HBSS were first dispensed into 384-well plates and then 50 nL serial dilutions of test compounds were spotted to each well by an Echo liquid handler. After 60 min. incubation at 37° C., cells were stimulated by adding 5 μL mouse T cell activator αCD3/αCD28 Dynabeads™ (ThermFisher Scientific, 11452D) for 30 minutes at 37° C. After a brief spin and subsequent supernatant discarded, 12.5 μL of Cisbio lysis buffer were added to each well and plates were shaken for 30 min at 450 RPM. For detection, 4 μL prepared HTRF antibody mixture (Cisbio, 63ADK000U) in detection buffer were mixed with 16 μl of cell lysate and plates were incubated at room temperature for 19 hours prior to EnVision reading.

Results are given in the following table.
For enzymatic activity:
A: IC50<100 nM
B: IC50: 100 nM-1000 nM
C: IC50>1000 nM

For cellular activity:
A: IC50<500 nM
B: IC50: 500 nM-1000 nM
C: IC50>1000 nM

| | | HPK1 | HPK1 |
|---|---|---|---|
| | | enzymatic | pSLP76 |
| # | Structure | IC50 | IC50 |
| Example 1 | 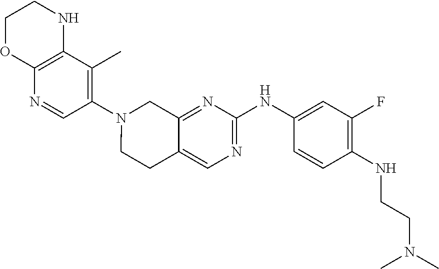 [SMILES: Cc1c(N2CCc3cnc(Nc4ccc(NCCN(C)C)c(F)c4)nc3C2)cnc2c1NCCO2] | A | A |
| Example 2 | 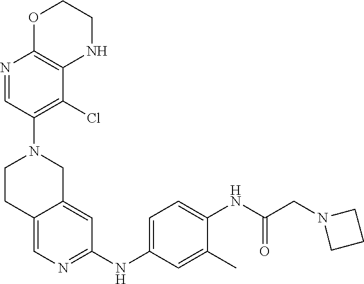 [SMILES: Cc1cc(Nc2cc3c(cn2)CCN(c2cnc4c(c2Cl)NCCO4)C3)ccc1NC(=O)CN1CCC1] | A | A |
| Example 3 | 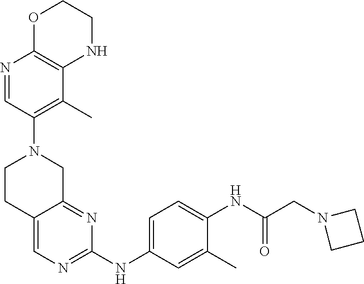 [SMILES: Cc1cc(Nc2ncc3c(n2)CN(c2cnc4c(c2C)NCCO4)CC3)ccc1NC(=O)CN1CCC1] | A | A |
| Example 4 | 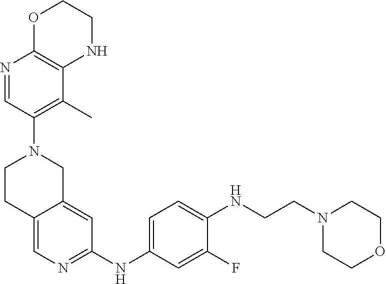 [SMILES: Cc1c(N2CCc3cnc(Nc4ccc(NCCN5CCOCC5)c(F)c4)cc3C2)cnc2c1NCCO2] | A | A |
| Example 5 | 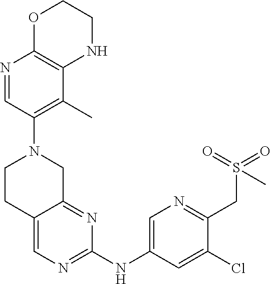 [SMILES: Cc1c(N2CCc3cnc(Nc4cnc(CS(C)(=O)=O)c(Cl)c4)nc3C2)cnc2c1NCCO2] | A | C |
| Example 6 | 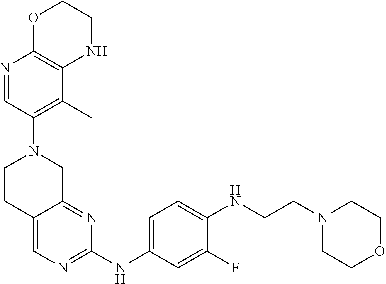 [SMILES: Cc1c(N2CCc3cnc(Nc4ccc(NCCN5CCOCC5)c(F)c4)nc3C2)cnc2c1NCCO2] | A | A |
| Example 7 | 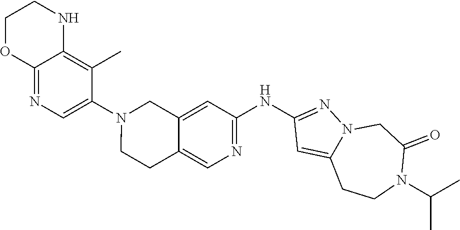 [SMILES: Cc1c(N2CCc3cnc(Nc4cc5n(n4)CC(=O)N(C(C)C)CC5)cc3C2)cnc2c1NCCO2] | A | A |
| Example 8 | 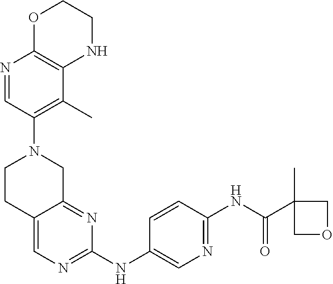 [SMILES: Cc1c(N2CCc3cnc(Nc4ccc(NC(=O)C5(C)COC5)nc4)nc3C2)cnc2c1NCCO2] | B | NA |
| Example 9 | 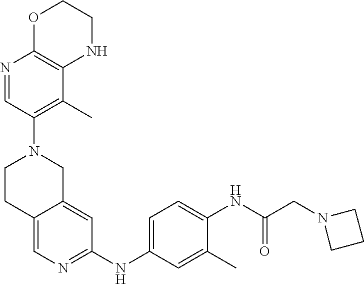 [SMILES: Cc1cc(Nc2cc3c(cn2)CCN(c2cnc4c(c2C)NCCO4)C3)ccc1NC(=O)CN1CCC1] | A | A |
| Example 10 | 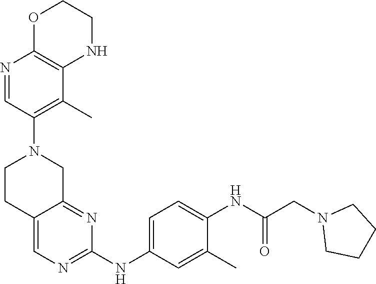 [SMILES: Cc1cc(Nc2ncc3c(n2)CN(c2cnc4c(c2C)NCCO4)CC3)ccc1NC(=O)CN1CCCC1] | A | A |
| Example 11 | 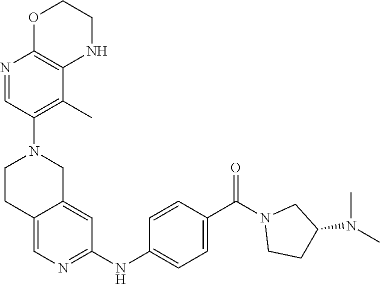 [SMILES: Cc1c(N2CCc3cnc(Nc4ccc(C(=O)N5CC[C@@H](N(C)C)C5)cc4)cc3C2)cnc2c1NCCO2] | A | NA |
| Example 12 | 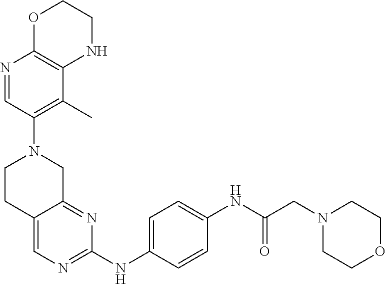 [SMILES: Cc1c(N2CCc3cnc(Nc4ccc(NC(=O)CN5CCOCC5)cc4)nc3C2)cnc2c1NCCO2] | A | A |
| Example 13 | 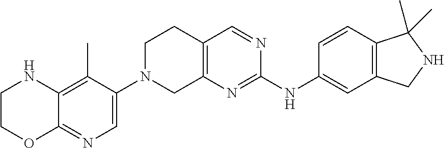 [SMILES: Cc1c(N2CCc3cnc(Nc4ccc5c(c4)CNC5(C)C)nc3C2)cnc2c1NCCO2] | A | A |
| Example 14 | 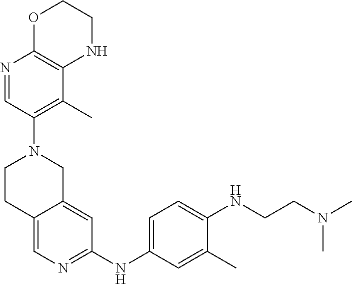 [SMILES: Cc1cc(Nc2cc3c(cn2)CCN(c2cnc4c(c2C)NCCO4)C3)ccc1NCCN(C)C] | A | A |
| Example 15 | 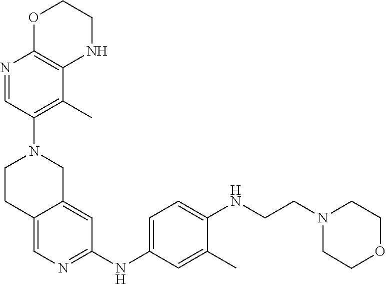 [SMILES: Cc1cc(Nc2cc3c(cn2)CCN(c2cnc4c(c2C)NCCO4)C3)ccc1NCCN1CCOCC1] | A | B |
| Example 16 | 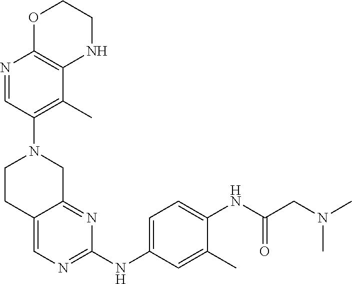 [SMILES: Cc1cc(Nc2ncc3c(n2)CN(c2cnc4c(c2C)NCCO4)CC3)ccc1NC(=O)CN(C)C] | A | A |
| Example 17 | 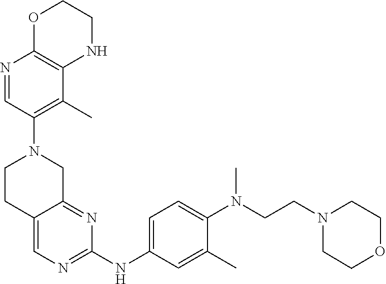 [SMILES: Cc1cc(Nc2ncc3c(n2)CN(c2cnc4c(c2C)NCCO4)CC3)ccc1N(C)CCN1CCOCC1] | A | B |
| Example 18 | 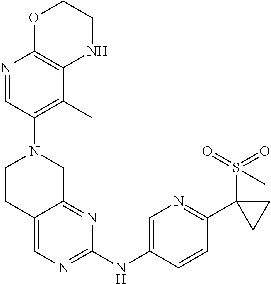 [SMILES: Cc1c(N2CCc3cnc(Nc4ccc(C5(S(C)(=O)=O)CC5)nc4)nc3C2)cnc2c1NCCO2] | A | B |
| Example 19 | 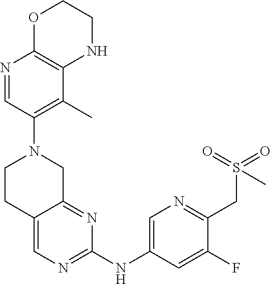 [SMILES: Cc1c(N2CCc3cnc(Nc4cnc(CS(C)(=O)=O)c(F)c4)nc3C2)cnc2c1NCCO2] | A | A |
| Example 20 | 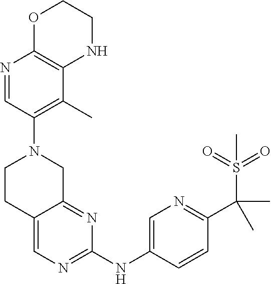 [SMILES: Cc1c(N2CCc3cnc(Nc4ccc(C(C)(C)S(C)(=O)=O)nc4)nc3C2)cnc2c1NCCO2] | A | C |
| Example 21 | 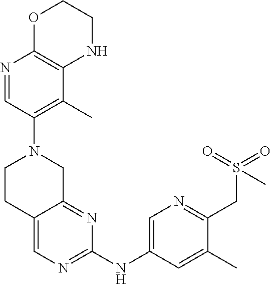 [SMILES: Cc1cc(Nc2ncc3c(n2)CN(c2cnc4c(c2C)NCCO4)CC3)cnc1CS(C)(=O)=O] | A | B |
| Example 22 | 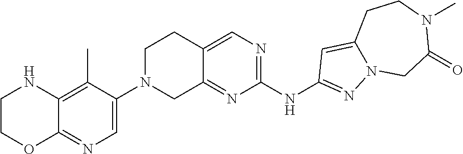 [SMILES: Cc1c(N2CCc3cnc(Nc4cc5n(n4)CC(=O)N(C)CC5)nc3C2)cnc2c1NCCO2] | B | NA |
| Example 23 | 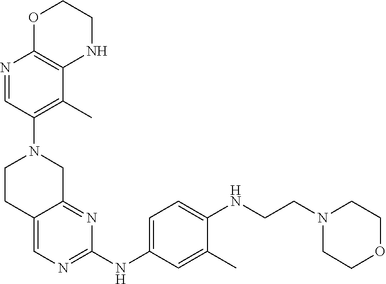 [SMILES: Cc1cc(Nc2ncc3c(n2)CN(c2cnc4c(c2C)NCCO4)CC3)ccc1NCCN1CCOCC1] | A | B |
| Example 24 | 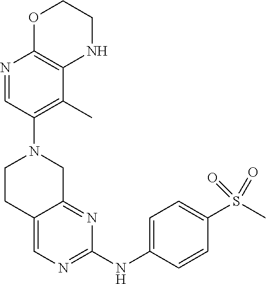 [SMILES: Cc1c(N2CCc3cnc(Nc4ccc(S(C)(=O)=O)cc4)nc3C2)cnc2c1NCCO2] | A | B |
| Example 25 | 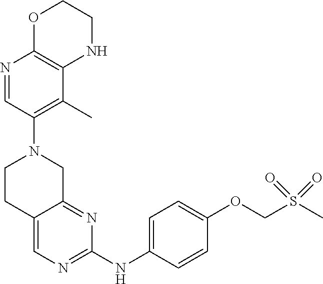 [SMILES: Cc1c(N2CCc3cnc(Nc4ccc(OCS(C)(=O)=O)cc4)nc3C2)cnc2c1NCCO2] | A | A |
| Example 26 | 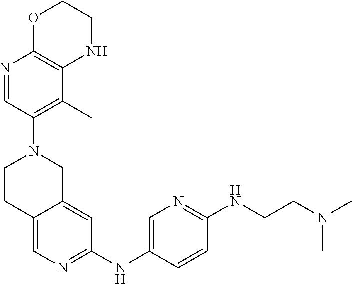 [SMILES: Cc1c(N2CCc3cnc(Nc4ccc(NCCN(C)C)nc4)cc3C2)cnc2c1NCCO2] | A | B |
| Example 27 | 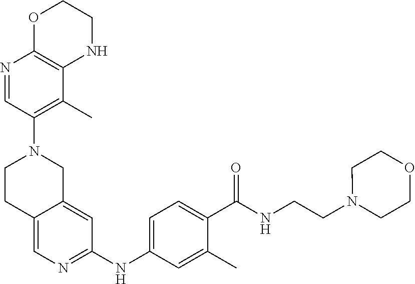 [SMILES: Cc1cc(Nc2cc3c(cn2)CCN(c2cnc4c(c2C)NCCO4)C3)ccc1C(=O)NCCN1CCOCC1] | A | B |
| Example 28 | 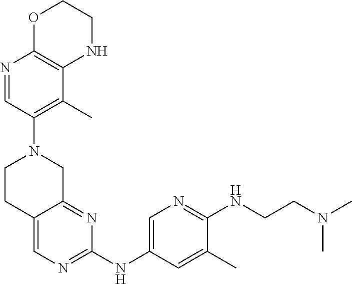 [SMILES: Cc1cc(Nc2ncc3c(n2)CN(c2cnc4c(c2C)NCCO4)CC3)cnc1NCCN(C)C] | A | B |
| Example 29 | 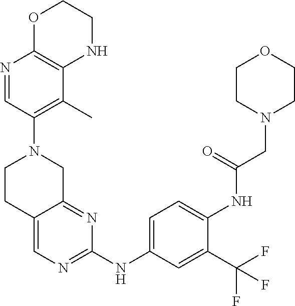 [SMILES: Cc1c(N2CCc3cnc(Nc4ccc(NC(=O)CN5CCOCC5)c(C(F)(F)F)c4)nc3C2)cnc2c1NCCO2] | A | A |
| Example 30 | 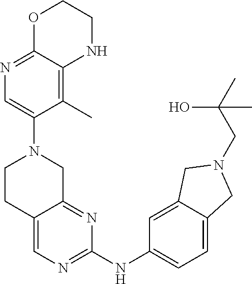 [SMILES: Cc1c(N2CCc3cnc(Nc4ccc5c(c4)CN(CC(C)(C)O)C5)nc3C2)cnc2c1NCCO2] | A | A |
| Example 31 | 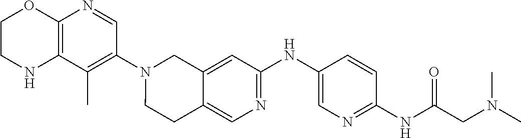 [SMILES: Cc1c(N2CCc3cnc(Nc4ccc(NC(=O)CN(C)C)nc4)cc3C2)cnc2c1NCCO2] | A | C |
| Example 32 | 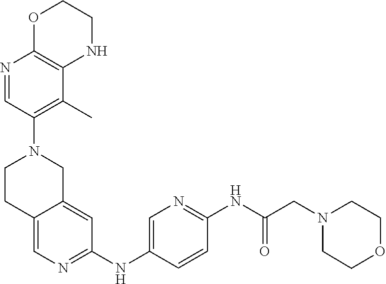 [SMILES: Cc1c(N2CCc3cnc(Nc4ccc(NC(=O)CN5CCOCC5)nc4)cc3C2)cnc2c1NCCO2] | A | C |
| Example 33 | 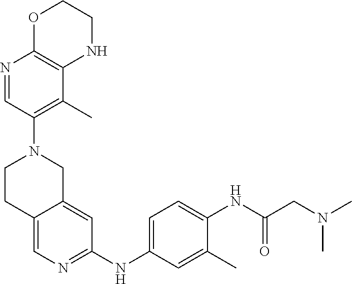 [SMILES: Cc1cc(Nc2cc3c(cn2)CCN(c2cnc4c(c2C)NCCO4)C3)ccc1NC(=O)CN(C)C] | A | C |
| Example 34 | 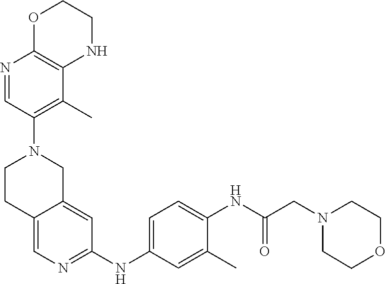 [SMILES: Cc1cc(Nc2cc3c(cn2)CCN(c2cnc4c(c2C)NCCO4)C3)ccc1NC(=O)CN1CCOCC1] | A | A |
| Example 35 | 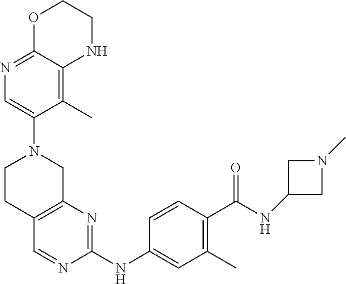 [SMILES: Cc1cc(Nc2ncc3c(n2)CN(c2cnc4c(c2C)NCCO4)CC3)ccc1C(=O)NC1CN(C)C1] | A | B |
| Example 36 | 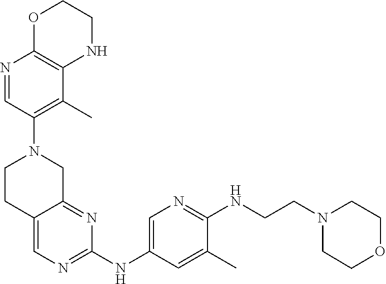 [SMILES: Cc1cc(Nc2ncc3c(n2)CN(c2cnc4c(c2C)NCCO4)CC3)cnc1NCCN1CCOCC1] | A | B |
| Example 37 | 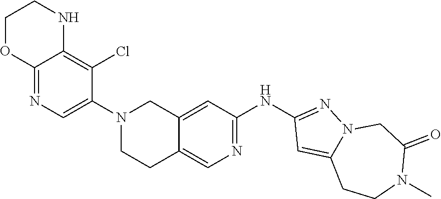 [SMILES: CN1CCc2cc(Nc3cc4c(cn3)CCN(c3cnc5c(c3Cl)NCCO5)C4)nn2CC1=O] | A | B |
| Example 38 | 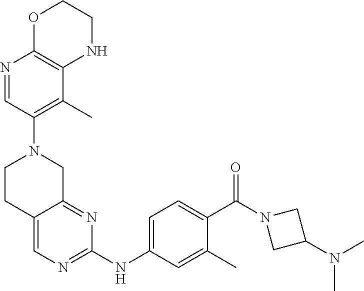 [SMILES: Cc1cc(Nc2ncc3c(n2)CN(c2cnc4c(c2C)NCCO4)CC3)ccc1C(=O)N1CC(N(C)C)C1] | A | C |
| Example 39 | 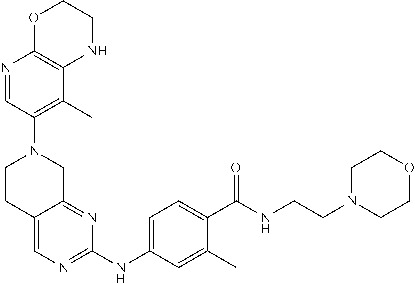 [SMILES: Cc1cc(Nc2ncc3c(n2)CN(c2cnc4c(c2C)NCCO4)CC3)ccc1C(=O)NCCN1CCOCC1] | A | A |
| Example 40 | 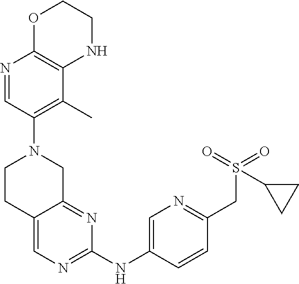 [SMILES: Cc1c(N2CCc3cnc(Nc4ccc(CS(=O)(=O)C5CC5)nc4)nc3C2)cnc2c1NCCO2] | A | A |
| Example 41 | 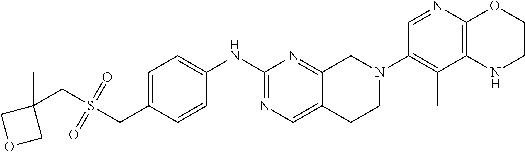 [SMILES: Cc1c(N2CCc3cnc(Nc4ccc(CS(=O)(=O)CC5(C)COC5)cc4)nc3C2)cnc2c1NCCO2] | A | A |
| Example 42 | 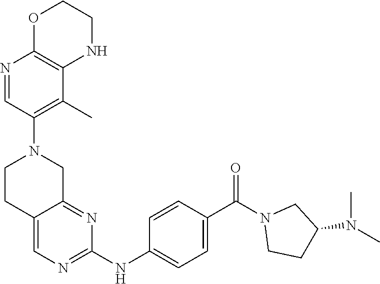 [SMILES: Cc1c(N2CCc3cnc(Nc4ccc(C(=O)N5CC[C@@H](N(C)C)C5)cc4)nc3C2)cnc2c1NCCO2] | A | A |
| Example 43 | 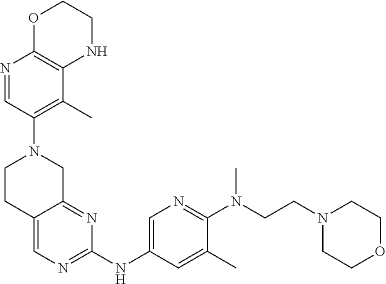 [SMILES: Cc1cc(Nc2ncc3c(n2)CN(c2cnc4c(c2C)NCCO4)CC3)cnc1N(C)CCN1CCOCC1] | A | NA |
| Example 44 | 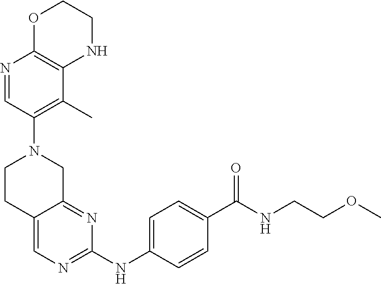 [SMILES: COCCNC(=O)c1ccc(Nc2ncc3c(n2)CN(c2cnc4c(c2C)NCCO4)CC3)cc1] | A | A |
| Example 45 | 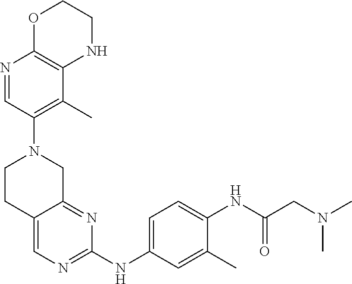 [SMILES: Cc1cc(Nc2ncc3c(n2)CN(c2cnc4c(c2C)NCCO4)CC3)ccc1NC(=O)CN(C)C] | A | A |
| Example 46 | 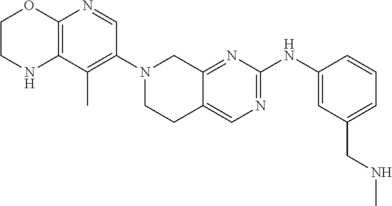 [SMILES: CNCc1cccc(Nc2ncc3c(n2)CN(c2cnc4c(c2C)NCCO4)CC3)c1] | A | B |
| Example 47 | 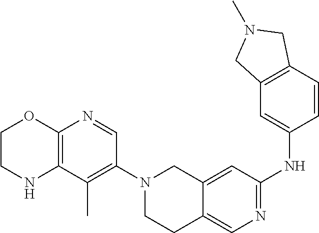 [SMILES: Cc1c(N2CCc3cnc(Nc4ccc5c(c4)CN(C)C5)cc3C2)cnc2c1NCCO2] | A | B |
| Eaxmple 48 | 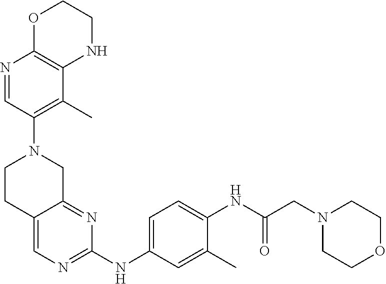 [SMILES: Cc1cc(Nc2ncc3c(n2)CN(c2cnc4c(c2C)NCCO4)CC3)ccc1NC(=O)CN1CCOCC1] | A | A |
| Example 49 | 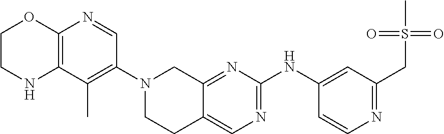 [SMILES: Cc1c(N2CCc3cnc(Nc4ccnc(CS(C)(=O)=O)c4)nc3C2)cnc2c1NCCO2] | A | B |
| Example 50 | 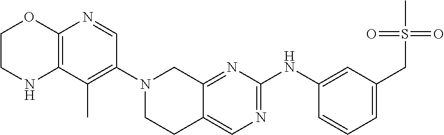 [SMILES: Cc1c(N2CCc3cnc(Nc4cccc(CS(C)(=O)=O)c4)nc3C2)cnc2c1NCCO2] | A | A |
| Example 51 | 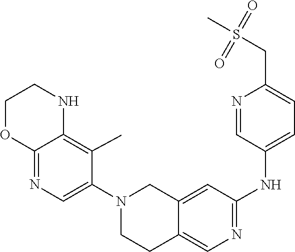 [SMILES: Cc1c(N2CCc3cnc(Nc4ccc(CS(C)(=O)=O)nc4)cc3C2)cnc2c1NCCO2] | A | B |
| Example 52 | 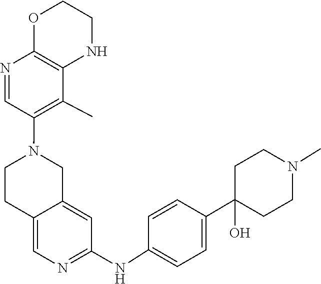 [SMILES: Cc1c(N2CCc3cnc(Nc4ccc(C5(O)CCN(C)CC5)cc4)cc3C2)cnc2c1NCCO2] | A | B |
| Example 53 | 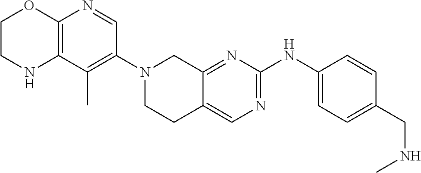 [SMILES: CNCc1ccc(Nc2ncc3c(n2)CN(c2cnc4c(c2C)NCCO4)CC3)cc1] | A | B |
| Example 54 | 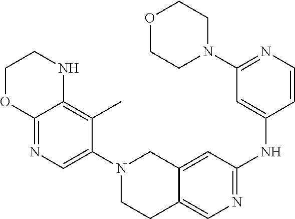 [SMILES: Cc1c(N2CCc3cnc(Nc4ccnc(N5CCOCC5)c4)cc3C2)cnc2c1NCCO2] | A | NA |
| Example 55 | 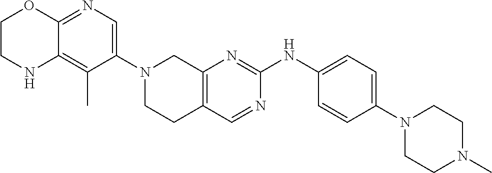 [SMILES: Cc1c(N2CCc3cnc(Nc4ccc(N5CCN(C)CC5)cc4)nc3C2)cnc2c1NCCO2] | A | B |
| Example 56 | 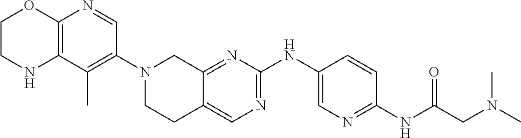 [SMILES: Cc1c(N2CCc3cnc(Nc4ccc(NC(=O)CN(C)C)nc4)nc3C2)cnc2c1NCCO2] | A | A |
| Example 57 | 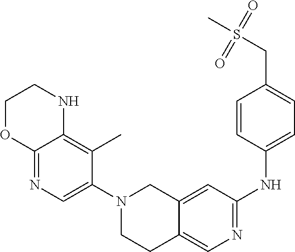 [SMILES: Cc1c(N2CCc3cnc(Nc4ccc(CS(C)(=O)=O)cc4)cc3C2)cnc2c1NCCO2] | A | A |
| Example 58 | 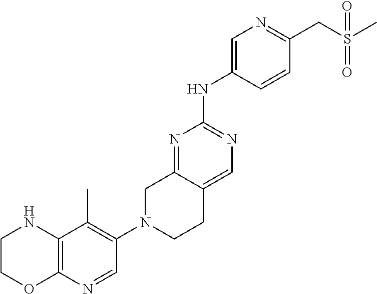 [SMILES: Cc1c(N2CCc3cnc(Nc4ccc(CS(C)(=O)=O)nc4)nc3C2)cnc2c1NCCO2] | A | A |
| Example 59 | 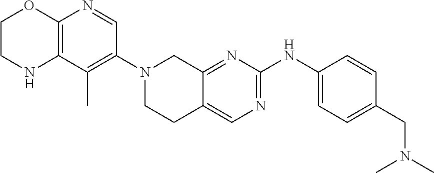 [SMILES: Cc1c(N2CCc3cnc(Nc4ccc(CN(C)C)cc4)nc3C2)cnc2c1NCCO2] | A | C |
| Example 60 | 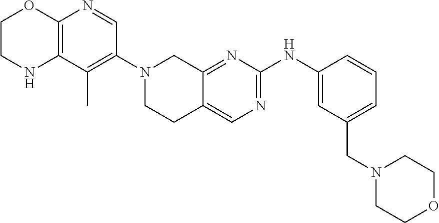 [SMILES: Cc1c(N2CCc3cnc(Nc4cccc(CN5CCOCC5)c4)nc3C2)cnc2c1NCCO2] | A | B |
| Example 61 | 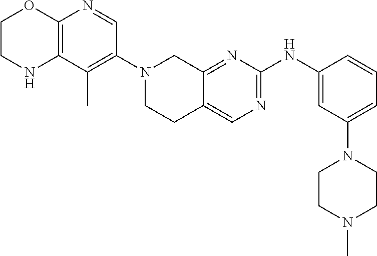 [SMILES: Cc1c(N2CCc3cnc(Nc4cccc(N5CCN(C)CC5)c4)nc3C2)cnc2c1NCCO2] | A | B |
| Example 62 | 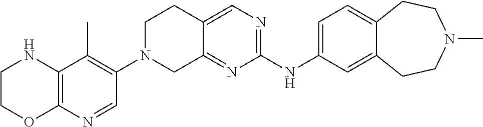 [SMILES: Cc1c(N2CCc3cnc(Nc4ccc5c(c4)CCN(C)CC5)nc3C2)cnc2c1NCCO2] | A | NA |
| Example 63 | 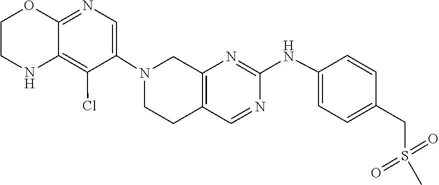 [SMILES: CS(=O)(=O)Cc1ccc(Nc2ncc3c(n2)CN(c2cnc4c(c2Cl)NCCO4)CC3)cc1] | A | A |
| Example 64 | 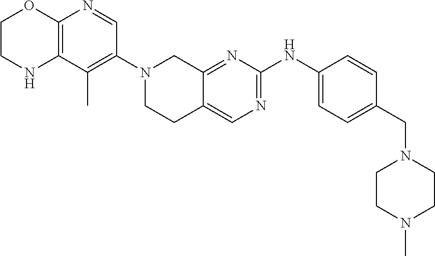 [SMILES: Cc1c(N2CCc3cnc(Nc4ccc(CN5CCN(C)CC5)cc4)nc3C2)cnc2c1NCCO2] | A | NA |
| Example 65 | 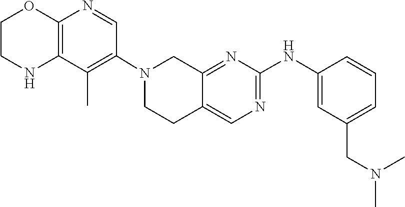 [SMILES: Cc1c(N2CCc3cnc(Nc4cccc(CN(C)C)c4)nc3C2)cnc2c1NCCO2] | A | B |
| Example 66 | 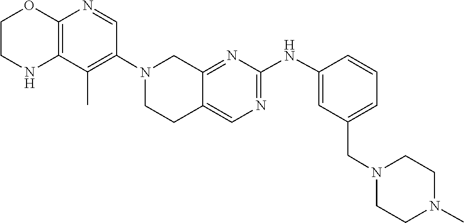 [SMILES: Cc1c(N2CCc3cnc(Nc4cccc(CN5CCN(C)CC5)c4)nc3C2)cnc2c1NCCO2] | A | B |
| Example 67 | 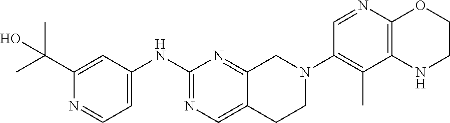 [SMILES: Cc1c(N2CCc3cnc(Nc4ccnc(C(C)(C)O)c4)nc3C2)cnc2c1NCCO2] | A | B |
| Example 68 | 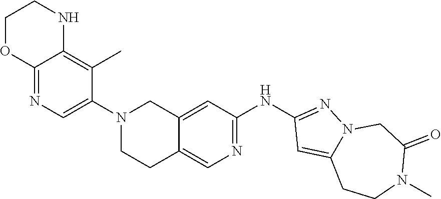 [SMILES: Cc1c(N2CCc3cnc(Nc4cc5n(n4)CC(=O)N(C)CC5)cc3C2)cnc2c1NCCO2] | A | A |
| Example 69 | 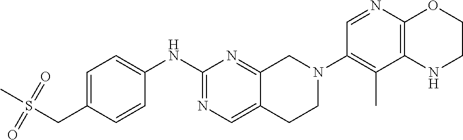 [SMILES: Cc1c(N2CCc3cnc(Nc4ccc(CS(C)(=O)=O)cc4)nc3C2)cnc2c1NCCO2] | A | A |
| Example 70 | 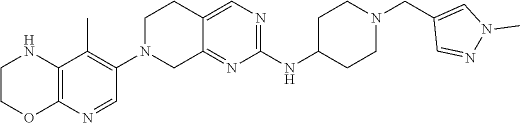 [SMILES: Cc1c(N2CCc3cnc(NC4CCN(Cc5cnn(C)c5)CC4)nc3C2)cnc2c1NCCO2] | C | NA |
| Example 71 | 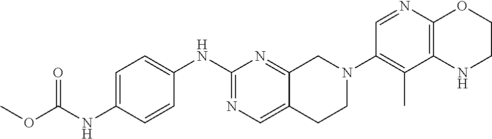 [SMILES: COC(=O)Nc1ccc(Nc2ncc3c(n2)CN(c2cnc4c(c2C)NCCO4)CC3)cc1] | A | A |
| Example 72 | 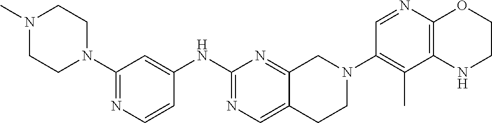 [SMILES: Cc1c(N2CCc3cnc(Nc4ccnc(N5CCN(C)CC5)c4)nc3C2)cnc2c1NCCO2] | A | A |
| Example 73 | 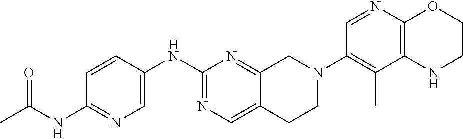 [SMILES: CC(=O)Nc1ccc(Nc2ncc3c(n2)CN(c2cnc4c(c2C)NCCO4)CC3)cn1] | A | B |
| Example 74 | 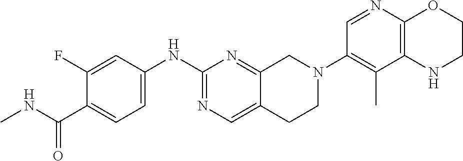 [SMILES: CNC(=O)c1ccc(Nc2ncc3c(n2)CN(c2cnc4c(c2C)NCCO4)CC3)cc1F] | A | A |
| Example 75 | 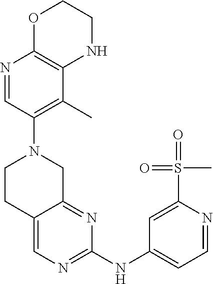 [SMILES: Cc1c(N2CCc3cnc(Nc4ccnc(S(C)(=O)=O)c4)nc3C2)cnc2c1NCCO2] | A | NA |
| Example 76 | 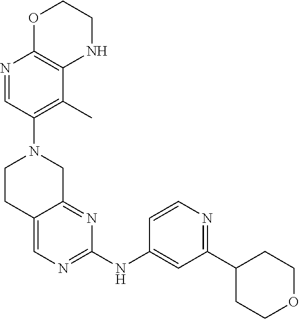 [SMILES: Cc1c(N2CCc3cnc(Nc4ccnc(C5CCOCC5)c4)nc3C2)cnc2c1NCCO2] | A | A |
| Example 77 | 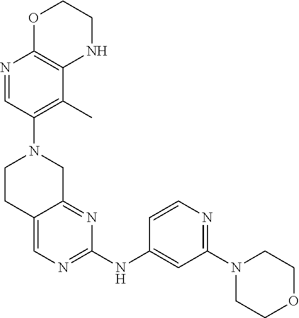 [SMILES: Cc1c(N2CCc3cnc(Nc4ccnc(N5CCOCC5)c4)nc3C2)cnc2c1NCCO2] | A | A |
| Example 78 | 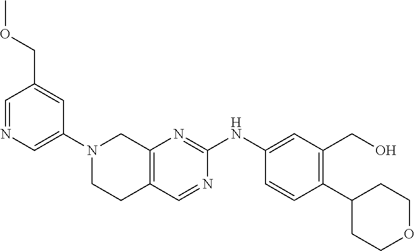 [SMILES: COCc1cncc(N2CCc3cnc(Nc4ccc(C5CCOCC5)c(CO)c4)nc3C2)c1] | B | NA |
| Example 79 | 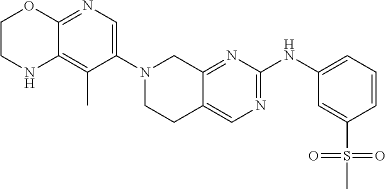 [SMILES: Cc1c(N2CCc3cnc(Nc4cccc(S(C)(=O)=O)c4)nc3C2)cnc2c1NCCO2] | A | A |
| Example 80 | 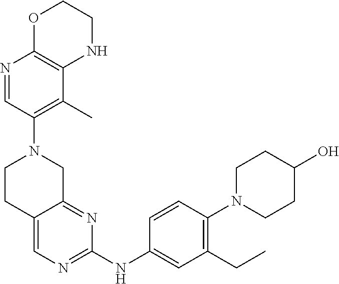 [SMILES: CCc1cc(Nc2ncc3c(n2)CN(c2cnc4c(c2C)NCCO4)CC3)ccc1N1CCC(O)CC1] | A | B |
| Example 81 | 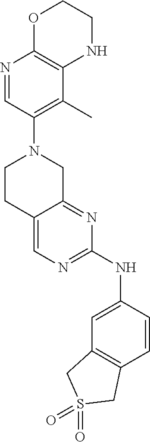 [SMILES: Cc1c(N2CCc3cnc(Nc4ccc5c(c4)CS(=O)(=O)C5)nc3C2)cnc2c1NCCO2] | A | A |
| Example 82 | 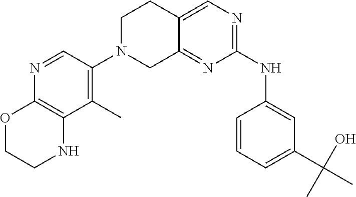 [SMILES: Cc1c(N2CCc3cnc(Nc4cccc(C(C)(C)O)c4)nc3C2)cnc2c1NCCO2] | A | A |
| Example 83 | 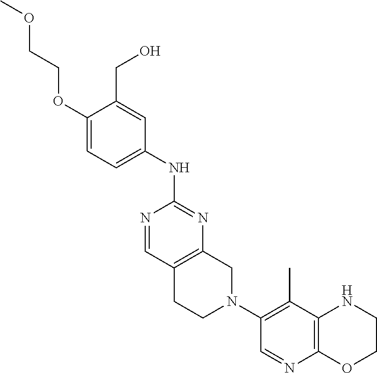 [SMILES: COCCOc1ccc(Nc2ncc3c(n2)CN(c2cnc4c(c2C)NCCO4)CC3)cc1CO] | A | B |
| Example 84 | 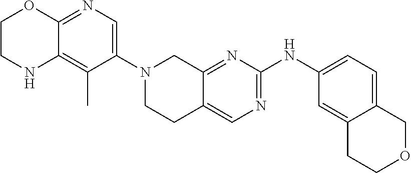 [SMILES: Cc1c(N2CCc3cnc(Nc4ccc5c(c4)CCOC5)nc3C2)cnc2c1NCCO2] | A | A |
| Example 85 | 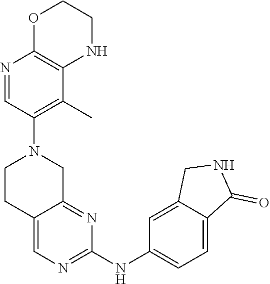 [SMILES: Cc1c(N2CCc3cnc(Nc4ccc5c(c4)CNC5=O)nc3C2)cnc2c1NCCO2] | A | C |
| Example 86 | 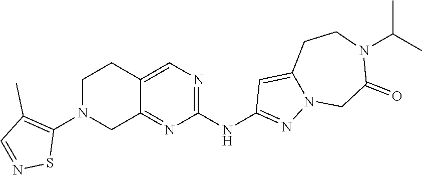 [SMILES: Cc1cnsc1N1CCc2cnc(Nc3cc4n(n3)CC(=O)N(C(C)C)CC4)nc2C1] | C | NA |
| Example 87 | 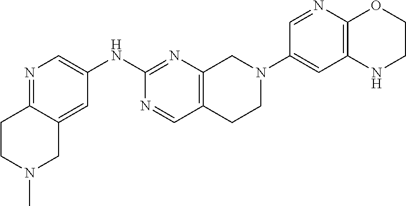 [SMILES: CN1CCc2ncc(Nc3ncc4c(n3)CN(c3cnc5c(c3)NCCO5)CC4)cc2C1] | B | C |
| Example 88 | 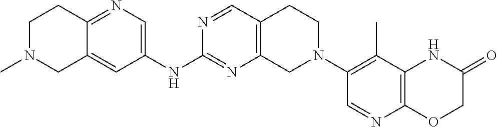 [SMILES: Cc1c(N2CCc3cnc(Nc4cnc5c(c4)CN(C)CC5)nc3C2)cnc2c1NC(=O)CO2] | A | NA |
| Example 89 | 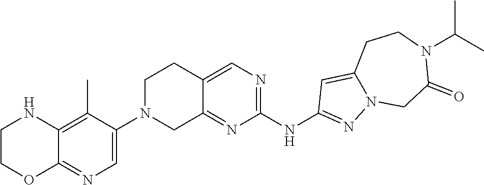 [SMILES: Cc1c(N2CCc3cnc(Nc4cc5n(n4)CC(=O)N(C(C)C)CC5)nc3C2)cnc2c1NCCO2] | C | NA |
| Example 90 | 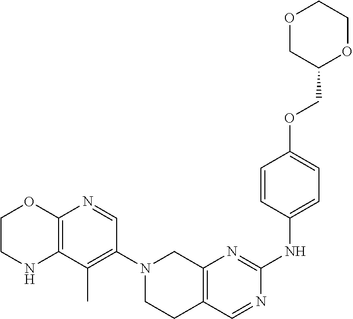 [SMILES: Cc1c(N2CCc3cnc(Nc4ccc(OC[C@@H]5COCCO5)cc4)nc3C2)cnc2c1NCCO2] | A | B |
| Example 91 | 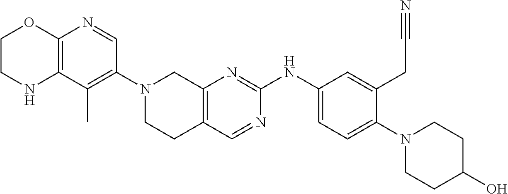 [SMILES: Cc1c(N2CCc3cnc(Nc4ccc(N5CCC(O)CC5)c(CC#N)c4)nc3C2)cnc2c1NCCO2] | A | A |
| Example 92 | 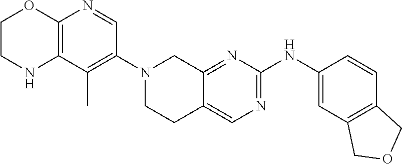 [SMILES: Cc1c(N2CCc3cnc(Nc4ccc5c(c4)COC5)nc3C2)cnc2c1NCCO2] | A | B |
| Example 93 | 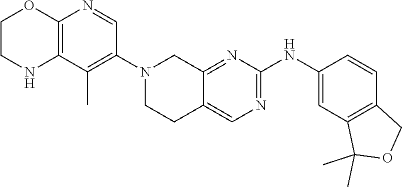 [SMILES: Cc1c(N2CCc3cnc(Nc4ccc5c(c4)C(C)(C)OC5)nc3C2)cnc2c1NCCO2] | A | A |
| Example 94 | 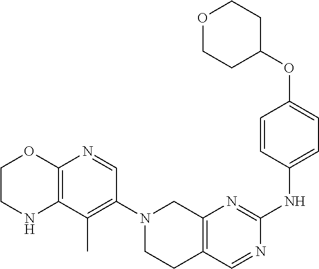 [SMILES: Cc1c(N2CCc3cnc(Nc4ccc(OC5CCOCC5)cc4)nc3C2)cnc2c1NCCO2] | A | B |
| Example 95 | 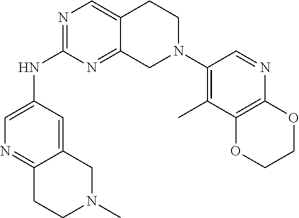 [SMILES: Cc1c(N2CCc3cnc(Nc4cnc5c(c4)CN(C)CC5)nc3C2)cnc2c1OCCO2] | A | NA |
| Example 96 | 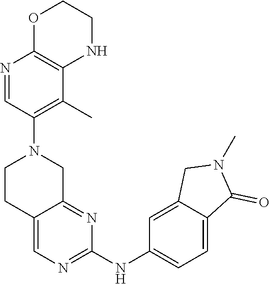 [SMILES: Cc1c(N2CCc3cnc(Nc4ccc5c(c4)CN(C)C5=O)nc3C2)cnc2c1NCCO2] | A | C |
| Example 97 | 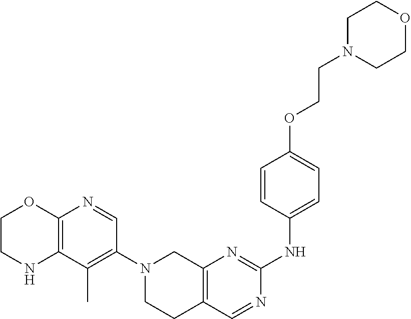 [SMILES: Cc1c(N2CCc3cnc(Nc4ccc(OCCN5CCOCC5)cc4)nc3C2)cnc2c1NCCO2] | A | B |
| Example 98 | 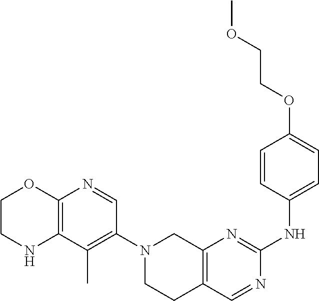 [SMILES: COCCOc1ccc(Nc2ncc3c(n2)CN(c2cnc4c(c2C)NCCO4)CC3)cc1] | A | B |
| Example 99 | 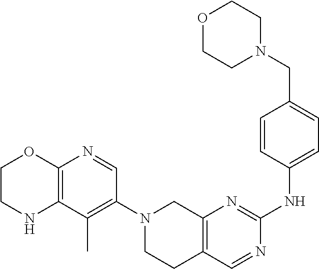 [SMILES: Cc1c(N2CCc3cnc(Nc4ccc(CN5CCOCC5)cc4)nc3C2)cnc2c1NCCO2] | A | B |
| Example 100 | 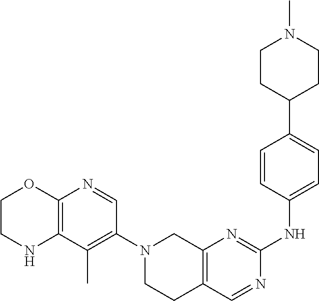 [SMILES: Cc1c(N2CCc3cnc(Nc4ccc(C5CCN(C)CC5)cc4)nc3C2)cnc2c1NCCO2] | A | B |
| Example 101 | 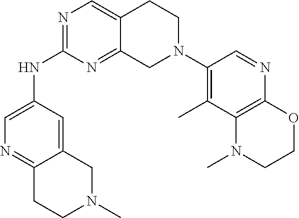 [SMILES: Cc1c(N2CCc3cnc(Nc4cnc5c(c4)CN(C)CC5)nc3C2)cnc2c1N(C)CCO2] | A | NA |
| Example 102 | 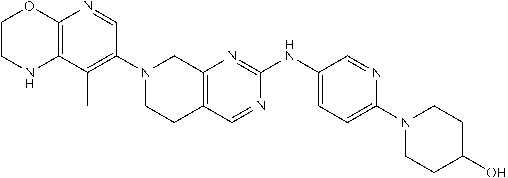 [SMILES: Cc1c(N2CCc3cnc(Nc4ccc(N5CCC(O)CC5)nc4)nc3C2)cnc2c1NCCO2] | A | C |
| Example 103 | 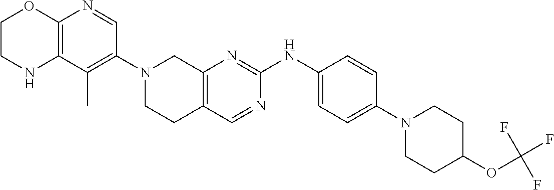 [SMILES: Cc1c(N2CCc3cnc(Nc4ccc(N5CCC(OC(F)(F)F)CC5)cc4)nc3C2)cnc2c1NCCO2] | A | C |
| Example 104 | 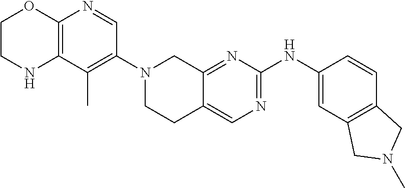 [SMILES: Cc1c(N2CCc3cnc(Nc4ccc5c(c4)CN(C)C5)nc3C2)cnc2c1NCCO2] | A | A |
| Example 105 | 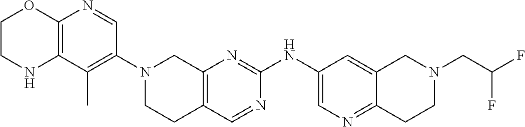 [SMILES: Cc1c(N2CCc3cnc(Nc4cnc5c(c4)CN(CC(F)F)CC5)nc3C2)cnc2c1NCCO2] | A | B |
| Example 106 | 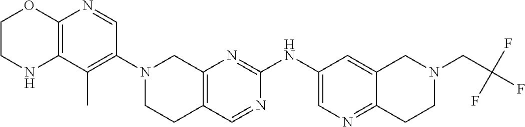 [SMILES: Cc1c(N2CCc3cnc(Nc4cnc5c(c4)CN(CC(F)(F)F)CC5)nc3C2)cnc2c1NCCO2] | A | C |
| Example 107 | 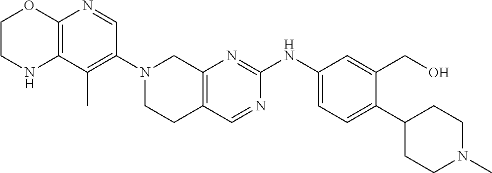 [SMILES: Cc1c(N2CCc3cnc(Nc4ccc(C5CCN(C)CC5)c(CO)c4)nc3C2)cnc2c1NCCO2] | A | B |
| Example 108 | 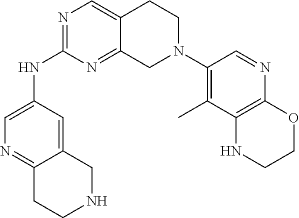 [SMILES: Cc1c(N2CCc3cnc(Nc4cnc5c(c4)CNCC5)nc3C2)cnc2c1NCCO2] | A | B |
| Example 109 | 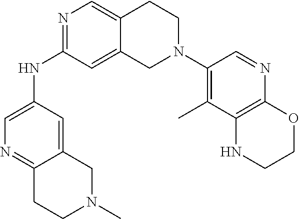 [SMILES: Cc1c(N2CCc3cnc(Nc4cnc5c(c4)CN(C)CC5)cc3C2)cnc2c1NCCO2] | B | C |
| Example 110 | 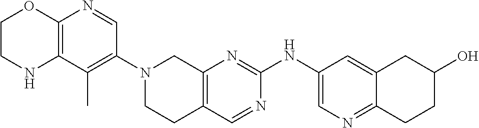 [SMILES: Cc1c(N2CCc3cnc(Nc4cnc5c(c4)CC(O)CC5)nc3C2)cnc2c1NCCO2] | A | B |
| Example 111 | 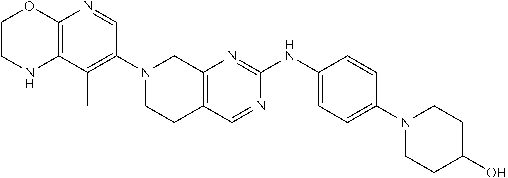 [SMILES: Cc1c(N2CCc3cnc(Nc4ccc(N5CCC(O)CC5)cc4)nc3C2)cnc2c1NCCO2] | A | B |
| Example 112 | 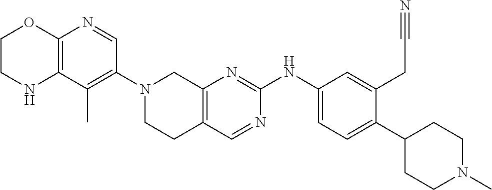 [SMILES: Cc1c(N2CCc3cnc(Nc4ccc(C5CCN(C)CC5)c(CC#N)c4)nc3C2)cnc2c1NCCO2] | A | B |
| Example 113 | 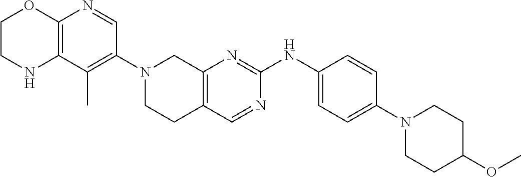 [SMILES: COC1CCN(c2ccc(Nc3ncc4c(n3)CN(c3cnc5c(c3C)NCCO5)CC4)cc2)CC1] | A | B |
| Example 114 | 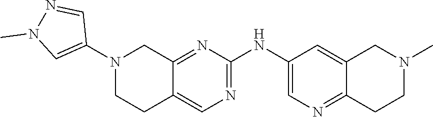 | A | NA |
| Example 115 | 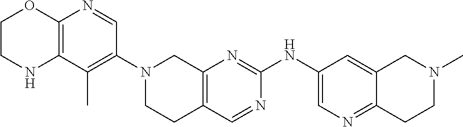 [SMILES: Cc1c(N2CCc3cnc(Nc4cnc5c(c4)CN(C)CC5)nc3C2)cnc2c1NCCO2] | A | B |
| Example 116 | 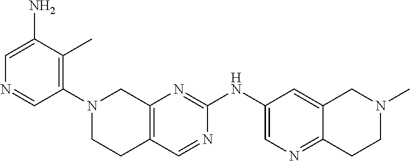 [SMILES: Cc1c(N)cncc1N1CCc2cnc(Nc3cnc4c(c3)CN(C)CC4)nc2C1] | A | C |
| Example 117 | 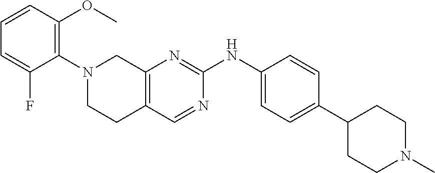 [SMILES: COc1cccc(F)c1N1CCc2cnc(Nc3ccc(C4CCN(C)CC4)cc3)nc2C1] | A | NA |
| Example 118 | 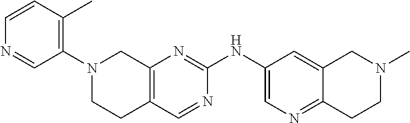 [SMILES: Cc1ccncc1N1CCc2cnc(Nc3cnc4c(c3)CN(C)CC4)nc2C1] | A | C |
| Example 119 | 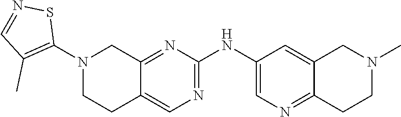 [SMILES: Cc1cnsc1N1CCc2cnc(Nc3cnc4c(c3)CN(C)CC4)nc2C1] | A | C |
| Example 120 | 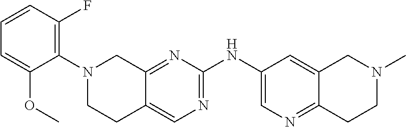 [SMILES: COc1cccc(F)c1N1CCc2cnc(Nc3cnc4c(c3)CN(C)CC4)nc2C1] | A | C |
| Example 121 | 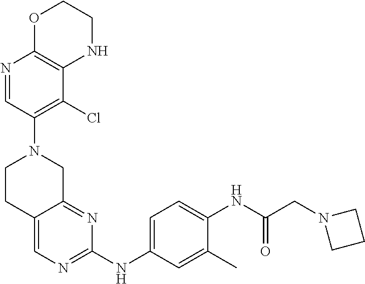 [SMILES: Cc1cc(Nc2ncc3c(n2)CN(c2cnc4c(c2Cl)NCCO4)CC3)ccc1NC(=O)CN1CCC1] | A | A |
| Example 122 | 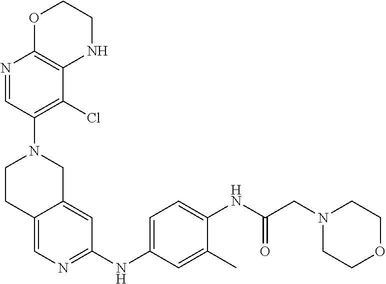 [SMILES: Cc1cc(Nc2cc3c(cn2)CCN(c2cnc4c(c2Cl)NCCO4)C3)ccc1NC(=O)CN1CCOCC1] | A | B |
| Example 123 | 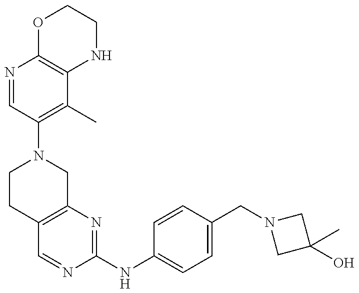 [SMILES: Cc1c(N2CCc3cnc(Nc4ccc(CN5CC(C)(O)C5)cc4)nc3C2)cnc2c1NCCO2] | B | NA |
| Example 124 | 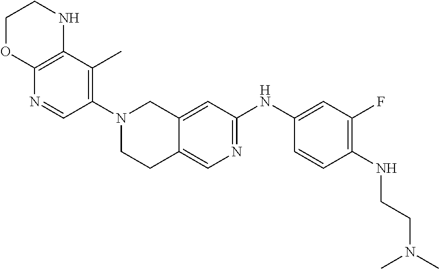 [SMILES: Cc1c(N2CCc3cnc(Nc4ccc(NCCN(C)C)c(F)c4)cc3C2)cnc2c1NCCO2] | A | B |
| Example 125 | 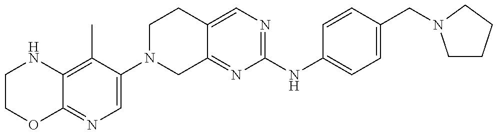 [SMILES: Cc1c(N2CCc3cnc(Nc4ccc(CN5CCCC5)cc4)nc3C2)cnc2c1NCCO2] | A | C |
| Example 126 | 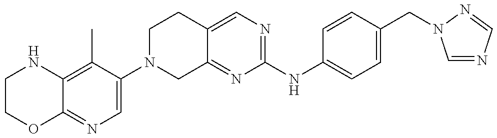 [SMILES: Cc1c(N2CCc3cnc(Nc4ccc(Cn5cncn5)cc4)nc3C2)cnc2c1NCCO2] | A | A |
| Example 127 | 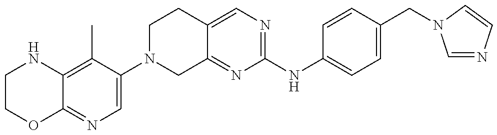 [SMILES: Cc1c(N2CCc3cnc(Nc4ccc(Cn5ccnc5)cc4)nc3C2)cnc2c1NCCO2] | A | A |
| Example 128 | 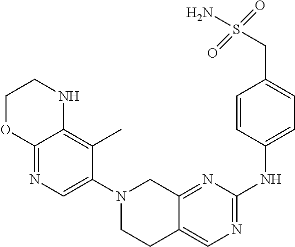 [SMILES: Cc1c(N2CCc3cnc(Nc4ccc(CS(N)(=O)=O)cc4)nc3C2)cnc2c1NCCO2] | A | A |
| Example 129 | 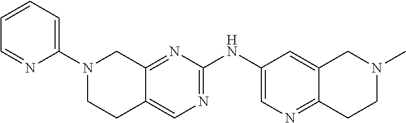 [SMILES: CN1CCc2ncc(Nc3ncc4c(n3)CN(c3ccccn3)CC4)cc2C1] | B | NA |
| Example 130 | 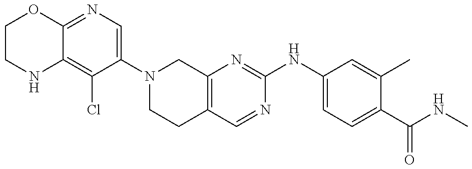 [SMILES: CNC(=O)c1ccc(Nc2ncc3c(n2)CN(c2cnc4c(c2Cl)NCCO4)CC3)cc1C] | A | B |
| Example 131 | 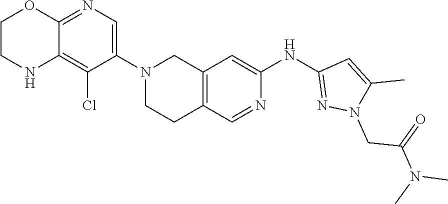 [SMILES: Cc1cc(Nc2cc3c(cn2)CCN(c2cnc4c(c2Cl)NCCO4)C3)nn1CC(=O)N(C)C] | C | NA |
| Example 132 | 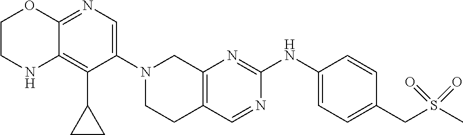 [SMILES: CS(=O)(=O)Cc1ccc(Nc2ncc3c(n2)CN(c2cnc4c(c2C2CC2)NCCO4)CC3)cc1] | A | A |
| Example 133 | 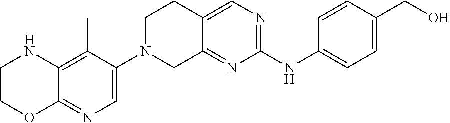 [SMILES: Cc1c(N2CCc3cnc(Nc4ccc(CO)cc4)nc3C2)cnc2c1NCCO2] | A | A |
| Example 134 | 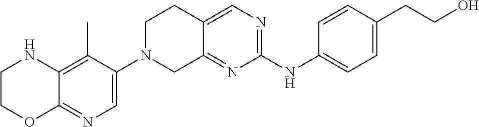 [SMILES: Cc1c(N2CCc3cnc(Nc4ccc(CCO)cc4)nc3C2)cnc2c1NCCO2] | A | NA |
| Example 135 | 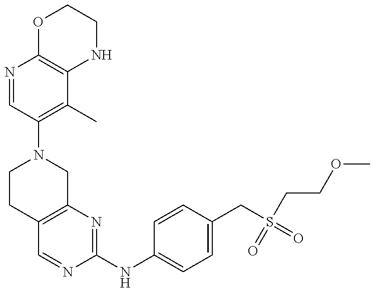 [SMILES: COCCS(=O)(=O)Cc1ccc(Nc2ncc3c(n2)CN(c2cnc4c(c2C)NCCO4)CC3)cc1] | A | A |
| Example 136 | 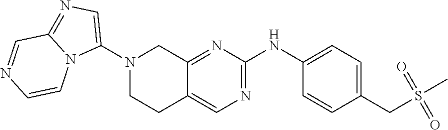 [SMILES: CS(=O)(=O)Cc1ccc(Nc2ncc3c(n2)CN(c2cnc4cnccn24)CC3)cc1] | C | NA |
| Example 137 | 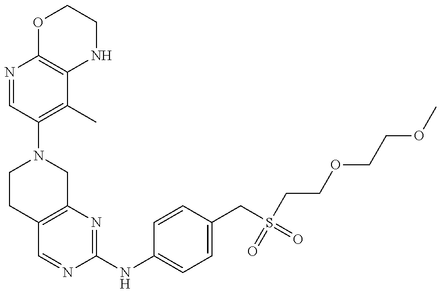 [SMILES: COCCOCCS(=O)(=O)Cc1ccc(Nc2ncc3c(n2)CN(c2cnc4c(c2C)NCCO4)CC3)cc1] | A | NA |
| Example 138 | 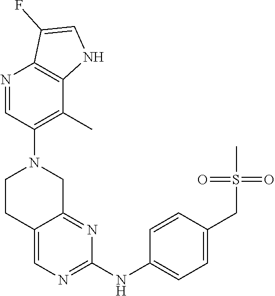 [SMILES: Cc1c(N2CCc3cnc(Nc4ccc(CS(C)(=O)=O)cc4)nc3C2)cnc2c(F)c[nH]c12] | A | A |
| Example 139 | 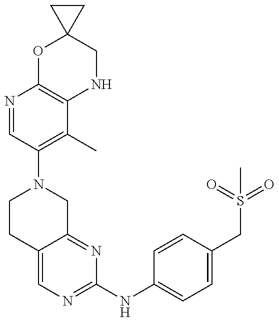 [SMILES: Cc1c(N2CCc3cnc(Nc4ccc(CS(C)(=O)=O)cc4)nc3C2)cnc2c1NCC1(CC1)O2] | A | A |
| Example 140 | 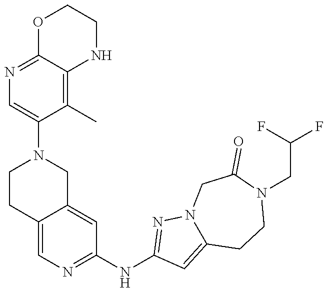 [SMILES: Cc1c(N2CCc3cnc(Nc4cc5n(n4)CC(=O)N(CC(F)F)CC5)cc3C2)cnc2c1NCCO2] | A | A |
| Example 141 | 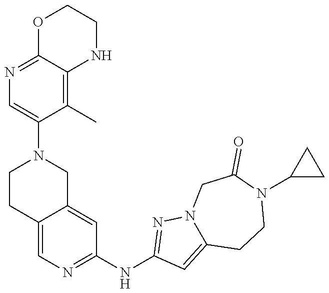 [SMILES: Cc1c(N2CCc3cnc(Nc4cc5n(n4)CC(=O)N(C4CC4)CC5)cc3C2)cnc2c1NCCO2] | A | NA |
| Example 142 | 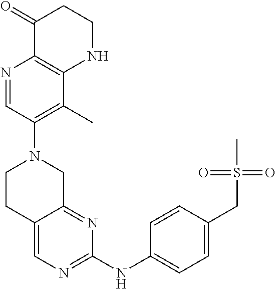 [SMILES: Cc1c(N2CCc3cnc(Nc4ccc(CS(C)(=O)=O)cc4)nc3C2)cnc2c1NCCC2=O] | A | C |
| Example 143 | 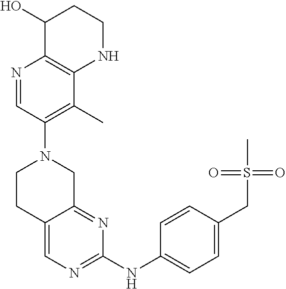 [SMILES: Cc1c(N2CCc3cnc(Nc4ccc(CS(C)(=O)=O)cc4)nc3C2)cnc2c1NCCC2O] | A | A |
| Example 144 | 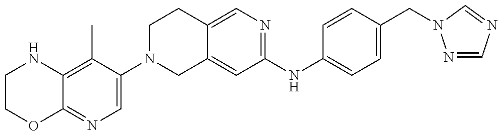 [SMILES: Cc1c(N2CCc3cnc(Nc4ccc(Cn5cncn5)cc4)cc3C2)cnc2c1NCCO2] | A | A |
| Example 145 | 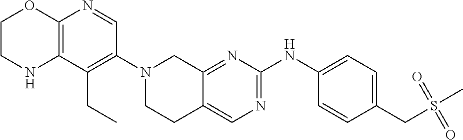 [SMILES: CCc1c(N2CCc3cnc(Nc4ccc(CS(C)(=O)=O)cc4)nc3C2)cnc2c1NCCO2] | A | A |
| Example 146 | 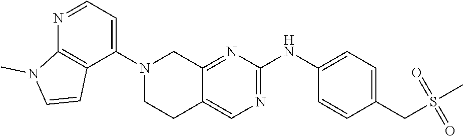 | C | NA |
| Example 147 | 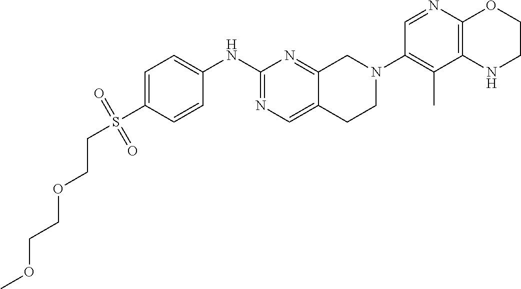 [SMILES: COCCOCCS(=O)(=O)c1ccc(Nc2ncc3c(n2)CN(c2cnc4c(c2C)NCCO4)CC3)cc1] | A | A |
| Example 148 | 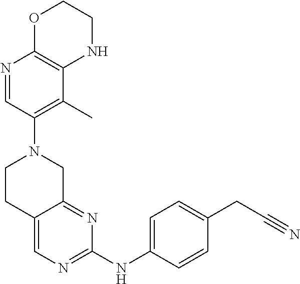 [SMILES: Cc1c(N2CCc3cnc(Nc4ccc(CC#N)cc4)nc3C2)cnc2c1NCCO2] | A | A |
| Example 149 | 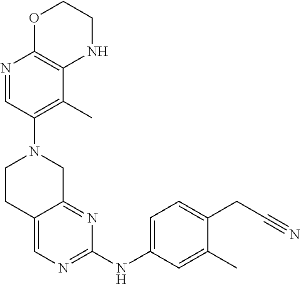 [SMILES: Cc1cc(Nc2ncc3c(n2)CN(c2cnc4c(c2C)NCCO4)CC3)ccc1CC#N] | A | NA |
| Example 150 | 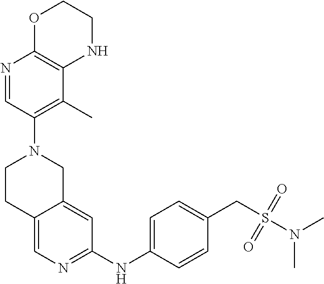 [SMILES: Cc1c(N2CCc3cnc(Nc4ccc(CS(=O)(=O)N(C)C)cc4)cc3C2)cnc2c1NCCO2] | A | A |
| Example 151 | 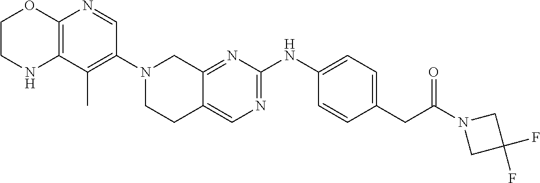 [SMILES: Cc1c(N2CCc3cnc(Nc4ccc(CC(=O)N5CC(F)(F)C5)cc4)nc3C2)cnc2c1NCCO2] | A | A |
| Example 152 | 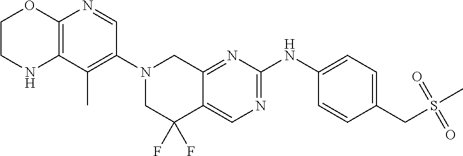 [SMILES: Cc1c(N2Cc3nc(Nc4ccc(CS(C)(=O)=O)cc4)ncc3C(F)(F)C2)cnc2c1NCCO2] | A | A |
| Example 153 | 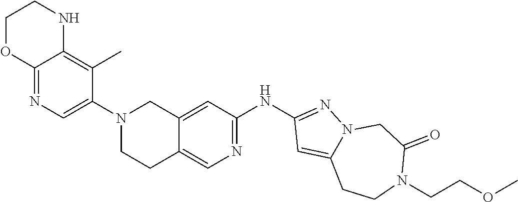 [SMILES: COCCN1CCc2cc(Nc3cc4c(cn3)CCN(c3cnc5c(c3C)NCCO5)C4)nn2CC1=O] | A | A |
| Example 154 | 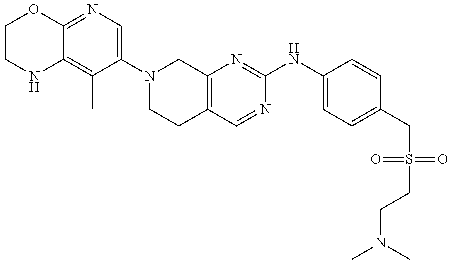 [SMILES: Cc1c(N2CCc3cnc(Nc4ccc(CS(=O)(=O)CCN(C)C)cc4)nc3C2)cnc2c1NCCO2] | A | A |
| Example 155 | 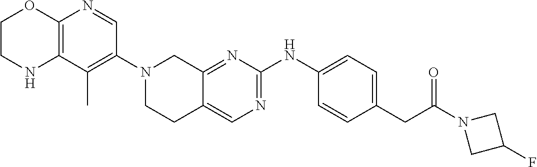 [SMILES: Cc1c(N2CCc3cnc(Nc4ccc(CC(=O)N5CC(F)C5)cc4)nc3C2)cnc2c1NCCO2] | A | A |
| Example 156 | 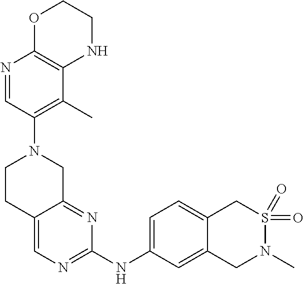 [SMILES: Cc1c(N2CCc3cnc(Nc4ccc5c(c4)CN(C)S(=O)(=O)C5)nc3C2)cnc2c1NCCO2] | A | A |
| Example 157 | 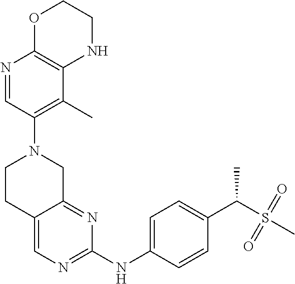 [SMILES: Cc1c(N2CCc3cnc(Nc4ccc([C@H](C)S(C)(=O)=O)cc4)nc3C2)cnc2c1NCCO2] | A | A |
| Example 158 | 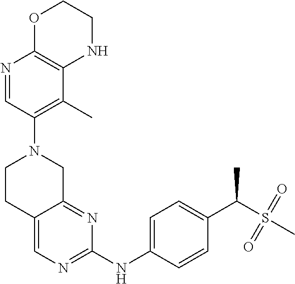 [SMILES: Cc1c(N2CCc3cnc(Nc4ccc([C@@H](C)S(C)(=O)=O)cc4)nc3C2)cnc2c1NCCO2] | A | A |
| Example 159 | 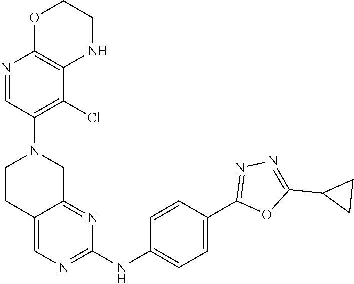 [SMILES: Clc1c(N2CCc3cnc(Nc4ccc(-c5nnc(C6CC6)o5)cc4)nc3C2)cnc2c1NCCO2] | A | NA |
| Example 160 | 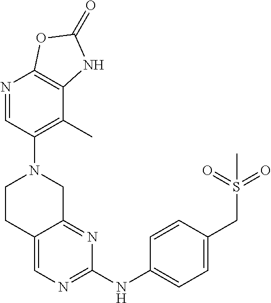 [SMILES: Cc1c(N2CCc3cnc(Nc4ccc(CS(C)(=O)=O)cc4)nc3C2)cnc2oc(=O)[nH]c12] | A | NA |
| Example 161 | 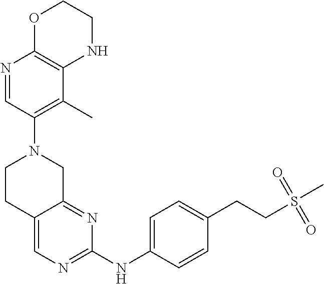 [SMILES: Cc1c(N2CCc3cnc(Nc4ccc(CCS(C)(=O)=O)cc4)nc3C2)cnc2c1NCCO2] | A | A |
| Example 162 | 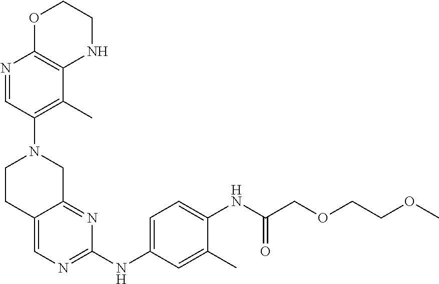 [SMILES: COCCOCC(=O)Nc1ccc(Nc2ncc3c(n2)CN(c2cnc4c(c2C)NCCO4)CC3)cc1C] | A | A |
| Example 163 | 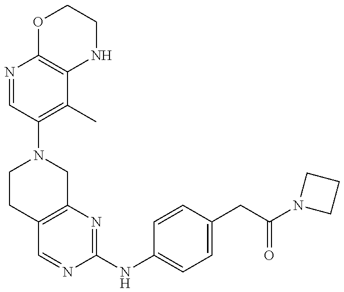 [SMILES: Cc1c(N2CCc3cnc(Nc4ccc(CC(=O)N5CCC5)cc4)nc3C2)cnc2c1NCCO2] | A | A |
| Example 164 | 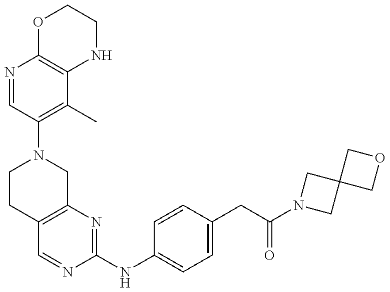 [SMILES: Cc1c(N2CCc3cnc(Nc4ccc(CC(=O)N5CC6(COC6)C5)cc4)nc3C2)cnc2c1NCCO2] | A | A |
| Example 165 | 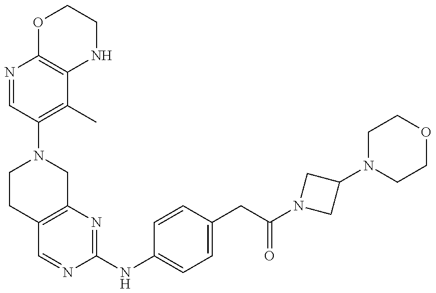 [SMILES: Cc1c(N2CCc3cnc(Nc4ccc(CC(=O)N5CC(N6CCOCC6)C5)cc4)nc3C2)cnc2c1NCCO2] | A | A |
| Example 168 | 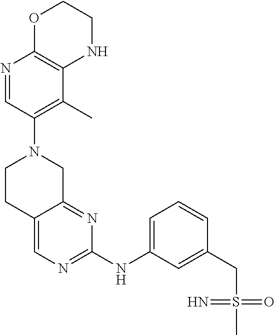 [SMILES: Cc1c(N2CCc3cnc(Nc4cccc(CS(C)(=N)=O)c4)nc3C2)cnc2c1NCCO2] | A | A |
| Example 169 | 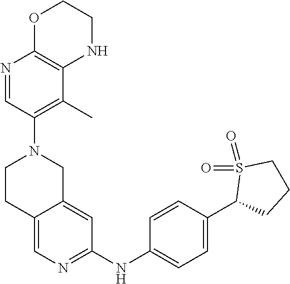 [SMILES: Cc1c(N2CCc3cnc(Nc4ccc([C@H]5CCCS5(=O)=O)cc4)cc3C2)cnc2c1NCCO2] | A | A |
| Example 170 | 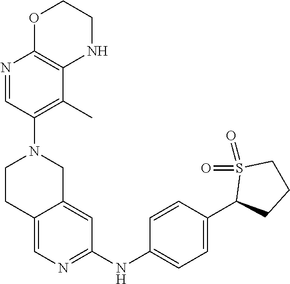 [SMILES: Cc1c(N2CCc3cnc(Nc4ccc([C@@H]5CCCS5(=O)=O)cc4)cc3C2)cnc2c1NCCO2] | A | A |
| Example 171 | 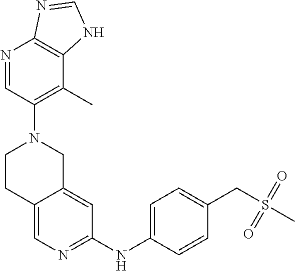 [SMILES: Cc1c(N2CCc3cnc(Nc4ccc(CS(C)(=O)=O)cc4)cc3C2)cnc2nc[nH]c12] | A | B |
| Example 172 | 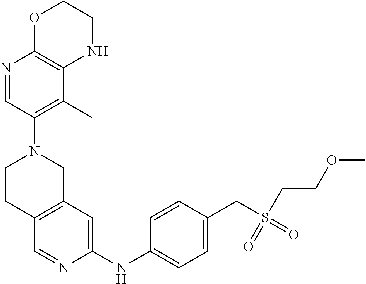 [SMILES: COCCS(=O)(=O)Cc1ccc(Nc2cc3c(cn2)CCN(c2cnc4c(c2C)NCCO4)C3)cc1] | A | NA |
| Example 173 | 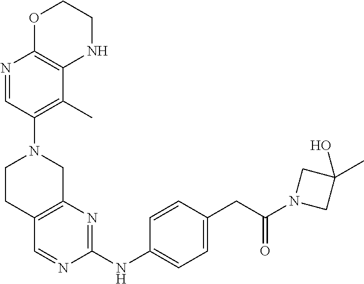 [SMILES: Cc1c(N2CCc3cnc(Nc4ccc(CC(=O)N5CC(C)(O)C5)cc4)nc3C2)cnc2c1NCCO2] | A | A |
| Example 174 | 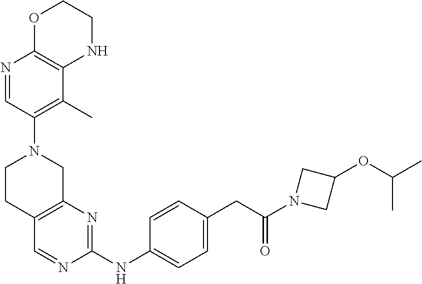 [SMILES: Cc1c(N2CCc3cnc(Nc4ccc(CC(=O)N5CC(OC(C)C)C5)cc4)nc3C2)cnc2c1NCCO2] | A | A |
| Example 175 | 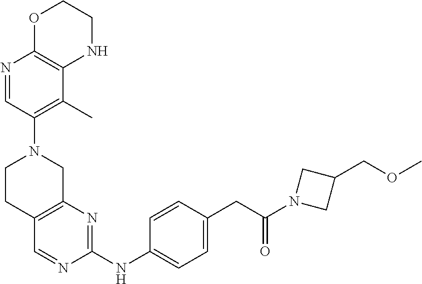 [SMILES: COCC1CN(C(=O)Cc2ccc(Nc3ncc4c(n3)CN(c3cnc5c(c3C)NCCO5)CC4)cc2)C1] | A | A |
| Example 176 | 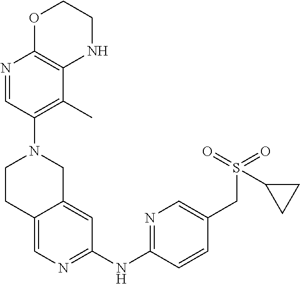 [SMILES: Cc1c(N2CCc3cnc(Nc4ccc(CS(=O)(=O)C5CC5)cn4)cc3C2)cnc2c1NCCO2] | A | A |
| Example 177 | 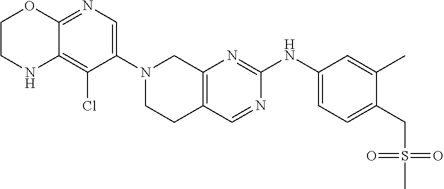 [SMILES: Cc1cc(Nc2ncc3c(n2)CN(c2cnc4c(c2Cl)NCCO4)CC3)ccc1CS(C)(=O)=O] | A | NA |
| Example 178 | 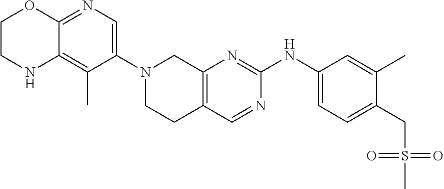 [SMILES: Cc1cc(Nc2ncc3c(n2)CN(c2cnc4c(c2C)NCCO4)CC3)ccc1CS(C)(=O)=O] | A | A |
| Example 179 | 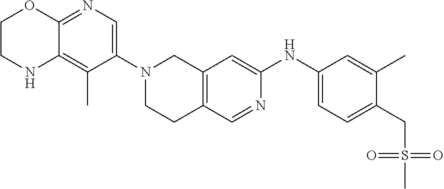 [SMILES: Cc1cc(Nc2cc3c(cn2)CCN(c2cnc4c(c2C)NCCO4)C3)ccc1CS(C)(=O)=O] | A | A |
| Example 180 | 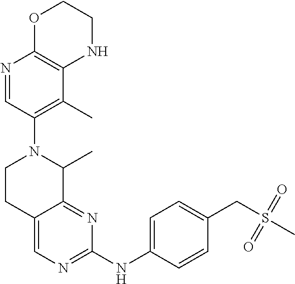 [SMILES: Cc1c(N2CCc3cnc(Nc4ccc(CS(C)(=O)=O)cc4)nc3C2C)cnc2c1NCCO2] | A | A |
| Example 181 | 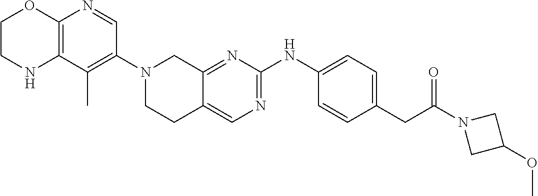 [SMILES: COC1CN(C(=O)Cc2ccc(Nc3ncc4c(n3)CN(c3cnc5c(c3C)NCCO5)CC4)cc2)C1] | A | A |
| Example 182 | 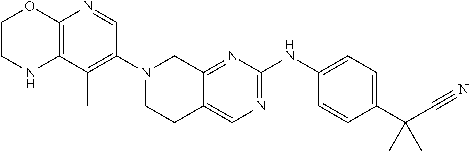 [SMILES: Cc1c(N2CCc3cnc(Nc4ccc(C(C)(C)C#N)cc4)nc3C2)cnc2c1NCCO2] | A | A |
| Example 183 | 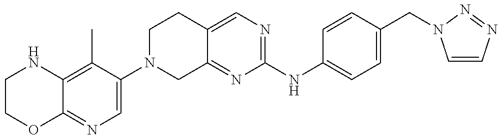 [SMILES: Cc1c(N2CCc3cnc(Nc4ccc(Cn5ccnn5)cc4)nc3C2)cnc2c1NCCO2] | A | A |
| Example 185 | 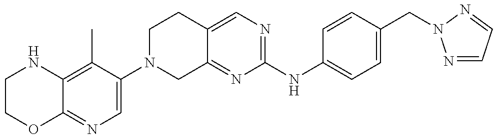 [SMILES: Cc1c(N2CCc3cnc(Nc4ccc(Cn5nccn5)cc4)nc3C2)cnc2c1NCCO2] | A | A |
| Example 186 | 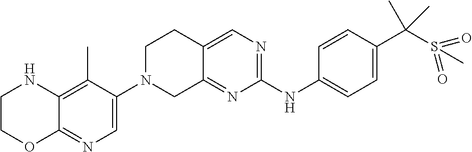 [SMILES: Cc1c(N2CCc3cnc(Nc4ccc(C(C)(C)S(C)(=O)=O)cc4)nc3C2)cnc2c1NCCO2] | A | B |
| Example 187 | 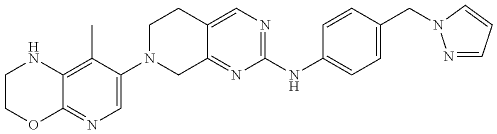 [SMILES: Cc1c(N2CCc3cnc(Nc4ccc(Cn5cccn5)cc4)nc3C2)cnc2c1NCCO2] | A | A |
| Example 188 | 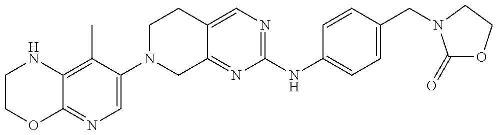 [SMILES: Cc1c(N2CCc3cnc(Nc4ccc(CN5CCOC5=O)cc4)nc3C2)cnc2c1NCCO2] | A | A |
| Example 189 | 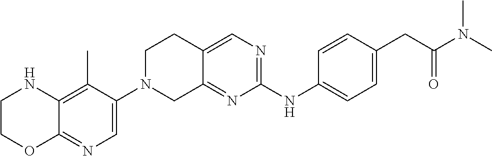 [SMILES: Cc1c(N2CCc3cnc(Nc4ccc(CC(=O)N(C)C)cc4)nc3C2)cnc2c1NCCO2] | A | A |
| Example 190 | 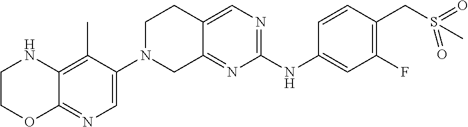 [SMILES: Cc1c(N2CCc3cnc(Nc4ccc(CS(C)(=O)=O)c(F)c4)nc3C2)cnc2c1NCCO2] | A | A |
| Example 191 | 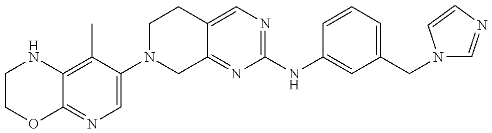 [SMILES: Cc1c(N2CCc3cnc(Nc4cccc(Cn5ccnc5)c4)nc3C2)cnc2c1NCCO2] | A | A |

What is claimed:

1. A compound of formula I 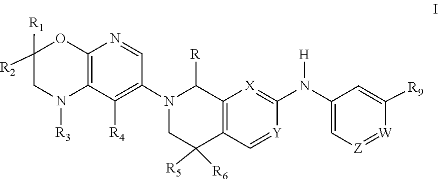
wherein:
R₁ and R₂ are each independently selected from the group consisting of hydrogen and C₁-C₆ alkyl, C₃-C₆ cycloalkyl and C₁-C₆ haloalkyl;
R₁ and R₂ together with the carbon to which they are attached can be taken together to form a C₃-C₆ carbocyclic ring;
R₃ is selected from the group consisting of hydrogen and C₁-C₆ alkyl;
R₄ is selected from the group consisting of hydrogen, halogen, C₁-C₆ alkyl, C₃-C₆ cycloalkyl and C₁-C₆ haloalkyl;
R₅ and R₆ are each independently selected from the group consisting of hydrogen, halogen, C₁-C₆ alkyl, C₃-C₆ cycloalkyl and C₁-C₆ haloalkyl;
R₇ is selected from the group consisting of hydrogen and C₁-C₆ alkyl;
X is selected from the group consisting of N and CH;
Y is selected from the group consisting of N and CH;
W is selected from the group consisting of N and CR₈;
Z is selected from the group consisting of N and CH;
R₈ and R₉ are independently selected from the group consisting of hydrogen, halogen, optionally substituted C₁-C₆ alkyl, optionally substituted C₃-C₆ cycloalkyl, C₁-C₆ haloalkyl, optionally substituted C₂-C₈ heterocyclic, optionally substituted heteroaromatic, optionally substituted C₆-C₁₄ aromatic, —(CH₂)ₘ—NR′R″, —NR′—(CH₂)ₘ—NR′R″, —(CH₂)ₙ—OR₁₁, —O—(CH₂)ₙ—OR₁₁; 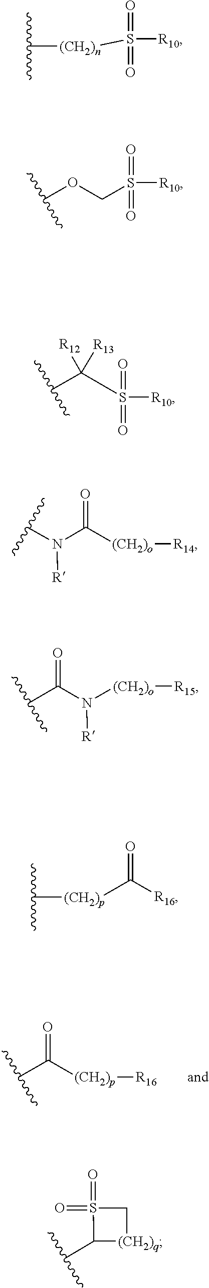
R₁₀ is selected independently for each occurrence from the group consisting C₁-C₆ alkyl, —NR′R″, —(CH₂)ₚ—OR₁₁, —(CH₂)ₙ—O—(CH₂)ₙ—OR₁₁ and —(CH₂)ₘ—NR′R″;
R′ and R″ are selected independently for each occurrence from the group consisting of hydrogen, C₁-C₆ alkyl and C₃-C₆ cycloalkyl;
R₁₂ and R₁₃ are independently selected for each occurrence from the group consisting of hydrogen and C₁-C₆ alkyl; R₁₂ and R₁₃ together with the carbon to which they are attached can be taken together to form a C₃-C₆ carbocyclic ring;
R₁₁, R₁₄, R₁₅ and R₁₆ are independently selected for each occurrence from the group consisting of hydrogen, C₁-C₆ alkyl, optionally substituted C₂-C₈ heterocyclic, optionally substituted heteroaromatic, optionally substituted C₆-C₁₄ aromatic;
m, n, o, p and q are each independently selected from the group consisting of 0, 1, 2, 3, 4, 5 and 6;
or enantiomers or diastereomers or mixtures thereof or pharmaceutically acceptable salts thereof.

2. The compound according to claim 1 wherein R₁ and R₂ are hydrogen.

3. The compound according to claim 1 wherein R₃ is selected from the group consisting of hydrogen and —CH₃.

4. The compound according to claim 1 wherein R₄ is selected from the group consisting of hydrogen, 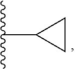 [SMILES: *C1CC1]
Cl, —CH₂CH₃ and —CH₃.

5. The compound according to claim 1 wherein R₅ and R₆ are hydrogen.

6. The compound according to claim 1 wherein R₇ is selected from the group consisting of hydrogen and —CH₃.

7. The compound according to claim 1 wherein X and Y are CH.

8. The compound according to claim 1 wherein X and Y are N.

9. The compound according to claim 1 wherein X is N and Y is CH.

10. The compound according to claim 1 wherein W is CR₈.

11. The compound according to claim 10 wherein R₈ is selected from the group consisting of

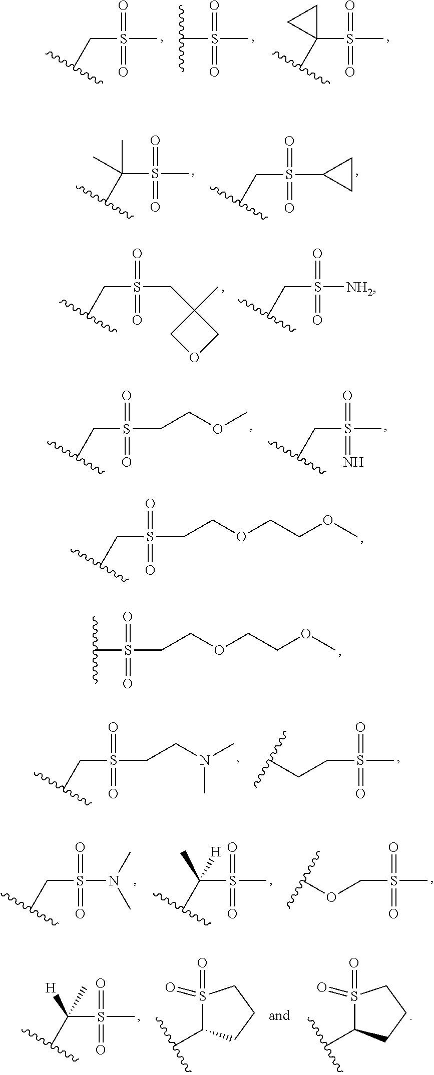

12. The compound according to claim 10 wherein R₈ is selected from the group consisting of

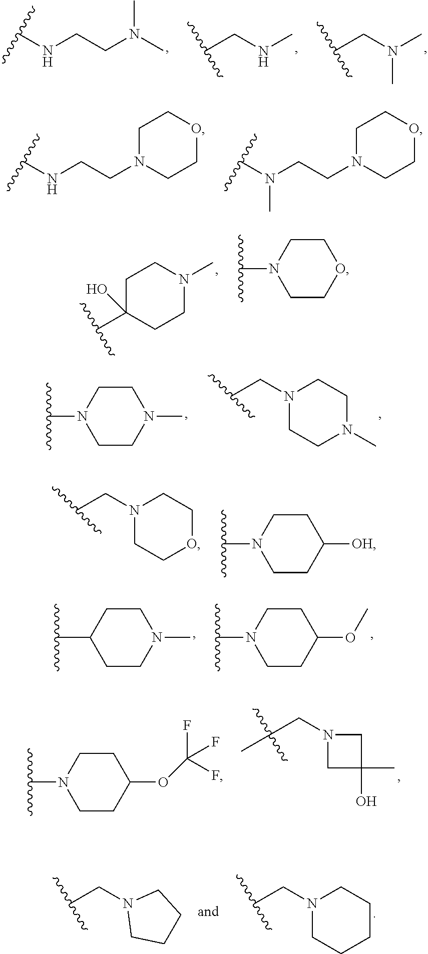

13. The compound according to claim 10 wherein R₈ is selected from the group consisting of

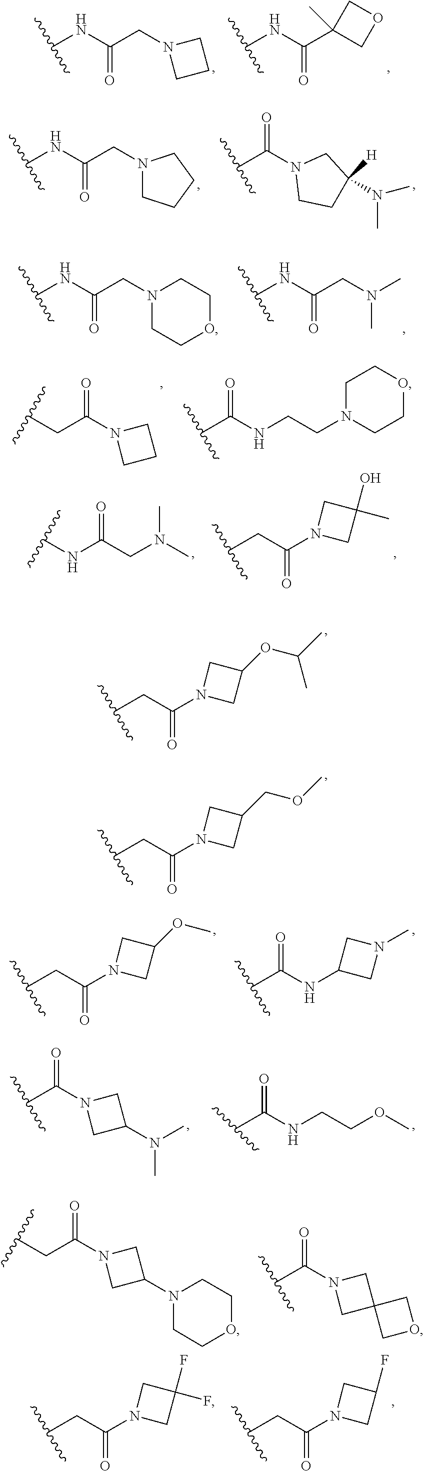

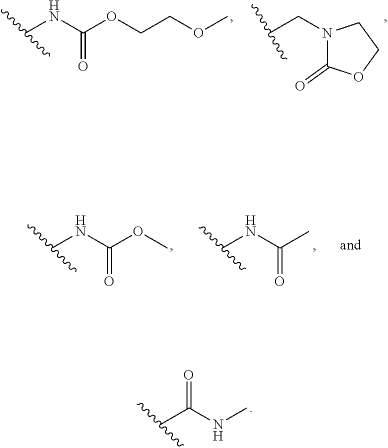

14. The compound according to claim 10 wherein R₈ is selected from the group consisting of

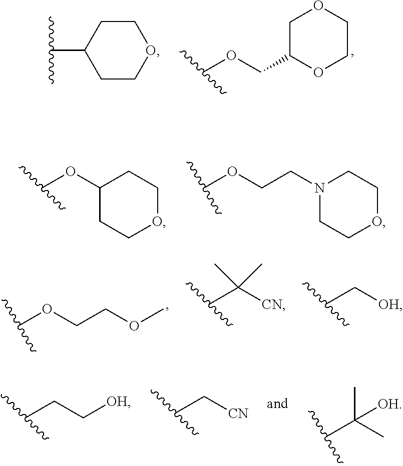

15. The compound according to claim 10 wherein R₈ is selected from the group consisting of

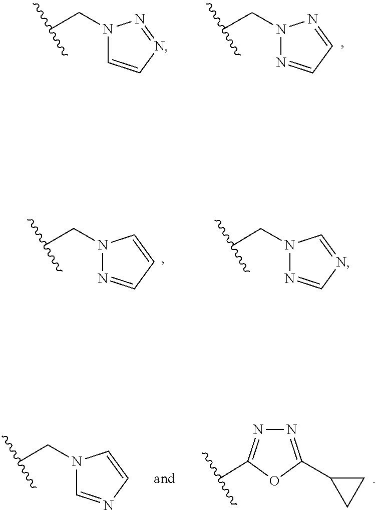

16. The compound according to claim 1 wherein R₉ is selected from the group consisting of
hydrogen, F. Cl, Br, I, —CF₃, —CH₃, —CH₂CH₃,

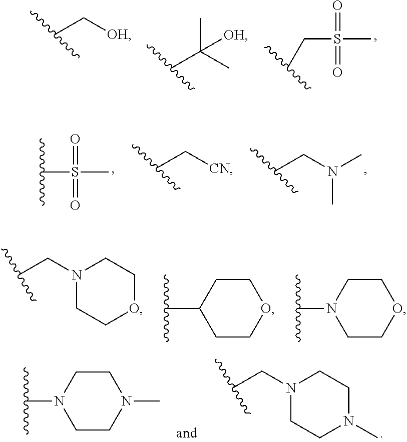

17-47. (canceled)

48. A compound according to claim 1 selected from the group consisting of 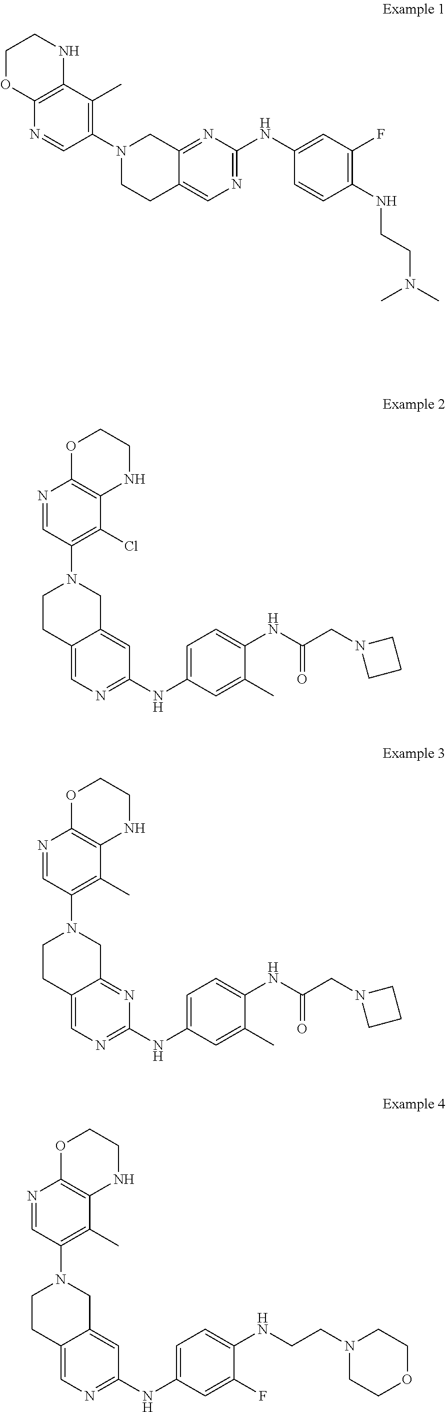 [SMILES: Cc1c(N2CCc3cnc(Nc4ccc(NCCN(C)C)c(F)c4)nc3C2)cnc2c1NCCO2 ; Cc1cc(Nc2cc3c(cn2)CCN(c2cnc4c(c2Cl)NCCO4)C3)ccc1NC(=O)CN1CCC1 ; Cc1cc(Nc2ncc3c(n2)CN(c2cnc4c(c2C)NCCO4)CC3)ccc1NC(=O)CN1CCC1 ; Cc1c(N2CCc3cnc(Nc4ccc(NCCN5CCOCC5)c(F)c4)cc3C2)cnc2c1NCCO2] 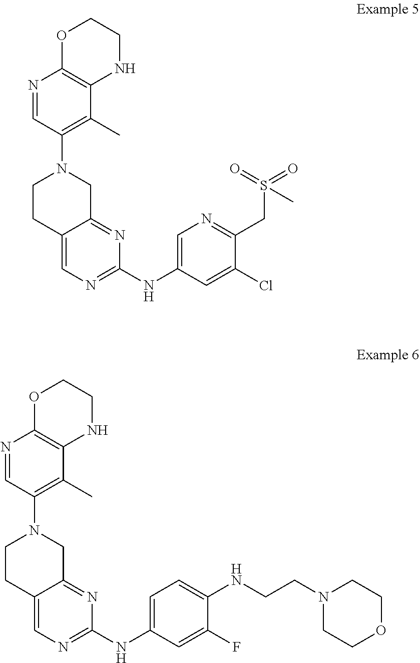 [SMILES: Cc1c(N2CCc3cnc(Nc4cnc(CS(C)(=O)=O)c(Cl)c4)nc3C2)cnc2c1NCCO2 ; Cc1c(N2CCc3cnc(Nc4ccc(NCCN5CCOCC5)c(F)c4)nc3C2)cnc2c1NCCO2] 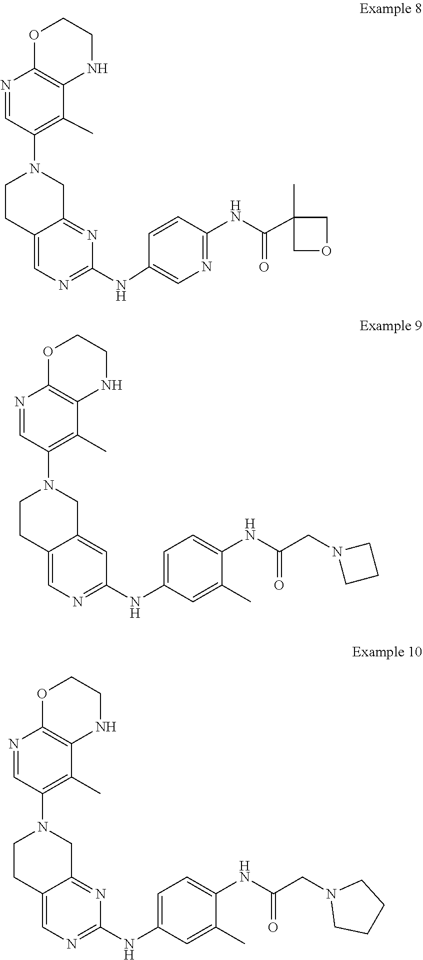 [SMILES: Cc1c(N2CCc3cnc(Nc4ccc(NC(=O)C5(C)COC5)nc4)nc3C2)cnc2c1NCCO2 ; Cc1cc(Nc2cc3c(cn2)CCN(c2cnc4c(c2C)NCCO4)C3)ccc1NC(=O)CN1CCC1 ; Cc1cc(Nc2ncc3c(n2)CN(c2cnc4c(c2C)NCCO4)CC3)ccc1NC(=O)CN1CCCC1] 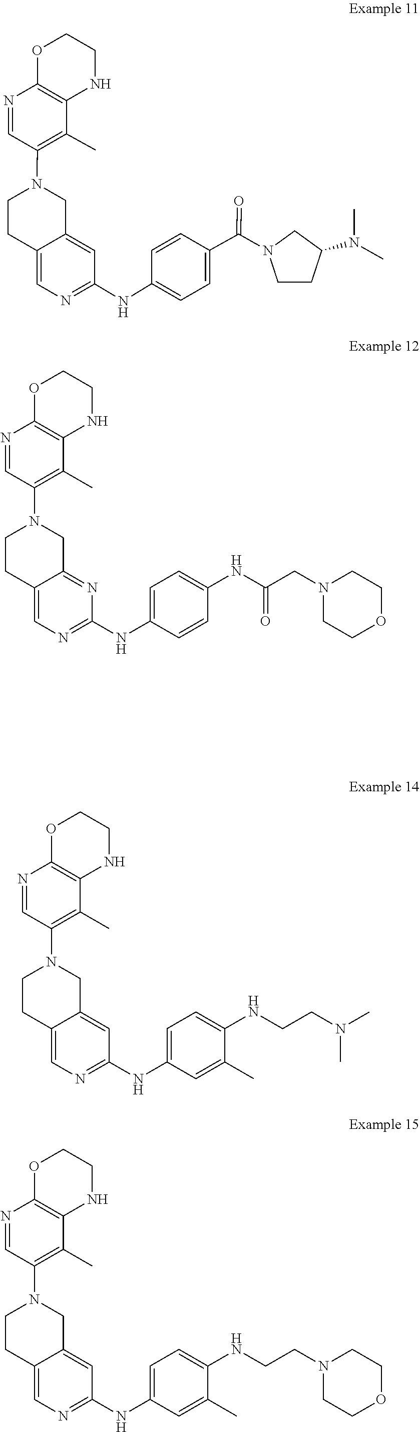 [SMILES: Cc1c(N2CCc3cnc(Nc4ccc(C(=O)N5CC[C@@H](N(C)C)C5)cc4)cc3C2)cnc2c1NCCO2 ; Cc1c(N2CCc3cnc(Nc4ccc(NC(=O)CN5CCOCC5)cc4)nc3C2)cnc2c1NCCO2 ; Cc1cc(Nc2cc3c(cn2)CCN(c2cnc4c(c2C)NCCO4)C3)ccc1NCCN(C)C ; Cc1cc(Nc2cc3c(cn2)CCN(c2cnc4c(c2C)NCCO4)C3)ccc1NCCN1CCOCC1] 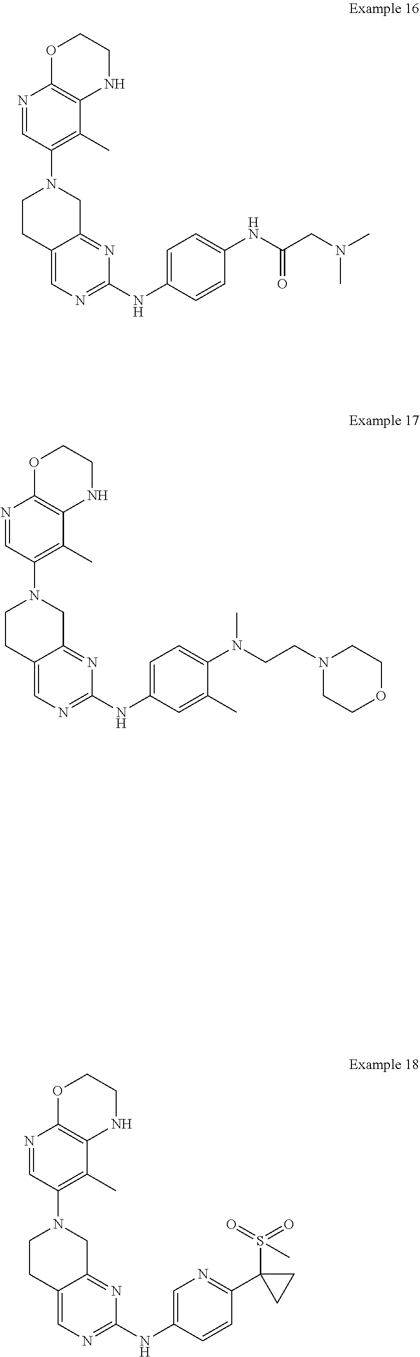 [SMILES: Cc1c(N2CCc3cnc(Nc4ccc(NC(=O)CN(C)C)cc4)nc3C2)cnc2c1NCCO2 ; Cc1cc(Nc2ncc3c(n2)CN(c2cnc4c(c2C)NCCO4)CC3)ccc1N(C)CCN1CCOCC1 ; Cc1c(N2CCc3cnc(Nc4ccc(C5(S(C)(=O)=O)CC5)nc4)nc3C2)cnc2c1NCCO2] 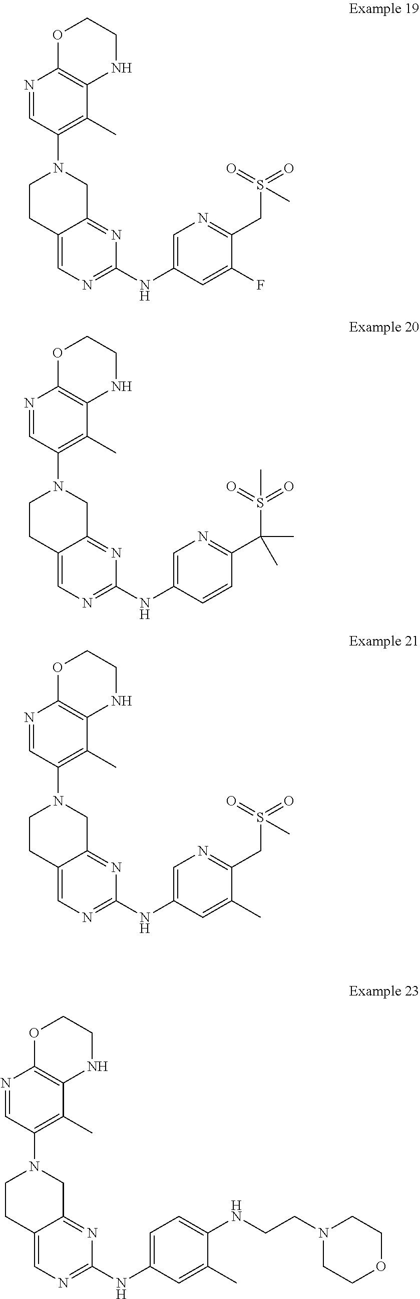 [SMILES: Cc1c(N2CCc3cnc(Nc4cnc(CS(C)(=O)=O)c(F)c4)nc3C2)cnc2c1NCCO2 ; Cc1c(N2CCc3cnc(Nc4ccc(C(C)(C)S(C)(=O)=O)nc4)nc3C2)cnc2c1NCCO2 ; Cc1cc(Nc2ncc3c(n2)CN(c2cnc4c(c2C)NCCO4)CC3)cnc1CS(C)(=O)=O ; Cc1cc(Nc2ncc3c(n2)CN(c2cnc4c(c2C)NCCO4)CC3)ccc1NCCN1CCOCC1] 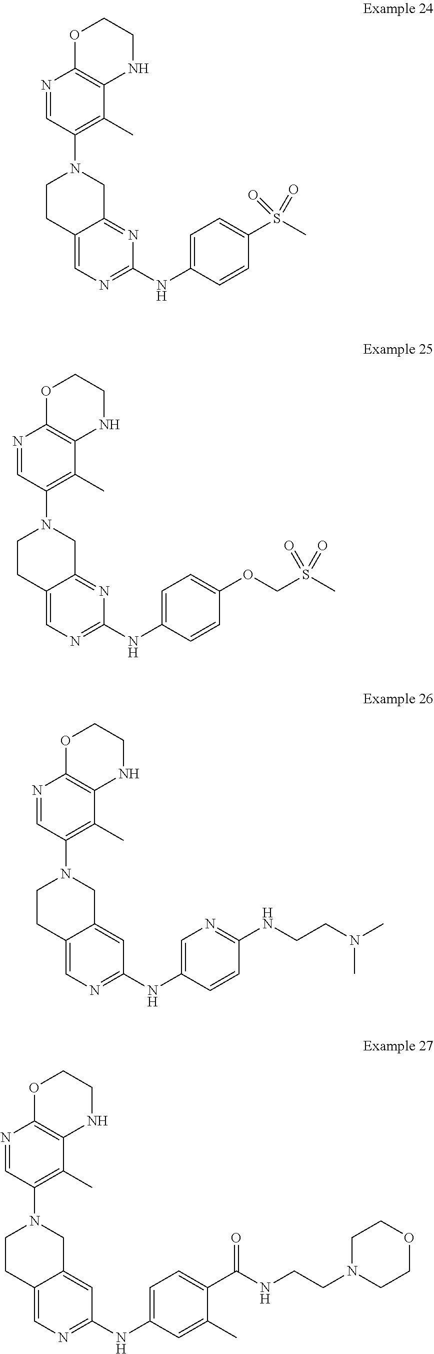 [SMILES: Cc1c(N2CCc3cnc(Nc4ccc(S(C)(=O)=O)cc4)nc3C2)cnc2c1NCCO2 ; Cc1c(N2CCc3cnc(Nc4ccc(OCS(C)(=O)=O)cc4)nc3C2)cnc2c1NCCO2 ; Cc1c(N2CCc3cnc(Nc4ccc(NCCN(C)C)nc4)cc3C2)cnc2c1NCCO2 ; Cc1cc(Nc2cc3c(cn2)CCN(c2cnc4c(c2C)NCCO4)C3)ccc1C(=O)NCCN1CCOCC1] 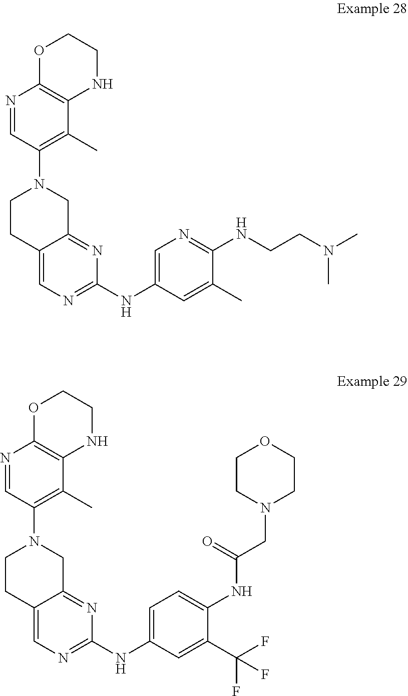 [SMILES: Cc1cc(Nc2ncc3c(n2)CN(c2cnc4c(c2C)NCCO4)CC3)cnc1NCCN(C)C ; Cc1c(N2CCc3cnc(Nc4ccc(NC(=O)CN5CCOCC5)c(C(F)(F)F)c4)nc3C2)cnc2c1NCCO2] 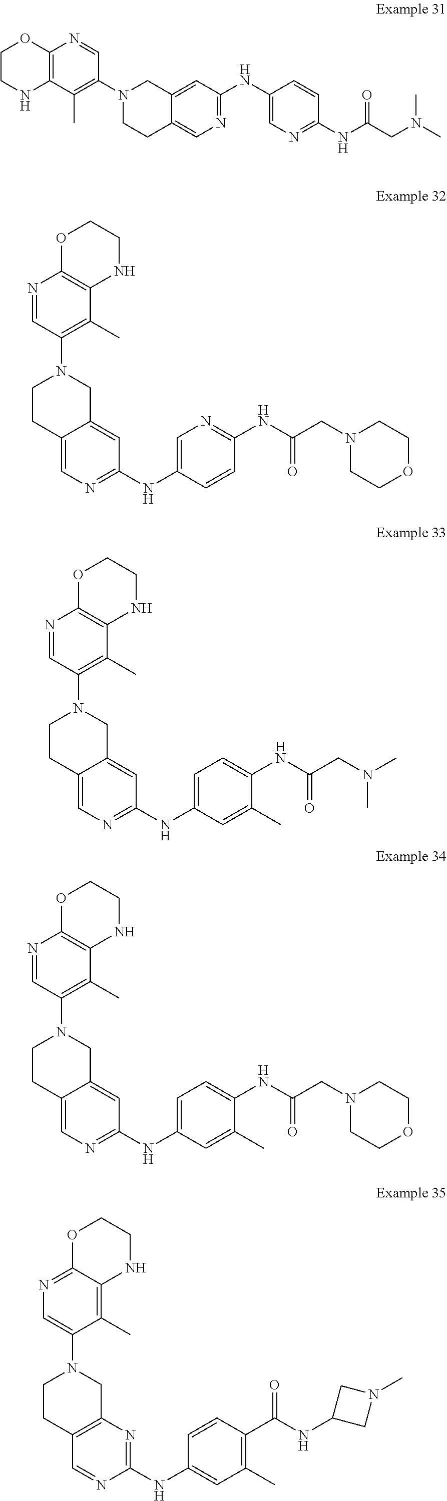 [SMILES: Cc1c(N2CCc3cnc(Nc4ccc(NC(=O)CN(C)C)nc4)cc3C2)cnc2c1NCCO2 ; Cc1c(N2CCc3cnc(Nc4ccc(NC(=O)CN5CCOCC5)nc4)cc3C2)cnc2c1NCCO2 ; Cc1cc(Nc2cc3c(cn2)CCN(c2cnc4c(c2C)NCCO4)C3)ccc1NC(=O)CN(C)C ; Cc1cc(Nc2cc3c(cn2)CCN(c2cnc4c(c2C)NCCO4)C3)ccc1NC(=O)CN1CCOCC1 ; Cc1cc(Nc2ncc3c(n2)CN(c2cnc4c(c2C)NCCO4)CC3)ccc1C(=O)NC1CN(C)C1] 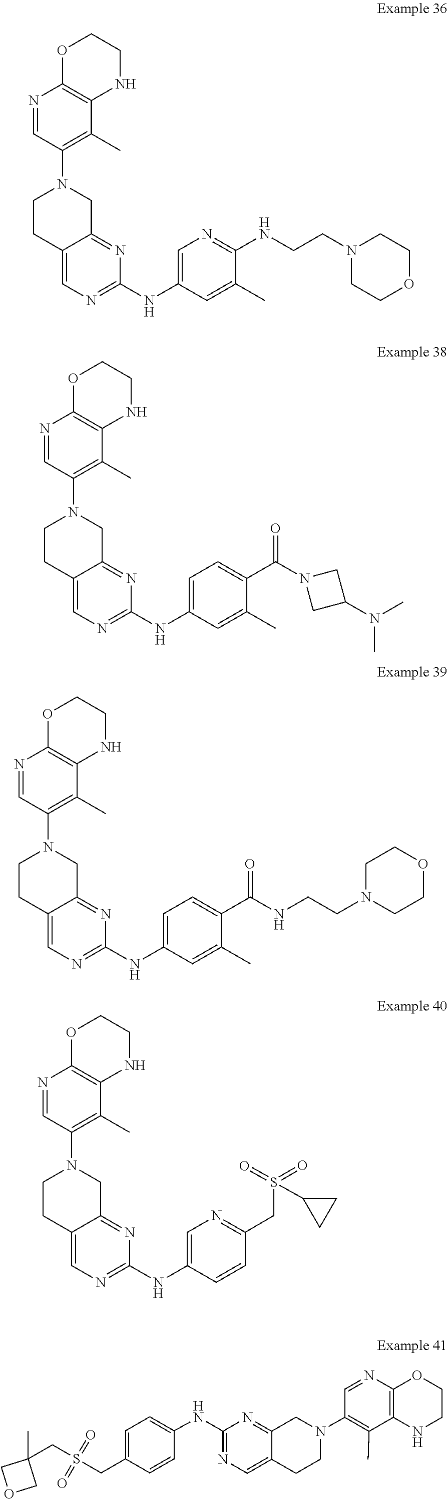 [SMILES: Cc1cc(Nc2ncc3c(n2)CN(c2cnc4c(c2C)NCCO4)CC3)cnc1NCCN1CCOCC1 ; Cc1cc(Nc2ncc3c(n2)CN(c2cnc4c(c2C)NCCO4)CC3)ccc1C(=O)N1CC(N(C)C)C1 ; Cc1cc(Nc2ncc3c(n2)CN(c2cnc4c(c2C)NCCO4)CC3)ccc1C(=O)NCCN1CCOCC1 ; Cc1c(N2CCc3cnc(Nc4ccc(CS(=O)(=O)C5CC5)nc4)nc3C2)cnc2c1NCCO2 ; Cc1c(N2CCc3cnc(Nc4ccc(CS(=O)(=O)CC5(C)COC5)cc4)nc3C2)cnc2c1NCCO2] 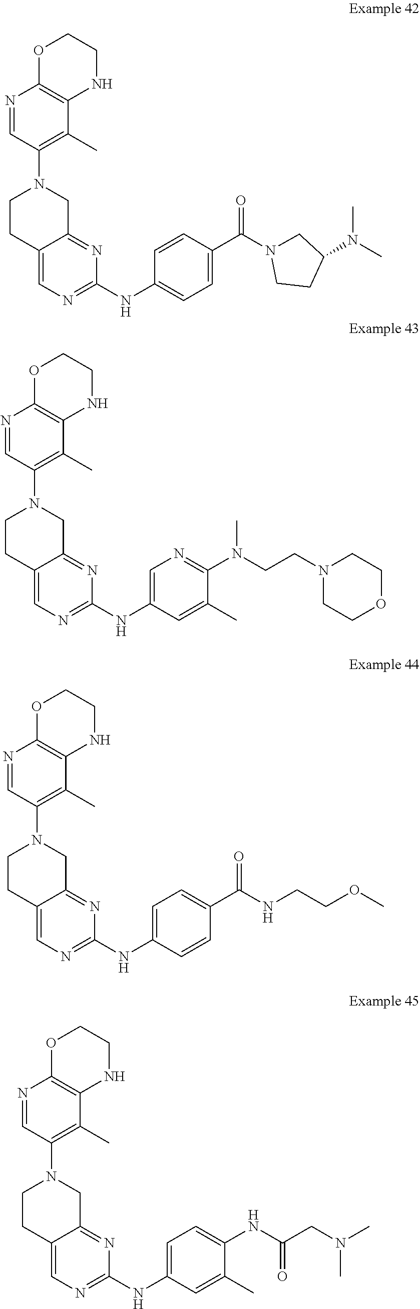 [SMILES: Cc1c(N2CCc3cnc(Nc4ccc(C(=O)N5CC[C@@H](N(C)C)C5)cc4)nc3C2)cnc2c1NCCO2 ; Cc1cc(Nc2ncc3c(n2)CN(c2cnc4c(c2C)NCCO4)CC3)cnc1N(C)CCN1CCOCC1 ; COCCNC(=O)c1ccc(Nc2ncc3c(n2)CN(c2cnc4c(c2C)NCCO4)CC3)cc1 ; Cc1cc(Nc2ncc3c(n2)CN(c2cnc4c(c2C)NCCO4)CC3)ccc1NC(=O)CN(C)C] 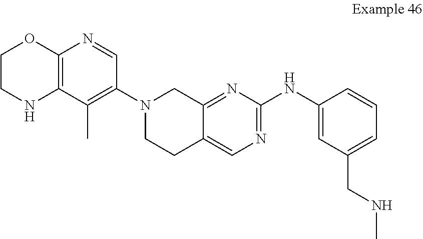 [SMILES: CNCc1cccc(Nc2ncc3c(n2)CN(c2cnc4c(c2C)NCCO4)CC3)c1] 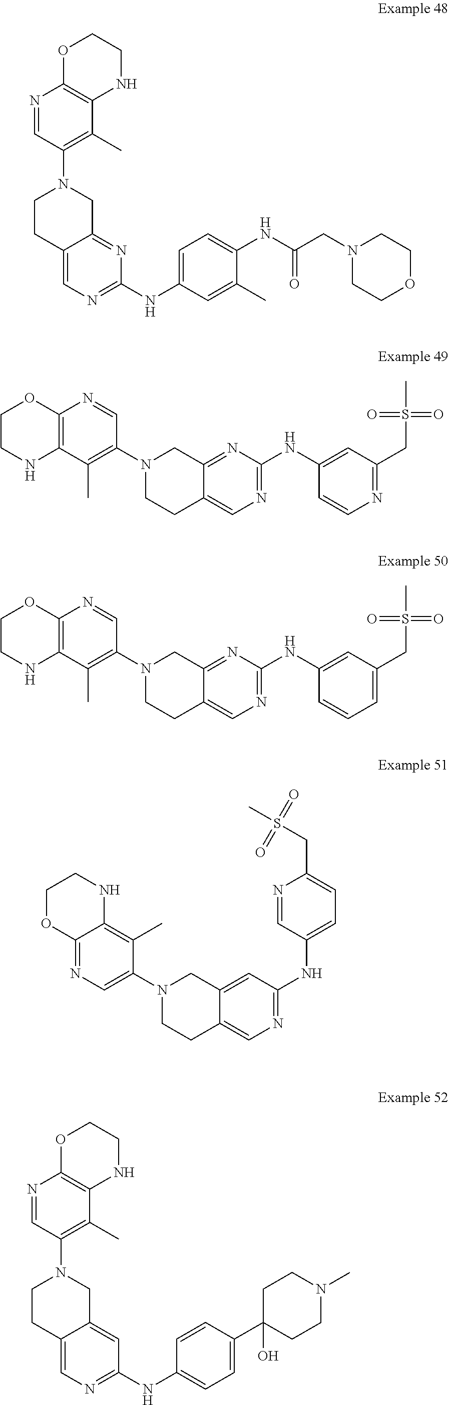 [SMILES: Cc1cc(Nc2ncc3c(n2)CN(c2cnc4c(c2C)NCCO4)CC3)ccc1NC(=O)CN1CCOCC1 ; Cc1c(N2CCc3cnc(Nc4ccnc(CS(C)(=O)=O)c4)nc3C2)cnc2c1NCCO2 ; Cc1c(N2CCc3cnc(Nc4cccc(CS(C)(=O)=O)c4)nc3C2)cnc2c1NCCO2 ; Cc1c(N2CCc3cnc(Nc4ccc(CS(C)(=O)=O)nc4)cc3C2)cnc2c1NCCO2 ; Cc1c(N2CCc3cnc(Nc4ccc(C5(O)CCN(C)CC5)cc4)cc3C2)cnc2c1NCCO2] 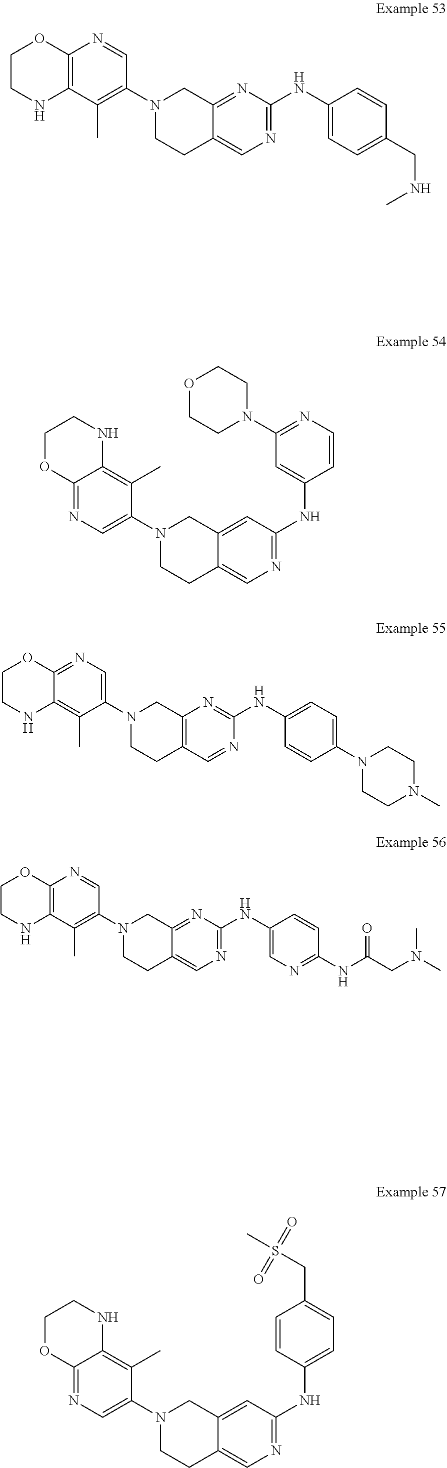 [SMILES: CNCc1ccc(Nc2ncc3c(n2)CN(c2cnc4c(c2C)NCCO4)CC3)cc1 ; Cc1c(N2CCc3cnc(Nc4ccnc(N5CCOCC5)c4)cc3C2)cnc2c1NCCO2 ; Cc1c(N2CCc3cnc(Nc4ccc(N5CCN(C)CC5)cc4)nc3C2)cnc2c1NCCO2 ; Cc1c(N2CCc3cnc(Nc4ccc(NC(=O)CN(C)C)nc4)nc3C2)cnc2c1NCCO2 ; Cc1c(N2CCc3cnc(Nc4ccc(CS(C)(=O)=O)cc4)cc3C2)cnc2c1NCCO2] 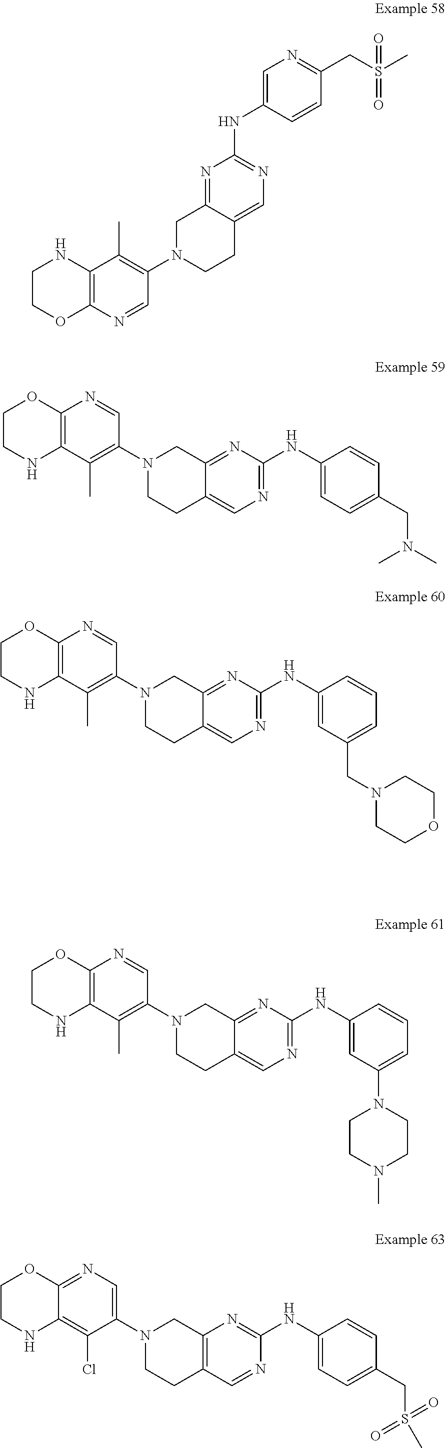 [SMILES: Cc1c(N2CCc3cnc(Nc4ccc(CS(C)(=O)=O)nc4)nc3C2)cnc2c1NCCO2 ; Cc1c(N2CCc3cnc(Nc4ccc(CN(C)C)cc4)nc3C2)cnc2c1NCCO2 ; Cc1c(N2CCc3cnc(Nc4cccc(CN5CCOCC5)c4)nc3C2)cnc2c1NCCO2 ; Cc1c(N2CCc3cnc(Nc4cccc(N5CCN(C)CC5)c4)nc3C2)cnc2c1NCCO2 ; CS(=O)(=O)Cc1ccc(Nc2ncc3c(n2)CN(c2cnc4c(c2Cl)NCCO4)CC3)cc1] 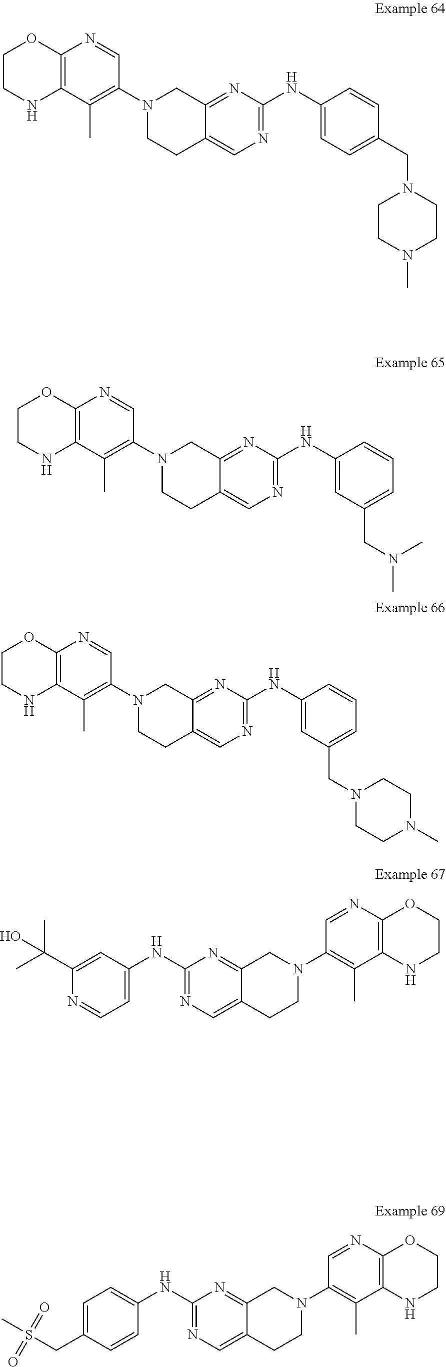 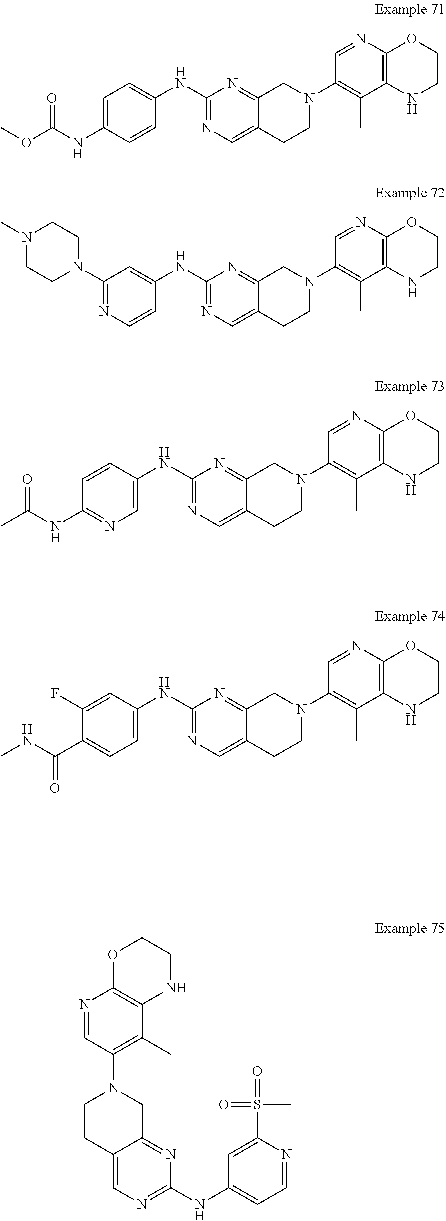 [SMILES: COC(=O)Nc1ccc(Nc2ncc3c(n2)CN(c2cnc4c(c2C)NCCO4)CC3)cc1 ; Cc1c(N2CCc3cnc(Nc4ccnc(N5CCN(C)CC5)c4)nc3C2)cnc2c1NCCO2 ; CC(=O)Nc1ccc(Nc2ncc3c(n2)CN(c2cnc4c(c2C)NCCO4)CC3)cn1 ; CNC(=O)c1ccc(Nc2ncc3c(n2)CN(c2cnc4c(c2C)NCCO4)CC3)cc1F ; Cc1c(N2CCc3cnc(Nc4ccnc(S(C)(=O)=O)c4)nc3C2)cnc2c1NCCO2] 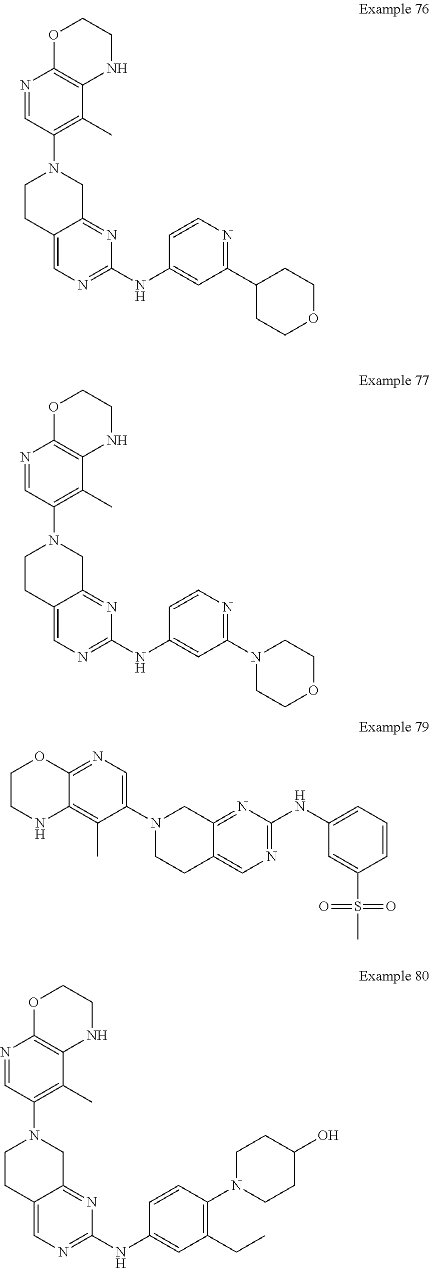 [SMILES: Cc1c(N2CCc3cnc(Nc4ccnc(C5CCOCC5)c4)nc3C2)cnc2c1NCCO2 ; Cc1c(N2CCc3cnc(Nc4ccnc(N5CCOCC5)c4)nc3C2)cnc2c1NCCO2 ; Cc1c(N2CCc3cnc(Nc4cccc(S(C)(=O)=O)c4)nc3C2)cnc2c1NCCO2 ; CCc1cc(Nc2ncc3c(n2)CN(c2cnc4c(c2C)NCCO4)CC3)ccc1N1CCC(O)CC1] 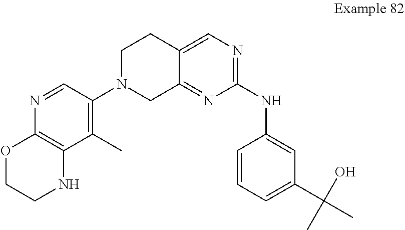 [SMILES: Cc1c(N2CCc3cnc(Nc4cccc(C(C)(C)O)c4)nc3C2)cnc2c1NCCO2] 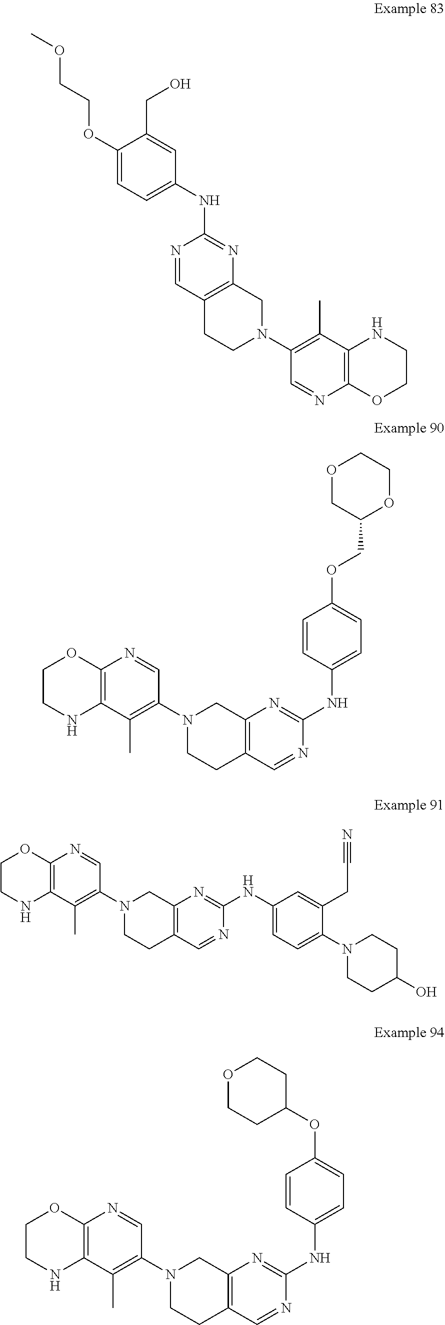 [SMILES: COCCOc1ccc(Nc2ncc3c(n2)CN(c2cnc4c(c2C)NCCO4)CC3)cc1CO ; Cc1c(N2CCc3cnc(Nc4ccc(OC[C@@H]5COCCO5)cc4)nc3C2)cnc2c1NCCO2 ; Cc1c(N2CCc3cnc(Nc4ccc(N5CCC(O)CC5)c(CC#N)c4)nc3C2)cnc2c1NCCO2 ; Cc1c(N2CCc3cnc(Nc4ccc(OC5CCOCC5)cc4)nc3C2)cnc2c1NCCO2] 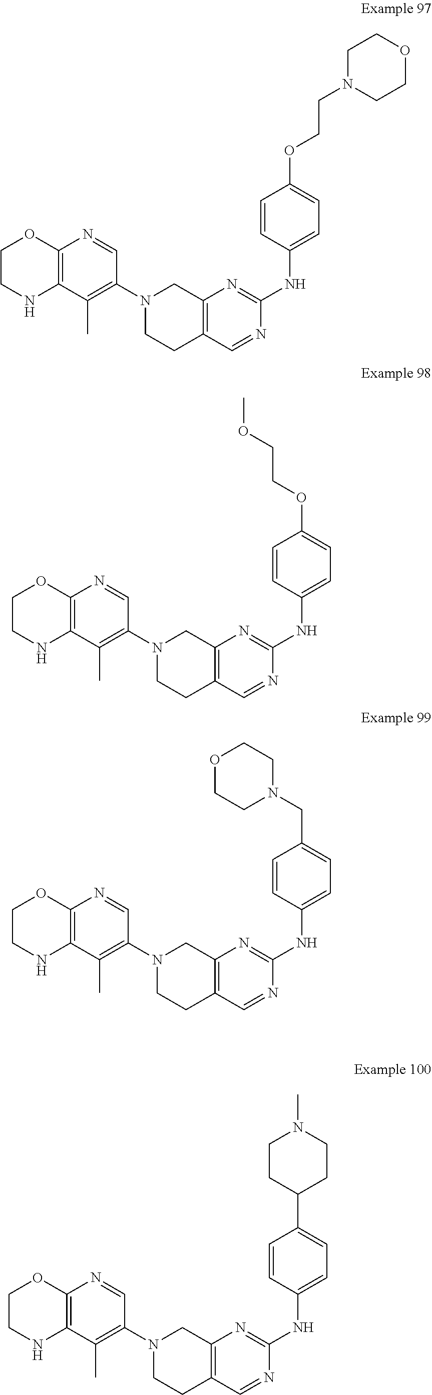 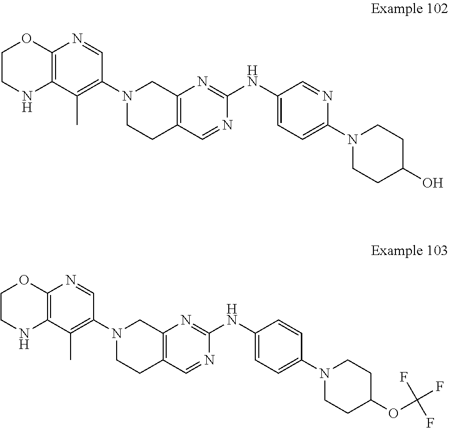 [SMILES: Cc1c(N2CCc3cnc(Nc4ccc(N5CCC(O)CC5)nc4)nc3C2)cnc2c1NCCO2 ; Cc1c(N2CCc3cnc(Nc4ccc(N5CCC(OC(F)(F)F)CC5)cc4)nc3C2)cnc2c1NCCO2] 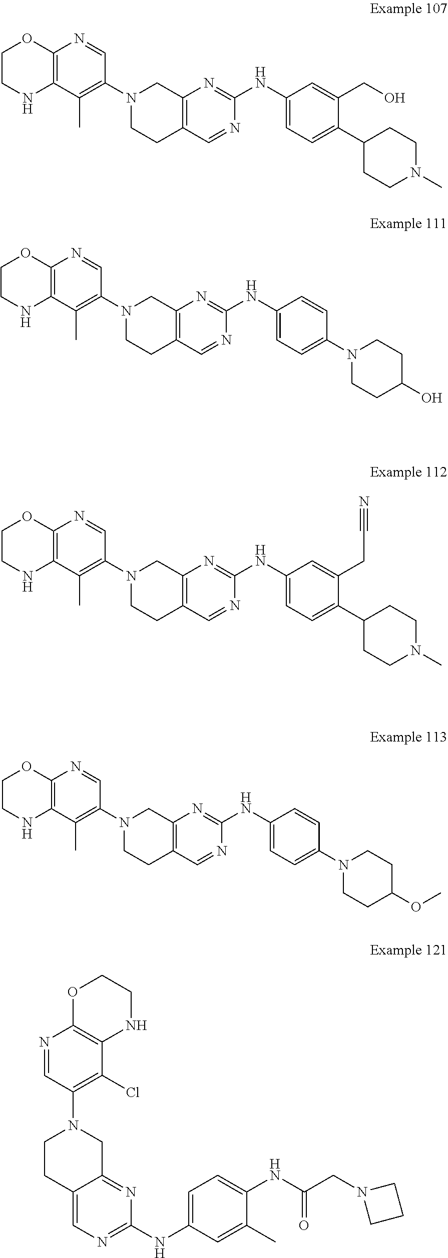 [SMILES: Cc1c(N2CCc3cnc(Nc4ccc(C5CCN(C)CC5)c(CO)c4)nc3C2)cnc2c1NCCO2 ; Cc1c(N2CCc3cnc(Nc4ccc(N5CCC(O)CC5)cc4)nc3C2)cnc2c1NCCO2 ; Cc1c(N2CCc3cnc(Nc4ccc(C5CCN(C)CC5)c(CC#N)c4)nc3C2)cnc2c1NCCO2 ; COC1CCN(c2ccc(Nc3ncc4c(n3)CN(c3cnc5c(c3C)NCCO5)CC4)cc2)CC1 ; Cc1cc(Nc2ncc3c(n2)CN(c2cnc4c(c2Cl)NCCO4)CC3)ccc1NC(=O)CN1CCC1] 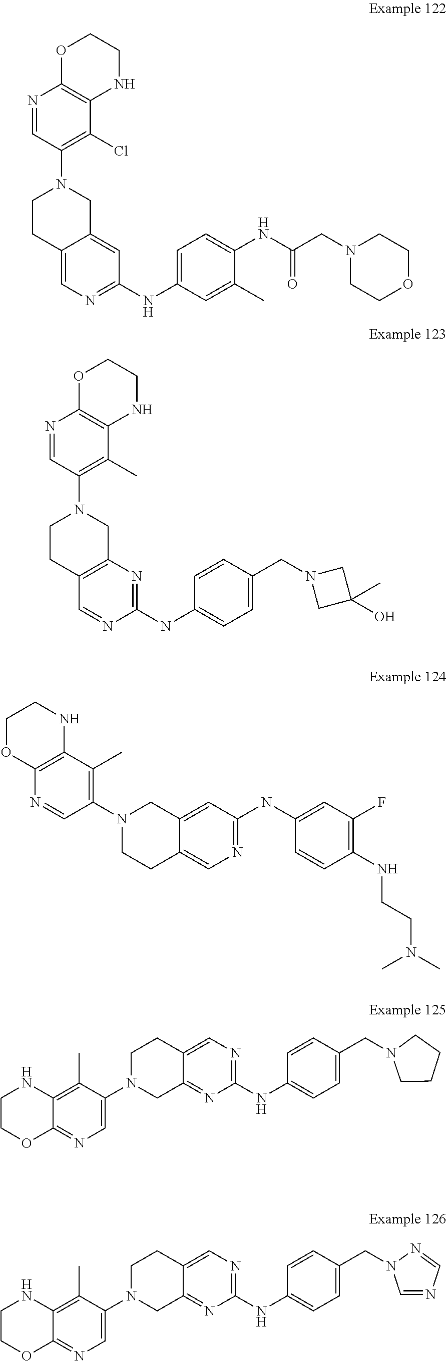 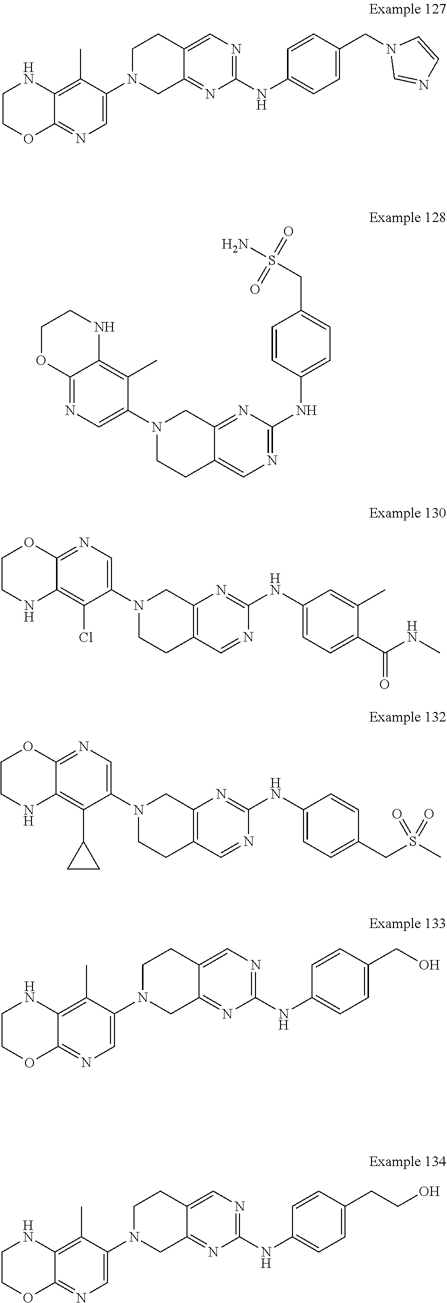 [SMILES: Cc1c(N2CCc3cnc(Nc4ccc(Cn5ccnc5)cc4)nc3C2)cnc2c1NCCO2 ; Cc1c(N2CCc3cnc(Nc4ccc(CS(N)(=O)=O)cc4)nc3C2)cnc2c1NCCO2 ; CNC(=O)c1ccc(Nc2ncc3c(n2)CN(c2cnc4c(c2Cl)NCCO4)CC3)cc1C ; CS(=O)(=O)Cc1ccc(Nc2ncc3c(n2)CN(c2cnc4c(c2C2CC2)NCCO4)CC3)cc1 ; Cc1c(N2CCc3cnc(Nc4ccc(CO)cc4)nc3C2)cnc2c1NCCO2 ; Cc1c(N2CCc3cnc(Nc4ccc(CCO)cc4)nc3C2)cnc2c1NCCO2] 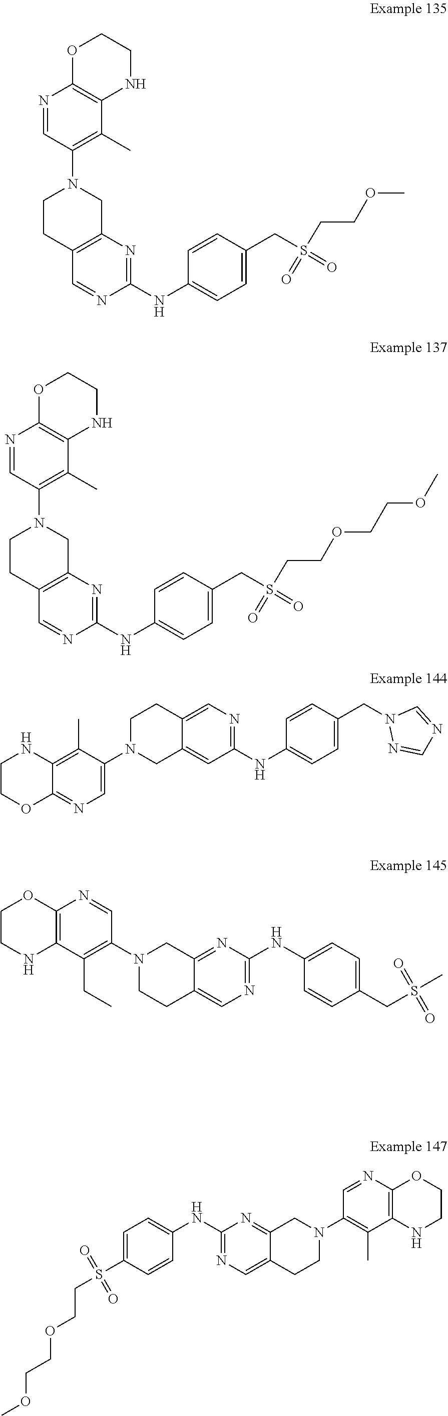 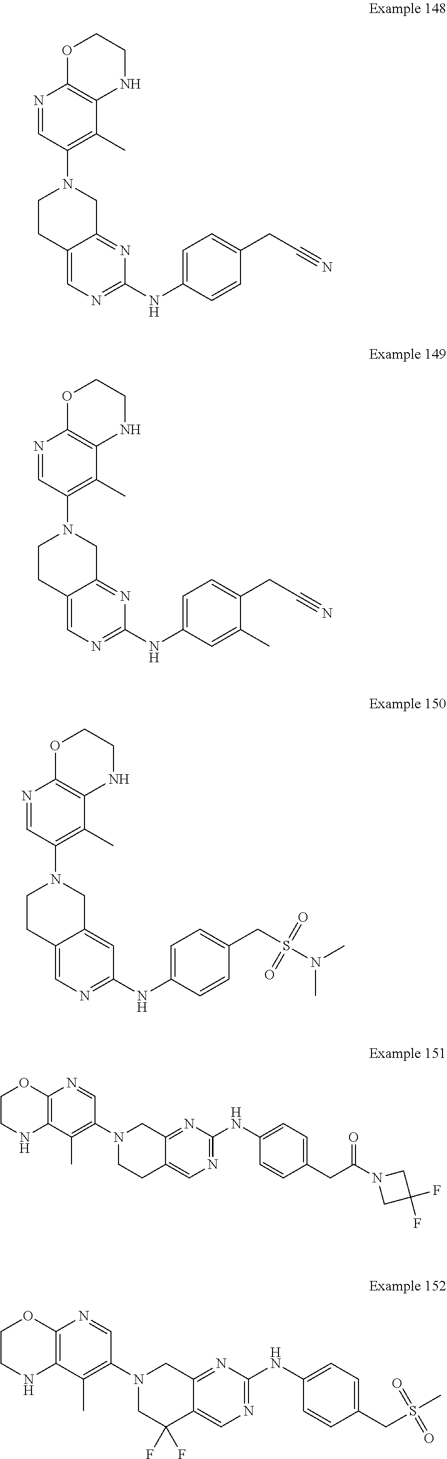 [SMILES: Cc1c(N2CCc3cnc(Nc4ccc(CC#N)cc4)nc3C2)cnc2c1NCCO2 ; Cc1cc(Nc2ncc3c(n2)CN(c2cnc4c(c2C)NCCO4)CC3)ccc1CC#N ; Cc1c(N2CCc3cnc(Nc4ccc(CS(=O)(=O)N(C)C)cc4)cc3C2)cnc2c1NCCO2 ; Cc1c(N2CCc3cnc(Nc4ccc(CC(=O)N5CC(F)(F)C5)cc4)nc3C2)cnc2c1NCCO2 ; Cc1c(N2Cc3nc(Nc4ccc(CS(C)(=O)=O)cc4)ncc3C(F)(F)C2)cnc2c1NCCO2] 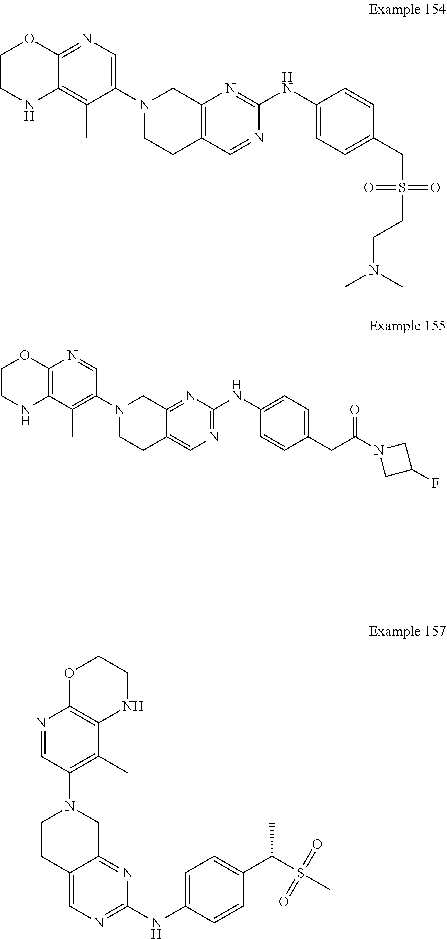 [SMILES: Cc1c(N2CCc3cnc(Nc4ccc(CS(=O)(=O)CCN(C)C)cc4)nc3C2)cnc2c1NCCO2 ; Cc1c(N2CCc3cnc(Nc4ccc(CC(=O)N5CC(F)C5)cc4)nc3C2)cnc2c1NCCO2 ; Cc1c(N2CCc3cnc(Nc4ccc([C@H](C)S(C)(=O)=O)cc4)nc3C2)cnc2c1NCCO2] 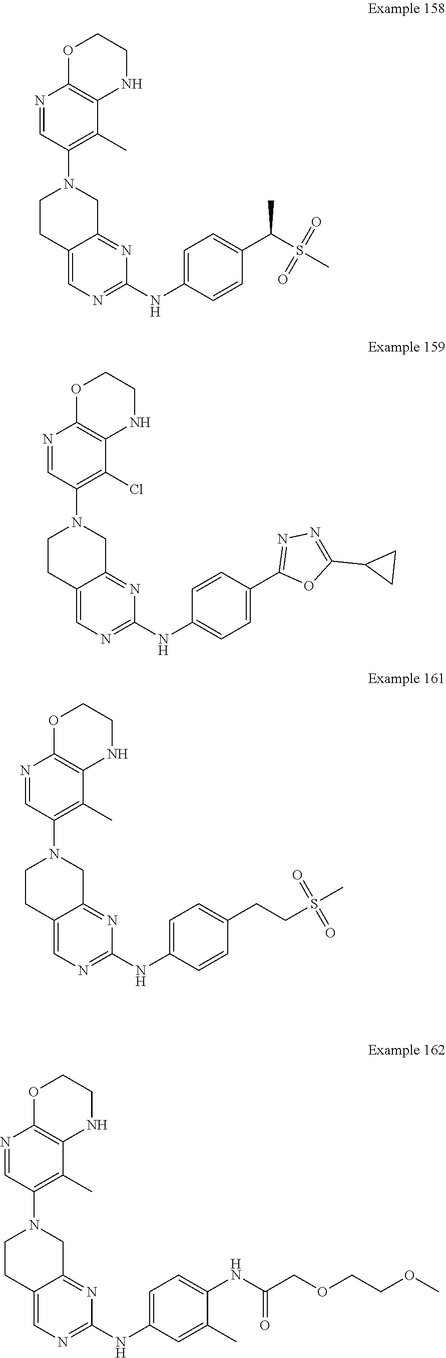 [SMILES: Cc1c(N2CCc3cnc(Nc4ccc([C@@H](C)S(C)(=O)=O)cc4)nc3C2)cnc2c1NCCO2 ; Clc1c(N2CCc3cnc(Nc4ccc(-c5nnc(C6CC6)o5)cc4)nc3C2)cnc2c1NCCO2 ; Cc1c(N2CCc3cnc(Nc4ccc(CCS(C)(=O)=O)cc4)nc3C2)cnc2c1NCCO2 ; COCCOCC(=O)Nc1ccc(Nc2ncc3c(n2)CN(c2cnc4c(c2C)NCCO4)CC3)cc1C] 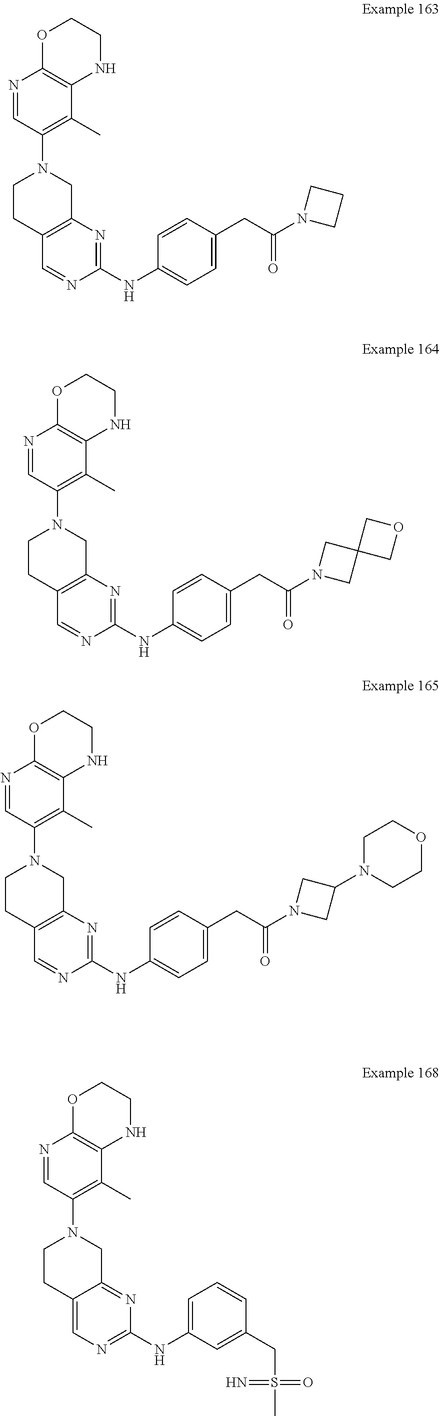 [SMILES: Cc1c(N2CCc3cnc(Nc4ccc(CC(=O)N5CCC5)cc4)nc3C2)cnc2c1NCCO2 ; Cc1c(N2CCc3cnc(Nc4ccc(CC(=O)N5CC6(COC6)C5)cc4)nc3C2)cnc2c1NCCO2 ; Cc1c(N2CCc3cnc(Nc4ccc(CC(=O)N5CC(N6CCOCC6)C5)cc4)nc3C2)cnc2c1NCCO2 ; Cc1c(N2CCc3cnc(Nc4cccc(CS(C)(=N)=O)c4)nc3C2)cnc2c1NCCO2] 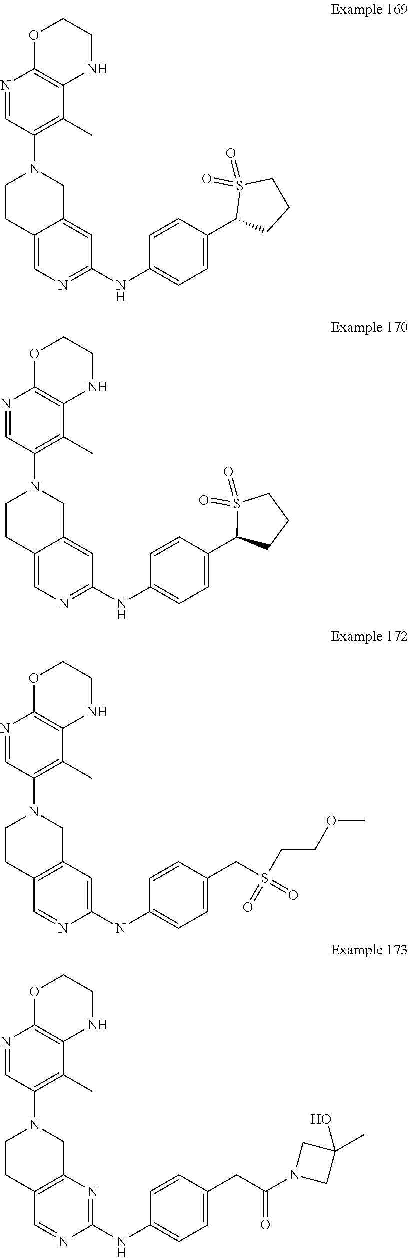 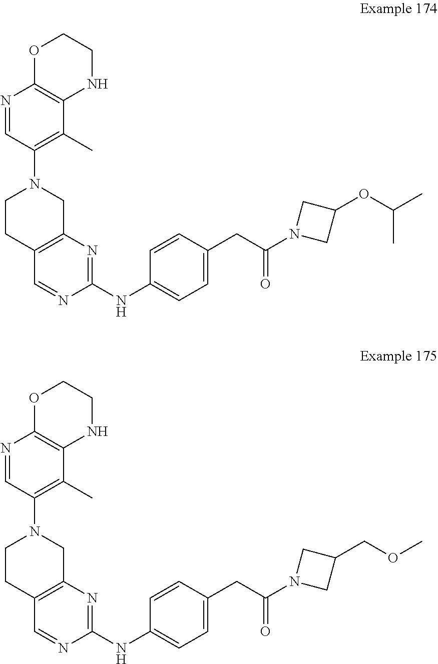 [SMILES: Cc1c(N2CCc3cnc(Nc4ccc(CC(=O)N5CC(OC(C)C)C5)cc4)nc3C2)cnc2c1NCCO2 ; COCC1CN(C(=O)Cc2ccc(Nc3ncc4c(n3)CN(c3cnc5c(c3C)NCCO5)CC4)cc2)C1] 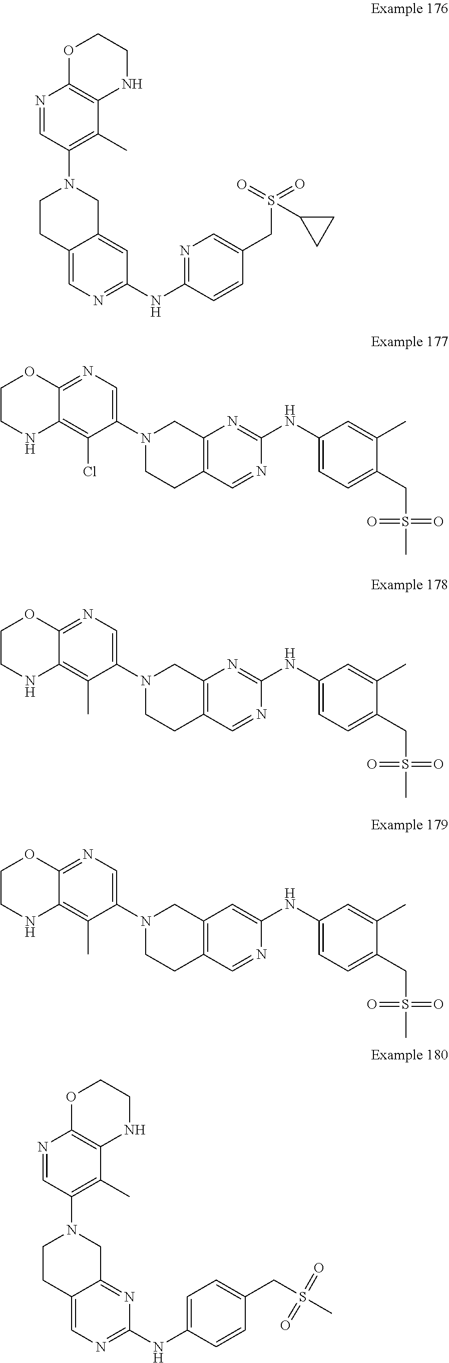 [SMILES: Cc1c(N2CCc3cnc(Nc4ccc(CS(=O)(=O)C5CC5)cn4)cc3C2)cnc2c1NCCO2 ; Cc1cc(Nc2ncc3c(n2)CN(c2cnc4c(c2Cl)NCCO4)CC3)ccc1CS(C)(=O)=O ; Cc1cc(Nc2ncc3c(n2)CN(c2cnc4c(c2C)NCCO4)CC3)ccc1CS(C)(=O)=O ; Cc1cc(Nc2cc3c(cn2)CCN(c2cnc4c(c2C)NCCO4)C3)ccc1CS(C)(=O)=O ; Cc1c(N2CCc3cnc(Nc4ccc(CS(C)(=O)=O)cc4)nc3C2)cnc2c1NCCO2] 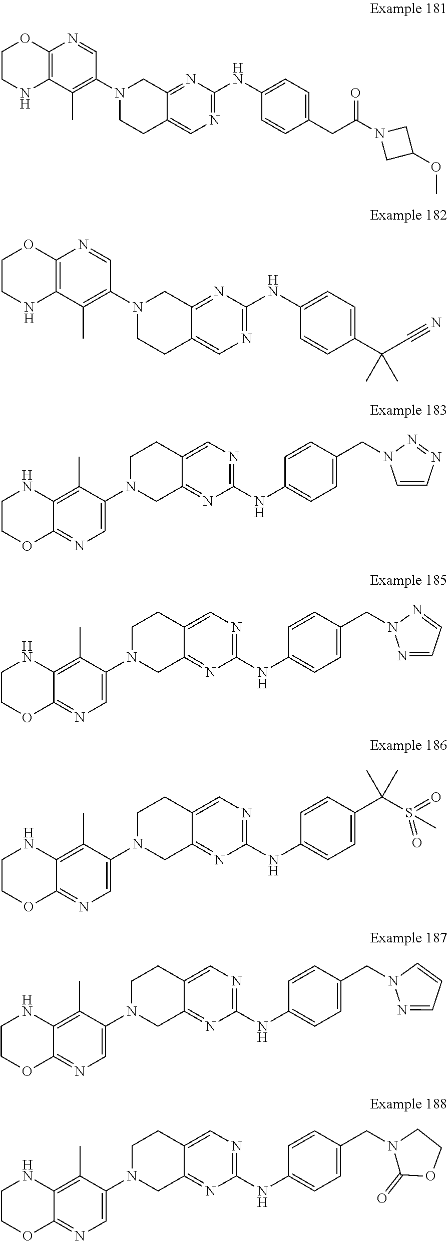 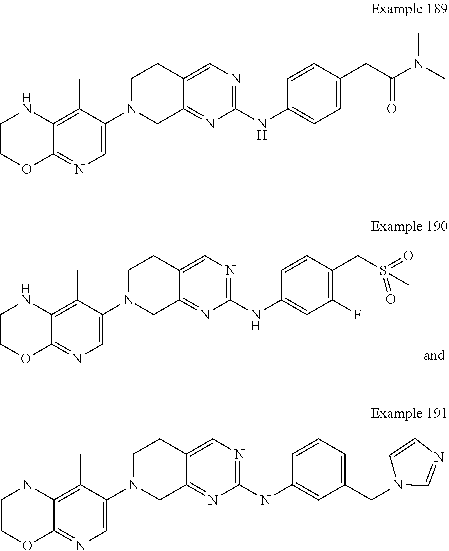
and pharmaceutically acceptable salts thereof.

49-52. (canceled)

53. A pharmaceutical composition comprising a compound according to claim 1 and pharmaceutically acceptable adjuvant, carrier, or vehicle.

54. A method, comprising:
administering to a patient having an HPK1-mediated disorder a therapeutically effective amount of the compound according to claim 1 or a pharmaceutically acceptable salt thereof.

55. The method according to claim 54, wherein the HPK1-mediated disorder is a cancer, autoimmune and/or inflammatory disease.

56. The method according to claim 55, wherein the cancer is selected from the group consisting of cancer of the breast, bladder, bone, brain, central and peripheral nervous system, colon, endocrine glands, esophagus, endometrium, germ cells, head and neck, kidney, liver, lung, larynx and hypopharynx, ovary, pancreas, prostate, rectum, renal, small intestine, soft tissue, testis, stomach, skin, ureter, vagina, and vulva.

57. The method according to claim 55 autoimmune disease is selected from the group consisting of rheumatoid arthritis (RA), autoimmune pancreatitis (AIP), systemic lupus erythematosus (SLE), type I diabetes mellitus, multiple sclerosis (MS), antiphospholipid syndrome (APS), sclerosing cholangitis, systemic onset arthritis, irritable bowel disease (IBD), scleroderma, Sjogren's disease, vitiligo, polymyositis, pemphigus vulgaris, pemphigus foliaceus, inflammatory bowel disease including Crohn's disease and ulcerative colitis, autoimmune hepatitis, hypopituitarism, graft-versus-host disease (GvHD), autoimmune skin diseases, uveitis, pernicious anemia, and hypoparathyroidism. Autoimmune diseases may also include, without limitation, polyangiitis overlap syndrome, Kawasaki's disease, sarcoidosis, glomerulonephritis, and cryopathy.

58. The method according to claim 55 wherein the therapeutically effective amount of the compound is selected from a range consisting of 0.1 to 100 mg/kg of body weight of the patient, 0.1 to 50 mg/kg of body weight of the patient, 0.5 to 50 mg/kg of body weight of the patient, 1 to 20 mg/kg of body weight of the patient, 5 to 20 mg/kg of body weight of the patient, 10 to 20 mg/kg of body weight of the patient, 10 to 50 mg/kg of body weight of the patient, and 10 to 100 mg/kg of body weight of the patient.

59. The method according to claim 55 wherein the compound is administered to the patient continuously, multiple times daily, once daily, once every other day, weekly, bi-weekly, monthly, or bi-monthly.

60. The method according to claim 55 wherein the compound is administered orally, parenterally, by inhalation spray, topically, rectally, nasally, buccally, vaginally, or via an implanted reservoir.

61. The method according to claim 54 wherein the compound is administered subcutaneously, intravenously, intramuscularly, intra-articularly, intra-synovially, intrasternally, intrathecally, intrahepaticly, intralesionally, and by intracranial injection or infusion technique.

62. A method, comprising:
administering to a patient having an HPK1-mediated disorder a therapeutically effective amount of the pharmaceutical composition according to claim 53 or a pharmaceutically acceptable salt thereof.

63. The method according to claim 62 wherein the HPK1-mediated disorder is selected from the group consisting of cancer, autoimmune and/or inflammatory disease.

64. The method according to claim 63 wherein the cancer is selected from the group consisting of cancer of the breast, bladder, bone, brain, central and peripheral nervous system, colon, endocrine glands, esophagus, endometrium, germ cells, head and neck, kidney, liver, lung, larynx and hypopharynx, ovary, pancreas, prostate, rectum, renal, small intestine, soft tissue, testis, stomach, skin, ureter, vagina, and vulva.

65. The method according to claim 63 autoimmune disease is selected from the group consisting of rheumatoid arthritis (RA), autoimmune pancreatitis (AIP), systemic lupus erythematosus (SLE), type I diabetes mellitus, multiple sclerosis (MS), antiphospholipid syndrome (APS), sclerosing cholangitis, systemic onset arthritis, irritable bowel disease (IBD), scleroderma, Sjogren's disease, vitiligo, polymyositis, pemphigus vulgaris, pemphigus foliaceus, inflammatory bowel disease including Crohn's disease and ulcerative colitis, autoimmune hepatitis, hypopituitarism, graft-versus-host disease (GvHD), autoimmune skin diseases, uveitis, pernicious anemia, and hypoparathyroidism. Autoimmune diseases may also include, without limitation, polyangiitis overlap syndrome, Kawasaki's disease, sarcoidosis, glomerulonephritis, and cryopathy.

66. The method according to claim 63 wherein the therapeutically effective amount of the pharmaceutical composition is selected from a range consisting of 0.1 to 100 mg/kg of body weight of the patient, 0.1 to 50 mg/kg of body weight of the patient, 0.5 to 50 mg/kg of body weight of the patient, 1 to 20 mg/kg of body weight of the patient, 5 to 20 mg/kg of body weight of the patient, 10 to 20 mg/kg of body weight of the patient, 10 to 50 mg/kg of body weight of the patient, and 10 to 100 mg/kg of body weight of the patient.

67. The method according to claim 63 wherein the pharmaceutical composition is administered to the patient continuously, multiple times daily, once daily, once every other day, weekly, bi-weekly, monthly, or bi-monthly.

68. The method according to claim 63 wherein the compound is administered subcutaneously, intravenously, intramuscularly, intra-articularly, intra-synovially, intrasternally, intrathecally, intrahepaticly, intralesionally, and by intracranial injection or infusion technique.

69. (canceled)
